(19) United States
(12) Patent Application Publication
(10) Pub. No.: US 2024/0228504 A1
(43) Pub. Date: Jul. 11, 2024

(54) SUBSTITUTED TRICYCLIC AMIDES, ANALOGUES THEREOF, AND METHODS USING SAME

(71) Applicant: Arbutus Biopharma Corporation, Bumaby (CA)

(72) Inventors: Andrew G. Cole, Cranbury, NJ (US); Bruce D. Dorsey, Ambler, PA (US); Benjamin J. Dugan, Glen Mills, PA (US); Yi Fan, Doylestown, PA (US); Steven G. Kultgen, Warminster, PA (US); Eugen F. Mesaros, Wallingford, PA (US); Michael J. Sofia, Doylestown, PA (US)

(21) Appl. No.: 17/998,680

(22) PCT Filed: May 13, 2021
(86) PCT No.: PCT/IB21/00346

§ 371 Date: Nov. 14, 2022

Related U.S. Application Data
(60) Provisional application No. 63/024,559, filed on May 14, 2020.

(51) Int. Cl. C07D 491/052; A61K 31/4365; A61K 31/4709; A61K 31/4741; A61K 31/4743; A61K 31/4745; A61K 31/675; A61K 45/06; A61P 31/14; C07D 471/04; C07D 495/14; C07D 519/00; C07F 9/655

(52) U.S. Cl. CPC C07D 491/052; A61K 31/4365; A61K 31/4709; A61K 31/4741; A61K 31/4743; A61K 31/4745; A61K 31/675; A61K 45/06; A61P 31/14; C07D 471/04; C07D 495/14; C07D 519/00; C07F 9/6552

(57) ABSTRACT

The present invention includes substituted tricyclic amides of formula (I), or a salt, solvate, prodrug, stereoisomer, tautomer, or isotopically labelled derivative thereof, or any mixtures thereof, and compositions comprising the same. In certain embodiments, the compounds of the invention can be used to treat, ameliorate, and/or prevent hepatitis B virus (HBV) and/or hepatitis D virus (HDV) infections in a patient

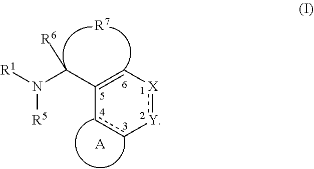

### CROSS-REFERENCE TO RELATED APPLICATIONS

This application claims priority under 35 U.S.C. § 119(e) to U.S. Provisional Patent Application No. 63/024,559, filed May 14, 2020, which is incorporated herein by reference in its entirety.

### BACKGROUND

Hepatitis B is one of the world's most prevalent diseases, being listed by National Institute of Allergy and Infectious Diseases (NIAID) as a High Priority Area of Interest. Although most individuals resolve the infection following acute symptoms, approximately 30% of cases become chronic. 350-400 million people worldwide are estimated to have chronic hepatitis B, leading to 0.5-1 million deaths per year, due largely to the development of hepatocellular carcinoma, cirrhosis and/or other complications.

A limited number of drugs are currently approved for the management of chronic hepatitis B, including two formulations of alpha-interferon (standard and pegylated) and five nucleoside/nucleotide analogues (lamivudine, adefovir, entecavir, telbivudine, and tenofovir) that inhibit hepatitis B virus (HBV) DNA polymerase. At present, the first-line treatment choices are entecavir, tenofovir and/or peg-interferon alfa-2a. However, peg-interferon alfa-2a achieves desirable serological milestones in only one third of treated patients, and is frequently associated with severe side effects. Entecavir and tenofovir are potent HBV inhibitors, but require long-term or possibly lifetime administration to continuously suppress HBV replication, and may eventually fail due to emergence of drug-resistant viruses. There is thus a pressing need for the introduction of novel, safe, and effective therapies for chronic hepatitis B.

HBV is a noncytopathic, liver tropic DNA virus belonging to Hepadnaviridae family. Pregenomic (pg) RNA is the template for reverse transcriptional replication of HBV DNA. The encapsidation of pg RNA, together with viral DNA polymerase, into a nucleocapsid is essential for the subsequent viral DNA synthesis. Inhibition of pg RNA encapsidation may block HBV replication and provide a new therapeutic approach to HBV treatment. A capsid inhibitor acts by inhibiting the expression and/or function of a capsid protein either directly or indirectly: for example, it may inhibit capsid assembly, induce formation of non-capsid polymers, promote excess capsid assembly or misdirected capsid assembly, affect capsid stabilization, and/or inhibit RNA encapsidation. A capsid inhibitor may also act by inhibiting capsid function in one or more downstream events within the replication process, such as, but not limited to, viral DNA synthesis, transport of relaxed circular DNA (rcDNA) into the nucleus, covalently closed circular DNA (cccDNA) formation, virus maturation, budding and/or release.

Clinically, inhibition of pg RNA encapsidation, or more generally inhibition of nucleocapsid assembly, may offer certain therapeutic advantages. In one aspect, inhibition of pg RNA encapsidation may complement the current medications by providing an option for a subpopulation of patients that do not tolerate or benefit from the current medications. In another aspect, based on their distinct antiviral mechanism, inhibition of pg RNA encapsidation may be effective against HBV variants resistant to the currently available DNA polymerase inhibitors. In yet another aspect, combination therapy of the pg RNA encapsidation inhibitors with DNA polymerase inhibitors may synergistically suppress HBV replication and prevent drug resistance emergence, thus offering a more effective treatment for chronic hepatitis B infection.

Hepatitis D virus (HDV) is a small circular enveloped RNA virus that can propagate only in the presence of HBV. In particular, HDV requires the HBV surface antigen protein to propagate itself. Infection with both HBV and HDV results in more severe complications compared to infection with HBV alone. These complications include a greater likelihood of experiencing liver failure in acute infections and a rapid progression to liver cirrhosis, with an increased chance of developing liver cancer in chronic infections. In combination with hepatitis B, hepatitis D has the highest mortality rate of all the hepatitis infections. The routes of transmission of HDV are similar to those for HBV. Infection is largely restricted to persons at high risk of HBV infection, particularly injecting drug users and persons receiving clotting factor concentrates.

Currently, there is no effective antiviral therapy available for the treatment of acute or chronic type D hepatitis. Interferon-alfa given weekly for 12 to 18 months is the only licensed treatment for hepatitis D. Response to this therapy is limited, as only about one-quarter of patients is serum HDV RNA undetectable 6 months post therapy.

Clinically, inhibition of pg RNA encapsidation, or more generally inhibition of nucleocapsid assembly, may offer certain therapeutic advantages for treatment of hepatitis B and/or hepatitis D. In one aspect, inhibition of pg RNA encapsidation may complement the current medications by providing an option for a subpopulation of patients that do not tolerate or benefit from the current medications. In another aspect, based on their distinct antiviral mechanism, inhibition of pg RNA encapsidation may be effective against HBV and/or HDV variants resistant to the currently available DNA polymerase inhibitors. In yet another aspect, combination therapy of the pg RNA encapsidation inhibitors with DNA polymerase inhibitors may synergistically suppress HBV and/or HDV replication and prevent drug resistance emergence, thus offering a more effective treatment for chronic hepatitis B and/or hepatitis D infection.

There is thus a need in the art for the identification of novel compounds that can be used to treat and/or prevent HBV and/or HDV infection in a subject. In certain embodiments, the novel compounds inhibit HBV and/or HDV nucleocapsid assembly. In other embodiments, the novel compounds can be used in patients that are HBV and/or HBV-HDV infected, patients who are at risk of becoming HBV and/or HBV-HDV infected, and/or patients that are infected with drug-resistant HBV and/or HDV. The present disclosure addresses this need.

### BRIEF SUMMARY

The present disclosure provides certain compounds of formula (I), or a salt, solvate, prodrug, stereoisomer, tautomer, or isotopically labeled derivative thereof, or any mixture thereof, wherein the substituents in (I) are defined elsewhere herein:

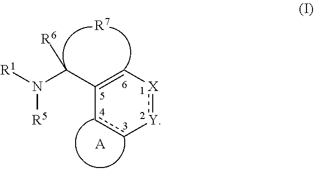

The present disclosure further provides pharmaceutical compositions comprising at least one compound of the present disclosure. In certain embodiments, the pharmaceutical compositions further comprise at least one pharmaceutically acceptable carrier. In other embodiments, the pharmaceutical compositions further comprise at least one additional agent that treats or prevents hepatitis virus infection. In yet other embodiments, the hepatitis virus is hepatitis B virus (HBV). In yet other embodiments, the hepatitis virus is hepatitis D virus (HDV).

The present disclosure further provides a method of treating, ameliorating, and/or preventing hepatitis virus infection in a subject. In certain embodiments, the method comprises administering to the subject a therapeutically effective amount of a compound of the invention, or a salt, solvate, prodrug, stereoisomer, tautomer, or any mixtures thereof. In other embodiments, the subject is infected with HBV. In yet other embodiments, the subject is infected with HDV. In yet other embodiments, the subject is infected with HBV and HDV. In yet other embodiments, the subject is further administered at least one additional agent useful for treating, ameliorating, and/or preventing the hepatitis virus infection. In yet other embodiments, the subject is in need of the treatment, amelioration, and/or prevention.

### BRIEF DESCRIPTION OF THE DRAWINGS

The following detailed description of illustrative embodiments of the disclosure will be better understood when read in conjunction with the appended drawings. For the purpose of illustrating the disclosure, exemplary embodiments are shown in the drawing(s). It should be understood, however, that the disclosure is not limited to the precise arrangements and instrumentalities of the embodiments shown in the drawings.

FIG. 1 provides the ORTEP representation of (S)-8,9-Difluoro-1-(((R)-1-(4-methoxyphenyl)ethyl)amino)-1,5-dihydro-2H-pyrano[3,4-c]isoquinolin-6(4H)-one with 50% probability thermal ellipsoids displayed, defining the absolute configuration.

### DETAILED DESCRIPTION

The disclosure relates, in certain aspects, to the discovery of certain substituted ureas and amides that are useful to treat and/or prevent hepatitis B virus (HBV) and/or hepatitis D virus (HDV) infection and related conditions in a subject. In certain embodiments, the compounds of the disclosure are viral capsid inhibitors.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. A specific embodiment is (C₁-C₆)alkyl, such as, but not limited to, ethyl, methyl, isopropyl, isobutyl, n-pentyl, n-hexyl, and cyclopropylmethyl.

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH.

As used herein, the term “aromatic” refers to a carbocycle or heterocycle with one or more polyunsaturated rings and having aromatic character, i.e., having (4n+2) delocalized π (pi) electrons, where ‘n’ is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” refers to a functional group wherein a one-to-six carbon alkylene chain is attached to an aryl group, e.g., —CH₂CH₂-phenyl or —CH₂-phenyl (or benzyl). Specific examples are aryl-CH₂— and aryl-CH(CH₃)—. The term “substituted aryl-(C₁-C₆)alkyl” refers to an aryl-(C₁-C₆)alkyl functional group in which the aryl group is substituted. A specific example is substituted aryl(CH₂)—. Similarly, the term “heteroaryl-(C₁-C₆)alkyl” refers to a functional group wherein a one-to-three carbon alkylene chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)—.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “halide” refers to a halogen atom bearing a negative charge. The halide anions are fluoride (F⁻), chloride (Cl⁻), bromide (Br⁻), and iodide (I⁻).

As used herein, the term “halo” or “halogen” alone or as part of another substituent refers to, unless otherwise stated, a fluorine, chlorine, bromine, or iodine atom.

As used herein, the term “heteroalkenyl” by itself or in combination with another term refers to, unless otherwise stated, a stable straight or branched chain monounsaturated or diunsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NH—OCH₃, or —CH₂CH₂SSCH₃.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl, and 1,3,4-oxadiazolyl.

Examples of polycyclic heterocycles include indolyl (such as, but not limited to, 3-, 4-5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

By the term “specifically bind” or “specifically binds” as used herein is meant that a first molecule preferentially binds to a second molecule (e.g., a particular receptor or enzyme), but does not necessarily bind only to that second molecule.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “substituted” refers to that an atom or group of atoms has replaced hydrogen as the substituent attached to another group.

As used herein, the term “substituted alkyl,” “substituted cycloalkyl,” “substituted alkenyl,” or “substituted alkynyl” refers to alkyl, cycloalkyl, alkenyl, or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C≡N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH. Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet other embodiments, the substituents are independently selected from the group consisting of C₁-C₆ alkyl, —OH, C₁-C₆ alkoxy, halo, amino, acetamido and nitro. As used herein, where a substituent is an alkyl or alkoxy group, the carbon chain may be branched, straight or cyclic.

Unless otherwise noted, when two substituents are taken together to form a ring having a specified number of ring atoms (e.g., Rⁱ and Rⁱⁱ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The terms “treat,” “treating” and “treatment,” as used herein, means reducing the frequency or severity with which symptoms of a disease or condition are experienced by a subject by virtue of administering an agent or compound to the subject.

Certain abbreviations used herein follow: ACN, acetonitrile; cccDNA, covalently closed circular DNA; DAD, diode array detector; DCE, 1,2-dichloroethane; DCM, dichloromethane; DIEA or DIPEA, diisopropylethylamine; DMF, N,N-dimethylformamide; DMSO, dimethylsulfoxide; d.r., diastereomeric ratio; EtOAc, ethyl acetate; HATU, hexafluorophosphate azabenzotriazole tetramethyl uronium; HBsAg, HBV surface antigen; HBV, hepatitis B virus; HDV, hepatitis D virus; HPLC, high pressure liquid chromatography; IPA, isopropanol (2-propanol); LCMS, liquid chromatography mass spectrometry; LG, leaving group; NARTI or NRTI, reverse-transcriptase inhibitor; NMM, N-methylmorpholine; NMR, Nuclear Magnetic Resonance; NtARTI or NtRTI, nucleotide analog reverse-transcriptase inhibitor; pg RNA, pregenomic RNA; rcDNA, relaxed circular DNA; RT, retention time; sAg, surface antigen; SFC, supercritical fluid chromatography; STAB, sodium triacetoxyborohydride; TFA, trifluoroacetic acid; TBDMS, tert-butyldimethylsilyl; THF, tetrahydrofuran; TLC, thin layer chromatography; TMSOTf, trimethylsilyl trifluoromethylsulfonate.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. This applies regardless of the breadth of the range.

### Compounds

The disclosure includes a compound of formula (I), or a salt, solvate, prodrug, isotopically labelled derivative, stereoisomer (such as, in a non-limiting example, an enantiomer or diastereoisomer, and/or any mixtures thereof, such as, in a non-limiting example, mixtures in any proportions of enantiomers and/or diastereoisomers thereof), tautomer and any mixtures thereof, and/or geometric isomer and any mixtures thereof:

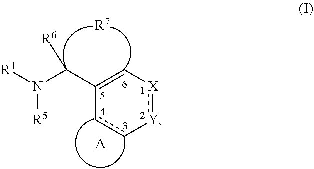

wherein:
   X, Y, and the bond between X and Y are such that:
      X is NR⁸, Y is C(═O), and the bond between X and Y is a single bond, or
      X is N, Y is CR¹¹, and the bond between X and Y is a double bond;
   ring A is selected from the group consisting of:

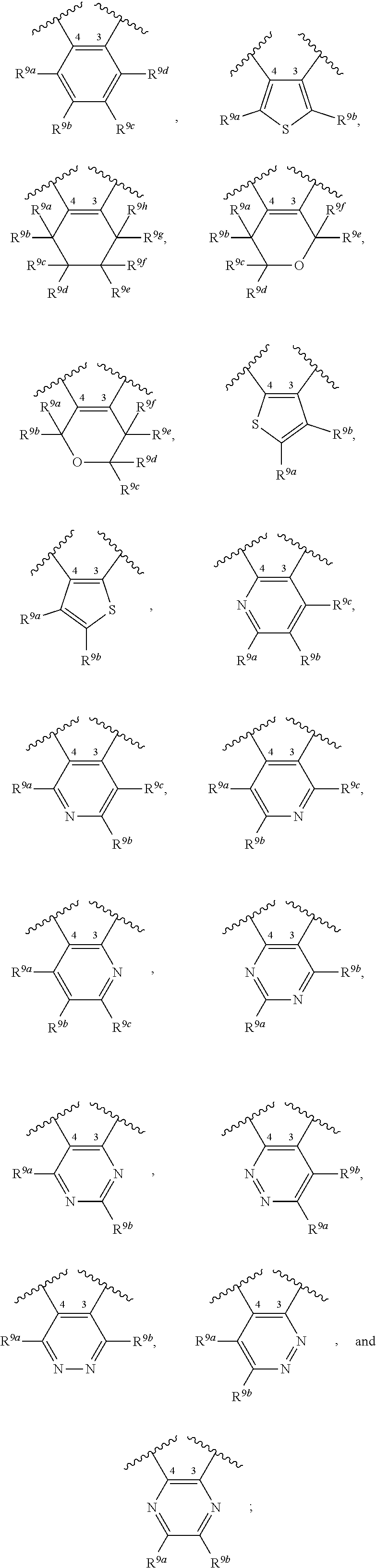

R¹ is selected from the group consisting of R²C(═O)—, R²S(═O)₂—, and naturally occurring aminoacyl;
R² is selected from the group consisting of

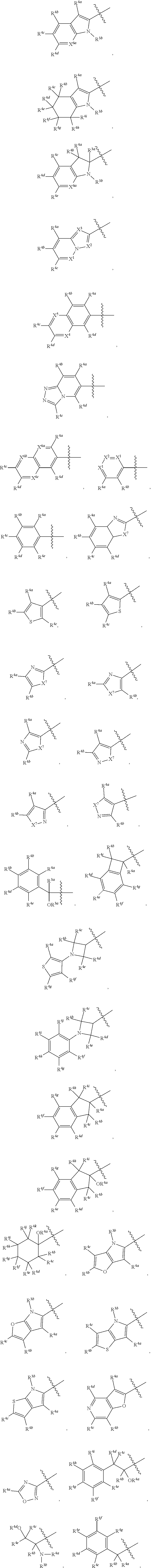

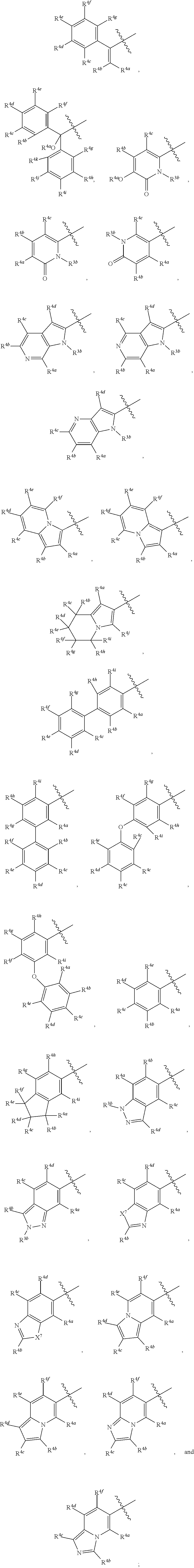

one of the following applies:
   (i) X¹ is N, X² is C(R^{4f}), and X³ is C(R^{4g});
   (ii) X² is N, X¹ is C(R^{4f}), and X³ is C(R^{4g});
   (iii) X³ is N, X¹ is C(R^{4f}), and X² is C(R^{4g});
   (v) X¹ is C(R^{4f}), X² is C(R^{4g}), and X³ is C(R^{4h});
one of the following applies:
   (i) X⁴ is N and X⁵ is C(R^{4e}); or
   (ii) X⁵ is N and X⁴ is C(R^{4e});
each occurrence of X^{6a} is independently N or C(R^{4f});
each occurrence of X^{6b} is independently N or C(R^{4g});
each occurrence of X^{6c} is independently N or C(R^{4h});
each occurrence of X⁷ is independently S, O, or NR^{3a};
each occurrence of R^{3a} is independently selected from the group consisting of H, optionally substituted C₁-C₆ alkyl, and optionally substituted C₃-C₈ cycloalkyl;
each occurrence of R^{3b} is independently selected from the group consisting of H, optionally substituted C₁-C₆ alkyl, and optionally substituted C₃-C₈ cycloalkyl;
each occurrence of R^{4a}, R^{4b}, R^{4c}, R^{4d}, R^{4e}, R^{4f}, R^{4g}, R^{4h}, R⁴ⁱ, R^{4j} and R^{4k} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, optionally substituted phenyl, optionally substituted heterocyclyl, optionally substituted heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R⁵ is independently selected from the group consisting of H, C₁-C₆ alkyl, and C₃-C₈ cycloalkyl, wherein the alkyl or cycloalkyl is optionally substituted with at least one selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, optionally substituted phenyl, optionally substituted heteroaryl, optionally substituted heterocyclyl, —C(═O)OR¹⁰, —OC(═O)R¹⁰, —SR¹⁰, —S(═O)R¹⁰, —S(═O)₂R¹⁰, —S(═O)₂NR¹⁰R¹⁰, —N(R¹⁰)S(═O)₂R¹⁰, —N(R¹⁰)C(═O)R¹⁰, —C(═O)NR¹⁰R¹⁰, and —NR¹⁰R¹⁰;
each occurrence of R⁶ is independently selected from the group consisting of H and optionally substituted C₁-C₆ alkyl;
R⁷ is —(CH₂)ₚ-Q-(CH₂)_{q}—, wherein p and q are independently 0, 1, or 2, and Q is a bond (absent), —O—, —S—, —S(O)—, —S(O)₂—, —NR¹², —CH(OH)—, —C(═O)—, —C(═O)O—, or —OC(═O)—,
   wherein 2≤(p+q)≤4 if Q is a bond,
   wherein 1≤(p+q)≤3 if Q is —O—, S—, —S(O)—, —S(O)₂—, —NR¹², —CH(OH)—, or —C(═O)—,
   wherein 0≤(p+q)≤2 if Q is —C(═O)O— or —OC(═O)—, and
   wherein each CH₂ in R⁷ is optionally substituted with at least one substituent selected from the group consisting of methyl, OR¹³, or halogen;
each occurrence of R⁸ is independently selected from the group consisting of H, optionally substituted C₁-C₆ alkyl, and optionally substituted C₃-C₈ cycloalkyl;
each occurrence of R^{9a}, R^{9b}, R^{9c}, R^{9d}, R^{9e}, R^{9f}, R^{9g}, and R^{9h} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, optionally substituted phenyl, optionally substituted heterocyclyl, optionally substituted heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R¹⁰ is independently selected from the group consisting of H, optionally substituted C₁-C₆ alkyl, optionally substituted C₃-C₈ cycloalkyl, optionally substituted phenyl, and optionally substituted hetereoaryl;
each occurrence of R¹¹ is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, optionally substituted phenyl, optionally substituted heterocyclyl, optionally substituted heteroaryl, —S(optionally substituted C₁-C₆ alkyl), —SO(optionally substituted C₁-C₆ alkyl), —SO₂(optionally substituted C₁-C₆ alkyl), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R¹² is independently selected from the group consisting of H, optionally substituted C₁-C₆ alkyl, optionally substituted C₁-C₆ hydroxyalkyl, optionally substituted C₃-C₈ cycloalkyl, optionally substituted phenyl, optionally substituted heteroaryl, and optionally substituted C₁-C₆ acyl;
each occurrence of R¹³ is independently selected from the group consisting of H, optionally substituted C₁-C₆ alkyl, and —C(═O)C₁-C₆ alkyl.

In certain embodiments, the compound of formula (I) is a compound of formula (Ia-1):

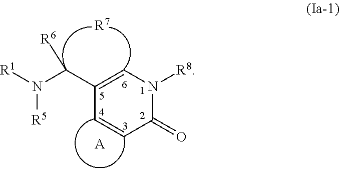

In certain embodiments, the compound of formula (I) is a compound of formula (Ia-2):

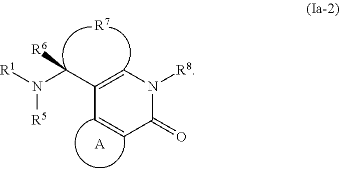

In certain embodiments, the compound of formula (I) is a compound of formula (Ia-3):

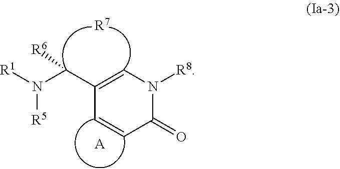

In certain embodiments, the compound of formula (I) is a compound of formula (Ia-4):

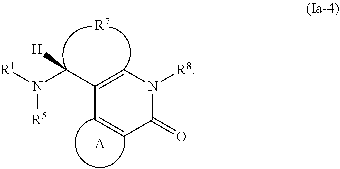

In certain embodiments, the compound of formula (I) is a compound of formula (Ia-5):

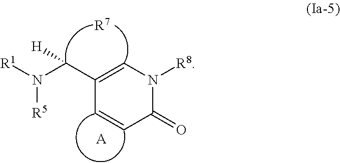

In certain embodiments, the compound of formula (I) is a compound of formula (Ia-6):

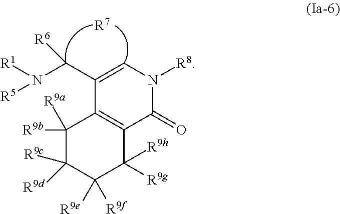

In certain embodiments, the compound of formula (I) is a compound of formula (Ia-7):

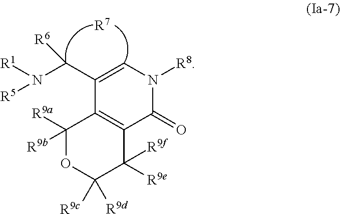

In certain embodiments, the compound of formula (I) is a compound of formula (Ia-8):

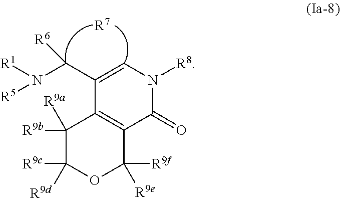

In certain embodiments, the compound of formula (I) is a compound of formula (Ia-9):

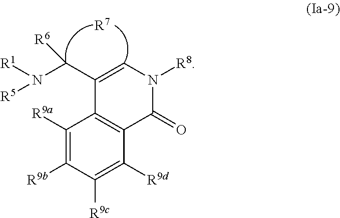

In certain embodiments, the compound of formula (I) is a compound of formula (Ia-10):

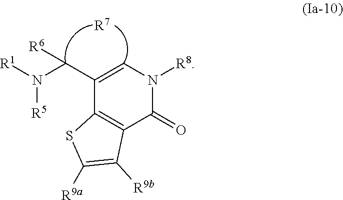

In certain embodiments, the compound of formula (I) is a compound of formula (Ia-11):

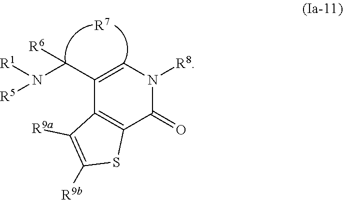

In certain embodiments, the compound of formula (I) is a compound of formula (Ia-12):

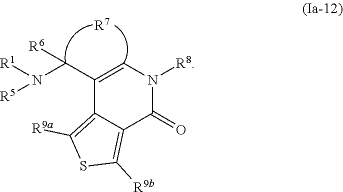

In certain embodiments, the compound of formula (I) is a compound of formula (Ib-1):

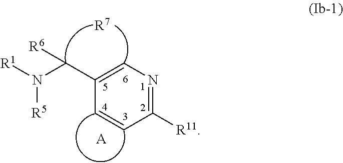

In certain embodiments, the compound of formula (I) is a compound of formula (Ib-2):

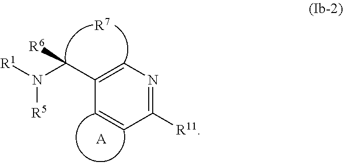

In certain embodiments, the compound of formula (I) is a compound of formula (Ib-3).

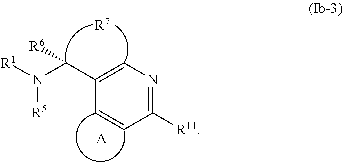

In certain embodiments, the compound of formula (I) is a compound of formula (Ib-4):

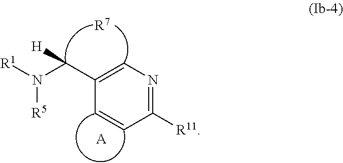

In certain embodiments, the compound of formula (I) is a compound of formula (Ib-5):

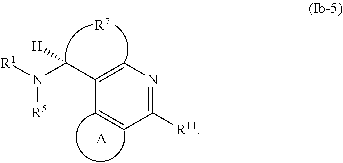

In certain embodiments, the compound of formula (I) is a compound of formula (Ib-6):

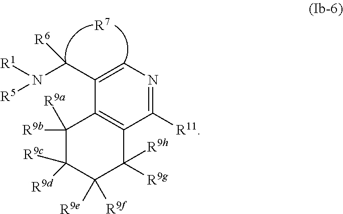

In certain embodiments, the compound of formula (I) is a compound of formula (Ib-7):

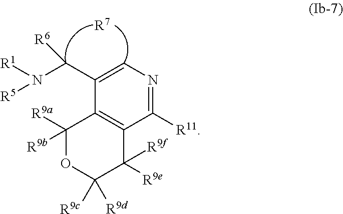

In certain embodiments, the compound of formula (I) is a compound of formula (Ib-8):

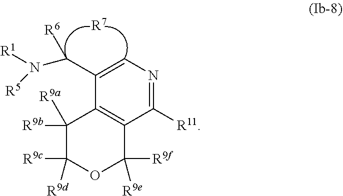

In certain embodiments, the compound of formula (I) is a compound of formula (Ib-9):

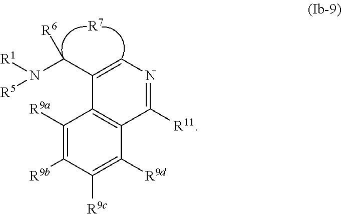

In certain embodiments, the compound of formula (I) is a compound of formula (Ib-10):

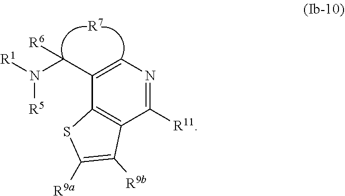

In certain embodiments, the compound of formula (I) is a compound of formula (Ib-11):

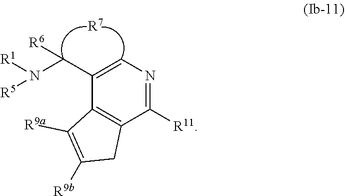

In certain embodiments, the compound of formula (I) is a compound of formula (Ib-12):

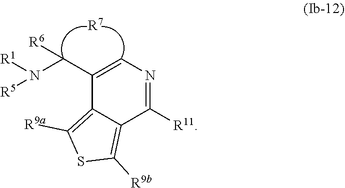

In certain embodiments, each occurrence of alkyl, alkenyl, alkynyl, or cycloalkyl is independently optionally substituted with at least one substituent selected from the group consisting of C₁-C₆ alkyl, C₃-C₈ cycloalkyl, halo, cyano (—CN), —OR^{a}, optionally substituted phenyl (thus yielding, in non-limiting examples, optionally substituted phenyl-(C₁-C₃ alkyl), such as, but not limited to, benzyl or substituted benzyl), optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

In certain embodiments, each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —C(═O)OR^{b}, —OC(═O)R^{b}, —SR^{b}, —S(═O)R^{b}, —S(═O)₂R^{b}, —N(R^{b})S(═O)₂R^{b}, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

In certain embodiments, each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —OR^{b}, —C(═O)N(R^{b})(R^{b}), —C(═O)OR^{b}, —OC(═O)R^{b}, —SR^{b}, —S(═O)R^{b}, —S(═O)₂R^{b}, and —N(R^{b})S(═O)₂R^{b}, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

In certain embodiments, the alkyl, alkenyl, alkynyl, cycloalkyl, heteroaryl, heterocyclyl, aryl, or benzyl group is optionally independently substituted with at least one group selected from the group consisting of C₁-C₆ alkyl; C₁-C₆ alkoxy; C₁-C₆ haloalkyl; C₁-C₆ haloalkoxy; —NH₂, —NH(C₁-C₆ alkyl), —N(C₁-C₆ alkyl)(C₁-C₆ alkyl), halogen, —OH; —CN; phenoxy, —NHC(═O)H, —NHC(═O)C₁-C₆ alkyl, —C(═O)NH₂, —C(═O)NHC₁-C₆ alkyl, —C(═O)N(C₁-C₆ alkyl)(C₁-C₆ alkyl), tetrahydropyranyl, morpholinyl, —C(═O)CH₃, —C(═O)CH₂OH, —C(═O)NHCH₃, —C(═O)CH₂OMe, or an N-oxide thereof.

In certain embodiments, each occurrence of the heteroaryl is independently selected from the group consisting of quinolinyl, imidazo[1,2-a]pyridyl, pyridyl, pyrimidyl, pyrazinyl, imidazolyl, thiazolyl, pyrazolyl, isoxazolyl, oxadiazolyl (including 1,2,3-, 1,2,4-, 1,2,5-, and 1,3,4-oxadiazole), and triazolyl (such as 1,2,3-triazolyl and 1,2,4-triazolyl).

In certain embodiments, each occurrence of the heterocyclyl group is independently selected from the group consisting of tetrahydrofuranyl, tetrahydropyranyl, piperidinyl, piperazinyl, pyrrolidinyl, morpholinyl, thiomorpholinyl, 1-oxido-thiomorpholinyl, 1,1-dioxido-thiomorpholinyl, oxazolidinyl, azetidinyl, and the corresponding oxo analogues (where a methylene ring group is replaced with a carbonyl) thereof.

In certain embodiments, the aminoacyl comprises a naturally occurring aminoacyl, or an enantiomer or diastereoisomer thereof, such as but not limited to glycyl, alanyl, valinyl, leucyl, isoleucyl, prolyl, seryl, threonyl, cysteinyl, cystinyl, methionyl, phenylalanyl, tryptophanyl, tyrosyl, aspartyl, glutamyl, asparagyl, glutamyl, lysyl, arginyl, and/or histidyl.

In certain embodiments, R² is

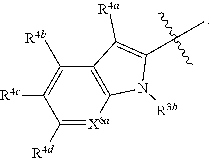

In certain embodiments, R² is

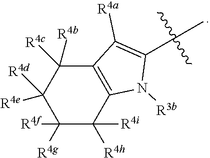

In certain embodiments, R² is

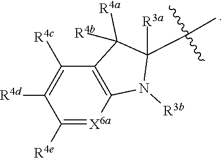

In certain embodiments, R² is

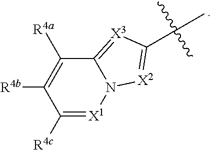

In certain embodiments, R² is

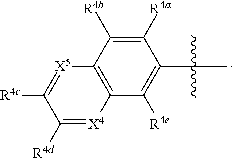

In certain embodiments, R² is

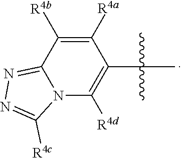

In certain embodiments, R² is

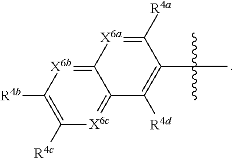

In certain embodiments, R² is

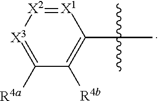

In certain embodiments, R² is

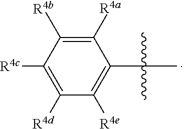

In certain embodiments, R² is

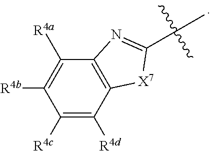

In certain embodiments, R² is

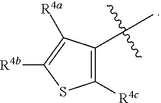

In certain embodiments, R² is

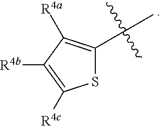

In certain embodiments, R² is

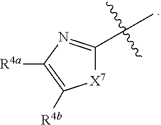

In certain embodiments, R² is

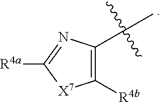

In certain embodiments, R² is

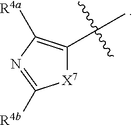

In certain embodiments, R² is

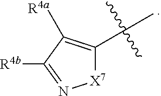

In certain embodiments, R² is

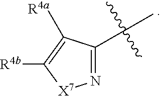

In certain embodiments, R² is

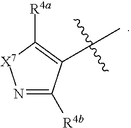

In certain embodiments, R² is

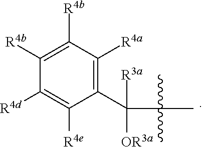

In certain embodiments, R² is

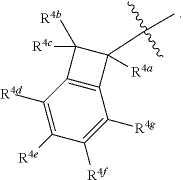

In certain embodiments, R² is

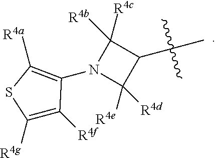

In certain embodiments, R² is

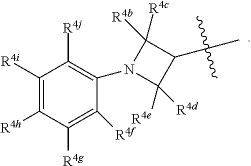

In certain embodiments, R² is

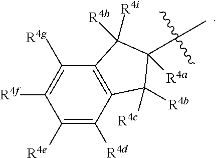

In certain embodiments, R² is

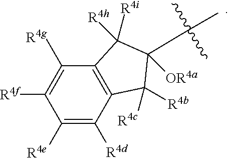

In certain embodiments, R² is

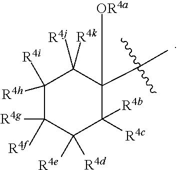

In certain embodiments, R² is

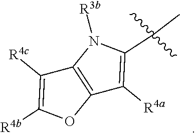

In certain embodiments, R² is

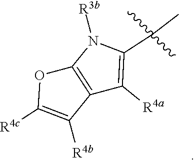

In certain embodiments, R² is

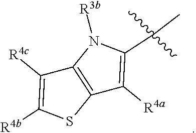

In certain embodiments, R² is

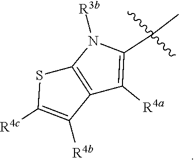

In certain embodiments, R² is

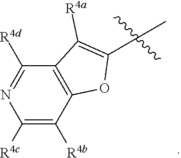

In certain embodiments, R² is

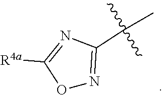

In certain embodiments, R² is

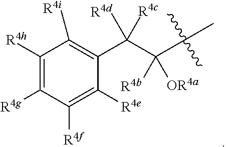

In certain embodiments, R² is

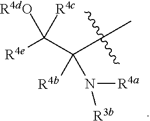

In certain embodiments, R² is

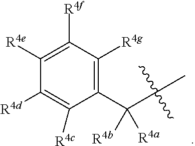

In certain embodiments, R² is

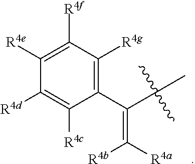

In certain embodiments, R² is

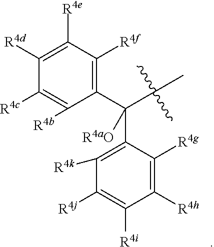

In certain embodiments, R² is

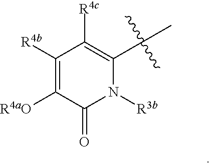

In certain embodiments, R² is

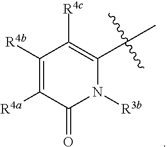

In certain embodiments, R² is

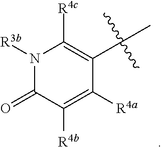

In certain embodiments, R² is

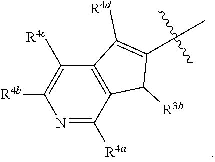

In certain embodiments, R² is

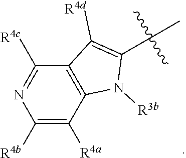

In certain embodiments, R² is

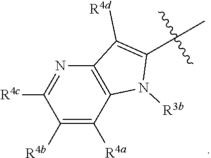

In certain embodiments, R² is

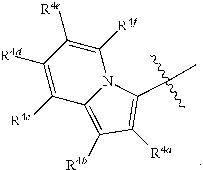

In certain embodiments, R² is

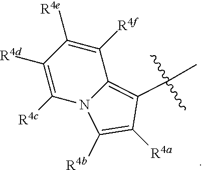

In certain embodiments, R² is

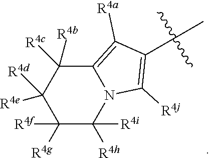

In certain embodiments, R² is

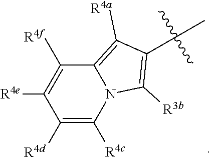

In certain embodiments, R² is

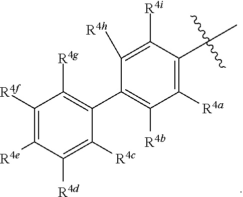

In certain embodiments, R² is

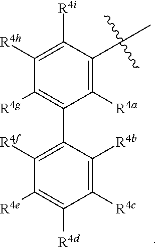

In certain embodiments, R² is

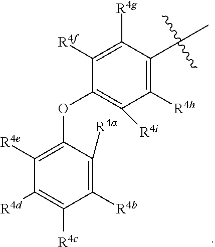

In certain embodiments, R² is

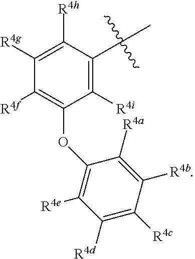

In certain embodiments, R² is

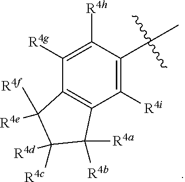

In certain embodiments, R² is

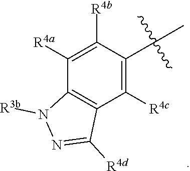

In certain embodiments, R² is

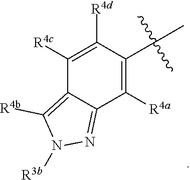

In certain embodiments, R² is

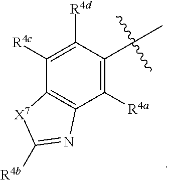

In certain embodiments, R² is

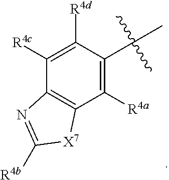

In certain embodiments, R² is

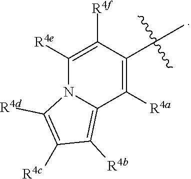

In certain embodiments, R² is

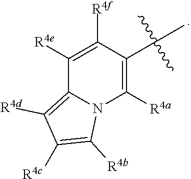

In certain embodiments, R² is

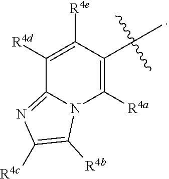

In certain embodiments, R² is

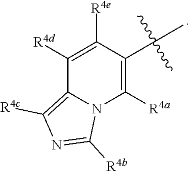

In certain embodiments, R² is

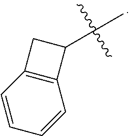 [SMILES: *C1Cc2ccccc21]

In certain embodiments, R² is

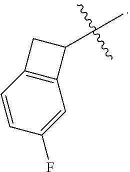 [SMILES: *C1Cc2ccc(F)cc21]

In certain embodiments, R² is

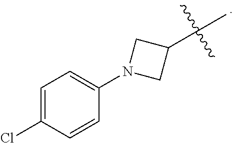 [SMILES: *C1CN(c2ccc(Cl)cc2)C1]

In certain embodiments, R² is

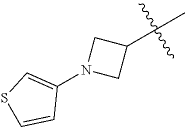 [SMILES: *C1CN(c2ccsc2)C1]

In certain embodiments, R² is

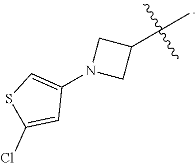 [SMILES: *C1CN(c2csc(Cl)c2)C1]

In certain embodiments, R² is

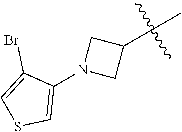 [SMILES: *C1CN(c2cscc2Br)C1]

In certain embodiments, R² is

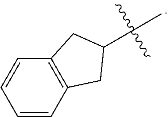 [SMILES: *C1Cc2ccccc2C1]

In certain embodiments, R² is

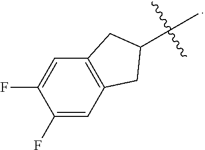 [SMILES: *C1Cc2cc(F)c(F)cc2C1]

In certain embodiments, R² is

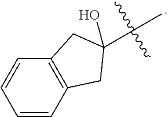 [SMILES: *C1(O)Cc2ccccc2C1]

In certain embodiments, R² is

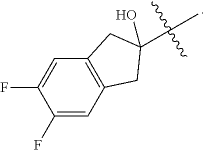 [SMILES: *C1(O)Cc2cc(F)c(F)cc2C1]

In certain embodiments, R² is

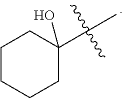 [SMILES: *C1(O)CCCCC1]

In certain embodiments, R² is

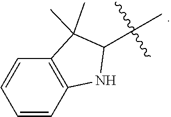 [SMILES: *C1Nc2ccccc2C1(C)C]

In certain embodiments, R² is

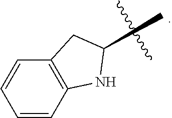

In certain embodiments, R² is

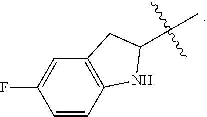 [SMILES: *C1Cc2cc(F)ccc2N1]

In certain embodiments, R² is

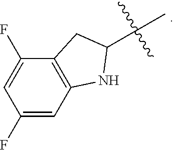 [SMILES: *C1Cc2c(F)cc(F)cc2N1]

In certain embodiments, R² is

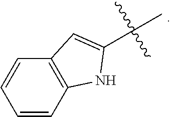 [SMILES: *c1cc2ccccc2[nH]1]

In certain embodiments, R² is

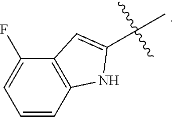 [SMILES: *c1cc2c(F)cccc2[nH]1]

In certain embodiments, R² is

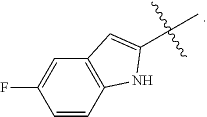 [SMILES: *c1cc2cc(F)ccc2[nH]1]

In certain embodiments, R² is

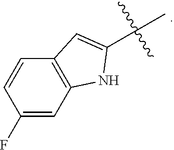 [SMILES: *c1cc2ccc(F)cc2[nH]1]

In certain embodiments, R² is

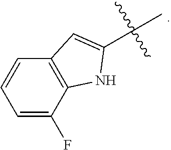 [SMILES: *c1cc2cccc(F)c2[nH]1]

In certain embodiments, R² is

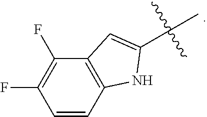 [SMILES: *c1cc2c(F)c(F)ccc2[nH]1]

In certain embodiments, R² is

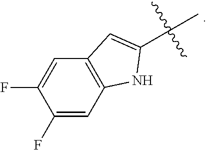 [SMILES: *c1cc2cc(F)c(F)cc2[nH]1]

In certain embodiments, R² is

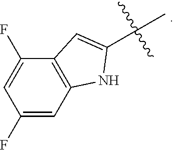 [SMILES: *c1cc2c(F)cc(F)cc2[nH]1]

In certain embodiments, R² is

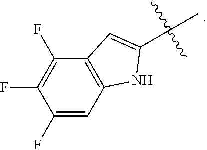 [SMILES: *c1cc2c(F)c(F)c(F)cc2[nH]1]

In certain embodiments, R² is

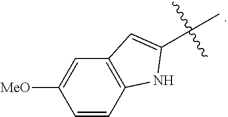 [SMILES: *c1cc2cc(OC)ccc2[nH]1]

In certain embodiments, R² is

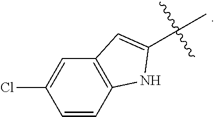 [SMILES: *c1cc2cc(Cl)ccc2[nH]1]

In certain embodiments, R² is

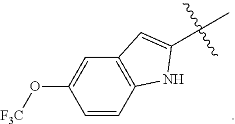 [SMILES: *c1cc2cc(OC(F)(F)F)ccc2[nH]1]

In certain embodiments, R² is

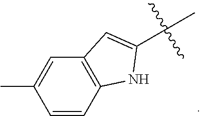 [SMILES: *c1cc2cc(C)ccc2[nH]1]

In certain embodiments, R² is

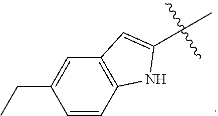 [SMILES: *c1cc2cc(CC)ccc2[nH]1]

In certain embodiments, R² is

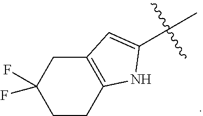 [SMILES: *c1cc2c([nH]1)CCC(F)(F)C2]

In certain embodiments, R² is

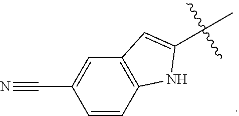 [SMILES: *c1cc2cc(C#N)ccc2[nH]1]

In certain embodiments, R² is

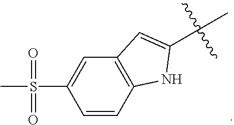 [SMILES: *c1cc2cc(S(C)(=O)=O)ccc2[nH]1]

In certain embodiments, R² is

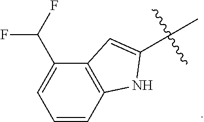 [SMILES: *c1cc2c(C(F)F)cccc2[nH]1]

In certain embodiments, R² is

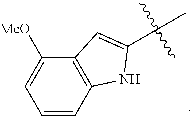 [SMILES: *c1cc2c(OC)cccc2[nH]1]

In certain embodiments, R² is

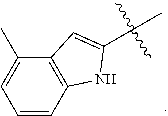 [SMILES: *c1cc2c(C)cccc2[nH]1]

In certain embodiments, R² is

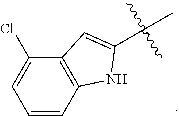 [SMILES: *c1cc2c(Cl)cccc2[nH]1]

In certain embodiments, R² is

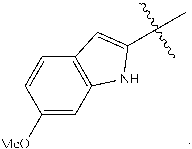 [SMILES: *c1cc2ccc(OC)cc2[nH]1]

In certain embodiments, R² is

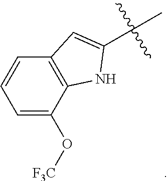 [SMILES: *c1cc2cccc(OC(F)(F)F)c2[nH]1]

In certain embodiments, R² is

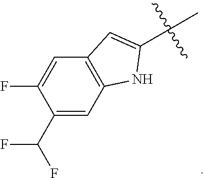 [SMILES: *c1cc2cc(F)c(C(F)F)cc2[nH]1]

In certain embodiments, R² is

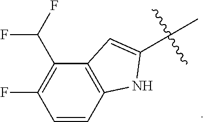 [SMILES: *c1cc2c(C(F)F)c(F)ccc2[nH]1]

In certain embodiments, R² is

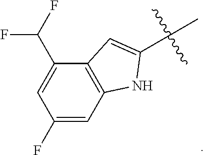 [SMILES: *c1cc2c(C(F)F)cc(F)cc2[nH]1]

In certain embodiments, R² is

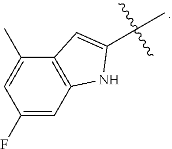 [SMILES: *c1cc2c(C)cc(F)cc2[nH]1]

In certain embodiments, R² is

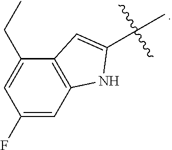 [SMILES: *c1cc2c(CC)cc(F)cc2[nH]1]

In certain embodiments, R² is

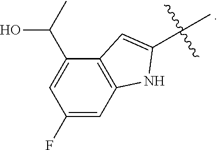 [SMILES: *c1cc2c(C(C)O)cc(F)cc2[nH]1]

In certain embodiments, R² is

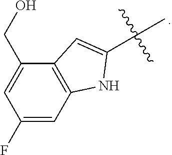 [SMILES: *c1cc2c(CO)cc(F)cc2[nH]1]

In certain embodiments, R² is

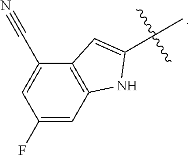 [SMILES: *c1cc2c(C#N)cc(F)cc2[nH]1]

In certain embodiments, R² is

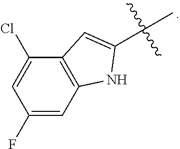 [SMILES: *c1cc2c(Cl)cc(F)cc2[nH]1]

In certain embodiments, R² is

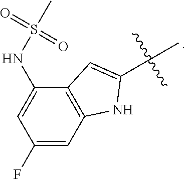 [SMILES: *c1cc2c(NS(C)(=O)=O)cc(F)cc2[nH]1]

In certain embodiments, R² is

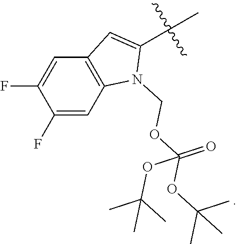

In certain embodiments, R² is

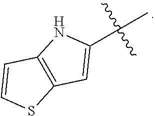 [SMILES: *c1cc2sccc2[nH]1]

In certain embodiments, R² is

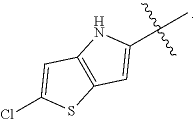 [SMILES: *c1cc2sc(Cl)cc2[nH]1]

In certain embodiments, R² is

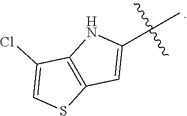 [SMILES: *c1cc2scc(Cl)c2[nH]1]

In certain embodiments, R² is

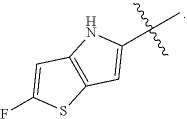 [SMILES: *c1cc2sc(F)cc2[nH]1]

In certain embodiments, R² is

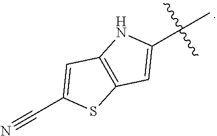 [SMILES: *c1cc2sc(C#N)cc2[nH]1]

In certain embodiments, R² is

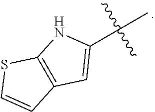 [SMILES: *c1cc2ccsc2[nH]1]

In certain embodiments, R² is

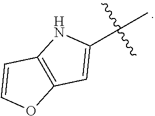 [SMILES: *c1cc2occc2[nH]1]

In certain embodiments, R² is

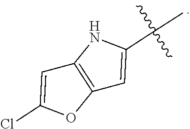 [SMILES: *c1cc2oc(Cl)cc2[nH]1]

In certain embodiments, R² is

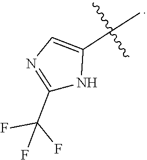 [SMILES: *c1cnc(C(F)(F)F)[nH]1]

In certain embodiments, R² is

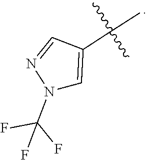 [SMILES: *c1cnn(C(F)(F)F)c1]

In certain embodiments, R² is

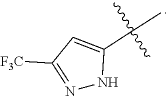 [SMILES: *c1cc(C(F)(F)F)n[nH]1]

In certain embodiments, R² is

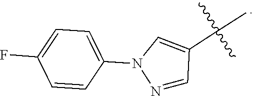 [SMILES: *c1cnn(-c2ccc(F)cc2)c1]

In certain embodiments, R² is

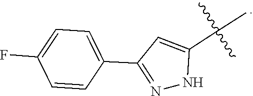 [SMILES: *c1cc(-c2ccc(F)cc2)n[nH]1]

In certain embodiments, R² is

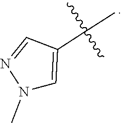 [SMILES: *c1cnn(C)c1]

In certain embodiments, R² is

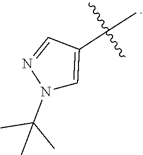 [SMILES: *c1cnn(C(C)(C)C)c1]

In certain embodiments, R² is

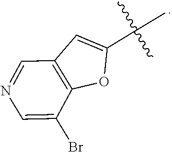 [SMILES: *c1cc2cncc(Br)c2o1]

In certain embodiments, R² is

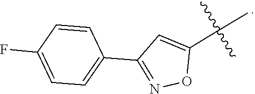 [SMILES: *c1cc(-c2ccc(F)cc2)no1]

In certain embodiments, R² is

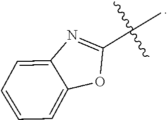 [SMILES: *c1nc2ccccc2o1]

In certain embodiments, R² is

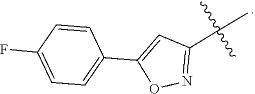 [SMILES: *c1cc(-c2ccc(F)cc2)on1]

In certain embodiments, R² is

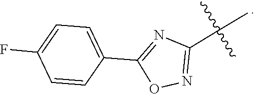 [SMILES: *c1noc(-c2ccc(F)cc2)n1]

In certain embodiments, R² is

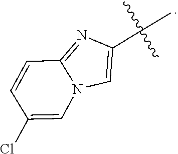 [SMILES: *c1cn2cc(Cl)ccc2n1]

In certain embodiments, R² is

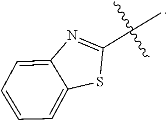 [SMILES: *c1nc2ccccc2s1]

In certain embodiments, R² is

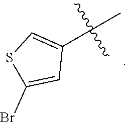 [SMILES: *c1csc(Br)c1]

In certain embodiments, R² is

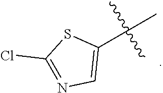 [SMILES: *c1cnc(Cl)s1]

In certain embodiments, R² is

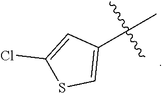 [SMILES: *c1csc(Cl)c1]

In certain embodiments, R² is

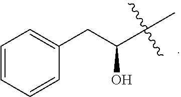 [SMILES: *[C@@H](O)Cc1ccccc1]

In certain embodiments, R² is

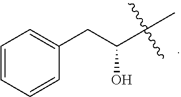 [SMILES: *[C@H](O)Cc1ccccc1]

In certain embodiments, R² is

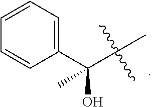 [SMILES: *[C@@](C)(O)c1ccccc1]

In certain embodiments, R² is

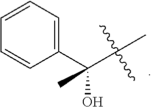 [SMILES: *[C@](C)(O)c1ccccc1]

In certain embodiments, R² is

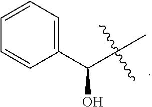 [SMILES: *[C@@H](O)c1ccccc1]

In certain embodiments, R² is

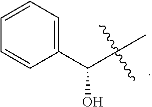 [SMILES: *[C@H](O)c1ccccc1]

In certain embodiments, R² is

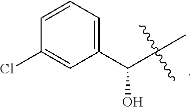 [SMILES: *[C@H](O)c1cccc(Cl)c1]

In certain embodiments, R² is

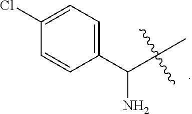 [SMILES: *C(N)c1ccc(Cl)cc1]

In certain embodiments, R² is

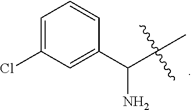 [SMILES: *C(N)c1cccc(Cl)c1]

In certain embodiments, R² is

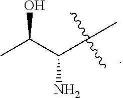 [SMILES: *[C@H](N)[C@@H](C)O]

In certain embodiments, R² is

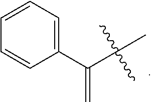 [SMILES: *C(=C)c1ccccc1]

In certain embodiments, R² is

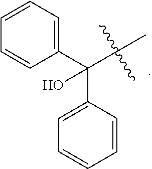 [SMILES: *C(O)(c1ccccc1)c1ccccc1]

In certain embodiments, R² is

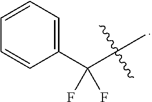 [SMILES: *C(F)(F)c1ccccc1]

In certain embodiments, R² is

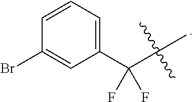 [SMILES: *C(F)(F)c1cccc(Br)c1]

In certain embodiments, R² is

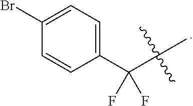 [SMILES: *C(F)(F)c1ccc(Br)cc1]

In certain embodiments, R² is

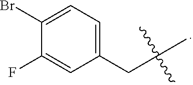 [SMILES: *Cc1ccc(Br)c(F)c1]

In certain embodiments, R² is

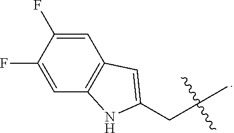 [SMILES: *Cc1cc2cc(F)c(F)cc2[nH]1]

In certain embodiments, R² is

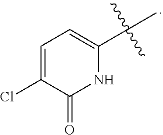 [SMILES: *c1ccc(Cl)c(=O)[nH]1]

In certain embodiments, R² is

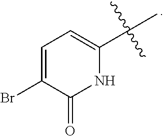 [SMILES: *c1ccc(Br)c(=O)[nH]1]

In certain embodiments, R² is

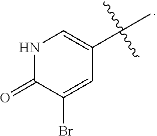 [SMILES: *c1c[nH]c(=O)c(Br)c1]

In certain embodiments, R² is

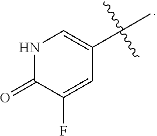 [SMILES: *c1c[nH]c(=O)c(F)c1]

In certain embodiments, R² is

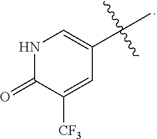 [SMILES: *c1c[nH]c(=O)c(C(F)(F)F)c1]

In certain embodiments, R² is

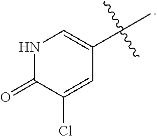 [SMILES: *c1c[nH]c(=O)c(Cl)c1]

In certain embodiments, R² is

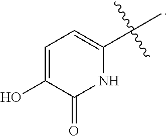 [SMILES: *c1ccc(O)c(=O)[nH]1]

In certain embodiments, R² is

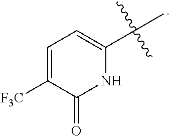 [SMILES: *c1ccc(C(F)(F)F)c(=O)[nH]1]

In certain embodiments, R² is

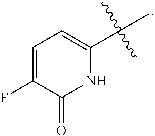 [SMILES: *c1ccc(F)c(=O)[nH]1]

In certain embodiments, R² is

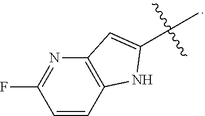 [SMILES: *c1cc2nc(F)ccc2[nH]1]

In certain embodiments, R² is

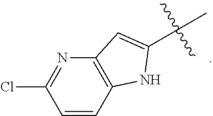 [SMILES: *c1cc2nc(Cl)ccc2[nH]1]

In certain embodiments, R² is

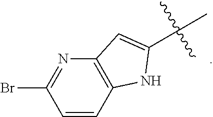 [SMILES: *c1cc2nc(Br)ccc2[nH]1]

In certain embodiments, R² is

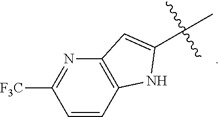 [SMILES: *c1cc2nc(C(F)(F)F)ccc2[nH]1]

In certain embodiments, R² is

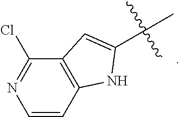 [SMILES: *c1cc2c(Cl)nccc2[nH]1]

In certain embodiments, R² is

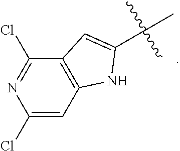 [SMILES: *c1cc2c(Cl)nc(Cl)cc2[nH]1]

In certain embodiments, R² is

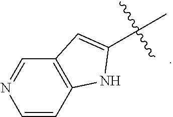 [SMILES: *c1cc2cnccc2[nH]1]

In certain embodiments, R² is

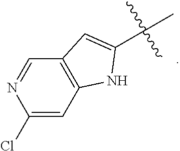 [SMILES: *c1cc2cnc(Cl)cc2[nH]1]

In certain embodiments, R² is

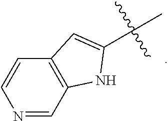 [SMILES: *c1cc2ccncc2[nH]1]

In certain embodiments, R² is

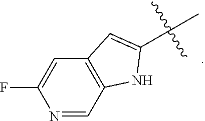 [SMILES: *c1cc2cc(F)ncc2[nH]1]

In certain embodiments, R² is

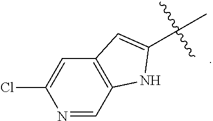 [SMILES: *c1cc2cc(Cl)ncc2[nH]1]

In certain embodiments, R² is

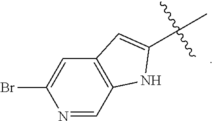 [SMILES: *c1cc2cc(Br)ncc2[nH]1]

In certain embodiments, R² is

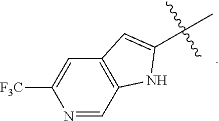 [SMILES: *c1cc2cc(C(F)(F)F)ncc2[nH]1]

In certain embodiments, R² is

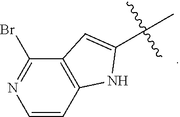 [SMILES: *c1cc2c(Br)nccc2[nH]1]

In certain embodiments, R² is

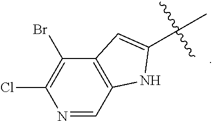 [SMILES: *c1cc2c(Br)c(Cl)ncc2[nH]1]

In certain embodiments, R² is

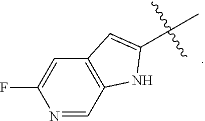 [SMILES: *c1cc2cc(F)ncc2[nH]1]

In certain embodiments, R² is

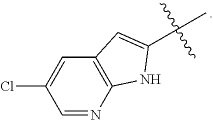 [SMILES: *c1cc2cc(Cl)cnc2[nH]1]

In certain embodiments, R² is

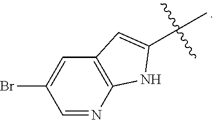 [SMILES: *c1cc2cc(Br)cnc2[nH]1]

In certain embodiments, R² is

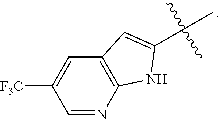 [SMILES: *c1cc2cc(C(F)(F)F)cnc2[nH]1]

In certain embodiments, R² is

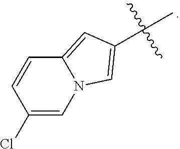 [SMILES: *c1cc2ccc(Cl)cn2c1]

In certain embodiments, R² is

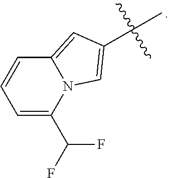 [SMILES: *c1cc2cccc(C(F)F)n2c1]

In certain embodiments, R²

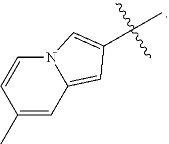 [SMILES: *c1cc2cc(C)ccn2c1]

In certain embodiments, R² is

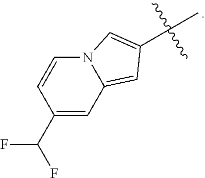 [SMILES: *c1cc2cc(C(F)F)ccn2c1]

In certain embodiments, R² is

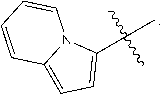 [SMILES: *c1ccc2ccccn12]

In certain embodiments, R² is

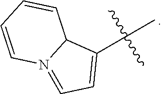

In certain embodiments, R² is

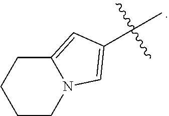 [SMILES: *c1cc2n(c1)CCCC2]

In certain embodiments, R² is

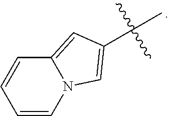 [SMILES: *c1cc2ccccn2c1]

In certain embodiments, R² is

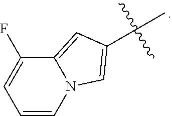 [SMILES: *c1cc2c(F)cccn2c1]

In certain embodiments, R² is

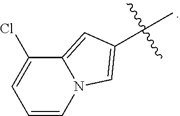 [SMILES: *c1cc2c(Cl)cccn2c1]

In certain embodiments, R² is

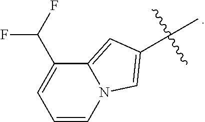 [SMILES: *c1cc2c(C(F)F)cccn2c1]

In certain embodiments, R² is

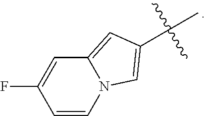 [SMILES: *c1cc2cc(F)ccn2c1]

In certain embodiments, R² is

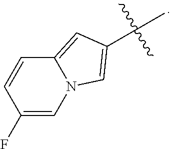 [SMILES: *c1cc2ccc(F)cn2c1]

In certain embodiments, R² is

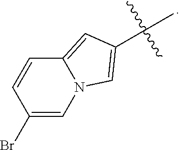 [SMILES: *c1cc2ccc(Br)cn2c1]

In certain embodiments, R² is

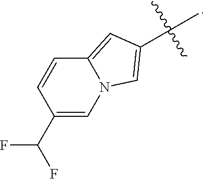 [SMILES: *c1cc2ccc(C(F)F)cn2c1]

In certain embodiments, R² is

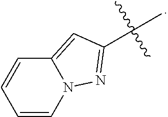 [SMILES: *c1cc2ccccn2n1]

In certain embodiments, R² is

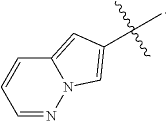 [SMILES: *c1cc2cccnn2c1]

In certain embodiments, R² is

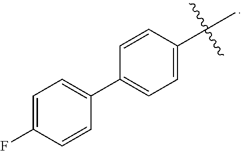 [SMILES: *c1ccc(-c2ccc(F)cc2)cc1]

In certain embodiments, R² is

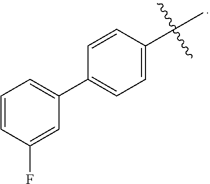 [SMILES: *c1ccc(-c2cccc(F)c2)cc1]

In certain embodiments, R² is

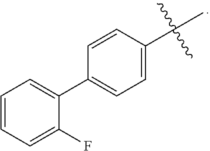 [SMILES: *c1ccc(-c2ccccc2F)cc1]

In certain embodiments, R² is

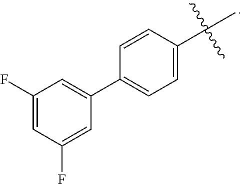 [SMILES: *c1ccc(-c2cc(F)cc(F)c2)cc1]

In certain embodiments, R² is

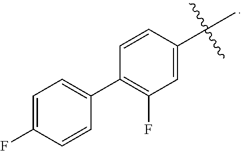 [SMILES: *c1ccc(-c2ccc(F)cc2)c(F)c1]

In certain embodiments, R² is

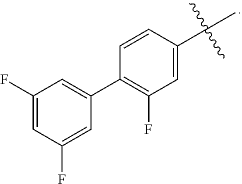 [SMILES: *c1ccc(-c2cc(F)cc(F)c2)c(F)c1]

In certain embodiments, R² is

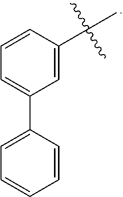 [SMILES: *c1cccc(-c2ccccc2)c1]

In certain embodiments, R² is

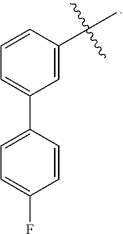 [SMILES: *c1cccc(-c2ccc(F)cc2)c1]

In certain embodiments, R² is

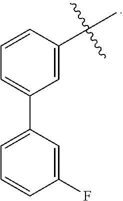 [SMILES: *c1cccc(-c2cccc(F)c2)c1]

In certain embodiments, R² is

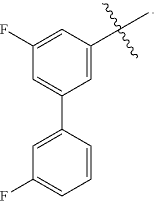 [SMILES: *c1cc(F)cc(-c2cccc(F)c2)c1]

In certain embodiments, R² is

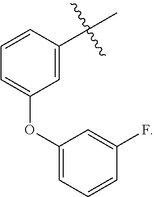 [SMILES: *c1cccc(Oc2cccc(F)c2)c1]

In certain embodiments, R² is

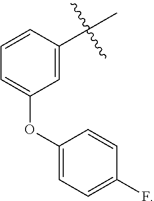 [SMILES: *c1cccc(Oc2ccc(F)cc2)c1]

In certain embodiments, R² is

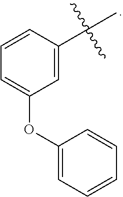 [SMILES: *c1cccc(Oc2ccccc2)c1]

In certain embodiments, R² is

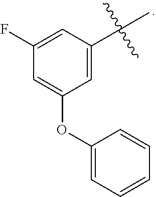 [SMILES: *c1cc(F)cc(Oc2ccccc2)c1]

In certain embodiments, R² is

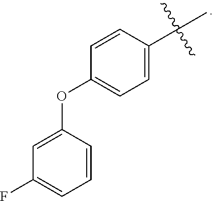 [SMILES: *c1ccc(Oc2cccc(F)c2)cc1]

In certain embodiments, R² is

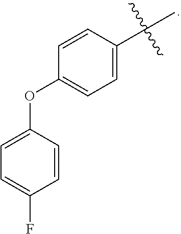 [SMILES: *c1ccc(Oc2ccc(F)cc2)cc1]

In certain embodiments, R² is

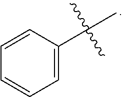 [SMILES: *c1ccccc1]

In certain embodiments, R² is

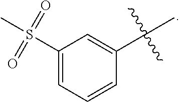 [SMILES: *c1cccc(S(C)(=O)=O)c1]

In certain embodiments, R² is

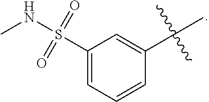 [SMILES: *c1cccc(S(=O)(=O)NC)c1]

In certain embodiments, R² is

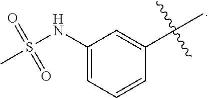 [SMILES: *c1cccc(NS(C)(=O)=O)c1]

In certain embodiments, R² is

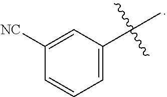 [SMILES: *c1cccc(C#N)c1]

In certain embodiments, R² is

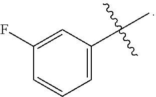 [SMILES: *c1cccc(F)c1]

In certain embodiments, R² is

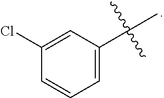 [SMILES: *c1cccc(Cl)c1]

In certain embodiments, R² is

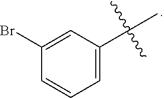 [SMILES: *c1cccc(Br)c1]

In certain embodiments, R² is

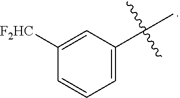 [SMILES: *c1cccc(C(F)F)c1]

In certain embodiments, R² is

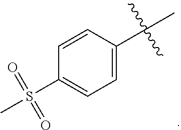 [SMILES: *c1ccc(S(C)(=O)=O)cc1]

In certain embodiments, R² is

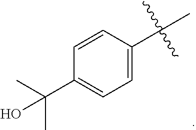 [SMILES: *c1ccc(C(C)(C)O)cc1]

In certain embodiments, R² is

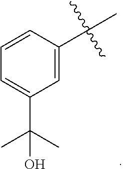 [SMILES: *c1cccc(C(C)(C)O)c1]

In certain embodiments, R² is

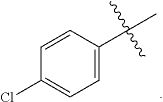 [SMILES: *c1ccc(Cl)cc1]

In certain embodiments, R² is

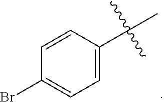 [SMILES: *c1ccc(Br)cc1]

In certain embodiments, R² is

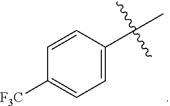 [SMILES: *c1ccc(C(F)(F)F)cc1]

In certain embodiments, R² is

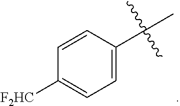 [SMILES: *c1ccc(C(F)F)cc1]

In certain embodiments, R² is

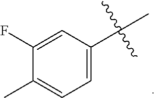 [SMILES: *c1ccc(C)c(F)c1]

In certain embodiments, R² is

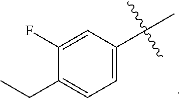 [SMILES: *c1ccc(CC)c(F)c1]

In certain embodiments, R² is

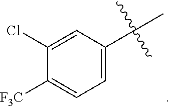 [SMILES: *c1ccc(C(F)(F)F)c(Cl)c1]

In certain embodiments, R² is

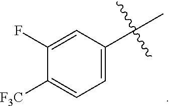 [SMILES: *c1ccc(C(F)(F)F)c(F)c1]

In certain embodiments, R² is

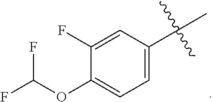 [SMILES: *c1ccc(OC(F)F)c(F)c1]

In certain embodiments, R² is

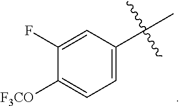 [SMILES: *c1ccc(OC(F)(F)F)c(F)c1]

In certain embodiments, R² is

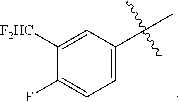 [SMILES: *c1ccc(F)c(C(F)F)c1]

In certain embodiments, R² is

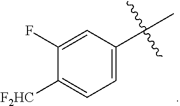 [SMILES: *c1ccc(C(F)F)c(F)c1]

In certain embodiments, R² is

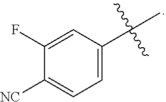 [SMILES: *c1ccc(C#N)c(F)c1]

In certain embodiments, R² is

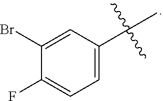 [SMILES: *c1ccc(F)c(Br)c1]

In certain embodiments, R² is

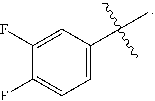 [SMILES: *c1ccc(F)c(F)c1]

In certain embodiments, R² is

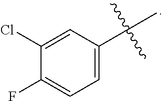 [SMILES: *c1ccc(F)c(Cl)c1]

In certain embodiments, R² is

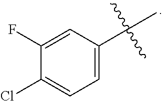 [SMILES: *c1ccc(Cl)c(F)c1]

In certain embodiments, R² is

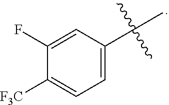 [SMILES: *c1ccc(C(F)(F)F)c(F)c1]

In certain embodiments, R² is

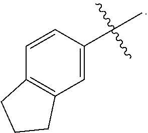 [SMILES: *c1ccc2c(c1)CCC2]

In certain embodiments, R² is

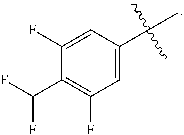 [SMILES: *c1cc(F)c(C(F)F)c(F)c1]

In certain embodiments, R² is

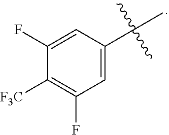 [SMILES: *c1cc(F)c(C(F)(F)F)c(F)c1]

In certain embodiments, R² is

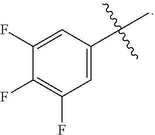 [SMILES: *c1cc(F)c(F)c(F)c1]

In certain embodiments, R² is

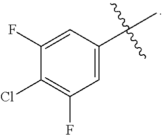 [SMILES: *c1cc(F)c(Cl)c(F)c1]

In certain embodiments, R² is

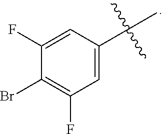 [SMILES: *c1cc(F)c(Br)c(F)c1]

In certain embodiments, R² is

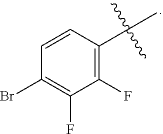 [SMILES: *c1ccc(Br)c(F)c1F]

In certain embodiments, R² is

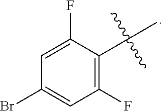 [SMILES: *c1c(F)cc(Br)cc1F]

In certain embodiments, R² is

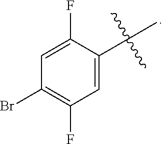 [SMILES: *c1cc(F)c(Br)cc1F]

In certain embodiments, R² is

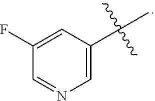 [SMILES: *c1cncc(F)c1]

In certain embodiments, R² is

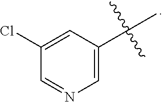 [SMILES: *c1cncc(Cl)c1]

In certain embodiments, R² is

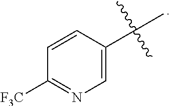 [SMILES: *c1ccc(C(F)(F)F)nc1]

In certain embodiments, R² is

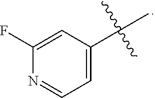 [SMILES: *c1ccnc(F)c1]

In certain embodiments, R² is

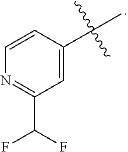 [SMILES: *c1ccnc(C(F)F)c1]

In certain embodiments, R² is

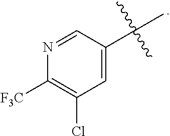 [SMILES: *c1cnc(C(F)(F)F)c(Cl)c1]

In certain embodiments, R² is

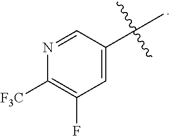 [SMILES: *c1cnc(C(F)(F)F)c(F)c1]

In certain embodiments, R² is

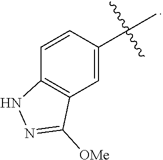 [SMILES: *c1ccc2[nH]nc(OC)c2c1]

In certain embodiments, R² is

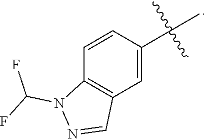 [SMILES: *c1ccc2c(cnn2C(F)F)c1]

In certain embodiments, R² is

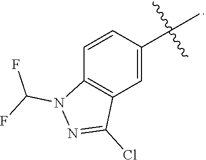 [SMILES: *c1ccc2c(c1)c(Cl)nn2C(F)F]

In certain embodiments R² is

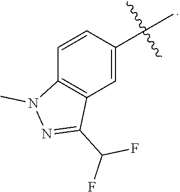 [SMILES: *c1ccc2c(c1)c(C(F)F)nn2C]

In certain embodiments, R² is

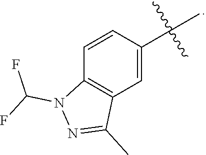 [SMILES: *c1ccc2c(c1)c(C)nn2C(F)F]

In certain embodiments, R² is

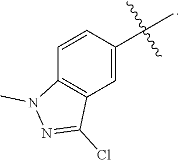 [SMILES: *c1ccc2c(c1)c(Cl)nn2C]

In certain embodiments, R² is

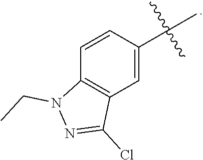 [SMILES: *c1ccc2c(c1)c(Cl)nn2CC]

In certain embodiments, R² is

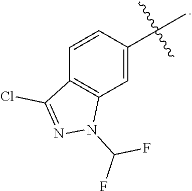 [SMILES: *c1ccc2c(Cl)nn(C(F)F)c2c1]

In certain embodiments, R² is

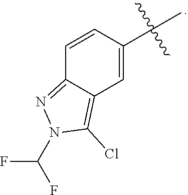 [SMILES: *c1ccc2nn(C(F)F)c(Cl)c2c1]

In certain embodiments, R² is

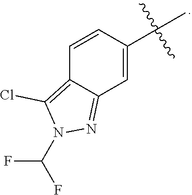 [SMILES: *c1ccc2c(Cl)n(C(F)F)nc2c1]

In certain embodiments, R² is

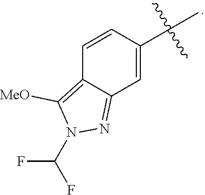 [SMILES: *c1ccc2c(OC)n(C(F)F)nc2c1]

In certain embodiments, R² is

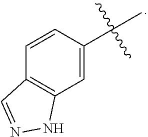 [SMILES: *c1ccc2cn[nH]c2c1]

In certain embodiments, R² is

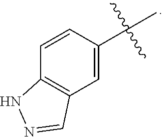 [SMILES: *c1ccc2[nH]ncc2c1]

In certain embodiments, R² is

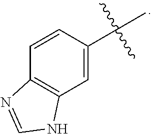 [SMILES: *c1ccc2nc[nH]c2c1]

In certain embodiments, R² is

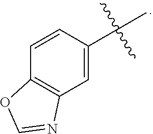 [SMILES: *c1ccc2ocnc2c1]

In certain embodiments, R² is

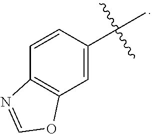 [SMILES: *c1ccc2ncoc2c1]

In certain embodiments, R² is

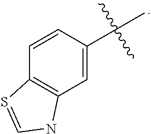

In certain embodiments, R² is

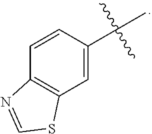 [SMILES: *c1ccc2ncsc2c1]

In certain embodiments, R² is

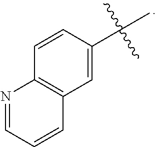 [SMILES: *c1ccc2ncccc2c1]

In certain embodiments, R² is

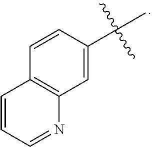 [SMILES: *c1ccc2cccnc2c1]

In certain embodiments, R² is

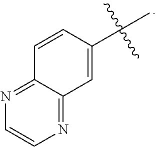 [SMILES: *c1ccc2nccnc2c1]

In certain embodiments, R² is

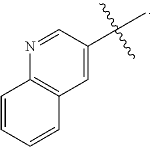 [SMILES: *c1cnc2ccccc2c1]

In certain embodiments, R² is

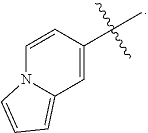 [SMILES: *c1ccn2cccc2c1]

In certain embodiments, R² is

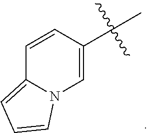 [SMILES: *c1ccc2cccn2c1]

In certain embodiments, R² is

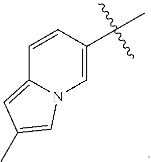 [SMILES: *c1ccc2cc(C)cn2c1]

In certain embodiments, R² is

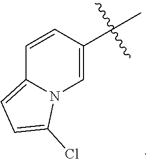 [SMILES: *c1ccc2ccc(Cl)n2c1]

In certain embodiments, R² is

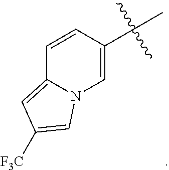 [SMILES: *c1ccc2cc(C(F)(F)F)cn2c1]

In certain embodiments, R² is

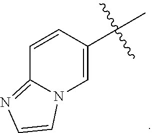 [SMILES: *c1ccc2nccn2c1]

In certain embodiments, R² is

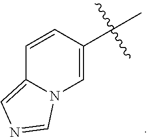 [SMILES: *c1ccc2cncn2c1]

In certain embodiments, R² is

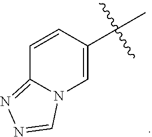 [SMILES: *c1ccc2nncn2c1]

In certain embodiments, R^{3a} is H. In certain embodiments, R^{3a} is methyl.

In certain embodiments, R^{3b} is H. In certain embodiments, R^{3b} is methyl.

In certain embodiments, R⁵ is selected from the group consisting of H, methyl, ethyl, isopropyl, n-propyl, cyclopropyl, n-butyl, isobutyl, sec-butyl, tert-butyl, cyclobutyl, isopropylmethyl, —(CH₂)₂₋₆OH, —(CH₂)₂₋₆O(C₁-C₆ alkyl), ¹³CD₃, optionally substituted benzyl, and optionally substituted phenyl.

In certain embodiments, R⁶ is selected from the group consisting of H, D, and methyl. In certain embodiments, R⁶ is H. In certain embodiments, R⁶ is D. In certain embodiments, R⁶ is methyl.

In certain embodiments, p is independently 1 or 2, when Q is —O—, —S—, —S(O)—, —S(O)₂—, or —NR¹¹.

In certain embodiments, R⁷ is a divalent group selected from the group consisting of —CH₂CH₂—, —CH₂CH₂CH₂—, —CH₂OCH₂—, —CH₂CH₂CH₂CH₂—, —CH₂OCH₂CH₂—, and —CH₂CH₂OCH₂—, wherein each CH₂ group is optionally substituted with one or two CH₃ groups.

In certain embodiments, R⁷ is a divalent group selected from the group consisting of —CH₂CH₂—, —CH(CH₃)CH₂—, —CH₂CH(CH₃)—, —C(CH₃)₂CH₂—, —CH₂C(CH₃)₂—, —CH(CH₃)CH(CH₃)—, —CH(CH₃)C(CH₃)₂—, —C(CH₃)₂CH(CH₃)—, and —C(CH₃)₂C(CH₃)₂—.

In certain embodiments, R⁷ is a divalent group selected from the group consisting of —CH₂OCH₂—, —CH(CH₃)OCH₂—, —CH₂OCH(CH₃)—, —CH(CH₃)OCH(CH₃)—, —C(CH₃)₂OCH₂—, —CH₂OC(CH₃)₂—, —C(CH₃)₂OCH(CH₃)—, —CH(CH₃)OC(CH₃)₂—, and C(CH₃)₂OC(CH₃)₂—.

In certain embodiments, R⁷ is a divalent group selected from the group consisting of —CH₂CH₂CH₂—, —CH(CH₃)CH₂CH₂—, —CH₂CH(CH₃)CH₂—, —CH₂CH₂CH(CH₃)—, —CH(CH₃)CH(CH₃)CH₂—, —CH(CH₃)CH₂CH(CH₃)—, —CH₂CH(CH₃)CH(CH₃)—, —C(CH₃)₂CH₂CH₂—, —CH₂C(CH₃)₂CH₂—, —CH₂CH₂C(CH₃)₂—, —CH(CH₃)CH(CH₃)CH(CH₃)—, —C(CH₃)₂CH(CH₃)CH₂—, —C(CH₃)₂CH₂CH(CH₃)—, —CH(CH₃)C(CH₃)₂CH₂—, —CH₂C(CH₃)₂CH(CH₃)—, —CH(CH₃)CH₂C(CH₃)₂—, —CH₂CH(CH₃)C(CH₃)₂—, —C(CH₃)₂CH(CH₃)CH(CH₃)—, —C(CH₃)₂C(CH₃)₂CH₂—, —C(CH₃)₂CH₂C(CH₃)₂—, —CH(CH₃)C(CH₃)₂CH(CH₃)—, CH₂C(CH₃)₂C(CH₃)₂—, —CH(CH₃)CH(CH₃)C(CH₃)₂—, —CH(CH₃)C(CH₃)₂C(CH₃)₂—, —C(CH₃)₂CH(CH₃)C(CH₃)₂—, —C(CH₃)₂C(CH₃)₂CH(CH₃)—, and —C(CH₃)₂C(CH₃)₂C(CH₃)₂—.

In certain embodiments, R⁷ is a divalent group selected from the group consisting of —CH₂OCH₂CH₂—, —CH(CH₃)OCH₂CH₂—, —CH₂OCH(CH₃)CH₂—, —CH₂OCH₂CH(CH₃)—, —CH(CH₃)OCH(CH₃)CH₂—, —CH(CH₃)OCH₂CH(CH₃)—, —CH₂OCH(CH₃)CH(CH₃)—, —C(CH₃)₂OCH₂CH₂—, —CH₂OC(CH₃)₂CH₂—, —CH₂OCH₂C(CH₃)₂—, —CH(CH₃)OCH(CH₃)CH(CH₃)—, —C(CH₃)₂OCH(CH₃)CH₂—, —C(CH₃)₂OCH₂CH(CH₃)—, —CH(CH₃)OC(CH₃)₂CH₂—, —CH₂OC(CH₃)₂CH(CH₃)—, —CH(CH₃)OCH₂C(CH₃)₂—, —CH₂OCH(CH₃)C(CH₃)₂—, —C(CH₃)₂OCH(CH₃)CH(CH₃)—, —C(CH₃)₂OC(CH₃)₂CH₂—, —C(CH₃)₂OCH₂C(CH₃)₂—, —CH(CH₃)OC(CH₃)₂CH(CH₃)—, —CH₂OC(CH₃)₂C(CH₃)₂—, —CH(CH₃)OCH(CH₃)C(CH₃)₂—, —CH(CH₃)OC(CH₃)₂C(CH₃)₂—, —C(CH₃)₂OCH(CH₃)C(CH₃)₂—, —C(CH₃)₂OC(CH₃)₂CH(CH₃)—, and —C(CH₃)₂OC(CH₃)₂C(CH₃)₂—.

In certain embodiments, R⁷ is a divalent group selected from the group consisting of —CH₂CH₂OCH₂—, —CH(CH₃)CH₂OCH₂—, —CH₂CH(CH₃)OCH₂—, —CH₂CH₂OCH(CH₃)—, —CH(CH₃)CH(CH₃)OCH₂—, —CH(CH₃)CH₂OCH(CH₃)—, —CH₂CH(CH₃)OCH(CH₃)—, —C(CH₃)₂CH₂OCH₂—, —CH₂C(CH₃)₂OCH₂—, —CH₂CH₂OC(CH₃)₂—, —CH(CH₃) CH(CH₃)OCH(CH₃)—, —C(CH₃)₂CH(CH₃)OCH₂—, —C(CH₃)₂CH₂OCH(CH₃)—, —CH(CH₃)C(CH₃)₂OCH₂—, —CH₂C(CH₃)₂OCH(CH₃)—, —CH(CH₃)CH₂OC(CH₃)₂—, —CH₂CH(CH₃)OC(CH₃)₂—, —C(CH₃)₂CH(CH₃)OCH(CH₃)—, —C(CH₃)₂C(CH₃)₂OCH₂—, —C(CH₃)₂CH₂OC(CH₃)₂—, —CH(CH₃)C(CH₃)₂OCH(CH₃)—, —CH₂C(CH₃)₂OC(CH₃)₂—, —CH(CH₃)CH(CH₃)OC(CH₃)₂—, —CH(CH₃)C(CH₃)₂OC(CH₃)₂—, —C(CH₃)₂CH(CH₃)OC(CH₃)₂—, —C(CH₃)₂C(CH₃)₂OCH(CH₃)—, and —C(CH₃)₂C(CH₃)₂OC(CH₃)₂—.

In certain embodiments, R⁷ is a divalent group selected from the group consisting of —CH₂N(CH₃)CH₂—, —CH₂N(CH₃)CH(CH₃)—, —CH₂N(CH₃)C(CH₃)₂—, —CH(CH₃)N(CH₃)CH₂—, —CH(CH₃)N(CH₃)CH(CH₃)—, —CH(CH₃)N(CH₃)C(CH₃)₂—, —C(CH₃)₂N(CH₃)CH₂—, —C(CH₃)₂N(CH₃)CH(CH₃)—, —C(CH₃)₂N(CH₃)C(CH₃)₂—, —CH₂NHCH₂—, —CH₂NHCH(CH₃)—, —CH₂NHC(CH₃)₂—, —CH(CH₃)NHCH₂—, —CH(CH₃)NHCH(CH₃)—, —CH(CH₃)NHC(CH₃)₂—, —C(CH₃)₂NHCH₂—, —C(CH₃)₂NHCH(CH₃)—, and —C(CH₃)₂NHC(CH₃)₂—.

In certain embodiments, R⁷ is a divalent group selected from the group consisting of —CH₂N((C═O)CH₃)CH₂— and —CH₂N(CH₂CH₂OH)CH₂—.

In certain embodiments, R⁷ is a divalent group selected from the group consisting of —CH₂CH₂CH(OH)—, —CH₂CH(OH)CH₂—, and —CH(OH)CH₂CH₂—.

In certain embodiments, R⁷ is a divalent group selected from the group consisting of —CH(OH)OCH₂—, —CH₂OCH(OH)—, —CH(OCH₃)OCH₂—, —CH₂OCH(OCH₃)—, —CH(O(C═O)CH(CH₃)₂)OCH₂—, —CH₂OCH(O(C═O)CH(CH₃)₂)—, —CH(O(C═O)CH(CH₃CH₂)₂)OCH₂—, and —CH₂OCH(O(C═O)CH(CH₃CH₂)₂)—.

In certain embodiments, R⁷ is a divalent group selected from the group consisting of —CH₂OCHF—, —CH₂OCF₂—, —CHFOCH₂—, —CHFOCHF—, —CHFOCF₂—, —CF2OCH₂—, —CF2OCHF—, and —CF2OCF₂—.

In certain embodiments, R⁷ is a divalent group selected from the group consisting of —CH₂S(═O)CH₂— and —CH₂S(═O)₂CH₂—.

In certain embodiments, R⁸ is H. In other embodiments, R⁸ is methyl. In yet other embodiments, R⁸ is ethyl. In yet other embodiments, R⁸ is 1-(2,2,2-trifluoroethyl). In yet other embodiments, R⁸ is 1-propyl. In yet other embodiments, R⁸ is isopropyl. In yet other embodiments, R⁸ is cyclopropyl. In yet other embodiments, R⁸ is 1-(2-hydroxy)ethyl. In yet other embodiments, R⁸ is 1-(2-methoxy)ethyl. In yet other embodiments, R⁸ is 1-(3-hydroxy)propyl. In yet other embodiments, R⁸ is 1-(3-methoxy)propyl. In yet other embodiments, R⁸ is triazolylmethyl. In yet other embodiments, R⁸ is 2-hydroxyethyl. In yet other embodiments, R⁸ is 2-aminoethyl.

In certain embodiments, R¹¹ is H. In other embodiments, R¹¹ is methoxy. In yet other embodiments, R¹¹ is ethoxy. In yet other embodiments, R¹¹ is methyl. In yet other embodiments, R¹¹ is ethyl. In yet other embodiments, R¹¹ is 2-hydroxyethoxy. In yet other embodiments, R¹¹ is amino. In yet other embodiments, R¹¹ is methylamino. In yet other embodiments, R¹¹ is ethylamino. In yet other embodiments, R¹¹ is dimethylamino. In yet other embodiments, R¹¹ is (2-hydroxyethyl)amino. In yet other embodiments, R¹¹ is (2-aminoethyl)amino. In yet other embodiments, R¹¹ is triazolyl. In yet other embodiments, R¹¹ is triazolylmethoxy. In yet other embodiments, R¹¹ is (N-methyltriazolyl)methyl. In yet other embodiments, R¹¹ is triazolylmethylamino. In yet other embodiments, R¹¹ is (N-methyltriazolyl)methylamino. In yet other embodiments, R¹¹ is CN. In yet other embodiments, R¹¹ is hydroxymethyl. In yet other embodiments, R¹¹ is carboxy. In yet other embodiments, R¹¹ is aminocarbonyl. In yet other embodiments, R¹¹ is methylaminocarbonyl. In yet other embodiments, R¹¹ is dimethylaminocarbonyl. In yet other embodiments, R¹¹ is methylsulfonyl. In yet other embodiments, R¹¹ is pyridylmethoxy. In yet other embodiments, R¹¹ is (2-aminoethyl)hydroxy.

In certain embodiments, the compound is any compound disclosed herein, or a salt, solvate, prodrug, isotopically labelled, stereoisomer, any mixture of stereoisomers, tautomer, and/or any mixture of tautomers thereof.

In certain embodiments, the compound is at least one selected from Table 1, or a salt, solvate, prodrug, isotopically labelled, stereoisomer, any mixture of stereoisomers, tautomer, and/or any mixture of tautomers thereof.

In certain embodiments, the compound is:
N-(8-fluoro-6-oxo-1,4,5,6-tetrahydro-2H-pyrano[3,4-c]isoquinolin-1-yl)-N-methyl-1H-indole-2-carboxamide;
N-(8-fluoro-6-oxo-1,4,5,6-tetrahydro-2H-pyrano[3,4-c]isoquinolin-1-yl)-N-methylindoline-2-carboxamide;
N-(8,9-difluoro-6-oxo-1,4,5,6-tetrahydro-2H-pyrano[3,4-c]isoquinolin-1-yl)-5-fluoro-N-methylindoline-2-carboxamide;
N-(8,9-difluoro-6-oxo-1,4,5,6-tetrahydro-2H-pyrano[3,4-c]isoquinolin-1-yl)-8-fluoro-N-methylindolizine-2-carboxamide;
N-(8,9-difluoro-6-oxo-1,4,5,6-tetrahydro-2H-pyrano[3,4-c]isoquinolin-1-yl)-N-methylindolizine-2-carboxamide;
N-(8,9-difluoro-6-oxo-1,4,5,6-tetrahydro-2H-pyrano[3,4-c]isoquinolin-1-yl)-4,6-difluoro-N-methylindoline-2-carboxamide;
8-chloro-N-(8,9-difluoro-6-oxo-1,4,5,6-tetrahydro-2H-pyrano[3,4-c]isoquinolin-1-yl)-N-methylindolizine-2-carboxamide;
4-fluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-4-fluoro-N-methyl-1H-indole-2-carboxamide;
N-(8,9-difluoro-6-oxo-1,4,5,6-tetrahydro-2H-pyrano[3,4-c]isoquinolin-1-yl)-6-fluoro-N-methyl-1H-indole-2-carboxamide;
N-(8,9-difluoro-6-oxo-1,4,5,6-tetrahydro-2H-pyrano[3,4-c]isoquinolin-1-yl)-5-fluoro-N-methyl-1H-indole-2-carboxamide;
5-fluoro-N-(8-fluoro-6-oxo-1,4,5,6-tetrahydro-2H-pyrano[3,4-c]isoquinolin-1-yl)-N-methyl-1H-indole-2-carboxamide;
6-fluoro-N-(8-fluoro-6-oxo-1,4,5,6-tetrahydro-2H-pyrano[3,4-c]isoquinolin-1-yl)-N-methyl-1H-indole-2-carboxamide;
7-fluoro-N-(8-fluoro-6-oxo-1,4,5,6-tetrahydro-2H-pyrano[3,4-c]isoquinolin-1-yl)-N-methyl-1H-indole-2-carboxamide;
4,6-difluoro-N-(8-fluoro-6-oxo-1,4,5,6-tetrahydro-2H-pyrano[3,4-c]isoquinolin-1-yl)-N-methyl-1H-indole-2-carboxamide;
4,5-difluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-7-fluoro-N-methyl-1H-indole-2-carboxamide;
N-(8,9-difluoro-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-4,6-difluoro-N-methyl-1H-indole-2-carboxamide;
N-(8,9-difluoro-6-oxo-1,4,5,6-tetrahydro-2H-pyrano[3,4-c]isoquinolin-1-yl)-4,5-difluoro-N-methyl-1H-indole-2-carboxamide;
N-(8,9-difluoro-6-oxo-1,4,5,6-tetrahydro-2H-pyrano[3,4-c]isoquinolin-1-yl)-5,5-difluoro-N-methyl-4,5,6,7-tetrahydro-1H-indole-2-carboxamide;
5,6-difluoro-N-(8-fluoro-6-oxo-1,4,5,6-tetrahydro-2H-pyrano[3,4-c]isoquinolin-1-yl)-N-methyl-1H-indole-2-carboxamide;
6-chloro-N-(8,9-difluoro-6-oxo-1,4,5,6-tetrahydro-2H-pyrano[3,4-c]isoquinolin-1-yl)-N-methylimidazo[1,2-a]pyridine-2-carboxamide;
6-chloro-N-(8-fluoro-6-oxo-1,4,5,6-tetrahydro-2H-pyrano[3,4-c]isoquinolin-1-yl)-N-methylimidazo[1,2-a]pyridine-2-carboxamide;
6-chloro-N-(8-fluoro-6-oxo-1,4,5,6-tetrahydro-2H-pyrano[3,4-c]isoquinolin-1-yl)-N-ethylimidazo[1,2-a]pyridine-2-carboxamide;
N-(8,9-difluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N,3,3-trimethylindoline-2-carboxamide;
N-(8-fluoro-3-methyl-6-oxo-1,2,3,4,5,6-hexahydrobenzo[c][1,7]naphthyridin-1-yl)-N-methyl-1H-indole-2-carboxamide;
N-(8,9-difluoro-3-methyl-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methylpyrazolo[1,5-a]pyridine-2-carboxamide;
N-(8,9-difluoro-6-oxo-1,4,5,6-tetrahydro-2H-pyrano[3,4-c]isoquinolin-1-yl)-N-methylpyrrolo[1,2-b]pyridazine-6-carboxamide;
N-(8,9-difluoro-6-oxo-1,4,5,6-tetrahydro-2H-pyrano[3,4-c]isoquinolin-1-yl)-N-methylquinoline-7-carboxamide;
N-(8,9-difluoro-6-oxo-1,4,5,6-tetrahydro-2H-pyrano[3,4-c]isoquinolin-1-yl)-N-methylquinoline-6-carboxamide;
N-(3-Acetyl-8-fluoro-6-oxo-1,2,3,4,5,6-hexahydrobenzo[c][1,7]naphthyridin-1-yl)-N-methyl-1H-indole-2-carboxamide;
N-(8,9-Difluoro-6-oxo-1,4,5,6-tetrahydro-2H-pyrano[3,4-c]isoquinolin-1-yl)-N-methyl-[1,2,4]triazolo[4,3-a]pyridine-6-carboxamide;
N-(8,9-Difluoro-6-oxo-1,4,5,6-tetrahydro-2H-pyrano[3,4-c]isoquinolin-1-yl)-N-methylquinoline-3-carboxamide;
N-(8,9-Difluoro-6-oxo-1,4,5,6-tetrahydro-2H-pyrano[3,4-c]isoquinolin-1-yl)-N-methylquinoxaline-6-carboxamide;
N-(8,9-Difluoro-6-oxo-1,4,5,6-tetrahydro-2H-pyrano[3,4-c]isoquinolin-1-yl)-N-methyl-6-(trifluoromethyl)nicotinamide;
N-(8,9-Difluoro-6-oxo-1,4,5,6-tetrahydro-2H-pyrano[3,4-c]isoquinolin-1-yl)-3-fluoro-N-methyl-4-(trifluoromethyl)benzamide;
4-Chloro-N-(8,9-difluoro-6-oxo-1,4,5,6-tetrahydro-2H-pyrano[3,4-c]isoquinolin-1-yl)-3-fluoro-N-methylbenzamide;
N-(8,9-Difluoro-6-oxo-1,4,5,6-tetrahydro-2H-pyrano[3,4-c]isoquinolin-1-yl)-3,4,5-trifluoro-N-methylbenzamide;
N-(8-Fluoro-6-oxo-1,2,3,4,5,6-hexahydrobenzo[c][1,7]naphthyridin-1-yl)-N-methyl-1H-indole-2-carboxamide;
N-(8-Fluoro-3-(2-hydroxyethyl)-6-oxo-1,2,3,4,5,6-hexahydrobenzo[c][1,7]naphthyridin-1-yl)-N-methyl-1H-indole-2-carboxamide;
N-(3-Acetyl-8,9-difluoro-6-oxo-1,2,3,4,5,6-hexahydrobenzo[c][1,7]naphthyridin-1-yl)-N-methyl-1H-indole-2-carboxamide;
N-(8,9-Difluoro-6-oxo-1,4,5,6-tetrahydro-2H-pyrano[3,4-c]isoquinolin-1-yl)-5-fluoro-N-methylnicotinamide;
5-Chloro-N-(8,9-difluoro-6-oxo-1,4,5,6-tetrahydro-2H-pyrano[3,4-c]isoquinolin-1-yl)-N-methylnicotinamide;
N-(8,9-Difluoro-6-oxo-1,4,5,6-tetrahydro-2H-pyrano[3,4-c]isoquinolin-1-yl)-2-fluoro-N-methylisonicotinamide;
N-(8,9-Difluoro-6-oxo-1,4,5,6-tetrahydro-2H-pyrano[3,4-c]isoquinolin-1-yl)-3-(difluoromethyl)-N-methylbenzamide;
N-(8,9-Difluoro-6-oxo-1,4,5,6-tetrahydro-2H-pyrano[3,4-c]isoquinolin-1-yl)-2-hydroxy-N-methyl-3-phenylpropanamide;
N-(8,9-Difluoro-3-(2-hydroxyethyl)-6-oxo-1,2,3,4,5,6-hexahydrobenzo[c][1,7]naphthyridin-1-yl)-N-methyl-1H-indole-2-carboxamide;
N-(8,9-Difluoro-6-oxo-1,4,5,6-tetrahydro-2H-pyrano[3,4-c]isoquinolin-1-yl)-N-methylbenzamide;
N-(8,9-Difluoro-6-oxo-1,4,5,6-tetrahydro-2H-pyrano[3,4-c]isoquinolin-1-yl)-3,4-difluoro-N-methylbenzamide;
N-(8,9-Difluoro-6-oxo-1,4,5,6-tetrahydro-2H-pyrano[3,4-c]isoquinolin-1-yl)-2-hydroxy-N-methyl-2-phenylpropanamide;
N-(8,9-Difluoro-6-oxo-1,4,5,6-tetrahydro-2H-pyrano[3,4-c]isoquinolin-1-yl)-2-hydroxy-N-methyl-2-phenylacetamide;
N-(8,9-Difluoro-6-oxo-1,4,5,6-tetrahydro-2H-pyrano[3,4-c]isoquinolin-1-yl)-4,5,6-trifluoro-N-methyl-1H-indole-2-carboxamide;
N-(8,9-Difluoro-6-oxo-1,4,5,6-tetrahydro-2H-pyrano[3,4-c]isoquinolin-1-yl)-N-methyl-4-(trifluoromethyl)benzamide;
4-Chloro-N-(8,9-difluoro-6-oxo-1,4,5,6-tetrahydro-2H-pyrano[3,4-c]isoquinolin-1-yl)-N-methylbenzamide;
4-Cyano-N-(8,9-difluoro-6-oxo-1,4,5,6-tetrahydro-2H-pyrano[3,4-c]isoquinolin-1-yl)-3-fluoro-N-methylbenzamide;
N-(8,9-Difluoro-6-oxo-1,2,3,4,5,6-hexahydrobenzo[c][1,7]naphthyridin-1-yl)-5-fluoro-N-methyl-1H-indole-2-carboxamide;
N-(8,9-Difluoro-6-oxo-1,4,5,6-tetrahydro-2H-thiopyrano[3,4-c]isoquinolin-1-yl)-8-fluoro-N-methylindolizine-2-carboxamide;
N-(8,9-Difluoro-6-oxo-1,2,3,4,5,6-hexahydrobenzo[c][1,7]naphthyridin-1-yl)-4-fluoro-N-methyl-1H-indole-2-carboxamide;
N-(8,9-Difluoro-6-oxo-1,2,3,4,5,6-hexahydrobenzo[c][1,7]naphthyridin-1-yl)-6-fluoro-N-methyl-1H-indole-2-carboxamide;
N-(8,9-Difluoro-6-oxo-1,4,5,6-tetrahydro-2H-pyrano[3,4-c]isoquinolin-1-yl)-3-(difluoromethyl)-4-fluoro-N-methylbenzamide;
N-(8,9-Difluoro-6-oxo-1,4,5,6-tetrahydro-2H-pyrano[3,4-c]isoquinolin-1-yl)-4-(difluoromethyl)-3-fluoro-N-methylbenzamide;
N-(8,9-Difluoro-6-oxo-1,4,5,6-tetrahydro-2H-pyrano[3,4-c]isoquinolin-1-yl)-4-(difluoromethyl)-N-methylbenzamide;
3-Chloro-N-(8,9-difluoro-6-oxo-1,4,5,6-tetrahydro-2H-pyrano[3,4-c]isoquinolin-1-yl)-4-fluoro-N-methylbenzamide;
N-(8,9-Difluoro-6-oxo-1,2,3,4,5,6-hexahydrobenzo[c][1,7]naphthyridin-1-yl)-7-fluoro-N-methyl-1H-indole-2-carboxamide;
N-(8,9-Difluoro-6-oxo-1,2,3,4,5,6-hexahydrobenzo[c][1,7]naphthyridin-1-yl)-5,6-difluoro-N-methyl-1H-indole-2-carboxamide;
N-(8,9-Difluoro-6-oxo-1,2,3,4,5,6-hexahydrobenzo[c][1,7]naphthyridin-1-yl)-4,6-difluoro-N-methyl-1H-indole-2-carboxamide;
N-(8,9-Difluoro-6-oxo-1,2,3,4,5,6-hexahydrobenzo[c][1,7]naphthyridin-1-yl)-4,5-difluoro-N-methyl-1H-indole-2-carboxamide;
6-Chloro-N-(8,9-difluoro-6-oxo-1,2,3,4,5,6-hexahydrobenzo[c][1,7]naphthyridin-1-yl)-N-methylindolizine-2-carboxamide;
N-(8,9-Difluoro-6-oxo-1,4,5,6-tetrahydro-2H-pyrano[3,4-c]isoquinolin-1-yl)-N-methylbenzo[d]thiazole-2-carboxamide;
N-(8,9-Difluoro-6-oxo-1,4,5,6-tetrahydro-2H-pyrano[3,4-c]isoquinolin-1-yl)-N-methylbenzo[d]oxazole-2-carboxamide;
N-(8,9-Difluoro-6-oxo-1,4,5,6-tetrahydro-2H-pyrano[3,4-c]isoquinolin-1-yl)-N-methyl-3-(trifluoromethyl)-1H-pyrazole-5-carboxamide;
N-(8,9-Difluoro-6-oxo-1,4,5,6-tetrahydro-2H-pyrano[3,4-c]isoquinolin-1-yl)-3-(4-fluorophenyl)-N-methyl-1H-pyrazole-5-carboxamide;
N-(8,9-Difluoro-6-oxo-1,4,5,6-tetrahydro-2H-pyrano[3,4-c]isoquinolin-1-yl)-3-fluoro-N-methylbenzamide;
3-Chloro-N-(8,9-difluoro-6-oxo-1,4,5,6-tetrahydro-2H-pyrano[3,4-c]isoquinolin-1-yl)-N-methylbenzamide;
3-Bromo-N-(8,9-difluoro-6-oxo-1,4,5,6-tetrahydro-2H-pyrano[3,4-c]isoquinolin-1-yl)-N-methylbenzamide;
3-Bromo-N-(8,9-difluoro-6-oxo-1,4,5,6-tetrahydro-2H-pyrano[3,4-c]isoquinolin-1-yl)-4-fluoro-N-methylbenzamide;
4-Bromo-N-(8,9-difluoro-6-oxo-1,4,5,6-tetrahydro-2H-pyrano[3,4-c]isoquinolin-1-yl)-N-methylbenzamide;
3-Fluoro-N-methyl-N-(6-oxo-1,4,5,6-tetrahydro-2H-pyrano[3,4-c]isoquinolin-1-yl)-4-(trifluoromethyl)benzamide;
3-Fluoro-N-methyl-N-(4-oxo-4,5,8,9-tetrahydro-6H-pyrano[3,4-b]thieno[3,4-d]pyridin-9-yl)-4-(trifluoromethyl)benzamide;
N-(8-Cyano-6-oxo-1,4,5,6-tetrahydro-2H-pyrano[3,4-c]isoquinolin-1-yl)-3-fluoro-N-methyl-4-(trifluoromethyl)benzamide;
3-Cyano-N-(8,9-difluoro-6-oxo-1,4,5,6-tetrahydro-2H-pyrano[3,4-c]isoquinolin-1-yl)-N-methylbenzamide;
N-(8,9-Difluoro-6-oxo-1,4,5,6-tetrahydro-2H-pyrano[3,4-c]isoquinolin-1-yl)-3-fluoro-N-methyl-4-(trifluoromethoxy)benzamide;
N-(8,9-Difluoro-6-oxo-1,4,5,6-tetrahydro-2H-pyrano[3,4-c]isoquinolin-1-yl)-3-fluoro-N,4-dimethylbenzamide;
2-(3-Chlorophenyl)-N-(8,9-difluoro-6-oxo-1,4,5,6-tetrahydro-2H-pyrano[3,4-c]isoquinolin-1-yl)-2-hydroxy-N-methylacetamide;
N-(8,9-Difluoro-6-oxo-1,4,5,6-tetrahydro-2H-pyrano[3,4-c]isoquinolin-1-yl)-3,5-difluoro-N-methyl-4-(trifluoromethyl)benzamide;
5-Chloro-N-(8,9-difluoro-6-oxo-1,4,5,6-tetrahydro-2H-pyrano[3,4-c]isoquinolin-1-yl)-N-methylthiophene-3-carboxamide;
3-Chloro-N-(8,9-difluoro-6-oxo-1,4,5,6-tetrahydro-2H-pyrano[3,4-c]isoquinolin-1-yl)-4-fluoro-N-methylbenzenesulfonamide;
3-Chloro-N-(8,9-difluoro-6-oxo-1,4,5,6-tetrahydro-2H-pyrano[3,4-c]isoquinolin-1-yl)-N-methylbenzenesulfonamide;
3-Cyano-N-(8,9-difluoro-6-oxo-1,4,5,6-tetrahydro-2H-pyrano[3,4-c]isoquinolin-1-yl)-N-methylbenzenesulfonamide;
3-Cyano-N-(8,9-difluoro-6-oxo-1,4,5,6-tetrahydro-2H-pyrano[3,4-c]isoquinolin-1-yl)-4-fluoro-N-methylbenzenesulfonamide;
N-(8,9-Difluoro-6-oxo-1,4,5,6-tetrahydro-2H-pyrano[3,4-c]isoquinolin-1-yl)-4-ethyl-3-fluoro-N-methylbenzamide;
N-(8,9-difluoro-6-oxo-1,4,5,6-tetrahydro-2H-pyrano[3,4-c]isoquinolin-1-yl)indolizine-2-carboxamide;
N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
N-(8,9-difluoro-3,3-dioxido-6-oxo-1,4,5,6-tetrahydro-2H-thiopyrano[3,4-c]isoquinolin-1-yl)-8-fluoro-N-methylindolizine-2-carboxamide;
N-(8,9-difluoro-6-oxo-1,4,5,6-tetrahydro-2H-pyrano[3,4-c]isoquinolin-1-yl)-4-(difluoromethoxy)-3-fluoro-N-methylbenzamide;
N-(8,9-difluoro-6-oxo-1,4,5,6-tetrahydro-2H-pyrano[3,4-c]isoquinolin-1-yl)-4-(2-hydroxypropan-2-yl)-N-methylbenzamide;
N-(8,9-difluoro-6-oxo-1,4,5,6-tetrahydro-2H-pyrano[3,4-c]isoquinolin-1-yl)-3-(2-hydroxypropan-2-yl)-N-methylbenzamide;
N-(8,9-difluoro-6-oxo-1,4,5,6-tetrahydro-2H-pyrano[3,4-c]isoquinolin-1-yl)-5,6-difluoro-1H-indole-2-carboxamide;
N-(8,9-difluoro-6-oxo-1,4,5,6-tetrahydro-2H-pyrano[3,4-c]isoquinolin-1-yl)-4,6-difluoro-1H-indole-2-carboxamide;
N-(8,9-difluoro-6-oxo-1,4,5,6-tetrahydro-2H-pyrano[3,4-c]isoquinolin-1-yl)-3-fluoro-4-(trifluoromethyl)benzamide;
4-chloro-N-(8,9-difluoro-6-oxo-1,4,5,6-tetrahydro-2H-pyrano[3,4-c]isoquinolin-1-yl)-3-fluorobenzamide;
N-(8,9-difluoro-6-oxo-1,2,3,4,5,6-hexahydrobenzo[c][1,7]naphthyridin-1-yl)-8-fluoro-N-methylindolizine-2-carboxamide;
N-(8,9-difluoro-6-oxo-1,2,3,4,5,6-hexahydrobenzo[c][1,7]naphthyridin-1-yl)-7-fluoro-N-methylindolizine-2-carboxamide;
4-bromo-N-(8,9-difluoro-6-oxo-1,2,3,4,5,6-hexahydrobenzo[c][1,7]naphthyridin-1-yl)-3,5-difluoro-N-methylbenzamide;
4-bromo-3,5-difluoro-N-(8-fluoro-6-oxo-1,4,5,6-tetrahydro-2H-pyrano[3,4-c]isoquinolin-1-yl)-N-methylbenzamide;
4-chloro-N-(8,9-difluoro-6-oxo-1,2,3,4,5,6-hexahydrobenzo[c][1,7]naphthyridin-1-yl)-3,5-difluoro-N-methylbenzamide;
4-bromo-3,5-difluoro-N-(8-fluoro-6-oxo-1,2,3,4,5,6-hexahydrobenzo[c][1,7]naphthyridin-1-yl)-N-methylbenzamide;
4-bromo-N-(8,9-difluoro-6-oxo-1,4,5,6-tetrahydro-2H-pyrano[3,4-c]isoquinolin-1-yl)-3,5-difluoro-N-methylbenzamide;
4-chloro-N-(8,9-difluoro-6-oxo-1,4,5,6-tetrahydro-2H-pyrano[3,4-c]isoquinolin-1-yl)-3,5-difluoro-N-methylbenzamide;
4-chloro-3,5-difluoro-N-(8-fluoro-6-oxo-1,4,5,6-tetrahydro-2H-pyrano[3,4-c]isoquinolin-1-yl)-N-methylbenzamide;
4-chloro-3,5-difluoro-N-(8-fluoro-6-oxo-1,2,3,4,5,6-hexahydrobenzo[c][1,7]naphthyridin-1-yl)-N-methylbenzamide;
4-(difluoromethyl)-3,5-difluoro-N-(8-fluoro-6-oxo-1,4,5,6-tetrahydro-2H-pyrano[3,4-c]isoquinolin-1-yl)-N-methylbenzamide;
N-(8,9-difluoro-6-oxo-1,4,5,6-tetrahydro-2H-pyrano[3,4-c]isoquinolin-1-yl)-4-(difluoromethyl)-3,5-difluoro-N-methylbenzamide;
4-(difluoromethyl)-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,2,3,4,5,6-hexahydrobenzo[c][1,7]naphthyridin-1-yl)-4-(difluoromethyl)-3,5-difluoro-N-methylbenzamide;
4-(difluoromethyl)-3,5-difluoro-N-(8-fluoro-6-oxo-1,2,3,4,5,6-hexahydrobenzo[c][1,7]naphthyridin-1-yl)-N-methylbenzamide;
N-(8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-N,1-dimethyl-1H-pyrazole-4-carboxamide;
N-(8,9-difluoro-6-oxo-1,4,5,6-tetrahydro-2H-pyrano[3,4-c]isoquinolin-1-yl)-N-methyl-2-phenylacrylamide;
N-(8,9-difluoro-6-oxo-1,4,5,6-tetrahydro-2H-pyrano[3,4-c]isoquinolin-1-yl)-5-fluoro-N-methyl-1H-pyrrolo[2,3-b]pyridine-2-carboxamide;
5-chloro-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
5-bromo-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-3-carboxamide;
5-bromo-N-(8,9-difluoro-6-oxo-1,4,5,6-tetrahydro-2H-pyrano[3,4-c]isoquinolin-1-yl)-N-methylthiophene-3-carboxamide;
N-(8,9-difluoro-6-oxo-1,4,5,6-tetrahydro-2H-pyrano[3,4-c]isoquinolin-1-yl)-N-methyl-2,3-dihydro-1H-indene-2-carboxamide;
1-(tert-butyl)-N-(8,9-difluoro-6-oxo-1,4,5,6-tetrahydro-2H-pyrano[3,4-c]isoquinolin-1-yl)-N-methyl-1H-pyrazole-4-carboxamide;
N-(8,9-difluoro-6-oxo-1,4,5,6-tetrahydro-2H-pyrano[3,4-c]isoquinolin-1-yl)-N-methyl-1-(trifluoromethyl)-1H-pyrazole-4-carboxamide;
4-bromo-N-(8,9-difluoro-6-oxo-1,4,5,6-tetrahydro-2H-pyrano[3,4-c]isoquinolin-1-yl)-N-methyl-1H-pyrrole-2-carboxamide;
2-amino-2-(4-chlorophenyl)-N-(8,9-difluoro-6-oxo-1,4,5,6-tetrahydro-2H-pyrano[3,4-c]isoquinolin-1-yl)-N-methylacetamide;
5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
2-amino-2-(3-chlorophenyl)-N-(8,9-difluoro-6-oxo-1,4,5,6-tetrahydro-2H-pyrano[3,4-c]isoquinolin-1-yl)-N-methylacetamide;
5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
5-chloro-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
N-(8,9-difluoro-6-oxo-1,2,3,4,5,6-hexahydrobenzo[c][1,7]naphthyridin-1-yl)-4-(difluoromethyl)-N-methyl-1H-indole-2-carboxamide;
4-(difluoromethyl)-N-(8-fluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-1-hydroxy-N-methylcyclohexane-1-carboxamide;
N-(8,9-difluoro-6-oxo-1,4,5,6-tetrahydro-2H-pyrano[3,4-c]isoquinolin-1-yl)-6-(difluoromethyl)-5-fluoro-N-methyl-1H-indole-2-carboxamide;
5-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-b]pyridine-2-carboxamide;
5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
6-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methyl-5-(trifluoromethyl)-1H-pyrrolo[3,2-b]pyridine-2-carboxamide;
N-(8,9-difluoro-6-oxo-1,2,3,4,5,6-hexahydrobenzo[c][1,7]naphthyridin-1-yl)-6-(difluoromethyl)-5-fluoro-N-methyl-1H-indole-2-carboxamide;
N-(8,9-difluoro-6-oxo-1,2,3,4,5,6-hexahydrobenzo[c][1,7]naphthyridin-1-yl)-6-fluoro-N-methylindolizine-2-carboxamide;
N-(8,9-difluoro-6-oxo-1,4,5,6-tetrahydro-2H-pyrano[3,4-c]isoquinolin-1-yl)-6-fluoro-N-methylindolizine-2-carboxamide;
N-(8,9-difluoro-6-oxo-1,4,5,6-tetrahydro-2H-pyrano[3,4-c]isoquinolin-1-yl)-N-methyl-2-(trifluoromethyl)-1H-imidazole-5-carboxamide;
N-(8,9-difluoro-6-oxo-1,4,5,6-tetrahydro-2H-pyrano[3,4-c]isoquinolin-1-yl)-5-fluoro-N-methyl-6-oxo-1,6-dihydropyridine-3-carboxamide;
N-(8,9-difluoro-6-oxo-1,4,5,6-tetrahydro-2H-pyrano[3,4-c]isoquinolin-1-yl)-N-methylindolizine-6-carboxamide;
N-(8,9-difluoro-6-oxo-1,4,5,6-tetrahydro-2H-pyrano[3,4-c]isoquinolin-1-yl)-N-methylindolizine-7-carboxamide;
N-(8,9-difluoro-6-oxo-1,4,5,6-tetrahydro-2H-pyrano[3,4-c]isoquinolin-1-yl)-2-hydroxy-N-methyl-2,2-diphenylacetamide;
N-(8,9-difluoro-6-oxo-1,4,5,6-tetrahydro-2H-pyrano[3,4-c]isoquinolin-1-yl)-4-(difluoromethyl)-6-fluoro-N-methyl-1H-indole-2-carboxamide;
N-(8,9-difluoro-6-oxo-1,4,5,6-tetrahydro-2H-pyrano[3,4-c]isoquinolin-1-yl)-7-fluoro-N-methylindolizine-2-carboxamide;
N-(8,9-difluoro-6-oxo-1,4,5,6-tetrahydro-2H-pyrano[3,4-c]isoquinolin-1-yl)-5-fluoro-N-methyl-1H-pyrrolo[3,2-b]pyridine-2-carboxamide;
N-(8,9-difluoro-6-oxo-1,4,5,6-tetrahydro-2H-pyrano[3,4-c]isoquinolin-1-yl)-5,6-difluoro-N-methyl-2,3-dihydro-1H-indene-2-carboxamide;
N-(8,9-difluoro-6-oxo-1,4,5,6-tetrahydro-2H-pyrano[3,4-c]isoquinolin-1-yl)-1-(4-fluorophenyl)-N-methyl-1H-pyrazole-4-carboxamide;
N-(8,9-difluoro-6-oxo-1,4,5,6-tetrahydro-2H-pyrano[3,4-c]isoquinolin-1-yl)-3-(4-fluorophenyl)-N-methylisoxazole-5-carboxamide;
N-(8,9-difluoro-6-oxo-1,4,5,6-tetrahydro-2H-pyrano[3,4-c]isoquinolin-1-yl)-4-(3-fluorophenoxy)-N-methylbenzamide;
4-(difluoromethyl)-6-fluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-5-methyl-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-6-methoxy-1,4-dihydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-N-methyl-5,6,7,8-tetrahydroindolizine-2-carboxamide;
4-bromo-N-(8,9-difluoro-6-oxo-1,4,5,6-tetrahydro-2H-pyrano[3,4-c]isoquinolin-1-yl)-2,3-difluoro-N-methylbenzamide;
4-bromo-N-(8,9-difluoro-6-oxo-1,4,5,6-tetrahydro-2H-pyrano[3,4-c]isoquinolin-1-yl)-2,5-difluoro-N-methylbenzamide;
4-bromo-N-(8,9-difluoro-6-oxo-1,4,5,6-tetrahydro-2H-pyrano[3,4-c]isoquinolin-1-yl)-2,6-difluoro-N-methylbenzamide;
N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-2-carboxamide;
N-(8,9-difluoro-6-oxo-1,4,5,6-tetrahydro-2H-pyrano[3,4-c]isoquinolin-1-yl)-N-methylindolizine-3-carboxamide;
N-(8,9-difluoro-6-oxo-1,4,5,6-tetrahydro-2H-pyrano[3,4-c]isoquinolin-1-yl)-4-(4-fluorophenoxy)-N-methylbenzamide;
N-(8,9-difluoro-6-oxo-1,4,5,6-tetrahydro-2H-pyrano[3,4-c]isoquinolin-1-yl)-4′-fluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-c]pyridine-2-carboxamide;
2-(3-bromophenyl)-N-(8,9-difluoro-6-oxo-1,4,5,6-tetrahydro-2H-pyrano[3,4-c]isoquinolin-1-yl)-2,2-difluoro-N-methylacetamide;
2-(4-bromophenyl)-N-(8,9-difluoro-6-oxo-1,4,5,6-tetrahydro-2H-pyrano[3,4-c]isoquinolin-1-yl)-2,2-difluoro-N-methylacetamide;
N-(8,9-difluoro-6-oxo-1,4,5,6-tetrahydro-2H-pyrano[3,4-c]isoquinolin-1-yl)-4-(difluoromethyl)-5-fluoro-N-methyl-1H-indole-2-carboxamide;
4-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,2,3,4,5,6-hexahydrobenzo[c][1,7]naphthyridin-1-yl)-4-(difluoromethyl)-6-fluoro-N-methyl-1H-indole-2-carboxamide;
4-(difluoromethyl)-6-fluoro-N-(8-fluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methylindolizine-1-carboxamide;
N-(8,9-difluoro-6-oxo-1,4,5,6-tetrahydro-2H-pyrano[3,4-c]isoquinolin-1-yl)-2,4′-difluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-5-(4-fluorophenyl)-N-methylisoxazole-3-carboxamide;
N-(8,9-difluoro-6-oxo-1,2,3,4,5,6-hexahydrobenzo[c][1,7]naphthyridin-1-yl)-4-(difluoromethyl)-5-fluoro-N-methyl-1H-indole-2-carboxamide;
N-(8,9-difluoro-6-oxo-1,4,5,6-tetrahydro-2H-pyrano[3,4-c]isoquinolin-1-yl)-5-(difluoromethyl)-N-methylindolizine-2-carboxamide;
5-bromo-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
N-(8,9-difluoro-6-oxo-1,4,5,6-tetrahydro-2H-pyrano[3,4-c]isoquinolin-1-yl)-N-methylbicyclo[4.2.0]octa-1(6),2,4-triene-7-carboxamide;
4-(difluoromethyl)-5-fluoro-N-(8-fluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-2-hydroxy-N-methyl-2,3-dihydro-1H-indene-2-carboxamide;
N-(8,9-difluoro-6-oxo-1,4,5,6-tetrahydro-2H-pyrano[3,4-c]isoquinolin-1-yl)-5,6-difluoro-2-hydroxy-N-methyl-2,3-dihydro-1H-indene-2-carboxamide;
N-(8,9-difluoro-6-oxo-1,4,5,6-tetrahydro-2H-pyrano[3,4-c]isoquinolin-1-yl)-N,2-dimethylindolizine-6-carboxamide;
N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
N-(8,9-difluoro-6-oxo-1,4,5,6-tetrahydro-2H-pyrano[3,4-c]isoquinolin-1-yl)-4-fluoro-N-methylbicyclo[4.2.0]octa-1(6),2,4-triene-7-carboxamide;
N-(6-(2-aminoethoxy)-8,9-difluoro-1,4-dihydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-N-methyl-1-(thiophen-3-yl)azetidine-3-carboxamide;
N-(8,9-difluoro-6-oxo-1,4,5,6-tetrahydro-2H-pyrano[3,4-c]isoquinolin-1-yl)-3′-fluoro-N-methyl-[1,1′-biphenyl]-4-carboxamide;
1-(4-bromothiophen-3-yl)-N-(8,9-difluoro-6-oxo-1,4,5,6-tetrahydro-2H-pyrano[3,4-c]isoquinolin-1-yl)-N-methylazetidine-3-carboxamide;
N-(8,9-difluoro-6-oxo-1,4,5,6-tetrahydro-2H-pyrano[3,4-c]isoquinolin-1-yl)-2′-fluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-3′,5′-difluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-2,3′,5′-trifluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-N-methyl-[1,1′-biphenyl]-3-carboxamide;
N-(8,9-difluoro-6-oxo-1,4,5,6-tetrahydro-2H-pyrano[3,4-c]isoquinolin-1-yl)-4′-fluoro-N-methyl-[1,1′-biphenyl]-3-carboxamide;
N-(8,9-difluoro-6-oxo-1,4,5,6-tetrahydro-2H-pyrano[3,4-c]isoquinolin-1-yl)-3′-fluoro-N-methyl-[1,1′-biphenyl]-3-carboxamide;
N-(8,9-difluoro-6-oxo-1,4,5,6-tetrahydro-2H-pyrano[3,4-c]isoquinolin-1-yl)-3′,5-difluoro-N-methyl-[1,1′-biphenyl]-3-carboxamide;
N-(8,9-difluoro-6-oxo-1,4,5,6-tetrahydro-2H-pyrano[3,4-c]isoquinolin-1-yl)-N-methyl-3-phenoxybenzamide;
N-(8,9-difluoro-6-oxo-1,4,5,6-tetrahydro-2H-pyrano[3,4-c]isoquinolin-1-yl)-3-(4-fluorophenoxy)-N-methylbenzamide;
N-(8,9-difluoro-6-oxo-1,4,5,6-tetrahydro-2H-pyrano[3,4-c]isoquinolin-1-yl)-N-methyl-6H-thieno[2,3-b]pyrrole-5-carboxamide;
N-(8,9-difluoro-6-oxo-1,4,5,6-tetrahydro-2H-pyrano[3,4-c]isoquinolin-1-yl)-N-methyl-4-(methylsulfonyl)benzamide;
N-(8,9-difluoro-6-oxo-1,4,5,6-tetrahydro-2H-pyrano[3,4-c]isoquinolin-1-yl)-N-methyl-3-(N-methylsulfamoyl)benzamide;
N-(8,9-difluoro-6-oxo-1,4,5,6-tetrahydro-2H-pyrano[3,4-c]isoquinolin-1-yl)-N-methyl-3-(methylsulfonamido)benzamide;
N-(8,9-difluoro-6-oxo-1,4,5,6-tetrahydro-2H-pyrano[3,4-c]isoquinolin-1-yl)-N-methyl-3-(methylsulfonyl)benzamide;
N-(8,9-difluoro-6-oxo-1,4,5,6-tetrahydro-2H-pyrano[3,4-c]isoquinolin-1-yl)-N-methylimidazo[1,5-a]pyridine-6-carboxamide;
1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
N-(8,9-difluoro-6-oxo-1,2,3,4,5,6-hexahydrobenzo[c][1,7]naphthyridin-1-yl)-3′-fluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-3-fluoro-N-methyl-5-phenoxybenzamide;
N-(8,9-difluoro-6-oxo-1,2,3,4,5,6-hexahydrobenzo[c][1,7]naphthyridin-1-yl)-3′-fluoro-N-methyl-[1,1′-biphenyl]-3-carboxamide;
N-(8,9-difluoro-6-oxo-1,4,5,6-tetrahydro-2H-pyrano[3,4-c]isoquinolin-1-yl)-N-methylimidazo[1,2-a]pyridine-6-carboxamide;
N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
6-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
N-(8,9-difluoro-6-oxo-1,4,5,6-tetrahydro-2H-pyrano[3,4-c]isoquinolin-1-yl)-5-fluoro-N-methyl-1H-pyrrolo[2,3-c]pyridine-2-carboxamide;
N-(8,9-difluoro-6-oxo-1,4,5,6-tetrahydro-2H-pyrano[3,4-c]isoquinolin-1-yl)-N-methyl-2-(trifluoromethyl)indolizine-6-carboxamide;
4-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
N-(8,9-difluoro-6-oxo-1,4,5,6-tetrahydro-2H-pyrano[3,4-c]isoquinolin-1-yl)-2-(difluoromethyl)-N-methylisonicotinamide;
N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
N-(8,9-difluoro-5-(2-hydroxyethyl)-6-oxo-1,4,5,6-tetrahydro-2H-pyrano[3,4-c]isoquinolin-1-yl)-5,6-difluoro-N-methyl-1H-indole-2-carboxamide;
N-(5-(2-aminoethyl)-8,9-difluoro-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-6-(2-hydroxyethoxy)-1,4-dihydro-2H-pyrano[3,4-c]isoquinolin-1-yl)-5,6-difluoro-N-methyl-1H-indole-2-carboxamide;
N-(8,9-difluoro-6-oxo-1,2,3,4,5,6-hexahydrobenzo[c][1,7]naphthyridin-1-yl)-N-methyl-3-(N-methylsulfamoyl)benzamide;
N-(8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-4,6-dioxo-1,4,5,6-tetrahydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-3-(3-fluorophenoxy)-N-methylbenzamide;
N-(8,9-difluoro-6-oxo-1,4,5,6-tetrahydro-2H-pyrano[3,4-c]isoquinolin-1-yl)-5-methoxy-N-methyl-1H-indole-2-carboxamide;
5-chloro-N-(8,9-difluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N,5-dimethyl-1H-indole-2-carboxamide;
N-(8,9-difluoro-6-oxo-1,4,5,6-tetrahydro-2H-pyrano[3,4-c]isoquinolin-1-yl)-N-methyl-5-(trifluoromethoxy)-1H-indole-2-carboxamide;
N-(8,9-difluoro-6-oxo-1,4,5,6-tetrahydro-2H-pyrano[3,4-c]isoquinolin-1-yl)-5-ethyl-N-methyl-1H-indole-2-carboxamide;
N-(8,9-difluoro-6-oxo-1,4,5,6-tetrahydro-2H-pyrano[3,4-c]isoquinolin-1-yl)-4-methoxy-N-methyl-1H-indole-2-carboxamide;
N-(8,9-difluoro-6-oxo-1,4,5,6-tetrahydro-2H-pyrano[3,4-c]isoquinolin-1-yl)-N,4-dimethyl-1H-indole-2-carboxamide;
1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
2-chloro-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
N-(8,9-difluoro-6-(methylamino)-1,4-dihydro-2H-pyrano[3,4-c]isoquinolin-1-yl)-5,6-difluoro-N-methyl-1H-indole-2-carboxamide;
N-(8,9-difluoro-6-((2-hydroxyethyl)amino)-1,4-dihydro-2H-pyrano[3,4-c]isoquinolin-1-yl)-5,6-difluoro-N-methyl-1H-indole-2-carboxamide;
N-(8,9-difluoro-6-oxo-1,2,3,4,5,6-hexahydrobenzo[c][1,7]naphthyridin-1-yl)-3-(3-fluorophenoxy)-N-methylbenzamide;
4-cyano-N-(8,9-difluoro-6-oxo-1,4,5,6-tetrahydro-2H-pyrano[3,4-c]isoquinolin-1-yl)-6-fluoro-N-methyl-1H-indole-2-carboxamide;
N-(8,9-difluoro-6-oxo-1,4,5,6-tetrahydro-2H-pyrano[3,4-c]isoquinolin-1-yl)-4-ethyl-6-fluoro-N-methyl-1H-indole-2-carboxamide;
4-chloro-N-(8,9-difluoro-6-oxo-1,4,5,6-tetrahydro-2H-pyrano[3,4-c]isoquinolin-1-yl)-6-fluoro-N-methyl-1H-indole-2-carboxamide;
N-(8,9-difluoro-6-oxo-1,4,5,6-tetrahydro-2H-pyrano[3,4-c]isoquinolin-1-yl)-6-fluoro-N,4-dimethyl-1H-indole-2-carboxamide;
5-cyano-N-(8,9-difluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methyl-5-(methylsulfonyl)-1H-indole-2-carboxamide;
4-chloro-N-(8,9-difluoro-6-oxo-1,4,5,6-tetrahydro-2H-pyrano[3,4-c]isoquinolin-1-yl)-N-methyl-1H-indole-2-carboxamide;
6-bromo-N-(8,9-difluoro-6-oxo-1,4,5,6-tetrahydro-2H-pyrano[3,4-c]isoquinolin-1-yl)-N-methylindolizine-2-carboxamide;
2-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
3-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
N-(6-((2-aminoethyl)amino)-8,9-difluoro-1,4-dihydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-6-fluoro-4-(1-hydroxyethyl)-N-methyl-1H-indole-2-carboxamide;
N-(8,9-difluoro-6-oxo-1,4,5,6-tetrahydro-2H-pyrano[3,4-c]isoquinolin-1-yl)-6-fluoro-4-(hydroxymethyl)-N-methyl-1H-indole-2-carboxamide;
7-bromo-N-(8,9-difluoro-6-oxo-1,4,5,6-tetrahydro-2H-pyrano[3,4-c]isoquinolin-1-yl)-N-methylfuro[3,2-c]pyridine-2-carboxamide;
3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-6-carboxamide;
3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-6-carboxamide;
3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-5-carboxamide; 3,4-dichloro-N-(8,9-difluoro-6-oxo-1,4,5,6-tetrahydro-2H-pyrano[3,4-c]isoquinolin-1-yl)-N-methylbenzamide;
N-(8,9-difluoro-6-oxo-1,4,5,6-tetrahydro-2H-pyrano[3,4-c]isoquinolin-1-yl)-6-fluoro-N-methyl-4-(methylsulfonamido)-1H-indole-2-carboxamide;
N-(8,9-difluoro-6-oxo-1,4,5,6-tetrahydro-2H-pyrano[3,4-c]isoquinolin-1-yl)-N-methyl-4-(trifluoromethoxy)-1H-indole-2-carboxamide;
N-(8,9-difluoro-6-oxo-1,4,5,6-tetrahydro-2H-pyrano[3,4-c]isoquinolin-1-yl)-6-methoxy-N-methyl-1H-indole-2-carboxamide;
5-bromo-N-(8,9-difluoro-6-oxo-1,4,5,6-tetrahydro-2H-pyrano[3,4-c]isoquinolin-1-yl)-N-methyl-6-(trifluoromethyl)nicotinamide;
3-chloro-N-(8,9-difluoro-6-oxo-1,4,5,6-tetrahydro-2H-pyrano[3,4-c]isoquinolin-1-yl)-N-methylindolizine-6-carboxamide;
N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-6-carboxamide;
N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-5-carboxamide;
N-(8,9-difluoro-6-oxo-1,4,5,6-tetrahydro-2H-pyrano[3,4-c]isoquinolin-1-yl)-3-methoxy-N-methyl-1H-indazole-5-carboxamide;
1-(5-chlorothiophen-3-yl)-N-(8,9-difluoro-6-oxo-1,4,5,6-tetrahydro-2H-pyrano[3,4-c]isoquinolin-1-yl)-N-methylazetidine-3-carboxamide;
7-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
4-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
3-chloro-N-(8,9-difluoro-6-oxo-1,4,5,6-tetrahydro-2H-pyrano[3,4-c]isoquinolin-1-yl)-N-methylindolizine-7-carboxamide;
N-(8,9-difluoro-6-oxo-1,4,5,6-tetrahydro-2H-pyrano[3,4-c]isoquinolin-1-yl)-7-(difluoromethyl)-N-methylindolizine-2-carboxamide;
N-(8,9-difluoro-6-oxo-1,4,5,6-tetrahydro-2H-pyrano[3,4-c]isoquinolin-1-yl)-6-(difluoromethyl)-N-methylindolizine-2-carboxamide;
di-tert-butyl ((2-((8,9-difluoro-6-oxo-1,4,5,6-tetrahydro-2H-pyrano[3,4-c]isoquinolin-1-yl)(methyl)carbamoyl)-5,6-difluoro-1H-indol-1-yl)methyl)phosphate;
5-chloro-N-(8,9-difluoro-6-oxo-1,4,5,6-tetrahydro-2H-pyrano[3,4-c]isoquinolin-1-yl)-N-methyl-6-(trifluoromethyl)nicotinamide;
5-chloro-N-(8-fluoro-6-oxo-1,4,5,6-tetrahydro-2H-pyrano[3,4-c]isoquinolin-1-yl)-N-methyl-6-(trifluoromethyl)nicotinamide;
N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
4-cyano-6-fluoro-N-(8-fluoro-6-oxo-1,4,5,6-tetrahydro-2H-pyrano[3,4-c]isoquinolin-1-yl)-N-methyl-1H-indole-2-carboxamide;
4-bromo-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
N-(8,9-difluoro-6-oxo-1,2,3,4,5,6-hexahydrobenzo[c][1,7]naphthyridin-1-yl)-7-(difluoromethyl)-N-methylindolizine-2-carboxamide;
N-(8,9-difluoro-6-oxo-1,2,3,4,5,6-hexahydrobenzo[c][1,7]naphthyridin-1-yl)-6-(difluoromethyl)-N-methylindolizine-2-carboxamide;
N-(8-fluoro-6-oxo-1,4,5,6-tetrahydro-2H-pyrano[3,4-c]isoquinolin-1-yl)-N-methylindolizine-6-carboxamide;
N-(8,9-difluoro-6-oxo-1,2,3,4,5,6-hexahydrobenzo[c][1,7]naphthyridin-1-yl)-N-methylindolizine-6-carboxamide;
4-ethyl-6-fluoro-N-(8-fluoro-6-oxo-1,4,5,6-tetrahydro-2H-pyrano[3,4-c]isoquinolin-1-yl)-N-methyl-1H-indole-2-carboxamide;
5-cyano-N-(8-fluoro-6-oxo-1,4,5,6-tetrahydro-2H-pyrano[3,4-c]isoquinolin-1-yl)-N-methyl-1H-indole-2-carboxamide;
4-cyano-N-(8,9-difluoro-6-oxo-1,2,3,4,5,6-hexahydrobenzo[c][1,7]naphthyridin-1-yl)-6-fluoro-N-methyl-1H-indole-2-carboxamide;
N-(8,9-difluoro-6-oxo-1,2,3,4,5,6-hexahydrobenzo[c][1,7]naphthyridin-1-yl)-4-ethyl-6-fluoro-N-methyl-1H-indole-2-carboxamide;
N-(8,9-difluoro-6-oxo-1,2,3,4,5,6-hexahydrobenzo[c][1,7]naphthyridin-1-yl)-N-methyl-5-(methylsulfonyl)-1H-indole-2-carboxamide;
5-cyano-N-(8,9-difluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-8-(difluoromethyl)-N-methylindolizine-2-carboxamide;
N-(8,9-difluoro-6-oxo-1,4,5,6-tetrahydro-2H-pyrano[3,4-c]isoquinolin-1-yl)-N-methyl-2,3-dihydro-1H-indene-5-carboxamide;
N-(8,9-difluoro-6-oxo-1,4,5,6-tetrahydro-2H-pyrano[3,4-c]isoquinolin-1-yl)-N-methylbenzo[d]oxazole-6-carboxamide;
4,6-dichloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
5,6-difluoro-N-methyl-N-(4,8,9-trifluoro-6-oxo-1,4,5,6-tetrahydro-2H-pyrano[3,4-c]isoquinolin-1-yl)-1H-indole-2-carboxamide;
N-(8,9-difluoro-6-oxo-1,4,5,6-tetrahydro-2H-pyrano[3,4-c]isoquinolin-1-yl)-5-fluoro-N-methyl-6-(trifluoromethyl)nicotinamide;
5-fluoro-N-(8-fluoro-6-oxo-1,4,5,6-tetrahydro-2H-pyrano[3,4-c]isoquinolin-1-yl)-N-methyl-6-(trifluoromethyl)nicotinamide;
3-chloro-N-(8,9-difluoro-6-oxo-1,4,5,6-tetrahydro-2H-pyrano[3,4-c]isoquinolin-1-yl)-N,1-dimethyl-1H-indazole-5-carboxamide;
3-chloro-N-(8,9-difluoro-6-oxo-1,4,5,6-tetrahydro-2H-pyrano[3,4-c]isoquinolin-1-yl)-1-ethyl-N-methyl-1H-indazole-5-carboxamide;
N-(8,9-difluoro-6-oxo-1,4,5,6-tetrahydro-2H-pyrano[3,4-c]isoquinolin-1-yl)-1-(difluoromethyl)-N,3-dimethyl-1H-indazole-5-carboxamide;
5-chloro-N-(8,9-difluoro-6-oxo-1,2,3,4,5,6-hexahydrobenzo[c][1,7]naphthyridin-1-yl)-N-methyl-6-(trifluoromethyl)nicotinamide;
N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
N-(8-fluoro-6-oxo-1,4,5,6-tetrahydro-2H-pyrano[3,4-c]isoquinolin-1-yl)-N,4-dimethyl-1H-indole-2-carboxamide;
4-chloro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methylbenzo[d]oxazole-5-carboxamide;
N-(8,9-difluoro-6-oxo-1,4,5,6-tetrahydro-2H-pyrano[3,4-c]isoquinolin-1-yl)-N-methylbenzo[d]thiazole-5-carboxamide;
N-(8,9-difluoro-6-oxo-1,4,5,6-tetrahydro-2H-pyrano[3,4-c]isoquinolin-1-yl)-N-methylbenzo[d]thiazole-6-carboxamide;
N-(8,9-difluoro-6-oxo-1,4,5,6-tetrahydro-2H-pyrano[3,4-c]isoquinolin-1-yl)-N-methyl-1H-indazole-5-carboxamide;
N-(8,9-difluoro-6-oxo-1,2,3,4,5,6-hexahydrobenzo[c][1,7]naphthyridin-1-yl)-8-(difluoromethyl)-N-methylindolizine-2-carboxamide;
N-(8,9-difluoro-6-oxo-1,2,3,4,5,6-hexahydrobenzo[c][1,7]naphthyridin-1-yl)-5-(difluoromethyl)-N-methylindolizine-2-carboxamide;
N-(8,9-difluoro-6-oxo-1,2,3,4,5,6-hexahydrobenzo[c][1,7]naphthyridin-1-yl)-5-fluoro-N-methyl-6-(trifluoromethyl)nicotinamide;
N-(8,9-difluoro-6-oxo-1,4,5,6-tetrahydro-2H-pyrano[3,4-c]isoquinolin-1-yl)-3-(difluoromethyl)-N,1-dimethyl-1H-indazole-5-carboxamide;
N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
4-chloro-6-fluoro-N-(8-fluoro-6-oxo-1,4,5,6-tetrahydro-2H-pyrano[3,4-c]isoquinolin-1-yl)-N-methyl-1H-indole-2-carboxamide;
4-chloro-N-(8,9-difluoro-6-oxo-1,2,3,4,5,6-hexahydrobenzo[c][1,7]naphthyridin-1-yl)-N-methyl-1H-indole-2-carboxamide;
N-(8-fluoro-6-oxo-1,4,5,6-tetrahydro-2H-pyrano[3,4-c]isoquinolin-1-yl)-N-methyl-2,3-dihydro-1H-indene-5-carboxamide;
N-(8-fluoro-6-oxo-1,4,5,6-tetrahydro-2H-pyrano[3,4-c]isoquinolin-1-yl)-N-methylbenzo[d]thiazole-5-carboxamide;
N-(8-fluoro-6-oxo-1,4,5,6-tetrahydro-2H-pyrano[3,4-c]isoquinolin-1-yl)-N-methylbenzo[d]thiazole-6-carboxamide;
N-(8-fluoro-6-oxo-1,4,5,6-tetrahydro-2H-pyrano[3,4-c]isoquinolin-1-yl)-N-methyl-1H-indazole-5-carboxamide;
N-(8,9-difluoro-6-oxo-1,4,5,6-tetrahydro-2H-pyrano[3,4-c]isoquinolin-1-yl)-N-methyl-1H-indazole-6-carboxamide;
N-(8,9-difluoro-6-oxo-1,4,5,6-tetrahydro-2H-pyrano[3,4-c]isoquinolin-1-yl)-N-methyl-1H-benzo[d]imidazole-6-carboxamide;
2-chloro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
2-chloro-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
6-fluoro-N-(8-fluoro-6-oxo-1,4,5,6-tetrahydro-2H-pyrano[3,4-c]isoquinolin-1-yl)-N,4-dimethyl-1H-indole-2-carboxamide;
N-(8,9-difluoro-6-oxo-1,2,3,4,5,6-hexahydrobenzo[c][1,7]naphthyridin-1-yl)-N,4-dimethyl-1H-indole-2-carboxamide;
N-(8,9-difluoro-6-oxo-1,2,3,4,5,6-hexahydrobenzo[c][1,7]naphthyridin-1-yl)-6-fluoro-N,4-dimethyl-1H-indole-2-carboxamide;
4-chloro-N-(8,9-difluoro-6-oxo-1,2,3,4,5,6-hexahydrobenzo[c][1,7]naphthyridin-1-yl)-6-fluoro-N-methyl-1H-indole-2-carboxamide;
N-(8,9-difluoro-6-oxo-1,2,3,4,5,6-hexahydrobenzo[c][1,7]naphthyridin-1-yl)-N-methylbenzo[d]thiazole-5-carboxamide;
N-(8,9-difluoro-6-oxo-1,2,3,4,5,6-hexahydrobenzo[c][1,7]naphthyridin-1-yl)-N-methylbenzo[d]thiazole-6-carboxamide;
N-(8,9-difluoro-6-oxo-1,2,3,4,5,6-hexahydrophenanthridin-1-yl)-N-methyl-1H-indole-2-carboxamide;
2-cyano-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
N-(8-fluoro-6-oxo-1,4,5,6-tetrahydro-2H-pyrano[3,4-c]isoquinolin-1-yl)-N-methyl-1H-indazole-6-carboxamide;
N-(8,9-difluoro-6-oxo-1,2,3,4,5,6-hexahydrobenzo[c][1,7]naphthyridin-1-yl)-N-methyl-2,3-dihydro-1H-indene-5-carboxamide;
N-(8,9-difluoro-6-oxo-1,2,3,4,5,6-hexahydrobenzo[c][1,7]naphthyridin-1-yl)-N-methyl-1H-indazole-5-carboxamide;
N-(8-fluoro-6-oxo-1,2,3,4,5,6-hexahydrophenanthridin-1-yl)-N-methyl-1H-indole-2-carboxamide;
2-chloro-N-(8,9-difluoro-4,6-dioxo-1,4,5,6-tetrahydro-2H-pyrano[3,4-c]isoquinolin-1-yl)-N-methyl-4H-thieno[3,2-b]pyrrole-5-carboxamide;
N-(8,9-difluoro-4,6-dioxo-1,4,5,6-tetrahydro-2H-pyrano[3,4-c]isoquinolin-1-yl)-6-(difluoromethyl)-5-fluoro-N-methyl-1H-indole-2-carboxamide;
6-(difluoromethyl)-5-fluoro-N-(8-fluoro-6-oxo-1,2,3,4,5,6-hexahydrophenanthridin-1-yl)-N-methyl-1H-indole-2-carboxamide;
N-(8,9-difluoro-6-oxo-1,2,3,4,5,6-hexahydrophenanthridin-1-yl)-5-fluoro-N-methyl-1H-indole-2-carboxamide;
N-(8,9-difluoro-6-oxo-1,2,3,4,5,6-hexahydrophenanthridin-1-yl)-5,6-difluoro-N-methyl-1H-indole-2-carboxamide;
N-(8,9-difluoro-6-oxo-1,2,3,4,5,6-hexahydrophenanthridin-1-yl)-6-(difluoromethyl)-5-fluoro-N-methyl-1H-indole-2-carboxamide;
N-(8,9-difluoro-6-oxo-1,2,3,4,5,6-hexahydrophenanthridin-1-yl)-4-(difluoromethyl)-6-fluoro-N-methyl-1H-indole-2-carboxamide;
N-(8,9-difluoro-6-oxo-1,2,3,4,5,6-hexahydrophenanthridin-1-yl)-N,4-dimethyl-1H-indole-2-carboxamide;
N-(8,9-difluoro-6-oxo-1,2,3,4,5,6-hexahydrophenanthridin-1-yl)-6-fluoro-N,4-dimethyl-1H-indole-2-carboxamide;
1-((4-bromo-3-fluorobenzyl)(methyl)amino)-8,9-difluoro-1,5-dihydro-2H-pyrano[3,4-c]isoquinolin-6(4H)-one;
2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
5-fluoro-N-(8-fluoro-6-oxo-1,2,3,4,5,6-hexahydrophenanthridin-1-yl)-N-methyl-1H-indole-2-carboxamide;
5,6-difluoro-N-(8-fluoro-6-oxo-1,2,3,4,5,6-hexahydrophenanthridin-1-yl)-N-methyl-1H-indole-2-carboxamide;
N-(8-fluoro-6-oxo-1,2,3,4,5,6-hexahydrophenanthridin-1-yl)-N,4-dimethyl-1H-indole-2-carboxamide;
6-fluoro-N-(8-fluoro-6-oxo-1,2,3,4,5,6-hexahydrophenanthridin-1-yl)-N,4-dimethyl-1H-indole-2-carboxamide;
4-(difluoromethyl)-6-fluoro-N-(8-fluoro-6-oxo-1,2,3,4,5,6-hexahydrophenanthridin-1-yl)-N-methyl-1H-indole-2-carboxamide;
1-(((5,6-difluoro-1H-indol-2-yl)methyl)(methyl)amino)-8,9-difluoro-1,5-dihydro-2H-pyrano[3,4-c]isoquinolin-6(4H)-one;
N-(8,9-difluoro-6-oxo-1,2,3,4,5,6-hexahydrobenzo[c][1,7]naphthyridin-1-yl)-N-methyl-1H-indazole-6-carboxamide;
2-fluoro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
N-(8,9-difluoro-6-oxo-1,2,3,4,5,6-hexahydrobenzo[c][1,7]naphthyridin-1-yl)-2-fluoro-N-methyl-4H-thieno[3,2-b]pyrrole-5-carboxamide;
N-(8,9-difluoro-4-hydroxy-6-oxo-1,2,3,4,5,6-hexahydrophenanthridin-1-yl)-5-fluoro-N-methyl-1H-indole-2-carboxamide;
N-(8,9-difluoro-4-hydroxy-6-oxo-1,2,3,4,5,6-hexahydrophenanthridin-1-yl)-5,6-difluoro-N-methyl-1H-indole-2-carboxamide;
N-(8,9-difluoro-4-hydroxy-6-oxo-1,2,3,4,5,6-hexahydrophenanthridin-1-yl)-N-methyl-1H-indole-2-carboxamide;
N-(8,9-difluoro-6-oxo-1,2,3,4,5,6-hexahydrobenzo[c][1,7]naphthyridin-1-yl)-N-methylindolizine-2-carboxamide;
8-chloro-N-(8,9-difluoro-6-oxo-1,2,3,4,5,6-hexahydrobenzo[c][1,7]naphthyridin-1-yl)-N-methylindolizine-2-carboxamide;
N-(8,9-Difluoro-6-oxo-1,4,5,6-tetrahydro-2H-pyrano[3,4-c]isoquinolin-1-yl)-2,2-difluoro-N-methyl-2-phenylacetamide;
N-(8,9-Difluoro-6-oxo-1,4,5,6-tetrahydro-2H-pyrano[3,4-c]isoquinolin-1-yl-1-d)-5,6-difluoro-N-(methyl-13C-d3)-1H-indole-2-carboxamide;
N-(8,9-Difluoro-6-oxo-1,4,5,6-tetrahydro-2H-pyrano[3,4-c]isoquinolin-1-yl-1-d)-6-(difluoromethyl)-5-fluoro-N-(methyl-13C-d3)-1H-indole-2-carboxamide; and
2-Chloro-N-(8,9-difluoro-6-oxo-1,4,5,6-tetrahydro-2H-pyrano[3,4-c]isoquinolin-1-yl-1-d)-N-(methyl-13C-d3)-4H-thieno[3,2-b]pyrrole-5-carboxamide;
   or a salt, solvate, prodrug, isotopically labelled, stereoisomer, any mixture of stereoisomers, tautomer, and/or any mixture of tautomers thereof.

In certain embodiments, the compound is:
(R)-N-(8-fluoro-6-oxo-1,4,5,6-tetrahydro-2H-pyrano[3,4-c]isoquinolin-1-yl)-N-methyl-1H-indole-2-carboxamide;
(S)-N-(8-fluoro-6-oxo-1,4,5,6-tetrahydro-2H-pyrano[3,4-c]isoquinolin-1-yl)-N-methyl-1H-indole-2-carboxamide;
(2S)-N-(8-fluoro-6-oxo-1,4,5,6-tetrahydro-2H-pyrano[3,4-c]isoquinolin-1-yl)-N-methylindoline-2-carboxamide;
(2S)-N-(8-fluoro-6-oxo-1,4,5,6-tetrahydro-2H-pyrano[3,4-c]isoquinolin-(1R)-yl)-N-methylindoline-2-carboxamide;
(2S)-N-(8-fluoro-6-oxo-1,4,5,6-tetrahydro-2H-pyrano[3,4-c]isoquinolin-(1S)-yl)-N-methylindoline-2-carboxamide;
(2R)-N-(8-fluoro-6-oxo-1,4,5,6-tetrahydro-2H-pyrano[3,4-c]isoquinolin-1-yl)-N-methylindoline-2-carboxamide;
(2R)-N-(8-fluoro-6-oxo-1,4,5,6-tetrahydro-2H-pyrano[3,4-c]isoquinolin-(1R)-yl)-N-methylindoline-2-carboxamide;
(2R)-N-(8-fluoro-6-oxo-1,4,5,6-tetrahydro-2H-pyrano[3,4-c]isoquinolin-(1S)-yl)-N-methylindoline-2-carboxamide;
(N-(8,9-difluoro-6-oxo-1,4,5,6-tetrahydro-2H-pyrano[3,4-c]isoquinolin-(1R)-yl)-5-fluoro-N-methylindoline-(2R)-carboxamide;
N-(8,9-difluoro-6-oxo-1,4,5,6-tetrahydro-2H-pyrano[3,4-c]isoquinolin-(1S)-yl)-5-fluoro-N-methylindoline-(2R)-carboxamide;
N-(8,9-difluoro-6-oxo-1,4,5,6-tetrahydro-2H-pyrano[3,4-c]isoquinolin-(1R)-yl)-5-fluoro-N-methylindoline-(2S)-carboxamide;
N-(8,9-difluoro-6-oxo-1,4,5,6-tetrahydro-2H-pyrano[3,4-c]isoquinolin-(1S)-yl)-5-fluoro-N-methylindoline-(2S)-carboxamide;
(R)-N-(8,9-difluoro-6-oxo-1,4,5,6-tetrahydro-2H-pyrano[3,4-c]isoquinolin-1-yl)-8-fluoro-N-methylindolizine-2-carboxamide;
(S)-N-(8,9-difluoro-6-oxo-1,4,5,6-tetrahydro-2H-pyrano[3,4-c]isoquinolin-1-yl)-8-fluoro-N-methylindolizine-2-carboxamide;
(R)-N-(8,9-difluoro-6-oxo-1,4,5,6-tetrahydro-2H-pyrano[3,4-c]isoquinolin-1-yl)-N-methylindolizine-2-carboxamide;
(S)-N-(8,9-difluoro-6-oxo-1,4,5,6-tetrahydro-2H-pyrano[3,4-c]isoquinolin-1-yl)-N-methylindolizine-2-carboxamide;
N-(8,9-difluoro-6-oxo-1,4,5,6-tetrahydro-2H-pyrano[3,4-c]isoquinolin-(1R)-yl)-4,6-difluoro-N-methylindoline-(2R)-carboxamide;
N-(8,9-difluoro-6-oxo-1,4,5,6-tetrahydro-2H-pyrano[3,4-c]isoquinolin-(1R)-yl)-4,6-difluoro-N-methylindoline-(2S)-carboxamide;
N-(8,9-difluoro-6-oxo-1,4,5,6-tetrahydro-2H-pyrano[3,4-c]isoquinolin-(1S)-yl)-4,6-difluoro-N-methylindoline-(2R)-carboxamide;
N-(8,9-difluoro-6-oxo-1,4,5,6-tetrahydro-2H-pyrano[3,4-c]isoquinolin-(1S)-yl)-4,6-difluoro-N-methylindoline-(2S)-carboxamide;
(S)-N-((S)-8,9-difluoro-6-oxo-1,4,5,6-tetrahydro-2H-pyrano[3,4-c]isoquinolin-1-yl)-4,6-difluoro-N-methylindoline-2-carboxamide;
(S)-N-((R)-8,9-difluoro-6-oxo-1,4,5,6-tetrahydro-2H-pyrano[3,4-c]isoquinolin-1-yl)-4,6-difluoro-N-methylindoline-2-carboxamide;
(R)-N-((S)-8,9-difluoro-6-oxo-1,4,5,6-tetrahydro-2H-pyrano[3,4-c]isoquinolin-1-yl)-4,6-difluoro-N-methylindoline-2-carboxamide;
(S)-N-((R)-8,9-difluoro-6-oxo-1,4,5,6-tetrahydro-2H-pyrano[3,4-c]isoquinolin-1-yl)-4,6-difluoro-N-methylindoline-2-carboxamide;
(R)-8-chloro-N-(8,9-difluoro-6-oxo-1,4,5,6-tetrahydro-2H-pyrano[3,4-c]isoquinolin-1-yl)-N-methylindolizine-2-carboxamide;
(S)-8-chloro-N-(8,9-difluoro-6-oxo-1,4,5,6-tetrahydro-2H-pyrano[3,4-c]isoquinolin-1-yl)-N-methylindolizine-2-carboxamide;
(R)-4-fluoro-N-(8-fluoro-6-oxo-1,4,5,6-tetrahydro-2H-pyrano[3,4-c]isoquinolin-1-yl)-N-methyl-1H-indole-2-carboxamide;
(S)-4-fluoro-N-(8-fluoro-6-oxo-1,4,5,6-tetrahydro-2H-pyrano[3,4-c]isoquinolin-1-yl)-N-methyl-1H-indole-2-carboxamide;
(R)-N-(8,9-difluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-1H-indole-2-carboxamide;
(R)-N-(8,9-difluoro-6-oxo-1,4,5,6-tetrahydro-2H-pyrano[3,4-c]isoquinolin-1-yl)-4-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-4-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-5-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-5-fluoro-N-methyl-1H-indole-2-carboxamide;
(R)-5-fluoro-N-(8-fluoro-6-oxo-1,4,5,6-tetrahydro-2H-pyrano[3,4-c]isoquinolin-1-yl)-N-methyl-1H-indole-2-carboxamide;
(S)-5-fluoro-N-(8-fluoro-6-oxo-1,4,5,6-tetrahydro-2H-pyrano[3,4-c]isoquinolin-1-yl)-N-methyl-1H-indole-2-carboxamide;
(R)-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-7-fluoro-N-(8-fluoro-6-oxo-1,4,5,6-tetrahydro-2H-pyrano[3,4-c]isoquinolin-1-yl)-N-methyl-1H-indole-2-carboxamide;
(S)-7-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4,6-difluoro-N-(8-fluoro-6-oxo-1,4,5,6-tetrahydro-2H-pyrano[3,4-c]isoquinolin-1-yl)-N-methyl-1H-indole-2-carboxamide;
(S)-4,6-difluoro-N-(8-fluoro-6-oxo-1,4,5,6-tetrahydro-2H-pyrano[3,4-c]isoquinolin-1-yl)-N-methyl-1H-indole-2-carboxamide;
(R)-4,5-difluoro-N-(8-fluoro-6-oxo-1,4,5,6-tetrahydro-2H-pyrano[3,4-c]isoquinolin-1-yl)-N-methyl-1H-indole-2-carboxamide;
(S)-4,5-difluoro-N-(8-fluoro-6-oxo-1,4,5,6-tetrahydro-2H-pyrano[3,4-c]isoquinolin-1-yl)-N-methyl-1H-indole-2-carboxamide;
(R)-N-(8,9-difluoro-6-oxo-1,4,5,6-tetrahydro-2H-pyrano[3,4-c]isoquinolin-1-yl)-7-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-7-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-5,6-difluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-4,6-difluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4,5-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-4,5-difluoro-N-methyl-1H-indole-2-carboxamide;
(R)-N-(8,9-difluoro-6-oxo-1,4,5,6-tetrahydro-2H-pyrano[3,4-c]isoquinolin-1-yl)-5,5-difluoro-N-methyl-4,5,6,7-tetrahydro-1H-indole-2-carboxamide;
(S)-N-(8,9-difluoro-6-oxo-1,4,5,6-tetrahydro-2H-pyrano[3,4-c]isoquinolin-1-yl)-5,5-difluoro-N-methyl-4,5,6,7-tetrahydro-1H-indole-2-carboxamide;
(R)-5,6-difluoro-N-(8-fluoro-6-oxo-1,4,5,6-tetrahydro-2H-pyrano[3,4-c]isoquinolin-1-yl)-N-methyl-1H-indole-2-carboxamide;
(S)-5,6-difluoro-N-(8-fluoro-6-oxo-1,4,5,6-tetrahydro-2H-pyrano[3,4-c]isoquinolin-1-yl)-N-methyl-1H-indole-2-carboxamide;
(R)-6-chloro-N-(8,9-difluoro-6-oxo-1,4,5,6-tetrahydro-2H-pyrano[3,4-c]isoquinolin-1-yl)-N-methylimidazo[1,2-a]pyridine-2-carboxamide;
(S)-6-chloro-N-(8,9-difluoro-6-oxo-1,4,5,6-tetrahydro-2H-pyrano[3,4-c]isoquinolin-1-yl)-N-methylimidazo[1,2-a]pyridine-2-carboxamide;
(R)-6-chloro-N-(8-fluoro-6-oxo-1,4,5,6-tetrahydro-2H-pyrano[3,4-c]isoquinolin-1-yl)-N-methylimidazo[1,2-a]pyridine-2-carboxamide;
(S)-6-chloro-N-(8-fluoro-6-oxo-1,4,5,6-tetrahydro-2H-pyrano[3,4-c]isoquinolin-1-yl)-N-methylimidazo[1,2-a]pyridine-2-carboxamide;
(R)-6-chloro-N-(8-fluoro-6-oxo-1,4,5,6-tetrahydro-2H-pyrano[3,4-c]isoquinolin-1-yl)-N-ethylimidazo[1,2-a]pyridine-2-carboxamide;
(S)-6-chloro-N-(8-fluoro-6-oxo-1,4,5,6-tetrahydro-2H-pyrano[3,4-c]isoquinolin-1-yl)-N-ethylimidazo[1,2-a]pyridine-2-carboxamide;
(R)-N-(8,9-difluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-(1R)-yl)-N,3,3-trimethylindoline-(2R)-carboxamide;
N-(8,9-difluoro-6-oxo-1,4,5,6-tetrahydro-2H-pyrano[3,4-c]isoquinolin-(1R)-yl)-N,3,3-trimethylindoline-(2S)-carboxamide;
N-(8,9-difluoro-6-oxo-1,4,5,6-tetrahydro-2H-pyrano[3,4-c]isoquinolin-(1S)-yl)-N,3,3-trimethylindoline-(2R)-carboxamide;
N-(8,9-difluoro-6-oxo-1,4,5,6-tetrahydro-2H-pyrano[3,4-c]isoquinolin-(1S)-yl)-N,3,3-trimethylindoline-(2S)-carboxamide;
(R)-N-(8-fluoro-3-methyl-6-oxo-1,2,3,4,5,6-hexahydrobenzo[c][1,7]naphthyridin-1-yl)-N-methyl-1H-indole-2-carboxamide;
(S)-N-(8-fluoro-3-methyl-6-oxo-1,2,3,4,5,6-hexahydrobenzo[c][1,7]naphthyridin-1-yl)-N-methyl-1H-indole-2-carboxamide;
(R)-N-(8,9-difluoro-3-methyl-6-oxo-1,2,3,4,5,6-hexahydrobenzo[c][1,7]naphthyridin-1-yl)-N-methyl-1H-indole-2-carboxamide;
(S)-N-(8,9-difluoro-3-methyl-6-oxo-1,2,3,4,5,6-hexahydrobenzo[c][1,7]naphthyridin-1-yl)-N-methyl-1H-indole-2-carboxamide;
(R)-N-(8,9-difluoro-6-oxo-1,4,5,6-tetrahydro-2H-pyrano[3,4-c]isoquinolin-1-yl)-N-methylpyrazolo[1,5-a]pyridine-2-carboxamide;
(S)-N-(8,9-difluoro-6-oxo-1,4,5,6-tetrahydro-2H-pyrano[3,4-c]isoquinolin-1-yl)-N-methylpyrazolo[1,5-a]pyridine-2-carboxamide;
(R)-N-(8,9-difluoro-6-oxo-1,4,5,6-tetrahydro-2H-pyrano[3,4-c]isoquinolin-1-yl)-N-methylpyrrolo[1,2-b]pyridazine-6-carboxamide;
(S)-N-(8,9-difluoro-6-oxo-1,4,5,6-tetrahydro-2H-pyrano[3,4-c]isoquinolin-1-yl)-N-methylpyrrolo[1,2-b]pyridazine-6-carboxamide;
(R)-N-(8,9-difluoro-6-oxo-1,4,5,6-tetrahydro-2H-pyrano[3,4-c]isoquinolin-1-yl)-N-methylquinoline-7-carboxamide;
(S)-N-(8,9-difluoro-6-oxo-1,4,5,6-tetrahydro-2H-pyrano[3,4-c]isoquinolin-1-yl)-N-methylquinoline-7-carboxamide;
(R)-N-(8,9-difluoro-6-oxo-1,4,5,6-tetrahydro-2H-pyrano[3,4-c]isoquinolin-1-yl)-N-methylquinoline-6-carboxamide;
(S)-N-(8,9-difluoro-6-oxo-1,4,5,6-tetrahydro-2H-pyrano[3,4-c]isoquinolin-1-yl)-N-methylquinoline-6-carboxamide;
(R)-N-(3-Acetyl-8-fluoro-6-oxo-1,2,3,4,5,6-hexahydrobenzo[c][1,7]naphthyridin-1-yl)-N-methyl-1H-indole-2-carboxamide;
(S)-N-(3-Acetyl-8-fluoro-6-oxo-1,2,3,4,5,6-hexahydrobenzo[c][1,7]naphthyridin-1-yl)-N-methyl-1H-indole-2-carboxamide;
(R)-N-(8,9-Difluoro-6-oxo-1,4,5,6-tetrahydro-2H-pyrano[3,4-c]isoquinolin-1-yl)-N-methyl-[1,2,4]triazolo[4,3-a]pyridine-6-carboxamide;
(S)-N-(8,9-Difluoro-6-oxo-1,4,5,6-tetrahydro-2H-pyrano[3,4-c]isoquinolin-1-yl)-N-methyl-[1,2,4]triazolo[4,3-a]pyridine-6-carboxamide;
(R)-N-(8,9-Difluoro-6-oxo-1,4,5,6-tetrahydro-2H-pyrano[3,4-c]isoquinolin-1-yl)-N-methylquinoline-3-carboxamide;
(S)-N-(8,9-Difluoro-6-oxo-1,4,5,6-tetrahydro-2H-pyrano[3,4-c]isoquinolin-1-yl)-N-methylquinoline-3-carboxamide;
(R)-N-(8,9-Difluoro-6-oxo-1,4,5,6-tetrahydro-2H-pyrano[3,4-c]isoquinolin-1-yl)-N-methylquinoxaline-6-carboxamide;
(S)-N-(8,9-Difluoro-6-oxo-1,4,5,6-tetrahydro-2H-pyrano[3,4-c]isoquinolin-1-yl)-N-methylquinoxaline-6-carboxamide;
(R)-N-(8,9-Difluoro-6-oxo-1,4,5,6-tetrahydro-2H-pyrano[3,4-c]isoquinolin-1-yl)-N-methyl-6-(trifluoromethyl)nicotinamide;
(S)-N-(8,9-Difluoro-6-oxo-1,4,5,6-tetrahydro-2H-pyrano[3,4-c]isoquinolin-1-yl)-N-methyl-6-(trifluoromethyl)nicotinamide;
(R)-N-(8,9-Difluoro-6-oxo-1,4,5,6-tetrahydro-2H-pyrano[3,4-c]isoquinolin-1-yl)-3-fluoro-N-methyl-4-(trifluoromethyl)benzamide;
(S)-N-(8,9-Difluoro-6-oxo-1,4,5,6-tetrahydro-2H-pyrano[3,4-c]isoquinolin-1-yl)-3-fluoro-N-methyl-4-(trifluoromethyl)benzamide;
(R)-4-Chloro-N-(8,9-difluoro-6-oxo-1,4,5,6-tetrahydro-2H-pyrano[3,4-c]isoquinolin-1-yl)-3-fluoro-N-methylbenzamide;
(S)-4-Chloro-N-(8,9-difluoro-6-oxo-1,4,5,6-tetrahydro-2H-pyrano[3,4-c]isoquinolin-1-yl)-3-fluoro-N-methylbenzamide;
(R)-N-(8,9-Difluoro-6-oxo-1,4,5,6-tetrahydro-2H-pyrano[3,4-c]isoquinolin-1-yl)-3,4,5-trifluoro-N-methylbenzamide;
(S)-N-(8,9-Difluoro-6-oxo-1,4,5,6-tetrahydro-2H-pyrano[3,4-c]isoquinolin-1-yl)-3,4,5-trifluoro-N-methylbenzamide;
(R)-N-(8-Fluoro-6-oxo-1,2,3,4,5,6-hexahydrobenzo[c][1,7]naphthyridin-1-yl)-N-methyl-1H-indole-2-carboxamide;
(S)-N-(8-Fluoro-6-oxo-1,2,3,4,5,6-hexahydrobenzo[c][1,7]naphthyridin-1-yl)-N-methyl-1H-indole-2-carboxamide;
(R)-N-(8-Fluoro-3-(2-hydroxyethyl)-6-oxo-1,2,3,4,5,6-hexahydrobenzo[c][1,7]naphthyridin-1-yl)-N-methyl-1H-indole-2-carboxamide;
(S)-N-(8-Fluoro-3-(2-hydroxyethyl)-6-oxo-1,2,3,4,5,6-hexahydrobenzo[c][1,7]naphthyridin-1-yl)-N-methyl-1H-indole-2-carboxamide;
(R)-N-(3-Acetyl-8,9-difluoro-6-oxo-1,2,3,4,5,6-hexahydrobenzo[c][1,7]naphthyridin-1-yl)-N-methyl-1H-indole-2-carboxamide;
(S)-N-(3-Acetyl-8,9-difluoro-6-oxo-1,2,3,4,5,6-hexahydrobenzo[c][1,7]naphthyridin-1-yl)-N-methyl-1H-indole-2-carboxamide;
(R)-N-(8,9-Difluoro-6-oxo-1,4,5,6-tetrahydro-2H-pyrano[3,4-c]isoquinolin-1-yl)-5-fluoro-N-methylnicotinamide;
(S)-N-(8,9-Difluoro-6-oxo-1,4,5,6-tetrahydro-2H-pyrano[3,4-c]isoquinolin-1-yl)-5-fluoro-N-methylnicotinamide;
(R)-5-Chloro-N-(8,9-difluoro-6-oxo-1,4,5,6-tetrahydro-2H-pyrano[3,4-c]isoquinolin-1-yl)-N-methylnicotinamide;
(S)-5-Chloro-N-(8,9-difluoro-6-oxo-1,4,5,6-tetrahydro-2H-pyrano[3,4-c]isoquinolin-1-yl)-N-methylnicotinamide;
(R)-N-(8,9-Difluoro-6-oxo-1,4,5,6-tetrahydro-2H-pyrano[3,4-c]isoquinolin-1-yl)-2-fluoro-N-methylisonicotinamide;
(S)-N-(8,9-Difluoro-6-oxo-1,4,5,6-tetrahydro-2H-pyrano[3,4-c]isoquinolin-1-yl)-2-fluoro-N-methylisonicotinamide;
(R)-N-(8,9-Difluoro-6-oxo-1,4,5,6-tetrahydro-2H-pyrano[3,4-c]isoquinolin-1-yl)-3-(difluoromethyl)-N-methylbenzamide;
(S)-N-(8,9-Difluoro-6-oxo-1,4,5,6-tetrahydro-2H-pyrano[3,4-c]isoquinolin-1-yl)-3-(difluoromethyl)-N-methylbenzamide;
N-((S)-8,9-Difluoro-6-oxo-1,4,5,6-tetrahydro-2H-pyrano[3,4-c]isoquinolin-1-yl)-(2R)-hydroxy-N-methyl-3-phenylpropanamide;
N-((S)-8,9-Difluoro-6-oxo-1,4,5,6-tetrahydro-2H-pyrano[3,4-c]isoquinolin-1-yl)-(2S)-hydroxy-N-methyl-3-phenylpropanamide;
(R)-N-(8,9-Difluoro-3-(2-hydroxyethyl)-6-oxo-1,2,3,4,5,6-hexahydrobenzo[c][1,7]naphthyridin-1-yl)-N-methyl-1H-indole-2-carboxamide;
(S)-N-(8,9-Difluoro-3-(2-hydroxyethyl)-6-oxo-1,2,3,4,5,6-hexahydrobenzo[c][1,7]naphthyridin-1-yl)-N-methyl-1H-indole-2-carboxamide;
(R)-N-(8,9-Difluoro-6-oxo-1,4,5,6-tetrahydro-2H-pyrano[3,4-c]isoquinolin-1-yl)-N-methylbenzamide;
(S)-N-(8,9-Difluoro-6-oxo-1,4,5,6-tetrahydro-2H-pyrano[3,4-c]isoquinolin-1-yl)-N-methylbenzamide;
(R)-N-(8,9-Difluoro-6-oxo-1,4,5,6-tetrahydro-2H-pyrano[3,4-c]isoquinolin-1-yl)-3,4-difluoro-N-methylbenzamide;
(S)-N-(8,9-Difluoro-6-oxo-1,4,5,6-tetrahydro-2H-pyrano[3,4-c]isoquinolin-1-yl)-3,4-difluoro-N-methylbenzamide;
N-((S)-8,9-Difluoro-6-oxo-1,4,5,6-tetrahydro-2H-pyrano[3,4-c]isoquinolin-1-yl)-(2R)-hydroxy-N-methyl-2-phenylpropanamide;
N-((S)-8,9-Difluoro-6-oxo-1,4,5,6-tetrahydro-2H-pyrano[3,4-c]isoquinolin-1-yl)-(2S)-hydroxy-N-methyl-2-phenylpropanamide;
N-((R)-8,9-Difluoro-6-oxo-1,4,5,6-tetrahydro-2H-pyrano[3,4-c]isoquinolin-1-yl)-(2R)-hydroxy-N-methyl-2-phenylpropanamide;
N-((R)-8,9-Difluoro-6-oxo-1,4,5,6-tetrahydro-2H-pyrano[3,4-c]isoquinolin-1-yl)-(2S)-hydroxy-N-methyl-2-phenylpropanamide; N-((S)-8,9-Difluoro-6-oxo-1,4,5,6-tetrahydro-2H-pyrano[3,4-c]isoquinolin-1-yl)-(2R)-hydroxy-N-methyl-2-phenylacetamide;
N-((S)-8,9-Difluoro-6-oxo-1,4,5,6-tetrahydro-2H-pyrano[3,4-c]isoquinolin-1-yl)-(2S)-hydroxy-N-methyl-2-phenylacetamide;
N-((R)-8,9-Difluoro-6-oxo-1,4,5,6-tetrahydro-2H-pyrano[3,4-c]isoquinolin-1-yl)-(2R)-hydroxy-N-methyl-2-phenylacetamide;
N-((R)-8,9-Difluoro-6-oxo-1,4,5,6-tetrahydro-2H-pyrano[3,4-c]isoquinolin-1-yl)-(2S)-hydroxy-N-methyl-2-phenylacetamide;
(R)-N-(8,9-Difluoro-6-oxo-1,4,5,6-tetrahydro-2H-pyrano[3,4-c]isoquinolin-1-yl)-4,5,6-trifluoro-N-methyl-1H-indole-2-carboxamide;
(S)-N-(8,9-Difluoro-6-oxo-1,4,5,6-tetrahydro-2H-pyrano[3,4-c]isoquinolin-1-yl)-4,5,6-trifluoro-N-methyl-1H-indole-2-carboxamide;
(R)-N-(8,9-Difluoro-6-oxo-1,4,5,6-tetrahydro-2H-pyrano[3,4-c]isoquinolin-1-yl)-N-methyl-4-(trifluoromethyl)benzamide;
(S)-N-(8,9-Difluoro-6-oxo-1,4,5,6-tetrahydro-2H-pyrano[3,4-c]isoquinolin-1-yl)-N-methyl-4-(trifluoromethyl)benzamide;
(R)-4-Chloro-N-(8,9-difluoro-6-oxo-1,4,5,6-tetrahydro-2H-pyrano[3,4-c]isoquinolin-1-yl)-N-methylbenzamide;
(S)-4-Chloro-N-(8,9-difluoro-6-oxo-1,4,5,6-tetrahydro-2H-pyrano[3,4-c]isoquinolin-1-yl)-N-methylbenzamide;
(R)-4-Cyano-N-(8,9-difluoro-6-oxo-1,4,5,6-tetrahydro-2H-pyrano[3,4-c]isoquinolin-1-yl)-3-fluoro-N-methylbenzamide;
(S)-4-Cyano-N-(8,9-difluoro-6-oxo-1,4,5,6-tetrahydro-2H-pyrano[3,4-c]isoquinolin-1-yl)-3-fluoro-N-methylbenzamide;
(R)-N-(8,9-Difluoro-6-oxo-1,2,3,4,5,6-hexahydrobenzo[c][1,7]naphthyridin-1-yl)-5-fluoro-N-methyl-1H-indole-2-carboxamide;
(S)-N-(8,9-Difluoro-6-oxo-1,2,3,4,5,6-hexahydrobenzo[c][1,7]naphthyridin-1-yl)-5-fluoro-N-methyl-1H-indole-2-carboxamide;
(R)-N-(8,9-Difluoro-6-oxo-1,4,5,6-tetrahydro-2H-thiopyrano[3,4-c]isoquinolin-1-yl)-8-fluoro-N-methylindolizine-2-carboxamide;
(S)-N-(8,9-Difluoro-6-oxo-1,4,5,6-tetrahydro-2H-thiopyrano[3,4-c]isoquinolin-1-yl)-8-fluoro-N-methylindolizine-2-carboxamide;
(R)-N-(8,9-Difluoro-6-oxo-1,2,3,4,5,6-hexahydrobenzo[c][1,7]naphthyridin-1-yl)-4-fluoro-N-methyl-1H-indole-2-carboxamide;
(S)-N-(8,9-Difluoro-6-oxo-1,2,3,4,5,6-hexahydrobenzo[c][1,7]naphthyridin-1-yl)-4-fluoro-N-methyl-1H-indole-2-carboxamide;
(R)-N-(8,9-Difluoro-6-oxo-1,2,3,4,5,6-hexahydrobenzo[c][1,7]naphthyridin-1-yl)-6-fluoro-N-methyl-1H-indole-2-carboxamide;
(S)-N-(8,9-Difluoro-6-oxo-1,2,3,4,5,6-hexahydrobenzo[c][1,7]naphthyridin-1-yl)-6-fluoro-N-methyl-1H-indole-2-carboxamide;
(R)-N-(8,9-Difluoro-6-oxo-1,4,5,6-tetrahydro-2H-pyrano[3,4-c]isoquinolin-1-yl)-3-(difluoromethyl)-4-fluoro-N-methylbenzamide;
(S)-N-(8,9-Difluoro-6-oxo-1,4,5,6-tetrahydro-2H-pyrano[3,4-c]isoquinolin-1-yl)-3-(difluoromethyl)-4-fluoro-N-methylbenzamide;
(R)-N-(8,9-Difluoro-6-oxo-1,4,5,6-tetrahydro-2H-pyrano[3,4-c]isoquinolin-1-yl)-4-(difluoromethyl)-3-fluoro-N-methylbenzamide;
(S)-N-(8,9-Difluoro-6-oxo-1,4,5,6-tetrahydro-2H-pyrano[3,4-c]isoquinolin-1-yl)-4-(difluoromethyl)-3-fluoro-N-methylbenzamide;
(R)-N-(8,9-Difluoro-6-oxo-1,4,5,6-tetrahydro-2H-pyrano[3,4-c]isoquinolin-1-yl)-4-(difluoromethyl)-N-methylbenzamide;
(S)-N-(8,9-Difluoro-6-oxo-1,4,5,6-tetrahydro-2H-pyrano[3,4-c]isoquinolin-1-yl)-4-(difluoromethyl)-N-methylbenzamide;
(R)-3-Chloro-N-(8,9-difluoro-6-oxo-1,4,5,6-tetrahydro-2H-pyrano[3,4-c]isoquinolin-1-yl)-4-fluoro-N-methylbenzamide;
(S)-3-Chloro-N-(8,9-difluoro-6-oxo-1,4,5,6-tetrahydro-2H-pyrano[3,4-c]isoquinolin-1-yl)-4-fluoro-N-methylbenzamide;
(R)-N-(8,9-Difluoro-6-oxo-1,2,3,4,5,6-hexahydrobenzo[c][1,7]naphthyridin-1-yl)-7-fluoro-N-methyl-1H-indole-2-carboxamide;
(S)-N-(8,9-Difluoro-6-oxo-1,2,3,4,5,6-hexahydrobenzo[c][1,7]naphthyridin-1-yl)-7-fluoro-N-methyl-1H-indole-2-carboxamide;
(R)-N-(8,9-Difluoro-6-oxo-1,2,3,4,5,6-hexahydrobenzo[c][1,7]naphthyridin-1-yl)-5,6-difluoro-N-methyl-1H-indole-2-carboxamide;
(S)-N-(8,9-Difluoro-6-oxo-1,2,3,4,5,6-hexahydrobenzo[c][1,7]naphthyridin-1-yl)-5,6-difluoro-N-methyl-1H-indole-2-carboxamide;
(R)-N-(8,9-Difluoro-6-oxo-1,2,3,4,5,6-hexahydrobenzo[c][1,7]naphthyridin-1-yl)-4,6-difluoro-N-methyl-1H-indole-2-carboxamide;
(S)-N-(8,9-Difluoro-6-oxo-1,2,3,4,5,6-hexahydrobenzo[c][1,7]naphthyridin-1-yl)-4,6-difluoro-N-methyl-1H-indole-2-carboxamide;
(R)-N-(8,9-Difluoro-6-oxo-1,2,3,4,5,6-hexahydrobenzo[c][1,7]naphthyridin-1-yl)-4,5-difluoro-N-methyl-1H-indole-2-carboxamide;
(S)-N-(8,9-Difluoro-6-oxo-1,2,3,4,5,6-hexahydrobenzo[c][1,7]naphthyridin-1-yl)-4,5-difluoro-N-methyl-1H-indole-2-carboxamide;
(R)-6-Chloro-N-(8,9-difluoro-6-oxo-1,2,3,4,5,6-hexahydrobenzo[c][1,7]naphthyridin-1-yl)-N-methylindolizine-2-carboxamide;
(S)-6-Chloro-N-(8,9-difluoro-6-oxo-1,2,3,4,5,6-hexahydrobenzo[c][1,7]naphthyridin-1-yl)-N-methylindolizine-2-carboxamide;
(R)-N-(8,9-Difluoro-6-oxo-1,4,5,6-tetrahydro-2H-pyrano[3,4-c]isoquinolin-1-yl)-N-methylbenzo[d]thiazole-2-carboxamide;
(S)-N-(8,9-Difluoro-6-oxo-1,4,5,6-tetrahydro-2H-pyrano[3,4-c]isoquinolin-1-yl)-N-methylbenzo[d]thiazole-2-carboxamide;
(R)-N-(8,9-Difluoro-6-oxo-1,4,5,6-tetrahydro-2H-pyrano[3,4-c]isoquinolin-1-yl)-N-methylbenzo[d]oxazole-2-carboxamide;
(S)-N-(8,9-Difluoro-6-oxo-1,4,5,6-tetrahydro-2H-pyrano[3,4-c]isoquinolin-1-yl)-N-methylbenzo[d]oxazole-2-carboxamide;
(R)-N-(8,9-Difluoro-6-oxo-1,4,5,6-tetrahydro-2H-pyrano[3,4-c]isoquinolin-1-yl)-N-methyl-3-(trifluoromethyl)-1H-pyrazole-5-carboxamide;
(S)-N-(8,9-Difluoro-6-oxo-1,4,5,6-tetrahydro-2H-pyrano[3,4-c]isoquinolin-1-yl)-N-methyl-3-(trifluoromethyl)-1H-pyrazole-5-carboxamide;
(R)-N-(8,9-Difluoro-6-oxo-1,4,5,6-tetrahydro-2H-pyrano[3,4-c]isoquinolin-1-yl)-3-(4-fluorophenyl)-N-methyl-1H-pyrazole-5-carboxamide;
(S)-N-(8,9-Difluoro-6-oxo-1,4,5,6-tetrahydro-2H-pyrano[3,4-c]isoquinolin-1-yl)-3-(4-fluorophenyl)-N-methyl-1H-pyrazole-5-carboxamide;
(R)-N-(8,9-Difluoro-6-oxo-1,4,5,6-tetrahydro-2H-pyrano[3,4-c]isoquinolin-1-yl)-3-fluoro-N-methylbenzamide;
(S)-N-(8,9-Difluoro-6-oxo-1,4,5,6-tetrahydro-2H-pyrano[3,4-c]isoquinolin-1-yl)-3-fluoro-N-methylbenzamide;
(R)-3-Chloro-N-(8,9-difluoro-6-oxo-1,4,5,6-tetrahydro-2H-pyrano[3,4-c]isoquinolin-1-yl)-N-methylbenzamide;
(S)-3-Chloro-N-(8,9-difluoro-6-oxo-1,4,5,6-tetrahydro-2H-pyrano[3,4-c]isoquinolin-1-yl)-N-methylbenzamide;
(R)-3-Bromo-N-(8,9-difluoro-6-oxo-1,4,5,6-tetrahydro-2H-pyrano[3,4-c]isoquinolin-1-yl)-N-methylbenzamide;
(S)-3-Bromo-N-(8,9-difluoro-6-oxo-1,4,5,6-tetrahydro-2H-pyrano[3,4-c]isoquinolin-1-yl)-N-methylbenzamide;
(R)-3-Bromo-N-(8,9-difluoro-6-oxo-1,4,5,6-tetrahydro-2H-pyrano[3,4-c]isoquinolin-1-yl)-4-fluoro-N-methylbenzamide;
(S)-3-Bromo-N-(8,9-difluoro-6-oxo-1,4,5,6-tetrahydro-2H-pyrano[3,4-c]isoquinolin-1-yl)-4-fluoro-N-methylbenzamide;
(R)-4-Bromo-N-(8,9-difluoro-6-oxo-1,4,5,6-tetrahydro-2H-pyrano[3,4-c]isoquinolin-1-yl)-N-methylbenzamide;
(S)-4-Bromo-N-(8,9-difluoro-6-oxo-1,4,5,6-tetrahydro-2H-pyrano[3,4-c]isoquinolin-1-yl)-N-methylbenzamide;
(R)-3-Fluoro-N-methyl-N-(6-oxo-1,4,5,6-tetrahydro-2H-pyrano[3,4-c]isoquinolin-1-yl)-4-(trifluoromethyl)benzamide;
(S)-3-Fluoro-N-methyl-N-(6-oxo-1,4,5,6-tetrahydro-2H-pyrano[3,4-c]isoquinolin-1-yl)-4-(trifluoromethyl)benzamide;
(R)-3-Fluoro-N-methyl-N-(4-oxo-4,5,8,9-tetrahydro-6H-pyrano[3,4-b]thieno[3,4-d]pyridin-9-yl)-4-(trifluoromethyl)benzamide;
(S)-3-Fluoro-N-methyl-N-(4-oxo-4,5,8,9-tetrahydro-6H-pyrano[3,4-b]thieno[3,4-d]pyridin-9-yl)-4-(trifluoromethyl)benzamide;
(R)-N-(8-Cyano-6-oxo-1,4,5,6-tetrahydro-2H-pyrano[3,4-c]isoquinolin-1-yl)-3-fluoro-N-methyl-4-(trifluoromethyl)benzamide;
(S)-N-(8-Cyano-6-oxo-1,4,5,6-tetrahydro-2H-pyrano[3,4-c]isoquinolin-1-yl)-3-fluoro-N-methyl-4-(trifluoromethyl)benzamide;
(R)-3-Cyano-N-(8,9-difluoro-6-oxo-1,4,5,6-tetrahydro-2H-pyrano[3,4-c]isoquinolin-1-yl)-N-methylbenzamide;
(S)-3-Cyano-N-(8,9-difluoro-6-oxo-1,4,5,6-tetrahydro-2H-pyrano[3,4-c]isoquinolin-1-yl)-N-methylbenzamide;
(R)-N-(8,9-Difluoro-6-oxo-1,4,5,6-tetrahydro-2H-pyrano[3,4-c]isoquinolin-1-yl)-3-fluoro-N-methyl-4-(trifluoromethoxy)benzamide;
(S)-N-(8,9-Difluoro-6-oxo-1,4,5,6-tetrahydro-2H-pyrano[3,4-c]isoquinolin-1-yl)-3-fluoro-N-methyl-4-(trifluoromethoxy)benzamide;
(R)-N-(8,9-Difluoro-6-oxo-1,4,5,6-tetrahydro-2H-pyrano[3,4-c]isoquinolin-1-yl)-3-fluoro-N,4-dimethylbenzamide;
(S)-N-(8,9-Difluoro-6-oxo-1,4,5,6-tetrahydro-2H-pyrano[3,4-c]isoquinolin-1-yl)-3-fluoro-N,4-dimethylbenzamide;
2-(3-Chlorophenyl)-N-((S)-8,9-difluoro-6-oxo-1,4,5,6-tetrahydro-2H-pyrano[3,4-c]isoquinolin-1-yl)-(2R)-hydroxy-N-methylacetamide;
2-(3-Chlorophenyl)-N-((S)-8,9-difluoro-6-oxo-1,4,5,6-tetrahydro-2H-pyrano[3,4-c]isoquinolin-1-yl)-(2S)-hydroxy-N-methylacetamide;
(R)-N-(8,9-Difluoro-6-oxo-1,4,5,6-tetrahydro-2H-pyrano[3,4-c]isoquinolin-1-yl)-3,5-difluoro-N-methyl-4-(trifluoromethyl)benzamide;
(S)-N-(8,9-Difluoro-6-oxo-1,4,5,6-tetrahydro-2H-pyrano[3,4-c]isoquinolin-1-yl)-3,5-difluoro-N-methyl-4-(trifluoromethyl)benzamide;
(R)-5-Chloro-N-(8,9-difluoro-6-oxo-1,4,5,6-tetrahydro-2H-pyrano[3,4-c]isoquinolin-1-yl)-N-methylthiophene-3-carboxamide;
(S)-5-Chloro-N-(8,9-difluoro-6-oxo-1,4,5,6-tetrahydro-2H-pyrano[3,4-c]isoquinolin-1-yl)-N-methylthiophene-3-carboxamide;
(2R)-Amino-N-((R)-8,9-difluoro-6-oxo-1,4,5,6-tetrahydro-2H-pyrano[3,4-c]isoquinolin-1-yl)-(3R)-hydroxy-N-methylbutanamide;
(2R)-Amino-N-((R)-8,9-difluoro-6-oxo-1,4,5,6-tetrahydro-2H-pyrano[3,4-c]isoquinolin-1-yl)-(3S)-hydroxy-N-methylbutanamide;
(2S)-Amino-N-((R)-8,9-difluoro-6-oxo-1,4,5,6-tetrahydro-2H-pyrano[3,4-c]isoquinolin-1-yl)-(3R)-hydroxy-N-methylbutanamide;
(2S)-Amino-N-((R)-8,9-difluoro-6-oxo-1,4,5,6-tetrahydro-2H-pyrano[3,4-c]isoquinolin-1-yl)-(3S)-hydroxy-N-methylbutanamide;
(2R)-Amino-N-((S)-8,9-difluoro-6-oxo-1,4,5,6-tetrahydro-2H-pyrano[3,4-c]isoquinolin-1-yl)-(3R)-hydroxy-N-methylbutanamide;
(2R)-Amino-N-((S)-8,9-difluoro-6-oxo-1,4,5,6-tetrahydro-2H-pyrano[3,4-c]isoquinolin-1-yl)-(3S)-hydroxy-N-methylbutanamide;
(2S)-Amino-N-((S)-8,9-difluoro-6-oxo-1,4,5,6-tetrahydro-2H-pyrano[3,4-c]isoquinolin-1-yl)-(3R)-hydroxy-N-methylbutanamide;
(2S)-Amino-N-((S)-8,9-difluoro-6-oxo-1,4,5,6-tetrahydro-2H-pyrano[3,4-c]isoquinolin-1-yl)-(3S)-hydroxy-N-methylbutanamide;
(R)-3-Chloro-N-(8,9-difluoro-6-oxo-1,4,5,6-tetrahydro-2H-pyrano[3,4-c]isoquinolin-1-yl)-4-fluoro-N-methylbenzenesulfonamide;
(S)-3-Chloro-N-(8,9-difluoro-6-oxo-1,4,5,6-tetrahydro-2H-pyrano[3,4-c]isoquinolin-1-yl)-4-fluoro-N-methylbenzenesulfonamide;
(R)-3-Chloro-N-(8,9-difluoro-6-oxo-1,4,5,6-tetrahydro-2H-pyrano[3,4-c]isoquinolin-1-yl)-N-methylbenzenesulfonamide;
(S)-3-Chloro-N-(8,9-difluoro-6-oxo-1,4,5,6-tetrahydro-2H-pyrano[3,4-c]isoquinolin-1-yl)-N-methylbenzenesulfonamide;
(R)-3-Cyano-N-(8,9-difluoro-6-oxo-1,4,5,6-tetrahydro-2H-pyrano[3,4-c]isoquinolin-1-yl)-N-methylbenzenesulfonamide;
(S)-3-Cyano-N-(8,9-difluoro-6-oxo-1,4,5,6-tetrahydro-2H-pyrano[3,4-c]isoquinolin-1-yl)-N-methylbenzenesulfonamide;
(R)-3-Cyano-N-(8,9-difluoro-6-oxo-1,4,5,6-tetrahydro-2H-pyrano[3,4-c]isoquinolin-1-yl)-4-fluoro-N-methylbenzenesulfonamide;
(S)-3-Cyano-N-(8,9-difluoro-6-oxo-1,4,5,6-tetrahydro-2H-pyrano[3,4-c]isoquinolin-1-yl)-4-fluoro-N-methylbenzenesulfonamide;
(R)-N-(8,9-Difluoro-6-oxo-1,4,5,6-tetrahydro-2H-pyrano[3,4-c]isoquinolin-1-yl)-4-ethyl-3-fluoro-N-methylbenzamide;
(S)-N-(8,9-Difluoro-6-oxo-1,4,5,6-tetrahydro-2H-pyrano[3,4-c]isoquinolin-1-yl)-4-ethyl-3-fluoro-N-methylbenzamide;
(S)-N-(8,9-difluoro-6-oxo-1,4,5,6-tetrahydro-2H-pyrano[3,4-c]isoquinolin-1-yl)indolizine-2-carboxamide;
(R)-N-(8,9-difluoro-6-oxo-1,4,5,6-tetrahydro-2H-pyrano[3,4-c]isoquinolin-1-yl)indolizine-2-carboxamide;
(R,R)-N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
(R,S)-N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
(S,R)-N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
(S,S)-N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
(S)-N-(8,9-difluoro-3,3-dioxido-6-oxo-1,4,5,6-tetrahydro-2H-thiopyrano[3,4-c]isoquinolin-1-yl)-8-fluoro-N-methylindolizine-2-carboxamide;
(R)-N-(8,9-difluoro-3,3-dioxido-6-oxo-1,4,5,6-tetrahydro-2H-thiopyrano[3,4-c]isoquinolin-1-yl)-8-fluoro-N-methylindolizine-2-carboxamide;
(S)-N-(8,9-difluoro-6-oxo-1,4,5,6-tetrahydro-2H-pyrano[3,4-c]isoquinolin-1-yl)-4-(difluoromethoxy)-3-fluoro-N-methylbenzamide;
(R)-N-(8,9-difluoro-6-oxo-1,4,5,6-tetrahydro-2H-pyrano[3,4-c]isoquinolin-1-yl)-4-(difluoromethoxy)-3-fluoro-N-methylbenzamide;
(S)-N-(8,9-difluoro-6-oxo-1,4,5,6-tetrahydro-2H-pyrano[3,4-c]isoquinolin-1-yl)-4-(2-hydroxypropan-2-yl)-N-methylbenzamide;
(R)-N-(8,9-difluoro-6-oxo-1,4,5,6-tetrahydro-2H-pyrano[3,4-c]isoquinolin-1-yl)-4-(2-hydroxypropan-2-yl)-N-methylbenzamide;
(S)-N-(8,9-difluoro-6-oxo-1,4,5,6-tetrahydro-2H-pyrano[3,4-c]isoquinolin-1-yl)-3-(2-hydroxypropan-2-yl)-N-methylbenzamide;
(R)-N-(8,9-difluoro-6-oxo-1,4,5,6-tetrahydro-2H-pyrano[3,4-c]isoquinolin-1-yl)-3-(2-hydroxypropan-2-yl)-N-methylbenzamide;
(S)-N-(8,9-difluoro-6-oxo-1,4,5,6-tetrahydro-2H-pyrano[3,4-c]isoquinolin-1-yl)-5,6-difluoro-1H-indole-2-carboxamide;
(R)-N-(8,9-difluoro-6-oxo-1,4,5,6-tetrahydro-2H-pyrano[3,4-c]isoquinolin-1-yl)-5,6-difluoro-1H-indole-2-carboxamide;
(S)-N-(8,9-difluoro-6-oxo-1,4,5,6-tetrahydro-2H-pyrano[3,4-c]isoquinolin-1-yl)-4,6-difluoro-1H-indole-2-carboxamide;
(R)-N-(8,9-difluoro-6-oxo-1,4,5,6-tetrahydro-2H-pyrano[3,4-c]isoquinolin-1-yl)-4,6-difluoro-1H-indole-2-carboxamide;
(S)-N-(8,9-difluoro-6-oxo-1,4,5,6-tetrahydro-2H-pyrano[3,4-c]isoquinolin-1-yl)-3-fluoro-4-(trifluoromethyl)benzamide;
(R)-N-(8,9-difluoro-6-oxo-1,4,5,6-tetrahydro-2H-pyrano[3,4-c]isoquinolin-1-yl)-3-fluoro-4-(trifluoromethyl)benzamide;
(S)-4-chloro-N-(8,9-difluoro-6-oxo-1,4,5,6-tetrahydro-2H-pyrano[3,4-c]isoquinolin-1-yl)-3-fluorobenzamide;
(R)-4-chloro-N-(8,9-difluoro-6-oxo-1,4,5,6-tetrahydro-2H-pyrano[3,4-c]isoquinolin-1-yl)-3-fluorobenzamide;
(S)-N-(8,9-difluoro-6-oxo-1,2,3,4,5,6-hexahydrobenzo[c][1,7]naphthyridin-1-yl)-8-fluoro-N-methylindolizine-2-carboxamide;
(R)-N-(8,9-difluoro-6-oxo-1,2,3,4,5,6-hexahydrobenzo[c][1,7]naphthyridin-1-yl)-8-fluoro-N-methylindolizine-2-carboxamide;
(S)-N-(8,9-difluoro-6-oxo-1,2,3,4,5,6-hexahydrobenzo[c][1,7]naphthyridin-1-yl)-7-fluoro-N-methylindolizine-2-carboxamide;
(R)-N-(8,9-difluoro-6-oxo-1,2,3,4,5,6-hexahydrobenzo[c][1,7]naphthyridin-1-yl)-7-fluoro-N-methylindolizine-2-carboxamide;
(S)-4-bromo-N-(8,9-difluoro-6-oxo-1,2,3,4,5,6-hexahydrobenzo[c][1,7]naphthyridin-1-yl)-3,5-difluoro-N-methylbenzamide;
(R)-4-bromo-N-(8,9-difluoro-6-oxo-1,2,3,4,5,6-hexahydrobenzo[c][1,7]naphthyridin-1-yl)-3,5-difluoro-N-methylbenzamide;
(S)-4-bromo-3,5-difluoro-N-(8-fluoro-6-oxo-1,4,5,6-tetrahydro-2H-pyrano[3,4-c]isoquinolin-1-yl)-N-methylbenzamide;
(R)-4-bromo-3,5-difluoro-N-(8-fluoro-6-oxo-1,4,5,6-tetrahydro-2H-pyrano[3,4-c]isoquinolin-1-yl)-N-methylbenzamide;
(S)-4-chloro-N-(8,9-difluoro-6-oxo-1,2,3,4,5,6-hexahydrobenzo[c][1,7]naphthyridin-1-yl)-3,5-difluoro-N-methylbenzamide;
(R)-4-chloro-N-(8,9-difluoro-6-oxo-1,2,3,4,5,6-hexahydrobenzo[c][1,7]naphthyridin-1-yl)-3,5-difluoro-N-methylbenzamide;
(S)-4-bromo-3,5-difluoro-N-(8-fluoro-6-oxo-1,2,3,4,5,6-hexahydrobenzo[c][1,7]naphthyridin-1-yl)-N-methylbenzamide;
(R)-4-bromo-3,5-difluoro-N-(8-fluoro-6-oxo-1,2,3,4,5,6-hexahydrobenzo[c][1,7]naphthyridin-1-yl)-N-methylbenzamide;
(S)-4-bromo-N-(8,9-difluoro-6-oxo-1,4,5,6-tetrahydro-2H-pyrano[3,4-c]isoquinolin-1-yl)-3,5-difluoro-N-methylbenzamide;
(R)-4-bromo-N-(8,9-difluoro-6-oxo-1,4,5,6-tetrahydro-2H-pyrano[3,4-c]isoquinolin-1-yl)-3,5-difluoro-N-methylbenzamide;
(S)-4-chloro-N-(8,9-difluoro-6-oxo-1,4,5,6-tetrahydro-2H-pyrano[3,4-c]isoquinolin-1-yl)-3,5-difluoro-N-methylbenzamide;
(R)-4-chloro-N-(8,9-difluoro-6-oxo-1,4,5,6-tetrahydro-2H-pyrano[3,4-c]isoquinolin-1-yl)-3,5-difluoro-N-methylbenzamide;
(S)-4-chloro-3,5-difluoro-N-(8-fluoro-6-oxo-1,4,5,6-tetrahydro-2H-pyrano[3,4-c]isoquinolin-1-yl)-N-methylbenzamide;
(R)-4-chloro-3,5-difluoro-N-(8-fluoro-6-oxo-1,4,5,6-tetrahydro-2H-pyrano[3,4-c]isoquinolin-1-yl)-N-methylbenzamide;
(S)-4-chloro-3,5-difluoro-N-(8-fluoro-6-oxo-1,2,3,4,5,6-hexahydrobenzo[c][1,7]naphthyridin-1-yl)-N-methylbenzamide;
(R)-4-chloro-3,5-difluoro-N-(8-fluoro-6-oxo-1,2,3,4,5,6-hexahydrobenzo[c][1,7]naphthyridin-1-yl)-N-methylbenzamide;
(S)-4-(difluoromethyl)-3,5-difluoro-N-(8-fluoro-6-oxo-1,4,5,6-tetrahydro-2H-pyrano[3,4-c]isoquinolin-1-yl)-N-methylbenzamide;
(R)-4-(difluoromethyl)-3,5-difluoro-N-(8-fluoro-6-oxo-1,4,5,6-tetrahydro-2H-pyrano[3,4-c]isoquinolin-1-yl)-N-methylbenzamide;
(S)-N-(8,9-difluoro-6-oxo-1,4,5,6-tetrahydro-2H-pyrano[3,4-c]isoquinolin-1-yl)-4-(difluoromethyl)-3,5-difluoro-N-methylbenzamide;
(R)-N-(8,9-difluoro-6-oxo-1,4,5,6-tetrahydro-2H-pyrano[3,4-c]isoquinolin-1-yl)-4-(difluoromethyl)-3,5-difluoro-N-methylbenzamide;
(S)-4-(difluoromethyl)-N-(8-fluoro-6-oxo-1,4,5,6-tetrahydro-2H-pyrano[3,4-c]isoquinolin-1-yl)-N-methyl-1H-indole-2-carboxamide;
(R)-4-(difluoromethyl)-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,2,3,4,5,6-hexahydrobenzo[c][1,7]naphthyridin-1-yl)-4-(difluoromethyl)-3,5-difluoro-N-methylbenzamide;
(R)-N-(8,9-difluoro-6-oxo-1,2,3,4,5,6-hexahydrobenzo[c][1,7]naphthyridin-1-yl)-4-(difluoromethyl)-3,5-difluoro-N-methylbenzamide;
(S)-4-(difluoromethyl)-3,5-difluoro-N-(8-fluoro-6-oxo-1,2,3,4,5,6-hexahydrobenzo[c][1,7]naphthyridin-1-yl)-N-methylbenzamide;
(R)-4-(difluoromethyl)-3,5-difluoro-N-(8-fluoro-6-oxo-1,2,3,4,5,6-hexahydrobenzo[c][1,7]naphthyridin-1-yl)-N-methylbenzamide;
N-((1S)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1S,4R)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1S,4S)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1R)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1R,4R)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1R,4S)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-N,1-dimethyl-1H-pyrazole-4-carboxamide;
(R)-N-(8,9-difluoro-6-oxo-1,4,5,6-tetrahydro-2H-pyrano[3,4-c]isoquinolin-1-yl)-N,1-dimethyl-1H-pyrazole-4-carboxamide;
(S)-N-(8,9-difluoro-6-oxo-1,4,5,6-tetrahydro-2H-pyrano[3,4-c]isoquinolin-1-yl)-N-methyl-2-phenylacrylamide;
(R)-N-(8,9-difluoro-6-oxo-1,4,5,6-tetrahydro-2H-pyrano[3,4-c]isoquinolin-1-yl)-N-methyl-2-phenylacrylamide;
(S)-N-(8,9-difluoro-6-oxo-1,4,5,6-tetrahydro-2H-pyrano[3,4-c]isoquinolin-1-yl)-5-fluoro-N-methyl-1H-pyrrolo[2,3-b]pyridine-2-carboxamide;
(R)-N-(8,9-difluoro-6-oxo-1,4,5,6-tetrahydro-2H-pyrano[3,4-c]isoquinolin-1-yl)-5-fluoro-N-methyl-1H-pyrrolo[2,3-b]pyridine-2-carboxamide;
(S)-5-chloro-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
(R)-5-chloro-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
(S)-5-bromo-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
(R)-5-bromo-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
(S)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-3-carboxamide;
(R)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-3-carboxamide;
(S)-5-bromo-N-(8,9-difluoro-6-oxo-1,4,5,6-tetrahydro-2H-pyrano[3,4-c]isoquinolin-1-yl)-N-methylthiophene-3-carboxamide;
(R)-5-bromo-N-(8,9-difluoro-6-oxo-1,4,5,6-tetrahydro-2H-pyrano[3,4-c]isoquinolin-1-yl)-N-methylthiophene-3-carboxamide;
(S)-N-(8,9-difluoro-6-oxo-1,4,5,6-tetrahydro-2H-pyrano[3,4-c]isoquinolin-1-yl)-N-methyl-2,3-dihydro-1H-indene-2-carboxamide;
(R)-N-(8,9-difluoro-6-oxo-1,4,5,6-tetrahydro-2H-pyrano[3,4-c]isoquinolin-1-yl)-N-methyl-2,3-dihydro-1H-indene-2-carboxamide;
(S)-1-(tert-butyl)-N-(8,9-difluoro-6-oxo-1,4,5,6-tetrahydro-2H-pyrano[3,4-c]isoquinolin-1-yl)-N-methyl-1H-pyrazole-4-carboxamide;
(R)-1-(tert-butyl)-N-(8,9-difluoro-6-oxo-1,4,5,6-tetrahydro-2H-pyrano[3,4-c]isoquinolin-1-yl)-N-methyl-1H-pyrazole-4-carboxamide;
(S)-N-(8,9-difluoro-6-oxo-1,4,5,6-tetrahydro-2H-pyrano[3,4-c]isoquinolin-1-yl)-N-methyl-1-(trifluoromethyl)-1H-pyrazole-4-carboxamide;
(R)-N-(8,9-difluoro-6-oxo-1,4,5,6-tetrahydro-2H-pyrano[3,4-c]isoquinolin-1-yl)-N-methyl-1-(trifluoromethyl)-1H-pyrazole-4-carboxamide;
(S)-4-bromo-N-(8,9-difluoro-6-oxo-1,4,5,6-tetrahydro-2H-pyrano[3,4-c]isoquinolin-1-yl)-N-methyl-1H-pyrrole-2-carboxamide;
(R)-4-bromo-N-(8,9-difluoro-6-oxo-1,4,5,6-tetrahydro-2H-pyrano[3,4-c]isoquinolin-1-yl)-N-methyl-1H-pyrrole-2-carboxamide;
(S)-2-amino-2-(4-chlorophenyl)-N-((S)-8,9-difluoro-6-oxo-1,4,5,6-tetrahydro-2H-pyrano[3,4-c]isoquinolin-1-yl)-N-methylacetamide;
(R)-2-amino-2-(4-chlorophenyl)-N-((S)-8,9-difluoro-6-oxo-1,4,5,6-tetrahydro-2H-pyrano[3,4-c]isoquinolin-1-yl)-N-methylacetamide;
(S)-2-amino-2-(4-chlorophenyl)-N-((R)-8,9-difluoro-6-oxo-1,4,5,6-tetrahydro-2H-pyrano[3,4-c]isoquinolin-1-yl)-N-methylacetamide;
(R)-2-amino-2-(4-chlorophenyl)-N-((R)-8,9-difluoro-6-oxo-1,4,5,6-tetrahydro-2H-pyrano[3,4-c]isoquinolin-1-yl)-N-methylacetamide;
2-amino-2-(4-chlorophenyl)-N-((S)-8,9-difluoro-6-oxo-1,4,5,6-tetrahydro-2H-pyrano[3,4-c]isoquinolin-1-yl)-N-methylacetamide;
2-amino-2-(4-chlorophenyl)-N-((S)-8,9-difluoro-6-oxo-1,4,5,6-tetrahydro-2H-pyrano[3,4-c]isoquinolin-1-yl)-N-methylacetamide;
(S)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
(R)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
(S)-2-amino-2-(3-chlorophenyl)-N-((S)-8,9-difluoro-6-oxo-1,4,5,6-tetrahydro-2H-pyrano[3,4-c]isoquinolin-1-yl)-N-methylacetamide;
(R)-2-amino-2-(3-chlorophenyl)-N-((S)-8,9-difluoro-6-oxo-1,4,5,6-tetrahydro-2H-pyrano[3,4-c]isoquinolin-1-yl)-N-methylacetamide;
(S)-2-amino-2-(3-chlorophenyl)-N-((R)-8,9-difluoro-6-oxo-1,4,5,6-tetrahydro-2H-pyrano[3,4-c]isoquinolin-1-yl)-N-methylacetamide;
(R)-2-amino-2-(3-chlorophenyl)-N-((R)-8,9-difluoro-6-oxo-1,4,5,6-tetrahydro-2H-pyrano[3,4-c]isoquinolin-1-yl)-N-methylacetamide;
2-amino-2-(3-chlorophenyl)-N-((S)-8,9-difluoro-6-oxo-1,4,5,6-tetrahydro-2H-pyrano[3,4-c]isoquinolin-1-yl)-N-methylacetamide;
2-amino-2-(3-chlorophenyl)-N-((R)-8,9-difluoro-6-oxo-1,4,5,6-tetrahydro-2H-pyrano[3,4-c]isoquinolin-1-yl)-N-methylacetamide;
(S)-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
(R)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
(S)-5-chloro-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
(R)-5-chloro-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
(S)-N-(8,9-difluoro-6-oxo-1,2,3,4,5,6-hexahydrobenzo[c][1,7]naphthyridin-1-yl)-4-(difluoromethyl)-N-methyl-1H-indole-2-carboxamide;
(R)-N-(8,9-difluoro-6-oxo-1,2,3,4,5,6-hexahydrobenzo[c][1,7]naphthyridin-1-yl)-4-(difluoromethyl)-N-methyl-1H-indole-2-carboxamide;
(S)-4-(difluoromethyl)-N-(8-fluoro-6-oxo-1,2,3,4,5,6-hexahydrobenzo[c][1,7]naphthyridin-1-yl)-N-methyl-1H-indole-2-carboxamide;
(R)-4-(difluoromethyl)-N-(8-fluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-1-hydroxy-N-methylcyclohexane-1-carboxamide;
(R)-N-(8,9-difluoro-6-oxo-1,4,5,6-tetrahydro-2H-pyrano[3,4-c]isoquinolin-1-yl)-1-hydroxy-N-methylcyclohexane-1-carboxamide;
(S)-N-(8,9-difluoro-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(S)-5-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-5-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-b]pyridine-2-carboxamide;
(R)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-b]pyridine-2-carboxamide;
(S)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
(R)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
(S)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
(R)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
(S)-6-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
(R)-6-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[3,2-b]pyridine-2-carboxamide;
(R)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[3,2-b]pyridine-2-carboxamide;
(S)-N-(8,9-difluoro-6-oxo-1,2,3,4,5,6-hexahydrobenzo[c][1,7]naphthyridin-1-yl)-6-(difluoromethyl)-5-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-6-(difluoromethyl)-5-fluoro-N-methyl-1H-indole-2-carboxamide;
(S)-N-(8,9-difluoro-6-oxo-1,2,3,4,5,6-hexahydrobenzo[c][1,7]naphthyridin-1-yl)-6-fluoro-N-methylindolizine-2-carboxamide;
(R)-N-(8,9-difluoro-6-oxo-1,2,3,4,5,6-hexahydrobenzo[c][1,7]naphthyridin-1-yl)-6-fluoro-N-methylindolizine-2-carboxamide;
(S)-N-(8,9-difluoro-6-oxo-1,4,5,6-tetrahydro-2H-pyrano[3,4-c]isoquinolin-1-yl)-6-fluoro-N-methylindolizine-2-carboxamide;
(R)-N-(8,9-difluoro-6-oxo-1,4,5,6-tetrahydro-2H-pyrano[3,4-c]isoquinolin-1-yl)-6-fluoro-N-methylindolizine-2-carboxamide;
(S)-N-(8,9-difluoro-6-oxo-1,4,5,6-tetrahydro-2H-pyrano[3,4-c]isoquinolin-1-yl)-N-methyl-2-(trifluoromethyl)-1H-imidazole-5-carboxamide;
(R)-N-(8,9-difluoro-6-oxo-1,4,5,6-tetrahydro-2H-pyrano[3,4-c]isoquinolin-1-yl)-N-methyl-2-(trifluoromethyl)-1H-imidazole-5-carboxamide;
(S)-N-(8,9-difluoro-6-oxo-1,4,5,6-tetrahydro-2H-pyrano[3,4-c]isoquinolin-1-yl)-5-fluoro-N-methyl-6-oxo-1,6-dihydropyridine-3-carboxamide;
(R)-N-(8,9-difluoro-6-oxo-1,4,5,6-tetrahydro-2H-pyrano[3,4-c]isoquinolin-1-yl)-5-fluoro-N-methyl-6-oxo-1,6-dihydropyridine-3-carboxamide;
(S)-N-(8,9-difluoro-6-oxo-1,4,5,6-tetrahydro-2H-pyrano[3,4-c]isoquinolin-1-yl)-N-methylindolizine-6-carboxamide;
(R)-N-(8,9-difluoro-6-oxo-1,4,5,6-tetrahydro-2H-pyrano[3,4-c]isoquinolin-1-yl)-N-methylindolizine-6-carboxamide;
(S)-N-(8,9-difluoro-6-oxo-1,4,5,6-tetrahydro-2H-pyrano[3,4-c]isoquinolin-1-yl)-N-methylindolizine-7-carboxamide;
(R)-N-(8,9-difluoro-6-oxo-1,4,5,6-tetrahydro-2H-pyrano[3,4-c]isoquinolin-1-yl)-N-methylindolizine-7-carboxamide;
(S)-N-(8,9-difluoro-6-oxo-1,4,5,6-tetrahydro-2H-pyrano[3,4-c]isoquinolin-1-yl)-2-hydroxy-N-methyl-2,2-diphenylacetamide;
(R)-N-(8,9-difluoro-6-oxo-1,4,5,6-tetrahydro-2H-pyrano[3,4-c]isoquinolin-1-yl)-2-hydroxy-N-methyl-2,2-diphenylacetamide;
(S)-N-(8,9-difluoro-6-oxo-1,4,5,6-tetrahydro-2H-pyrano[3,4-c]isoquinolin-1-yl)-4-(difluoromethyl)-6-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4-(difluoromethyl)-6-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-7-fluoro-N-methylindolizine-2-carboxamide;
(R)-N-(8,9-difluoro-6-oxo-1,4,5,6-tetrahydro-2H-pyrano[3,4-c]isoquinolin-1-yl)-7-fluoro-N-methylindolizine-2-carboxamide;
(S)-N-(8,9-difluoro-6-oxo-1,4,5,6-tetrahydro-2H-pyrano[3,4-c]isoquinolin-1-yl)-5-fluoro-N-methyl-1H-pyrrolo[3,2-b]pyridine-2-carboxamide;
(R)-N-(8,9-difluoro-6-oxo-1,4,5,6-tetrahydro-2H-pyrano[3,4-c]isoquinolin-1-yl)-5-fluoro-N-methyl-1H-pyrrolo[3,2-b]pyridine-2-carboxamide;
(S)-N-(8,9-difluoro-6-oxo-1,4,5,6-tetrahydro-2H-pyrano[3,4-c]isoquinolin-1-yl)-5,6-difluoro-N-methyl-2,3-dihydro-1H-indene-2-carboxamide;
(R)-N-(8,9-difluoro-6-oxo-1,4,5,6-tetrahydro-2H-pyrano[3,4-c]isoquinolin-1-yl)-5,6-difluoro-N-methyl-2,3-dihydro-1H-indene-2-carboxamide;
(S)-N-(8,9-difluoro-6-oxo-1,4,5,6-tetrahydro-2H-pyrano[3,4-c]isoquinolin-1-yl)-1-(4-fluorophenyl)-N-methyl-1H-pyrazole-4-carboxamide;
(R)-N-(8,9-difluoro-6-oxo-1,4,5,6-tetrahydro-2H-pyrano[3,4-c]isoquinolin-1-yl)-1-(4-fluorophenyl)-N-methyl-1H-pyrazole-4-carboxamide;
(S)-N-(8,9-difluoro-6-oxo-1,4,5,6-tetrahydro-2H-pyrano[3,4-c]isoquinolin-1-yl)-3-(4-fluorophenyl)-N-methylisoxazole-5-carboxamide;
(R)-N-(8,9-difluoro-6-oxo-1,4,5,6-tetrahydro-2H-pyrano[3,4-c]isoquinolin-1-yl)-3-(4-fluorophenyl)-N-methylisoxazole-5-carboxamide;
(S)-N-(8,9-difluoro-6-oxo-1,4,5,6-tetrahydro-2H-pyrano[3,4-c]isoquinolin-1-yl)-4-(3-fluorophenoxy)-N-methylbenzamide;
(R)-N-(8,9-difluoro-6-oxo-1,4,5,6-tetrahydro-2H-pyrano[3,4-c]isoquinolin-1-yl)-4-(3-fluorophenoxy)-N-methylbenzamide;
(S)-4-(difluoromethyl)-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-(difluoromethyl)-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-5-methyl-6-oxo-1,4,5,6-tetrahydro-2H-pyrano[3,4-c]isoquinolin-1-yl)-5,6-difluoro-N-methyl-1H-indole-2-carboxamide;
(R)-N-(8,9-difluoro-5-methyl-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-methoxy-1,4-dihydro-2H-pyrano[3,4-c]isoquinolin-1-yl)-5,6-difluoro-N-methyl-1H-indole-2-carboxamide;
(R)-N-(8,9-difluoro-6-methoxy-1,4-dihydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-5,6,7,8-tetrahydroindolizine-2-carboxamide;
(R)-N-(8,9-difluoro-6-oxo-1,4,5,6-tetrahydro-2H-pyrano[3,4-c]isoquinolin-1-yl)-N-methyl-5,6,7,8-tetrahydroindolizine-2-carboxamide;
(S)-4-bromo-N-(8,9-difluoro-6-oxo-1,4,5,6-tetrahydro-2H-pyrano[3,4-c]isoquinolin-1-yl)-2,3-difluoro-N-methylbenzamide;
(R)-4-bromo-N-(8,9-difluoro-6-oxo-1,4,5,6-tetrahydro-2H-pyrano[3,4-c]isoquinolin-1-yl)-2,3-difluoro-N-methylbenzamide;
(S)-4-bromo-N-(8,9-difluoro-6-oxo-1,4,5,6-tetrahydro-2H-pyrano[3,4-c]isoquinolin-1-yl)-2,5-difluoro-N-methylbenzamide;
(R)-4-bromo-N-(8,9-difluoro-6-oxo-1,4,5,6-tetrahydro-2H-pyrano[3,4-c]isoquinolin-1-yl)-2,5-difluoro-N-methylbenzamide;
(S)-4-bromo-N-(8,9-difluoro-6-oxo-1,4,5,6-tetrahydro-2H-pyrano[3,4-c]isoquinolin-1-yl)-2,6-difluoro-N-methylbenzamide;
(R)-4-bromo-N-(8,9-difluoro-6-oxo-1,4,5,6-tetrahydro-2H-pyrano[3,4-c]isoquinolin-1-yl)-2,6-difluoro-N-methylbenzamide;
(S)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-2-carboxamide;
(R)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-2-carboxamide;
(S)-N-(8,9-difluoro-6-oxo-1,4,5,6-tetrahydro-2H-pyrano[3,4-c]isoquinolin-1-yl)-N-methylindolizine-3-carboxamide;
(R)-N-(8,9-difluoro-6-oxo-1,4,5,6-tetrahydro-2H-pyrano[3,4-c]isoquinolin-1-yl)-N-methylindolizine-3-carboxamide;
(S)-N-(8,9-difluoro-6-oxo-1,4,5,6-tetrahydro-2H-pyrano[3,4-c]isoquinolin-1-yl)-4-(4-fluorophenoxy)-N-methylbenzamide;
(R)-N-(8,9-difluoro-6-oxo-1,4,5,6-tetrahydro-2H-pyrano[3,4-c]isoquinolin-1-yl)-4-(4-fluorophenoxy)-N-methylbenzamide;
(S)-N-(8,9-difluoro-6-oxo-1,4,5,6-tetrahydro-2H-pyrano[3,4-c]isoquinolin-1-yl)-4′-fluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-4′-fluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-c]pyridine-2-carboxamide;
(R)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-c]pyridine-2-carboxamide;
(S)-2-(3-bromophenyl)-N-(8,9-difluoro-6-oxo-1,4,5,6-tetrahydro-2H-pyrano[3,4-c]isoquinolin-1-yl)-2,2-difluoro-N-methylacetamide;
(R)-2-(3-bromophenyl)-N-(8,9-difluoro-6-oxo-1,4,5,6-tetrahydro-2H-pyrano[3,4-c]isoquinolin-1-yl)-2,2-difluoro-N-methylacetamide;
(S)-2-(4-bromophenyl)-N-(8,9-difluoro-6-oxo-1,4,5,6-tetrahydro-2H-pyrano[3,4-c]isoquinolin-1-yl)-2,2-difluoro-N-methylacetamide;
(R)-2-(4-bromophenyl)-N-(8,9-difluoro-6-oxo-1,4,5,6-tetrahydro-2H-pyrano[3,4-c]isoquinolin-1-yl)-2,2-difluoro-N-methylacetamide;
(S)-N-(8,9-difluoro-6-oxo-1,4,5,6-tetrahydro-2H-pyrano[3,4-c]isoquinolin-1-yl)-4-(difluoromethyl)-5-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4-(difluoromethyl)-5-fluoro-N-methyl-1H-indole-2-carboxamide;
(S)-4-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,2,3,4,5,6-hexahydrobenzo[c][1,7]naphthyridin-1-yl)-4-(difluoromethyl)-6-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-4-(difluoromethyl)-6-fluoro-N-methyl-1H-indole-2-carboxamide;
(S)-4-(difluoromethyl)-6-fluoro-N-(8-fluoro-6-oxo-1,2,3,4,5,6-hexahydrobenzo[c][1,7]naphthyridin-1-yl)-N-methyl-1H-indole-2-carboxamide;
(R)-4-(difluoromethyl)-6-fluoro-N-(8-fluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methylindolizine-1-carboxamide;
(R)-N-(8,9-difluoro-6-oxo-1,4,5,6-tetrahydro-2H-pyrano[3,4-c]isoquinolin-1-yl)-N-methylindolizine-1-carboxamide;
(S)-N-(8,9-difluoro-6-oxo-1,4,5,6-tetrahydro-2H-pyrano[3,4-c]isoquinolin-1-yl)-2,4′-difluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-2,4′-difluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-5-(4-fluorophenyl)-N-methylisoxazole-3-carboxamide;
(R)-N-(8,9-difluoro-6-oxo-1,4,5,6-tetrahydro-2H-pyrano[3,4-c]isoquinolin-1-yl)-5-(4-fluorophenyl)-N-methylisoxazole-3-carboxamide;
(S)-N-(8,9-difluoro-6-oxo-1,2,3,4,5,6-hexahydrobenzo[c][1,7]naphthyridin-1-yl)-4-(difluoromethyl)-5-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-4-(difluoromethyl)-5-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-5-(difluoromethyl)-N-methylindolizine-2-carboxamide;
(R)-N-(8,9-difluoro-6-oxo-1,4,5,6-tetrahydro-2H-pyrano[3,4-c]isoquinolin-1-yl)-5-(difluoromethyl)-N-methylindolizine-2-carboxamide;
(S)-5-bromo-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
(R)-5-bromo-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
N-((S)-8,9-difluoro-6-oxo-1,4,5,6-tetrahydro-2H-pyrano[3,4-c]isoquinolin-1-yl)-N-methylbicyclo[4.2.0]octa-1(6),2,4-triene-7-carboxamide;
N-((R)-8,9-difluoro-6-oxo-1,4,5,6-tetrahydro-2H-pyrano[3,4-c]isoquinolin-1-yl)-N-methylbicyclo[4.2.0]octa-1(6),2,4-triene-7-carboxamide;
(S)-4-(difluoromethyl)-5-fluoro-N-(8-fluoro-6-oxo-1,2,3,4,5,6-hexahydrobenzo[c][1,7]naphthyridin-1-yl)-N-methyl-1H-indole-2-carboxamide;
(R)-4-(difluoromethyl)-5-fluoro-N-(8-fluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-2-hydroxy-N-methyl-2,3-dihydro-1H-indene-2-carboxamide;
(R)-N-(8,9-difluoro-6-oxo-1,4,5,6-tetrahydro-2H-pyrano[3,4-c]isoquinolin-1-yl)-2-hydroxy-N-methyl-2,3-dihydro-1H-indene-2-carboxamide;
(S)-N-(8,9-difluoro-6-oxo-1,4,5,6-tetrahydro-2H-pyrano[3,4-c]isoquinolin-1-yl)-5,6-difluoro-2-hydroxy-N-methyl-2,3-dihydro-1H-indene-2-carboxamide;
(R)-N-(8,9-difluoro-6-oxo-1,4,5,6-tetrahydro-2H-pyrano[3,4-c]isoquinolin-1-yl)-5,6-difluoro-2-hydroxy-N-methyl-2,3-dihydro-1H-indene-2-carboxamide;
(S)-N-(8,9-difluoro-6-oxo-1,4,5,6-tetrahydro-2H-pyrano[3,4-c]isoquinolin-1-yl)-N,2-dimethylindolizine-6-carboxamide;
(R)-N-(8,9-difluoro-6-oxo-1,4,5,6-tetrahydro-2H-pyrano[3,4-c]isoquinolin-1-yl)-N,2-dimethylindolizine-6-carboxamide;
(S)-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
N-((S)-8,9-difluoro-6-oxo-1,4,5,6-tetrahydro-2H-pyrano[3,4-c]isoquinolin-1-yl)-4-fluoro-N-methylbicyclo[4.2.0]octa-1(6),2,4-triene-7-carboxamide;
N-((R)-8,9-difluoro-6-oxo-1,4,5,6-tetrahydro-2H-pyrano[3,4-c]isoquinolin-1-yl)-4-fluoro-N-methylbicyclo[4.2.0]octa-1(6),2,4-triene-7-carboxamide;
(S)-N-(6-(2-aminoethoxy)-8,9-difluoro-1,4-dihydro-2H-pyrano[3,4-c]isoquinolin-1-yl)-5,6-difluoro-N-methyl-1H-indole-2-carboxamide;
(R)-N-(6-(2-aminoethoxy)-8,9-difluoro-1,4-dihydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-1-(thiophen-3-yl)azetidine-3-carboxamide;
(R)-N-(8,9-difluoro-6-oxo-1,4,5,6-tetrahydro-2H-pyrano[3,4-c]isoquinolin-1-yl)-N-methyl-1-(thiophen-3-yl)azetidine-3-carboxamide;
(S)-N-(8,9-difluoro-6-oxo-1,4,5,6-tetrahydro-2H-pyrano[3,4-c]isoquinolin-1-yl)-3′-fluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-3′-fluoro-N-methyl-[1,1′-biphenyl]-4-carboxamide;
(S)-1-(4-bromothiophen-3-yl)-N-(8,9-difluoro-6-oxo-1,4,5,6-tetrahydro-2H-pyrano[3,4-c]isoquinolin-1-yl)-N-methylazetidine-3-carboxamide;
(R)-1-(4-bromothiophen-3-yl)-N-(8,9-difluoro-6-oxo-1,4,5,6-tetrahydro-2H-pyrano[3,4-c]isoquinolin-1-yl)-N-methylazetidine-3-carboxamide;
(S)-N-(8,9-difluoro-6-oxo-1,4,5,6-tetrahydro-2H-pyrano[3,4-c]isoquinolin-1-yl)-2′-fluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-2′-fluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-3′,5′-difluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-3′,5′-difluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-2,3′,5′-trifluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-2,3′,5′-trifluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-N-methyl-[1,1′-biphenyl]-3-carboxamide;
(R)-N-(8,9-difluoro-6-oxo-1,4,5,6-tetrahydro-2H-pyrano[3,4-c]isoquinolin-1-yl)-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-4′-fluoro-N-methyl-[1,1′-biphenyl]-3-carboxamide;
(R)-N-(8,9-difluoro-6-oxo-1,4,5,6-tetrahydro-2H-pyrano[3,4-c]isoquinolin-1-yl)-4′-fluoro-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-3′-fluoro-N-methyl-[1,1′-biphenyl]-3-carboxamide;
(R)-N-(8,9-difluoro-6-oxo-1,4,5,6-tetrahydro-2H-pyrano[3,4-c]isoquinolin-1-yl)-3′-fluoro-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-3′,5-difluoro-N-methyl-[1,1′-biphenyl]-3-carboxamide;
(R)-N-(8,9-difluoro-6-oxo-1,4,5,6-tetrahydro-2H-pyrano[3,4-c]isoquinolin-1-yl)-3′,5-difluoro-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-N-methyl-3-phenoxybenzamide;
(R)-N-(8,9-difluoro-6-oxo-1,4,5,6-tetrahydro-2H-pyrano[3,4-c]isoquinolin-1-yl)-N-methyl-3-phenoxybenzamide;
(S)-N-(8,9-difluoro-6-oxo-1,4,5,6-tetrahydro-2H-pyrano[3,4-c]isoquinolin-1-yl)-3-(4-fluorophenoxy)-N-methylbenzamide;
(R)-N-(8,9-difluoro-6-oxo-1,4,5,6-tetrahydro-2H-pyrano[3,4-c]isoquinolin-1-yl)-3-(4-fluorophenoxy)-N-methylbenzamide;
(S)-N-(8,9-difluoro-6-oxo-1,4,5,6-tetrahydro-2H-pyrano[3,4-c]isoquinolin-1-yl)-N-methyl-6H-thieno[2,3-b]pyrrole-5-carboxamide;
(R)-N-(8,9-difluoro-6-oxo-1,4,5,6-tetrahydro-2H-pyrano[3,4-c]isoquinolin-1-yl)-N-methyl-6H-thieno[2,3-b]pyrrole-5-carboxamide;
(S)-N-(8,9-difluoro-6-oxo-1,4,5,6-tetrahydro-2H-pyrano[3,4-c]isoquinolin-1-yl)-N-methyl-4-(methylsulfonyl)benzamide;
(R)-N-(8,9-difluoro-6-oxo-1,4,5,6-tetrahydro-2H-pyrano[3,4-c]isoquinolin-1-yl)-N-methyl-4-(methylsulfonyl)benzamide;
(S)-N-(8,9-difluoro-6-oxo-1,4,5,6-tetrahydro-2H-pyrano[3,4-c]isoquinolin-1-yl)-N-methyl-3-(N-methylsulfamoyl)benzamide;
(R)-N-(8,9-difluoro-6-oxo-1,4,5,6-tetrahydro-2H-pyrano[3,4-c]isoquinolin-1-yl)-N-methyl-3-(N-methylsulfamoyl)benzamide;
(S)-N-(8,9-difluoro-6-oxo-1,4,5,6-tetrahydro-2H-pyrano[3,4-c]isoquinolin-1-yl)-N-methyl-3-(methylsulfonamido)benzamide;
(R)-N-(8,9-difluoro-6-oxo-1,4,5,6-tetrahydro-2H-pyrano[3,4-c]isoquinolin-1-yl)-N-methyl-3-(methylsulfonamido)benzamide;
(S)-N-(8,9-difluoro-6-oxo-1,4,5,6-tetrahydro-2H-pyrano[3,4-c]isoquinolin-1-yl)-N-methyl-3-(methylsulfonyl)benzamide;
(R)-N-(8,9-difluoro-6-oxo-1,4,5,6-tetrahydro-2H-pyrano[3,4-c]isoquinolin-1-yl)-N-methyl-3-(methylsulfonyl)benzamide;
(S)-N-(8,9-difluoro-6-oxo-1,4,5,6-tetrahydro-2H-pyrano[3,4-c]isoquinolin-1-yl)-N-methylimidazo[1,5-a]pyridine-6-carboxamide;
(R)-N-(8,9-difluoro-6-oxo-1,4,5,6-tetrahydro-2H-pyrano[3,4-c]isoquinolin-1-yl)-N-methylimidazo[1,5-a]pyridine-6-carboxamide;
(1S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1S,4S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1S,4R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1R,4R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1R,4S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(S)-N-(8,9-difluoro-6-oxo-1,2,3,4,5,6-hexahydrobenzo[c][1,7]naphthyridin-1-yl)-3′-fluoro-N-methyl-[1,1′-biphenyl]-4-carboxamide;
(R)-N-(8,9-difluoro-6-oxo-1,2,3,4,5,6-hexahydrobenzo[c][1,7]naphthyridin-1-yl)-3′-fluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-3-fluoro-N-methyl-5-phenoxybenzamide;
(R)-N-(8,9-difluoro-6-oxo-1,4,5,6-tetrahydro-2H-pyrano[3,4-c]isoquinolin-1-yl)-3-fluoro-N-methyl-5-phenoxybenzamide;
(S)-N-(8,9-difluoro-6-oxo-1,2,3,4,5,6-hexahydrobenzo[c][1,7]naphthyridin-1-yl)-3′-fluoro-N-methyl-[1,1′-biphenyl]-3-carboxamide;
(R)-N-(8,9-difluoro-6-oxo-1,2,3,4,5,6-hexahydrobenzo[c][1,7]naphthyridin-1-yl)-3′-fluoro-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-N-methylimidazo[1,2-a]pyridine-6-carboxamide;
(R)-N-(8,9-difluoro-6-oxo-1,4,5,6-tetrahydro-2H-pyrano[3,4-c]isoquinolin-1-yl)-N-methylimidazo[1,2-a]pyridine-6-carboxamide;
(S)-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(R)-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(S)-6-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(R)-6-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(S)-N-(8,9-difluoro-6-oxo-1,4,5,6-tetrahydro-2H-pyrano[3,4-c]isoquinolin-1-yl)-5-fluoro-N-methyl-1H-pyrrolo[2,3-c]pyridine-2-carboxamide;
(R)-N-(8,9-difluoro-6-oxo-1,4,5,6-tetrahydro-2H-pyrano[3,4-c]isoquinolin-1-yl)-5-fluoro-N-methyl-1H-pyrrolo[2,3-c]pyridine-2-carboxamide;
(S)-N-(8,9-difluoro-6-oxo-1,4,5,6-tetrahydro-2H-pyrano[3,4-c]isoquinolin-1-yl)-N-methyl-2-(trifluoromethyl)indolizine-6-carboxamide;
(R)-N-(8,9-difluoro-6-oxo-1,4,5,6-tetrahydro-2H-pyrano[3,4-c]isoquinolin-1-yl)-N-methyl-2-(trifluoromethyl)indolizine-6-carboxamide;
(S)-4-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-4-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-N-(8,9-difluoro-6-oxo-1,4,5,6-tetrahydro-2H-pyrano[3,4-c]isoquinolin-1-yl)-2-(difluoromethyl)-N-methylisonicotinamide;
(R)-N-(8,9-difluoro-6-oxo-1,4,5,6-tetrahydro-2H-pyrano[3,4-c]isoquinolin-1-yl)-2-(difluoromethyl)-N-methylisonicotinamide;
(S)-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
(R)-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
(S)-N-(8,9-difluoro-5-(2-hydroxyethyl)-6-oxo-1,4,5,6-tetrahydro-2H-pyrano[3,4-c]isoquinolin-1-yl)-5,6-difluoro-N-methyl-1H-indole-2-carboxamide;
(R)-N-(8,9-difluoro-5-(2-hydroxyethyl)-6-oxo-1,4,5,6-tetrahydro-2H-pyrano[3,4-c]isoquinolin-1-yl)-5,6-difluoro-N-methyl-1H-indole-2-carboxamide;
(S)-N-(5-(2-aminoethyl)-8,9-difluoro-6-oxo-1,4,5,6-tetrahydro-2H-pyrano[3,4-c]isoquinolin-1-yl)-5,6-difluoro-N-methyl-1H-indole-2-carboxamide;
(R)-N-(5-(2-aminoethyl)-8,9-difluoro-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-(2-hydroxyethoxy)-1,4-dihydro-2H-pyrano[3,4-c]isoquinolin-1-yl)-5,6-difluoro-N-methyl-1H-indole-2-carboxamide;
(R)-N-(8,9-difluoro-6-(2-hydroxyethoxy)-1,4-dihydro-2H-pyrano[3,4-c]isoquinolin-1-yl)-5,6-difluoro-N-methyl-1H-indole-2-carboxamide;
(S)-N-(8,9-difluoro-6-oxo-1,2,3,4,5,6-hexahydrobenzo[c][1,7]naphthyridin-1-yl)-N-methyl-3-(N-methylsulfamoyl)benzamide;
(R)-N-(8,9-difluoro-6-oxo-1,2,3,4,5,6-hexahydrobenzo[c][1,7]naphthyridin-1-yl)-N-methyl-3-(N-methylsulfamoyl)benzamide;
N-((1S)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1S,4R)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1S,4S)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1R)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1R,4R)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1R,4S)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-4,6-dioxo-1,4,5,6-tetrahydro-2H-pyrano[3,4-c]isoquinolin-1-yl)-5,6-difluoro-N-methyl-1H-indole-2-carboxamide;
(R)-N-(8,9-difluoro-4,6-dioxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-3-(3-fluorophenoxy)-N-methylbenzamide;
(R)-N-(8,9-difluoro-6-oxo-1,4,5,6-tetrahydro-2H-pyrano[3,4-c]isoquinolin-1-yl)-3-(3-fluorophenoxy)-N-methylbenzamide;
(S)-N-(8,9-difluoro-6-oxo-1,4,5,6-tetrahydro-2H-pyrano[3,4-c]isoquinolin-1-yl)-5-methoxy-N-methyl-1H-indole-2-carboxamide;
(R)-N-(8,9-difluoro-6-oxo-1,4,5,6-tetrahydro-2H-pyrano[3,4-c]isoquinolin-1-yl)-5-methoxy-N-methyl-1H-indole-2-carboxamide;
(S)-5-chloro-N-(8,9-difluoro-6-oxo-1,4,5,6-tetrahydro-2H-pyrano[3,4-c]isoquinolin-1-yl)-N-methyl-1H-indole-2-carboxamide;
(R)-5-chloro-N-(8,9-difluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N,5-dimethyl-1H-indole-2-carboxamide;
(R)-N-(8,9-difluoro-6-oxo-1,4,5,6-tetrahydro-2H-pyrano[3,4-c]isoquinolin-1-yl)-N,5-dimethyl-1H-indole-2-carboxamide;
(S)-N-(8,9-difluoro-6-oxo-1,4,5,6-tetrahydro-2H-pyrano[3,4-c]isoquinolin-1-yl)-N-methyl-5-(trifluoromethoxy)-1H-indole-2-carboxamide;
(R)-N-(8,9-difluoro-6-oxo-1,4,5,6-tetrahydro-2H-pyrano[3,4-c]isoquinolin-1-yl)-N-methyl-5-(trifluoromethoxy)-1H-indole-2-carboxamide;
(S)-N-(8,9-difluoro-6-oxo-1,4,5,6-tetrahydro-2H-pyrano[3,4-c]isoquinolin-1-yl)-5-ethyl-N-methyl-1H-indole-2-carboxamide;
(R)-N-(8,9-difluoro-6-oxo-1,4,5,6-tetrahydro-2H-pyrano[3,4-c]isoquinolin-1-yl)-5-ethyl-N-methyl-1H-indole-2-carboxamide;
(S)-N-(8,9-difluoro-6-oxo-1,4,5,6-tetrahydro-2H-pyrano[3,4-c]isoquinolin-1-yl)-4-methoxy-N-methyl-1H-indole-2-carboxamide;
(R)-N-(8,9-difluoro-6-oxo-1,4,5,6-tetrahydro-2H-pyrano[3,4-c]isoquinolin-1-yl)-4-methoxy-N-methyl-1H-indole-2-carboxamide;
(S)-N-(8,9-difluoro-6-oxo-1,4,5,6-tetrahydro-2H-pyrano[3,4-c]isoquinolin-1-yl)-N,4-dimethyl-1H-indole-2-carboxamide;
(R)-N-(8,9-difluoro-6-oxo-1,4,5,6-tetrahydro-2H-pyrano[3,4-c]isoquinolin-1-yl)-N,4-dimethyl-1H-indole-2-carboxamide;
(1S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1S,4S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1S,4R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1R,4S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1R,4R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(S)-2-chloro-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
(R)-2-chloro-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
(S)-N-(8,9-difluoro-6-(methylamino)-1,4-dihydro-2H-pyrano[3,4-c]isoquinolin-1-yl)-5,6-difluoro-N-methyl-1H-indole-2-carboxamide;
(R)-N-(8,9-difluoro-6-(methylamino)-1,4-dihydro-2H-pyrano[3,4-c]isoquinolin-1-yl)-5,6-difluoro-N-methyl-1H-indole-2-carboxamide;
(S)-N-(8,9-difluoro-6-((2-hydroxyethyl)amino)-1,4-dihydro-2H-pyrano[3,4-c]isoquinolin-1-yl)-5,6-difluoro-N-methyl-1H-indole-2-carboxamide;
(R)-N-(8,9-difluoro-6-((2-hydroxyethyl)amino)-1,4-dihydro-2H-pyrano[3,4-c]isoquinolin-1-yl)-5,6-difluoro-N-methyl-1H-indole-2-carboxamide;
(S)-N-(8,9-difluoro-6-oxo-1,2,3,4,5,6-hexahydrobenzo[c][1,7]naphthyridin-1-yl)-3-(3-fluorophenoxy)-N-methylbenzamide;
(R)-N-(8,9-difluoro-6-oxo-1,2,3,4,5,6-hexahydrobenzo[c][1,7]naphthyridin-1-yl)-3-(3-fluorophenoxy)-N-methylbenzamide;
(S)-4-cyano-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(R)-4-cyano-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-4-ethyl-6-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4-ethyl-6-fluoro-N-methyl-1H-indole-2-carboxamide;
(S)-4-chloro-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(R)-4-chloro-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-6-fluoro-N,4-dimethyl-1H-indole-2-carboxamide;
(R)-N-(8,9-difluoro-6-oxo-1,4,5,6-tetrahydro-2H-pyrano[3,4-c]isoquinolin-1-yl)-6-fluoro-N,4-dimethyl-1H-indole-2-carboxamide;
(S)-5-cyano-N-(8,9-difluoro-6-oxo-1,4,5,6-tetrahydro-2H-pyrano[3,4-c]isoquinolin-1-yl)-N-methyl-1H-indole-2-carboxamide;
(R)-5-cyano-N-(8,9-difluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-5-(methylsulfonyl)-1H-indole-2-carboxamide;
(R)-N-(8,9-difluoro-6-oxo-1,4,5,6-tetrahydro-2H-pyrano[3,4-c]isoquinolin-1-yl)-N-methyl-5-(methylsulfonyl)-1H-indole-2-carboxamide;
(S)-4-chloro-N-(8,9-difluoro-6-oxo-1,4,5,6-tetrahydro-2H-pyrano[3,4-c]isoquinolin-1-yl)-N-methyl-1H-indole-2-carboxamide;
(R)-4-chloro-N-(8,9-difluoro-6-oxo-1,4,5,6-tetrahydro-2H-pyrano[3,4-c]isoquinolin-1-yl)-N-methyl-1H-indole-2-carboxamide;
(S)-6-bromo-N-(8,9-difluoro-6-oxo-1,4,5,6-tetrahydro-2H-pyrano[3,4-c]isoquinolin-1-yl)-N-methylindolizine-2-carboxamide;
(R)-6-bromo-N-(8,9-difluoro-6-oxo-1,4,5,6-tetrahydro-2H-pyrano[3,4-c]isoquinolin-1-yl)-N-methylindolizine-2-carboxamide;
(S)-2-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-2-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-3-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-3-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-N-(6-((2-aminoethyl)amino)-8,9-difluoro-1,4-dihydro-2H-pyrano[3,4-c]isoquinolin-1-yl)-5,6-difluoro-N-methyl-1H-indole-2-carboxamide;
(R)-N-(6-((2-aminoethyl)amino)-8,9-difluoro-1,4-dihydro-2H-pyrano[3,4-c]isoquinolin-1-yl)-5,6-difluoro-N-methyl-1H-indole-2-carboxamide;
N-((S)-8,9-difluoro-6-oxo-1,4,5,6-tetrahydro-2H-pyrano[3,4-c]isoquinolin-1-yl)-6-fluoro-4-(1-hydroxyethyl)-N-methyl-1H-indole-2-carboxamide;
N-((R)-8,9-difluoro-6-oxo-1,4,5,6-tetrahydro-2H-pyrano[3,4-c]isoquinolin-1-yl)-6-fluoro-4-(1-hydroxyethyl)-N-methyl-1H-indole-2-carboxamide;
N-((S)-8,9-difluoro-6-oxo-1,4,5,6-tetrahydro-2H-pyrano[3,4-c]isoquinolin-1-yl)-6-fluoro-4-((S)-1-hydroxyethyl)-N-methyl-1H-indole-2-carboxamide;
N-((R)-8,9-difluoro-6-oxo-1,4,5,6-tetrahydro-2H-pyrano[3,4-c]isoquinolin-1-yl)-6-fluoro-4-((S)-1-hydroxyethyl)-N-methyl-1H-indole-2-carboxamide;
N-((S)-8,9-difluoro-6-oxo-1,4,5,6-tetrahydro-2H-pyrano[3,4-c]isoquinolin-1-yl)-6-fluoro-4-((R)-1-hydroxyethyl)-N-methyl-1H-indole-2-carboxamide;
N-((S)-8,9-difluoro-6-oxo-1,4,5,6-tetrahydro-2H-pyrano[3,4-c]isoquinolin-1-yl)-6-fluoro-4-((S)-1-hydroxyethyl)-N-methyl-1H-indole-2-carboxamide;
(S)-N-(8,9-difluoro-6-oxo-1,4,5,6-tetrahydro-2H-pyrano[3,4-c]isoquinolin-1-yl)-6-fluoro-4-(hydroxymethyl)-N-methyl-1H-indole-2-carboxamide;
(R)-N-(8,9-difluoro-6-oxo-1,4,5,6-tetrahydro-2H-pyrano[3,4-c]isoquinolin-1-yl)-6-fluoro-4-(hydroxymethyl)-N-methyl-1H-indole-2-carboxamide;
(S)-7-bromo-N-(8,9-difluoro-6-oxo-1,4,5,6-tetrahydro-2H-pyrano[3,4-c]isoquinolin-1-yl)-N-methylfuro[3,2-c]pyridine-2-carboxamide;
(R)-7-bromo-N-(8,9-difluoro-6-oxo-1,4,5,6-tetrahydro-2H-pyrano[3,4-c]isoquinolin-1-yl)-N-methylfuro[3,2-c]pyridine-2-carboxamide;
(S)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-6-carboxamide;
(R)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-6-carboxamide;
(S)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-6-carboxamide;
(R)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-6-carboxamide;
(S)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
(R)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
(S)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-5-carboxamide;
(R)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-5-carboxamide;
(S)-3,4-dichloro-N-(8,9-difluoro-6-oxo-1,4,5,6-tetrahydro-2H-pyrano[3,4-c]isoquinolin-1-yl)-N-methylbenzamide;
(R)-3,4-dichloro-N-(8,9-difluoro-6-oxo-1,4,5,6-tetrahydro-2H-pyrano[3,4-c]isoquinolin-1-yl)-N-methylbenzamide;
(S)-N-(8,9-difluoro-6-oxo-1,4,5,6-tetrahydro-2H-pyrano[3,4-c]isoquinolin-1-yl)-6-fluoro-N-methyl-4-(methylsulfonamido)-1H-indole-2-carboxamide;
(R)-N-(8,9-difluoro-6-oxo-1,4,5,6-tetrahydro-2H-pyrano[3,4-c]isoquinolin-1-yl)-6-fluoro-N-methyl-4-(methylsulfonamido)-1H-indole-2-carboxamide;
(S)-N-(8,9-difluoro-6-oxo-1,4,5,6-tetrahydro-2H-pyrano[3,4-c]isoquinolin-1-yl)-N-methyl-4-(trifluoromethoxy)-1H-indole-2-carboxamide;
(R)-N-(8,9-difluoro-6-oxo-1,4,5,6-tetrahydro-2H-pyrano[3,4-c]isoquinolin-1-yl)-N-methyl-4-(trifluoromethoxy)-1H-indole-2-carboxamide;
(S)-N-(8,9-difluoro-6-oxo-1,4,5,6-tetrahydro-2H-pyrano[3,4-c]isoquinolin-1-yl)-6-methoxy-N-methyl-1H-indole-2-carboxamide;
(R)-N-(8,9-difluoro-6-oxo-1,4,5,6-tetrahydro-2H-pyrano[3,4-c]isoquinolin-1-yl)-6-methoxy-N-methyl-1H-indole-2-carboxamide;
(S)-5-bromo-N-(8,9-difluoro-6-oxo-1,4,5,6-tetrahydro-2H-pyrano[3,4-c]isoquinolin-1-yl)-N-methyl-6-(trifluoromethyl)nicotinamide;
(R)-5-bromo-N-(8,9-difluoro-6-oxo-1,4,5,6-tetrahydro-2H-pyrano[3,4-c]isoquinolin-1-yl)-N-methyl-6-(trifluoromethyl)nicotinamide;
(S)-3-chloro-N-(8,9-difluoro-6-oxo-1,4,5,6-tetrahydro-2H-pyrano[3,4-c]isoquinolin-1-yl)-N-methylindolizine-6-carboxamide;
(R)-3-chloro-N-(8,9-difluoro-6-oxo-1,4,5,6-tetrahydro-2H-pyrano[3,4-c]isoquinolin-1-yl)-N-methylindolizine-6-carboxamide;
(S)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-6-carboxamide;
(R)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-6-carboxamide;
(S)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-5-carboxamide;
(R)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-5-carboxamide;
(S)-N-(8,9-difluoro-6-oxo-1,4,5,6-tetrahydro-2H-pyrano[3,4-c]isoquinolin-1-yl)-3-methoxy-N-methyl-1H-indazole-5-carboxamide;
(R)-N-(8,9-difluoro-6-oxo-1,4,5,6-tetrahydro-2H-pyrano[3,4-c]isoquinolin-1-yl)-3-methoxy-N-methyl-1H-indazole-5-carboxamide;
(S)-1-(5-chlorothiophen-3-yl)-N-(8,9-difluoro-6-oxo-1,4,5,6-tetrahydro-2H-pyrano[3,4-c]isoquinolin-1-yl)-N-methylazetidine-3-carboxamide;
(R)-1-(5-chlorothiophen-3-yl)-N-(8,9-difluoro-6-oxo-1,4,5,6-tetrahydro-2H-pyrano[3,4-c]isoquinolin-1-yl)-N-methylazetidine-3-carboxamide;
(S)-7-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-7-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-4-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(R)-4-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(S)-3-chloro-N-(8,9-difluoro-6-oxo-1,4,5,6-tetrahydro-2H-pyrano[3,4-c]isoquinolin-1-yl)-N-methylindolizine-7-carboxamide;
(R)-3-chloro-N-(8,9-difluoro-6-oxo-1,4,5,6-tetrahydro-2H-pyrano[3,4-c]isoquinolin-1-yl)-N-methylindolizine-7-carboxamide;
(S)-N-(8,9-difluoro-6-oxo-1,4,5,6-tetrahydro-2H-pyrano[3,4-c]isoquinolin-1-yl)-7-(difluoromethyl)-N-methylindolizine-2-carboxamide;
(R)-N-(8,9-difluoro-6-oxo-1,4,5,6-tetrahydro-2H-pyrano[3,4-c]isoquinolin-1-yl)-7-(difluoromethyl)-N-methylindolizine-2-carboxamide;
(S)-N-(8,9-difluoro-6-oxo-1,4,5,6-tetrahydro-2H-pyrano[3,4-c]isoquinolin-1-yl)-6-(difluoromethyl)-N-methylindolizine-2-carboxamide;
(R)-N-(8,9-difluoro-6-oxo-1,4,5,6-tetrahydro-2H-pyrano[3,4-c]isoquinolin-1-yl)-6-(difluoromethyl)-N-methylindolizine-2-carboxamide;
(S)-di-tert-butyl ((2-((8,9-difluoro-6-oxo-1,4,5,6-tetrahydro-2H-pyrano[3,4-c]isoquinolin-1-yl)(methyl)carbamoyl)-5,6-difluoro-1H-indol-1-yl)methyl)phosphate;
(R)-di-tert-butyl ((2-((8,9-difluoro-6-oxo-1,4,5,6-tetrahydro-2H-pyrano[3,4-c]isoquinolin-1-yl)(methyl)carbamoyl)-5,6-difluoro-1H-indol-1-yl)methyl)phosphate;
(S)-5-chloro-N-(8,9-difluoro-6-oxo-1,4,5,6-tetrahydro-2H-pyrano[3,4-c]isoquinolin-1-yl)-N-methyl-6-(trifluoromethyl)nicotinamide;
(R)-5-chloro-N-(8,9-difluoro-6-oxo-1,4,5,6-tetrahydro-2H-pyrano[3,4-c]isoquinolin-1-yl)-N-methyl-6-(trifluoromethyl)nicotinamide;
(S)-5-chloro-N-(8-fluoro-6-oxo-1,4,5,6-tetrahydro-2H-pyrano[3,4-c]isoquinolin-1-yl)-N-methyl-6-(trifluoromethyl)nicotinamide;
(R)-5-chloro-N-(8-fluoro-6-oxo-1,4,5,6-tetrahydro-2H-pyrano[3,4-c]isoquinolin-1-yl)-N-methyl-6-(trifluoromethyl)nicotinamide;
(S)-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-4-cyano-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-cyano-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-4-bromo-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-4-bromo-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-N-(8,9-difluoro-6-oxo-1,2,3,4,5,6-hexahydrobenzo[c][1,7]naphthyridin-1-yl)-7-(difluoromethyl)-N-methylindolizine-2-carboxamide;
(R)-N-(8,9-difluoro-6-oxo-1,2,3,4,5,6-hexahydrobenzo[c][1,7]naphthyridin-1-yl)-7-(difluoromethyl)-N-methylindolizine-2-carboxamide;
(S)-N-(8,9-difluoro-6-oxo-1,2,3,4,5,6-hexahydrobenzo[c][1,7]naphthyridin-1-yl)-6-(difluoromethyl)-N-methylindolizine-2-carboxamide;
(R)-N-(8,9-difluoro-6-oxo-1,2,3,4,5,6-hexahydrobenzo[c][1,7]naphthyridin-1-yl)-6-(difluoromethyl)-N-methylindolizine-2-carboxamide;
(S)-N-(8-fluoro-6-oxo-1,4,5,6-tetrahydro-2H-pyrano[3,4-c]isoquinolin-1-yl)-N-methylindolizine-6-carboxamide;
(R)-N-(8-fluoro-6-oxo-1,4,5,6-tetrahydro-2H-pyrano[3,4-c]isoquinolin-1-yl)-N-methylindolizine-6-carboxamide;
(S)-N-(8,9-difluoro-6-oxo-1,2,3,4,5,6-hexahydrobenzo[c][1,7]naphthyridin-1-yl)-N-methylindolizine-6-carboxamide;
(R)-N-(8,9-difluoro-6-oxo-1,2,3,4,5,6-hexahydrobenzo[c][1,7]naphthyridin-1-yl)-N-methylindolizine-6-carboxamide;
(S)-4-ethyl-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-ethyl-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-5-cyano-N-(8-fluoro-6-oxo-1,4,5,6-tetrahydro-2H-pyrano[3,4-c]isoquinolin-1-yl)-N-methyl-1H-indole-2-carboxamide;
(R)-5-cyano-N-(8-fluoro-6-oxo-1,4,5,6-tetrahydro-2H-pyrano[3,4-c]isoquinolin-1-yl)-N-methyl-1H-indole-2-carboxamide;
(S)-4-cyano-N-(8,9-difluoro-6-oxo-1,2,3,4,5,6-hexahydrobenzo[c][1,7]naphthyridin-1-yl)-6-fluoro-N-methyl-1H-indole-2-carboxamide;
(R)-4-cyano-N-(8,9-difluoro-6-oxo-1,2,3,4,5,6-hexahydrobenzo[c][1,7]naphthyridin-1-yl)-6-fluoro-N-methyl-1H-indole-2-carboxamide;
(S)-N-(8,9-difluoro-6-oxo-1,2,3,4,5,6-hexahydrobenzo[c][1,7]naphthyridin-1-yl)-4-ethyl-6-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-4-ethyl-6-fluoro-N-methyl-1H-indole-2-carboxamide;
(S)-N-(8,9-difluoro-6-oxo-1,2,3,4,5,6-hexahydrobenzo[c][1,7]naphthyridin-1-yl)-N-methyl-5-(methylsulfonyl)-1H-indole-2-carboxamide;
(R)-N-(8,9-difluoro-6-oxo-1,2,3,4,5,6-hexahydrobenzo[c][1,7]naphthyridin-1-yl)-N-methyl-5-(methylsulfonyl)-1H-indole-2-carboxamide;
(S)-5-cyano-N-(8,9-difluoro-6-oxo-1,2,3,4,5,6-hexahydrobenzo[c][1,7]naphthyridin-1-yl)-N-methyl-1H-indole-2-carboxamide;
(R)-5-cyano-N-(8,9-difluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-8-(difluoromethyl)-N-methylindolizine-2-carboxamide;
(R)-N-(8,9-difluoro-6-oxo-1,4,5,6-tetrahydro-2H-pyrano[3,4-c]isoquinolin-1-yl)-8-(difluoromethyl)-N-methylindolizine-2-carboxamide;
(S)-N-(8,9-difluoro-6-oxo-1,4,5,6-tetrahydro-2H-pyrano[3,4-c]isoquinolin-1-yl)-N-methyl-2,3-dihydro-1H-indene-5-carboxamide;
(R)-N-(8,9-difluoro-6-oxo-1,4,5,6-tetrahydro-2H-pyrano[3,4-c]isoquinolin-1-yl)-N-methyl-2,3-dihydro-1H-indene-5-carboxamide;
(S)-N-(8,9-difluoro-6-oxo-1,4,5,6-tetrahydro-2H-pyrano[3,4-c]isoquinolin-1-yl)-N-methylbenzo[d]oxazole-6-carboxamide;
(R)-N-(8,9-difluoro-6-oxo-1,4,5,6-tetrahydro-2H-pyrano[3,4-c]isoquinolin-1-yl)-N-methylbenzo[d]oxazole-6-carboxamide;
(S)-4,6-dichloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(R)-4,6-dichloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
5,6-difluoro-N-methyl-N-((1S)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1S,4S)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1S,4R)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1R)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1R,4S)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1R,4R)-4,8,9-trifluoro-6-oxo-1,4,5,6-tetrahydro-2H-pyrano[3,4-c]isoquinolin-1-yl)-1H-indole-2-carboxamide;
(S)-N-(8,9-difluoro-6-oxo-1,4,5,6-tetrahydro-2H-pyrano[3,4-c]isoquinolin-1-yl)-5-fluoro-N-methyl-6-(trifluoromethyl)nicotinamide;
(R)-N-(8,9-difluoro-6-oxo-1,4,5,6-tetrahydro-2H-pyrano[3,4-c]isoquinolin-1-yl)-5-fluoro-N-methyl-6-(trifluoromethyl)nicotinamide;
(S)-5-fluoro-N-(8-fluoro-6-oxo-1,4,5,6-tetrahydro-2H-pyrano[3,4-c]isoquinolin-1-yl)-N-methyl-6-(trifluoromethyl)nicotinamide;
(R)-5-fluoro-N-(8-fluoro-6-oxo-1,4,5,6-tetrahydro-2H-pyrano[3,4-c]isoquinolin-1-yl)-N-methyl-6-(trifluoromethyl)nicotinamide;
(S)-3-chloro-N-(8,9-difluoro-6-oxo-1,4,5,6-tetrahydro-2H-pyrano[3,4-c]isoquinolin-1-yl)-N,1-dimethyl-1H-indazole-5-carboxamide;
(R)-3-chloro-N-(8,9-difluoro-6-oxo-1,4,5,6-tetrahydro-2H-pyrano[3,4-c]isoquinolin-1-yl)-N,1-dimethyl-1H-indazole-5-carboxamide;
(S)-3-chloro-N-(8,9-difluoro-6-oxo-1,4,5,6-tetrahydro-2H-pyrano[3,4-c]isoquinolin-1-yl)-1-ethyl-N-methyl-1H-indazole-5-carboxamide;
(R)-3-chloro-N-(8,9-difluoro-6-oxo-1,4,5,6-tetrahydro-2H-pyrano[3,4-c]isoquinolin-1-yl)-1-ethyl-N-methyl-1H-indazole-5-carboxamide;
(S)-N-(8,9-difluoro-6-oxo-1,4,5,6-tetrahydro-2H-pyrano[3,4-c]isoquinolin-1-yl)-1-(difluoromethyl)-N,3-dimethyl-1H-indazole-5-carboxamide;
(R)-N-(8,9-difluoro-6-oxo-1,4,5,6-tetrahydro-2H-pyrano[3,4-c]isoquinolin-1-yl)-1-(difluoromethyl)-N,3-dimethyl-1H-indazole-5-carboxamide;
(S)-5-chloro-N-(8,9-difluoro-6-oxo-1,2,3,4,5,6-hexahydrobenzo[c][1,7]naphthyridin-1-yl)-N-methyl-6-(trifluoromethyl)nicotinamide;
(R)-5-chloro-N-(8,9-difluoro-6-oxo-1,2,3,4,5,6-hexahydrobenzo[c][1,7]naphthyridin-1-yl)-N-methyl-6-(trifluoromethyl)nicotinamide;
(S)-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
(R)-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
(S)-N-(8-fluoro-6-oxo-1,4,5,6-tetrahydro-2H-pyrano[3,4-c]isoquinolin-1-yl)-N,4-dimethyl-1H-indole-2-carboxamide;
(R)-N-(8-fluoro-6-oxo-1,4,5,6-tetrahydro-2H-pyrano[3,4-c]isoquinolin-1-yl)-N,4-dimethyl-1H-indole-2-carboxamide;
(S)-4-chloro-N-(8-fluoro-6-oxo-1,4,5,6-tetrahydro-2H-pyrano[3,4-c]isoquinolin-1-yl)-N-methyl-1H-indole-2-carboxamide;
(R)-4-chloro-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methylbenzo[d]oxazole-5-carboxamide;
(R)-N-(8,9-difluoro-6-oxo-1,4,5,6-tetrahydro-2H-pyrano[3,4-c]isoquinolin-1-yl)-N-methylbenzo[d]oxazole-5-carboxamide;
(S)-N-(8,9-difluoro-6-oxo-1,4,5,6-tetrahydro-2H-pyrano[3,4-c]isoquinolin-1-yl)-N-methylbenzo[d]thiazole-5-carboxamide;
(R)-N-(8,9-difluoro-6-oxo-1,4,5,6-tetrahydro-2H-pyrano[3,4-c]isoquinolin-1-yl)-N-methylbenzo[d]thiazole-5-carboxamide;
(S)-N-(8,9-difluoro-6-oxo-1,4,5,6-tetrahydro-2H-pyrano[3,4-c]isoquinolin-1-yl)-N-methylbenzo[d]thiazole-6-carboxamide;
(R)-N-(8,9-difluoro-6-oxo-1,4,5,6-tetrahydro-2H-pyrano[3,4-c]isoquinolin-1-yl)-N-methylbenzo[d]thiazole-6-carboxamide;
(S)-N-(8,9-difluoro-6-oxo-1,4,5,6-tetrahydro-2H-pyrano[3,4-c]isoquinolin-1-yl)-N-methyl-1H-indazole-5-carboxamide;
(R)-N-(8,9-difluoro-6-oxo-1,4,5,6-tetrahydro-2H-pyrano[3,4-c]isoquinolin-1-yl)-N-methyl-1H-indazole-5-carboxamide;
(S)-N-(8,9-difluoro-6-oxo-1,2,3,4,5,6-hexahydrobenzo[c][1,7]naphthyridin-1-yl)-8-(difluoromethyl)-N-methylindolizine-2-carboxamide;
(R)-N-(8,9-difluoro-6-oxo-1,2,3,4,5,6-hexahydrobenzo[c][1,7]naphthyridin-1-yl)-8-(difluoromethyl)-N-methylindolizine-2-carboxamide;
(S)-N-(8,9-difluoro-6-oxo-1,2,3,4,5,6-hexahydrobenzo[c][1,7]naphthyridin-1-yl)-5-(difluoromethyl)-N-methylindolizine-2-carboxamide;
(R)-N-(8,9-difluoro-6-oxo-1,2,3,4,5,6-hexahydrobenzo[c][1,7]naphthyridin-1-yl)-5-(difluoromethyl)-N-methylindolizine-2-carboxamide;
(S)-N-(8,9-difluoro-6-oxo-1,2,3,4,5,6-hexahydrobenzo[c][1,7]naphthyridin-1-yl)-5-fluoro-N-methyl-6-(trifluoromethyl)nicotinamide;
(R)-N-(8,9-difluoro-6-oxo-1,2,3,4,5,6-hexahydrobenzo[c][1,7]naphthyridin-1-yl)-5-fluoro-N-methyl-6-(trifluoromethyl)nicotinamide;
(S)-N-(8,9-difluoro-6-oxo-1,4,5,6-tetrahydro-2H-pyrano[3,4-c]isoquinolin-1-yl)-3-(difluoromethyl)-N,1-dimethyl-1H-indazole-5-carboxamide;
(R)-N-(8,9-difluoro-6-oxo-1,4,5,6-tetrahydro-2H-pyrano[3,4-c]isoquinolin-1-yl)-3-(difluoromethyl)-N,1-dimethyl-1H-indazole-5-carboxamide;
(S)-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
(R)-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
(S)-4-chloro-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-chloro-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-4-chloro-N-(8,9-difluoro-6-oxo-1,2,3,4,5,6-hexahydrobenzo[c][1,7]naphthyridin-1-yl)-N-methyl-1H-indole-2-carboxamide;
(R)-4-chloro-N-(8,9-difluoro-6-oxo-1,2,3,4,5,6-hexahydrobenzo[c][1,7]naphthyridin-1-yl)-N-methyl-1H-indole-2-carboxamide;
(S)-N-(8-fluoro-6-oxo-1,4,5,6-tetrahydro-2H-pyrano[3,4-c]isoquinolin-1-yl)-N-methyl-2,3-dihydro-1H-indene-5-carboxamide;
(R)-N-(8-fluoro-6-oxo-1,4,5,6-tetrahydro-2H-pyrano[3,4-c]isoquinolin-1-yl)-N-methyl-2,3-dihydro-1H-indene-5-carboxamide;
(S)-N-(8-fluoro-6-oxo-1,4,5,6-tetrahydro-2H-pyrano[3,4-c]isoquinolin-1-yl)-N-methylbenzo[d]thiazole-5-carboxamide;
(R)-N-(8-fluoro-6-oxo-1,4,5,6-tetrahydro-2H-pyrano[3,4-c]isoquinolin-1-yl)-N-methylbenzo[d]thiazole-5-carboxamide;
(S)-N-(8-fluoro-6-oxo-1,4,5,6-tetrahydro-2H-pyrano[3,4-c]isoquinolin-1-yl)-N-methylbenzo[d]thiazole-6-carboxamide;
(R)-N-(8-fluoro-6-oxo-1,4,5,6-tetrahydro-2H-pyrano[3,4-c]isoquinolin-1-yl)-N-methylbenzo[d]thiazole-6-carboxamide;
(S)-N-(8-fluoro-6-oxo-1,4,5,6-tetrahydro-2H-pyrano[3,4-c]isoquinolin-1-yl)-N-methyl-1H-indazole-5-carboxamide;
(R)-N-(8-fluoro-6-oxo-1,4,5,6-tetrahydro-2H-pyrano[3,4-c]isoquinolin-1-yl)-N-methyl-1H-indazole-5-carboxamide;
(S)-N-(8,9-difluoro-6-oxo-1,4,5,6-tetrahydro-2H-pyrano[3,4-c]isoquinolin-1-yl)-N-methyl-1H-indazole-6-carboxamide;
(R)-N-(8,9-difluoro-6-oxo-1,4,5,6-tetrahydro-2H-pyrano[3,4-c]isoquinolin-1-yl)-N-methyl-1H-indazole-6-carboxamide;
(S)-N-(8,9-difluoro-6-oxo-1,4,5,6-tetrahydro-2H-pyrano[3,4-c]isoquinolin-1-yl)-N-methyl-1H-benzo[d]imidazole-6-carboxamide;
(R)-N-(8,9-difluoro-6-oxo-1,4,5,6-tetrahydro-2H-pyrano[3,4-c]isoquinolin-1-yl)-N-methyl-1H-benzo[d]imidazole-6-carboxamide;
(R)-2-chloro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-2-chloro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-2-chloro-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
(S)-2-chloro-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
(R)-6-fluoro-N-(8-fluoro-6-oxo-1,4,5,6-tetrahydro-2H-pyrano[3,4-c]isoquinolin-1-yl)-N,4-dimethyl-1H-indole-2-carboxamide;
(S)-6-fluoro-N-(8-fluoro-6-oxo-1,4,5,6-tetrahydro-2H-pyrano[3,4-c]isoquinolin-1-yl)-N,4-dimethyl-1H-indole-2-carboxamide;
(R)-N-(8,9-difluoro-6-oxo-1,2,3,4,5,6-hexahydrobenzo[c][1,7]naphthyridin-1-yl)-N,4-dimethyl-1H-indole-2-carboxamide;
(S)-N-(8,9-difluoro-6-oxo-1,2,3,4,5,6-hexahydrobenzo[c][1,7]naphthyridin-1-yl)-N,4-dimethyl-1H-indole-2-carboxamide;
(R)-N-(8,9-difluoro-6-oxo-1,2,3,4,5,6-hexahydrobenzo[c][1,7]naphthyridin-1-yl)-6-fluoro-N,4-dimethyl-1H-indole-2-carboxamide;
(S)-N-(8,9-difluoro-6-oxo-1,2,3,4,5,6-hexahydrobenzo[c][1,7]naphthyridin-1-yl)-6-fluoro-N,4-dimethyl-1H-indole-2-carboxamide;
(R)-4-chloro-N-(8,9-difluoro-6-oxo-1,2,3,4,5,6-hexahydrobenzo[c][1,7]naphthyridin-1-yl)-6-fluoro-N-methyl-1H-indole-2-carboxamide;
(S)-4-chloro-N-(8,9-difluoro-6-oxo-1,2,3,4,5,6-hexahydrobenzo[c][1,7]naphthyridin-1-yl)-6-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-N-methylbenzo[d]thiazole-5-carboxamide;
(S)-N-(8,9-difluoro-6-oxo-1,2,3,4,5,6-hexahydrobenzo[c][1,7]naphthyridin-1-yl)-N-methylbenzo[d]thiazole-5-carboxamide;
(R)-N-(8,9-difluoro-6-oxo-1,2,3,4,5,6-hexahydrobenzo[c][1,7]naphthyridin-1-yl)-N-methylbenzo[d]thiazole-6-carboxamide;
(S)-N-(8,9-difluoro-6-oxo-1,2,3,4,5,6-hexahydrobenzo[c][1,7]naphthyridin-1-yl)-N-methylbenzo[d]thiazole-6-carboxamide;
(R)-N-(8,9-difluoro-6-oxo-1,2,3,4,5,6-hexahydrophenanthridin-1-yl)-N-methyl-1H-indole-2-carboxamide;
(S)-N-(8,9-difluoro-6-oxo-1,2,3,4,5,6-hexahydrophenanthridin-1-yl)-N-methyl-1H-indole-2-carboxamide;
(S)-2-cyano-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-2-cyano-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-N-(8-fluoro-6-oxo-1,4,5,6-tetrahydro-2H-pyrano[3,4-c]isoquinolin-1-yl)-N-methyl-1H-indazole-6-carboxamide;
(S)-N-(8-fluoro-6-oxo-1,4,5,6-tetrahydro-2H-pyrano[3,4-c]isoquinolin-1-yl)-N-methyl-1H-indazole-6-carboxamide;
(R)-N-(8,9-difluoro-6-oxo-1,2,3,4,5,6-hexahydrobenzo[c][1,7]naphthyridin-1-yl)-N-methyl-2,3-dihydro-1H-indene-5-carboxamide;
(S)-N-(8,9-difluoro-6-oxo-1,2,3,4,5,6-hexahydrobenzo[c][1,7]naphthyridin-1-yl)-N-methyl-2,3-dihydro-1H-indene-5-carboxamide;
(R)-N-(8,9-difluoro-6-oxo-1,2,3,4,5,6-hexahydrobenzo[c][1,7]naphthyridin-1-yl)-N-methyl-1H-indazole-5-carboxamide;
(S)-N-(8,9-difluoro-6-oxo-1,2,3,4,5,6-hexahydrobenzo[c][1,7]naphthyridin-1-yl)-N-methyl-1H-indazole-5-carboxamide;
(R)-N-(8-fluoro-6-oxo-1,2,3,4,5,6-hexahydrophenanthridin-1-yl)-N-methyl-1H-indole-2-carboxamide;
(S)-N-(8-fluoro-6-oxo-1,2,3,4,5,6-hexahydrophenanthridin-1-yl)-N-methyl-1H-indole-2-carboxamide;
(S)-2-chloro-N-(8,9-difluoro-4,6-dioxo-1,4,5,6-tetrahydro-2H-pyrano[3,4-c]isoquinolin-1-yl)-N-methyl-4H-thieno[3,2-b]pyrrole-5-carboxamide;
(R)-2-chloro-N-(8,9-difluoro-4,6-dioxo-1,4,5,6-tetrahydro-2H-pyrano[3,4-c]isoquinolin-1-yl)-N-methyl-4H-thieno[3,2-b]pyrrole-5-carboxamide;
(S)-N-(8,9-difluoro-4,6-dioxo-1,4,5,6-tetrahydro-2H-pyrano[3,4-c]isoquinolin-1-yl)-6-(difluoromethyl)-5-fluoro-N-methyl-1H-indole-2-carboxamide;
(R)-N-(8,9-difluoro-4,6-dioxo-1,4,5,6-tetrahydro-2H-pyrano[3,4-c]isoquinolin-1-yl)-6-(difluoromethyl)-5-fluoro-N-methyl-1H-indole-2-carboxamide;
(R)-6-(difluoromethyl)-5-fluoro-N-(8-fluoro-6-oxo-1,2,3,4,5,6-hexahydrophenanthridin-1-yl)-N-methyl-1H-indole-2-carboxamide;
(S)-6-(difluoromethyl)-5-fluoro-N-(8-fluoro-6-oxo-1,2,3,4,5,6-hexahydrophenanthridin-1-yl)-N-methyl-1H-indole-2-carboxamide;
(R)-N-(8,9-difluoro-6-oxo-1,2,3,4,5,6-hexahydrophenanthridin-1-yl)-5-fluoro-N-methyl-1H-indole-2-carboxamide;
(S)-N-(8,9-difluoro-6-oxo-1,2,3,4,5,6-hexahydrophenanthridin-1-yl)-5-fluoro-N-methyl-1H-indole-2-carboxamide;
(R)-N-(8,9-difluoro-6-oxo-1,2,3,4,5,6-hexahydrophenanthridin-1-yl)-5,6-difluoro-N-methyl-1H-indole-2-carboxamide;
(S)-N-(8,9-difluoro-6-oxo-1,2,3,4,5,6-hexahydrophenanthridin-1-yl)-5,6-difluoro-N-methyl-1H-indole-2-carboxamide;
(R)-N-(8,9-difluoro-6-oxo-1,2,3,4,5,6-hexahydrophenanthridin-1-yl)-6-(difluoromethyl)-5-fluoro-N-methyl-1H-indole-2-carboxamide;
(S)-N-(8,9-difluoro-6-oxo-1,2,3,4,5,6-hexahydrophenanthridin-1-yl)-6-(difluoromethyl)-5-fluoro-N-methyl-1H-indole-2-carboxamide;
(R)-N-(8,9-difluoro-6-oxo-1,2,3,4,5,6-hexahydrophenanthridin-1-yl)-4-(difluoromethyl)-6-fluoro-N-methyl-1H-indole-2-carboxamide;
(S)-N-(8,9-difluoro-6-oxo-1,2,3,4,5,6-hexahydrophenanthridin-1-yl)-4-(difluoromethyl)-6-fluoro-N-methyl-1H-indole-2-carboxamide;
(R)-N-(8,9-difluoro-6-oxo-1,2,3,4,5,6-hexahydrophenanthridin-1-yl)-N,4-dimethyl-1H-indole-2-carboxamide;
(S)-N-(8,9-difluoro-6-oxo-1,2,3,4,5,6-hexahydrophenanthridin-1-yl)-N,4-dimethyl-1H-indole-2-carboxamide;
(R)-N-(8,9-difluoro-6-oxo-1,2,3,4,5,6-hexahydrophenanthridin-1-yl)-6-fluoro-N,4-dimethyl-1H-indole-2-carboxamide;
(S)-N-(8,9-difluoro-6-oxo-1,2,3,4,5,6-hexahydrophenanthridin-1-yl)-6-fluoro-N,4-dimethyl-1H-indole-2-carboxamide;
(S)-1-((4-bromo-3-fluorobenzyl)(methyl)amino)-8,9-difluoro-1,5-dihydro-2H-pyrano[3,4-c]isoquinolin-6(4H)-one;
(R)-1-((4-bromo-3-fluorobenzyl)(methyl)amino)-8,9-difluoro-1,5-dihydro-2H-pyrano[3,4-c]isoquinolin-6(4H)-one;
2-chloro-N-((1S)-8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
(S,R)-2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
(S,S)-2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
2-chloro-N-((1R)-8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
(R,R)-2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
(R,S)-2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
N-((1R)-8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(R,R)-N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(R,S)-N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
N-((1S)-8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(S,S)-N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(S,R)-N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(R)-5-fluoro-N-(8-fluoro-6-oxo-1,2,3,4,5,6-hexahydrophenanthridin-1-yl)-N-methyl-1H-indole-2-carboxamide;
(S)-5-fluoro-N-(8-fluoro-6-oxo-1,2,3,4,5,6-hexahydrophenanthridin-1-yl)-N-methyl-1H-indole-2-carboxamide;
(R)-5,6-difluoro-N-(8-fluoro-6-oxo-1,2,3,4,5,6-hexahydrophenanthridin-1-yl)-N-methyl-1H-indole-2-carboxamide;
(S)-5,6-difluoro-N-(8-fluoro-6-oxo-1,2,3,4,5,6-hexahydrophenanthridin-1-yl)-N-methyl-1H-indole-2-carboxamide;
(R)-N-(8-fluoro-6-oxo-1,2,3,4,5,6-hexahydrophenanthridin-1-yl)-N,4-dimethyl-1H-indole-2-carboxamide;
(S)-N-(8-fluoro-6-oxo-1,2,3,4,5,6-hexahydrophenanthridin-1-yl)-N,4-dimethyl-1H-indole-2-carboxamide;
(R)-6-fluoro-N-(8-fluoro-6-oxo-1,2,3,4,5,6-hexahydrophenanthridin-1-yl)-N,4-dimethyl-1H-indole-2-carboxamide;
(S)-6-fluoro-N-(8-fluoro-6-oxo-1,2,3,4,5,6-hexahydrophenanthridin-1-yl)-N,4-dimethyl-1H-indole-2-carboxamide;
(R)-4-(difluoromethyl)-6-fluoro-N-(8-fluoro-6-oxo-1,2,3,4,5,6-hexahydrophenanthridin-1-yl)-N-methyl-1H-indole-2-carboxamide;
(S)-4-(difluoromethyl)-6-fluoro-N-(8-fluoro-6-oxo-1,2,3,4,5,6-hexahydrophenanthridin-1-yl)-N-methyl-1H-indole-2-carboxamide;
(S)-1-(((5,6-difluoro-1H-indol-2-yl)methyl)(methyl)amino)-8,9-difluoro-1,5-dihydro-2H-pyrano[3,4-c]isoquinolin-6(4H)-one;
(R)-1-(((5,6-difluoro-1H-indol-2-yl)methyl)(methyl)amino)-8,9-difluoro-1,5-dihydro-2H-pyrano[3,4-c]isoquinolin-6(4H)-one;
(S)-N-(8,9-difluoro-6-oxo-1,2,3,4,5,6-hexahydrobenzo[c][1,7]naphthyridin-1-yl)-N-methyl-1H-indazole-6-carboxamide;
(R)-N-(8,9-difluoro-6-oxo-1,2,3,4,5,6-hexahydrobenzo[c][1,7]naphthyridin-1-yl)-N-methyl-1H-indazole-6-carboxamide;
(S)-2-fluoro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-2-fluoro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-N-(8,9-difluoro-6-oxo-1,2,3,4,5,6-hexahydrobenzo[c][1,7]naphthyridin-1-yl)-2-fluoro-N-methyl-4H-thieno[3,2-b]pyrrole-5-carboxamide;
(R)-N-(8,9-difluoro-6-oxo-1,2,3,4,5,6-hexahydrobenzo[c][1,7]naphthyridin-1-yl)-2-fluoro-N-methyl-4H-thieno[3,2-b]pyrrole-5-carboxamide;
N-((1R)-8,9-difluoro-4-hydroxy-6-oxo-1,2,3,4,5,6-hexahydrophenanthridin-1-yl)-5-fluoro-N-methyl-1H-indole-2-carboxamide;
N-((1S)-8,9-difluoro-4-hydroxy-6-oxo-1,2,3,4,5,6-hexahydrophenanthridin-1-yl)-5-fluoro-N-methyl-1H-indole-2-carboxamide;
(R,R)-N-(8,9-difluoro-4-hydroxy-6-oxo-1,2,3,4,5,6-hexahydrophenanthridin-1-yl)-5-fluoro-N-methyl-1H-indole-2-carboxamide;
(S,R)-N-(8,9-difluoro-4-hydroxy-6-oxo-1,2,3,4,5,6-hexahydrophenanthridin-1-yl)-5-fluoro-N-methyl-1H-indole-2-carboxamide;
(S,S)-N-(8,9-difluoro-4-hydroxy-6-oxo-1,2,3,4,5,6-hexahydrophenanthridin-1-yl)-5-fluoro-N-methyl-1H-indole-2-carboxamide;
(R,S)-N-(8,9-difluoro-4-hydroxy-6-oxo-1,2,3,4,5,6-hexahydrophenanthridin-1-yl)-5-fluoro-N-methyl-1H-indole-2-carboxamide;
N-((1R)-8,9-difluoro-4-hydroxy-6-oxo-1,2,3,4,5,6-hexahydrophenanthridin-1-yl)-5,6-difluoro-N-methyl-1H-indole-2-carboxamide;
N-((1S)-8,9-difluoro-4-hydroxy-6-oxo-1,2,3,4,5,6-hexahydrophenanthridin-1-yl)-5,6-difluoro-N-methyl-1H-indole-2-carboxamide;
(S,R)-N-(8,9-difluoro-4-hydroxy-6-oxo-1,2,3,4,5,6-hexahydrophenanthridin-1-yl)-5,6-difluoro-N-methyl-1H-indole-2-carboxamide;
(R,R)-N-(8,9-difluoro-4-hydroxy-6-oxo-1,2,3,4,5,6-hexahydrophenanthridin-1-yl)-5,6-difluoro-N-methyl-1H-indole-2-carboxamide;
(R,S)-N-(8,9-difluoro-4-hydroxy-6-oxo-1,2,3,4,5,6-hexahydrophenanthridin-1-yl)-5,6-difluoro-N-methyl-1H-indole-2-carboxamide;
(S,S)-N-(8,9-difluoro-4-hydroxy-6-oxo-1,2,3,4,5,6-hexahydrophenanthridin-1-yl)-5,6-difluoro-N-methyl-1H-indole-2-carboxamide;
N-((1R)-8,9-difluoro-4-hydroxy-6-oxo-1,2,3,4,5,6-hexahydrophenanthridin-1-yl)-N-methyl-1H-indole-2-carboxamide;
N-((1S)-8,9-difluoro-4-hydroxy-6-oxo-1,2,3,4,5,6-hexahydrophenanthridin-1-yl)-N-methyl-1H-indole-2-carboxamide;
(S,R)-N-(8,9-difluoro-4-hydroxy-6-oxo-1,2,3,4,5,6-hexahydrophenanthridin-1-yl)-N-methyl-1H-indole-2-carboxamide;
(R,R)-N-(8,9-difluoro-4-hydroxy-6-oxo-1,2,3,4,5,6-hexahydrophenanthridin-1-yl)-N-methyl-1H-indole-2-carboxamide;
(R,S)-N-(8,9-difluoro-4-hydroxy-6-oxo-1,2,3,4,5,6-hexahydrophenanthridin-1-yl)-N-methyl-1H-indole-2-carboxamide;
(S,S)-N-(8,9-difluoro-4-hydroxy-6-oxo-1,2,3,4,5,6-hexahydrophenanthridin-1-yl)-N-methyl-1H-indole-2-carboxamide;
(R)-N-(8,9-difluoro-6-oxo-1,2,3,4,5,6-hexahydrobenzo[c][1,7]naphthyridin-1-yl)-N-methylindolizine-2-carboxamide;
(S)-N-(8,9-difluoro-6-oxo-1,2,3,4,5,6-hexahydrobenzo[c][1,7]naphthyridin-1-yl)-N-methylindolizine-2-carboxamide;
(R)-8-chloro-N-(8,9-difluoro-6-oxo-1,2,3,4,5,6-hexahydrobenzo[c][1,7]naphthyridin-1-yl)-N-methylindolizine-2-carboxamide;
(S)-8-chloro-N-(8,9-difluoro-6-oxo-1,2,3,4,5,6-hexahydrobenzo[c][1,7]naphthyridin-1-yl)-N-methylindolizine-2-carboxamide;
(S)-N-(8,9-Difluoro-6-oxo-1,4,5,6-tetrahydro-2H-pyrano[3,4-c]isoquinolin-1-yl)-2,2-difluoro-N-methyl-2-phenylacetamide;
(R)-N-(8,9-Difluoro-6-oxo-1,4,5,6-tetrahydro-2H-pyrano[3,4-c]isoquinolin-1-yl)-2,2-difluoro-N-methyl-2-phenylacetamide;
(S)-N-(8,9-Difluoro-6-oxo-1,4,5,6-tetrahydro-2H-pyrano[3,4-c]isoquinolin-1-yl-1-d)-5,6-difluoro-N-(methyl-13C-d3)-1H-indole-2-carboxamide;
(R)-N-(8,9-Difluoro-6-oxo-1,4,5,6-tetrahydro-2H-pyrano[3,4-c]isoquinolin-1-yl-1-d)-5,6-difluoro-N-(methyl-13C-d3)-1H-indole-2-carboxamide;
(S)-N-(8,9-Difluoro-6-oxo-1,4,5,6-tetrahydro-2H-pyrano[3,4-c]isoquinolin-1-yl-1-d)-6-(difluoromethyl)-5-fluoro-N-(methyl-13C-d3)-1H-indole-2-carboxamide;
(R)-N-(8,9-Difluoro-6-oxo-1,4,5,6-tetrahydro-2H-pyrano[3,4-c]isoquinolin-1-yl-1-d)-6-(difluoromethyl)-5-fluoro-N-(methyl-13C-d3)-1H-indole-2-carboxamide;
(S)-2-Chloro-N-(8,9-difluoro-6-oxo-1,4,5,6-tetrahydro-2H-pyrano[3,4-c]isoquinolin-1-yl-1-d)-N-(methyl-13C-d3)-4H-thieno[3,2-b]pyrrole-5-carboxamide; and
(R)-2-Chloro-N-(8,9-difluoro-6-oxo-1,4,5,6-tetrahydro-2H-pyrano[3,4-c]isoquinolin-1-yl-1-d)-N-(methyl-13C-d3)-4H-thieno[3,2-b]pyrrole-5-carboxamide;
   or a salt, solvate, prodrug, isotopically labelled, stereoisomer, any mixture of stereoisomers, tautomer, and/or any mixture of tautomers thereof.

The compounds of the disclosure may possess one or more stereocenters, and each stereocenter may exist independently in either the (R)- or (S)-configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds of the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

### Salts

The compounds described herein may form salts with acids or bases, and such salts are included in the present disclosure. The term “salts” embraces addition salts of free acids or bases that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the disclosure.

Suitable pharmaceutically acceptable base addition salts of compounds of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Combination Therapies

In one aspect, the compounds of the disclosure are useful within the methods of the disclosure in combination with one or more additional agents useful for treating, ameliorating, and/or preventing HBV and/or HDV infections. These additional agents may comprise compounds or compositions identified herein, or compounds (e.g., commercially available compounds) known to treat, prevent, or reduce the symptoms of HBV and/or HDV infections.

Non-limiting examples of one or more additional agents useful for treating, ameliorating, and/or preventing HBV and/or HDV infections include: (a) reverse transcriptase inhibitors; (b) capsid inhibitors; (c) cccDNA formation inhibitors; (d) RNA destabilizers; (e) oligomeric nucleotides targeted against the HBV genome; (f) immunostimulators, such as checkpoint inhibitors (e.g., PD-L1 inhibitors); (g) GalNAc-siRNA conjugates targeted against an HBV gene transcript; and (h) therapeutic vaccine.

### (a) Reverse Transcriptase Inhibitors

In certain embodiments, the reverse transcriptase inhibitor is a reverse-transcriptase inhibitor (NARTI or NRTI). In other embodiments, the reverse transcriptase inhibitor is a nucleotide analog reverse-transcriptase inhibitor (NtARTI or NtRTI).

Reported reverse transcriptase inhibitors include, but are not limited to, entecavir, clevudine, telbivudine, lamivudine, adefovir, and tenofovir, tenofovir disoproxil, tenofovir alafenamide, adefovir dipovoxil, (1R,2R,3R,5R)-3-(6-amino-9H-9-purinyl)-2-fluoro-5-(hydroxymethyl)-4-methylenecyclopentan-1-ol (described in U.S. Pat. No. 8,816,074, incorporated herein in its entirety by reference), emtricitabine, abacavir, elvucitabine, ganciclovir, lobucavir, famciclovir, penciclovir, and amdoxovir.

Reported reverse transcriptase inhibitors further include, but are not limited to, entecavir, lamivudine, and (1R,2R,3R,5R)-3-(6-amino-9H-9-purinyl)-2-fluoro-5-(hydroxymethyl)-4-methylenecyclopentan-1-ol.

Reported reverse transcriptase inhibitors further include, but are not limited to, a covalently bound phosphoramidate or phosphonamidate moiety of the above-mentioned reverse transcriptase inhibitors, or as described in for example U.S. Pat. No. 8,816,074, US Patent Application Publications No. US 2011/0245484 A1, and US 2008/0286230A1, all of which incorporated herein in their entireties by reference.

Reported reverse transcriptase inhibitors further include, but are not limited to, nucleotide analogs that comprise a phosphoramidate moiety, such as, for example, methyl ((((1R,3R,4R,5R)-3-(6-amino-9H-purin-9-yl)-4-fluoro-5-hydroxy-2-methylenecyclopentyl) methoxy)(phenoxy)phosphoryl)-(D or L)-alaninate and methyl ((((1R,2R,3R,4R)-3-fluoro-2-hydroxy-5-methylene-4-(6-oxo-1,6-dihydro-9H-purin-9-yl)cyclopentyl)methoxy)(phenoxy)phosphoryl)-(D or L)-alaninate. Also included are the individual diastereomers thereof, which include, for example, methyl ((R)-(((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((S)-(((1R,3R,4R,5R)-3-(6-amino-9H-purin-9-yl)-4-fluoro-5-hydroxy-2-methylenecyclopentyl) methoxy)(phenoxy)phosphoryl)-(D or L)-alaninate.

Reported reverse transcriptase inhibitors further include, but are not limited to, compounds comprising a phosphonamidate moiety, such as, for example, tenofovir alafenamide, as well as those described in U.S. Patent Application Publication No. US 2008/0286230 A1, incorporated herein in its entirety by reference. Methods for preparing stereoselective phosphoramidate or phosphonamidate containing actives are described in, for example, U.S. Pat. No. 8,816,074, as well as U.S. Patent Application Publications No. US 2011/0245484 A1 and US 2008/0286230 A1, all of which incorporated herein in their entireties by reference.

### (b) Capsid Inhibitors

As described herein, the term “capsid inhibitor” includes compounds that are capable of inhibiting the expression and/or function of a capsid protein either directly or indirectly. For example, a capsid inhibitor may include, but is not limited to, any compound that inhibits capsid assembly, induces formation of non-capsid polymers, promotes excess capsid assembly or misdirected capsid assembly, affects capsid stabilization, and/or inhibits encapsidation of RNA (pgRNA). Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In certain embodiments, the inhibitor inhibits the level of rcDNA and downstream products of viral life cycle by at least 5%, at least 10%, at least 20%, at least 50%, at least 75%, or at least 90%.

Reported capsid inhibitors include, but are not limited to, compounds described in International Patent Applications Publication Nos WO 2013006394, WO 2014106019, and WO2014089296, all of which incorporated herein in their entireties by reference.

Reported capsid inhibitors also include, but are not limited to, the following compounds and pharmaceutically acceptable salts and/or solvates thereof: Bay-41-4109 (see Int'l Patent Application Publication No. WO 2013144129), AT-61 (see Int'l Patent Application Publication No. WO 1998033501; and King, et al., 1998, Antimicrob. Agents Chemother. 42(12):3179-3186), DVR-01 and DVR-23 (see Int'l Patent Application Publication No. WO 2013006394; and Campagna, et al., 2013, J. Virol. 87(12):6931, all of which incorporated herein in their entireties by reference.

In addition, reported capsid inhibitors include, but are not limited to, those generally and specifically described in U.S. Patent Application Publication Nos. US 2015/0225355, US 2015/0132258, US 2016/0083383, US 2016/0052921, US 2019/0225593, and Int'l Patent Application Publication Nos. WO 2013096744, WO 2014165128, WO 2014033170, WO 2014033167, WO 2014033176, WO 2014131847, WO 2014161888, WO 2014184350, WO 2014184365, WO 2015059212, WO 2015011281, WO 2015118057, WO 2015109130, WO 2015073774, WO 2015180631, WO 2015138895, WO 2016089990, WO 2017015451, WO 2016183266, WO 2017011552, WO 2017048950, WO2017048954, WO 2017048962, WO 2017064156, WO 2018052967, WO 2018172852, WO 2020023710 and are incorporated herein in their entirety by reference.

(c) cccDNA Formation Inhibitors

Covalently closed circular DNA (cccDNA) is generated in the cell nucleus from viral rcDNA and serves as the transcription template for viral mRNAs. As described herein, the term “cccDNA formation inhibitor” includes compounds that are capable of inhibiting the formation and/or stability of cccDNA either directly or indirectly. For example, a cccDNA formation inhibitor may include, but is not limited to, any compound that inhibits capsid disassembly, rcDNA entry into the nucleus, and/or the conversion of rcDNA into cccDNA. For example, in certain embodiments, the inhibitor detectably inhibits the formation and/or stability of the cccDNA as measured, e.g., using an assay described herein. In certain embodiments, the inhibitor inhibits the formation and/or stability of cccDNA by at least 5%, at least 10%, at least 20%, at least 50%, at least 75%, or at least 90%.

Reported cccDNA formation inhibitors include, but are not limited to, compounds described in Int'l Patent Application Publication No. WO 2013130703, and are incorporated herein in their entirety by reference.

In addition, reported cccDNA formation inhibitors include, but are not limited to, those generally and specifically described in U.S. Patent Application Publication No. US 2015/0038515 A1, and are incorporated herein in their entirety by reference.

### (d) RNA Destabilizer

As used herein, the term “RNA destabilizer” refers to a molecule, or a salt or solvate thereof, that reduces the total amount of HBV RNA in mammalian cell culture or in a live human subject. In a non-limiting example, an RNA destabilizer reduces the amount of the RNA transcript(s) encoding one or more of the following HBV proteins: surface antigen, core protein, RNA polymerase, and e antigen. In certain embodiments, the RNA destabilizer reduces the total amount of HBV RNA in mammalian cell culture or in a live human subject by at least 5%, at least 10%, at least 20%, at least 50%, at least 75%, or at least 90%.

Reported RNA destabilizers include compounds described in U.S. Pat. No. 8,921,381, as well as compounds described in U.S. Patent Application Publication Nos. US 2015/0087659 and US 2013/0303552, all of which are incorporated herein in their entireties by reference.

In addition, reported RNA destabilizers include, but are not limited to, those generally and specifically described in Int'l Patent Application Publication Nos. WO 2015113990, WO 2015173164, US 2016/0122344, WO 2016107832, WO 2016023877, WO 2016128335, WO 2016177655, WO 2016071215, WO 2017013046, WO 2017016921, WO 2017016960, WO 2017017042, WO 2017017043, WO 2017102648, WO 2017108630, WO 2017114812, WO 2017140821, WO 2018085619, and are incorporated herein in their entirety by reference.

### (e) Oligomeric Nucleotides Targeted Against the HBV Genome

Reported oligomeric nucleotides targeted against the HBV genome include, but are not limited to, Arrowhead-ARC-520 (see U.S. Pat. No. 8,809,293; and Wooddell et al., 2013, Molecular Therapy 21(5):973-985, all of which incorporated herein in their entireties by reference).

In certain embodiments, the oligomeric nucleotides can be designed to target one or more genes and/or transcripts of the HBV genome. Oligomeric nucleotide targeted to the HBV genome also include, but are not limited to, isolated, double stranded, siRNA molecules, that each include a sense strand and an antisense strand that is hybridized to the sense strand. In certain embodiments, the siRNA target one or more genes and/or transcripts of the HBV genome.

### (f) Immunostimulators

### Checkpoint Inhibitors

As described herein, the term “checkpoint inhibitor” includes any compound that is capable of inhibiting immune checkpoint molecules that are regulators of the immune system (e.g., stimulate or inhibit immune system activity). For example, some checkpoint inhibitors block inhibitory checkpoint molecules, thereby stimulating immune system function, such as stimulation of T cell activity against cancer cells. A non-limiting example of a checkpoint inhibitor is a PD-L1 inhibitor.

As described herein, the term “PD-L1 inhibitor” includes any compound that is capable of inhibiting the expression and/or function of the protein Programmed Death-Ligand 1 (PD-L1) either directly or indirectly. PD-L1, also known as cluster of differentiation 274 (CD274) or B7 homolog 1 (B7-H1), is a type 1 transmembrane protein that plays a major role in suppressing the adaptive arm of immune system during pregnancy, tissue allograft transplants, autoimmune disease, and hepatitis. PD-L1 binds to its receptor, the inhibitory checkpoint molecule PD-1 (which is found on activated T cells, B cells, and myeloid cells) so as to modulate activation or inhibition of the adaptive arm of immune system. In certain embodiments, the PD-L1 inhibitor inhibits the expression and/or function of PD-L1 by at least 5%, at least 10%, at least 20%, at least 50%, at least 75%, or at least 90%.

Reported PD-L1 inhibitors include, but are not limited to, compounds recited in one of the following patent application publications: US 2018/0057455; US 2018/0057486; WO 2017/106634; WO 2018/026971; WO 2018/045142; WO 2018/118848; WO 2018/119221; WO 2018/119236; WO 2018/119266; WO 2018/119286; WO 2018/121560; WO 2019/076343; WO 2019/087214; and are incorporated herein in their entirety by reference.

### (g) GalNAc-siRNA Conjugates Targeted Against an HBV Gene Transcript

“GalNAc” is the abbreviation for N-acetylgalactosamine, and “siRNA” is the abbreviation for small interfering RNA. An siRNA that targets an HBV gene transcript is covalently bonded to GalNAc in a GalNAc-siRNA conjugate useful in the practice of the present disclosure. While not wishing to be bound by theory, it is believed that GalNAc binds to asialoglycoprotein receptors on hepatocytes thereby facilitating the targeting of the siRNA to the hepatocytes that are infected with HBV. The siRNA enter the infected hepatocytes and stimulate destruction of HBV gene transcripts by the phenomenon of RNA interference.

Examples of GalNAc-siRNA conjugates useful in the practice of this aspect of the present disclosure are set forth in published international application PCT/CA2017/050447 (PCT Application Publication number WO/2017/177326, published on Oct. 19, 2017) which is hereby incorporated by reference in its entirety.

### (h) Therapeutic Vaccines

In certain embodiments, administration of a therapeutic vaccine is useful in the practice of the present disclosure for the treatment of a viral disease in a subject. In certain embodiments, the viral disease is a hepatitis virus. In certain embodiments, the hepatitis virus is at least one selected from the group consisting of hepatitis B virus (HBV) and hepatitis D virus (HDV). In certain embodiments, the subject is a human.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to elsewhere herein may be applied to experimental data to generate a corresponding graph to aid in assessing the effects of the drug combination. The corresponding graphs associated with the equations referred to elsewhere herein are the concentration-effect curve, isobologram curve and combination index curve, respectively.

### Synthesis

The present disclosure further provides methods of preparing compounds of the present disclosure. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatograpy (HPLC), gas chromatography (GC), gel-permeation chromatography (GPC), or thin layer chromatography (TLC).

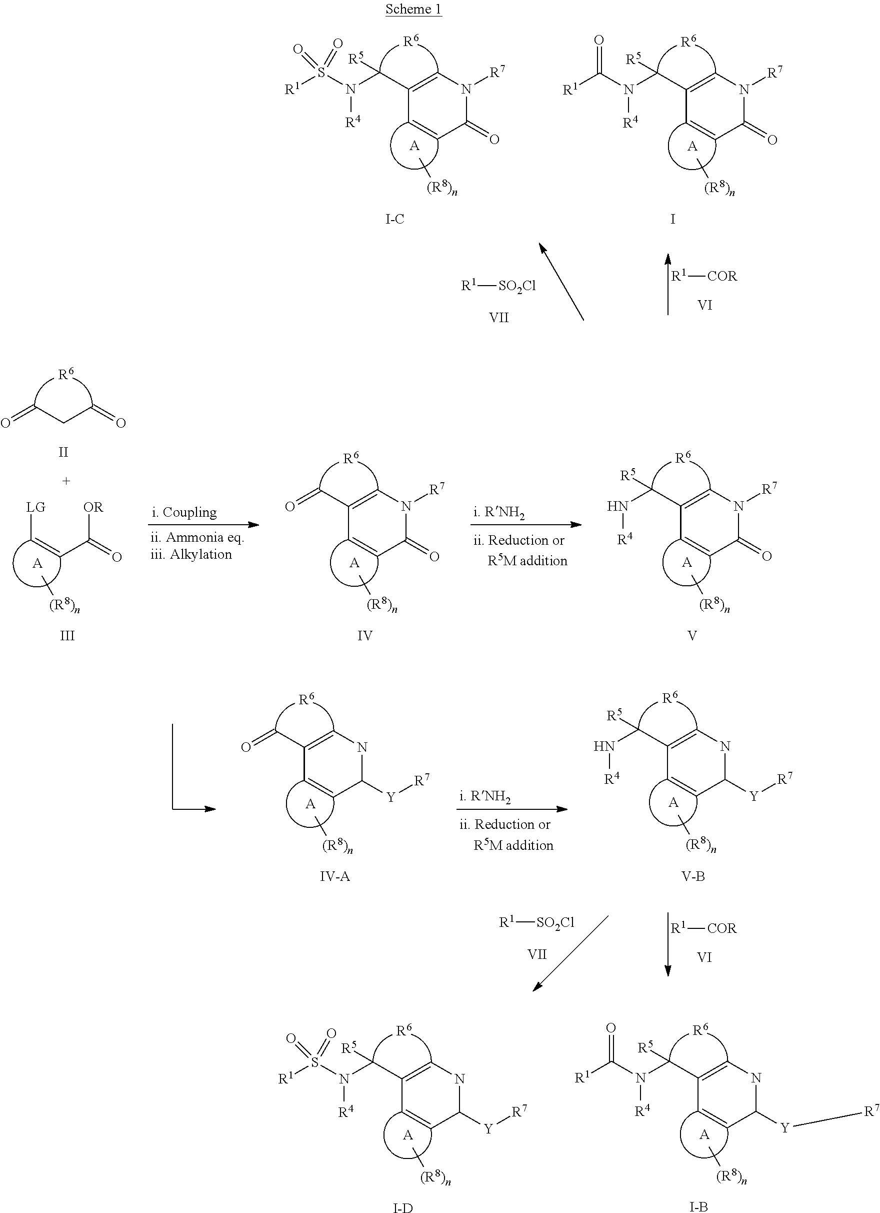

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

A compound of formula (I) can be prepared from commercially available or previously documented starting materials, for example, according to the synthetic methods outlined in Scheme 1.

Bi- or tri-cyclic ketones IV can be prepared from 1,3-diketones II and carboxylic acid derivatives III by a coupling reaction (when LG in III is a suitable leaving group, in non-limiting examples, a halogen, triflate, tosylate, mesylate, and so forth) in the presence of a metal catalyst such as, but not limited to, copper iodide, or by an aldol-type condensation (when III is β-ketoacid or β-ketoester), followed by reaction of the generated intermediates, either isolated or in situ, with ammonia or amines and then optionally by alkylation. In the latter case, O-alkylation provides ketone IV-A. Ketones IV and IV-A are condensed with amines and the resulting intermediate imines are reacted with a reducing agent, such as but not limited to sodium borohydride, or carbon-based nucleophiles, such as but not limited to a Grignard reagent or an alkyl/aryl lithium reagent to afford amines V, or V-B. In certain embodiments, the primary R′NH₂ amine can contain a chiral center which can be racemic, scalemic, or enantiopure, and can be used to influence the stereochemical outcome of the imine reduction or carbon-based nucleophile addition. The resulting secondary amine can be further reacted with an aldehyde and a reducing agent such as but not limited to sodium triacetoxyborohydride, and the R′ group can be removed to provide V, or V-B. Alternatively, IV and IV-A can be reacted with a primary sulfinamide to form a sulfinimine, which is subsequently reacted with a reducing agent, such as but not limited to sodium borohydride, or a carbon-based nucleophile, such as but not limited to a Grignard reagent or an alkyl/aryl lithium. In certain embodiments, the primary sulfinamide can be racemic, scalemic, or enantiopure, and can be used to influence the stereochemical outcome of the sulfinimine reduction. The resulting secondary sulfinamide can be further functionalized with an electrophile, such as but not limited to an alkyl halide, in the presence of base, such as but not limited to sodium hydride, and the sulfinamido group can be removed to provide V, or V-B. Functionalization of V or V-B with a variety of electrophiles, for example an activated carboxylic acid derivative VI, or a sulfonyl chloride VII, provides, respectively, I, I-B, I-C, or I-D.

The protocols incorporated elsewhere herein exemplify synthesis of representative compounds of the present disclosure. Analogous compounds can be synthesized in a similar fashion to those exemplified using the appropriately substituted intermediates and reagents.

### Methods

The disclosure provides a method of treating, ameliorating, and/or preventing hepatitis virus infection in a subject. In certain embodiments, the infection comprises hepatitis B virus (HBV) infection. In other embodiments, the infection comprises hepatitis D virus (HDV) infection. In yet other embodiments, the infection comprises HBV infection and HDV infection. In yet other embodiments, the method comprises administering to the subject in need thereof a therapeutically effective amount of at least one compound of the disclosure. In yet other embodiments, the at least one compound of the disclosure is the only antiviral agent administered to the subject. In yet other embodiments, the at least one compound is administered to the subject in a pharmaceutically acceptable composition. In yet other embodiments, the subject is further administered at least one additional agent useful for treating, ameliorating, and/or preventing the hepatitis infection. In yet other embodiments, the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator, such as checkpoint inhibitor (e.g., PD-L1 inhibitor); GalNAc-siRNA conjugate targeted against an HBV gene transcript; and therapeutic vaccine. In yet other embodiments, the subject is co-administered the at least one compound and the at least one additional agent. In yet other embodiments, the at least one compound and the at least one additional agent are coformulated.

The disclosure further provides a method of inhibiting expression and/or function of a viral capsid protein either directly or indirectly in a subject. In certain embodiments, the method comprises administering to the subject in need thereof a therapeutically effective amount of at least one compound of the disclosure. In other embodiments, the at least one compound is administered to the subject in a pharmaceutically acceptable composition. In yet other embodiments, the at least one compound of the disclosure is the only antiviral agent administered to the subject. In yet other embodiments, the subject is further administered at least one additional agent useful for treating, ameliorating, and/or preventing HBV infection. In yet other embodiments, the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator, such as checkpoint inhibitor (e.g., PD-L1 inhibitor); GalNAc-siRNA conjugate targeted against an HBV gene transcript; and therapeutic vaccine. In yet other embodiments, the subject is co-administered the at least one compound and the at least one additional agent. In yet other embodiments, the at least one compound and the at least one additional agent are coformulated.

In certain embodiments, the subject is a mammal. In other embodiments, the mammal is a human.

### Pharmaceutical Compositions and Formulations

The disclosure provides pharmaceutical compositions comprising at least one compound of the disclosure or a salt or solvate thereof, which are useful to practice methods of the disclosure. Such a pharmaceutical composition may consist of at least one compound of the disclosure or a salt or solvate thereof, in a form suitable for administration to a subject, or the pharmaceutical composition may comprise at least one compound of the disclosure or a salt or solvate thereof, and one or more pharmaceutically acceptable carriers, one or more additional ingredients, or any combinations of these. At least one compound of the disclosure may be present in the pharmaceutical composition in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 1,000 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the disclosure may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the disclosure may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the disclosure are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In certain embodiments, the compositions of the disclosure are formulated using one or more pharmaceutically acceptable excipients or carriers. In certain embodiments, the pharmaceutical compositions of the disclosure comprise a therapeutically effective amount of at least one compound of the disclosure and a pharmaceutically acceptable carrier.

Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

Compounds of the disclosure for administration may be in the range of from about 1 pg to about 7,500 mg, about 20 pg to about 7,000 mg, about 40 pg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the disclosure is from about 0.5 pg and about 5,000 mg. In some embodiments, a dose of a compound of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, and/or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the disclosure may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present disclosure also includes a multi-layer tablet comprising a layer providing for the delayed release of one or more compounds useful within the methods of the disclosure, and a further layer providing for the immediate release of one or more compounds useful within the methods of the disclosure. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the disclosure which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the disclosure may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically- or naturally derived.

### Buccal Administration

A pharmaceutical composition of the disclosure may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the disclosure includes additional modifications of these and other formulations not described herein, but which are known to those of skill in the art.

### Rectal Administration

A pharmaceutical composition of the disclosure may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the compositions and/or formulations of the present disclosure may be, but are not limited to, short-term, rapid-onset and/or rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present disclosure. However, they are in no way a limitation of the teachings or disclosure of the present disclosure as set forth herein.

### Examples

The disclosure is now described with reference to the following Examples. These Examples are provided for the purpose of illustration only, and the disclosure is not limited to these Examples, but rather encompasses all variations that are evident as a result of the teachings provided herein.

### Materials & Methods

The following procedures can be utilized in evaluating and selecting compounds that inhibit hepatitis B virus infection.

HepDE19 Assay with bDNA Quantitation of HBV rcDNA:

HepDE19 cell culture system is a HepG2 (human hepatocarcinoma) derived cell line that supports HBV DNA replication and cccDNA formation in a tetracycline (Tet)-regulated manner and produces HBV rcDNA and a detectable reporter molecule dependent on the production and maintenance of cccDNA (Guo, et al., 2007, J. Virol. 81:12472-12484).

HepDE19 (50,000 cells/well) were plated in 96-well collagen-coated tissue-culture treated microtiter plates in DMEM/F12 medium supplemented with 10% fetal bovine serum, 1% penicillin-streptomycin and 1 μg/mL tetracycline and incubated in a humidified incubator at 37° C. and 5% CO₂ overnight. Next day, the cells were switched to fresh medium without tetracycline and incubated for 4 hours at 37° C. and 5% CO₂. The cells were treated with fresh Tet-free medium with compounds at concentrations starting at 25 μM and a serial, ½ log, 8-point, titration series in duplicate. The final DMSO concentration in the assay was 0.5%. The plates were incubated for 7 days in a humidified incubator at 37° C. and 5% CO₂. Following a 7 day-incubation, the level of rcDNA present in the inhibitor-treated wells was measured using a Quantigene 2.0 bDNA assay kit (Affymetrix, Santa Clara, CA) with HBV specific custom probe set and manufacturers instructions. Concurrently, the effect of compounds on cell viability was assessed using replicate plates, plated at a density of 5,000 cells/well and incubated for 4 days, to determine the ATP content as a measure of cell viability using the cell-titer glo reagent (CTG; Promega Corporation, Madison, WI) as per manufacturer's instructions. The plates were read using a Victor luminescence plate reader (PerkinElmer Model 1420 Multilabel counter) and the relative luminescence units (RLU) data generated from each well was calculated as % inhibition of the untreated control wells and analyzed using XL-Fit module in Microsoft Excel to determine EC₅₀ and EC₉₀ (bDNA) and CC₅₀ (CTG) values using a 4-parameter curve fitting algorithm.

### LCMS Methods:

LCMS Method A: Waters Acquity UPLC system employing a Waters Acquity UPLC BEH C18, 1.7 μm, 50×2.1 mm column with an aqueous acetonitrile based solvent gradient of 2-98% CH₃CN/H₂O (0.05% TFA) over 9.5 mins. Flow rate=0.8 mL/min.

LCMS Method B: Waters Acquity UPLC system employing a Waters Acquity UPLC BEH C18, 1.7 μm, 50×2.1 mm column with an aqueous acetonitrile based solvent gradient of 2-98% CH₃CN/H₂O (0.05% TFA) over 1.0 mins. Flow rate=0.8 mL/min.

LCMS Method C: Shimadzu UFLC system employing an ACE UltraCore Super PhenylHexyl, 2.5 μm, 50×2.1 mm column with an aqueous acetonitrile based solvent gradient of 5-100% CH₃CN/H₂O (0.05% Formic acid) over 5.0 mins. Flow rate=1.0 mL/min.

LCMS Method D: Waters Acquity UPLC system employing a Waters Acquity UPLC BEH C18, 1.7 μm, 50×2.1 mm column with an aqueous acetonitrile based solvent gradient of 2-98% CH₃CN/H₂O (0.05% TFA) over 5.0 mins. Flow rate=0.8 mL/min.

LCMS Method E: Waters Acquity UPLC system employing a Waters Acquity UPLC BEH C18 2.1×50 mm; 1.7 μm, Mobile Phase-A: 0.05% FA in H₂O. Mobile phase-B: 0.05% FA in ACN; Gradient: T/% B: 0-5; 0.3-5; 2.5-95, 3.7-95, 4-5; 4.6-5; Flow rate: 0.6 mL/min; Column Temp: 40° C.

As described herein, “Enantiomer I” or “Diastereomer I” refers to the first enantiomer or diastereomer eluded from the chiral column under the specific chiral analytical conditions detailed for examples provided elsewhere herein; and “Enantiomer II” or “Diastereomer II” refers to the second enantiomer or diastereomer eluded from the chiral column under the specific chiral analytical conditions detailed for examples provided elsewhere herein. Such nomenclature does not imply or impart any particular relative and/or absolute configuration for these compounds.

### Example 1: Compounds

### 8-Fluoro-4,5-dihydropyrano[3,4-c]isoquinoline-1,6-dione (IVa)

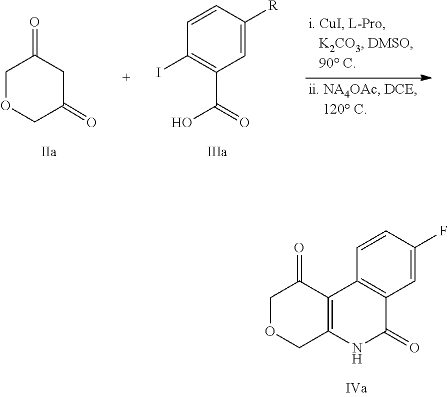

Step i: 5-Fluoro-2-iodo-benzoic acid (IIIa, 2.51 g, 9.44 mmol), tetrahydropyran-3,5-dione (IIa, 3.23 g, 28.31 mmol), copper (I) iodide (0.18 g, 0.94 mmol), L-proline (0.22 g, 1.89 mmol), and potassium dicarbonate (8.69 g, 37.74 mmol) were combined in a tube and evacuated and filled with nitrogen. Dry DMSO (30 mL) was added and the reaction mixture was purged with nitrogen, sealed, and stirred at room temperature for 10 min, and then at 90° C. for 2.5 h. The reaction mixture was allowed to cool to room temperature, diluted with 8 mL water, acidified with 2 M HCl to pH<2, and extracted with ethyl acetate (3×100 mL). The combined organic extracts were washed 3 times with water and once with brine, dried over sodium sulfate, and filtered. The solvent was evaporated under high vacuum to afford a crude product which was further dried under high vacuum overnight (when complete solidification occurred) and used in the next step without further purification.

Step ii: Crude 5-fluoro-2-(3-hydroxy-5-oxo-2H-pyran-4-yl)benzoic acid (2.38 g, 9.44 mmol) obtained in previous step and ammonium acetate (7.27 g, 94.37 mmol) were stirred in 1,2-dichloroethane (100 mL) at 120° C., in a sealed tube for 5 h. The reaction mixture was diluted with dichloromethane/methanol and adsorbed onto silica gel, then submitted to flash chromatography (silica gel, MeOH/DCM 0-10%). The desired product was further triturated with EtOAc/Hexanes to afford 8-fluoro-4,5-dihydropyrano[3,4-c]isoquinoline-1,6-dione (1.15 g, 52.3%). LCMS m/z found 234.1 [M+H]⁺; RT=0.77 min, (Method B); ¹H NMR (400 MHz, DMSO-d₆) δ 12.18 (s, 1H), 9.06 (dd, 1H), 7.86 (dd, 1H), 7.70 (ddd, 1H), 4.76 (s, 2H), 4.25 (s, 2H).

### 8-Fluoro-1-(methylamino)-1,5-dihydro-2H-pyrano[3,4-c]isoquinolin-6(4H)-one (Va)

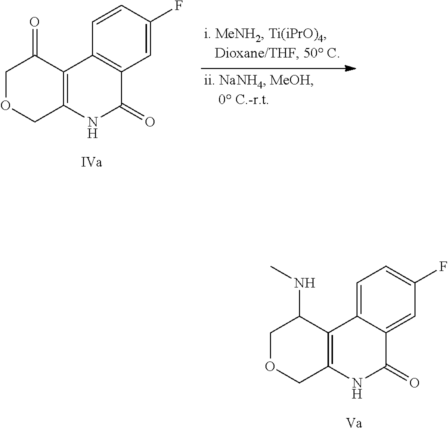 [SMILES: O=C1COCc2[nH]c(=O)c3cc(F)ccc3c21 ; CNC1COCc2[nH]c(=O)c3cc(F)ccc3c21]

Tetraisopropoxytitanium (1.04 mL, 3.43 mmol) was added to a mixture of 8-fluoro-4,5-dihydropyrano[3,4-c]isoquinoline-1,6-dione (IVa, 200.0 mg, 0.86 mmol) and a 2 M methylamine solution in THF (1.29 mL, 2.57 mmol) in 1,4-dioxane (9 mL). The mixture was stirred under nitrogen at 50° C. for 2 h. A small aliquot was removed and treated with sodium borohydride in methanol. LCMS analysis indicated complete conversion of starting material to product. The remaining reaction mixture was allowed to cool to room temperature, diluted with 3 mL anhydrous methanol and treated with sodium borohydride (64.9 mg, 1.72 mmol) at 0° C. After 5 min the cooling bath was removed, and the reaction mixture was stirred for 1 h. The reaction was quenched by addition of brine (1.5 mL), diluted with 20 mL of ethyl acetate, and stirred for 15 min, then filtered through CELITE®. The filter cake was washed with additional 25 mL of ethyl acetate, and the combined organic solutions were dried over sodium sulfate, filtered, and the solvent was evaporated under reduced pressure. The residue was adsorbed onto silica gel and the solvent evaporated. The product was isolated by flash-chromatography (silica gel, MeOH/DCM 0-10% gradient). LCMS: m/z found 249.2 [M+H]⁺; RT=0.49 min, (Method B); ¹H NMR (400 MHz, CDCl₃) δ 8.00-7.92 (m, 1H), 7.68 (dd, 1H), 7.42 (dddd, 1H), 4.60 (d, 1H), 4.53-4.44 (m, 1H), 4.36 (dd, 1H), 3.60 (dd, 1H), 3.55 (dt, 1H), 2.55 (d, 3H).

### N-(8-Fluoro-6-oxo-1,4,5,6-tetrahydro-2H-pyrano[3,4-c]isoquinolin-1-yl)-N-methyl-1H-indole-2-carboxamide (Compounds 1 and 2)

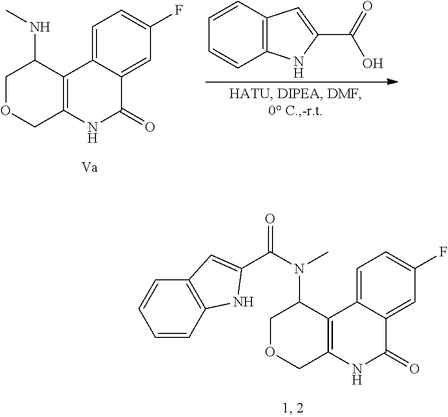

To a stirred solution of 0.11 g (0.66 mmol) of 1H-indole-2-carboxylic acid (VIa) in 3 mL of DMF at room temperature were added 0.32 mL (1.81 mmol) of DIPEA followed by 0.28 g (0.72 mmol) of HATU and stirring was continued for 15 min. To this mixture, 0.15 g (0.60 mmol) of 8-fluoro-1-(methylamino)-1,5-dihydro-2H-pyrano[3,4-c]isoquinolin-6(4H)-one (Va) was added at room temperature and stirring was continued for 6 hours. The reaction mixture was diluted with ice-cold water (20 mL) and stirred for a further 15 min at room temperature. The formed solid was collected by filtration, washed with water and dried under vacuum. The solid product was triturated with ethyl acetate (5 mL), collected by filtration and dried under vacuum to afford 0.16 g (0.40 mmol, 66%) of N-(8-fluoro-6-oxo-1,4,5,6-tetrahydro-2H-pyrano[3,4-c]isoquinolin-1-yl)-N-methyl-1H-indole-2-carboxamide. The enantiomers were subsequently separated by preparative SFC: Method isocratic, Mobile phase 2-propanol:CO₂—35:65. Column: Chiralpak IC (30×250 mm), 5μ, flow rate: 100 g/min.

Enantiomer I (Compound 1): LCMS: m/z found 392.3 [M+H]⁺, RT=3.77 min, (Method A); ¹H NMR (400 MHz, DMSO-d₆): δ 11.68 (br s, 2H), 7.92-7.88 (m, 1H), 7.65-7.58 (m, 2H), 7.54-7.43 (m, 2H), 7.22-7.18 (m, 1H), 7.06-7.02 (m, 1H), 6.90 (s, 1H), 5.76-5.75 (m, 1H), 4.63 (d, 1H), 4.48 (d, 1H), 4.16 (d, 1H), 4.06 (d, 1H), 3.15 (s, 3H); Chiral analytical SFC: RT=4.09 min, Column: CHIRALPAK IC-3 (4.6×150 mm) 3 μm, 40% of (0.5% DEA in Methanol), Flow rate: 3.0 g/min.

Enantiomer II (Compound 2): LCMS: m/z found 392.2 [M+H]⁺, RT=3.77 min, (Method A); ¹H NMR (400 MHz, DMSO-d₆): δ 11.68 (br s, 2H), 7.92-7.88 (m, 1H), 7.65-7.58 (m, 2H), 7.54-7.43 (m, 2H), 7.22-7.18 (m, 1H), 7.06-7.02 (m, 1H), 6.90 (s, 1H), 5.76-5.75 (m, 1H), 4.63 (d, 1H), 4.48 (d, 1H), 4.16 (d, 1H), 4.06 (d, 1H), 3.15 (s, 3H); Chiral analytical SFC: RT=5.58 min, Column: CHIRALPAK IC-3 (4.6×150 mm) 3 μm, 40% of (0.5% DEA in Methanol), Flow rate: 3.0 g/min.

### (2S)-N-(8-Fluoro-6-oxo-1,4,5,6-tetrahydro-2H-pyrano[3,4-c]isoquinolin-1-yl)-N-methylindoline-2-carboxamide (Compounds 3 and 4)

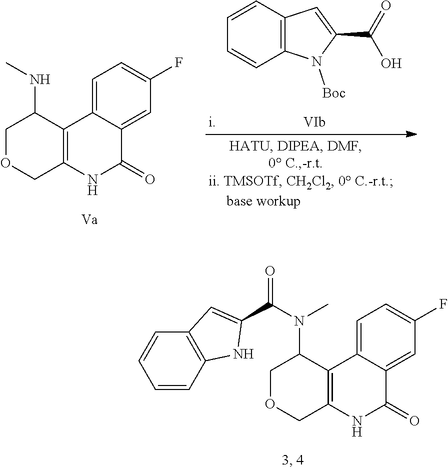

Step i: To a stirred solution of 0.19 g (0.76 mmol) of (S)-1-(tert-butoxycarbonyl)indoline-2-carboxylic acid (VIb) in 2 mL of DMF at room temperature were added 0.40 mL (0.76 mmol) of DIPEA followed by 0.35 g (0.91 mmol) of HATU and the mixture was stirred for 15 min. To this mixture was added 0.20 g (0.76 mmol) of 8-fluoro-1-(methylamino)-1,5-dihydro-2H-pyrano[3,4-c]isoquinolin-6(4H)-one (Va) at room temperature and stirring was continued for 16 h. The mixture was then diluted with ice-cold water (30 mL) and stirred at room temperature for a further 2 h. The resulting precipitate was collected by filtration, washed with water and dried under vacuum. The solid product triturated with n-pentane (15 mL) at room temperature for 15 min, collected by filtration and dried under vacuum to afford 275 mg (0.55 mmol, 73%) of tert-butyl(2S)-2-((8-fluoro-6-oxo-1,4,5,6-tetrahydro-2H-pyrano[3,4-c]isoquinolin-1-yl)(methyl)carbamoyl)indoline-1-carboxylate, which was taken as such for next step: LCMS: m/z found 494.29 [M+H]⁺, 2 diastereoisomers: RT=2.05 min and 2.07 min (Method A).

Step ii: To a stirred solution of 0.27 g (0.54 mmol) of tert-butyl (2S)-2-((8-fluoro-6-oxo-1,4,5,6-tetrahydro-2H-pyrano[3,4-c]isoquinolin-1-yl)(methyl)carbamoyl)indoline-1-carboxylate in 5.4 mL of dichloromethane at 0° C. under a nitrogen atmosphere was added 0.24 g (1.09 mmol) of trimethylsilyl trifluoromethanesulfonate (TMSOTf) and the mixture was stirred at room temperature for 1 h. The mixture was then concentrated under reduced pressure and the residue was basified with saturated NaHCO₃ solution (15 mL). The solid precipitate was collected by filtration and dried under high vacuum to afford 0.21 g (0.53 mmol, 97%) of (2S)-N-(8-fluoro-6-oxo-1,4,5,6-tetrahydro-2H-pyrano[3,4-c]isoquinolin-1-yl)-N-methylindoline-2-carboxamide. The diastereoisomers were subsequently separated by preparative SFC: Method isocratic, Mobile phase 2-propanol:CO₂—20:80. Column: Daicel DCPACK (30×250 mm), 5 μm, flow rate: 90 g/min.

Diastereoisomer I (Compound 3): LCMS: m/z found 394.3 [M+H]⁺, RT=2.64 min, (Method A); ¹H NMR (400 MHz, DMSO-d₆): δ 11.58 (br s, 1H), 7.89 (d, 1H), 7.57-7.52 (m, 1H), 7.28-7.25 (m, 1H), 7.01-6.93 (m, 2H), 6.61-6.56 (m, 2H), 5.74 (s, 1H), 5.54 (s, 1H), 4.74-4.70 (m, 1H), 4.59 (d, 1H), 4.43 (d, 1H), 4.03-3.93 (m, 2H), 3.31-3.25 (m, 1H), 2.90-2.85 (m, 1H), 2.84 (s, 3H); Chiral analytical SFC: RT=4.44 min, Column: DC PAK SFC-B (4.6×150 mm) 5 μm, 20% Methanol, Flow rate: 3.0 g/min.

Diastereoisomer II (Compound 4): LCMS: m/z found 394.2 [M+H]⁺, RT=2.42 min, (Method A); ¹H NMR (400 MHz, DMSO-d₆): δ 11.58 (br s, 1H), 7.91-7.87 (m, 1H), 7.62-7.56 (m, 1H), 7.46-7.42 (m, 1H), 7.01 (d, 1H), 6.963-6.92 (m, 1H), 6.59-6.54 (m, 2H), 5.74 (s, 1H), 5.59 (s, 1H), 4.69-4.64 (m, 1H), 4.58 (d, 1H), 4.43 (d, 1H), 3.99-3.89 (m, 2H), 3.32-3.30 (m, 1H), 3.19-3.13 (m, 1H), 2.85 (s, 3H); Chiral analytical SFC: RT=6.33 min, Column: DC PAK SFC-B (4.6×150 mm) 5 μm, 20% Methanol, Flow rate: 3.0 g/min.

### 4-Fluoro-N-(8-fluoro-6-oxo-1,4,5,6-tetrahydro-2H-pyrano[3,4-c]isoquinolin-1-yl)-N-methyl-1H-indole-2-carboxamide: (Compounds 19 and 20)

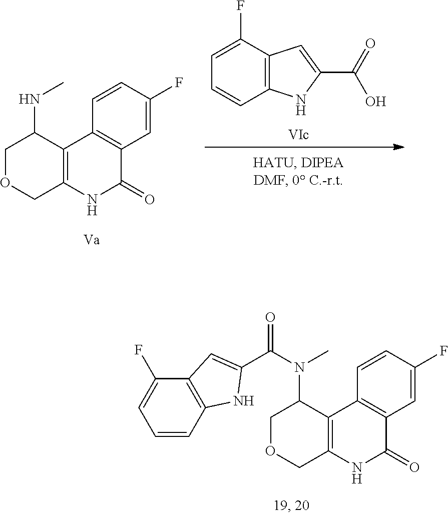

Racemic 4-fluoro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from racemic 8-fluoro-1-(methylamino)-1,5-dihydro-2H-pyrano[3,4-c]isoquinolin-6(4H)-one (Va) and 4-fluoro-1H-indole-2-carboxylic acid (VIc). The enantiomers were subsequently separated by preparative SFC: Method isocratic, Mobile phase methanol:CO₂—40:60. Column: Chiralpak IC (30×250 mm), 5μ, flow rate: 110 g/min.

Enantiomer I (Compound 19): LCMS: m/z found 410.2 [M+H]⁺, RT=3.95 min, (Method A); ¹H NMR (400 MHz, DMSO-d₆): δ 12.1 (br s, 2H), 7.91-7.88 (m, 1H), 7.65-7.62 (m, 1H), 7.54-7.50 (m, 1H), 7.29 (d, 1H), 7.21-7.16 (m, 1H) 6.93 (s, 1H), 6.84-6.79 (t, 1H), 5.75 (br s, 1H), 4.61 (d, 1H), 4.46 (d, 1H), 4.15 (d, 1H), 4.03 (d, 1H), 3.15 (s, 3H); Chiral analytical SFC: RT=2.82 min, Column: CHIRALPAK IC-3 (4.6×150 mm) 3 μm, 40% of Methanol, Flow rate: 3.0 g/min.

Enantiomer II (Compound 20): LCMS: m/z found 410.2 [M+H]⁺, RT=3.95 min, (Method A); ¹H NMR (400 MHz, DMSO-d₆): δ 12.1 (br s, 2H), 7.91-7.88 (m, 1H), 7.65-7.62 (m, 1H), 7.54-7.50 (m, 1H), 7.29 (d, 1H), 7.21-7.16 (m, 1H) 6.93 (s, 1H), 6.84-6.79 (t, 1H), 5.75 (br s, 1H), 4.61 (d, 1H), 4.46 (d, 1H), 4.15 (d, 1H), 4.03 (d, 1H), 3.15 (s, 3H); Chiral analytical SFC: RT=3.21 min, Column: CHIRALPAK IC-3 (4.6×150 mm) 3 μm, 40% of Methanol, Flow rate: 3.0 g/min.

### 5-Fluoro-N-(8-fluoro-6-oxo-1,4,5,6-tetrahydro-2H-pyrano[3,4-c]isoquinolin-1-yl)-N-methyl-1H-indole-2-carboxamide (Compounds 29 and 30)

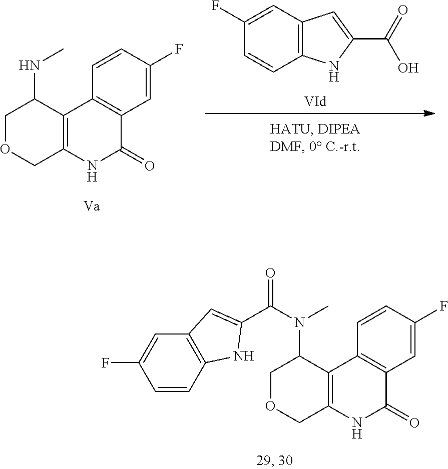

Racemic 5-fluoro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from racemic 8-fluoro-1-(methylamino)-1,5-dihydro-2H-pyrano[3,4-c]isoquinolin-6(4H)-one (Va) and 5-fluoro-1H-indole-2-carboxylic acid (VId). The enantiomers were subsequently separated by preparative SFC: Method isocratic, Mobile phase methanol:CO₂—35:65. Column: Chiralpak IC (30×250 mm), 5 μm, flow rate: 110 g/min.

Enantiomer I (Compound 29): LCMS: m/z found 410.2 [M+H]⁺, RT=3.90 min, (Method A); ¹H NMR (400 MHz, DMSO-d₆): δ 11.79 (br s 2H), 7.90 (dd, 1H), 7.62-7.59 (m, 1H), 7.52-7.44 (m, 2H), 7.35 (dd, 1H), 7.09-7.04 (m, 1H), 6.88 (s, 1H), 5.74 (br s, 1H), 4.62 (d, 1H), 4.46 (d, 1H), 4.16 (d, 1H), 4.04 (d, 1H), 3.13 (s, 3H); Chiral analytical SFC: RT=3.12 min, Column: CHIRALPAK IC-3 (4.6×150 mm) 3 μm, 40% of Methanol, Flow rate: 3.0 g/min.

Enantiomer II (Compound 30): LCMS: m/z found 410.2 [M+H]⁺, RT=3.90 min, (Method A); ¹H NMR (400 MHz, DMSO-d₆): δ 11.79 (br s 2H), 7.90 (dd, 1H), 7.61-7.57 (m, 1H), 7.51-7.44 (m, 2H), 7.35 (dd, 1H), 7.09-7.04 (m, 1H), 6.87 (s, 1H), 5.74 (br s, 1H), 4.62 (d, 1H), 4.46 (d, 1H), 4.16 (d, 1H), 4.04 (d, 1H), 3.13 (s, 3H); Chiral analytical SFC: RT=3.75 min, Column: CHIRALPAK IC-3 (4.6×150 mm) 3 μm, 40% of Methanol, Flow rate: 3.0 g/min.

### 6-Fluoro-N-(8-fluoro-6-oxo-1,4,5,6-tetrahydro-2H-pyrano[3,4-c]isoquinolin-1-yl)-N-methyl-1H-indole-2-carboxamide (Compounds 31 and 32)

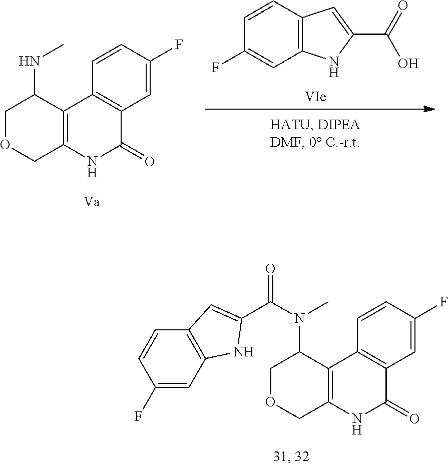

Racemic 6-fluoro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from racemic 8-fluoro-1-(methylamino)-1,5-dihydro-2H-pyrano[3,4-c]isoquinolin-6(4H)-one (Va) and 6-fluoro-1H-indole-2-carboxylic acid (VIe). The enantiomers were subsequently separated by preparative SFC: Method isocratic, Mobile phase methanol:CO₂—35:65. Column: Chiralpak IC (30×250 mm), 5 μm, flow rate: 110 g/min.

Enantiomer I (Compound 31): LCMS: m/z found 410.2 [M+H]⁺, RT=3.94 min, (Method A); ¹H NMR (400 MHz, DMSO-d₆): δ 11.76 (br s 2H), 7.84 (dd, 1H), 7.63-7.55 (m, 2H), 7.50-7.46 (m, 1H), 7.18 (dd, 1H), 6.93-6.89 (m, 2H), 6.88 (s, 1H), 5.74 (br s, 1H), 4.60 (d, 1H), 4.46 (d, 1H), 4.14 (d, 1H), 4.03 (d, 1H), 3.14 (s, 3H); Chiral analytical SFC: RT=3.16 min, Column: CHIRALPAK IC-3 (4.6×150 mm) 3 μm, 40% of Methanol, Flow rate: 3.0 g/min.

Enantiomer II (Compound 32): LCMS: m/z found 410.2 [M+H]⁺, RT=3.94 min, (Method A); ¹H NMR (400 MHz, DMSO-d₆): δ 11.76 (br s 2H), 7.84 (dd, 1H), 7.63-7.55 (m, 2H), 7.50-7.46 (m, 1H), 7.18 (dd, 1H), 6.93-6.89 (m, 2H), 6.88 (s, 1H), 5.74 (br s, 1H), 4.60 (d, 1H), 4.46 (d, 1H), 4.14 (d, 1H), 4.03 (d, 1H), 3.14 (s, 3H); Chiral analytical SFC: RT=3.85 min, Column: CHIRALPAK IC-3 (4.6×150 mm) 3 μm, 40% of Methanol, Flow rate: 3.0 g/min.

### 7-Fluoro-N-(8-fluoro-6-oxo-1,4,5,6-tetrahydro-2H-pyrano[3,4-c]isoquinolin-1-yl)-N-methyl-1H-indole-2-carboxamide (Compounds 33 and 34)

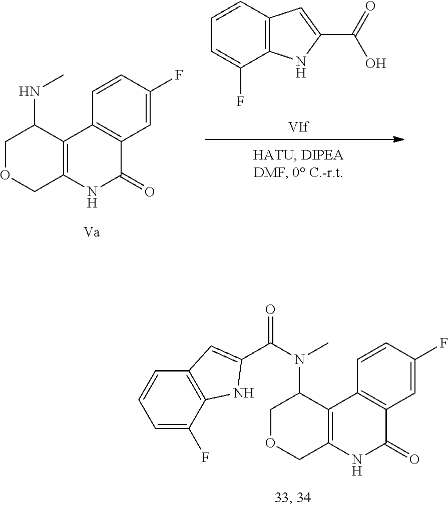

Racemic 7-fluoro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from racemic 8-fluoro-1-(methylamino)-1,5-dihydro-2H-pyrano[3,4-c]isoquinolin-6(4H)-one (Va) and 7-fluoro-1H-indole-2-carboxylic acid (VIf). The enantiomers were subsequently separated by preparative SFC: Method isocratic, Mobile phase methanol:CO₂—40:60. Column: Chiralpak IC (30×250 mm), 5 μm, flow rate: 110 g/min.

Enantiomer I (Compound 33): LCMS: m/z found 410.2 [M+H]⁺, RT=3.90 min, (Method A); ¹H NMR (400 MHz, DMSO-d₆): δ 12.1 (br s, 2H), 7.91-7.89 (m, 1H), 7.66-7.54 (m, 2H), 7.42-7.40 (m, 1H), 7.04-6.99 (m, 2H) 6.91 (d, 1H), 5.74 (br s, 1H), 4.60 (d, 1H), 4.46 (d, 1H), 4.16 (d, 1H), 4.04 (d, 1H), 3.08 (s, 3H); Chiral analytical SFC: RT=3.59 min, Column: CHIRALPAK IC-3 (4.6×150 mm) 3 μm, 40% of Methanol, Flow rate: 3.0 g/min.

Enantiomer II (Compound 34): LCMS: m/z found 410.2 [M+H]⁺, RT=3.90 min, (Method A); ¹H NMR (400 MHz, DMSO-d₆): δ 12.1 (br s, 2H), 7.91-7.89 (m, 1H), 7.66-7.54 (m, 2H), 7.42-7.40 (m, 1H), 7.04-6.99 (m, 2H) 6.91 (d, 1H), 5.74 (br s, 1H), 4.60 (d, 1H), 4.46 (d, 1H), 4.16 (d, 1H), 4.04 (d, 1H), 3.08 (s, 3H); Chiral analytical SFC: RT=5.00 min, Column: CHIRALPAK IC-3 (4.6×150 mm) 3 μm, 40% of Methanol, Flow rate: 3.0 g/min.

### 4,6-Difluoro-N-(8-fluoro-6-oxo-1,4,5,6-tetrahydro-2H-pyrano[3,4-c]isoquinolin-1-yl)-N-methyl-1H-indole-2-carboxamide (Compounds 35 and 36)

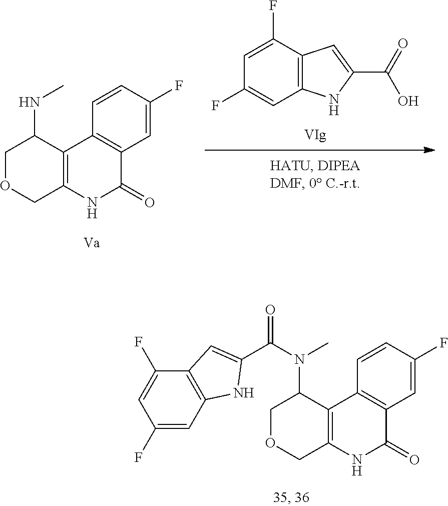

Racemic 4,6-difluoro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from racemic 8-fluoro-1-(methylamino)-1,5-dihydro-2H-pyrano[3,4-c]isoquinolin-6(4H)-one (Va) and 4,6-difluoro-1H-indole-2-carboxylic acid (VIg). The enantiomers were subsequently separated by preparative SFC: Method isocratic, Mobile phase methanol:CO₂—40:60. Column: Chiralcel-OD-H (30×250 mm), 5 μm, flow rate: 100 g/min.

Enantiomer I (Compound 35): LCMS: m/z found 428.2 [M+H]⁺, RT=4.20 min, (Method A); ¹H NMR (400 MHz, DMSO-d₆): δ 11.88 (br s 2H), 7.89 (dd, 1H), 7.63-7.59 (m, 1H), 7.52-7.48 (m, 1H), 7.07 (dd, 1H), 6.97 (s, 1H), 6.91-6.86 (m, 1H), 5.75 (br s, 1H), 4.62 (d, 1H), 4.46 (d, 1H), 4.16 (d, 1H), 4.04 (d, 1H), 3.15 (s, 3H); Chiral analytical SFC: RT=2.35 min, Column: Chiralcel OD-3 (4.6×150 mm) 3 μm, 40% of Methanol, Flow rate: 3.0 g/min.

Enantiomer II (Compound 36): LCMS: m/z found 428.2 [M+H]⁺, RT=4.20 min, (Method A); ¹H NMR (400 MHz, DMSO-d₆): δ 11.88 (br s 2H), 7.89 (dd, 1H), 7.63-7.59 (m, 1H), 7.52-7.48 (m, 1H), 7.07 (dd, 1H), 6.97 (s, 1H), 6.91-6.86 (m, 1H), 5.75 (br s, 1H), 4.62 (d, 1H), 4.46 (d, 1H), 4.16 (d, 1H), 4.04 (d, 1H), 3.15 (s, 3H); Chiral analytical SFC: RT=2.84 min, Column: Chiralcel OD-3 (4.6×150 mm) 3 μm, 40% of Methanol, Flow rate: 3.0 g/min.

### 4,5-Difluoro-N-(8-fluoro-6-oxo-1,4,5,6-tetrahydro-2H-pyrano[3,4-c]isoquinolin-1-yl)-N-methyl-1H-indole-2-carboxamide (Compounds 37 and 38)

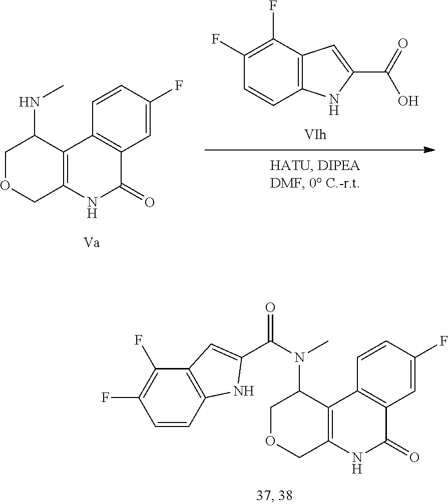

Racemic 4,5-difluoro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from racemic 8-fluoro-1-(methylamino)-1,5-dihydro-2H-pyrano[3,4-c]isoquinolin-6(4H)-one (Va) and 4,5-difluoro-1H-indole-2-carboxylic acid (VIh). The enantiomers were subsequently separated by preparative SFC: Method isocratic, Mobile phase methanol:CO₂—35:65. Column: Chiralpak AS-H (30×250 mm), 5 μm, flow rate: 120 g/min.

Enantiomer I (Compound 37): LCMS: m/z found 428.2 [M+H]⁺, RT=4.12 min, (Method A); ¹H NMR (400 MHz, DMSO-d₆): δ 11.91 (br s, 2H), 7.90-7.87 (m, 1H), 7.63-7.58 (m, 1H), 7.52-7.48 (m, 1H), 7.26-7.20 (m, 2H), 6.99 (s, 1H), 5.74 (s, 1H), 4.61 (d, 1H), 4.47 (d, 1H), 4.16 (d, 1H), 4.05 (d, 1H), 3.14 (s, 3H); Chiral analytical SFC: RT=3.75 min, Column: Chiralpak AS-3 (4.6×150 mm) 3 μm, 30% of Methanol, Flow rate: 3.0 g/min.

Enantiomer II (Compound 38): LCMS: m/z found 428.2 [M+H]⁺, RT=4.12 min, (Method A); ¹H NMR (400 MHz, DMSO-d₆): δ 11.91 (br s, 2H), 7.90-7.87 (m, 1H), 7.63-7.58 (m, 1H), 7.52-7.48 (m, 1H), 7.26-7.20 (m, 2H), 6.99 (s, 1H), 5.74 (s, 1H), 4.61 (d, 1H), 4.47 (d, 1H), 4.16 (d, 1H), 4.05 (d, 1H), 3.14 (s, 3H); Chiral analytical SFC: RT=5.62 min, Column: Chiralpak AS-3 (4.6×150 mm) 3 μm, 30% of Methanol, Flow rate: 3.0 g/min.

### 5,6-Difluoro-N-(8-fluoro-6-oxo-1,4,5,6-tetrahydro-2H-pyrano[3,4-c]isoquinolin-1-yl)-N-methyl-1H-indole-2-carboxamide (Compounds 49 and 50)

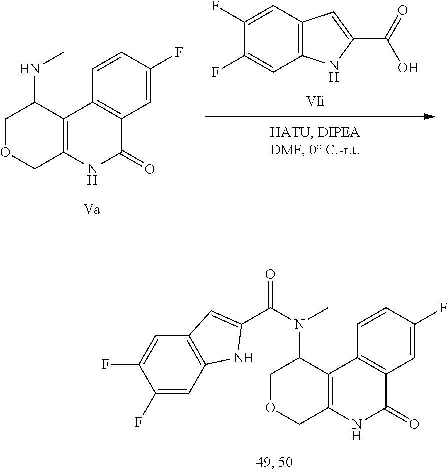

Racemic 5,6-difluoro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from racemic 8-fluoro-1-(methylamino)-1,5-dihydro-2H-pyrano[3,4-c]isoquinolin-6(4H)-one (Va) and 5,6-difluoro-1H-indole-2-carboxylic acid (VIi). The enantiomers were subsequently separated by preparative SFC: Method isocratic, Mobile phase methanol:CO₂—40:60. Column: Chiralcel-OD-H (30×250 mm), 5 μm, flow rate: 100 g/min.

Enantiomer I (Compound 49): LCMS: m/z found 428.2 [M+H]⁺, RT=4.12 min, (Method A); ¹H NMR (400 MHz, DMSO-d₆): δ 11.80-11.65 (br s, 2H), 7.89-7.86 (m, 1H), 7.61-7.56 (m, 2H), 7.49-7.45 (m, 1H), 7.40-7.36 (m, 1H), 6.91 (s, 1H), 5.73 (s, 1H), 4.60 (d, 1H), 4.46 (d, 1H), 4.15 (d, 1H), 4.03 (d, 1H), 3.16 (s, 3H); Chiral analytical SFC: RT=3.79 min, Column: Chiralcel OD-3 (4.6×150 mm) 3 μm, 30% of Methanol, Flow rate: 3.0 g/min.

Enantiomer II (Compound 50): LCMS: m/z found 428.2 [M+H]⁺, RT=4.62 min, (Method A); ¹H NMR (400 MHz, DMSO-d₆): δ 11.89 (s, 1H), 11.61 (br s, 1H), 7.92-7.89 (m, 1H), 7.66-7.59 (m, 2H), 7.52-7.49 (m, 1H), 7.40-7.36 (m, 1H), 6.92 (s, 1H), 5.74 (s, 1H), 4.64 (d, 1H), 4.48 (d, 1H), 4.16 (d, 1H), 4.05 (d, 1H), 3.13 (s, 3H); Chiral analytical SFC: RT=5.62 min, Column: Chiralcel OD-3 (4.6×150 mm) 3 μm, 30% of Methanol, Flow rate: 3.0 g/min.

### 4-Bromo-3,5-difluoro-N-(8-fluoro-6-oxo-2,4,5,6-tetrahydro-1H-pyrano[3,4-c]isoquinolin-1-yl)-N-methylbenzamide (Compounds 166 and 167)

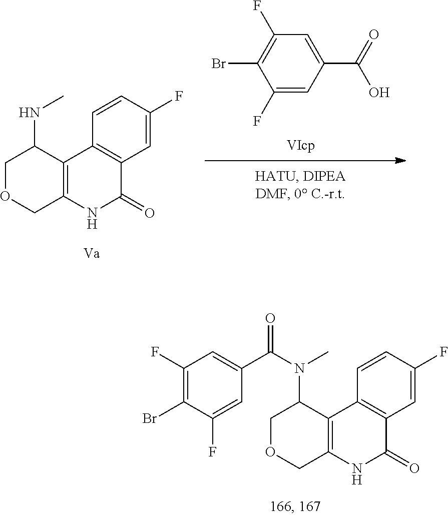 [SMILES: CNC1COCc2[nH]c(=O)c3cc(F)ccc3c21 ; O=C(O)c1cc(F)c(Br)c(F)c1 ; "VIcp" ; CN(C(=O)c1cc(F)c(Br)c(F)c1)C1COCc2[nH]c(=O)c3cc(F)ccc3c21]

Racemic 4-bromo-3,5-difluoro-N-(8-fluoro-6-oxo-2,4,5,6-tetrahydro-1H-pyrano[3,4-c]isoquinolin-1-yl)-N-methylbenzamide was synthesized in an analogous manner as described above from racemic 8-fluoro-1-(methylamino)-1,5-dihydro-2H-pyrano[3,4-c]isoquinolin-6(4H)-one (Va) and 4-bromo-3,5-difluorobenzoic acid (VIcp). The enantiomers were subsequently separated by preparative SFC: Method isocratic, Mobile phase (MeOH:MeCN (1:1)):CO₂—50:50. Column: Chiralpak-IG (30×250 mm), 5 μm, flow rate: 100 g/min.

Enantiomer I (Compound 166): LCMS: m/z found 467.1 [M+H]⁺, RT=5.76 min, (Method A); ¹H NMR (400 MHz, DMSO-d₆): δ 11.51 (s, 1H), 7.90 (dd, 1H), 7.75 (dt, 1H), 7.61-7.57 (m, 1H), 7.40 (d, 2H), 5.65 (s, 1H), 4.60 (d, 1H), 4.43 (d, 1H), 4.27 (d, 1H), 4.03-3.99 (m, 1H), 2.67 (s, 3H); Chiral analytical SFC: RT=1.21 min, Column: Chiralpak IG-3 (4.6×150 mm) 3 μm, 50% (MeOH:MeCN (1:1)), Flow rate: 3.0 g/min.

Enantiomer II (Compound 167): LCMS: m/z found 467.1 [M+H]⁺, RT=5.76 min, (Method A); ¹H NMR (400 MHz, DMSO-d₆): δ 11.51 (s, 1H), 7.90 (dd, 1H), 7.75 (dt, 1H), 7.61-7.57 (m, 1H), 7.40 (d, 2H), 5.65 (s, 1H), 4.60 (d, 1H), 4.43 (d, 1H), 4.27 (d, 1H), 4.03-3.99 (m, 1H), 2.67 (s, 3H); Chiral analytical SFC: RT=2.97 min, Column: Chiralpak IG-3 (4.6×150 mm) 3 μm, 50% (MeOH:MeCN (1:1)), Flow rate: 3.0 g/min.

### 4-Chloro-3,5-difluoro-N-(8-fluoro-6-oxo-2,4,5,6-tetrahydro-1H-pyrano[3,4-c]isoquinolin-1-yl)-N-methylbenzamide (Compounds 174 and 175)

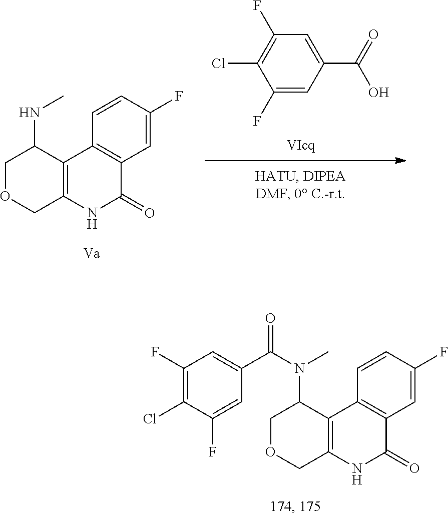 [SMILES: CNC1COCc2[nH]c(=O)c3cc(F)ccc3c21 ; O=C(O)c1cc(F)c(Cl)c(F)c1 ; "VIcq" ; CN(C(=O)c1cc(F)c(Cl)c(F)c1)C1COCc2[nH]c(=O)c3cc(F)ccc3c21]

Racemic 4-chloro-3,5-difluoro-N-(8-fluoro-6-oxo-2,4,5,6-tetrahydro-1H-pyrano[3,4-c]isoquinolin-1-yl)-N-methylbenzamide was synthesized in an analogous manner as described above from racemic 8-fluoro-1-(methylamino)-1,5-dihydro-2H-pyrano[3,4-c]isoquinolin-6(4H)-one (Va) and 4-chloro-3,5-difluorobenzoic acid (VIcq). The enantiomers were subsequently separated by preparative SFC: Method isocratic, Mobile phase (MeOH:MeCN (1:1)):CO₂—50:50. Column: Chiralpak-IG (30×250 mm), 5 μm, flow rate: 100 g/min.

Enantiomer I (Compound 174): LCMS: m/z found 423.3 [M+H]⁺, RT=6.29 min, (Method A); ¹H NMR (400 MHz, DMSO-d₆): δ 11.58 (s, 1H), 7.91-7.88 (dd, 1H), 7.77-7.72 (m, 1H), 7.61-7.57 (m, 1H), 7.47-7.33 (m, 2H), 5.65 (s, 1H), 4.58 (d, 1H), 4.42 (d, 1H), 4.27 (d, 1H), 4.03-3.99 (m, 1H), 2.67 (s, 3H); Chiral analytical SFC: RT=1.1 min, Column: Chiralpak IG-3 (4.6×150 mm) 3 μm, 50% (MeOH:MeCN (1:1)), Flow rate: 3.0 g/min.

Enantiomer II (Compound 175): LCMS: m/z found 423.3 [M+H]⁺, RT=6.29 min, (Method A); ¹H NMR (400 MHz, DMSO-d₆): δ 11.58 (s, 1H), 7.91-7.88 (dd, 1H), 7.77-7.72 (m, 1H), 7.61-7.57 (m, 1H), 7.47-7.33 (m, 2H), 5.65 (s, 1H), 4.58 (d, 1H), 4.42 (d, 1H), 4.27 (d, 1H), 4.03-3.99 (m, 1H), 2.67 (s, 3H); Chiral analytical SFC: RT=2.41 min, Column: Chiralpak IG-3 (4.6×150 mm) 3 μm, 50% (MeOH:MeCN (1:1)), Flow rate: 3.0 g/min.

### 4-(Difluoromethyl)-3,5-difluoro-N-(8-fluoro-6-oxo-1,4,5,6-tetrahydro-2H-pyrano[3,4-c]isoquinolin-1-yl)-N-methylbenzamide (Compounds 178 and 179)

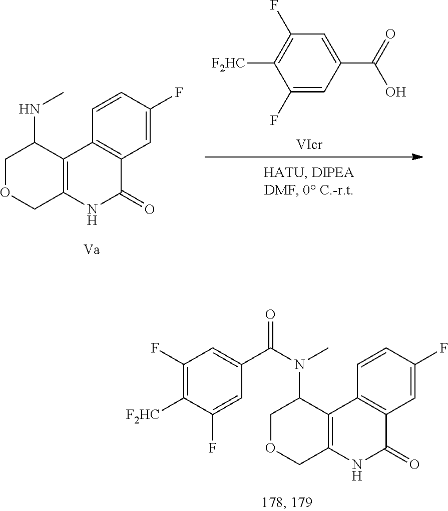

Racemic 4-(difluoromethyl)-3,5-difluoro-N-(8-fluoro-6-oxo-1,4,5,6-tetrahydro-2H-pyrano[3,4-c]isoquinolin-1-yl)-N-methylbenzamide was synthesized in an analogous manner as described above from racemic 8-fluoro-1-(methylamino)-1,5-dihydro-2H-pyrano[3,4-c]isoquinolin-6(4H)-one (Va) and 4-(difluoromethyl)-3,5-difluorobenzoic acid (VIcr). The enantiomers were subsequently separated by preparative SFC: Method isocratic, Mobile phase EtOH:CO₂—30:70. Column: Chiralpak-IA (30×250 mm), 5 μm, flow rate: 100 g/min.

Enantiomer I (Compound 178): LCMS: m/z found 439.3 [M+H]⁺, RT=6.07 min, (Method A); ¹H NMR (400 MHz, DMSO-d₆): δ 11.61 (s, 1H), 7.91 (dd, 1H), 7.78-7.73 (m, 1H), 7.61-7.57 (m, 1H), 7.45-7.19 (m, 3H), 5.63 (s, 1H), 4.58 (d, 1H), 4.46 (d, 1H), 4.29 (d, 1H), 4.02 (dd, 1H), 2.66 (s, 3H); Chiral analytical SFC: RT=1.1 min, Column: Chiralpak IA-3 (4.6×150 mm) 3 μm, 30% EtOH, Flow rate: 3.0 g/min.

Enantiomer II (Compound 179): LCMS: m/z found 439.3 [M+H]⁺, RT=6.07 min, (Method A); ¹H NMR (400 MHz, DMSO-d₆): δ 11.61 (s, 1H), 7.91 (dd, 1H), 7.78-7.73 (m, 1H), 7.61-7.57 (m, 1H), 7.45-7.19 (m, 3H), 5.63 (s, 1H), 4.58 (d, 1H), 4.46 (d, 1H), 4.29 (d, 1H), 4.02 (dd, 1H), 2.66 (s, 3H); Chiral analytical SFC: RT=2.94 min, Column: Chiralpak IA-3 (4.6×150 mm) 3 μm, 30% EtOH, Flow rate: 3.0 g/min.

### 4-(Difluoromethyl)-N-(8-fluoro-6-oxo-1,4,5,6-tetrahydro-2H-pyrano[3,4-c]isoquinolin-1-yl)-N-methyl-1H-indole-2-carboxamide (Compounds 181 and 182)

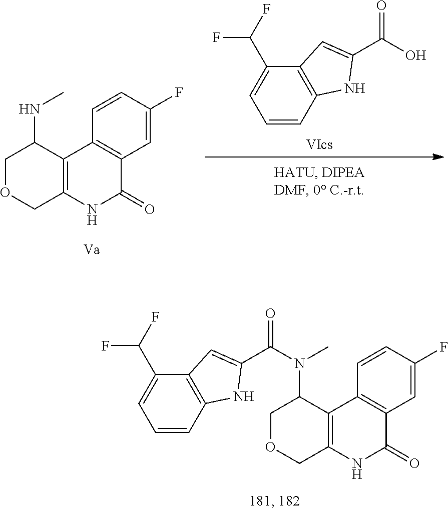

Racemic 4-(difluoromethyl)-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from racemic 8-fluoro-1-(methylamino)-1,5-dihydro-2H-pyrano[3,4-c]isoquinolin-6(4H)-one (Va) and 4-(difluoromethyl)-1H-indole-2-carboxylic acid (VIcs). The enantiomers were subsequently separated by preparative SFC: Method isocratic, Mobile phase MeOH:CO₂—40:60. Column: Chiralpak-AS-H (30×250 mm), 5 μm, flow rate: 100 g/min.

Enantiomer I (Compound 181): LCMS: m/z found 442.3 [M+H]⁺, RT=7.00 min, (Method A); ¹H NMR (400 MHz, DMSO-d₆): δ 12.05 (s, 1H), 11.64 (s, 1H), 7.92 (dd, 1H), 7.64 (d, 2H), 7.55 (br s, 1H), 7.42-7.11 (m, 3H), 7.00 (s, 1H), 5.76 (s, 1H), 4.63 (d, 1H), 4.49 (d, 1H), 4.02 (d, 1H), 3.14 (s, 3H); Chiral analytical SFC: RT=1.74 min, Column: Chiralpak AS-3 (4.6×150 mm) 3 μm, 40% MeOH, Flow rate: 3.0 g/min.

Enantiomer II (Compound 182): LCMS: m/z found 442.3 [M+H]⁺, RT=7.00 min, (Method A); ¹H NMR (400 MHz, DMSO-d₆): δ 12.05 (s, 1H), 11.64 (s, 1H), 7.92 (dd, 1H), 7.64 (d, 2H), 7.55 (br s, 1H), 7.42-7.11 (m, 3H), 7.00 (s, 1H), 5.76 (s, 1H), 4.63 (d, 1H), 4.49 (d, 1H), 4.02 (d, 1H), 3.14 (s, 3H); Chiral analytical SFC: RT=2.42 min, Column: Chiralpak AS-3 (4.6×150 mm) 3 μm, 40% MeOH, Flow rate: 3.0 g/min.

### 8,9-Difluoro-2H-pyrano[3,4-c]isoquinoline-1,6(4H,5H)-dione (IVb)

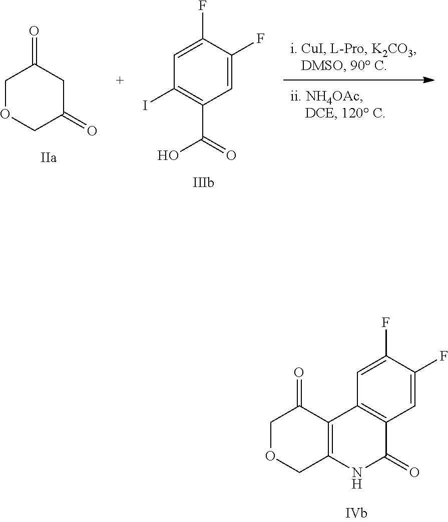

Step i: 4,5-Difluoro-2-iodo-benzoic acid (IIIb, 7.50 g, 26.4 mmol), tetrahydropyran-3,5-dione (IIa, 7.53 g, 66.0 mmol), copper (I) iodide (0.50 g, 2.64 mmol), L-Proline (0.61 g, 5.28 mmol), and potassium carbonate (21.3 g, 92.43 mmol) were combined in a 250 mL round-bottom flask, which was then evacuated and back-filled with nitrogen. Anhydrous DMSO (90 mL) was added and the reaction mixture was purged with nitrogen, and stirred under a nitrogen atmosphere at room temperature for 10 min, then at 90° C. (preheated bath temperature) for 4 h. The reaction mixture was cooled to room temperature, diluted slowly with water until homogeneous, and then acidified with 2 M aqueous HCl to pH<2 at 0° C., and extracted with ethyl acetate (3×400 mL). The combined organic extracts were washed with 5% brine 3 times and with saturated brine once, dried on sodium sulfate, and the solvent was evaporated under vacuum to a residue, which was further dried by azeotropic evaporation with toluene (50 mL), and then on high vacuum overnight, to provide crude 8,9-difluoro-4H-pyrano[3,4-c]isochromene-1,6-dione, which was used in the next step without further purification. ¹H NMR (400 MHz, DMSO-d₆) δ 8.72 (ddd, 1H), 8.25 (ddd, 1H), 4.82 (s, 2H), 4.35 (d, 2H).

Step ii: The crude 8,9-difluoro-4H-pyrano[3,4-c]isochromene-1,6-dione obtained in the step above and ammonium acetate (10.2 g, 132.1 mmol) were stirred in 1,2-dichloroethane (150 mL) at 120° C., in a sealed tube for 5 h. The volatiles were evaporated under vacuum, and the residue was suspended in water and stirred for 15 min, then the product was collected by filtration, washed with water, followed by methanol, and then by diethyl ether, and dried under high vacuum overnight to provide 8,9-Difluoro-2H-pyrano[3,4-c]isoquinoline-1,6(4H,5H)-dione (4.53 g, 68%). 252.2 [M+H]⁺; RT=0.74 min (Method B); ¹H NMR (400 MHz, DMSO-d₆) δ 12.33 (s, 1H), 8.90 (dd, 1H), 8.08 (dd, 1H), 4.77 (s, 2H), 4.27 (s, 2H).

### 8,9-Difluoro-1-(methylamino)-1,2,4,5-tetrahydropyrano[3,4-c]isoquinolin-6-one (Vb)

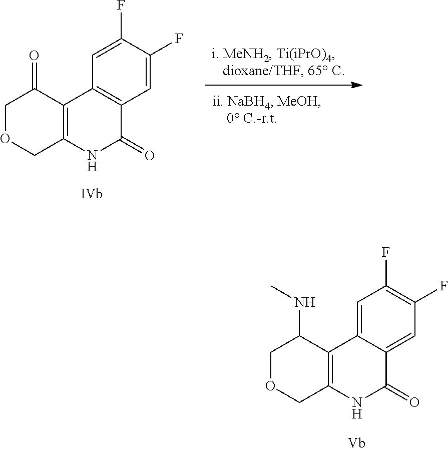

8,9-Difluoro-1-(methylamino)-1,2,4,5-tetrahydropyrano[3,4-c]isoquinolin-6-one was synthesized in an analogous manner as described above for Va, in 87% yield, from 8,9-difluoro-2H-pyrano[3,4-c]isoquinoline-1,6(4H,5H)-dione (IVb) and methylamine. LCMS m/z found 267.1 [M+H]⁺; RT=0.45 min (Method B); ¹H NMR (400 MHz, CDCl₃) δ 11.40 (s, 1H), 8.16 (dd, 1H), 7.54 (dd, 1H), 4.66 (d, 1H), 4.56 (d, 1H), 4.43 (d, 1H), 3.63 (dd, 1H), 3.49 (d, 1H), 2.61 (s, 3H).

### N-(8,9-Difluoro-6-oxo-1,4,5,6-tetrahydro-2H-pyrano[3,4-c]isoquinolin-1-yl)-5-fluoro-N-methylindoline-2-carboxamide (Compounds 5, 6, 7, and 8)

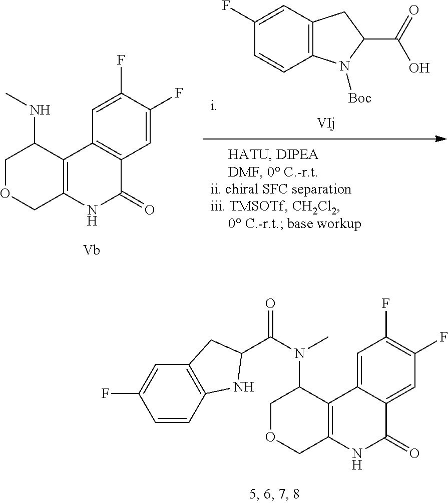

Step i: To a stirred solution of 530 mg (18.8 mmol, 1.0 eq.) of 1-(tert-butoxycarbonyl)-5-fluoroindoline-2-carboxylic acid (VIj) in 10 mL of DMF at room temperature, 1 mL (56.4 mmol, 3.0 eq.) of DIPEA, and 1.07 g (28.2 mmol, 1.5 eq.) of HATU were added and the mixture was stirred for 15 minutes. To this mixture, 500 mg (18.8 mmol, 1.0 eq.) of 8,9-difluoro-1-(methylamino)-1,5-dihydro-2H-pyrano[3,4-c]isoquinolin-6(4H)-one (Vb) was added and the resulting mixture was stirred at room temperature for 16 h. After completion of reaction (by TLC), the reaction mixture was diluted with water (30 mL) and filtered. The resulting solid product was washed with pentane (2×10 mL) to obtain tert-butyl 2-((8,9-difluoro-6-oxo-1,4,5,6-tetrahydro-2H-pyrano[3,4-c]isoquinolin-1-yl)(methyl)carbamoyl)-5-fluoroindoline-1-carboxylate as a mixture of four stereoisomers (710 mg).

Step ii: Chiral preparative SFC fractionation of this mixture: method isocratic, mobile phase methanol:CO₂—15:85. Column: Chiralpak AS (30×250 mm), 5 μm, flow rate: 100 g/min afforded two fractions: A (300 mg, mixture of 2 isomers) and B (300 mg, mixture of 2 isomers). Individual stereoisomers were subsequently separated by chiral preparative SFC fractionation of each of these two fractions (A and B): method isocratic, mobile phase methanol:CO₂—25:75. Column: Chiralpak IG (30×250 mm), 5 μm, flow rate: 100 g/min.

Step iii: Intermediate stereoisomers of tert-butyl 2-((8,9-difluoro-6-oxo-1,4,5,6-tetrahydro-2H-pyrano[3,4-c]isoquinolin-1-yl)(methyl)carbamoyl)-5-fluoroindoline-1-carboxylate isolated as described above were each converted to the final product in an analogous manner as describe above for Compounds 3 and 4 (Step ii). Diastereomeric pairs of separated enantiomers were assigned based on LCMS retention time and ¹H NMR identity.

Stereoisomer Ia (Compound 5): purified by trituration with 20 mL of diethyl ether. LCMS: m/z found 430.2 [M+H]⁺, RT=3.00 min, (Method A); ¹H NMR (400 MHz, DMSO-d₆): δ 11.65 (s, 1H), 8.13-8.08 (m, 1H), 7.08-7.03 (m, 1H), 6.85-6.83 (m, 1H), 6.78-6.73 (m, 1H), 6.58-6.55 (m, 1H), 5.66 (s, 1H), 5.50 (s, 1H), 4.75-4.71 (m, 1H), 4.61-4.57 (d, 1H), 4.44-4.40 (d, 1H), 4.03-4.00 (d, 1H), 3.96-3.93 (m, 1H), 3.29-3.26 (m, 1H), 2.91-2.85 (m, 4H); Chiral analytical SFC: RT=2.15 min, Column: Chiralpak AS-H3 (4.6×150 mm) 3 μm, 40% of Methanol (with 0.2% DEA as a modifier), Flow rate: 3.0 g/min.

Stereoisomer IIa (Compound 6, enantiomer of Compound 5): purified by trituration with diethyl ether (20 mL), followed by preparative HPLC (Column: X BRIDGE (19×250, 5 μm) Mobile phase A: 10 mM ammonium biarbonate in water, Mobile phase B: Acetonitrile, Gradient, Flow rate: 15 ml/min. LCMS: m/z found 430.2 [M+H]⁺, RT=3.00 min, (Method A); ¹H NMR (400 MHz, DMSO-d₆): δ 11.65 (s, 1H), 8.13-8.08 (m, 1H), 7.08-7.03 (m, 1H), 6.85-6.83 (m, 1H), 6.78-6.73 (m, 1H), 6.58-6.55 (m, 1H), 5.66 (s, 1H), 5.50 (s, 1H), 4.75-4.71 (m, 1H), 4.61-4.57 (d, 1H), 4.44-4.40 (d, 1H), 4.03-4.00 (d, 1H), 3.96-3.93 (m, 1H), 3.29-3.26 (m, 1H), 2.91-2.85 (m, 4H); Chiral analytical SFC: RT=4.04 min, Column: Chiralpak AS-H3 (4.6×150 mm) 3 μm, 40% of Methanol (with 0.2% DEA as a modifier), Flow rate: 3.0 g/min.

Stereoisomer Ib (Compound 7): purified by trituration with 10 mL of diethyl ether. LCMS: m/z found 430.2 [M+H]⁺, RT=2.83 min, (Method A); ¹H NMR (400 MHz, DMSO-d₆): δ 11.65 (s, 1H), 8.13-8.08 (m, 1H), 7.30-7.25 (m, 1H), 6.89-6.86 (m, 1H), 6.77-6.72 (m, 1H), 6.54-6.51 (m, 1H), 5.71 (s, 1H), 5.53 (s, 1H), 4.75-4.71 (m, 1H), 4.61-4.57 (d, 1H), 4.44-4.40 (d, 1H), 4.01-3.98 (d, 1H), 3.92-3.89 (m, 1H), 3.37-3.20 (m, 2H), 2.86 (s, 3H); Chiral analytical SFC: RT=3.07 min, Column: Chiralpak AS-H3 (4.6×150 mm) 3 μm, 25% of Methanol (with 0.2% DEA as a modifier), Flow rate: 3.0 g/min.

Stereoisomer Ib (Compound 8, enantiomer of Compound 7): purified by trituration with 10 mL of diethyl ether. LCMS: m/z found 430.2 [M+H]⁺, RT=2.83 min, (Method A); ¹H NMR (400 MHz, DMSO-d₆): δ 11.65 (s, 1H), 8.14-8.09 (m, 1H), 7.31-7.26 (m, 1H), 6.98-6.96 (m, 1H), 6.85-6.83 (m, 1H), 6.74-6.71 (m, 1H), 5.54 (s, 1H), 4.88-4.84 (m, 1H), 4.61-4.57 (d, 1H), 4.45-4.41 (d, 1H), 4.04-4.01 (d, 1H), 3.92-3.89 (m, 1H), 3.46-3.39 (m, 1H), 3.30-3.24 (m, 1H), 2.87 (s, 3H); Chiral analytical SFC: RT=3.65 min, Column: Chiralpak AS-H3 (4.6×150 mm) 3 μm, 40% of Methanol (with 0.2% DEA as a modifier), Flow rate: 3.0 g/min.

### N-(8,9-Difluoro-6-oxo-1,4,5,6-tetrahydro-2H-pyrano[3,4-c]isoquinolin-1-yl)-4,6-difluoro-N-methylindoline-2-carboxamide (Compounds 13, 14, 15, 16)

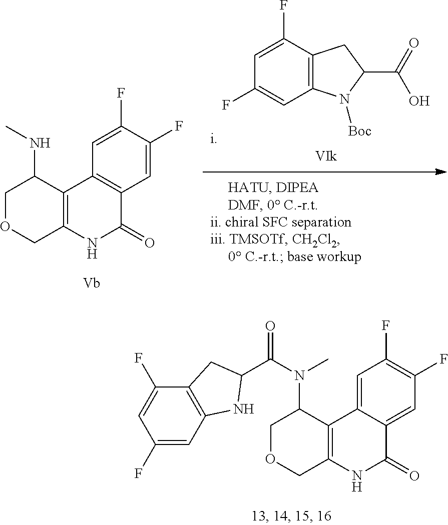

N-(8,9-Difluoro-6-oxo-1,4,5,6-tetrahydro-2H-pyrano[3,4-c]isoquinolin-1-yl)-4,6-difluoro-N-methylindoline-2-carboxamide was synthesized in an analogous manner as described above from racemic 8,9-difluoro-1-(methylamino)-1,5-dihydro-2H-pyrano[3,4-c]isoquinolin-6(4H)-one (Vb) and racemic 1-(tert-butoxycarbonyl)-4,6-difluoroindoline-2-carboxylic acid (VIk).

Stereoisomer Ia (Compound 13): LCMS: m/z found 448.2 [M+H]⁺, RT=3.94 min, (Method A); ¹H NMR (400 MHz, DMSO-d₆): δ 11.67 (s, 1H), 8.14-8.09 (m, 1H), 7.08-7.03 (m, 1H), 6.62 (m, 1H, exchangeable), 6.27-6.22 (m, 2H), 5.50 (m, 1H), 4.89-4.86 (m, 1H), 4.61-4.57 (m, 1H), 4.45-4.41 (m, 1H), 4.04-3.94 (m, 2H), 3.28-3.25 (m, 1H), 2.85-2.75 (m, 4H); Chiral analytical SFC: RT=1.70 min, Column: Chiralpak IC-3 (4.6×150 mm) 3 μm, 40% of Methanol (with 0.2% DEA as a modifier), Flow rate: 3.0 g/min.

Stereoisomer IIa (Compound 14, enantiomer of 13): LCMS: m/z found 448.2 [M+H]⁺, RT=3.94 min, (Method A); ¹H NMR (400 MHz, DMSO-d₆): δ 11.67 (s, 1H), 8.14-8.09 (m, 1H), 7.08-7.03 (m, 1H), 6.51 (m, 1H, exchangeable), 6.27-6.22 (m, 2H), 5.50 (m, 1H), 4.89-4.86 (m, 1H), 4.61-4.57 (m, 1H), 4.45-4.41 (m, 1H), 4.04-3.94 (m, 2H), 3.28-3.25 (m, 1H), 2.85-2.75 (m, 4H), 3.29-3.26 (m, 1H), 2.91-2.85 (m, 4H); Chiral analytical SFC: RT=2.15 min, Column: Chiralpak IC-3 (4.6×150 mm) 3 μm, 40% of Methanol (with 0.2% DEA as a modifier), Flow rate: 3.0 g/min.

Stereoisomer Ib (Compound 15): LCMS: m/z found 448.2 [M+H]⁺, RT=3.98 min, (Method A); ¹H NMR (400 MHz, DMSO-d₆): δ 11.67 (s, 1H), 8.14-8.09 (m, 1H), 7.28-7.23 (m, 1H), 6.51 (m, 1H, exchangeable), 6.25-6.17 (m, 2H), 5.53 (m, 1H), 4.89-4.85 (m, 1H), 4.61-4.57 (m, 1H), 4.45-4.41 (m, 1H), 4.05-4.02 (m, 1H), 3.92-3.89 (m, 1H), 3.34-3.30 (m, 1H), 3.19-3.15 (m, 1H), 2.77 (s, 3H); Chiral analytical SFC: RT=4.70 min, Column: Chiralpak IC-3 (4.6×150 mm) 3 μm, 25% of Methanol (with 0.2% DEA as a modifier), Flow rate: 3.0 g/min.

Stereoisomer IIb (Compound 15, enantiomer of 16): LCMS: m/z found 448.2 [M+H]⁺, RT=3.98 min, (Method A); ¹H NMR (400 MHz, DMSO-d₆): δ 11.67 (s, 1H), 8.14-8.09 (m, 1H), 7.28-7.23 (m, 1H), 6.53 (m, 1H, exchangeable), 6.25-6.17 (m, 2H), 5.53 (m, 1H), 4.89-4.85 (m, 1H), 4.61-4.57 (m, 1H), 4.45-4.41 (m, 1H), 4.05-4.02 (m, 1H), 3.92-3.89 (m, 1H), 3.34-3.30 (m, 1H), 3.19-3.15 (m, 1H), 2.86 (s, 3H); Chiral analytical SFC: RT=4.41 min, Column: Chiralpak IC-3 (4.6×150 mm) 3 μm, 40% of Methanol (with 0.2% DEA as a modifier), Flow rate: 3.0 g/min.

### N-(8,9-Difluoro-6-oxo-1,4,5,6-tetrahydro-2H-pyrano[3,4-c]isoquinolin-1-yl)-N,3,3-trimethylindoline-2-carboxamide (Compounds 56, 57, 58, 59)

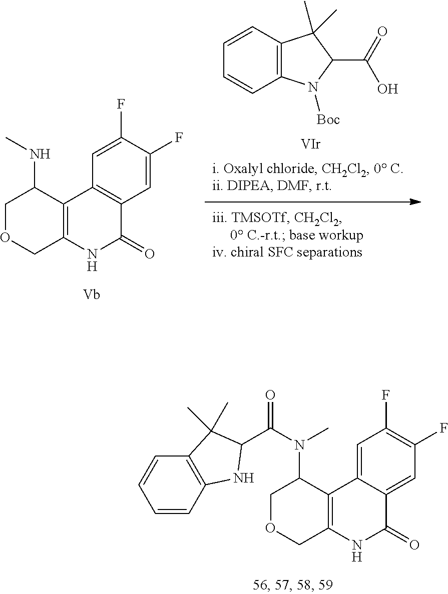

Step i: To a stirred solution of 100 mg (0.34 mmol, 1.0 eq.) of 1-(tert-butoxycarbonyl)-3,3-dimethylindoline-2-carboxylic acid (VIr, single enantiomer) in 2.0 mL of dichloromethane at 0° C., 65 μL (0.686 mmol, 2.0 eq.) of oxalyl chloride was added and the reaction was stirred at room temperature for 1 h. After completion of the reaction the reaction mixture was concentrated under reduced pressure and further azeotropped with toluene (2×5 mL) to obtain a brown syrup which was diluted with dry dichloromethane (2 mL) and added to a stirred solution of 90 mg (0.34 mmol, 1.0 eq.) of 8,9-difluoro-1-(methylamino)-1,5-dihydro-2H-pyrano[3,4-c]isoquinolin-6(4H)-one (Vb) in 1 mL of DMF at 0° C. and the mixture was stirred at room temperature for 2 h. The dichloromethane was removed under reduced pressure and the reaction mixture was poured in to ice-cold water (20 mL). The precipitated solid was filtered and washed with water (20 m L). The crude product was triturated with n-pentane (10 mL) to afford 180 mg (0.33 mmol, 60%) of tert-butyl 2-((8,9-difluoro-6-oxo-1,4,5,6-tetrahydro-2H-pyrano[3,4-c]isoquinolin-1-yl)(methyl)carbamoyl)-3,3-dimethylindoline-1-carboxylate (mixture of two diastereoisomers) as an off-white solid.

Step ii: tert-Butyl 2-((8,9-difluoro-6-oxo-1,4,5,6-tetrahydro-2H-pyrano[3,4-c]isoquinolin-1-yl)(methyl)carbamoyl)-3,3-dimethylindoline-1-carboxylate was converted to N-(8,9-difluoro-6-oxo-1,4,5,6-tetrahydro-2H-pyrano[3,4-c]isoquinolin-1-yl)-N,3,3-trimethyl-indoline-2-carboxamide in an analogous manner as describe above for Compounds 3 and 4 (Step ii). Diastereoisomers were subsequently separated by preparative SFC: method isocratic, mobile phase methanol:CO₂—40:60. Column: DCPAK P4CP (21×250 mm), 5 μm, flow rate: 70 g/min.

1-(tert-Butoxycarbonyl)-3,3-dimethylindoline-2-carboxylic acid (the opposite enantiomer of VIr) was converted to the remaining stereoisomers of N-(8,9-difluoro-6-oxo-1,4,5,6-tetrahydro-2H-pyrano[3,4-c]isoquinolin-1-yl)-N,3,3-trimethyl-indoline-2-carboxamide in an analogous manner as described above. Diastereomeric pairs of separated enantiomers were assigned based on LCMS retention time and ¹H NMR identity.

Diastereoisomer IIa (Compound 56): LCMS: m/z found 440.2 [M+H]⁺, RT=3.24 min, (Method A); ¹H NMR (400 MHz, DMSO-d₆): δ 11.5 (s, 1H), 8.11 (m, 1H), 7.29 (m, 1H), 6.93 (m, 2H), 6.56 (m, 2H), 5.77 (s, 1H), 5.52 (s, 1H), 4.57 (d, 1H), 4.46 (s, 1H), 4.45 (d, 1H), 3.95-3.87 (m, 2H), 2.88 (s, 3H), 1.36 (s, 3H), 1.26 (s, 3H); Chiral analytical SFC: RT=3.14 min, Column: Chiralcel OD-3 (4.6×150 mm) 3 μm, 30% of (0.2% 7 M Methanolic Ammonia in Acetonitrile:Methanol, 1:1), Flow rate: 3.0 g/min.

Diastereoisomer Ia (Compound 57, enantiomer of Compound 56): LCMS: m/z found 440.2 [M+H]⁺, RT=3.24 min, (Method A); ¹H NMR (400 MHz, DMSO-d₆ δ 11.5 (s, 1H), 8.11 (m, 1H), 7.29 (m, 1H), 6.93 (m, 2H), 6.56 (m, 2H), 5.77 (s, 1H), 5.52 (s, 1H), 4.57 (d, 1H), 4.46 (s, 1H), 4.45 (d, 1H), 3.95-3.87 (m, 2H), 2.88 (s, 3H), 1.36 (s, 3H), 1.26 (s, 3H); Chiral analytical SFC: RT=2.27 min, Column: Chiralcel OD-3 (4.6×150 mm) 3 μm, 30% of (0.2% 7 M Methanolic Ammonia in Acetonitrile:Methanol, 1:1), Flow rate: 3.0 g/min.

Diastereoisomer Ib (Compound 58): LCMS: m/z found 440.3 [M+H]⁺, RT=3.51 min, (Method A); ¹H NMR (400 MHz, DMSO-d₆): δ 11.64 (s, 1H), 8.05 (t, 1H), 7.21 (m, 1H), 6.93 (m, 2H), 6.55 (m, 2H), 5.68 (s, 1H), 5.51 (s, 1H), 4.53 (d, 1H), 4.46 (s, 1H), 4.40 (d, 1H), 4.05 (d, 1H), 3.90 (dd, 1H), 2.87 (s, 3H), 1.28 (s, 3H), 1.17 (s, 3H); Chiral analytical SFC: RT=2.09 min, Column: (R,R)-Whelk-01 (4.6×150 mm) 3 μm, 30% of (0.2% 7M Methanolic Ammonia in Acetonitrile:Methanol, 1:1), Flow rate: 3.0 g/min.

Diastereoisomer Ib (Compound 59, enantiomer of Compound 58): LCMS: m/z found 440.2 [M+H]⁺, RT=3.49 min, (Method A); ¹H NMR (400 MHz, DMSO-d₆): δ 11.64 (s, 1H), 8.05 (t, 1H), 7.21 (m, 1H), 6.93 (m, 2H), 6.55 (m, 2H), 5.68 (s, 1H), 5.51 (s, 1H), 4.53 (d, 1H), 4.46 (s, 1H), 4.40 (d, 1H), 4.05 (d, 1H), 3.90 (dd, 1H), 2.87 (s, 3H), 1.28 (s, 3H), 1.17 (s, 3H); Chiral analytical SFC: RT=2.56 min, Column: (R,R)-Whelk-01 (4.6×150 mm) 3 μm, 30% of (0.2% 7 M Methanolic Ammonia in Acetonitrile:Methanol, 1:1), Flow rate: 3.0 g/min.

### N-(8,9-Difluoro-6-oxo-1,4,5,6-tetrahydro-2H-pyrano[3,4-c]isoquinolin-1-yl)-8-fluoro-N-methylindolizine-2-carboxamide (Compounds 9 and 10)

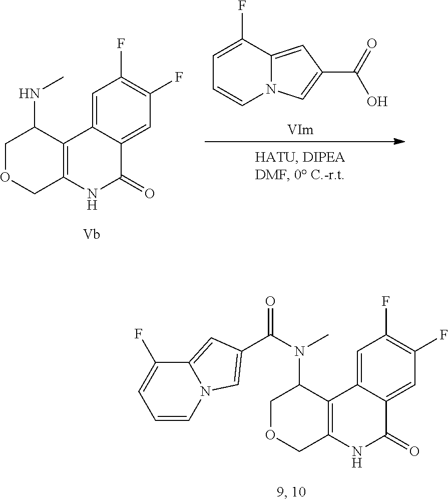

To a stirred solution of 135 mg (0.75 mmol, 1.0 eq.) of 8-fluoroindolizine-2-carboxylic acid (VIm) in 5 mL of DMF at room temperature were added 0.4 mL (2.25 mmol, 3.0 eq.) of DIPEA and 343 mg (0.90 mmol, 1.2 eq.) of HATU and the mixture was stirred for 15 minutes. To this mixture was added 200 mg (0.75 mmol, 1.0 eq.) of racemic 8,9-difluoro-1-(methylamino)-1,5-dihydro-2H-pyrano[3,4-c]isoquinolin-6(4H)-one (Vb) and the resulting mixture was stirred at room temperature for 16 h. The reaction mixture was then diluted with water (20 mL) and the precipitated solid was collected by filtration. After drying under vacuum, the resulting solid was triturated with pentane (15 mL) and filtered to obtain 258 mg (0.60 mmol, 78%) of racemic N-(8,9-difluoro-6-oxo-1,4,5,6-tetrahydro-2H-pyrano[3,4-c]isoquinolin-1-yl)-8-fluoro-N-methylindolizine-2-carboxamide. The enantiomers were subsequently separated by preparative SFC: Method isocratic, Mobile phase methanol:CO₂—40:60. Column Chiralpak IC (30×250 mm), 5 μm, flow rate: 105 g/min.

Enantiomer I (Compound 9): LCMS: m/z found 428.2 [M+H]⁺, RT=3.80 min, (Method A); ¹H NMR (400 MHz, DMSO-d₆): δ 11.69 (brs, 1H), 8.14-8.09 (m, 3H), 7.46-7.41 (m, 1H), 6.77 (s, 1H), 6.63 (d, 2H), 5.71 (s, 1H), 4.61 (d, 1H), 4.46 (d, 1H), 4.16 (d, 1H), 4.03 (d, 1H), 3.02 (s, 3H); Chiral analytical SFC: RT=3.89 min, Column: Chiralpak IC-3 (4.6×150 mm) 3 μm, 40% of Methanol, Flow rate: 3.0 g/min.

Enantiomer II (Compound 10): LCMS: m/z found 428.2 [M+H]⁺, RT=3.80 min, (Method A); ¹H NMR (400 MHz, DMSO-d₆): δ 11.69 (brs, 1H), 8.14-8.09 (m, 3H), 7.46-7.41 (m, 1H), 6.77 (s, 1H), 6.63 (d, 2H), 5.71 (s, 1H), 4.61 (d, 1H), 4.46 (d, 1H), 4.16 (d, 1H), 4.03 (d, 1H), 3.02 (s, 3H); Chiral analytical SFC: RT=5.36 min, Column: Chiralpak IC-3 (4.6×150 mm) 3 μm, 30% of Methanol, Flow rate: 3.0 g/min.

### N-(8,9-Difluoro-6-oxo-1,4,5,6-tetrahydro-2H-pyrano[3,4-c]isoquinolin-1-yl)-N-methylindolizine-2-carboxamide (Compounds 11 and 12)

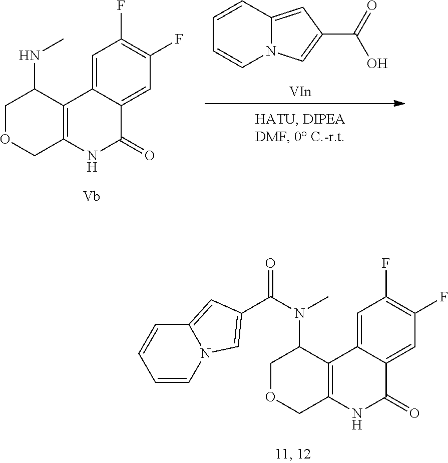

Racemic N-(8,9-difluoro-6-oxo-1,4,5,6-tetrahydro-2H-pyrano[3,4-c]isoquinolin-1-yl)-N-methylindolizine-2-carboxamide was synthesized in an analogous manner as described above from racemic 8,9-difluoro-1-(methylamino)-1,5-dihydro-2H-pyrano[3,4-c]isoquinolin-6(4H)-one (Vb) and indolizine-2-carboxylic acid (VIn). The enantiomers were subsequently separated by preparative SFC: Method isocratic, Mobile phase methanol:CO₂—40:60. Column: Chiralpak-IA (30×250 mm), 5 μm, flow rate: 110 g/min.

Enantiomer I (Compound 11): LCMS: m/z found 410.2 [M+H]⁺, RT=3.59 min, (Method A); ¹H NMR (400 MHz, DMSO-d₆): δ 11.69 (s, 1H), 8.24 (d, 1H), 8.14-8.09 (m, 1H), 7.92 (s, 1H), 7.47-7.41 (m, 2H), 6.75-6.72 (m, 1H), 6.64-6.59 (m, 2H), 5.72 (s, 1H), 4.63-4.59 (m, 1H), 4.48-4.44 (m, 1H), 4.17-4.14 (m, 1H), 4.04-4.02 (m, 1H), 3.03 (s, 3H); Chiral analytical SFC: RT=2.95 min, Column: Chiralpak-IA-3 (4.6×150 mm) 3 μm, 40% of Methanol, Flow rate: 3.0 g/min.

Enantiomer II (Compound 12): LCMS: m/z found 410.3 [M+H]⁺, RT=3.59 min, (Method A); ¹H NMR (400 MHz, DMSO-d₆): δ 11.69 (s, 1H), 8.24 (d, 1H), 8.14-8.09 (m, 1H), 7.92 (s, 1H), 7.47-7.41 (m, 2H), 6.75-6.72 (m, 1H), 6.64-6.59 (m, 2H), 5.72 (s, 1H), 4.63-4.59 (m, 1H), 4.48-4.44 (m, 1H), 4.17-4.14 (m, 1H), 4.04-4.02 (m, 1H), 3.03 (s, 3H); Chiral analytical SFC: RT=4.46 min, Column: Chiralpak-IA-3 (4.6×150 mm) 3 μm, 40% of Methanol, Flow rate: 3.0 g/min.

### 8-Chloro-N-(8,9-difluoro-6-oxo-1,4,5,6-tetrahydro-2H-pyrano[3,4-c]isoquinolin-1-yl)-N-methylindolizine-2-carboxamide (Compounds 17 and 18)

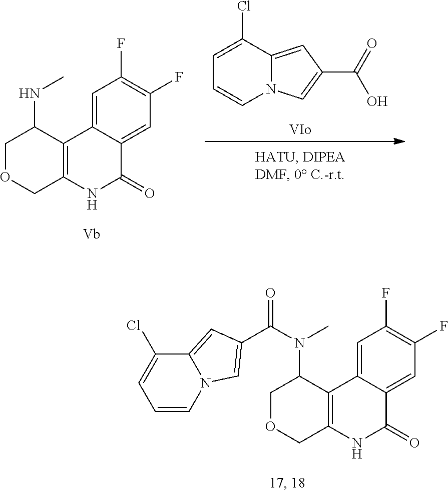

Racemic 8-chloro-N-(8,9-difluoro-6-oxo-1,4,5,6-tetrahydro-2H-pyrano[3,4-c]isoquinolin-1-yl)-N-methylindolizine-2-carboxamide was synthesized in an analogous manner as described above from racemic 8,9-difluoro-1-(methylamino)-1,5-dihydro-2H-pyrano[3,4-c]isoquinolin-6(4H)-one (Vb) and 8-chloroindolizine-2-carboxylic acid (VIo). The enantiomers were subsequently separated by preparative SFC: Method isocratic, Mobile phase methanol:CO₂—45:55. Column: Chiralpak IC (30×250 mm), 5μ, flow rate: 110 g/min.

Enantiomer I (Compound 17): LCMS: m/z found 444.2/446.2 [M+H]⁺, RT=4.11 min, (Method A); ¹H NMR (400 MHz, DMSO-d₆): δ 11.68 (br s, 1H), 8.27 (d, 1H), 8.12-8.07 (m, 2H), 7.44-7.39 (m, 1H), 6.95 (d, 1H), 6.73 (s, 1H), 6.66-6.62 (t, 1H), 5.71 (br s, 1H), 4.57 (d, 1H), 4.43 (d, 1H), 4.15 (d, 1H), 4.01 (d, 1H), 3.02 (s, 3H); Chiral analytical SFC: RT=3.54 min, Column: Chiralpak IC-3 (4.6×150 mm) 3 μm, 50% of Methanol, Flow rate: 3.0 g/min.

Enantiomer II (Compound 18): LCMS: m/z found 444.2/446.2 [M+H]⁺, RT=4.11 min, (Method A); ¹H NMR (400 MHz, DMSO-d₆): δ 11.68 (br s, 1H), 8.27 (d, 1H), 8.12-8.07 (m, 2H), 7.44-7.39 (m, 1H), 6.95 (d, 1H), 6.73 (s, 1H), 6.66-6.62 (t, 1H), 5.71 (br s, 1H), 4.57 (d, 1H), 4.43 (d, 1H), 4.15 (d, 1H), 4.01 (d, 1H), 3.02 (s, 3H); Chiral analytical SFC: RT=4.87 min, Column: Chiralpak IC-3 (4.6×150 mm) 3 μm, 50% of Methanol, Flow rate: 3.0 g/min.

### N-(8,9-Difluoro-6-oxo-1,4,5,6-tetrahydro-2H-pyrano[3,4-c]isoquinolin-1-yl)-N-methyl-1H-indole-2-carboxamide: (Compounds 21 and 22)

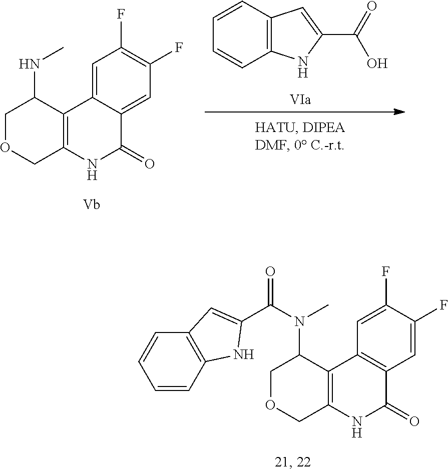

Racemic N-(8,9-difluoro-6-oxo-1,4,5,6-tetrahydro-2H-pyrano[3,4-c]isoquinolin-1-yl)-N-methyl-1H-indole-2-carboxamide was synthesized in an analogous manner as described above from racemic 8,9-difluoro-1-(methylamino)-1,5-dihydro-2H-pyrano[3,4-c]isoquinolin-6(4H)-one (Vb) and 1H-indole-2-carboxylic acid (VIa). The enantiomers were subsequently separated by preparative SFC: Method isocratic, Mobile phase methanol:CO₂—45:55. Column: Chiralpak IC (30×250 mm), 5 μm, flow rate: 100 g/min.

Enantiomer I (Compound 21): LCMS: m/z found 410.2 [M+H]⁺, RT=3.99 min, (Method A); ¹H NMR (400 MHz, DMSO-d₆): δ 11.71 (br s, 2H), 8.13-8.08 (m, 1H), 7.61 (d, 1H), 7.49-7.46 (m, 2H), 7.22 (t, 1H), 7.05 (t, 1H), 6.95-(s, 1H), 5.77 (d, 1H), 4.63 (d, 1H), 4.48 (d, 1H), 4.18-4.03 (m, 2H) 3.17 (s, 3H); Chiral analytical SFC: RT=3.57 min, Column: Chiralpak IC-3 (4.6×150 mm) 3 μm, 40% of Methanol, Flow rate: 3.0 g/min.

Enantiomer II (Compound 22): LCMS: m/z found 410.2 [M+H]⁺, RT=3.99 min, (Method A); ¹H NMR (400 MHz, DMSO-d₆): δ 11.71 (br s, 2H), 8.13-8.08 (m, 1H), 7.61 (d, 1H), 7.49-7.46 (m, 2H), 7.22 (t, 1H), 7.05 (t, 1H), 6.95 (s, 1H), 5.77 (d, 1H), 4.63 (d, 1H), 4.48 (d, 1H), 4.18-4.03 (m, 2H), 3.17 (s, 3H); Chiral analytical SFC: RT=6.17 min, Column: Chiralpak IC-3 (4.6×150 mm) 3 μm, 40% of Methanol, Flow rate: 3.0 g/min.

### N-(8,9-Difluoro-6-oxo-1,4,5,6-tetrahydro-2H-pyrano[3,4-c]isoquinolin-1-yl)-4-fluoro-N-methyl-1H-indole-2-carboxamide (Compounds 23 and 24)

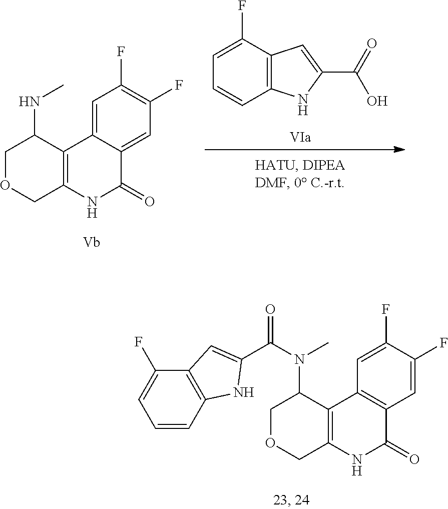

Racemic N-(8,9-difluoro-6-oxo-1,4,5,6-tetrahydro-2H-pyrano[3,4-c]isoquinolin-1-yl)-4-fluoro-N-methyl-1H-indole-2-carboxamide was synthesized in an analogous manner as described above from racemic 8,9-difluoro-1-(methylamino)-1,5-dihydro-2H-pyrano[3,4-c]isoquinolin-6(4H)-one (Vb) and 4-fluoro-1H-indole-2-carboxylic acid (VIc). The enantiomers were subsequently separated by preparative SFC: Method isocratic, Mobile phase methanol:CO₂—40:60. Column: Chiralpak IC (30×250 mm), 5 μm, flow rate: 110 g/min.

Enantiomer I (Compound 23): LCMS: m/z found 428.2 [M+H]⁺, RT=4.16 min, (Method A); ¹H NMR (400 MHz, DMSO-d₆): δ 12.1 (br s, 2H), 8.11-8.06 (m, 1H), 7.41 (m, 1H), 7.36 (d, 1H), 7.21-7.16 (m, 1H) 6.97 (s, 1H), 6.84-6.80 (m, 1H) 5.74 (br s, 1H), 4.58 (d, 1H), 4.46 (d, 1H), 4.15 (d, 1H), 4.02 (d, 1H), 3.16 (s, 3H); Chiral analytical SFC: RT=2.34 min, Column: Chiralpak IC-3 (4.6×150 mm) 3 μm, 40% of Methanol, Flow rate: 3.0 g/min.

Enantiomer II (Compound 24): LCMS: m/z found 428.2 [M+H]⁺, RT=4.16 min, (Method A); ¹H NMR (400 MHz, DMSO-d₆): δ 12.1 (br s, 2H), 8.11-8.06 (m, 1H), 7.41 (m, 1H), 7.36 (d, 1H), 7.21-7.16 (m, 1H) 6.97 (s, 1H), 6.84-6.80 (m, 1H) 5.74 (br s, 1H), 4.58 (d, 1H), 4.46 (d, 1H), 4.15 (d, 1H), 4.02 (d, 1H), 3.16 (s, 3H); Chiral analytical SFC: RT=3.03 min, Column: Chiralpak IC-3 (4.6×150 mm) 3 μm, 40% of Methanol, Flow rate: 3.0 g/min.

### N-(8,9-Difluoro-6-oxo-1,4,5,6-tetrahydro-2H-pyrano[3,4-c]isoquinolin-1-yl)-6-fluoro-N-methyl-1H-indole-2-carboxamide (Compounds 25 and 26)

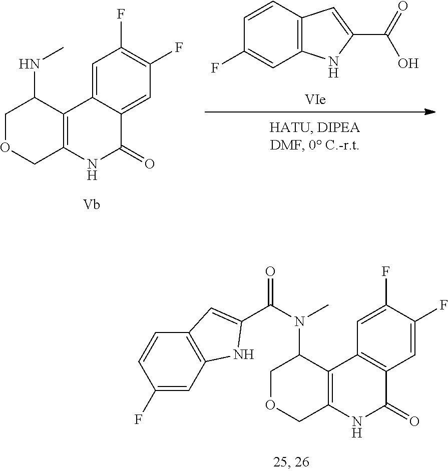

Racemic N-(8,9-difluoro-6-oxo-1,4,5,6-tetrahydro-2H-pyrano[3,4-c]isoquinolin-1-yl)-6-fluoro-N-methyl-1H-indole-2-carboxamide was synthesized in an analogous manner as described above from racemic 8,9-difluoro-1-(methylamino)-1,5-dihydro-2H-pyrano[3,4-c]isoquinolin-6(4H)-one (Vb) and 6-fluoro-1H-indole-2-carboxylic acid (VIe). The enantiomers were subsequently separated by preparative SFC: Method isocratic, Mobile phase methanol:CO₂—45:55. Column: Chiralpak IC (30×250 mm), 5 μm, flow rate: 110 g/min.

Enantiomer I (Compound 25): LCMS: m/z found 428.2 [M+H]⁺, RT=4.14 min, (Method A); ¹H NMR (400 MHz, DMSO-d₆) δ 12.1 (br s, 2H), 8.13-8.08 (m, 1H), 7.65-7.61 (m, 1H), 7.45-7.40 (m, 1H), 7.17 (d, 1H), 6.99 (s, 1H), 6.95-690 (m, 1H), 5.75 (br s, 1H), 4.61 (d, 1H), 4.45 (d, 1H), 4.14 (d, 1H), 4.02 (d, 1H), 3.16 (s, 3H); Chiral analytical SFC: RT=3.03 min, Column: Chiralpak IC-3 (4.6×150 mm) 3 μm, 40% of Methanol, Flow rate: 3.0 g/min.

Enantiomer II (Compound 26): LCMS: m/z found 428.2 [M+H]⁺, RT=4.14 min, (Method A); ¹H NMR (400 MHz, DMSO-d₆) δ 12.1 (br s, 2H), 8.13-8.08 (m, 1H), 7.65-7.61 (m, 1H), 7.45-7.40 (m, 1H), 7.17 (d, 1H), 6.99 (s, 1H), 6.95-690 (m, 1H), 5.75 (br s, 1H), 4.61 (d, 1H), 4.45 (d, 1H), 4.14 (d, 1H), 4.02 (d, 1H), 3.16 (s, 3H); Chiral analytical SFC: RT=3.73 min, Column: Chiralpak IC-3 (4.6×150 mm) 3 μm, 40% of Methanol, Flow rate: 3.0 g/min.

### N-(8,9-Difluoro-6-oxo-1,4,5,6-tetrahydro-2H-pyrano[3,4-c]isoquinolin-1-yl)-5-fluoro-N-methyl-1H-indole-2-carboxamide (Compounds 27 and 28)

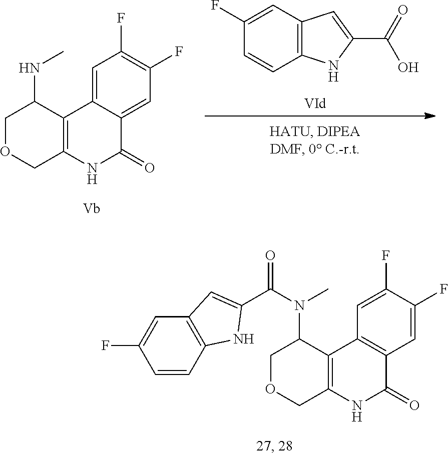

Racemic N-(8,9-difluoro-6-oxo-1,4,5,6-tetrahydro-2H-pyrano[3,4-c]isoquinolin-1-yl)-5-fluoro-N-methyl-1H-indole-2-carboxamide was synthesized in an analogous manner as described above from racemic 8,9-difluoro-1-(methylamino)-1,5-dihydro-2H-pyrano[3,4-c]isoquinolin-6(4H)-one (Vb) and 5-fluoro-1H-indole-2-carboxylic acid (VId). The enantiomers were subsequently separated by preparative SFC: Method isocratic, Mobile phase methanol:CO₂—40:60. Column: Chiralpak IC (30×250 mm), 5 μm, flow rate: 110 g/min.

Enantiomer I (Compound 27): LCMS: m/z found 428.2 [M+H]⁺, RT=4.10 min, (Method A); ¹H NMR (400 MHz, DMSO-d₆) δ 11.82 (br s, 1H), 8.12-8.07 (m, 1H), 7.48-7.35 (m, 3H), 7.10-7.05 (m, 1H), 6.92 (s, 1H), 5.74 (s, 1H), 4.63 (d, 1H), 4.46 (d, 1H), 4.16 (d, 1H), 4.03 (d, 1H), 3.15 (s, 3H); Chiral analytical SFC: RT=2.56 min, Column: Chiralpak IC-3 (4.6×150 mm) 3 μm, 40% of Methanol, Flow rate: 3.0 g/min.

Enantiomer II (Compound 28): LCMS: m/z found 428.2 [M+H]⁺, RT=4.10 min, (Method A); ¹H NMR (400 MHz, DMSO-d₆) δ 11.82 (br s, 1H), 8.12-8.07 (m, 1H), 7.48-7.35 (m, 3H), 7.10-7.05 (m, 1H), 6.92 (s, 1H), 5.74 (s, 1H), 4.63 (d, 1H), 4.46 (d, 1H), 4.16 (d, 1H), 4.03 (d, 1H), 3.15 (s, 3H); Chiral analytical SFC: RT=3.54 min, Column: Chiralpak IC-3 (4.6×150 mm) 3 μm, 40% of Methanol, Flow rate: 3.0 g/min.

### N-(8,9-Difluoro-6-oxo-1,4,5,6-tetrahydro-2H-pyrano[3,4-c]isoquinolin-1-yl)-7-fluoro-N-methyl-1H-indole-2-carboxamide (Compounds 39 and 40)

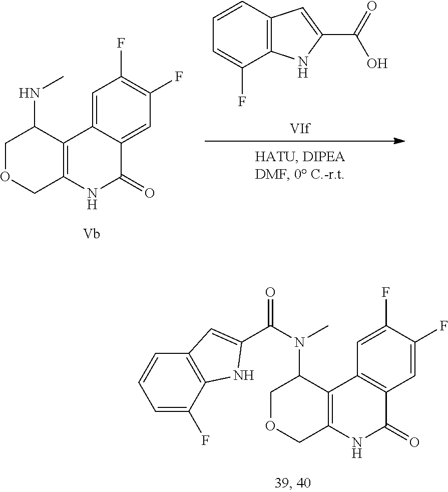

Racemic N-(8,9-difluoro-6-oxo-1,4,5,6-tetrahydro-2H-pyrano[3,4-c]isoquinolin-1-yl)-7-fluoro-N-methyl-1H-indole-2-carboxamide was synthesized in an analogous manner as described above from racemic 8,9-difluoro-1-(methylamino)-1,5-dihydro-2H-pyrano[3,4-c]isoquinolin-6(4H)-one (Vb) and 5-fluoro-1H-indole-2-carboxylic acid (VIf). The enantiomers were subsequently separated by preparative SFC: Method isocratic, Mobile phase methanol:CO₂—45:55. Column: Chiralpak IC (30×250 mm), 5 μm, flow rate: 110 g/min.

Enantiomer I (Compound 39): LCMS: m/z found 428.2 [M+H]⁺, RT=4.11 min, (Method A); ¹H NMR (400 MHz, DMSO-d₆) δ 12.12 (br s, 2H), 8.11 (t, 1H), 7.43-7.39 (m, 2H), 7.03-6.95 (m, 3H), 5.72 (s, 1H), 4.62 (d, 1H), 4.47 (d, 1H), 4.18 (d, 1H), 4.05 (d, 1H), 3.09 (s, 3H); Chiral analytical SFC: RT=2.95 min, Column: Chiralpak IC-3 (4.6×150 mm) 3 μm, 40% of Methanol, Flow rate: 3.0 g/min.

Enantiomer II (Compound 40): LCMS: m/z found 428.2 [M+H]⁺, RT=4.11 min, (Method A); ¹H NMR (400 MHz, DMSO-d₆) δ 12.03 (br s, 2H), 8.10 (t, 1H), 7.43-7.37 (m, 2H), 7.03-6.95 (m, 3H), 5.72 (s, 1H), 4.62 (d, 1H), 4.47 (d, 1H), 4.18 (d, 1H), 4.05 (d, 1H), 3.09 (s, 3H); Chiral analytical SFC: RT=5.04 min, Column: Chiralpak IC-3 (4.6×150 mm) 3 μm, 40% of Methanol, Flow rate: 3.0 g/min.

### N-(8,9-difluoro-6-oxo-1,4,5,6-tetrahydro-2H-pyrano[3,4-c]isoquinolin-1-yl)-5,6-difluoro-N-methyl-1H-indole-2-carboxamide (Compounds 41 and 42)

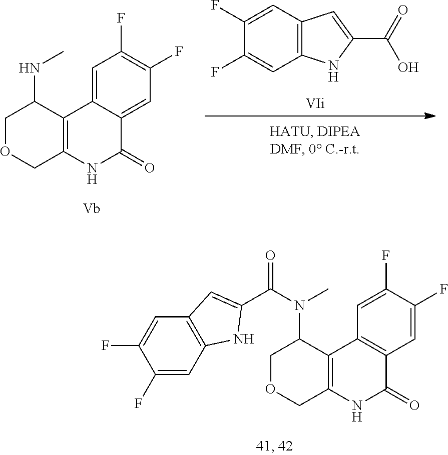

Racemic N-(8,9-difluoro-6-oxo-1,4,5,6-tetrahydro-2H-pyrano[3,4-c]isoquinolin-1-yl)-5,6-difluoro-N-methyl-1H-indole-2-carboxamide was synthesized in an analogous manner as described above (Compounds 9 and 10) from racemic 8,9-difluoro-1-(methylamino)-1,5-dihydro-2H-pyrano[3,4-c]isoquinolin-6(4H)-one (Vb) and 5,6-difluoro-1H-indole-2-carboxylic acid (VIi). The enantiomers were subsequently separated by preparative SFC: Method isocratic, Mobile phase methanol:CO₂—40:60. Column: Chiralpak IC (30×250 mm), 5 μm, flow rate: 110 g/min.

Enantiomer I (Compound 41): LCMS: m/z found 446.2 [M+H]⁺, RT=5.08 min, (Method A); ¹H NMR (400 MHz, DMSO-d₆) δ 11.92 (br s, 1H, exch.), 11.74 (br s, 1H, exch.), 8.12 (dd, 1H), 7.61 (dd, 1H), 7.41 (m, 2H), 6.97 (s, 1H), 5.75 (s, 1H), 4.64 (d, 1H), 4.53-4.43 (m, 1H), 4.17 (d, 1H), 4.08-3.99 (m, 1H), 3.15 (s, 3H); Chiral analytical SFC: RT=2.20 min, Column: Chiralpak IC-3 (4.6×150 mm) 3 μm, 40% of Methanol, Flow rate: 3.0 g/min.

Enantiomer II (Compound 42): LCMS: m/z found 446.2 [M+H]⁺, RT=5.08 min, (Method A); ¹H NMR (400 MHz, DMSO-d₆) δ 11.92 (br s, 1H, exch.), 11.74 (br s, 1H, exch.), 8.12 (dd, 1H), 7.61 (dd, 1H), 7.41 (m, 2H), 6.97 (s, 1H), 5.75 (s, 1H), 4.64 (d, 1H), 4.53-4.43 (m, 1H), 4.17 (d, 1H), 4.08-3.99 (m, 1H), 3.15 (s, 3H); Chiral analytical SFC: RT=2.69 min, Column: Chiralpak IC-3 (4.6×150 mm) 3 μm, 40% of Methanol, Flow rate: 3.0 g/min. Enantiomer II (Compound 42) was also independently prepared by chiral synthesis starting from enantiomerically pure (S)-8,9-difluoro-1-(methylamino)-1,5-dihydro-2H-pyrano[3,4-c]isoquinolin-6(4H)-one (Vb) (see elsewhere herein).

### N-(8,9-Difluoro-6-oxo-1,4,5,6-tetrahydro-2H-pyrano[3,4-c]isoquinolin-1-yl)-4,6-difluoro-N-methyl-1H-indole-2-carboxamide (Compounds 43 and 44)

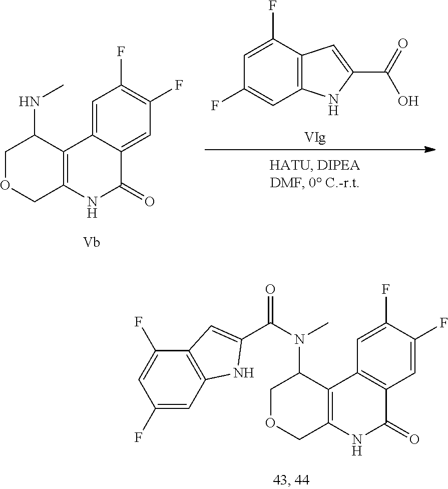

Racemic N-(8,9-difluoro-6-oxo-1,4,5,6-tetrahydro-2H-pyrano[3,4-c]isoquinolin-1-yl)-4,6-difluoro-N-methyl-1H-indole-2-carboxamide was synthesized in an analogous manner as described above from racemic 8,9-difluoro-1-(methylamino)-1,5-dihydro-2H-pyrano[3,4-c]isoquinolin-6(4H)-one (Vb) and 4,6-difluoro-1H-indole-2-carboxylic acid (VIg). The enantiomers were subsequently separated by preparative SFC: Method isocratic, Mobile phase methanol:CO₂—35:65. Column: Chiralpak IC (30×250 mm) 5 μm, flow rate: 110 g/min.

Enantiomer I (Compound 43): LCMS: m/z found 446.2 [M+H]⁺, RT=4.40 min, (Method A); ¹H NMR (400 MHz, DMSO-d₆) δ 11.77-12.11 (br s, 2H), 8.14-8.09 (m, 1H), 7.45-7.40 (m, 1H), 7.08-7.02 (m, 2H), 6.92-6.87 (m, 1H), 5.74 (s, 1H), 4.64 (d, 1H), 4.48 (d, 1H), 4.15 (d, 1H), 4.05 (d, 1H), 3.16 (s, 3H); Chiral analytical SFC: RT=1.87 min, Column: Chiralpak IC-3 (4.6×150 mm) 3 μm, 40% of Methanol, Flow rate: 3.0 g/min.

Enantiomer II (Compound 44): LCMS: m/z found 446.2 [M+H]⁺, RT=4.40 min, (Method A); ¹H NMR (400 MHz, DMSO-d₆) δ 11.95 (br s, 2H), 8.13-8.08 (m, 1H), 7.44-7.39 (m, 1H), 7.09-7.02 (m, 2H), 6.92-6.86 (m, 1H), 5.74 (s, 1H), 4.63 (d, 1H), 4.47 (d, 1H), 4.17 (d, 1H), 4.03 (d, 1H), 3.16 (s, 3H); Chiral analytical SFC: RT=2.21 min, Column: Chiralpak IC-3 (4.6×150 mm) 3 μm, 40% of Methanol, Flow rate: 3.0 g/min.

### N-(8,9-Difluoro-6-oxo-1,4,5,6-tetrahydro-2H-pyrano[3,4-c]isoquinolin-1-yl)-4,5-difluoro-N-methyl-1H-indole-2-carboxamide (Compounds 45 and 46)

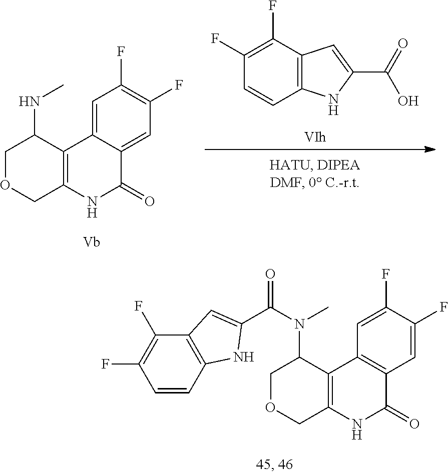

Racemic N-(8,9-difluoro-6-oxo-1,4,5,6-tetrahydro-2H-pyrano[3,4-c]isoquinolin-1-yl)-4,5-difluoro-N-methyl-1H-indole-2-carboxamide was synthesized in an analogous manner as described above from racemic 8,9-difluoro-1-(methylamino)-1,5-dihydro-2H-pyrano[3,4-c]isoquinolin-6(4H)-one (Vb) and 4,5-difluoro-1H-indole-2-carboxylic acid (VIh). The enantiomers were subsequently separated by preparative SFC: Method isocratic, Mobile phase methanol:CO₂—35:65. Column: Chiralpak IC (30×250 mm), 5 μm, flow rate: 110 g/min.

Enantiomer I (Compound 45): LCMS: m/z found 446.2 [M+H]⁺, RT=4.32 min, (Method A); ¹H NMR (400 MHz, DMSO-d₆) δ 12.1 (br s, 2H), 8.11-8.06 (m, 1H), 7.40-7.35 (m, 1H), 7.27-7.21 (m, 2H), 7.04 (s, 1H), 5.73 (br s, 1H), 4.58 (d, 1H), 4.43 (d, 1H), 4.18 (d, 1H), 4.02 (d, 1H), 3.16 (s, 3H); Chiral analytical SFC: RT=2.18 min, Column: Chiralpak IC-3 (4.6×150 mm) 3 μm, 40% of Methanol, Flow rate: 3.0 g/min.

Enantiomer II (Compound 46): LCMS: m/z found 446.2 [M+H]⁺, RT=4.32 min, (Method A); ¹H NMR (400 MHz, DMSO-d₆) δ 12.1 (br s, 2H), 8.11-8.06 (m, 1H), 7.40-7.35 (m, 1H), 7.27-7.21 (m, 2H), 7.04 (s, 1H), 5.73 (br s, 1H), 4.58 (d, 1H), 4.43 (d, 1H), 4.18 (d, 1H), 4.02 (d, 1H), 3.16 (s, 3H); Chiral analytical SFC: RT=2.54 min, Column: Chiralpak IC-3 (4.6×150 mm) 3 μm, 40% of Methanol, Flow rate: 3.0 g/min.

### N-(8,9-Difluoro-6-oxo-1,4,5,6-tetrahydro-2H-pyrano[3,4-c]isoquinolin-1-yl)-5,5-difluoro-N-methyl-4,5,6,7-tetrahydro-1H-indole-2-carboxamide (Compounds 47 and 48)

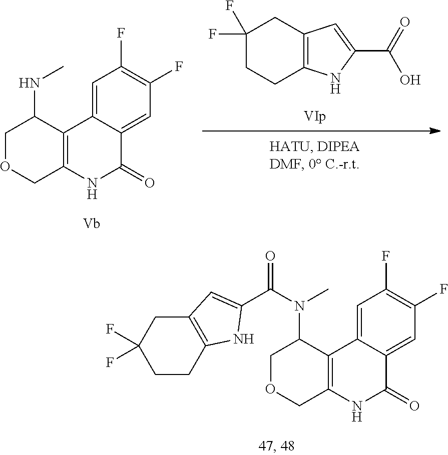

Racemic N-(8,9-difluoro-6-oxo-1,4,5,6-tetrahydro-2H-pyrano[3,4-c]isoquinolin-1-yl)-5,5-difluoro-N-methyl-4,5,6,7-tetrahydro-1H-indole-2-carboxamide was synthesized in an analogous manner as described above from racemic 8,9-difluoro-1-(methylamino)-1,5-dihydro-2H-pyrano[3,4-c]isoquinolin-6(4H)-one (Vb) and 5,5-difluoro-4,5,6,7-tetrahydro-1H-indole-2-carboxylic acid (VIp). The enantiomers were subsequently separated by preparative SFC: Method isocratic, Mobile phase acetonitrile/methanol (1:1, v/v):CO₂—30:60. Column: Chiralcel-OX-H (30×250 mm) 5 μm, flow rate: 100 g/min.

Enantiomer I (Compound 47): LCMS: m/z found 446.2 [M+H]⁺, RT=4.32 min, (Method A); ¹H NMR (400 MHz, DMSO-d₆) δ 11.65 (br s, 1H), 11.39 (s, 1H), 8.07 (t, 1H), 7.35 (br s, 1H), 6.39 (s, 1H), 5.68 (s, 1H), 4.58 (d, 1H), 4.42 (d, 1H), 4.05-3.97 (m, 2H), 3.02-2.98 (m, 5H), 2.76 (t, 2H), 2.23-2.18 (m, 2H); Chiral analytical SFC: RT=3.15 min, Column: Chiralcel-OX-3 (4.6×150 mm) 3 μm, 30% of (0.2% 7 M Methanolic Ammonia in Acetonitrile:Methanol 1:1), Flow rate: 3.0 g/min.

Enantiomer II (Compound 48): LCMS: m/z found 446.2 [M+H]⁺, RT=4.32 min, (Method A); ¹H NMR (400 MHz, DMSO-d₆ δ 11.65 (br s, 1H), 11.39 (s, 1H), 8.07 (t, 1H), 7.35 (br s, 1H), 6.39 (s, 1H), 5.68 (s, 1H), 4.58 (d, 1H), 4.42 (d, 1H), 4.05-3.97 (m, 2H), 3.02-2.98 (m, 5H), 2.76 (t, 2H), 2.23-2.18 (m, 2H); Chiral analytical SFC: RT=3.67 min, Column: Chiralcel-OX-3 (4.6×150 mm) 3 μm, 30% of (0.2% 7 M Methanolic Ammonia in Acetonitrile:Methanol 1:1), Flow rate: 3.0 g/min.

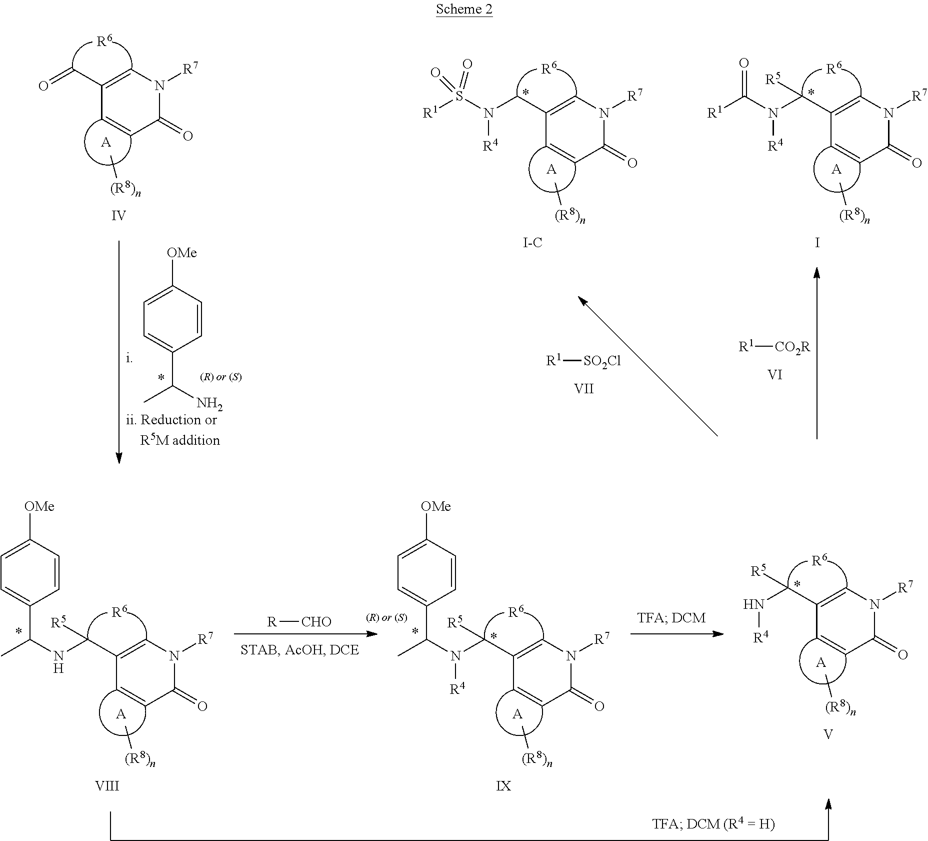

### (S)-8-Fluoro-1-(((R)-1-(4-methoxyphenyl)ethyl)amino)-1,5-dihydro-2H-pyrano[3,4-c]isoquinolin-6(4H)-one (VIIIa)

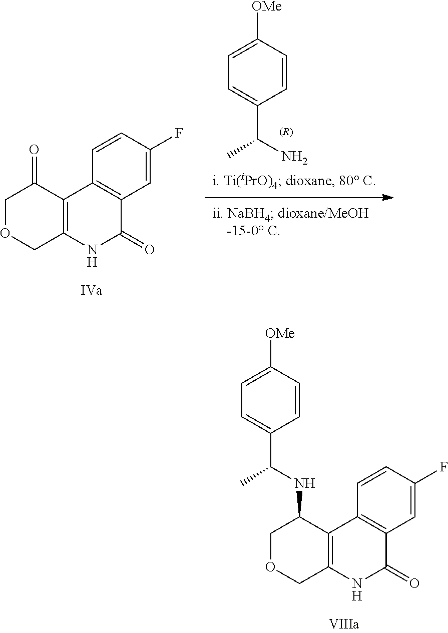

Tetraisopropoxytitanium (1.95 mL, 6.43 mmol) was added to a mixture of 8-fluoro-4,5-dihydropyrano[3,4-c]isoquinoline-1,6-dione (IVa, 500 mg, 2.14 mmol) and (1R)-1-(4-methoxyphenyl)ethanamine (400 μL, 2.65 mmol), combined in 1,4-dioxane (5 mL). The mixture was stirred under nitrogen at 80° C. for 3 h. The reaction mixture was diluted with 5 mL of dioxane, then cooled to −12° C. and treated with sodium borohydride (162 mg, 4.29 mmol) in 10 mL anhydrous MeOH. The reaction mixture was stirred for 1 h, allowing the cooling bath to warm to 0° C. Stirring was continued for 30 min at 0° C. and the reaction was then quenched by the addition of 3 mL of brine and 15 mL of EtOAc at 0° C. The mixture was poured into a stirred mixture of 10 mL of brine and 40 mL of EtOAc and maintained at room temperature. After 15 min the mixture was filtered through CELITE®, and the filter cake was washed with an additional 40 mL of EtOAc. The combined filtrate was dried on sodium sulfate, filtered, and the solvent was evaporated under reduced pressure to provide a crude material as a mixture of diastereomers (S)-8-fluoro-1-(((R)-1-(4-methoxyphenyl)ethyl)amino)-1,5-dihydro-2H-pyrano[3,4-c]isoquinolin-6(4H)-one and (R)-8-fluoro-1-(((R)-1-(4-methoxyphenyl)ethyl)amino)-1,5-dihydro-2H-pyrano[3,4-c]isoquinolin-6(4H)-one, in a diastereomeric ratio (d.r.) ˜5:1 (by LCMS DAD integration). The major diastereoisomer was isolated by flash chromatography (silica gel, MeOH/DCM 0-2%, 15 min gradient, then 2% isocratic, MeOH/DCM) to afford (S)-8-fluoro-1-(((R)-1-(4-methoxyphenyl)ethyl)amino)-1,5-dihydro-2H-pyrano[3,4-c]isoquinolin-6(4H)-one (VIIIa, 522.0 mg, 66% yield; d.r.=49:1 by LCMS DAD integration). LCMS: m/z found 369.3 [M+H]⁺; RT=0.59, (Method B); ¹H NMR (400 MHz, CDCl₃) δ 11.12 (s, 1H), 8.04 (dd, 1H), 7.87 (dd, 1H), 7.47 (dddd, 1H), 7.34-7.23 (m, 2H), 6.92-6.80 (m, 2H), 4.63 (d, 1H), 4.56-4.47 (m, 1H), 4.17-4.03 (m, 2H), 3.87 (t, 1H), 3.78 (d, 3H), 3.52 (dd, 1H), 1.45 (dd, 3H).

### (S)-8-Fluoro-1-(((R)-1-(4-methoxyphenyl)ethyl)(methyl)amino)-1,5-dihydro-2H-pyrano[3,4-c]isoquinolin-6(4H)-one (IXa)

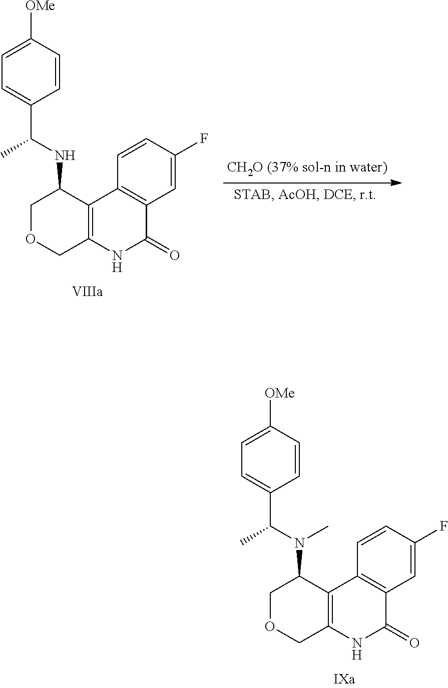 [SMILES: COc1ccc([C@@H](C)N[C@@H]2COCc3[nH]c(=O)c4cc(F)ccc4c32)cc1 ; COc1ccc([C@@H](C)N(C)[C@@H]2COCc3[nH]c(=O)c4cc(F)ccc4c32)cc1]

A mixture of enantiomerically pure (S)-8-fluoro-1-(((R)-1-(4-methoxyphenyl)ethyl)amino)-1,5-dihydro-2H-pyrano[3,4-c]isoquinolin-6(4H)-one (VIIIa, 120 mg, 0.33 mmol), an aqueous solution (37%) of formaldehyde (70 μL, 0.85 mmol), sodium triacetoxyborohydride (124 mg, 0.59 mmol), and acetic acid (34 μL, 0.59 mmol) were stirred in 1,2-dichloroethane (1.5 mL) overnight at room temperature. The reaction mixture was then diluted with 5 mL of dichloromethane and neutralized with 1 M aqueous NaOH. The aqueous phase was extracted with dichloromethane twice, and the combined organic extracts were washed with brine (1.5 mL), dried (sodium sulfate) and the solvent was evaporated under reduced pressure. The product was further purified by flash-chromatography (silica gel, EtOAc/hexanes) to provide enantiomerically pure (S)-8-fluoro-1-(((R)-1-(4-methoxyphenyl)ethyl)(methyl)amino)-1,5-dihydro-2H-pyrano[3,4-c]isoquinolin-6(4H)-one (IXa, 80.6 mg, 65%). LCMS: m/z found 383.3 [M+H]⁺; RT=2.09, (Method A); ¹H NMR (400 MHz, CDCl₃) δ 11.05 (s, 1H), 8.23 (dd, 1H), 8.05 (dd, 1H), 7.48 (ddd, 1H), 7.19-7.11 (m, 2H), 6.83-6.74 (m, 2H), 4.67 (d, 1H), 4.57-4.48 (m, 1H), 4.46 (d, 1H), 4.20 (s, 1H), 3.92 (q, 1H), 3.77 (s, 3H), 3.63 (dd, 1H), 2.16 (s, 3H), 1.50 (d, 3H).

### (S)-1-(Ethyl((R)-1-(4-methoxyphenyl)ethyl)amino)-8-fluoro-1,5-dihydro-2H-pyrano[3,4-c]isoquinolin-6(4H)-one (IXb)

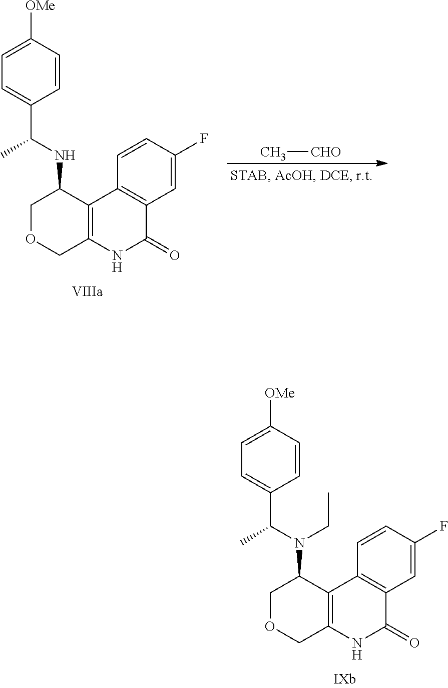

Enantiomerically pure (S)-1-(ethyl((R)-1-(4-methoxyphenyl)ethyl)amino)-8-fluoro-1,5-dihydro-2H-pyrano[3,4-c]isoquinolin-6(4H)-one was synthesized in an analogous manner as described above for IXa, in 86% yield, starting from enantiomerically pure (S)-8-fluoro-1-(((R)-1-(4-methoxyphenyl)ethyl)amino)-1,5-dihydro-2H-pyrano[3,4-c]isoquinolin-6(4H)-one (VIIIa) and acetaldehyde. LCMS m/z found 397.4 [M+H]⁺; RT=0.64 min (Method B); ¹H NMR (400 MHz, CDCl₃) δ 12.03 (s, 1H), 8.11 (dd, 1H), 8.02 (dd, 1H), 7.39 (ddd, 1H), 7.03 (d, 2H), 6.74-6.65 (m, 2H), 4.77 (d, 1H), 4.64-4.49 (m, 2H), 4.19-4.06 (m, 2H), 3.74 (s, 3H), 3.66 (dd, 1H), 2.83 (dq, 1H), 2.72 (dq, 1H), 1.48 (d, 3H), 0.90 (t, 3H).

### (S)-8,9-Difluoro-1-(((R)-1-(4-methoxyphenyl)ethyl)amino)-1,5-dihydro-2H-pyrano[3,4-c]isoquinolin-6(4H)-one (VIIIb)

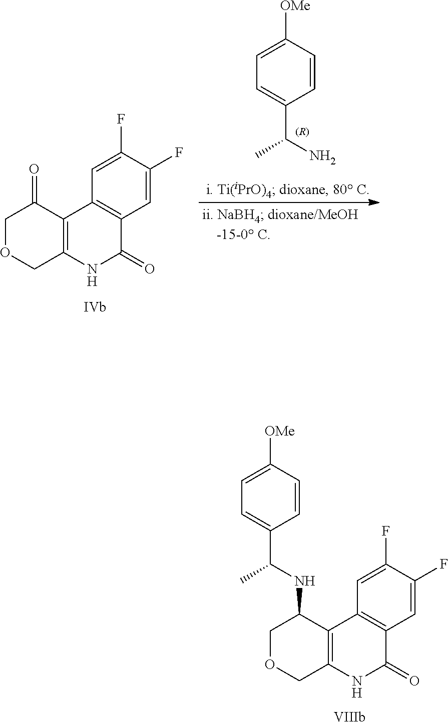

Enantiomerically pure (S)-8,9-difluoro-1-(((R)-1-(4-methoxyphenyl)ethyl)amino)-1,5-dihydro-2H-pyrano[3,4-c]isoquinolin-6(4H)-one was synthesized in an analogous manner as described above for VIIIa, in 69% yield, starting from 8,9-difluoro-2H-pyrano[3,4-c]isoquinoline-1,6(4H,5H)-dione (IVb) and (1R)-1-(4-methoxyphenyl)ethanamine. LCMS m/z found 387.27 [M+H]⁺; RT=0.60 min (Method B); ¹H NMR (400 MHz, CDCl₃) δ 11.29 (bs, 1H), 8.14 (dd, 1H), 7.67 (dd, 1H), 7.31-7.27 (m, 2H), 6.90-6.79 (m, 2H), 4.61 (d, 1H), 4.55-4.46 (m, 1H), 4.23-4.15 (m, 1H), 4.08 (q, 1H), 3.84-3.76 (m, 1H), 3.78 (s, 3H), 3.54 (dd, 1H), 1.75 (bs, 1H), 1.47 (d, 3H). The absolute configuration of VIIIb was unambiguously determined by X-ray crystallography.

### X-Ray Structure Determination of VIIb

Crystals of VIIIb were grown by vapor diffusion, using dichloromethane as the solvent and 1:2 v/v diethyl ether:hexanes as the anti-solvent. VIIIb (Molecular formula: C₂₁H₂₀F₂N₂O₃) crystallizes in the monoclinic space group C2 (systematic absences hkl: h+k=odd) with a=16.6854(3)Å, b=7.42270(10)Å, c=30.0803(5)Å, β=93.8300(10)°, V=3717.15(10)Å³, Z=8, and d_{calc}=1.381 g/cm³. X-ray intensity data were collected on a Rigaku XtaLAB Synergy-S diffractometer [1] equipped with an HPC area detector (HyPix-6000HE) and employing confocal multilayer optic-monochromated Cu-Kα radiation (λ=1.54184 Å) at a temperature of 100 K. Preliminary indexing was performed from a series of sixty 0.5° rotation frames with exposures of 0.5 seconds for θ=±47.199° and 2 seconds for θ=107.75°. A total of 6758 frames (53 runs) were collected employing CO scans with a crystal to detector distance of 34.0 mm, rotation widths of 0.5° and exposures of 0.5 seconds for θ=±47.199° and 2 seconds for θ=107.75° and −86.25°.

Rotation frames were integrated using CrysAlisPro [2], producing a listing of unaveraged F² and σ(F²) values. A total of 42362 reflections were measured over the ranges 5.89≤2θ≤148.978°, −19≤h≤20, −9≤k≤9, −37≤1≤37 yielding 7486 unique reflections (Rᵢₙₜ=0.0573). The intensity data were corrected for Lorentz and polarization effects and for absorption using SCALE3 ABSPACK [3] (minimum and maximum transmission 0.5308, 1.0000). The structure was solved by direct methods—SHELXT [4]. There are two crystallographically-independent molecules in the asymmetric unit. Refinement was by full-matrix least squares based on F² using SHELXL-2018 [5]. All reflections were used during refinement. The weighting scheme used was w=1/[σ²(Fₒ²)+(0.0530P)²+10.5599P] where P=(Fₒ²+2F_{c}²)/3. Non-hydrogen atoms were refined anisotropically and hydrogen atoms were refined using a riding model. Refinement converged to R1=0.0573 and wR2=0.1568 for 7232 observed reflections for which F>4σ(F) and R1=0.0585 and wR2=0.1575 and GOF=1.136 for all 7486 unique, non-zero reflections and 509 variables. The maximum Δ/σ in the final cycle of least squares was 0.001 and the two most prominent peaks in the final difference Fourier were +0.44 and −0.30 e/Å³. The Hooft absolute structure parameter y [6] was calculated using PLATON [7] The resulting value was y=0.03(4) indicating that the absolute structure has been assigned correctly. The Flack parameter [7] refined to a similar value of 0.03(5). If these parameters are equal to 0 (within 3 standard deviations) then the absolute structure has been assigned correctly; if they are 1, the opposite enantiomer has been modeled.

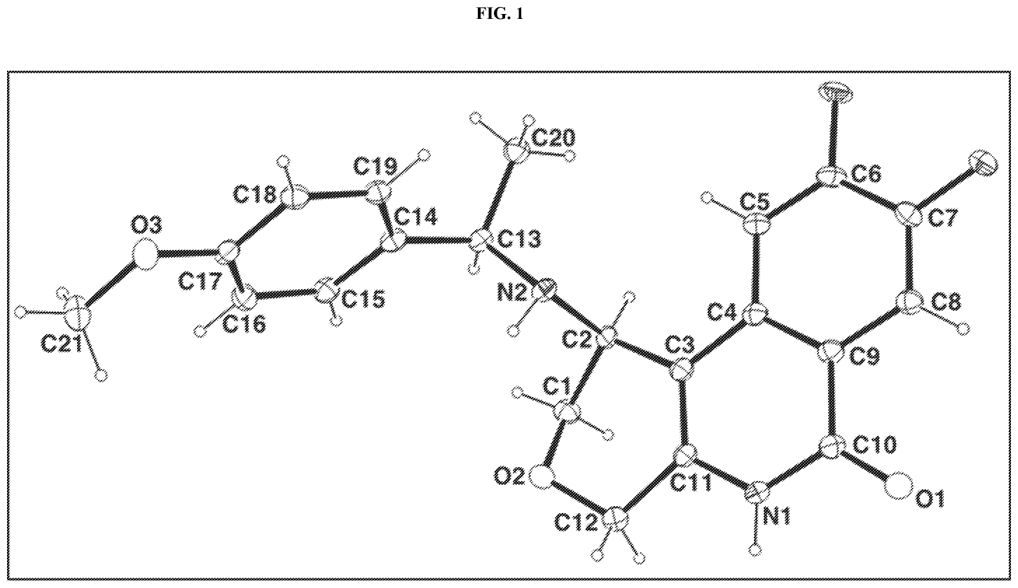

Table 1 lists cell information, data collection parameters, and refinement data for VIIIb, whereas final positional and equivalent thermal parameters for VIIIb are provided in Table 2. FIG. 1 provides the ORTEP representation of VIIIb with 50% probability thermal ellipsoids displayed, defining the absolute configuration of VIIIb as (S)-8,9-Difluoro-1-(((R)-1-(4-methoxyphenyl)ethyl)amino)-1,5-dihydro-2H-pyrano[3,4-c]isoquinolin-6(4H)-one.

TABLE 1
Summary of structure determination of VIIIb

| Empirical formula | C₂₁H₂₀F₂N₂O₃ |
|---|---|
| Formula weight | 386.39 |
| Diffractometer | Rigaku XtaLAB Synergy-S (HyPix-6000HE) |
| Temperature/K | 100 |
| Crystal system | monoclinic |
| Space group | C2 |
| a | 16.6854(3)Å |
| b | 7.42270(10)Å |
| c | 30.0803(5)Å |
| β | 93.8300(10)° |
| Volume | 3717.15(10)Å³ |
| Z | 8 |
| d_{calc} | 1.381 g/cm³ |
| μ | 0.894 mm⁻¹ |
| F(000) | 1616.0 |
| Crystal size, mm | 0.36 × 0.26 × 0.06 |
| 2θ range for data collection | 5.89-148.978° |
| Index ranges | −19 ≤ h ≤ 20, −9 ≤ k ≤ 9, −37 ≤ 1 ≤ 37 |
| Reflections collected | 42362 |
| Independent reflections | 7486[R(int) = 0.0573] |
| Data/restraints/parameters | 7486/1/509 |
| Goodness-of-fit on F² | 1.136 |
| Final R indexes [I >= 2σ | R₁ = 0.0573, wR₂ = 0.1568 |
| (I)] | |
| Final R indexes [all data] | R₁ = 0.0585, wR₂ = 0.1575 |
| Largest diff. peak/hole | 0.44/−0.30 eÅ⁻³ |
| Hooft parameter | 0.03(4) |
| Flack parameter | 0.03(5) |

TABLE 2
Refined positional parameters for VIIIb

| Atom | x | y | z | U(eq) |
|---|---|---|---|---|
| F1 | 0.53964(16) | 0.9183(5) | 0.28261(10) | 0.0414(8) |
| F2 | 0.64570(17) | 0.9539(4) | 0.35198(9) | 0.0335(6) |
| O1 | 0.91635(19) | 0.8385(5) | 0.29191(11) | 0.0350(8) |
| O2 | 0.81651(18) | 0.7785(5) | 0.09999(10) | 0.0284(7) |
| O3 | 0.6019(2) | −0.0081(4) | 0.01054(11) | 0.0316(8) |
| N1 | 0.8876(2) | 0.8172(6) | 0.21736(12) | 0.0268(8) |
| N2 | 0.6610(2) | 0.6667(5) | 0.13236(12) | 0.0236(8) |
| C1 | 0.7503(3) | 0.9006(7) | 0.10239(15) | 0.028(1) |
| C2 | 0.7005(3) | 0.8421(6) | 0.14046(14) | 0.0225(9) |
| C3 | 0.7548(3) | 0.8332(6) | 0.18293(14) | 0.0257(9) |
| C4 | 0.7241(2) | 0.8526(6) | 0.22650(15) | 0.0216(8) |
| C5 | 0.6412(3) | 0.8681(6) | 0.23338(15) | 0.0270(9) |
| C6 | 0.6186(3) | 0.8990(7) | 0.27511(16) | 0.0289(10) |
| C7 | 0.6727(3) | 0.9165(7) | 0.31171(15) | 0.0274(10) |
| C8 | 0.7536(3) | 0.8962(6) | 0.30678(15) | 0.0260(9) |
| C9 | 0.7797(3) | 0.8623(6) | 0.26428(15) | 0.0243(9) |
| C10 | 0.8651(3) | 0.8407(7) | 0.25954(15) | 0.0277(9) |
| C11 | 0.8340(3) | 0.8166(6) | 0.18008(15) | 0.0239(9) |
| C12 | 0.8740(3) | 0.8017(7) | 0.13666(14) | 0.0288(10) |
| C13 | 0.5931(3) | 0.6696(6) | 0.09788(14) | 0.0230(9) |
| C14 | 0.5911(3) | 0.4920(6) | 0.07310(15) | 0.0241(9) |
| C15 | 0.6157(3) | 0.4813(6) | 0.03014(16) | 0.0294(10) |
| C16 | 0.6197(3) | 0.3177(7) | 0.00738(15) | 0.0280(9) |
| C17 | 0.5987(3) | 0.1618(6) | 0.02841(15) | 0.0256(9) |
| C18 | 0.5716(3) | 0.1693(6) | 0.07129(16) | 0.0279(10) |
| C19 | 0.5684(3) | 0.3320(6) | 0.09336(15) | 0.0245(9) |
| C20 | 0.5155(3) | 0.7111(7) | 0.12072(16) | 0.0296(10) |
| C21 | 0.6330(4) | −0.0232(7) | −0.03217(16) | 0.0379(12) |

### (S)-8,9-Difluoro-1-(((R)-1-(4-methoxyphenyl)ethyl)(methyl)amino)-1,5-dihydro-2H-pyrano[3,4-c]isoquinolin-6(4H)-one (MX)

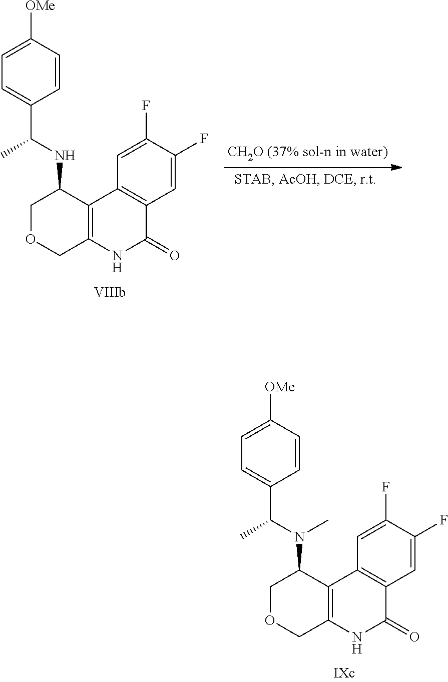 [SMILES: COc1ccc([C@@H](C)N[C@@H]2COCc3[nH]c(=O)c4cc(F)c(F)cc4c32)cc1 ; COc1ccc([C@@H](C)N(C)[C@@H]2COCc3[nH]c(=O)c4cc(F)c(F)cc4c32)cc1]

Enantiomerically pure (S)-8,9-difluoro-1-(((R)-1-(4-methoxyphenyl)ethyl)(methyl)amino)-1,5-dihydro-2H-pyrano[3,4-c]isoquinolin-6(4H)-one was synthesized in an analogous manner as described above for IXa, in 82% yield, starting from enantiomerically pure (S)-8,9-difluoro-1-(((R)-1-(4-methoxyphenyl)ethyl)amino)-1,5-dihydro-2H-pyrano[3,4-c]isoquinolin-6(4H)-one (VIIIb). LCMS m/z found 401.3 [M+H]⁺; RT=2.24 min (Method A); ¹H NMR (400 MHz, CDCl₃) δ 12.01 (s, 1H), 8.14 (dd, 1H), 8.03 (dd, 1H), 7.22-7.10 (m, 2H), 6.85-6.74 (m, 2H), 4.70 (d, 1H), 4.53 (dd, 1H), 4.45 (d, 1H), 4.19-4.06 (m, 1H), 3.90 (q, 1H), 3.78 (s, 3H), 3.63 (dd, 1H), 2.14 (s, 3H), 1.51 (d, 3H).

### (S)-8,9-Difluoro-1-(methylamino)-1,5-dihydro-2H-pyrano[3,4-c]isoquinolin-6(4H)-one

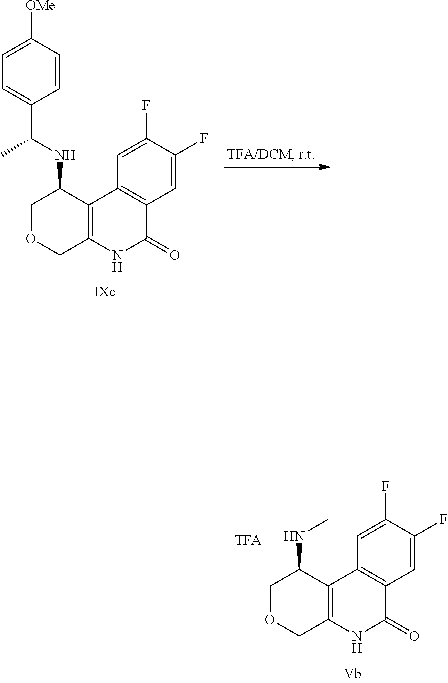 [SMILES: COc1ccc([C@@H](C)N[C@@H]2COCc3[nH]c(=O)c4cc(F)c(F)cc4c32)cc1 ; CN[C@@H]1COCc2[nH]c(=O)c3cc(F)c(F)cc3c21]

(S)-8,9-Difluoro-1-(((R)-1-(4-methoxyphenyl)ethyl)(methyl)amino)-1,5-dihydro-2H-pyrano[3,4-c]isoquinolin-6(4H)-one (IXc, 1.93 g, 4.82 mmol) was stirred overnight with trifluoroacetic acid (20 mL, 175.4 mmol) in dichloromethane (20.0 mL) at room temperature, under nitrogen. The reaction mixture was then treated with 40 mL of MeOH and the mixture stirred for 20 min, when the deep purple, opaque mixture transitioned to a yellow, transparent solution. The volatiles were evaporated, and the residue was dried further by azeotropic evaporation with a 1:1 v/v methanol/toluene mixture, then once with toluene. Trituration with diethyl ether for 15 min generated a precipitate that was collected by filtration, washed with diethyl ether, and dried under high vacuum to provide enantiomerically pure (S)-8,9-difluoro-1-(methylamino)-1,5-dihydro-2H-pyrano[3,4-c]isoquinolin-6(4H)-one as a mono-trifluoroacetate salt (1.67 g, 91%). LCMS found m/z 267.2 [M+H]⁺; RT=0.47 min (Method B); ¹H NMR (400 MHz, Methanol-d₄) δ 8.17 (dd, 1H), 7.83 (dd, 1H), 4.89 (s, 1H), 4.76-4.60 (m, 2H), 4.58 (s, 1H), 4.51 (dd, 1H), 3.98 (dd, 1H), 2.86 (s, 3H). A portion of the TFA salt of Vb, obtained as above, was partitioned between ethyl acetate and saturated sodium bicarbonate. The aqueous phase was further extracted with ethyl acetate, ensuring a pH >8.5 after the final extraction, and the combined organic extracts were dried over sodium sulfate, filtered, the solvent was evaporated under reduced pressure and the solid residue was further dried under high vacuum to afford enantiomerically pure (S)-8,9-difluoro-1-(methylamino)-1,5-dihydro-2H-pyrano[3,4-c]isoquinolin-6(4H)-one (Vb) as a free base. ¹H NMR (400 MHz, DMSO-d₆) δ 11.40 (br s, 1H), 8.03 (dd, 1H), 7.73 (dd, 1H), 4.41 (d, 1H), 4.34 (d, 1H), 4.22 (dd, 1H), 3.59-3.51 (m, 1H), 3.33 (s, 1H), 2.39 (s, 3H), 1.90 (br s, 1H).

### (S)-6-Chloro-N-(8,9-difluoro-6-oxo-1,4,5,6-tetrahydro-2H-pyrano[3,4-c]isoquinolin-1-yl)-N-methylimidazo[1,2-a]pyridine-2-carboxamide (Compound 51)

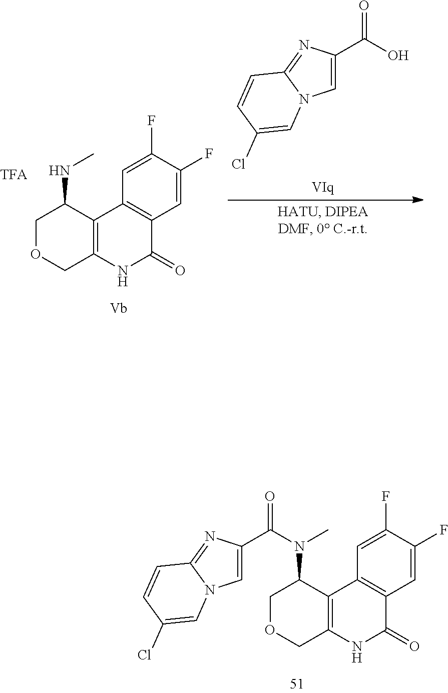

A stirred solution of 6-chloroimidazo[1,2-a]pyridine-2-carboxylic acid (VIq, 39 mg, 0.20 mmol) in DMF (1 mL) was treated with DIPEA (125 μL, 0.71 mmol) and HATU (81 mg, 0.21 mmol) at room temperature for 10 min. Enantiomerically pure (S)-8,9-difluoro-1-(methylamino)-1,5-dihydro-2H-pyrano[3,4-c]isoquinolin-6(4H)-one, (Vb, 70 mg TFA salt, 0.19 mmol) was added next and the reaction was continued for 2 h. The reaction was quenched by addition of 5 mL of saturated sodium bicarbonate, and then extracted twice with ethyl acetate (30 mL each). The combined organic extracts were washed with brine once (10 mL), dried over sodium sulfate, filtered, and the solvent was evaporated under reduced pressure. The product was isolated by flash chromatography (silicagel, ethyl acetate/hexanes 0-100% for 5 min, then isocratic 100% ethyl acetate) to afford enantiomerically pure (S)-6-chloro-N-(8,9-difluoro-6-oxo-1,4,5,6-tetrahydro-2H-pyrano[3,4-c]isoquinolin-1-yl)-N-methylimidazo[1,2-a]pyridine-2-carboxamide (46 mg, 55%): LCMS m/z found 445.2/447.1 [M+H]⁺; RT=2.83 min (Method A); ¹H NMR (400 MHz, DMSO-d₆) δ 11.70 (d, 1H), 8.94 (dd)*, 8.86 (dd, 1H), 8.46 (d)*, 8.40 (d, 1H), 8.11 (ddd, 1H), 7.76 (dd)*, 7.73-7.62 (m, 1H), 7.45-7.33 (m)*, 6.37 (s)*, 5.72 (s, 1H), 4.61 (dd, 1H), 4.45 (t, 1H), 4.20-4.12 (m, 1H), 4.01 (ddd, 1H), 3.22 (s, 3H), 2.80 (s).* (NOTE: “*” denotes observed signals of minor amide rotamer).

### (S)-N-(8,9-Difluoro-6-oxo-1,4,5,6-tetrahydro-2H-pyrano[3,4-c]isoquinolin-1-yl)-5,6-difluoro-N-methyl-1H-indole-2-carboxamide (Compound 42)

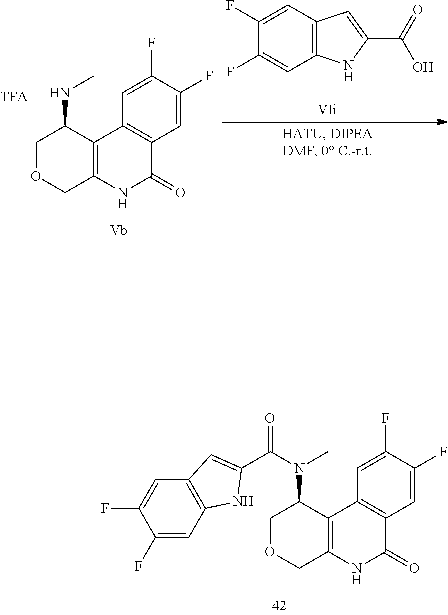

Enantiomerically pure (S)-N-(8,9-difluoro-6-oxo-1,4,5,6-tetrahydro-2H-pyrano[3,4-c]isoquinolin-1-yl)-5,6-difluoro-N-methyl-1H-indole-2-carboxamide was synthesized in an analogous manner as described above (Compound 51) from enantiomerically pure (S)-8,9-difluoro-1-(methylamino)-1,5-dihydro-2H-pyrano[3,4-c]isoquinolin-6(4H)-one (Vb) and 5,6-difluoro-1H-indole-2-carboxylic acid (Vli). LCMS: m/z found 446.2 [M+H]⁺, RT=5.08 min, (Method A); ¹H NMR (400 MHz, DMSO-d₆) δ 11.92 (br s, 1H, exch.), 11.74 (br s, 1H, exch.), 8.12 (dd, 1H), 7.61 (dd, 1H), 7.41 (m, 2H), 6.97 (s, 1H), 5.75 (s, 1H), 4.64 (d, 1H), 4.53-4.43 (m, 1H), 4.17 (d, 1H), 4.09-3.99 (m, 1H), 3.15 (s, 3H).

### (S)-6-Chloro-N-(8-fluoro-6-oxo-1,4,5,6-tetrahydro-2H-pyrano[3,4-c]isoquinolin-1-yl)-N-methylimidazo[1,2-a]pyridine-2-carboxamide (Compound 52)

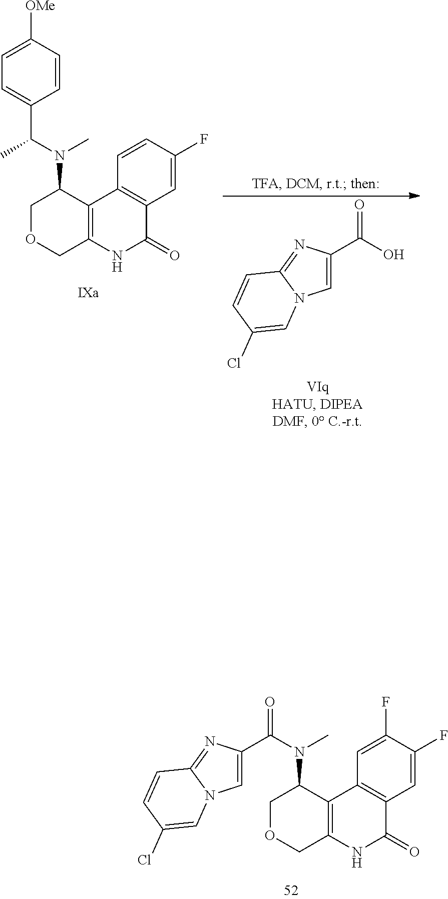 [SMILES: COc1ccc([C@@H](C)N(C)[C@@H]2COCc3[nH]c(=O)c4cc(F)ccc4c32)cc1 ; O=C(O)c1cn2cc(Cl)ccc2n1 ; CN(C(=O)c1cn2cc(Cl)ccc2n1)[C@@H]1COCc2[nH]c(=O)c3cc(F)c(F)cc3c21]

Enantiomerically pure (S)-6-chloro-N-(8-fluoro-6-oxo-1,4,5,6-tetrahydro-2H-pyrano[3,4-c]isoquinolin-1-yl)-N-methylimidazo[1,2-a]pyridine-2-carboxamide was synthesized in an analogous manner as described above from enantiomerically (S)-8-fluoro-1-(((R)-1-(4-methoxyphenyl)ethyl)(methyl)amino)-1,5-dihydro-2H-pyrano[3,4-c]isoquinolin-6(4H)-one (IXa) and 6-chloroimidazo[1,2-a]pyridine-2-carboxylic acid (VIq). LCMS m/z found 427.2/428.8 [M+H]⁺; RT=2.56 min (Method A); ¹H NMR (400 MHz, Methanol-d₄) δ 8.69 (ddd, 1H), 8.41 (d)*, 8.28-8.23 (m, 1H), 8.02-7.92 (m, 1H), 7.70 (dt, 1H), 7.66-7.43 (m, 2H), 7.38 (dt, 1H), 6.40 (s)*, 5.86 (s, 1H), 4.68 (d, 1H), 4.62-4.49 (m, 1H), 4.45 (d)*, 4.39-4.30 (m, 1H), 4.10 (ddd, 1H), 3.20 (s, 3H), 2.95 (s)*. (NOTE: “*” denotes observed signals belonging to minor amide rotamer).

### (S)-6-Chloro-N-ethyl-N-(8-fluoro-6-oxo-1,4,5,6-tetrahydro-2H-pyrano[3,4-c]isoquinolin-1-yl)imidazo[1,2-a]pyridine-2-carboxamide (Compound 53)

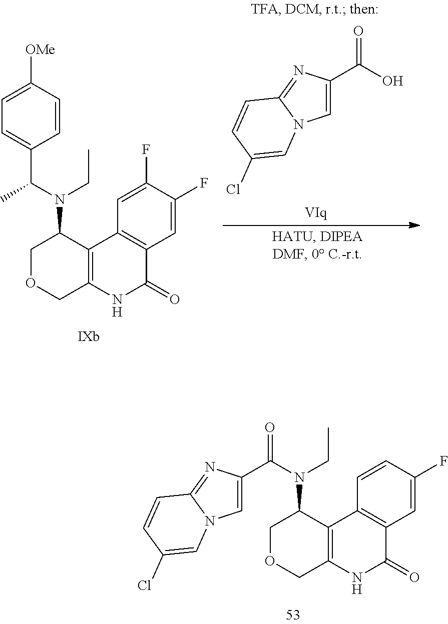

Enantiomerically pure (S)-6-chloro-N-ethyl-N-(8-fluoro-6-oxo-1,4,5,6-tetrahydro-2H-pyrano[3,4-c]isoquinolin-1-yl)imidazo[1,2-a]pyridine-2-carboxamide was synthesized in an analogous manner as described above from enantiomerically pure (S)-1-(ethyl((R)-1-(4-methoxyphenyl)ethyl)amino)-8-fluoro-1,5-dihydro-2H-pyrano[3,4-c]isoquinolin-6(4H)-one (IXb) and 6-chloroimidazo[1,2-a]pyridine-2-carboxylic acid (VIq). LCMS m/z found 441.2 [M+H]⁺; RT=2.93 min (Method A); ¹H NMR (400 MHz, Methanol-d₄) δ 8.74-8.69 (m, 1H)*, 8.69-8.65 (m, 1H)*, 8.39 (s, 1H), 8.26 (s, 1H), 7.96 (ddd, 2H, overlapped signals of two rotamers), 7.74-7.43 (m, 6H, overlapped signals of two rotamers), 7.38 (dd, 1H), 7.35 (dd)*, 6.36 (s)*, 5.90 (s, 1H), 4.69 (d, 2H, overlapped signals of two rotamers), 4.55 (t, 2H, overlapped signals of two rotamers), 4.43 (d, 1H)*, 4.29 (d, 1H), 4.12-3.97 (m, 3H, overlapped signals of two rotamers), 3.71-3.41 (m, 3H, overlapped signals of two rotamers), 0.94 (t, 3H)*, 0.82 (t, 3H). (NOTE: “*” denotes distinguishable signals of minor rotamer in a nearly 1:1 mixture.)

### (S)-N-(8,9-Difluoro-6-oxo-1,4,5,6-tetrahydro-2H-pyrano[3,4-c]isoquinolin-1-yl)-N-methylpyrazolo[1,5-a]pyridine-2-carboxamide (Compound 64)

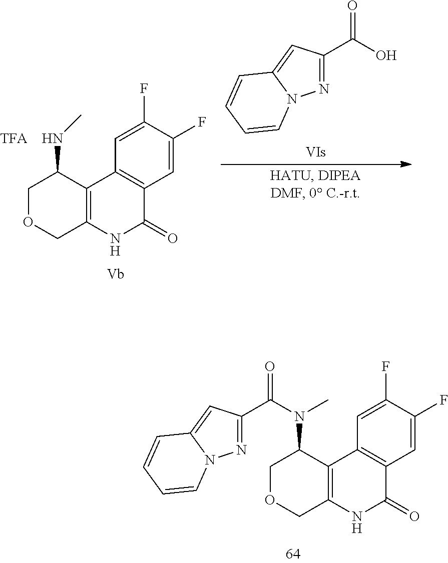

Enantiomerically pure (S)-N-(8,9-difluoro-6-oxo-1,4,5,6-tetrahydro-2H-pyrano[3,4-c]isoquinolin-1-yl)-N-methylpyrazolo[1,5-a]pyridine-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and pyrazolo[1,5-a]pyridine-2-carboxylic acid (VIs). LCMS m/z found 411.2 [M+H]⁺; RT=3.28 min (Method A); ¹H NMR (400 MHz, Chloroform-d) δ 12.35 (s, 1H), 8.42 (dq, 1H), 8.20 (dt, 1H), 7.69-7.48 (m, 2H), 7.33-7.10 (m, 2H), 6.97-6.79 (m, 2H), 6.09 (s*), 5.97-5.91 (m, 1H), 4.85 (dd, 1H), 4.72-4.59 (m, 1H), 4.51-4.40 (m, 1H), 4.17-4.00 (m, 1H), 3.21 (s, 3H), 3.01 (s)*. (NOTE: “*” denotes observed signals of minor amide rotamer.)

### (S)-N-(8,9-Difluoro-6-oxo-1,4,5,6-tetrahydro-2H-pyrano[3,4-c]isoquinolin-1-yl)-N-methylpyrrolo[1,2-b]pyridazine-6-carboxamide (Compound 65)

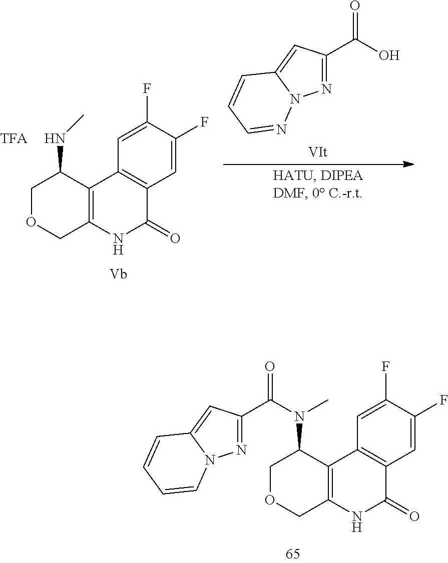

Enantiomerically pure (S)-N-(8,9-difluoro-6-oxo-1,4,5,6-tetrahydro-2H-pyrano[3,4-c]isoquinolin-1-yl)-N-methylpyrrolo[1,2-b]pyridazine-6-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and pyrrolo[1,2-b]pyridazine-6-carboxylic acid (VIt). LCMS m/z found 411.2 [M+H]⁺; RT=3.35 min (Method A); ¹H NMR (400 MHz, Chloroform-d) δ 11.70 (s, 1H), 8.20 (dd, 1H), 8.13-7.97 (m, 2H), 7.73 (dd, 1H), 7.49 (dd, 1H), 6.74 (d, 1H), 6.57 (dd, 1H), 5.88 (d, 1H), 4.78 (d, 1H), 4.67-4.57 (m, 1H), 4.40 (d, 1H), 4.07 (dd, 1H), 3.13 (s, 3H).

### (S)-N-(8,9-Difluoro-6-oxo-1,4,5,6-tetrahydro-2H-pyrano[3,4-c]isoquinolin-1-yl)-N-methylquinoline-7-carboxamide (Compound 66)

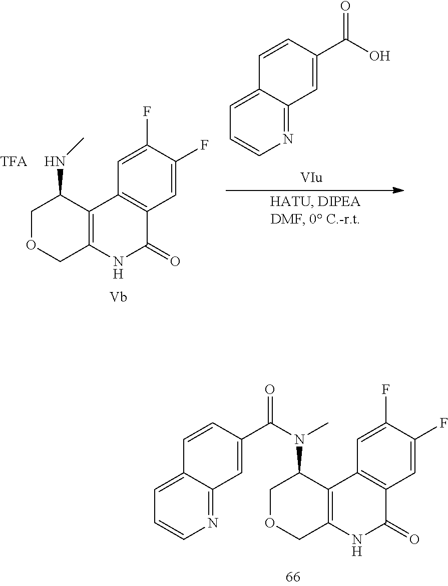

Enantiomerically pure (S)-N-(8,9-difluoro-6-oxo-1,4,5,6-tetrahydro-2H-pyrano[3,4-c]isoquinolin-1-yl)-N-methylquinoline-7-carboxamide was synthesized in an analogous manner as described above, from enantiomerically pure (S)-8,9-difluoro-1-(methylamino)-1,5-dihydro-2H-pyrano[3,4-c]isoquinolin-6(4H)-one (Vb) and quinoline-7-carboxylic acid (VIu). LCMS m/z found 422.2 [M+H]⁺; RT=2.30 min (Method A); ¹H NMR (400 MHz, Chloroform-d) δ 12.29 (s, 1H), 8.96 (dd, 1H), 8.28-8.14 (m, 2H), 8.15-8.00 (m, 1H), 7.92 (d, 1H), 7.67-7.56 (m, 2H), 7.47 (dd, 1H), 5.96-5.91 (m, 1H), 4.83 (d, 1H), 4.72-4.63 (m, 1H), 4.53-4.45 (m, 1H), 4.18-4.06 (m, 1H), 2.92 (s, 3H).

### (S)-N-(8,9-Difluoro-6-oxo-1,4,5,6-tetrahydro-2H-pyrano[3,4-c]isoquinolin-1-yl)-N-methylquinoline-6-carboxamide (Compound 67)

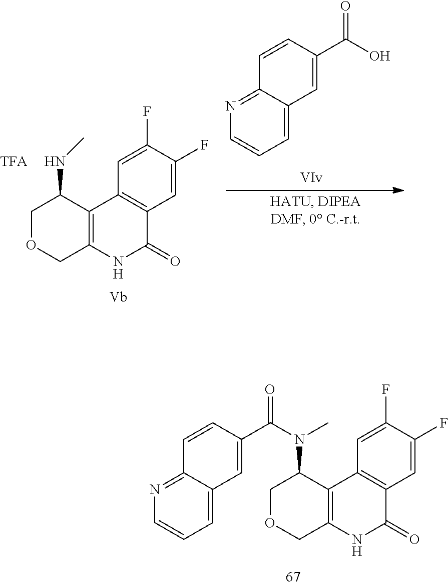

Enantiomerically pure (S)-N-(8,9-difluoro-6-oxo-1,4,5,6-tetrahydro-2H-pyrano[3,4-c]isoquinolin-1-yl)-N-methylquinoline-6-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and quinoline-6-carboxylic acid (VIv). LCMS m/z found 422.2 [M+H]⁺; RT=2.19 min (Method A); ¹H NMR (400 MHz, Chloroform-d) δ 12.29 (s, 1H), 8.98 (dd, 1H), 8.30-8.13 (m, 3H), 7.92 (d, 1H), 7.72 (dd, 1H), 7.62 (dd, 1H), 7.47 (dd, 1H), 5.97-5.91 (m, 1H), 4.84 (d, 1H), 4.74-4.64 (m, 1H), 4.49 (d, 1H), 4.13 (dd, 1H), 2.89 (s, 3H).

### (S)-N-(8,9-Difluoro-6-oxo-1,4,5,6-tetrahydro-2H-pyrano[3,4-c]isoquinolin-1-yl)-N-methyl-[1,2,4]triazolo[4,3-a]pyridine-6-carboxamide (Compound 70)

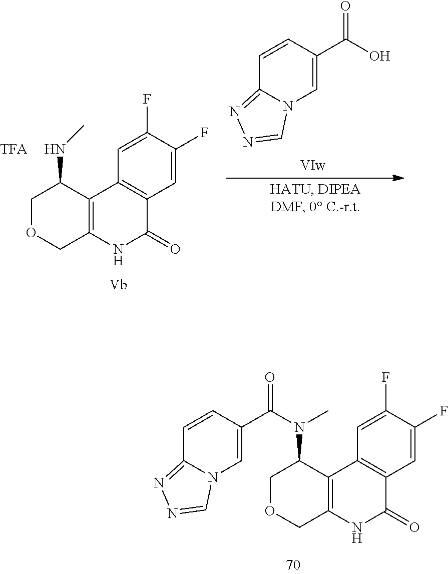

Enantiomerically pure (S)-N-(8,9-difluoro-6-oxo-1,4,5,6-tetrahydro-2H-pyrano[3,4-c]isoquinolin-1-yl)-N-methyl-[1,2,4]triazolo[4,3-a]pyridine-6-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and [1,2,4]triazolo[4,3-a]pyridine-6-carboxylic acid (VIw). LCMS m/z found 412 [M+H]⁺; RT=2.08 min (Method C); ¹H NMR (400 MHz, Methanol-d₄) δ 9.22 (d, 1H), 8.81-8.75 (m, 1H), 8.17 (dd, 1H), 7.83 (d, 1H), 7.59-7.49 (m, 2H), 5.81 (d, 1H), 4.68 (d, 1H), 4.57 (d, 1H), 4.41 (d, 1H), 4.12 (dd, 1H), 2.98 (s, 3H).

### (S)-N-(8,9-Difluoro-6-oxo-1,4,5,6-tetrahydro-2H-pyrano[3,4-c]isoquinolin-1-yl)-N-methylquinoline-3-carboxamide (Compound 71)

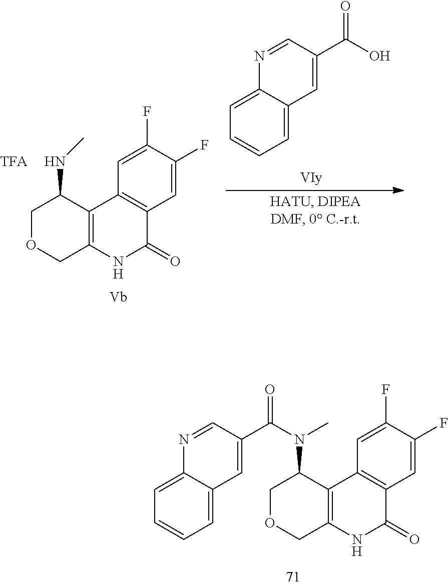

Enantiomerically pure (S)-N-(8,9-difluoro-6-oxo-1,4,5,6-tetrahydro-2H-pyrano[3,4-c]isoquinolin-1-yl)-N-methylquinoline-3-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and quinoline-3-carboxylic acid (VIy). LCMS m/z found 422.2 [M+H]⁺; RT=3.16 min (Method A); ¹H NMR (400 MHz, Chloroform-d) δ 12.42-12.37 (m, 1H), 8.96 (d, 1H), 8.30-8.20 (m, 2H), 8.17-8.10 (m, 1H), 7.91-7.84 (m, 1H), 7.79 (ddd, 1H), 7.66-7.55 (m, 2H), 5.96-5.90 (m, 1H), 4.85 (d, 1H), 4.75-4.65 (m, 1H), 4.54-4.46 (m, 1H), 4.19-4.06 (m, 1H), 2.95 (s, 3H).

### (S)-N-(8,9-Difluoro-6-oxo-1,4,5,6-tetrahydro-2H-pyrano[3,4-c]isoquinolin-1-yl)-N-methylquinoxaline-6-carboxamide (Compound 72)

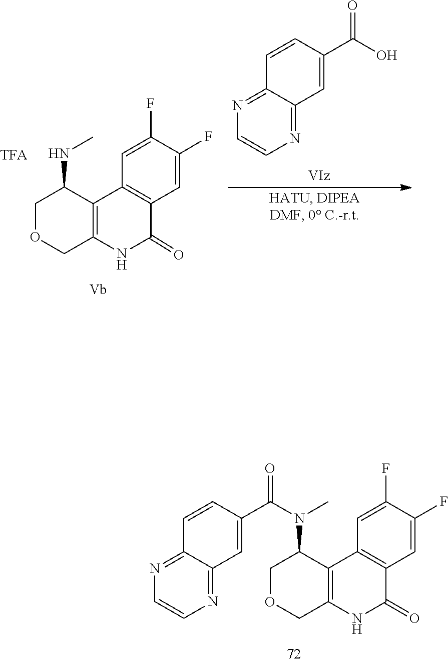

Enantiomerically pure (S)-N-(8,9-difluoro-6-oxo-1,4,5,6-tetrahydro-2H-pyrano[3,4-c]isoquinolin-1-yl)-N-methylquinoxaline-6-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and quinoxaline-6-carboxylic acid (VIz). LCMS m/z found 423 [M+H]⁺; RT=2.42 min (Method C); ¹H NMR (400 MHz, Chloroform-d) δ 12.35 (s, 1H), 8.90 (q, 2H), 8.30-8.16 (m, 2H), 8.13 (d, 1H), 7.83 (dd, 1H), 7.60 (dd, 1H), 5.97-5.91 (m, 1H), 4.85 (d, 1H), 4.74-4.62 (m, 1H), 4.49 (d, 1H), 4.19-4.06 (m, 1H), 2.91 (s, 3H).

### (S)-N-(8,9-Difluoro-6-oxo-1,4,5,6-tetrahydro-2H-pyrano[3,4-c]isoquinolin-1-yl)-N-methyl-6-(trifluoromethyl)nicotinamide (Compound 73)

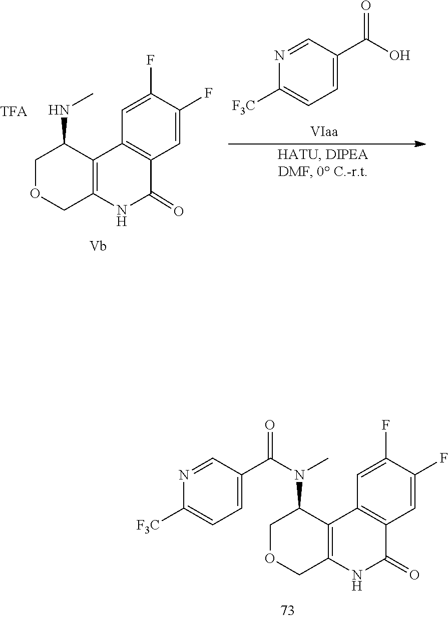

A stirred solution of 6-(trifluoromethyl)pyridine-3-carboxylic acid (VIaa, 32 mg, 0.17 mmol) in DMF (1 mL) was treated with DIEA (105 uL, 0.60 mmol) and HATU (114 mg, 0.30 mmol) at room temperature for 10 min. Enantiomerically pure (S)-8,9-difluoro-1-(methylamino)-1,5-dihydro-2H-pyrano[3,4-c]isoquinolin-6(4H)-one (Vb, 56 mg TFA salt, 0.15 mmol) was added and stirring was continued for 2 h. The reaction was quenched by addition of 5 mL of saturated ammonium chloride (pH adjusted to ˜4 by the dropwise addition of 2 M HCl), and then extracted twice with ethyl acetate (30 mL each). The combined organic extracts were washed twice with 25 mL each of saturated ammonium chloride (pH adjusted to ˜4 by the dropwise addition of 2 M HCl), followed by twice with 25 mL each of saturated sodium bicarbonate, then once with water (25 mL), and once with brine (20 mL), dried over sodium sulfate, filtered and the solvent evaporated. The product was isolated by flash chromatography (silicagel, methanol/dichloromethane 0-10% over 20 min) to afford (S)-N-(8,9-difluoro-6-oxo-1,4,5,6-tetrahydro-2H-pyrano[3,4-c]isoquinolin-1-yl)-N-methyl-6-(trifluoromethyl)nicotinamide (yield: 38 mg, 58%). LCMS m/z found 440.1 [M+H]⁺; RT=3.95 min (Method A); ¹H NMR (400 MHz, Chloroform-d) δ 12.45-12.40 (m, 1H), 8.80-8.75 (m, 1H), 8.24 (dd, 1H), 8.00-7.92 (m, 1H), 7.78 (dd, 1H), 7.48 (dd, 1H), 5.90-5.84 (m, 1H), 4.85 (d, 1H), 4.69 (dd, 1H), 4.49-4.40 (m, 1H), 4.12 (dd, 1H), 2.88 (s, 3H).

### (S)-N-(8,9-Difluoro-6-oxo-1,4,5,6-tetrahydro-2H-pyrano[3,4-c]isoquinolin-1-yl)-3-fluoro-N-methyl-4-(trifluoromethyl)benzamide (Compound 74)

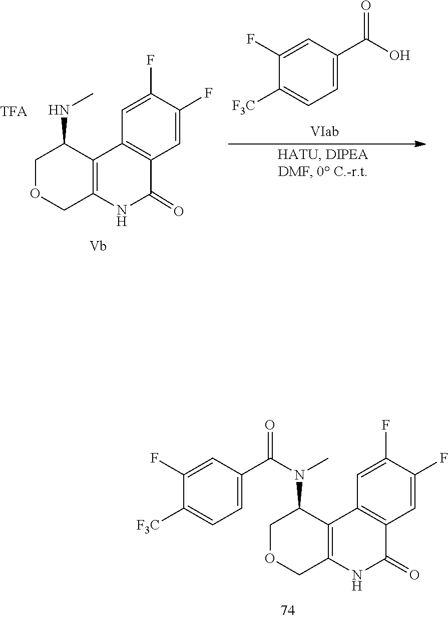

Enantiomerically pure (S)-N-(8,9-difluoro-6-oxo-1,4,5,6-tetrahydro-2H-pyrano[3,4-c]isoquinolin-1-yl)-3-fluoro-N-methyl-4-(trifluoromethyl)benzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-fluoro-4-(trifluoromethyl)benzoic acid (VIab). LCMS m/z found 457.2 [M+H]⁺; RT=4.69 min (Method A); ¹H NMR (400 MHz, Chloroform-d) δ 12.27 (s, 1H), 8.25 (dd, 1H), 7.69 (t, 1H), 7.48 (dd, 1H), 7.31-7.22 (m, 2H), 5.88-5.82 (m, 1H), 4.83 (d, 1H), 4.68 (dd, 1H), 4.42 (d, 1H), 4.10 (dd, 1H), 2.83 (s, 3H).

### (S)-4-Chloro-N-(8,9-difluoro-6-oxo-1,4,5,6-tetrahydro-2H-pyrano[3,4-c]isoquinolin-1-yl)-3-fluoro-N-methylbenzamide (Compound 75)

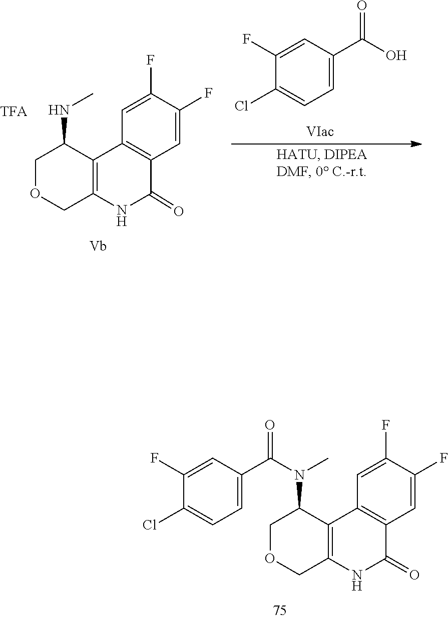

Enantiomerically pure (S)-4-chloro-N-(8,9-difluoro-6-oxo-1,4,5,6-tetrahydro-2H-pyrano[3,4-c]isoquinolin-1-yl)-3-fluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-chloro-3-fluorobenzoic acid (VIac). LCMS m/z found 423.1 [M+H]⁺; RT=4.61 min (Method A); ¹H NMR (400 MHz, Chloroform-d) δ 11.95 (s, 1H), 8.24 (dd, 1H), 7.53-7.43 (m, 2H), 7.29-7.19 (m, 1H), 7.15 (ddt, 1H), 5.83 (s, 1H), 4.80 (d, 1H), 4.71-4.61 (m, 1H), 4.40 (d, 1H), 4.09 (dd, 1H), 2.85 (s, 3H).

### (S)-N-(8,9-Difluoro-6-oxo-1,4,5,6-tetrahydro-2H-pyrano[3,4-c]isoquinolin-1-yl)-3,4,5-trifluoro-N-methylbenzamide (Compound 76)

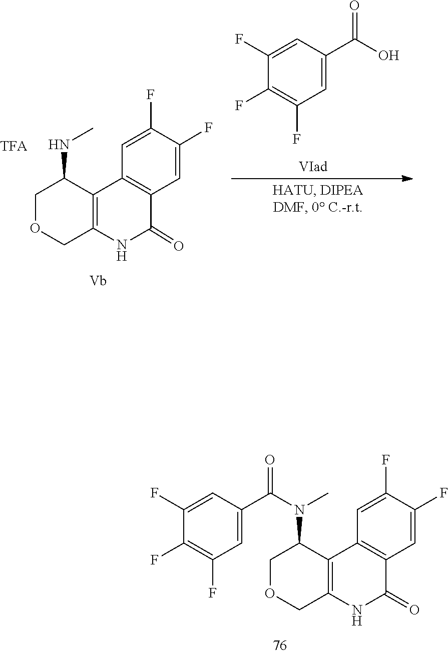

Enantiomerically pure (S)-N-(8,9-difluoro-6-oxo-1,4,5,6-tetrahydro-2H-pyrano[3,4-c]isoquinolin-1-yl)-3,4,5-trifluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3,4,5-trifluorobenzoic acid (VIad). LCMS m/z found 425.2 [M+H]⁺; RT=4.49 min (Method A); ¹H NMR (400 MHz, Chloroform-d) δ 12.05 (s, 1H), 8.24 (dd, 1H), 7.45 (dd, 1H), 7.08 (dd, 2H), 5.82-5.76 (m, 1H), 4.81 (d, 1H), 4.71-4.61 (m, 1H), 4.43-4.35 (m, 1H), 4.08 (dd, 1H), 2.86 (s, 3H).

### (S)-N-(8,9-Difluoro-6-oxo-1,4,5,6-tetrahydro-2H-pyrano[3,4-c]isoquinolin-1-yl)-5-fluoro-N-methylnicotinamide (Compound 83)

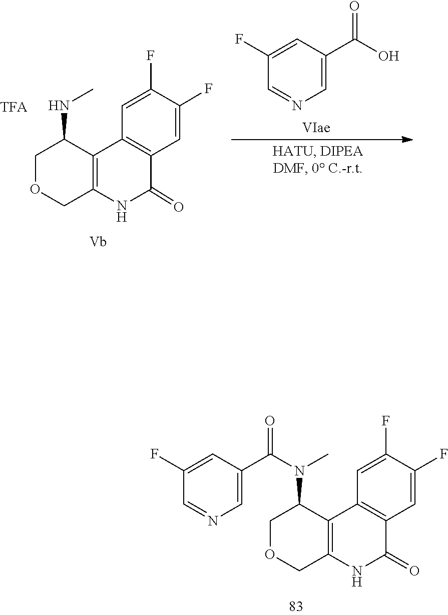 [SMILES: CN[C@@H]1COCc2[nH]c(=O)c3cc(F)c(F)cc3c21 ; O=C(O)c1cncc(F)c1 ; CN(C(=O)c1cncc(F)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-5-fluoro-N-methylnicotinamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5-fluoronicotinic acid (VIae). LCMS m/z found 390.1 [M+H]⁺; RT=3.27 min (Method A); ¹H NMR (400 MHz, Chloroform-d) δ 12.41 (s, 1H), 8.55 (d, 1H), 8.48 (t, 1H), 8.24 (dd, 1H), 7.56-7.43 (m, 2H), 5.87-5.82 (m, 1H), 4.84 (d, 1H), 4.73-4.63 (m, 1H), 4.47-4.38 (m, 1H), 4.10 (dd, 1H), 2.89 (s, 3H).

### (S)-5-Chloro-N-(8,9-difluoro-6-oxo-1,4,5,6-tetrahydro-2H-pyrano[3,4-c]isoquinolin-1-yl)-N-methylnicotinamide (Compound 84)

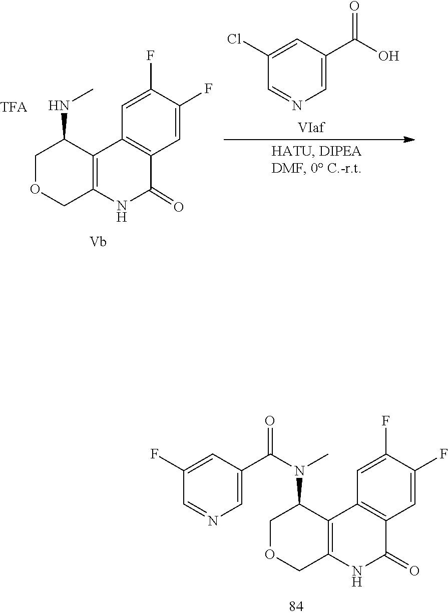 [SMILES: CN[C@@H]1COCc2[nH]c(=O)c3cc(F)c(F)cc3c21 ; O=C(O)c1cncc(Cl)c1 ; CN(C(=O)c1cncc(F)c1)[C@@H]1COCc2[nH]c(=O)c3cc(F)c(F)cc3c21]

Enantiomerically pure (S)-5-chloro-N-(8,9-difluoro-6-oxo-1,4,5,6-tetrahydro-2H-pyrano[3,4-c]isoquinolin-1-yl)-N-methylnicotinamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5-chloronicotinic acid (VIaf). LCMS m/z found 406.1/408.1 [M+H]⁺; RT=3.63 min (Method A); ¹H NMR (400 MHz, Chloroform-d) δ 12.12 (s, 1H), 8.68-8.62 (m, 1H), 8.54 (d, 1H), 8.25 (dd, 1H), 7.77 (dd, 1H), 7.48 (dd, 1H), 5.87-5.81 (m, 1H), 4.83 (d, 1H), 4.68 (dd, 1H), 4.42 (dd, 1H), 4.10 (dd, 1H), 2.89 (s, 3H).

### (S)-N-(8,9-Difluoro-6-oxo-1,4,5,6-tetrahydro-2H-pyrano[3,4-c]isoquinolin-1-yl)-2-fluoro-N-methylisonicotinamide (Compound 85)

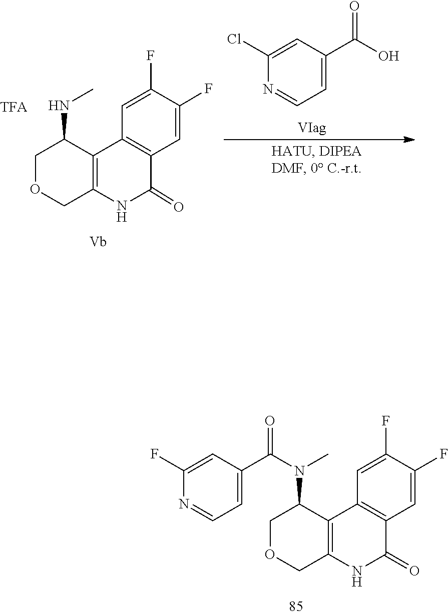 [SMILES: CN[C@@H]1COCc2[nH]c(=O)c3cc(F)c(F)cc3c21 ; O=C(O)c1ccnc(Cl)c1 ; CN(C(=O)c1ccnc(F)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-2-fluoro-N-methylisonicotinamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 2-fluoroisonicotinic acid (VIag). LCMS m/z found 390.2 [M+H]⁺; RT=3.36 min (Method A); ¹H NMR (400 MHz, Chloroform-d) δ 12.04 (s, 1H), 8.34 (dd, 1H), 8.25 (dd, 1H), 7.45 (dd, 1H), 7.18 (ddd, 1H), 6.94 (ddd, 1H), 5.87-5.81 (m, 1H), 4.82 (d, 1H), 4.67 (dd, 1H), 4.40 (d, 1H), 4.10 (dd, 1H), 2.82 (s, 3H).

### (S)-N-(8,9-Difluoro-6-oxo-1,4,5,6-tetrahydro-2H-pyrano[3,4-c]isoquinolin-1-yl)-3-(difluoromethyl)-N-methylbenzamide (Compound 86)

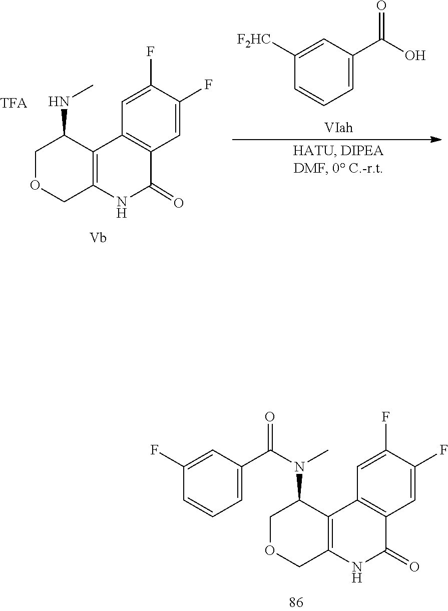 [SMILES: CN[C@@H]1COCc2[nH]c(=O)c3cc(F)c(F)cc3c21 ; O=C(O)c1cccc(C(F)F)c1 ; CN(C(=O)c1cccc(F)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-3-(difluoromethyl)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-(difluoromethyl)benzoic acid (VIah). LCMS m/z found 421.2 [M+H]⁺; RT=4.07 min (Method A); ¹H NMR (400 MHz, Chloroform-d) δ 12.38 (s, 1H), 8.23 (dd, 1H), 7.63-7.45 (m, 5H), 6.68 (t, 1H), 5.91-5.85 (m, 1H), 4.83 (d, 1H), 4.72-4.62 (m, 1H), 4.43 (dd, 1H), 4.10 (dd, 1H), 2.84 (s, 3H).

### (S)-N-((S)-8,9-Difluoro-6-oxo-1,4,5,6-tetrahydro-2H-pyrano[3,4-c]isoquinolin-1-yl)-2-hydroxy-N-methyl-3-phenylpropanamide (Compound 87)

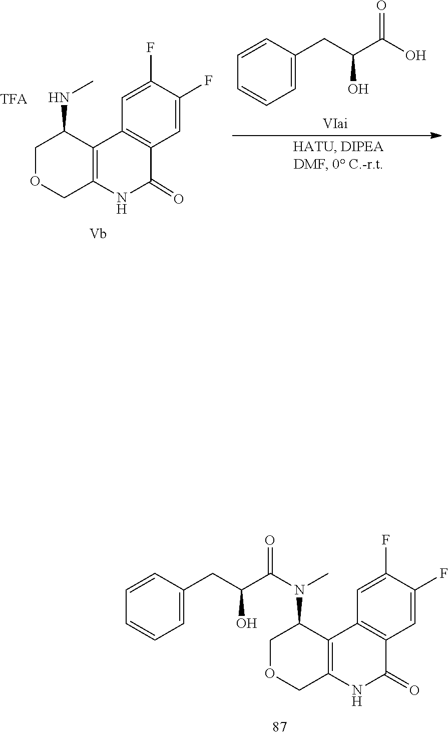

Enantiomerically pure (S)-N-((S)-8,9-difluoro-6-oxo-1,4,5,6-tetrahydro-2H-pyrano[3,4-c]isoquinolin-1-yl)-2-hydroxy-N-methyl-3-phenylpropanamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and (S)-2-hydroxy-3-phenylpropanoic acid (VIai). LCMS m/z found 415.2 [M+H]⁺; RT=3.69 min (Method A); ¹H NMR (400 MHz, Chloroform-d) δ 12.10 (s, 1H), 8.18 (dd, 1H), 7.39-7.27 (m, 3H), 7.32-7.25 (m, 3H), 5.75-5.69 (m, 1H), 4.77 (d, 1H), 4.66 (ddd, 1H), 4.64-4.49 (m, 1H), 4.06 (dd, 1H), 3.96-3.80 (m, 2H), 3.09 (dd, 1H), 3.01 (dd, 1H), 2.74 (s, 3H).

### (R)-N-((S)-8,9-Difluoro-6-oxo-1,4,5,6-tetrahydro-2H-pyrano[3,4-c]isoquinolin-1-yl)-2-hydroxy-N-methyl-3-phenylpropanamide (Compound 88)

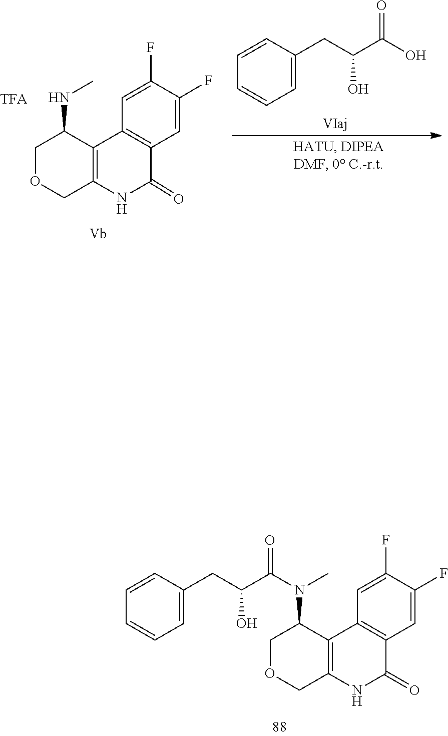

Enantiomerically pure (R)-N-((S)-8,9-difluoro-6-oxo-1,4,5,6-tetrahydro-2H-pyrano[3,4-c]isoquinolin-1-yl)-2-hydroxy-N-methyl-3-phenylpropanamide was synthesized in an analogous manner as described above from enantiomerically pure (S)-8,9-difluoro-1-(methylamino)-1,5-dihydro-2H-pyrano[3,4-c]isoquinolin-6(4H)-one, (Vb) and (R)-2-hydroxy-3-phenylpropanoic acid (VIaj). LCMS m/z found 415.2 [M+H]⁺; RT=3.95 min (Method A); ¹H NMR (400 MHz, Chloroform-d) δ 11.27 (bs, 1H), 8.23 (dd, 1H), 7.35-7.13 (m, 5H), 5.75-5.69 (m, 1H), 4.79 (d, 1H), 4.73-4.52 (m, 2H), 4.29 (dd, 1H), 4.00 (dd, 1H), 3.81 (d, 1H), 2.85 (s, 3H*), 2.85 (s, 1H, overlapped*), 2.84 (s, 1H).

### (S)-N-(8,9-Difluoro-6-oxo-1,4,5,6-tetrahydro-2H-pyrano[3,4-c]isoquinolin-1-yl)-N-methylbenzamide (Compound 91)

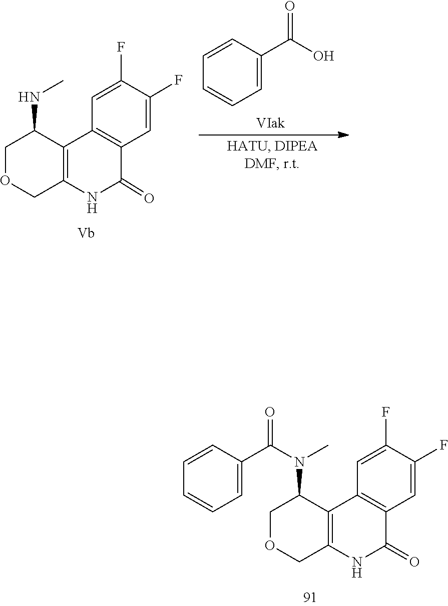

A stirred solution of benzoic acid (VIak, 20 mg, 0.17 mmol) in DMF (1 mL) was treated with diisopropylethylamine (105 μL, 0.60 mmol) and HATU (171 mg, 0.45 mmol) at room temperature for 10 min. Enantiomerically pure (S)-8,9-difluoro-1-(methylamino)-1,5-dihydro-2H-pyrano[3,4-c]isoquinolin-6(4H)-one (Vb, 40 mg of free base, 0.15 mmol) was then added and the reaction was continued for 2 h. The reaction was quenched by addition of 5 mL of saturated ammonium chloride (pH adjusted to ˜4 by the dropwise addition of 2 M HCl), and then extracted twice with ethyl acetate (30 mL each). The combined organic extracts were washed once with water (25 mL), and once with brine (15 mL), dried over sodium sulfate, filtered and the solvent evaporated. The product was isolated by flash chromatography (Silicagel, methanol/dichloromethane 0-2.5% gradient over 15 min), followed by trituration from minimum amount of methanol to afford enantiomerically pure (S)-N-(8,9-difluoro-6-oxo-1,4,5,6-tetrahydro-2H-pyrano[3,4-c]isoquinolin-1-yl)-N-methylbenzamide (123 mg, 23%). LCMS m/z found 371.2 [M+H]⁺; RT=4.24 min (Method A); ¹H NMR (400 MHz, Chloroform-d) δ 11.61 (s, 1H), 8.23 (dd, 1H), 7.56 (dd, 1H), 7.47-7.37 (m, 5H), 5.88 (s, 1H), 4.78 (d, 1H), 4.65 (dd, 1H), 4.43 (d, 1H), 4.10 (dd, 1H), 2.84 (s, 3H).

### (S)-N-(8,9-Difluoro-6-oxo-1,4,5,6-tetrahydro-2H-pyrano[3,4-c]isoquinolin-1-yl)-3,4-difluoro-N-methylbenzamide (Compound 92)

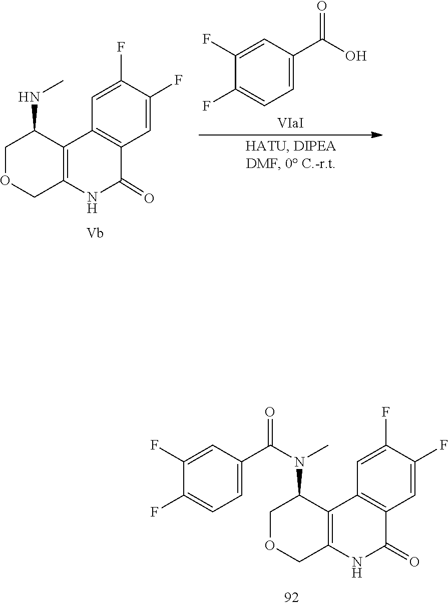

Enantiomerically pure (S)-N-(8,9-difluoro-6-oxo-1,4,5,6-tetrahydro-2H-pyrano[3,4-c]isoquinolin-1-yl)-3,4-difluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3,4-difluorobenzoic acid (VIal). LCMS m/z found 407.1 [M+H]⁺; RT=4.60 min (Method A); ¹H NMR (400 MHz, Chloroform-d) δ 11.80 (s, 1H), 8.24 (dd, 1H), 7.49 (dd, 1H), 7.33-7.13 (m, 3H), 5.82 (d, 1H), 4.80 (d, 1H), 4.70-4.61 (m, 1H), 4.41 (d, 1H), 4.09 (dd, 1H), 2.86 (s, 3H).

### (S)-N-((S)-8,9-Difluoro-6-oxo-1,4,5,6-tetrahydro-2H-pyrano[3,4-c]isoquinolin-1-yl)-2-hydroxy-N-methyl-2-phenylpropanamide (Compound 93)

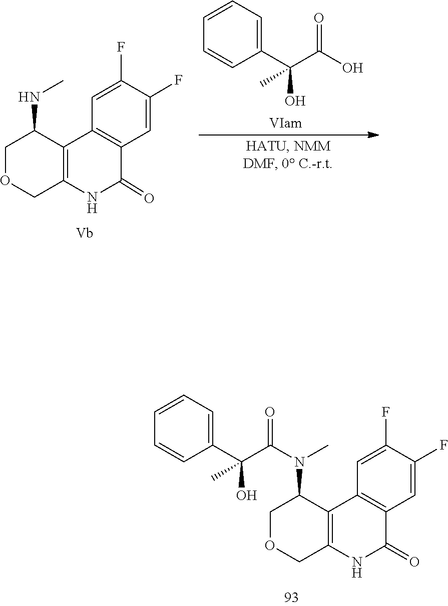

A stirred solution of (S)-2-hydroxy-2-phenylpropanoic acid (VIam, 28 mg, 0.17 mmol), enantiomerically pure (S)-8,9-difluoro-1-(methylamino)-1,5-dihydro-2H-pyrano[3,4-c]isoquinolin-6(4H)-one (Vb, 40 mg of free base, 0.15 mmol), and HATU (86 mg, 0.23 mmol) in DMF (0.5 mL) was treated with N-methylmorpholine (67 μL, 0.60 mmol) at 0° C., and the reaction was allowed to warm to room temperature and continued for 16 h. The reaction was quenched by addition of 5 mL of saturated ammonium chloride, diluted further with 5 mL of water and then extracted twice with ethyl acetate (30 mL each). The combined organic extracts were washed once with water (50 mL), and once with brine (20 mL), dried over sodium sulfate, filtered and the solvent evaporated. The product was isolated by flash chromatography (silica gel, ethyl acetate/hexanes 25-100% gradient) to afford enantiomerically pure (S)-N-((S)-8,9-difluoro-6-oxo-1,4,5,6-tetrahydro-2H-pyrano[3,4-c]isoquinolin-1-yl)-2-hydroxy-N-methyl-2-phenylpropanamide (24 mg, 39%). LCMS m/z found 415.2 [M+H]⁺; RT=4.51 min (Method A); ¹H NMR (400 MHz, Chloroform-d) δ 11.92 (s, 1H), 8.16 (dd, 1H), 7.45-7.20 (m, 6H), 5.82-5.76 (m, 1H), 4.85 (s, 1H), 4.66 (d, 1H), 4.60-4.50 (m, 1H), 4.23 (d, 1H), 4.01 (dd, 1H), 2.48 (s, 3H), 1.81 (s, 3H).

### (R)-N-((S)-8,9-Difluoro-6-oxo-1,4,5,6-tetrahydro-2H-pyrano[3,4-c]isoquinolin-1-yl)-2-hydroxy-N-methyl-2-phenylpropanamide (Compound 94)

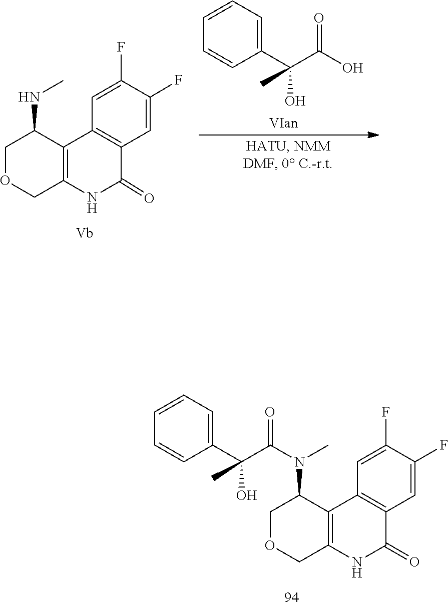

Enantiomerically pure (R)-N-((S)-8,9-difluoro-6-oxo-1,4,5,6-tetrahydro-2H-pyrano[3,4-c]isoquinolin-1-yl)-2-hydroxy-N-methyl-2-phenylpropanamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and (R)-2-hydroxy-2-phenylpropanoic acid (VIan). LCMS m/z found 415.3 [M+H]⁺; RT=4.39 min (Method A); ¹H NMR (400 MHz, Chloroform-d/methanol-d₄) δ 8.05 (dd, 1H), 7.38-7.30 (m, 2H), 7.30-7.12 (m, 4H), 5.63 (d, 1H), 4.46 (d, 1H), 4.42-4.33 (m, 1H), 3.90 (dd, 1H), 3.33 (d, 1H), 3.21 (s, exch. Hs), 2.42 (s, 3H), 1.81 (s, 3H).

### (S)-N-((S)-8,9-Difluoro-6-oxo-1,4,5,6-tetrahydro-2H-pyrano[3,4-c]isoquinolin-1-yl)-2-hydroxy-N-methyl-2-phenylacetamide (Compound 95)

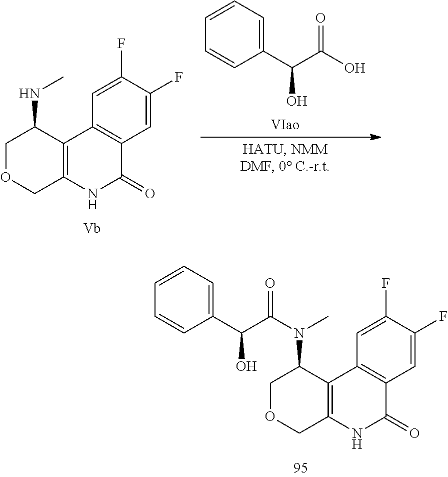

Enantiomerically pure (S)-N-((S)-8,9-difluoro-6-oxo-1,4,5,6-tetrahydro-2H-pyrano[3,4-c]isoquinolin-1-yl)-2-hydroxy-N-methyl-2-phenylacetamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and (S)-2-hydroxy-2-phenylacetic acid (VIao). LCMS m/z found 401.2 [M+H]⁺; RT=4.06 min (Method A); ¹H NMR (400 MHz, Chloroform-d) δ 12.27 (s, 1H), 8.18 (dd, 1H), 7.53-7.31 (m, 6H), 5.82 (m, 1H), 5.25 (d, 1H), 4.83 (d, 1H), 4.69 (d, 1H), 4.56 (dd, 1H), 4.04 (dd, 1H), 3.94 (dd, 1H), 2.62 (s, 3H).

### (R)-N-((S)-8,9-Difluoro-6-oxo-1,4,5,6-tetrahydro-2H-pyrano[3,4-c]isoquinolin-1-yl)-2-hydroxy-N-methyl-2-phenylacetamide (Compound 96)

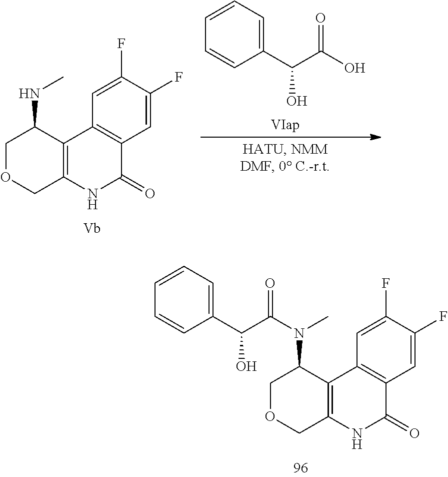

Enantiomerically pure (R)-N-((S)-8,9-difluoro-6-oxo-1,4,5,6-tetrahydro-2H-pyrano[3,4-c]isoquinolin-1-yl)-2-hydroxy-N-methyl-2-phenylacetamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and (R)-2-hydroxy-2-phenylacetic acid (VIap). LCMS m/z found 401.1 [M+H]⁺; RT=4.00 min (Method A); ¹H NMR (400 MHz, Chloroform-d) δ 12.08 (s, 1H), 8.09 (dd, 1H), 7.39-7.29 (m, 5H), 7.05 (dd, 1H), 5.71-5.65 (m, 1H), 5.26 (d, 1H), 4.82 (d, 1H), 4.71 (d, 1H), 4.61-4.47 (m, 1H), 4.34-4.26 (m, 1H), 3.98 (dd, 1H), 2.65 (s, 3H).

### (R)-2-(3-Chlorophenyl)-N-((S)-8,9-difluoro-6-oxo-1,4,5,6-tetrahydro-2H-pyrano[3,4-c]isoquinolin-1-yl)-2-hydroxy-N-methylacetamide (Compound 138)

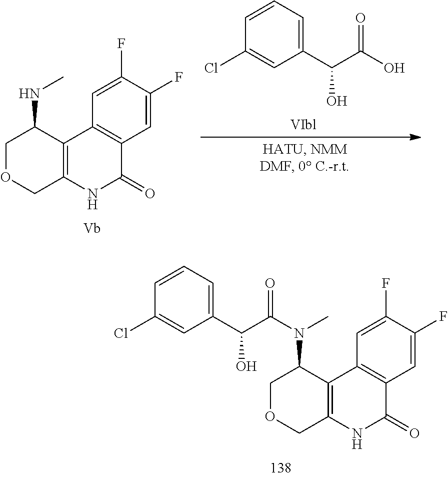

Enantiomerically (R)-2-(3-chlorophenyl)-N-((S)-8,9-difluoro-6-oxo-1,4,5,6-tetrahydro-2H-pyrano[3,4-c]isoquinolin-1-yl)-2-hydroxy-N-methylacetamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and (R)-2-(3-chlorophenyl)-2-hydroxyacetic acid (VIbI). LCMS m/z 435.1/437.2 [M+H]⁺; RT=5.94 min (Method A); ¹H NMR (400 MHz, Chloroform-d) δ 11.64 (s, 1H), 8.04 (dd, 1H), 7.42 (q, 1H), 7.41-7.28 (m, 3H), 6.97 (dd, 1H), 5.84 (d, 1H), 5.53-5.46 (m, 2H), 4.55 (d, 1H), 4.40 (dd, 1H), 4.05 (dd, 1H), 3.95 (dd, 1H), 2.73 (s, 3H).

### (S)-N-(8,9-Difluoro-6-oxo-1,4,5,6-tetrahydro-2H-pyrano[3,4-c]isoquinolin-1-yl)-3,5-difluoro-N-methyl-4-(trifluoromethyl)benzamide (Compound 139)

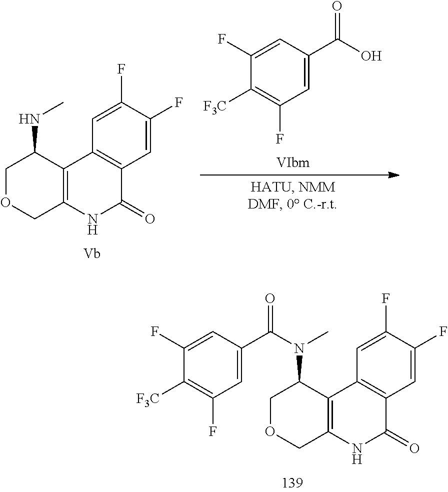

Enantiomerically pure (S)-N-(8,9-difluoro-6-oxo-1,4,5,6-tetrahydro-2H-pyrano[3,4-c]isoquinolin-1-yl)-3,5-difluoro-N-methyl-4-(trifluoromethyl)benzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3,5-difluoro-4-(trifluoromethyl) benzoic acid (VIbm). LCMS m/z found 411.1/413.1 [M+H]⁺; RT=5.86 min (Method A); ¹H NMR (400 MHz, DMSO-d₆) δ 11.70 (s, 1H), 8.12 (dd, 1H), 7.78 (d, 1H), 7.43-7.29 (m, 2H), 5.60 (d, 1H), 4.59 (d, 1H), 4.50-4.41 (m, 1H), 4.18 (d, 1H), 4.00 (dd, 1H), 2.85 (s, 3H).

### (S)-5-Chloro-N-(8,9-difluoro-6-oxo-1,4,5,6-tetrahydro-2H-pyrano[3,4-c]isoquinolin-1-yl)-N-methylthiophene-3-carboxamide (Compound 140)

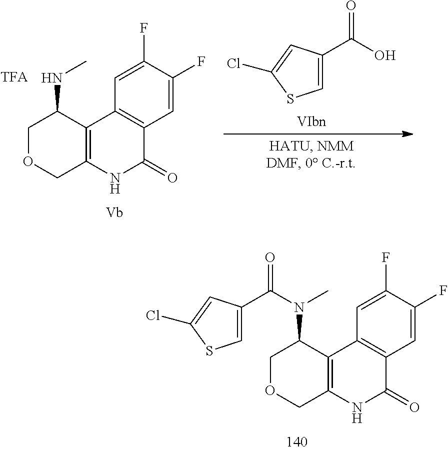

Enantiomerically pure (S)-5-chloro-N-(8,9-difluoro-6-oxo-1,4,5,6-tetrahydro-2H-pyrano[3,4-c]isoquinolin-1-yl)-N-methylthiophene-3-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5-chlorothiophene-3-carboxylic acid (VIbn). LCMS m/z found 411.1/413.1 [M+H]⁺; RT=5.86 min (Method A); ¹H NMR (400 MHz, DMSO-d₆) δ 11.70 (s, 1H), 8.12 (dd, 1H), 7.78 (d, 1H), 7.43-7.29 (m, 2H), 5.60 (d, 1H), 4.59 (d, 1H), 4.50-4.41 (m, 1H), 4.18 (d, 1H), 4.00 (dd, 1H), 2.85 (s, 3H).

### (S)-2-Chloro-N-(8,9-difluoro-6-oxo-1,4,5,6-tetrahydro-2H-pyrano[3,4-c]isoquinolin-1-yl)-N-methylthiazole-5-carboxamide (Compound 141)

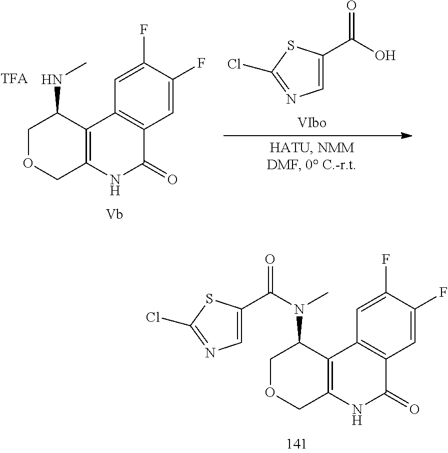

Enantiomerically pure (S)-2-chloro-N-(8,9-difluoro-6-oxo-1,4,5,6-tetrahydro-2H-pyrano[3,4-c]isoquinolin-1-yl)-N-methylthiazole-5-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 2-chlorothiazole-5-carboxylic acid (VIbo). LCMS m/z found 412.0/414.1 [M+H]⁺; RT=5.83 min (Method A); ¹H NMR (400 MHz, DMSO-d₆) δ 11.73 (s, 1H), 8.17-8.07 (m, 2H), 7.32 (dd, 1H), 5.58 (s, 1H), 4.62 (d, 1H), 4.46 (d, 1H), 4.18 (d, 1H), 3.98 (dd, 1H), 3.07 (s, 3H).

### (2R,3R)-2-Amino-N-((S)-8,9-difluoro-6-oxo-1,4,5,6-tetrahydro-2H-pyrano[3,4-c]isoquinolin-1-yl)-3-hydroxy-N-methylbutanamide, formic acid salt (Compound 142)

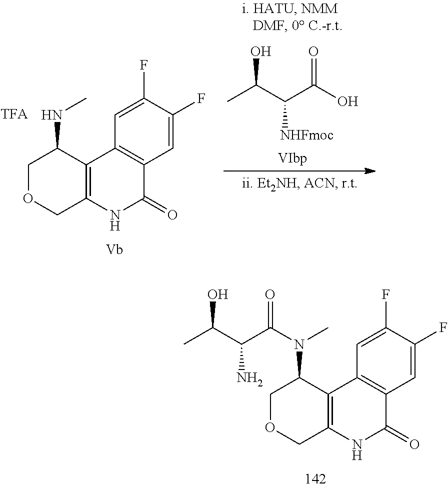

Enantiomerically pure (2R,3R)-2-amino-N-((S)-8,9-difluoro-6-oxo-1,4,5,6-tetrahydro-2H-pyrano[3,4-c]isoquinolin-1-yl)-3-hydroxy-N-methylbutanamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and (((9H-fluoren-9-yl)methoxy)carbonyl)-D-allothreonine (VIbp), followed by deprotection of the intermediate Fmoc-protected amine with diethylamine (10 eq.) in acetonitrile for 1 h at room temperature. The product was purified by reverse phase preparative hplc (C18 Column, water/acetonitrile 5-70% gradient, modified with 0.05% formic acid) and isolated as the formic acid salt. LCMS m/z found 368.2 [M+H]⁺; RT=4.15 min (Method A); ¹H NMR (400 MHz, DMSO-d₆) δ 8.26 (s, 1H), 8.08 (dd, 1H), 7.31 (dd, 1H), 5.52-5.46 (m, 1H), 4.57 (d, 1H), 4.42 (dd, 1H), 4.02-3.88 (m, 2H), 3.66-3.57 (m, 2H), 2.85 (s, 3H), 1.10 (d, 3H).

### (S)-N-(8,9-Difluoro-6-oxo-1,4,5,6-tetrahydro-2H-pyrano[3,4-c]isoquinolin-1-yl)-4,5,6-trifluoro-N-methyl-1H-indole-2-carboxamide (Compound 97)

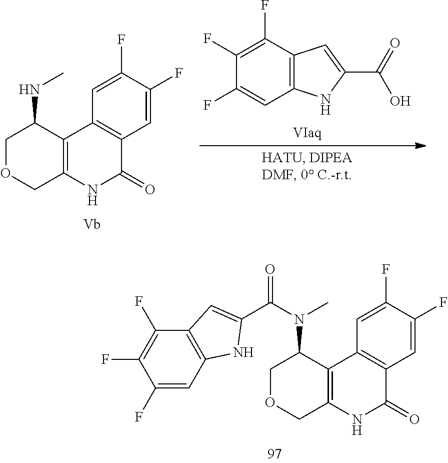

Enantiomerically pure (S)-N-(8,9-difluoro-6-oxo-1,4,5,6-tetrahydro-2H-pyrano[3,4-c]isoquinolin-1-yl)-4,5,6-trifluoro-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4,5,6-trifluoro-1H-indole-2-carboxylic acid (VIaq). LCMS m/z found 464.2 [M+H]⁺; RT=5.60 min (Method A); ¹H NMR (400 MHz, DMSO-d₆) δ 12.24 (s, 1H), 11.74 (s, 1H), 8.12 (dd, 1H), 7.42 (dd, 1H), 7.27 (dd, 1H), 7.14-7.08 (m, 1H), 5.74 (s, 1H), 4.64 (d, 1H), 4.53-4.43 (m, 1H), 4.17 (d, 1H), 4.04 (dd, 1H), 3.16 (s, 3H).

### (S)-N-(8,9-Difluoro-6-oxo-1,4,5,6-tetrahydro-2H-pyrano[3,4-c]isoquinolin-1-yl)-N-methyl-4-(trifluoromethyl)benzamide (Compound 98)

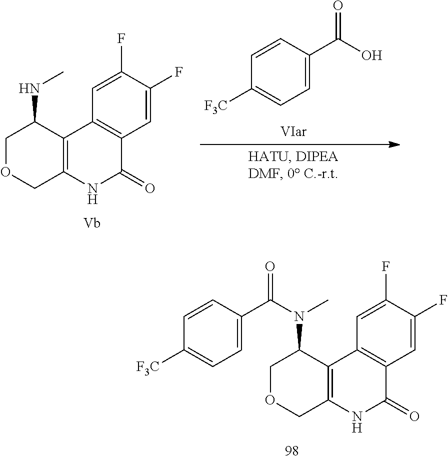 [SMILES: CN[C@@H]1COCc2[nH]c(=O)c3cc(F)c(F)cc3c21 ; O=C(O)c1ccc(C(F)(F)F)cc1 ; CN(C(=O)c1ccc(C(F)(F)F)cc1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-N-methyl-4-(trifluoromethyl)benzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-(trifluoromethyl)benzoic acid (VIar). LCMS m/z found 439.2 [M+H]⁺; RT=5.27 min (Method A); ¹H NMR (400 MHz, DMSO-d₆) δ 11.72 (s, 1H), 8.14 (dd, 1H), 7.84 (d, 2H), 7.66-7.59 (m, 2H), 7.49 (dd, 1H), 5.69 (d, 1H), 4.59 (d, 1H), 4.47 (dd, 1H), 4.31 (d, 1H), 4.08-3.99 (m, 1H), 2.68 (s, 3H).

### (S)-4-Chloro-N-(8,9-difluoro-6-oxo-1,4,5,6-tetrahydro-2H-pyrano[3,4-c]isoquinolin-1-yl)-N-methylbenzamide (Compound 99)

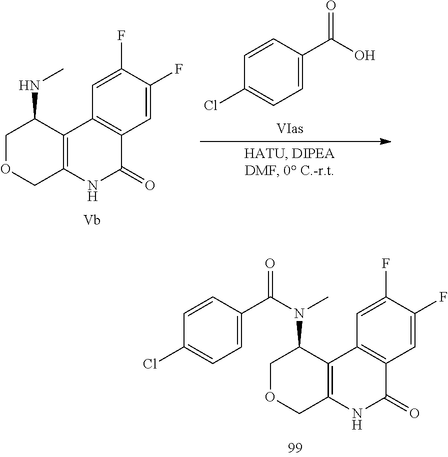 [SMILES: CN[C@@H]1COCc2[nH]c(=O)c3cc(F)c(F)cc3c21 ; O=C(O)c1ccc(Cl)cc1 ; CN(C(=O)c1ccc(Cl)cc1)[C@@H]1COCc2[nH]c(=O)c3cc(F)c(F)cc3c21]

Enantiomerically pure (S)-4-chloro-N-(8,9-difluoro-6-oxo-1,4,5,6-tetrahydro-2H-pyrano[3,4-c]isoquinolin-1-yl)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-chlorobenzoic acid (VIas). LCMS m/z found 405.2/407.2 [M+H]⁺; RT=5.10 min (Method A); ¹H NMR (400 MHz, DMSO-d₆) δ 11.71 (s, 1H), 8.13 (dd, 1H), 7.57-7.40 (m, 5H), 5.66 (s, 1H), 4.58 (d, 1H), 4.46 (d, 1H), 4.27 (d, 1H), 4.03 (dd, 1H), 2.70 (s, 3H).

### (S)-4-Cyano-N-(8,9-difluoro-6-oxo-1,4,5,6-tetrahydro-2H-pyrano[3,4-c]isoquinolin-1-yl)-3-fluoro-N-methylbenzamide (Compound 100)

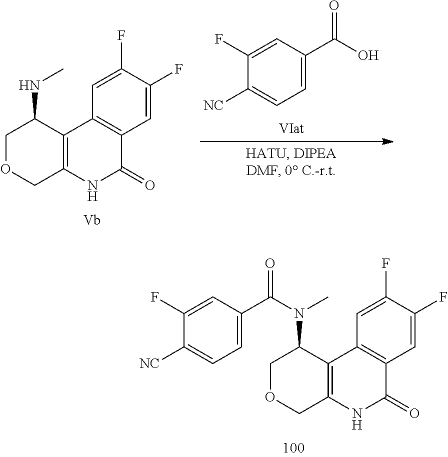 [SMILES: CN[C@@H]1COCc2[nH]c(=O)c3cc(F)c(F)cc3c21 ; N#Cc1ccc(C(=O)O)cc1F ; CN(C(=O)c1ccc(C#N)c(F)c1)[C@@H]1COCc2[nH]c(=O)c3cc(F)c(F)cc3c21]

Enantiomerically pure (S)-4-cyano-N-(8,9-difluoro-6-oxo-1,4,5,6-tetrahydro-2H-pyrano[3,4-c]isoquinolin-1-yl)-3-fluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-cyano-3-fluorobenzoic acid (VIat). LCMS m/z found 414.2 [M+H]⁺; RT=4.65 min (Method A); ¹H NMR (400 MHz, DMSO-d₆) δ 11.71 (s, 1H), 8.18-8.08 (m, 1H), 8.04 (ddd, 1H), 7.68 (dd, 1H), 7.50-7.37 (m, 2H), 5.65 (s, 1H), 4.59 (d, 1H), 4.46 (d, 1H), 4.32 (d, 1H), 4.01 (dd, 1H), 2.68 (s, 3H).

### (S)-N-(8,9-Difluoro-6-oxo-1,4,5,6-tetrahydro-2H-pyrano[3,4-c]isoquinolin-1-yl)-3-(difluoromethyl)-4-fluoro-N-methylbenzamide (Compound 109)

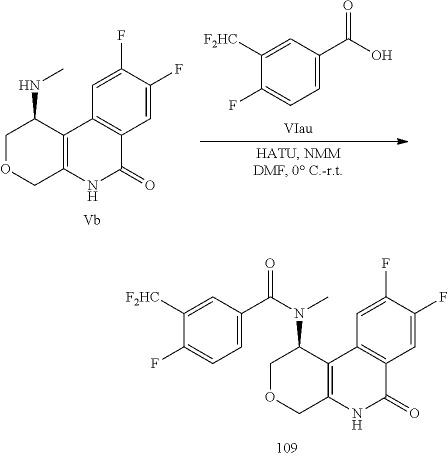 [SMILES: CN[C@@H]1COCc2[nH]c(=O)c3cc(F)c(F)cc3c21 ; O=C(O)c1ccc(F)c(C(F)F)c1 ; CN(C(=O)c1ccc(F)c(C(F)F)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-3-(difluoromethyl)-4-fluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-(difluoromethyl)-4-fluorobenzoic acid (VIau). LCMS m/z found 439.2 [M+H]⁺; RT=4.96 min (Method A); ¹H NMR (400 MHz, DMSO-d₆) δ 11.70 (s, 1H), 8.13 (dd, 1H), 7.76-7.63 (m, 2H), 7.53-7.42 (m, 2H), 7.24 (t, 1H), 5.67 (d, 1H), 4.59 (d, 1H), 4.46 (d, 1H), 4.31 (d, 1H), 4.02 (dd, 1H), 2.72 (s, 3H).

### (S)-N-(8,9-Difluoro-6-oxo-1,4,5,6-tetrahydro-2H-pyrano[3,4-c]isoquinolin-1-yl)-4-(difluoromethyl)-3-fluoro-N-methylbenzamide (Compound 110)

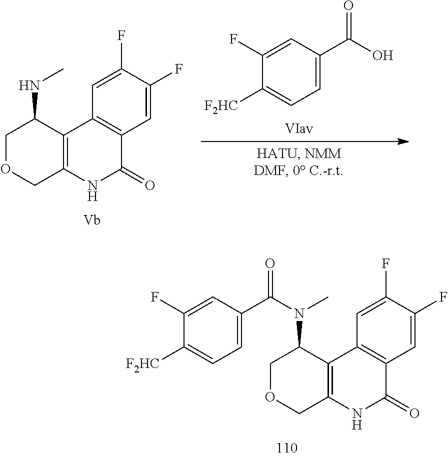 [SMILES: CN[C@@H]1COCc2[nH]c(=O)c3cc(F)c(F)cc3c21 ; O=C(O)c1ccc(C(F)F)c(F)c1 ; CN(C(=O)c1ccc(C(F)F)c(F)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-4-(difluoromethyl)-3-fluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-(difluoromethyl)-3-fluorobenzoic acid (VIav). LCMS m/z found 439.2 [M+H]⁺; RT=5.02 min (Method A); ¹H NMR (400 MHz, DMSO-d₆) δ 11.71 (s, 1H), 8.13 (dd, 1H), 7.73 (t, 1H), 7.54-7.42 (m, 2H), 7.37-7.33 (m, 1H), 7.18 (d, 1H), 5.66 (t, 1H), 4.59 (d, 1H), 4.46 (d, 1H), 4.30 (d, 1H), 4.02 (dd, 1H), 2.69 (s, 3H).

### (S)-N-(8,9-Difluoro-6-oxo-1,4,5,6-tetrahydro-2H-pyrano[3,4-c]isoquinolin-1-yl)-4-(difluoromethyl)-N-methylbenzamide (Compound 111)

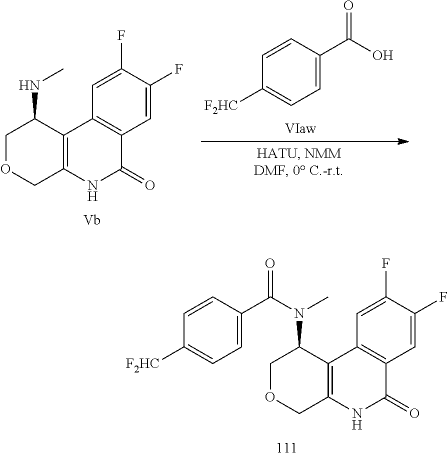 [SMILES: CN[C@@H]1COCc2[nH]c(=O)c3cc(F)c(F)cc3c21 ; O=C(O)c1ccc(C(F)F)cc1 ; CN(C(=O)c1ccc(C(F)F)cc1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-4-(difluoromethyl)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-(difluoromethyl)benzoic acid (VIaw). LCMS m/z found 421.1 [M+H]⁺; RT=4.98 min (Method A); ¹H NMR (400 MHz, DMSO-d₆) δ 11.71 (s, 1H), 8.13 (dd, 1H), 7.66 (dt, 2H), 7.57-7.44 (m, 2H), 7.08 (t, 1H), 5.72-5.66 (m, 1H), 4.59 (d, 1H), 4.51-4.42 (m, 1H), 4.28 (d, 1H), 4.09-3.97 (m, 1H), 2.69 (d, 3H).

### (S)-3-Chloro-N-(8,9-difluoro-6-oxo-1,4,5,6-tetrahydro-2H-pyrano[3,4-c]isoquinolin-1-yl)-4-fluoro-N-methylbenzamide (Compound 112)

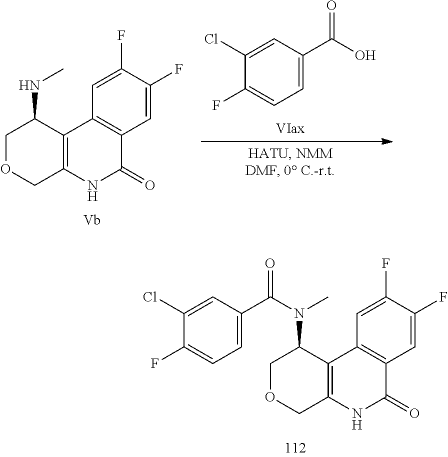 [SMILES: CN[C@@H]1COCc2[nH]c(=O)c3cc(F)c(F)cc3c21 ; O=C(O)c1ccc(F)c(Cl)c1 ; CN(C(=O)c1ccc(F)c(Cl)c1)[C@@H]1COCc2[nH]c(=O)c3cc(F)c(F)cc3c21]

Enantiomerically pure (S)-3-chloro-N-(8,9-difluoro-6-oxo-1,4,5,6-tetrahydro-2H-pyrano[3,4-c]isoquinolin-1-yl)-4-fluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-chloro-4-fluorobenzoic acid (VIax). LCMS m/z found 423.1/425.2 [M+H]⁺; RT=5.30 min (Method A); ¹H NMR (400 MHz, DMSO-d₆) δ 11.70 (s, 1H), 8.12 (dd, 1H), 7.72 (dd, 1H), 7.55-7.38 (m, 3H), 5.65 (d, 1H), 4.59 (d, 1H), 4.46 (d, 1H), 4.30 (d, 1H), 4.01 (dd, 1H), 2.71 (s, 3H).

### (S)-N-(8,9-Difluoro-6-oxo-1,4,5,6-tetrahydro-2H-pyrano[3,4-c]isoquinolin-1-yl)-N-methyl-3-(trifluoromethyl)-1H-pyrazole-5-carboxamide (Compound 125)

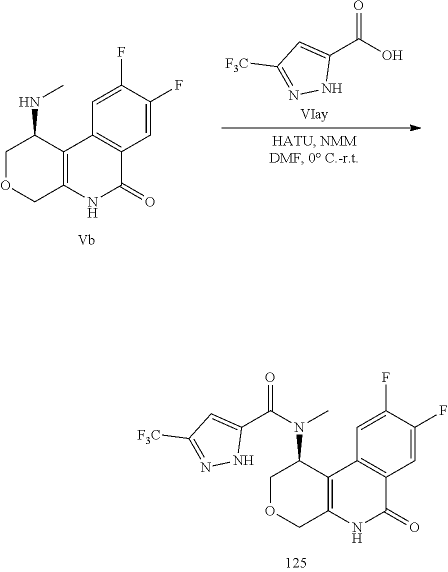 [SMILES: CN[C@@H]1COCc2[nH]c(=O)c3cc(F)c(F)cc3c21 ; O=C(O)c1cc(C(F)(F)F)n[nH]1 ; CN(C(=O)c1cc(C(F)(F)F)n[nH]1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-N-methyl-3-(trifluoromethyl)-1H-pyrazole-5-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-(trifluoromethyl)-1H-pyrazole-5-carboxylic acid (VIay). LCMS m/z found 429.2 [M+H]⁺; RT=4.80 min (Method A); ¹H NMR (400 MHz, DMSO-d₆) δ 11.74 (s, 1H), 8.12 (dd, 1H), 7.36 (dd, 1H), 7.21 (d, 1H), 5.67 (d, 1H), 4.63 (d, 1H), 4.47 (d, 1H), 4.16 (d, 1H), 4.01 (dd, 1H), 3.02 (s, 3H).

### (S)-N-(8,9-Difluoro-6-oxo-1,4,5,6-tetrahydro-2H-pyrano[3,4-c]isoquinolin-1-yl)-3-(4-fluorophenyl)-N-methyl-1H-pyrazole-5-carboxamide (Compound 126)

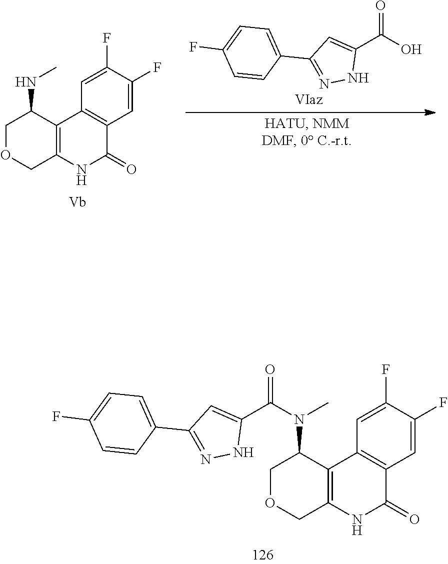

Enantiomerically pure (S)-N-(8,9-difluoro-6-oxo-1,4,5,6-tetrahydro-2H-pyrano[3,4-c]isoquinolin-1-yl)-3-(4-fluorophenyl)-N-methyl-1H-pyrazole-5-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-(4-fluorophenyl)-1H-pyrazole-5-carboxylic acid (VIaz). LCMS m/z found 455.2 [M+H]⁺; RT=5.01 min (Method A); ¹H NMR (400 MHz, DMSO-d₆) δ 13.69 (br s, 1H), 11.73 (s, 1H), 8.13 (dd, 1H), 7.92-7.84 (m, 2H), 7.44-7.27 (m, 3H), 7.17 (d, 1H), 5.71 (s, 1H), 4.62 (d, 1H), 4.48 (d, 1H), 4.15 (d, 1H), 4.03 (d, 1H), 3.11 (s, 3H).

### (S)-N-(8,9-Difluoro-6-oxo-1,4,5,6-tetrahydro-2H-pyrano[3,4-c]isoquinolin-1-yl)-3-fluoro-N-methylbenzamide (Compound 127)

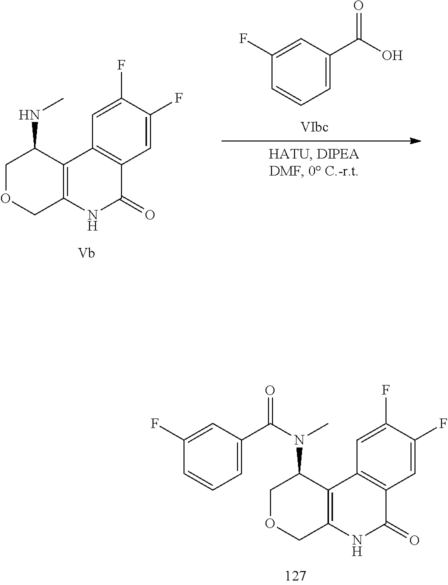 [SMILES: CN[C@@H]1COCc2[nH]c(=O)c3cc(F)c(F)cc3c21 ; O=C(O)c1cccc(F)c1 ; CN(C(=O)c1cccc(F)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-3-fluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-fluorobenzoic acid (VIbc), using DIPEA instead of NMM as a base. LCMS m/z found 389.2 [M+H]⁺; RT=5.19 min (Method A); ¹H NMR (400 MHz, DMSO-d₆) δ 11.69 (s, 1H), 8.13 (t, 1H), 7.54-7.45 (m, 2H), 7.34-7.28 (m, 2H), 7.21 (d, 1H), 5.67 (s, 1H), 4.58 (d, 1H), 4.46 (d, 1H), 4.27 (d, 1H), 4.04-4.01 (m, 1H), 2.7 (s, 3H).

### (S)-3-Chloro-N-(8,9-difluoro-6-oxo-1,4,5,6-tetrahydro-2H-pyrano[3,4-c]isoquinolin-1-yl)-N-methylbenzamide (Compound 128)

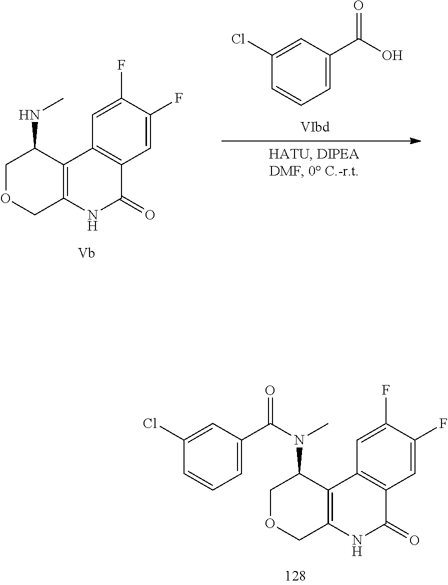 [SMILES: CN[C@@H]1COCc2[nH]c(=O)c3cc(F)c(F)cc3c21 ; O=C(O)c1cccc(Cl)c1 ; CN(C(=O)c1cccc(Cl)c1)[C@@H]1COCc2[nH]c(=O)c3cc(F)c(F)cc3c21]

Enantiomerically pure (S)-3-chloro-N-(8,9-difluoro-6-oxo-1,4,5,6-tetrahydro-2H-pyrano[3,4-c]isoquinolin-1-yl)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-chlorobenzoic acid (VIbd). LCMS m/z found 405.2/407.2 [M+H]⁺; RT=5.47 min (Method A); ¹H NMR (400 MHz, DMSO-d₆) δ 11.69 (s, 1H), 8.13 (t, 1H), 7.54-7.45 (m, 4H), 7.34 (d, 1H), 5.67 (s, 1H), 4.58 (d, 1H), 4.46 (d, 1H), 4.29 (d, 1H), 4.02 (d, 1H), 2.69 (s, 3H).

### (S)-3-Bromo-N-(8,9-difluoro-6-oxo-1,4,5,6-tetrahydro-2H-pyrano[3,4-c]isoquinolin-1-yl)-N-methylbenzamide (Compound 129)

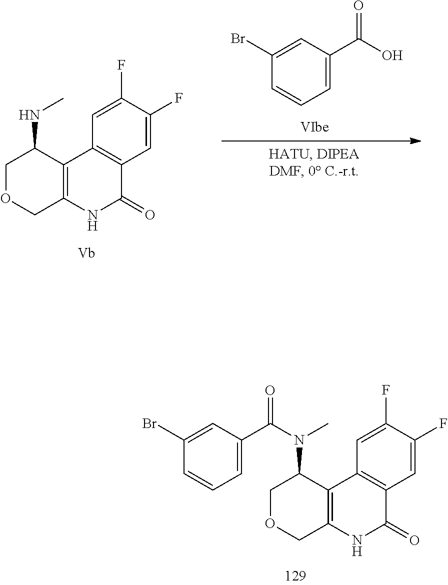 [SMILES: CN[C@@H]1COCc2[nH]c(=O)c3cc(F)c(F)cc3c21 ; O=C(O)c1cccc(Br)c1 ; CN(C(=O)c1cccc(Br)c1)[C@@H]1COCc2[nH]c(=O)c3cc(F)c(F)cc3c21]

Enantiomerically pure (S)-3-bromo-N-(8,9-difluoro-6-oxo-1,4,5,6-tetrahydro-2H-pyrano[3,4-c]isoquinolin-1-yl)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-bromobenzoic acid (VIbe). LCMS m/z found 449.2/451.1 [M+H]⁺; RT=5.70 min (Method A); ¹H NMR (400 MHz, DMSO-d₆) δ 11.69 (s, 1H), 8.15-8.11 (m, 1H), 7.67-7.63 (m, 2H), 7.5-7.37 (m, 3H), 5.66 (s, 1H), 4.58 (d, 1H), 4.46 (d, 1H), 4.29 (d, 1H), 4.03-3.99 (m, 1H), 2.69 (s, 3H).

### (S)-3-Bromo-N-(8,9-difluoro-6-oxo-1,4,5,6-tetrahydro-2H-pyrano[3,4-c]isoquinolin-1-yl)-4-fluoro-N-methylbenzamide (Compound 130)

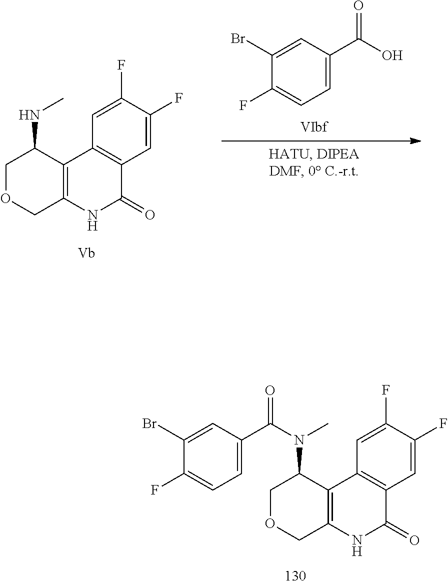 [SMILES: CN[C@@H]1COCc2[nH]c(=O)c3cc(F)c(F)cc3c21 ; O=C(O)c1ccc(F)c(Br)c1 ; CN(C(=O)c1ccc(F)c(Br)c1)[C@@H]1COCc2[nH]c(=O)c3cc(F)c(F)cc3c21]

Enantiomerically pure (S)-3-bromo-N-(8,9-difluoro-6-oxo-1,4,5,6-tetrahydro-2H-pyrano[3,4-c]isoquinolin-1-yl)-4-fluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-bromo-3-fluorobenzoic acid (VIbf). LCMS m/z found 467.2/469.2 [M+H]⁺; RT=5.83 min (Method A); ¹H NMR (400 MHz, DMSO-d₆) δ 11.69 (s, 1H), 8.13 (t, 1H), 7.80 (t, 1H), 7.51-7.44 (m, 2H), 7.18 (d, 1H), 5.65 (s, 1H), 4.59 (d, 1H), 4.46 (d, 1H), 4.29 (d, 1H), 4.03-4.0 (m, 1H), 2.7 (s, 3H).

### (S)-4-Bromo-N-(8,9-difluoro-6-oxo-1,4,5,6-tetrahydro-2H-pyrano[3,4-c]isoquinolin-1-yl)-N-methylbenzamide (Compound 131)

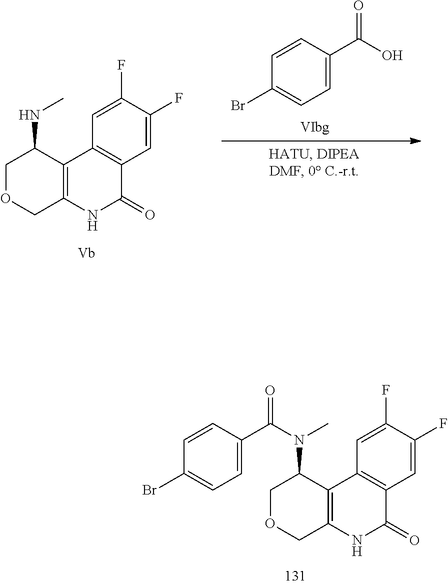 [SMILES: CN[C@@H]1COCc2[nH]c(=O)c3cc(F)c(F)cc3c21 ; O=C(O)c1ccc(Br)cc1 ; CN(C(=O)c1ccc(Br)cc1)[C@@H]1COCc2[nH]c(=O)c3cc(F)c(F)cc3c21]

Enantiomerically pure (S)-4-bromo-N-(8,9-difluoro-6-oxo-1,4,5,6-tetrahydro-2H-pyrano[3,4-c]isoquinolin-1-yl)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-bromo-benzoic acid (VIbg). LCMS m/z found 449.2/451.1 [M+H]⁺; RT=5.71 min (Method A); ¹H NMR (400 MHz, DMSO-d₆) δ 11.69 (s, 1H), 8.15-8.11 (m, 1H), 7.66 (d, 2H), 7.49-7.44 (m, 1H), 7.36 (d, 2H), 5.66 (s, 1H), 4.58 (d, 1H), 4.46 (d, 1H), 4.26 (d, 1H), 4.04-4.0 (m, 1H), 2.69 (s, 3H).

### (S)-3-Cyano-N-(8,9-difluoro-6-oxo-1,4,5,6-tetrahydro-2H-pyrano[3,4-c]isoquinolin-1-yl)-N-methylbenzamide (Compound 135)

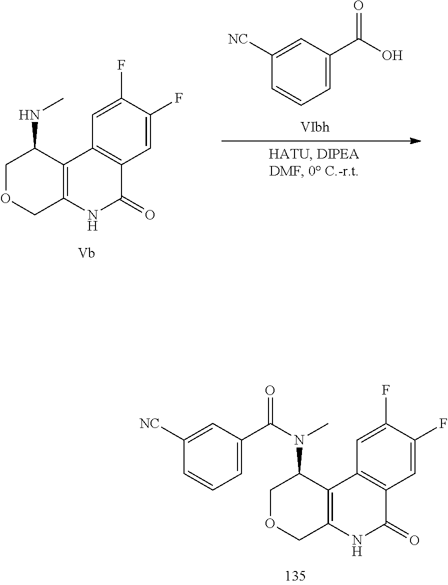 [SMILES: CN[C@@H]1COCc2[nH]c(=O)c3cc(F)c(F)cc3c21 ; N#Cc1cccc(C(=O)O)c1 ; CN(C(=O)c1cccc(C#N)c1)[C@@H]1COCc2[nH]c(=O)c3cc(F)c(F)cc3c21]

Enantiomerically pure (S)-3-cyano-N-(8,9-difluoro-6-oxo-1,4,5,6-tetrahydro-2H-pyrano[3,4-c]isoquinolin-1-yl)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-cyanobenzoic acid (VIbh). LCMS m/z found 396.1 [M+H]⁺; RT=5.42 min (Method A); ¹H NMR (400 MHz, DMSO-d₆) δ 11.7 (br s, 1H), 8.12 (t, 1H), 7.96-7.92 (m, 2H), 7.74-7.65 (m, 2H), 7.5-7.45 (m, 1H), 5.67 (s, 1H), 4.58 (d, 1H), 4.46 (d, 1H), 4.29 (d, 1H), 4.04-4.01 (m, 1H), 2.69 (s, 3H).

### (S)-N-(8,9-Difluoro-6-oxo-1,4,5,6-tetrahydro-2H-pyrano[3,4-c]isoquinolin-1-yl)-3-fluoro-N-methyl-4-(trifluoromethoxy)benzamide (Compound 136)

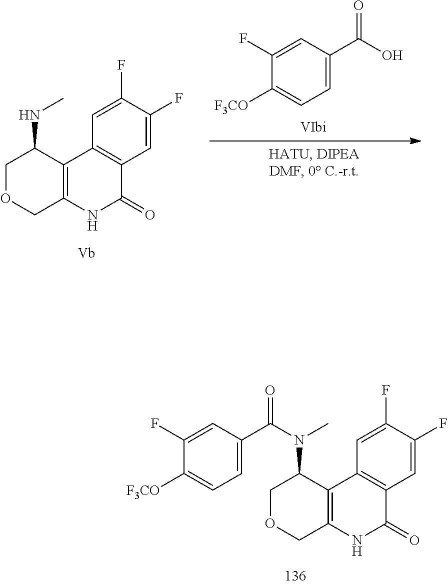 [SMILES: CN[C@@H]1COCc2[nH]c(=O)c3cc(F)c(F)cc3c21 ; O=C(O)c1ccc(OC(F)(F)F)c(F)c1 ; CN(C(=O)c1ccc(OC(F)(F)F)c(F)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-3-fluoro-N-methyl-4-(trifluoromethoxy)benzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-fluoro-4-(trifluoromethoxy)benzoic acid (VIbi). LCMS m/z found 473.1 [M+H]⁺; RT=6.39 min (Method A); ¹H NMR (400 MHz, DMSO-d₆) δ 11.69 (s, 1H), 8.15-8.11 (m, 1H), 7.68-7.65 (m, 2H), 7.48-7.43 (m, 1H), 7.35 (d, 1H), 5.66 (s, 1H), 4.59 (d, 1H), 4.46 (d, 1H), 4.29 (d, 1H), 4.04-4.0 (m, 1H), 2.71 (s, 3H).

### (S)-N-(8,9-Difluoro-6-oxo-1,4,5,6-tetrahydro-2H-pyrano[3,4-c]isoquinolin-1-yl)-3-fluoro-N,4-dimethylbenzamide (Compound 137)

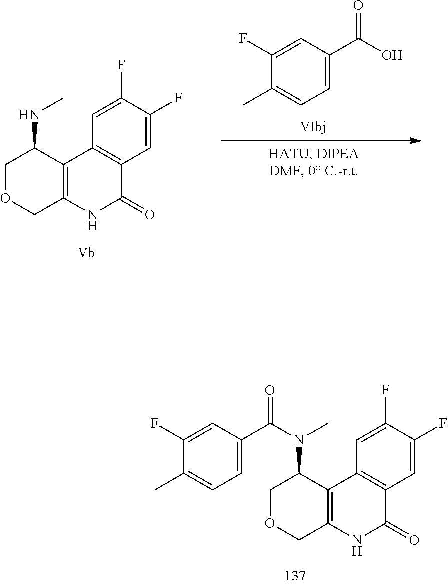 [SMILES: CN[C@@H]1COCc2[nH]c(=O)c3cc(F)c(F)cc3c21 ; Cc1ccc(C(=O)O)cc1F ; Cc1ccc(C(=O)N(C)[C@@H]2COCc3[nH]c(=O)c4cc(F)c(F)cc4c32)cc1F]

Enantiomerically pure (S)-N-(8,9-difluoro-6-oxo-1,4,5,6-tetrahydro-2H-pyrano[3,4-c]isoquinolin-1-yl)-3-fluoro-N,4-dimethylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-fluoro-4-methylbenzoic acid (VIbj). LCMS m/z found 403.2 [M+H]⁺; RT=5.98 min (Method A); ¹H NMR (400 MHz, DMSO-d₆) δ 11.69 (s, 1H), 8.13 (t, 1H), 7.49-7.44 (m, 1H), 7.37 (t, 1H), 7.22 (d, 1H), 7.12 (d, 1H), 5.65 (s, 1H), 4.58 (d, 1H), 4.46 (d, 1H), 4.25 (d, 1H), 4.02 (d, 1H), 2.71 (s, 3H).

### (S)-N-(8,9-Difluoro-6-oxo-1,4,5,6-tetrahydro-2H-pyrano[3,4-c]isoquinolin-1-yl)-4-ethyl-3-fluoro-N-methylbenzamide (Compound 147)

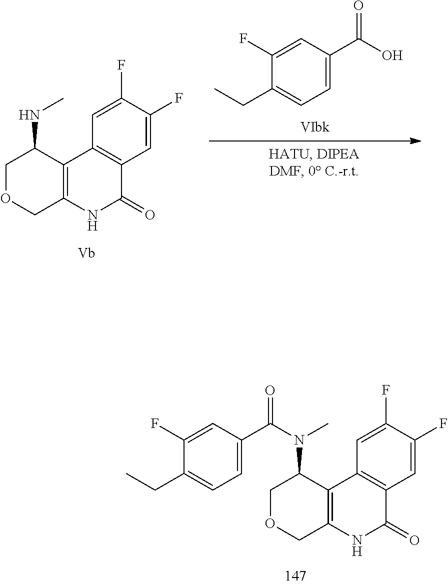

Enantiomerically pure (S)-N-(8,9-difluoro-6-oxo-1,4,5,6-tetrahydro-2H-pyrano[3,4-c]isoquinolin-1-yl)-4-ethyl-3-fluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-ethyl-3-fluorobenzoic acid (VIbk). LCMS m/z found 417.2 [M+H]⁺; RT=6.69 min (Method A); ¹H NMR (400 MHz, DMSO-d₆) δ 11.69 (s, 1H), 8.13 (t, 1H), 7.44-7.35 (m, 2H), 7.15 (d, 2H), 5.66 (s, 1H), 4.58 (d, 1H), 4.46 (d, 1H), 4.2 (d, 1H), 4.04 (d, 1H), 2.64 (s, 3H).

### (S)-N-(8,9-Difluoro-6-oxo-1,4,5,6-tetrahydro-2H-pyrano[3,4-c]isoquinolin-1-yl)-4-(difluoromethoxy)-3-fluoro-N-methylbenzamide (Compound 153)

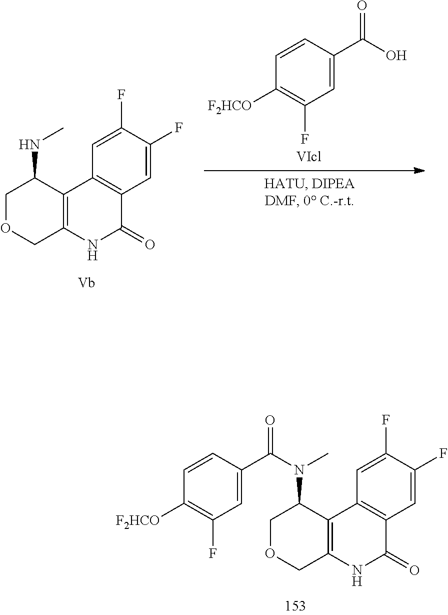 [SMILES: CN[C@@H]1COCc2[nH]c(=O)c3cc(F)c(F)cc3c21 ; O=C(O)c1ccc(OC(F)F)c(F)c1 ; CN(C(=O)c1ccc(OC(F)F)c(F)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-4-(difluoromethoxy)-3-fluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-(difluoromethoxy)-3-fluorobenzoic acid (VIcl). LCMS m/z found 455.3 [M+H]⁺; RT=4.08 min (Method A); ¹H NMR (400 MHz, DMSO-d₆) δ 11.69 (s, 1H), 8.15-8.11 (t, 1H), 7.55 (d, 2H), 7.49-7.44 (m, 1H), 7.36 (t, 1H), 7.1 (s, 1H), 5.66 (s, 1H), 4.62 (d, 1H), 4.46 (d, 1H), 4.26 (d, 1H), 4.04-4.0 (m, 1H), 2.71 (s, 3H).

### (S)-N-(8,9-Difluoro-6-oxo-1,4,5,6-tetrahydro-2H-pyrano[3,4-c]isoquinolin-1-yl)-4-(2-hydroxypropan-2-yl)-N-methylbenzamide (Compound 154)

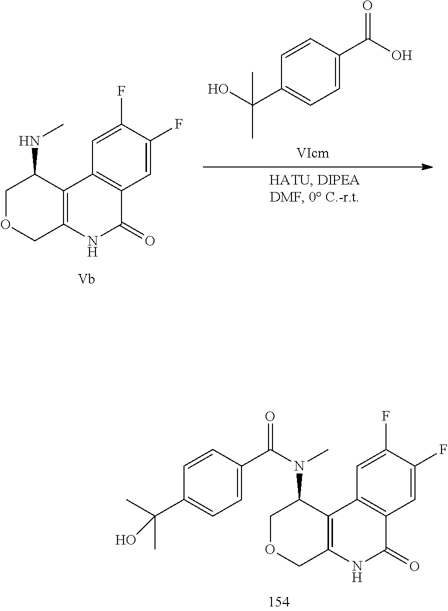 [SMILES: CN[C@@H]1COCc2[nH]c(=O)c3cc(F)c(F)cc3c21 ; CC(C)(O)c1ccc(C(=O)O)cc1 ; CN(C(=O)c1ccc(C(C)(C)O)cc1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-4-(2-hydroxypropan-2-yl)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-(2-hydroxypropan-2-yl)benzoic acid (VIcm). LCMS m/z found 429.3 [M+H]⁺; RT=3.07 min (Method A); ¹H NMR (400 MHz, DMSO-d₆) δ 11.68 (s, 1H), 8.13 (t, 1H), 7.54-7.45 (m, 3H), 7.33 (d, 2H), 5.68 (s, 1H), 5.08 (s, 1H), 4.58 (d, 1H), 4.46 (d, 1H), 4.23 (d, 1H), 4.06-4.02 (m 1H), 2.72 (s, 3H), 1.42 (s, 6H).

### (S)-N-(8,9-Difluoro-6-oxo-1,4,5,6-tetrahydro-2H-pyrano[3,4-c]isoquinolin-1-yl)-3-(2-hydroxypropan-2-yl)-N-methylbenzamide (Compound 155)

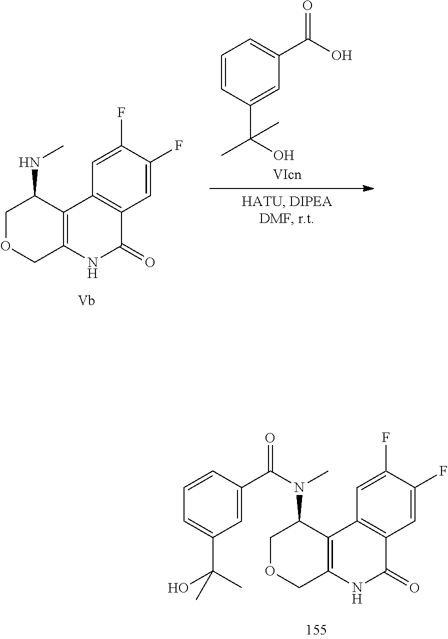

Enantiomerically pure (S)-N-(8,9-difluoro-6-oxo-1,4,5,6-tetrahydro-2H-pyrano[3,4-c]isoquinolin-1-yl)-3-(2-hydroxypropan-2-yl)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-(2-hydroxypropan-2-yl)benzoic acid (VIcn). LCMS m/z found 429.3 [M+H]⁺; RT=4.03 min (Method A); ¹H NMR (400 MHz, DMSO-d₆) δ 11.68 (s, 1H), 8.16-8.11 (m, 1H), 7.52-7.47 (m, 3H), 7.37 (t, 1H), 7.19 (d, 1H), 5.69 (s, 1H), 5.10 (s, 1H), 4.58 (d, 1H), 4.46 (d, 1H), 4.25 (d, 1H), 4.06-4.02 (m, 1H), 2.69 (s, 3H), 1.42 (s, 6H).

### 4-Bromo-N-(8,9-difluoro-6-oxo-2,4,5,6-tetrahydro-1H-pyrano[3,4-c]isoquinolin-1-yl)-3,5-difluoro-N-methylbenzamide (Compound 172)

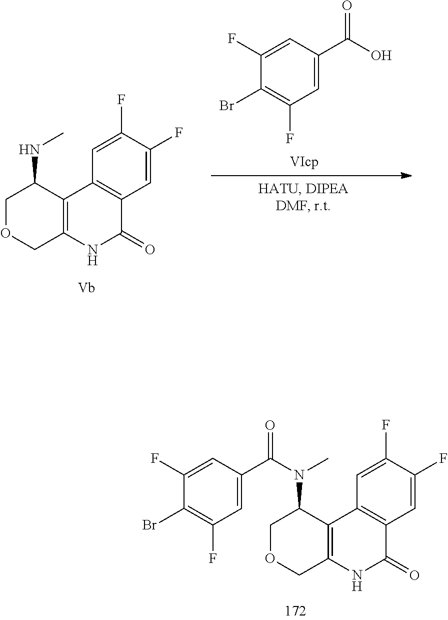 [SMILES: CN[C@@H]1COCc2[nH]c(=O)c3cc(F)c(F)cc3c21 ; O=C(O)c1cc(F)c(Br)c(F)c1 ; CN(C(=O)c1cc(F)c(Br)c(F)c1)[C@@H]1COCc2[nH]c(=O)c3cc(F)c(F)cc3c21]

Enantiomerically pure 4-bromo-N-(8,9-difluoro-6-oxo-2,4,5,6-tetrahydro-1H-pyrano[3,4-c]isoquinolin-1-yl)-3,5-difluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-bromo-3,5-difluorobenzoic acid (VIcp). LCMS m/z found 487.1 [M+H]⁺; RT=8.12 min (Method A); ¹H NMR (400 MHz, DMSO-d₆) δ 11.68 (s, 1H), 8.12 (t, 1H), 7.48-7.43 (m, 1H), 7.38 (d, 2H), 5.62 (s, 1H), 4.60 (d, 1H), 4.44 (d, 1H), 4.30 (d, 1H), 4.01 (dd, 1H), 2.71 (s, 3H).

### 4-Chloro-N-(8,9-difluoro-6-oxo-2,4,5,6-tetrahydro-1H-pyrano[3,4-c]isoquinolin-1-yl)-3,5-difluoro-N-methylbenzamide (Compound 173)

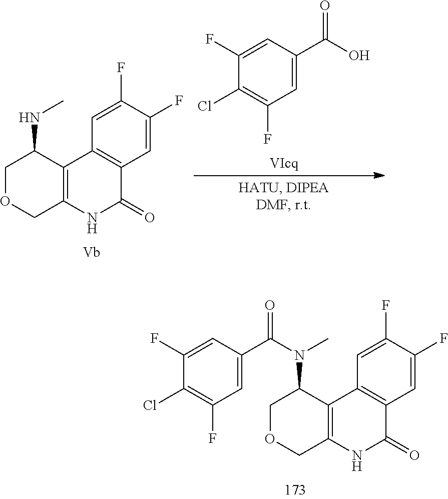

Enantiomerically pure 4-chloro-N-(8,9-difluoro-6-oxo-2,4,5,6-tetrahydro-1H-pyrano[3,4-c]isoquinolin-1-yl)-3,5-difluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-chloro-3,5-difluorobenzoic acid (VIcq). LCMS m/z found 441.3 [M+H]⁺; RT=8.08 min (Method A); ¹H NMR (400 MHz, DMSO-d₆) δ 11.68 (s, 1H), 8.12 (t, 1H), 7.48-7.44 (m, 3H), 5.62 (s, 1H), 4.60 (d, 1H), 4.47 (d, 1H), 4.30 (d, 1H), 4.01 (dd, 1H), 2.71 (s, 3H).

### (S)-N-(8,9-Difluoro-6-oxo-1,4,5,6-tetrahydro-2H-pyrano[3,4-c]isoquinolin-1-yl)-4-(difluoromethyl)-3,5-difluoro-N-methylbenzamide (Compound 180)

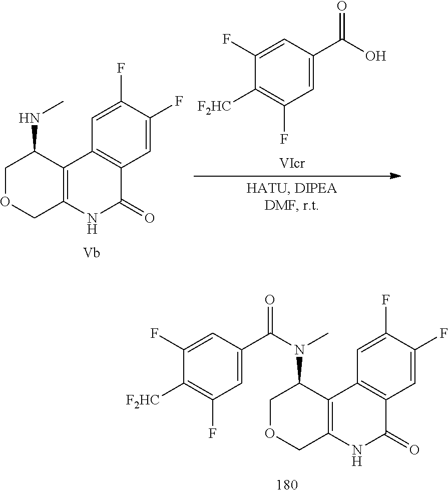 [SMILES: CN[C@@H]1COCc2[nH]c(=O)c3cc(F)c(F)cc3c21 ; O=C(O)c1cc(F)c(C(F)F)c(F)c1 ; CN(C(=O)c1cc(F)c(C(F)F)c(F)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-4-(difluoromethyl)-3,5-difluoro-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-(difluoromethyl)-3,5-difluorobenzoic acid (VIcr). LCMS m/z found 457.3 [M+H]⁺; RT=6.16 min (Method A); ¹H NMR (400 MHz, DMSO-d₆) δ 11.69 (s, 1H), 8.13 (t, 1H), 7.48-7.19 (m, 4H), 5.63 (s, 1H), 4.58 (d, 1H), 4.47 (d, 1H), 4.30 (d, 1H), 4.01 (d, 1H), 2.70 (s, 3H).

### N-((S)-8,9-Difluoro-6-oxo-1,4,5,6-tetrahydro-2H-pyrano[3,4-c]isoquinolin-1-yl)-6-fluoro-4-(1-hydroxyethyl)-N-methyl-1H-indole-2-carboxamide (Compounds 353 and 354)

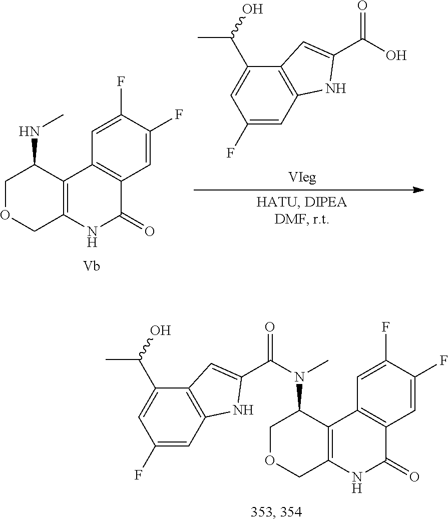 [SMILES: CN[C@@H]1COCc2[nH]c(=O)c3cc(F)c(F)cc3c21 ; CC(O)c1cc(F)cc2[nH]c(C(=O)O)cc12 ; CC(O)c1cc(F)cc2[nH]c(C(=O)N(C)[C@@H]3COCc4[nH]c(=O)c5cc(F)c(F)cc5c43)cc12]

N-((S)-8,9-difluoro-6-oxo-1,4,5,6-tetrahydro-2H-pyrano[3,4-c]isoquinolin-1-yl)-6-fluoro-4-(1-hydroxyethyl)-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and racemic 6-fluoro-4-(1-hydroxyethyl)-1H-indole-2-carboxylic acid (VIeg). The diastereoisomers were subsequently separated by chiral preparative SFC: method isocratic, mobile phase methanol: CO₂—30:70. Column: Chiralpak-OJ (30×250 mm), 5 μm, flow rate: 60 g/min.

Diastereoisomer I (Compound 353): LCMS: m/z found 472.1 [M+H]⁺, RT=3.45 min, (Method A); ¹H NMR (400 MHz, DMSO-d₆) δ 11.77 (br s, 1H), 11.71 (s, 1H), 8.12 (t, 1H), 7.47 (t, 1H), 7.04 (d, 2H), 6.93 (d, 1H), 5.76 (s, 1H), 5.29 (d, 1H), 5.11 (t, 1H), 4.64 (d, 1H), 4.48 (d, 1H), 4.17 (d, 1H), 4.04 (d, 1H), 3.17 (s, 3H), 1.39 (d, 3H); Chiral analytical SFC: RT=1.58 min, Column: Chiralcel OJ-3 (4.6×150 mm) 3 μm, 30% Methanol, Flow rate: 3.0 g/min.

Diastereoisomer II (Compound 354): LCMS: m/z found 472.1 [M+H]⁺, RT=3.52 min, (Method A); ¹H NMR (400 MHz, DMSO-d₆) δ 11.77 (br s, 1H), 11.71 (s, 1H), 8.12 (t, 1H), 7.45 (t, 1H), 7.04 (d, 2H), 6.92 (d, 1H), 5.76 (s, 1H), 5.29 (d, 1H), 5.11 (t, 1H), 4.64 (d, 1H), 4.48 (d, 1H), 4.17 (d, 1H), 4.04 (d, 1H), 3.17 (s, 3H), 1.38 (d, 3H); Chiral analytical SFC: RT=2.97 min, Column: Chiralcel OJ-3 (4.6×150 mm) 3 μm, 30% Methanol, Flow rate: 3.0 g/min.

### (S)-N-(8,9-Difluoro-6-oxo-1,4,5,6-tetrahydro-2H-pyrano[3,4-c]isoquinolin-1-yl)-N-methyl-2-phenylacrylamide (Compound 189)

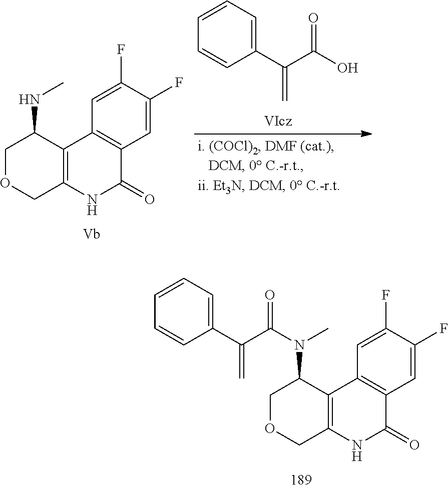

To a stirred solution of 39 mg (0.26 mmol, 1.4 eq.) of 2-phenylacrylic acid (VIcz) in 2 mL of DCM was added 0.05 mL (0.56 mmol, 3 eq.) of oxalylchloride and a catalytic amount of DMF at 0° C. and the reaction mixture was stirred at room temperature for 4 h. The reaction mixture was evaporated to dryness. The obtained acid chloride was taken in 2 mL of DCM and 0.08 mL (0.56 mmol, 3 eq.) of TEA were added, followed by 50 mg (0.19 mmol, 1 eq.) of 8,9-difluoro-1-(methylamino)-1,5-dihydro-2H-pyrano[3,4-c]isoquinolin-6(4H)-one (Vb) at 0° C. and the reaction was stirred at room temperature for 16 h. After completion of reaction, the mixture was poured into ice cold water (30 mL) and stirred for 30 min. The solid formed from the reaction was collected by filtration and dried under vacuum. The obtained material was purified by preparative HPLC [Column/dimensions: X-BRIDGE PHENYLE (19×250, 5 μm) Mobile phase A: 10 mM Ammonium Bicarbonate in water Mobile phase B: Acetonitrile Gradient (Time/% B): 0/25, 1/25, 8/55, 12/55, 12.1/100, 16/100, 16.1/25, 18/25. Flow rate: 18 ml/min] to afford 20 mg (0.05 mmol, 26% yield) of N-(8,9-difluoro-6-oxo-1,4,5,6-tetrahydro-2H-pyrano[3,4-c]isoquinolin-1-yl)-4-(difluoromethyl)-N-methyl-1H-indole-2-carboxamide, as an off-white solid. LCMS m/z found 397.3 [M+H]⁺; RT=4.36 min (Method A); ¹H NMR (400 MHz, DMSO-d₆) δ 11.67 (s, 1H), 8.14-8.09 (m, 1H), 7.49-7.44 (m, 3H), 7.40-7.34 (m, 3H), 5.85 (s, 1H), 5.69 (s, 1H), 5.32 (s, 1H), 4.57 (d, 1H), 4.45 (d, 1H), 4.20 (d, 1H), 4.04 (dd, 1H), 2.68 (s, 3H).

### 2-Amino-2-(4-chlorophenyl)-N-((S)-8,9-difluoro-6-oxo-1,4,5,6-tetrahydro-2H-pyrano[3,4-c]isoquinolin-1-yl)-N-methylacetamide (Compounds 199 and 200)

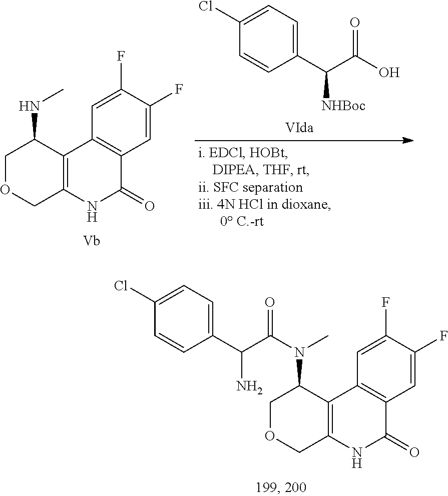 [SMILES: CN[C@@H]1COCc2[nH]c(=O)c3cc(F)c(F)cc3c21 ; CC(C)(C)OC(=O)N[C@H](C(=O)O)c1ccc(Cl)cc1 ; CN(C(=O)C(N)c1ccc(Cl)cc1)[C@@H]1COCc2[nH]c(=O)c3cc(F)c(F)cc3c21]

Step i. To a stirred solution of 128 mg (0.375 mmol, 1.2 eq.) of (S)-2-((tert-butoxycarbonyl)amino)-2-(4-chlorophenyl)acetic acid (VIda) in 2 mL of THE at room temperature were added 0.2 mL (1.12 mmol, 3 eq.) of DIPEA, 87 mg (0.56 mmol, 1.5 eq.) of EDCI, followed by 76 mg (0.56 mmol, 1.5 eq.) of HOBt and the reaction mixture was stirred at room temperature for 15 min. (S)-8,9-Difluoro-1-(methylamino)-1,5-dihydro-2H-pyrano[3,4-c]isoquinolin-6(4H)-one (Vb, 100 mg, 0.495 mmol, 1 eq.) was added to the reaction mixture and stirring was continued for 16 h. After completion of reaction, the reaction mixture was poured into ice cold saturated NaHCO₃ solution (10 mL), stirred for 30 min, when a solid precipitated. The solid was collected by filtration, washed with water, and dried under vacuum. Column chromatography (using 30% ethyl acetate in petroleum ether as a linear gradient) afforded racemic 85 mg (0.15 mmol, 42% yield) of tert-butyl (1-(4-chlorophenyl)-2-(((S)-8,9-difluoro-6-oxo-1,4,5,6-tetrahydro-2H-pyrano[3,4-c]isoquinolin-1-yl)(methyl)amino)-2-oxoethyl)carbamate as an off white solid. LCMS m/z found 532.37 [M−H]⁻.

Step ii. The diastereoisomers of tert-butyl (1-(4-chlorophenyl)-2-(((S)-8,9-difluoro-6-oxo-1,4,5,6-tetrahydro-2H-pyrano[3,4-c]isoquinolin-1-yl)(methyl)amino)-2-oxoethyl)carbamate were subsequently separated by chiral preparative SFC: method isocratic, mobile phase methanol:CO₂—30:70. Column: Lux Cellulose-2 (30×250 mm), 5 μm, flow rate: 110 g/min.

Step iii. Each individual diastereoisomer of tert-butyl (1-(4-chlorophenyl)-2-(((S)-8,9-difluoro-6-oxo-1,4,5,6-tetrahydro-2H-pyrano[3,4-c]isoquinolin-1-yl)(methyl)amino)-2-oxoethyl)carbamate, isolated as described above, was converted to the final product, by treatment with 4N HCl in dioxane at 0° C., followed by stirring at room temperature for 10 h, removal of the volatiles under reduced pressure. The resulting residue from each reaction was taken in saturated NaHCO₃ solution and stirred for 10 min. The precipitated solids were collected by filtration and the products were further purified by trituration with diethyl ether and filtration, and dried under high vacuum.

Diastereoisomer I (Compound 199): LCMS: m/z found 434.2 [M+H]⁺, RT=3.73 min, (Method A); ¹H NMR (400 MHz, DMSO-d₆) δ): δ 11.5 (bs, 1H), 8.13-8.08 (m, 11H1H), 7.59-7.28 (m, 5H), 5.58 (s, 1H), 4.85 (s, 1H), 4.58-4.39 (m, 2H), 4.24-4.04 (m, 2H) 2.67 (s, 3H), 2.60 (bs, 2H); Chiral analytical SFC: RT=2.67 min, Column: Chiralcel OX-3 (4.6×150 mm) 3 μm, 20% (0.5% of DEA in Methanol), Flow rate: 3.0 g/min.

Diastereoisomer II (Compound 200): LCMS: m/z found 434.2 [M+H]⁺, RT=3.67 min, (Method A); ¹H NMR (400 MHz, DMSO-d₆) δ): δ 11.50 (bs, 1H), 8.04-7.30 (m, 5H), 6.84-6.79 (m, 1H), 5.49 (s, 1H), 4.88 (s, 1H), 4.57-4.38 (m, 2H), 4.05-3.92 (m, 2H) 2.76 (s, 3H), 2.59 (bs, 2H); Chiral analytical SFC: RT=4.14 min, Column: Chiralcel OX-3 (4.6×150 mm) 3 μm, 20% (0.5% of DEA in Methanol), Flow rate: 3.0 g/min.

### 2-Amino-2-(3-chlorophenyl)-N-((S)-8,9-difluoro-6-oxo-1,4,5,6-tetrahydro-2H-pyrano[3,4-c]isoquinolin-1-yl)-N-methylacetamide (Compounds 202 and 203)

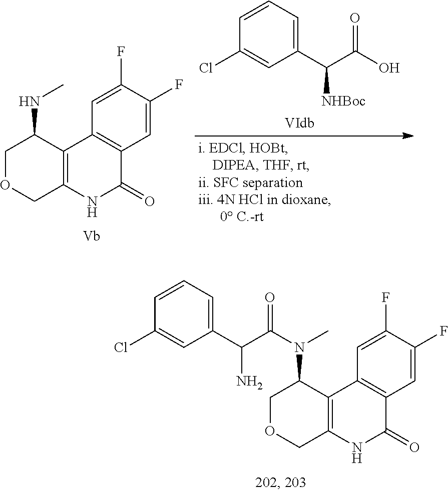

Individual diastereoisomers of 2-amino-2-(3-chlorophenyl)-N-((S)-8,9-difluoro-6-oxo-1,4,5,6-tetrahydro-2H-pyrano[3,4-c]isoquinolin-1-yl)-N-methylacetamide were prepared in an analogous manner as described above (for Compounds 199, 200), from (S)-8,9-difluoro-1-(methylamino)-1,5-dihydro-2H-pyrano[3,4-c]isoquinolin-6(4H)-one (Vb), and (S)-2-((tert-butoxycarbonyl)amino)-2-(3-chlorophenyl)acetic acid (VIdb).

Diastereoisomer I (Compound 202): LCMS: m/z found 434.4 [M+H]⁺, RT=4.86 min, (Method A); ¹H NMR (400 MHz, DMSO-d₆) δ 11.28 (bs, 1H), 8.06 (bs, 1H), 7.44-7.33 (m, 5H), 5.59 (s, 1H), 4.94 (s, 1H), 4.528 (d, 1H), 4.38 (d, 1H), 3.89-3.78 (m, 2H), 2.70 (s, 3H), 2.60 (bs, 2H); Chiral analytical SFC: RT=2.49 min, Column: Chiralcel OX-3 (4.6×150 mm) 3 μm, 40% (0.2% 7M Methanolic ammonia in Acetonitrile: Methanol)(1:1), Flow rate: 3.0 g/min.

Diastereoisomer II (Compound 203): LCMS: m/z found 434.4 [M+H]⁺, RT=5.96 min, (Method A); ¹H NMR (400 MHz, DMSO-d₆) δ 11.67 (bs, 1H), 8.04 (bs, 1H), 7.38-7.28 (m, 4H), 6.96 (t, 1H), 5.54 (s, 1H), 4.88 (s, 1H), 4.54 (d, 1H), 4.40 (d, 1H), 4.05 (s, 1H), 3.95 (d, 1H), 2.78 (s, 3H), 2.35-2.07 (m, 2H); Chiral analytical SFC: RT=3.80 min, Column: Chiralcel OX-3 (4.6×150 mm) 3 μm, 40% (0.2% 7M Methanolic ammonia in Acetonitrile:Methanol)(1:1), Flow rate: 3.0 g/min.

### (S)-1-Amino-8,9-difluoro-1,5-dihydro-2H-pyrano[3,4-c]isoquinolin-6(4H)-one (Vo)

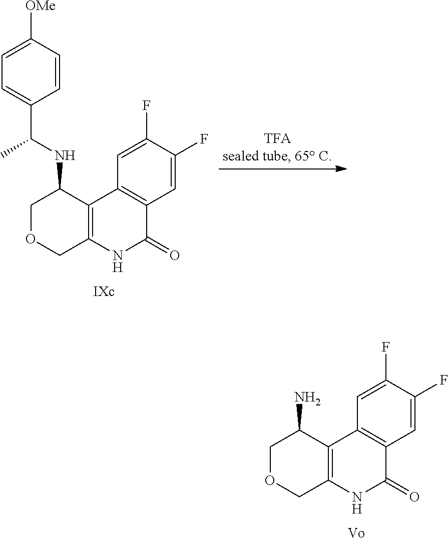 [SMILES: COc1ccc([C@@H](C)N[C@@H]2COCc3[nH]c(=O)c4cc(F)c(F)cc4c32)cc1 ; N[C@@H]1COCc2[nH]c(=O)c3cc(F)c(F)cc3c21]

In a sealed tube, to 250 mg (0.647 mmol, 1.1 eq.) of (S)-8,9-difluoro-1-(((R)-1-(4-methoxyphenyl)ethyl)amino)-1,5-dihydro-2H-pyrano[3,4-c]isoquinolin-6(4H)-one (IXc) was added 3.7 mL (15 vol) of trifluoroacetic acid. The reaction mixture was stirred at 65° C. ° C. ° C. for 2 h. After completion of the reaction (by TLC), the reaction mixture was concentrated under reduced pressure and triturated with diethyl ether (2×20 mL). Obtained crude material was basified with 10% aq. Na₂CO₃ solution and extracted with EtOAc (2×60 mL). Organic extract was washed with water (30 mL), brine (30 mL), dried over anhydrous Na₂SO₄ and concentrated under reduced pressure to afford (S)-1-amino-8,9-difluoro-1,5-dihydro-2H-pyrano[3,4-c]isoquinolin-6(4H)-one (135 mg, 82% yield) as white solid. LCMS m/z found 251.33 [M−H]⁻, RT=2.24 min (Method E).

### (S)-N-(8,9-Difluoro-6-oxo-1,4,5,6-tetrahydro-2H-pyrano[3,4-c]isoquinolin-1-yl)indolizine-2-carboxamide (Compound 148)

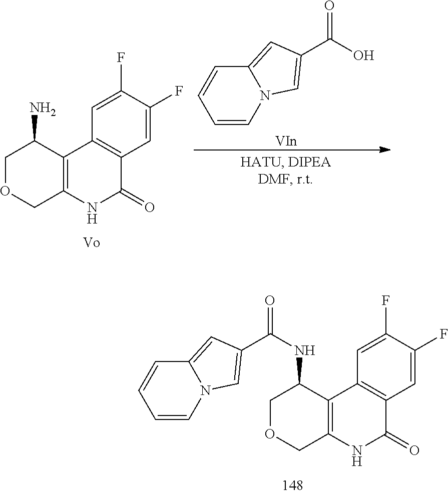

Enantiomerically pure (S)-N-(8,9-difluoro-6-oxo-1,4,5,6-tetrahydro-2H-pyrano[3,4-c]isoquinolin-1-yl)indolizine-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vo) and indolizine-2-carboxylic acid (VIn). LCMS m/z found 396.1 [M+H]⁺; RT=6.07 min (Method A); ¹H NMR (400 MHz, DMSO-d₆) δ 11.60 (s, 1H), 8.57 (d, 1H), 8.21 (d, 1H), 8.12-8.05 (m, 2H), 7.47-7.475 (m, 2H), 6.85 (s, 1H), 6.72-6.68 (m, 1H), 6.58 (t, 1H), 5.20 (d, 1H), 4.55-4.45 (m, 2H), 4.01 (d, 1H), 3.88 (d, 1H).

### (S)-N-(8,9-Difluoro-6-oxo-1,4,5,6-tetrahydro-2H-pyrano[3,4-c]isoquinolin-1-yl)-5,6-difluoro-1H-indole-2-carboxamide (Compound 156)

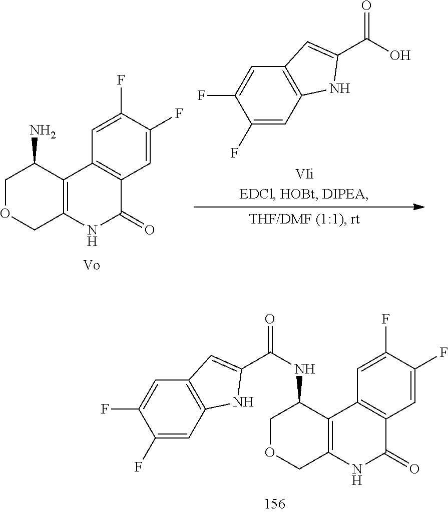

To a stirred solution of 42 mg (0.22 mmol, 1.1 eq.) of 5,6-difluoro-1H-indole-2-carboxylic acid (VIi) in 0.75 mL of THF at room temperature were added 0.17 mL (1 mmol, 5 eq.) of DIPEA, 113 mg (0.6 mmol, 3 eq.) of EDCI, and 80 mg of (0.6 mmol, 3 eq.) of HOBt and the reaction mixture was stirred at room temperature for 15 min. To this mixture 50 mg (0.2 mmol, 1 eq.) of (S)-1-amino-8,9-difluoro-1,5-dihydro-2H-pyrano[3,4-c]isoquinolin-6(4H)-one (Vo) was added and the resulting mixture was stirred for 2 h. After completion of reaction (by TLC), the reaction mixture was poured on to ice-water (10 mL) and extracted with EtOAc (2×30 mL). Organic layer was washed with water (20 mL), dried over Na₂SO₄, and concentrated under reduced pressure. Obtained crude material was triturated with water (10 mL) and diethyl ether/n-pentane (1:1, 2×10 ml) to afford (S)-N-(8,9-difluoro-6-oxo-1,4,5,6-tetrahydro-2H-pyrano[3,4-c]isoquinolin-1-yl)-5,6-difluoro-1H-indole-2-carboxamide (24 mg, 28% yield) as white solid. LCMS m/z found 432.3 [M+H]⁺; RT=4.80 min (Method A); ¹H NMR (400 MHz, DMSO-d₆) δ 11.75 (bs, 2H), 8.94 (d, 1H), 8.09 (t, 1H), 7.63-7.58 (m, 1H), 7.48-7.43 (m, 1H), 7.36-7.32 (m, 1H), 7.22 (s, 1H), 5.21 (d, 1H), 4.56-4.61 (m, 2H), 4.04 (d, 1H), 3.93-3.90 (m, 1H).

### (S)-N-(8,9-Difluoro-6-oxo-1,4,5,6-tetrahydro-2H-pyrano[3,4-c]isoquinolin-1-yl)-4,6-difluoro-1H-indole-2-carboxamide (Compound 157)

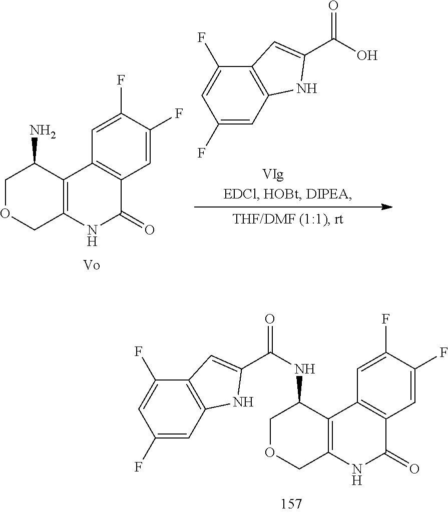

Enantiomerically pure (S)-N-(8,9-difluoro-6-oxo-1,4,5,6-tetrahydro-2H-pyrano[3,4-c]isoquinolin-1-yl)-4,6-difluoro-1H-indole-2-carboxamide was synthesized in an analogous manner as described above from enantiomerically pure (S)-1-amino-8,9-difluoro-1,5-dihydro-2H-pyrano[3,4-c]isoquinolin-6(4H)-one (Vo) and 4,6-difluoro-1H-indole-2-carboxylic acid (VIg). LCMS m/z found 432.3 [M+H]⁺; RT=4.24 min (Method A); ¹H NMR (400 MHz, DMSO-d₆) δ 12.1 (s, 1H), 11.64 (s, 1H), 8.97 (d, 1H), 8.13-8.08 (m, 1H), 7.48-7.44 (m, 1H), 7.32 (s, 1H), 7.03 (d, 1H), 6.86 (t, 1H), 5.22 (d, 1H), 4.57-4.47 (m, 2H), 4.06 (d, 1H), 3.92 (d, 1H).

### (S)-N-(8,9-Difluoro-6-oxo-1,4,5,6-tetrahydro-2H-pyrano[3,4-c]isoquinolin-1-yl)-3-fluoro-4-(trifluoromethyl)benzamide (Compound 158)

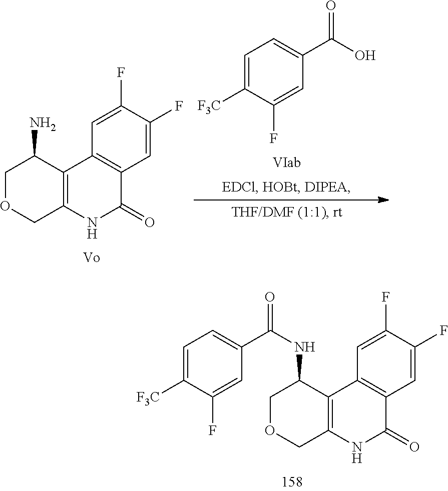

Enantiomerically pure (S)-N-(8,9-difluoro-6-oxo-1,4,5,6-tetrahydro-2H-pyrano[3,4-c]isoquinolin-1-yl)-3-fluoro-4-(trifluoromethyl)benzamide was synthesized in an analogous manner as described above from enantiomerically pure (S)-1-amino-8,9-difluoro-1,5-dihydro-2H-pyrano[3,4-c]isoquinolin-6(4H)-one (Vo) and 3-fluoro-4-(trifluoromethyl)benzoic acid (VIab). LCMS m/z found 441 [M−H]⁻; RT=3.06 min (Method C); ¹H NMR (400 MHz, DMSO-d₆) δ 11.67 (s, 1H), 9.19 (d, 1H), 8.11 (t, 1H), 7.95-7.88 (m, 3H), 7.42-7.38 (m, 1H), 5.19 (d, 1H), 4.51 (m, 2, H), 4.08 (d, 1H), 3.91 (d, 1H).

### (S)-4-chloro-N-(8,9-difluoro-6-oxo-1,4,5,6-tetrahydro-2H-pyrano[3,4-c]isoquinolin-1-yl)-3-fluorobenzamide (Compound 159)

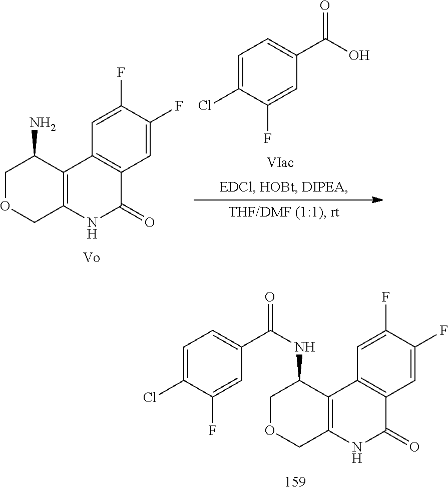

Enantiomerically pure (S)-4-chloro-N-(8,9-difluoro-6-oxo-1,4,5,6-tetrahydro-2H-pyrano[3,4-c]isoquinolin-1-yl)-3-fluorobenzamide was synthesized in an analogous manner as described above from enantiomerically pure (S)-1-amino-8,9-difluoro-1,5-dihydro-2H-pyrano[3,4-c]isoquinolin-6(4H)-one (Vo) and 4-chloro-3-fluorobenzoic acid (VIac). LCMS m/z found 409.2 [M+H]⁺; RT=4.94 min (Method A); ¹H NMR (400 MHz, DMSO-d₆) δ 11.63 (s, 1H), 9.02 (d, 1H), 8.12-8.08 (m, 1H), 7.92-7.89 (m, 1H), 7.79-7.76 (m, 1H), 7.69 (t, 1H), 7.40-7.36 (m, 1H), 5.17 (d, 1H), 4.54-4.45 (m, 2H), 4.05 (d, 1H), 3.91-3.88 (m, 1H).

### (S)-N-(8,9-Difluoro-6-oxo-1,4,5,6-tetrahydro-2H-pyrano[3,4-c]isoquinolin-1-yl)-6-fluoro-N-methylindolizine-2-carboxamide (Compound 224)

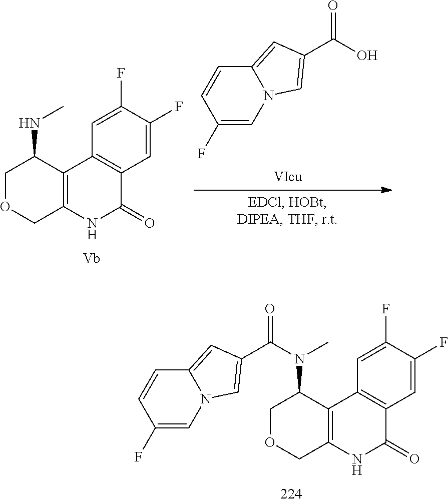

Enantiomerically pure (S)-N-(8,9-difluoro-6-oxo-1,4,5,6-tetrahydro-2H-pyrano[3,4-c]isoquinolin-1-yl)-6-fluoro-N-methylindolizine-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 6-fluoroindolizine-2-carboxylic acid (VIcu). LCMS m/z found 428.3 [M+H]⁺; RT=5.17 min (Method A); ¹H NMR (400 MHz, DMSO-d₆) δ 11.67 (s, 1H), 8.45 (bs, 1H), 8.14-8.10 (m, 1H), 7.93 (s, 1H), 7.54-7.41 (m, 2H), 6.86-6.81 (m, 1H), 6.75 (s, 1H), 5.71 (s, 1H), 4.63 (d, 1H), 4.48 (d, 1H), 4.17 (d, 1H), 4.04 (d, 1H), 3.02 (s, 3H).

### (S)-N-(8,9-Difluoro-6-oxo-1,4,5,6-tetrahydro-2H-pyrano[3,4-c]isoquinolin-1-yl)-4-(difluoromethyl)-6-fluoro-N-methyl-1H-indole-2-carboxamide (Compound 230)

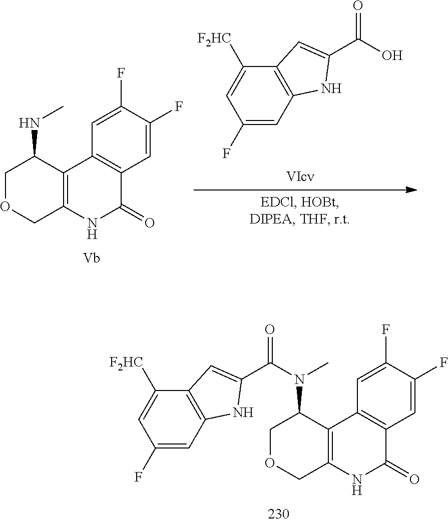 [SMILES: CN[C@@H]1COCc2[nH]c(=O)c3cc(F)c(F)cc3c21 ; O=C(O)c1cc2c(C(F)F)cc(F)cc2[nH]1 ; CN(C(=O)c1cc2c(C(F)F)cc(F)cc2[nH]1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-4-(difluoromethyl)-6-fluoro-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-(difluoromethyl)-6-fluoro-1H-indole-2-carboxylic acid (VIcv). LCMS m/z found 478.3 [M+H]⁺; RT=6.79 min (Method A); ¹H NMR (400 MHz, DMSO-d₆) δ 12.16 (bs, 1H), 11.72 (bs, 1H), 8.15-8.10 (m, 1H), 7.48-7.19 (m, 2H), 7.07 (s, 1H), 6.76 (s, 1H), 5.75 (s, 1H), 4.66 (d, 1H), 4.51 (d, 1H), 4.21 (d, 1H), 4.06 (d, 1H), 3.66 (bs, 1H), 3.15 (s, 3H).

### 7-Fluoroindolizine-2-carboxylic acid (VIco)

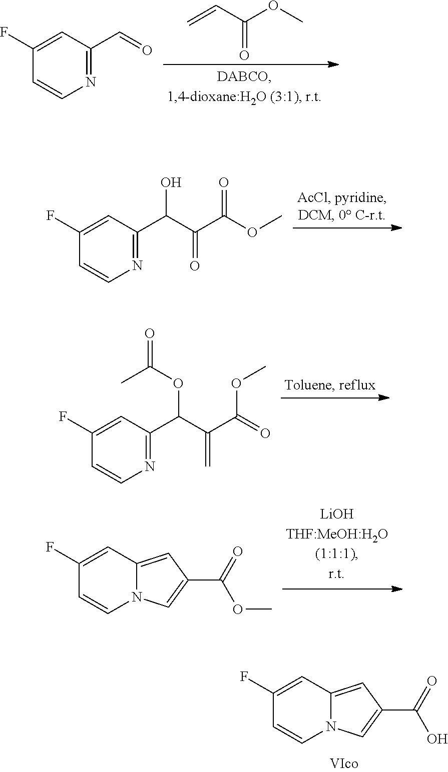

Step i. To a stirred solution of 4-fluoropicolinaldehyde in 20 mL of 1,4-dioxane and 10 mL of water at room temperature, 0.53 mL (5.91 mmol, 1 eq.) of methyl acrylate, 40 mg (0.36 mmol, 0.06 eq.) of DABCO were added and then the reaction was stirred at room temperature for 16 h. After completion of reaction the mixture was diluted with water (50 mL) and extracted with EtOAc (3×50 mL). The combined organic layer was dried over anhydrous Na₂SO₄ and concentrated under reduced pressure to afford 410 mg of crude compound. The crude product was purified by Combi-flash chromatography (silica gel) using 30-40% of ethyl acetate in petroleum ether as eluent to afford 250 mg (20% yield) of methyl 2-((4-fluoropyridin-2-yl)(hydroxy)methyl) acrylate as off-white solid. LCMS m/z found 212.16 [M+H]⁺; ¹H NMR (400 MHz, CDCl₃) δ 8.48 (q, 1H), 7.33-7.30 (m, 1H), 7.21-7.17 (m, 1H), 6.20 (s, 1H), 6.07 (d, 1H), 5.87 (t, 1H), 5.51 (s, 1H), 3.61 (s, 3H). Note: Reaction was repeated on 1 g scale as described above and obtained consistent results.

Step ii. To a solution of 700 mg (3.31 mmol, 1 eq.) of methyl 2-((4-fluoropyridin-2-yl)(hydroxy)methyl) acrylate in DCM (7 mL) was added 0.4 mL (4.97 mmol, 1.5 eq.) pyridine, and 355 mg (4.97 mmol, 1.5 eq.) of AcCl dropwise at 0° C. and reaction was stirred at r.t. for 1 h. After completion of reaction the mixture was poured in a saturated NaHCO₃ solution (50 mL) and extracted with EtOAc (3×50 mL). The combined organic layer was dried over anhydrous Na₂SO₄ and concentrated under reduced pressure to afford 0.7 g (crude) of methyl 2-(acetoxy(4-fluoropyridin-2-yl)methyl)acrylate as a yellow oil. The obtained crude compound was taken as such into the next step.

Step iii. A solution of 700 mg (crude) (2.76 mmol, 1 eq.) of methyl 2-(acetoxy(4-fluoropyridin-2-yl)methyl)acrylate in toluene (14 mL) was heated to reflux for 16 h. After completion of reaction the mixture was evaporated to dryness. The obtained crude compound was mixed with another batch of same quantity and purified by Combi-flash chromatography (silica gel) using 0-40% of ethyl acetate in petroleum ether as eluent to afford 300 mg (23% yield) of methyl 7-fluoroindolizine-2-carboxylate as an off-white solid. LCMS m/z found 194.07 [M+H]⁺; ¹H NMR (400 MHz, DMSO-d₆) δ 8.36 (t, 1H), 8.09 (s, 1H), 7.28 (dd, 1H), 6.76-6.72 (m, 1H), 6.68 (s, 1H), 3.79 (s, 3H).

Step iv. To a stirred solution of 450 mg (2.33 mmol, 1 eq.) of methyl 7-fluoroindolizine-2-carboxylate in a mixture of THF:Water:MeOH (2:1:1) (9 mL), 279 mg (11.67 mmol, 5 eq.) of lithium hydroxide was added and the reaction was stirred at room temperature for 16 h. After completion of reaction the mixture was evaporated to dryness. The obtained residue was taken in 10% KHSO₄ solution (10 mL) and stirred for 10 minutes. The precipitated solids were collected by filtration and dried under vacuum to afford 210 mg (50% yield) of 7-fluoroindolizine-2-carboxylic acid (VIco) as a brown solid. LCMS m/z found 180.17 [M+H]⁺; ¹H NMR (400 MHz, DMSO-d₆) δ 8.35 (t, 1H), 8.10 (s, 1H), 7.26 (dd, 1H), 6.73-6.68 (m, 1H), 6.64 (s, 1H).

### (S)-N-(8,9-Difluoro-6-oxo-1,4,5,6-tetrahydro-2H-pyrano[3,4-c]isoquinolin-1-yl)-7-fluoro-N-methylindolizine-2-carboxamide (Compound 231)

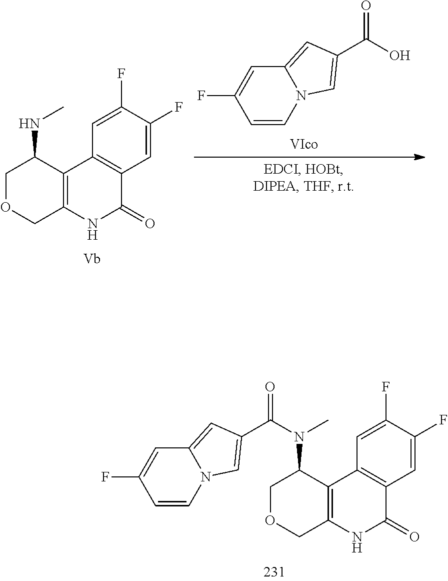

Enantiomerically pure (S)-N-(8,9-difluoro-6-oxo-1,4,5,6-tetrahydro-2H-pyrano[3,4-c]isoquinolin-1-yl)-7-fluoro-N-methylindolizine-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 7-fluoroindolizine-2-carboxylic acid (VIco). LCMS m/z found 428.3 [M+H]⁺; RT=6.23 min (Method A); ¹H NMR (400 MHz, DMSO-d₆) δ 11.67 (s, 1H), 8.32 (bs, 1H), 8.14-8.10 (m, 1H), 7.90 (s, 1H), 7.43-7.21 (m, 2H), 6.68-6.58 (m, 2H), 5.75 (s, 1H), 4.63 (d, 1H), 4.48 (d, 1H), 4.17 (d, 1H), 4.04 (d, 1H), 3.00 (s, 3H).

### (S)-N-(8,9-Difluoro-6-oxo-1,4,5,6-tetrahydro-2H-pyrano[3,4-c]isoquinolin-1-yl)-4-(difluoromethyl)-5-fluoro-N-methyl-1H-indole-2-carboxamide (Compound 254)

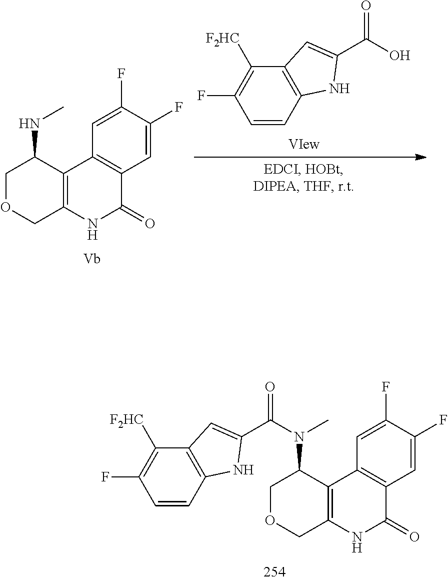

Enantiomerically pure (S)-N-(8,9-difluoro-6-oxo-1,4,5,6-tetrahydro-2H-pyrano[3,4-c]isoquinolin-1-yl)-4-(difluoromethyl)-5-fluoro-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-(difluoromethyl)-5-fluoro-1H-indole-2-carboxylic acid (VIcw). LCMS m/z found 478.3 [M+H]⁺; RT=7.68 min (Method A); ¹H NM/R (400 MHz, DMSO-d₆) δ 12.18 (bs, 1H), 11.73 (bs, 1H), 8.15-8.10 (m, 1H), 7.66-7.65 (i, 1H), 7.59-7.32 (m, 2H) 7.22-7.17 (t, 1H), 7.07 (s, 1H), 5.75 (s, 1H), 4.66 (d, 1H), 4.51 (d, 1H), 4.22 (d, 1H), 4.06 (d, 1H), 3.14 (s, 3H).

### 5-(Difluoromethyl)indolizine-2-carboxylic acid (VIdc)

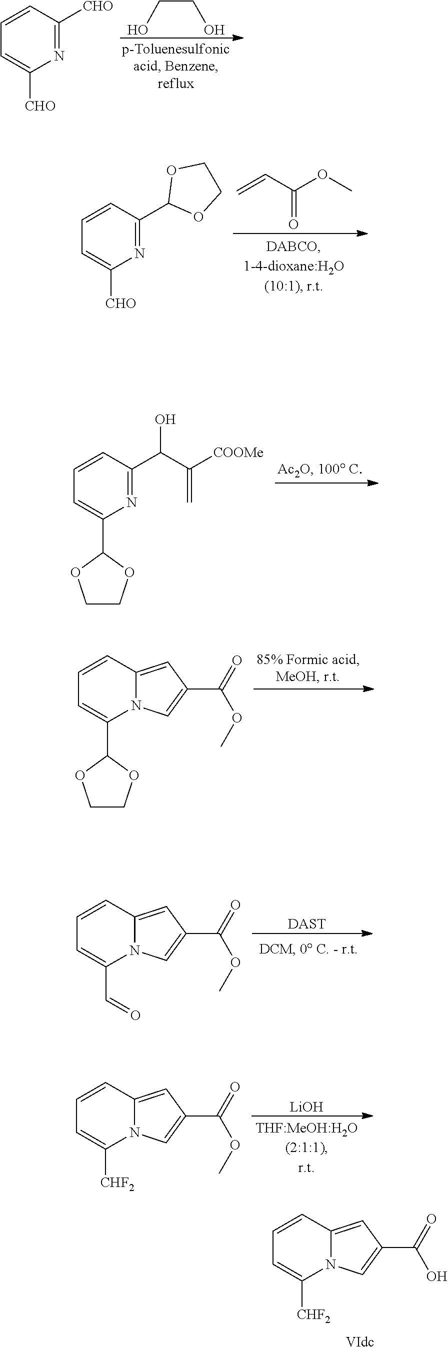

Step i. To a stirred solution 10 g (74.1 mmol, 1 eq.) of pyridine-2,6-dicarbaldehyde in 100 mL of benzene at room temperature, 4.1 mL (66.6 mmol, 0.9 eq.) of ethane-1,2-diol, 1.2 g (7.4 mmol, 0.1 eq.) of p-toluenesulfonic acid were added and then the reaction was stirred at 100° C. for 4 h, using a Dean-Stark apparatus to remove water. After completion of reaction, the mixture was diluted with water (50 mL) and extracted with EtOAc (3×50 mL).

The combined organic layer was dried over anhydrous Na₂SO₄ and concentrated under reduced pressure to afford 13 g of crude compound. The material was purified by Combi-flash (silica gel) using 30-40% of ethyl acetate in petroleum ether as eluent to afford 3.4 g (26% yield) of 6-(1,3-dioxolan-2-yl)picolinaldehyde as an off-white solid. LCMS m/z found 180.31 [M+H]⁺, RT=1.13 min, (Method E).

Step ii. To a stirred solution of 3.5 g of 6-(1,3-dioxolan-2-yl)picolinaldehyde in 35 mL of 1,4-dioxane and 3.5 mL of water at room temperature, 0.53 mL (5.9 mmol, 1 eq.) of methyl acrylate, 3 g (0.36 mmol, 0.06 eq.) of DABCO were added and then the reaction mixture was stirred at room temperature for 16 h. After completion of reaction, the mixture was diluted with water (50 mL) and extracted with EtOAc (3×50 mL). The combined organic layer was dried over anhydrous Na₂SO₄ and concentrated under reduced pressure to afford 5 g of crude compound. The material was purified by Combi-flash (silica gel) using 30-40% of ethyl acetate in petroleum ether as eluent to afford 3.1 g (57% yield) of methyl 2-((6-(1,3-dioxolan-2-yl)pyridin-2-yl)(hydroxy)methyl)acrylate. LCMS m/z found 265.91 [M+H]⁺, RT=1.21 min, (Method E).

Step iii. To the 3.1 g (11.6 mmol, 1 eq.) of methyl 2-((6-(1,3-dioxolan-2-yl)pyridin-2-yl)(hydroxy)methyl)acrylate was added Ac₂O (30 mL) and the mixture was heated to reflux for 16 h. After completion of reaction, the reaction mixture was evaporated to dryness. The obtained crude compound was mixed with another batch of same quantity and purified by Combi-flash (silica gel) using 0-40% of ethyl acetate in petroleum ether as eluent to afford 720 mg (24% yield) of methyl 5-(1,3-dioxolan-2-yl)indolizine-2-carboxylate as a pale yellow solid. LCMS m/z found 247.98 [M+H]⁺, RT=1.77 min, (Method E).

Step iv. To a stirred solution of 720 mg (2.9 mmol, 1 eq.) of methyl 5-(1,3-dioxolan-2-yl)indolizine-2-carboxylate in 10 mL of MeOH was slowly added dropwise 0.5 mL (11.6 mmol, 4 eq.) of formic acid at 0° C. and stirring was continued at room temperature for 16 h. After completion of the reaction, the mixture was poured slowly dropwise on ice and extracted with ethyl acetate (2×100 mL). Organic layer was separated, washed with water, dried over anhydrous Na₂SO₄ and concentrated under reduced pressure. Obtained crude compound was purified by column chromatography using silica gel (100-200 mesh) and 10% ethyl acetate in petroleum ether as eluent to afford 220 mg (37% yield) of methyl 5-formylindolizine-2-carboxylate, as a white solid. LCMS m/z found 203.95 [MH]⁺, RT=1.80 min, (Method E).

Step v. To a stirred solution of 220 mg (1.08 mmol, 1 eq.) of methyl 5-formylindolizine-2-carboxylate in 10 mL of DCM was slowly added 1.08 mL (3.25 mmol, 3 eq.) of DAST dropwise at 0° C. and stirring was continued for 16 h. After completion of the reaction, the mixture was poured slowly dropwise on ice and extracted with ethyl acetate (2×100 mL). Organic layer was separated, washed with water, dried over anhydrous Na₂SO₄ and concentrated under reduced pressure. Obtained crude compound was purified by column chromatography using silica gel (100-200 mesh) and 10% ethyl acetate in petroleum ether as eluent to afford 48 mg (20% yield) of methyl 5-(difluoromethyl)indolizine-2-carboxylate as a white solid. LCMS m/z found 225.94 [MH]⁺, (Method E).

Step vi. To a stirred solution of 48 mg (0.21 mmol, 1 eq.) of methyl 5-(difluoromethyl)indolizine-2-carboxylate in a mixture of THF:Water:MeOH (2:1:1) (3 mL), 28 mg (1.2 mmol, 2.5 eq.) of LiOH was added at 0° C. and the reaction was stirred at room temperature for 4 h. After completion of reaction, volatiles were evaporated and the mixture was acidified with 10% aq. KHSO₄ solution to pH ˜2. The precipitated solids were collected by filtration, washed with water and dried under vacuum to afford 35 mg (77% yield) of 5-(difluoromethyl)indolizine-2-carboxylic acid (VIdc) as a white solid. LCMS m/z found 211.92 [M+H]⁺, RT=1.44 min, (Method E).

### (S)-N-(8,9-Difluoro-6-oxo-1,4,5,6-tetrahydro-2H-pyrano[3,4-c]isoquinolin-1-yl)-5-(difluoromethyl)-N-methylindolizine-2-carboxamide (Compound 266)

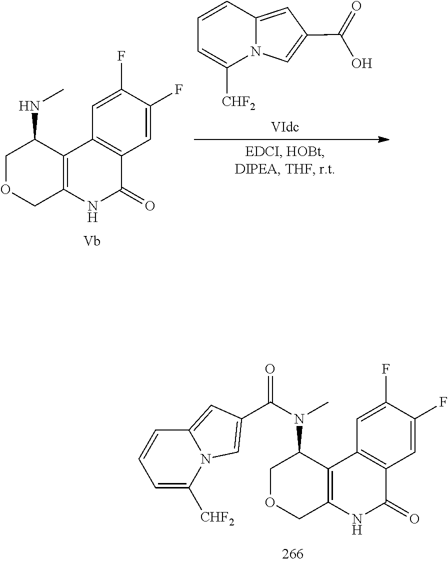

Enantiomerically pure (S)-N-(8,9-difluoro-6-oxo-1,4,5,6-tetrahydro-2H-pyrano[3,4-c]isoquinolin-1-yl)-5-(difluoromethyl)-N-methylindolizine-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5-(difluoromethyl)indolizine-2-carboxylic acid (VIdc). LCMS m/z found 460.3 [M+H]⁺; RT=7.68 min (Method A); ¹H NMR (400 MHz, DMSO-d₆) δ 11.70 (bs, 1H), 8.12 (t, 1H), 7.92 (s, 1H), 7.68 (d, 1H), 7.51-7.28 (m, 2H), 7.07 (d, 1H), 6.87 (t, 2H), 5.72 (s, 1H), 4.61 (d, 1H), 4.47 (d, 1H), 4.19 (d, 1H), 4.05-4.02 (m, 1H), 3.01 (s, 3H).

### (S)-N-(8,9-Difluoro-6-oxo-1,4,5,6-tetrahydro-2H-pyrano[3,4-c]isoquinolin-1-yl)-3′-fluoro-N-methyl-[1,1′-biphenyl]-4-carboxamide (Compound 283)

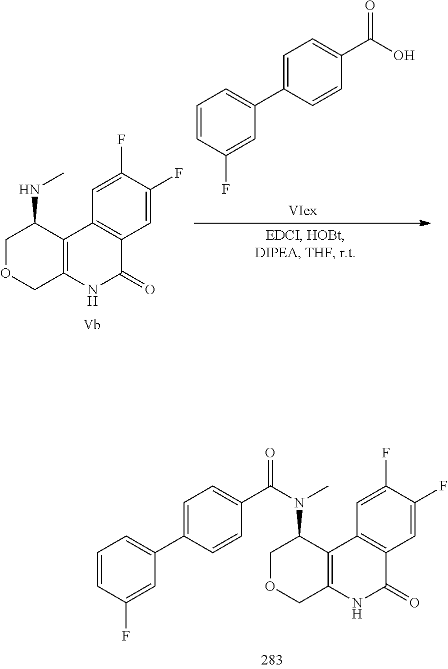

Enantiomerically pure (S)-N-(8,9-difluoro-6-oxo-1,4,5,6-tetrahydro-2H-pyrano[3,4-c]isoquinolin-1-yl)-3′-fluoro-N-methyl-[1,1′-biphenyl]-4-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3′-fluoro-[1,1′-biphenyl]-4-carboxylic acid (VIcx). LCMS m/z found 465.2 [M+H]⁺; RT=4.84 min (Method A); ¹H NMR (400 MHz, DMSO-d₆) δ 11.70 (bs, 1H), 8.14 (t, 1H), 7.80 (d, 2H), 7.57-7.49 (m, 6H), 7.23 (t, 1H), 5.71 (s, 1H), 4.60 (d, 1H), 4.48 (d, 1H), 4.29 (d, 1H), 4.07-4.03 (m, 1H), 2.78 (s, 3H).

### (S)-N-(8,9-Difluoro-6-oxo-1,4,5,6-tetrahydro-2H-pyrano[3,4-c]isoquinolin-1-yl)-2′-fluoro-N-methyl-[1,1′-biphenyl]-4-carboxamide (Compound 285)

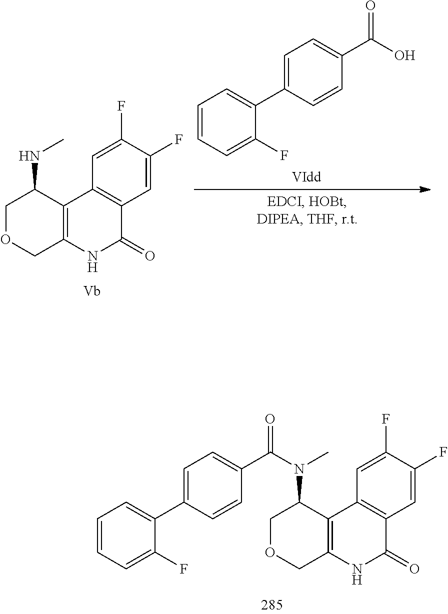

Enantiomerically pure (S)-N-(8,9-difluoro-6-oxo-1,4,5,6-tetrahydro-2H-pyrano[3,4-c]isoquinolin-1-yl)-2′-fluoro-N-methyl-[1,1′-biphenyl]-4-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 2′-fluoro-[1,1′-biphenyl]-4-carboxylic acid (VIdd). LCMS m/z found 465.2 [M+H]⁺; RT=4.75 min (Method A); ¹H NMR (400 MHz, DMSO-d₆) δ 11.70 (bs, 1H), 8.14 (t, 1H), 7.64 (d, 2H), 7.58-7.42 (m, 5H), 7.36-7.30 (m, 1H), 5.71 (s, 1H), 4.60 (d, 1H), 4.48 (d, 1H), 4.29 (d, 1H), 4.08-4.04 (m, 1H), 2.77 (s, 3H).

### (S)-N-(8,9-Difluoro-6-oxo-1,4,5,6-tetrahydro-2H-pyrano[3,4-c]isoquinolin-1-yl)-3′,5′-difluoro-N-methyl-[1,1′-biphenyl]-4-carboxamide (Compound 286)

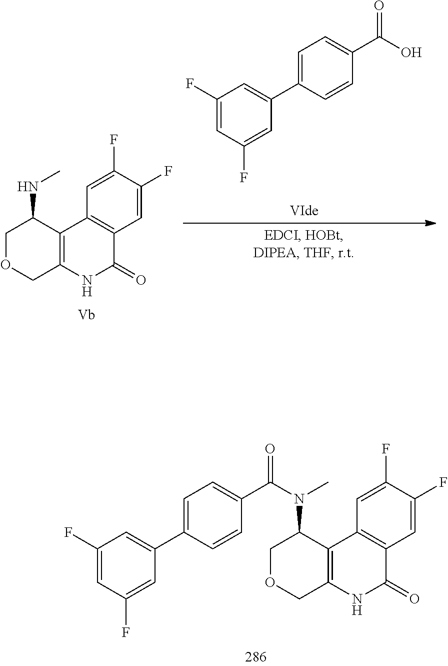 [SMILES: CN[C@@H]1COCc2[nH]c(=O)c3cc(F)c(F)cc3c21 ; O=C(O)c1ccc(-c2cc(F)cc(F)c2)cc1 ; CN(C(=O)c1ccc(-c2cc(F)cc(F)c2)cc1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-3′,5′-difluoro-N-methyl-[1,1′-biphenyl]-4-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3′,5′-difluoro-[1,1′-biphenyl]-4-carboxylic acid (VIde). LCMS m/z found 483.2 [M+H]⁺; RT=5.01 min (Method A); ¹H NMR (400 MHz, DMSO-d₆) δ 11.69 (bs, 1H), 8.14 (t, 1H), 7.84 (d, 2H), 7.54-7.49 (m, 5H), 7.29-7.24 (m, 1H), 5.71 (s, 1H), 4.60 (d, 1H), 4.47 (d, 1H), 4.29 (d, 1H), 4.08-4.03 (m, 1H), 2.75 (s, 3H).

### (S)-N-(8,9-Difluoro-6-oxo-1,4,5,6-tetrahydro-2H-pyrano[3,4-c]isoquinolin-1-yl)-2,3′,5′-trifluoro-N-methyl-[1,1′-biphenyl]-4-carboxamide (Compound 287)

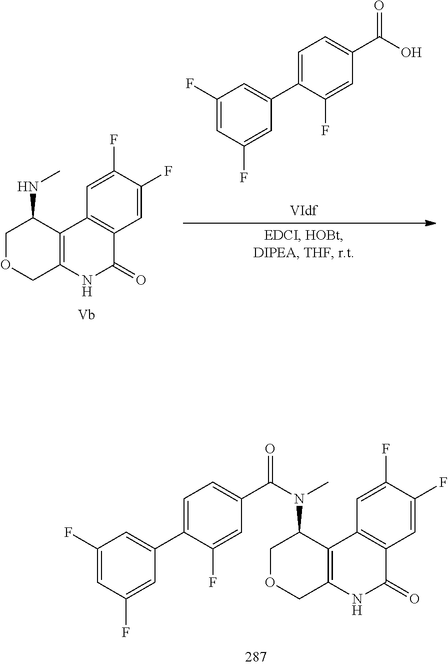 [SMILES: CN[C@@H]1COCc2[nH]c(=O)c3cc(F)c(F)cc3c21 ; O=C(O)c1ccc(-c2cc(F)cc(F)c2)c(F)c1 ; CN(C(=O)c1ccc(-c2cc(F)cc(F)c2)c(F)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-2,3′,5′-trifluoro-N-methyl-[1,1′-biphenyl]-4-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 2,3′,5′-trifluoro-[1,1′-biphenyl]-4-carboxylic acid (VIdf). LCMS m/z found 501.1 [M+H]⁺; RT=5.16 min (Method A); ¹H NMR (400 MHz, DMSO-d₆) δ 11.70 (bs, 1H), 8.14 (t, 1H), 7.69 (d, 1H), 7.52-7.45 (m, 2H), 7.37-7.32 (m, 4H), 5.69 (s, 1H), 4.60 (d, 1H), 4.47 (d, 1H), 4.30 (d, 1H), 4.06-4.02 (m, 1H), 2.76 (s, 3H).

### (S)-N-(8,9-Difluoro-6-oxo-1,4,5,6-tetrahydro-2H-pyrano[3,4-c]isoquinolin-1-yl)-N-methyl-[1,1′-biphenyl]-3-carboxamide (Compound 288)

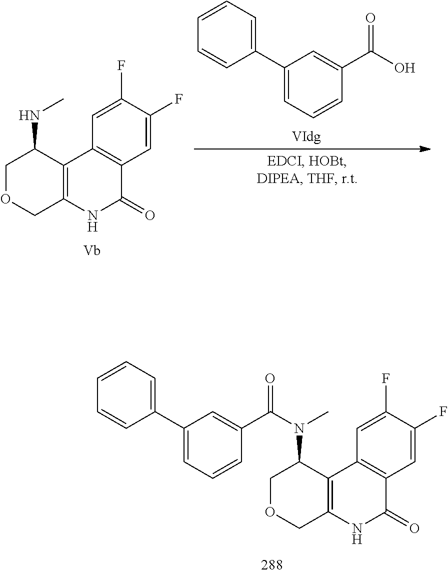 [SMILES: CN[C@@H]1COCc2[nH]c(=O)c3cc(F)c(F)cc3c21 ; O=C(O)c1cccc(-c2ccccc2)c1 ; CN(C(=O)c1cccc(-c2ccccc2)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-N-methyl-[1,1′-biphenyl]-3-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and [1,1′-biphenyl]-3-carboxylic acid (VIdg). LCMS m/z found 447.2 [M+H]⁺; RT=4.68 min (Method A); ¹H NMR (400 MHz, DMSO-d₆) δ 11.69 (bs, 1H), 8.14 (t, 1H), 7.75 (d, 1H), 7.68 (d, 3H), 7.57-7.47 (m, 4H), 7.41-7.35 (m, 2H), 5.73 (s, 1H), 4.59 (d, 1H), 4.47 (d, 1H), 4.31 (d, 1H), 4.07-4.03 (m, 1H), 2.75 (s, 3H).

### (S)-N-(8,9-Difluoro-6-oxo-1,4,5,6-tetrahydro-2H-pyrano[3,4-c]isoquinolin-1-yl)-4′-fluoro-N-methyl-[1,1′-biphenyl]-3-carboxamide (Compound 289)

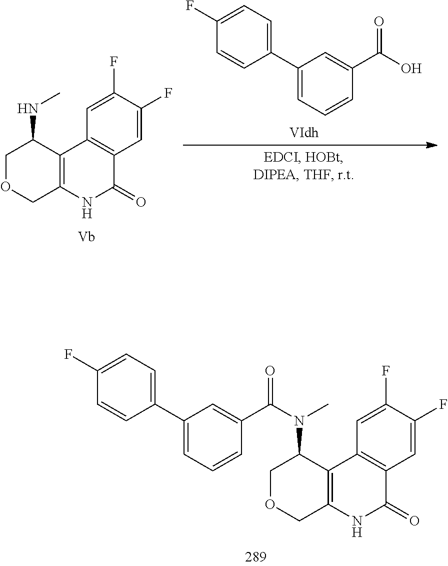 [SMILES: CN[C@@H]1COCc2[nH]c(=O)c3cc(F)c(F)cc3c21 ; O=C(O)c1cccc(-c2ccc(F)cc2)c1 ; CN(C(=O)c1cccc(-c2ccc(F)cc2)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-4′-fluoro-N-methyl-[1,1′-biphenyl]-3-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4′-fluoro-[1,1′-biphenyl]-3-carboxylic acid (VIdh). LCMS m/z found 465.2 [M+H]⁺; RT=4.75 min (Method A); ¹H NMR (400 MHz, DMSO-d₆) δ 11.69 (bs, 1H), 8.14 (t, 1H), 7.75-7.72 (m, 3H), 7.66 (s, 1H), 7.57-7.52 (m, 2H), 7.35-7.29 (m, 3H), 5.73 (s, 1H), 4.59 (d, 1H), 4.47 (d, 1H), 4.31 (d, 1H), 4.07-4.03 (m, 1H), 2.74 (s, 3H).

### (S)-N-(8,9-Difluoro-6-oxo-1,4,5,6-tetrahydro-2H-pyrano[3,4-c]isoquinolin-1-yl)-3′-fluoro-N-methyl-[1,1′-biphenyl]-3-carboxamide (Compound 290)

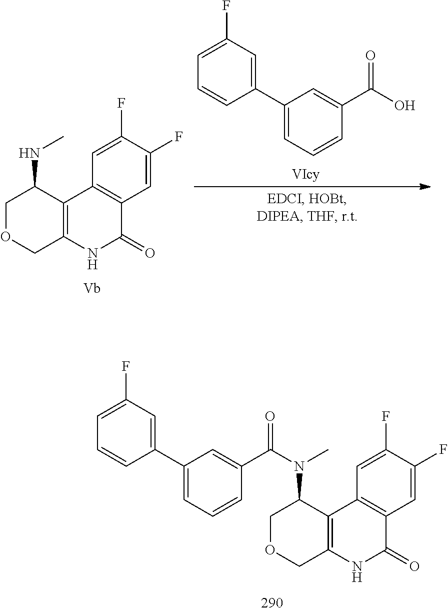 [SMILES: CN[C@@H]1COCc2[nH]c(=O)c3cc(F)c(F)cc3c21 ; O=C(O)c1cccc(-c2cccc(F)c2)c1 ; CN(C(=O)c1cccc(-c2cccc(F)c2)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-3′-fluoro-N-methyl-[1,1′-biphenyl]-3-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3′-fluoro-[1,1′-biphenyl]-3-carboxylic acid (VIcy). LCMS m/z found 465.2 [M+H]⁺; RT=4.75 min (Method A); ¹H NMR (400 MHz, DMSO-d₆) δ 11.69 (bs, 1H), 8.14 (t, 1H), 7.79 (s, 1H), 7.73 (s, 1H), 7.56-7.52 (m, 5H), 7.38 (d, 1H), 7.25-7.21 (m, 1H), 5.73 (s, 1H), 4.59 (d, 1H), 4.48 (d, 1H), 4.31 (d, 1H), 4.07-4.03 (m, 1H), 2.74 (s, 3H).

### (S)-N-(8,9-Difluoro-6-oxo-1,4,5,6-tetrahydro-2H-pyrano[3,4-c]isoquinolin-1-yl)-3′,5-difluoro-N-methyl-[1,1′-biphenyl]-3-carboxamide (Compound 291)

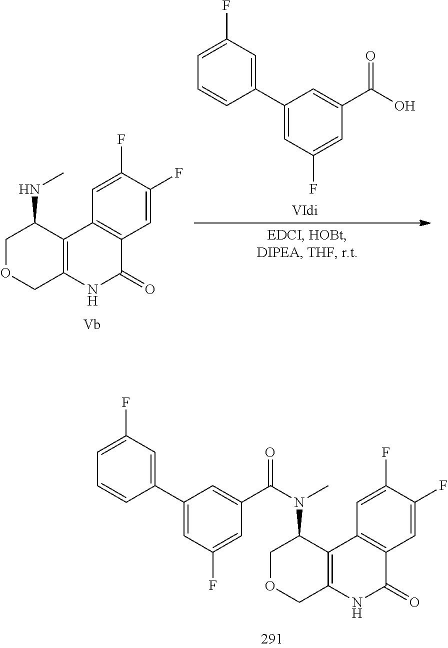 [SMILES: CN[C@@H]1COCc2[nH]c(=O)c3cc(F)c(F)cc3c21 ; O=C(O)c1cc(F)cc(-c2cccc(F)c2)c1 ; CN(C(=O)c1cc(F)cc(-c2cccc(F)c2)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-3′,5-difluoro-N-methyl-[1,1′-biphenyl]-3-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3′,5-difluoro-[1,1′-biphenyl]-3-carboxylic acid (VIdi). LCMS m/z found 483.2 [M+H]⁺; RT=4.96 min (Method A); ¹H NMR (400 MHz, DMSO-d₆) δ 11.67 (bs, 1H), 8.12 (t, 1H), 7.70 (s, 1H), 7.63-7.49 (m, 5H), 7.28-7.24 (m, 2H), 5.69 (s, 1H), 4.59 (d, 1H), 4.46 (d, 1H), 4.33 (d, 1H), 4.05-4.01 (m, 1H), 2.74 (s, 3H).

### (S)-N-(8,9-Difluoro-6-oxo-1,4,5,6-tetrahydro-2H-pyrano[3,4-c]isoquinolin-1-yl)-N-methyl-3-phenoxybenzamide (Compound 292)

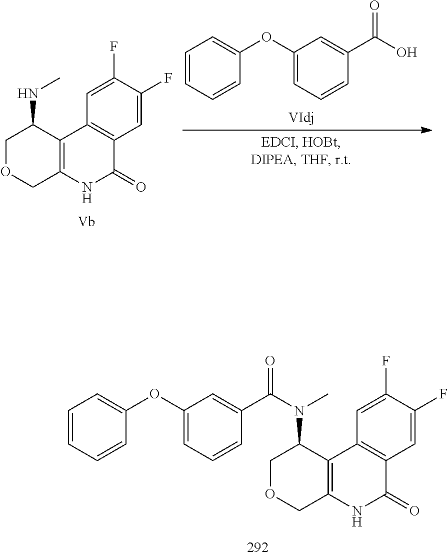

Enantiomerically pure (S)-N-(8,9-difluoro-6-oxo-1,4,5,6-tetrahydro-2H-pyrano[3,4-c]isoquinolin-1-yl)-N-methyl-3-phenoxybenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-phenoxybenzoic acid (VIdj). LCMS m/z found 463.2 [M+H]⁺; RT=4.68 min (Method A); ¹H NMR (400 MHz, DMSO-d₆) δ 11.67 (bs, 1H), 8.12 (t, 1H), 7.48-7.38 (m, 4H), 7.22-7.03 (m, 5H), 6.95 (s, 1H), 5.63 (s, 1H), 4.57 (d, 1H), 4.44 (d, 1H), 4.23 (d, 1H), 4.03-3.99 (m, 1H), 2.68 (s, 3H).

### (S)-N-(8,9-Difluoro-6-oxo-1,4,5,6-tetrahydro-2H-pyrano[3,4-c]isoquinolin-1-yl)-3-(4-fluorophenoxy)-N-methylbenzamide (Compound 293)

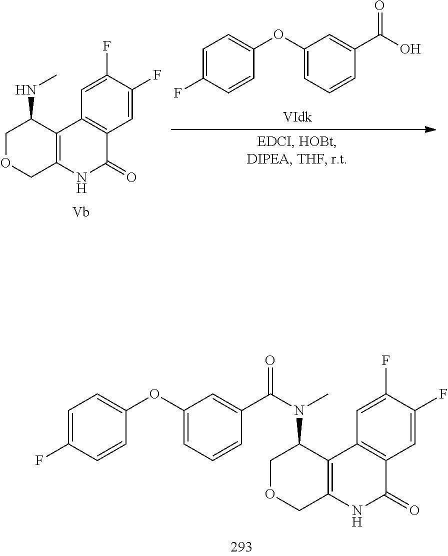

Enantiomerically pure (S)-N-(8,9-difluoro-6-oxo-1,4,5,6-tetrahydro-2H-pyrano[3,4-c]isoquinolin-1-yl)-3-(4-fluorophenoxy)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-(4-fluorophenoxy)benzoic acid (VIdk). LCMS m/z found 481.2 [M+H]⁺; RT=4.72 min (Method A); ¹H NMR (400 MHz, DMSO-d₆) δ 11.68 (bs, 1H), 8.12 (t, 1H), 7.46 (t, 1H), 7.40-7.35 (m, 1H), 7.23 (t, 2H), 7.12-7.06 (m, 4H), 6.90 (s, 1H), 5.63 (s, 1H), 4.57 (d, 1H), 4.45 (d, 1H), 4.23 (d, 1H), 4.03-3.99 (m, 1H), 2.67 (s, 3H).

### (S)-N-(8,9-Difluoro-6-oxo-1,4,5,6-tetrahydro-2H-pyrano[3,4-c]isoquinolin-1-yl)-N-methyl-4-(methylsulfonyl)benzamide (Compound 295)

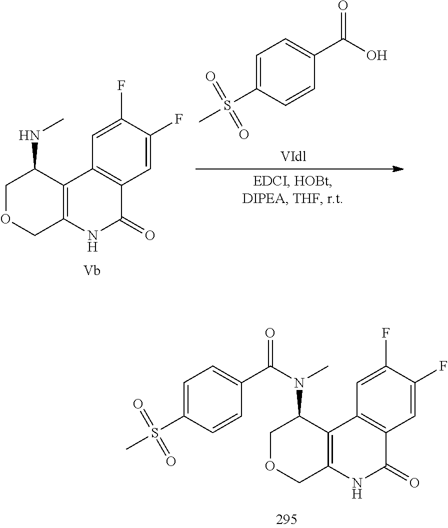

Enantiomerically pure (S)-N-(8,9-difluoro-6-oxo-1,4,5,6-tetrahydro-2H-pyrano[3,4-c]isoquinolin-1-yl)-N-methyl-4-(methylsulfonyl)benzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-(methylsulfonyl)benzoic acid (VIdl). LCMS m/z found 449.1 [M+H]⁺; RT=5.31 min (Method A); ¹H NMR (400 MHz, DMSO-d₆) δ 11.7 (bs, 1H), 8.14 (t, 1H), 8.01 (d, 2H), 7.67 (d, 2H), 7.51-7.46 (m, 1H), 5.7 (s, 1H), 4.59 (d, 1H), 4.47 (d, 1H), 4.31 (d, 1H), 4.06-4.02 (m, 1H), 3.26 (s, 3H), 2.69 (s, 3H).

### (S)-N-(8,9-Difluoro-6-oxo-1,4,5,6-tetrahydro-2H-pyrano[3,4-c]isoquinolin-1-yl)-N-methyl-3-(N-methylsulfamoyl)benzamide (Compound 296)

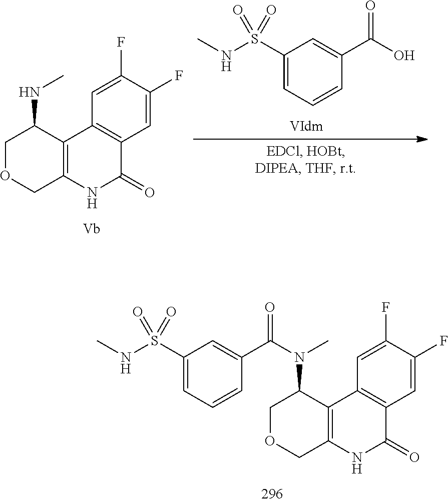

Enantiomerically pure (S)-N-(8,9-difluoro-6-oxo-1,4,5,6-tetrahydro-2H-pyrano[3,4-c]isoquinolin-1-yl)-N-methyl-3-(N-methylsulfamoyl)benzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-(N-methylsulfamoyl)benzoic acid (VIdm). LCMS m/z found 464.2 [M+H]⁺; RT=5.48 min (Method A); ¹H NMR (400 MHz, DMSO-d₆) δ 11.7 (s, 1H), 8.14 (t, 1H), 7.85 (d, 1H), 7.79 (s, 1H), 7.72-7.68 (m, 2H), 7.58 (d, 1H), 7.52-7.47 (m, 1H), 5.69 (s, 1H), 4.60 (d, 1H), 4.47 (d, 1H), 4.32 (d, 1H), 4.05-4.02 (m, 1H), 2.71 (s, 3H).), 2.42 (d, 3H).

### (S)-N-(8,9-Difluoro-6-oxo-1,4,5,6-tetrahydro-2H-pyrano[3,4-c]isoquinolin-1-yl)-N-methyl-3-(methylsulfonamido)benzamide (Compound 297)

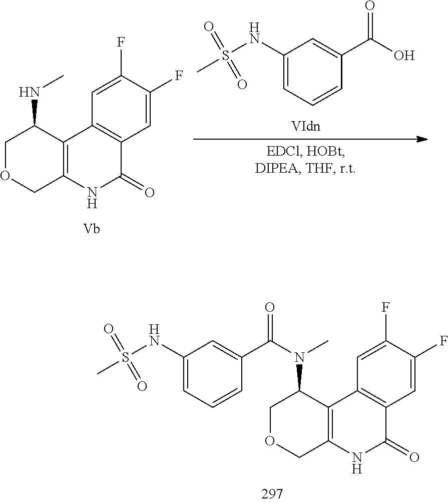

Enantiomerically pure (S)-N-(8,9-difluoro-6-oxo-1,4,5,6-tetrahydro-2H-pyrano[3,4-c]isoquinolin-1-yl)-N-methyl-3-(methylsulfonamido)benzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-(methylsulfonamido)benzoic acid (VIdn). LCMS m/z found 464.2 [M+H]⁺; RT=6.00 min (Method A); ¹H NMR (400 MHz, DMSO-d₆) δ 11.7 (s, 1H), 9.90 (bs, 1H), 8.16-8.11 (m, 1H), 7.52-7.39 (m, 2H), 7.34-7.27 (m, 1H), 7.19 (s, 1H), 7.11 (d, 1H), 5.66 (s, 1H), 4.59 (d, 1H), 4.46 (d, 1H), 4.23 (d, 1H), 4.06-4.02 (m, 1H), 3.0 (s, 3H), 2.70 (s, 3H).

### (S)-N-(8,9-Difluoro-6-oxo-1,4,5,6-tetrahydro-2H-pyrano[3,4-c]isoquinolin-1-yl)-N-methyl-3-(methylsulfonyl)benzamide (Compound 298)

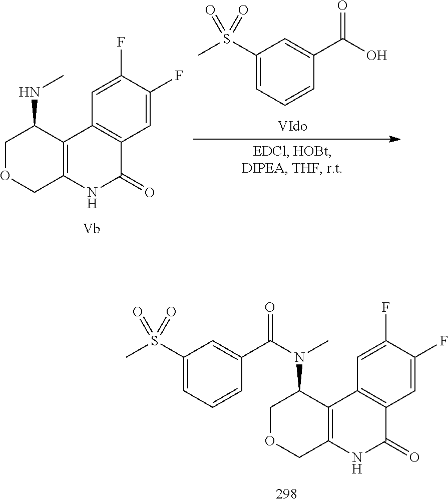

Enantiomerically pure (S)-N-(8,9-difluoro-6-oxo-1,4,5,6-tetrahydro-2H-pyrano[3,4-c]isoquinolin-1-yl)-N-methyl-3-(methylsulfonyl)benzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-(methylsulfonyl)benzoic acid (VIdo). LCMS m/z found 449.1 [M+H]⁺; RT=6.04 min (Method A); ¹H NMR (400 MHz, DMSO-d₆) δ 10.82 (bs, 1H), 8.11 (t, 1H), 8.01 (t, 1H), 7.96 (s, 1H), 7.74 (d, 2H), 7.5-7.45 (m, 1H), 5.69 (s, 1H), 4.54 (d, 1H), 4.45 (d, 1H), 4.33 (d, 1H), 4.04-4.01 (m, 1H), 3.27 (s, 3H), 2.70 (s, 3H).

### (S)-N-(8,9-Difluoro-6-oxo-1,4,5,6-tetrahydro-2H-pyrano[3,4-c]isoquinolin-1-yl)-3-fluoro-N-methyl-5-phenoxybenzamide (Compound 303)

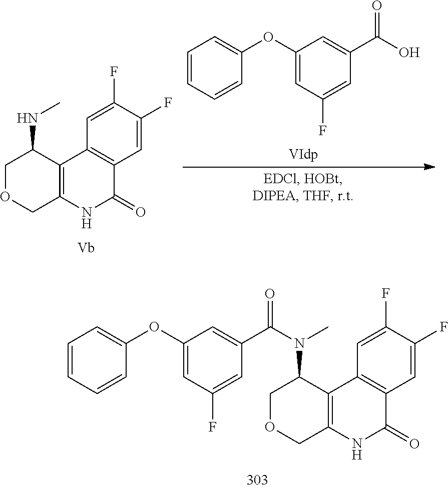

Enantiomerically pure (S)-N-(8,9-difluoro-6-oxo-1,4,5,6-tetrahydro-2H-pyrano[3,4-c]isoquinolin-1-yl)-3-fluoro-N-methyl-5-phenoxybenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-fluoro-5-phenoxybenzoic acid (VIdp). LCMS m/z found 481.2 [M+H]⁺; RT=4.93 min (Method A); ¹H NMR (400 MHz, DMSO-d₆) δ 11.67 (bs, 1H), 8.11 (m, 1H), 7.42 (t, 2H), 7.34 (m, 1H), 7.21 (t, 1H), 7.10 (m, 2H), 7.00 (m, 2H), 6.73 (s, 1H), 5.60 (s, 1H), 4.57 (d, 1H), 4.44 (d, 1H), 4.24 (d, 1H), 3.99 (m, 1H), 2.67 (s, 3H).

### (S)-N-(8,9-Difluoro-6-oxo-1,4,5,6-tetrahydro-2H-pyrano[3,4-c]isoquinolin-1-yl)-3-(3-fluorophenoxy)-N-methylbenzamide (Compound 322)

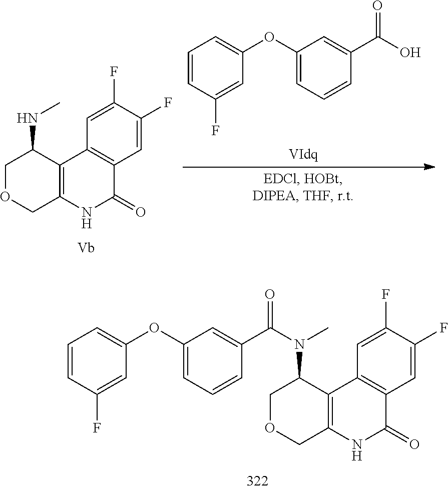

Enantiomerically pure (S)-N-(8,9-difluoro-6-oxo-1,4,5,6-tetrahydro-2H-pyrano[3,4-c]isoquinolin-1-yl)-3-(3-fluorophenoxy)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-(3-fluorophenoxy)benzoic acid (VIdq). LCMS m/z found 481.2 [M+H]⁺; RT=4.78 min (Method A); ¹H NMR (400 MHz, DMSO-d₆) δ 11.66 (bs, 1H), 8.08 (t, 1H), 7.51-7.34 (m, 3H), 7.16 (t, 2H), 7.03-6.85 (d, 4H), 5.62 (s, 1H), 4.54 (d, 1H), 4.42 (d, 1H), 4.23 (d, 1H), 4.0 (d, 1H), 2.68 (s, 3H).

### (S)-N-(8,9-Difluoro-6-oxo-1,4,5,6-tetrahydro-2H-pyrano[3,4-c]isoquinolin-1-yl)-5-methoxy-N-methyl-1H-indole-2-carboxamide (Compound 324)

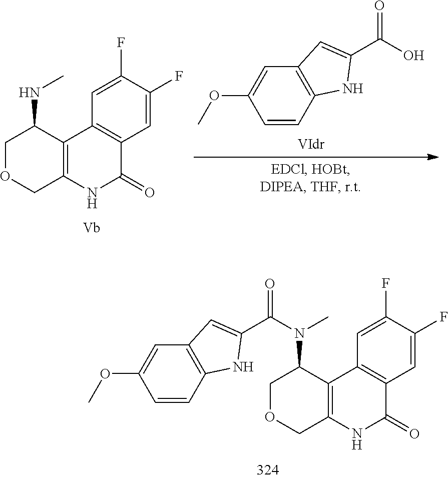

Enantiomerically pure (S)-N-(8,9-difluoro-6-oxo-1,4,5,6-tetrahydro-2H-pyrano[3,4-c]isoquinolin-1-yl)-5-methoxy-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5-methoxy-1H-indole-2-carboxylic acid (VIdr). LCMS m/z found 440.2 [M+H]⁺; RT=3.88 min (Method A); ¹H NMR (400 MHz, DMSO-d₆) δ 11.71 (bs, 1H), 11.57 (bs, 1H), 8.12 (t, 1H), 7.45-7.35 (m, 2H), 7.06 (s, 1H), 6.88-6.85 (m, 2H), 5.76 (s, 1H), 4.64 (d, 1H), 4.48 (d, 1H), 4.16 (d, 1H), 4.05 (d, 1H), 3.74 (s, 3H), 3.16 (s, 3H).

### (S)-5-chloro-N-(8,9-Difluoro-6-oxo-1,4,5,6-tetrahydro-2H-pyrano[3,4-c]isoquinolin-1-yl)-N-methyl-1H-indole-2-carboxamide (Compound 325)

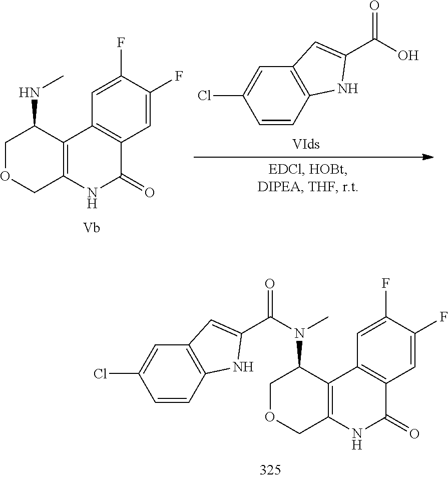

Enantiomerically pure (S)-5-chloro-N-(8,9-difluoro-6-oxo-1,4,5,6-tetrahydro-2H-pyrano[3,4-c]isoquinolin-1-yl)-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5-chloro-1H-indole-2-carboxylic acid (VIds). LCMS m/z found 444.2 [M+H]⁺; RT=4.61 min (Method A); ¹H NMR (400 MHz, DMSO-d₆) δ 11.92 (bs, 1H), 11.72 (bs, 1H), 8.13 (t, 1H), 7.66 (s, 1H), 7.49-7.45 (m, 2H), 7.22 (d, 1H), 6.93 (s, 1H), 5.75 (s, 1H), 4.64 (d, 1H), 4.48 (d, 1H), 4.17 (d, 1H), 4.04 (d, 1H), 3.15 (s, 3H).

### (S)-N-(8,9-Difluoro-6-oxo-1,4,5,6-tetrahydro-2H-pyrano[3,4-c]isoquinolin-1-yl)-N,5-dimethyl-1H-indole-2-carboxamide (Compound 326)

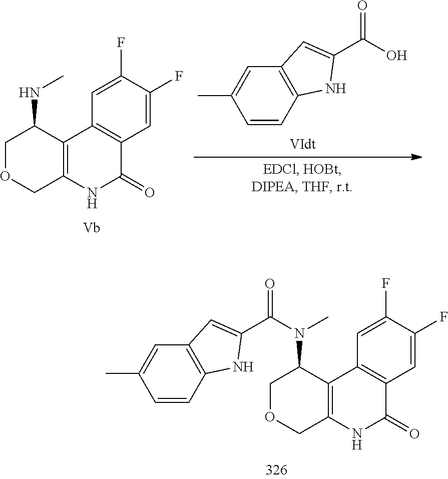

Enantiomerically pure (S)-N-(8,9-difluoro-6-oxo-1,4,5,6-tetrahydro-2H-pyrano[3,4-c]isoquinolin-1-yl)-N,5-dimethyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5-methyl-1H-indole-2-carboxylic acid (VIdt). LCMS m/z found 424.2 [M+H]⁺; RT=4.44 min (Method A); ¹H NMR (400 MHz, DMSO-d₆) δ 11.71 (bs, 1H), 11.58 (bs, 1H), 8.15-8.10 (m, 1H), 7.45-7.35 (m, 3H), 7.04 (d, 1H), 6.84 (s, 1H), 5.76 (s, 1H), 4.64 (d, 1H), 4.48 (d, 1H), 4.15 (d, 1H), 4.05 (d, 1H), 3.16 (s, 3H), 2.36 (s, 3H).

### (S)-N-(8,9-Difluoro-6-oxo-1,4,5,6-tetrahydro-2H-pyrano[3,4-c]isoquinolin-1-yl)-N-methyl-5-(trifluoromethoxy)-1H-indole-2-carboxamide (Compound 327)

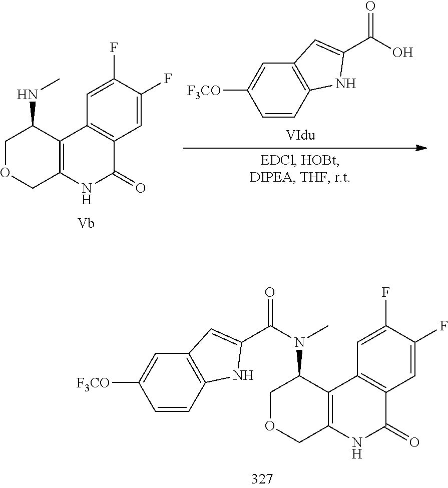 [SMILES: CN[C@@H]1COCc2[nH]c(=O)c3cc(F)c(F)cc3c21 ; O=C(O)c1cc2cc(OC(F)(F)F)ccc2[nH]1 ; CN(C(=O)c1cc2cc(OC(F)(F)F)ccc2[nH]1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-N-methyl-5-(trifluoromethoxy)-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5-(trifluoromethoxy)-1H-indole-2-carboxylic acid (VIdu). LCMS m/z found 494.2 [M+H]⁺; RT=4.92 min (Method A); ¹H NMR (400 MHz, DMSO-d₆) δ 12.0 (s, 1H), 11.72 (s, 1H), 8.13 (t, 1H), 7.60-7.54 (m, 2H), 7.46-7.41 (m, 1H), 7.20 (d, 1H), 7.01 (s, 1H), 5.75 (s, 1H), 4.66 (d, 1H), 4.49 (d, 1H), 4.19 (d, 1H), 4.05 (d, 1H), 3.16 (s, 3H).

### (S)-N-(8,9-Difluoro-6-oxo-1,4,5,6-tetrahydro-2H-pyrano[3,4-c]isoquinolin-1-yl)-5-ethyl-N-methyl-1H-indole-2-carboxamide (Compound 328)

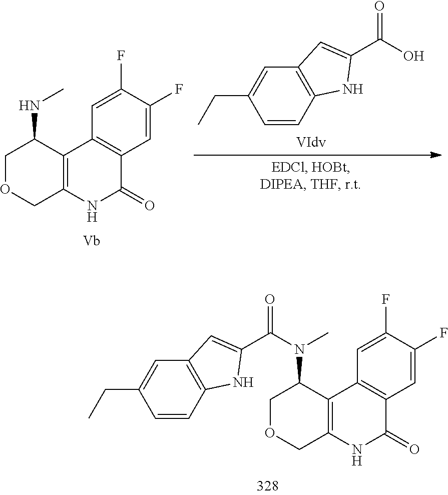

Enantiomerically pure (S)-N-(8,9-difluoro-6-oxo-1,4,5,6-tetrahydro-2H-pyrano[3,4-c]isoquinolin-1-yl)-5-ethyl-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5-ethyl-1H-indole-2-carboxylic acid (VIdv). LCMS m/z found 438.2 [M+H]⁺; RT=4.82 min (Method A); ¹H NMR (400 MHz, DMSO-d₆) δ 11.71 (s, 1H), 11.59 (s, 1H), 8.12 (t, 1H), 7.47-7.37 (m, 3H), 7.08 (d, 1H), 6.86 (s, 1H), 5.76 (s, 1H), 4.64 (d, 1H), 4.48 (d, 1H), 4.17 (d, 1H), 4.05 (d, 1H), 3.16 (s, 3H), 2.68-2.63 (m, 2H), 1.27-1.18 (t, 3H).

### (S)-N-(8,9-Difluoro-6-oxo-1,4,5,6-tetrahydro-2H-pyrano[3,4-c]isoquinolin-1-yl)-4-methoxy-N-methyl-1H-indole-2-carboxamide (Compound 329)

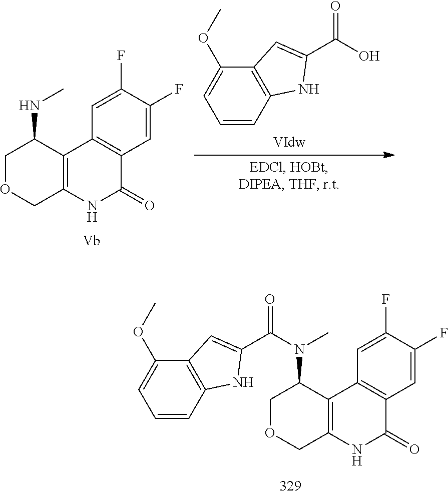

Enantiomerically pure (S)-N-(8,9-difluoro-6-oxo-1,4,5,6-tetrahydro-2H-pyrano[3,4-c]isoquinolin-1-yl)-4-methoxy-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-methoxy-1H-indole-2-carboxylic acid (VIdw). LCMS m/z found 440.2 [M+H]⁺; RT=4.00 min (Method A); ¹H NMR (400 MHz, DMSO-d₆) δ 11.73 (bs, 2H), 8.12 (t, 1H), 7.43 (bs, 1H), 7.15-7.05 (m, 2H), 6.88 (s, 1H), 6.52 (d, 1H), 5.75 (s, 1H), 4.64 (d, 1H), 4.48 (d, 1H), 4.15 (d, 1H), 4.05 (d, 1H), 3.85 (s, 3H), 3.17 (s, 3H).

### (S)-N-(8,9-Difluoro-6-oxo-1,4,5,6-tetrahydro-2H-pyrano[3,4-c]isoquinolin-1-yl)-N,4-dimethyl-1H-indole-2-carboxamide (Compound 330)

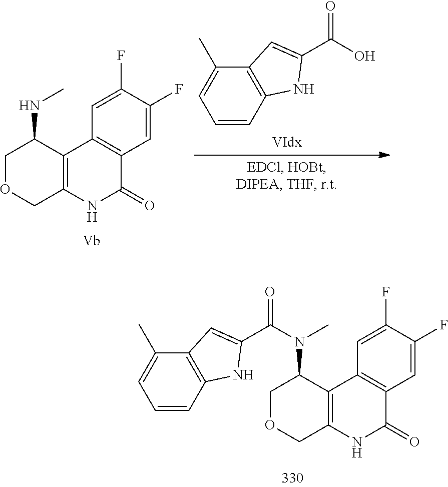

Enantiomerically pure (S)-N-(8,9-difluoro-6-oxo-1,4,5,6-tetrahydro-2H-pyrano[3,4-c]isoquinolin-1-yl)-N,4-dimethyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-methyl-1H-indole-2-carboxylic acid (VIdx). LCMS m/z found 424.2 [M+H]⁺; RT=4.36 min (Method A); ¹H NMR (400 MHz, DMSO-d₆) δ 11.72 (bs, 1H), 11.68 (bs, 1H), 8.13 (t, 1H), 7.47 (bs, 1H), 7.29 (d, 1H), 7.10 (t, 1H), 6.96 (s, 1H), 6.83 (d, 1H), 5.77 (s, 1H), 4.65 (d, 1H), 4.49 (d, 1H), 4.19-4.04 (m, 2H), 3.19 (s, 3H), 2.47 (s, 3H).

### (S)-4-Cyano-N-(8,9-difluoro-6-oxo-1,4,5,6-tetrahydro-2H-pyrano[3,4-c]isoquinolin-1-yl)-6-fluoro-N-methyl-1H-indole-2-carboxamide (Compound 341)

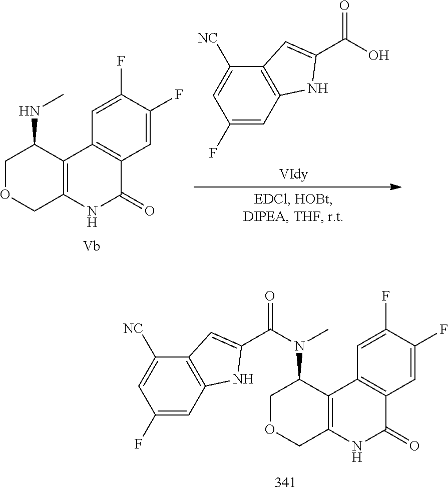

Enantiomerically pure (S)-4-cyano-N-(8,9-difluoro-6-oxo-1,4,5,6-tetrahydro-2H-pyrano[3,4-c]isoquinolin-1-yl)-6-fluoro-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-cyano-6-fluoro-1H-indole-2-carboxylic acid (VIdy). LCMS m/z found 453.2 [M+H]⁺; RT=4.10 min (Method A); ¹H NMR (400 MHz, DMSO-d₆) δ 11.92 (bs, 2H), 8.11 (t, 1H), 7.55-6.86 (m, 4H), 5.74 (s, 1H), 4.62 (d, 1H), 4.47 (d, 1H), 4.19 (d, 1H), 4.04 (d, 1H), 3.19 (s, 3H).

### (S)-N-(8,9-Difluoro-6-oxo-1,4,5,6-tetrahydro-2H-pyrano[3,4-c]isoquinolin-1-yl)-4-ethyl-6-fluoro-N-methyl-1H-indole-2-carboxamide (Compound 342)

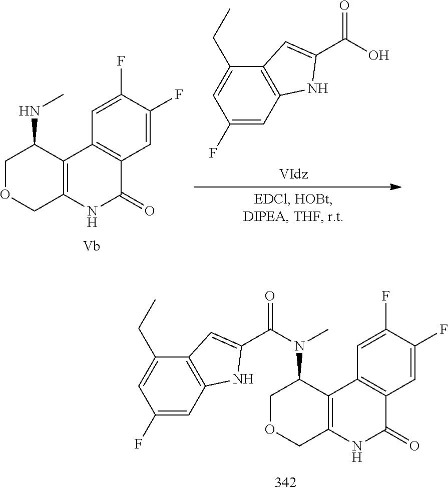

Enantiomerically pure (S)-N-(8,9-difluoro-6-oxo-1,4,5,6-tetrahydro-2H-pyrano[3,4-c]isoquinolin-1-yl)-4-ethyl-6-fluoro-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) 4-ethyl-6-fluoro-1H-indole-2-carboxylic acid (VIdz). LCMS m/z found 456.2 [M+H]⁺; RT=4.85 min (Method A); ¹H NMR (400 MHz, DMSO-d₆) δ 11.76 (bs, 2H), 8.12 (m, H1HH), 7.46 (bs, 1H), 7.01 (d, 2H), 6.76 (d, 1H), 5.76 (s, 1H), 4.64 (d, 1H), 4.48 (d, 1H), 4.17 (d, 1H), 4.04 (d, 1H), 3.18 (bs, 3H), 2.86 (d, 2H), 1.24 (t, 3H).

### (S)-4-Chloro-N-(8,9-difluoro-6-oxo-1,4,5,6-tetrahydro-2H-pyrano[3,4-c]isoquinolin-1-yl)-6-fluoro-N-methyl-1H-indole-2-carboxamide (Compound 343)

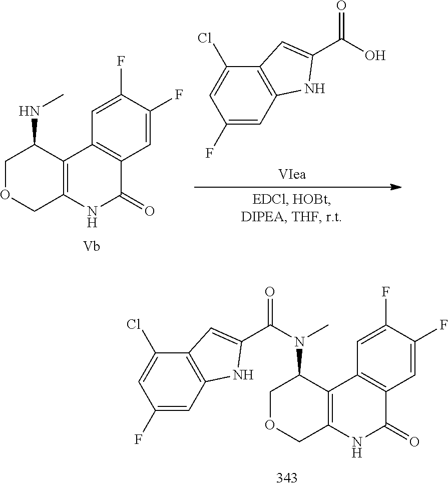

Enantiomerically pure (S)-4-chloro-N-(8,9-difluoro-6-oxo-1,4,5,6-tetrahydro-2H-pyrano[3,4-c]isoquinolin-1-yl)-6-fluoro-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-chloro-6-fluoro-1H-indole-2-carboxylic acid (VIea). LCMS m/z found 462.1 [M+H]⁺; RT=4.81 min (Method A); ¹H NMR (400 MHz, DMSO-d₆) δ 12.0 (bs, 2H), 8.10 (t, 1H), 7.48-7.36 (m, 1H), 7.21-7.14 (m, 2H), 6.97-6.92 (m, 1H), 5.74 (s, 1H), 4.62 (d, 1H), 4.46 (d, 1H), 4.17 (d, 1H), 4.03 (d, 1H), 3.17 (s, 3H).

### (S)-N-(8,9-Difluoro-6-oxo-1,4,5,6-tetrahydro-2H-pyrano[3,4-c]isoquinolin-1-yl)-6-fluoro-N,4-dimethyl-1H-indole-2-carboxamide (Compound 344)

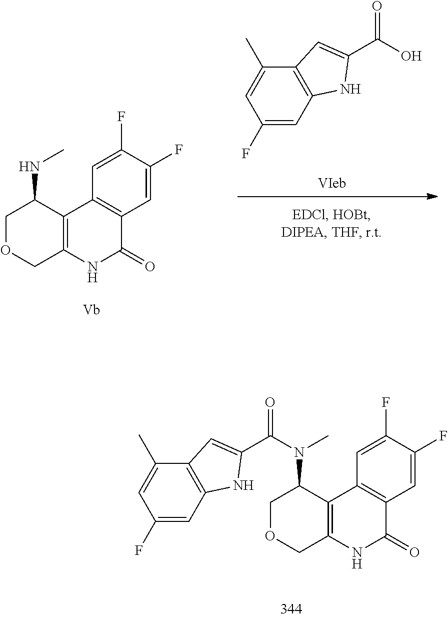 [SMILES: CN[C@@H]1COCc2[nH]c(=O)c3cc(F)c(F)cc3c21 ; Cc1cc(F)cc2[nH]c(C(=O)O)cc12 ; Cc1cc(F)cc2[nH]c(C(=O)N(C)[C@@H]3COCc4[nH]c(=O)c5cc(F)c(F)cc5c43)cc12]

Enantiomerically pure (S)-N-(8,9-difluoro-6-oxo-1,4,5,6-tetrahydro-2H-pyrano[3,4-c]isoquinolin-1-yl)-6-fluoro-N,4-dimethyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 6-fluoro-4-methyl-1H-indole-2-carboxylic acid (VIeb). LCMS m/z found 442.1 [M+H]⁺; RT=4.52 min (Method A); ¹H NMR (400 MHz, DMSO-d₆) δ 11.75 (bs, 2H), 8.12 (t, 1H), 7.45 (bs, 1H), 7.0 (bs, 2H), 6.74 (d, 1H), 5.76 (s, 1H), 4.65 (d, 1H), 4.48 (d, 1H), 4.16 (d, 1H), 4.05 (d, 1H), 3.18 (bs, 3H), 2.48 (s, 3H).

### (S)-5-Cyano-N-(8,9-difluoro-6-oxo-1,4,5,6-tetrahydro-2H-pyrano[3,4-c]isoquinolin-1-yl)-N-methyl-1H-indole-2-carboxamide (Compound 345)

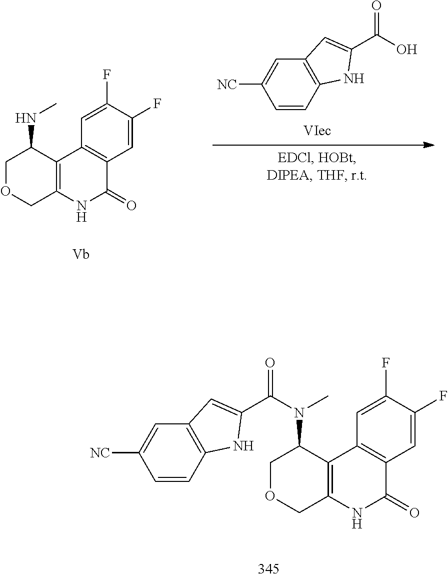 [SMILES: CN[C@@H]1COCc2[nH]c(=O)c3cc(F)c(F)cc3c21 ; N#Cc1ccc2[nH]c(C(=O)O)cc2c1 ; CN(C(=O)c1cc2cc(C#N)ccc2[nH]1)[C@@H]1COCc2[nH]c(=O)c3cc(F)c(F)cc3c21]

Enantiomerically pure (S)-5-cyano-N-(8,9-difluoro-6-oxo-1,4,5,6-tetrahydro-2H-pyrano[3,4-c]isoquinolin-1-yl)-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5-cyano-1H-indole-2-carboxylic acid (VIec). LCMS m/z found 435.3 [M+H]⁺; RT=3.69 min (Method A); ¹H NMR (400 MHz, DMSO-d₆) δ 12.29 (bs, 1H), 11.73 (bs, 1H), 8.18-8.10 (m, 2H), 7.63-7.54 (m, 2H), 7.46-7.41 (m, 1H), 7.09 (s, 1H), 5.74 (s, 1H), 4.68 (d, 1H), 4.52 (d, 1H), 4.20 (d, 1H), 4.08 (d, 1H), 3.16 (s, 3H).

### (S)-N-(8,9-Difluoro-6-oxo-1,4,5,6-tetrahydro-2H-pyrano[3,4-c]isoquinolin-1-yl)-N-methyl-5-(methylsulfonyl)-1H-indole-2-carboxamide (Compound 346)

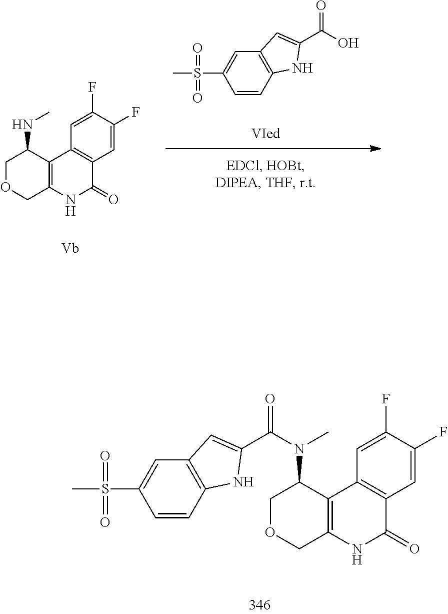 [SMILES: CN[C@@H]1COCc2[nH]c(=O)c3cc(F)c(F)cc3c21 ; CS(=O)(=O)c1ccc2[nH]c(C(=O)O)cc2c1 ; CN(C(=O)c1cc2cc(S(C)(=O)=O)ccc2[nH]1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-N-methyl-5-(methylsulfonyl)-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5-(methylsulfonyl)-1H-indole-2-carboxylic acid (VIed). LCMS m/z found 488.1 [M+H]⁺; RT=3.04 min (Method A); ¹H NMR (400 MHz, DMSO-d₆) δ 12.07 (bs, 2H), 8.23 (s, 1H), 8.10 (t, 1H), 7.73-7.65 (m, 2H), 7.42-7.38 (m, 1H), 7.18 (s, 1H), 5.75 (s, 1H), 4.62 (d, 1H), 4.47 (d, 1H), 4.18 (d, 1H), 4.05 (d, 1H), 3.16 (s, 6H).

### (S)-4-Chloro-N-(8,9-difluoro-6-oxo-1,4,5,6-tetrahydro-2H-pyrano[3,4-c]isoquinolin-1-yl)-N-methyl-1H-indole-2-carboxamide (Compound 347)

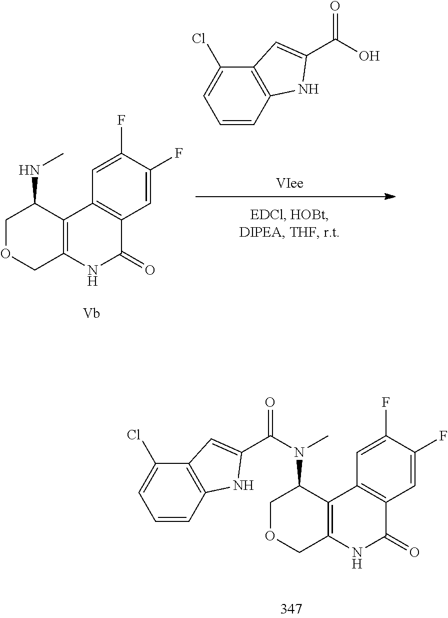 [SMILES: CN[C@@H]1COCc2[nH]c(=O)c3cc(F)c(F)cc3c21 ; O=C(O)c1cc2c(Cl)cccc2[nH]1 ; CN(C(=O)c1cc2c(Cl)cccc2[nH]1)[C@@H]1COCc2[nH]c(=O)c3cc(F)c(F)cc3c21]

Enantiomerically pure (S)-4-chloro-N-(8,9-difluoro-6-oxo-1,4,5,6-tetrahydro-2H-pyrano[3,4-c]isoquinolin-1-yl)-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-chloro-1H-indole-2-carboxylic acid (VIee). LCMS m/z found 444.1 [M+H]⁺; RT=4.56 min (Method A); ¹H NMR (400 MHz, DMSO-d₆) δ 12.10 (bs, 1H), 11.73 (bs, 1H), 8.13 (t, 1H), 7.45 (d, 2H), 7.21 (t, 1H), 7.13 (d, 1H), 6.90 (s, 1H), 5.76 (s, 1H), 4.64 (d, 1H), 4.49 (d, 1H), 4.20 (d, 1H), 4.05 (d, 1H), 3.18 (s, 3H).

### (S)-6-Bromo-N-(8,9-difluoro-6-oxo-1,4,5,6-tetrahydro-2H-pyrano[3,4-c]isoquinolin-1-yl)-N-methylindolizine-2-carboxamide (Compound 348)

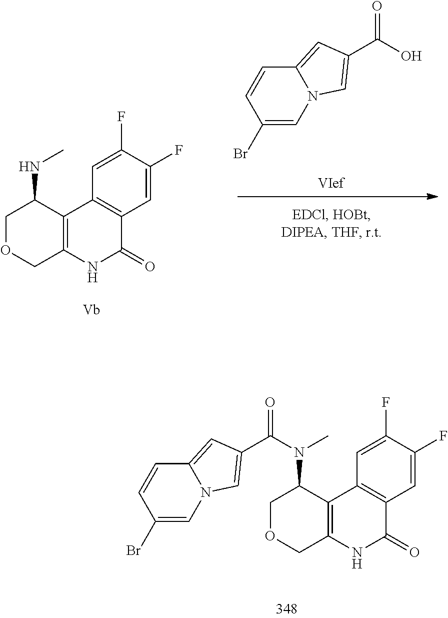 [SMILES: CN[C@@H]1COCc2[nH]c(=O)c3cc(F)c(F)cc3c21 ; O=C(O)c1cc2ccc(Br)cn2c1 ; CN(C(=O)c1cc2ccc(Br)cn2c1)[C@@H]1COCc2[nH]c(=O)c3cc(F)c(F)cc3c21]

Enantiomerically pure (S)-6-bromo-N-(8,9-difluoro-6-oxo-1,4,5,6-tetrahydro-2H-pyrano[3,4-c]isoquinolin-1-yl)-N-methylindolizine-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 6-bromoindolizine-2-carboxylic acid (VIef). LCMS m/z found 488.3 [M+H]⁺; ¹H NMR (400 MHz, DMSO-d₆) δ 11.69 (bs, 1H), 8.60 (s, 1H), 8.12 (t, 1H), 7.92 (s, 1H), 7.14 (d, 2H), 6.84 (d, 1H), 6.74 (s, 1H), 5.71 (s, 1H), 4.61 (d, 1H), 4.46 (d, 1H), 4.15 (d, 1H), 4.03 (d, 1H), 3.0 (s, 3H).

### 6-Fluoro-4-(hydroxymethyl)-1H-indole-2-carboxylic acid (VIeh)

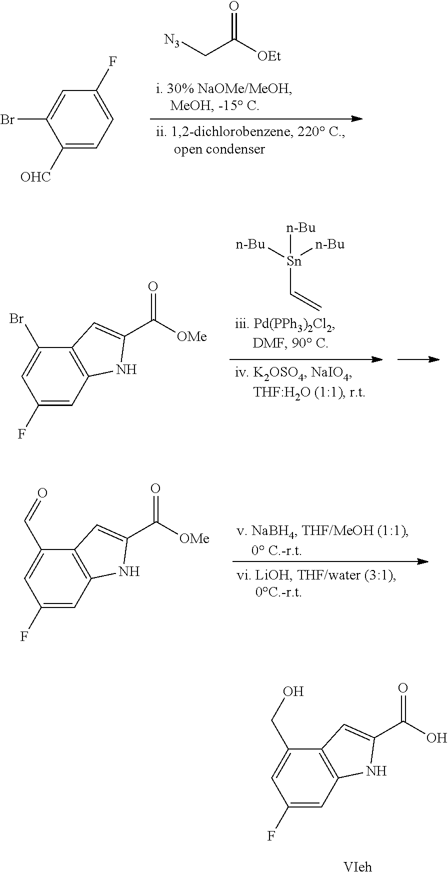

Step i. To a stirred solution 30% of NaOMe/MeOH in 15 mL of MeOH was added 5 g (24.3 mmol, 1 eq.) of 2-bromo-4-fluorobenzaldehyde and 7 g (54.2 mmol, 2.2 eq.) of ethyl 2-azidoacetate in 15 mL of MeOH was added dropwise at −15° C. The reaction mixture was stirred at same temperature for 3 h. After completion of the reaction (monitored by TLC), the mixture was poured dropwise on stirred ice and stirring was continued for an additional 30 min. The precipitated solids were collected by filtration, washed with water, dried, and purified by column chromatography (silica gel 100-200 mesh and 10% ethyl acetate/petroleum ether) to afford 2 g (27% yield) of methyl (Z)-2-azido-3-(2-bromo-4-fluorophenyl)acrylate. ¹H NMR (400 MHz, DMSO-d₆) δ 8.22-8.18 (m, 1H), 7.73-7.70 (m, 1H), 7.38-7.33 (m, 1H), 7.04 (s, 1H), 3.88 (s, 3H).

Step ii. To a solution of 2 g (29.8 mmol, 1 eq.) of methyl (Z)-2-azido-3-(2-bromo-4-fluorophenyl)acrylate in 20 mL of 1,2-dichlorobenzene was heated at 220° C., using a heating mantle with open condenser, for 1 h. After completion of the reaction (monitored by TLC), the mixture was diluted with petroleum ether (30 mL) and stirred at 0° C. for 10 min. The precipitated solid was collected by filtration, washed petroleum ether (20 mL) and dried under vacuum to afford 900 mg (49% yield) of methyl 4-bromo-6-fluoro-1H-indole-2-carboxylate as an off white solid. LCMS m/z found 271.84 [M+H]⁺, RT: 2.09 min (Method E).

Step iii. To a stirred solution of 500 mg (1.83 mmol, 1 eq.) of methyl 4-bromo-6-fluoro-1H-indole-2-carboxylate in 5 mL of DMF (10 vol) were added 0.65 mL (2.2 mmol, 1.2 eq.) of tri-butyl vinyltin. The resulting mixture was degassed with argon for 15 min. Pd(PPh₃)₂Cl₂ (130 mg, 0.18 mmol, 0.1 eq.) was added at room temperature and the reaction mixture was stirred at 90° C. for 16 h. After completion of the reaction (monitored by TLC), the mixture was filtered through a pad of CELITE® and the filtrate was diluted with ice-water (100 mL) and extracted with ethyl acetate (2×100 mL). Combined organic layer was washed with ice-water (50 mL), dried over anhydrous Na₂SO₄, filtered and concentrated under reduced pressure. Obtained compound was purified by column chromatography (silica gel 100-200 mesh and 10% ethyl acetate/petroleum ether) to afford 300 mg (74% yield) of methyl 6-fluoro-4-vinyl-1H-indole-2-carboxylate, as off white solid. LCMS m/z found 220.24 [M+H]⁺, RT=2.07 min (Method E).

Step iv. To a stirred solution of 300 mg (1.36 mmol, 1 eq.) of methyl 6-fluoro-4-vinyl-1H-indole-2-carboxylate in 3 mL of THF and 3 mL of water were added 5 mg (0.013 mmol, 0.01 eq.) of K₂OSO₄ and 670 mg (3.18 mmol, 2.3 eq.) of NaIO₄ at 0° C., and the reaction mixture was stirred at room temperature for 16 h. After completion of the reaction (monitored by TLC), the mixture was filtered through a pad of CELITE® and the filtrate was diluted with water (30 mL) and extracted with ethyl acetate (2×100 mL). Combined organic layer was washed with water (30 mL), dried over anhydrous Na₂SO₄, filtered and concentrated under reduced pressure. Obtained compound was purified by column chromatography (silica gel 100-200 mesh and 10% ethyl acetate/petroleum ether) to afford 150 mg (49% yield) of methyl 6-fluoro-4-formyl-1H-indole-2-carboxylate as an off-white solid. LCMS m/z found 222.20 [M+H]⁺, RT=1.72 min (Method E).

Step v. To a stirred solution of 240 mg (0.9 mmol, 1 eq.) of methyl 6-fluoro-4-formyl-1H-indole-2-carboxylate in 3 mL of methanol at room temperature under inert atmosphere, 51 mg (1.35 mmol, 1.5 eq.) of NaBH₄ was added portion-wise at 0° C. and the reaction mixture was stirred at room temperature for 4 h. After completion of reaction (monitored by TLC and LCMS analysis), the mixture was diluted with water (50 mL), filtered and the collected solids were washed with ethyl acetate (50 ml). The organic layer was separated, and the aqueous layer was extracted with ethyl acetate (3×50 mL). The combined organic extracts were dried over anhydrous Na₂SO₄, filtered, and concentrated under reduced pressure. The resulting product was triturated with diethyl ether (10 mL) and dried under vacuum to afford 200 mg of methyl 6-fluoro-4-(hydroxymethyl)-1H-indole-2-carboxylate as a light brown solid, which was carried into the next step without further purification. LCMS m/z found 238.21 [M+H]⁺.

Step vi. To a stirred solution of 180 mg (0.8 mmol, 1 eq.) of methyl 6-fluoro-4-(hydroxymethyl)-1H-indole-2-carboxylate in a mixture of THF:Water:MeOH (2:1:1) (5 mL), 169 mg (2.53 mmol, 3 eq.) of LiOH was added at 0° C. and the reaction was stirred at room temperature for 4 h. After completion of reaction (monitored by TLC), volatiles were evaporated and the residue was acidified with 10% aq. KHSO₄ solution to pH ˜2. The precipitated solids were collected by filtration, washed with water, and dried under vacuum to afford 130 mg (77% yield) of 6-fluoro-4-(hydroxymethyl)-1H-indole-2-carboxylic acid (VIeh), as a white solid. LCMS m/z found 222.16 [M−H]⁻, RT=1.39 min, (Method E).

### (S)-N-(8,9-Difluoro-6-oxo-1,4,5,6-tetrahydro-2H-pyrano[3,4-c]isoquinolin-1-yl)-6-fluoro-4-(hydroxymethyl)-N-methyl-1H-indole-2-carboxamide (Compound 355)

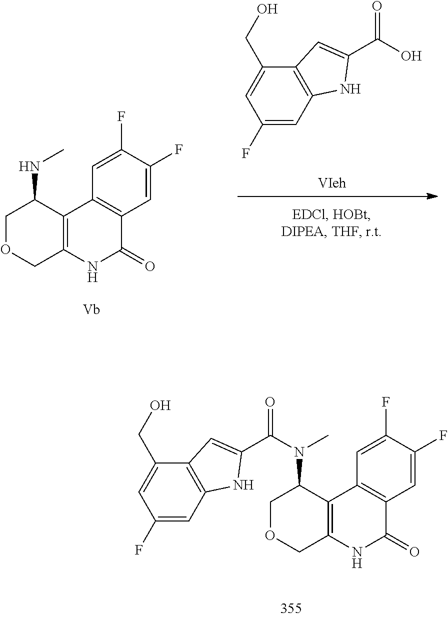 [SMILES: CN[C@@H]1COCc2[nH]c(=O)c3cc(F)c(F)cc3c21 ; O=C(O)c1cc2c(CO)cc(F)cc2[nH]1 ; CN(C(=O)c1cc2c(CO)cc(F)cc2[nH]1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-6-fluoro-4-(hydroxymethyl)-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 6-fluoro-4-(hydroxymethyl)-1H-indole-2-carboxylic acid (VIeh). LCMS m/z found 458.2 [M+H]⁺; RT=3.29 min (Method A); ¹H NMR (400 MHz, DMSO-d₆) δ 11.79 (bs, 1H), 11.71 (bs, 1H), 8.12 (t, 1H), 7.44 (bs, 1H), 7.04 (t, 2H), 6.92 (d, 1H), 5.75 (s, 1H), 5.30 (t, 1H), 4.76 (bs, 2H), 4.64 (d, 1H), 4.48 (d, 1H), 4.16 (d, 1H), 4.04 (d, 1H), 3.16 (s, 3H).

### 6-fluoro-4-(methylsulfonamido)-1H-indole-2-carboxylic acid (VIei)

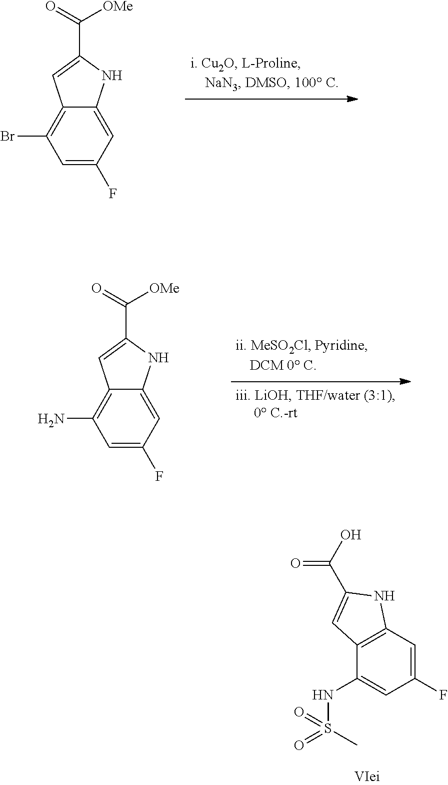 [SMILES: COC(=O)c1cc2c(Br)cc(F)cc2[nH]1 ; COC(=O)c1cc2c(N)cc(F)cc2[nH]1 ; CS(=O)(=O)Nc1cc(F)cc2[nH]c(C(=O)O)cc12]

Step i. To a stirred solution of 500 mg (1.83 mmol, 1 eq.) of methyl 4-bromo-6-fluoro-1H-indole-2-carboxylate in 5 mL of DMSO at room temperature under inert atmosphere, 52 mg (0.36 mmol, 0.2 eq.) of Cu₂O, 347 mg (5.51 mmol, 3 eq.) of NaN₃, and 105 mg (0.36 mmol, 0.2 eq.) of L-proline were added and the mixture was stirred at 100° C. for 16 h. After completion of reaction (monitored by TLC and LCMS analysis), the mixture was diluted with water (50 mL), filtered, and the heterogeneous mixture was extracted with ethyl acetate (50 mL). The organic layer was separated, and the aqueous layer was extracted with ethyl acetate (3×50 mL). The combined organic layer was dried over anhydrous Na₂SO₄, filtered and concentrated under reduced pressure. The resulting product was triturated with diethyl ether (10 mL), filtered and dried under vacuum to afford 200 mg of methyl 4-amino-6-fluoro-1H-indole-2-carboxylate as a light brown solid, which was carried into the next step without further purification. LCMS m/z found 207.12 [M−H]⁻, RT=1.32 min (Method E).

Step ii. To a solution of 400 mg (1.48 mmol, 1 eq.) of 4-amino-6-fluoro-1H-indole-2-carboxylate in 3 mL of DCM, 0.13 mL (4.46 mmol, 3 eq.) of Mesyl chloride and Pyridine (0.17 mL, 1.5 eq.) were added, and the reaction mixture was stirred at room temperature for 4 h. After completion of reaction (monitored by TLC), the mixture was cooled to room temperature and poured into ice cold water (20 mL) then extracted with EtOAC (2×50 mL). The combined organic layer was washed with brine (50 mL), dried over anhydrous Na₂SO₄, filtered and concentrated under reduced pressure. The resulting crude product was triturated with diethyl ether to afford 320 mg (77% yield) of methyl 6-fluoro-4-(methylsulfonamido)-1H-indole-2-carboxylate. LCMS m/z found 285.17 [M−H]⁻, RT=1.41 min (Method E).

Step iii. Methyl 6-fluoro-4-(methylsulfonamido)-1H-indole-2-carboxylate obtained in Step ii was converted to 6-fluoro-4-(methylsulfonamido)-1H-indole-2-carboxylic acid (VIei) in a similar manner as described above for VIeh. LCMS m/z found 271.23 [M−H]⁻, RT=1.13 min (Method E).

### (S)-N-(8,9-Difluoro-6-oxo-1,4,5,6-tetrahydro-2H-pyrano[3,4-c]isoquinolin-1-yl)-6-fluoro-N-methyl-4-(methylsulfonamido)-1H-indole-2-carboxamide (Compound 362)

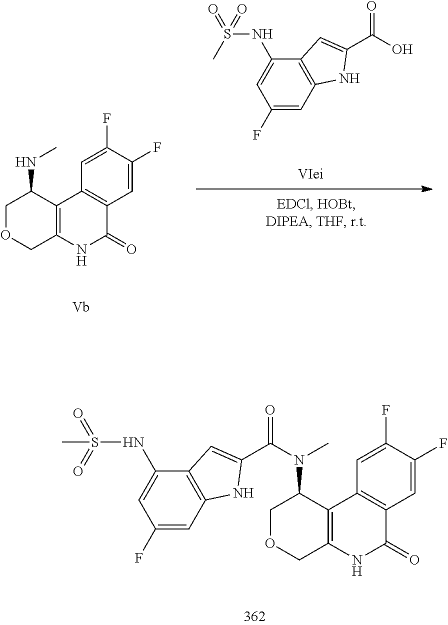 [SMILES: CN[C@@H]1COCc2[nH]c(=O)c3cc(F)c(F)cc3c21 ; CS(=O)(=O)Nc1cc(F)cc2[nH]c(C(=O)O)cc12 ; CN(C(=O)c1cc2c(NS(C)(=O)=O)cc(F)cc2[nH]1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-6-fluoro-N-methyl-4-(methylsulfonamido)-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 6-fluoro-4-(methylsulfonamido)-1H-indole-2-carboxylic acid (VIei). LCMS m/z found 521.2 [M+H]⁺; RT=3.54 min (Method A); ¹H NMR (400 MHz, DMSO-d₆) δ 11.89 (bs, 1H), 11.73 (bs, 1H), 9.98 (bs, 1H), 8.13 (t, 1H), 7.46 (bs, 2H), 6.94-6.87 (m, 2H), 5.76 (s, 1H), 4.65 (d, 1H), 4.49 (d, 1H), 4.17-4.03 (m, 2H), 3.17 (s, 3H), 3.05 (s, 3H).

### (S)-N-(8,9-Difluoro-6-oxo-1,4,5,6-tetrahydro-2H-pyrano[3,4-c]isoquinolin-1-yl)-N-methyl-4-(trifluoromethoxy)-1H-indole-2-carboxamide (Compound 363)

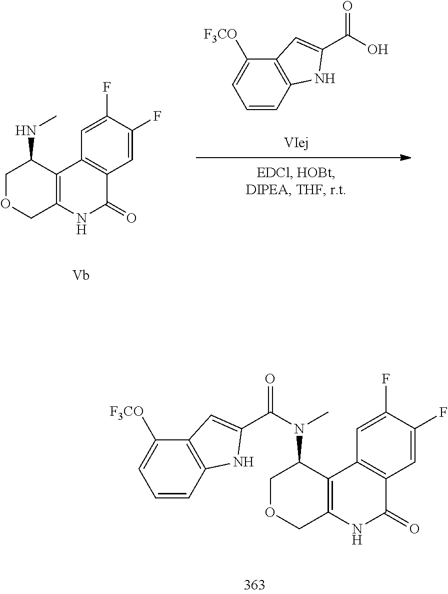 [SMILES: CN[C@@H]1COCc2[nH]c(=O)c3cc(F)c(F)cc3c21 ; O=C(O)c1cc2c(OC(F)(F)F)cccc2[nH]1 ; CN(C(=O)c1cc2c(OC(F)(F)F)cccc2[nH]1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-N-methyl-4-(trifluoromethoxy)-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-(trifluoromethoxy)-1H-indole-2-carboxylic acid (VIej). LCMS m/z found 494.2 [M+H]⁺; RT=4.84 min (Method A); ¹H NMR (400 MHz, DMSO-d₆) δ 12.17 (bs, 1H), 11.72 (s, 1H), 8.13 (t, 1H), 7.51-7.43 (m, 2H), 7.28 (t, 1H), 7.04 (d, 1H), 6.94 (bs, 1H), 5.75 (s, 1H), 4.64 (d, 1H), 4.49 (d, 1H), 4.20 (d, 1H), 4.05 (d, 1H), 3.16 (s, 3H).

### (S)-N-(8,9-Difluoro-6-oxo-1,4,5,6-tetrahydro-2H-pyrano[3,4-c]isoquinolin-1-yl)-6-methoxy-N-methyl-1H-indole-2-carboxamide (Compound 364)

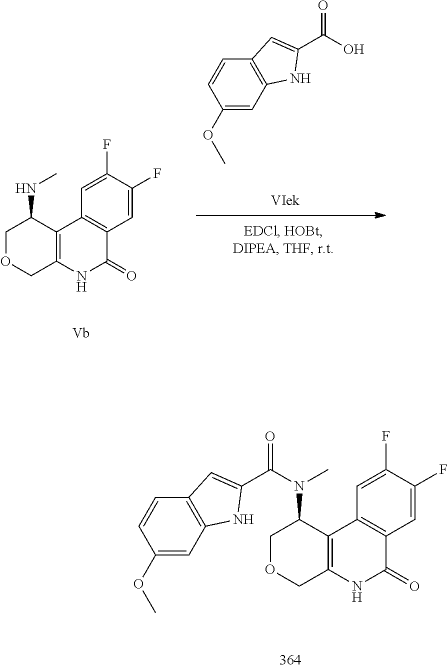 [SMILES: CN[C@@H]1COCc2[nH]c(=O)c3cc(F)c(F)cc3c21 ; COc1ccc2cc(C(=O)O)[nH]c2c1 ; COc1ccc2cc(C(=O)N(C)[C@@H]3COCc4[nH]c(=O)c5cc(F)c(F)cc5c43)[nH]c2c1]

Enantiomerically pure (S)-N-(8,9-difluoro-6-oxo-1,4,5,6-tetrahydro-2H-pyrano[3,4-c]isoquinolin-1-yl)-6-methoxy-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 6-methoxy-1H-indole-2-carboxylic acid (VIek). LCMS m/z found 440.2 [M+H]⁺; RT=3.98 min (Method A); ¹H NMR (400 MHz, DMSO-d₆) δ 11.71 (bs, 1H), 11.53 (bs, 1H), 8.12 (t, 1H), 7.48 (d, 2H), 6.91 (d, 2H), 6.70 (dd, 1H), 5.76 (bs, 1H), 4.64 (d, 1H), 4.48 (d, 1H), 4.14 (d, 1H), 4.04 (d, 1H), 3.78 (s, 3H), 3.16 (s, 3H).

### 6-(Difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide (Compounds 217 and 218)

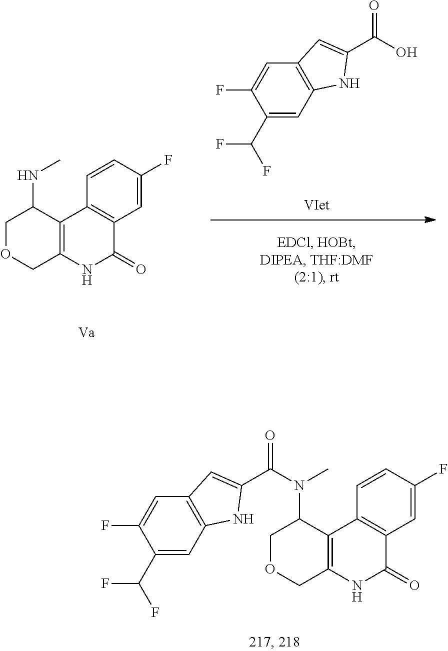 [SMILES: CNC1COCc2[nH]c(=O)c3cc(F)ccc3c21 ; O=C(O)c1cc2cc(F)c(C(F)F)cc2[nH]1 ; CN(C(=O)c1cc2cc(F)c(C(F)F)cc2[nH]1)C1COCc2[nH]c(=O)c3cc(F)ccc3c21]

Racemic 6-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from racemic 8-fluoro-1-(methylamino)-1,5-dihydro-2H-pyrano[3,4-c]isoquinolin-6(4H)-one (Va) and 6-(difluoromethyl)-5-fluoro-1H-indole-2-carboxylic acid (VIct). The enantiomers were subsequently separated by preparative SFC: Method isocratic, Mobile phase MeOH:CO₂—25:75. Column: Chiralcel-IC (30×250 mm), 5 μm, flow rate: 100 g/min.

Enantiomer I (Compound 217): LCMS: m/z found 460.3 [M+H]⁺, RT=5.20 min, (Method A); ¹H NMR (400 MHz, DMSO-d₆) δ 12.11 (s, 1H), 11.7 (s, 1H), 7.9-7.8 (m, 1H), 7.79-7.64 (d, 2H), 7.59-7.51 (m, 2H), 7.2 (s, 1H), 6.9 (s, 1H), 5.75 (s, 1H), 4.65 (d, 1H), 4.54 (d, 1H), 4.16 (d, 1H), 4.04 (dd, 1H), 3.12 (s, 3H); Chiral analytical SFC: RT=3.40 min, Column: Chiralpak IC-3 (4.6×150 mm) 3 μm, 30% MeOH, Flow rate: 3.0 g/min.

Enantiomer II (Compound 218): LCMS: m/z found 460.3 [M+H]⁺, RT=5.20 min, (Method A); ¹H NMR (400 MHz, DMSO-d₆) δ 12.11 (s, 1H), 11.7 (s, 1H), 7.9-7.8 (m, 1H), 7.79-7.64 (d, 2H), 7.59-7.51 (m, 2H), 7.2 (s, 1H), 6.9 (s, 1H), 5.75 (s, 1H), 4.65 (d, 1H), 4.54 (d, 1H), 4.16 (d, 1H), 4.04 (dd, 1H), 3.12 (s, 3H); Chiral analytical SFC: RT=4.32 min, Column: Chiralpak IC-3 (4.6×150 mm) 3 μm, 30% MeOH, Flow rate: 3.0 g/min.

### 4-(Difluoromethyl)-6-fluoro-N-(8-fluoro-6-oxo-1,4,5,6-tetrahydro-2H-pyrano[3,4-c]isoquinolin-1-yl)-N-methyl-1H-indole-2-carboxamide (Compounds 238 and 239)

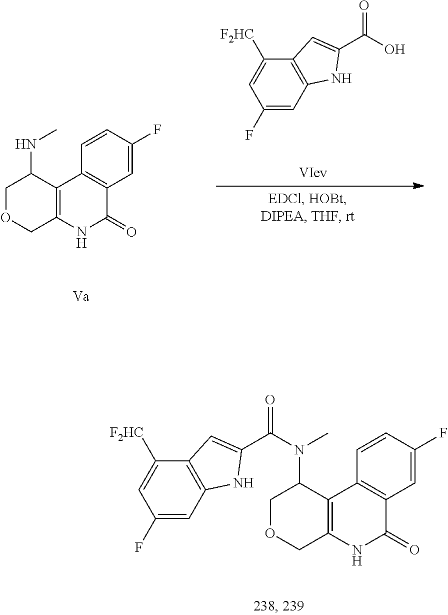 [SMILES: CNC1COCc2[nH]c(=O)c3cc(F)ccc3c21 ; O=C(O)c1cc2c(C(F)F)cc(F)cc2[nH]1 ; CN(C(=O)c1cc2c(C(F)F)cc(F)cc2[nH]1)C1COCc2[nH]c(=O)c3cc(F)ccc3c21]

Racemic 4-(difluoromethyl)-6-fluoro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from racemic 8-fluoro-1-(methylamino)-1,5-dihydro-2H-pyrano[3,4-c]isoquinolin-6(4H)-one (Va) and 4-(difluoromethyl)-6-fluoro-1H-indole-2-carboxylic acid (VIcv). The enantiomers were subsequently separated by preparative SFC: Method isocratic, Mobile phase MeOH:CO₂—50:50. Column: Chiralpak-IA (30×250 mm), 5μ, flow rate: 110 g/min.

Enantiomer I (Compound 238): LCMS: m/z found 460.3 [M+H]⁺, RT=6.74 min, (Method A); ¹H NMR (400 MHz, DMSO-d₆) δ 11.96 (bs, 2H), 7.87 (d, 1H), 7.58-7.48 (m, 2H), 7.44-7.30 (m, 3H), 7.01 (s, 1H), 5.74 (s, 1H), 4.58 (d, 1H), 4.44 (d, 1H), 4.16 (d, 1H), 4.03 (d, 1H), 3.13 (s, 3H); Chiral analytical SFC: RT=2.30 min, Column: Chiralcel IA-3 (4.6×150 mm) 3 μm, 30% MeOH, Flow rate: 3.0 g/min.

Enantiomer II (Compound 239): LCMS: m/z found 460.3 [M+H]⁺, RT=6.74 min, (Method A); ¹H NMR (400 MHz, DMSO-d₆) δ 11.96 (bs, 2H), 7.87 (d, 1H), 7.58-7.48 (m, 2H), 7.44-7.30 (m, 3H), 7.01 (s, 1H), 5.74 (s, 1H), 4.58 (d, 1H), 4.44 (d, 1H), 4.16 (d, 1H), 4.03 (d, 1H), 3.13 (s, 3H); Chiral analytical SFC: RT=5.35 min, Column: Chiralcel IA-3 (4.6×150 mm) 3 μm, 30% MeOH, Flow rate: 3.0 g/min.

### 4-(Difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide (Compounds 255 and 256)

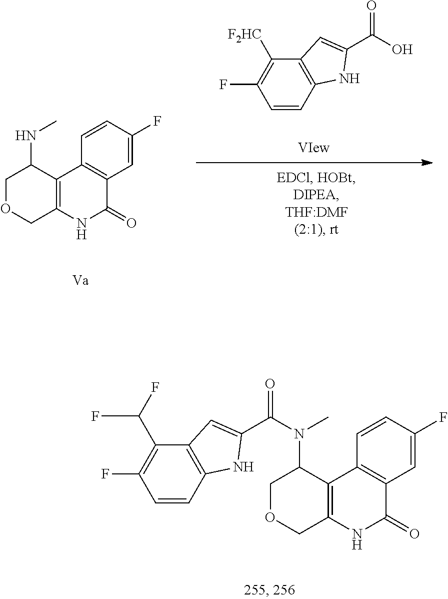 [SMILES: O=C(O)c1cc2c(C(F)F)c(F)ccc2[nH]1 ; CNC1COCc2[nH]c(=O)c3cc(F)ccc3c21 ; CN(C(=O)c1cc2c(C(F)F)c(F)ccc2[nH]1)C1COCc2[nH]c(=O)c3cc(F)ccc3c21]

Racemic 4-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from racemic 8-fluoro-1-(methylamino)-1,5-dihydro-2H-pyrano[3,4-c]isoquinolin-6(4H)-one (Va) and 4-(difluoromethyl)-5-fluoro-1H-indole-2-carboxylic acid (VIcw). The enantiomers were subsequently separated by preparative SFC: Method isocratic, Mobile phase MeOH:CO₂—50:50. Column: Chiralpak-IA (30×250 mm), 5 μm, flow rate: 100 g/min.

Enantiomer I (Compound 255): LCMS: m/z found 460.3 [M+H]⁺, RT=7.57 min, (Method A); ¹H NMR (400 MHz, DMSO-d₆) δ 12.14 (bs, 1H), 11.70 (bs, 1H), 7.92-7.90 (m, 1H), 7.66-7.37 (m, 4H), 7.21-7.16 (m, 1H), 7.02 (s, 1H), 5.75 (s, 1H), 4.65 (d, 1H), 4.50 (d, 1H), 4.21 (d, 1H), 4.07 (d, 1H), 3.12 (s, 3H); Chiral analytical SFC: RT=2.19 min, Column: Chiralcel IA-3 (4.6×150 mm) 3 μm, 40% (Methanol:ACN (1:1)), Flow rate: 3.0 g/min.

Enantiomer II (Compound 256): LCMS: m/z found 460.3 [M+H]⁺, RT=7.57 min, (Method A); ¹H NMR (400 MHz, DMSO-d₆) δ 12.14 (bs, 1H), 11.70 (bs, 1H), 7.92-7.90 (m, 1H), 7.66-7.37 (m, 4H), 7.21-7.16 (m, 1H), 7.02 (s, 1H), 5.75 (s, 1H), 4.65 (d, 1H), 4.50 (d, 1H), 4.21 (d, 1H), 4.07 (d, 1H), 3.12 (s, 3H); Chiral analytical SFC: RT=4.23 min, Column: Chiralcel IA-3 (4.6×150 mm) 3 μm, 40% (Methanol:ACN (1:1)), Flow rate: 3.0 g/min.

### (S)-N-(8,9-Difluoro-6-oxo-1,4,5,6-tetrahydro-2H-pyrano[3,4-c]isoquinolin-1-yl)-2-hydroxy-N-methyl-2,2-diphenylacetamide (Compound 229)

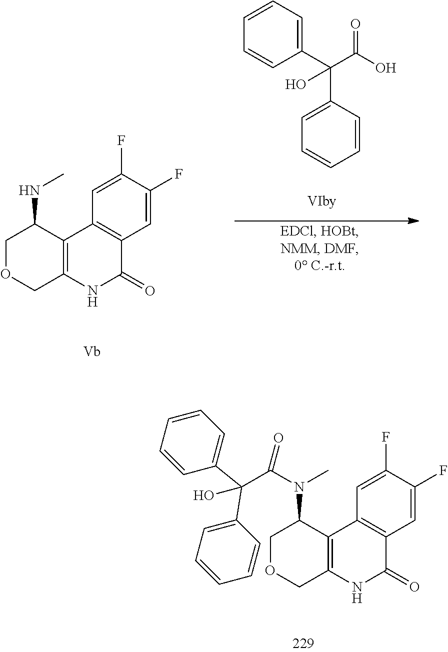 [SMILES: CN[C@@H]1COCc2[nH]c(=O)c3cc(F)c(F)cc3c21 ; O=C(O)C(O)(c1ccccc1)c1ccccc1 ; CN(C(=O)C(O)(c1ccccc1)c1ccccc1)[C@@H]1COCc2[nH]c(=O)c3cc(F)c(F)cc3c21]

A stirred mixture of enantiomerically pure (S)-8,9-difluoro-1-(methylamino)-1,5-dihydro-2H-pyrano[3,4-c]isoquinolin-6(4H)-one (Vb, 40 mg, 0.15 mmol), 2-hydroxy-2,2-diphenylacetic acid (VIby, 34 mg, 0.15 mmol), 3-(ethyliminomethyleneamino)-N,N-dimethyl-propan-1-amine hydrochloride (EDCI, 32 mg, 0.17 mmol), and 1-hydroxybenzotriazole (HOBt, 23 mg, 0.17 mmol) in DMF (0.5 mL) was treated at 0° C. with 4-methylmorpholine (33 μL, 2 eq.), and then the reaction was continued for 4 h allowing it to warm to room temperature. The reaction mixture was partitioned between ethyl acetate and water and the organic extract was dried (sodium sulfate), filtered, and the volatiles evaporated. The product was isolated by preparative reverse phase hplc (Gilson, C₁₈ column, water/acetonitrile 0-75%, with 0.05% v/v FA as a modifier), followed by lyophilization to afford (S)-N-(8,9-difluoro-6-oxo-1,4,5,6-tetrahydro-2H-pyrano[3,4-c]isoquinolin-1-yl)-2-hydroxy-N-methyl-2,2-diphenylacetamide. LCMS m/z found 477.3 [M+H]⁺; RT=5.54 min (Method A); ¹H NMR (400 MHz, DMSO-d₆) δ 11.63 (s, 1H), 8.07 (dd, 1H), 7.48-7.17 (m, 10H), 6.94 (s, 1H), 5.58-5.52 (m, 1H), 4.48 (d, 1H), 4.37 (d, 1H), 4.09 (d, 1H), 3.92 (dd, 1H), 2.59 (s, 3H).

### (S)-N-(8,9-Difluoro-6-oxo-1,4,5,6-tetrahydro-2H-pyrano[3,4-c]isoquinolin-1-yl)-N-methylbenzo[d]thiazole-2-carboxamide (Compound 123)

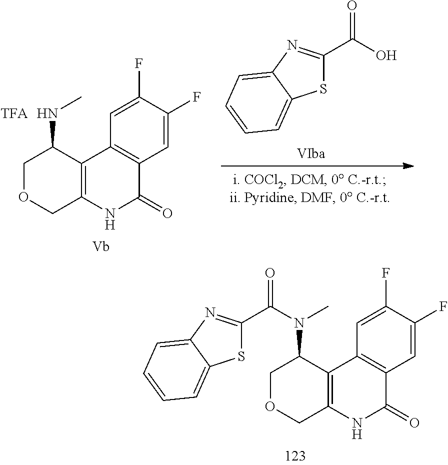

To a stirred solution of benzo[d]thiazole-2-carboxylic acid (VIba) (50 mg, 0.28 mmol) in DCM (2 mL) was added DMF (cat.) followed by dropwise addition of oxalyl chloride (105 mg, 0.84 mmol) at 0° C. The reaction mixture was stirred at room temperature for 2 h. The reaction mixture was then concentrated under reduced pressure. The residue was dissolved in DMF (2 mL), cooled to 0° C., and pyridine (110 mg, 1.35 mmol) added followed by enantiomerically pure (S)-8,9-difluoro-1-(methylamino)-1,5-dihydro-2H-pyrano[3,4-c]isoquinolin-6(4H)-one (Vb, TFA salt, 74 mg, 0.279 mmol). The reaction mixture was then stirred at room temperature for 1 h. The reaction mixture was recooled to 0° C. and water (5 mL) was added. The precipitated solid was collected by filtration, dried under vacuum and subsequently purified by flash column chromatography using 2% MeOH-DCM as eluent to afford (S)-N-(8,9-difluoro-6-oxo-1,4,5,6-tetrahydro-2H-pyrano[3,4-c]isoquinolin-1-yl)-N-methylbenzo[d]thiazole-2-carboxamide (23 mg, 19%). LCMS m/z found 428.1 [M+H]⁺; RT=5.69 min (Method A); ¹H NMR (400 MHz, DMSO-d₆) δ 11.75 (s, 1H), 8.28-8.22 (m, 1H), 8.16-8.11 (m, 2H), 7.63-7.33 (m, 3H), 6.39-5.69 (m, 1H), 4.64 (d, 1H), 4.52-4.45 (m, 1H), 4.34-4.26 (m, 1H), 4.07-4.03 (m, 1H), 3.329 (s, 2H), 2.9 (s, 1H).

### (S)-N-(8,9-Difluoro-6-oxo-1,4,5,6-tetrahydro-2H-pyrano[3,4-c]isoquinolin-1-yl)-N-methylbenzo[d]oxazole-2-carboxamide (Compound 124)

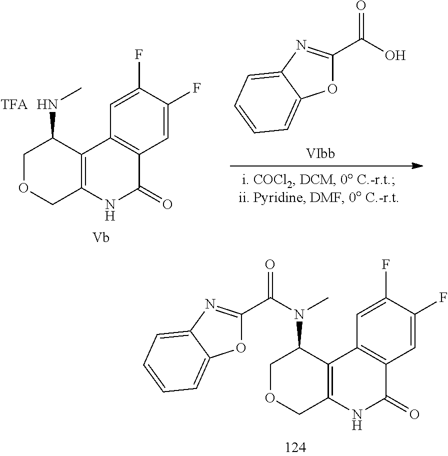

Enantiomerically pure (S)-N-(8,9-difluoro-6-oxo-1,4,5,6-tetrahydro-2H-pyrano[3,4-c]isoquinolin-1-yl)-N-methylbenzo[d]oxazole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and benzo[d]oxazole-2-carboxylic acid (VIbb). LCMS m/z found 412.2 [M+H]⁺; RT=5.24 min (Method A); ¹H NMR (400 MHz, DMSO-d₆) δ 11.75 (s, 1H), 8.168-8.10 (m, 1H), 7.94-7.88 (m, 2H), 7.69-7.33 (m, 3H), 5.87-5.69 (m, 1H), 4.65-4.60 (m, 1H), 4.52-4.44 (m, 1H), 4.36-4.22 (m, 1H), 4.07-4.04 (m, 1H), 3.16 (s, 2H), 2.88 (s, 1H).

### (S)-N-(8,9-Difluoro-6-oxo-1,4,5,6-tetrahydro-2H-pyrano[3,4-c]isoquinolin-1-yl)-N,1-dimethyl-1H-pyrazole-4-carboxamide (Compound 188)

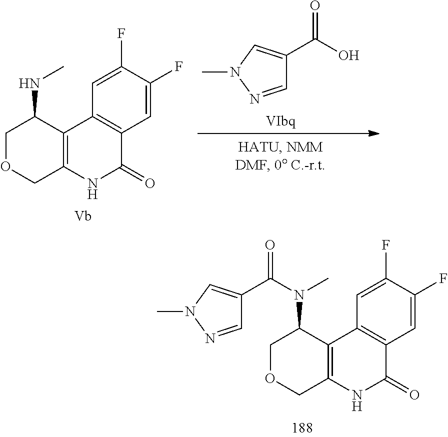

Enantiomerically pure (S)-N-(8,9-difluoro-6-oxo-1,4,5,6-tetrahydro-2H-pyrano[3,4-c]isoquinolin-1-yl)-N,1-dimethyl-1H-pyrazole-4-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 1-methyl-1H-pyrazole-4-carboxylic acid (VIbq). LCMS m/z found 375.2 [M+H]⁺; RT=2.38 min (Method A); ¹H NMR (400 MHz, DMSO-d₆) δ 11.67 (s, 1H), 8.18 (s, 1H), 8.08 (dd, 1H), 7.79 (s, 1H), 7.35 (dd, 1H), 5.65 (s, 1H), 4.59 (d, 1H), 4.43 (dd, H), 4.05 (d, 1H), 3.97 (dd, 1H), 3.84 (s, 3H), 2.97 (s, 3H).

### (S)-5-Bromo-N-(8,9-difluoro-6-oxo-1,4,5,6-tetrahydro-2H-pyrano[3,4-c]isoquinolin-1-yl)-N-methylthiophene-3-carboxamide (Compound 194)

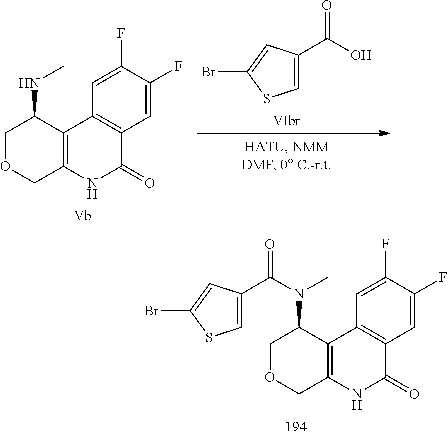

Enantiomerically pure (S)-5-bromo-N-(8,9-difluoro-6-oxo-1,4,5,6-tetrahydro-2H-pyrano[3,4-c]isoquinolin-1-yl)-N-methylthiophene-3-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5-bromothiophene-3-carboxylic acid (VIbr). LCMS m/z found 455.1 [M+H]⁺; RT=4.20 min (Method A); ¹H NMR (400 MHz, DMSO-d₆) δ 11.69 (s, 1H), 8.10 (dd, 1H), 7.87 (d, 1H), 7.50-7.25 (m, 2H), 5.59 (s, 1H), 4.57 (d, 1H), 4.43 (d, 1H), 4.16 (d, 1H), 4.06-3.93 (m, 1H), 2.82 (s, 3H).

### (S)-N-(8,9-Difluoro-6-oxo-1,4,5,6-tetrahydro-2H-pyrano[3,4-c]isoquinolin-1-yl)-N-methyl-2,3-dihydro-1H-indene-2-carboxamide (Compound 195)

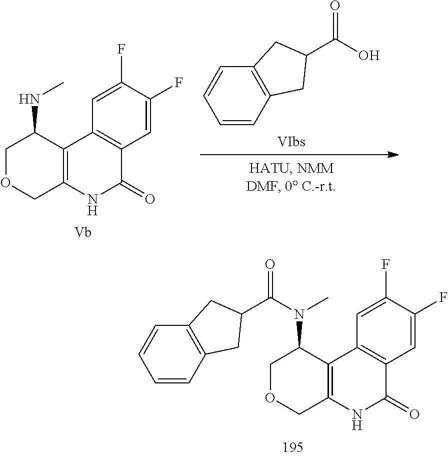

Enantiomerically pure (S)-N-(8,9-difluoro-6-oxo-1,4,5,6-tetrahydro-2H-pyrano[3,4-c]isoquinolin-1-yl)-N-methyl-2,3-dihydro-1H-indene-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 2,3-dihydro-1H-indene-2-carboxylic acid (VIbs). LCMS m/z found 411.3 [M+H]⁺; RT=4.42 min (Method A); ¹H NMR (400 MHz, DMSO-d₆) δ 11.67 (s, 1H), 8.09 (dd, 1H), 7.32-6.99 (m, 5H), 5.72-5.46 (m, 1H), 4.58 (d, 1H), 4.41 (d, 1H), 4.07-3.97 (m, 1H), 3.92 (dd, 1H), 3.76-3.59 (m, 1H), 3.26 (dd, 1H), 3.14 (dd, 2H), 3.02-2.92 (m, 1H), 2.90 (s, 3H).

### (S)-1-(tert-Butyl)-N-(8,9-difluoro-6-oxo-1,4,5,6-tetrahydro-2H-pyrano[3,4-c]isoquinolin-1-yl)-N-methyl-1H-pyrazole-4-carboxamide (Compound 196)

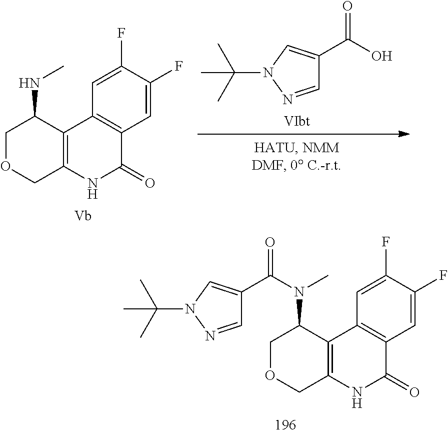

Enantiomerically pure (S)-1-(tert-butyl)-N-(8,9-difluoro-6-oxo-1,4,5,6-tetrahydro-2H-pyrano[3,4-c]isoquinolin-1-yl)-N-methyl-1H-pyrazole-4-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 1-(tert-butyl)-1H-pyrazole-4-carboxylic acid (VIbt). LCMS m/z found 417.2 [M+H]⁺; RT=3.52 min (Method A); ¹H NMR (400 MHz, DMSO-d₆) δ 11.69 (s, 1H), 8.20 (s, 1H), 8.09 (t, 1H), 7.81 (s, 1H), 7.38 (dd, 1H), 5.67 (s, 1H), 4.59 (d, 1H), 4.43 (d, 1H), 4.05 (d, 1H), 3.96 (d, 1H), 2.99 (s, 3H), 1.51 (s, 9H).

### (S)-N-(8,9-Difluoro-6-oxo-1,4,5,6-tetrahydro-2H-pyrano[3,4-c]isoquinolin-1-yl)-N-methyl-1-(trifluoromethyl)-1H-pyrazole-4-carboxamide (Compound 197)

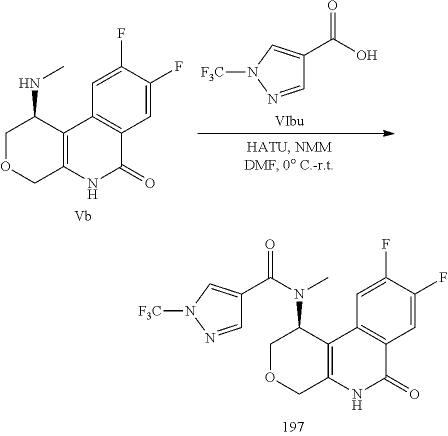

Enantiomerically pure (S)-N-(8,9-difluoro-6-oxo-1,4,5,6-tetrahydro-2H-pyrano[3,4-c]isoquinolin-1-yl)-N-methyl-1-(trifluoromethyl)-1H-pyrazole-4-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 1-(trifluoromethyl)-1H-pyrazole-4-carboxylic acid (VIbu). LCMS m/z found 429.2 [M+H]⁺; RT=3.63 min (Method A); ¹H NMR (400 MHz, DMSO-d₆) δ 11.70 (s, 1H), 8.94 (s, 1H), 8.28 (s, 1H), 8.10 (dd, 1H), 7.35 (dd, 1H), 5.64 (s, 1H), 4.60 (d, 1H), 4.44 (d, 1H), 4.12 (d, 1H), 3.98 (dd, 1H), 2.97 (s, 3H).

### (S)-4-Bromo-N-(8,9-difluoro-6-oxo-1,4,5,6-tetrahydro-2H-pyrano[3,4-c]isoquinolin-1-yl)-N-methyl-1H-pyrrole-2-carboxamide (Compound 198)

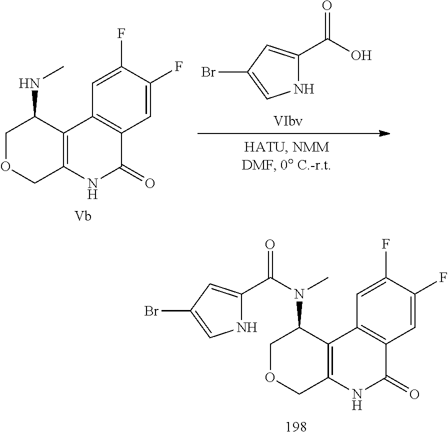

Enantiomerically pure (S)-4-bromo-N-(8,9-difluoro-6-oxo-1,4,5,6-tetrahydro-2H-pyrano[3,4-c]isoquinolin-1-yl)-N-methyl-1H-pyrrole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-bromo-1H-pyrrole-2-carboxylic acid (VIbv). LCMS m/z found 440.1 [M+H]⁺; RT=4.04 min (Method A); ¹H NMR (400 MHz, DMSO-d₆) δ 12.02 (s, 1H), 11.72 (s, 1H), 8.16-8.06 (m, 1H), 7.39 (t, 1H), 7.11 (d, 1H), 6.71 (s, 1H), 5.69 (s, 1H), 4.62 (d, 1H), 4.45 (dd, Hz, 1H), 4.08 (d, 1H), 3.99 (d, J=12.0 Hz, 1H), 3.04 (s, 3H).

### (S)-N-(8,9-Difluoro-6-oxo-1,4,5,6-tetrahydro-2H-pyrano[3,4-c]isoquinolin-1-yl)-1-hydroxy-N-methylcyclohexane-1-carboxamide (Compound 211)

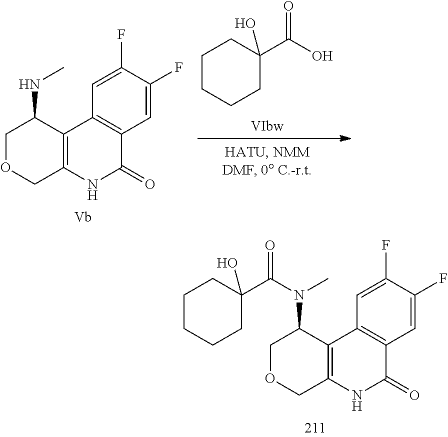

Enantiomerically pure (S)-N-(8,9-difluoro-6-oxo-1,4,5,6-tetrahydro-2H-pyrano[3,4-c]isoquinolin-1-yl)-1-hydroxy-N-methylcyclohexane-1-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 1-hydroxycyclohexane-1-carboxylic acid (VIbw). LCMS m/z found 393.3 [M+H]⁺; RT=4.03 min (Method A); ¹H NMR (400 MHz, DMSO-d₆) δ 11.66 (s, 1H), 8.08 (ddd, 1H), 7.16 (dd, 1H), 5.53 (s, 1H), 5.28 (s, 1H), 4.53 (d, 1H), 4.39 (d, 1H), 3.99-3.86 (m, 2H), 3.08-3.02 (m, 3H), 1.83 (d, 1H), 1.78-1.34 (m, 8H), 1.22 (d, 1H).

### (S)-N-(8,9-Difluoro-6-oxo-1,4,5,6-tetrahydro-2H-pyrano[3,4-c]isoquinolin-1-yl)-N-methyl-2-(trifluoromethyl)-1H-imidazole-5-carboxamide (Compound 225)

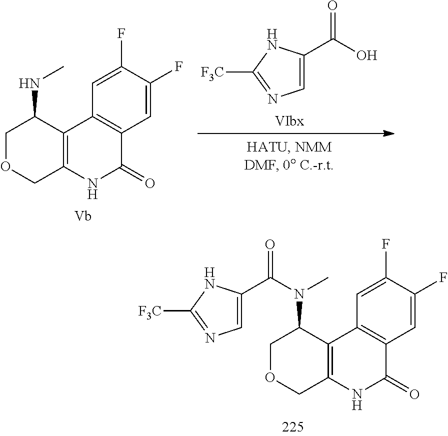

Enantiomerically pure (S)-N-(8,9-difluoro-6-oxo-1,4,5,6-tetrahydro-2H-pyrano[3,4-c]isoquinolin-1-yl)-N-methyl-2-(trifluoromethyl)-1H-imidazole-5-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 2-(trifluoromethyl)-1H-imidazole-5-carboxylic acid (VIbx). LCMS m/z found 429.2 [M+H]⁺; RT=3.60 min (Method A); ¹H NMR (400 MHz, DMSO-d₆) δ 11.69 (d, 1H), 8.14-8.04 (m, 1H), 7.98 (s, 1H), 7.66 (m, 1H)*, 7.34 (dd, 1H), 6.54 (s, 1H)*, 6.01 (s, 1H), 5.67 (t, 1H), 4.58 (dd, 1H), 4.43 (dd, 1H), 4.09 (d, 1H), 3.98 (ddd, 1H), 3.12 (s, 3H), 2.73 (s, 3H)*. NOTE: “*” denotes minor rotamer or tautomer.

### (S)-N-(8,9-Difluoro-6-oxo-1,4,5,6-tetrahydro-2H-pyrano[3,4-c]isoquinolin-1-yl)-5,6-difluoro-N-methyl-2,3-dihydro-1H-indene-2-carboxamide (Compound 233)

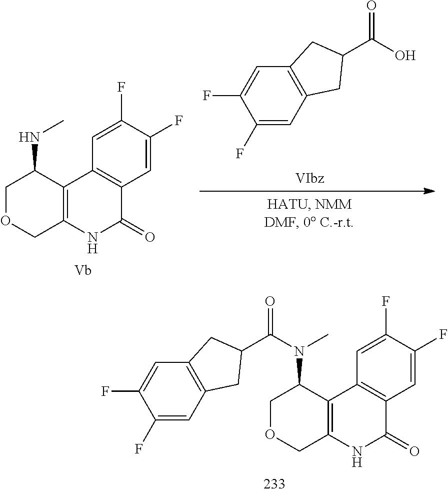

Enantiomerically pure (S)-N-(8,9-difluoro-6-oxo-1,4,5,6-tetrahydro-2H-pyrano[3,4-c]isoquinolin-1-yl)-5,6-difluoro-N-methyl-2,3-dihydro-1H-indene-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5,6-difluoro-2,3-dihydro-1H-indene-2-carboxylic acid (VIbz). LCMS m/z found 447.3 [M+H]⁺; RT=5.70 min (Method A); ¹H NMR (400 MHz, DMSO-d₆) δ 11.67 (s, 1H), 8.08 (dd, 1H), 7.25 (ddd, 2H), 7.15 (dd, 1H), 5.60-5.54 (m, 1H), 4.57 (d, 1H), 4.45-4.36 (m, 1H), 4.00 (d, 1H), 3.92 (dd, 1H), 3.80-3.67 (m, 1H), 3.22 (dd, 1H), 3.12 (dd, 2H), 2.98-2.89 (m, 1H), 2.87 (d, 3H).

### (S)-N-(8,9-Difluoro-6-oxo-1,4,5,6-tetrahydro-2H-pyrano[3,4-c]isoquinolin-1-yl)-1-(4-fluorophenyl)-N-methyl-1H-pyrazole-4-carboxamide (Compound 234)

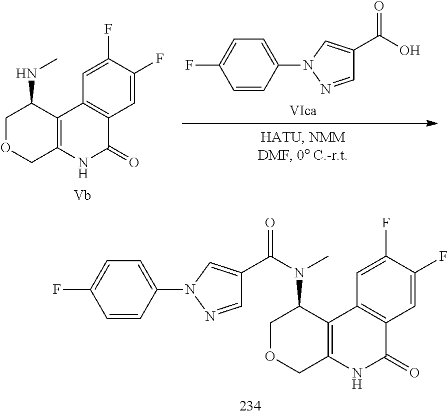

Enantiomerically pure (S)-N-(8,9-difluoro-6-oxo-1,4,5,6-tetrahydro-2H-pyrano[3,4-c]isoquinolin-1-yl)-1-(4-fluorophenyl)-N-methyl-1H-pyrazole-4-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 1-(4-fluorophenyl)-1H-pyrazole-4-carboxylic acid (VIca). LCMS m/z found 455.3 [M+H]⁺; RT=5.40 min (Method A); ¹H NMR (400 MHz, DMSO-d₆) δ 11.71 (s, 1H), 8.90 (s, 1H), 8.15-8.05 (m, 2H), 7.98-7.88 (m, 2H), 7.44-7.30 (m, 3H), 5.68 (t, 1H), 4.61 (d, 1H), 4.45 (dd, 1H), 4.10 (d, 1H), 3.99 (dd, 1H), 3.04 (s, 3H).

### (S)-N-(8,9-Difluoro-6-oxo-1,4,5,6-tetrahydro-2H-pyrano[3,4-c]isoquinolin-1-yl)-5-(4-fluorophenyl)-N-methyl-1,2,4-oxadiazole-3-carboxamide (Compound 235)

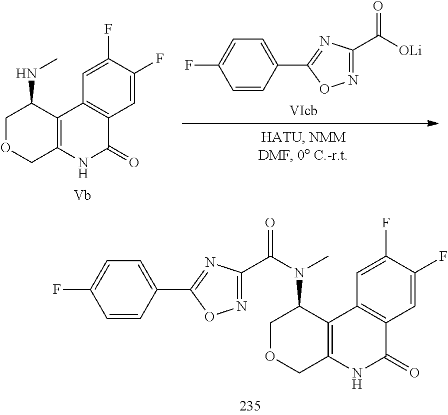

Enantiomerically pure (S)-N-(8,9-difluoro-6-oxo-1,4,5,6-tetrahydro-2H-pyrano[3,4-c]isoquinolin-1-yl)-5-(4-fluorophenyl)-N-methyl-1,2,4-oxadiazole-3-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and lithium 5-(4-fluorophenyl)-1,2,4-oxadiazole-3-carboxylate (VIcb). LCMS m/z found 457.3 [M+H]⁺; RT=5.70 min (Method A); ¹H NMR (400 MHz, DMSO-d₆) δ 11.75 (s, 1H), 8.28-8.05 (m, 3H), 7.63 (dd, 1H)*, 7.59-7.43 (m, 2H), 7.31 (dd, 1H), 5.69-5.63 (m, 1H), 4.97 (s, 1H)*, 4.65-4.54 (m, 1H), 4.52-4.38 (m, 1H), 4.20 (d, 1H), 4.17-4.09 (m, 1H)*, 4.04 (dd, 1H), 3.86 (dd, 1H)*, 2.87 (s, 3H), 2.84 (s, 3H)*. NOTE: “*” denotes distinguishable signals of minor atropisomer.

### (S)-N-(8,9-Difluoro-6-oxo-1,4,5,6-tetrahydro-2H-pyrano[3,4-c]isoquinolin-1-yl)-3-(4-fluorophenyl)-N-methylisoxazole-5-carboxamide (Compound 236)

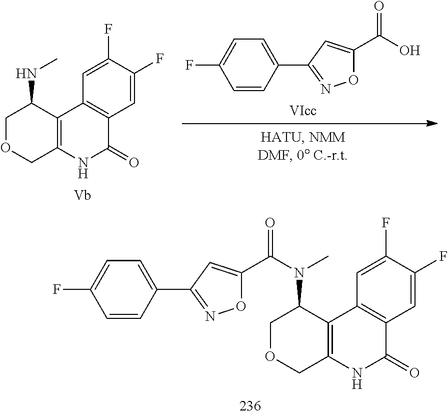

Enantiomerically pure ((S)-N-(8,9-difluoro-6-oxo-1,4,5,6-tetrahydro-2H-pyrano[3,4-c]isoquinolin-1-yl)-3-(4-fluorophenyl)-N-methylisoxazole-5-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-(4-fluorophenyl)isoxazole-5-carboxylic acid (VIcc). LCMS m/z found 456.2 [M+H]⁺; RT=5.89 min (Method A); ¹H NMR (400 MHz, DMSO-d₆) δ 11.75 (s, 1H), 8.12 (dd, 1H), 8.07-7.94 (m, 2H), 7.67 (d, 1H), 7.44-7.27 (m, 3H), 5.65-5.59 (m, 1H), 4.62 (d, 1H), 4.47 (d, 1H), 4.20 (d, 1H), 4.01 (dd, 1H), 2.99 (d, 3H).

### (S)-N-(8,9-Difluoro-6-oxo-1,4,5,6-tetrahydro-2H-pyrano[3,4-c]isoquinolin-1-yl)-4-(3-fluorophenoxy)-N-methylbenzamide (Compound 237)

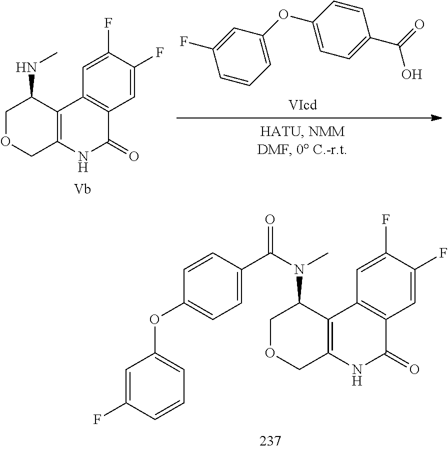

Enantiomerically pure (S)-N-(8,9-difluoro-6-oxo-1,4,5,6-tetrahydro-2H-pyrano[3,4-c]isoquinolin-1-yl)-4-(3-fluorophenoxy)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-(3-fluorophenoxy)benzoic acid (VIcd). LCMS m/z found 481.3 [M+H]⁺; RT=6.22 min (Method A); ¹H NMR (400 MHz, DMSO-d₆) δ 11.69 (s, 1H), 8.10 (dd, 1H), 7.43 (tt, 4H), 7.13-7.05 (m, 2H), 7.09-6.91 (m, 2H), 6.88 (dd, 1H), 5.65 (t, 1H), 4.57 (d, 1H), 4.44 (d, 1H), 4.23 (d, 1H), 4.02 (dd, 1H), 2.73 (s, 3H).

### (S)-N-(8,9-Difluoro-6-oxo-1,4,5,6-tetrahydro-2H-pyrano[3,4-c]isoquinolin-1-yl)-4-(3-fluorophenoxy)-N-methylbenzamide (Compound 249)

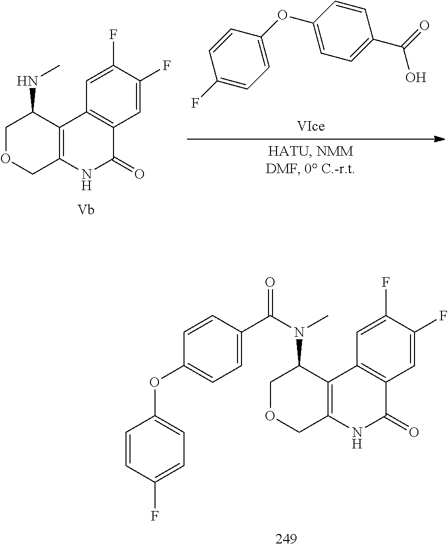

Enantiomerically pure (S)-N-(8,9-difluoro-6-oxo-1,4,5,6-tetrahydro-2H-pyrano[3,4-c]isoquinolin-1-yl)-4-(3-fluorophenoxy)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4-(4-fluorophenoxy)benzoic acid (VIce). LCMS m/z found 481.3 [M+H]⁺; RT=6.63 min (Method A); ¹H NMR (400 MHz, DMSO-d₆) δ 11.69 (s, 1H), 8.10 (dd, 1H), 7.50-7.38 (m, 3H), 7.24 (t, 2H), 7.19-7.08 (m, 2H), 7.03-6.95 (m, 2H), 5.64 (t, 1H), 4.56 (d, 1H), 4.44 (d, 1H), 4.22 (d, 1H), 4.01 (dd, 1H), 2.73 (s, 3H).

### (S)-N-(8,9-Difluoro-6-oxo-1,4,5,6-tetrahydro-2H-pyrano[3,4-c]isoquinolin-1-yl)-4′-fluoro-N-methyl-[1,1′-biphenyl]-4-carboxamide (Compound 250)

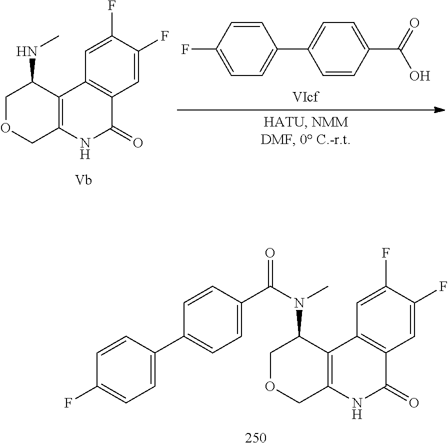

Enantiomerically pure (S)-N-(8,9-difluoro-6-oxo-1,4,5,6-tetrahydro-2H-pyrano[3,4-c]isoquinolin-1-yl)-4′-fluoro-N-methyl-[1,1′-biphenyl]-4-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4′-fluoro-[1,1′-biphenyl]-4-carboxylic acid (VIcf). LCMS m/z found 465.3 [M+H]⁺; RT=6.62 min (Method A); ¹H NMR (400 MHz, DMSO-d₆) δ 11.70 (s, 1H), 8.12 (dd, 1H), 7.78-7.68 (m, 4H), 7.54-7.43 (m, 3H), 7.35-7.24 (m, 2H), 5.71-5.65 (m, 1H), 4.58 (d, 1H), 4.45 (d, 1H), 4.26 (d, 1H), 4.03 (dd, 1H), 2.74 (s, 3H).

### (S)-N-(8,9-Difluoro-6-oxo-1,4,5,6-tetrahydro-2H-pyrano[3,4-c]isoquinolin-1-yl)-2,4′-difluoro-N-methyl-[1,1′-biphenyl]-4-carboxamide (Compound 262)

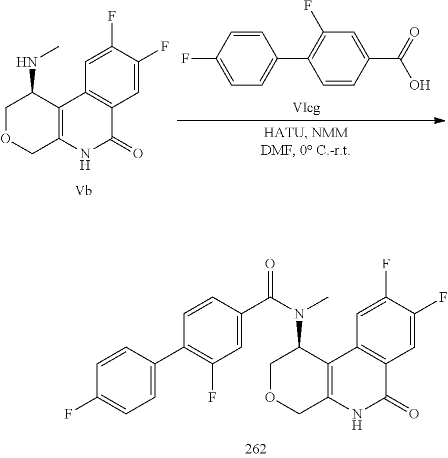

Enantiomerically pure (S)-N-(8,9-difluoro-6-oxo-1,4,5,6-tetrahydro-2H-pyrano[3,4-c]isoquinolin-1-yl)-2,4′-difluoro-N-methyl-[1,1′-biphenyl]-4-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 2,4′-difluoro-[1,1′-biphenyl]-4-carboxylic acid (VIcg). LCMS m/z found 483.3 [M+H]⁺; RT=7.45 min (Method A); ¹H NMR (400 MHz, DMSO-d₆) δ 11.71 (s, 1H), 8.12 (dd, 1H), 7.66-7.55 (m, 3H), 7.52-7.37 (m, 2H), 7.37-7.25 (m, 3H), 5.66 (d, 1H), 4.58 (d, 1H), 4.50-4.41 (m, 1H), 4.28 (d, 1H), 4.02 (dd, 1H), 2.75 (s, 3H).

### (S)-N-(8,9-Difluoro-6-oxo-1,4,5,6-tetrahydro-2H-pyrano[3,4-c]isoquinolin-1-yl)-5-(4-fluorophenyl)-N-methylisoxazole-3-carboxamide (Compound 263)

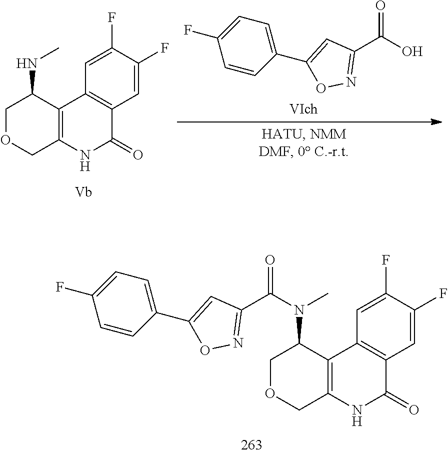

Enantiomerically pure (S)-N-(8,9-difluoro-6-oxo-1,4,5,6-tetrahydro-2H-pyrano[3,4-c]isoquinolin-1-yl)-5-(4-fluorophenyl)-N-methylisoxazole-3-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5-(4-fluorophenyl)isoxazole-3-carboxylic acid (VIch). LCMS m/z found 456.3 [M+H]⁺; RT=7.18 min (Method A); ¹H NMR (400 MHz, DMSO-d₆) δ 11.73 (d, 1H), 8.11 (dt, 1H), 8.07-7.94 (m, 2H), 7.50-7.28 (m, 4H), 5.69-5.63 (m, 1H), 4.65-4.54 (m, 1H), 4.51-4.37 (m, 1H), 4.19 (d, 1H), 4.03 (dd, 1H), 2.92 (s, 3H).

### (S)-N-(8,9-Difluoro-6-oxo-1,4,5,6-tetrahydro-2H-pyrano[3,4-c]isoquinolin-1-yl)-2-hydroxy-N-methyl-2,3-dihydro-1H-indene-2-carboxamide (Compound 275)

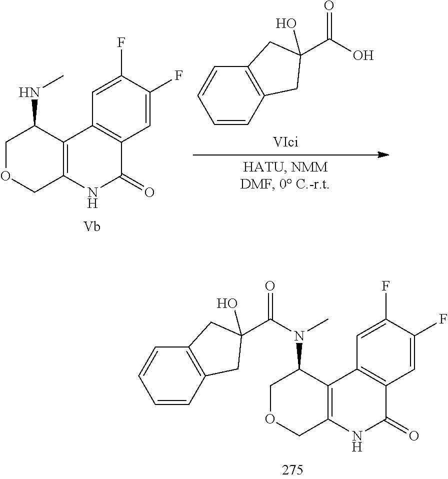

Enantiomerically pure (S)-N-(8,9-difluoro-6-oxo-1,4,5,6-tetrahydro-2H-pyrano[3,4-c]isoquinolin-1-yl)-2-hydroxy-N-methyl-2,3-dihydro-1H-indene-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 2-hydroxy-2,3-dihydro-1H-indene-2-carboxylic acid (VIci). LCMS m/z found 427.3 [M+H]⁺; RT=7.21 min (Method A); ¹H NMR (400 MHz, DMSO-d₆) δ 11.67 (s, 1H), 8.09 (dd, 1H), 7.28-7.08 (m, 5H), 5.93 (s, 1H), 5.51 (t, 1H), 4.56 (d, 1H), 4.41 (d, 1H), 4.02 (d, 1H), 3.92 (dd, 1H), 3.74 (d, 1H), 3.45 (d, 1H), 3.11-3.01 (d, 2H), 3.03 (s, 3H).

### 5,6-Difluoro-2-hydroxy-2,3-dihydro-1H-indene-2-carboxylic acid (VIcj)

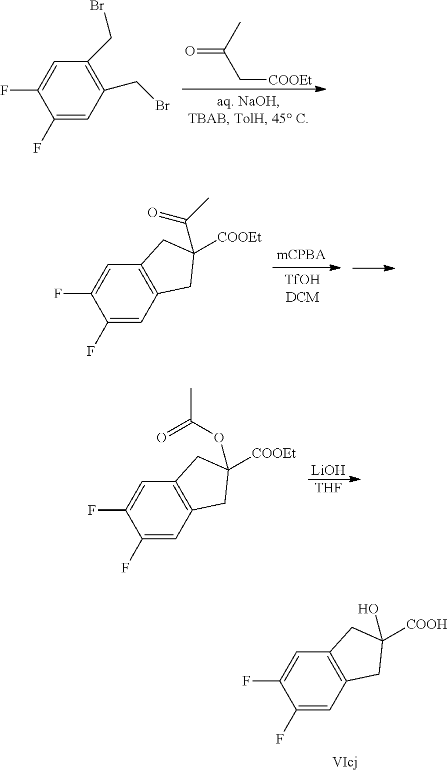

Step i. 1,2-Bis(bromomethyl)-4,5-difluoro-benzene (1.00 g, 3.33 mmol) and ethyl 3-oxobutanoate (0.45 mL, 0.46 g, 3.50 mmol) in toluene (65 mL), at 0-20° C. was treated portionwise with sodium hydroxide 48% in water (1.90 mL, 19.0 mmol) and tetrabutylammonium bromide (54 mg, 0.17 mmol). The mixture was stirred at room temperature for 4 h. The reaction was quenched with saturated ammonium chloride, the layers were separated, and the organic solution was dried (sodium sulfate), filtered, and volatiles evaporated. The product was purified by flash-chromatography (silica gel, EtOAc/hexanes 0-75%) to afford ethyl 2-acetyl-5,6-difluoro-2,3-dihydro-1H-indene-2-carboxylate (530 mg, 59% yield). LCMS m/z found 269.25 [M+H]⁺, RT=1.2 min (Method B); ¹H NMR (400 MHz, CDCl₃) δ 7.01-6.92 (m, 2H), 4.22 (qd, 2H), 3.53-3.38 (m, 4H), 2.23 (d, 3H), 1.27 (td, 3H).

Step ii. Ethyl 2-acetyl-5,6-difluoro-2,3-dihydro-1H-indene-2-carboxylate (530 mg, 1.98 mmol) and trifluoromethanesulfonic acid (87.21 μL, 148 mg, 0.99 mmol) in DCM (10 mL) at room temperature was treated portionwise with 3-methylbenzenecarboperoxoic acid (˜70% in benzoic acid, 2 g, 9.88 mmol). The mixture was stirred at 45° C. for 16 h. The reaction mixture was cooled to room temperature, diluted with 10 mL DCM, filtered, washed with a 30% NaHSO₃ solution, then with a saturated NaHCO₃ solution, dried (MgSO₄), filtered and the solvent was evaporated. The product was isolated by flash-chromatography (silica gel, EtOAc/Hexanes) to afford ethyl 2-acetoxy-5,6-difluoro-2,3-dihydro-1H-indene-2-carboxylate (150 mg, 27% yield). LCMS m/z found 285.2 [M+H]⁺, RT=1.27 min (Method B); ¹H NMR (400 MHz, CDCl₃) δ 7.16-6.91 (m, 2H), 4.22 (qd, 2H), 3.64 (dt, 2H), 3.28 (d, 2H), 2.06 (d, 3H), 1.32-1.20 (m, 3H).

Step iii. Ethyl 2-acetoxy-5,6-difluoro-2,3-dihydro-1H-indene-2-carboxylate (150 mg, 0.53 mmol) in 2 mL of THE was treated at room temperature with lithium hydroxide (63 mg, 2.64 mmol) in water (0.5 mL) for 16 h. The reaction mixture was acidified with 2N HCl to pH-2.5-3 and extracted with ethyl acetate, dried (magnesium sulfate), filtered and the solvent was evaporated. The product was triturated from minimum amount of ethyl acetate and hexane (1:3, v/v), and the solid was dried on high vacuum to afford 5,6-difluoro-2-hydroxy-2,3-dihydro-1H-indene-2-carboxylic acid (VIcj, 78 mg, 69% yield). ¹H NMR (400 MHz, Methanol-d₄) δ 7.10 (t, 2H), 4.91 (s, 2H), 3.50 (dt, 2H), 3.02 (d, 2H).

### (S)-N-(8,9-Difluoro-6-oxo-1,4,5,6-tetrahydro-2H-pyrano[3,4-c]isoquinolin-1-yl)-5,6-difluoro-2-hydroxy-N-methyl-2,3-dihydro-1H-indene-2-carboxamide (Compound 276)

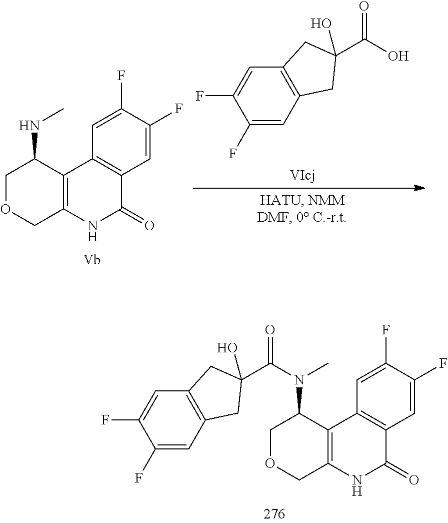

Enantiomerically pure (S)-N-(8,9-difluoro-6-oxo-1,4,5,6-tetrahydro-2H-pyrano[3,4-c]isoquinolin-1-yl)-5,6-difluoro-2-hydroxy-N-methyl-2,3-dihydro-1H-indene-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5,6-difluoro-2-hydroxy-2,3-dihydro-1H-indene-2-carboxylic acid (VIcj). LCMS m/z found 463.3 [M+H]⁺; RT=7.33 min (Method A); ¹H NMR (400 MHz, DMSO-d₆) δ 11.67 (s, 1H), 8.09 (dd, 1H), 7.25 (ddd, 2H), 7.12 (dd, 1H), 6.06 (s, 1H), 5.49 (d, 1H), 4.56 (d, 1H), 4.46-4.36 (m, 1H), 4.01 (d, 1H), 3.91 (dd, 1H), 3.71 (d, 1H), 3.41 (d, 1H), 3.07-2.98 (d, 2H), 3.04 (s, 3H).

### 6-(Difluoromethyl)-5-fluoro-1H-indole-2-carboxylic acid (VIct)

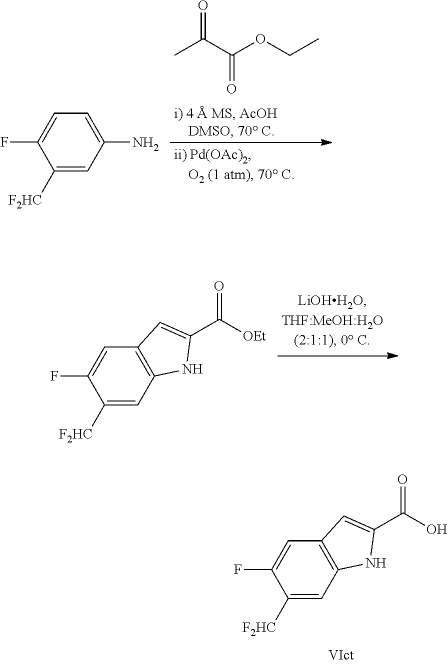

Step i. To a stirred solution of 4 g (24.8 mmol, 1 eq.) of 3-(difluoromethyl)-4-fluoroaniline in 40 mL of DMSO (10 vol) were added 3.74 g (32.3 mmol, 1.3 eq.) of ethyl pyruvate and 1.5 g (24.8 mmol) of AcOH, along with 1 g of activated 4 Å molecular sieves. The reaction mixture was heated to 70° C. for 2 h under nitrogen atmosphere. Next 0.55 g (2.48 mmol, 0.1 eq.) of Pd(OAc)₂ was added at room temperature and the reaction mixture was stirred at 70° C. under an oxygen balloon (1 atm) for 16 h. After completion of the reaction, the mixture was filtered through a pad of CELITE® and the filtrate was diluted with ice-water (100 mL) and extracted with ethyl acetate (2×100 mL). Combined organic layers were washed with ice-water, dried over anhydrous Na₂SO₄ and concentrated under reduced pressure. Obtained material was purified by column chromatography using silica gel (100-200 mesh) and 10% ethyl acetate in petroleum ether as eluent to afford 2.0 g (7.78 mmol, 31% yield) of ethyl 6-(difluoromethyl)-5-fluoro-1H-indole-2-carboxylate. LCMS m/z found 256.22 [M−H]⁻, ¹H NMR (400 MHz, DMSO-d₆) δ 12.31 (s, 1H), 7.68 (d, 1H), 7.61 (d, 1H), 7.39-7.12 (m, 2H), 4.39-4.34 (m, 2H), 1.26 (t, 3H).

Step ii. To a stirred solution of 900 mg (3.5 mmol, 1 eq.) of ethyl 6-(difluoromethyl)-5-fluoro-1H-indole-2-carboxylate (obtained in Step i) in a mixture of THF:Water:MeOH (2:1:1) (9 mL), 367 mg (8.75 mmol, 2.5 eq.) of LiOH—H₂O was added at 0° C. and the reaction was stirred at room temperature for 4 h. After completion of reaction, volatiles were evaporated from reaction mixture and the residue was acidified with 10% aq. KHSO₄ solution to pH-2. The precipitated solids were collected by filtration, washed with water and dried under vacuum to afford 600 mg (2.62 mmol, 75% yield) of 6-(difluoromethyl)-5-fluoro-1H-indole-2-carboxylic acid (VIct). LCMS: m/z found 228.0 [M−H]⁻, RT=2.64 min, (Method C); ¹H NMR (400 MHz, DMSO-d₆) δ 13.29 (bs, 1H), 12.19 (s, 1H), 7.67 (d, 1H), 7.59 (d, 1H), 7.39-7.12 (m, 2H).

### (S)-N-(8,9-Difluoro-6-oxo-1,4,5,6-tetrahydro-2H-pyrano[3,4-c]isoquinolin-1-yl)-6-(difluoromethyl)-5-fluoro-N-methyl-1H-indole-2-carboxamide (Compound 212)

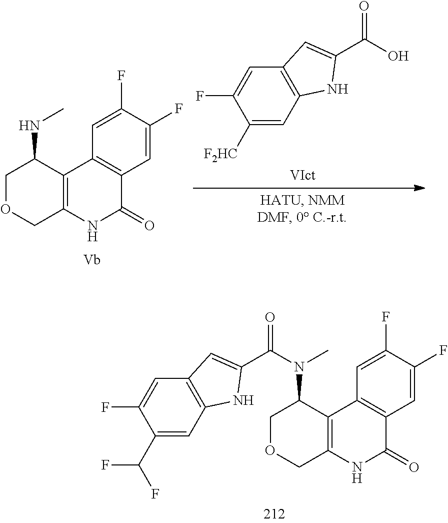

Enantiomerically pure (S)-N-(8,9-difluoro-6-oxo-1,4,5,6-tetrahydro-2H-pyrano[3,4-c]isoquinolin-1-yl)-6-(difluoromethyl)-5-fluoro-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 6-(difluoromethyl)-5-fluoro-1H-indole-2-carboxylic acid (VIct). LCMS m/z found 477.9 [M+H]⁺; RT=4.41 min (Method A); ¹H NMR (400 MHz, DMSO-d₆) δ 12.17 (s, 1H), 11.75 (s, 1H), 8.12 (dd, 1H), 7.70 (d, 1H), 7.54 (d, 1H), 7.48-7.37 (m, 1H), 7.26 (s, 1H), 7.01 (d, 1H), 5.75 (d, 1H), 4.64 (d, 1H), 4.53-4.44 (m, 1H), 4.19 (d, 1H), 4.09-3.97 (m, 1H), 3.16 (s, 3H).

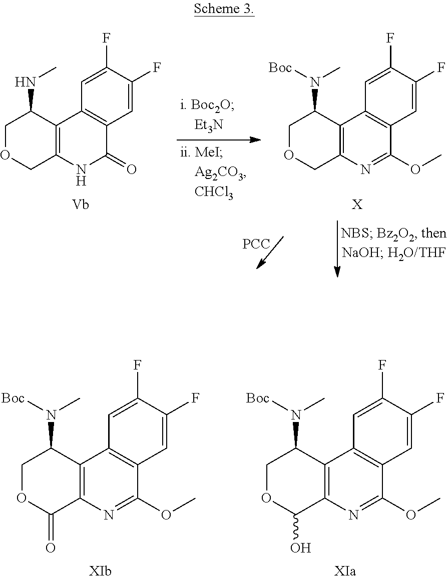

### tert-Butyl ((1S)-8,9-difluoro-4-hydroxy-6-methoxy-1,4-dihydro-2H-pyrano[3,4-c]isoquinolin-1-yl)(methyl)carbamate (X)

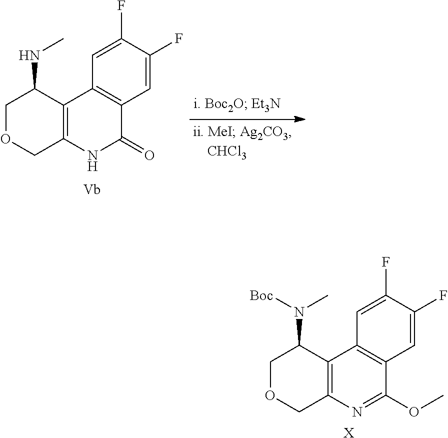

Step i. A solution of (S)-8,9-Difluoro-1-(methylamino)-1,5-dihydro-2H-pyrano[3,4-c]isoquinolin-6(4H)-one (Vb, 480 mg, 1.8 mmol) in 5 mL of THF was treated with triethylamine (0.4 mL, 2.7 mmol), followed by di-tert-butyl dicarbonate (433 mg, 2.0 mmol). After the addition was completed, the reaction was stirred overnight at room temperature under a nitrogen atmosphere. The mixture was diluted with 30 mL of DCM, washed with 0.5 M HCl (20 mL) then with 5% NaHCO₃(20 mL), water (20 mL), and brine (20 mL), dried on magnesium sulfate, filtered, concentrated and the product purified by flash chromatography (silica gel, EtOAc/hexanes 0-10% over 13 min). LCMS m/z found 367.3 [M+H]⁺, RT=0.92 min (Method B); ¹H NMR (400 MHz, CDCl₃) δ 12.52 (s, 1H)*, 12.45 (s, 1H), 8.14 (dd, 1H), 7.47 (dd, 1H), 7.36 (dd, 1H)*, 5.36-5.30 (m, 1H), 5.07-5.02 (m, 1H)*, 4.78 (d, 1H), 4.58 (dd, 1H), 4.28-4.17 (m, 1H), 3.95 (dt, 1H), 2.69 (s, 3H), 1.52 (s, 6H). NOTE: “*” indicates observable signals belonging to the minor carbamate rotamer.

Step ii. tert-Butyl N-methyl-N-[(1S)-8,9-difluoro-6-oxo-1,2,4,5-tetrahydropyrano[3,4-c]isoquinolin-1-yl]carbamate (633 mg, 1.73 mmol) obtained in Step i, iodomethane, (1.4 mL, 22.6 mmol), and silver carbonate (1.2 g, 4.3 mmol) were stirred in chloroform (40 mL) at 45° C., under nitrogen, for 16 h. The reaction mixture was cooled to room temperature, diluted with DCM, and filtered through C.ELITE®. The solvent was evaporated under reduced pressure and the product was purified by flash chromatography (silica gel, EtOAc/Hexanes 0-30%). [Only one regioisomer, consistent with O-methylation, was observed in the crude reaction mixture.] LCMS m/z found 381.3 [M+H]⁺, RT=6.19 min (Method A); ¹H NMR (400 MHz, CDCl₃) δ 7.99 (dt, 1H), 7.56 (ddd, 1H), 5.53-5.47 (m, 1H), 5.25 (s, 1H)*, 4.82 (d, 1H), 4.66 (d, 1H), 4.31-4.19 (m, 1H), 4.08 (s, 3H), 3.97 (ddd, 1H), 2.65 (d, 3H), 1.53 (s, 6H). NOTE: “*” indicates observable signals belonging to minor carbamate rotamer.

### tert-Butyl ((1S)-8,9-difluoro-4-hydroxy-6-methoxy-1,4-dihydro-2H-pyrano[3,4-c]isoquinolin-1-yl)(methyl)carbamate (XIa)

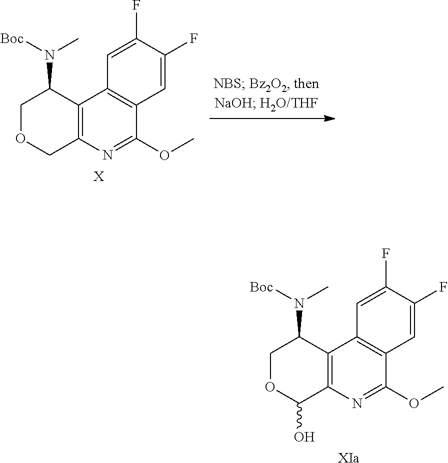

tert-Butyl ((1S)-8,9-difluoro-4-hydroxy-6-methoxy-1,4-dihydro-2H-pyrano[3,4-c]isoquinolin-1-yl)(methyl)carbamate (X, 100 mg, 0.26 mmol) in 5 mL of carbon tetrachloride was treated with 1-bromopyrrolidine-2,5-dione (47 mg, 0.26 mmol) and benzoyl peroxide (3 mg, 0.01 mmol) at 80° C. for 1 h. The reaction mixture was filtered, and solvent was evaporated. The residue was redissolved in THF/water (1:1 v/v, 12 mL) and treated with 1 mL of a 1 M NaOH solution and stirred at 75° C. for 1 h. The reaction mixture was cooled to room temperature and treated with 2 M HCl, followed by saturated sodium bicarbonate to pH 6, and extracted with EtOAc. The organic extracts were dried on sodium sulfate, filtered and the solvent was evaporated. The product was isolated by flash-chromatography (silica gel, EtOAc/Hexanes 0-55%) to afford tert-butyl ((1S)-8,9-difluoro-4-hydroxy-6-methoxy-1,4-dihydro-2H-pyrano[3,4-c]isoquinolin-1-yl)(methyl)carbamate (XIa), as a ˜7:1 mixture of lactol anomers. LCMS m/z found 397.3 [M+H]⁺, RT=5.07 min (major anomer) (Method A). ¹H NMR (400 MHz, CDCl₃) δ 7.99 (dt, 1H), 7.63 (ddd, 1H), 5.97 (s, 1H), 5.48 (d, 1H), 5.21 (d, 1H)*, 4.61 (td, 1H), 4.14 (s, 3H), 4.11-3.99 (m, 1H), 3.85 (s, 1H), 2.58 (s, 3H), 1.52 (s, 6H). NOTE: “*” indicates observable signal belonging to minor carbamate rotamer.

### tert-Butyl (S)-(8,9-difluoro-6-methoxy-4-oxo-1,4-dihydro-2H-pyrano[3,4-c]isoquinolin-1-yl)(methyl)carbamate (XIb)

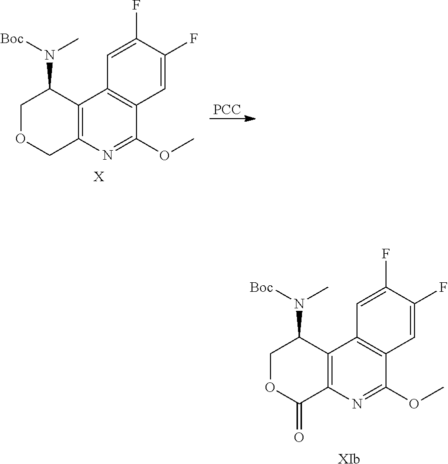

tert-Butyl ((1S)-8,9-difluoro-4-hydroxy-6-methoxy-1,4-dihydro-2H-pyrano[3,4-c]isoquinolin-1-yl)(methyl)carbamate (X, 200 mg, 0.53 mmol) in 10 mL of DCM was treated with pyridinium chlorochromate (453 mg, 2.1 mmol) and the reaction was stirred at 55° C. for 72 h in a sealed tube. The reaction mixture was allowed to cool to room temperature, diluted with DCM and adsorbed onto silicagel. The solvent was evaporated and the residue was dryloaded onto a flash column and the product subsequently eluted (Isco, silica gel, ethyl acetate/hexane 0-45%, 11 min) to afford tert-butyl (S)-(8,9-difluoro-6-methoxy-4-oxo-1,4-dihydro-2H-pyrano[3,4-c]isoquinolin-1-yl)(methyl)carbamate (XIb, Scheme 3). LCMS m/z found 395.2 [M+H]⁺, RT=1.15 min (Method B). ¹H NMR (400 MHz, CDCl₃) δ 8.11 (dd, 1H), 7.97 (dd, 1H), 5.98 (d, 1H), 4.82 (dd, 1H), 4.72 (dd, 1H), 4.25 (s, 3H), 2.68 (s, 3H), 1.52 (s, 9H).

### N-((1S)-8,9-Difluoro-4-hydroxy-6-oxo-1,4,5,6-tetrahydro-2H-pyrano[3,4-c]isoquinolin-1-yl)-5,6-difluoro-N-methyl-1H-indole-2-carboxamide (Compound 187)

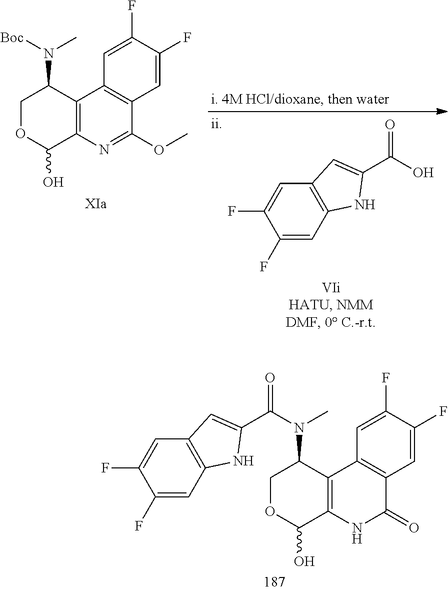 [SMILES: COc1nc2c(c3cc(F)c(F)cc13)[C@H](N(C)C(=O)OC(C)(C)C)COC2O ; O=C(O)c1cc2cc(F)c(F)cc2[nH]1 ; CN(C(=O)c1cc2cc(F)c(F)cc2[nH]1)[C@@H]1COC(O)c2[nH]c(=O)c3cc(F)c(F)cc3c21]

Step i. tert-Butyl ((1S)-8,9-difluoro-4-hydroxy-6-methoxy-1,4-dihydro-2H-pyrano[3,4-c]isoquinolin-1-yl)(methyl)carbamate (XIa, 83 mg, 0.21 mmol) was treated with 4 M hydrogen chloride (3 mL, 12 mmol) in dioxane for 1 h at room temperature. Water (1.3 mL, 71 mmol) was added dropwise and the reaction was continued for 16 h. The volatiles were evaporated, and the residue was azeotroped with toluene, then dried under high vacuum for 1 h. LCMS m/z 283.1 [M+H]⁺, RT=0.45 min (minor anomer: RT=0.50 min) (Method B). The crude material was used directly in the next step without further purification.

Step ii. N-((1S)-8,9-Difluoro-4-hydroxy-6-oxo-1,4,5,6-tetrahydro-2H-pyrano[3,4-c]isoquinolin-1-yl)-5,6-difluoro-N-methyl-1H-indole-2-carboxamide was synthesized in an analogous manner as described above from the crude product of Step i and 5,6-difluoro-1H-indole-2-carboxylic acid (VIi), as a ˜7:1 mixture of 4-position epimers. LCMS m/z found 462.3 [M+H]⁺, 444.3 [M−OH]⁺; RT=4.53 min (minor); 4.59 min (Major) (Method A); ¹H NMR (400 MHz, DMSO-d₆) δ 11.90 (s, 1H), 11.84 (s, 1H), 11.60 (s, 1H)*, 8.13 (ddd, 1H), 7.67-7.56 (m, 1H), 7.59-7.45 (m, 1H), 7.37 (dd, 1H), 7.27 (d, 1H), 6.99-6.91 (m, 1H), 5.80 (m, 1H)*, 5.68 (dd, 2H), 5.57 (d, 1H)*, 4.47 (dd, 1H), 4.11 (d, 1H)*, 3.92 (d, 1H), 3.09 (d, 3H). NOTE: “*” indicates signals tentatively assigned to minor 4-position lactol epimer.

### (S)-N-(8,9-Difluoro-4,6-dioxo-1,4,5,6-tetrahydro-2H-pyrano[3,4-c]isoquinolin-1-yl)-5,6-difluoro-N-methyl-1H-indole-2-carboxamide (Compound 321)

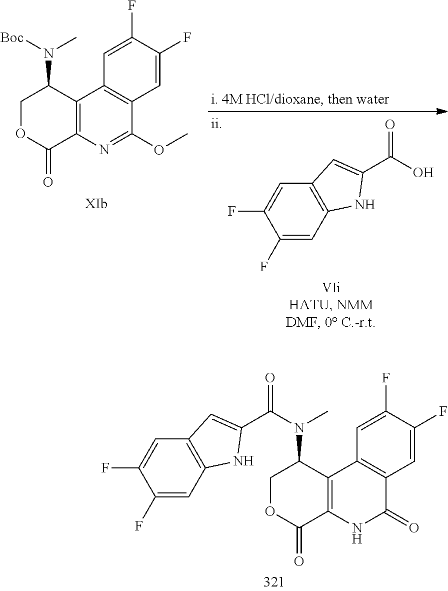 [SMILES: COc1nc2c(c3cc(F)c(F)cc13)[C@H](N(C)C(=O)OC(C)(C)C)COC2=O ; O=C(O)c1cc2cc(F)c(F)cc2[nH]1 ; CN(C(=O)c1cc2cc(F)c(F)cc2[nH]1)[C@@H]1COC(=O)c2[nH]c(=O)c3cc(F)c(F)cc3c21]

Enantiomerically pure (S)-N-(8,9-difluoro-4,6-dioxo-1,4,5,6-tetrahydro-2H-pyrano[3,4-c]isoquinolin-1-yl)-5,6-difluoro-N-methyl-1H-indole-2-carboxamide was synthesized in an analogous manner as described above from enantiomerically pure tert-butyl (S)-(8,9-difluoro-6-methoxy-4-oxo-1,4-dihydro-2H-pyrano[3,4-c]isoquinolin-1-yl)(methyl)carbamate (XIb) and 5,6-difluoro-1H-indole-2-carboxylic acid (VIi). LCMS m/z found 460.1 [M+H]⁺, RT=4.32 min (Method A); ¹H NMR (400 MHz, DMSO-d₆) δ 11.96 (s, 1H), 11.84 (s, 1H), 8.28-8.19 (m, 1H), 7.93 (s, 1H), 7.62 (td, 1H), 7.37 (dd, 1H), 7.00 (dt, 1H), 6.28 (s, 1H), 5.01 (d, 1H), 4.80 (d, 1H), 3.14-3.08 (m, 3H).

### (1S)-1-(5,6-Difluoro-N-methyl-1H-indole-2-carboxamido)-8,9-difluoro-6-oxo-1,4,5,6-tetrahydro-2H-pyrano[3,4-c]isoquinolin-4-yl isobutyrate (Compound 300)

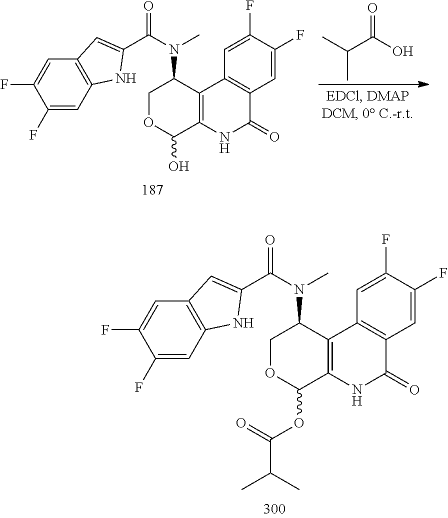

N-((1S)-8,9-Difluoro-4-hydroxy-6-oxo-1,4,5,6-tetrahydro-2H-pyrano[3,4-c]isoquinolin-1-yl)-5,6-difluoro-N-methyl-1H-indole-2-carboxamide (15 mg, 0.03 mmol), 2-methylpropanoic acid (6 mg, 0.07 mmol), and N,N-dimethylpyridin-4-amine (2 mg, 0.02 mmol) were combined and stirred in DCM (0.5 mL), at 0° C. 3-(Ethyliminomethyleneamino)-N,N-dimethyl-propan-1-amine hydrochloride (19 mg, 0.1 mmol) was added and the reaction mixture was allowed to warm to room temperature and stirred for 16 h. The reaction mixture was diluted with 10 mL of DCM and washed with 5% NaHCO₃(10 mL), then brine (10 mL), dried over sodium sulfate, filtered and the solvent was evaporated. The product was isolated by flash chromatography (silica gel, 15-100% EtOAc/hexanes) to afford (1S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate (12.8 mg, 74% yield). LCMS m/z found 532.1 [M+H]⁺, 444.3 [M−iPrCO₂]⁺, RT=8.10 min (Major); 8.16 min (minor), Major/minor=7:1 (Method A); ¹H NMR (400 MHz, CDCl₃) δ 11.92 (s, 1H), 11.66 (s, 1H)*, 10.00 (d, 1H), 8.23 (dt, 1H), 7.54 (dd, 1H), 7.46 (dd, 1H)*, 7.38 (dd, 1H), 7.34-7.24 (m, 1H), 6.90 (s, 1H), 6.86 (d, 1H), 6.79 (d, 1H)*, 6.12 (t, 1H)*, 5.99 (d, 1H), 4.53 (dd, 1H), 4.35 (dd, 1H)*, 4.28-4.17 (m, 1H), 3.27 (d, 3H), 2.84-2.74 (h, 1H)*, 2.67 (h, 1H), 1.30-1.25 (m, 6H)*, 1.23 (dd, 6H). NOTE. “*” indicates observable signals of minor isomer, assigned as the minor C-4 epimer.

### (1S)-1-(5,6-Difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate (Compound 331)

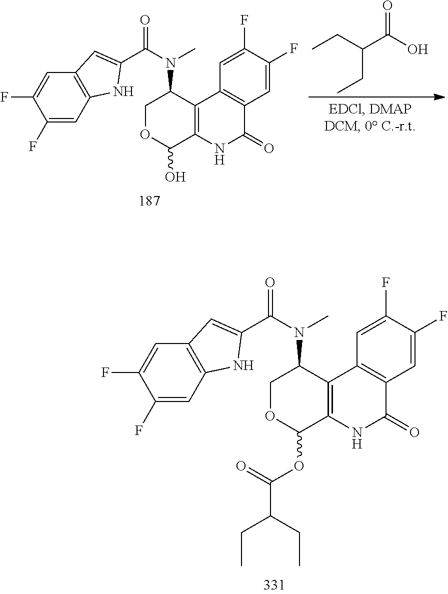

(1S)-1-(5,6-Difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate was synthesized in an analogous manner as described above from N-((1S)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide (187) and 2-ethylbutanoic acid. LCMS m/z found 560.2 [M+H]⁺, 444.1 [M−(3-Pent)CO₂]⁺, RT=5.66 min (Major, >95%) (Method A); ¹H NMR (400 MHz, CDCl₃) δ 11.40 (s, 1H), 9.95 (s, 1H), 8.24 (dd, 1H), 7.52 (dd, 1H), 7.38 (dd, 1H), 7.33-7.24 (m, 1H), 6.93 (s, 1H), 6.88-6.82 (m, 1H), 5.98 (d, 1H), 4.55 (dd, 1H), 4.25 (d, 1H), 3.26 (s, 3H), 2.34 (tt, 1H), 1.80-1.46 (m, 4H), 0.91 (ddd, 6H).

### N-((1S)-8,9-Difluoro-4-methoxy-6-oxo-1,4,5,6-tetrahydro-2H-pyrano[3,4-c]isoquinolin-1-yl)-5,6-difluoro-N-methyl-1H-indole-2-carboxamide (Compound 320)

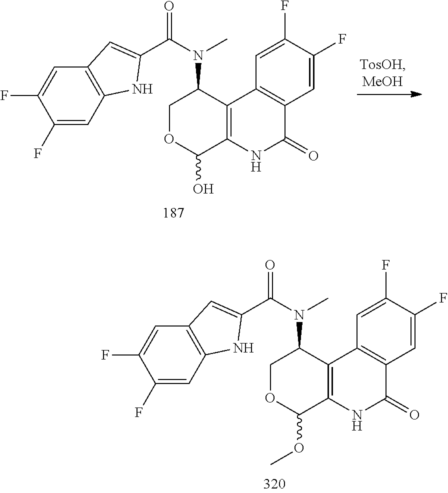 [SMILES: CN(C(=O)c1cc2cc(F)c(F)cc2[nH]1)[C@@H]1COC(O)c2[nH]c(=O)c3cc(F)c(F)cc3c21 ; COC1OC[C@@H](N(C)C(=O)c2cc3cc(F)c(F)cc3[nH]2)c2c1[nH]c(=O)c1cc(F)c(F)cc21]

N-((1S)-8,9-Difluoro-4-hydroxy-6-oxo-1,4,5,6-tetrahydro-2H-pyrano[3,4-c]isoquinolin-1-yl)-5,6-difluoro-N-methyl-1H-indole-2-carboxamide (15 mg, 0.03 mmol) and 4-methylbenzenesulfonic acid (6 mg, 0.03 mmol) were stirred in anhydrous methanol (1 mL) for 1 week. The solvent was evaporated, and the residue was loaded directly on a flash chromatography column. The product was isolated by flash chromatography (silica gel, 0-100% EtOAc/hexanes). LCMS m/z found 476.2 [M+H]⁺, RT=4.54 min (Method A); ¹H NMR (400 MHz, CDCl₃) δ 11.10 (s, 1H), 9.63 (d, 1H), 8.28-8.14 (m, 1H), 7.51 (dd, 1H), 7.38 (dt, 1H), 7.31-7.20 (m, 1H), 6.86 (d, 1H), 5.89 (d, 1H), 5.45 (s, 1H), 4.48 (dd, 1H), 4.17-4.06 (m, 1H), 3.66 (s, 3H), 3.27 (s, 3H).

### 5,6-Difluoro-N-methyl-N-((1S)-4,8,9-trifluoro-6-oxo-1,4,5,6-tetrahydro-2H-pyrano[3,4-c]isoquinolin-1-yl)-1H-indole-2-carboxamide (Compound 411)

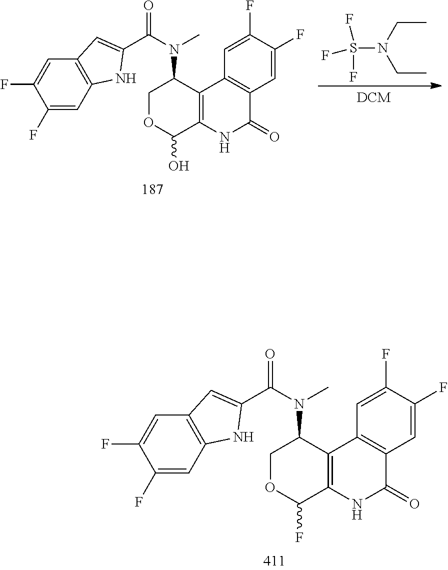

A stirred suspension of N-((1S)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide (24 mg, 0.05 mmol) in DCM (0.5 mL) at 0° C., was treated with N,N-diethyl-1,1,1-trifluoro-14-sulfanamine (DAST, 17 mg, 0.1 mmol), and then the reaction was allowed to warm to room temperature and stirred for an additional 1 h. The reaction mixture was diluted with 20 mL of DCM, washed with 10 mL of a 5% solution of sodium bicarbonate, and then with 10 mL of brine. The organic solution was dried on magnesium sulfate, filtered, and the solvent was evaporated under reduced pressure. The product was isolated by flash-chromatography (ISCO, silica gel, EtOAc/Hexanes 0-75%) to afford 5,6-difluoro-N-methyl-N-((1S)-4,8,9-trifluoro-6-oxo-1,4,5,6-tetrahydro-2H-pyrano[3,4-c]isoquinolin-1-yl)-1H-indole-2-carboxamide (8 mg, 33% yield) as a ˜7:1 mixture of epimers at the 4-position. LCMS m/z found 464.3 [M+H]⁺, RT(major)=4.50 min (Method A). ¹H NMR (major, 400 MHz, CDCl₃) δ 11.51 (s, 1H), 9.95 (s, 1H), 8.37-8.27 (m, 1H), 7.57 (dd, 1H), 7.38 (dd, 1H), 7.30 (dd, 1H), 6.88 (s, 1H), 6.34 (d, 1H), 6.02 (s, 1H), 4.61 (d, 1H), 4.43-4.27 (m, 1H), 3.26 (s, 3H).

### tert-Butyl-8-fluoro-1,6-dioxo-1,4,5,6-tetrahydrobenzo[c][1,7]naphthyridine-3(2H)-carboxylate (IVc)

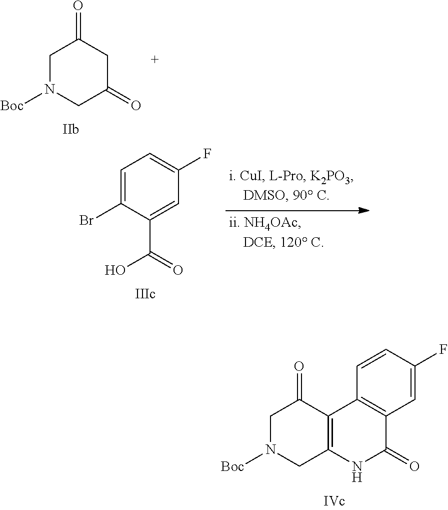 [SMILES: CC(C)(C)OC(=O)N1CC(=O)CC(=O)C1 ; O=C(O)c1cc(F)ccc1Br ; CC(C)(C)OC(=O)N1CC(=O)c2c([nH]c(=O)c3cc(F)ccc23)C1]

Step i: A mixture of 3.0 g (10.56 mmol, 1.0 eq.) of 4-fluoro-2-bromobenzoic acid (IIIc), 2.7 g (12.68, 1.2 eq.) of tert-butyl 3,5-dioxopiperidine-1-carboxylate (Ib), 5.8 g (42.2 mmol, 4.0 eq.) of potassium carbonate, 0.25 g (2.11 mmol, 0.2 eq.) of L-proline and 0.2 g (1.05 mmol, 0.1 eq.) of copper(I)iodide in 15 mL of dry DMSO under a nitrogen atmosphere was stirred for 16 h at 110° C. (Note: Reaction was performed on 3×3 g scale in parallel). On cooling to room temperature, the triplicate reaction mixtures were combined and diluted with cold water (100 mL). The mixture was then acidified with saturated citric acid solution (100 mL). The resulting suspension was filtered, and the filtrate was extracted with ethyl acetate (3×200 mL). The combined organic layer was washed with brine (100 mL), dried over anhydrous sodium sulfate, filtered and concentrated under reduced pressure to afford 12.2 g of a mixture of tert-butyl 8-fluoro-1,6-dioxo-1,2,4,6-tetrahydro-3H-isochromeno[3,4-c]pyridine-3-carboxylate and 2-(1-(tert-butoxycarbonyl)-5-hydroxy-3-oxo-1,2,3,6-tetrahydropyridin-4-yl)-4-fluorobenzoic acid which was taken into the next step without purification.

Step ii: To a mixture of 6 g of above prepared crude mixture of tert-butyl 8-fluoro-1,6-dioxo-1,2,4,6-tetrahydro-3H-isochromeno[3,4-c]pyridine-3-carboxylate and 2-(1-(tert-butoxycarbonyl)-5-hydroxy-3-oxo-1,2,3,6-tetrahydropyridin-4-yl)-4-fluorobenzoic acid in 30 mL of 1,2-dichlorethane in a seal tube was added 3.4 g (4.54 mmol, 2.5 eq.) of ammonium acetate and the mixture was heated at 120° C. for 16 h. (Note: Reaction was performed on 2×6 g scale in parallel). On cooling to room temperature, the duplicate reaction mixtures were combined, poured in ice-cold water (200 mL), and extracted with ethyl acetate (2×25 mL). The combined organic extracts were washed with brine (50 mL), dried over anhydrous sodium sulfate, filtered and concentrated under reduced pressure. The resulting crude product was triturated with acetone (50 mL) to afford 3.8 g (1.14 mmol, 28% over two steps) of tert-butyl 8-fluoro-1,6-dioxo-1,4,5,6-tetrahydrobenzo[c][1,7]naphthyridine-3(2H)-carboxylate (IVc) as a brown solid. LCMS: m/z found 331.47 [M−H]⁻; ¹H NMR (400 MHz, DMSO-d₆): δ 12.45 (br s, 1H), 9.03-8.97 (m, 1H), 8.12 (dd, 1H), 7.9 (dd, 1H), 4.71 (br s, 2H), 4.18 (br s, 2H), 1.42 (s, 9H).

### tert-Butyl 8-fluoro-1-(methylamino)-6-oxo-1,4,5,6-tetrahydrobenzo[c][1,7]naphthyridine-3(2H)-carboxylate (Vc)

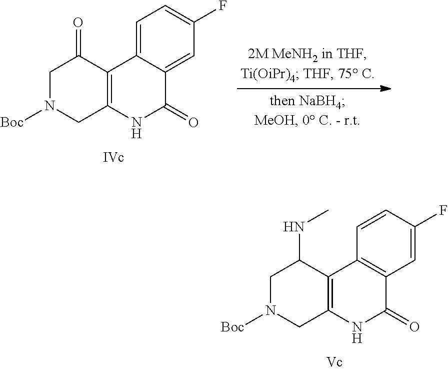 [SMILES: CC(C)(C)OC(=O)N1CC(=O)c2c([nH]c(=O)c3cc(F)ccc23)C1 ; CNC1CN(C(=O)OC(C)(C)C)Cc2[nH]c(=O)c3cc(F)ccc3c21]

To a stirred solution of 2.0 g (6.02 mmol, 1.0 eq.) of tert-butyl 8-fluoro-1,6-dioxo-1,4,5,6-tetrahydrobenzo[c][1,7]naphthyridine-3(2H)-carboxylate (IVc) in 10 mL of THE in sealed tube at room temperature under a nitrogen atmosphere was added 3.6 mL (7.2 mmol, 1.2 eq.) of a 2 M methylamine solution in THF followed by 10 mL (5 vol) of titanium isopropoxide and the reaction mixture was heated at 70° C. for 3 h. The mixture was allowed to cool to room temperature and further cooled to 0° C. and diluted with methanol (2 mL). To this mixture at 0° C., 0.69 mg (18.64 mmol, 3.0 eq.) of NaBH₄ was added portion-wise and then the reaction was continued at room temperature for 2 h. The mixture was then diluted with saturated brine (15 mL) and 10% MeOH in DCM (200 mL). After stirring for 30 min, the heterogeneous mixture was filtered and washed with 10% MeOH in DCM (50 mL). The filtrate was dried over anhydrous sodium sulfate and concentrated under reduced pressure. The resulting crude product was triturated with n-pentane (50 mL), the precipitated solid was collected by filtration and dried under vacuum to afford 1.3 g of tert-butyl 8-fluoro-1-(methylamino)-6-oxo-1,4,5,6-tetrahydrobenzo[c][1,7]naphthyridine-3(2H)-carboxylate (Vc). LCMS: m/z found 348.32 [M+H]⁺.

### N-(8-Fluoro-6-oxo-1,2,3,4,5,6-hexahydrobenzo[c][1,7]naphthyridin-1-yl)-N-methyl-1H-indole-2-carboxamide (Compounds 77 and 78)

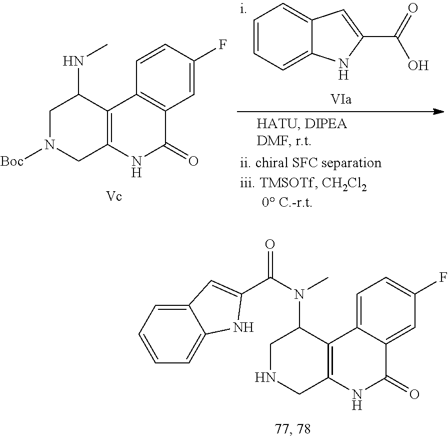

Step i: To a stirred solution of 0.15 g (0.93 mmol, 1.0 eq.) of 1H-indole-2-carboxylic acid (VIa) in 6 mL of DMF at room temperature, 0.45 mL (2.89 mmol, 3.0 eq.) of DIPEA, 0.49 g (1.29 mmol, 1. eq.) of HATU were added and the mixture was stirred for 10 min. To this mixture, 0.3 g (0.86 mmol, 1.0 eq.) of tert-butyl 8-fluoro-1-(methylamino)-6-oxo-1,4,5,6-tetrahydrobenzo[c][1,7]naphthyridine-3(2H)-carboxylate (Vc) was added and the resulting reaction mixture was stirred at room temperature for 16 h. The reaction mixture was then diluted with cold water (20 mL) and stirred for a further 30 min. The resulting solid was collected by filtration and washed with water (10 mL) to afford 0.3 g of (0.61 mmol, 70%) of tert-butyl 8-fluoro-1-(N-methyl-1H-indole-2-carboxamido)-6-oxo-1,4,5,6-tetrahydrobenzo[c][1,7]naphthyridine-3(2H)-carboxylate. LCMS: m/z found 489.51 [M+H]⁺.

Step ii: The enantiomers of tert-butyl 8-fluoro-1-(N-methyl-1H-indole-2-carboxamido)-6-oxo-1,4,5,6-tetrahydrobenzo[c][1,7]naphthyridine-3(2H)-carboxylate were subsequently separated by chiral preparative SFC: method isocratic, mobile phase methanol:CO₂—40:60. Column: Chiralcel OJ (21×250 mm), 5μ, flow rate: 70 g/min.

Step iii: Each individual enantiomer of tert-butyl 8-fluoro-1-(N-methyl-1H-indole-2-carboxamido)-6-oxo-1,4,5,6-tetrahydrobenzo[c][1,7]naphthyridine-3(2H)-carboxylate isolated as described above was converted to the final product in an analogous manner as described above for Compounds 3 and 4 (Step ii) and purified by preparative reverse phase hplc: Column/dimensions: X-Bridge C18 (19×150 mm) 5 μm, Mobile phase A: 10 mM Ammonium bicarbonate in water; Mobile phase B: Acetonitrile, Gradient, Flow rate: 15 ml/min.

Enantiomer I (Compound 77): LCMS: m/z found 391.2 [M+H]⁺, RT=3.31 min, (Method A); ¹H NMR (400 MHz, DMSO-d₆ δ 11.80 (s, 2H), 11.55 (s, 1H), 7.89 (m, 1H), 7.60 (m, 2H), 7.46 (m, 2H), 7.20 (m, 1H), 7.03 (m, 1H), 6.86 (s, 1H), 5.69 (br s, 1H), 3.80 (d, 1H), 3.67 (d, 1H), 3.17 (m, 2H), 3.14 (s, 3H); Chiral analytical SFC: RT=6.04 min, Column: Chiralpak IC-3 (4.6×150 mm) 3 μm, 40% (0.2% 7 M Methanolic ammonia in ACN:MeOH (1:1)), Flow rate: 3.0 g/min.

Enantiomer II (Compound 78): LCMS: m/z found 391.2 [M+H]⁺, RT=3.31 min, (Method A); ¹H NMR (400 MHz, DMSO-d₆) δ 11.80 (s, 2H), 11.55 (s, 1H), 7.89 (m, 1H), 7.60 (m, 2H), 7.46 (m, 2H), 7.20 (m, 1H), 7.03 (m, 1H), 6.86 (s, 1H), 5.69 (br s, 1H), 3.80 (d, 1H), 3.67 (d, 1H), 3.17 (m, 2H), 3.14 (s, 3H); Chiral analytical SFC: RT=8.05 min, Column: Chiralpak IC-3 (4.6×150 mm) 3 μm, 40% (0.2% 7 M Methanolic ammonia in ACN:MeOH (1:1)), Flow rate: 3.0 g/min.

### 4-Bromo-3,5-difluoro-N-(8-fluoro-6-oxo-1,2,3,4,5,6-hexahydrobenzo[c][1,7]naphthyridin-1-yl)-N-methylbenzamide (Compounds 170 and 171)

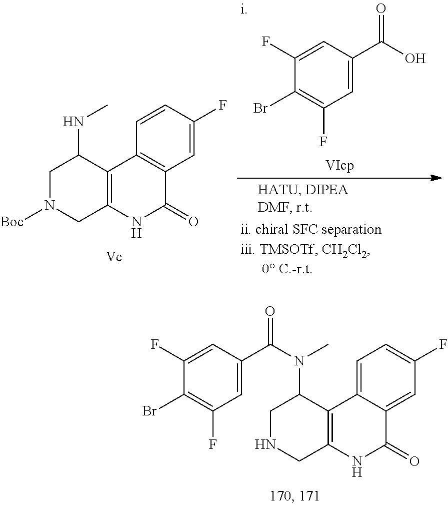

Individual enantiomers of 4-bromo-3,5-difluoro-N-(8-fluoro-6-oxo-1,2,3,4,5,6-hexahydrobenzo[c][1,7]naphthyridin-1-yl)-N-methylbenzamide were prepared in an analogous manner as described above (for Compounds 77, 78), from racemic tert-butyl 8-fluoro-1-(methylamino)-6-oxo-1,4,5,6-tetrahydrobenzo[c][1,7]naphthyridine-3(2H)-carboxylate (Vc) and 4-bromo-3,5-difluorobenzoic acid (VIcp).

Enantiomer I (Compound 170): LCMS: m/z found 466.1 [M+H]⁺, RT=4.86 min, (Method A); ¹H NMR (400 MHz, DMSO-d₆) δ 11.52 (s, 1H), 7.89-7.86 (m, 1H), 7.75-7.70 (m, 1H), 7.53-7.49 (m, 1H), 7.38-7.36 (d, 2H), 5.56 (s, 1H), 3.75 (d, 1H), 3.63 (d, 1H), 3.32-3.25 (m, 1H), 3.12-3.08 (m, 1H), 2.76-2.71 (m, 1H), 2.66 (s, 3H); Chiral analytical SFC: RT=3.75 min, Column: Chiralcel OX-3 (4.6×150 mm) 3 μm, 30% (0.5% DEA in Methanol), Flow rate: 3.0 g/min.

Enantiomer II (Compound 171): LCMS: m/z found 466.1 [M+H]⁺, RT=4.86 min, (Method A); ¹H NMR (400 MHz, DMSO-d₆) δ 11.52 (s, 1H), 7.89-7.86 (m, 1H), 7.75-7.70 (m, 1H), 7.53-7.49 (m, 1H), 7.38-7.36 (d, 2H), 5.56 (s, 1H), 3.75 (d, 1H), 3.63 (d, 1H), 3.32-3.25 (m, 1H), 3.12-3.08 (m, 1H), 2.76-2.71 (m, 1H), 2.66 (s, 3H); Chiral analytical SFC: RT=4.74 min, Column: Chiralcel OX-3 (4.6×150 mm) 3 μm, 30% (0.5% DEA in Methanol), Flow rate: 3.0 g/min.

### 4-Chloro-3,5-difluoro-N-(8-fluoro-6-oxo-1,2,3,4,5,6-hexahydrobenzo[c][1,7]naphthyridin-1-yl)-N-methylbenzamide (Compounds 176 and 177)

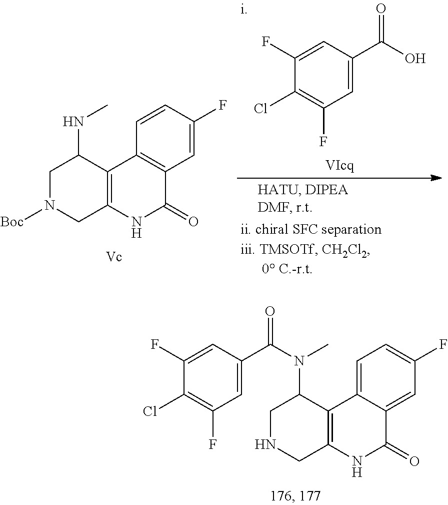

Individual enantiomers of 4-chloro-3,5-difluoro-N-(8-fluoro-6-oxo-1,2,3,4,5,6-hexahydrobenzo[c][1,7]naphthyridin-1-yl)-N-methylbenzamide were prepared in an analogous manner as described above (for Compounds 77, 78), from racemic tert-butyl 8-fluoro-1-(methylamino)-6-oxo-1,4,5,6-tetrahydrobenzo[c][1,7]naphthyridine-3(2H)-carboxylate (Vc) and 4-chloro-3,5-difluorobenzoic acid (VIcq).

Enantiomer I (Compound 176): LCMS: m/z found 422.2 [M+H]⁺, RT=3.67 min, (Method A); ¹H NMR (400 MHz, DMSO-d₆) δ 11.52 (s, 1H), 7.89-7.86 (dd, 1H), 7.75-7.70 (m, 1H), 7.53-7.49 (m, 1H), 7.43 (d, 2H), 5.56 (s, 1H), 3.75 (d, 1H), 3.63 (d, 1H), 3.26 (s, 1H), 3.12-3.08 (m, 1H), 2.67 (m, 4H); Chiral analytical SFC: RT=3.11 min, Column: Chiralcel OX-3 (4.6×150 mm) 3 μm, 30% (0.5% DEA in Methanol), Flow rate: 3.0 g/min.

Enantiomer II (Compound 177): LCMS: m/z found 422.2 [M+H]⁺, RT=3.67 min, (Method A); ¹H NMR (400 MHz, DMSO-d₆) δ 11.52 (s, 1H), 7.89-7.86 (dd, 1H), 7.75-7.70 (m, 1H), 7.53-7.49 (m, 1H), 7.43 (d, 2H), 5.56 (s, 1H), 3.75 (d, 1H), 3.63 (d, 1H), 3.26 (s, 1H), 3.12-3.08 (m, 1H), 2.67 (m, 4H); Chiral analytical SFC: RT=3.84 min, Column: Chiralcel OX-3 (4.6×150 mm) 3 μm, 30% (0.5% DEA in Methanol), Flow rate: 3.0 g/min.

### 4-(Difluoromethyl)-3,5-difluoro-N-(8-fluoro-6-oxo-1,2,3,4,5,6-hexahydrobenzo[c][1,7]naphthyridin-1-yl)-N-methylbenzamide (Compounds 185 and 186)

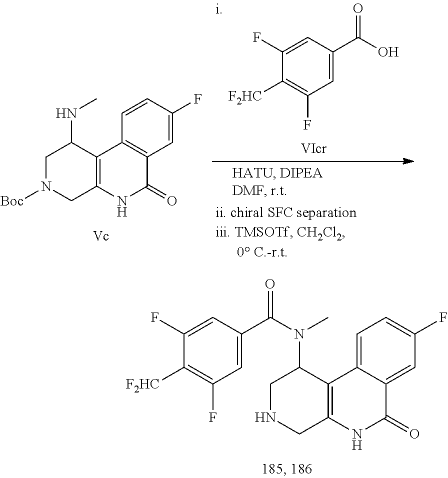 [SMILES: CNC1CN(C(=O)OC(C)(C)C)Cc2[nH]c(=O)c3cc(F)ccc3c21 ; O=C(O)c1cc(F)c(C(F)F)c(F)c1 ; CN(C(=O)c1cc(F)c(C(F)F)c(F)c1)C1CNCc2[nH]c(=O)c3cc(F)ccc3c21]

Individual enantiomers of 4-(difluoromethyl)-3,5-difluoro-N-(8-fluoro-6-oxo-1,2,3,4,5,6-hexahydrobenzo[c][1,7]naphthyridin-1-yl)-N-methylbenzamide were prepared in an analogous manner as described above (for Compounds 77, 78), from racemic tert-butyl 8-fluoro-1-(methylamino)-6-oxo-1,4,5,6-tetrahydrobenzo[c][1,7]naphthyridine-3(2H)-carboxylate (Vc) and 4-(difluoromethyl)-3,5-difluorobenzoic acid (VIcr).

Enantiomer I (Compound 185): LCMS: m/z found 438.3 [M+H]⁺, RT=6.40 min, (Method A); ¹H NMR (400 MHz, DMSO-d₆) δ 11.52 (s, 1H), 7.89-7.86 (dd, 1H), 7.76-7.67 (m, 1H), 7.59-7.49 (m, 1H), 7.45-7.19 (m, 3H), 5.56 (br s, 1H), 3.75 (d, 1H), 3.64 (d, 1H), 3.27 (m, 1H), 3.10 (m, 1H), 2.75 (m, 1H), 2.66 (s, 3H); Chiral analytical SFC: RT=2.03 min, Column: Chiralcel OX-3 (4.6×150 mm) 3 μm, 30% (0.5% DEA in Methanol), Flow rate: 3.0 g/min.

Enantiomer II (Compound 186): LCMS: m/z found 438.3 [M+H]⁺, RT=6.40 min, (Method A); ¹H NMR (400 MHz, DMSO-d₆) δ 11.52 (s, 1H), 7.89-7.86 (dd, 1H), 7.76-7.67 (m, 1H), 7.59-7.49 (m, 1H), 7.45-7.19 (m, 3H), 5.56 (br s, 1H), 3.75 (d, 1H), 3.64 (d, 1H), 3.27 (m, 1H), 3.10 (m, 1H), 2.75 (m, 1H), 2.66 (s, 3H); Chiral analytical SFC: RT=2.54 min, Column: Chiralcel OX-3 (4.6×150 mm) 3 μm, 30% (0.5% DEA in Methanol), Flow rate: 3.0 g/min.

### N-(8-Fluoro-6-oxo-1,2,3,4,5,6-hexahydrobenzo[c][1,7]naphthyridin-1-yl)-4-(difluoromethyl)-N-methyl-1H-indole-2-carboxamide (Compounds 209 and 210)

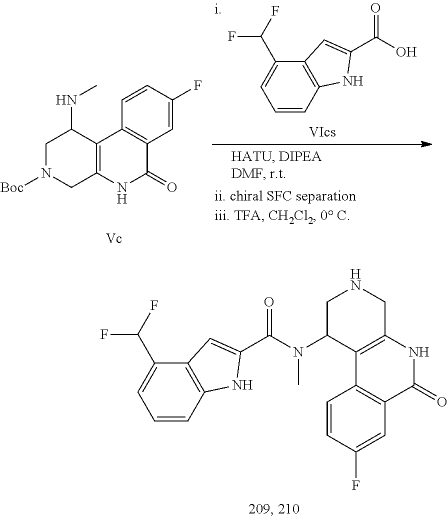 [SMILES: CNC1CN(C(=O)OC(C)(C)C)Cc2[nH]c(=O)c3cc(F)ccc3c21 ; O=C(O)c1cc2c(C(F)F)cccc2[nH]1 ; CN(C(=O)c1cc2c(C(F)F)cccc2[nH]1)C1CNCc2[nH]c(=O)c3cc(F)ccc3c21]

Individual enantiomers of N-(8-fluoro-6-oxo-1,2,3,4,5,6-hexahydrobenzo[c][1,7]naphthyridin-1-yl)-4-(difluoromethyl)-N-methyl-1H-indole-2-carboxamide were prepared in an analogous manner as described above (for Compounds 77, 78), from racemic tert-butyl 8-fluoro-1-(methylamino)-6-oxo-1,4,5,6-tetrahydrobenzo[c][1,7]naphthyridine-3(2H)-carboxylate (Vc) and 4-(difluoromethyl)-1H-indole-2-carboxylic acid (VIcs), except that separated isomers of intermediate tert-butyl 1-(4-(difluoromethyl)-N-methyl-1H-indole-2-carboxamido)-8-fluoro-6-oxo-1,4,5,6-tetrahydrobenzo[c][1,7]naphthyridine-3(2H)-carboxylate were treated with TFA (5.0 eq.) in DCM at 0° C., instead of TMSOTf, for the final, deprotection step.

Enantiomer I (Compound 209): LCMS: m/z found 441.3 [M+H]⁺, RT=4.51 min, (Method A); ¹H NMR (400 MHz, DMSO-d₆) δ 12.04 (bs, 1H), 11.56 (bs, 1H), 7.99-7.88 (d, 1H), 7.64-7.60 (m, 2H), 7.48-7.27 (m, 4H), 6.96 (s, 1H), 5.69 (s, 1H), 3.82 (d, 1H), 3.69 (d, 1H), 3.32-3.12 (m, 6H); Chiral analytical SFC: RT=2.90 min, Column: Chiralcel OJ-3 (4.6×150 mm) 3 μm, 20% (0.5% DEA in Methanol), Flow rate: 3.0 g/min.

Enantiomer II (Compound 210): LCMS: m/z found 441.3 [M+H]⁺, RT=4.51 min, (Method A); ¹H NMR (400 MHz, DMSO-d₆) δ 12.04 (bs, 1H), 11.56 (bs, 1H), 7.99-7.88 (d, 1H), 7.64-7.60 (m, 2H), 7.48-7.27 (m, 4H), 6.96 (s, 1H), 5.69 (s, 1H), 3.82 (d, 1H), 3.69 (d, 1H), 3.32-3.12 (m, 6H); Chiral analytical SFC: RT=4.42 min, Column: Chiralcel OJ-3 (4.6×150 mm) 3 μm, 20% (0.5% DEA in Methanol), Flow rate: 3.0 g/min.

### 4-(Difluoromethyl)-6-fluoro-N-(8-fluoro-6-oxo-1,2,3,4,5,6-hexahydrobenzo[c][1,7]naphthyridin-1-yl)-N-methyl-1H-indole-2-carboxamide (Compounds 259 and 260)

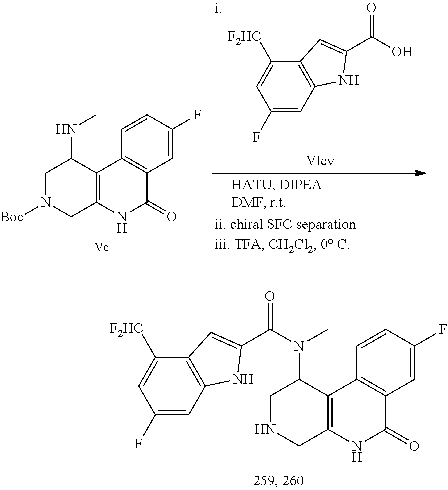 [SMILES: CNC1CN(C(=O)OC(C)(C)C)Cc2[nH]c(=O)c3cc(F)ccc3c21 ; O=C(O)c1cc2c(C(F)F)cc(F)cc2[nH]1 ; CN(C(=O)c1cc2c(C(F)F)cc(F)cc2[nH]1)C1CNCc2[nH]c(=O)c3cc(F)ccc3c21]

Individual enantiomers of 4-(difluoromethyl)-6-fluoro-N-(8-fluoro-6-oxo-1,2,3,4,5,6-hexahydrobenzo[c][1,7]naphthyridin-1-yl)-N-methyl-1H-indole-2-carboxamide were prepared in an analogous manner as described above (for Compounds 209, 210), from racemic tert-butyl 8-fluoro-1-(methylamino)-6-oxo-1,4,5,6-tetrahydrobenzo[c][1,7]naphthyridine-3(2H)-carboxylate (Vc) and 4-(difluoromethyl)-6-fluoro-1H-indole-2-carboxylic acid (VIcv).

Enantiomer I (Compound 259): LCMS: m/z found 459.4 [M+H]⁺, RT=6.19 min, (Method A); ¹H NMR (400 MHz, DMSO-d₆) δ 12.12 (bs, 1H), 11.60 (bs, 1H), 7.9 (d, 1H), 7.62 (t, 1H), 7.46-7.18 (m, 4H), 6.99 (s, 1H), 5.68 (s, 1H), 3.80 (d, 1H), 3.66 (d, 1H), 3.16-3.13 (m, 4H), 2.91-2.76 (m, 2H); Chiral analytical SFC: RT=1.22 min, Column: Chiralcel OJ-3 (4.6×150 mm) 3 μm, 30% (0.5% DEA in Methanol), Flow rate: 3.0 g/min.

Enantiomer II (Compound 260): LCMS: m/z found 459.4 [M+H]⁺, RT=6.19 min, (Method A); ¹H NMR (400 MHz, DMSO-d₆) δ 12.12 (bs, 1H), 11.60 (bs, 1H), 7.9 (d, 1H), 7.62 (t, 1H), 7.46-7.18 (m, 4H), 6.99 (s, 1H), 5.68 (s, 1H), 3.80 (d, 1H), 3.66 (d, 1H), 3.16-3.13 (m, 4H), 2.91-2.76 (m, 2H); Chiral analytical SFC: RT=1.65 min, Column: Chiralcel OJ-3 (4.6×150 mm) 3 μm, 30% (0.5% DEA in Methanol), Flow rate: 3.0 g/min.

### 4-(Difluoromethyl)-5-fluoro-N-(8-fluoro-6-oxo-1,2,3,4,5,6-hexahydrobenzo[c][1,7]naphthyridin-1-yl)-N-methyl-1H-indole-2-carboxamide (Compounds 271 and 272)

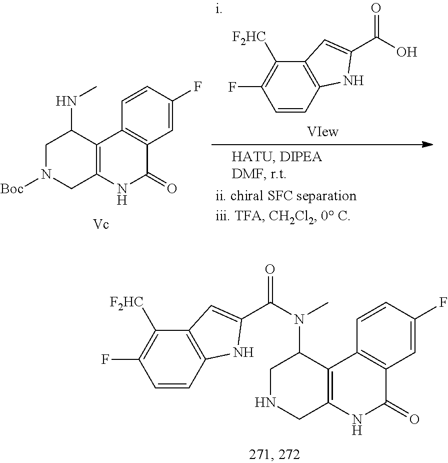

Individual enantiomers of 4-(difluoromethyl)-5-fluoro-N-(8-fluoro-6-oxo-1,2,3,4,5,6-hexahydrobenzo[c][1,7]naphthyridin-1-yl)-N-methyl-1H-indole-2-carboxamide were prepared in an analogous manner as described above (for Compounds 209, 210), from racemic tert-butyl 8-fluoro-1-(methylamino)-6-oxo-1,4,5,6-tetrahydrobenzo[c][1,7]naphthyridine-3(2H)-carboxylate (Vc) and 4-(difluoromethyl)-5-fluoro-1H-indole-2-carboxylic acid (VIcw).

Enantiomer I (Compound 271): LCMS: m/z found 459.3 [M+H]⁺, RT=6.78 min, (Method A); ¹H NMR (400 MHz, DMSO-d₆) δ 12.15 (bs, 1H), 11.57 (bs, 1H), 7.90-7.87 (m, 1H), 7.65-7.31 (m, 4H), 7.21-7.16 (m, 1H), 6.95 (d, 1H), 5.67 (s, 1H), 3.82-3.64 (m, 2H), 3.24-2.84 (m, 6H); Chiral analytical SFC: RT=1.86 min, Column: Chiralcel AS-3 (4.6×150 mm) 3 μm, 30% (0.5% DEA in Methanol), Flow rate: 3.0 g/min.

Enantiomer II (Compound 272): LCMS: m/z found 459.3 [M+H]⁺, RT=6.78 min, (Method A); ¹H NMR (400 MHz, DMSO-d₆) δ 12.15 (bs, 1H), 11.57 (bs, 1H), 7.90-7.87 (m, 1H), 7.65-7.31 (m, 4H), 7.21-7.16 (m, 1H), 6.95 (d, 1H), 5.67 (s, 1H), 3.82-3.64 (m, 2H), 3.24-2.84 (m, 6H); Chiral analytical SFC: RT=3.24 min, Column: Chiralcel AS-3 (4.6×150 mm) 3 μm, 30% (0.5% DEA in Methanol), Flow rate: 3.0 g/min.

### 3-(2-((tert-Butyldimethylsilyl)oxy)ethyl)-8-fluoro-3,4-dihydrobenzo[c][1,7]naphthyridine-1,6(2H,5H)-dione (IVd)

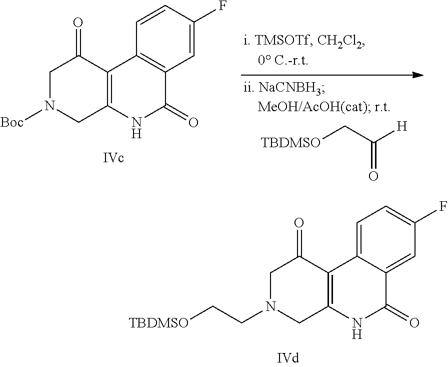

Step i: To a stirred solution of 2.0 g (6.024 mmol, 1.0 eq.) of tert-butyl 8-fluoro-1,6-dioxo-1,4,5,6-tetrahydrobenzo[c][1,7]naphthyridine-3(2H)-carboxylate (IVc) in 20 mL of DCM at 0° C. was added 1.6 mL (9.03 mmol, 1.5 eq.) of trimethylsilyl trifluoromethanesulfonate and the resulting reaction mixture was stirred at room temperature for 1 h. The volatiles were removed under reduced pressure and the residue was triturated with saturated sodium bicarbonate solution (20 mL). The solids were collected by filtration and dried under vacuum to afford 1.3 g (5.85 mmol, 93%) of 8-fluoro-3,4-dihydrobenzo[c][1,7]naphthyridine-1,6-(2H,5H)-dione as a brown solid. LCMS: m/z found 233.36 [M−H]⁻.

Step ii: To a stirred solution of 1.75 g (7.54 mmol, 1.0 eq.) of 8-fluoro-3,4-dihydrobenzo[c][1,7]naphthyridine-1,6(2H,5H)-dione in 17.5 mL of methanol, 1.96 g (11.31, 1.5 eq.) of 2-((tert-butyldimethylsilyl)oxy)acetaldehyde, 0.87 mL of acetic acid and 0.95 g (15.08 mmol, 2.0 eq.) of sodium cyanoborohydride were added and the resulting reaction mixture was stirred at room temperature for 16 h. The reaction mixture was then concentrated, and the residue was diluted with water (50 mL) and stirred for 30 min. The precipitated solid was collected by filtration and dried under vacuum to afford 1.3 g (3.3 mmol, 45%) of 3-(2-((tert-butyldimethylsilyl)oxy)ethyl)-8-fluoro-3,4-dihydrobenzo[c][1,7]naphthyridine-1,6(2H,5H)-dione (IVd). LCMS: m/z found 391.17 [M+H]⁻.

### 3-(2-((tert-Butyldimethylsilyl)oxy)ethyl)-8-fluoro-1-(methylamino)-1,3,4,5-tetrahydrobenzo[c][1,7]naphthyridin-6(2H)-one (Vd)

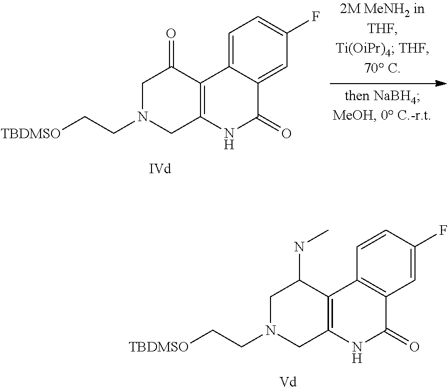 [SMILES: CC(C)(C)[Si](C)(C)OCCN1CC(=O)c2c([nH]c(=O)c3cc(F)ccc23)C1 ; CNC1CN(CCO[Si](C)(C)C(C)(C)C)Cc2[nH]c(=O)c3cc(F)ccc3c21]

3-(2-((tert-Butyldimethylsilyl)oxy)ethyl)-8-fluoro-1-(methylamino)-1,3,4,5-tetrahydrobenzo[c][1,7]naphthyridin-6(2H)-one was prepared from methylamine and 3-(2-((tert-butyldimethylsilyl)oxy)ethyl)-8-fluoro-3,4-dihydrobenzo[c][1,7]naphthyridine-1,6(2H,5H)-dione (IVd) by a procedure similar to the one described above for Vc. LCMS: m/z found 406.5 [M−H]⁻.

### N-(8-Fluoro-3-(2-hydroxyethyl)-6-oxo-1,2,3,4,5,6-hexahydrobenzo[c][1,7]naphthyridin-1-yl)-N-methyl-1H-indole-2-carboxamide (Compounds 79 and 80)

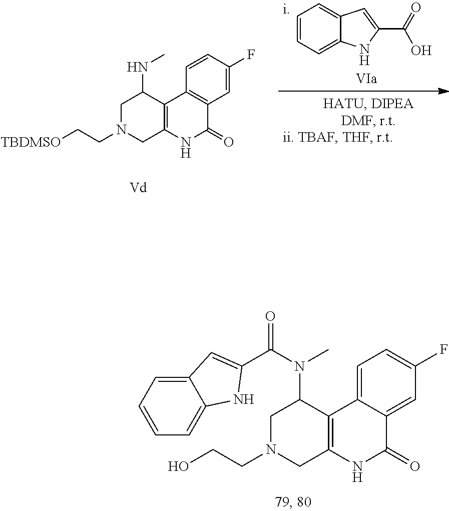 [SMILES: O=C(O)c1cc2ccccc2[nH]1 ; CNC1CN(CCO[Si](C)(C)C(C)(C)C)Cc2[nH]c(=O)c3cc(F)ccc3c21 ; CN(C(=O)c1cc2ccccc2[nH]1)C1CN(CCO)Cc2[nH]c(=O)c3cc(F)ccc3c21]

Step i: 1H-Indole-2-carboxylic acid (VIa) and 3-(2-((tert-butyldimethylsilyl)oxy)ethyl)-8-fluoro-1-(methylamino)-1,3,4,5-tetrahydrobenzo[c][1,7]naphthyridin-6(2H)-one (Vd) were converted to N-(8-fluoro-3-(2-(hydroxy-t)ethyl)-6-oxo-1,2,3,4,5,6-hexahydrobenzo[c][1,7]naphthyridin-1-yl)-N-methyl-1H-indole-2-carboxamide by a procedure similar to the one described above. LCMS: m/z found 423.51 [M+H]⁺.

Step ii: To a stirred solution of 0.35 g (0.63 mmol, 1.0 eq.) of N-(8-fluoro-3-(2-(hydroxy-t)ethyl)-6-oxo-1,2,3,4,5,6-hexahydrobenzo[c][1,7]naphthyridin-1-yl)-N-methyl-1H-indole-2-carboxamide in 7 mL of THF at 0° C., 0.95 mL (0.95 mmol, 1.5 eq.) of TBAF was added and the reaction mixture was stirred at room temperature for 2 h. The reaction was then quenched with MeOH (1 mL) and then organic volatiles were evaporated under reduced pressure. The residue was diluted with water (20 mL) and extracted with ethyl acetate (2×30 mL). The combined organic layer was washed with brine (20 mL), dried over sodium sulfate, and concentrated to afford 0.24 g (0.40 mmol 85%) of N-(8-fluoro-3-(2-hydroxyethyl)-6-oxo-1,2,3,4,5,6-hexahydrobenzo[c][1,7]naphthyridin-1-yl)-N-methyl-1H-indole-2-carboxamide as an off-white solid, which was further purified by reverse phase HPLC. The enantiomers were subsequently separated by preparative SFC: Method isocratic, Mobile phase methanol:CO₂—40:60. Column: DCPAK P4VP (21×250 mm), 5 μm, flow rate: 70 g/min.

Enantiomer I (Compound 79): LCMS: m/z found 435.3 [M+H]⁺, RT=3.29 min, (Method A); ¹H NMR (400 MHz, DMSO-d₆) δ 11.66 (m, 2H), 7.90-7.80 (m, 1H), 7.60-7.50 (m, 2H), 7.46-7.40 (m, 2H) 7.21 (t, 1H), 7.05 (m, 1H), 6.85 (s, 1H), 5.83 (s, 1H), 4.53 (s, 1H), 3.88 (d, 1H), 3.53-3.58 (m, 2H), 3.15 (s, 3H), 3.02 (d, 2H), 2.83 (m, 1H), 2.67 (m, 2H); Chiral analytical SFC: RT=4.08 min, Column: Chiralpak IC-3 (4.6×150 mm) 3 μm, 40% of Methanol, Flow rate: 3.0 g/min.

Enantiomer II (Compound 80): LCMS: m/z found 435.2 [M+H]⁺, RT=3.29 min, (Method A); ¹H NMR (400 MHz, DMSO-d₆) δ 11.66 (m, 2H), 7.90-7.80 (m, 1H), 7.60-7.50 (m, 2H), 7.46-7.40 (m, 2H) 7.21 (t, 1H), 7.05 (m, 1H), 6.85 (s, 1H), 5.83 (s, 1H), 4.53 (s, 1H), 3.88 (d, 1H), 3.53-3.58 (m, 2H), 3.15 (s, 3H), 3.02 (d, 2H), 2.83 (m, 1H), 2.67 (m, 2H); Chiral analytical SFC: RT=6.49 min, Column: Chiralpak IC-3 (4.6×150 mm) 3 μm, 40% of Methanol, Flow rate: 3.0 g/min.

### 8-Fluoro-3-methyl-3,4-dihydrobenzo[c][1,7]naphthyridine-1,6(2H,5H)-dione (IVe)

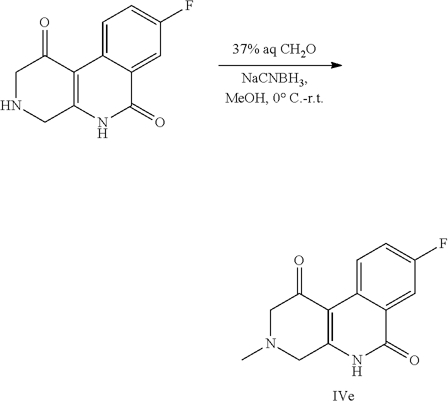 [SMILES: O=C1CNCc2[nH]c(=O)c3cc(F)ccc3c21 ; CN1CC(=O)c2c([nH]c(=O)c3cc(F)ccc23)C1]

To a stirred solution of 1.0 g (4.31 mmol, 1.0 eq.) of 8,9-difluoro-3,4-dihydrobenzo[c][1,7]naphthyridine-1,6(2H,5H)-dione (obtained as described above for IVd, Step i) in 10 mL of methanol were added 5 mL of 37% aqueous solution of formaldehyde and 0.54 g (8.62 mmol, 2.0 eq.) of sodium cyanoborohydride and the resulting mixture was stirred at room temperature for 16 h. The mixture was then diluted with water (150 mL) and extracted with ethyl acetate (3×150 mL). The combined organic extracts were dried over anhydrous sodium sulfate and concentrated under reduced pressure to afford crude 0.75 g (3.17 mmol, 78%) of 8-fluoro-3-methyl-3,4-dihydrobenzo[c][1,7]naphthyridine-1,6(2H,5H)-dione (IVe). LCMS: m/z found 247.19 [M+H]⁺.

### 8-Fluoro-3-methyl-1-(methylamino)-1,3,4,5-tetrahydrobenzo[c][1,7]naphthyridin-6(2H)-one (Ve)

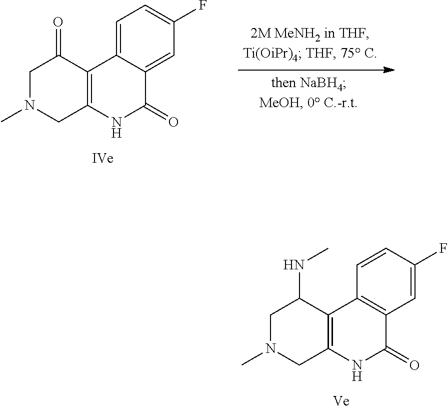 [SMILES: CN1CC(=O)c2c([nH]c(=O)c3cc(F)ccc23)C1 ; CNC1CN(C)Cc2[nH]c(=O)c3cc(F)ccc3c21]

Racemic 8-fluoro-3-methyl-1-(methylamino)-1,3,4,5-tetrahydrobenzo[c][1,7]naphthyridin-6(2H)-one was synthesized in an analogous manner as described above for Vd, from 8-fluoro-3-methyl-3,4-dihydrobenzo[c][1,7]naphthyridine-1,6(2H,5H)-dione (IVe) and methylamine. LCMS: m/z found 262.29 [M+H]⁺.

### N-(8-Fluoro-3-methyl-6-oxo-1,2,3,4,5,6-hexahydrobenzo[c][1,7]naphthyridin-1-yl)-N-methyl-1H-indole-2-carboxamide (Compounds 60 and 61)

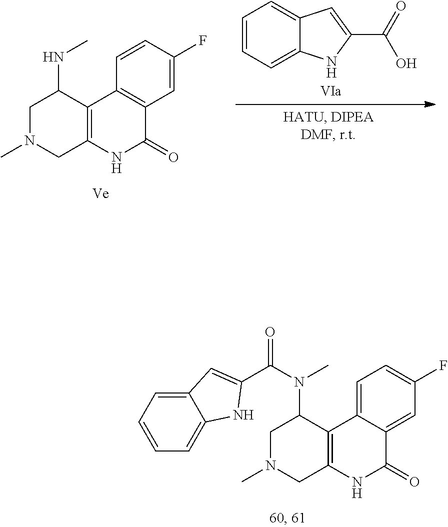

Racemic N-(8-fluoro-3-methyl-6-oxo-1,2,3,4,5,6-hexahydrobenzo[c][1,7]naphthyridin-1-yl)-N-methyl-1H-indole-2-carboxamide was synthesized in an analogous manner as described above from racemic 8-fluoro-3-methyl-1-(methylamino)-1,3,4,5-tetrahydrobenzo[c][1,7]naphthyridin-6(2H)-one (Ve) and 1H-indole-2-carboxylic acid (VIa). The enantiomers were subsequently separated by preparative SFC: Method isocratic, Mobile phase methanol:CO₂—40:60. Column: Chiralpak IC (30×250 mm), 5 μm, flow rate: 100 g/min.

Enantiomer I (Compound 60): LCMS: m/z found 405.2 [M+H]⁺, RT=2.99 min, (Method A); ¹H NMR (400 MHz, DMSO-d₆) δ 11.66 (br s, 2H), 7.90-7.87 (m, 1H) 7.63-7.57 (m, 2H), 7.47-7.42 (m, 2H), 7.20 (t, 1H), 7.07 (t, 1H), 6.85 (s, 1H), 5.82 (s, 1H), 3.72 (d, 1H), 3.14-3.01 (m, 5H), 2.73-2.69 (m, 1H), 2.35 (s, 3H); Chiral analytical SFC: RT=3.59 min, Column: Chiralpak IC-3 (4.6×150 mm) 3 μm, 40% of (0.5% DEA in MeOH), Flow rate: 3.0 g/min.

Enantiomer II (Compound 61): LCMS: m/z found 405.2 [M+H]⁺, RT=2.99 min, (Method A); ¹H NMR (400 MHz, DMSO-d₆) δ 11.66 (br s, 2H), 7.90-7.87 (m, 1H) 7.63-7.57 (m, 2H), 7.47-7.42 (m, 2H), 7.20 (t, 1H), 7.07 (t, 1H), 6.85 (s, 1H), 5.82 (s, 1H), 3.72 (d, 1H), 3.14-3.01 (m, 5H), 2.73-2.69 (m, 1H), 2.35 (s, 3H); Chiral analytical SFC: RT=5.71 min, Column: Chiralpak IC-3 (4.6×150 mm) 3 μm, 40% of (0.5% DEA in MeOH), Flow rate: 3.0 g/min.

### 3-Acetyl-8-fluoro-3,4-dihydrobenzo[c][1,7]naphthyridine-1,6(2H,5H)-dione (IVf)

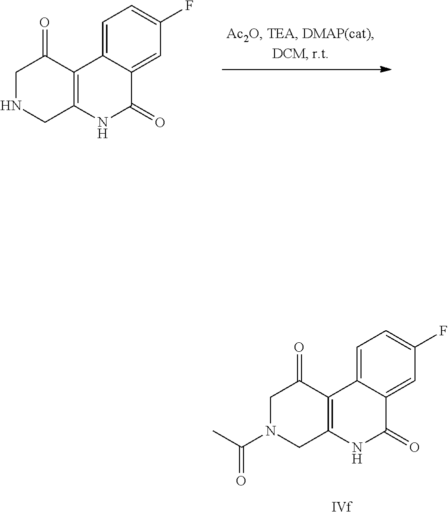 [SMILES: O=C1CNCc2[nH]c(=O)c3cc(F)ccc3c21 ; CC(=O)N1CC(=O)c2c([nH]c(=O)c3cc(F)ccc23)C1]

To a stirred solution of 0.5 g (2.16 mmol, 3.0 eq.) of fluoro-3,4-dihydrobenzo[c][1,7]naphthyridine-1,6(2H,5H)-dione (obtained as described above for IVd, Step i) in 5 mL of dichloromethane, were added 0.6 mL (4.31 mmol, 2.0 eq.) of triethylamine and 0.20 mL (2.16 mmol, 1.0 eq.) of acetic anhydride and the mixture was stirred at room temperature for 4 h. The reaction mixture was concentrated and washed with water (20 mL) to afford 0.4 g of 3-acetyl-8-fluoro-3,4-dihydrobenzo[c][1,7]naphthyridine-1,6(2H,5H)-dione (IVf) as a pale yellow solid. LCMS: m/z found 275.25 [M+H]⁺; ¹H NMR (400 MHz, DMSO-d₆): δ 9.13-9.09 (m, 1H), 7.89-7.86 (m, 1H), 7.73-7.68 (m, 1H), 4.80-479 (d, 2H), 4.34-4.28 (m, 2H), 2.13-2.10 (m, 3H).

### 3-Acetyl-8-fluoro-1-(methylamino)-1,3,4,5-tetrahydrobenzo[c][1,7]naphthyridin-6(2H)-one (Vf)

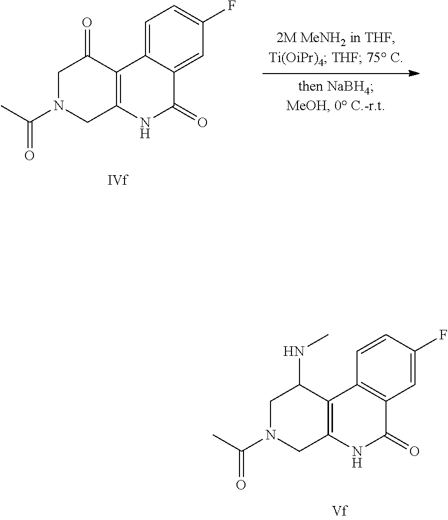 [SMILES: CC(=O)N1CC(=O)c2c([nH]c(=O)c3cc(F)ccc23)C1 ; CNC1CN(C(C)=O)Cc2[nH]c(=O)c3cc(F)ccc3c21]

Racemic 3-acetyl-8-fluoro-1-(methylamino)-1,3,4,5-tetrahydrobenzo[c][1,7]naphthyridin-6(2H)-one was synthesized in an analogous manner as described above for Vd, from 3-acetyl-8-fluoro-3,4-dihydrobenzo[c][1,7]naphthyridine-1,6(2H,5H)-dione (IVf) and methylamine. LCMS: m/z found 288.4 [M−H]⁺.

### N-(3-Acetyl-8-fluoro-6-oxo-1,2,3,4,5,6-hexahydrobenzo[c][1,7]naphthyridin-1-yl)-N-methyl-1H-indole-2-carboxamide (Compounds 68 and 69)

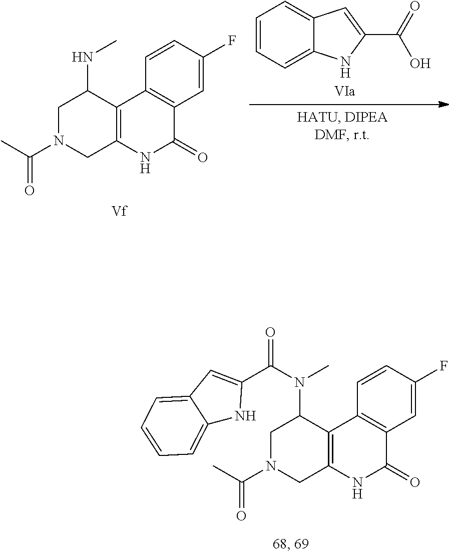

Racemic N-(3-acetyl-8-fluoro-6-oxo-1,2,3,4,5,6-hexahydrobenzo[c][1,7]naphthyridin-1-yl)-N-methyl-1H-indole-2-carboxamide was synthesized in an analogous manner as described above from racemic 3-acetyl-8-fluoro-1-(methylamino)-1,3,4,5-tetrahydrobenzo[c][1,7]naphthyridin-6(2H)-one (Vf) and 1H-indole-2-carboxylic acid (VIa). The enantiomers were subsequently separated by preparative SFC: Method isocratic, Mobile phase methanol:CO₂—45:65. Column: Chiralpak IC (30×250 mm), 5 μm, flow rate: 100 g/min.

Enantiomer I (Compound 68): LCMS: m/z found 433.2 [M+H]⁺, RT=3.95 min, (Method A); ¹H NMR (400 MHz, DMSO-d₆) δ 11.70 (br s, 2H), 7.92-7.89 (m, 1H), 7.65-7.59 (m, 2H), 7.46-7.48 (m, 2H), 7.20-7.23 (m, 1H), 7.04-7.06 (m, 1H), 6.86-6.95 (m, 1H), 5.87-5.90 (m, 1H), 5.02-4.18 (m, 4H), 2.91 (s, 3H), 2.08 (s, 3H); Chiral analytical SFC: RT=5.88 min, Column: Chiralpak IC-3 (4.6×150 mm) 3 μm, 30% of MeOH, Flow rate: 3.0 g/min.

Enantiomer II (Compound 69): LCMS: m/z found 433.2 [M+H]⁺, RT=3.95 min, (Method A); ¹H NMR (400 MHz, DMSO-d₆) δ 11.70 (br s, 2H), 7.92-7.89 (m, 1H), 7.65-7.59 (m, 2H), 7.46-7.48 (m, 2H), 7.20-7.23 (m, 1H), 7.04-7.06 (m, 1H), 6.86-6.95 (m, 1H), 5.87-5.90 (m, 1H), 5.02-4.18 (m, 4H), 2.91 (s, 3H), 2.08 (s, 3H); Chiral analytical SFC: RT=9.81 min, Column: Chiralpak IC-3 (4.6×150 mm) 3 μm, 30% of MeOH, Flow rate: 3.0 g/min.

### tert-Butyl 8,9-difluoro-1,6-dioxo-1,4,5,6-tetrahydrobenzo[c][1,7]naphthyridine-3(2H)-carboxylate (IVg)

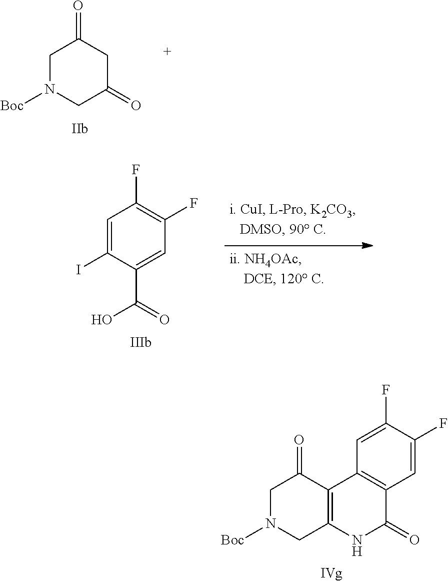 [SMILES: CC(C)(C)OC(=O)N1CC(=O)CC(=O)C1 ; O=C(O)c1cc(F)c(F)cc1I ; CC(C)(C)OC(=O)N1CC(=O)c2c([nH]c(=O)c3cc(F)c(F)cc23)C1]

tert-Butyl 8,9-difluoro-1,6-dioxo-1,4,5,6-tetrahydrobenzo[c][1,7]naphthyridine-3(2H)-carboxylate was synthesized in an analogous manner as described above for IVc, from tert-butyl 3,5-dioxopiperidine-1-carboxylate (Ib) and 4,5-difluoro-2-iodo-benzoic acid (Ib). ¹H NMR (400 MHz, DMSO-d₆): δ 12.45 (br s, 1H), 9.03-8.97 (m, 1H), 8.12 (dd, 1H), 4.71 (br s, 2H), 4.18 (br s, 2H), 1.42 (s, 9H).

### tert-Butyl 8,9-difluoro-1-(methylamino)-6-oxo-1,4,5,6-tetrahydrobenzo[c][1,7]naphthyridine-3(2H)-carboxylate (Vg)

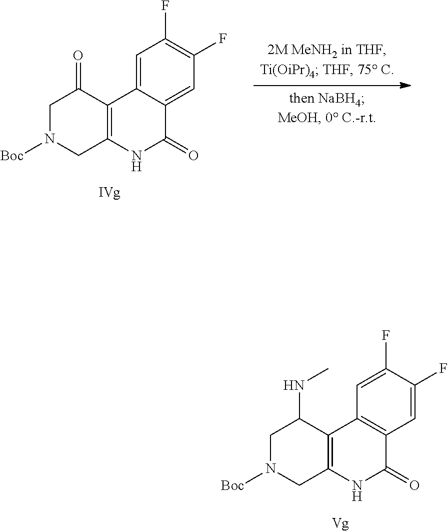 [SMILES: CC(C)(C)OC(=O)N1CC(=O)c2c([nH]c(=O)c3cc(F)c(F)cc23)C1 ; CNC1CN(C(=O)OC(C)(C)C)Cc2[nH]c(=O)c3cc(F)c(F)cc3c21]

Racemic tert-butyl 8,9-difluoro-1-(methylamino)-6-oxo-1,4,5,6-tetrahydrobenzo[c][1,7]naphthyridine-3(2H)-carboxylate was synthesized in an analogous manner as described above for Vc, from tert-butyl 8,9-difluoro-1,6-dioxo-1,4,5,6-tetrahydrobenzo[c][1,7]naphthyridine-3(2H)-carboxylate (IVg) and methylamine. LCMS: m/z found 366.3 [M+H]⁺.

### 3-(2-((tert-Butyldimethylsilyl)oxy)ethyl)-8,9-difluoro-1-(methylamino)-1,3,4,5-tetrahydrobenzo[c][1,7]naphthyridin-6(2H)-one (Vh)

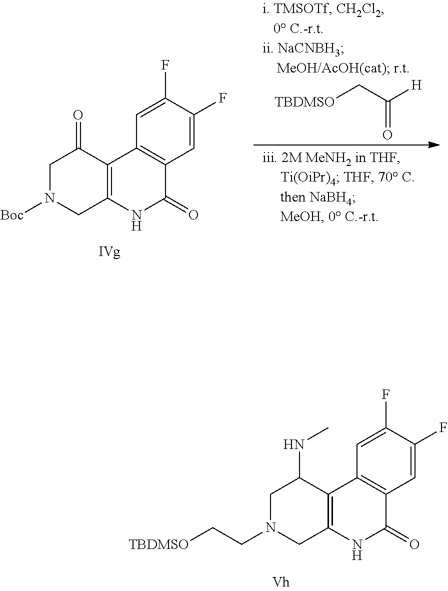 [SMILES: CC(C)(C)OC(=O)N1CC(=O)c2c([nH]c(=O)c3cc(F)c(F)cc23)C1 ; CC(C)(C)[Si](C)(C)OCC=O ; CNC1CN(CCO[Si](C)(C)C(C)(C)C)Cc2[nH]c(=O)c3cc(F)c(F)cc3c21]

Racemic 3-(2-((Tert-butyldimethylsilyl)oxy)ethyl)-8,9-difluoro-1-(methylamino)-1,3,4,5-tetrahydrobenzo[c][1,7]naphthyridin-6(2H)-one was prepared from tert-butyl 8,9-difluoro-1,6-dioxo-1,4,5,6-tetrahydrobenzo[c][1,7]naphthyridine-3(2H)-carboxylate (IVg), 2-((tert-butyldimethylsilyl)oxy)acetaldehyde, and methylamine by a procedure similar to the one described above for Vd. LCMS: m/z found 422.5 [M−H]⁻.

### 8,9-Difluoro-3-methyl-1-(methylamino)-1,3,4,5-tetrahydrobenzo[c][1,7]naphthyridin-6(2H)-one (Vi)

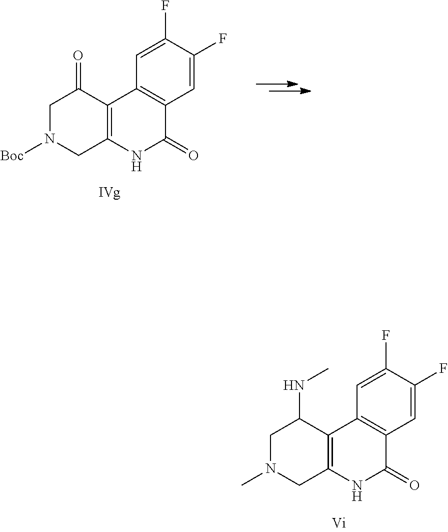 [SMILES: CC(C)(C)OC(=O)N1CC(=O)c2c([nH]c(=O)c3cc(F)c(F)cc23)C1 ; CNC1CN(C)Cc2[nH]c(=O)c3cc(F)c(F)cc3c21]

Racemic 8,9-difluoro-3-methyl-1-(methylamino)-1,3,4,5-tetrahydrobenzo[c][1,7]naphthyridin-6(2H)-one was synthesized in an analogous manner as described above for Ve, from tert-butyl 8,9-difluoro-1,6-dioxo-1,4,5,6-tetrahydrobenzo[c][1,7]naphthyridine-3(2H)-carboxylate (IVg), formaldehyde, and methylamine.

### 3-Acetyl-8,9-difluoro-1-(methylamino)-1,3,4,5-tetrahydrobenzo[c][1,7]naphthyridin-6(2H)-one (Vj)

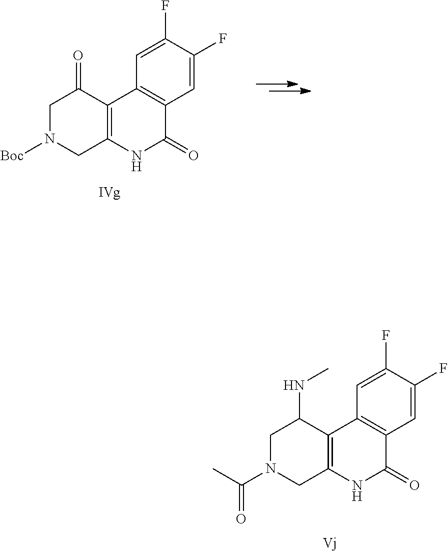 [SMILES: CC(C)(C)OC(=O)N1CC(=O)c2c([nH]c(=O)c3cc(F)c(F)cc23)C1 ; CNC1CN(C(C)=O)Cc2[nH]c(=O)c3cc(F)c(F)cc3c21]

Racemic 3-acetyl-8,9-difluoro-1-(methylamino)-1,3,4,5-tetrahydrobenzo[c][1,7]naphthyridin-6(2H)-one was synthesized in an analogous manner as described above for Vf, from tert-butyl 8,9-difluoro-1,6-dioxo-1,4,5,6-tetrahydrobenzo[c][1,7]naphthyridine-3(2H)-carboxylate (IVg), acetic anhydride, and methylamine. LCMS: m/z found 308.29 [M−H]⁺.

### N-(8,9-Difluoro-6-oxo-1,2,3,4,5,6-hexahydrobenzo[c][1,7]naphthyridin-1-yl)-N-methyl-1H-indole-2-carboxamide (Compounds 54 and 55)

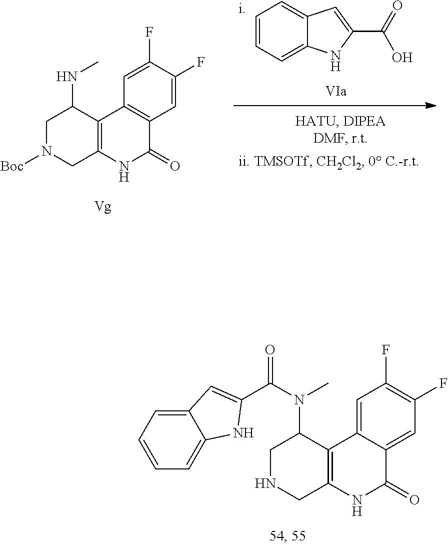 [SMILES: CNC1CN(C(=O)OC(C)(C)C)Cc2[nH]c(=O)c3cc(F)c(F)cc3c21 ; O=C(O)c1cc2ccccc2[nH]1 ; CN(C(=O)c1cc2ccccc2[nH]1)C1CNCc2[nH]c(=O)c3cc(F)c(F)cc3c21]

Racemic N-(8,9-difluoro-6-oxo-1,2,3,4,5,6-hexahydrobenzo[c][1,7]naphthyridin-1-yl)-N-methyl-1H-indole-2-carboxamide was synthesized in an analogous manner as described above, from racemic tert-butyl 8,9-difluoro-1-(methylamino)-6-oxo-1,4,5,6-tetrahydrobenzo[c][1,7]naphthyridine-3(2H)-carboxylate (Vg) and 1H-indole-2-carboxylic acid (VIa). The enantiomers were subsequently separated by preparative SFC: Method isocratic, Mobile phase (0.2% 7M Methanolic ammonia in Methanol):CO₂—40:60. Column: Chiralcel-ODH (30×250 mm), 5 μm, flow rate: 100 g/min.

Enantiomer I (Compound 54): LCMS: m/z found 409.2 [M+H]⁺, RT=3.17 min, (Method A); ¹H NMR (400 MHz, DMSO-d₆) δ 11.70 (br s, 2H), 8.10 (m, 1H), 7.66 (d, 1H), 7.47 (d, 1H), 7.29 (m, 1H), 7.21 (t, 1H), 6.84 (s, 1H), 5.96 (br s, 1H), 3.80 (d, 1H), 3.66 (d, 1H), 3.40 (m, 2H), 3.16 (s, 3H), 2.98 (m, 2H); Chiral analytical SFC: RT=2.57 min, Column: Chiralcel OD-3 (4.6×150 mm) 3 μm, 40% of (0.5% DEA in Methanol), Flow rate: 3.0 g/min.

Enantiomer II (Compound 55): LCMS: m/z found 409.2 [M+H]⁺, RT=3.17 min, (Method A); ¹H NMR (400 MHz, DMSO-d₆ δ 11.70 (br s, 2H), 8.10 (m, 1H), 7.66 (d, 1H), 7.47 (d, 1H), 7.29 (m, 1H), 7.21 (t, 1H), 6.84 (s, 1H), 5.96 (br s, 1H), 3.80 (d, 1H), 3.66 (d, 1H), 3.40 (m, 2H), 3.16 (s, 3H), 2.98 (m, 2H); Chiral analytical SFC: RT=3.77 min, Column: Chiralcel OD-3 (4.6×150 mm) 3 μm, 40% of (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-5-fluoro-N-methyl-1H-indole-2-carboxamide (Compounds 101 and 102)

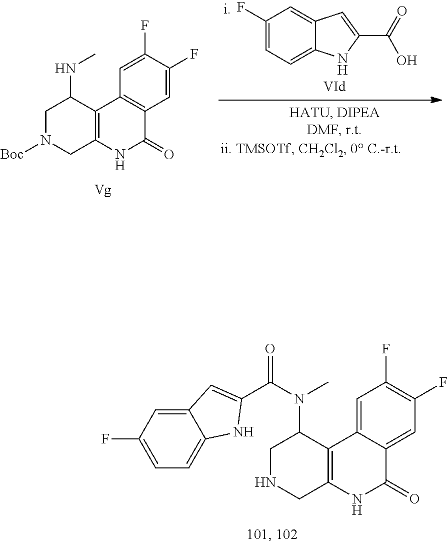 [SMILES: CNC1CN(C(=O)OC(C)(C)C)Cc2[nH]c(=O)c3cc(F)c(F)cc3c21 ; O=C(O)c1cc2cc(F)ccc2[nH]1 ; CN(C(=O)c1cc2cc(F)ccc2[nH]1)C1CNCc2[nH]c(=O)c3cc(F)c(F)cc3c21]

Racemic N-(8,9-difluoro-6-oxo-1,2,3,4,5,6-hexahydrobenzo[c][1,7]naphthyridin-1-yl)-5-fluoro-N-methyl-1H-indole-2-carboxamide was synthesized in an analogous manner as described above, from racemic tert-butyl 8,9-difluoro-1-(methylamino)-6-oxo-1,4,5,6-tetrahydrobenzo[c][1,7]naphthyridine-3(2H)-carboxylate (Vg) and 5-fluoro-1H-indole-2-carboxylic acid (VId). The enantiomers were subsequently separated by preparative SFC: Method isocratic, Mobile phase (Methanol:Acetonitrile (1:1)):CO₂—40:60. Column: Chiralcel-OX-H (30×250 mm), 5 μm, flow rate: 110 g/min.

Enantiomer I (Compound 101): LCMS: m/z found 427.2 [M+H]⁺, RT=4.46 min, (Method A); ¹H NMR (400 MHz, DMSO-d₆) δ 11.82 (br s, 2H), 8.12-8.06 (m, 1H), 7.48-7.44 (m, 1H), 7.36-7.24 (m, 2H), 7.09-7.05 (m, 1H), 6.88 (s, 1H), 5.67 (s, 1H), 3.78 (d, 1H), 3.63 (d, 1H), 3.17 (s, 3H), 3.17-3.27 (m, 3H); Chiral analytical SFC: RT=3.14 min, Column: Chiralcel OX-3 (4.6×150 mm) 3 μm, 40% of (0.2% 7 M Methanolic ammonia in ACN:MeOH (1:1)), Flow rate: 3.0 g/min.

Enantiomer II (Compound 102): LCMS: m/z found 427.2 [M+H]⁺, RT=4.46 min, (Method A); ¹H NMR (400 MHz, DMSO-d₆) δ 11.82 (br s, 2H), 8.12-8.06 (m, 1H), 7.48-7.44 (m, 1H), 7.36-7.24 (m, 2H), 7.09-7.05 (m, 1H), 6.88 (s, 1H), 5.67 (s, 1H), 3.78 (d, 1H), 3.63 (d, 1H), 3.17 (s, 3H), 3.17-3.27 (m, 3H); Chiral analytical SFC: RT=5.01 min, Column: Chiralcel OX-3 (4.6×150 mm) 3 μm, 40% of (0.2% 7 M Methanolic ammonia in ACN:MeOH (1:1)), Flow rate: 3.0 g/min.

### N-(8,9-Difluoro-6-oxo-1,2,3,4,5,6-hexahydrobenzo[c][1,7]naphthyridin-1-yl)-4-fluoro-N-methyl-1H-indole-2-carboxamide (Compounds 105 and 106)

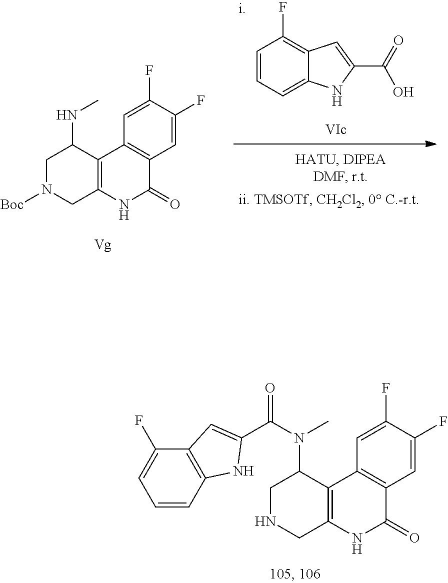 [SMILES: CNC1CN(C(=O)OC(C)(C)C)Cc2[nH]c(=O)c3cc(F)c(F)cc3c21 ; O=C(O)c1cc2c(F)cccc2[nH]1 ; CN(C(=O)c1cc2c(F)cccc2[nH]1)C1CNCc2[nH]c(=O)c3cc(F)c(F)cc3c21]

Racemic N-(8,9-difluoro-6-oxo-1,2,3,4,5,6-hexahydrobenzo[c][1,7]naphthyridin-1-yl)-4-fluoro-N-methyl-1H-indole-2-carboxamide was synthesized in an analogous manner as described above, from racemic tert-butyl 8,9-difluoro-1-(methylamino)-6-oxo-1,4,5,6-tetrahydrobenzo[c][1,7]naphthyridine-3(2H)-carboxylate (Vg) and 4-fluoro-1H-indole-2-carboxylic acid (VIc). The enantiomers were subsequently separated by preparative SFC: Method isocratic, Mobile phase (0.2% 7 M Methanolic ammonia in ACN:MeOH (1:1)):CO₂—40:60. Column: Chiralcel-OX-H (30×250 mm), 5 μm, flow rate: 70 g/min.

Enantiomer I (Compound 105): LCMS: m/z found 427.2 [M+H]⁺, RT=4.73 min, (Method A); ¹H NMR (400 MHz, DMSO-d₆) δ 12.04 (brs, 1H), 11.64 (brs, 1H); 8.13-8.06 (m, 1H), 7.31-7.25 (m, 2H), 7.21-7.16 (m, 1H), 6.94 (s, 1H), 6.84-6.80 (m, 1H), 5.67 (s, 1H), 3.79 (d, 1H), 3.63 (d, 1H), 3.16-2.77 (m, 6H); Chiral analytical SFC: RT=3.26 min, Column: Chiralcel OX-3 (4.6×150 mm) 3 μm, 40% of (0.2% 7 M Methanolic ammonia in ACN:MeOH (1:1)), Flow rate: 3.0 g/min.

Enantiomer II (Compound 106): LCMS: m/z found 427.2 [M+H]⁺, RT=4.73 min, (Method A); ¹H NMR (400 MHz, DMSO-d₆) δ 12.04 (brs, 1H), 11.64 (brs, 1H); 8.13-8.06 (m, 1H), 7.31-7.25 (m, 2H), 7.21-7.16 (m, 1H), 6.94 (s, 1H), 6.84-6.80 (m, 1H), 5.67 (s, 1H), 3.79 (d, 1H), 3.63 (d, 1H), 3.16-2.77 (m, 6H); Chiral analytical SFC: RT=5.66 min, Column: Chiralcel OX-3 (4.6×150 mm) 3 μm, 40% of (0.2% 7 M Methanolic ammonia in ACN:MeOH (1:1)), Flow rate: 3.0 g/min.

### N-(8,9-Difluoro-6-oxo-1,2,3,4,5,6-hexahydrobenzo[c][1,7]naphthyridin-1-yl)-6-fluoro-N-methyl-1H-indole-2-carboxamide (Compounds 107 and 108)

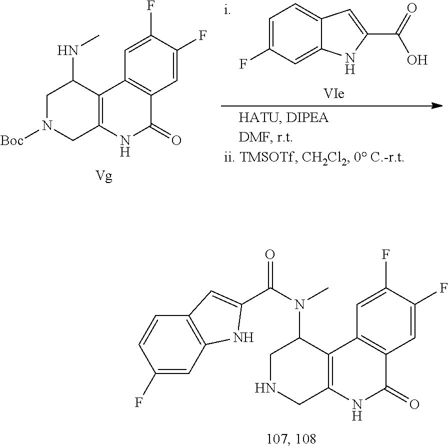 [SMILES: CNC1CN(C(=O)OC(C)(C)C)Cc2[nH]c(=O)c3cc(F)c(F)cc3c21 ; O=C(O)c1cc2ccc(F)cc2[nH]1 ; CN(C(=O)c1cc2ccc(F)cc2[nH]1)C1CNCc2[nH]c(=O)c3cc(F)c(F)cc3c21]

Racemic N-(8,9-difluoro-6-oxo-1,2,3,4,5,6-hexahydrobenzo[c][1,7]naphthyridin-1-yl)-6-fluoro-N-methyl-1H-indole-2-carboxamide was synthesized in an analogous manner as described above, from racemic tert-butyl 8,9-difluoro-1-(methylamino)-6-oxo-1,4,5,6-tetrahydrobenzo[c][1,7]naphthyridine-3(2H)-carboxylate (Vg) and 6-fluoro-1H-indole-2-carboxylic acid (VIe). The enantiomers were subsequently separated by preparative SFC: Method isocratic, Mobile phase (0.2% 7 M Methanolic ammonia in ACN:MeOH (1:1)):CO₂—40:60. Column: Chiralcel-OX-H (30×250 mm), 5 μm, flow rate: 70 g/min.

Enantiomer I (Compound 107): LCMS: m/z found 427.2 [M+H]⁺, RT=4.74 min, (Method A); ¹H NMR (400 MHz, DMSO-d₆) δ 11.8 (brs, 2H), 8.12-8.08 (m, 1H), 7.64-7.61 (m, 1H), 7.30-7.25 (m, 1H), 7.16 (d, 1H), 6.94-6.90 (m, 2H), 5.68 (s, 1H), 3.78 (d, 1H), 3.63 (d, 1H), 3.15-2.75 (m, 6H); Chiral analytical SFC: RT=3.72 min, Column: Chiralcel OX-3 (4.6×150 mm) 3 μm, 40% of (0.2% 7 M Methanolic ammonia in ACN:MeOH (1:1)), Flow rate: 3.0 g/min.

Enantiomer II (Compound 108): LCMS: m/z found 427.2 [M+H]⁺, RT=4.74 min, (Method A); ¹H NMR (400 MHz, DMSO-d₆) δ 11.8 (brs, 2H), 8.12-8.08 (m, 1H), 7.64-7.61 (m, 1H), 7.30-7.25 (m, 1H), 7.16 (d, 1H), 6.94-6.90 (m, 2H), 5.68 (s, 1H), 3.78 (d, 1H), 3.63 (d, 1H), 3.15-2.75 (m, 6H); Chiral analytical SFC: RT=5.55 min, Column: Chiralcel OX-3 (4.6×150 mm) 3 μm, 40% of (0.2% 7 M Methanolic ammonia in ACN:MeOH (1:1)), Flow rate: 3.0 g/min.

### N-(8,9-Difluoro-6-oxo-1,2,3,4,5,6-hexahydrobenzo[c][1,7]naphthyridin-1-yl)-7-fluoro-N-methyl-1H-indole-2-carboxamide (Compounds 113 and 114)

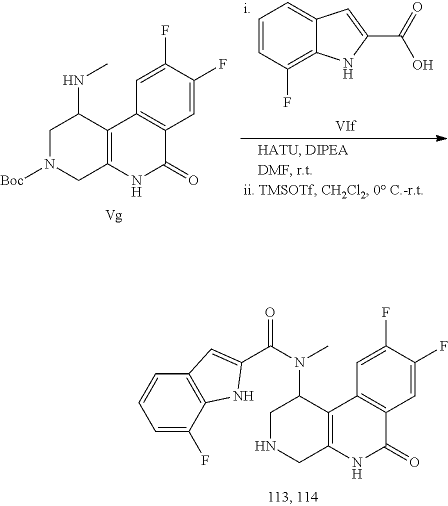

Racemic N-(8,9-difluoro-6-oxo-1,2,3,4,5,6-hexahydrobenzo[c][1,7]naphthyridin-1-yl)-7-fluoro-N-methyl-1H-indole-2-carboxamide was synthesized in an analogous manner as described above, from racemic tert-butyl 8,9-difluoro-1-(methylamino)-6-oxo-1,4,5,6-tetrahydrobenzo[c][1,7]naphthyridine-3(2H)-carboxylate (Vg) and 7-fluoro-1H-indole-2-carboxylic acid (VIf). The enantiomers were subsequently separated by preparative SFC: Method isocratic, Mobile phase (0.2% 7 M Methanolic ammonia in ACN:MeOH (1:1)):CO₂—30:70. Column: Chiralcel-OJ-3 (30×250 mm), 5 μm, flow rate: 100 g/min.

Enantiomer I (Compound 113): LCMS: m/z found 427.2 [M+H]⁺, RT=4.81 min, (Method A); ¹H NMR (400 MHz, DMSO-d₆) δ 12.1 (brs, 1H), 11.62 (br s, 1H), 8.13-8.09 (m, 1H), 7.42 (d, 1H), 7.31-7.26 (m, 1H), 7.05-7.0 (m, 2H), 6.92 (d, 1H), 5.64 (s, 1H), 3.78 (d, 1H), 3.63 (d, 1H), 3.31-2.77 (m, 6H); Chiral analytical SFC: RT=1.46 min, Column: Chiralcel OJ-3 (4.6×150 mm) 3 μm, 30% of (0.2% 7 M Methanolic ammonia in ACN:MeOH (1:1)), Flow rate: 3.0 g/min.

Enantiomer II (Compound 114): LCMS: m/z found 427.2 [M+H]⁺, RT=4.81 min, (Method A); ¹H NMR (400 MHz, DMSO-d₆) δ 12.1 (brs, 1H), 11.62 (br s, 1H), 8.13-8.09 (m, 1H), 7.42 (d, 1H), 7.31-7.26 (m, 1H), 7.05-7.0 (m, 2H), 6.92 (d, 1H), 5.64 (s, 1H), 3.78 (d, 1H), 3.63 (d, 1H), 3.31-2.77 (m, 6H); Chiral analytical SFC: RT=2.11 min, Column: Chiralcel OJ-3 (4.6×150 mm) 3 μm, 30% of (0.2% 7 M Methanolic ammonia in ACN:MeOH (1:1)), Flow rate: 3.0 g/min.

### N-(8,9-Difluoro-6-oxo-1,2,3,4,5,6-hexahydrobenzo[c][1,7]naphthyridin-1-yl)-5,6-difluoro-N-methyl-1H-indole-2-carboxamide (Compounds 115 and 116)

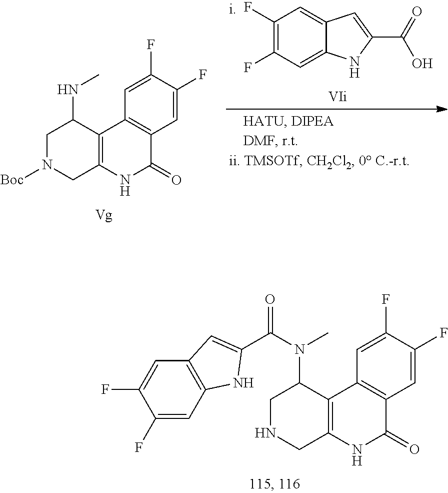 [SMILES: CNC1CN(C(=O)OC(C)(C)C)Cc2[nH]c(=O)c3cc(F)c(F)cc3c21 ; O=C(O)c1cc2cc(F)c(F)cc2[nH]1 ; CN(C(=O)c1cc2cc(F)c(F)cc2[nH]1)C1CNCc2[nH]c(=O)c3cc(F)c(F)cc3c21]

Racemic N-(8,9-difluoro-6-oxo-1,2,3,4,5,6-hexahydrobenzo[c][1,7]naphthyridin-1-yl)-5,6-difluoro-N-methyl-1H-indole-2-carboxamide was synthesized in an analogous manner as described above, from racemic tert-butyl 8,9-difluoro-1-(methylamino)-6-oxo-1,4,5,6-tetrahydrobenzo[c][1,7]naphthyridine-3(2H)-carboxylate (Vg) and 5,6-difluoro-1H-indole-2-carboxylic acid (VIi). The enantiomers were subsequently separated by preparative SFC: Method isocratic, Mobile phase (0.2% 7 M Methanolic ammonia in ACN:MeOH (1:1)):CO₂—40:60. Column Chiralcel-OX-H (30×250 mm), 5 μm, flow rate: 100 g/min.

Enantiomer I (Compound 115): LCMS: m/z found 445.2 [M+H]⁺, RT=4.89 min, (Method A); ¹H NMR (400 MHz, DMSO-d₆) δ 11.82 (br s, 2H), 8.12-8.08 (m, 1H), 7.63-7.58 (m, 1H), 7.40-7.35 (m, 1H), 7.29-7.25 (m, 1H), 6.92 (s, 1H), 5.66 (s, 1H), 3.82 (d, 1H), 3.63 (d, 1H), 3.16-2.66 (m, 6H); Chiral analytical SFC: RT=3.21 min, Column: Chiralcel OX-3 (4.6×150 mm) 3 μm, 40% of (0.2% 7 M Methanolic ammonia in ACN:MeOH (1:1)), Flow rate: 3.0 g/min.

Enantiomer II (Compound 116): LCMS: m/z found 445.2 [M+H]⁺, RT=4.89 min, (Method A); ¹H NMR (400 MHz, DMSO-d₆) δ 11.82 (br s, 2H), 8.12-8.08 (m, 1H), 7.63-7.58 (m, 1H), 7.40-7.35 (m, 1H), 7.29-7.25 (m, 1H), 6.92 (s, 1H), 5.66 (s, 1H), 3.82 (d, 1H), 3.63 (d, 1H), 3.16-2.66 (m, 6H); Chiral analytical SFC: RT=5.14 min, Column: Chiralcel OX-3 (4.6×150 mm) 3 μm, 40% of (0.2% 7 M Methanolic ammonia in ACN:MeOH (1:1)), Flow rate: 3.0 g/min.

### N-(8,9-Difluoro-6-oxo-1,2,3,4,5,6-hexahydrobenzo[c][1,7]naphthyridin-1-yl)-4,6-difluoro-N-methyl-1H-indole-2-carboxamide (Compounds 117 and 118)

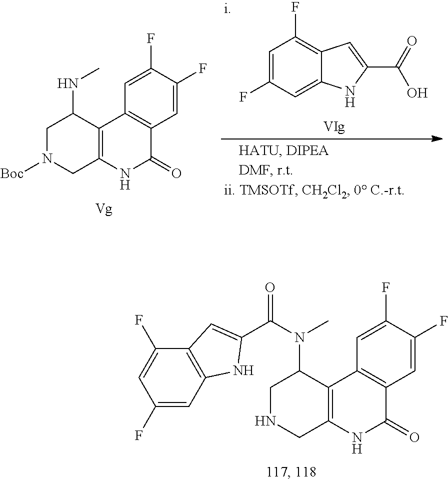 [SMILES: CNC1CN(C(=O)OC(C)(C)C)Cc2[nH]c(=O)c3cc(F)c(F)cc3c21 ; O=C(O)c1cc2c(F)cc(F)cc2[nH]1 ; CN(C(=O)c1cc2c(F)cc(F)cc2[nH]1)C1CNCc2[nH]c(=O)c3cc(F)c(F)cc3c21]

Racemic N-(8,9-difluoro-6-oxo-1,2,3,4,5,6-hexahydrobenzo[c][1,7]naphthyridin-1-yl)-4,6-difluoro-N-methyl-1H-indole-2-carboxamide was synthesized in an analogous manner as described above, from racemic tert-butyl 8,9-difluoro-1-(methylamino)-6-oxo-1,4,5,6-tetrahydrobenzo[c][1,7]naphthyridine-3(2H)-carboxylate (Vg) and 4,6-difluoro-1H-indole-2-carboxylic acid (VIg). The enantiomers were subsequently separated by preparative SFC: Method isocratic, Mobile phase (0.2% 7 M Methanolic ammonia in ACN:MeOH (1:1)):CO₂—40:60. Column Chiralcel-OX-H (30×250 mm), 5 μm, flow rate: 70 g/min.

Enantiomer I (Compound 117): LCMS: m/z found 445.2 [M+H]⁺, RT=4.93 min, (Method A); ¹H NMR (400 MHz, DMSO-d₆) δ 12.12 (brs, 2H); 8.13-8.08 (m, 1H), 7.29-7.24 (t, 1H), 7.61 (d, 1H), 6.97 (s, 1H), 6.92-6.87 (m, 1H), 5.68 (s, 1H), 3.78 (d, 1H), 3.63 (d, 1H), 3.15-2.76 (m, 6H); Chiral analytical SFC: RT=2.92 min, Column: Chiralcel OX-3 (4.6×150 mm) 3 μm, 30% of (0.2% 7M Methanolic ammonia in ACN:MeOH (1:1)), Flow rate: 3.0 g/min.

Enantiomer II (Compound 118): LCMS: m/z found 445.2 [M+H]⁺, RT=4.93 min, (Method A); ¹H NMR (400 MHz, DMSO-d₆) δ 12.12 (brs, 2H); 8.13-8.08 (m, 1H), 7.29-7.24 (t, 1H), 7.61 (d, 1H), 6.97 (s, 1H), 6.92-6.87 (m, 1H), 5.68 (s, 1H), 3.78 (d, 1H), 3.63 (d, 1H), 3.15-2.76 (m, 6H); Chiral analytical SFC: RT=4.74 min, Column: Chiralcel OX-3 (4.6×150 mm) 3 μm, 30% of (0.2% 7 M Methanolic ammonia in ACN:MeOH (1:1)), Flow rate: 3.0 g/min.

### N-(8,9-Difluoro-6-oxo-1,2,3,4,5,6-hexahydrobenzo[c][1,7]naphthyridin-1-yl)-4,5-difluoro-N-methyl-1H-indole-2-carboxamide (Compounds 119 and 120)

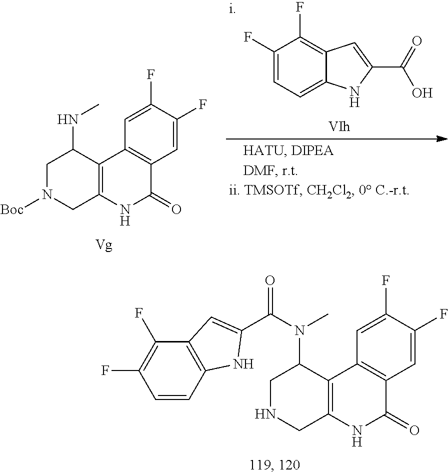 [SMILES: CNC1CN(C(=O)OC(C)(C)C)Cc2[nH]c(=O)c3cc(F)c(F)cc3c21 ; O=C(O)c1cc2c(F)c(F)ccc2[nH]1 ; CN(C(=O)c1cc2c(F)c(F)ccc2[nH]1)C1CNCc2[nH]c(=O)c3cc(F)c(F)cc3c21]

Racemic N-(8,9-difluoro-6-oxo-1,2,3,4,5,6-hexahydrobenzo[c][1,7]naphthyridin-1-yl)-4,5-difluoro-N-methyl-1H-indole-2-carboxamide was synthesized in an analogous manner as described above, from racemic tert-butyl 8,9-difluoro-1-(methylamino)-6-oxo-1,4,5,6-tetrahydrobenzo[c][1,7]naphthyridine-3(2H)-carboxylate (Vg) and 4,5-difluoro-1H-indole-2-carboxylic acid (VIh). The enantiomers were subsequently separated by preparative SFC: Method isocratic, Mobile phase (0.2% 7 M Methanolic ammonia in ACN:MeOH (1:1)):CO₂—40:60. Column Chiralcel-OX-H (30×250 mm) 5 μm, flow rate: 70 g/min.

Enantiomer I (Compound 119): LCMS: m/z found 445.2 [M+H]⁺, RT=4.89 min, (Method A); ¹H NMR (400 MHz, DMSO-d₆) δ 12.1 (brs, 1H); 11.6 (brs, 1H), 8.13-8.05 (m, 1H), 7.29-7.21 (m, 3H), 7.01 (s, 1H), 5.66 (s, 1H), 3.78 (d, 1H), 3.63 (d, 1H), 3.15-2.76 (m, 6H); Chiral analytical SFC: RT=2.83 min, Column: Chiralcel OX-3 (4.6×150 mm) 3 μm, 30% of (0.2% 7 M Methanolic ammonia in ACN:MeOH (1:1)), Flow rate: 3.0 g/min.

Enantiomer II (Compound 120): LCMS: m/z found 445.2 [M+H]⁺, RT=4.89 min, (Method A); ¹H NMR (400 MHz, DMSO-d₆) δ 12.1 (brs, 1H); 11.6 (brs, 1H), 8.13-8.05 (m, 1H), 7.29-7.21 (m, 3H), 7.01 (s, 1H), 5.66 (s, 1H), 3.78 (d, 1H), 3.63 (d, 1H), 3.15-2.76 (m, 6H); Chiral analytical SFC: RT=4.68 min, Column: Chiralcel OX-3 (4.6×150 mm) 3 μm, 30% of (0.2% 7 M Methanolic ammonia in ACN:MeOH (1:1)), Flow rate: 3.0 g/min.

### 6-Chloro-N-(8,9-difluoro-6-oxo-1,2,3,4,5,6-hexahydrobenzo[c][1,7]naphthyridin-1-yl)-N-methylindolizine-2-carboxamide (Compounds 121 and 122)

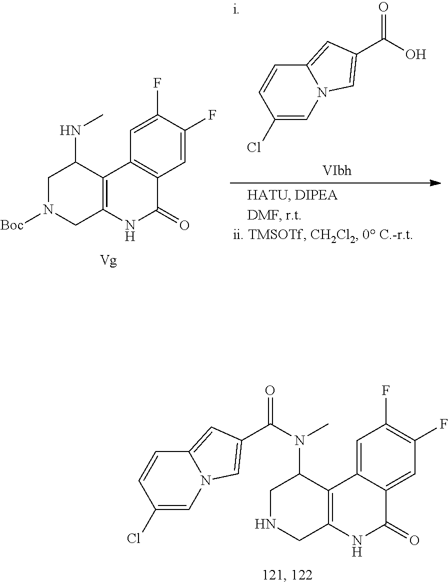 [SMILES: CNC1CN(C(=O)OC(C)(C)C)Cc2[nH]c(=O)c3cc(F)c(F)cc3c21 ; O=C(O)c1cc2ccc(Cl)cn2c1 ; CN(C(=O)c1cc2ccc(Cl)cn2c1)C1CNCc2[nH]c(=O)c3cc(F)c(F)cc3c21]

Racemic 6-chloro-N-(8,9-difluoro-6-oxo-1,2,3,4,5,6-hexahydrobenzo[c][1,7]naphthyridin-1-yl)-N-methylindolizine-2-carboxamide was synthesized in an analogous manner as described above, from racemic tert-butyl 8,9-difluoro-1-(methylamino)-6-oxo-1,4,5,6-tetrahydrobenzo[c][1,7]naphthyridine-3(2H)-carboxylate (Vg) and 6-chloroindolizine-2-carboxylic acid (VIbh). The enantiomers were subsequently separated by preparative SFC: Method isocratic, Mobile phase (0.2% 7 M Methanolic ammonia in ACN:MeOH (1:1)):CO₂—50:50. Column Chiralcel-OX-H (30×250 mm), 5μ, flow rate: 120 g/min.

Enantiomer I (Compound 121): LCMS: m/z found 443.2/445.2 [M+H]⁺, RT=4.78 min, (Method A); ¹H NMR (400 MHz, DMSO-d₆) δ 11.5 (brs, 1H), 8.52 (s, 1H), 8.13-8.03 (m, 1H), 7.90 (s, 1H), 7.48 (d, 1H), 7.31-7.26 (m, 1H), 6.78-6.72 (m, 2H), 5.63 (s, 1H), 3.75 (d, 1H), 3.61 (d, 1H), 3.14 (s, 2H), 2.99 (s, 3H), 2.75 (s, 1H); Chiral analytical SFC: RT=3.55 min, Column: Chiralcel OX-3 (4.6×150 mm) 3 μm, 50% of (0.2% 7M Methanolic ammonia in ACN:MeOH (1:1)), Flow rate: 3.0 g/min.

Enantiomer II (Compound 122): LCMS: m/z found 443.2/445.2 [M+H]⁺, RT=4.78 min, (Method A); ¹H NMR (400 MHz, DMSO-d₆) δ 11.5 (brs, 1H), 8.52 (s, 1H), 8.13-8.03 (m, 1H), 7.90 (s, 1H), 7.48 (d, 1H), 7.31-7.26 (m, 1H), 6.78-6.72 (m, 2H), 5.63 (s, 1H), 3.75 (d, 1H), 3.61 (d, 1H), 3.14 (s, 2H), 2.99 (s, 3H), 2.75 (s, 1H); Chiral analytical SFC: RT 20=5.04 min, Column: Chiralcel OX-3 (4.6×150 mm) 3 μm, 50% of (0.2% 7 M Methanolic ammonia in ACN:MeOH (1:1)), Flow rate: 3.0 g/min.

### N-(8,9-Difluoro-6-oxo-1,2,3,4,5,6-hexahydrobenzo[c][1,7]naphthyridin-1-yl)-8-fluoro-N-methylindolizine-2-carboxamide (Compounds 160 and 161)

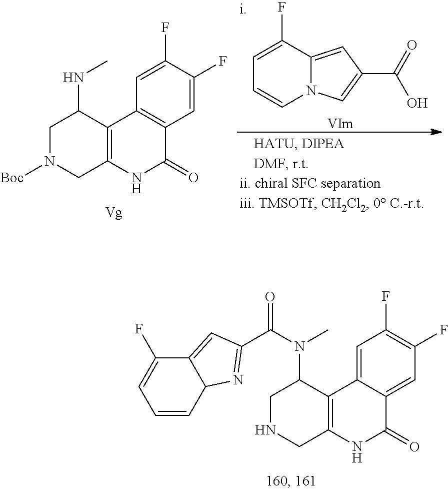

Step i: Racemic tert-butyl 8,9-difluoro-1-(8-fluoro-N-methylindolizine-2-carboxamido)-6-oxo-1,2,5,6-tetrahydrobenzo[c][1,7]naphthyridine-3(4H)-carboxylate was synthesized in an analogous manner as described above (Compounds 77, 78), from racemic tert-butyl 8,9-difluoro-1-(methylamino)-6-oxo-1,4,5,6-tetrahydrobenzo[c][1,7]naphthyridine-3(2H)-carboxylate (Vg) and 8-fluoroindolizine-2-carboxylic acid (VIm).

Step ii: The enantiomers of tert-butyl 8,9-difluoro-1-(8-fluoro-N-methylindolizine-2-carboxamido)-6-oxo-1,2,5,6-tetrahydrobenzo[c][1,7]naphthyridine-3(4H)-carboxylate were subsequently separated by chiral preparative SFC: method isocratic, mobile phase methanol:CO₂—30:70, Column: Chiralcel OX-H (30×250 mm), 5 μm, flow rate: 100 g/min.

Step iii: Each individual enantiomer of tert-butyl 8,9-difluoro-1-(8-fluoro-N-methylindolizine-2-carboxamido)-6-oxo-1,2,5,6-tetrahydrobenzo[c][1,7]naphthyridine-3(4H)-carboxylate isolated as described above was converted to the final product in an analogous manner as described above for Compounds 3 and 4 (Step ii) and purified by SFC: method isocratic, mobile phase methanol:CO₂—40:60, Column: DCPAK-P4VP (21×250 mm), 5 μm, flow rate: 65 g/min.

Enantiomer I (Compound 160): LCMS: m/z found 427.3 [M+H]⁺, RT=3.93 min, (Method A); ¹H NMR (400 MHz, DMSO-d₆) δ 11.61 (s, 1H), 8.15-8.06 (m, 3H), 7.32-7.27 (m, 1H), 6.75 (s, 1H), 6.65-6.61 (m, 2H), 5.62 (br s, 1H), 3.80-3.62 (m, 2H), 3.15-3.11 (m, 2H), 3.02 (s, 3H), 2.80-2.76 (m, 1H); Chiral analytical SFC: RT=3.32 min, Column: Chiralcel OJ-3 (4.6×150 mm) 3 μm, 15% (0.5% DEA in Methanol), Flow rate: 3.0 g/min.

Enantiomer II (Compound 161): LCMS: m/z found 427.3 [M+H]⁺, RT=3.93 min, (Method A); ¹H NMR (400 MHz, DMSO-d₆) δ 11.61 (s, 1H), 8.15-8.06 (m, 3H), 7.32-7.27 (m, 1H), 6.75 (s, 1H), 6.65-6.61 (m, 2H), 5.62 (br s, 1H), 3.80-3.62 (m, 2H), 3.15-3.11 (m, 2H), 3.02 (s, 3H), 2.80-2.76 (m, 1H); Chiral analytical SFC: RT=4.30 min, Column: Chiralcel OJ-3 (4.6×150 mm) 3 μm, 15% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-7-fluoro-N-methylindolizine-2-carboxamide (Compounds 162 and 163)

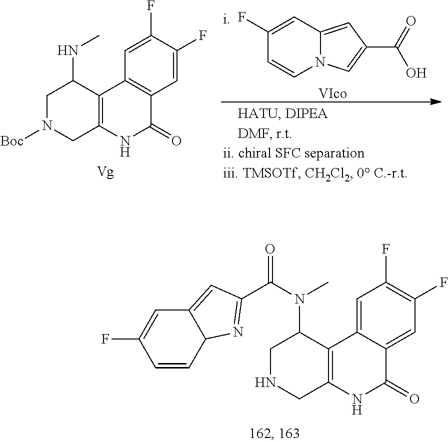

Individual enantiomers of N-(8,9-difluoro-6-oxo-1,2,3,4,5,6-hexahydrobenzo[c][1,7]naphthyridin-1-yl)-7-fluoro-N-methylindolizine-2-carboxamide were prepared in an analogous manner as described above (for Compounds 160, 161), from racemic tert-butyl 8,9-difluoro-1-(methylamino)-6-oxo-1,4,5,6-tetrahydrobenzo[c][1,7]naphthyridine-3(2H)-carboxylate (Vg) and 7-fluoroindolizine-2-carboxylic acid (VIco).

Enantiomer I (Compound 162): LCMS: m/z found 427.3 [M+H]⁺, RT=3.87 min, (Method A); ¹H NMR (400 MHz, DMSO-d₆) δ 11.61 (s, 1H), 8.31 (t, 1H), 8.10 (t, 1H), 7.87 (s, 1H), 7.31-7.21 (m, 2H), 6.67 (t, 1H), 6.56 (s, 1H) 5.62 (s, 1H), 3.77 (d, 1H), 3.63 (d, 1H), 3.28-3.20 (m, 2H), 2.98 (s, 3H), 2.74 (br s, 1H); Chiral analytical SFC: RT=5.80 min, Column: Chiralpak IC-3 (4.6×150 mm) 3 μm, 40% (0.5% DEA in Methanol), Flow rate: 3.0 g/min.

Enantiomer II (Compound 163): LCMS: m/z found 427.3 [M+H]⁺, RT=3.87 min, (Method A); ¹H NMR (400 MHz, DMSO-d₆) δ 11.61 (s, 1H), 8.31 (t, 1H), 8.10 (t, 1H), 7.87 (s, 1H), 7.31-7.21 (m, 2H), 6.67 (t, 1H), 6.56 (s, 1H) 5.62 (s, 1H), 3.77 (d, 1H), 3.63 (d, 1H), 3.28-3.20 (m, 2H), 2.98 (s, 3H), 2.74 (br s, 1H); Chiral analytical SFC: RT=6.97 min, Column: Chiralpak IC-3 (4.6×150 mm) 3 μm, 40% (0.5% DEA in Methanol), Flow rate: 3.0 g/min.

### 4-Bromo-N-(8,9-difluoro-6-oxo-1,2,3,4,5,6-hexahydrobenzo[c][1,7]naphthyridin-1-yl)-3,5-difluoro-N-methylbenzamide (Compounds 164 and 165)

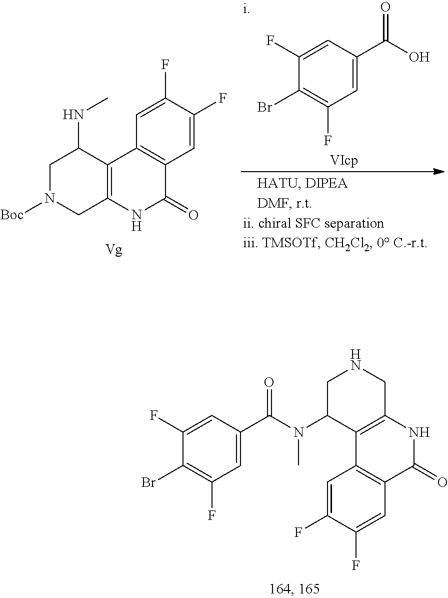

Individual enantiomers of 4-bromo-N-(8,9-difluoro-6-oxo-1,2,3,4,5,6-hexahydrobenzo[c][1,7]naphthyridin-1-yl)-3,5-difluoro-N-methylbenzamide were prepared in an analogous manner as described above (for Compounds 160, 161), from racemic tert-butyl 8,9-difluoro-1-(methylamino)-6-oxo-1,4,5,6-tetrahydrobenzo[c][1,7]naphthyridine-3(2H)-carboxylate (Vg) and 4-bromo-3,5-difluorobenzoic acid (VIcp).

Enantiomer I (Compound 164): LCMS: m/z found 484.2 [M+H]⁺, RT=4.67 min, (Method A); ¹H NMR (400 MHz, DMSO-d₆) δ 11.61 (s, 1H), 8.10 (t, 1H), 7.36-7.29 (m, 3H), 5.52-4.49 (br s, 1H), 3.76 (d, 1H), 3.62 (d, 1H), 3.31-3.26 (m, 1H), 3.12-3.08 (m, 1H), 2.78-2.72 (m, 1H), 2.71 (s, 3H); Chiral analytical SFC: RT=3.15 min, Column: Chiralcel OX-3 (4.6×150 mm) 3 μm, 30% (0.5% DEA in Methanol), Flow rate: 3.0 g/min.

Enantiomer II (Compound 165): LCMS: m/z found 484.2 [M+H]⁺, RT=4.67 min, (Method A); ¹H NMR (400 MHz, DMSO-d₆) δ 11.61 (s, 1H), 8.10 (t, 1H), 7.36-7.29 (m, 3H), 5.52-4.49 (br s, 1H), 3.76 (d, 1H), 3.62 (d, 1H), 3.31-3.26 (m, 1H), 3.12-3.08 (m, 1H), 2.78-2.72 (m, 1H), 2.71 (s, 3H); Chiral analytical SFC: RT=3.76 min, Column: Chiralcel OX-3 (4.6×150 mm) 3 μm, 30% (0.5% DEA in Methanol), Flow rate: 3.0 g/min.

### 4-Chloro-N-(8,9-difluoro-6-oxo-1,2,3,4,5,6-hexahydrobenzo[c][1,7]naphthyridin-1-yl)-3,5-difluoro-N-methylbenzamide (Compounds 168 and 169)

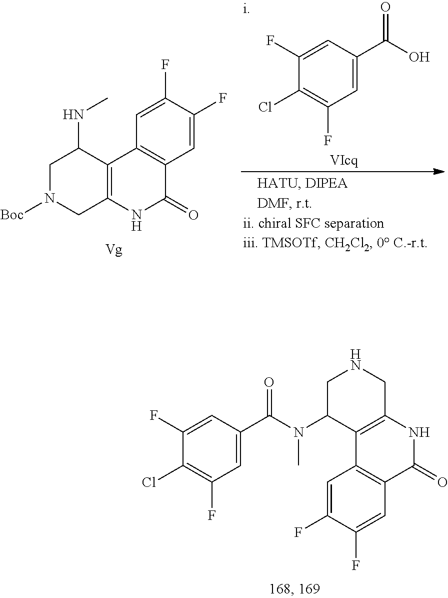

Individual enantiomers of 4-chloro-N-(8,9-difluoro-6-oxo-1,2,3,4,5,6-hexahydrobenzo[c][1,7]naphthyridin-1-yl)-3,5-difluoro-N-methylbenzamide were prepared in an analogous manner as described above (for Compounds 160, 161), from racemic tert-butyl 8,9-difluoro-1-(methylamino)-6-oxo-1,4,5,6-tetrahydrobenzo[c][1,7]naphthyridine-3(2H)-carboxylate (Vg) and 4-chloro-3,5-difluorobenzoic acid (VIcq).

Enantiomer I (Compound 168): LCMS: m/z found 440.2 [M+H]⁺, RT=5.02 min, (Method A); ¹H NMR (400 MHz, DMSO-d₆) δ 11.61 (s, 1H), 8.13-8.03 (m, 1H), 7.41 (d, 2H), 7.33-7.28 (m, 1H), 5.52 (s, 1H), 3.76 (d, 1H), 3.65 (d, 1H), 3.32-3.27 (d, 1H), 3.12-3.08 (m, 1H), 2.80-2.76 (m, 1H), 2.72 (s, 3H); Chiral analytical SFC: RT=2.62 min, Column: Chiralcel OX-3 (4.6×150 mm) 3 μm, 30% (0.5% DEA in Methanol), Flow rate: 3.0 g/min.

Enantiomer II (Compound 169): LCMS: m/z found 440.2 [M+H]⁺, RT=5.02 min, (Method A); ¹H NMR (400 MHz, DMSO-d₆) δ 11.61 (s, 1H), 8.13-8.03 (m, 1H), 7.41 (d, 2H), 7.33-7.28 (m, 1H), 5.52 (s, 1H), 3.76 (d, 1H), 3.65 (d, 1H), 3.32-3.27 (d, 1H), 3.12-3.08 (m, 1H), 2.80-2.76 (m, 1H), 2.72 (s, 3H); Chiral analytical SFC: RT=3.15 min, Column: Chiralcel OX-3 (4.6×150 mm) 3 μm, 30% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-4-(difluoromethyl)-3,5-difluoro-N-methylbenzamide (Compounds 183 and 184)

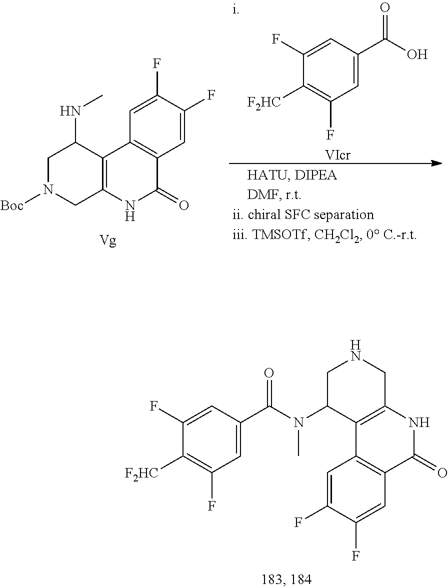

Individual enantiomers of N-(8,9-difluoro-6-oxo-1,2,3,4,5,6-hexahydrobenzo[c][1,7]naphthyridin-1-yl)-4-(difluoromethyl)-3,5-difluoro-N-methylbenzamide were prepared in an analogous manner as described above (for Compounds 160, 161), from racemic tert-butyl 8,9-difluoro-1-(methylamino)-6-oxo-1,4,5,6-tetrahydrobenzo[c][1,7]naphthyridine-3(2H)-carboxylate (Vg) and 4-(difluoromethyl)-3,5-difluorobenzoic acid (VIcr).

Enantiomer I (Compound 183): LCMS: m/z found 456.3 [M+H]⁺, RT=6.45 min, (Method A); ¹H NMR (400 MHz, DMSO-d₆) δ 11.67 (s, 1H), 8.12 (t, 1H), 7.45-7.19 (m, 4H), 5.60 (br s, 1H), 3.87 (d, 1H), 3.72 (d, 1H), 3.21-3.19 (m, 2H), 2.70 (m, 1H), 2.68 (s, 3H); Chiral analytical SFC: RT=1.75 min, Column: Chiralcel OX-3 (4.6×150 mm) 3 μm, 30% (0.5% DEA in Methanol), Flow rate: 3.0 g/min.

Enantiomer II (Compound 184): LCMS: m/z found 456.3 [M+H]⁺, RT=6.45 min, (Method A); ¹H NMR (400 MHz, DMSO-d₆) δ 11.67 (s, 1H), 8.12 (t, 1H), 7.45-7.19 (m, 4H), 5.60 (br s, 1H), 3.87 (d, 1H), 3.72 (d, 1H), 3.21-3.19 (m, 2H), 2.70 (m, 1H), 2.68 (s, 3H); Chiral analytical SFC: RT=2.09 min, Column: Chiralcel OX-3 (4.6×150 mm) 3 μm, 30% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-4-(difluoromethyl)-N-methyl-1H-indole-2-carboxamide (Compounds 207 and 208)

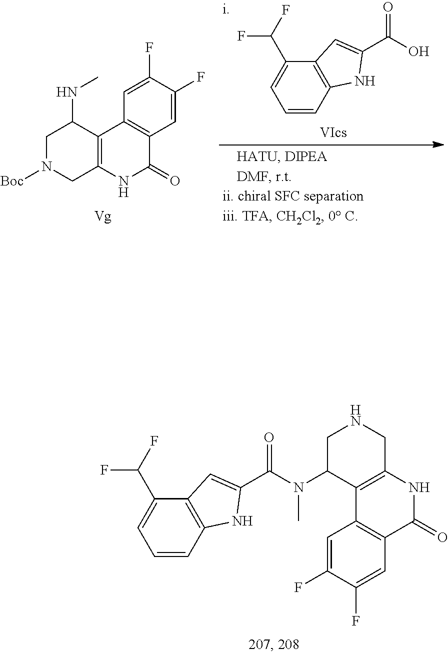 [SMILES: CNC1CN(C(=O)OC(C)(C)C)Cc2[nH]c(=O)c3cc(F)c(F)cc3c21 ; O=C(O)c1cc2c(C(F)F)cccc2[nH]1 ; CN(C(=O)c1cc2c(C(F)F)cccc2[nH]1)C1CNCc2[nH]c(=O)c3cc(F)c(F)cc3c21]

Individual enantiomers of N-(8,9-difluoro-6-oxo-1,2,3,4,5,6-hexahydrobenzo[c][1,7]naphthyridin-1-yl)-4-(difluoromethyl)-N-methyl-1H-indole-2-carboxamide were prepared in an analogous manner as described above (for Compounds 160, 161), from racemic tert-butyl 8,9-difluoro-1-(methylamino)-6-oxo-1,4,5,6-tetrahydrobenzo[c][1,7]naphthyridine-3(2H)-carboxylate (Vg) and 4-(difluoromethyl)-1H-indole-2-carboxylic acid (VIcs), except that separated isomers of intermediate tert-butyl 1-(4-(difluoromethyl)-N-methyl-1H-indole-2-carboxamido)-8,9-difluoro-6-oxo-1,4,5,6-tetrahydrobenzo[c][1,7]naphthyridine-3(2H)-carboxylate were treated with TFA (5.0 eq.) in DCM at 0° C., instead of TMSOTf, for the final, deprotection step.

Enantiomer I (Compound 207): LCMS: m/z found 459.3 [M+H]⁺, RT=5.07 min, (Method A); ¹H NMR (400 MHz, DMSO-d₆) δ 12.07 (s, 1H), 11.64 (bs, 1H), 8.11 (t, 1H), 7.64 (d, 1H), 7.41-7.14 (m, 4H), 7.00 (s, 1H), 5.68 (s, 1H), 3.81 (d, 1H), 3.66 (d, 1H), 3.15 (m, 5H); Chiral analytical SFC: RT=4.34 min, Column: Chiralcel OJ-3 (4.6×150 mm) 3 m, 15% (0.5% DEA in Methanol), Flow rate: 3.0 g/min.

Enantiomer I (Compound 208): LCMS: m/z found 459.3 [M+H]⁺, RT=5.07 min, (Method A); ¹H NMR (400 MHz, DMSO-d₆) δ 12.07 (s, 1H), 11.64 (bs, 1H), 8.11 (t, 1H), 7.64 (d, 1H), 7.41-7.14 (m, 4H), 7.00 (s, 1H), 5.68 (s, 1H), 3.81 (d, 1H), 3.66 (d, 1H), 3.15 (m, 5H); Chiral analytical SFC: RT=5.25 min, Column: Chiralcel OJ-3 (4.6×150 mm) 3 m, 15% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-6-(difluoromethyl)-5-fluoro-N-methyl-1H-indole-2-carboxamide (Compounds 220 and 221)

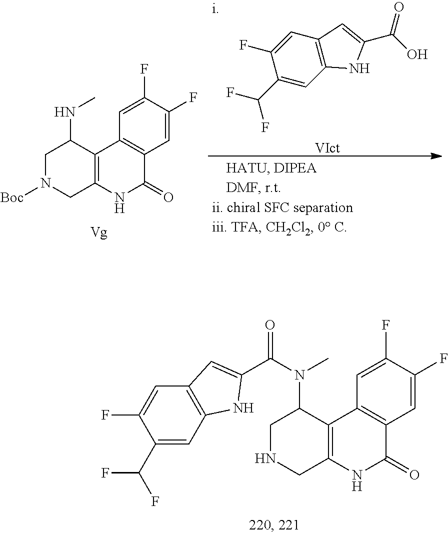

Individual enantiomers of N-(8,9-difluoro-6-oxo-1,2,3,4,5,6-hexahydrobenzo[c][1,7]naphthyridin-1-yl)-6-(difluoromethyl)-5-fluoro-N-methyl-1H-indole-2-carboxamide were prepared in an analogous manner as described above (for Compounds 207, 208), from racemic tert-butyl 8,9-difluoro-1-(methylamino)-6-oxo-1,4,5,6-tetrahydrobenzo[c][1,7]naphthyridine-3(2H)-carboxylate (Vg) and 6-(difluoromethyl)-5-fluoro-1H-indole-2-carboxylic acid (VIct).

Enantiomer I (Compound 220): LCMS: m/z found 477.3 [M+H]⁺, RT=5.09 min, (Method A); ¹H NMR (400 MHz, DMSO-d₆) δ 12.14 (bs, 1H), 11.65 (bs, 1H), 8.13-8.08 (m, 1H), 7.69 (d, 1H), 7.53 (d, 1H), 7.39-7.11 (m, 2H), 6.95 (s, 1H), 5.67 (s, 1H), 3.81 (d, 1H), 3.66 (d, 1H), 3.14-2.78 (m, 6H); Chiral analytical SFC: RT=3.97 min, Column: Chiralcel OD-3 (4.6×150 mm) 3 μm, 25% (0.5% DEA in Methanol), Flow rate: 3.0 g/min.

Enantiomer II (Compound 221): LCMS: m/z found 477.3 [M+H]⁺, RT=5.09 min, (Method A); ¹H NMR (400 MHz, DMSO-d₆) δ 12.14 (bs, 1H), 11.65 (bs, 1H), 8.13-8.08 (m, 1H), 7.69 (d, 1H), 7.53 (d, 1H), 7.39-7.11 (m, 2H), 6.95 (s, 1H), 5.67 (s, 1H), 3.81 (d, 1H), 3.66 (d, 1H), 3.14-2.78 (m, 6H); Chiral analytical SFC: RT=5.12 min, Column: Chiralcel OD-3 (4.6×150 mm) 3 μm, 25% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-6-fluoro-N-methylindolizine-2-carboxamide (Compounds 222 and 223)

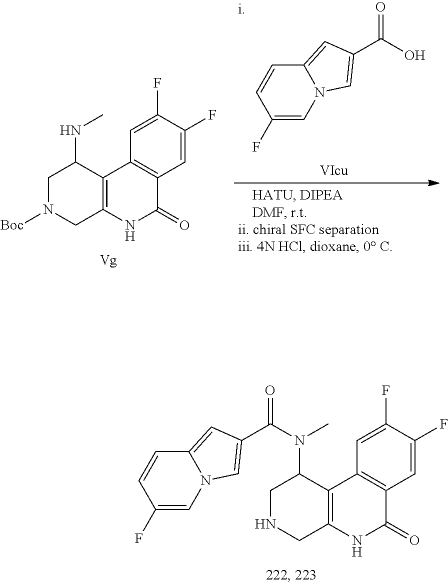 [SMILES: CNC1CN(C(=O)OC(C)(C)C)Cc2[nH]c(=O)c3cc(F)c(F)cc3c21 ; O=C(O)c1cc2ccc(F)cn2c1 ; CN(C(=O)c1cc2ccc(F)cn2c1)C1CNCc2[nH]c(=O)c3cc(F)c(F)cc3c21]

Individual enantiomers of N-(8,9-difluoro-6-oxo-1,2,3,4,5,6-hexahydrobenzo[c][1,7]naphthyridin-1-yl)-6-fluoro-N-methylindolizine-2-carboxamide were prepared in an analogous manner as described above (for Compounds 160, 161), from racemic tert-butyl 8,9-difluoro-1-(methylamino)-6-oxo-1,4,5,6-tetrahydrobenzo[c][1,7]naphthyridine-3(2H)-carboxylate (Vg) and 6-fluoroindolizine-2-carboxylic acid (VIcu), except that separated isomers of intermediate tert-butyl 8,9-difluoro-1-(6-fluoro-N-methylindolizine-2-carboxamido)-6-oxo-1,4,5,6-tetrahydrobenzo[c][1,7]naphthyridine-3(2H)-carboxylate were treated with HCl 4N in dioxane (5.0 eq.) at 0° C., instead of TMSOTf, for the final step.

Enantiomer I (Compound 222): LCMS: m/z found 427.3 [M+H]⁺, RT=4.09 min, (Method A); ¹H NMR (400 MHz, DMSO-d₆) δ 11.61 (bs, 1H), 8.45 (bs, 1H), 8.10 (t, 1H), 7.91 (s, 1H), 7.54-7.50 (m, 1H), 7.32-7.27 (m, 1H), 6.85-6.73 (m, 2H), 5.63 (s, 1H), 3.77 (d, 1H), 3.63 (d, 1H), 3.15 (s, 2H), 2.99 (s, 4H); Chiral analytical SFC: RT=4.04 min, Column: Chiralcel OD-3 (4.6×150 mm) 3 μm, 25% (0.5% DEA in Methanol), Flow rate: 3.0 g/min.

Enantiomer II (Compound 223): LCMS: m/z found 427.3 [M+H]⁺, RT=4.09 min, (Method A); ¹H NMR (400 MHz, DMSO-d₆) δ 11.61 (bs, 1H), 8.45 (bs, 1H), 8.10 (t, 1H), 7.91 (s, 1H), 7.54-7.50 (m, 1H), 7.32-7.27 (m, 1H), 6.85-6.73 (m, 2H), 5.63 (s, 1H), 3.77 (d, 1H), 3.63 (d, 1H), 3.15 (s, 2H), 2.99 (s, 4H); Chiral analytical SFC: RT=5.31 min, Column: Chiralcel OD-3 (4.6×150 mm) 3 μm, 25% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-4-(difluoromethyl)-6-fluoro-N-methyl-1H-indole-2-carboxamide (Compounds 257 and 258)

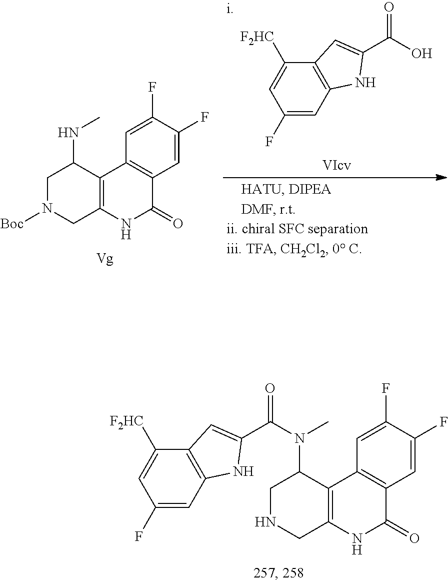 [SMILES: CNC1CN(C(=O)OC(C)(C)C)Cc2[nH]c(=O)c3cc(F)c(F)cc3c21 ; O=C(O)c1cc2c(C(F)F)cc(F)cc2[nH]1 ; CN(C(=O)c1cc2c(C(F)F)cc(F)cc2[nH]1)C1CNCc2[nH]c(=O)c3cc(F)c(F)cc3c21]

Individual enantiomers of N-(8,9-difluoro-6-oxo-1,2,3,4,5,6-hexahydrobenzo[c][1,7]naphthyridin-1-yl)-4-(difluoromethyl)-6-fluoro-N-methyl-1H-indole-2-carboxamide were prepared in an analogous manner as described above (for Compounds 207, 208), from racemic tert-butyl 8,9-difluoro-1-(methylamino)-6-oxo-1,4,5,6-tetrahydrobenzo[c][1,7]naphthyridine-3(2H)-carboxylate (Vg) and 4-(difluoromethyl)-6-fluoro-1H-indole-2-carboxylic acid (VIcv).

Enantiomer I (Compound 257): LCMS: m/z found 477.4 [M+H]⁺, RT=6.29 min, (Method A); ¹H NMR (400 MHz, DMSO-d₆) δ 12.15 (bs, 1H), 11.68 (bs, 1H), 8.11 (m, 1H), 7.45-7.18 (m, 4H), 7.03 (s, 1H), 5.67 (s, 1H), 3.81 (d, 1H), 3.66 (d, 1H), 3.14-2.93 (m, 5H), 2.7 (s, 1H); Chiral analytical SFC: RT=2.29 min, Column: Chiralcel AS-3 (4.6×150 mm) 3 m, 20% (0.5% DEA in Methanol), Flow rate: 3.0 g/min.

Enantiomer II (Compound 258): LCMS: m/z found 477.4 [M+H]⁺, RT=6.29 min, (Method A); ¹H NMR (400 MHz, DMSO-d₆) δ 12.15 (bs, 1H), 11.68 (bs, 1H), 8.11 (m, 1H), 7.45-7.18 (m, 4H), 7.03 (s, 1H), 5.67 (s, 1H), 3.81 (d, 1H), 3.66 (d, 1H), 3.14-2.93 (m, 5H), 2.7 (s, 1H); Chiral analytical SFC: RT=3.76 min, Column: Chiralcel AS-3 (4.6×150 mm) 3 m, 20% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-4-(difluoromethyl)-5-fluoro-N-methyl-1H-indole-2-carboxamide (Compounds 264 and 265)

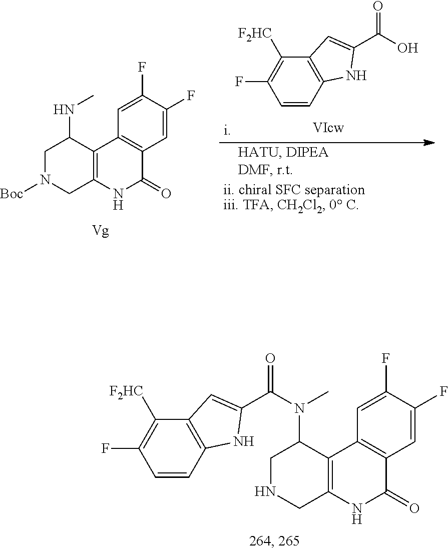 [SMILES: CNC1CN(C(=O)OC(C)(C)C)Cc2[nH]c(=O)c3cc(F)c(F)cc3c21 ; "ii. chiral SFC separationiii. TFA, CH2Cl2, 0° C." ; O=C(O)c1cc2c(C(F)F)c(F)ccc2[nH]1 ; CN(C(=O)c1cc2c(C(F)F)c(F)ccc2[nH]1)C1CNCc2[nH]c(=O)c3cc(F)c(F)cc3c21]

Individual enantiomers of N-(8,9-difluoro-6-oxo-1,2,3,4,5,6-hexahydrobenzo[c][1,7]naphthyridin-1-yl)-4-(difluoromethyl)-5-fluoro-N-methyl-1H-indole-2-carboxamide were prepared in an analogous manner as described above (for Compounds 207, 208), from racemic tert-butyl 8,9-difluoro-1-(methylamino)-6-oxo-1,4,5,6-tetrahydrobenzo[c][1,7]naphthyridine-3(2H)-carboxylate (Vg) and 4-(difluoromethyl)-5-fluoro-1H-indole-2-carboxylic acid (VIcw).

Enantiomer I (Compound 264): LCMS: m/z found 477.3 [M+H]⁺, RT=7.20 min, (Method A); ¹H NMR (400 MHz, DMSO-d₆) δ 12.17 (bs, 1H), 11.65 (bs, 1H), 8.11 (t, 1H), 7.66-7.27 (m, 3H), 7.19 (t, 1H), 7.03 (s, 1H), 5.67 (s, 1H), 3.81 (d, 1H), 3.69 (d, 1H), 3.23-3.13 (m, 5H), 2.79 (s, 1H); Chiral analytical SFC: RT=1.51 min, Column: Chiralcel AS-3 (4.6×150 mm) 3 μm, 30% (0.5% DEA in Methanol), Flow rate: 3.0 g/min.

Enantiomer II (Compound 265): LCMS: m/z found 477.3 [M+H]⁺, RT=7.20 min, (Method A); ¹H NMR (400 MHz, DMSO-d₆) δ 12.17 (bs, 1H), 11.65 (bs, 1H), 8.11 (t, 1H), 7.66-7.27 (m, 3H), 7.19 (t, 1H), 7.03 (s, 1H), 5.67 (s, 1H), 3.81 (d, 1H), 3.69 (d, 1H), 3.23-3.13 (m, 5H), 2.79 (s, 1H); Chiral analytical SFC: RT=2.82 min, Column: Chiralcel AS-3 (4.6×150 mm) 3 μm, 30% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-3′-fluoro-N-methyl-[1,1′-biphenyl]-4-carboxamide (Compounds 301 and 302)

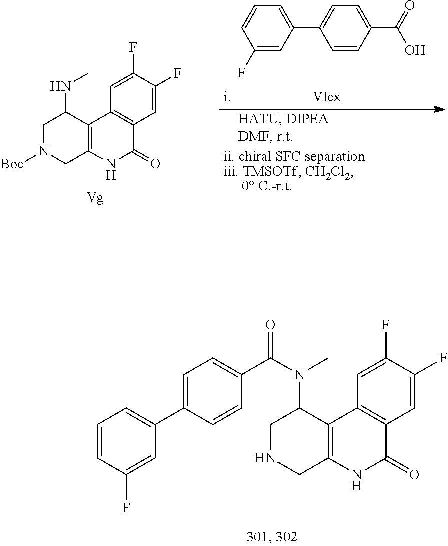 [SMILES: CNC1CN(C(=O)OC(C)(C)C)Cc2[nH]c(=O)c3cc(F)c(F)cc3c21 ; "ii. chiral SFC separationiii. TMSOTf, CH2Cl2,     0° C.-r.t." ; O=C(O)c1ccc(-c2cccc(F)c2)cc1 ; CN(C(=O)c1ccc(-c2cccc(F)c2)cc1)C1CNCc2[nH]c(=O)c3cc(F)c(F)cc3c21]

Individual enantiomers of N-(8,9-difluoro-6-oxo-1,2,3,4,5,6-hexahydrobenzo[c][1,7]naphthyridin-1-yl)-3′-fluoro-N-methyl-[1,1′-biphenyl]-4-carboxamide were prepared in an analogous manner as described above (for Compounds 160, 161), from racemic tert-butyl 8,9-difluoro-1-(methylamino)-6-oxo-1,4,5,6-tetrahydrobenzo[c][1,7]naphthyridine-3(2H)-carboxylate (Vg) and 3′-fluoro-[1,1′-biphenyl]-4-carboxylic acid (VIcx).

Enantiomer I (Compound 301): LCMS: m/z found 464.2 [M+H]⁺, RT=3.79 min, (Method A); ¹H NMR (400 MHz, DMSO-d₆) δ 11.62 (bs, 1H), 8.12 (t, 2H), 7.79 (d, 2H), 7.57-7.35 (m, 6H), 7.23 (t, 1H), 5.62 (s, 1H), 3.77 (d, 1H), 3.65 (d, 1H), 3.32-3.15 (m, 2H), 2.75 (s, 3H); Chiral analytical SFC: RT=3.81 min, Column: LUX cellulose (4.6×150 mm) 3 μm, 30% Methanol, Flow rate: 3.0 g/min.

Enantiomer II (Compound 302): LCMS: m/z found 464.2 [M+H]⁺, RT=3.79 min, (Method A); ¹H NMR (400 MHz, DMSO-d₆) δ 11.62 (bs, 1H), 8.12 (t, 2H), 7.79 (d, 2H), 7.57-7.35 (m, 6H), 7.23 (t, 1H), 5.62 (s, 1H), 3.77 (d, 1H), 3.65 (d, 1H), 3.32-3.15 (m, 2H), 2.75 (s, 3H); Chiral analytical SFC: RT=6.45 min, Column: LUX cellulose (4.6×150 mm) 3 μm, 30% Methanol, Flow rate: 3.0 g/min.

### N-(8,9-Difluoro-6-oxo-1,2,3,4,5,6-hexahydrobenzo[c][1,7]naphthyridin-1-yl)-3′-fluoro-N-methyl-[1,1′-biphenyl]-3-carboxamide (Compounds 304 and 305)

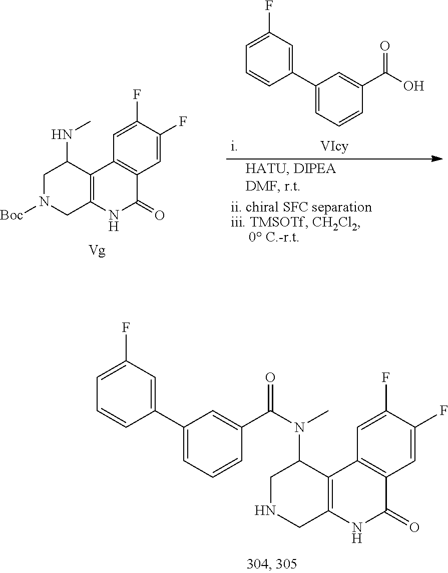 [SMILES: CNC1CN(C(=O)OC(C)(C)C)Cc2[nH]c(=O)c3cc(F)c(F)cc3c21 ; "ii. chiral SFC separationiii. TMSOTf, CH2Cl2,     0° C.-r.t." ; O=C(O)c1cccc(-c2cccc(F)c2)c1 ; CN(C(=O)c1cccc(-c2cccc(F)c2)c1)C1CNCc2[nH]c(=O)c3cc(F)c(F)cc3c21]

Individual enantiomers of N-(8,9-difluoro-6-oxo-1,2,3,4,5,6-hexahydrobenzo[c][1,7]naphthyridin-1-yl)-3′-fluoro-N-methyl-[1,1′-biphenyl]-3-carboxamide were prepared in an analogous manner as described above (for Compounds 160, 161), from racemic tert-butyl 8,9-difluoro-1-(methylamino)-6-oxo-1,4,5,6-tetrahydrobenzo[c][1,7]naphthyridine-3(2H)-carboxylate (Vg) and 3′-fluoro-[1,1′-biphenyl]-3-carboxylic acid (VIcy).

Enantiomer I (Compound 304): LCMS: m/z found 464.2 [M+H]⁺, RT=3.71 min, (Method A); ¹H NMR (400 MHz, DMSO-d₆) δ 11.62 (bs, 1H), 8.12 (t, 1H), 7.78 (d, 1H), 7.68 (s, 1H), 7.58-7.49 (m, 4H), 7.43-7.37 (m, 2H), 7.23 (t, 1H), 5.63 (s, 1H), 3.77 (d, 1H), 3.65 (d, 1H), 3.18-3.14 (m, 2H), 2.83-2.73 (m, 4H); Chiral analytical SFC: RT=6.06 min, Column: Chiralcel OD-3 (4.6×150 mm) 3 μm, 20% (0.5% DEA in Methanol), Flow rate: 3.0 g/min.

Enantiomer II (Compound 305): LCMS: m/z found 464.2 [M+H]⁺, RT=3.71 min, (Method A); ¹H NMR (400 MHz, DMSO-d₆) δ 11.62 (bs, 1H), 8.12 (t, 1H), 7.78 (d, 1H), 7.68 (s, 1H), 7.58-7.49 (m, 4H), 7.43-7.37 (m, 2H), 7.23 (t, 1H), 5.63 (s, 1H), 3.77 (d, 1H), 3.65 (d, 1H), 3.18-3.14 (m, 2H), 2.83-2.73 (m, 4H); Chiral analytical SFC: RT=7.44 min, Column: Chiralcel OD-3 (4.6×150 mm) 3 μm, 20% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-N-methyl-3-(N-methylsulfamoyl)benzamide (Compounds 318 and 319)

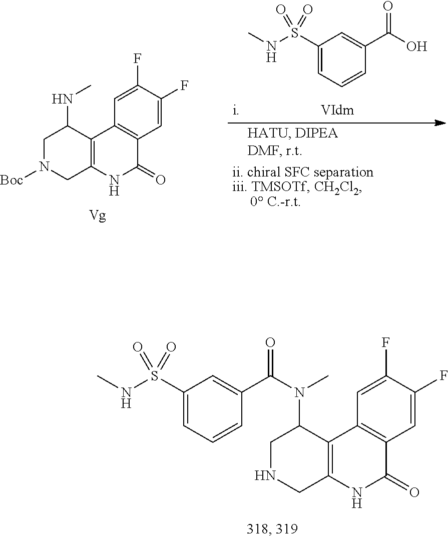 [SMILES: CNC1CN(C(=O)OC(C)(C)C)Cc2[nH]c(=O)c3cc(F)c(F)cc3c21 ; "ii. chiral SFC separationiii. TMSOTf, CH2Cl2,     0° C.-r.t." ; CNS(=O)(=O)c1cccc(C(=O)O)c1 ; CNS(=O)(=O)c1cccc(C(=O)N(C)C2CNCc3[nH]c(=O)c4cc(F)c(F)cc4c32)c1]

Individual enantiomers of N-(8,9-difluoro-6-oxo-1,2,3,4,5,6-hexahydrobenzo[c][1,7]naphthyridin-1-yl)-N-methyl-3-(N-methylsulfamoyl)benzamide were prepared in an analogous manner as described, from racemic tert-butyl 8,9-difluoro-1-(methylamino)-6-oxo-1,4,5,6-tetrahydrobenzo[c][1,7]naphthyridine-3(2H)-carboxylate (Vg) and 3-(N-methylsulfamoyl)benzoic acid (VIdm).

Enantiomer I (Compound 319): LCMS: m/z found 463.1 [M+H]⁺, RT=2.21 min, (Method A); ¹H NMR (400 MHz, DMSO-d₆) δ 11.63 (bs, 1H), 8.12 (t, 1H), 7.84 (d, 1H), 7.76-7.64 (m, 4H), 7.38-7.33 (m, 1H), 5.60 (s, 1H), 3.77 (d, 1H), 3.65 (d, 1H), 3.28-3.12 (m, 2H), 2.81-2.71 (m, 4H), 2.42 (d, 3H); Chiral analytical SFC: RT=4.63 min, Column: (R,R)Whelk 01 (4.6×150 mm) 3 μm, 30% (0.5% DEA in Methanol), Flow rate: 3.0 g/min.

Enantiomer II (Compound 318): LCMS: m/z found 463.1 [M+H]⁺, RT=2.21 min, (Method A); ¹H NMR (400 MHz, DMSO-d₆) δ 11.63 (bs, 1H), 8.12 (t, 1H), 7.84 (d, 1H), 7.76-7.64 (m, 4H), 7.38-7.33 (m, 1H), 5.60 (s, 1H), 3.77 (d, 1H), 3.65 (d, 1H), 3.28-3.12 (m, 2H), 2.81-2.71 (m, 4H), 2.42 (d, 3H); Chiral analytical SFC: RT=5.25 min, Column: (R,R)Whelk 01 (4.6×150 mm) 3 μm, 30% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-3-(3-fluorophenoxy)-N-methylbenzamide (Compounds 339 and 340)

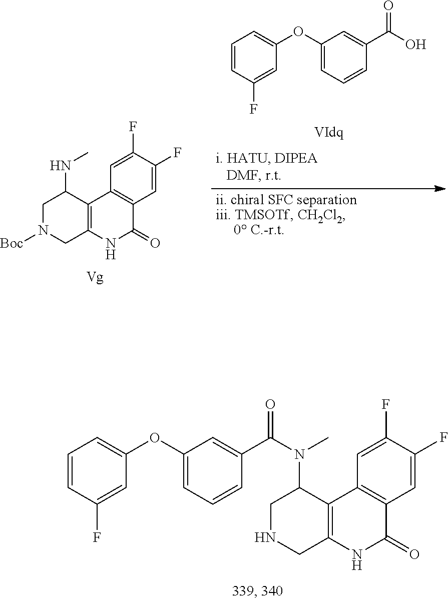 [SMILES: O=C(O)c1cccc(Oc2cccc(F)c2)c1 ; CNC1CN(C(=O)OC(C)(C)C)Cc2[nH]c(=O)c3cc(F)c(F)cc3c21 ; "ii. chiral SFC separationiii. TMSOTf, CH2Cl2,     0° C.-r.t." ; CN(C(=O)c1cccc(Oc2cccc(F)c2)c1)C1CNCc2[nH]c(=O)c3cc(F)c(F)cc3c21]

Individual enantiomers of N-(8,9-difluoro-6-oxo-1,2,3,4,5,6-hexahydrobenzo[c][1,7]naphthyridin-1-yl)-3-(3-fluorophenoxy)-N-methylbenzamide were prepared in an analogous manner as described, from racemic tert-butyl 8,9-difluoro-1-(methylamino)-6-oxo-1,4,5,6-tetrahydrobenzo[c][1,7]naphthyridine-3(2H)-carboxylate (Vg) and 3-(3-fluorophenoxy)benzoic acid (VIdq).

Enantiomer I (Compound 339): LCMS: m/z found 480.2 [M+H]⁺, RT=3.70 min, (Method A); ¹H NMR (400 MHz, DMSO-d₆) δ 11.60 (s, 1H), 8.12-8.07 (m, 1H), 7.51-7.39 (m, 2H), 7.30-7.25 (m, 1H), 7.16-7.15 (m, 2H), 7.02-6.85 (m, 4H), 5.55 (s, 1H), 3.77-3.60 (m, 2H), 3.24-3.1101 (m, 2H), 2.85-2.64 (m, 4H); Chiral analytical SFC: RT=2.48 min, Column: Chiralpak IG-3 (4.6×150 mm) 3 μm, 40% (0.5% DEA in Methanol), Flow rate: 3.0 g/min.

Enantiomer II (Compound 340): LCMS: m/z found 480.2 [M+H]⁺, RT=3.70 min, (Method A); ¹H NMR (400 MHz, DMSO-d₆) δ 11.60 (s, 1H), 8.12-8.07 (m, 1H), 7.51-7.39 (m, 2H), 7.30-7.25 (m, 1H), 7.16-7.15 (m, 2H), 7.02-6.85 (m, 4H), 5.55 (s, 1H), 3.77-3.60 (m, 2H), 3.24-3.11 (m, 2H), 2.85-2.64 (m, 4H); Chiral analytical SFC: RT=4.69 min, Column: Chiralpak IG-3 (4.6×150 mm) 3 μm, 40% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-N-methylindolizine-2-carboxamide (Compounds 506 and 507)

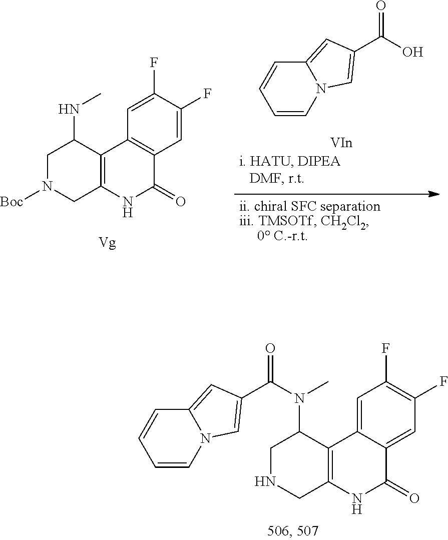

Individual enantiomers of N-(8,9-difluoro-6-oxo-1,2,3,4,5,6-hexahydrobenzo[c][1,7]naphthyridin-1-yl)-N-methylindolizine-2-carboxamide were prepared in an analogous manner as described, from racemic tert-butyl 8,9-difluoro-1-(methylamino)-6-oxo-1,4,5,6-tetrahydrobenzo[c][1,7]naphthyridine-3(2H)-carboxylate (Vg) and indolizine-2-carboxylic acid (VIn).

Enantiomer I (Compound 506): LCMS: m/z found 409.2 [M+H]⁺, RT=5.57 min, (Method A); ¹H NMR (400 MHz, DMSO-d₆) δ 11.61 (br s, 1H), 8.24 (d, 1H), 8.12-8.08 (m, 1H), 7.89 (m, 1H), 7.42 (m, 1H), 7.33-7.01 (m, 1H), 6.75-6.72 (m, 3H), 5.64-5.23 (s, 1H), 3.78 (d, 1H), 3.65 (d, 1H), 3.15 (m, 2H), 3.01-2.77 (m, 4H); Chiral analytical SFC: RT=1.27 min, Column: Chiralcel OJ-3 (4.6×150 mm) 3 μm, 30% (0.5% DEA in Methanol), Flow rate: 3.0 g/min.

Enantiomer II (Compound 507): LCMS: m/z found 409.2 [M+H]⁺, RT=5.57 min, (Method A); ¹H NMR (400 MHz, DMSO-d₆) δ 11.61 (br s, 1H), 8.24 (d, 1H), 8.12-8.08 (m, 1H), 7.89 (m, 1H), 7.42 (m, 1H), 7.33-7.01 (m, 1H), 6.75-6.72 (m, 3H), 5.64-5.23 (s, 1H), 3.78 (d, 1H), 3.65 (d, 1H), 3.15 (m, 2H), 3.01-2.77 (m, 4H); Chiral analytical SFC: RT=1.75 min, Column: Chiralcel OJ-3 (4.6×150 mm) 3 μm, 30% (0.5% DEA in Methanol), Flow rate: 3.0 g/min.

### 8-Chloro-N-(8,9-Difluoro-6-oxo-1,2,3,4,5,6-hexahydrobenzo[c][1,7]naphthyridin-1-yl)-N-methylindolizine-2-carboxamide (Compounds 508 and 509)

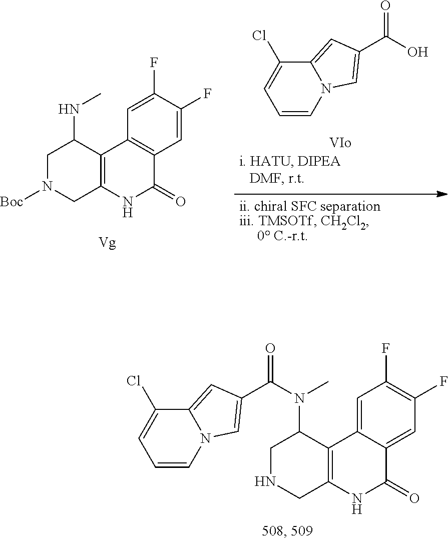

Individual enantiomers of 8-chloro-N-(8,9-difluoro-6-oxo-1,2,3,4,5,6-hexahydrobenzo[c][1,7]naphthyridin-1-yl)-N-methylindolizine-2-carboxamide were prepared in an analogous manner as described, from racemic tert-butyl 8,9-difluoro-1-(methylamino)-6-oxo-1,4,5,6-tetrahydrobenzo[c][1,7]naphthyridine-3(2H)-carboxylate (Vg) and 8-chloroindolizine-2-carboxylic acid (VIo).

Enantiomer I (Compound 508): LCMS: m/z found 443.2 [M+H]⁺, RT=6.02 min, (Method A); ¹H NMR (400 MHz, DMSO-d₆) δ 11.62 (br s, 1H), 8.28 (d, 1H), 8.13-8.02 (m, 2H), 7.33-7.28 (m, 1H), 6.96 (d, 1H), 6.72 (s, 1H), 6.64 (t, 1H), 5.64 (s, 1H), 3.78 (d, 1H), 3.64 (d, 1H), 3.16 (m, 2H), 3.01-2.76 (m, 4H); Chiral analytical SFC: RT=1.29 min, Column: Chiralcel OJ-3 (4.6×150 mm) 3 μm, 30% (0.5% DEA in Methanol), Flow rate: 3.0 g/min.

Enantiomer II (Compound 509): LCMS: m/z found 443.2 [M+H]⁺, RT=6.02 min, (Method A); ¹H NMR (400 MHz, DMSO-d₆) δ 11.62 (br s, 1H), 8.28 (d, 1H), 8.13-8.02 (m, 2H), 7.33-7.28 (m, 1H), 6.96 (d, 1H), 6.72 (s, 1H), 6.64 (t, 1H), 5.64 (s, 1H), 3.78 (d, 1H), 3.64 (d, 1H), 3.16 (m, 2H), 3.01-2.76 (m, 4H); Chiral analytical SFC: RT=1.58 min, Column: Chiralcel OJ-3 (4.6×150 mm) 3 μm, 30% (0.5% DEA in Methanol), Flow rate: 3.0 g/min.

### N-(8,9-Difluoro-3-methyl-6-oxo-1,2,3,4,5,6-hexahydrobenzo[c][1,7]naphthyridin-1-yl)-N-methyl-1H-indole-2-carboxamide (Compounds 62 and 63)

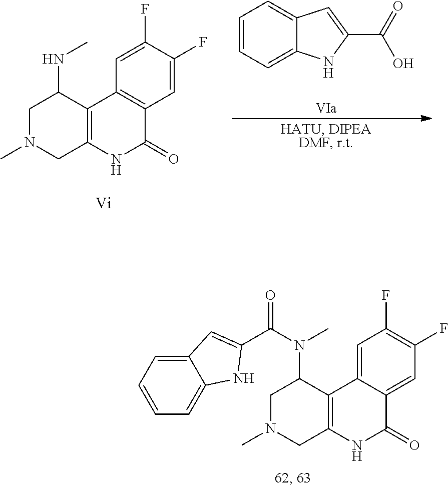

Racemic N-(8,9-difluoro-3-methyl-6-oxo-1,2,3,4,5,6-hexahydrobenzo[c][1,7]naphthyridine-1-yl)-N-methyl-1H-indole-2-carboxamide was synthesized in an analogous manner as described above, from racemic 8,9-difluoro-3-methyl-1-(methylamino)-1,3,4,5-tetrahydrobenzo[c][1,7]naphthyridin-6(2H)-one (Vi) and 1H-indole-2-carboxylic acid (VIa).

The enantiomers were subsequently separated by preparative SFC: Method isocratic, Mobile phase methanol:CO₂—40:60. Column: Chiralpak IC (30×250 mm), 5 μm, flow rate: 110 g/min.

Enantiomer I (Compound 62): LCMS: m/z found 423.2 [M+H]⁺, RT=3.20 min, (Method A); ¹H NMR (400 MHz, DMSO-d₆) δ 11.69 (br s, 2H), 8.13-8.08 (m, 1H) 7.62 (d, 1H), 7.47 (d, 1H), 7.32-7.29 (m, 1H), 7.21 (t, 1H), 7.04 (t, 1H), 6.90 (s, 1H), 5.82 (s, 1H), 3.72 (d, 1H), 3.16 (s, 3H), 3.08-2.9 (m, 2H), 2.73-2.69 (m, 1H), 2.32 (s, 3H); Chiral analytical SFC: RT=2.92 min, Column: Chiralpak IC-3 (4.6×150 mm) 3 μm, 40% of (0.5% DEA in Methanol), Flow rate: 3.0 g/min.

Enantiomer II (Compound 63): LCMS: m/z found 423.2 [M+H]⁺, RT=3.20 min, (Method A); ¹H NMR (400 MHz, DMSO-d₆) δ 11.69 (br s, 2H), 8.13-8.08 (m, 1H) 7.62 (d, 1H), 7.47 (d, 1H), 7.32-7.29 (m, 1H), 7.21 (t, 1H), 7.04 (t, 1H), 6.90 (s, 1H), 5.82 (s, 1H), 3.72 (d, 1H), 3.16 (s, 3H), 3.08-2.9 (m, 2H), 2.73-2.69 (m, 1H), 2.32 (s, 3H); Chiral analytical SFC: RT=5.78 min, Column: Chiralpak IC-3 (4.6×150 mm) 3 μm, 40% of (0.5% DEA in Methanol), Flow rate: 3.0 g/min.

### N-(3-Acetyl-8,9-difluoro-6-oxo-1,2,3,4,5,6-hexahydrobenzo[c][1,7]naphthyridin-1-yl)-N-methyl-1H-indole-2-carboxamide (Compounds 81 and 82)

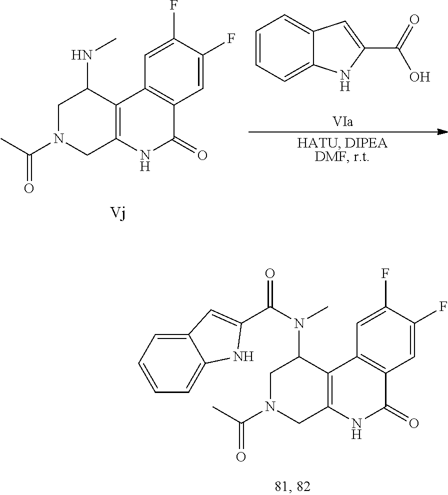

Racemic N-(3-acetyl-8,9-difluoro-6-oxo-1,2,3,4,5,6-hexahydrobenzo[c][1,7]naphthyridin-1-yl)-N-methyl-1H-indole-2-carboxamide was synthesized in an analogous manner as described above, from racemic 3-acetyl-8,9-difluoro-1-(methylamino)-1,3,4,5-tetrahydrobenzo[c][1,7]naphthyridin-6(2H)-one (Vj) and 1H-indole-2-carboxylic acid (VIa).

The enantiomers were subsequently separated by preparative SFC: Method isocratic, Mobile phase methanol:CO₂—50:50. Column: Chiralpak IA (30×250 mm), 5 μm, flow rate: 120 g/min.

Enantiomer I (Compound 81: LCMS: m/z found 451.2 [M+H]⁺, RT=4.15 min, (Method A); ¹H NMR (400 MHz, DMSO-d₆) δ 11.82 (br s, 2H), 8.12 (t, 1H) 7.61 (dd, 1H), 7.48 (d, 1H), 7.41-7.32 (m, 1H), 7.22 (t, 1H), 7.05 (t, 1H), 6.99 (s, 1H), 5.92-5.86 (s, 1H), 5.08 (d, 1H), 4.67 (d, 1H), 3.99 (d, 1H), 3.72 (d, 1H), 2.97 (s, 3H), 2.13 (s, 3H); Chiral analytical SFC: RT=3.01 min, Column: Chiralpak IA-3 (4.6×150 mm) 3 μm, 50% of MeOH, Flow rate: 3.0 g/min.

Enantiomer II (Compound 82): LCMS: m/z found 451.2 [M+H]⁺, RT=4.15 min, (Method A); ¹H NMR (400 MHz, DMSO-d₆) δ 11.82 (br s, 2H), 8.12 (t, 1H) 7.61 (dd, 1H), 7.48 (d, 1H), 7.41-7.32 (m, 1H), 7.22 (t, 1H), 7.05 (t, 1H), 6.99 (s, 1H), 5.92-5.86 (s, 1H), 5.08 (d, 1H), 4.67 (d, 1H), 3.99 (d, 1H), 3.72 (d, 1H), 2.97 (s, 3H), 2.13 (s, 3H); Chiral analytical SFC: RT=6.43 min, Column: Chiralpak IA-3 (4.6×150 mm) 3 μm, 50% of MeOH, Flow rate: 3.0 g/min.

### N-(8,9-Difluoro-3-(2-hydroxyethyl)-6-oxo-1,2,3,4,5,6-hexahydrobenzo[c][1,7]naphthyridin-1-yl)-N-methyl-1H-indole-2-carboxamide (Compounds 89 and 90)

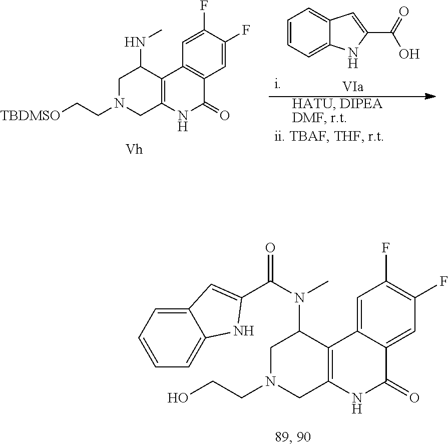

Racemic N-(8,9-difluoro-3-(2-hydroxyethyl)-6-oxo-1,2,3,4,5,6-hexahydrobenzo[c][1,7]naphthyridin-1-yl)-N-methyl-1H-indole-2-carboxamide was synthesized in an analogous manner as described above, from racemic 3-(2-((tert-butyldimethylsilyl)oxy)ethyl)-8,9-difluoro-1-(methylamino)-1,3,4,5-tetrahydrobenzo[c][1,7]naphthyridin-6(2H)-one (Vh) and 1H-indole-2-carboxylic acid (VIa). The enantiomers were subsequently separated by preparative SFC: Method isocratic, Mobile phase methanol:CO₂—50:50. Column: Chiralpak IC (30×250 mm), 5 μm, flow rate: 120 g/min.

Enantiomer I (Compound 89): LCMS: m/z found 453.2 [M+H]⁺, RT=4.06 min, (Method A); ¹H NMR (400 MHz, DMSO-d₆) δ 11.70 (br s, 2H), 8.11 (t, 1H), 7.60 (d, 1H), 7.47 (d, 1H), 7.33 (dd, 1H), 7.21 (t, 1H), 7.04 (t, 1H), 6.90 (s, 1H), 5.83 (s, 1H), 4.55 (s, 1H), 3.83 (d, 1H), 3.58 (d, 2H), 3.20 (s, 3H), 3.15 (m, 2H), 2.82 (m, 1H), 2.60 (m, 2H); Chiral analytical SFC: RT=2.05 min, Column: Chiralpak IC-3 (4.6×150 mm) 3 μm, 50% of Methanol, Flow rate: 3.0 g/min.

Enantiomer II (Compound 90): LCMS: m/z found 453.2 [M+H]⁺, RT=4.06 min, (Method A); ¹H NMR (400 MHz, DMSO-d₆) δ 11.70 (br s, 2H), 8.11 (t, 1H), 7.60 (d, 1H), 7.47 (d, 1H), 7.33 (dd, 1H), 7.21 (t, 1H), 7.04 (t, 1H), 6.90 (s, 1H), 5.83 (s, 1H), 4.55 (s, 1H), 3.83 (d, 1H), 3.58 (d, 2H), 3.20 (s, 3H), 3.15 (m, 2H), 2.82 (m, 1H), 2.60 (m, 2H); Chiral analytical SFC: RT=3.74 min, Column: Chiralpak IC-3 (4.6×150 mm) 3 μm, 50% of Methanol, Flow rate: 3.0 g/min.

### 8,9-Difluoro-2H-thiopyrano[3,4-c]isoquinoline-1,6(4H,5H)-dione (IVh)

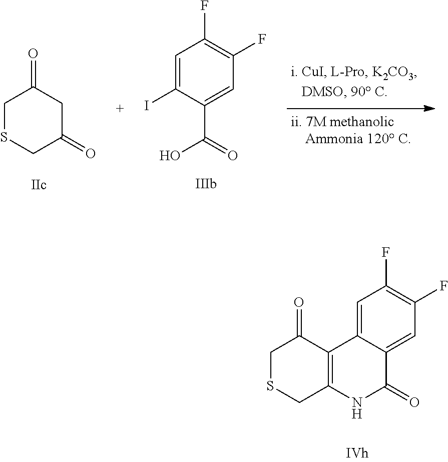 [SMILES: O=C1CSCC(=O)C1 ; O=C(O)c1cc(F)c(F)cc1I ; O=C1CSCc2[nH]c(=O)c3cc(F)c(F)cc3c21]

Step i: A mixture of 5.0 g (17.6 mmol, 1.0 eq.) of 4,5-difluoro-2-iodobenzoic acid (IIIb), 2.74 g (21.12 mmol, 1.2 eq.) of 2H-thiopyran-3,5(4H,6H)-dione (IIc), 9.7 g (70.4 mmol, 4.0 eq.) of potassium carbonate, 0.41 g (3.5 mmol, 0.2 eq.) of L-proline and 0.33 g (1.17 mmol, 0.1 eq.) of copper(I)iodide in 30 mL of dry DMSO under a nitrogen atmosphere was stirred at 110° C. for 16 h (Note: Reaction was performed on 4×5 g scale in parallel). On cooling to room temperature, the reaction mixtures were combined and diluted with cold water (100 mL) and acidified with 2 M HCl solution (30 mL). The resulting suspension was filtered, and the filtrate was extracted with ethyl acetate (3×500 mL). The combined organic extracts were washed with brine (150 mL), dried over anhydrous Na₂SO₄, filtered and concentrated under reduced pressure to afford 15.2 g of 8,9-difluorothiopyrano[3,4-c]isochromene-1,6(2H,4H)-dione and 4,5-difluoro-2-(5-hydroxy-3-oxo-3,6-dihydro-2H-thiopyran-4-yl)benzoic acid which was taken as such for next step without purification.

Step ii: To a mixture of 5 g (1.86 mmol, 1.0 eq.) of above prepared crude mixture of 8,9-difluorothiopyrano[3,4-c]isochromene-1,6(2H,4H)-dione and 4,5-difluoro-2-(5-hydroxy-3-oxo-3,6-dihydro-2H-thiopyran-4-yl)benzoic acid in a steel bomb at −35° C. was added 100 mL of 7 M methanolic ammonia. The vessel was sealed and the mixture was heated at 120° C. for 1 h. The mixture was then allowed to cool to room temperature and concentrated under reduced pressure. The residue was stirred with 10 vol of DMSO:Water (1:9) for 30 min to obtain a solid which was filtered and washed with water to afford 1.3 g (4.8 mmol, 26%) of 8,9-difluoro-2H-thiopyrano[3,4-c]isoquinoline-1,6(4H,5H)-dione (IVh). LCMS: m/z found 266.17 [M−H]⁻.

### 8,9-Difluoro-1-(methylamino)-1,5-dihydro-2H-thiopyrano[3,4-c]isoquinolin-6(4H)-one (Vk)

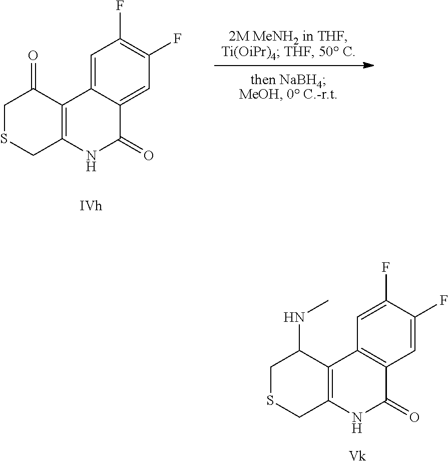 [SMILES: O=C1CSCc2[nH]c(=O)c3cc(F)c(F)cc3c21 ; "2M MeNH2 in THF,Ti(OiPr)4; THF, 50° C." ; "then NaBH4;MeOH, 0° C.-r.t." ; CNC1CSCc2[nH]c(=O)c3cc(F)c(F)cc3c21]

Racemic 8,9-difluoro-1-(methylamino)-1,5-dihydro-2H-thiopyrano[3,4-c]isoquinolin-6(4H)-one was synthesized in an analogous manner as described above, from 8,9-difluoro-2H-thiopyrano[3,4-c]isoquinoline-1,6(4H,5H)-dione (IVh), and methylamine. LCMS: m/z found 283.3 [M+H]⁺.

### N-(8,9-Difluoro-6-oxo-1,4,5,6-tetrahydro-2H-thiopyrano[3,4-c]isoquinolin-1-yl)-8-fluoro-N-methylindolizine-2-carboxamide (Compound 103 and 104)

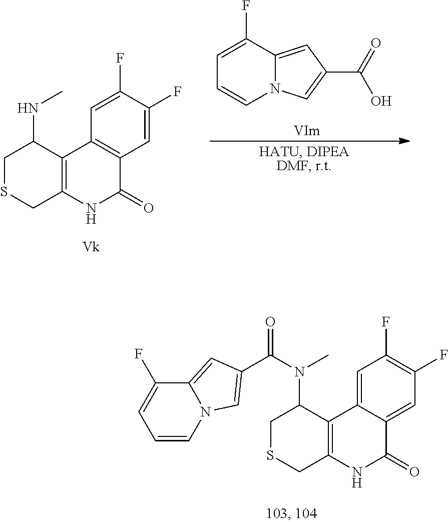

Racemic N-(8,9-difluoro-6-oxo-1,4,5,6-tetrahydro-2H-thiopyrano[3,4-c]isoquinolin-1-yl)-8-fluoro-N-methylindolizine-2-carboxamide was synthesized in an analogous manner as described above, from racemic 8,9-difluoro-1-(methylamino)-1,5-dihydro-2H-thiopyrano[3,4-c]isoquinolin-6(4H)-one (Vk) and 8-fluoroindolizine-2-carboxylic acid (VIm). The enantiomers were subsequently separated by preparative SFC: Method isocratic, Mobile phase Methanol:CO₂—40:60. Column: Chiralpak-IC (30×250 mm), 5 μm, flow rate: 110 g/min.

Enantiomer I (Compound 103): LCMS: m/z found 444.2 [M+H]⁺, RT=5.51 min, (Method A); ¹H NMR (400 MHz, DMSO-d₆) δ 11.71 (br s, 1H), 8.15-8.08 (m, 3H) 7.27-7.22 (m, 1H), 6.76 (s 1H), 6.67-6.62 (m, 2H), 5.89 (s, 1H), 3.79 (d, 1H), 3.67 (d, 1H), 3.49-3.41 (m, 1H), 3.11-3.01 (m, 1H), 3.02 (s, 3H); Chiral analytical SFC: RT=5.14 min, Column: Chiralpak IC-3 (4.6×150 mm) 3 μm, 40% of MeOH, Flow rate: 3.0 g/min.

Enantiomer II (Compound 104): LCMS: m/z found 444.2 [M+H]⁺, RT=5.51 min, (Method A); ¹H NMR (400 MHz, DMSO-d₆) δ 11.71 (br s, 1H), 8.15-8.08 (m, 3H) 7.27-7.22 (m, 1H), 6.76 (s 1H), 6.67-6.62 (m, 2H), 5.89 (s, 1H), 3.79 (d, 1H), 3.67 (d, 1H), 3.49-3.41 (m, 1H), 3.11-3.01 (m, 1H), 3.02 (s, 3H); Chiral analytical SFC: RT=6.60 min, Column: Chiralpak IC-3 (4.6×150 mm) 3 μm, 40% of MeOH, Flow rate: 3.0 g/min.

### 8,9-Difluoro-1-(methylamino)-1,5-dihydro-2H-thiopyrano[3,4-c]isoquinolin-6(4H)-one 3-oxide (Vp)

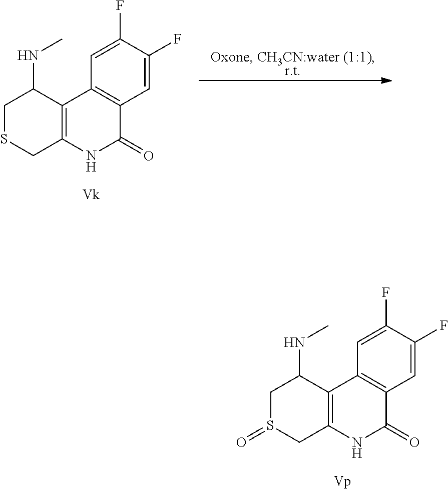 [SMILES: CNC1CSCc2[nH]c(=O)c3cc(F)c(F)cc3c21 ; "Oxone, CH3CN:water (1:1),r.t." ; CNC1CS(=O)Cc2[nH]c(=O)c3cc(F)c(F)cc3c21]

To a stirred solution of 850 mg (282 mmol, 1 eq.) of 8,9-difluoro-1-(methylamino)-1,5-dihydro-2H-thiopyrano[3,4-c]isoquinolin-6(4H)-one (Vk) in 10 mL of acetonitrile:water (1:1) at room temperature, 740 mg (2.44 mmol, 0.8 eq.) of oxone was added and the resulting reaction mixture was stirred for 6 h. The mixture was then concentrated and diluted with methanol (20 mL). After filtering the suspension, the filtrate was concentrated under reduced pressure to afford crude (700 mg) 8,9-difluoro-1-(methylamino)-1,5-dihydro-2H-thiopyrano[3,4-c]isoquinolin-6(4H)-one 3-oxide (Vp). This material was used without further purification in the next steps. LCMS: m/z found 299.24 [M+H]⁺.

### N-(8,9-Difluoro-3-oxido-6-oxo-1,4,5,6-tetrahydro-2H-thiopyrano[3,4-c]isoquinolin-1-yl)-8-fluoro-N-methylindolizine-2-carboxamide (Compound 149 and 150)

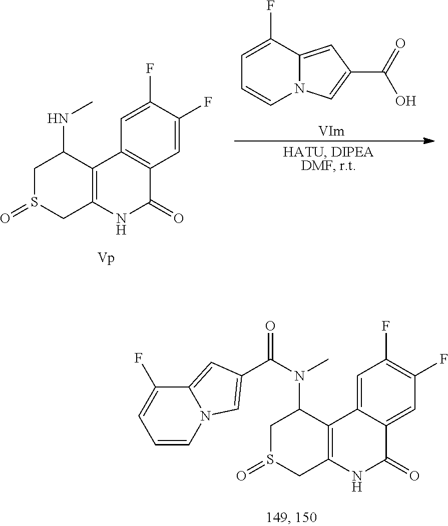

N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide (mixture of four stereoisomers) was synthesized in an analogous manner as described above, from racemic 8,9-difluoro-1-(methylamino)-1,5-dihydro-2H-thiopyrano[3,4-c]isoquinolin-6(4H)-one 3-oxide (Vp) and 8-fluoroindolizine-2-carboxylic acid (VIm). The racemic diastereoisomers were subsequently separated by preparative SFC: Method isocratic, Mobile phase Methanol:CO₂—35:65. Column: DCPAK P4VP (20×250 mm) 5 μm, flow rate: 60 g/min.

Diastereoisomer I, racemic (Compound 150): LCMS: m/z found 460.3. [M+H]⁺, RT=3.45 min, (Method A); ¹H NMR (400 MHz, DMSO-d₆) δ 11.72 (s, 1H), 8.18-8.10 (m, 3H), 7.34-7.29 (m, 1H), 6.77 (s, 1H), 6.65-6.63 (m, 2H), 6.29 (t, 1H), 4.38 (d, 1H), 3.90 (d, 1H), 3.62-3.58 (m, 1H), 3.20-3.15 (m, 1H), 2.81 (s, 3H); Chiral analytical SFC: RT=6.02 and 10.66 min, Column: Chiralcel OX-3 (4.6×150 mm) 3 μm, 50% (0.5% DEA in Methanol), Flow rate: 3.0 g/min.

Diastereoisomer II, racemic (Compound 149): LCMS: m/z found 460.3 [M+H]⁺, RT=3.54 min, (Method A); ¹H NMR (400 MHz, DMSO-d₆) δ 11.71 (s, 1H), 8.16-8.08 (m, 3H), 7.32-7.27 (m, 1H), 6.75 (s, 1H), 6.64-6.63 (m, 2H), 6.25 (t, 1H), 4.18-4.13 (m, 2H), 3.68-3.64 (m, 1H), 3.32 (m, 1H), 2.87 (s, 3H); Chiral analytical SFC: RT=7.52 and 9.86 min, Column: Chiralcel OX-3 (4.6×150 mm) 3 μm, 50% (0.5% DEA in Methanol), Flow rate: 3.0 g/min.

### 8,9-Difluoro-1-(methylamino)-1,5-dihydro-2H-thiopyrano[3,4-c]isoquinolin-6(4H)-one 3,3-dioxide (Vq)

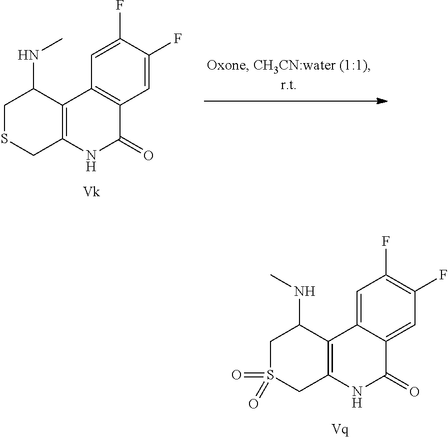 [SMILES: CNC1CSCc2[nH]c(=O)c3cc(F)c(F)cc3c21 ; CNC1CS(=O)(=O)Cc2[nH]c(=O)c3cc(F)c(F)cc3c21]

To a stirred solution of 700 mg (2.48 mmol, 1 eq.) of 8,9-difluoro-1-(methylamino)-1,5-dihydro-2H-thiopyrano[3,4-c]isoquinolin-6(4H)-one (Vk) in 5 mL of ACN and 5 mL of water at 0° C., 2.28 g (7.44 mmol, 3 eq.) of oxone was added and the resulting reaction mixture was stirred at room temperature for 16 h. After completion of reaction, the reaction mixture was concentrated under reduced pressure to afford a residue which was diluted with methanol (20 mL) and stirred for 20 min. After filtering the suspension, the filtrate was concentrated under reduced pressure to give a crude product, which was triturated with diethyl ether (10 mL) to afford 700 mg (2.22 mmol, 89% yield) of 8,9-difluoro-1-(methylamino)-1,5-dihydro-2H-thiopyrano[3,4-c]isoquinolin-6(4H)-one 3,3-dioxide (Vq) as a brown solid. LCMS m/z found 315.24 [M+H]⁺, RT=0.50 min (Method E).

### N-(8,9-Difluoro-3,3-dioxido-6-oxo-2,4,5,6-tetrahydro-1H-thiopyrano[3,4-c]isoquinolin-1-yl)-8-fluoro-N-methylindolizine-2-carboxamide (Compound 151 and 152)

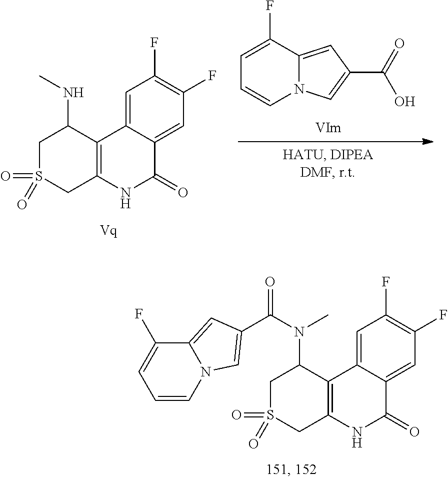

Racemic N-(8,9-difluoro-3,3-dioxido-6-oxo-2,4,5,6-tetrahydro-1H-thiopyrano[3,4-c]isoquinolin-1-yl)-8-fluoro-N-methylindolizine-2-carboxamide was synthesized in an analogous manner as described above, from racemic 8,9-difluoro-1-(methylamino)-1,5-dihydro-2H-thiopyrano[3,4-c]isoquinolin-6(4H)-one 3,3-dioxide (Vq) and 8-fluoroindolizine-2-carboxylic acid (VIm). The enantiomers were subsequently separated by preparative SFC: Method isocratic, Mobile phase Methanol:CO₂—50:50. Column: Chiralpak-OD-H (30×250 mm), 5 μm, flow rate: 110 g/min.

Enantiomer I (Compound 151): LCMS: m/z found 476.2 [M+H]⁺, RT=3.84 min, (Method A); ¹H NMR (400 MHz, DMSO-d₆) δ 11.73 (s, 1H), 8.16-8.10 (m, 3H), 7.29 (m, 1H), 6.77 (s, 1H), 6.65-6.63 (m, 2H), 6.26 (t, 1H), 4.85 (d, 1H), 4.13 (d, 1H), 3.80 (m, 1H), 3.70-3.64 (m, 1H), 2.84 (s, 3H); Chiral analytical SFC: RT=2.29 min, Column: Chiralcel OD-3 (4.6×150 mm) 3 μm, 50% of MeOH, Flow rate: 3.0 g/min.

Enantiomer II (Compound 152): LCMS: m/z found 476.2 [M+H]⁺, RT=3.84 min, (Method A); ¹H NMR (400 MHz, DMSO-d₆) δ 11.73 (s, 1H), 8.16-8.10 (m, 3H), 7.29 (m, 1H), 6.77 (s, 1H), 6.65-6.63 (m, 2H), 6.26 (t, 1H), 4.85 (d, 1H), 4.13 (d, 1H), 3.80 (m, 1H), 3.70-3.64 (m, 1H), 2.84 (s, 3H); Chiral analytical SFC: RT=4.64 min, Column: Chiralcel OD-3 (4.6×150 mm) 3 μm, 50% of MeOH, Flow rate: 3.0 g/min.

### 4,5-Dihydropyrano[3,4-c]isoquinoline-1,6-dione (IVi)

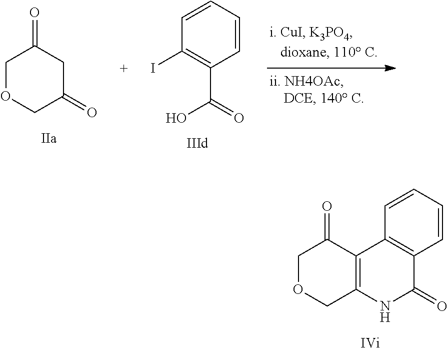 [SMILES: O=C1COCC(=O)C1 ; O=C(O)c1ccccc1I ; O=C1COCc2[nH]c(=O)c3ccccc3c21]

Step i: 2-Iodobenzoic acid (IIId, 0.99 g, 4.0 mmol), tetrahydropyran-3,5-dione, (IIa, 0.55 g, 4.8 mmol), copper (I) iodide (76 mg, 0.4 mmol), and potassium phosphate (1.19 g, 5.6 mmol) were combined in a tube under a nitrogen atmosphere. Anhydrous 1,4-dioxane (10 mL) was added and the reaction tube was purged with nitrogen and stirred at room temperature for 30 min, and then at 110° C. for a further 3 h. The reaction mixture was diluted with ethyl acetate (10 mL), filtered through CELITE® and the pad was washed with ethyl acetate (3×10 mL). The filtrate was evaporated under high vacuum and the residue was purified by flash chromatography (silica gel, ethyl acetate/hexanes 0-90%) to provide 0.41 g (47% yield) of 4H-pyrano[3,4-c]isochromene-1,6-dione. LCMS m/z found 217.1 [M+H]⁺; RT=0.94 min (Method B); ¹H NMR (400 MHz, CDCl₃) δ 8.92 (m, 1H), 8.30 (m, 1H), 7.84 (m, 1H), 7.59 (m, 1H), 4.74 (d, 2H), 4.36-4.30 (m, 2H).

Step ii: 4H-Pyrano[3,4-c]isochromene-1,6-dione (80 mg, 0.37 mmol) and ammonium acetate (0.17 g, 2.22 mmol) were stirred in 1,2-dichloroethane (4 mL) at 140° C. in a sealed tube for 7 h. The reaction mixture was cooled to room temperature, diluted with dichloromethane/methanol, and adsorbed onto silica gel. The product was isolated by flash chromatography (silica gel, dryloaded, MeOH/DCM 0-4%) to afford 60 mg (75% yield) of 4,5-dihydropyrano[3,4-c]isoquinoline-1,6-dione. LCMS: m/z found 216.1 [M+H]⁺; RT=0.87 min (Method B); ¹H NMR (400 MHz, DMSO-d₆) δ 12.12 (s, 1H), 9.02 (m, 1H), 8.23 (m, 1H), 7.82 (m, 1H), 7.56 (m, 1H), 4.79 (d, 2H), 4.27 (d, 2H).

### 1-(Methylamino)-1,2,4,5-tetrahydropyrano[3,4-c]isoquinolin-6-one (VI)

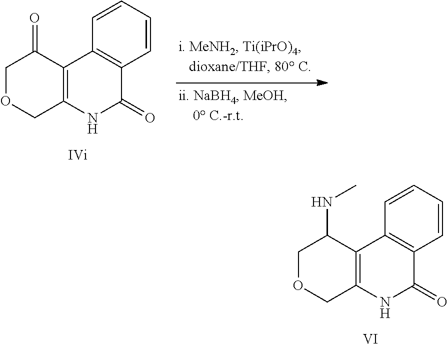

Tetraisopropoxytitanium (0.56 mL, 1.86 mmol) was added to a mixture of 4,5-dihydropyrano[3,4-c]isoquinoline-1,6-dione (IVi, 0.10 g, 0.46 mmol) and a 2 M methylamine solution in THF (0.46 mL, 0.93 mmol), and 1,4-dioxane (5 mL). The mixture was then stirred under nitrogen at 80° C. for 3 h. The reaction mixture was diluted with 2 mL of anhydrous MeOH, cooled to 0° C., treated with sodium borohydride (35 mg, 0.93 mmol) and allowed to stir for 1 h. The reaction was quenched by addition of brine (1.5 mL), diluted with 20 mL of ethyl acetate, and stirred for additional 15 min. The mixture was filtered through CELITE®, and the filter cake was washed with an additional 25 mL ethyl acetate. The combined filtrate was dried over sodium sulfate, filtered, and the solvent was evaporated under reduced pressure. The product was isolated by flash-chromatography (silica gel, MeOH/DCM 0-10%) to afford 86 mg (80% yield) of racemic 1-(methylamino)-1,2,4,5-tetrahydropyrano[3,4-c]isoquinolin-6-one. LCMS: m/z found 200.1 [M−(MeNH)]⁺; RT=0.59 min, (Method B); ¹H NMR (400 MHz, CDCl₃) δ 11.61 (s, 1H), 8.46-8.39 (m, 1H), 7.79-7.68 (m, 2H), 7.49 (ddd, 1H), 4.72 (d, 1H), 4.60 (dd, 1H), 4.42 (d, 1H), 3.69-3.60 (m, 2H), 2.62 (s, 3H).

### 3-Fluoro-N-methyl-N-(6-oxo-1,4,5,6-tetrahydro-2H-pyrano[3,4-c]isoquinolin-1-yl)-4-(trifluoromethyl)benzamide (Compound 132)

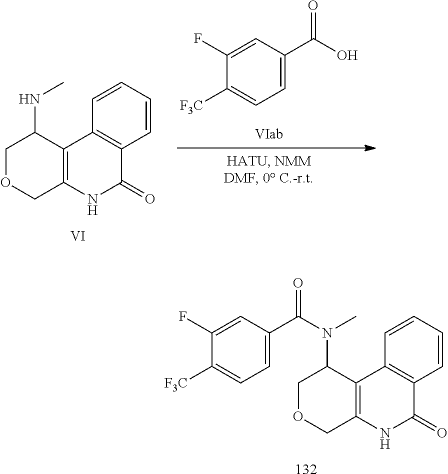

Racemic 3-fluoro-N-methyl-N-(6-oxo-1,4,5,6-tetrahydro-2H-pyrano[3,4-c]isoquinolin-1-yl)-4-(trifluoromethyl)benzamide was synthesized in an analogous manner as described above from racemic 1-(methylamino)-1,2,4,5-tetrahydropyrano[3,4-c]isoquinolin-6-one (VI), and 3-fluoro-4-(trifluoromethyl)benzoic acid (VIab). LCMS m/z found 421.1 [M+H]⁺; RT=5.67 min (Method A); ¹H NMR (400 MHz, DMSO-d₆) δ 11.49 (s, 1H), 8.28-8.18 (m, 1H), 7.97-7.79 (m, 2H), 7.64 (d, 1H), 7.61-7.49 (m, 2H), 7.45-7.38 (m, 1H), 5.68 (t, 1H), 4.59 (d, 1H), 4.47 (dd, 1H), 4.29 (d, 1H), 4.03 (dd, 1H), 2.66 (s, 3H).

### 6H-Pyrano[3,4-b]thieno[3,4-d]pyridine-4,9(5H,8H)-dione (IVj)

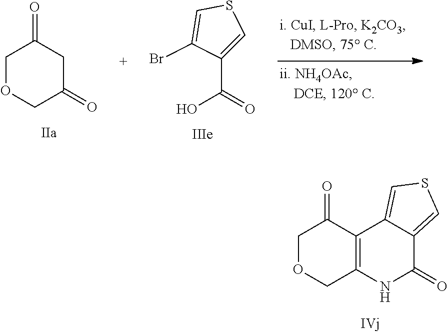 [SMILES: O=C1COCC(=O)C1 ; O=C(O)c1cscc1Br ; O=C1COCc2[nH]c(=O)c3cscc3c21]

6H-Pyrano[3,4-b]thieno[3,4-d]pyridine-4,9(5H,8H)-dione was synthesized in an analogous manner as described above, from 4-bromothiophene-3-carboxylic acid (IIIe) and tetrahydropyran-3,5-dione (IIa). LCMS m/z found 222.1 [M+H]⁺; RT=0.58 min (Method B); ¹H NMR (400 MHz, DMSO-d₆) δ 12.28 (s, 1H), 7.71 (d, 1H), 7.51 (d, 1H), 4.83 (s, 2H), 4.35 (s, 2H).

### 9-(Methylamino)-8,9-dihydro-6H-pyrano[3,4-b]thieno[3,4-d]pyridin-4(5H)-one (Vm)

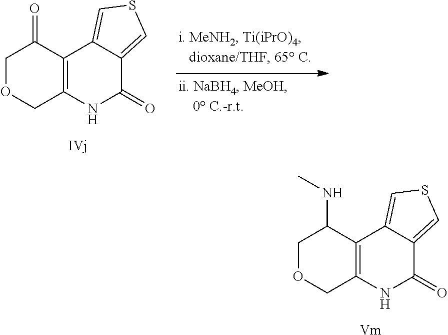

9-(Methylamino)-8,9-dihydro-6H-pyrano[3,4-b]thieno[3,4-d]pyridin-4(5H)-one was synthesized in an analogous manner as described above, from 6H-pyrano[3,4-b]thieno[3,4-d]pyridine-4,9(5H,8H)-dione (IVj) and methylamine. LCMS m/z found 237.1 [M+H]⁺; RT=0.40 min (Method B); ¹H NMR (400 MHz, CDCl₃) δ 10.72 (s, 1H), 8.37 (dd, 1H), 7.45-7.38 (m, 1H), 4.58 (d, 1H), 4.47 (dd, 1H), 4.29 (dd, 1H), 3.64 (dd, 1H), 3.52 (p, 1H), 2.58 (s, 3H).

### 3-Fluoro-N-methyl-N-(4-oxo-4,5,8,9-tetrahydro-6H-pyrano[3,4-b]thieno[3,4-d]pyridin-9-yl)-4-(trifluoromethyl)benzamide (Compound 133)

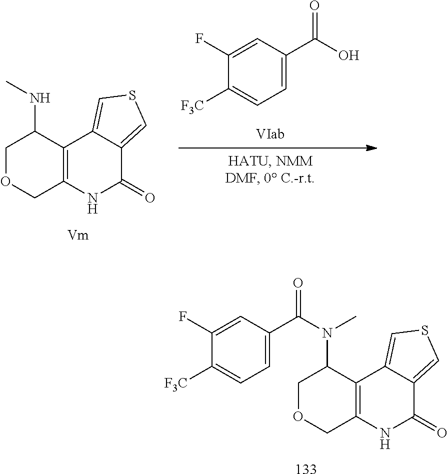

Racemic 3-fluoro-N-methyl-N-(4-oxo-4,5,8,9-tetrahydro-6H-pyrano[3,4-b]thieno[3,4-d]pyridin-9-yl)-4-(trifluoromethyl)benzamide was synthesized in an analogous manner as described above from racemic 9-(methylamino)-8,9-dihydro-6H-pyrano[3,4-b]thieno[3,4-d]pyridin-4(5H)-one (Vm), and 3-fluoro-4-(trifluoromethyl)benzoic acid (VIab). LCMS m/z found 427.1 [M+H]⁺; RT=5.60 min (Method A); ¹H NMR (400 MHz, DMSO-d₆) δ 10.83 (s, 1H), 8.53-8.44 (m, 1H), 7.88 (t, 1H), 7.72 (d, 1H), 7.55-7.47 (m, 2H), 5.60 (d, 1H), 4.48 (d, 1H), 4.40-4.20 (m, 2H), 4.00 (dd, 1H), 2.69 (s, 3H).

### 1,6-Dioxo-4,5-dihydropyrano[3,4-c]isoquinoline-8-carbonitrile (IVk)

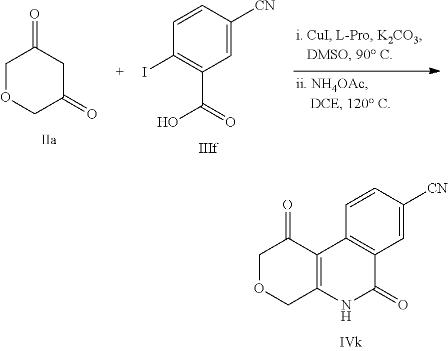 [SMILES: O=C1COCC(=O)C1 ; N#Cc1ccc(I)c(C(=O)O)c1 ; N#Cc1ccc2c3c([nH]c(=O)c2c1)COCC3=O]

1,6-Dioxo-4,5-dihydropyrano[3,4-c]isoquinoline-8-carbonitrile was synthesized in an analogous manner as described above, from 5-cyano-2-iodo-benzoic acid (IIIf) and tetrahydropyran-3,5-dione (IIa). LCMS m/z found 241.2 [M+H]⁺, RT=2.17 min (Method C); ¹H NMR (400 MHz, DMSO-d₆) δ 12.45 (s, 1H), 9.11 (d, 1H), 8.53 (d, 1H), 8.16 (dd, 1H), 4.80 (s, 2H), 4.29 (s, 2H).

### 1-(Methylamino)-6-oxo-1,2,4,5-tetrahydropyrano[3,4-c]isoquinoline-8-carbonitrile (Vn)

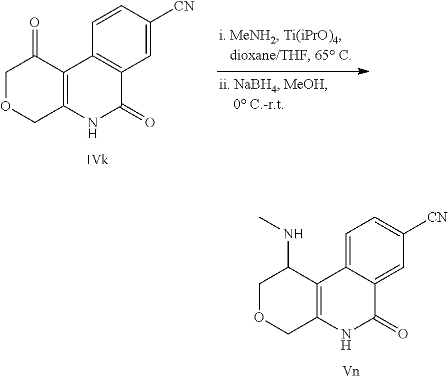 [SMILES: N#Cc1ccc2c3c([nH]c(=O)c2c1)COCC3=O ; CNC1COCc2[nH]c(=O)c3cc(C#N)ccc3c21]

1-(Methylamino)-6-oxo-1,2,4,5-tetrahydropyrano[3,4-c]isoquinoline-8-carbonitrile was synthesized in an analogous manner as described above, from 1,6-dioxo-4,5-dihydropyrano[3,4-c]isoquinoline-8-carbonitrile (IVk) and methanamine. LCMS m/z found 256.2 [M+H]⁺, RT=0.44 min (Method B); ¹H NMR (400 MHz, Methanol-d₄) δ 8.63-8.58 (m, 1H), 7.93 (dd, 1H), 7.84 (d, 1H), 4.59 (d, 1H), 4.48 (dd, 1H), 4.42-4.33 (m, 1H), 3.70-3.55 (m, 2H), 2.55 (s, 3H).

### N-(8-Cyano-6-oxo-1,4,5,6-tetrahydro-2H-pyrano[3,4-c]isoquinolin-1-yl)-3-fluoro-N-methyl-4-(trifluoromethyl)benzamide (Compound 134)

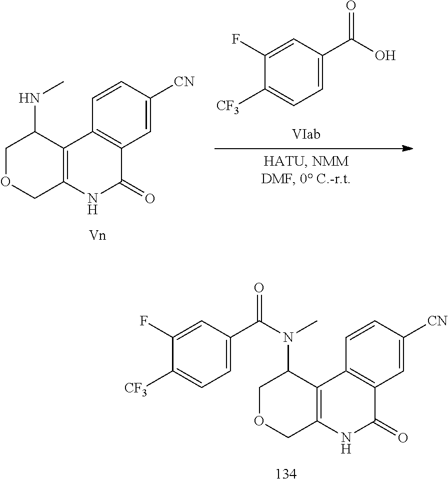

Racemic N-(8-cyano-6-oxo-1,4,5,6-tetrahydro-2H-pyrano[3,4-c]isoquinolin-1-yl)-3-fluoro-N-methyl-4-(trifluoromethyl)benzamide was synthesized in an analogous manner as described above from racemic 1-(methylamino)-6-oxo-1,2,4,5-tetrahydropyrano[3,4-c]isoquinoline-8-carbonitrile (Vn), and 3-fluoro-4-(trifluoromethyl)benzoic acid (VIab). LCMS m/z found 446.2 [M+H]⁺; RT=5.72 min (Method A); ¹H NMR (400 MHz, DMSO-d₆) δ 11.88 (s, 1H), 8.57 (d, 1H), 8.20 (dd, 1H), 7.88 (t, 1H), 7.67 (d, 2H), 7.45 (d, 1H), 5.74-5.68 (m, 1H), 4.63 (d, 1H), 4.50 (dd, 1H), 4.31 (d, 1H), 4.04 (dd, 1H), 2.66 (s, 3H).

### (S)-3-Chloro-N-(8,9-difluoro-6-oxo-1,4,5,6-tetrahydro-2H-pyrano[3,4-c]isoquinolin-1-yl)-4-fluoro-N-methylbenzenesulfonamide (Compound 143)

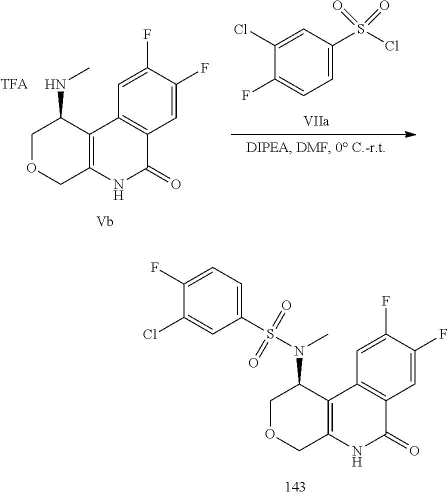

A stirred solution of DIPEA (105 μL, 0.60 mmol) and enantiomerically pure (S)-8,9-difluoro-1-(methylamino)-1,5-dihydro-2H-pyrano[3,4-c]isoquinolin-6(4H)-one (Vb, 56 mg TFA salt, 0.15 mmol) in DMF (1 mL) was treated dropwise with a solution of 3-chloro-4-fluoro-benzenesulfonyl chloride (VIIa, 23.5 uL, 0.17 mmol) in 200 μL of DMF at 0° C. stirred for 10 min, then warmed to room temperature and stirred for an additional 1 h. The reaction was quenched by addition of 2.5 mL of saturated ammonium chloride and 2.5 mL of water, and the pH was adjusted to 3-4 (dropwise with 2 M HCl), and then extracted with ethyl acetate (2×30 mL). The combined organic extracts were washed saturated ammonium chloride (2×25 mL), followed by saturated sodium bicarbonate (2×25 mL), water (25 mL) and brine (25 mL), and then dried over sodium sulfate, filtered and the solvent evaporated. The product was isolated by flash chromatography (silica gel, methanol/dichloromethane 0-3% over 15 min) to afford enantiomerically pure (S)-3-chloro-N-(8,9-difluoro-6-oxo-1,4,5,6-tetrahydro-2H-pyrano[3,4-c]isoquinolin-1-yl)-4-fluoro-N-methylbenzenesulfonamide (12.9 mg, 19% yield): LCMS m/z found 459.1/461.1 [M+H]⁺; RT=4.88 min (Method A); ¹H NMR (400 MHz, Chloroform-d) δ 12.14 (s, 1H), 8.18 (dd, 1H), 8.00 (m, 1H), 7.91 (dd, 1H), 7.86-7.78 (m, 1H), 7.37 (t, 1H), 5.18-5.12 (m, 1H), 4.72 (d, 1H), 4.52 (dt, 1H), 3.91 (d, 1H), 3.76 (dd, 1H), 2.79 (s, 3H).

### (S)-3-Chloro-N-(8,9-difluoro-6-oxo-1,4,5,6-tetrahydro-2H-pyrano[3,4-c]isoquinolin-1-yl)-N-methylbenzenesulfonamide (Compound 144)

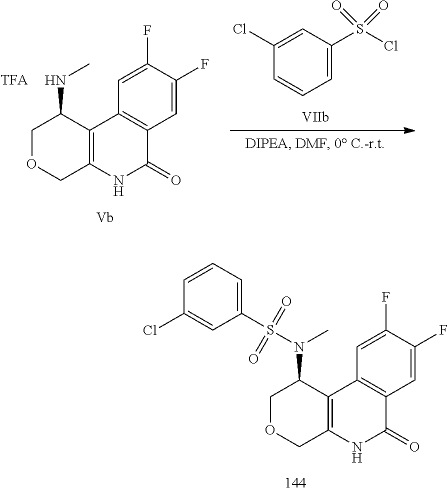

Enantiomerically pure (S)-3-chloro-N-(8,9-difluoro-6-oxo-1,4,5,6-tetrahydro-2H-pyrano[3,4-c]isoquinolin-1-yl)-N-methylbenzenesulfonamide was synthesized in an analogous manner as described above from enantiomerically pure (S)-8,9-difluoro-1-(methylamino)-1,5-dihydro-2H-pyrano[3,4-c]isoquinolin-6(4H)-one (Vb, TFA salt), and 3-chlorobenzenesulfonyl chloride (VIIb). LCMS m/z found 441.1/443.2 [M+H]⁺; RT=4.84 min (Method A); ¹H NMR (400 MHz, Chloroform-d) δ 12.06 (s, 1H), 8.17 (dd, 1H), 8.01-7.88 (m, 2H), 7.80 (ddd, 1H), 7.64 (ddd, 1H), 7.61-7.50 (m, 1H), 5.18-5.12 (m, 1H), 4.70 (d, 1H), 4.56-4.46 (m, 1H), 3.92-3.84 (m, 1H), 3.74 (dd, 1H), 2.79 (s, 3H).

### (S)-3-Cyano-N-(8,9-difluoro-6-oxo-1,4,5,6-tetrahydro-2H-pyrano[3,4-c]isoquinolin-1-yl)-N-methylbenzenesulfonamide (Compound 145)

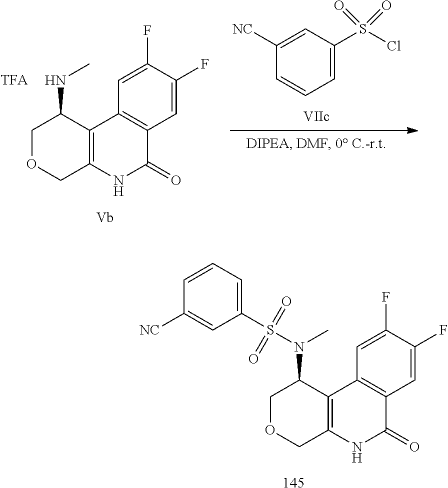

Enantiomerically pure (S)-3-cyano-N-(8,9-difluoro-6-oxo-1,4,5,6-tetrahydro-2H-pyrano[3,4-c]isoquinolin-1-yl)-N-methylbenzenesulfonamide was synthesized in an analogous manner as described above from enantiomerically pure (S)-8,9-difluoro-1-(methylamino)-1,5-dihydro-2H-pyrano[3,4-c]isoquinolin-6(4H)-one (Vb, TFA salt), and 3-cyanobenzenesulfonyl chloride (VIIc). LCMS m/z found 432.2 [M+H]⁺; RT=4.64 min (Method A); ¹H NMR (400 MHz, Chloroform-d) δ 11.70 (s, 1H), 8.24-8.11 (m, 3H), 7.95 (dt, 1H), 7.87 (dd, 1H), 7.76 (td, 1H), 5.17 (d, 1H), 4.69 (d, 1H), 4.52 (d, 1H), 3.90-3.81 (m, 1H), 3.76 (dd, 1H), 2.81 (s, 3H).

### (S)-3-Cyano-N-(8,9-difluoro-6-oxo-1,4,5,6-tetrahydro-2H-pyrano[3,4-c]isoquinolin-1-yl)-4-fluoro-N-methylbenzenesulfonamide (Compound 146)

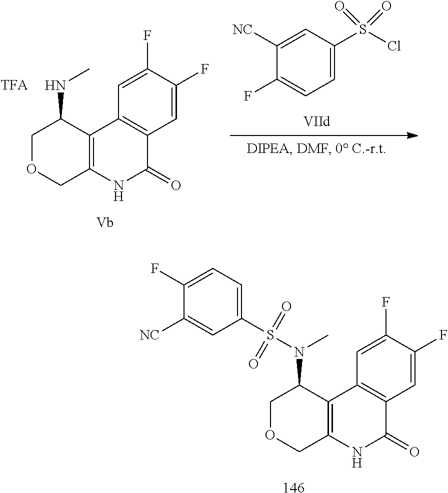

Enantiomerically pure (S)-3-cyano-N-(8,9-difluoro-6-oxo-1,4,5,6-tetrahydro-2H-pyrano[3,4-c]isoquinolin-1-yl)-4-fluoro-N-methylbenzenesulfonamide was synthesized in an analogous manner as described above from enantiomerically pure (S)-8,9-difluoro-1-(methylamino)-1,5-dihydro-2H-pyrano[3,4-c]isoquinolin-6(4H)-one (Vb, TFA salt), and 3-cyano-4-fluorobenzenesulfonyl chloride (VIId). LCMS m/z found 450.1 [M+H]⁺; RT=4.81 min (Method A); ¹H NMR (400 MHz, Chloroform-d) δ 11.60 (s, 1H), 8.26-8.11 (m, 3H), 7.86 (dd, 1H), 7.48 (dd, 1H), 5.18 (d, 1H), 4.69 (d, 1H), 4.57-4.48 (m, 1H), 3.89 (dd, 1H), 3.79 (dd, 1H), 2.80 (s, 3H).

### (S)-N-(8,9-Difluoro-5-methyl-6-oxo-1,4,5,6-tetrahydro-2H-pyrano[3,4-c]isoquinolin-1-yl)-5,6-difluoro-N-methyl-1H-indole-2-carboxamide (Compound 240)

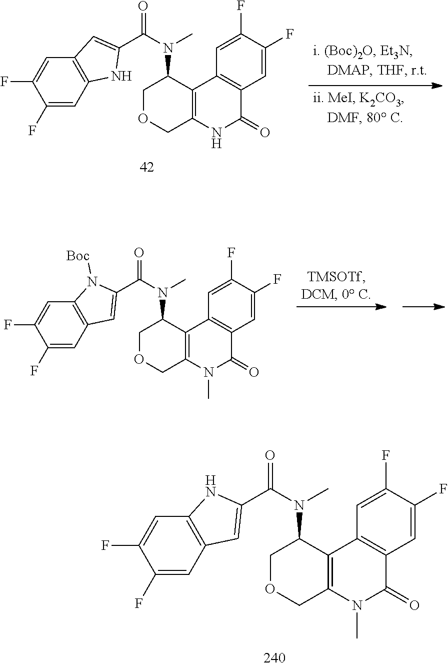 [SMILES: CN(C(=O)c1cc2cc(F)c(F)cc2[nH]1)[C@@H]1COCc2[nH]c(=O)c3cc(F)c(F)cc3c21 ; CN(C(=O)c1cc2cc(F)c(F)cc2n1C(=O)OC(C)(C)C)[C@@H]1COCc2c1c1cc(F)c(F)cc1c(=O)n2C ; CN(C(=O)c1cc2cc(F)c(F)cc2[nH]1)[C@@H]1COCc2c1c1cc(F)c(F)cc1c(=O)n2C]

Step i. To a stirred solution of 350 mg (0.785 mmol, 1 eq.) (S)-N-(8,9-difluoro-6-oxo-1,4,5,6-tetrahydro-2H-pyrano[3,4-c]isoquinolin-1-yl)-5,6-difluoro-N-methyl-1H-indole-2-carboxamide (42) in 3.5 mL of THF at room temperature were added 0.25 mL (1.8 mmol, 2.3 eq.) of Et₃N, 205 mg (0.94 mmol, 1.2 eq.) of di-tert-butyl dicarbonate and 9.5 mg (0.08 mmol, 0.1 eq.) of DMAP, and the reaction mixture was stirred at room temperature for 3 h. After completion of reaction (by TLC), the reaction mixture was quenched with water (20 mL) and extracted with EtOAc (2×70 mL). Combined organic layer was washed with water (20 mL), dried over Na₂SO₄ and concentrated under reduced pressure. Obtained material was purified by column chromatography using silica gel (100-200 mesh) and 80% EtOAc/petroleum ether as eluent to afford (250 mg, 58% yield) tert-butyl (S)-2-((8,9-difluoro-6-oxo-1,4,5,6-tetrahydro-2H-pyrano[3,4-c]isoquinolin-1-yl)(methyl)carbamoyl)-5,6-difluoro-1H-indole-1-carboxylate as pale brown solid.

Step ii. To a stirred solution of 80 mg (0.146 mmol, 1.0 eq.) of tert-butyl (S)-2-((8,9-difluoro-6-oxo-1,4,5,6-tetrahydro-2H-pyrano[3,4-c]isoquinolin-1-yl)(methyl)carbamoyl)-5,6-difluoro-1H-indole-1-carboxylate (from Step i) in 1 mL of DMF were added 20 mg (0.146 mmol, 1 eq.) of K₂CO₃ and 0.02 mL (0.293 mmol, 2 eq.) of methyl iodide at room temperature. The resulting mixture was stirred at 80° C. for 16 h. The reaction mixture was filtered through a pad of CELITE® and the CELITE® bed was washed with EtOAc (30 mL). Obtained filtrate was washed with water (20 mL), dried over anhydrous Na₂SO₄ and concentrated under reduced pressure to afford crude 70 mg (0.125 mmol, 85% yield) of tert-butyl (S)-2-((8,9-difluoro-5-methyl-6-oxo-1,4,5,6-tetrahydro-2H-pyrano[3,4-c]isoquinolin-1-yl)(methyl)carbamoyl)-5,6-difluoro-1H-indole-1-carboxylate.

Step iii. To the solution of 70 mg (0.125 mmol, 1 eq.) of tert-butyl (S)-2-((8,9-difluoro-5-methyl-6-oxo-1,4,5,6-tetrahydro-2H-pyrano[3,4-c]isoquinolin-1-yl)(methyl)carbamoyl)-5,6-difluoro-1H-indole-1-carboxylate (from Step ii) in DCM (0.5 mL) was added 0.045 mL (0.25 mmol, 2 eq.) of TMSOTf at 0° C. and the resulting reaction mixture was stirred for 1 h at the same temperature. After completion of the reaction (monitored by TLC), the volatiles were removed under reduced pressure. The resulting residue was taken in saturated NaHCO₃ solution (10 mL) and extracted with ethyl acetate (2×30 mL). Combined organic layer was washed with water (20 mL), dried over anhydrous Na₂SO₄ and concentrated under reduced pressure. Obtained crude material was purified by achiral SFC to afford 19 mg (33% yield) of (S)-N-(8,9-difluoro-5-methyl-6-oxo-1,4,5,6-tetrahydro-2H-pyrano[3,4-c]isoquinolin-1-yl)-5,6-difluoro-N-methyl-1H-indole-2-carboxamide, as an off-white solid. LCMS m/z found 460.3 [M+H]⁺; RT=7.03 min (Method A); ¹H NMR (400 MHz, DMSO-d₆) δ 11.92 (bs, 1H), 8.12 (t, 1H), 7.69 (t, 1H), 7.46-7.36 (m, 2H), 6.98 (s, 1H), 5.79 (s, 1H), 4.98 (d, 1H), 4.68 (d, 1H), 4.16 (d, 1H), 4.01 (d, 1H), 3.46 (s, 3H), 3.15 (s, 3H).

### (S)-N-(8,9-Difluoro-6-methoxy-1,4-dihydro-2H-pyrano[3,4-c]isoquinolin-1-yl)-5,6-difluoro-N-methyl-1H-indole-2-carboxamide (Compound 241)

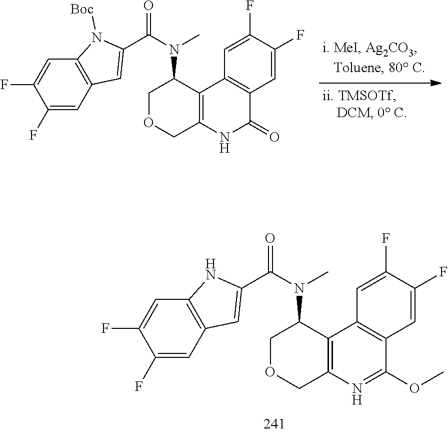 [SMILES: CN(C(=O)c1cc2cc(F)c(F)cc2n1C(=O)OC(C)(C)C)[C@@H]1COCc2[nH]c(=O)c3cc(F)c(F)cc3c21 ; COc1nc2c(c3cc(F)c(F)cc13)[C@H](N(C)C(=O)c1cc3cc(F)c(F)cc3[nH]1)COC2]

Step i. To a stirred solution of 80 mg (0.1467 mmol, 1 eq.) of tert-butyl (S)-2-((8,9-difluoro-6-oxo-1,4,5,6-tetrahydro-2H-pyrano[3,4-c]isoquinolin-1-yl)(methyl)carbamoyl)-5,6-difluoro-1H-indole-1-carboxylate (from Step i in the procedure above) in 1 mL of toluene were added 40 mg (0.1467 mmol, 1 eq.) of Ag₂CO₃ and 0.02 mL (0.29 mmol, 2 eq.) of methyl iodide at room temperature. The resulting mixture was stirred at 80° C. for 16 h. After completion of reaction (by TLC), the mixture was filtered through a pad of CELITE® and the CELITE® bed was washed with EtOAc (30 mL). Obtained filtrate was washed with water (20 mL), dried over anhydrous Na₂SO₄ and concentrated under reduced pressure to afford 80 mg of crude tert-butyl (S)-2-((8,9-difluoro-6-methoxy-1,4-dihydro-2H-pyrano[3,4-c]isoquinolin-1-yl)(methyl)carbamoyl)-5,6-difluoro-1H-indole-1-carboxylate as brown solid.

Step ii. Crude tert-butyl (S)-2-((8,9-difluoro-6-methoxy-1,4-dihydro-2H-pyrano[3,4-c]isoquinolin-1-yl)(methyl)carbamoyl)-5,6-difluoro-1H-indole-1-carboxylate obtained in Step i was converted to (S)-N-(8,9-difluoro-6-methoxy-1,4-dihydro-2H-pyrano[3,4-c]isoquinolin-1-yl)-5,6-difluoro-N-methyl-1H-indole-2-carboxamide by treatment with TMSOTf by a procedure similar to the one above, in Step iii. LCMS m/z found 460.3 [M+H]⁺; RT=7.91 min (Method A); ¹H NMR (400 MHz, DMSO-d₆) δ 11.94 (bs, 1H), 8.16 (t, 1H), 7.7-7.58 (m, 2H), 7.41-7.37 (m, 1H), 6.96 (s, 1H), 6.02 (s, 1H), 4.85 (d, 1H), 4.71 (d, 1H), 4.25 (d, 1H), 4.12 (m, 4H), 3.09 (s, 3H).

Benzyl (2-(((S)-8,9-difluoro-1-(((R)-1-(4-methoxyphenyl)ethyl)(methyl)amino)-1,4-dihydro-2H-pyrano[3,4-c]isoquinolin-6-yl)oxy)ethyl)carbamate (IXe) and benzyl (2-((S)-8,9-difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl)carbamate (IXd)

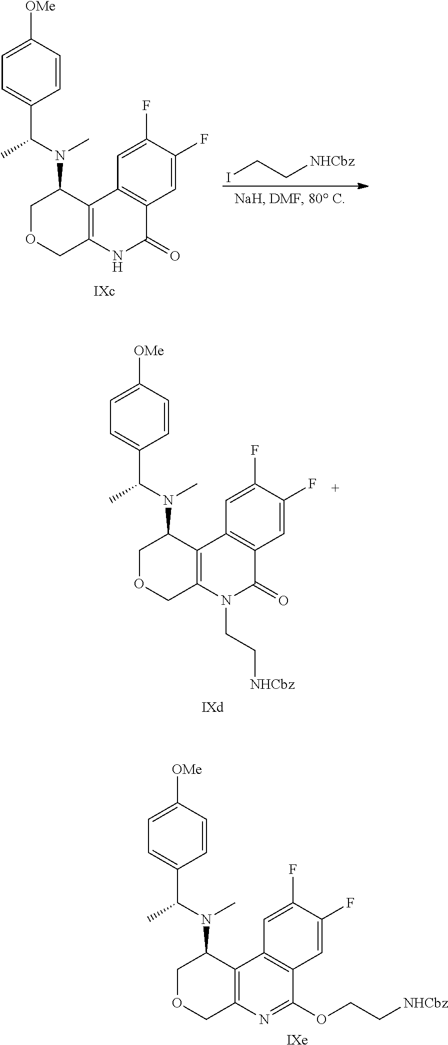

To a stirred solution of (500 mg, 0.12 mmol, 1.0 eq.) (S)-8,9-difluoro-1-(((R)-1-(4-methoxyphenyl)ethyl)(methyl)amino)-1,5-dihydro-2H-pyrano[3,4-c]isoquinolin-6(4H)-one (IXc) in 5 mL of DMF was added 36 mg of NaH (0.14 mmol, 1.2 eq.) at 0° C. and the reaction mixture was stirred for 15 min. 760 mg of benzyl (2-iodoethyl)carbamate (0.25 mmol, 2 eq.) were added and the reaction mixture was heated to 80° C. for 3 h. The mixture was cooled to room temperature, quenched with ice-water and extracted with EtOAc (2×100 mL). Combined organic layer was washed with ice-water, dried over anhydrous Na₂SO₄, filtered and the solvent was evaporated. Obtained material was purified by column chromatography (silica gel 100-200 mesh, 20% ethyl acetate in petroleum ether as a linear gradient) to afford 220 mg (42% yield) of benzyl (2-(((S)-8,9-difluoro-1-(((R)-1-(4-methoxyphenyl)ethyl)(methyl)amino)-1,4-dihydro-2H-pyrano[3,4-c]isoquinolin-6-yl)oxy)ethyl)carbamate (IXe) as a pale-yellow thick liquid. LCMS m/z found 578.70 [M+H]⁺, RT=1.77 min (Method E). Benzyl (2-((S)-8,9-difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl)carbamate (IXd) was also isolated (320 mg).

### Benzyl (S)-(2-((8,9-difluoro-1-(methylamino)-1,4-dihydro-2H-pyrano[3,4-c]isoquinolin-6-yl)oxy)ethyl)carbamate (Vr)

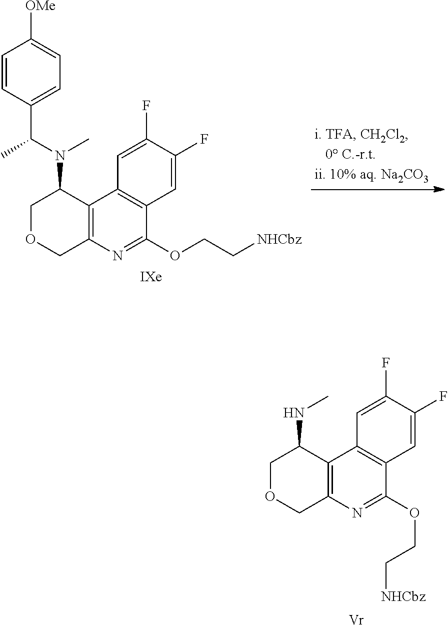

To a stirred solution of 400 mg (0.69 mmol, 1 eq.) benzyl (2-(((S)-8,9-difluoro-1-(((R)-1-(4-methoxyphenyl)ethyl)(methyl)amino)-1,4-dihydro-2H-pyrano[3,4-c]isoquinolin-6-yl)oxy)ethyl)carbamate (IXe) in 4 mL of DCM was added 2 mL of TFA at 0° C. and the reaction mixture was stirred for 12 h at room temperature. After completion of the reaction (monitored by TLC), the mixture was quenched with 10% aq. NaHCO₃ and extracted with EtOAc (2×50 mL). Combined organic layer was washed with ice-water, dried over anhydrous Na₂SO₄, filtered and the solvent was evaporated, to afford 20 mg of crude (benzyl (S)-(2-((8,9-difluoro-1-(methylamino)-1,4-dihydro-2H-pyrano[3,4-c]isoquinolin-6-yl)oxy)ethyl)carbamate (Vr), which was used in the next step without further purification. LCMS m/z found 444.32 [MH]⁺, RT=2.02 min (Method E).

### (S)-N-(6-(2-aminoethoxy)-8,9-difluoro-1,4-dihydro-2H-pyrano[3,4-c]isoquinolin-1-yl)-5,6-difluoro-N-methyl-1H-indole-2-carboxamide (Compound 280)

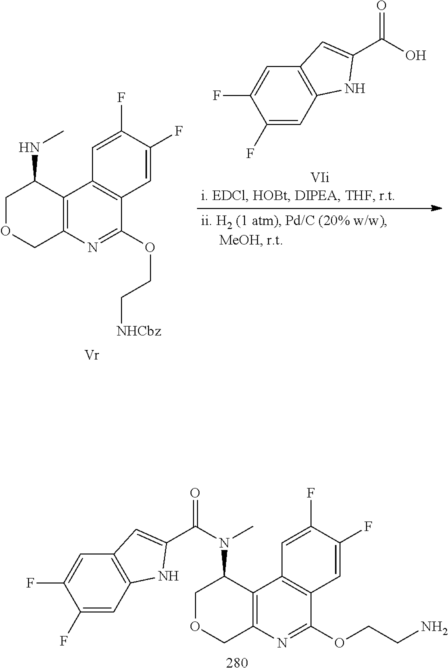

Step i. To a stirred solution of 160 mg (0.45 mmol, 1.2 eq.) of 5,6-difluoro-1H-indole-2-carboxylic acid (VIi) in 2 mL of DMF at room temperature were added 0.24 mL (1.35 mmol, 3 eq.) of DIPEA, 172 mg (1.12 mmol, 1.5 eq.) of HATU, followed by 80 mg (0.22 mmol, 1 eq.) of benzyl (S)-(2-((8,9-difluoro-1-(methylamino)-1,4-dihydro-2H-pyrano[3,4-c]isoquinolin-6-yl)oxy)ethyl)carbamate (Vr). The resulting mixture was stirred at room temperature for 16 h. After completion of reaction (by TLC), the reaction mixture was poured into ice cold water (20 mL) and stirred for 30 min. The solid formed from the reaction was collected by filtration, washed with Et₂O (2×10 mL) and dried under vacuum to afford 200 mg of crude benzyl (S)-(2-((1-(5,6-difluoro-N-methyl-1H-indole-2-carboxamido)-8,9-difluoro-1,4-dihydro-2H-pyrano[3,4-c]isoquinolin-6-yl)oxy)ethyl)carbamate, which was directly used in next step. LCMS m/z found 621.21 [M−H]⁻, RT=2.02 min (Method E).

Step ii. To a stirred solution of 200 mg (0.32 mmol, 1 eq.) of benzyl (S)-(2-((1-(5,6-difluoro-N-methyl-1H-indole-2-carboxamido)-8,9-difluoro-1,4-dihydro-2H-pyrano[3,4-c]isoquinolin-6-yl)oxy)ethyl)carbamate (from Step i) in 10 mL of MeOH was added 100 mg (0.11 mmol, 0.6 eq.) Pd/C and the mixture was stirred for 6 h under of hydrogen (1 atm). After completion of the reaction (monitored by TLC), the mixture was filtered through a pad of CELITE®, which was further washed with 10% DMF/MeOH (20 mL). Combined filtrate was concentrated under reduced pressure and the material was purified by preparative HPLC to afford 38 mg (32% yield) of (S)-N-(6-(2-aminoethoxy)-8,9-difluoro-1,4-dihydro-2H-pyrano[3,4-c]isoquinolin-1-yl)-5,6-difluoro-N-methyl-1H-indole-2-carboxamide. LCMS m/z found 489.2 [M+H]⁺; RT=6.71 min (Method A); ¹H NMR (400 MHz, DMSO-d₆) δ 11.93 (s, 1H), 8.35 (t, 1H), 7.68-7.58 (m, 2H), 7.41-7.37 (q, 1H), 6.96 (s, 1H), 6.01 (s, 1H), 4.82 (d, 1H), 4.68 (d, 1H), 4.43-4.38 (m, 2H), 4.26 (d, 1H), 4.11 (d, 1H), 3.09-2.99 (m, 7H).

### ((S)-N-(5-(2-Aminoethyl)-8,9-difluoro-6-oxo-1,4,5,6-tetrahydro-2H-pyrano[3,4-c]isoquinolin-1-yl)-5,6-difluoro-N-methyl-1H-indole-2-carboxamide (Compound 316)

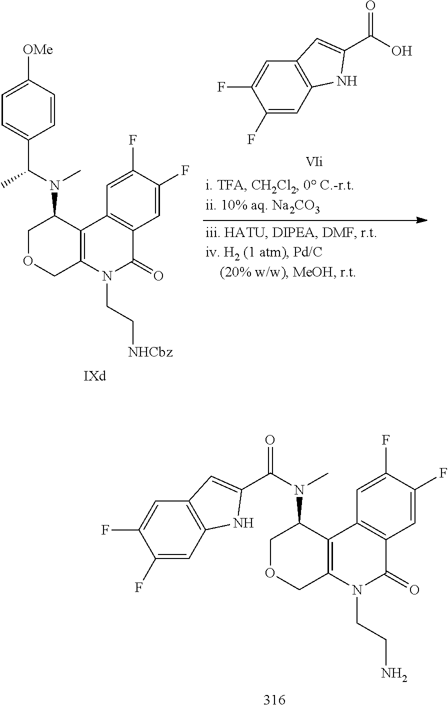 [SMILES: COc1ccc([C@@H](C)N(C)[C@@H]2COCc3c2c2cc(F)c(F)cc2c(=O)n3CCNC(=O)OCc2ccccc2)cc1 ; O=C(O)c1cc2cc(F)c(F)cc2[nH]1 ; CN(C(=O)c1cc2cc(F)c(F)cc2[nH]1)[C@@H]1COCc2c1c1cc(F)c(F)cc1c(=O)n2CCN]

Enantiomerically pure ((S)-N-(5-(2-aminoethyl)-8,9-difluoro-6-oxo-1,4,5,6-tetrahydro-2H-pyrano[3,4-c]isoquinolin-1-yl)-5,6-difluoro-N-methyl-1H-indole-2-carboxamide was synthesized in an analogous manner as described above from enantiomerically pure regioisomer benzyl (2-((S)-8,9-difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl)carbamate (IXd) and 5,6-difluoro-1H-indole-2-carboxylic acid (VIi). LCMS m/z found 489.2 [M+H]⁺; RT=3.56 min (Method A); ¹H NMR (400 MHz, DMSO-d₆) δ 11.92 (br s, 1H), 8.17 (m, 1H), 7.62 (m, 1H), 7.41 (m, 2H), 6.96 (s, 1H), 5.78 (s, 1H), 5.16 (d, 1H), 4.74 (d, 1H), 4.16 (d, 1H), 4.00-3.80 (m, 3H), 3.15 (s, 3H), 2.84 (t, 2H), 2.1 (br s, exch. Hs).

### 2-((S)-8,9-Difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl acetate (IXg) and 2-(((S)-8,9-difluoro-1-(((R)-1-(4-methoxyphenyl)ethyl)(methyl)amino)-1,4-dihydro-2H-pyrano[3,4-c]isoquinolin-6-yl)oxy)ethyl acetate (IXf)

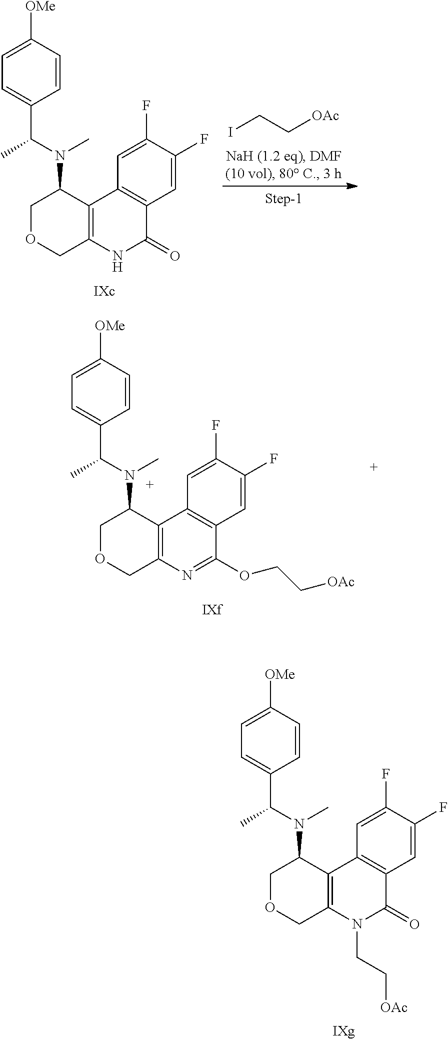

To a stirred solution of 600 mg (1.5 mmol, 1 eq.) 8,9-difluoro-1-(((R)-1-(4-methoxyphenyl)ethyl)(methyl)amino)-1,5-dihydro-2H-pyrano[3,4-c]isoquinolin-6(4H)-one (IXc) in mL of DMF was added 5 mg of NaH (2.2 mmol, 1.5 eq.) at 0° C. and the reaction mixture was stirred for 15 min. To this added 642 mg of 2-iodoethyl acetate (3.0 mmol, 2 eq.) and the reaction mixture was heated to 80° C. for 3 h. After completion of the reaction (monitored by TLC), the mixture was cooled to rt, quenched with ice-water and extracted with EtOAc (2×100 mL). Combined organic layer was washed with ice-water (50 mL), dried over anhydrous Na₂SO₄, filtered and the solvent was evaporated. Obtained material was purified by column chromatography (silicagel 100-200 mesh, 20% ethyl acetate in petroleum ether as a linear gradient) to afford 130 mg (26% yield) of 2-((S)-8,9-difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl acetate (IXg), LCMS m/z found 487.3 [M+H]⁺, RT=1.79 min, and 150 mg (20% yield) of 2-(((S)-8,9-difluoro-1-(((R)-1-(4-methoxyphenyl)ethyl)(methyl)amino)-1,4-dihydro-2H-pyrano[3,4-c]isoquinolin-6-yl)oxy)ethyl acetate (IXf), LCMS m/z found 487.3 [M+H]⁺, RT=1.87 min (Method E).

### (S)-2-(8,9-Difluoro-1-(methylamino)-6-oxo-1,2,4,6-tetrahydro-5H-pyrano[3,4-c]isoquinolin-5-yl)ethyl acetate (Vs)

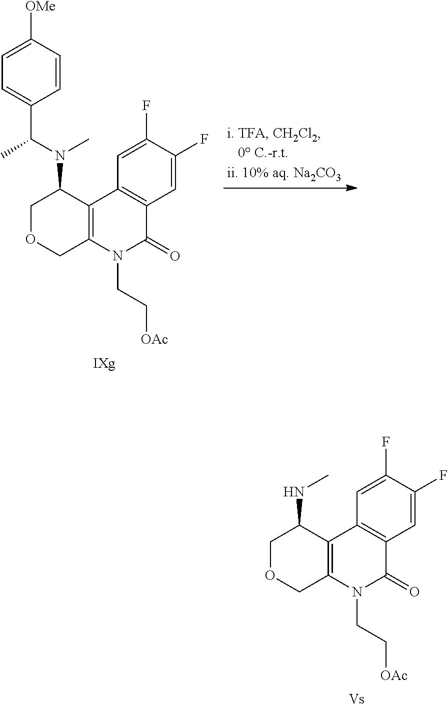 [SMILES: COc1ccc([C@@H](C)N(C)[C@@H]2COCc3c2c2cc(F)c(F)cc2c(=O)n3CCOC(C)=O)cc1 ; CN[C@@H]1COCc2c1c1cc(F)c(F)cc1c(=O)n2CCOC(C)=O]

Enantiomerically pure (S)-2-(8,9-difluoro-1-(methylamino)-6-oxo-1,2,4,6-tetrahydro-5H-pyrano[3,4-c]isoquinolin-5-yl)ethyl acetate (Vs) was synthesized in an analogous manner as described above, from 2-((S)-8,9-Difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl acetate (IXg). LCMS m/z found 352.9 [MH]⁺, RT=1.09 min (Method E).

### (S)-N-(8,9-Difluoro-5-(2-hydroxyethyl)-6-oxo-1,4,5,6-tetrahydro-2H-pyrano[3,4-c]isoquinolin-1-yl)-5,6-difluoro-N-methyl-1H-indole-2-carboxamide (Compound 315)

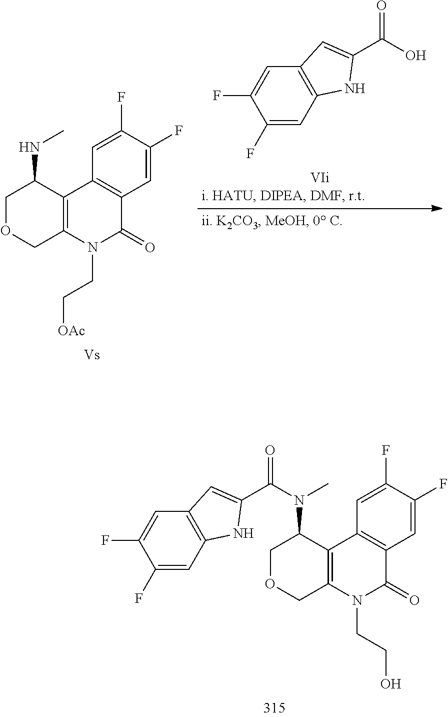 [SMILES: CN[C@@H]1COCc2c1c1cc(F)c(F)cc1c(=O)n2CCOC(C)=O ; O=C(O)c1cc2cc(F)c(F)cc2[nH]1 ; CN(C(=O)c1cc2cc(F)c(F)cc2[nH]1)[C@@H]1COCc2c1c1cc(F)c(F)cc1c(=O)n2CCO]

Step i. Enantiomerically pure (S)-2-(1-(5,6-difluoro-N-methyl-1H-indole-2-carboxamido)-8,9-difluoro-6-oxo-1,2,4,6-tetrahydro-5H-pyrano[3,4-c]isoquinolin-5-yl)ethyl acetate was synthesized in an analogous manner as described above, from (S)-2-(8,9-difluoro-1-(methylamino)-6-oxo-1,2,4,6-tetrahydro-5H-pyrano[3,4-c]isoquinolin-5-yl)ethyl acetate (Vs) and 5,6-difluoro-1H-indole-2-carboxylic acid (VIi). LCMS m/z found 530.1 [M−H]⁻, RT=1.09 min (Method E).

Step ii. To a stirred solution of 65 mg (0.14 mmol, 1 eq.) of (S)-2-(1-(5,6-difluoro-N-methyl-1H-indole-2-carboxamido)-8,9-difluoro-6-oxo-1,2,4,6-tetrahydro-5H-pyrano[3,4-c]isoquinolin-5-yl)ethyl acetate in 1 mL of MeOH was added 41 mg of K₂CO₃ (0.3 mmol, 2 eq.) at 0° C. and the reaction mixture was stirred at room temperature for 2 h. After completion of the reaction (monitored by TLC), the mixture was filtered and the volatiles were evaporated. Preparative HPLC purification afforded 22 mg (26% yield) of (S)-N-(8,9-difluoro-5-(2-hydroxyethyl-6-oxo-1,4,5,6-tetrahydro-2H-pyrano[3,4-c]isoquinolin-1-yl)-5,6-difluoro-N-methyl-1H-indole-2-carboxamide. LCMS m/z found 490.1 [M+H]⁺; RT=4.34 min (Method A); ¹H NMR (400 MHz, DMSO-d₆) δ 11.94 (s, 1H), 8.31 (t, 1H), 7.66-7.59 (m, 2H), 7.39 (t, 1H), 6.96 (s, 1H), 6.02 (s, 1H), 5.00 (t, 1H), 4.82 (d, 1H), 4.69 (d, 1H), 4.49-4.43 (m, 2H), 4.25 (d, 1H), 4.11 (d, 1H), 3.85-3.81 (m, 2H), 3.09 (s, 3H).

### (S)-N-(8,9-Difluoro-6-(2-hydroxyethoxy)-1,4-dihydro-2H-pyrano[3,4-c]isoquinolin-1-yl)-5,6-difluoro-N-methyl-1H-indole-2-carboxamide (Compound 317)

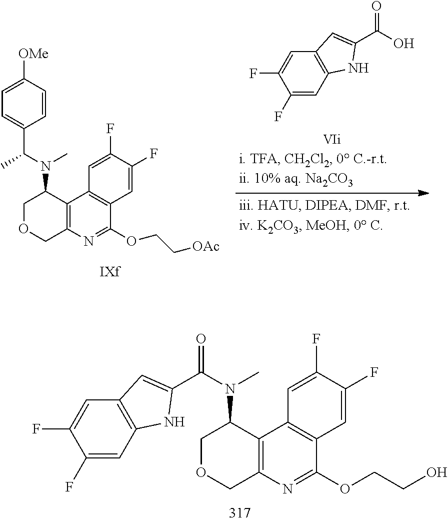 [SMILES: COc1ccc([C@@H](C)N(C)[C@@H]2COCc3nc(OCCOC(C)=O)c4cc(F)c(F)cc4c32)cc1 ; O=C(O)c1cc2cc(F)c(F)cc2[nH]1 ; CN(C(=O)c1cc2cc(F)c(F)cc2[nH]1)[C@@H]1COCc2nc(OCCO)c3cc(F)c(F)cc3c21]

Enantiomerically pure (S)-N-(8,9-difluoro-6-(2-hydroxyethoxy)-1,4-dihydro-2H-pyrano[3,4-c]isoquinolin-1-yl)-5,6-difluoro-N-methyl-1H-indole-2-carboxamide was synthesized in an analogous manner as described above from enantiomerically pure regioisomer 2-(((S)-8,9-difluoro-1-(((R)-1-(4-methoxyphenyl)ethyl)(methyl)amino)-1,4-dihydro-2H-pyrano[3,4-c]isoquinolin-6-yl)oxy)ethyl acetate (IXf) and 5,6-difluoro-1H-indole-2-carboxylic acid (VIi). LCMS m/z found 490.2 [M+H]⁺; RT=4.88 min (Method A); ¹H NMR (400 MHz, DMSO-d₆) δ 11.91 (s, 1H), 8.19 (t, 1H), 7.64-7.59 (m, 1H), 7.46-7.37 (m, 2H), 6.98 (s, 1H), 5.79 (s, 1H), 5.18 (d, 1H), 5.07-5.04 (m, 1H), 4.73 (d, 1H), 4.16 (d, 1H), 4.06-3.92 (m, 3H), 3.74-3.66 (m, 2H), 3.14 (s, 3H).

### 6-Chloro-8,9-difluoro-2H-pyrano[3,4-c]isoquinolin-1(4H)-one (IVl)

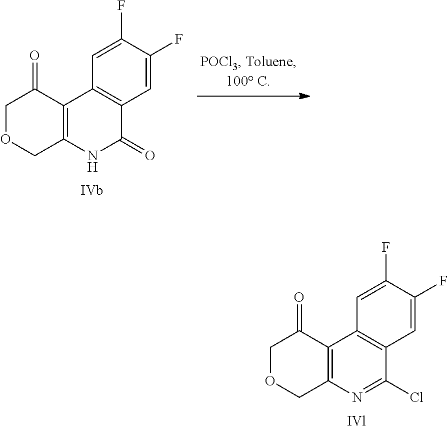 [SMILES: O=C1COCc2[nH]c(=O)c3cc(F)c(F)cc3c21 ; O=C1COCc2nc(Cl)c3cc(F)c(F)cc3c21]

A round bottom flask was charged with 500 mg (1.9 mmol, 1 eq.) of 8,9-difluoro-2H-pyrano[3,4-c]isoquinoline-1,6(4H,5H)-dione (IVb) in 5 mL of Toluene. POCl₃ (1.3 mL, 4 eq.) was added under inert atmosphere. The reaction mixture was stirred for 4 h at 110° C. After completion of reaction (monitored by TLC and LCMS analysis), the mixture was basified with saturated NaHCO₃ solution (50 mL). The solid was filtered and the filtrate was extracted with ethyl acetate (3×200 mL). The combined organic layer was washed with brine (100 mL), dried over anhydrous Na₂SO₄, filtered and concentrated under reduced pressure to afford 480 mg of 6-chloro-8,9-difluoro-2H-pyrano[3,4-c]isoquinolin-1(4H)-one as a pale-yellow solid, which was taken into the next step without purification.

### 8,9-Difluoro-6-(methylamino)-2H-pyrano[3,4-c]isoquinolin-1(4H)-one (IVm)

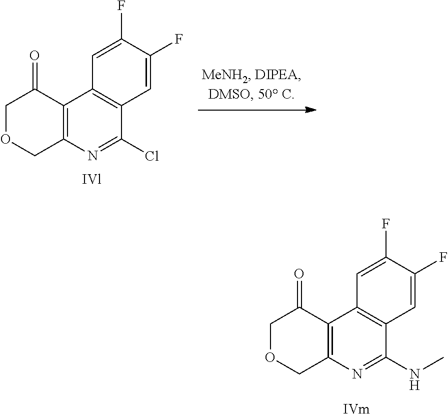 [SMILES: O=C1COCc2nc(Cl)c3cc(F)c(F)cc3c21 ; CNc1nc2c(c3cc(F)c(F)cc13)C(=O)COC2]

To a solution of 400 mg (1.48 mmol, 1 eq.) 6-chloro-8,9-difluoro-2H-pyrano[3,4-c]isoquinolin-1(4H)-one (IVi) in 4 mL of DMSO in a sealed tube, 2.2 mL (4.46 mmol, 3 eq.) of methylamine in THF (1 M) and DIPEA (0.5 mL) were added and the mixture was heated at 50° C. for 16 h. After completion of reaction (monitored by TLC), the mixture was cooled to room temperature and poured into ice cold water (20 mL). then extracted with EtOAc (2×50 mL). The combined organic layer was washed with brine (50 mL), dried over anhydrous Na₂SO₄, filtered and concentrated under reduced pressure. The resulting crude product was triturated with diethyl ether to afford 320 mg (81% yield) of 8,9-difluoro-6-(methylamino)-2H-pyrano[3,4-c]isoquinolin-1(4H)-one as a brown solid. LCMS m/z found 265.3 [M+H]⁺.

### 8,9-Difluoro-N1,N6-dimethyl-1,4-dihydro-2H-pyrano[3,4-c]isoquinoline-1,6-diamine (Vt)

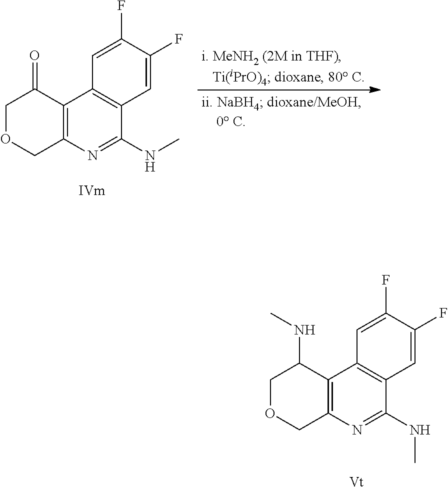 [SMILES: CNc1nc2c(c3cc(F)c(F)cc13)C(=O)COC2 ; CNc1nc2c(c3cc(F)c(F)cc13)C(NC)COC2]

8,9-Difluoro-N1,N6-dimethyl-1,4-dihydro-2H-pyrano[3,4-c]isoquinoline-1,6-diamine was synthesized in an analogous manner as described above, from 8,9-difluoro-6-(methylamino)-2H-pyrano[3,4-c]isoquinolin-1(4H)-one (IVm) and methanamine. LCMS m/z found 280.2 [M+H]⁺.

### N-(8,9-Difluoro-6-(methylamino)-1,4-dihydro-2H-pyrano[3,4-c]isoquinolin-1-yl)-5,6-difluoro-N-methyl-1H-indole-2-carboxamide (Compounds 333 and 334)

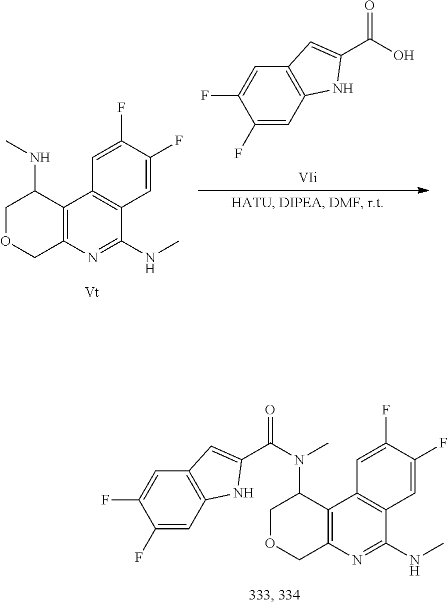 [SMILES: CNc1nc2c(c3cc(F)c(F)cc13)C(NC)COC2 ; O=C(O)c1cc2cc(F)c(F)cc2[nH]1 ; CNc1nc2c(c3cc(F)c(F)cc13)C(N(C)C(=O)c1cc3cc(F)c(F)cc3[nH]1)COC2]

Racemic N-(8,9-difluoro-6-(methylamino)-1,4-dihydro-2H-pyrano[3,4-c]isoquinolin-1-yl)-5,6-difluoro-N-methyl-1H-indole-2-carboxamide was synthesized in an analogous manner as described above, from racemic 8,9-difluoro-N1,N6-dimethyl-1,4-dihydro-2H-pyrano[3,4-c]isoquinoline-1,6-diamine (Vt) and 5,6-difluoro-1H-indole-2-carboxylic acid (VIi). The enantiomers were subsequently separated by preparative SFC: Method isocratic, Mobile phase (0.2% (7 M methanolic ammonia) in Methanol):CO₂—25:75. Column: Chiralpak-IC (30×250 mm) 5 μm, flow rate: 100 g/min.

Enantiomer I (Compound 333): LCMS: m/z found 459.2 [M+H]⁺, RT=3.50 min, (Method A); ¹H NMR (400 MHz, DMSO-d₆) δ 11.91 (br s, 1H), 8.37-8.31 (m, 1H), 7.72 (d, 1H), 7.62-7.57 (m, 1H), 7.50-7.45 (m, 1H), 7.40-7.36 (m, 1H), 6.93 (s, 1H), 5.87 (s, 1H), 4.70 (d, 1H), 4.58 (d, 1H), 4.20 (d, 1H), 4.05 (d, 1H), 3.08 (s, 3H), 2.96 (d, 3H); Chiral analytical SFC: RT=1.91 min, Column: Chiralpak IC-3 (4.6×150 mm) 3 μm, 30% of MeOH, Flow rate: 3.0 g/min.

Enantiomer II (Compound 334): LCMS: m/z found 459.2 [M+H]⁺, RT=3.50 min, (Method A); ¹H NMR (400 MHz, DMSO-d₆) δ 11.91 (br s, 1H), 8.37-8.31 (m, 1H), 7.72 (d, 1H), 7.62-7.57 (m, 1H), 7.50-7.45 (m, 1H), 7.40-7.36 (m, 1H), 6.93 (s, 1H), 5.87 (s, 1H), 4.70 (d, 1H), 4.58 (d, 1H), 4.20 (d, 1H), 4.05 (d, 1H), 3.08 (s, 3H), 2.96 (d, 3H); Chiral analytical SFC: RT=2.38 min, Column: Chiralpak IC-3 (4.6×150 mm) 3 μm, 30% of MeOH, Flow rate: 3.0 g/min.

### N-(8,9-Difluoro-6-((2-hydroxyethyl)amino)-1,4-dihydro-2H-pyrano[3,4-c]isoquinolin-1-yl)-5,6-difluoro-N-methyl-1H-indole-2-carboxamide (Compounds 335 and 336)

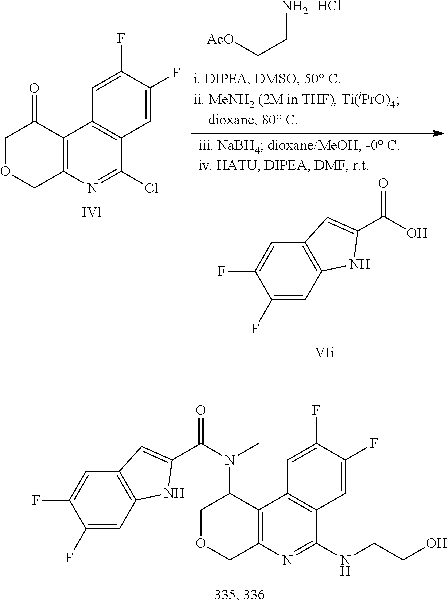 [SMILES: Cl ; O=C1COCc2nc(Cl)c3cc(F)c(F)cc3c21 ; O=C(O)c1cc2cc(F)c(F)cc2[nH]1 ; CC(=O)OCCN ; CN(C(=O)c1cc2cc(F)c(F)cc2[nH]1)C1COCc2nc(NCCO)c3cc(F)c(F)cc3c21]

Racemic N-(8,9-difluoro-6-((2-hydroxyethyl)amino)-1,4-dihydro-2H-pyrano[3,4-c]isoquinolin-1-yl)-5,6-difluoro-N-methyl-1H-indole-2-carboxamide was synthesized in an analogous manner as described above, from 6-chloro-8,9-difluoro-2H-pyrano[3,4-c]isoquinolin-1(4H)-one (IVi), 2-aminoethyl acetate hydrochloride, methanamine, and 5,6-difluoro-1H-indole-2-carboxylic acid (VIi). The enantiomers were subsequently separated by preparative SFC: Method isocratic, Mobile phase (0.2% (7 M methanolic ammonia) in Methanol):CO₂—25:75. Column: ChiralpakIC-3 (30×250 mm), 5μ, flow rate: 100 g/min.

Enantiomer I (Compound 335): LCMS: m/z found 489.2 [M+H]⁺, RT=3.46 min, (Method A); ¹H NMR (400 MHz, DMSO-d₆) δ 11.91 (br s, 1H), 8.46-8.41 (m, 1H), 7.68-7.65 (m, 1H), 7.62-7.54 (m, 1H), 7.50-7.44 (m, 1H), 7.40-7.35 (m, 1H), 6.93 (s, 1H), 5.86 (s, 1H), 4.76 (bs, 1H), 4.68 (d, 1H), 4.56 (d, 1H), 4.19 (d, 1H), 4.05 (d, 1H), 3.63-3.52 (m, 4H), 3.09 (s, 3H); Chiral analytical SFC: RT=2.35 min, Column: Chiralpak IC-3 (4.6×150 mm) 3 μm, 30% of MeOH, Flow rate: 3.0 g/min.

Enantiomer II (Compound 336): LCMS: m/z found 489.2 [M+H]⁺, RT=3.46 min, (Method A); ¹H NMR (400 MHz, DMSO-d₆) δ 11.91 (br s, 1H), 8.46-8.41 (m, 1H), 7.68-7.65 (m, 1H), 7.62-7.54 (m, 1H), 7.50-7.44 (m, 1H), 7.40-7.35 (m, 1H), 6.93 (s, 1H), 5.86 (s, 1H), 4.76 (bs, 1H), 4.68 (d, 1H), 4.56 (d, 1H), 4.19 (d, 1H), 4.05 (d, 1H), 3.63-3.52 (m, 4H), 3.09 (s, 3H); Chiral analytical SFC: RT=3.62 min, Column: Chiralpak IC-3 (4.6×150 mm) 3 μm, 30% of MeOH, Flow rate: 3.0 g/min.

### N-(6-((2-Aminoethyl)amino)-8,9-difluoro-1,4-dihydro-2H-pyrano[3,4-c]isoquinolin-1-yl)-5,6-difluoro-N-methyl-1H-indole-2-carboxamide (Compounds 351 and 352)

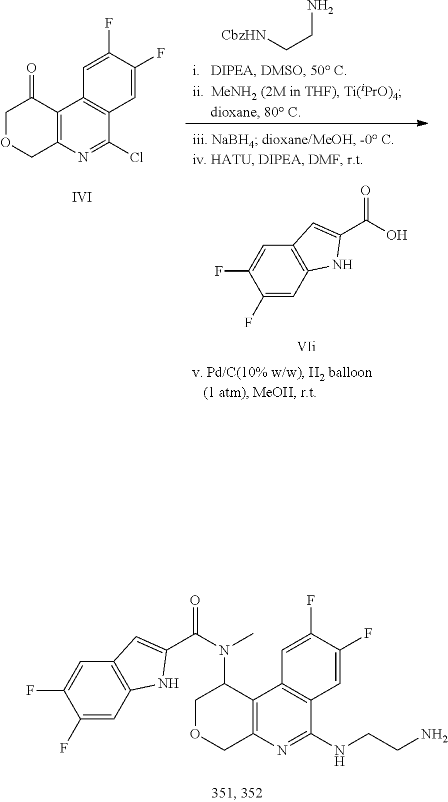

Racemic benzyl(2-((1-(5,6-difluoro-N-methyl-1H-indole-2-carboxamido)-8,9-difluoro-1,4-dihydro-2H-pyrano[3,4-c]isoquinolin-6-yl)amino)ethyl)carbamate was synthesized in an analogous manner as described above, from 6-chloro-8,9-difluoro-2H-pyrano[3,4-c]isoquinolin-1(4H)-one (IVi), benzyl (2-aminoethyl)carbamate, methanamine, and 5,6-difluoro-1H-indole-2-carboxylic acid (VIi). To a stirred solution of 140 mg (0.19 mmol, 1 eq.) crude benzyl(2-((1-(5,6-difluoro-N-methyl-1H-indole-2-carboxamido)-8,9-difluoro-1,4-dihydro-2H-pyrano[3,4-c]isoquinolin-6-yl)amino)ethyl)carbamate in 10 mL of MeOH was added 50 mg (0.11 mmol, 0.6 eq.) Pd/C and the reaction was stirred for 6 h. After completion of the reaction (monitored by TLC), the mixture was filtered through a pad of CELITE®, which was further washed with MeOH (30 mL). Combined filtrate was concentrated under reduced pressure to afford 75 mg (32% yield) of racemic N-(6-((2-aminoethyl)amino)-8,9-difluoro-1,4-dihydro-2H-pyrano[3,4-c]isoquinolin-1-yl)-5,6-difluoro-N-methyl-1H-indole-2-carboxamide. The enantiomers were subsequently separated by preparative SFC: Method isocratic, Mobile phase Methanol:CO₂—25:75. Column: Chiralpak OX-3 (30×250 mm), 5 μm, flow rate: 100 g/min.

Enantiomer I (Compound 351): LCMS: m/z found 488.2 [M+H]⁺, RT=3.50 min, (Method A); ¹H NMR (400 MHz, DMSO-d₆) δ 11.78 (br s, 1H), 8.49-8.44 (m, 1H), 7.63-7.58 (m, 2H), 7.48-7.36 (m, 2H), 6.93 (s, 1H), 5.86 (s, 1H), 4.70-4.54 (m, 2H), 4.25-4.00 (m, 2H), 3.46 (m, 2H), 3.08 (s, 3H), 2.81 (t, 2H); Chiral analytical SFC: RT=2.32 min, Column: Chiralpak OX-3 (4.6×150 mm) 3 μm, 40% (0.5% DEA in Methanol), Flow rate: 3.0 g/min.

Enantiomer II (Compound 352): LCMS: m/z found 488.2 [M+H]⁺, RT=3.50 min, (Method A); ¹H NMR (400 MHz, DMSO-d₆) δ 11.78 (br s, 1H), 8.49-8.44 (m, 1H), 7.63-7.58 (m, 2H), 7.48-7.36 (m, 2H), 6.93 (s, 1H), 5.86 (s, 1H), 4.70-4.54 (m, 2H), 4.25-4.00 (m, 2H), 3.46 (m, 2H), 3.08 (s, 3H), 2.81 (t, 2H); Chiral analytical SFC: RT=3.45 min, Column: Chiralpak OX-3 (4.6×150 mm) 3 μm, 40% (0.5% DEA in Methanol), Flow rate: 3.0 g/min.

### (S)-N-(8,9-Difluoro-6-oxo-1,4,5,6-tetrahydro-2H-pyrano[3,4-c]isoquinolin-1-yl)-5-fluoro-N-methyl-1H-pyrrolo[2,3-b]pyridine-2-carboxamide (Compound 190)

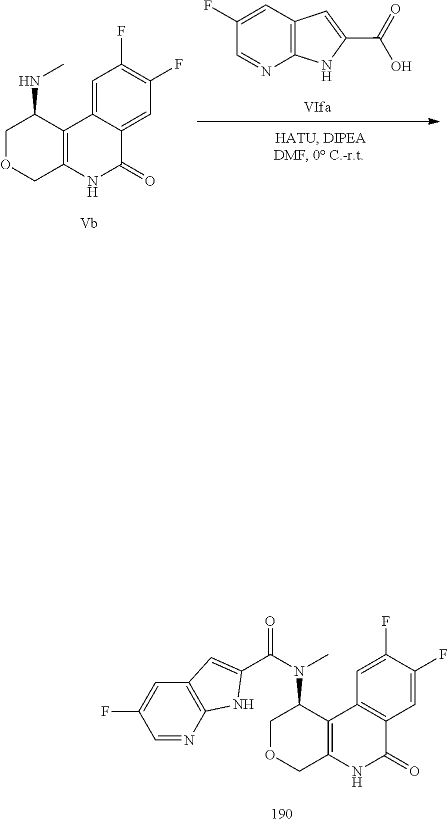

Enantiomerically pure (S)-N-(8,9-difluoro-6-oxo-1,4,5,6-tetrahydro-2H-pyrano[3,4-c]isoquinolin-1-yl)-5-fluoro-N-methyl-1H-pyrrolo[2,3-b]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-fluoro-1H-pyrrolo[2,3-b]pyridine-2-carboxylic acid (VIfa). LCMS m/z found 429.3 [M+H]⁺; RT=2.23 min (Method D); ¹H NMR (400 MHz, Chloroform-d) δ 10.58 (s, 1H), 10.04 (s, 1H), 8.39 (t, 1H), 8.23 (dd, 1H), 7.67 (dd, 1H), 7.43 (dd, 1H), 6.84 (d, 1H), 5.90 (s, 1H), 4.77 (d, 1H), 4.65 (d, 1H), 4.39 (d, 1H), 4.08 (dd, 1H), 3.33 (s, 3H).

### (S)-5-Chloro-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide (Compound 191)

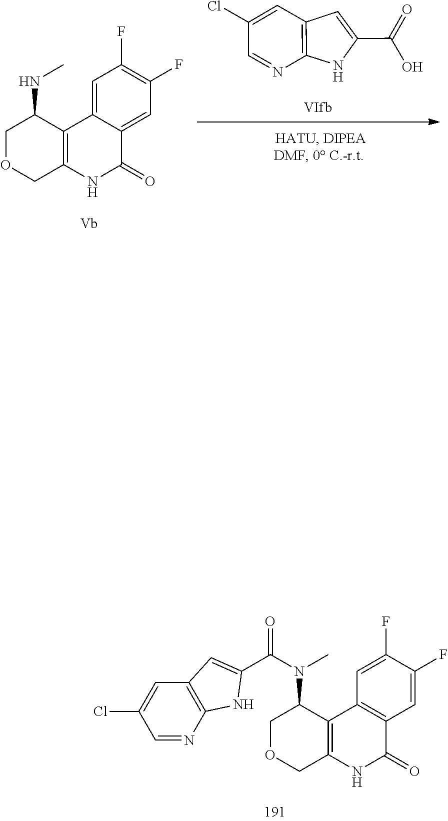

Enantiomerically pure (S)-5-chloro-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-chloro-1H-pyrrolo[2,3-b]pyridine-2-carboxylic acid (VIfb). LCMS m/z found 445.3 [M+H]⁺; RT=2.48 min (Method D); ¹H NMR (400 MHz, Chloroform-d) δ 10.10 (s, 1H), 9.92 (s, 1H), 8.43 (d, 1H), 8.22 (dd, 1H), 7.96 (d, 1H), 7.42 (dd, 1H), 6.81 (d, 1H), 5.89 (s, 1H), 4.74 (d, 1H), 4.63 (d, 1H), 4.39 (d, 1H), 4.08 (dd, 1H), 3.32 (s, 3H).

### (S)-5-Bromo-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide (Compound 192)

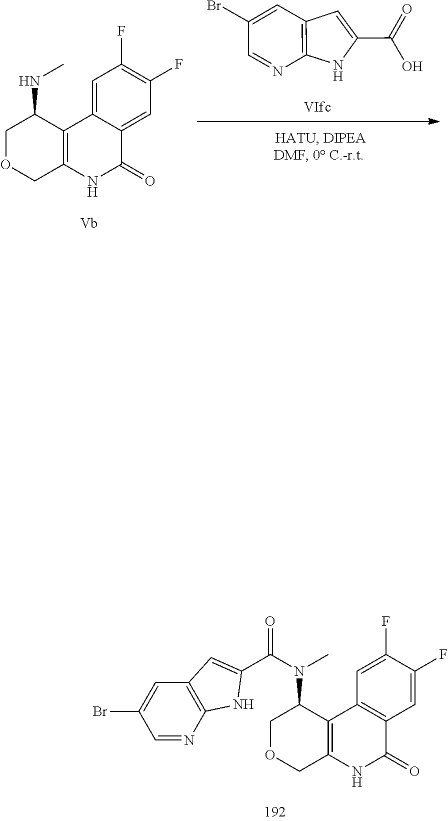

Enantiomerically pure (S)-5-bromo-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-bromo-1H-pyrrolo[2,3-b]pyridine-2-carboxylic acid (VIfc). LCMS m/z found 489.2 [M+H]⁺; RT=2.55 min (Method D); ¹H NMR (400 MHz, Chloroform-d) δ 10.76 (s, 1H), 10.15 (s, 1H), 8.52 (d, 1H), 8.23 (dd, 1H), 8.12 (d, 1H), 7.43 (dd, 1H), 6.80 (d, 1H), 5.90 (s, 1H), 4.78 (d, 1H), 4.65 (d, 1H), 4.40 (d, 1H), 4.08 (dd, 1H), 3.32 (s, 3H).

### (S)-N-(8,9-Difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-3-carboxamide (Compound 193)

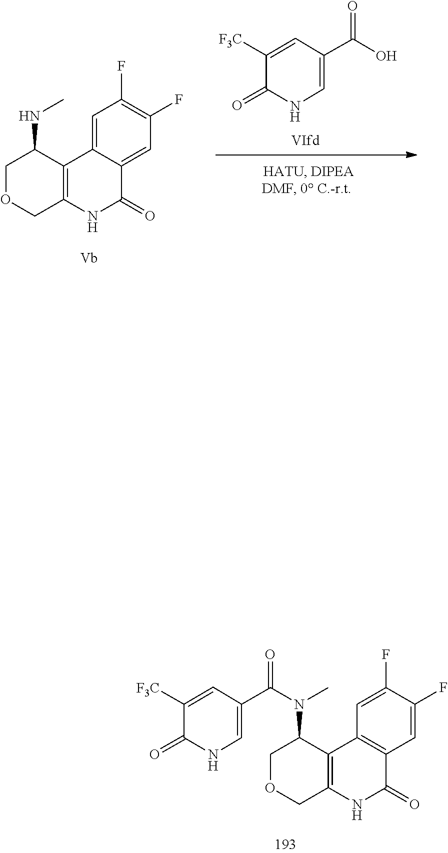

Enantiomerically pure (S)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-3-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 6-oxo-5-(trifluoromethyl)-1H-pyridine-3-carboxylic acid (VIfd). LCMS m/z found 456.2 [M+H]⁺; RT=1.97 min (Method D); ¹H NMR (400 MHz, DMSO-d₆) δ 12.61 (s, 1H), 11.69 (s, 1H), 8.10 (dd, 1H), 7.98 (s, 2H), 7.47 (s, 1H), 5.55 (s, 1H), 4.57 (d, 1H), 4.43 (d, 1H), 4.27 (d, 1H), 3.93 (dd, 1H), 2.84 (s, 3H).

### (S)-5-Chloro-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide (Compound 201)

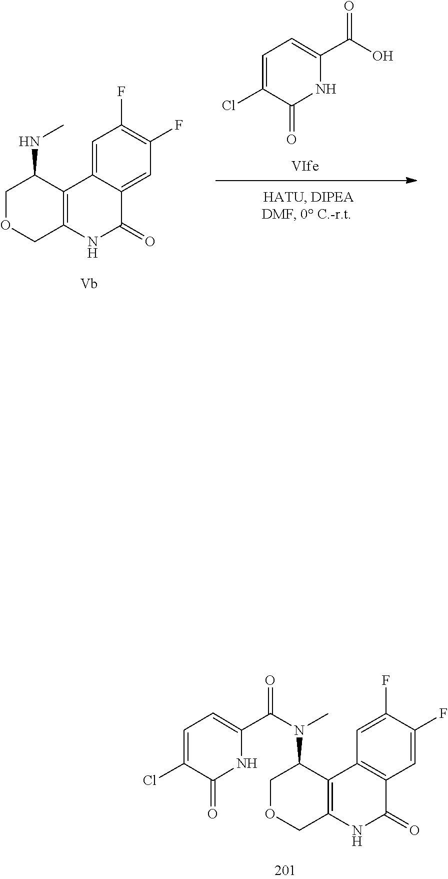

Enantiomerically pure (S)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-chloro-6-oxo-1H-pyridine-2-carboxylic acid (VIfe). LCMS m/z found 422.2 [M+H]⁺; RT=1.82 min (Method D); ¹H NMR (400 MHz, DMSO-d₆) δ 12.77 (s, 1H), 11.70 (s, 1H), 8.11 (dd, 1H), 7.80-7.69 (m, 1H), 7.39 (dd, 1H), 6.30-6.13 (m, 1H), 5.59-5.46 (m, 1H), 4.57 (d, 1H), 4.44 (d, 1H), 4.32-4.23 (m, 1H), 3.93 (d, 1H), 2.72 (s, 3H).

### (S)-5-Chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide (Compound 204)

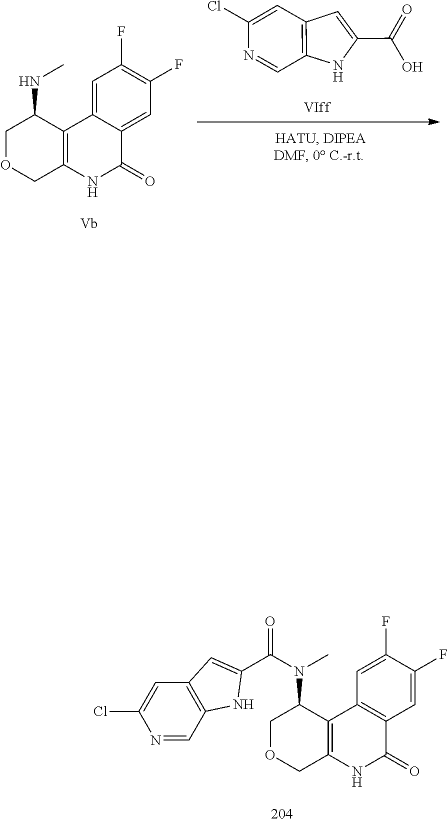

Enantiomerically pure (S)-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-chloro-1H-pyrrolo[2,3-c]pyridine-2-carboxylic acid (VIff). LCMS m/z found 445.2 [M+H]⁺; RT=2.25 min (Method D); ¹H NMR (400 MHz, DMSO-d₆) δ 12.42 (s, 1H), 11.74 (s, 1H), 8.60 (s, 1H), 8.12 (dd, 1H), 7.67 (s, 1H), 7.41 (dd, 1H), 6.93 (s, 1H), 5.72 (s, 1H), 4.62 (d, 1H), 4.47 (d, 1H), 4.19 (d, 1H), 4.03 (dd, 1H), 3.09 (s, 3H).

### (S)-5-Bromo-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide (Compound 205)

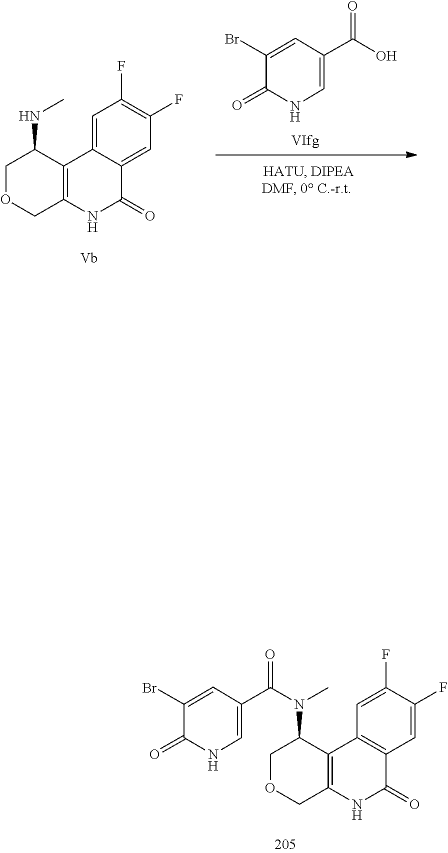

Enantiomerically pure (S)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-bromo-6-oxo-1H-pyridine-3-carboxylic acid (VIfg). LCMS m/z found 466.1 [M+H]⁺; RT=1.86 min (Method D); ¹H NMR (400 MHz, DMSO-d₆) δ 12.44 (s, 1H), 11.70 (s, 1H), 8.12 (dd, 1H), 8.03 (d, 1H), 7.73 (s, 1H), 7.46 (s, 1H), 5.55 (s, 1H), 4.58 (d, 1H), 4.44 (d, 1H), 4.25 (d, 1H), 3.95 (dd, 1H), 2.85 (s, 3H).

### (S)-5-Chloro-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide (Compound 206)

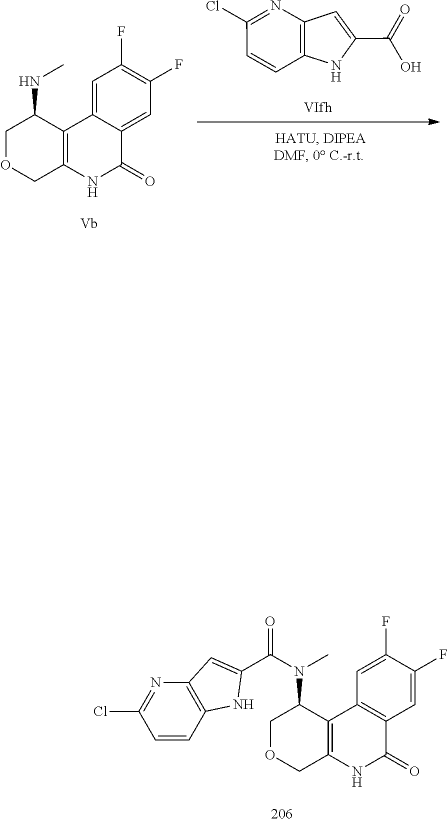

Enantiomerically pure (S)-5-chloro-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-chloro-1H-pyrrolo[3,2-b]pyridine-2-carboxylic acid (VIfh). LCMS m/z found 445.2 [M+H]⁺; RT=2.45 min (Method D); ¹H NMR (400 MHz, DMSO-d₆) δ 12.24 (s, 1H), 11.76 (s, 1H), 8.13 (dd, 1H), 7.89 (d, 1H), 7.44 (dd, 1H), 7.28 (d, 1H), 7.06 (s, 1H), 5.76 (d, 1H), 4.65 (d, 1H), 4.49 (d, 1H), 4.20 (d, 1H), 4.04 (dd, 1H), 3.15 (s, 3H).

### (S)-5-Bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide (Compound 213)

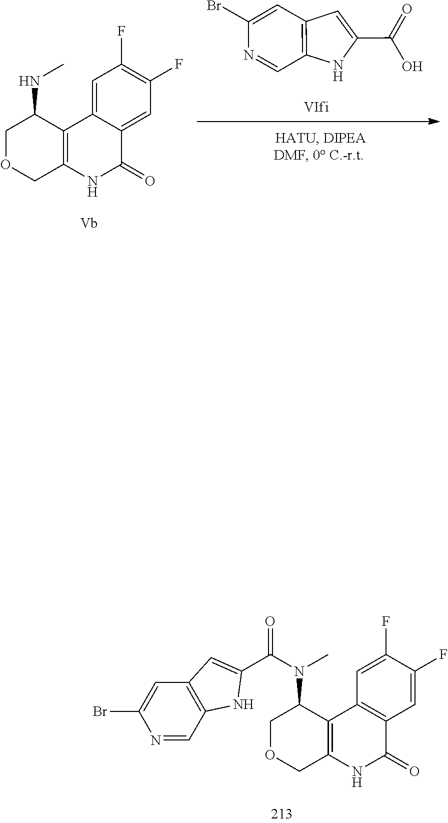

Enantiomerically pure (S)-5-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-bromo-1H-pyrrolo[2,3-c]pyridine-2-carboxylic acid (VIfi). LCMS m/z found 489.2 [M+H]⁺; RT=2.47 min (Method D; ¹H NMR (400 MHz, DMSO-d₆) δ 12.44 (s, 1H), 11.76 (s, 1H), 8.61 (s, 1H), 8.23-8.02 (m, 1H), 7.83 (s, 1H), 7.43 (dd, 1H), 6.94 (s, 1H), 5.74 (s, 1H), 4.64 (d, 1H), 4.49 (d, 1H), 4.21 (d, 1H), 4.04 (d, 1H), 3.11 (s, 3H).

### (S)-N-(8,9-Difluoro-6-oxo-1,4,5,6-tetrahydro-2H-pyrano[3,4-c]isoquinolin-1-yl)-N-methyl-5-(trifluoromethyl)-1¹H-pyrrolo[2,3-b]pyridine-2-carboxamide (Compound 214)

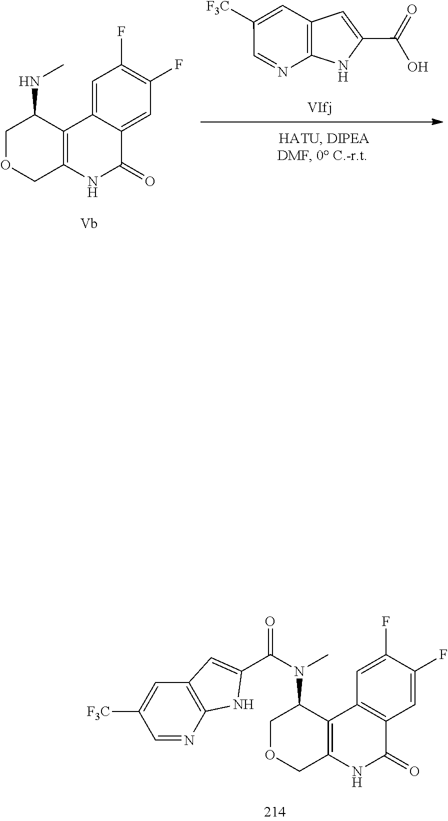

Enantiomerically pure (S)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-b]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-(trifluoromethyl)-1H-pyrrolo[2,3-b]pyridine-2-carboxylic acid (VIfj). LCMS m/z found 479.2 [M+H]⁺; RT=2.70 min (Method D); ¹H NMR (400 MHz, DMSO-d₆) δ 12.87 (s, 1H), 11.77 (s, 1H), 8.69 (s, 1H), 8.48 (s, 1H), 8.18-8.11 (m, 1H), 7.43 (dd, 1H), 7.03 (d, 1H), 5.71 (s, 1H), 4.64 (d, 1H), 4.49 (d, 1H), 4.22 (d, 1H), 4.05 (d, 1H), 3.08 (s, 3H).

### (S)-5-Chloro-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide (Compound 215)

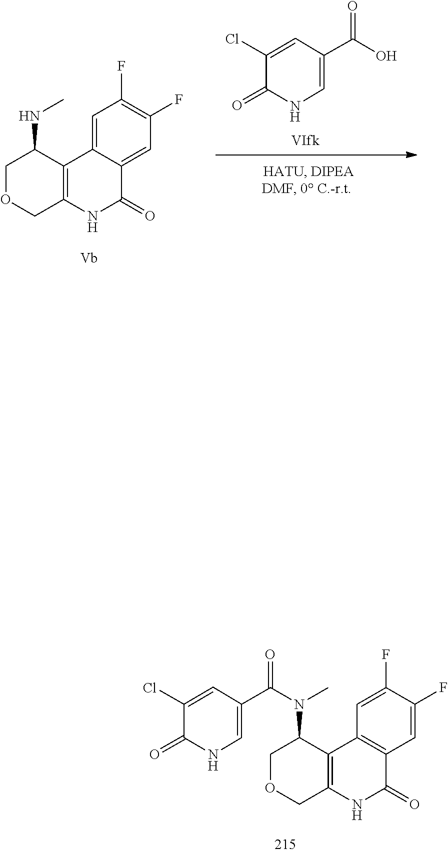

Enantiomerically pure (S)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-chloro-6-oxo-1H-pyridine-3-carboxylic acid (VIfk). LCMS m/z found 422.2 [M+H]⁺; RT=1.80 min (Method D); ¹H NMR (400 MHz, DMSO-d₆) δ 12.49 (s, 1H), 11.71 (s, 1H), 8.17-8.06 (m, 1H), 7.86 (s, 1H), 7.70 (s, 1H), 7.51-7.40 (m, 1H), 5.55 (s, 1H), 4.58 (d, 1H), 4.44 (d, 1H), 4.25 (d, 1H), 3.96 (dd, 1H), 2.85 (s, 3H).

### (S)-5-Bromo-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide (Compound 216)

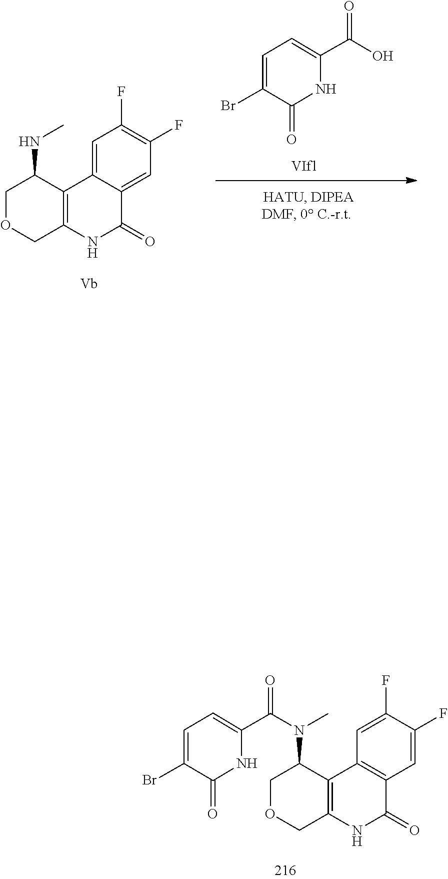

Enantiomerically pure (S)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-chloro-6-oxo-1H-pyridine-3-carboxylic acid (VIfl). LCMS m/z found 466.1 [M+H]⁺; RT=2.06 min (Method D); ¹H NMR (400 MHz, DMSO-d₆) δ 12.75 (s, 1H), 11.72 (s, 1H), 8.13 (dd, 1H), 8.03-7.90 (m, 1H), 7.41 (dd, 1H), 6.22-6.08 (m, 1H), 5.53 (s, 1H), 4.59 (d, 1H), 4.45 (d, 1H), 4.29 (d, 1H), 3.95 (d, 1H), 2.74 (s, 3H).

### (S)-N-(8,9-Difluoro-6-oxo-1,4,5,6-tetrahydro-2H-pyrano[3,4-c]isoquinolin-1-yl)-N-methyl-5-(trifluoromethyl)-1¹H-pyrrolo[3,2-b]pyridine-2-carboxamide (Compound 219)

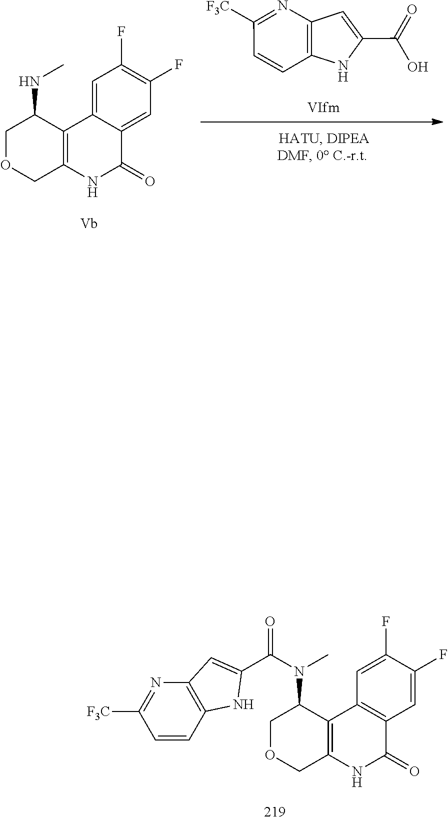

Enantiomerically pure (S)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[3,2-b]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-(trifluoromethyl)-1H-pyrrolo[3,2-b]pyridine-2-carboxylic acid (VIfm). LCMS m/z found 479.2 [M+H]⁺; RT=2.80 min (Method D); ¹H NMR (400 MHz, DMSO-d₆) δ 12.44 (s, 1H), 11.77 (s, 1H), 8.14 (dd, 1H), 8.07 (d, 1H), 7.70 (d, 1H), 7.45 (dd, 1H), 7.27 (s, 1H), 5.77 (s, 1H), 4.66 (d, 1H), 4.50 (d, 1H), 4.22 (d, 1H), 4.06 (dd, 1H), 3.17 (s, 3H).

### (S)-N-(8,9-Difluoro-6-oxo-1,4,5,6-tetrahydro-2H-pyrano[3,4-c]isoquinolin-1-yl)-5-fluoro-N-methyl-6-oxo-1,6-dihydropyridine-3-carboxamide (Compound 226)

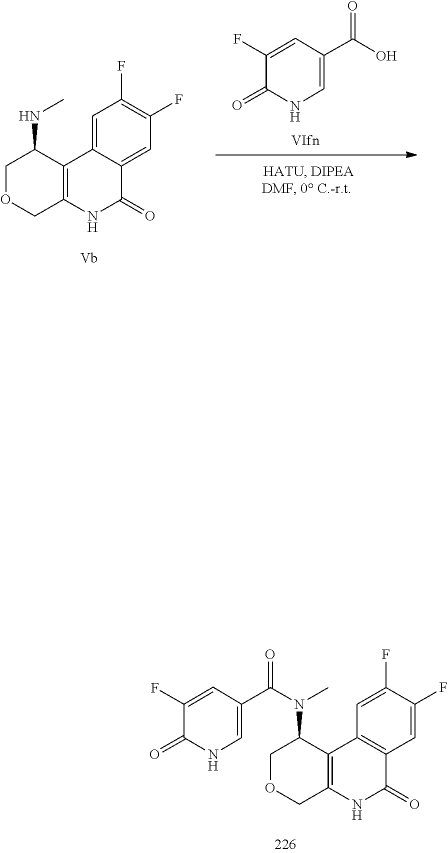

Enantiomerically pure (S)-N-(8,9-difluoro-6-oxo-1,4,5,6-tetrahydro-2H-pyrano[3,4-c]isoquinolin-1-yl)-5-fluoro-N-methyl-6-oxo-1,6-dihydropyridine-3-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-fluoro-6-oxo-1H-pyridine-3-carboxylic acid (VIfn). LCMS m/z found 406.3 [M+H]⁺; RT=1.90 min (Method D); ¹H NMR (400 MHz, DMSO-d₆) δ 12.45 (s, 1H), 11.70 (s, 1H), 8.12 (dd, 1H), 7.54 (d, 2H), 7.43 (s, 1H), 5.55 (s, 1H), 4.59 (d, 1H), 4.44 (d, 1H), 4.23 (d, 1H), 4.00-3.92 (m, 1H), 2.85 (s, 3H).

### (S)-N-(8,9-Difluoro-6-oxo-1,4,5,6-tetrahydro-2H-pyrano[3,4-c]isoquinolin-1-yl)-5-fluoro-N-methyl-1H-pyrrolo[3,2-b]pyridine-2-carboxamide (Compound 232)

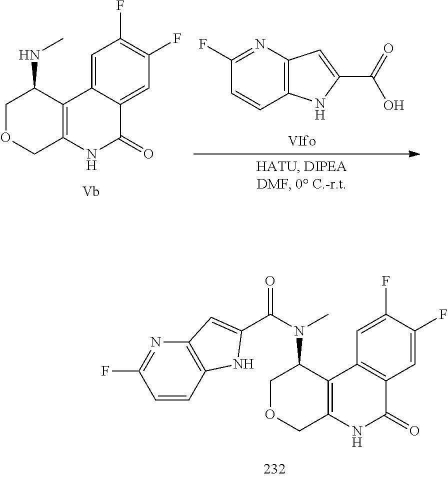

Enantiomerically pure (S)-N-(8,9-difluoro-6-oxo-1,4,5,6-tetrahydro-2H-pyrano[3,4-c]isoquinolin-1-yl)-5-fluoro-N-methyl-1H-pyrrolo[3,2-b]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-fluoro-1H-pyrrolo[3,2-b]pyridine-2-carboxylic acid (VIfo). LCMS m/z found 429.3 [M+H]⁺; RT=2.89 min (Method D); ¹H NMR (400 MHz, DMSO-d₆) δ 12.17 (s, 1H), 11.73 (s, 1H), 8.11 (dd, 1H), 7.98 (t, 1H), 7.43 (dd, 1H), 7.02-6.95 (m, 2H), 5.74 (s, 1H), 4.63 (d, 1H), 4.47 (d, 1H), 4.17 (d, 1H), 4.03 (dd, 1H), 3.14 (s, 3H).

### (S)-N-(8,9-Difluoro-6-oxo-1,4,5,6-tetrahydro-2H-pyrano[3,4-c]isoquinolin-1-yl)-5-hydroxy-N-methyl-6-oxo-1,6-dihydropyridine-2-carboxamide (Compound 243)

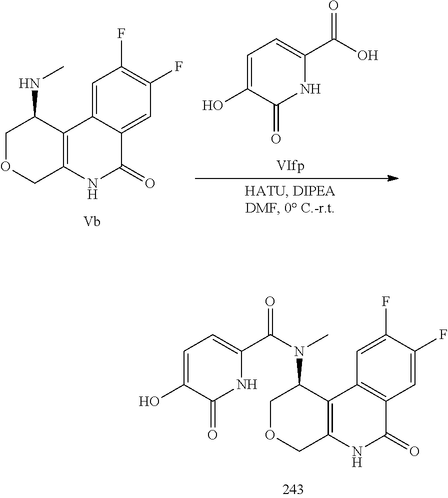 [SMILES: CN[C@@H]1COCc2[nH]c(=O)c3cc(F)c(F)cc3c21 ; O=C(O)c1ccc(O)c(=O)[nH]1 ; CN(C(=O)c1ccc(O)c(=O)[nH]1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-5-hydroxy-N-methyl-6-oxo-1,6-dihydropyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-hydroxy-6-oxo-1,6-dihydropyridine-2-carboxylic acid (VIfp). LCMS m/z found 404.2 [M+H]⁺; RT=2.51 min (Method D); ¹H NMR (400 MHz, DMSO-d₆) δ 12.10 (s, 1H), 11.71 (s, 1H), 9.52 (s, 1H), 8.12 (dd, 1H), 7.50-7.34 (m, 1H), 6.70 (s, 1H), 6.21 (s, 1H), 5.54 (s, 1H), 4.59 (d, 1H), 4.45 (d, 1H), 4.24 (d, 1H), 3.95 (d, 1H), 2.78 (s, 3H).

### (S)-4-Bromo-N-(8,9-difluoro-6-oxo-1,4,5,6-tetrahydro-2H-pyrano[3,4-c]isoquinolin-1-yl)-2,3-difluoro-N-methylbenzamide (Compound 244)

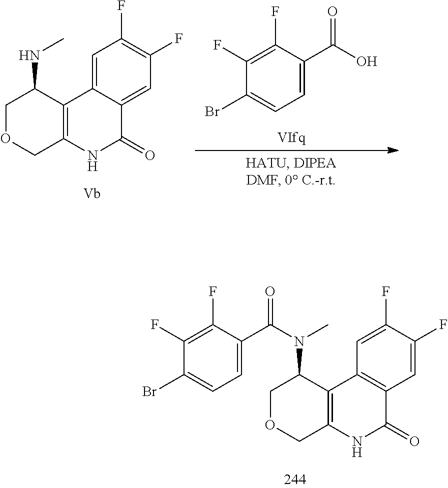

Enantiomerically pure (S)-4-bromo-N-(8,9-difluoro-6-oxo-1,4,5,6-tetrahydro-2H-pyrano[3,4-c]isoquinolin-1-yl)-2,3-difluoro-N-methylbenzamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 4-bromo-2,3-difluoro-benzoic acid (VIfq). LCMS m/z found 485.1 [M+H]⁺; RT=3.56 min (Method D); ¹H NMR (400 MHz, DMSO-d₆) δ 11.73 (s, 1H), 8.14 (dd, 1H), 7.70-7.64 (m, 1H), 7.40 (dd, 1H), 7.36-7.30 (m, 1H), 5.65 (s, 1H), 4.59 (d, 1H), 4.47 (d, 1H), 4.25 (d, 1H), 4.03 (dd, 1H), 2.67 (s, 3H).

### (S)-4-Bromo-N-(8,9-difluoro-6-oxo-1,4,5,6-tetrahydro-2H-pyrano[3,4-c]isoquinolin-1-yl)-2,5-difluoro-N-methylbenzamide (Compound 245)

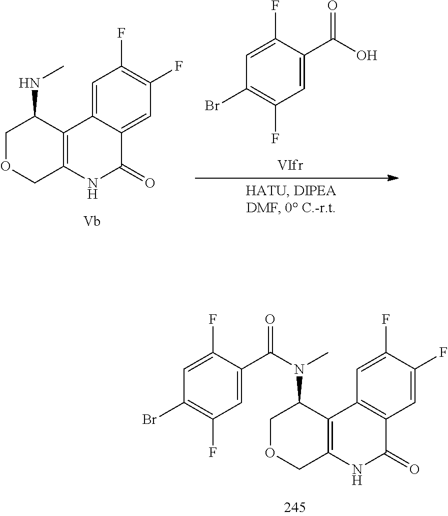 [SMILES: CN[C@@H]1COCc2[nH]c(=O)c3cc(F)c(F)cc3c21 ; O=C(O)c1cc(F)c(Br)cc1F ; CN(C(=O)c1cc(F)c(Br)cc1F)[C@@H]1COCc2[nH]c(=O)c3cc(F)c(F)cc3c21]

Enantiomerically pure (S)-4-bromo-N-(8,9-difluoro-6-oxo-1,4,5,6-tetrahydro-2H-pyrano[3,4-c]isoquinolin-1-yl)-2,5-difluoro-N-methylbenzamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 4-bromo-2,5-difluoro-benzoic acid (VIfr). LCMS m/z found 485.1 [M+H]⁺; RT=3.56 min (Method D); ¹H NMR (400 MHz, DMSO-d₆) δ 11.73 (s, 1H), 8.13 (dd, 1H), 7.88 (dd, 1H), 7.75-7.65 (m, 1H), 7.38 (dd, 1H), 5.62 (s, 1H), 4.59 (d, 1H), 4.46 (d, 1H), 4.25 (d, 1H), 4.02 (dd, 1H), 2.65 (s, 3H).

### (S)-4-Bromo-N-(8,9-difluoro-6-oxo-1,4,5,6-tetrahydro-2H-pyrano[3,4-c]isoquinolin-1-yl)-2,6-difluoro-N-methylbenzamide (Compound 246)

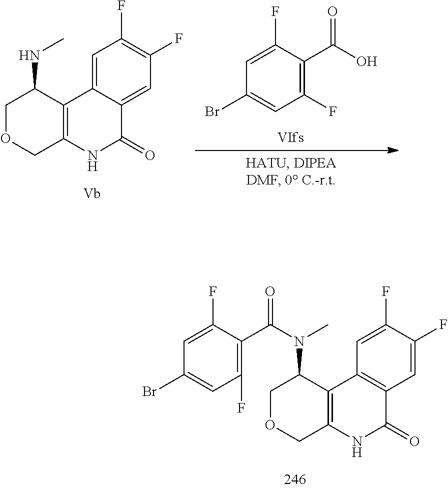 [SMILES: CN[C@@H]1COCc2[nH]c(=O)c3cc(F)c(F)cc3c21 ; O=C(O)c1c(F)cc(Br)cc1F ; CN(C(=O)c1c(F)cc(Br)cc1F)[C@@H]1COCc2[nH]c(=O)c3cc(F)c(F)cc3c21]

Enantiomerically pure (S)-4-bromo-N-(8,9-difluoro-6-oxo-1,4,5,6-tetrahydro-2H-pyrano[3,4-c]isoquinolin-1-yl)-2,6-difluoro-N-methylbenzamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 4-bromo-2,6-difluoro-benzoic acid (VIfs). LCMS m/z found 485.1 [M+H]⁺; RT=3.55 min (Method D); ¹H NMR (400 MHz, DMSO-d₆) δ 11.75 (s, 1H), 8.14 (dd, 1H), 7.72-7.62 (m, 2H), 7.33 (dd, 1H), 5.68-5.63 (m, 1H), 4.60 (d, 1H), 4.48 (d, 1H), 4.10-4.05 (m, 2H), 2.73-2.63 (m, 3H).

### (S)-N-(8,9-Difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-2-carboxamide (Compound 247)

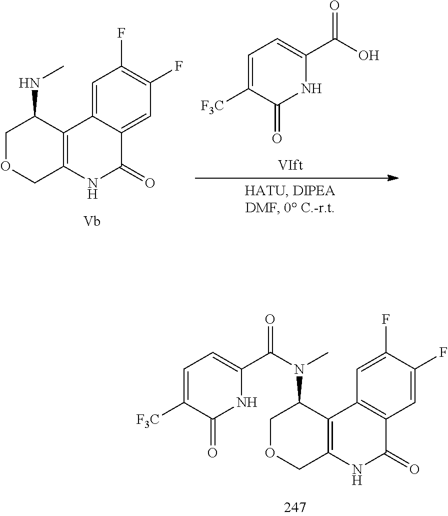 [SMILES: CN[C@@H]1COCc2[nH]c(=O)c3cc(F)c(F)cc3c21 ; O=C(O)c1ccc(C(F)(F)F)c(=O)[nH]1 ; CN(C(=O)c1ccc(C(F)(F)F)c(=O)[nH]1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 6-oxo-5-(trifluoromethyl)-1H-pyridine-2-carboxylic acid (VIft). LCMS m/z found 456.2 [M+H]⁺; RT=2.92 min (Method D); ¹H NMR (400 MHz, DMSO-d₆) δ 13.03 (s, 1H), 11.74 (s, 1H), 8.13 (dd, 1H), 7.98 (d, 1H), 7.46-7.36 (m, 1H), 6.31 (d, 1H), 5.54 (s, 1H), 4.60 (d, 1H), 4.46 (d, 1H), 4.31 (d, 1H), 3.95 (d, 1H), 2.74 (s, 3H).

### 5-(Trifluoromethyl)-1¹H-pyrrolo[2,3-c]pyridine-2-carboxylic acid (VIfu)

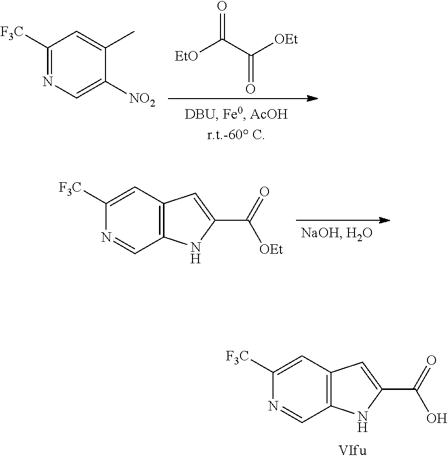 [SMILES: Cc1cc(C(F)(F)F)ncc1[N+](=O)[O-] ; CCOC(=O)C(=O)OCC ; CCOC(=O)c1cc2cc(C(F)(F)F)ncc2[nH]1 ; O=C(O)c1cc2cc(C(F)(F)F)ncc2[nH]1]

Step i. A mixture of 4-methyl-5-nitro-2-(trifluoromethyl)pyridine (0.22 g, 1.1 mmol) and diethyl oxalate (0.65 mL, 4.8 mmol) was stirred at room temperature then DBU (0.38 mL, 2.6 mmol) was added dropwise. The resulting dark, red-purple mixture was stirred at room temperature for 18 hours. The mixture was evaporated under reduced pressure. The mixture was diluted with AcOH (10 mL) and warmed to 60° C. under nitrogen then powdered iron (0.12 g, 2.1 mmol) was added to the vigorously stirred solution. The reaction mixture was heated at 70° C. under nitrogen overnight. The suspension was cooled to room temperature and diluted with water (50 mL). The solid was filtered, rinsed with water (2×25 mL) and partially dried by suction. The solid was taken up in EtOAc (50 mL), dried over Na₂SO₄ and filtered through plug of CELITE®. The filtrate was evaporated under reduced pressure to give ethyl 5-(trifluoromethyl)-1H-pyrrolo[2,3-c]pyridine-2-carboxylate (0.24 g, 87% yield) as a brown solid. LCMS m/z found 259.2 [M+H]⁺; RT=1.00 min (Method B); ¹H NMR (400 MHz, DMSO-d₆) δ 12.93 (s, 1H), 8.95 (s, 1H), 8.23 (s, 1H), 7.36 (s, 1H), 4.40 (q, 2H), 1.36 (t, 3H).

Step ii. A suspension of ethyl 5-(trifluoromethyl)-1H-pyrrolo[2,3-c]pyridine-2-carboxylate (0.24 g, 0.93 mmol) in aq. NaOH solution (1M, 4 mL) was stirred at room temperature for 2 hours. The reaction mixture was cooled to 5° C. and acidified with aq. HCl solution (1M, ˜5 mL). The mixture was stirred for 10 minutes then extracted with EtOAc (50 mL). The layers were separated. The organic layer was dried over Na₂SO₄, filtered and evaporated then subjected to high vacuum for 1 h to give 5-(trifluoromethyl)-1H-pyrrolo[2,3-c]pyridine-2-carboxylic acid (VIfu) (0.21 g, 97% yield) as a light-brown solid. LCMS m/z found 231.1 [M+H]⁺; RT=0.97 min (Method B); ¹H NMR (400 MHz, DMSO-d₆) δ 13.72 (s, 1H), 12.80 (s, 1H), 8.92 (s, 1H), 8.22 (s, 1H), 7.29 (d, 1H).

### (S)-N-(8,9-Difluoro-6-oxo-1,4,5,6-tetrahydro-2H-pyrano[3,4-c]isoquinolin-1-yl)-N-methyl-5-(trifluoromethyl)-1¹H-pyrrolo[2,3-c]pyridine-2-carboxamide (Compound 251)

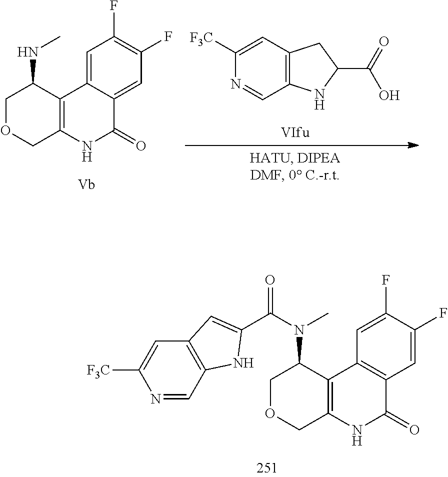

Enantiomerically pure (S)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-c]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-(trifluoromethyl)-1H-pyrrolo[2,3-c]pyridine-2-carboxylic acid (VIfu). LCMS m/z found 479.3 [M+H]⁺; RT=3.61 min (Method D); ¹H NMR (400 MHz, DMSO-d₆) δ 12.75 (s, 1H), 11.77 (s, 1H), 8.95 (s, 1H), 8.21-8.09 (m, 2H), 7.46 (dd, 1H), 7.16 (s, 1H), 5.77 (s, 1H), 4.66 (d, 1H), 4.51 (d, 1H), 4.24 (d, 1H), 4.07 (dd, 1H), 3.14 (s, 3H).

### (S)-2-(3-Bromophenyl)-N-(8,9-difluoro-6-oxo-1,4,5,6-tetrahydro-2H-pyrano[3,4-c]isoquinolin-1-yl)-2,2-difluoro-N-methylacetamide (Compound 252)

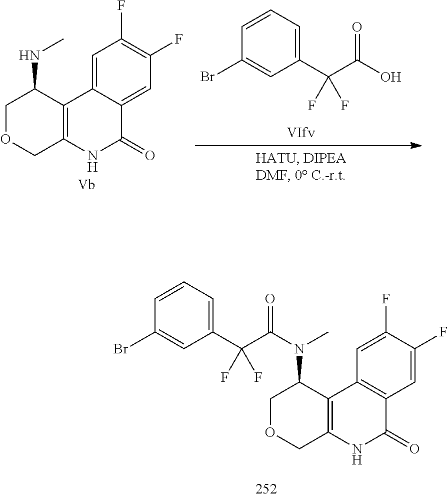 [SMILES: CN[C@@H]1COCc2[nH]c(=O)c3cc(F)c(F)cc3c21 ; O=C(O)C(F)(F)c1cccc(Br)c1 ; CN(C(=O)C(F)(F)c1cccc(Br)c1)[C@@H]1COCc2[nH]c(=O)c3cc(F)c(F)cc3c21]

Enantiomerically pure (S)-2-(3-bromophenyl)-N-(8,9-difluoro-6-oxo-1,4,5,6-tetrahydro-2H-pyrano[3,4-c]isoquinolin-1-yl)-2,2-difluoro-N-methylacetamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 2-(3-bromophenyl)-2,2-difluoro-acetic acid (VIfv). LCMS m/z found 499.2 [M+H]⁺; RT=4.03 min (Method D); ¹H NMR (400 MHz, DMSO-d₆) δ 11.73 (s, 1H), 8.11 (dd, 1H), 7.80 (d, 1H), 7.70 (s, 1H), 7.62 (d, 1H), 7.51 (t, 1H), 7.12 (dd, 1H), 5.55 (s, 1H), 4.55 (d, 1H), 4.43 (d, 1H), 4.13 (d, 1H), 3.98 (dd, 1H), 2.69 (s, 3H).

### (S)-2-(4-Bromophenyl)-N-(8,9-difluoro-6-oxo-1,4,5,6-tetrahydro-2H-pyrano[3,4-c]isoquinolin-1-yl)-2,2-difluoro-N-methylacetamide (Compound 253)

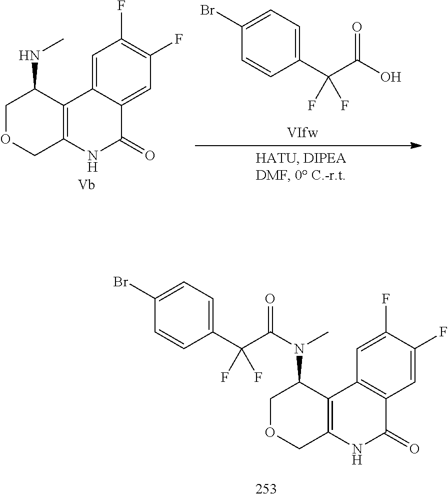

Enantiomerically pure (S)-2-(4-bromophenyl)-N-(8,9-difluoro-6-oxo-1,4,5,6-tetrahydro-2H-pyrano[3,4-c]isoquinolin-1-yl)-2,2-difluoro-N-methylacetamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 2-(4-bromophenyl)-2,2-difluoro-acetic acid (VIfw). LCMS m/z found 499.2 [M+H]⁺; RT=4.02 min (Method D); ¹H NMR (400 MHz, DMSO-d₆) δ 11.72 (s, 1H), 8.10 (dd, 1H), 7.76 (d, 2H), 7.55 (d, 2H), 7.04 (dd, 1H), 5.52 (s, 1H), 4.54 (d, 1H), 4.43 (d, 1H), 4.12 (d, 1H), 3.98 (dd, 1H), 2.67 (s, 3H).

### (S)-N-(8,9-Difluoro-6-oxo-1,4,5,6-tetrahydro-2H-pyrano[3,4-c]isoquinolin-1-yl)-2,2-difluoro-N-methyl-2-phenylacetamide (Compound 512)

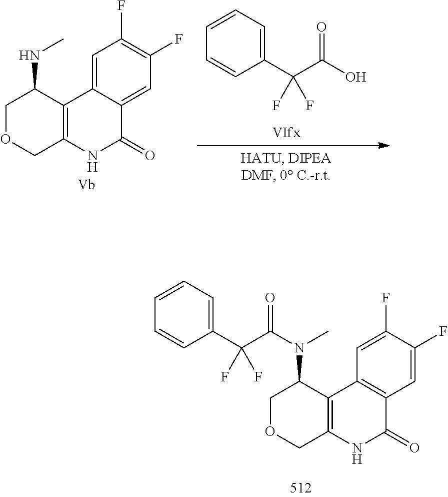

Enantiomerically pure (S)-N-(8,9-difluoro-6-oxo-1,4,5,6-tetrahydro-2H-pyrano[3,4-c]isoquinolin-1-yl)-2,2-difluoro-N-methyl-2-phenylacetamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 2,2-difluoro-2-phenyl-acetic acid (VIfx). LCMS m/z found 421.3 [M+H]⁺; RT=4.17 min (Method D); ¹H NMR (400 MHz, DMSO-d₆) δ 11.68 (s, 1H), 8.09 (t, 1H), 7.62-7.50 (m, 5H), 7.09 (dd, 1H), 5.56 (s, 1H), 4.53 (d, 1H), 4.42 (d, 1H), 4.11 (d, 1H), 3.98 (dd, 1H), 2.68 (s, 3H).

### 5-Bromo-1H-pyrrolo[3,2-b]pyridine-2-carboxylic acid (VIfy)

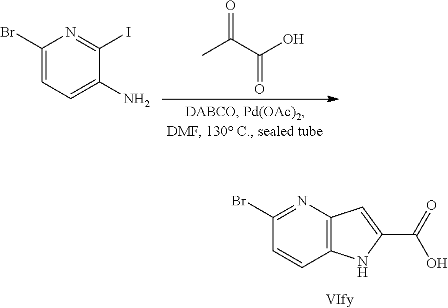

Step i. A dry, large microwave vial with stir bar was charged with 6-bromo-2-iodo-pyridin-3-amine (0.50 g, 1.7 mmol), DABCO (0.56 g, 5.0 mmol), 2-oxopropanoic acid (0.44 g, 5.0 mmol) and dry DMF (10 mL). The mixture was stirred and purged with nitrogen for 15 minutes then Pd(OAc)₂ (376 mg, 1.67 mmol) was added and purging was continued for 10 minutes. The vial was sealed and heated at 130° C. in a reaction block behind a blast shield for 4 hours then cooled to room temperature. Gas pressure buildup was observed and was released carefully via a needle pointed away from the chemist behind the blast shield. The DMF was evaporated under reduced pressure. The residual was taken up in EtOAc (100 mL) and extracted with aqueous NaOH solution (2N, 2×50 mL). The combined aqueous was extracted with EtOAc (50 mL). The aqueous layer was cooled to 0-5° C. and acidified with aq. HCl solution (2N, 105 mL) to pH 3-4. The acidic aqueous mixture was extracted with EtOAc (4×50 mL). The combined organic was washed with sat. aq. brine solution (50 mL), dried over Na₂SO₄, filtered through CELITE® and evaporated under reduced pressure to give 5-bromo-1H-pyrrolo[3,2-b]pyridine-2-carboxylic acid (VIfy) (0.19 g, 47% yield) as a yellow solid. LCMS m/z found 241.1 [M+H]⁺; RT=1.07 min (Method B); ¹H NMR (400 MHz, DMSO-d₆) δ 13.53 (s, 1H), 12.33 (s, 1H), 7.78 (dd, 1H), 7.41 (d, 1H), 7.13 (dd, 1H).

### (S)-5-Bromo-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide (Compound 268)

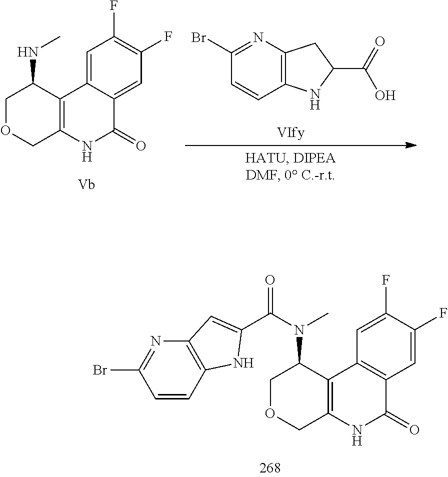

Enantiomerically pure (S)-5-bromo-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-bromo-1H-pyrrolo[3,2-b]pyridine-2-carboxylic acid (VIfy). LCMS m/z found 489.2 [M+H]⁺; RT=3.95 min (Method D); ¹H NMR (400 MHz, DMSO-d₆) δ 12.24 (s, 1H), 11.73 (s, 1H), 8.13 (dd, 1H), 7.80 (d, 1H), 7.45 (dd, 1H), 7.39 (d, 1H), 7.07 (s, 1H), 5.76 (s, 1H), 4.65 (d, 1H), 4.49 (d, 1H), 4.20 (d, 1H), 4.04 (dd, 1H), 3.15 (s, 3H).

### (S)-1-(4-Chlorophenyl)-N-(8,9-difluoro-6-oxo-1,4,5,6-tetrahydro-2H-pyrano[3,4-c]isoquinolin-1-yl)-N-methylazetidine-3-carboxamide (Compound 513)

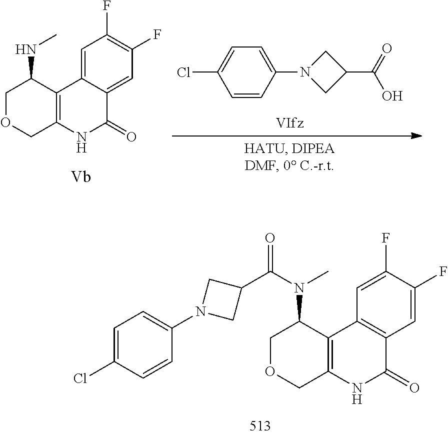 [SMILES: CN[C@@H]1COCc2[nH]c(=O)c3cc(F)c(F)cc3c21 ; O=C(O)C1CN(c2ccc(Cl)cc2)C1 ; CN(C(=O)C1CN(c2ccc(Cl)cc2)C1)[C@@H]1COCc2[nH]c(=O)c3cc(F)c(F)cc3c21]

Enantiomerically pure (S)-1-(4-chlorophenyl)-N-(8,9-difluoro-6-oxo-1,4,5,6-tetrahydro-2H-pyrano[3,4-c]isoquinolin-1-yl)-N-methylazetidine-3-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 1-(4-chlorophenyl)azetidine-3-carboxylic acid (VIfz). LCMS m/z found 460.3 [M+H]⁺; RT=4.13 min (Method D); ¹H NMR (400 MHz, DMSO-d₆) δ 11.69 (s, 1H), 8.11 (dd, 1H), 7.28-7.18 (m, 3H), 6.50-6.44 (m, 2H), 5.56 (s, 1H), 4.57 (d, 1H), 4.42 (d, 1H), 4.13-3.87 (m, 6H), 3.78 (t, 1H), 2.72 (s, 3H).

### N-((S)-8,9-Difluoro-6-oxo-1,4,5,6-tetrahydro-2H-pyrano[3,4-c]isoquinolin-1-yl)-N-methylbicyclo[4.2.0]octa-1(6),2,4-triene-7-carboxamide (Compound 269)

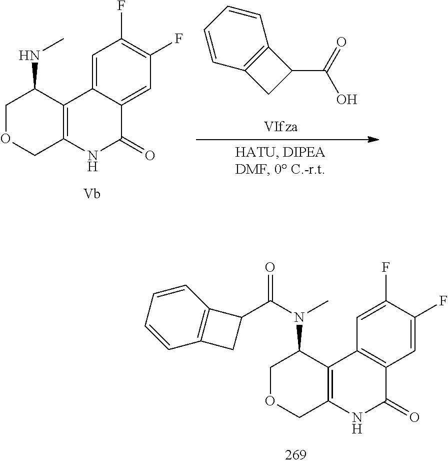

A diastereomeric mixture of N-((S)-8,9-difluoro-6-oxo-1,4,5,6-tetrahydro-2H-pyrano[3,4-c]isoquinolin-1-yl)-N-methylbicyclo[4.2.0]octa-1(6),2,4-triene-7-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and bicyclo[4.2.0]octa-1,3,5-triene-7-carboxylic acid (VIfza). LCMS m/z found 397.3 [M+H]⁺; RT=4.05 min (Method D); ¹H NMR (400 MHz, DMSO-d₆) δ 11.65 (s, 1H), 8.16-8.06 (m, 1H), 7.27-6.88 (m, 5H), 5.57-5.50 (m, 1H), 4.76-4.68 (m, 1H), 4.60 (d, 1H), 4.44 (d, 1H), 4.08-3.90 (m, 2H), 3.59-3.21 (m, 2H), 3.01-2.94 (m, 3H).

### N-[(1S)-8,9-Difluoro-6-oxo-1,2,4,5-tetrahydropyrano[3,4-c]isoquinolin-1-yl]-5-fluoro-N-methyl-6-oxo-1H-pyridine-2-carboxamide (Compound 270)

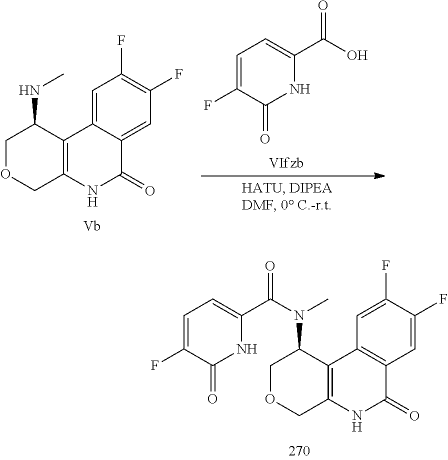

Enantiomerically pure N-[(1S)-8,9-difluoro-6-oxo-1,2,4,5-tetrahydropyrano[3,4-c]isoquinolin-1-yl]-5-fluoro-N-methyl-6-oxo-1H-pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-fluoro-6-oxo-1H-pyridine-2-carboxylic acid (VIfzb) (prepared as described in Bioorganic & Medicinal Chemistry (2009), 17(16), 6106-6122.). LCMS m/z found 406.3 [M+H]⁺; RT=3.30 min (Method D); ¹H NMR (400 MHz, DMSO-d₆) δ 12.75 (s, 1H), 11.72 (s, 1H), 8.13 (dd, 1H), 7.44-7.37 (m, 2H), 6.20 (s, 1H), 5.54 (s, 1H), 4.59 (d, 1H), 4.45 (d, 1H), 4.27 (d, 1H), 3.96 (d, 1H), 2.74 (s, 3H).

### N-((S)-8,9-Difluoro-6-oxo-1,4,5,6-tetrahydro-2H-pyrano[3,4-c]isoquinolin-1-yl)-4-fluoro-N-methylbicyclo[4.2.0]octa-1(6),2,4-triene-7-carboxamide (Compound 279)

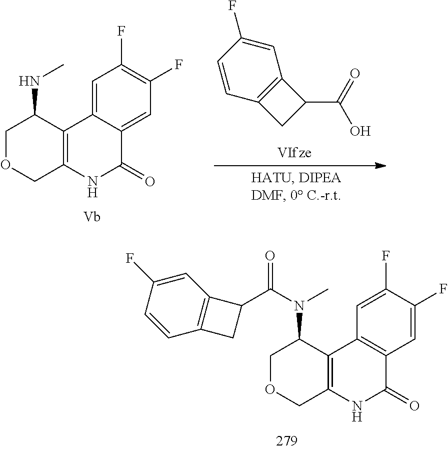

A diastereomeric mixture of N-((S)-8,9-difluoro-6-oxo-1,4,5,6-tetrahydro-2H-pyrano[3,4-c]isoquinolin-1-yl)-4-fluoro-N-methylbicyclo[4.2.0]octa-1(6),2,4-triene-7-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 4-fluorobicyclo[4.2.0]octa-1,3,5-triene-7-carboxylic acid (VIfzc). LCMS m/z found 415.3 [M+H]⁺; RT=4.15 min (Method D); ¹H NMR (400 MHz, DMSO-d₆) δ 11.69 (s, 1H), 8.12 (dd, 1H), 7.23-6.77 (m, 4H), 5.57-5.47 (m, 1H), 4.75-4.65 (m, 1H), 3.52-3.06 (m, 2H), 4.64-4.57 (m, 1H), 4.48-4.41 (m, 1H), 4.09-3.98 (m, 1H), 3.98-3.90 (m, 1H), 2.97-2.90 (m, 3H).

### 1-(Thiophen-3-yl)azetidine-3-carboxylic acid (VIfzd)

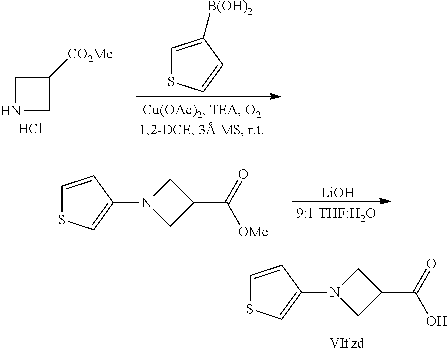

Step i. A mixture of methyl azetidine-3-carboxylate hydrochloride (250.0 mg, 1.7 mmol), 3-thienylboronic acid (253.0 mg, 2.0 mmol), copper(II) acetate (449.0 mg, 2.5 mmol), TEA (1.2 mL, 8.3 mmol) and activated 3 Å MS in dry 1,2-DCE (30 mL) was degassed under vacuum. The reaction was placed under oxygen atmosphere (via balloon) and the dark blue mixture was stirred at room temperature overnight. The reaction mixture was filtered through a plug of CELITE® and the filter cake was rinsed with CH₂Cl₂ (50 mL). The green filtrate was evaporated to a solid and purified via normal phase SiO₂ chromatography (0-50% EtOAc/Hexanes). The desired fractions were collected and evaporated to give methyl 1-(3-thienyl)azetidine-3-carboxylate (57.6 mg, 18% yield) as a tan oil. LCMS m/z found 198.1 [M+H]⁺; RT=2.37 min (Method C); ¹H NMR (400 MHz, CDCl₃) δ 7.19 (dd, 1H), 6.58-6.51 (m, 1H), 5.98-5.93 (m, 1H), 4.07-3.96 (m, 4H), 3.74 (s, 3H), 3.59-3.50 (m, 1H).

Step ii. In a 1-dram vial, to a stirred mixture of methyl 1-(3-thienyl)azetidine-3-carboxylate (57.6 mg, 0.29 mmol) in THF-MeOH (9:1, 1 mL) was added LiOH (8.4 mg, 0.35 mmol). The suspension was stirred at room temperature overnight. The reaction mixture was evaporated to dryness to give 1-(3-thienyl)azetidine-3-carboxylic acid (VIfzd) (53.5 mg, >100% yield), as the lithium salt, as a tan solid. LCMS m/z found 184.1 [M+H]⁺; RT=1.20 min (Method B); ¹H NMR (400 MHz, DMSO-d₆) δ 7.31 (dd, 1H), 6.52 (dd, 1H), 5.90 (dd, 1H), 3.75 (dd, 3H), 3.68 (t, 3H), 2.98 (tt, 1H).

### (S)-N-(8,9-Difluoro-6-oxo-1,4,5,6-tetrahydro-2H-pyrano[3,4-c]isoquinolin-1-yl)-N-methyl-1-(thiophen-3-yl)azetidine-3-carboxamide (Compound 282)

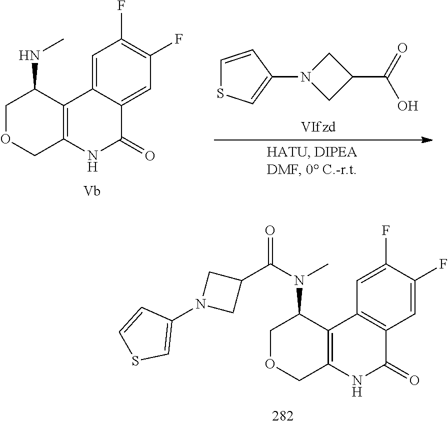 [SMILES: CN[C@@H]1COCc2[nH]c(=O)c3cc(F)c(F)cc3c21 ; O=C(O)C1CN(c2ccsc2)C1 ; CN(C(=O)C1CN(c2ccsc2)C1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-N-methyl-1-(thiophen-3-yl)azetidine-3-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 1-(3-thienyl)azetidine-3-carboxylic acid (VIfzd). LCMS m/z found 432.3 [M+H]⁺; RT=4.05 min (Method D); ¹H NMR (400 MHz, DMSO-d₆) δ 11.68 (s, 1H), 8.11 (dd, 1H), 7.41-7.36 (m, 1H), 7.24 (dd, 1H), 6.63-6.59 (m, 1H), 6.11-6.07 (m, 1H), 5.55 (s, 1H), 4.57 (d, 1H), 4.42 (d, 1H), 4.05-3.84 (m, 6H), 3.72 (t, 1H), 2.70 (s, 3H).

### 1-(4-Bromothiophen-3-yl)azetidine-3-carboxylic acid (VIfze)

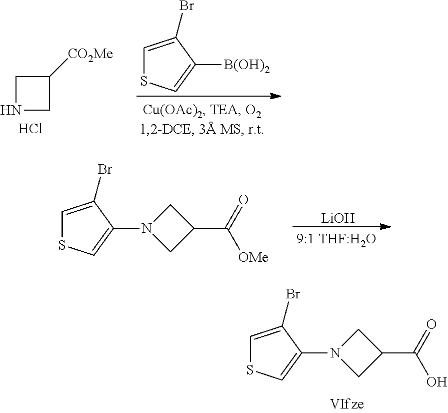 [SMILES: COC(=O)C1CNC1 ; OB(O)c1cscc1Br ; Cl ; COC(=O)C1CN(c2cscc2Br)C1 ; "LiOH" ; O=C(O)C1CN(c2cscc2Br)C1]

Step i. A mixture of methyl azetidine-3-carboxylate hydrochloride (1.00 g, 6.6 mmol), (4-bromo-3-thienyl)boronic acid (1.64 g, 7.92 mmol), copper(II) acetate (1.80 g, 9.90 mmol), TEA (4.60 mL, 33.0 mmol) and 3 Å MS in dry 1,2-DCE (30 mL) was degassed under vacuum. The reaction was placed under oxygen atmosphere (via balloon) and the dark blue mixture was stirred at room temperature over the weekend. The blue-green suspension was filtered through a plug of CELITE® and the filter cake was rinsed with CH₂Cl₂. The green filtrate was evaporated and purified via normal phase SiO₂ chromatography (0-50% EtOAc/Hexanes). The desired fractions were collected and evaporated under reduced pressure to give methyl 1-(4-bromo-3-thienyl)azetidine-3-carboxylate (66.8 mg, 4% yield) as a tan oil. LCMS m/z found 276.1 [M+H]⁺; RT=2.93 min (Method C); ¹H NMR (400 MHz, CDCl₃) δ 7.18-7.12 (m, 1H), 6.13-6.05 (m, 1H), 4.23-4.17 (m, 2H), 4.07-4.01 (m, 2H), 3.74 (s, 3H), 3.58-3.48 (m, 1H).

Step ii. In a 1-dram vial, to a stirred mixture of methyl 1-(4-bromo-3-thienyl)azetidine-3-carboxylate (66.8 mg, 0.24 mmol) in THF-MeOH (9:1, 1 mL) was added LiOH (7.0 mg, 0.3 mmol). The suspension was stirred at room temperature overnight. The reaction mixture was evaporated to dryness to give 1-(4-bromo-3-thienyl)azetidine-3-carboxylic acid (VIfze) (63.4 mg, >100% yield) as the lithium salt, as an off-white solid. LCMS m/z found 262.1 [M+H]⁺; RT=1.20 min (Method B); ¹H NMR (400 MHz, DMSO-d₆) δ 7.49 (d, 1H), 6.23 (d, 1H), 3.89 (dd, 3H), 3.77 (t, 3H), 2.93 (tt, 1H).

### (S)-1-(4-Bromothiophen-3-yl)-N-(8,9-difluoro-6-oxo-1,4,5,6-tetrahydro-2H-pyrano[3,4-c]isoquinolin-1-yl)-N-methylazetidine-3-carboxamide (Compound 284)

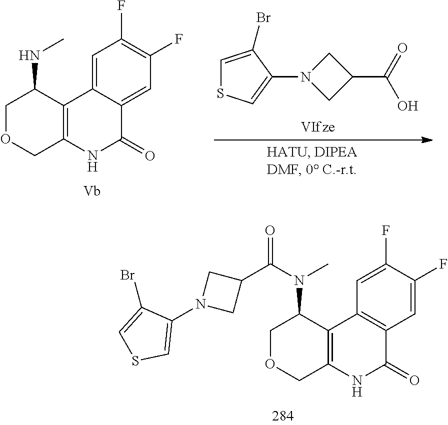 [SMILES: CN[C@@H]1COCc2[nH]c(=O)c3cc(F)c(F)cc3c21 ; O=C(O)C1CN(c2cscc2Br)C1 ; CN(C(=O)C1CN(c2cscc2Br)C1)[C@@H]1COCc2[nH]c(=O)c3cc(F)c(F)cc3c21]

Enantiomerically pure (S)-1-(4-bromothiophen-3-yl)-N-(8,9-difluoro-6-oxo-1,4,5,6-tetrahydro-2H-pyrano[3,4-c]isoquinolin-1-yl)-N-methylazetidine-3-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 1-(4-bromo-3-thienyl)azetidine-3-carboxylic acid (VIfze). LCMS m/z found 510.2 [M+H]⁺; RT=4.42 min (Method D); ¹H NMR (400 MHz, DMSO-d₆) δ 11.69 (s, 1H), 8.15-8.07 (m, 1H), 7.58 (dd, 1H), 7.25 (dd, 1H), 6.45 (dd, 1H), 5.56 (s, 1H), 4.57 (d, 1H), 4.42 (d, 1H), 4.18-4.08 (m, 2H), 4.07-3.97 (m, 2H), 3.96-3.79 (m, 3H), 2.71 (s, 3H).

### (S)-N-(8,9-Difluoro-6-oxo-1,4,5,6-tetrahydro-2H-pyrano[3,4-c]isoquinolin-1-yl)-N-methyl-1H-pyrrolo[2,3-c]pyridine-2-carboxamide (Compound 307)

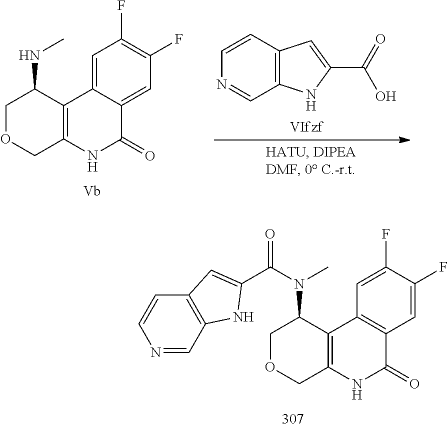

Enantiomerically pure (S)-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 1H-pyrrolo[2,3-c]pyridine-2-carboxylic acid (VIfzf). LCMS m/z found 411.2 [M+H]⁺; RT=1.24 min (Method D); ¹H NMR (400 MHz, DMSO-d₆) δ 12.23 (s, 1H), 11.76 (s, 1H), 8.84 (s, 1H), 8.18-8.09 (m, 2H), 7.59 (d, 1H), 7.44 (dd, 1H), 6.98 (s, 1H), 5.76 (s, 1H), 4.65 (d, 1H), 4.49 (d, 1H), 4.20 (d, 1H), 4.08-4.02 (m, 1H), 3.14 (s, 3H).

### (S)-N-(8,9-Difluoro-6-oxo-1,4,5,6-tetrahydro-2H-pyrano[3,4-c]isoquinolin-1-yl)-N-methyl-1H-pyrrolo[3,2-c]pyridine-2-carboxamide (Compound 308)

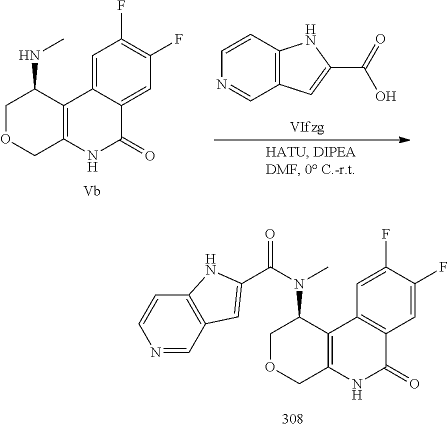

Enantiomerically pure (S)-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 1H-pyrrolo[3,2-c]pyridine-2-carboxylic acid (VIfzg). LCMS m/z found 411.2 [M+H]⁺; RT=1.22 min (Method D); ¹H NMR (400 MHz, DMSO-d₆) δ 12.14 (s, 1H), 11.76 (s, 1H), 8.91 (s, 1H), 8.24 (d, 1H), 8.13 (dd, 1H), 7.48-7.38 (m, 2H), 7.11 (s, 1H), 5.76 (s, 1H), 4.65 (d, 1H), 4.49 (d, 1H), 4.19 (d, 1H), 4.05 (dd, 1H), 3.17 (s, 3H).

### (S)-6-Chloro-N-(8,9-difluoro-6-oxo-1,4,5,6-tetrahydro-2H-pyrano[3,4-c]isoquinolin-1-yl)-N-methyl-1¹H-pyrrolo[3,2-c]pyridine-2-carboxamide (Compound 309)

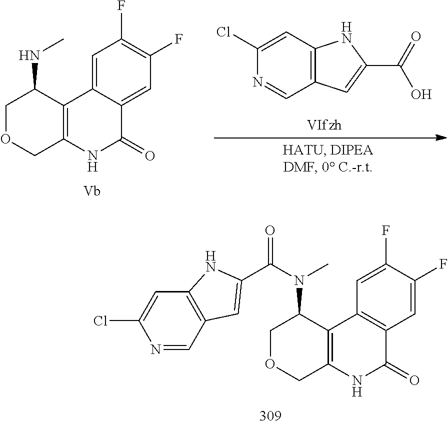 [SMILES: CN[C@@H]1COCc2[nH]c(=O)c3cc(F)c(F)cc3c21 ; O=C(O)c1cc2cnc(Cl)cc2[nH]1 ; CN(C(=O)c1cc2cnc(Cl)cc2[nH]1)[C@@H]1COCc2[nH]c(=O)c3cc(F)c(F)cc3c21]

Enantiomerically pure (S)-6-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 6-chloro-1H-pyrrolo[3,2-c]pyridine-2-carboxylic acid (VIfzh). LCMS m/z found 445.2 [M+H]⁺; RT=1.87 min (Method D); ¹H NMR (400 MHz, DMSO-d₆) δ 12.29 (s, 1H), 11.76 (s, 1H), 8.73 (s, 1H), 8.13 (dd, 1H), 7.48-7.40 (m, 2H), 7.16 (s, 1H), 5.75 (s, 1H), 4.65 (d, 1H), 4.49 (d, 1H), 4.19 (d, 1H), 4.08-4.00 (m, 1H), 3.16 (s, 3H).

### 5-Fluoro-1H-pyrrolo[2,3-c]pyridine-2-carboxylic acid (VIfzi)

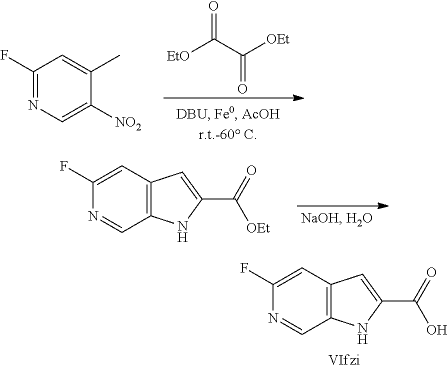

Step i. To a solution of 2-fluoro-4-methyl-5-nitro-pyridine (1.0 g, 6.4 mmol) and diethyl oxalate, (2.2 mL, 16.0 mmol) at room temperature was added dropwise DBU (1.1 mL, 7.1 mmol). The mixture was stirred at room temperature for 4 hours. The mixture was diluted with EtOAc (50 mL) then a mixture of AcOH (1 mL) and water (20 mL). The layers were separated and the aqueous was extracted with EtOAc (2×20 mL). The combined organic layer was washed with water (20 mL) and sat. NaCl solution (20 mL). The organic was dried over Na₂SO₄, filtered and evaporated under reduced pressure. The recovered oil was purified via normal phase SiO₂ chromatography (0%-40% EtOAc/Hexane). The desired fractions were collected and evaporated under reduced pressure to give ethyl 3-(2-fluoro-5-nitropyridin-4-yl)-2-oxopropanoate (0.42 g) as a cherry-red solid. LCMS m/z found 255.1 [M+H]⁺; RT=2.75 min (Method C); ¹H NMR (400 MHz, DMSO-d₆) δ 8.91 (s, 1H), 7.89 (s, 1H), 6.72 (d, 1H), 4.71 (s, 1H), 4.31 (q, 2H), 1.30 (t, 3H).

Step ii. The cherry-red solid (0.42 g) from above was dissolved in AcOH (10 mL) and warmed to 60° C. under nitrogen then powdered iron (0.72 g, 12.8 mmol) was added. The mixture was heated at 70° C. under nitrogen. After 2 hours, the mixture was cooled to room temperature then diluted with water (25 mL). The resulting solid was filtered, rinsed with water and partially dried by suction. The solid was suspended in MeOH (20 mL), sonicated for 1 minute then filtered through plug of CELITE®. The filtrate was evaporated under reduced pressure to give ethyl 5-fluoro-1H-pyrrolo[2,3-c]pyridine-2-carboxylate (0.31 g, 22% yield) as a tan solid. LCMS m/z found 209.1 [M+H]⁺; RT=2.57 min (Method C); 1H NMR (400 MHz, DMSO-d₆) δ 12.51 (s, 1H), 8.48-8.43 (m, 1H), 7.35-7.30 (m, 1H), 7.19-7.15 (m, 1H), 4.38 (q, 2H), 1.35 (t, 4H).

Step iii. A suspension of ethyl 5-fluoro-1H-pyrrolo[2,3-c]pyridine-2-carboxylate (150 mg, 0.72 mmol) in aq. NaOH solution (1M, 3 mL) was stirred at room temperature for 2 hours, a solution resulted. The reaction mixture was cooled to 5° C. and acidified with aq. HCl solution (1M, ˜4 mL). The mixture was stirred for 10 minutes then filtered, rinsed with water and partially dried by suction. The solid was dried under high vacuum overnight to give 5-fluoro-1H-pyrrolo[2,3-c]pyridine-2-carboxylic acid (VIfzi) (129.6 mg, 99%) as an off-white solid. LCMS m/z found 181.1 [M+H]⁺; RT=1.88 min (Method C); ¹H NMR (400 MHz, DMSO-d₆) δ 13.63 (s, 1H), 12.38 (s, 1H), 8.44-8.40 (m, 1H), 7.33-7.29 (m, 1H), 7.12-7.08 (m, 1H).

### (S)-N-(8,9-Difluoro-6-oxo-1,4,5,6-tetrahydro-2H-pyrano[3,4-c]isoquinolin-1-yl)-5-fluoro-N-methyl-1H-pyrrolo[2,3-c]pyridine-2-carboxamide (Compound 310)

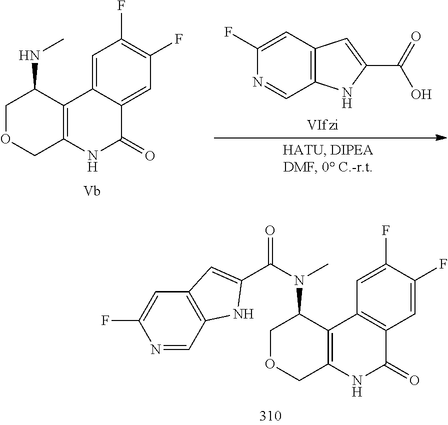 [SMILES: CN[C@@H]1COCc2[nH]c(=O)c3cc(F)c(F)cc3c21 ; O=C(O)c1cc2cc(F)ncc2[nH]1 ; CN(C(=O)c1cc2cc(F)ncc2[nH]1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-5-fluoro-N-methyl-1H-pyrrolo[2,3-c]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-fluoro-1H-pyrrolo[2,3-c]pyridine-2-carboxylic acid (VIfzi). LCMS m/z found 429.2 [M+H]⁺; RT=2.03 min (Method D); ¹H NMR (400 MHz, DMSO-d₆) δ 12.32 (s, 1H), 11.75 (s, 1H), 8.43 (s, 1H), 8.14 (dd, 1H), 7.43 (dd, 1H), 7.26 (s, 1H), 6.96 (s, 1H), 5.74 (s, 1H), 4.64 (d, 1H), 4.49 (d, 1H), 4.21 (d, 1H), 4.05 (dd, 1H), 3.11 (s, 3H).

### 4-Bromo-1H-pyrrolo[2,3-c]pyridine-2-carboxylic acid (VIfzj)

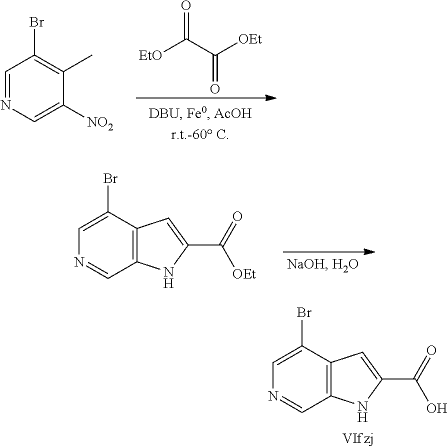

Step i. To a solution of 3-bromo-4-methyl-5-nitro-pyridine (1.0 g, 4.6 mmol) and diethyl oxalate, (2.9 mL, 21 mmol) at room temperature was added dropwise DBU (0.76 mL, 5.1 mmol). The reaction mixture was stirred at room temperature under nitrogen overnight. The reaction mixture was evaporated at 70° C. water bath under reduced pressure. The residue was dissolved in AcOH (10 mL) and warmed to 60° C. then powdered iron (0.51 g, 9.2 mmol) was added. The reaction mixture was stirred vigorously under nitrogen and heated at 70° C. After 2 hours, the reaction mixture was cooled to room temperature, diluted with water 30 mL and the resulting precipitate was filtered and rinsed with water and partially dried by suction. The solid was suspended in MeOH, sonicated for 1 minute then filtered through a plug of CELITE®, the filtercake was rinsed with MeOH. The filtrate was evaporated to give ethyl 4-bromo-1H-pyrrolo[2,3-c]pyridine-2-carboxylate (0.60 g, 46% yield) as a dark, red-brown solid. LCMS m/z found 269.0 [M+H]⁺; RT=2.23 min (Method C); ¹H NMR (400 MHz, DMSO-d₆) δ 12.88 (s, 1H), 8.82 (s, 1H), 8.34 (s, 1H), 7.07 (d, 1H), 4.39 (q, 2H), 1.36 (t, 3H).

Step ii. A suspension of ethyl 4-bromo-1H-pyrrolo[2,3-c]pyridine-2-carboxylate (150 mg, 0.56 mmol) in aq. NaOH solution (1M, 3 mL) was stirred at room temperature overnight. Additional aq. NaOH solution (1M, 1 mL) and MeOH (1 mL) was added to the suspension and the mixture stirred at room temperature for 4 hours. The reaction mixture was cooled to 5° C. and acidified with aq. HCl solution (1M, ˜5 mL). The mixture was stirred for 10 minutes then extracted with EtOAc (50 mL). The layers were separated. The organic layer was dried over Na₂SO₄, filtered and evaporated then subjected to high vacuum for 1 hour to give 4-bromo-1H-pyrrolo[2,3-c]pyridine-2-carboxylic acid (VIfzj) (134 mg, 100% yield) as a light-brown solid. LCMS m/z found 241.0 [M+H]⁺; RT=1.01 min (Method C); ¹H NMR (400 MHz, DMSO-d₆) δ 13.74 (s, 1H), 12.77 (s, 1H), 8.80 (s, 1H), 8.32 (s, 1H), 7.02 (d, 1H).

### (S)-4-Bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide (Compound 312)

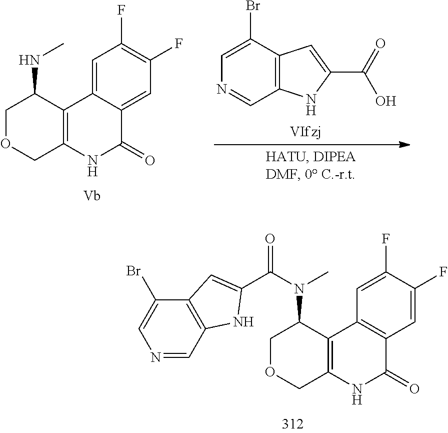

Enantiomerically pure (S)-4-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 4-bromo-1H-pyrrolo[2,3-c]pyridine-2-carboxylic acid (VIfzj). LCMS m/z found 489.1 [M+H]⁺; RT=1.53 min (Method D); ¹H NMR (400 MHz, DMSO-d₆) δ 12.66 (s, 1H), 11.76 (s, 1H), 8.81 (s, 1H), 8.29 (s, 1H), 8.14 (dd, 1H), 7.45 (dd, 1H), 6.89 (s, 1H), 5.74 (s, 1H), 4.64 (d, 1H), 4.49 (d, 1H), 4.23 (d, 1H), 4.08-4.01 (m, 1H), 3.13 (s, 3H).

### (S)-N-(8,9-Difluoro-6-oxo-1,4,5,6-tetrahydro-2H-pyrano[3,4-c]isoquinolin-1-yl)-2-(difluoromethyl)-N-methylisonicotinamide (Compound 313)

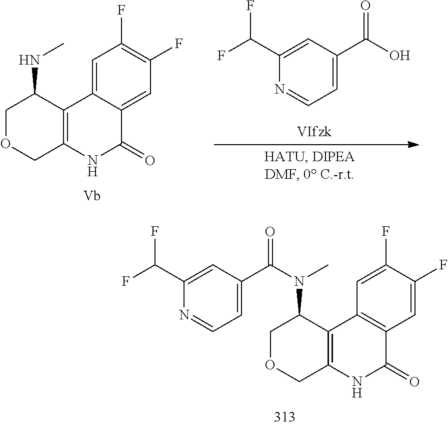

Enantiomerically pure (S)-N-(8,9-difluoro-6-oxo-1,4,5,6-tetrahydro-2H-pyrano[3,4-c]isoquinolin-1-yl)-2-(difluoromethyl)-N-methylisonicotinamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 2-(difluoromethyl)pyridine-4-carboxylic acid (VIfzk). LCMS m/z found 422.1 [M+H]⁺; RT=1.97 min (Method D); ¹H NMR (400 MHz, DMSO-d₆) δ 11.71 (s, 1H), 8.79 (d, 1H), 8.18-8.11 (m, 1H), 7.75 (s, 1H), 7.60 (d, 1H), 7.48 (dd, 1H), 7.02 (t, 1H), 5.68 (s, 1H), 4.59 (d, 1H), 4.47 (d, 1H), 4.36 (d, 1H), 4.07-3.96 (m, 1H), 2.68 (s, 3H).

### 7-Bromofuro[3,2-c]pyridine-2-carboxylic acid (VIfzl)

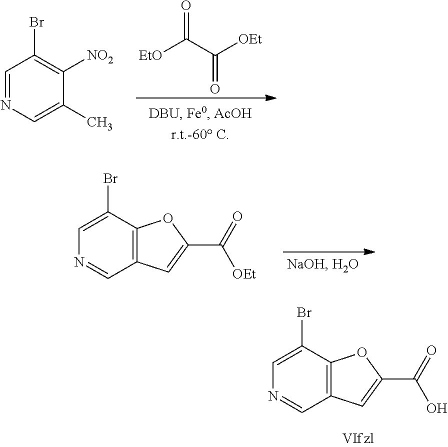

Step i. To a solution of 3-bromo-5-methyl-4-nitro-pyridine (0.50 g, 2.3 mmol) and diethyl oxalate (1.44 mL, 10.6 mmol) at room temperature was added dropwise DBU (0.38 mL, 2.5 mmol). The reaction mixture was stirred at room temperature under nitrogen for 48 hours. The mixture was heated at 100° C. for 1 hour. The reaction mixture was cooled to room temperature. The residue was dissolved in AcOH (10 mL) and warmed to 60° C. then powdered iron (0.26 g, 4.6 mmol) was added. The reaction mixture was stirred vigorously under nitrogen and heated at 70° C. for 2 hours. The reaction mixture was cooled to room temperature, diluted with water 30 mL and the resulting precipitate was filtered, rinsed with water and partially dried by suction. The crude red-brown solid contained no desired product mass; however, the acidic aqueous layer contained desired mass. The aqueous was evaporated under reduced pressure to give a dark-brown viscous resin. The resin was partitioned between water (50 mL) and EtOAc (50 ml) then carefully made basic with aq. K₂CO₃ solution (1 M) to pH 9. The aqueous was extracted with EtOAc (2×50 mL). The combined organic was washed with water (2×50 mL) then sat. NaCl solution (15 mL). The organic layer was dried over Na₂SO₄, filtered and evaporated under reduced pressure. The residue was purified via normal phase SiO₂ chromatography (0-100% EtOAc/Hexane). The desired fractions were collected and evaporated to give ethyl 7-bromofuro[3,2-c]pyridine-2-carboxylate (26.7 mg, 4% yield) as a pale-yellow solid. LCMS m/z found 269.9 [M+H]⁺; RT=2.72 min (Method C); ¹H NMR (400 MHz, CDCl₃) δ 8.94 (s, 1H), 8.71 (s, 1H), 7.63 (s, 1H), 4.48 (q, 2H), 1.45 (t, 3H).

Step ii. A 1-dram vial with stir bar was charged with ethyl 7-bromofuro[3,2-c]pyridine-2-carboxylate (26.7 mg, 0.10 mmol), MeOH (0.5 mL) and NaOH solution (1 M, 0.5 mL, 5 eq.) and the mixture was stirred vigorously. After 4 hours, the mixture pH was adjusted with HCl solution (1M, 0.6 mL). The resulting precipitate was filtered and rinsed with water. The solid was azeotroped with MeOH (2×10 mL) to give 7-bromofuro[3,2-c]pyridine-2-carboxylic acid (VIfzl) (16.7 mg, 71% yield) as a white solid. The recovered material was used without further purification. LCMS m/z found 243.0 [M+H]⁺; RT=1.02 min (Method C); ¹H NMR (400 MHz, DMSO-d₆) δ 9.05 (s, 1H), 8.77 (s, 1H), 7.89 (s, 1H).

### (S)-7-Bromo-N-(8,9-difluoro-6-oxo-1,4,5,6-tetrahydro-2H-pyrano[3,4-c]isoquinolin-1-yl)-N-methylfuro[3,2-c]pyridine-2-carboxamide (Compound 356)

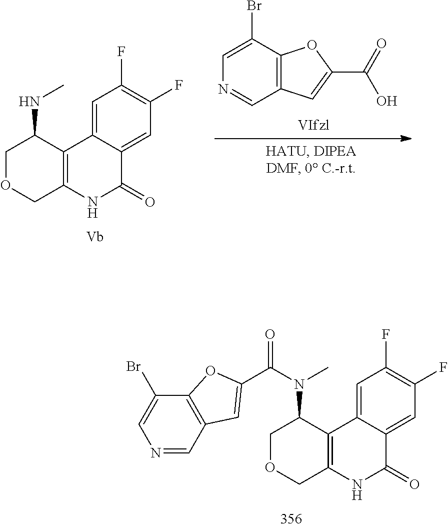

Enantiomerically pure (S)-7-bromo-N-(8,9-difluoro-6-oxo-1,4,5,6-tetrahydro-2H-pyrano[3,4-c]isoquinolin-1-yl)-N-methylfuro[3,2-c]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 7-bromofuro[3,2-c]pyridine-2-carboxylic acid (VIfzl). LCMS m/z found 490.0 [M+H]⁺; RT=1.99 min (Method D); ¹H NMR (400 MHz, DMSO-d₆) δ 11.76 (s, 1H), 9.01 (s, 1H), 8.74 (s, 1H), 8.23-8.09 (m, 1H), 7.79 (s, 1H), 7.40 (dd, 1H), 5.67 (s, 1H), 4.64 (d, 1H), 4.49 (d, 1H), 4.23 (d, 1H), 4.08-3.99 (m, 1H), 3.10 (s, 3H).

### 3-Chloro-1-(difluoromethyl)-2H-indazole-6-carboxylic acid (VIfzm), 3-chloro-2-(difluoromethyl)-2H-indazole-6-carboxylic acid (VIfzn), 2-(difluoromethyl)-3-methoxy-2H-indazole-6-carboxylic acid (VIfzo) and 3-methoxy-1H-indazole-6-carboxylic acid (VIfzp)

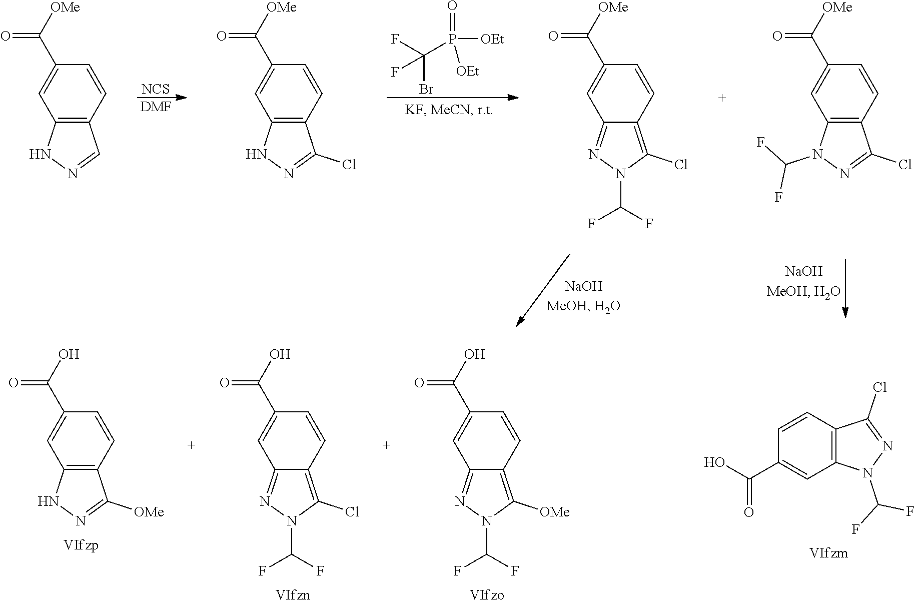

Step i. To a stirred solution of methyl 1H-indazole-6-carboxylate (1.0 g, 5.7 mmol) in dry DMF (10 mL) was added N-Chlorosuccinimide (0.83 g, 6.2 mmol) and the mixture was stirred at room temperature overnight. Water (50 mL) was added to the mixture with vigorous stirring to yield a thick, waxy solid. The solid was filtered and rinsed with water. The waxy solid was dissolved in EtOAc (50 mL) and the residual aqueous layer was separated. The organic layer was washed with sat. aq. Na₂S₂O₃ solution (10 mL), water (2×10 mL) and sat. aq. NaCl solution (10 mL). The organic layer was dried over Na₂SO₄, filtered and evaporated under reduced pressure to give methyl 3-chloro-1H-indazole-6-carboxylate (1.16 g, 97% yield) as a white solid. LCMS m/z found 211.0 [M+H]⁺; RT=2.66 min (Method C); ¹H NMR (400 MHz, CDCl₃) δ 10.12 (s, 1H), 8.23 (s, 1H), 7.92-7.89 (m, 1H), 7.76 (d, 1H), 3.98 (s, 3H).

Step ii. To a dry scintillation vial with stir bar and cap with rubber septa was added methyl 3-chloro-1H-indazole-6-carboxylate (0.10 g, 0.47 mmol) and anhydrous potassium fluoride (60 mg, 0.95 mmol) and the vial was sealed and held under nitrogen atmosphere. Dry acetonitrile (5 mL) was added via syringe. The mixture was stirred vigorously for 5 minutes then diethyl(bromodifluoromethyl)phosphonate (80 μL, 0.47 mmol) was added via syringe. The mixture was stirred at room temperature, under nitrogen, overnight. Additional potassium fluoride (60 mg, 0.95 mmol) and diethyl (bromodifluoromethyl)phosphonate (80 μL, 0.47 mmol) were added and the suspension was stirred vigorously for 24 hours. The mixture was filtered through a plug of CELITE® and rinsed with EtOAc. The filtrate was evaporated under reduced pressure. The residue was purified by flash SiO₂ chromatography (0-40% EtOAc/Hexanes). The desired fractions were collected and evaporated to give methyl 3-chloro-1-(difluoromethyl)-1H-indazole-6-carboxylate (44.1 mg, 36% yield) as an-off white solid, first eluted peak. LCMS m/z found 261.1 [M+H]⁺; RT=3.18 min (Method C); ¹H NMR (400 MHz, CDCl₃) δ 8.46 (t, 1H), 8.05 (dd, 1H), 7.79 (dd, 1H), 7.40 (d, 1H), 4.00 (s, 3H).

Methyl 3-chloro-2-(difluoromethyl)-2H-indazole-6-carboxylate (44.1 mg, 36% yield) as an off-white solid, second eluted peak. LCMS m/z found 261.0 [M+H]⁺; RT=2.97 min (Method C); 1H NMR (400 MHz, CDCl₃) δ 8.46 (t, 1H), 7.79 (dd, 1H), 7.65 (dd, 1H), 7.54 (t, 1H), 3.97 (s, 3H).

Step iii. A 1-dram vial with stir bar was charged with methyl 3-chloro-1-(difluoromethyl)-1H-indazole-6-carboxylate (44 mg, 0.17 mmol), MeOH (0.5 mL) and aq. NaOH solution (1M, 0.9 mL, 5 eq.). The reaction mixture was stirred vigorously overnight at room temperature. The reaction mixture was cooled to 0-5° C. and aq. HCl solution (1N, ˜1 mL) was added to adjust pH to ˜5-6. The resulting suspension was stirred, filtered, rinsed with water and dried by suction to give 3-chloro-1-(difluoromethyl)-1H-indazole-6-carboxylic acid (VIfzm) (39.9 mg, 95% yield) as a white solid. LCMS m/z found 247.0 [M+H]⁺; RT=2.74 min (Method C); ¹H NMR (400 MHz, DMSO-d₆) δ 13.61 (s, 1H), 8.52 (d, 1H), 8.36 (t, 1H), 7.99 (dd, 1H), 7.94 (dd, 1H).

Step iv. 3-Chloro-2-(difluoromethyl)-2H-indazole-6-carboxylate (89 mg, 0.34 mmol) was reacted in an analogous manner as described above, without purification, to give a crude mixture of 2-(difluoromethyl)-3-methoxy-2H-indazole-6-carboxylic acid (VIfzo), 3-methoxy-1H-indazole-6-carboxylic acid (VIfzp) and desired 3-chloro-1-(difluoromethyl)-2H-indazole-6-carboxylic acid (VIfzn) (81 mg, 75% yield) as a white solid. LCMS m/z found 247.0 [M+H]⁺; RT=2.53 min (Method C); ¹H NMR (400 MHz, DMSO-d₆) δ 13.38 (s, 1H), 8.51-8.33 (m, 1H), 8.27 (t, 1H), 7.80 (dd, 1H), 7.71 (dd, 1H).

### (S)-3-Chloro-N-(8,9-difluoro-6-oxo-1,4,5,6-tetrahydro-2H-pyrano[3,4-c]isoquinolin-1-yl)-1-(difluoromethyl)-N-methyl-1H-indazole-6-carboxamide (Compound 357)

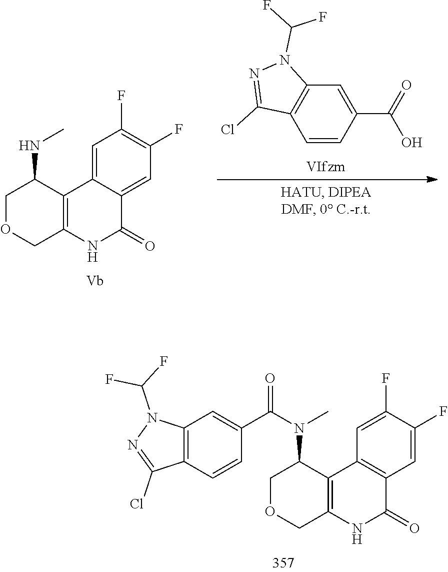 [SMILES: CN[C@@H]1COCc2[nH]c(=O)c3cc(F)c(F)cc3c21 ; O=C(O)c1ccc2c(Cl)nn(C(F)F)c2c1 ; CN(C(=O)c1ccc2c(Cl)nn(C(F)F)c2c1)[C@@H]1COCc2[nH]c(=O)c3cc(F)c(F)cc3c21]

Enantiomerically pure (S)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-6-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 3-chloro-1-(difluoromethyl)-1H-indazole-6-carboxylic acid (VIfzm). LCMS m/z found 495.1 [M+H]⁺; RT=2.70 min (Method D); ¹H NMR (400 MHz, DMSO-d₆) δ 11.73 (s, 1H), 8.43-8.11 (m, 2H), 8.02 (s, 1H), 7.96-7.90 (m, 1H), 7.55 (dd, 1H), 7.46-7.40 (m, 1H), 5.73 (s, 1H), 4.60 (d, 1H), 4.48 (d, 1H), 4.34 (d, 1H), 4.05 (dd, 1H), 2.74-2.68 (m, 3H).

### (S)-3-Chloro-N-(8,9-difluoro-6-oxo-1,4,5,6-tetrahydro-2H-pyrano[3,4-c]isoquinolin-1-yl)-2-(difluoromethyl)-N-methyl-2H-indazole-6-carboxamide (Compound 358)

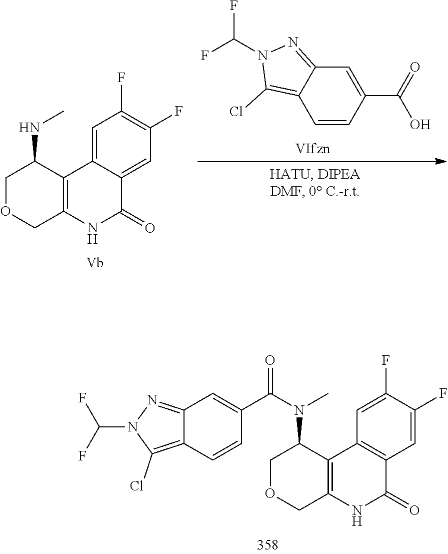 [SMILES: CN[C@@H]1COCc2[nH]c(=O)c3cc(F)c(F)cc3c21 ; O=C(O)c1ccc2c(Cl)n(C(F)F)nc2c1 ; CN(C(=O)c1ccc2c(Cl)n(C(F)F)nc2c1)[C@@H]1COCc2[nH]c(=O)c3cc(F)c(F)cc3c21]

Enantiomerically pure (S)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-6-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and crude 3-chloro-2-(difluoromethyl)-2H-indazole-6-carboxylic acid (VIfzn). LCMS m/z found 495.1 [M+H]⁺; RT=2.51 min (Method D); ¹H NMR (400 MHz, DMSO-d₆) δ 11.72 (s, 1H), 8.46-8.11 (m, 2H), 7.83-7.78 (m, 2H), 7.53 (dd, 1H), 7.23-7.17 (m, 1H), 5.71 (s, 1H), 4.60 (d, 1H), 4.47 (d, 1H), 4.33 (d, 1H), 4.05 (dd, 1H), 2.74 (d, 3H).

### 3-Chloro-1-(difluoromethyl)indazole-5-carboxylic acid (VIfzq) and 3-chloro-2-(difluoromethyl)indazole-5-carboxylic acid (VIfzr)

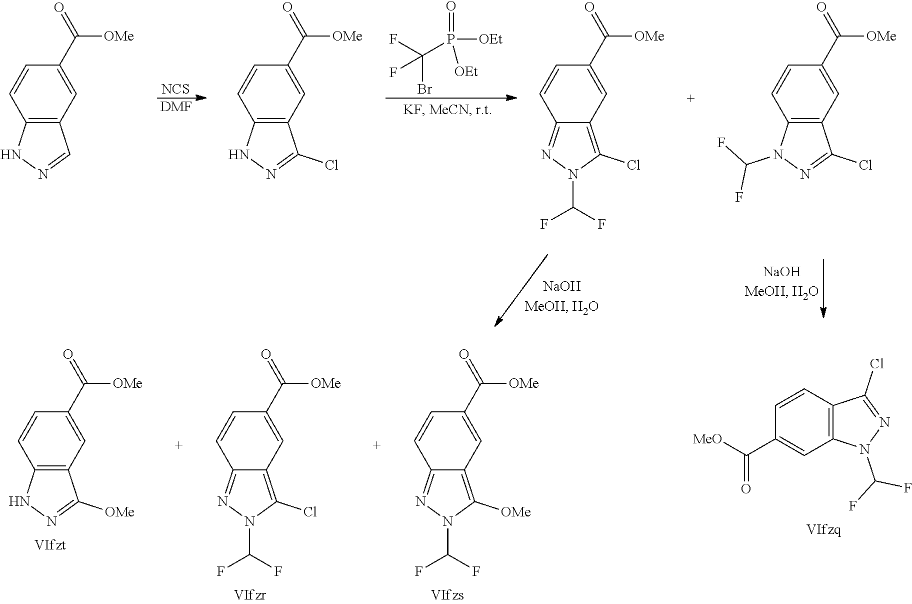

Step i. To a stirred solution of methyl 1H-indazole-5-carboxylate (1.0 g, 5.7 mmol) in dry DMF (10 mL) was added N-Chlorosuccinimide (0.83 g, 6.2 mmol). The mixture was stirred at room temperature overnight. Water (50 mL) was added to the mixture with vigorous stirring to yield a thick, waxy solid. The solid was filtered and rinsed with water. The waxy solid was dissolved in EtOAc (50 mL) and the residual aqueous layer was separated. The organic layer was washed with sat. aq. Na₂S₂O₃ solution (10 mL), water (2×10 mL) and sat. aq. NaCl solution (10 mL). The organic layer was dried over Na₂SO₄, filtered and evaporated to give methyl 3-chloro-1H-indazole-5-carboxylate (1.10 g, 92% yield) as an off-white solid. LCMS m/z found 211.1 [M+H]⁺; RT=2.61 min (Method C); ¹H NMR (400 MHz, CDCl₃) δ 10.21 (s, 1H), 8.49 (s, 1H), 8.16-8.12 (m, 1H), 7.52-7.48 (m, 1H), 3.97 (s, 3H).

Step ii. To a dry scintillation vial with stir bar and cap with rubber septa was added methyl 3-chloro-1H-indazole-5-carboxylate (0.25 g, 1.2 mmol) and anhydrous potassium fluoride (0.28 g, 4.7 mmol) and the vial was sealed and held under nitrogen atmosphere. Dry acetonitrile (5 mL) was added via syringe. The mixture was stirred vigorously for 5 minutes then diethyl bromodifluoromethyl)phosphonate (0.32 mL, 1.8 mmol) was added via syringe. The mixture was stirred at room temperature, under nitrogen, for 5 hours. The mixture was filtered through a plug of CELITE® and rinsed with EtOAc. The filtrate was evaporated and the residue was purified via normal phase SiO₂ chromatography (0-30% EtOAc/Hexanes). The desired fractions were collected and evaporated to give methyl 3-chloro-1-(difluoromethyl)-1H-indazole-5-carboxylate (127.3 mg, 44% yield) as an-off white solid, first eluted peak. LCMS m/z found 261.0 [M+H]⁺; RT=3.15 min (Method C); ¹H NMR (400 MHz, CDCl₃) δ 8.50-8.48 (m, 1H), 8.25 (dd, 1H), 7.81-7.76 (m, 1H), 7.38 (t, 1H), 3.99 (s, 3H). 3-Chloro-2-(difluoromethyl)-2H-indazole-5-carboxylate (110.1 mg, 36% yield) as an off-white solid, second eluted peak. LCMS m/z found 261.0 [M+H]⁺; RT=2.94 min (Method C); ¹H NMR (400 MHz, CDCl₃) δ 8.47-8.43 (m, 1H), 7.97 (dd, 1H), 7.72-7.68 (m, 1H), 7.52 (t, 1H), 3.97 (s, 3H).

Step iii. A suspension of methyl 3-chloro-1-(difluoromethyl)-1H-indazole-5-carboxylate (127.3 mg, 0.49 mmol) in aq. NaOH solution (1M, 2.5 mL) was stirred vigorously overnight. The reaction mixture was cooled to 0-5° C. and aq. HCl solution (1N, ˜2.5 mL) was added to adjust pH to ˜5-6. The resulting suspension was stirred, filtered, rinsed with water and dried by suction to give 3-chloro-1-(difluoromethyl)-1H-indazole-5-carboxylic acid (VIfzq) (113.7 mg, 94% yield) as a white solid. LCMS m/z found 247.0 [M+H]⁺; RT=2.69 min (Method C); ¹H NMR (400 MHz, DMSO-d₆) δ 13.37 (s, 1H), 8.35 (dd, 1H), 8.28 (t, 1H), 8.23 (dd, 1H), 8.02 (d, 1H).

Step iv. 3-Chloro-2-(difluoromethyl)-2H-indazole-5-carboxylate (110.1 mg, 0.42 mmol) was reacted in an analogous manner as described above, without purification, to give a crude mixture of 2-(difluoromethyl)-3-methoxy-2H-indazole-5-carboxylic acid (VIfzs), 3-methoxy-1H-indazole-5-carboxylic acid (VIfzt) and desired 3-chloro-2-(difluoromethyl)-2H-indazole-5-carboxylic acid (VIfzr) (77 mg, 74% yield) as a tan solid. LCMS m/z found 247.0 [M+H]⁺; RT=2.45 min (Method C).

### (S)-3-Chloro-N-(8,9-difluoro-6-oxo-1,4,5,6-tetrahydro-2H-pyrano[3,4-c]isoquinolin-1-yl)-1(difluoromethyl)-N-methyl-1H-indazole-5-carboxamide (Compound 359)

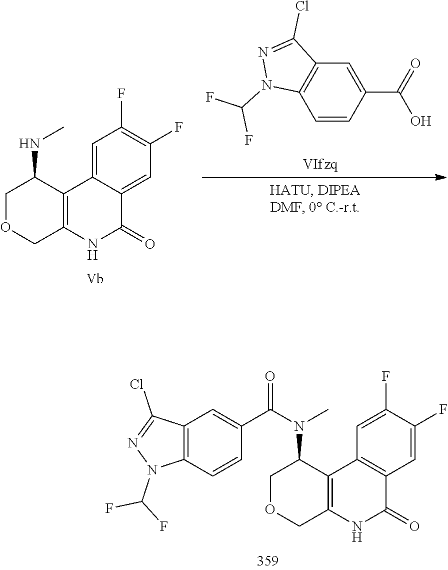

Enantiomerically pure (S)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 3-chloro-1-(difluoromethyl)-1H-indazole-5-carboxylic acid (VIfzq). LCMS m/z found 495.1 [M+H]⁺; RT=2.60 min (Method D); ¹H NMR (400 MHz, DMSO-d₆) δ 11.72 (s, 1H), 8.41-8.09 (m, 2H), 7.99 (d, 1H), 7.93 (s, 1H), 7.72 (dd, 1H), 7.58 (dd, 1H), 5.73 (s, 1H), 4.60 (d, 1H), 4.48 (d, 1H), 4.37 (d, 1H), 4.04 (dd, 1H), 2.74 (s, 3H).

### (S)-3-Chloro-N-(8,9-difluoro-6-oxo-1,4,5,6-tetrahydro-2H-pyrano[3,4-c]isoquinolin-1-yl)-2-(difluoromethyl)-N-methyl-2H-indazole-5-carboxamide (Compound 360)

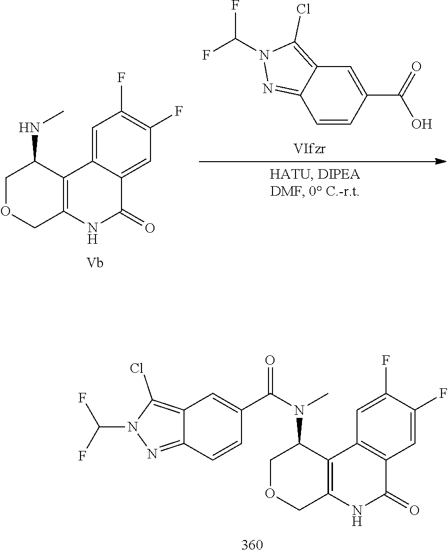 [SMILES: CN[C@@H]1COCc2[nH]c(=O)c3cc(F)c(F)cc3c21 ; O=C(O)c1ccc2nn(C(F)F)c(Cl)c2c1 ; CN(C(=O)c1ccc2nn(C(F)F)c(Cl)c2c1)[C@@H]1COCc2[nH]c(=O)c3cc(F)c(F)cc3c21]

Enantiomerically pure (S)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-5-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and crude 3-chloro-2-(difluoromethyl)-2H-indazole-5-carboxylic acid (VIfzr). LCMS m/z found 495.1 [M+H]⁺; RT=2.39 min (Method D); ¹H NMR (400 MHz, DMSO-d₆) δ 11.64 (s, 1H), 8.43-8.09 (m, 2H), 7.81 (d, 1H), 7.78 (s, 1H), 7.54 (dd, 1H), 7.39 (d, 1H), 5.70 (s, 1H), 4.58 (d, 1H), 4.45 (d, 1H), 4.32 (d, 1H), 4.05-3.98 (m, 1H), 2.74 (s, 3H).

### (S)-N-(8,9-Difluoro-6-oxo-1,4,5,6-tetrahydro-2H-pyrano[3,4-c]isoquinolin-1-yl)-2-(difluoromethyl)-3-methoxy-N-methyl-2H-indazole-6-carboxamide (Compound 367)

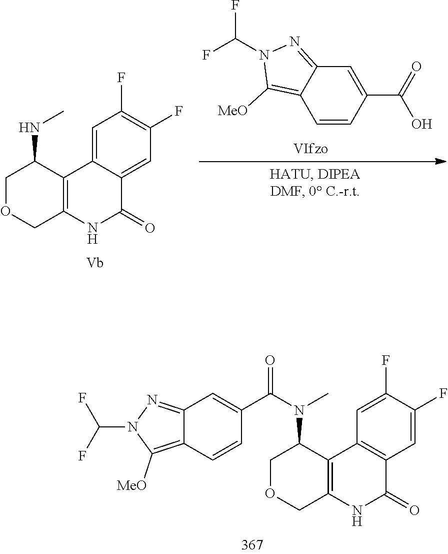 [SMILES: CN[C@@H]1COCc2[nH]c(=O)c3cc(F)c(F)cc3c21 ; COc1c2ccc(C(=O)O)cc2nn1C(F)F ; COc1c2ccc(C(=O)N(C)[C@@H]3COCc4[nH]c(=O)c5cc(F)c(F)cc5c43)cc2nn1C(F)F]

Enantiomerically pure (S)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-6-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and crude 2-(difluoromethyl)-3-methoxy-2H-indazole-6-carboxylic acid (VIfzo). LCMS m/z found 491.1 [M+H]⁺; RT=2.15 min (Method D); ¹H NMR (400 MHz, DMSO-d₆) δ 11.64 (s, 1H), 8.20-8.05 (m, 1H), 8.04-7.78 (m, 2H), 7.58-7.41 (m, 2H), 6.93-6.82 (m, 1H), 5.70 (s, 1H), 4.59 (d, 1H), 4.52-4.37 (m, 4H), 4.30 (d, 1H), 4.12-3.99 (m, 1H), 2.74 (s, 3H).

### (S)-N-(8,9-Difluoro-6-oxo-1,4,5,6-tetrahydro-2H-pyrano[3,4-c]isoquinolin-1-yl)-2-(difluoromethyl)-3-methoxy-N-methyl-2H-indazole-5-carboxamide (Compound 368)

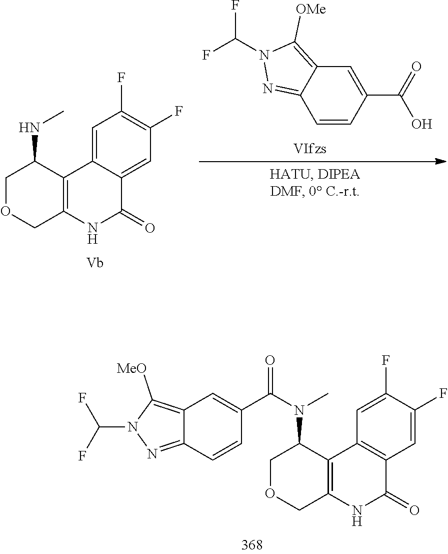 [SMILES: CN[C@@H]1COCc2[nH]c(=O)c3cc(F)c(F)cc3c21 ; COc1c2cc(C(=O)O)ccc2nn1C(F)F ; COc1c2cc(C(=O)N(C)[C@@H]3COCc4[nH]c(=O)c5cc(F)c(F)cc5c43)ccc2nn1C(F)F]

Enantiomerically pure (S)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-5-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and crude 2-(difluoromethyl)-3-methoxy-2H-indazole-5-carboxylic acid (VIfzs). LCMS m/z found 491.1 [M+H]⁺; RT=2.12 min (Method D); ¹H NMR (400 MHz, DMSO-d₆) δ 11.72 (s, 1H), 8.18-7.79 (m, 3H), 7.59-7.49 (m, 2H), 7.21 (d, 1H), 5.70 (s, 1H), 4.60 (d, 1H), 4.46 (d, 4H), 4.29 (d, 1H), 4.09-3.98 (m, 1H), 2.78 (s, 3H).

### (S)-N-(8,9-Difluoro-6-oxo-1,4,5,6-tetrahydro-2H-pyrano[3,4-c]isoquinolin-1-yl)-3-methoxy-N-methyl-1H-indazole-5-carboxamide (Compound 369)

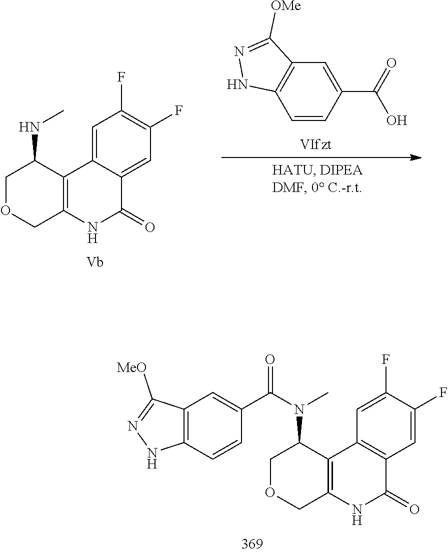 [SMILES: CN[C@@H]1COCc2[nH]c(=O)c3cc(F)c(F)cc3c21 ; COc1n[nH]c2ccc(C(=O)O)cc12 ; COc1n[nH]c2ccc(C(=O)N(C)[C@@H]3COCc4[nH]c(=O)c5cc(F)c(F)cc5c43)cc12]

Enantiomerically pure (S)-N-(8,9-difluoro-6-oxo-1,4,5,6-tetrahydro-2H-pyrano[3,4-c]isoquinolin-1-yl)-3-methoxy-N-methyl-1H-indazole-5-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and crude 3-methoxy-1H-indazole-5-carboxylic acid (VIfzt). LCMS m/z found 441.2 [M+H]⁺; RT=1.85 min (Method D); ¹H NMR (400 MHz, DMSO-d₆) δ 12.18 (s, 1H), 11.70 (s, 1H), 8.18-8.10 (m, 1H), 7.65 (s, 1H), 7.61-7.52 (m, 1H), 7.42 (d, 1H), 7.37 (d, 1H), 5.70 (s, 1H), 4.59 (d, 1H), 4.46 (d, 1H), 4.29 (d, 1H), 4.07-4.01 (m, 1H), 4.00 (s, 3H), 2.76 (s, 3H).

### 1-(5-Chlorothiophen-3-yl)azetidine-3-carboxylic acid (VIfzu)

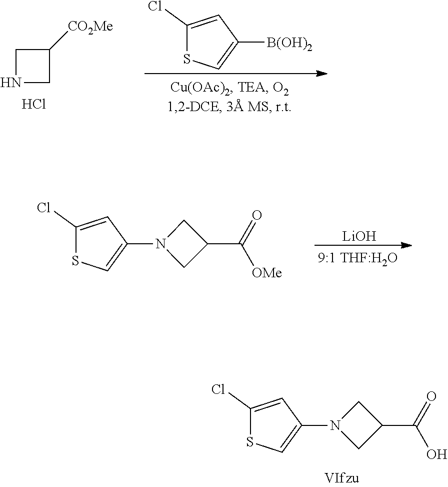 [SMILES: COC(=O)C1CNC1 ; OB(O)c1csc(Cl)c1 ; Cl ; COC(=O)C1CN(c2csc(Cl)c2)C1 ; "LiOH" ; O=C(O)C1CN(c2csc(Cl)c2)C1]

Step i. A mixture of methyl azetidine-3-carboxylate hydrochloride (1.0 g, 6.6 mmol), (5-chloro-3-thienyl)boronic acid (1.3 g, 7.9 mmol), Copper(II) acetate (1.8 g, 9.9 mmol), TEA (4.6 mL, 33 mmol) and 3 Å MS (2.0 g) in dry 1,2-DCE (30 mL) was degassed under vacuum. The reaction was placed under an Oxygen atmosphere (via balloon) and the dark blue mixture was stirred at room temperature for 18 hours. The blue-green suspension was filtered through a plug of CELITE® and the filter cake was rinsed with CH₂Cl₂. The green filtrate was evaporated and purified via normal phase SiO₂ chromatography (0-50% EtOAc/Hexanes). The desired fractions were collected and evaporated under reduced pressure to give methyl 1-(5-chlorothiophen-3-yl)azetidine-3-carboxylate (103 mg, 6% yield) as a tan oil. LCMS m/z found 232.1 [M+H]⁺; RT=3.00 min (Method C); ¹H NMR (400 MHz, CDCl₃) δ 6.41 (d, 1H), 5.68 (d, 1H), 4.03-3.92 (m, 4H), 3.74 (s, 3H), 3.57-3.48 (m, 1H).

Step ii. In a 1-dram vial, to a stirred mixture of methyl 1-(5-chlorothiophen-3-yl)azetidine-3-carboxylate (103 mg, 0.44 mmol) in THF-MeOH (9:1, 1 mL) was added LiOH (13 mg, 0.53 mmol). The suspension was stirred at room temperature overnight. The reaction mixture was evaporated to dryness to give crude 1-(5-chlorothiophen-3-yl)azetidine-3-carboxylic acid (VIfzu) (101 mg), as the lithium salt, as a red-brown solid. LCMS m/z found 218.0 [M+H]⁺; RT=2.63 min (Method C); ¹H NMR (400 MHz, DMSO-d₆) δ 6.60-6.58 (m, 1H), 5.79-5.76 (m, 1H), 3.73-3.68 (m, 2H), 3.67-3.62 (m, 2H), 2.96-2.86 (m, 1H).

### (S)-1-(5-Chlorothiophen-3-yl)-N-(8,9-difluoro-6-oxo-1,4,5,6-tetrahydro-2H-pyrano[3,4-c]isoquinolin-1-yl)-N-methylazetidine-3-carboxamide (Compound 370)

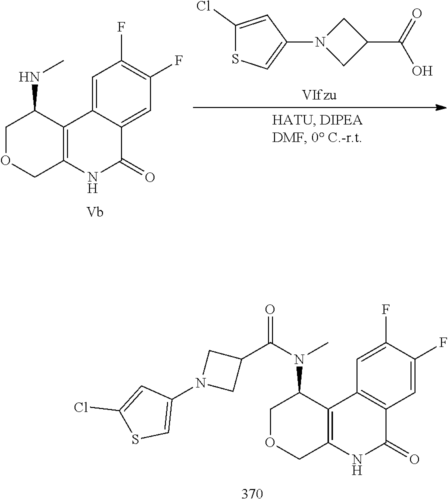 [SMILES: CN[C@@H]1COCc2[nH]c(=O)c3cc(F)c(F)cc3c21 ; O=C(O)C1CN(c2csc(Cl)c2)C1 ; CN(C(=O)C1CN(c2csc(Cl)c2)C1)[C@@H]1COCc2[nH]c(=O)c3cc(F)c(F)cc3c21]

Enantiomerically pure (S)-1-(5-chlorothiophen-3-yl)-N-(8,9-difluoro-6-oxo-1,4,5,6-tetrahydro-2H-pyrano[3,4-c]isoquinolin-1-yl)-N-methylazetidine-3-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 1-(5-chlorothiophen-3-yl)azetidine-3-carboxylic acid (VIfzu). LCMS m/z found 466.1 [M+H]⁺; RT=2.63 min (Method D); ¹H NMR (400 MHz, DMSO-d₆) δ 11.68 (s, 1H), 8.10 (dd, 1H), 7.24 (dd, 1H), 6.72 (d, 1H), 5.98 (d, 1H), 5.55 (s, 1H), 4.57 (d, 1H), 4.42 (d, 1H), 4.07-3.83 (m, 6H), 3.71 (t, 1H), 2.69 (s, 3H).

### (S)-7-Chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide (Compound 371)

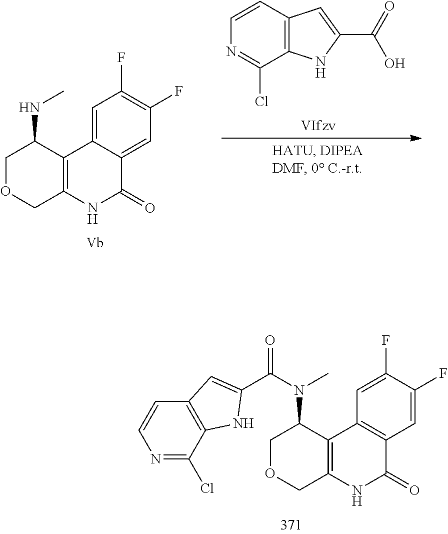 [SMILES: CN[C@@H]1COCc2[nH]c(=O)c3cc(F)c(F)cc3c21 ; O=C(O)c1cc2ccnc(Cl)c2[nH]1 ; CN(C(=O)c1cc2ccnc(Cl)c2[nH]1)[C@@H]1COCc2[nH]c(=O)c3cc(F)c(F)cc3c21]

Enantiomerically pure (S)-7-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 7-chloro-1H-pyrrolo[2,3-c]pyridine-2-carboxylic acid (VIfzv). LCMS m/z found 445.1 [M+H]⁺; RT=2.01 min (Method D); ¹H NMR (400 MHz, DMSO-d₆) δ 12.58 (s, 1H), 11.76 (s, 1H), 8.15 (dd, 1H), 7.96 (dd, 1H), 7.61 (d, 1H), 7.46 (dd, 1H), 6.94 (s, 1H), 5.72 (s, 1H), 4.63 (d, 1H), 4.49 (d, 1H), 4.24 (d, 1H), 4.05 (dd, 1H), 2.97 (s, 3H).

### (S)-4-Chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide (Compound 372)

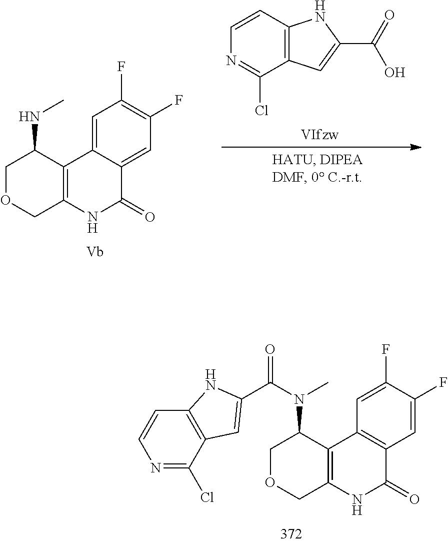 [SMILES: CN[C@@H]1COCc2[nH]c(=O)c3cc(F)c(F)cc3c21 ; O=C(O)c1cc2c(Cl)nccc2[nH]1 ; CN(C(=O)c1cc2c(Cl)nccc2[nH]1)[C@@H]1COCc2[nH]c(=O)c3cc(F)c(F)cc3c21]

Enantiomerically pure (S)-4-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 4-chloro-1H-pyrrolo[3,2-c]pyridine-2-carboxylic acid (VIfzw). LCMS m/z found 445.1 [M+H]⁺; RT=1.87 min (Method D); ¹H NMR (400 MHz, DMSO-d₆) δ 12.28 (s, 1H), 11.75 (s, 1H), 8.73 (s, 1H), 8.13 (t, 1H), 7.47-7.39 (m, 2H), 7.15 (s, 1H), 5.75 (s, 1H), 4.65 (d, 1H), 4.49 (d, 1H), 4.19 (d, 1H), 4.04 (d, 1H), 3.16 (s, 3H).

### 4-Bromo-5-chloro-1H-pyrrolo[2,3-c]pyridine-2-carboxylic acid (VIfzx)

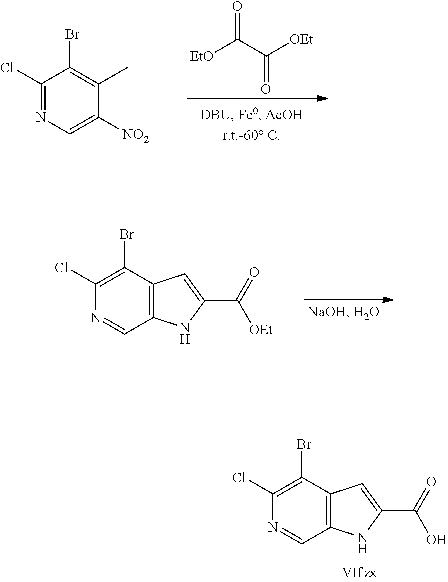 [SMILES: Cc1c([N+](=O)[O-])cnc(Cl)c1Br ; CCOC(=O)C(=O)OCC ; CCOC(=O)c1cc2c(Br)c(Cl)ncc2[nH]1 ; O=C(O)c1cc2c(Br)c(Cl)ncc2[nH]1]

Step i. To a solution of 3-bromo-2-chloro-4-methyl-5-nitro-pyridine (1.2 g, 4.9 mmol) and diethyl oxalate (3.0 mL, 22 mmol) at room temperature was added dropwise DBU (0.80 mL, 5.3 mmol). The reaction mixture was stirred at room temperature under nitrogen overnight. The reaction mixture was evaporated at 70° C. water bath temperature under reduced pressure. The residue was dissolved in AcOH (10 mL) and warmed to 60° C. then powdered iron, (0.54 g, 9.7 mmol) was added. The reaction mixture was stirred vigorously under nitrogen and heated at 70° C. for 2 h. The reaction mixture was cooled to room temperature, diluted with water (30 mL) and the resulting precipitate was filtered and rinsed with water and partially dried by suction. The crude, red-brown solid was purified via normal phase SiO₂ chromatography (0-10% MeOH/CH₂Cl₂). The desired fractions were collected and evaporated to give a dark-yellow solid (112 mg) with impurities. The recovered solid was repurified via normal phase SiO₂ chromatography (2% MeOH/CH₂Cl₂ isocratic elution). The desired fraction was collected and evaporated under reduced pressure to give ethyl 4-bromo-5-chloro-1H-pyrrolo[2,3-c]pyridine-2-carboxylate (46.6 mg, 3% yield) as light yellow solid. LCMS m/z found 305.0 [M+H]⁺; RT=3.16 min (Method D); ¹H NMR (400 MHz, DMSO-d₆) δ 13.04 (s, 1H), 8.67-8.62 (m, 1H), 7.08-7.04 (m, 1H), 4.40 (q, 2H), 1.37 (t, 3H).

Step ii. A suspension of ethyl 4-bromo-5-chloro-1H-pyrrolo[2,3-c]pyridine-2-carboxylate (46.6 mg, 0.15 mmol) in aq. NaOH solution (1M, 0.75 mL) and MeOH (0.25 mL) was stirred at room temperature overnight. The reaction mixture was cooled to 5° C. and acidified with aq. HCl solution (1M, ˜1 mL). The mixture was stirred for 10 minutes then extracted with EtOAc (50 mL). The layers were separated. The aqueous layer contained desired product and was extracted with CHCl₃-MeOH (9:1 v/v, 3×10 mL). The organic chloroform layers were combined and dried over Na₂SO₄, filtered and evaporated then subjected to high vacuum for 1 hour to give crude 4-bromo-5-chloro-1H-pyrrolo[2,3-c]pyridine-2-carboxylic acid (VIfzx) (44 mg) as a yellow solid. LCMS m/z found 276.9 [M+H]⁺; RT=2.58 min (Method D); ¹H NMR (400 MHz, DMSO-d₆) δ 12.17 (s, 1H), 10.26 (s, 1H), 8.45 (s, 1H), 6.62 (s, 1H).

### (S)-4-Bromo-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide (Compound 384)

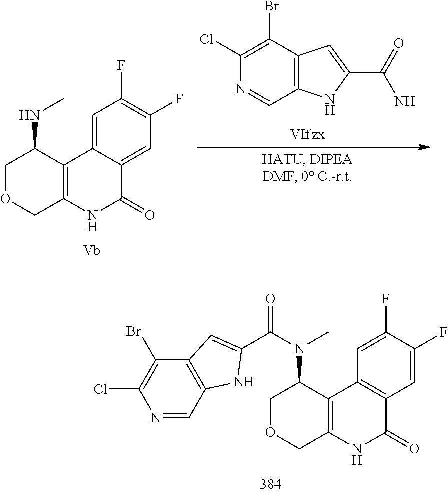 [SMILES: CN[C@@H]1COCc2[nH]c(=O)c3cc(F)c(F)cc3c21 ; NC(=O)c1cc2c(Br)c(Cl)ncc2[nH]1 ; CN(C(=O)c1cc2c(Br)c(Cl)ncc2[nH]1)[C@@H]1COCc2[nH]c(=O)c3cc(F)c(F)cc3c21]

Enantiomerically pure 4-bromo-5-chloro-N-[(1S)-8,9-difluoro-6-oxo-1,2,4,5-tetrahydropyrano[3,4-c]isoquinolin-1-yl]-N-methyl-1H-pyrrolo[2,3-c]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 4-bromo-5-chloro-1H-pyrrolo[2,3-c]pyridine-2-carboxylic acid (VIfzx). LCMS m/z found 523.0 [M+H]⁺; RT=2.55 min (Method D); ¹H NMR (400 MHz, DMSO-d₆) δ 12.74 (br s, 1H), 11.73 (br s, 1H) 8.62 (s, 1H), 8.17-8.11 (m, 1H), 7.49-7.41 (m, 1H), 6.89 (s, 1H), 5.73 (s, 1H), 4.64 (d, 1H), 4.49 (d, 1H), 4.24 (d, 1H), 4.07-4.01 (m, 1H), 3.12 (s, 3H).

### (S)-4,6-Dichloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide (Compound 410)

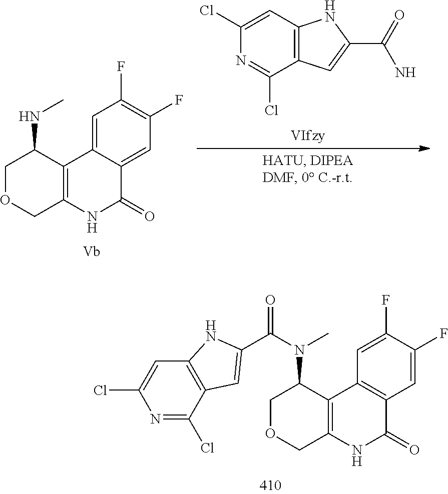 [SMILES: CN[C@@H]1COCc2[nH]c(=O)c3cc(F)c(F)cc3c21 ; NC(=O)c1cc2c(Cl)nc(Cl)cc2[nH]1 ; CN(C(=O)c1cc2c(Cl)nc(Cl)cc2[nH]1)[C@@H]1COCc2[nH]c(=O)c3cc(F)c(F)cc3c21]

Enantiomerically pure (S)-4,6-dichloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 4,6-dichloro-1H-pyrrolo[3,2-c]pyridine-2-carboxylic acid (VIfzy). LCMS m/z found 479.1 [M+H]⁺; RT=2.55 min (Method D); ¹H NMR (400 MHz, DMSO-d₆) δ 12.69 (s, 1H), 11.75 (s, 1H), 8.13 (dd, 1H), 7.47 (s, 1H), 7.44 (dd, 1H), 7.08 (s, 1H), 5.73 (s, 1H), 4.64 (d, 1H), 4.49 (d, 1H), 4.21 (d, 1H), 4.03 (dd, 1H), 3.16 (s, 3H).

### 3-Chloro-1-methyl-1H-indazole-5-carboxylic acid (VIfzz)

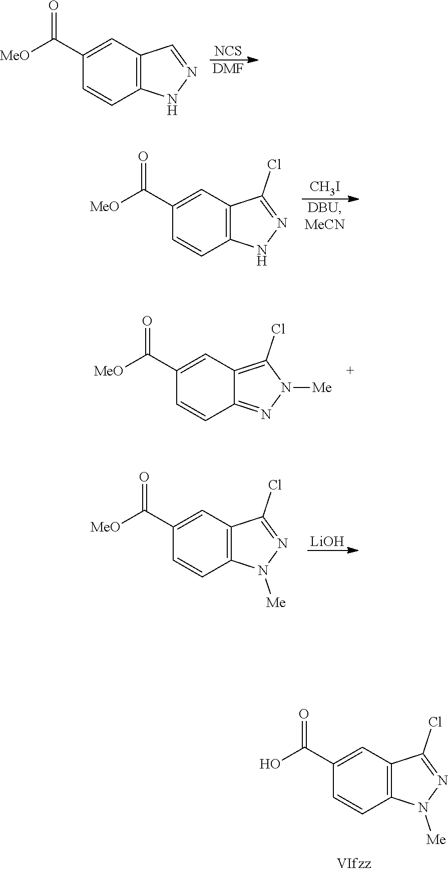

Step i. To a stirred suspension of methyl 3-chloro-1H-indazole-5-carboxylate (250.0 mg, 1.19 mmol) in dry MeCN (3 mL) was added dropwise DBU (195 uL, 1.31 mmol) and stirred for 15 minutes at room temperature then iodomethane (81 uL, 1.3 mmol) was added and the mixture was stirred overnight. The reaction mixture was poured into water (25 mL) and extracted with EtOAc (2×25 mL). The combined organic was dried over MgSO₄, filtered and evaporated. The residue was purified via normal phase SiO₂ chromatography (0-50% EtOAc/Hexanes). The desired fractions were collected and evaporated under reduced pressure to give methyl 3-chloro-1-methyl-1H-indazole-5-carboxylate (203.2 mg, 76% yield) as an off-white solid, first eluted peak. LCMS m/z found 225.0 [M+H]⁺; RT=2.93 min (Method C); ¹H NMR (400 MHz, CDCl₃) δ 8.45 (dd, 1H), 8.11 (dd, 1H), 7.39 (dd, 1H), 4.06 (s, 3H), 3.96 (s, 3H).

Methyl 3-chloro-2-methyl-2H-indazole-5-carboxylate (10.2 mg, 3.8% yield) as an off-white solid, second eluted peak. LCMS m/z found 225.0 [M+H]⁺; RT=2.67 min (Method C); ¹H NMR (400 MHz, CDCl₃) δ 8.41 (dd, 1H), 7.92 (dd, 1H), 7.64 (dd, 1H), 4.19 (s, 3H), 3.95 (s, 3H).

Step ii. Lithium hydroxide (7.0 mg, 0.29 mmol) was added to a stirred mixture of methyl 3-chloro-1-methyl-1H-indazole-5-carboxylate (55 mg, 0.24 mmol) in THF-MeOH (9:1, 1 mL) and was stirred at room temperature overnight. Additional LiOH (3 mg) was added and stirring continued for 24 hours. The volatiles were evaporated to give 3-chloro-1-methyl-1H-indazole-5-carboxylic acid (VIfzz) (70 mg, >100% yield), as the lithium salt, as an off-white solid. LCMS m/z found 211.0 [M+H]⁺; RT=2.40 min (Method C); ¹H NMR (400 MHz, DMSO-d₆) δ 8.11 (dd, 1H), 8.06 (dd, 1H), 7.49 (dd, 1H), 3.99 (s, 3H).

### (S)-3-Chloro-N-(8,9-difluoro-6-oxo-1,4,5,6-tetrahydro-2H-pyrano[3,4-c]isoquinolin-1-yl)-N,1-dimethyl-1H-indazole-5-carboxamide (Compound 415)

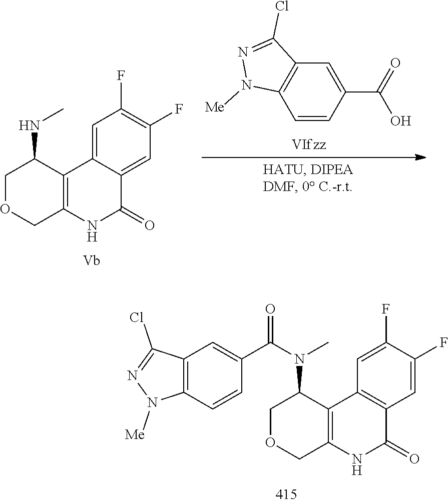

Enantiomerically pure (S)-3-chloro-N-(8,9-difluoro-6-oxo-1,4,5,6-tetrahydro-2H-pyrano[3,4-c]isoquinolin-1-yl)-N,1-dimethyl-1H-indazole-5-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 3-chloro-1-methyl-indazole-5-carboxylic acid (VIfzz). LCMS m/z found 459.2 [M+H]⁺; RT=2.31 min (Method D); ¹H NMR (400 MHz, DMSO-d₆) δ 11.71 (s, 1H), 8.17-8.10 (m, 1H), 7.79 (d, 1H), 7.74 (s, 1H), 7.58 (dd, 1H), 7.51 (d, 1H), 5.72 (s, 1H), 4.59 (d, 1H), 4.47 (d, 1H), 4.34 (d, 1H), 4.05 (s, 4H), 2.74 (s, 3H).

### 3-Chloro-1-ethyl-1H-indazole-5-carboxylic acid (VIfzza)

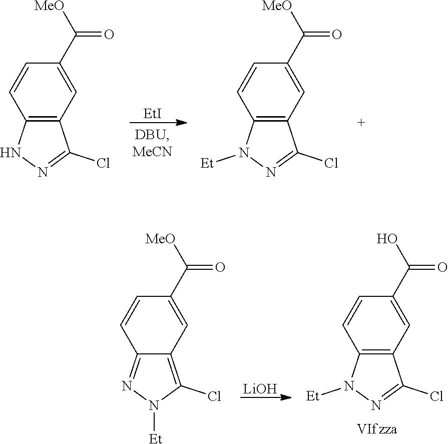

Step i. To a stirred suspension of methyl 3-chloro-1H-indazole-5-carboxylate (250 mg, 1.19 mmol) in dry MeCN (3 mL) was added dropwise DBU (195 uL, 1.31 mmol) and stirred for 15 minutes at room temperature then iodoethane (105 uL, 1.31 mmol) was added and the mixture was stirred at room temperature overnight. The reaction mixture was poured into water (25 mL) and extracted with EtOAc (2×25 mL). The combined organic was dried over MgSO₄, filtered and evaporated. The residue was purified via normal phase SiO₂ chromatography (0-50% EtOAc/Hexanes). The desired fractions were collected and evaporated to give methyl 3-chloro-1-ethyl-1H-indazole-5-carboxylate (216.6 mg, 76% yield) as an off-white solid, first eluted peak. LCMS m/z found 239.0 [M+H]⁺; RT=3.13 min (Method C); ¹H NMR (400 MHz, CDCl₃) δ 8.45 (dd, 1H), 8.10 (dd, 1H), 7.40 (dd, 1H), 4.39 (q, 2H), 3.96 (s, 3H), 1.53 (t, 3H).

Methyl 3-chloro-2-ethyl-2H-indazole-5-carboxylate (32.7 mg, 11% yield) as a clear resin, second eluted peak. LCMS m/z found 239.0 [M+H]⁺; RT=2.89 min (Method C); ¹H NMR (400 MHz, CDCl₃) δ 8.42-8.40 (m, 1H), 7.94-7.89 (m, 1H), 7.65 (d, 1H), 4.52 (q, 2H), 3.95 (s, 3H), 1.62-1.57 (m, 3H).

Step ii. Lithium hydroxide (6.6 mg, 0.28 mmol) was added to stirred mixture of methyl 3-chloro-1-ethyl-1H-indazole-5-carboxylate (55 mg, 0.23 mmol) in THF-MeOH (9:1, 1 mL). The mixture was stirred at room temperature overnight. Additional LiOH (3 mg) was added and stirring continued for 24 hours. The volatiles were evaporated to give crude 3-chloro-1-ethyl-1H-indazole-5-carboxylic acid (VIfzza) (68 mg, >100% yield), as the lithium salt, as an off-white solid. LCMS m/z found 225.0 [M+H]⁺; RT=2.61 min (Method C); ¹H NMR (400 MHz, DMSO-d₆) δ 8.07 (s, 1H), 8.02 (d, 1H), 7.50 (d, 1H), 4.36 (q, 2H), 1.36 (t, 3H).

### (S)-3-Chloro-N-(8,9-difluoro-6-oxo-1,4,5,6-tetrahydro-2H-pyrano[3,4-c]isoquinolin-1-yl)-1-ethyl-N-methyl-1H-indazole-5-carboxamide (Compound 416)

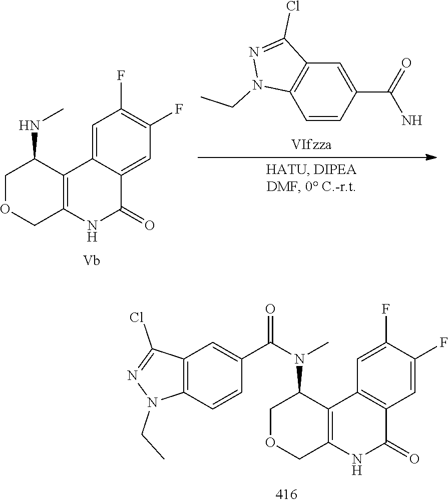

Enantiomerically pure (S)-3-chloro-N-(8,9-difluoro-6-oxo-1,4,5,6-tetrahydro-2H-pyrano[3,4-c]isoquinolin-1-yl)-1-ethyl-N-methyl-1H-indazole-5-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 3-chloro-1-ethyl-1H-indazole-5-carboxylic acid (VIfzza). LCMS m/z found 473.1 [M+H]⁺; RT=2.54 min (Method D); ¹H NMR (400 MHz, DMSO-d₆) δ 11.71 (s, 1H), 8.18-8.11 (m, 1H), 7.83 (d, 1H), 7.74 (s, 1H), 7.58 (dd, 1H), 7.50 (d, 1H), 5.72 (s, 1H), 4.60 (d, 1H), 4.51-4.40 (m, 3H), 4.34 (d, 1H), 4.08-4.00 (m, 1H), 2.75 (s, 3H), 1.40 (t, 3H).

### 1-(Difluoromethyl)-3-methyl-1H-indazole-5-carboxylic acid (VIfzzb)

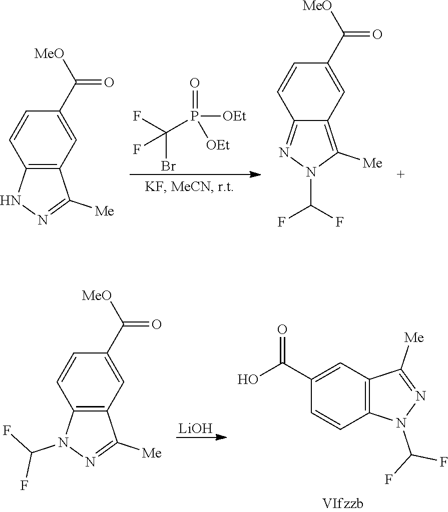

Step i. To a dry scintillation vial with stir bar and cap with rubber septa was added methyl 3-methyl-1H-indazole-5-carboxylate (250 mg, 1.3 mmol) and anhydrous potassium fluoride (305 mg, 5.26 mmol) and the vial was sealed and held under nitrogen atmosphere.

Dry acetonitrile (5 mL) was added via syringe. The mixture was stirred vigorously for 5 minutes then diethyl(bromodifluoromethyl)phosphonate (0.35 mL, 2.0 mmol) was added via syringe. The mixture was stirred at room temperature, under nitrogen, overnight. The mixture was filtered through a plug of CELITE® and rinsed with EtOAc. The filtrate was evaporated under reduced pressure and the residue was purified via normal phase SiO₂ chromatography (0-50% EtOAc/Hexanes). The desired fractions were collected and evaporated to give methyl 1-(difluoromethyl)-3-methyl-1H-indazole-5-carboxylate (11.8 mg, 3.8% yield) as an off-white solid, first eluted peak. LCMS m/z found 241.0 [M+H]⁺; RT=2.94 min (Method C); ¹H NMR (400 MHz, CDCl₃) δ 8.46 (dd, 1H), 8.17 (dd, 1H), 7.73 (dd, 1H), 7.40 (t, 1H), 3.97 (s, 3H), 2.62-2.60 (m, 3H). Methyl 2-(difluoromethyl)-3-methyl-2H-indazole-5-carboxylate (276.7 mg, 87% yield) as an off-white solid, second eluted peak. LCMS m/z found 241.0 [M+H]⁺; RT=2.76 min (Method C); ¹H NMR (400 MHz, CDCl₃) δ 8.47 (dd, 1H), 7.92 (dd, 1H), 7.64 (dd, 1H), 7.49 (t, 1H), 3.95 (s, 3H), 2.88-2.85 (m, 3H).

Step ii. Lithium hydroxide (1.4 mg, 0.06 mmol) was added to a stirred mixture of methyl 1-(difluoromethyl)-3-methyl-1H-indazole-5-carboxylate (11.8 mg, 0.05 mmol) in THF-MeOH (9:1, 1 mL). The mixture was stirred at room temperature overnight. Additional LiOH (1 mg) was added and stirring continued for 24 h. The volatiles were evaporated to give crude 1-(difluoromethyl)-3-methyl-1H-indazole-5-carboxylic acid (VIfzzb) (15.7 mg, >100% yield), as the lithium salt, as an off-white solid. LCMS m/z found 227.1 [M+H]⁺; RT=2.48 min (Method C); ¹H NMR (400 MHz, DMSO-d₆) δ 8.24-8.22 (m, 1H), 8.10 (dd, 1H), 8.03 (t, 1H), 7.57 (d, 1H), 2.52 (s, 3H).

### (S)-N-(8,9-Difluoro-6-oxo-1,4,5,6-tetrahydro-2H-pyrano[3,4-c]isoquinolin-1-yl)-1-(difluoromethyl)-N,3-dimethyl-1H-indazole-5-carboxamide (Compound 417)

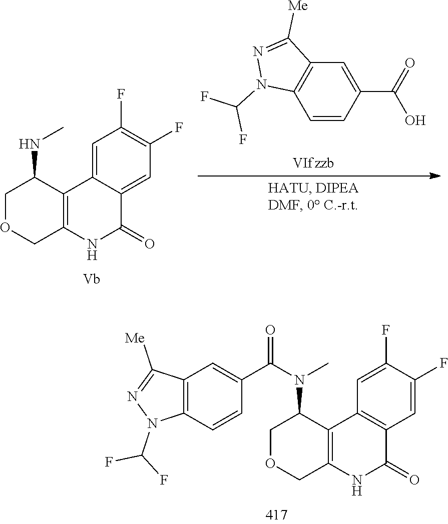

Enantiomerically pure (S)-N-(8,9-difluoro-6-oxo-1,4,5,6-tetrahydro-2H-pyrano[3,4-c]isoquinolin-1-yl)-1-(difluoromethyl)-N,3-dimethyl-1H-indazole-5-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 1-(difluoromethyl)-3-methyl-1H-indazole-5-carboxylic acid (VIfzzb). LCMS m/z found 475.2 [M+H]⁺; RT=2.40 min (Method D); ¹H NMR (400 MHz, DMSO-d₆) δ 11.72 (s, 1H), 8.28-7.82 (m, 4H), 7.62-7.55 (m, 2H), 5.74 (s, 1H), 4.60 (d, 1H), 4.48 (d, 1H), 4.31 (d, 1H), 4.09-4.02 (m, 1H), 2.76 (s, 3H), 2.56 (s, 3H).

### 3-(difluoromethylDifluoromethyldifluoromethyl)-1-methyl-1H-indazole-5-carboxylic acid (VIfzzc)

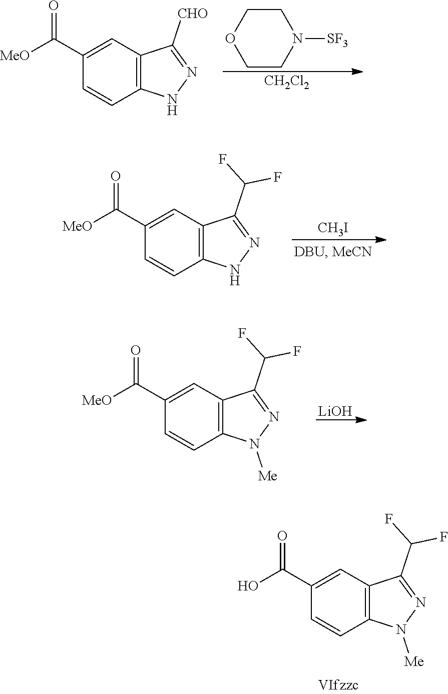

Step i. To a stirred suspension of methyl 3-formyl-1H-indazole-5-carboxylate (100 mg, 0.49 mmol) in dry CH₂Cl₂ (3 mL) was added dropwise morpholinosulfur trifluoride (179 uL, 1.47 mmol) via syringe. The mixture was stirred at room temperature for 3 days. The reaction mixture was slowly added into cold sat. aq. NaHCO₃ solution (25 mL). The mixture was stirred for 15-20 min, until all gas evolution ceased. The mixture was extracted with CH₂Cl₂ (3×10 mL). The combined organic layer was dried over Na₂SO₄, filtered and evaporated under reduced pressure. The residue was purified via normal phase SiO₂ chromatography (0-3% MeOH/CH₂Cl₂). The desired fraction was collected and evaporated under reduced pressure to give methyl 3-(difluoromethyl)-1H-indazole-5-carboxylate (51.4 mg, 46% yield) as a dark-orange solid. LCMS m/z found 227.1 [M+H]⁺; RT=2.66 min (Method C); ¹H NMR (400 MHz, DMSO-d₆) δ 13.97 (s, 1H), 8.50 (s, 1H), 8.02 (dd, 1H), 7.75 (dd, 1H), 7.45 (t, 1H), 3.89 (s, 3H).

Step ii. To a stirred suspension of methyl 3-(difluoromethyl)-1H-indazole-5-carboxylate (51.3 mg, 0.23 mmol) in dry MeCN (3 mL) was added dropwise DBU (37 uL, 0.25 mmol) and stirred for 15 minutes then iodomethane (16 uL, 0.25 mmol) was added and the mixture was stirred at room temperature overnight. The reaction mixture was poured into water (25 mL) and extracted with EtOAc (2×25 mL). The combined organic was dried over MgSO₄, filtered and evaporated. The residue was purified via normal phase SiO₂ chromatography (0-50% EtOAc/Hexanes). The desired fractions were collected and evaporated under reduced pressure to give methyl 3-(difluoromethyl)-1-methyl-1H-indazole-5-carboxylate (29.0 mg, 53% yield) as an off-white solid. LCMS m/z found 241.0 [M+H]⁺; RT=2.92 min (Method C); ¹H NMR (400 MHz, CDCl₃) δ 8.68 (dd, 1H), 8.14 (dd, 1H), 7.45 (dd, 1H), 6.97 (t, 1H), 4.12 (t, 3H), 3.97 (s, 3H).

Step iii. Lithium hydroxide (3.5 mg, 0.15 mmol) was added to a stirred mixture of methyl 3-(difluoromethyl)-1-methyl-1H-indazole-5-carboxylate (29.0 mg, 0.12 mmol) in THF-MeOH (9:1, 1 mL). The mixture was stirred at room temperature for 6 days. The volatiles were evaporated under reduced pressure to give crude 3-(difluoromethyl)-1-methyl-1H-indazole-5-carboxylic acid (VIfzzc) (32.5 mg, quantitative), as the lithium salt, as a tan solid. LCMS m/z found 227.1 [M+H]⁺; RT=2.36 min (Method C); ¹H NMR (400 MHz, DMSO-d₆) δ 8.32-8.29 (m, 1H), 8.05 (dd, 1H), 7.52 (dd, 1H), 7.31 (t, 1H), 4.08-4.05 (m, 3H).

### (S)-N-(8,9-Difluoro-6-oxo-1,4,5,6-tetrahydro-2H-pyrano[3,4-c]isoquinolin-1-yl)-3-(difluoromethyl)-N,1-dimethyl-1H-indazole-5-carboxamide (Compound 436)

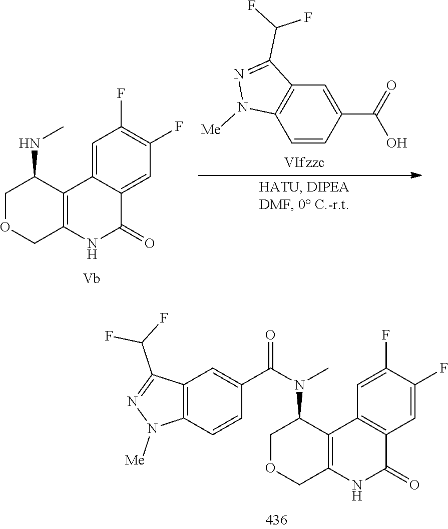

Enantiomerically pure (S)-N-(8,9-difluoro-6-oxo-1,4,5,6-tetrahydro-2H-pyrano[3,4-c]isoquinolin-1-yl)-3-(difluoromethyl)-N,1-dimethyl-1H-indazole-5-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 3-(difluoromethyl)-1-methyl-1H-indazole-5-carboxylic acid (VIfzzc). LCMS m/z found 475.2 [M+H]⁺; RT=2.26 min (Method D); ¹H NMR (400 MHz, DMSO-d₆) δ 11.71 (s, 1H), 8.18-8.11 (m, 1H), 7.90 (s, 1H), 7.84 (d, 1H), 7.60-7.25 (m, 3H), 5.72 (s, 1H), 4.59 (d, 1H), 4.48 (d, 1H), 4.34 (d, 1H), 4.13 (s, 3H), 4.09-4.00 (m, 1H), 2.74 (s, 3H).

### 1-(Difluoromethyl)-1H-indazole-5-carboxylic acid (VIfzzd)

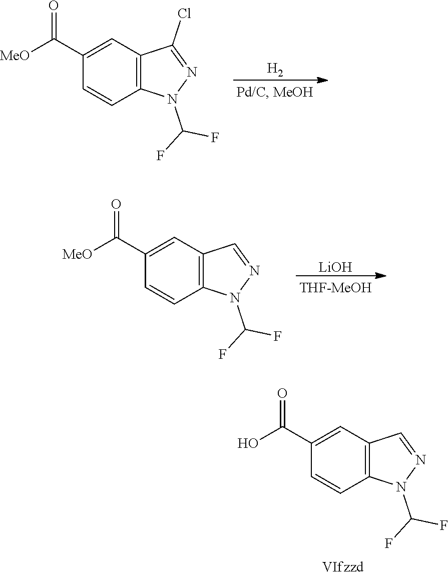 [SMILES: COC(=O)c1ccc2c(c1)c(Cl)nn2C(F)F ; "H2" ; COC(=O)c1ccc2c(cnn2C(F)F)c1 ; "LiOH" ; O=C(O)c1ccc2c(cnn2C(F)F)c1]

Step i. A suspension of methyl 3-chloro-1-(difluoromethyl)-1H-indazole-5-carboxylate (50.00 mg, 0.19 mmol) and palladium on carbon (10 wt %) (10.2 mg, 0.01 mmol) in MeOH (2 mL) was stirred under Hydrogen (1 atm, via balloon) for 18 hours. The suspension was filtered through a plug of CELITE®, rinsed with CH₂Cl₂, and the filtrate was evaporated under reduced pressure to give methyl 1-(difluoromethyl)-1H-indazole-5-carboxylate (43 mg, 99% yield) as a tan solid. LCMS m/z found 227.0 [M+H]⁺; RT=2.75 min (Method C); ¹H NMR (400 MHz, CDCl₃) δ 8.55-8.53 (m, 1H), 8.22-8.18 (m, 2H), 7.81 (d, 1H), 7.48 (t, 1H), 3.97 (s, 3H).

Step ii. Lithium hydroxide (5.5 mg, 0.23 mmol) was added to a stirred mixture of methyl 1-(difluoromethyl)-1H-indazole-5-carboxylate (43 mg, 0.19 mmol) in THF-MeOH (9:1, 1 mL). The mixture was stirred at room temperature for 4 days. The volatiles were evaporated under reduced pressure to give crude 1-(difluoromethyl)-1H-indazole-5-carboxylic acid (VIfzzd) (49 mg, >100%), as the lithium salt, as a tan solid. LCMS m/z found 213.1 [M+H]⁺; RT=2.27 min (Method C); ¹H NMR (400 MHz, DMSO-d₆) δ 8.38-8.36 (m, 1H), 8.29-7.97 (m, 3H), 7.63 (d, 1H).

### (S)-N-(8,9-Difluoro-6-oxo-1,4,5,6-tetrahydro-2H-pyrano[3,4-c]isoquinolin-1-yl)-1-(difluoromethyl)-N-methyl-1H-indazole-5-carboxamide (Compound 437)

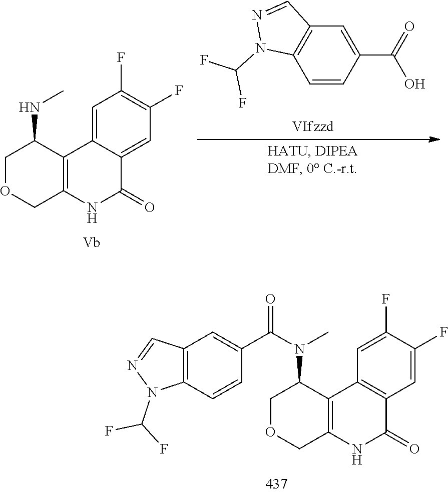

Enantiomerically pure (S)-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 1-(difluoromethyl)-1H-indazole-5-carboxylic acid (VIfzzd). LCMS m/z found 461.1 [M+H]⁺; RT=2.27 min (Method D); ¹H NMR (400 MHz, DMSO-d₆) δ 11.72 (s, 1H), 8.48 (s, 1H), 8.23 (t, 1H), 8.15 (dd, 1H), 8.00 (s, 1H), 7.92 (d, 1H), 7.64 (dd, 1H), 7.56 (dd, 1H), 5.72 (s, 1H), 4.60 (d, 1H), 4.48 (d, 1H), 4.31 (d, 1H), 4.06 (dd, 1H), 2.75 (s, 3H).

### (S)-8,9-Difluoro-1-(((R)-1-(4-methoxyphenyl)ethyl)amino)-1,5-dihydro-2H-pyrano[3,4-c]isoquinolin-6(4H)-one-1-d (VIIIb-d)

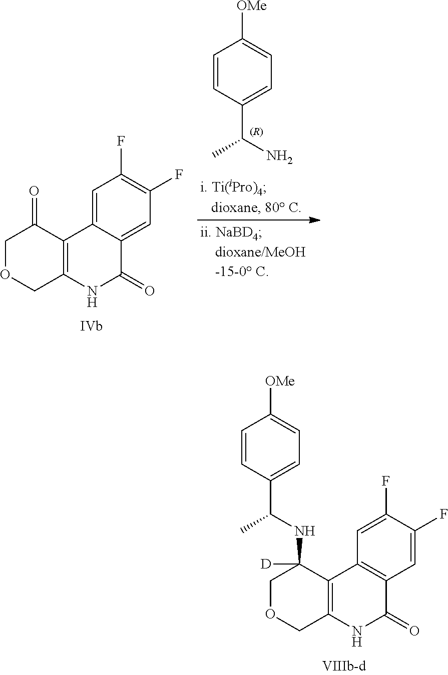

Enantiomerically pure (S)-8,9-difluoro-1-(((R)-1-(4-methoxyphenyl)ethyl)amino)-1,5-dihydro-2H-pyrano[3,4-c]isoquinolin-6(4H)-one-1-d (VIIIb-d) was synthesized in an analogous manner as described above for VIIIa, in 57% yield, starting from 8,9-difluoro-2H-pyrano[3,4-c]isoquinoline-1,6(4H,5H)-dione (IVb) followed by reduction with sodium borodeuteride. LCMS m/z found 388.2 [M+H]⁺; RT=1.55 min (Method D); ¹H NMR (400 MHz, DMSO-d₆) δ 11.35 (s, 1H), 8.01 (dd, 1H), 7.75 (dd, 1H), 7.24-7.16 (m, 2H), 6.81-6.73 (m, 2H), 4.38 (d, 1H), 4.29 (d, 1H), 4.06 (q, 1H), 4.00 (d, 1H), 3.69 (s, 3H), 3.48 (d, 1H), 2.00 (s, 1H), 1.33 (d, 3H).

### (S)-8,9-Difluoro-1-(((R)-1-(4-methoxyphenyl)ethyl)(methyl-¹³C-d₃)amino)-1,5-dihydro-2H-pyrano[3,4-c]isoquinolin-6(4H₁)-one-1-d (IXc-d)

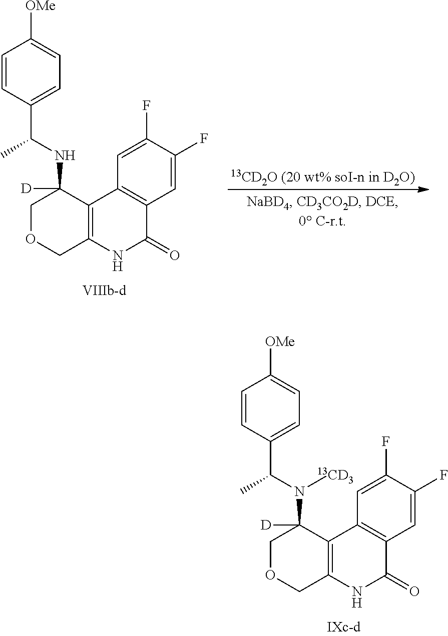

A mixture of enantiomerically pure (S)-8,9-difluoro-1-(((R)-1-(4-methoxyphenyl)ethyl)amino)-1,5-dihydro-2H-pyrano[3,4-c]isoquinolin-6(4H)-one-1-d (VIIIb-d) (457 mg, 1.18 mmol), a solution of formaldehyde-¹³C-d₂ (˜20 wt % in deuterium oxide) (0.98 mL, 5.90 mmol), acetic acid-d₄, (0.53 mL, 8.74 mmol) and sodium borodeuteride (104 mg, 2.48 mmol) were stirred in dry 1,2-dichloroethane (3 mL) at 0° C. and allowed to warm to room temperature over two hours. The reaction mixture was diluted with dichloromethane (10 mL) and neutralized with sat. aq. NaHCO₃ solution. The aqueous phase was extracted with dichloromethane twice more, and the combined extracts were dried over MgSO₄, filtered, and the solvent evaporated under reduced pressure to give enantiomerically pure (S)-8,9-difluoro-1-(((R)-1-(4-methoxyphenyl)ethyl)(methyl-¹³C-d₃)amino)-1,5-dihydro-2H-pyrano[3,4-c]isoquinolin-6(4H)-one-1-d (IXc-d) (479.4 mg, 96% yield). LCMS m/z found 406.2 [M+H]⁺; RT=1.58 min (Method D); ¹H NMR (400 MHz, DMSO-d₆) δ 11.48 (s, 1H), 8.05 (dd, 1H), 7.99 (dd, 1H), 7.18-7.13 (m, 2H), 6.85-6.80 (m, 2H), 4.45 (d, 1H), 4.31 (d, 1H), 4.23 (d, 1H), 3.98-3.86 (m, 1H), 3.70 (s, 3H), 3.54 (d, 1H), 1.41 (d, 3H).

### (S)-8,9-Difluoro-1-((methyl-¹³C-d₃)amino)-1,5-dihydro-2H-pyrano[3,4-c]isoquinolin-6(4H)-one-1-d (Vb-d)

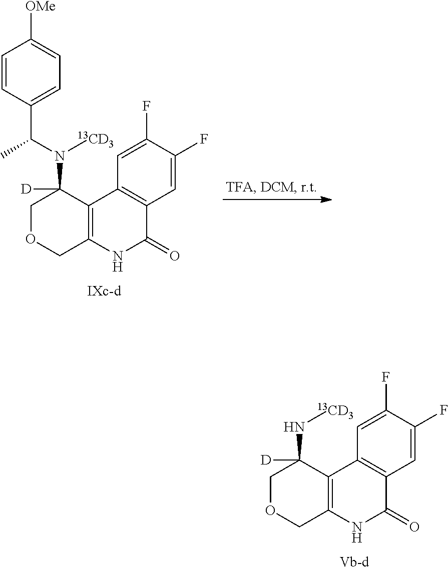

Enantiomerically pure (S)-8,9-difluoro-1-((methyl-¹³C-d₃)amino)-1,5-dihydro-2H-pyrano[3,4-c]isoquinolin-6(4H)-one-1-d (Vb-d) was synthesized in an analogous manner as described above for Vb, in 88% yield, starting from (S)-8,9-difluoro-1-(((R)-1-(4-methoxyphenyl)ethyl)(methyl-¹³C-d₃)amino)-1,5-dihydro-2H-pyrano[3,4-c]isoquinolin-6(4H)-one-1-d (IXc-d) followed by neutralization with aqueous potassium carbonate solution. LCMS m/z found 237.0 [M+H]⁺; RT=0.58 min (Method D); ¹H NMR (400 MHz, DMSO-d₆) δ 11.40 (s, 1H), 8.03 (dd, 1H), 7.75 (dd, 1H), 4.42 (d, 1H), 4.34 (d, 1H), 4.22 (d, 1H), 3.55 (d, 1H).

### (S)-N-(8,9-Difluoro-6-oxo-1,4,5,6-tetrahydro-2H-pyrano[3,4-c]isoquinolin-1-yl-1-d)-5,6-difluoro-N-(methyl-¹³C-d₃)-1H-indole-2-carboxamide (Compound 514)

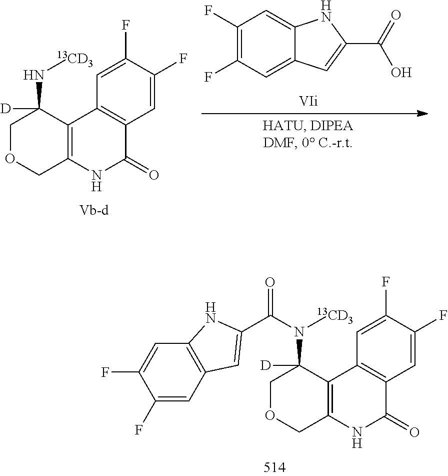

Enantiomerically pure (S)-N-(8,9-difluoro-6-oxo-1,4,5,6-tetrahydro-2H-pyrano[3,4-c]isoquinolin-1-yl-1-d)-5,6-difluoro-N-(methyl-¹³C-d₃)-1H-indole-2-carboxamide was synthesized in an analogous manner as described above from enantiomerically pure (S)-8,9-difluoro-1-((methyl-¹³C-d₃)amino)-1,5-dihydro-2H-pyrano[3,4-c]isoquinolin-6(4H)-one-1-d (Vb-d) and 5,6-difluoro-1H-indole-2-carboxylic acid (VIi). LCMS m/z found 451.2 [M+H]⁺; RT=2.65 min (Method D); ¹H NMR (400 MHz, DMSO-d₆) δ 11.92 (s, 1H), 11.74 (s, 1H), 8.13 (dd, 1H), 7.62 (dd, 1H), 7.44 (dd, 1H), 7.38 (dd, 1H), 6.97 (s, 1H), 4.64 (d, 1H), 4.48 (d, 1H), 4.16 (d, 1H), 4.03 (d, 1H).

### (S)-N-(8,9-Difluoro-6-oxo-1,4,5,6-tetrahydro-2H-pyrano[3,4-c]isoquinolin-1-yl-1-d)-6-(difluoromethyl)-5-fluoro-N-(methyl-¹³C-d₃)-1H-indole-2-carboxamide (Compound 515)

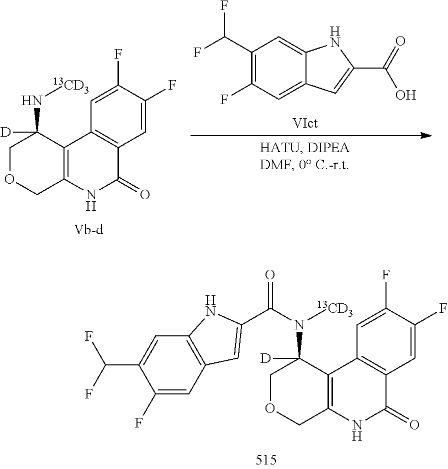

Enantiomerically pure (S)-N-(8,9-difluoro-6-oxo-1,4,5,6-tetrahydro-2H-pyrano[3,4-c]isoquinolin-1-yl-1-d)-6-(difluoromethyl)-5-fluoro-N-(methyl-¹³C-d₃)-1H-indole-2-carboxamide was synthesized in an analogous manner as described above from enantiomerically pure (S)-8,9-difluoro-1-((methyl-¹³C-d₃)amino)-1,5-dihydro-2H-pyrano[3,4-c]isoquinolin-6(4H)-one-1-d (Vb-d) and 6-(difluoromethyl)-5-fluoro-1H-indole-2-carboxylic acid (VIct). LCMS m/z found 483.5 [M+H]⁺; RT=2.69 min (Method D); ¹H NMR (400 MHz, DMSO-d₆) δ 12.17 (s, 1H), 11.75 (s, 1H), 8.13 (dd, 1H), 7.70 (d, 1H), 7.54 (d, 1H), 7.45 (dd, 1H), 7.26 (d, 1H), 7.01 (s, 1H), 4.65 (d, 1H), 4.49 (d, 1H), 4.19 (d, 1H), 4.04 (d, 1H).

### (S)-2-Chloro-N-(8,9-difluoro-6-oxo-1,4,5,6-tetrahydro-2H-pyrano[3,4-c]isoquinolin-1-yl-1-d)-N-(methyl-¹³C-d₃)-4H-thieno[3,2-b]pyrrole-5-carboxamide (Compound 516)

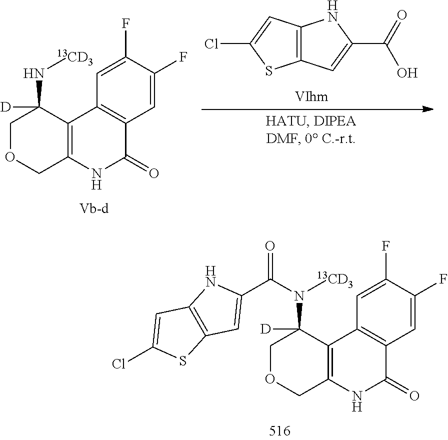

Enantiomerically pure (S)-2-chloro-N-(8,9-difluoro-6-oxo-1,4,5,6-tetrahydro-2H-pyrano[3,4-c]isoquinolin-1-yl-1-d)-N-(methyl-¹³C-d₃)-4H-thieno[3,2-b]pyrrole-5-carboxamide was synthesized in an analogous manner as described above from enantiomerically pure (S)-8,9-difluoro-1-((methyl-¹³C-d₃)amino)-1,5-dihydro-2H-pyrano[3,4-c]isoquinolin-6(4H)-one-1-d (Vb-d) and 2-chloro-4H-thieno[3,2-b]pyrrole-5-carboxylic acid (VIhm). LCMS m/z found 455.1 [M+H]⁺; RT=2.78 min (Method D); ¹H NMR (400 MHz, DMSO-d₆) δ 12.01 (s, 1H), 11.73 (s, 1H), 8.12 (dd, 1H), 7.51-7.40 (m, 1H), 7.09 (s, 1H), 6.93 (s, 1H), 4.63 (d, 1H), 4.47 (d, 1H), 4.12 (d, 1H), 4.01 (d, 1H).

### (S)-8,9-difluoroDifluorodifluoro-1-(methylamino)-1,5-dihydro-2H-pyrano[3,4-c]isoquinoline-4,6-dione hydrochloride salt (Vbiv)

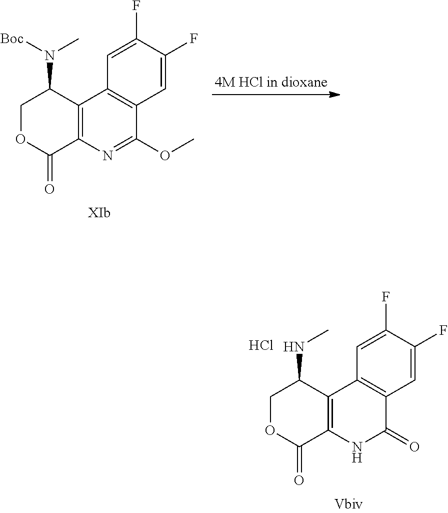 [SMILES: COc1nc2c(c3cc(F)c(F)cc13)[C@H](N(C)C(=O)OC(C)(C)C)COC2=O ; Cl ; CN[C@@H]1COC(=O)c2[nH]c(=O)c3cc(F)c(F)cc3c21]

Enantiomerically pure tert-butyl (S)-(8,9-difluoro-6-methoxy-4-oxo-1,4-dihydro-2H-pyrano[3,4-c]isoquinolin-1-yl)(methyl)carbamate (XIb) (241 mg, 0.61 mmol) and hydrogen chloride (4.0M in p-dioxane), (4.58 mL, 18.3 mmol) were stirred in p-dioxane (2 mL) at room temperature for 1 hours. Additional hydrogen chloride (4.0 M in p-dioxane) (4.58 mL, 18.3 mmol) was added and stirred at room temperature for 1 h and >70% SM was consumed. The mixture was cooled to 0-5° C. then Water (2 mL) was added dropwise and the mixture was stirred for 15 minutes and was slowly warmed to room temperature overnight. The solvent was evaporated to dryness, the residue was azeotroped with toluene (30 mL) twice and subjected to high vacuum to give crude, enantiomerically pure (S)-8,9-difluoro-1-(methylamino)-1,5-dihydro-2H-pyrano[3,4-c]isoquinoline-4,6-dione hydrochloride salt (Vbiv) (221.8 mg, >100% yield). LCMS m/z found 281.1 [M+H]⁺; RT=0.52 min (Method D); ¹H NMR (400 MHz, DMSO-d₆) δ 12.03 (s, 1H), 9.70-9.51 (m, 1H), 9.50-9.34 (m, 1H), 8.41 (dd, 1H), 8.26 (dd, 1H), 5.11-4.98 (m, 2H), 4.90 (dd, 1H), 2.72 (s, 3H).

### (S)-2-Chloro-N-(8,9-difluoro-4,6-dioxo-1,4,5,6-tetrahydro-2H-pyrano[3,4-c]isoquinolin-1-yl)-N-methyl-4H-thieno[3,2-b]pyrrole-5-carboxamide (Compound 477)

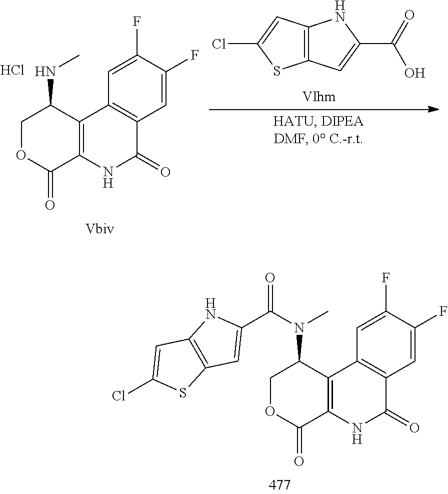

Enantiomerically pure (S)-2-chloro-N-(8,9-difluoro-4,6-dioxo-1,4,5,6-tetrahydro-2H-pyrano[3,4-c]isoquinolin-1-yl)-N-methyl-4H-thieno[3,2-b]pyrrole-5-carboxamide was synthesized in an analogous manner as described above from enantiomerically pure (S)-8,9-difluoro-1-(methylamino)-1,5-dihydro-2H-pyrano[3,4-c]isoquinoline-4,6-dione hydrochloride salt (Vbiv) and 2-chloro-4H-thieno[3,2-b]pyrrole-5-carboxylic acid (VIhm). LCMS m/z found 463.9 [M+H]⁺; RT=4.56 min (Method A); ¹H NMR (400 MHz, DMSO) δ 12.08 (s, 1H), 11.83 (s, 1H), 8.25 (dd, 1H), 7.95 (s, 1H), 7.10 (s, 1H), 6.99 (d, 1H), 6.27 (d, 1H), 5.01 (dd, 1H), 4.76 (d, 1H), 3.09 (s, 3H).

### (S)-N-(8,9-Difluoro-4,6-dioxo-1,4,5,6-tetrahydro-2H-pyrano[3,4-c]isoquinolin-1-yl)-6-(difluoromethyl)-5-fluoro-N-methyl-1H-indole-2-carboxamide (Compound 478)

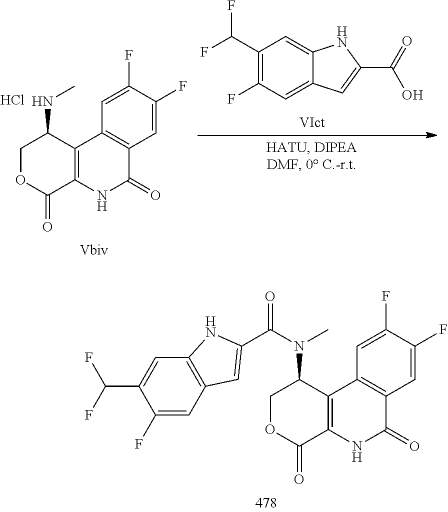

Enantiomerically pure (S)-N-(8,9-difluoro-4,6-dioxo-1,4,5,6-tetrahydro-2H-pyrano[3,4-c]isoquinolin-1-yl)-6-(difluoromethyl)-5-fluoro-N-methyl-1H-indole-2-carboxamide was synthesized in an analogous manner as described above from enantiomerically pure (S)-8,9-difluoro-1-(methylamino)-1,5-dihydro-2H-pyrano[3,4-c]isoquinoline-4,6-dione hydrochloride salt (Vbiv) and 6-(difluoromethyl)-5-fluoro-1H-indole-2-carboxylic acid (VIct). LCMS m/z found 492.2 [M+H]⁺; RT=3.16 min (Method C); ¹H NMR (400 MHz, DMSO) δ 12.21 (s, 1H), 11.86 (s, 1H), 8.26 (dd, 1H), 7.95 (s, 1H), 7.70 (d, 1H), 7.56 (d, 1H), 7.26 (d, 1H), 7.06 (d, 1H), 6.32-6.27 (m, 1H), 5.03 (dd, 1H), 4.84 (d, 1H), 3.13 (s, 3H).

### (1S)-8,9-Difluoro-4-hydroxy-1-(methylamino)-1,5-dihydro-2H-pyrano[3,4-c]isoquinolin-6(4H)-one (Vbvi)

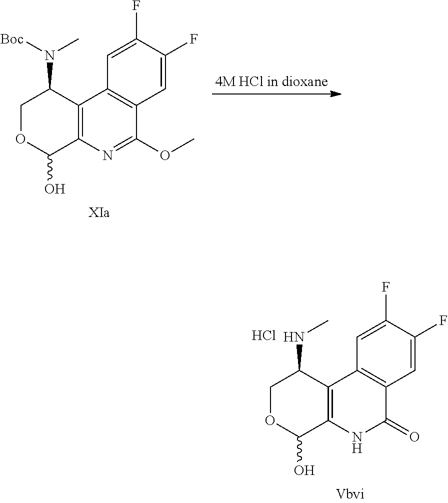 [SMILES: COc1nc2c(c3cc(F)c(F)cc13)[C@H](N(C)C(=O)OC(C)(C)C)COC2O ; Cl ; CN[C@@H]1COC(O)c2[nH]c(=O)c3cc(F)c(F)cc3c21]

To a stirred solution of tert-butyl ((1S)-8,9-difluoro-4-hydroxy-6-methoxy-1,4-dihydro-2H-pyrano[3,4-c]isoquinolin-1-yl)(methyl)carbamate (XIa) (154.9 mg, 0.39 mmol) in p-dioxane (1 mL) was added dropwise hydrogen chloride (4.0M in p-dioxane) (4.10 mL, 16.4 mmol). The mixture was stirred at room temperature for 1 hour. Significant but incomplete deprotection was observed by LC/MS. The mixture was cooled at 0-5° C. then water (0.84 mL, 46.5 mmol) was added dropwise. The reaction was stirred and slowly warmed to room temperature overnight to give a purple solution. The reaction was incomplete by LC/MS. The mixture was stirred at room temperature for an additional 24 hours. The reaction mixture was evaporated to dryness then azeotroped with toluene (2×20 mL) and the residue subjected to high vacuum for 1 h to give (1S)-8,9-difluoro-4-hydroxy-1-(methylamino)-1,5-dihydro-2H-pyrano[3,4-c]isoquinolin-6(4H)-one (Vbvi) (128.4 mg, 85% yield), as an HCl salt, as a purple solid. ¹H NMR (400 MHz, DMSO) δ 12.03-11.59 (m, 1H), 9.30-8.76 (m, 2H), 8.19-8.04 (m, 2H), 5.66-5.54 (m, 1H), 4.70-4.49 (m, 1H), 4.45-4.02 (m, 2H), 2.82-2.57 (m, 3H).

### 2-Chloro-N-((1S)-8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide (Compound 487)

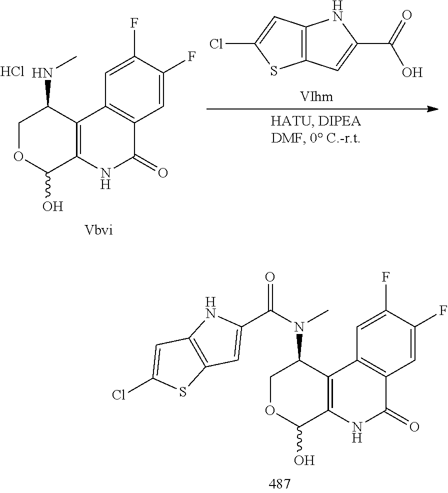

A diastereomeric mixture of 2-chloro-N-((1S)-8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide was synthesized in an analogous manner as described above from (1S)-8,9-difluoro-4-hydroxy-1-(methylamino)-1,5-dihydro-2H-pyrano[3,4-c]isoquinolin-6(4H)-one (Vbvi) and 2-chloro-3a,6a-dihydro-4H-thieno[3,2-b]pyrrole-5-carboxylic acid (VIhm). LCMS m/z found 465.9 [M+H]⁺; RT=4.31 min (Method A); ¹H NMR (400 MHz, DMSO) δ 12.00 (s, 1H), 11.83 (s, 1H), 8.13 (dd, 1H), 7.64-7.37 (m, 1H), 7.25 (d, 1H), 7.09 (s, 1H), 6.98-6.91 (m, 1H), 5.72-5.54 (m, 2H), 4.47 (d, 1H), 3.90 (d, 1H), 3.06 (s, 3H).

### N-((1S)-8,9-Difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide (Compound 488)

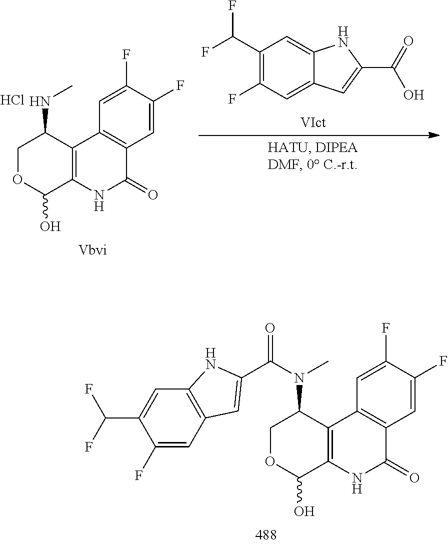

A diastereomeric mixture of N-((1S)-8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide was synthesized in an analogous manner as described above from (1S)-8,9-difluoro-4-hydroxy-1-(methylamino)-1,5-dihydro-2H-pyrano[3,4-c]isoquinolin-6(4H)-one (Vbvi) and 6-(difluoromethyl)-5-fluoro-1H-indole-2-carboxylic acid (VIct). LCMS m/z found 494.1 [M+H]⁺; RT=4.16 min (Method A); ¹H NMR (400 MHz, DMSO) δ 12.20-12.13 (m, 1H), 11.90-11.58 (m, 1H), 8.20-8.10 (m, 1H), 7.77-7.62 (m, 1H), 7.60-7.47 (m, 2H), 7.43-7.09 (m, 2H), 7.05-6.97 (m, 1H), 5.72 (d, 1H), 5.69 (d, 1H), 4.53-4.46 (m, 1H), 3.96 (d, 1H), 3.15-3.09 (m, 3H).

### (S)-5-Bromo-N-(8,9-difluoro-6-oxo-1,4,5,6-tetrahydro-2H-pyrano[3,4-c]isoquinolin-1-yl)-N-methyl-6-(trifluoromethyl)nicotinamide (Compound 365)

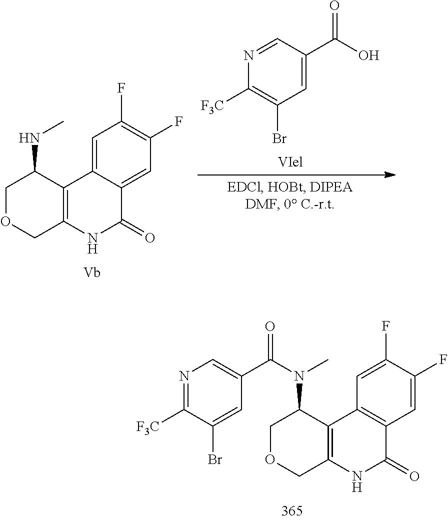

Enantiomerically pure (S)-5-bromo-N-(8,9-difluoro-6-oxo-1,4,5,6-tetrahydro-2H-pyrano[3,4-c]isoquinolin-1-yl)-N-methyl-6-(trifluoromethyl)nicotinamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-bromo-6-(trifluoromethyl)nicotinic acid (VIel). LCMS m/z found 518.0 [M+H]⁺; RT=4.47 min (Method A); ¹H NMR (400 MHz, DMSO-d₆) δ 11.70 (bs, 1H), 8.78 (s, 1H), 8.56 (s, 1H), 8.13 (t, 1H), 7.51-7.47 (m, 1H), 5.66 (s, 1H), 4.61 (d, 1H), 4.47 (d, 1H), 4.35 (d, 1H), 4.03-3.99 (m, 1H), 2.76 (s, 3H).

### 7-(Difluoromethyl)indolizine-2-carboxylic acid (VIem)

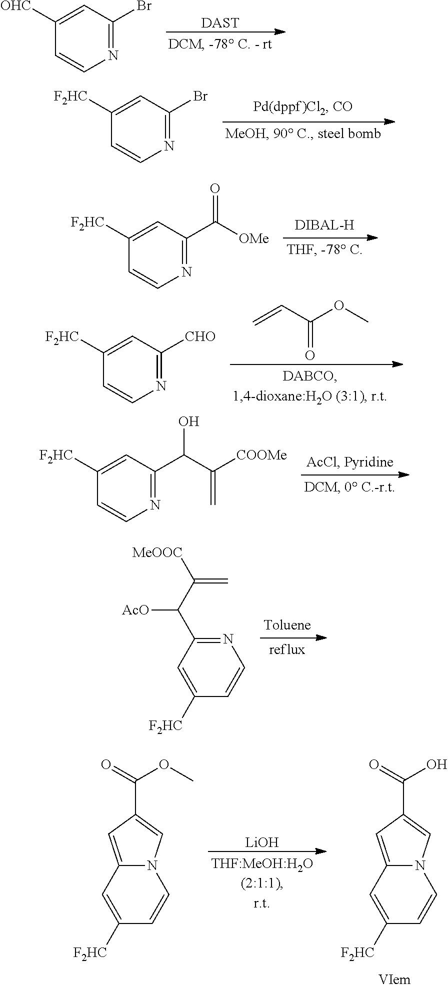

Step i. To a stirred solution of 2-bromoisonicotinaldehyde (10.0 g, 53.8 mmol) in DCM (50 mL) was added dropwise DAST (21.3 mL, 161 mmol, 3 eq.) at −78° C. then the mixture was stirred at room temperature for 5 h. The reaction mixture was poured slowly on ice and extracted with DCM (2×200 mL). Organic layer was separated, washed with water, dried over Na₂SO₄ and concentrated under reduced pressure. The crude compound was purified by column chromatography using silica gel (100-200 mesh) and 10% EtOAc in petroleum ether as eluent to afford 2-bromo-4-(difluoromethyl)pyridine (5.5 g, 49% yield) as pale-yellow liquid. ¹H NMR (400 MHz, DMSO-d₆) δ 7.23-6.96 (m, 1H), 7.66-7.64 (m, 1H), 7.86 (s, 1H), 8.59-8.58 (m, 1H).

Step ii. To a stirred solution of 2-bromo-4-(difluoromethyl)pyridine (5.0 g, 24 mmol) in MeOH (50 mL) was added Potassium Acetate (4.7 g, 48 mmol, 2 eq.) and Pd(dppf)Cl₂ (0.88 g, 1.2 mmol, 0.05 eq.) in a 250 mL steel bomb under CO atmosphere (50 psi). The reaction mixture was heated to 90° C. and stirred for 16 h. After completion of the reaction (monitored by TLC), reaction mixture was filtered through a pad of CELITE® and the filtrate was evaporated under reduced pressure. The residue was diluted with water (100 mL) and extracted with EtOAc (2×100 mL). Combined organic layer was washed with water, dried over Na₂SO₄ and concentrated under reduced pressure. The crude compound was purified by column chromatography using silica gel (100-200 mesh) and 15% EtOAc in petroleum ether as eluent to afford methyl 4-(difluoromethyl)picolinate (2.5 g, 55% yield) as a pale-yellow liquid. LCMS m/z found 187.9 [M+H]⁺; ¹H NMR (400 MHz, DMSO-d₆) δ 3.92 (s, 3H), 7.35-7.08 (m, 1H), 7.86-7.85 (m, 1H), 8.19 (s, 1H), 8.91-8.90 (m, 1H).

Step iii. To a stirred solution of methyl 4-(difluoromethyl)picolinate (2.5 g, 13.2 mmol) in THF (28 mL) was added dropwise DIBAL-H (22.6 mL, 22.6 mmol, 1.7 eq.) at −78° C. and the reaction mixture was stirred for 3 h. The reaction mixture was quenched with aq. sat. NH₄Cl solution. The reaction mixture was filtered through a pad of CELITE®, the filtrate was dried over Na₂SO₄ and concentrated under reduced pressure. Obtained crude compound was purified by column chromatography using silica gel (100-200 mesh) and 10% EtOAc in petroleum ether as eluent to afford 4-(difluoromethyl)picolinaldehyde (1.4 g, 66% yield) as pale-yellow liquid. LCMS m/z found 160.0 [M+H]⁺; RT=1.09 min (Method E).

Step iv. To a stirred solution of 4-(difluoromethyl)picolinaldehyde (1.6 g, 10.1 mmol) in 1,4-dioxane:water (3:1, 50 mL) was added methyl acrylate (1.1 mL, 12.22 mmol, 1.2 eq.) and DABCO (68 mg) at room temperature. The resulting reaction mixture was stirred at room temperature for 16 h. The reaction mixture was concentrated and diluted with water (40 mL) and extracted with EtOAc (2×70 mL). Organic layer was separated, dried over Na₂SO₄ and concentrated under reduced pressure. Obtained crude compound was purified by column chromatography using silica gel (100-200 mesh) and 10% EtOAc in petroleum ether as eluent to afford methyl 2-((4-(difluoromethyl)pyridin-2-yl)(hydroxy)methyl)acrylate (1.5 g, 60% yield) as a pale-yellow liquid. LCMS m/z found 244.0 [M+H]⁺; RT=1.16 min (Method E).

Step v. To a stirred solution of methyl 2-((4-(difluoromethyl)pyridin-2-yl)(hydroxy)methyl)acrylate (1.5 g, 6.17 mmol) in DCM (15 mL) was added pyridine (0.84 mL, 9.3 mmol, 1.5 eq.) and acetyl chloride (0.69 mL g (8.64 mmol, 1.4 eq.) at 0° C. The resulting reaction mixture was stirred at room temperature for 3 h. The reaction mixture was diluted with water (40 mL) and extracted with EtOAc (2×100 mL). Combined organic layer was washed with water (50 mL), dried over Na₂SO₄ and concentrated under reduced pressure. Obtained crude compound was diluted with toluene (15 mL) and refluxed for 16 h. The reaction mixture was concentrated under reduced pressure and purified by column chromatography using silica gel (100-200 mesh) and 10% EtOAc in petroleum ether as eluent to afford methyl 7-(difluoromethyl)indolizine-2-carboxylate (500 mg, 36% yield) as pale-yellow liquid. LCMS m/z found 226.1 [M+H]⁺; RT=1.82 min (Method E).

Step vi. To a stirred solution of methyl 7-(difluoromethyl)indolizine-2-carboxylate (500 mg, 2.22 mmol) in a mixture of THF:Water:MeOH (2:1:1, 10 mL) was added LiOH (200 mg, 11.1 mmol, 5.0 eq.) at 0° C. and the reaction was stirred at room temperature for 4 h.

The volatiles were evaporated from the reaction mixture and the residue was acidified with aq. KHSO₄ solution (10 wt %) to pH ˜2. The precipitated solids were collected by filtration, washed with water and dried under vacuum to afford 7-(difluoromethyl)indolizine-2-carboxylic acid (VIem) (300 mg, 64% yield) as a white solid. LCMS m/z found 212.4 [M+H]⁺; RT=1.40 min (Method E).

### (S)-N-(8,9-Difluoro-6-oxo-1,4,5,6-tetrahydro-2H-pyrano[3,4-c]isoquinolin-1-yl)-7-(difluoromethyl)-N-methylindolizine-2-carboxamide (Compound 374)

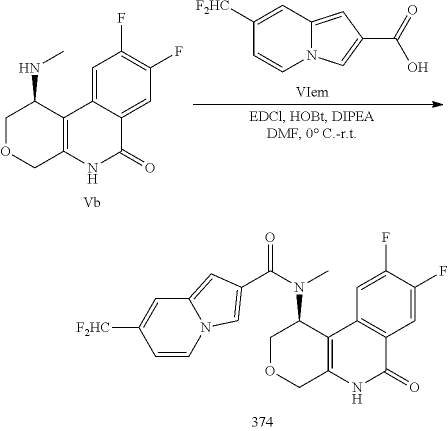 [SMILES: CN[C@@H]1COCc2[nH]c(=O)c3cc(F)c(F)cc3c21 ; O=C(O)c1cc2cc(C(F)F)ccn2c1 ; CN(C(=O)c1cc2cc(C(F)F)ccn2c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-7-(difluoromethyl)-N-methylindolizine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 7-(difluoromethyl)indolizine-2-carboxylic acid (VIem). LCMS m/z found 460.2 [M+H]+; RT=3.94 min (Method A); ¹H NMR (400 MHz, DMSO-d₆) δ 11.70 (bs, 1H), 8.36 (d, 1H), 8.15-8.06 (m, 2H), 7.72 (s, 1H), 7.47-7.43 (m, 1H), 7.08-6.73 (m, 3H), 5.72 (s, 1H), 4.61 (d, 1H), 4.47 (d, 1H), 4.17 (d, 1H), 4.04 (d, 1H), 3.02 (s, 3H).

### 6-(Difluoromethyl)indolizine-2-carboxylic acid (VIen)

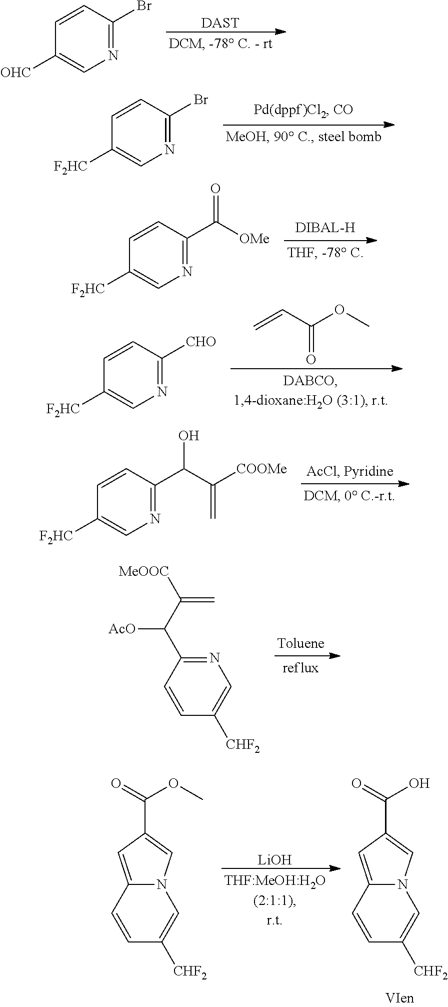

Step i. To a stirred solution of 6-bromonicotinaldehyde (10.0 g, 53.8 mmol) in DCM (50 mL) was added dropwise DAST (21.3 mL, 161 mmol, 3 eq.) at −78° C. and the mixture was stirred at room temperature for 3 h. The reaction mixture was poured slowly on ice and extracted with DCM (2×200 mL). The organic layer was separated, washed with water, dried over Na₂SO₄ and concentrated under reduced pressure. The obtained crude compound was purified by column chromatography using silica gel (100-200 mesh) and 10% EtOAc in petroleum ether as eluent to afford 2-bromo-5-(difluoromethyl)pyridine (5.5 g, 49% yield) as a pale-yellow liquid. LCMS m/z found 208.1 [M+H]⁺; RT=1.76 min (Method E).

Step ii. In a steel reaction vessel, a solution of 2-bromo-5-(difluoromethyl)pyridine (3.0 g, 14 mmol) in MeOH (30 mL) was degassed with argon gas for 20 min then of potassium acetate (2.82 g, 28.8 mmol, 2.0 eq.) and Pd(dppf)Cl₂ (527 mg, 0.72 mmol, 0.05 eq.) were added and the mixture was degassed with argon gas for 5 min. The reaction mixture was stirred under CO gas (50 psi) at 90° C. for 16 h. The reaction mixture was filtered through a pad of CELITE® and the filtrate was evaporated. Obtained crude material was purified by column chromatography using silica gel (100-200 mesh) and 10% EtOAc in petroleum ether as eluent to afford methyl 5-(difluoromethyl)picolinate (2.0 g, 74% yield) as a pale-yellow solid. LCMS m/z found 188.2 [M+H]⁺; RT=1.15 min (Method E).

Step iii. To a stirred solution of methyl 5-(difluoromethyl)picolinate (2.8 g, 15 mmol) in THF (28 mL) was added dropwise DIBAL-H (25 mL, 25 mmol, 1.7 eq.) at −78° C. and stirred for 3 h. The reaction mixture was quenched with aq. sat. NH₄Cl solution. The reaction mixture was filtered through a pad of CELITE®, the filtrate was dried over Na₂SO₄ and concentrated under reduced pressure. The crude compound was purified by column chromatography using silica gel (100-200 mesh) and 10% EtOAc in petroleum ether as eluent to afford 5-(difluoromethyl)picolinaldehyde (1.6 g, 68% yield) as a pale-yellow liquid. LCMS m/z found 160.0 [M+H]⁺; RT=1.09 min (Method E).

Step iv. To a stirred solution of 5-(difluoromethyl)picolinaldehyde (1.6 g, 10 mmol) in 1,4-dioxane:water (3:1, 50 mL) was added methyl acrylate (1.1 mL, 12 mmol, 1.2 eq.) and DABCO (68 mg) at room temperature. The resulting reaction mixture was stirred at room temperature for 16 h. The reaction mixture was concentrated and diluted with water (40 mL) and extracted with EtOAc (2×70 mL). The organic layer was separated, dried over Na₂SO₄ and concentrated under reduced pressure. The crude compound was purified by column chromatography using silica gel (100-200 mesh) and 10% EtOAc in petroleum ether as eluent to afford methyl 2-((5-(difluoromethyl)pyridin-2-yl)(hydroxy)methyl)acrylate (1.5 g, 60% yield) as a pale-yellow liquid. LCMS m/z found 244.0 [M+H]⁺; RT=1.16 min (Method E).

Step v. To a stirred solution of methyl 2-((5-(difluoromethyl)pyridin-2-yl)(hydroxy)methyl)acrylate (1.5 g, 6.2 mmol) in DCM (15 mL) was added of pyridine (0.84 mL, 9.3 mmol, 1.5 eq.) and acetyl chloride (0.69 mL, 8.64 mmol, 1.4 eq.) at 0° C. The resulting reaction mixture was stirred at room temperature for 3 h. The reaction mixture was diluted with water (40 mL) and extracted with EtOAc (2×100 mL). Combined organic layer was washed with water (50 mL), dried over Na₂SO₄ and concentrated under reduced pressure. The crude compound was diluted with toluene (15 mL) and refluxed for 16 h. The reaction mixture was concentrated under reduced pressure and purified by column chromatography using silica gel (100-200 mesh) and 10% EtOAc in petroleum ether as eluent to afford methyl 6-(difluoromethyl)indolizine-2-carboxylate (500 mg, 36% yield) as a pale-yellow liquid. LCMS m/z found 226.1 [M+H]⁺; RT=1.82 min (Method E).

Step vi. To a stirred solution of methyl 6-(difluoromethyl)indolizine-2-carboxylate (500 mg, 2.22 mmol) in a mixture of THF:Water:MeOH (2:1:1, 10 mL) was added LiOH (200 mg, 11.1 mmol, 5.0 eq.) at 0° C. and the reaction was stirred at room temperature for 4 h. The volatiles were evaporated and the residue was acidified with aq. KHSO₄ solution (10 wt % in water) to pH˜2. The precipitated solids were collected by filtration, washed with water and dried under vacuum to afford 6-(difluoromethyl)indolizine-2-carboxylic acid (VIen) (300 mg, 64% yield) as a white solid. LCMS m/z found 212.4 [M+H]⁺; RT=1.40 min (Method E).

### (S)-N-(8,9-Difluoro-6-oxo-1,4,5,6-tetrahydro-2H-pyrano[3,4-c]isoquinolin-1-yl)-6-(difluoromethyl)-N-methylindolizine-2-carboxamide (Compound 375)

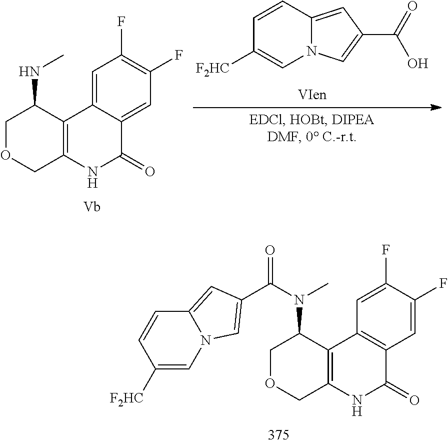 [SMILES: CN[C@@H]1COCc2[nH]c(=O)c3cc(F)c(F)cc3c21 ; O=C(O)c1cc2ccc(C(F)F)cn2c1 ; CN(C(=O)c1cc2ccc(C(F)F)cn2c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-6-(difluoromethyl)-N-methylindolizine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 6-(difluoromethyl)indolizine-2-carboxylic acid (VIen). LCMS m/z found 460.2 [M+H]⁺; RT=4.00 min (Method A); ¹H NMR (400 MHz, DMSO-d₆) δ 11.69 (bs, 1H), 8.60 (s, 1H), 8.12-8.05 (m, 2H), 7.75-7.44 (m, 2H), 7.13-6.76 (m, 3H), 5.72 (s, 1H), 4.47 (d, 1H), 4.47 (d, 1H), 4.16 (d, 1H), 4.03 (d, 1H), 3.01 (s, 3H).

### 1-(((di-tert-Butoxyphosphoryl)oxy)methyl)-5,6-difluoro-1H-indole-2-carboxylic acid (VIeo)

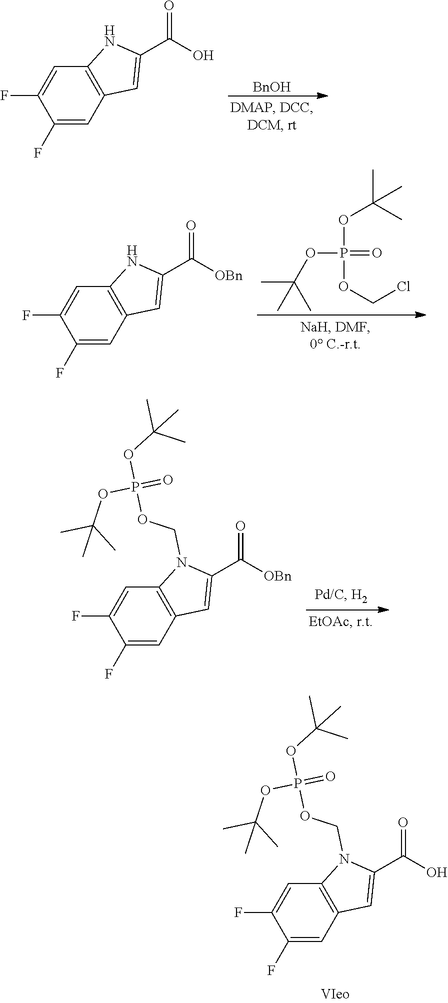

Step i. To a stirred solution of 5,6-difluoro-1H-indole-2-carboxylic acid (2.0 g, 10.15 mmol) in DCM (20 mL) at room temperature was added DCC (2.09 g, 10.2 mmol, 1 eq.) and DMAP (0.25 g, 2.0 mmol, 0.2 eq.) followed by benzyl alcohol (1.30 mL, 12.6 mmol, 1.25 eq.). The reaction mixture was stirred at room temperature for 16 h. The reaction mixture was filtered through a pad of CELITE® and partitioned between EtOAc (200 mL) and water (200 mL). The organic layer was separated, dried over Na₂SO₄ and concentrated under reduced pressure. The crude material was washed with petroleum ether to afford benzyl 5,6-difluoro-1H-indole-2-carboxylate (1.31 g, 45% yield) as an off-white solid. ¹H NMR (400 MHz, DMSO-d₆): 12.13 (s, 1H), 7.70-7.65 (m, 1H), 7.50-7.48 (m, 2H), 7.44-7.35 (m, 4H), 7.22-7.21 (m, 1H), 5.38 (s, 2H).

Step ii. To a stirred solution of benzyl 5,6-difluoro-1H-indole-2-carboxylate (1.0 g, 3.5 mmol, 1.0 eq.) in DMF (10 mL) at 0° C. was added NaH (60 wt % in mineral oil, 0.15 g, 3.8 mmol, 1.1 eq.) and stirred for 15 min. at 0° C. To the mixture was added di-tert-butyl (chloromethyl) phosphate (1.0 g, 7.0 mmol, 2.0 eq.) at 0° C. and then reaction mixture was stirred at room temperature for 16 h. The reaction mixture was added to ice-cold water and extracted with EtOAc. The organic layer was separated, dried over Na₂SO₄ and evaporated under reduced pressure. The crude material was purified by column chromatography using silica gel (100-200 mesh), 25-30% EtOAc/pet. ether as eluent to afford benzyl 1-(((di-tert-butoxyphosphoryl)oxy)methyl)-5,6-difluoro-1H-indole-2-carboxylate (0.57 g, 32% yield) as an off-white solid. ¹H NMR (400 MHz, CDCl₃): 7.43 (m, 1H), 7.39-7.34 (m, 7H), 6.42-6.40 (d, 2H), 5.36 (s, 2H), 1.51 (s, 18H).

Step iii. To a stirred solution of benzyl 1-(((di-tert-butoxyphosphoryl)oxy)methyl)-5,6-difluoro-1H-indole-2-carboxylate (700 mg, 1.37 mmol, 1.0 eq.) in EtOAc (25 mL) at room temperature was added Pd/C (10 wt % on carbon, 70 mg) and the reaction mixture was stirred at room temperature for 30 min. under hydrogen atmosphere (balloon, 1 atm). The reaction mixture was filtered through pad of CELITE® and the filtrate was evaporated under reduced pressure. The recovered solid was washed with Et₂O (15 mL) and dried under vacuum to afford 1-(((di-tert-butoxyphosphoryl)oxy)methyl)-5,6-difluoro-1H-indole-2-carboxylic acid (0.43 g, 75% yield) as a white solid. LCMS m/z found 418.4 [M−H]⁻; RT=1.99 min (Method E).

### (S)-di-tert-Butyl ((2-((8,9-difluoro-6-oxo-1,4,5,6-tetrahydro-2H-pyrano[3,4-c]isoquinolin-1-yl)(methyl)carbamoyl)-5,6-difluoro-1H-indol-1-yl)methyl)phosphate (Compound 376)

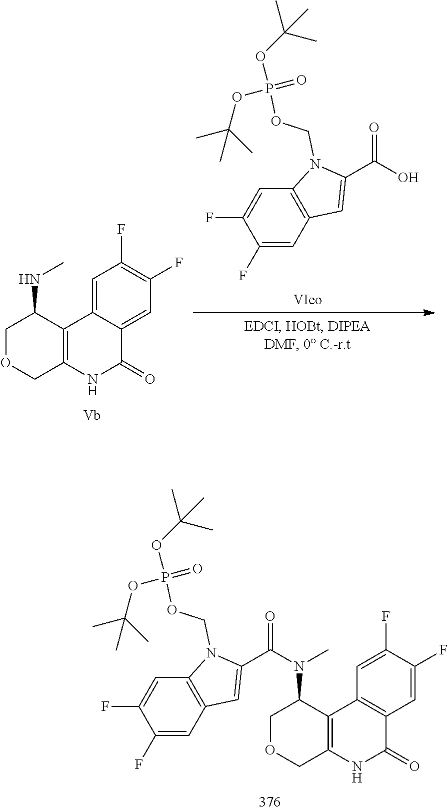 [SMILES: CN[C@@H]1COCc2[nH]c(=O)c3cc(F)c(F)cc3c21 ; CC(C)(C)OP(=O)(OCn1c(C(=O)O)cc2cc(F)c(F)cc21)OC(C)(C)C ; CN(C(=O)c1cc2cc(F)c(F)cc2n1COP(=O)(OC(C)(C)C)OC(C)(C)C)[C@@H]1COCc2[nH]c(=O)c3cc(F)c(F)cc3c21]

Enantiomerically pure (S)-di-tert-butyl ((2-((8,9-difluoro-6-oxo-1,4,5,6-tetrahydro-2H-pyrano[3,4-c]isoquinolin-1-yl)(methyl)carbamoyl)-5,6-difluoro-1H-indol-1-yl)methyl)phosphate was synthesized in an analogous manner as described above from enantiomerically pure (1S)-8,9-difluoro-1-(methylamino)-1,2,4,5-tetrahydropyrano[3,4-c]isoquinolin-6-one (Vb) and 1-(((di-tert-butoxyphosphoryl)oxy)methyl)-5,6-difluoro-1H-indole-2-carboxylic acid (VIeo). LCMS m/z found 668.2 [M+H]⁺; RT=5.41 min (Method A); ¹H NMR (400 MHz, DMSO-d₆) δ 11.7 (s, 1H), 8.13 (t, 1H), 7.79-7.74 (m, 1H), 7.65 (t, 1H), 7.54-7.49 (m, 1H), 6.91 (s, 1H), 6.23-6.08 (m, 2H), 5.68 (s, 1H), 4.62 (d, 1H), 4.47 (d, 1H), 4.31 (d, 1H), 4.04 (d, 1H), 3.0 (s, 3H), 1.24 (s, 9H), 1.14 (s, 9H).

### (S)-5-Chloro-N-(8,9-difluoro-6-oxo-1,4,5,6-tetrahydro-2H-pyrano[3,4-c]isoquinolin-1-yl)-N-methyl-6-(trifluoromethyl)nicotinamide (Compound 377)

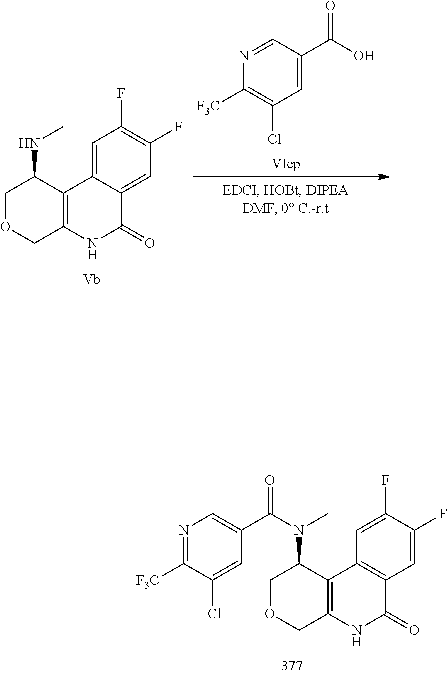

Enantiomerically pure (S)-5-chloro-N-(8,9-difluoro-6-oxo-1,4,5,6-tetrahydro-2H-pyrano[3,4-c]isoquinolin-1-yl)-N-methyl-6-(trifluoromethyl)nicotinamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-chloro-6-(trifluoromethyl)nicotinic acid (VIep). LCMS m/z found 474.1 [M+H]⁺; RT=4.41 min (Method A); ¹H NMR (400 MHz, DMSO-d₆) δ 11.69 (bs, 2H), 8.44 (bs, 1H), 8.13 (t, 1H), 7.51-7.46 (m, 1H), 5.66 (s, 1H), 4.61 (d, 1H), 4.47 (d, 1H), 4.36 (d, 1H), 4.03-3.99 (m, 1H), 2.76 (s, 3H).

### 5-Chloro-N-(8-fluoro-6-oxo-1,4,5,6-tetrahydro-2H-pyrano[3,4-c]isoquinolin-1-yl)-N-methyl-6-(trifluoromethyl)nicotinamide (Compounds 378, 379)

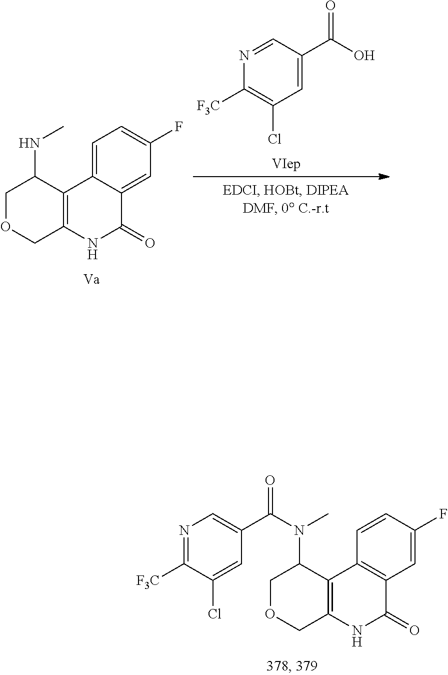

Racemic 5-chloro-N-(8-fluoro-6-oxo-1,4,5,6-tetrahydro-2H-pyrano[3,4-c]isoquinolin-1-yl)-N-methyl-6-(trifluoromethyl)nicotinamide was synthesized in an analogous manner as described above from 8-fluoro-1-(methylamino)-1,5-dihydro-2H-pyrano[3,4-c]isoquinolin-6(4H)-one (Va) and 5-chloro-6-(trifluoromethyl)nicotinic acid (VIep). The enantiomers were subsequently separated by preparative SFC: Method isocratic, Mobile phase methanol:CO₂—30:70. Column: Chiralcel OD-H (30×250 mm), 5 μm, flow rate: 100 g/min.

Enantiomer I (Compound 378): LCMS m/z found 456.1 [M+H]⁺; RT=4.17 min (Method A); ¹H NMR (400 MHz, DMSO-d₆) δ 11.61 (bs, 1H), 8.77 (s, 1H), 8.44 (s, 1H), 7.92-7.89 (m, 1H), 7.77-7.72 (m, 1H), 7.65-7.61 (m, 1H), 5.69 (s, 1H), 4.60 (d, 1H), 4.47 (d, 1H), 4.33 (d, 1H), 4.05-4.01 (m, 1H), 2.72 (s, 3H); Chiral analytical SFC: RT=1.59 min, Column: Chiralcel OJ-3 (4.6×150 mm) 3 μm, 20% (0.5% DEA in Methanol), Flow rate: 3.0 g/min.

Enantiomer II (Compound 379): LCMS m/z found 456.1 [M+H]⁺; RT=4.17 min (Method A); ¹H NMR (400 MHz, DMSO-d₆) δ 11.61 (bs, 1H), 8.77 (s, 1H), 8.44 (s, 1H), 7.92-7.89 (m, 1H), 7.77-7.72 (m, 1H), 7.65-7.61 (m, 1H), 5.69 (s, 1H), 4.60 (d, 1H), 4.47 (d, 1H), 4.33 (d, 1H), 4.05-4.01 (m, 1H), 2.72 (s, 3H); Chiral analytical SFC: RT=1.83 min, Column: Chiralcel OJ-3 (4.6×150 mm) 3 μm, 20% (0.5% DEA in Methanol), Flow rate: 3.0 g/min.

### N-(8-Fluoro-6-oxo-1,4,5,6-tetrahydro-2H-pyrano[3,4-c]isoquinolin-1-yl)-N-methyl-4H-thieno[3,2-b]pyrrole-5-carboxamide (Compounds 380, 381)

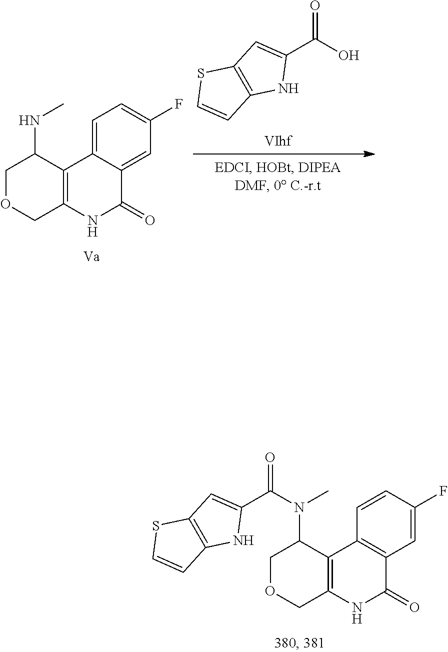

Racemic N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide was synthesized in an analogous manner as described above from 8-fluoro-1-(methylamino)-1,5-dihydro-2H-pyrano[3,4-c]isoquinolin-6(4H)-one (Va) and 4H-thieno[3,2-b]pyrrole-5-carboxylic acid (VIhf). The enantiomers were subsequently separated by preparative SFC: Method isocratic, Mobile phase methanol:CO₂—50:50. Column: Chiralpak OJ-H (30×250 mm), 5 μm, flow rate: 75 g/min.

Enantiomer I (Compound 380): LCMS m/z found 398.1 [M+H]⁺; RT=3.61 min (Method A); ¹H NMR (400 MHz, DMSO-d₆) δ 11.79 (br s, 1H), 11.62 (br s, 1H), 7.90-7.88 (m, 1H), 7.61 (t, 1H), 7.52 (bs, 1H), 7.42 (d, 1H), 7.00 (d, 1H), 6.91 (bs, 1H), 5.73 (bs, 1H), 4.62 (d, 1H), 4.47 (d, 1H), 4.13-4.02 (m, 2H), 3.11 (s, 3H); Chiral analytical SFC: RT=2.55 min, Column: Chiralpak OJ-3 (4.6×150 mm) 3 μm, 40% Methanol, Flow rate: 3.0 g/min.

Enantiomer II (Compound 381): LCMS m/z found 398.1 [M+H]⁺; RT=3.61 min (Method A); ¹H NMR (400 MHz, DMSO-d₆) δ 11.79 (br s, 1H), 11.62 (br s, 1H), 7.90-7.88 (m, 1H), 7.61 (t, 1H), 7.52 (bs, 1H), 7.42 (d, 1H), 7.00 (d, 1H), 6.91 (bs, 1H), 5.73 (bs, 1H), 4.62 (d, 1H), 4.47 (d, 1H), 4.13-4.02 (m, 2H), 3.11 (s, 3H); Chiral analytical SFC: RT=4.68 min, Column: Chiralpak OJ-3 (4.6×150 mm) 3 μm, 40% Methanol, Flow rate: 3.0 g/min.

### 4-Cyano-6-fluoro-N-(8-fluoro-6-oxo-1,4,5,6-tetrahydro-2H-pyrano[3,4-c]isoquinolin-1-yl)-N-methyl-1H-indole-2-carboxamide (Compounds 382, 383)

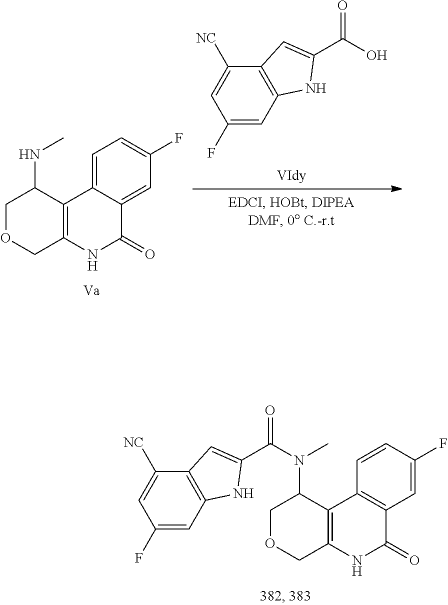

Racemic 4-cyano-6-fluoro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from 8-fluoro-1-(methylamino)-1,5-dihydro-2H-pyrano[3,4-c]isoquinolin-6(4H)-one (Va) and 4-cyano-6-fluoro-1H-indole-2-carboxylic acid (VIdy). The enantiomers were subsequently separated by preparative SFC: Method isocratic, Mobile phase methanol:CO₂—40:60. Column: Chiralpak OJ-H (30×250 mm), 5 μm, flow rate: 100 g/min.

Enantiomer I (Compound 382): LCMS m/z found 435.2 [M+H]⁺; RT=3.88 min (Method A); ¹H NMR (400 MHz, DMSO-d₆) δ 12.41 (br s, 1H), 11.64 (bs, 1H), 7.91-7.37 (m, 5H), 6.97 (s, 1H), 5.75 (s, 1H), 4.63 (d, 1H), 4.48 (d, 1H), 4.19 (d, 1H), 4.06 (d, 1H), 3.17 (s, 3H); Chiral analytical SFC: RT=2.60 min, Column: Chiralpak IG-3 (4.6×150 mm) 3 m, 40% Methanol, Flow rate: 3.0 g/min.

Enantiomer II (Compound 383): LCMS m/z found 435.2 [M+H]⁺; RT=3.88 min (Method A); ¹H NMR (400 MHz, DMSO-d₆) δ 12.41 (br s, 1H), 11.64 (bs, 1H), 7.91-7.37 (m, 5H), 6.97 (s, 1H), 5.75 (s, 1H), 4.63 (d, 1H), 4.48 (d, 1H), 4.19 (d, 1H), 4.06 (d, 1H), 3.17 (s, 3H); Chiral analytical SFC: RT=6.16 min, Column: Chiralpak IG-3 (4.6×150 mm) 3 m, 40% Methanol, Flow rate: 3.0 g/min.

### N-(8,9-Difluoro-6-oxo-1,2,3,4,5,6-hexahydrobenzo[c][1,7]naphthyridin-1-yl)-7-(difluoromethyl)-N-methylindolizine-2-carboxamide (Compounds 385, 386)

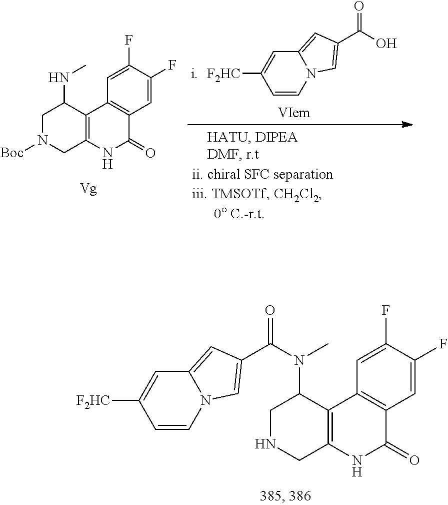 [SMILES: CNC1CN(C(=O)OC(C)(C)C)Cc2[nH]c(=O)c3cc(F)c(F)cc3c21 ; O=C(O)c1cc2cc(C(F)F)ccn2c1 ; CN(C(=O)c1cc2cc(C(F)F)ccn2c1)C1CNCc2[nH]c(=O)c3cc(F)c(F)cc3c21]

Racemic tert-butyl 1-(7-(difluoromethyl)-N-methylindolizine-2-carboxamido)-8,9-difluoro-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 7-(difluoromethyl)indolizine-2-carboxylic acid (VIem). The enantiomers were subsequently separated by preparative SFC: Method isocratic, Mobile phase methanol:CO₂—40:60. Column: Chiralcel IA-3 (30×250 mm), 5 μm, flow rate: 110 g/min, and each was converted to the final product in an analogous manner as described above (for Compounds 160, 161).

Enantiomer I (Compound 385): LCMS m/z found 459.2 [M+H]⁺; RT=3.20 min (Method A); ¹H NMR (400 MHz, DMSO-d₆) δ 11.62 (bs, 1H), 8.36 (d, 1H), 8.10 (t, 1H), 8.04 (s,(,(, 1H), 7.71 (s, 1H), 7.33-7.28 (m, 1H), 7.08-6.72 (m, 3H), 5.64 (s, 1H), 3.78 (d, 1H), 3.64 (d, 1H), 3.16 (bs, 2H), 3.03-2.76 (m, 4H); Chiral analytical SFC: RT=1.75 min, Column: Chiralpak AD-3 (4.6×150 mm) 3 μm, 40% (0.5% DEA in Methanol), Flow rate: 3.0 g/min.

Enantiomer II (Compound 386): LCMS m/z found 459.2 [M+H]⁺; RT=3.20 min (Method A); ¹H NMR (400 MHz, DMSO-d₆) δ 11.62 (bs, 1H), 8.36 (d, 1H), 8.10 (t, 1H), 8.04 (s,(,(, 1H), 7.71 (s, 1H), 7.33-7.28 (m, 1H), 7.08-6.72 (m, 3H), 5.64 (s, 1H), 3.78 (d, 1H), 3.64 (d, 1H), 3.16 (bs, 2H), 3.03-2.76 (m, 4H); Chiral analytical SFC: RT=4.20 min, Column: Chiralpak AD-3 (4.6×150 mm) 3 μm, 40% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-6-(difluoromethyl)-N-methylindolizine-2-carboxamide (Compounds 387, 388)

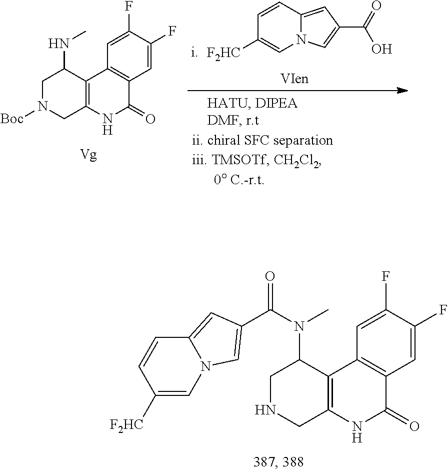 [SMILES: CNC1CN(C(=O)OC(C)(C)C)Cc2[nH]c(=O)c3cc(F)c(F)cc3c21 ; O=C(O)c1cc2ccc(C(F)F)cn2c1 ; CN(C(=O)c1cc2ccc(C(F)F)cn2c1)C1CNCc2[nH]c(=O)c3cc(F)c(F)cc3c21]

Racemic tert-butyl 1-(6-(difluoromethyl)-N-methylindolizine-2-carboxamido)-8,9-difluoro-6-oxo-1,4,5,6-tetrahydrobenzo[c][1,7]naphthyridine-3(2H)-carboxylate was synthesized in an analogous manner as described above tert-butyl 8,9-difluoro-1-(methylamino)-6-oxo-1,4,5,6-tetrahydrobenzo[c][1,7]naphthyridine-3(2H)-carboxylate (Vg) and 6-(difluoromethyl)indolizine-2-carboxylic acid (VIen). The enantiomers were subsequently separated by preparative SFC: Method isocratic, Mobile phase methanol:CO₂—40:60. Column: Chiralcel OX-H (30×250 mm), 5μ, flow rate: 110 g/min, and each was converted to the final product in an analogous manner as described above (for Compounds 160, 161).

Enantiomer I (Compound 387): LCMS m/z found 459.2 [M+H]⁺; RT=3.28 min (Method A); ¹H NMR (400 MHz, DMSO-d₆) δ 11.7177 (bs, 1H), 8.61 (bs, 1H), 8.13 (t, 1H), 8.04 (s, 1H), 7.57 (d, 1H), 7.33-7.28 (m, 1H), 7.13-6.84 (m, 2H), 6.74 (s, 1H), 5.75 (s, 1H), 3.93-3.80 (m, 2H), 3.2 (bs, 2H), 2.96-2.73 (m, 4H). Chiral analytical SFC: RT=1.63 min, Column: Chiralpak AD-3 (4.6×150 mm) 3 μm, 40% (0.5% DEA in Methanol), Flow rate: 3.0 g/min.

Enantiomer II (Compound 388): LCMS m/z found 459.2 [M+H]⁺; RT=3.28 min (Method A); ¹H NMR (400 MHz, DMSO-d₆) δ 11.7177 (bs, 1H), 8.61 (bs, 1H), 8.13 (t, 1H), 8.04 (s, 1H), 7.57 (d, 1H), 7.33-7.28 (m, 1H), 7.13-6.84 (m, 2H), 6.74 (s, 1H), 5.75 (s, 1H), 3.93-3.80 (m, 2H), 3.2 (bs, 2H), 2.96-2.73 (m, 4HH). Chiral analytical SFC: RT=3.85 min, Column: Chiralpak AD-3 (4.6×150 mm) 3 μm, 40% (0.5% DEA in Methanol), Flow rate: 3.0 g/min.

### N-(8-Fluoro-6-oxo-1,4,5,6-tetrahydro-2H-pyrano[3,4-c]isoquinolin-1-yl)-N-methylindolizine-6-carboxamide (Compounds 389, 390)

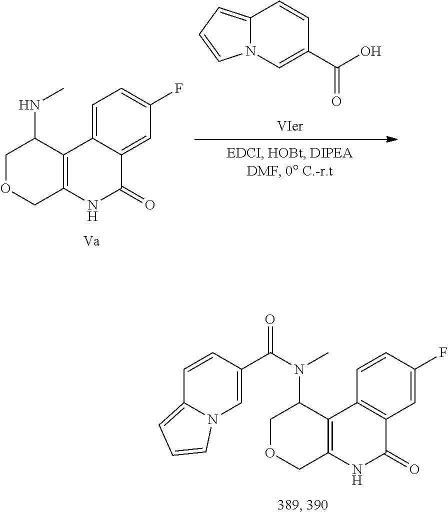 [SMILES: CNC1COCc2[nH]c(=O)c3cc(F)ccc3c21 ; O=C(O)c1ccc2cccn2c1 ; CN(C(=O)c1ccc2cccn2c1)C1COCc2[nH]c(=O)c3cc(F)ccc3c21]

Racemic N-(8-fluoro-6-oxo-1,4,5,6-tetrahydro-2H-pyrano[3,4-c]isoquinolin-1-yl)-N-methylindolizine-6-carboxamide was synthesized in an analogous manner as described above from 8-fluoro-1-(methylamino)-1,5-dihydro-2H-pyrano[3,4-c]isoquinolin-6(4H)-one (Va) and indolizine-6-carboxylic acid (VIer). The enantiomers were subsequently separated by preparative SFC: Method isocratic, Mobile phase methanol:CO₂—40:60. Column: Chiralcel OX-H (30×250 mm), 5 μm, flow rate: 110 g/min.

Enantiomer I (Compound 389): LCMS m/z found 392.2 [M+H]⁺; RT=3.63 min (Method A); ¹H NMR (400 MHz, DMSO-d₆) δ 11.60 (bs, 1H), 8.52 (bs, 1H), 7.91 (d, 1H), 7.74 (t, 1H), 7.62 (bs, 2H), 7.44 (d, 1H), 6.81 (bs, 1H), 6.67 (d, 1H), 6.45 (bs, 1H), 5.66 (s, 1H), 4.59 (d, 1H), 4.47 (d, 1H), 4.22 (d, 1H), 4.04 (d, 1H), 2.84 (s, 3H); Chiral analytical SFC: RT=2.27 min, Column: Chiralpak IA-3 (4.6×150 mm) 3 μm, 40% (Acetonitrile:Methanol)(1:1), Flow rate: 3.0 g/min.

Enantiomer II (Compound 390): LCMS m/z found 392.2 [M+H]⁺; RT=3.63 min (Method A); ¹H NMR (400 MHz, DMSO-d₆) δ 11.60 (bs, 1H), 8.52 (bs, 1H), 7.91 (d, 1H), 7.74 (t, 1H), 7.62 (bs, 2H), 7.44 (d, 1H), 6.81 (bs, 1H), 6.67 (d, 1H), 6.45 (bs, 1H), 5.66 (s, 1H), 4.59 (d, 1H), 4.47 (d, 1H), 4.22 (d, 1H), 4.04 (d, 1H), 2.84 (s, 3H); Chiral analytical SFC: RT=3.94 min, Column: Chiralpak IA-3 (4.6×150 mm) 3 μm, 40% (Acetonitrile:Methanol)(1:1), Flow rate: 3.0 g/min.

### N-(8,9-difluoro-6-oxo-1,2,3,4,5,6-hexahydrobenzo[c][1,7]naphthyridin-1-yl)-N-methylindolizine-6-carboxamide (Compounds 391, 392)

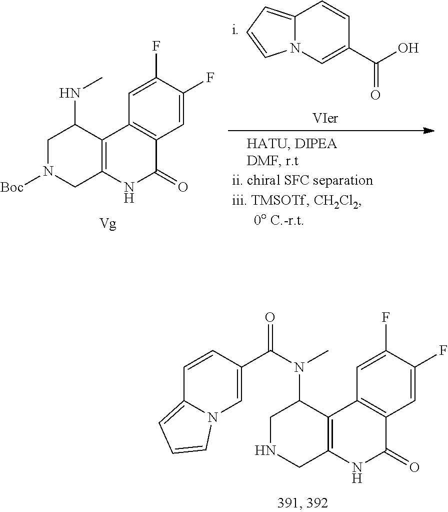 [SMILES: CNC1CN(C(=O)OC(C)(C)C)Cc2[nH]c(=O)c3cc(F)c(F)cc3c21 ; O=C(O)c1ccc2cccn2c1 ; CN(C(=O)c1ccc2cccn2c1)C1CNCc2[nH]c(=O)c3cc(F)c(F)cc3c21]

Racemic tert-butyl 8,9-difluoro-1-(N-methylindolizine-6-carboxamido)-6-oxo-1,4,5,6-tetrahydrobenzo[c][1,7]naphthyridine-3(2H)-carboxylate was synthesized in an analogous manner as described above tert-butyl 8,9-difluoro-1-(methylamino)-6-oxo-1,4,5,6-tetrahydrobenzo[c][1,7]naphthyridine-3(2H)-carboxylate (Vg) and indolizine-6-carboxylic acid (VIer). The enantiomers were subsequently separated by preparative SFC: Method isocratic, Mobile phase methanol:CO₂—40:60. Column: Cellulose-2 (30×250 mm), 5 μm, flow rate: 110 g/min.

Enantiomer I (Compound 391): LCMS m/z found 409.2 [M+H]⁺; RT=3.10 min (Method A); ¹H NMR (400 MHz, DMSO-d₆) δ 11.62 (bs, 1H), 8.52 (bs, 1H), 8.12 (t, 1H), 7.63 (s, 1H), 7.46-7.34 (m, 2H), 6.81 (bs, 1H), 6.66 (d, 1H), 6.45 (bs, 1H), 5.56 (s, 1H), 3.77 (d, 1H), 3.64 (d, 1H), 3.22-3.13 (m, 2H), 2.85-2.65 (m, 4H); Chiral analytical SFC: RT=2.07 min, Column: Chiralpak AD-3 (4.6×150 mm) 3 μm, 40% (0.5% DEA in Methanol), Flow rate: 3.0 g/min, and each was converted to the final product in an analogous manner as described above (for Compounds 160, 161).

Enantiomer II (Compound 392): LCMS m/z found 409.2 [M+H]⁺; RT=3.10 min (Method A); ¹H NMR (400 MHz, DMSO-d₆) δ 11.62 (bs, 1H), 8.52 (bs, 1H), 8.12 (t, 1H), 7.63 (s, 1H), 7.46-7.34 (m, 2H), 6.81 (bs, 1H), 6.66 (d, 1H), 6.45 (bs, 1H), 5.56 (s, 1H), 3.77 (d, 1H), 3.64 (d, 1H), 3.22-3.13 (m, 2H), 2.85-2.65 (m, 4H); Chiral analytical SFC: RT=5.37 min, Column: Chiralpak AD-3 (4.6×150 mm) 3 μm, 40% (0.5% DEA in Methanol), Flow rate: 3.0 g/min.

### 4-Ethyl-6-fluoro-N-(8-fluoro-6-oxo-1,4,5,6-tetrahydro-2H-pyrano[3,4-c]isoquinolin-1-yl)-N-methyl-1H-indole-2-carboxamide (Compound 393, 394)

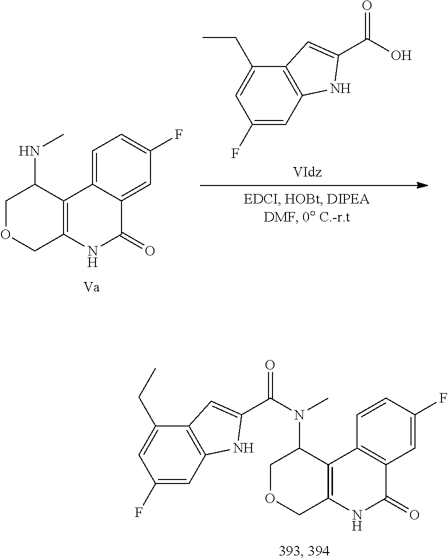 [SMILES: CNC1COCc2[nH]c(=O)c3cc(F)ccc3c21 ; CCc1cc(F)cc2[nH]c(C(=O)O)cc12 ; CCc1cc(F)cc2[nH]c(C(=O)N(C)C3COCc4[nH]c(=O)c5cc(F)ccc5c43)cc12]

Racemic 4-ethyl-6-fluoro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from 8-fluoro-1-(methylamino)-1,5-dihydro-2H-pyrano[3,4-c]isoquinolin-6(4H)-one (Va) and 4-ethyl-6-fluoro-1H-indole-2-carboxylic acid (VIdz). The enantiomers were subsequently separated by preparative SFC: Method isocratic, Mobile phase methanol:CO₂—40:60. Column: Chiralpak IA-3 (30×250 mm), 5 μm, flow rate: 110 g/min.

Enantiomer I (Compound 393): LCMS m/z found 438.2 [M+H]⁺; RT=4.62 min (Method A); ¹H NMR (400 MHz, DMSO-d₆) δ 646411.72 (bs, 1H), 11.64 (bs, 1H), 7.9 (d, 1H), 7.63 (t, 1H), 7.54 (bs, 1H), 6.99 (t, 2H), 6.75 (d, 1H), 5.77 (s, 1H), 4.64 (d, 1H), 4.49 (d, 1H), 4.15 (d, 1H), 4.05 (d, 1H), 3.16 (s, 3H), 2.87-2.80 (m, 2H), 1.23 (t, 3H); Chiral analytical SFC: RT=8.83 min, Column: Chiralpak AD-3 (4.6×150 mm) 3 μm, 40% (0.5% DEA in Methanol), Flow rate: 3.0 g/min.

Enantiomer II (Compound 394): LCMS m/z found 438.2 [M+H]⁺; RT=4.62 min (Method A); ¹H NMR (400 MHz, DMSO-d₆) δ 11.72 (bs, 1H), 11.64 (bs, 1H), 7.9 (d, 1H), 7.63 (t, 1H), 7.54 (bs, 1H), 6.99 (t, 2H), 6.75 (d, 1H), 5.77 (s, 1H), 4.64 (d, 1H), 4.49 (d, 1H), 4.15 (d, 1H), 4.05 (d, 1H), 3.16 (s, 3H), 2.87-2.80 (m, 2H), 1.23 (t, 3H); Chiral analytical SFC: RT=10.50 min, Column: Chiralpak AD-3 (4.6×150 mm) 3 μm, 40% (0.5% DEA in Methanol), Flow rate: 3.0 g/min.

### N-(8-Fluoro-6-oxo-1,4,5,6-tetrahydro-2H-pyrano[3,4-c]isoquinolin-1-yl)-N-methyl-5-(methylsulfonyl)-1H-indole-2-carboxamide (Compounds 395, 396)

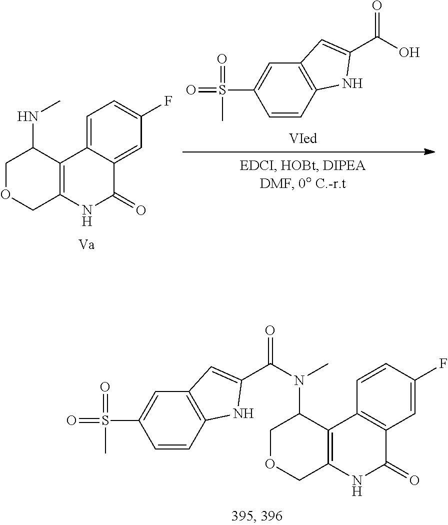

Racemic N-(8-fluoro-6-oxo-1,4,5,6-tetrahydro-2H-pyrano[3,4-c]isoquinolin-1-yl)-N-methyl-5-(methylsulfonyl)-1H-indole-2-carboxamide was synthesized in an analogous manner as described above from 8-fluoro-1-(methylamino)-1,5-dihydro-2H-pyrano[3,4-c]isoquinolin-6(4H)-one (Va) and 5-(methylsulfonyl)-1H-indole-2-carboxylic acid (VIed).

The enantiomers were subsequently separated by preparative SFC: Method isocratic, Mobile phase methanol:CO₂—40:60. Column: Chiralpak IA-3 (30×250 mm), 5 μm, flow rate: 110 g/min.

Enantiomer I (Compound 395): LCMS m/z found 470.2 [M+H]⁺; RT=2.80 min (Method A); ¹H NMR (400 MHz, DMSO-d₆) δ 12.20 (bs, 1H), 11.70 (bs, 1H), 8.23 (bs, 1H), 7.91 (d, 1H), 7.72 (d, 1H), 7.67-7.62 (m, 2H), 7.55-7.52 (m, 1H), 7.14 (s, 1H), 5.76 (s, 1H), 4.64 (d, 1H), 4.49 (d, 1H), 4.19 (d, 1H), 4.07 (d, 1H), 3.16 (s, 3H), 3.15 (s, 3H); Chiral analytical SFC: RT=5.36 min, Column: Chiralpak IA-3 (4.6×150 mm) 3 μm, 40% (Acetonitrile: Methanol)(1:1), Flow rate: 3.0 g/min.

Enantiomer II (Compound 396): LCMS m/z found 470.2 [M+H]⁺; RT=2.80 min (Method A); ¹H NMR (400 MHz, DMSO-d₆) δ 12.20 (bs, 1H), 11.70 (bs, 1H), 8.23 (bs, 1H), 7.91 (d, 1H), 7.72 (d, 1H), 7.67-7.62 (m, 2H), 7.55-7.52 (m, 1H), 7.14 (s, 1H), 5.76 (s, 1H), 4.64 (d, 1H), 4.49 (d, 1H), 4.19 (d, 1H), 4.07 (d, 1H), 3.16 (s, 3H), 3.15 (s, 3H); Chiral analytical SFC: RT=8.69 min, Column: Chiralpak IA-3 (4.6×150 mm) 3 μm, 40% (Acetonitrile: Methanol)(1:1), Flow rate: 3.0 g/min.

### 5-Cyano-N-(8-fluoro-6-oxo-1,4,5,6-tetrahydro-2H-pyrano[3,4-c]isoquinolin-1-yl)-N-methyl-1H-indole-2-carboxamide (Compounds 397, 398)

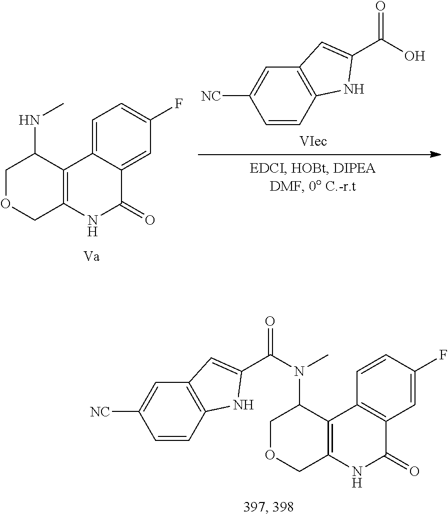

Racemic 5-cyano-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from 8-fluoro-1-(methylamino)-1,5-dihydro-2H-pyrano[3,4-c]isoquinolin-6(4H)-one (Va) and 5-cyano-1H-indole-2-carboxylic acid (VIec). The enantiomers were subsequently separated by preparative SFC: Method isocratic, Mobile phase methanol:CO₂—40:60. Column: Chiralcel OJ-3 (30×250 mm) 5 μm, flow rate: 110 g/min.

Enantiomer I (Compound 397): LCMS m/z found 417.1 [M+H]⁺; RT=3.47 min (Method A); ¹H NMR (400 MHz, DMSO-d₆) δ 12.27 (bs, 1H), 11.64 (bs, 1H), 8.17 (bs, 1H), 7.91 (d, 1H), 7.66-7.50 (m, 4H), 7.04 (s, 1H), 5.75 (s, 1H), 4.64 (d, 1H), 4.49 (d, 1H), 4.17 (d, 1H), 4.06 (d, 1H), 3.14 (s, 3H); Chiral analytical SFC: RT=1.77 min, Column: Chiralpak OJ-3 (4.6×150 mm) 3 μm, 40% Methanol, Flow rate: 3.0 g/min.

Enantiomer II (Compound 398): LCMS m/z found 417.1 [M+H]⁺; RT=3.47 min (Method A); ¹H NMR (400 MHz, DMSO-d₆) δ 12.27 (bs, 1H), 11.64 (bs, 1H), 8.17 (bs, 1H), 7.91 (d, 1H), 7.66-7.50 (m, 4H), 7.04 (s, 1H), 5.75 (s, 1H), 4.64 (d, 1H), 4.49 (d, 1H), 4.17 (d, 1H), 4.06 (d, 1H), 3.14 (s, 3H); Chiral analytical SFC: RT=2.34 min, Column: Chiralpak OJ-3 (4.6×150 mm) 3 μm, 40% Methanol, Flow rate: 3.0 g/min.

### 4-Cyano-N-(8,9-difluoro-6-oxo-1,2,3,4,5,6-hexahydrobenzo[c][1,7]naphthyridin-1-yl)-6-fluoro-N-methyl-1H-indole-2-carboxamide (Compounds 399, 400)

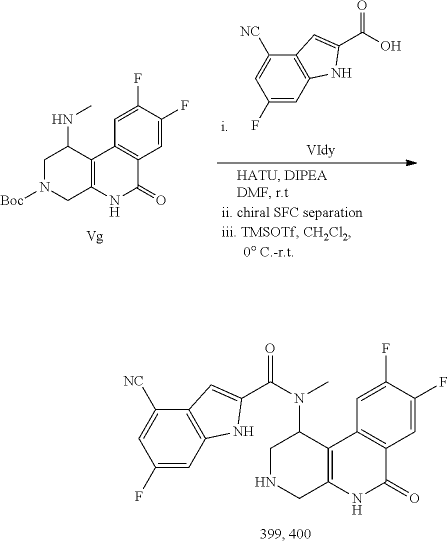 [SMILES: CNC1CN(C(=O)OC(C)(C)C)Cc2[nH]c(=O)c3cc(F)c(F)cc3c21 ; N#Cc1cc(F)cc2[nH]c(C(=O)O)cc12 ; CN(C(=O)c1cc2c(C#N)cc(F)cc2[nH]1)C1CNCc2[nH]c(=O)c3cc(F)c(F)cc3c21]

Racemic tert-butyl 1-(4-cyano-6-fluoro-N-methyl-1H-indole-2-carboxamido)-8,9-difluoro-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 4-cyano-6-fluoro-1H-indole-2-carboxylic acid (VIdy). The enantiomers were subsequently separated by preparative SFC: Method isocratic, Mobile phase methanol:CO₂—25:75. Column: Chiralpak IA-3 (30×250 mm) 5 μm, flow rate: 110 g/min, and each was converted to the final product in an analogous manner as described above (for Compounds 160, 161).

Enantiomer I (Compound 399): LCMS m/z found 452.2 [M+H]⁺; RT=3.34 min (Method A); ¹H NMR (400 MHz, DMSO-d₆) δ 12.44 (bs, 1H), 11.65 (bs, 1H), 8.11 (t, 1H), 7.67-7.57 (m, 2H), 7.28 (t, 1H), 7.01 (s, 1H), 5.66 (s, 1H), 3.81 (d, 1H), 3.62 (d, 1H), 3.19-3.16 (m, 6H). Chiral analytical SFC: RT=2.34 min, Column: Chiralpak AD-3 (4.6×150 mm) 3 μm, 40% Methanol, Flow rate: 3.0 g/min.

Enantiomer II (Compound 400): LCMS m/z found 452.2 [M+H]⁺; RT=3.34 min (Method A); ¹H NMR (400 MHz, DMSO-d₆) δ 12.44 (bs, 1H), 11.65 (bs, 1H), 8.11 (t, 1H), 7.67-7.57 (m, 2H), 7.28 (t, 1H), 7.01 (s, 1H), 5.66 (s, 1H), 3.81 (d, 1H), 3.62 (d, 1H), 3.19-3.16 (m, 6H). Chiral analytical SFC: RT=3.45 min, Column: Chiralpak AD-3 (4.6×150 mm) 3 μm, 40% Methanol, Flow rate: 3.0 g/min.

### N-(8,9-Difluoro-6-oxo-1,2,3,4,5,6-hexahydrobenzo[c][1,7]naphthyridin-1-yl)-4-ethyl-6-fluoro-N-methyl-1H-indole-2-carboxamide (Compounds 401, 402)

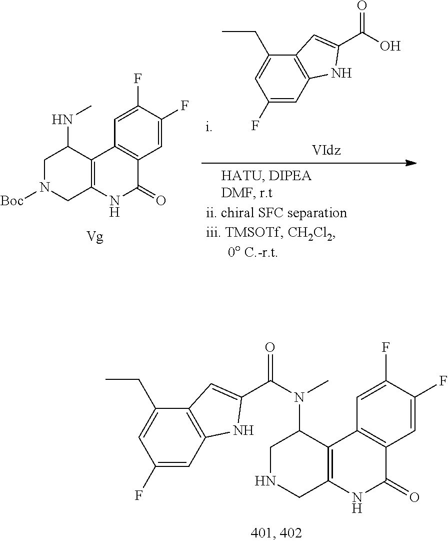 [SMILES: CNC1CN(C(=O)OC(C)(C)C)Cc2[nH]c(=O)c3cc(F)c(F)cc3c21 ; CCc1cc(F)cc2[nH]c(C(=O)O)cc12 ; CCc1cc(F)cc2[nH]c(C(=O)N(C)C3CNCc4[nH]c(=O)c5cc(F)c(F)cc5c43)cc12]

Racemic tert-butyl 1-(4-ethyl-6-fluoro-N-methyl-1H-indole-2-carboxamido)-8,9-difluoro-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 4-ethyl-6-fluoro-1H-indole-2-carboxylic acid (VIdz). The enantiomers were subsequently separated by preparative SFC: Method isocratic, Mobile phase methanol:CO₂—25:75. Column: Chiralpak IA-3 (30×250 mm), 5 μm, flow rate: 110 g/min, and each was converted to the final product in an analogous manner as described above (for Compounds 160, 161).

Enantiomer I (Compound 401): LCMS m/z found 455.2 [M+H]⁺; RT=3.96 min (Method A); ¹H NMR (400 MHz, DMSO-d₆) δ 11.74 (bs, 1H), 11.64 (bs, 1H), 8.10 (bs, 1H), 7.32-7.27 (m, 1H), 7.01-6.97 (m, 2H), 6.75 (d, 1H), 5.69 (s, 1H), 3.81 (d, 1H), 3.65 (d, 1H), 3.21-2.78 (m, 8H), 1.23 (t, 3H); Chiral analytical SFC: RT=2.53 min, Column: Chiralpak OJ-3 (4.6×150 mm) 3 μm, 20% (0.5% DEA in Methanol), Flow rate: 3.0 g/min.

Enantiomer II (Compound 402): LCMS m/z found 455.2 [M+H]⁺; RT=3.96 min (Method A); ¹H NMR (400 MHz, DMSO-d₆) δ 11.74 (bs, 1H), 11.64 (bs, 1H), 8.10 (bs, 1H), 7.32-7.27 (m, 1H), 7.01-6.97 (m, 2H), 6.75 (d, 1H), 5.69 (s, 1H), 3.81 (d, 1H), 3.65 (d, 1H), 3.21-2.78 (m, 8H), 1.23 (t, 3H); Chiral analytical SFC: RT=4.82 min, Column: Chiralpak OJ-3 (4.6×150 mm) 3 μm, 20% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-N-methyl-5-(methylsulfonyl)-1H-indole-2-carboxamide (Compounds 403, 404)

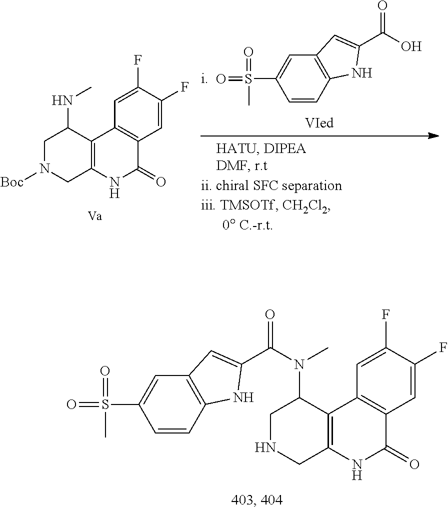

Racemic tert-butyl 8,9-difluoro-1-(N-methyl-5-(methylsulfonyl)-1H-indole-2-carboxamido)-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 5-(methylsulfonyl)-1H-indole-2-carboxylic acid (VIed). The enantiomers were subsequently separated by preparative SFC: Method isocratic, Mobile phase methanol:CO₂—40:60. Column: Chiralpak IC-3 (30×250 mm), 5 μm, flow rate: 110 g/min, and each was converted to the final product in an analogous manner as described above (for Compounds 160, 161).

Enantiomer I (Compound 403): LCMS m/z found 487.1 [M+H]⁺; RT=2.37 min (Method A); ¹H NMR (400 MHz, DMSO-d₆) δ 12.27 (bs, 1H), 11.65 (bs, 1H), 8.23 (bs, 1H), 8.11 (t, 1H), 7.73-7.65 (m, 2H), 7.31-7.26 (m, 1H), 7.14 (bs, 1H), 5.68 (s, 1H), 3.81 (d, 1H), 3.66 (d, 1H), 3.16-2.78 (m, 9H); Chiral analytical SFC: RT=4.04 min, Column: Chiralpak OD-3 (4.6×150 mm) 3 μm, 30% (0.5% DEA in Methanol), Flow rate: 3.0 g/min.

Enantiomer II (Compound 404): LCMS m/z found 487.1 [M+H]⁺; RT=2.37 min (Method A); ¹H NMR (400 MHz, DMSO-d₆) δ 12.27 (bs, 1H), 11.65 (bs, 1H), 8.23 (bs, 1H), 8.11 (t, 1H), 7.73-7.65 (m, 2H), 7.31-7.26 (m, 1H), 7.14 (bs, 1H), 5.68 (s, 1H), 3.81 (d, 1H), 3.66 (d, 1H), 3.16-2.78 (m, 9H); Chiral analytical SFC: RT=5.23 min, Column: Chiralpak OD-3 (4.6×150 mm) 3 μm, 30% (0.5% DEA in Methanol), Flow rate: 3.0 g/min.

### 5-Cyano-N-(8,9-difluoro-6-oxo-1,2,3,4,5,6-hexahydrobenzo[c][1,7]naphthyridin-1-yl)-N-methyl-1H-indole-2-carboxamide (Compounds 405, 406)

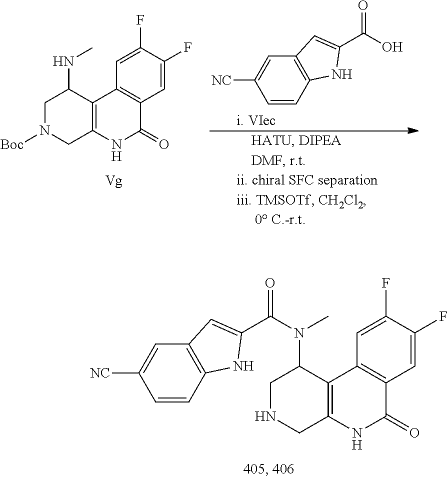 [SMILES: CNC1CN(C(=O)OC(C)(C)C)Cc2[nH]c(=O)c3cc(F)c(F)cc3c21 ; N#Cc1ccc2[nH]c(C(=O)O)cc2c1 ; CN(C(=O)c1cc2cc(C#N)ccc2[nH]1)C1CNCc2[nH]c(=O)c3cc(F)c(F)cc3c21]

Racemic tert-butyl 1-(5-cyano-N-methyl-1H-indole-2-carboxamido)-8,9-difluoro-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 5-cyano-1H-indole-2-carboxylic acid (VIec). The enantiomers were subsequently separated by preparative SFC: Method isocratic, Mobile phase (1:1 MeCN-methanol):CO₂—30:70. Column: Chiralpak IA-3 (30×250 mm) 5 μm, flow rate: 110 g/min, and each was converted to the final product in an analogous manner as described above (for Compounds 160, 161).

Enantiomer I (Compound 405): LCMS m/z found 434.2 [M+H]⁺; RT=2.96 min (Method A); ¹H NMR (400 MHz, DMSO-d₆) δ 12.29 (bs, 1H), 11.71 (bs, 1H), 8.18-8.10 (m, 2H), 7.63-7.54 (m, 2H), 7.27 (t, 1H), 7.06 (s, 1H), 5.76 (s, 1H), 3.90-3.77 (m, 2H), 3.17-3.12 (m, 5H), 2.93-2.91 (m, 1H); Chiral analytical SFC: RT=2.28 min, Column: Chiralpak AD-3 (4.6×150 mm) 3 μm, 50% (0.5% DEA in Methanol), Flow rate: 3.0 g/min.

Enantiomer II (Compound 406): LCMS m/z found 434.2 [M+H]⁺; RT=2.96 min (Method A); ¹H NMR (400 MHz, DMSO-d₆) δ 12.29 (bs, 1H), 11.71 (bs, 1H), 8.18-8.10 (m, 2H), 7.63-7.54 (m, 2H), 7.27 (t, 1H), 7.06 (s, 1H), 5.76 (s, 1H), 3.90-3.77 (m, 2H), 3.17-3.12 (m, 5H), 2.93-2.91 (m, 1H); Chiral analytical SFC: RT=3.26 min, Column: Chiralpak AD-3 (4.6×150 mm) 3 μm, 50% (0.5% DEA in Methanol), Flow rate: 3.0 g/min.

### 8-(Difluoromethyl)indolizine-2-carboxylic acid (VIes)

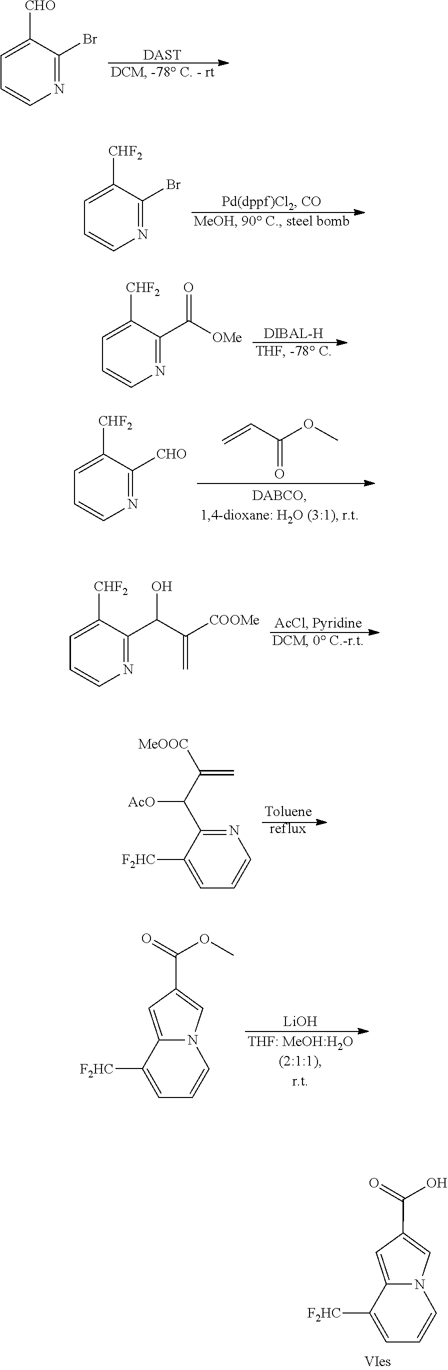

Step i. To a stirred solution of 2-bromonicotinaldehyde (10.0 g, 53.8 mmol, 1.0 eq.) in DCM (50 mL) was added dropwise DAST (21.3 mL, 161 mmol, 3 eq.) at −78° C. and the reaction mixture was stirred at room temperature for 5 h. The reaction mixture was poured slowly onto ice and extracted with DCM (2×200 mL). The organic layer was separated, washed with water, dried over Na₂SO₄ and concentrated under reduced pressure. The crude compound was purified by column chromatography using silica gel (100-200 mesh) and 10% EtOAc in petroleum ether as eluent to afford 2-bromo-3-(difluoromethyl)pyridine (5.5 g, 49% yield) of) as pale-yellow liquid. ¹H NMR (400 MHz, DMSO-d₆): 8.57 (d, 1H), 8.12 (d, 1H), 7.65 (m, 1H), 7.29 (t, 1H).

Step ii. To a stirred solution of 2-bromo-3-(difluoromethyl)pyridine (5.0 g, 24 mmol, 1.0 eq.) in MeOH (50 mL) was added Potassium Acetate (4.7 g, 48 mmol, 2 eq.) and Pd(dppf)Cl₂ (0.88 g, 1.2 mmol, 0.05 eq.) in a 250 mL steel reaction vessel under CO atmosphere (50 psi). The reaction mixture was heated to 90° C. and stirred for 16 h. The reaction mixture was filtered through a pad of CELITE® bed and the filtrate was evaporated. The residue was diluted with water (100 mL) and extracted with EtOAc (2×100 mL). Combined organic layer was washed with water, dried over Na₂SO₄ and concentrated under reduced pressure. The crude compound was purified by column chromatography using silica gel (100-200 mesh) and 15% EtOAc in petroleum ether as eluent to afford methyl 3-(difluoromethyl)picolinate (2.5 g, 55% yield) as a pale-yellow liquid. LCMS m/z found 188.1 [M+H]⁺; RT=1.30 min (Method E).

Step iii. To a stirred solution of methyl 3-(difluoromethyl)picolinate (2.50 g, 13.2 mmol, 1.0 eq.) in THF (28 mL) was added dropwise DIBAL-H (LOM in THF, 22.6 mL, 22.6 mmol, 1.7 eq.) at −78° C. and stirred for 3 h. The reaction mixture was quenched with aq. sat. NH₄Cl solution. Reaction mixture was filtered through a pad of CELITE®, the filtrate was dried over Na₂SO₄ and then concentrated under reduced pressure. The crude compound was purified by column chromatography using silica gel (100-200 mesh) and 10% EtOAc in petroleum ether as eluent to afford 3-(difluoromethyl)picolinaldehyde (1.4 g, 66% yield) as a pale-yellow liquid. LCMS m/z found 160.0 [M+H]⁺; RT=1.09 min (Method E).

Step iv. To a stirred solution of 3-(difluoromethyl)picolinaldehyde (1.60 g, 10.2 mmol, 1.0 eq.) in 1,4-dioxane:water (3:1, 50 mL) was added of methyl acrylate (1.10 mL, 12.2 mmol, 1.2 eq.) and DABCO (68 mg) at room temperature. The resulting reaction mixture was stirred at room temperature for 16 h. The reaction mixture was concentrated and diluted with water (40 mL) and extracted with EtOAc (2×70 mL). The organic layer was separated, dried over Na₂SO₄ and concentrated under reduced pressure. The crude compound was purified by column chromatography using silica gel (100-200 mesh) and 10% EtOAc in petroleum ether as eluent to afford methyl 2-((3-(difluoromethyl)pyridin-2-yl)(hydroxy)methyl)acrylate (1.5 g, 60% yield) as pale-yellow liquid. LCMS m/z found 244.0 [M+H]⁺; RT=1.16 min (Method E).

Step v. To a stirred solution of methyl 2-((3-(difluoromethyl)pyridin-2-yl)(hydroxy)methyl)acrylate (1.5 g, 6.2 mmol, 1.0 eq.) in DCM (15 mL) was added pyridine (0.84 mL, 9.3 mmol, 1.5 eq.) and acetyl chloride (0.69 mL, 8.6 mmol, 1.4 eq.) at 0° C. The resulting reaction mixture was stirred at room temperature for 3 h. The reaction mixture was diluted with water (40 mL) and extracted with EtOAc (2×100 mL). Combined organic layer was washed with water (50 mL), dried over Na₂SO₄ and concentrated under reduced pressure. The crude compound was diluted with toluene (15 mL) and refluxed for 16 h. The reaction mixture was concentrated under reduced pressure and purified by column chromatography using silica gel (100-200 mesh) and 10% EtOAc in petroleum ether as eluent to afford methyl 8-(difluoromethyl)indolizine-2-carboxylate (500 mg, 36% yield) as a pale-yellow liquid. LCMS m/z found 226.1 [M+H]⁺, RT=1.73 min (Method E).

Step vi. To a stirred solution of methyl 8-(difluoromethyl)indolizine-2-carboxylate (500 mg, 2.22 mmol, 1.0 eq.) in a mixture of THF:water:MeOH (2:1:1, 10 mL) was added LiOH (200 mg, 11.1 mmol, 5.0 eq.) at 0° C. and the reaction was stirred at room temperature for 4 h. The volatiles were evaporated from reaction mixture and acidified with aq. KHSO₄ solution (10 wt % in water) to pH˜2. The precipitated solids were collected by filtration, washed with water and dried under vacuum to afford 8-(difluoromethyl)indolizine-2-carboxylic acid (300 mg, 64% yield) as a white solid. LCMS m/z found 212.4 [M+H]⁺; RT=1.40 min (Method E).

### (S)-N-(8,9-difluoro-6-oxo-1,4,5,6-tetrahydro-2H-pyrano[3,4-c]isoquinolin-1-yl)-8-(difluoromethyl)-N-methylindolizine-2-carboxamide (Compound 407)

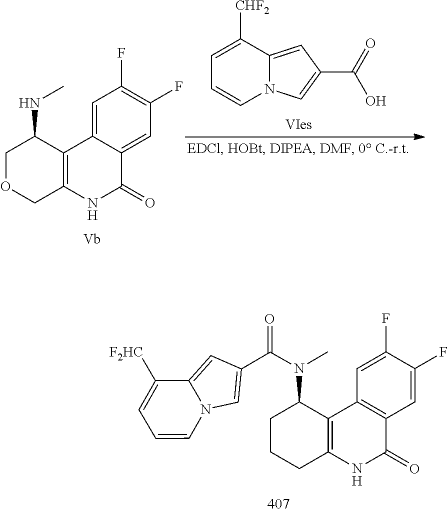

Enantiomerically pure (S)-N-(8,9-difluoro-6-oxo-1,4,5,6-tetrahydro-2H-pyrano[3,4-c]isoquinolin-1-yl)-8-(difluoromethyl)-N-methylindolizine-2-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 8-(difluoromethyl)indolizine-2-carboxylic acid (VIes). LCMS m/z found 460.2 [M+H]+; RT=3.80 min (Method A); ¹H NMR (400 MHz, DMSO-d₆) δ 11.69 (bs, 1H), 8.4 (d, 1H), 8.12 (t, 1H), 8.06 (s, 1H), 7.48-7.44 (m, 1H), 7.33-7.06 (m, 2H), 6.79 (s, 1H), 6.72 (t, 1H), 5.72 (s, 1H), 4.64 (d, 1H), 4.49 (d, 1H), 4.17 (d, 1H), 4.06 (d, 1H), 3.03 (s, 3H).

### (S)-N-(8,9-Difluoro-6-oxo-1,4,5,6-tetrahydro-2H-pyrano[3,4-c]isoquinolin-1-yl)-N-methyl-2,3-dihydro-1H-indene-5-carboxamide (Compound 408)

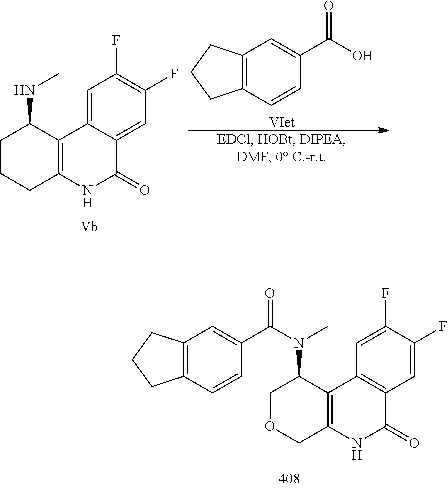

Enantiomerically pure (S)-N-(8,9-difluoro-6-oxo-1,4,5,6-tetrahydro-2H-pyrano[3,4-c]isoquinolin-1-yl)-N-methyl-2,3-dihydro-1H-indene-5-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 2,3-dihydro-1H-indene-5-carboxylic acid (VIet). LCMS m/z found 411.2 [M+H]⁺; RT=4.34 min (Method A); ¹H NMR (400 MHz, DMSO-d₆) δ 11.67 (bs, 1H), 8.13 (t, 1H), 7.52-7.47 (m, 1H), 7.29-7.24 (m, 2H), 7.12 (d, 1H), 5.68 (s, 1H), 4.58 (d, 1H), 4.46 (d, 1H), 4.23 (d, 1H), 4.03 (d, 1H), 2.87 (t, 4H), 2.70 (s, 3H), 2.02 (t, 2H).

### (S)-N-(8,9-Difluoro-6-oxo-1,4,5,6-tetrahydro-2H-pyrano[3,4-c]isoquinolin-1-yl)-N-methylbenzo[d]oxazole-6-carboxamide (Compound 409)

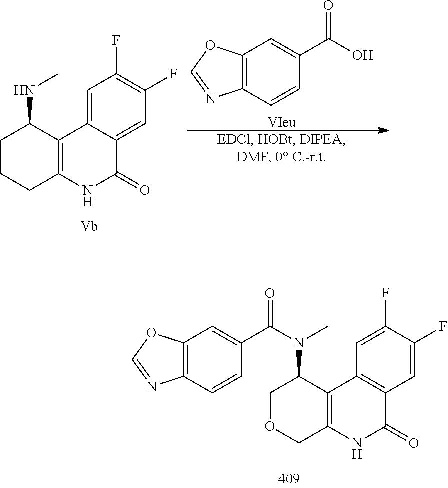

Enantiomerically pure (S)-N-(8,9-difluoro-6-oxo-1,4,5,6-tetrahydro-2H-pyrano[3,4-c]isoquinolin-1-yl)-N-methylbenzo[d]oxazole-6-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and benzo[d]oxazole-6-carboxylic acid (VIeu). LCMS m/z found 412.2 [M+H]⁺; RT=2.83 min (Method A); ¹H NMR (400 MHz, DMSO-d₆) δ 11.69 (bs, 1H), 8.85 (t, 1H), 8.14 (t, 1H), 7.88 (d, 1H), 7.56-7.51 (m, 1H), 7.41 (d, 1H), 5.761 (s, 1H), 4.59 (d, 1H), 4.47 (d, 1H), 4.31 (d, 1H), 4.07-4.03 (m, 1H), 2.73 (s, 3H).

### (S)-N-(8,9-Difluoro-6-oxo-1,4,5,6-tetrahydro-2H-pyrano[3,4-c]isoquinolin-1-yl)-5-fluoro-N-methyl-6-(trifluoromethyl)nicotinamide (Compound 412)

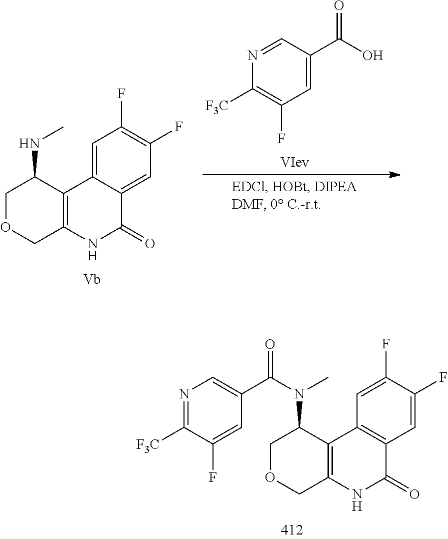 [SMILES: CN[C@@H]1COCc2[nH]c(=O)c3cc(F)c(F)cc3c21 ; O=C(O)c1cnc(C(F)(F)F)c(F)c1 ; CN(C(=O)c1cnc(C(F)(F)F)c(F)c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-5-fluoro-N-methyl-6-(trifluoromethyl)nicotinamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 5-fluoro-6-(trifluoromethyl)nicotinic acid (VIev). LCMS m/z found 458.2 [M+H]⁺; RT=4.15 min (Method A); ¹H NMR (400 MHz, DMSO-d₆) δ 11.70 (bs, 1H), 8.67 (bs, 1H), 8.31 (d, 1H), 8.16 (t, 1H), 7.50 (m, 1H), 5.66 (s, 1H), 4.63 (d, 1H), 4.49 (d, 1H), 4.35 (d, 1H), 4.03 (d, 1H), 2.93 (s, 3H).

### 5-Fluoro-N-(8-fluoro-6-oxo-1,4,5,6-tetrahydro-2H-pyrano[3,4-c]isoquinolin-1-yl)-N-methyl-6-(trifluoromethyl)nicotinamide (Compound 413, 414)

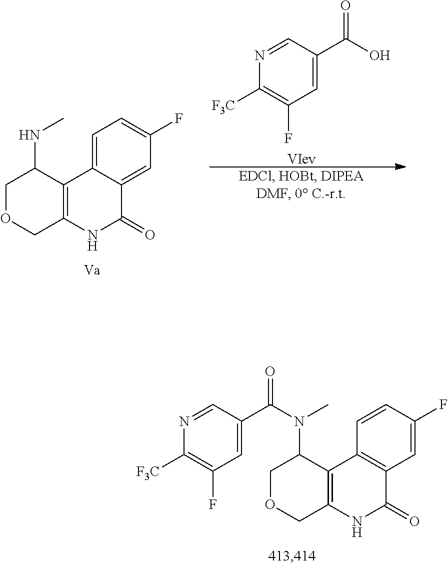 [SMILES: CNC1COCc2[nH]c(=O)c3cc(F)ccc3c21 ; O=C(O)c1cnc(C(F)(F)F)c(F)c1 ; CN(C(=O)c1cnc(C(F)(F)F)c(F)c1)C1COCc2[nH]c(=O)c3cc(F)ccc3c21]

Racemic 5-fluoro-N-(8-fluoro-6-oxo-1,4,5,6-tetrahydro-2H-pyrano[3,4-c]isoquinolin-1-yl)-N-methyl-6-(trifluoromethyl)nicotinamide was synthesized in an analogous manner as described above from 8-fluoro-1-(methylamino)-1,5-dihydro-2H-pyrano[3,4-c]isoquinolin-6(4H)-one (Va) and 5-fluoro-6-(trifluoromethyl)nicotinic acid (VIev). The enantiomers were subsequently separated by preparative SFC: Method isocratic, Mobile phase methanol:CO₂—30:70. Column: Chiralpak IG (30×250 mm), 5 μm, flow rate: 110 g/min.

Enantiomer I (Compound 413): LCMS m/z found 440.2 [M+H]⁺; RT=3.89 min (Method A); ¹H NMR (400 MHz, DMSO-d₆) δ 11.61 (bs, 1H), 8.69 (bs, 1H), 8.30 (bs, 1H), 7.91 (d, 1H), 7.77 (t, 1H), 7.63 (m, 1H), 5.69 (s, 1H), 4.62 (d, 1H), 4.49 (d, 1H), 4.33 (d, 1H), 4.05 (d, 1H), 2.72 (s, 3H); Chiral analytical SFC: RT=1.03 min, Column: Chiralpak IG-3 (4.6×150 mm) 3 μm, 40% (0.5% DEA in Methanol), Flow rate: 3.0 g/min.

Enantiomer II (Compound 414): LCMS m/z found 440.2 [M+H]⁺; RT=3.89 min (Method A); ¹H NMR (400 MHz, DMSO-d₆) δ 11.61 (bs, 1H), 8.69 (bs, 1H), 8.30 (bs, 1H), 7.91 (d, 1H), 7.77 (t, 1H), 7.63 (m, 1H), 5.69 (s, 1H), 4.62 (d, 1H), 4.49 (d, 1H), 4.33 (d, 1H), 4.05 (d, 1H), 2.72 (s, 3H); Chiral analytical SFC: RT=2.42 min, Column: Chiralpak IG-3 (4.6×150 mm) 3 μm, 40% (0.5% DEA in Methanol), Flow rate: 3.0 g/min.

### 5-Chloro-N-(8,9-difluoro-6-oxo-1,2,3,4,5,6-hexahydrobenzo[c][1,7]naphthyridin-1-yl)-N-methyl-6-(trifluoromethyl)nicotinamide (Compounds 418, 419)

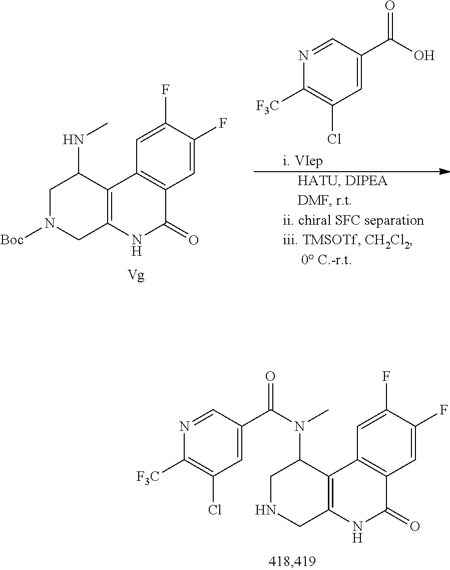 [SMILES: CNC1CN(C(=O)OC(C)(C)C)Cc2[nH]c(=O)c3cc(F)c(F)cc3c21 ; O=C(O)c1cnc(C(F)(F)F)c(Cl)c1 ; CN(C(=O)c1cnc(C(F)(F)F)c(Cl)c1)C1CNCc2[nH]c(=O)c3cc(F)c(F)cc3c21]

Racemic tert-butyl 1-(5-chloro-N-methyl-6-(trifluoromethyl)nicotinamido)-8,9-difluoro-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 5-chloro-6-(trifluoromethyl)nicotinic acid (VIep). The enantiomers were subsequently separated by preparative SFC: Method isocratic, Mobile phase Methanol:CO₂—40:60. Column: Chiralpak IC (30×250 mm) 5 μm, flow rate: 110 g/min, and each was converted to the final product in an analogous manner as described above (for Compounds 160, 161).

Enantiomer I (Compound 418): LCMS m/z found 473.1 [M+H]⁺; RT=3.42 min (Method A); ¹H NMR (400 MHz, DMSO-d₆) δ 11.62 (bs, 1H), 8.73 (bs, 1H), 8.41 (bs, 1H), 8.13 (t, 1H), 7.38 (m, 1H), 5.55 (s, 1H), 3.80 (d, 1H), 3.67 (d, 1H), 3.36 (d, 1H), 3.12 (d, 1H), 2.76 (s, 4H); Chiral analytical SFC: RT=0.79 min, Column: Chiralpak AD-3 (4.6×150 mm) 3 μm, 40% (0.5% DEA in Methanol), Flow rate: 3.0 g/min.

Enantiomer II (Compound 419): LCMS m/z found 473.1 [M+H]⁺; RT=3.42 min (Method A); ¹H NMR (400 MHz, DMSO-d₆) δ 11.62 (bs, 1H), 8.73 (bs, 1H), 8.41 (bs, 1H), 8.13 (t, 1H), 7.38 (m, 1H), 5.55 (s, 1H), 3.80 (d, 1H), 3.67 (d, 1H), 3.36 (d, 1H), 3.12 (d, 1H), 2.76 (s, 4H); Chiral analytical SFC: RT=1.61 min, Column: Chiralpak AD-3 (4.6×150 mm) 3 μm, 40% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-N-methyl-4H-thieno[3,2-b]pyrrole-5-carboxamide (Compounds 420, 421)

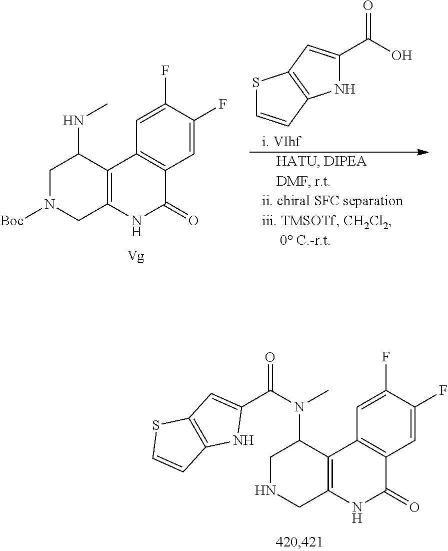

Racemic tert-butyl 8,9-difluoro-1-(N-methyl-4H-thieno[3,2-b]pyrrole-5-carboxamido)-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 4H-thieno[3,2-b]pyrrole-5-carboxylic acid (VIhf). The enantiomers were subsequently separated by preparative SFC: Method isocratic, Mobile phase Methanol:CO₂—40:60. Column: Chiralpak IC-3 (30×250 mm), 5μ, flow rate: 110 g/min, and each was converted to the final product in an analogous manner as described above (for Compounds 160, 161).

Enantiomer I (Compound 420): LCMS m/z found 415.1 [M+H]⁺; RT=3.03 min (Method A); ¹H NMR (400 MHz, DMSO-d₆) δ 11.80 (bs, 1H), 11.63 (bs, 1H), 8.12 (t, 1H), 7.43 (d, 1H), 7.28 (m, 1H), 7.00 (d, 1H), 6.92 (bs, 1H), 5.61 (s, 1H), 3.82 (d, 1H), 3.66 (d, 1H), 3.11-2.65 (m, 6H); Chiral analytical SFC: RT=5.70 min, Column: Chiralpak OD-3 (4.6×150 mm) 3 μm, 40% (0.5% DEA in Methanol), Flow rate: 3.0 g/min.

Enantiomer II (Compound 421): LCMS m/z found 415.1 [M+H]⁺; RT=3.03 min (Method A); ¹H NMR (400 MHz, DMSO-d₆) δ 11.80 (bs, 1H), 11.63 (bs, 1H), 8.12 (t, 1H), 7.43 (d, 1H), 7.28 (m, 1H), 7.00 (d, 1H), 6.92 (bs, 1H), 5.61 (s, 1H), 3.82 (d, 1H), 3.66 (d, 1H), 3.11-2.65 (m, 6H); Chiral analytical SFC: RT=7.70 min, Column: Chiralpak OD-3 (4.6×150 mm) 3 μm, 40% (0.5% DEA in Methanol), Flow rate: 3.0 g/min.

### N-(8-Fluoro-6-oxo-1,4,5,6-tetrahydro-2H-pyrano[3,4-c]isoquinolin-1-yl)-N,4-dimethyl-1H-indole-2-carboxamide (Compounds 422, 423)

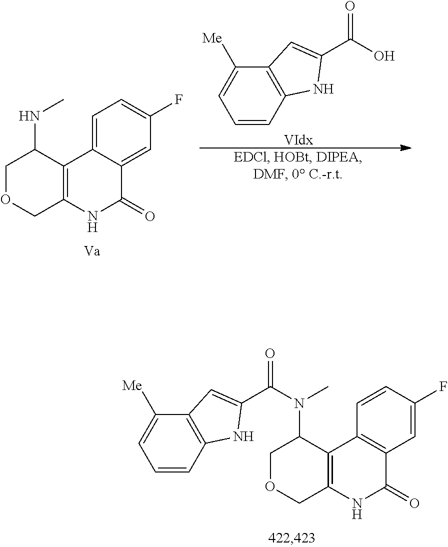

Racemic N-(8-fluoro-6-oxo-1,4,5,6-tetrahydro-2H-pyrano[3,4-c]isoquinolin-1-yl)-N,4-dimethyl-1H-indole-2-carboxamide was synthesized in an analogous manner as described above from 8-fluoro-1-(methylamino)-1,5-dihydro-2H-pyrano[3,4-c]isoquinolin-6(4H)-one (Va) and 4-methyl-1H-indole-2-carboxylic acid (VIdx). The enantiomers were subsequently separated by preparative SFC: Method isocratic, Mobile phase methanol:CO₂—40:60. Column: Chiralpak IA-3 (30×250 mm) 5 μm, flow rate: 110 g/min.

Enantiomer I (Compound 422): LCMS m/z found 406.2 [M+H]⁺; RT=4.15 min (Method A); ¹H NMR (400 MHz, DMSO-d₆) δ 11.64 (bs, 2H), 7.91 (q, 1H), 7.64 (t, 1H), 7.5 (bs, 1H), 7.2 (d, 1H), 7.1 (t, 1H),) 6.9 (d, 1H), 6.67 (d, 1H), 5.77 (s, 1H), 4.59 (d, 1H), 4.47 (d, 1H), 4.22 (d, 1H), 4.04 (d, 1H), 3.1 (s, 3H), 2.4 (s, 3H); Chiral analytical SFC: RT=2.49 min, Column: Chiralpak IA-3 (4.6×150 mm) 3 μm, 40% (Acetonitrile:Methanol)(1:1), Flow rate: 3.0 g/min.

Enantiomer II (Compound 423): LCMS m/z found 406.2 [M+H]⁺; RT=4.15 min (Method A); ¹H NMR (400 MHz, DMSO-d₆) δ 11.64 (bs, 2H), 7.91 (q, 1H), 7.64 (t, 1H), 7.5 (bs, 1H), 7.2 (d, 1H), 7.1 (t, 1H),) 6.9 (d, 1H), 6.67 (d, 1H), 5.77 (s, 1H), 4.59 (d, 1H), 4.47 (d, 1H), 4.22 (d, 1H), 4.04 (d, 1H), 3.1 (s, 3H), 2.4 (s, 3H); Chiral analytical SFC: RT=9.61 min, Column: Chiralpak IA-3 (4.6×150 mm) 3 μm, 40% (Acetonitrile:Methanol)(1:1), Flow rate: 3.0 g/min.

### 4-Chloro-N-(8-fluoro-6-oxo-1,4,5,6-tetrahydro-2H-pyrano[3,4-c]isoquinolin-1-yl)-N-methyl-1H-indole-2-carboxamide (Compounds 424, 425)

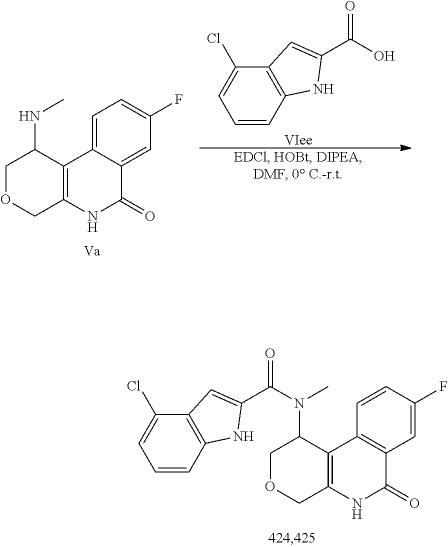

Racemic 4-chloro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from 8-fluoro-1-(methylamino)-1,5-dihydro-2H-pyrano[3,4-c]isoquinolin-6(4H)-one (Va) and 4-chloro-1H-indole-2-carboxylic acid (VIee). The enantiomers were subsequently separated by preparative SFC: Method isocratic, Mobile phase (Acetonitrile:Methanol)(1:1):CO₂—50:50. Column: Chiralpak IA-3 (30×250 mm), 5 μm, flow rate: 110 g/min.

Enantiomer I (Compound 424): LCMS m/z found 426.1 [M+H]⁺; RT=4.35 min (Method A); ¹H NMR (400 MHz, DMSO-d₆) δ 12.08 (bs, 1H), 11.64 (bs, 1H), 7.92 (d, 1H), 7.62 (t, 1H), 7.55 (bs, 1H), 7.46 (d, 1H), 7.23 (t, 1H), 7.14 (d, 1H), 6.85 (bs, 1H), 5.76 (s, 1H), 4.66 (d, 1H), 4.51 (d, 1H), 4.20 (d, 1H), 4.07 (d, 1H), 3.16 (s, 3H); Chiral analytical SFC: RT=2.49 min, Column: Chiralpak IA-3 (4.6×150 mm) 3 μm, 40% (Acetonitrile:Methanol)(1:1), Flow rate: 3.0 g/min.

Enantiomer II (Compound 425): LCMS m/z found 406.2 [M+H]⁺; RT=4.15 min (Method A); ¹H NMR (400 MHz, DMSO-d₆) δ 12.08 (bs, 1H), 11.64 (bs, 1H), 7.92 (d, 1H), 7.62 (t, 1H), 7.55 (bs, 1H), 7.46 (d, 1H), 7.23 (t, 1H), 7.14 (d, 1H), 6.85 (bs, 1H), 5.76 (s, 1H), 4.66 (d, 1H), 4.51 (d, 1H), 4.20 (d, 1H), 4.07 (d, 1H), 3.16 (s, 3H); Chiral analytical SFC: RT=8.99 min, Column: Chiralpak IA-3 (4.6×150 mm) 3 μm, 40% (Acetonitrile:Methanol)(1:1), Flow rate: 3.0 g/min.

### (S)-N-(8,9-Difluoro-6-oxo-1,4,5,6-tetrahydro-2H-pyrano[3,4-c]isoquinolin-1-yl)-N-methylbenzo[d]oxazole-5-carboxamide (Compound 426)

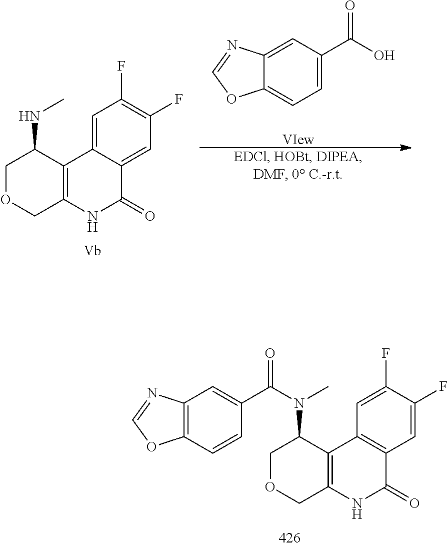

Enantiomerically pure (S)-N-(8,9-difluoro-6-oxo-1,4,5,6-tetrahydro-2H-pyrano[3,4-c]isoquinolin-1-yl)-N-methylbenzo[d]oxazole-5-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and benzo[d]oxazole-5-carboxylic acid (VIew). LCMS m/z found 412.1 [M+H]⁺; RT=2.89 min (Method A); ¹H NMR (400 MHz, DMSO-d₆) δ 11.69 (bs, 1H), 8.84 (bs, 1H), 8.16 (t, 1H), 7.87 (d, 2H), 7.57-7.45 (m, 2H), 5.71 (s, 1H), 4.61 (d, 1H), 4.49 (d, 1H), 4.33 (d, 1H), 4.06 (m, 1H), 2.73 (s, 3H).

### (S)-N-(8,9-Difluoro-6-oxo-1,4,5,6-tetrahydro-2H-pyrano[3,4-c]isoquinolin-1-yl)-N-methylbenzo[d]thiazole-5-carboxamide (Compound 427)

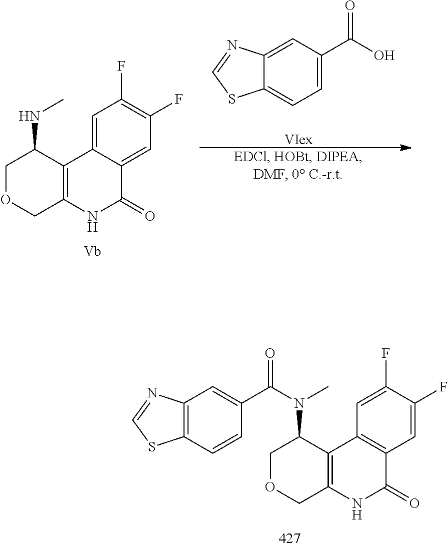

Enantiomerically pure (S)-N-(8,9-difluoro-6-oxo-1,4,5,6-tetrahydro-2H-pyrano[3,4-c]isoquinolin-1-yl)-N-methylbenzo[d]thiazole-5-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and benzo[d]thiazole-5-carboxylic acid (VIex). LCMS m/z found 428.1 [M+H]⁺; RT=3.14 min (Method A); ¹H NMR (400 MHz, DMSO-d₆) δ 11.69 (bs, 1H), 9.48 (t, 1H), 8.28 (d, 1H), 8.17 (t, 2H), 7.59-7.49 (m, 2H), 5.74 (s, 1H), 4.62 (d, 1H), 4.50 (d, 1H), 4.35 (d, 1H), 4.08 (m, 1H), 2.75 (s, 3H).

### (S)-N-(8,9-Difluoro-6-oxo-1,4,5,6-tetrahydro-2H-pyrano[3,4-c]isoquinolin-1-yl)-N-methylbenzo[d]thiazole-6-carboxamide (Compound 428)

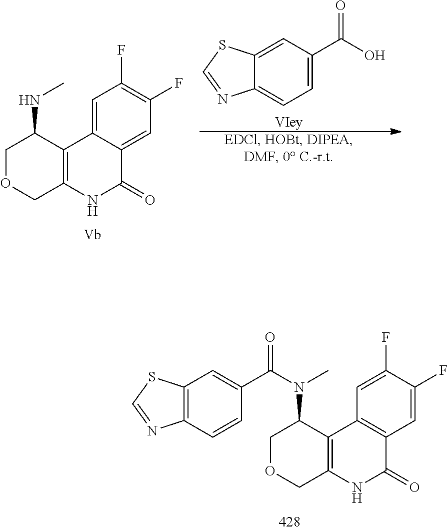

Enantiomerically pure (S)-N-(8,9-difluoro-6-oxo-1,4,5,6-tetrahydro-2H-pyrano[3,4-c]isoquinolin-1-yl)-N-methylbenzo[d]thiazole-6-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and benzo[d]thiazole-6-carboxylic acid (VIey). LCMS m/z found 428.1 [M+H]⁺; RT=3.06 min (Method A); ¹H NMR (400 MHz, DMSO-d₆) δ 11.69 (bs, 1H), 9.48 (s, 1H), 8.30 (t, 1H), 8.16 (t, 1H), 7.56-7.52 (m, 2H), 5.72 (s, 1H), 4.62 (d, 1H), 4.50 (d, 1H), 4.31 (d, 1H), 4.07 (d, 1H), 2.75 (s, 3H).

### (S)-N-(8,9-Difluoro-6-oxo-1,4,5,6-tetrahydro-2H-pyrano[3,4-c]isoquinolin-1-yl)-N-methyl-1H-indazole-5-carboxamide (Compound 429)

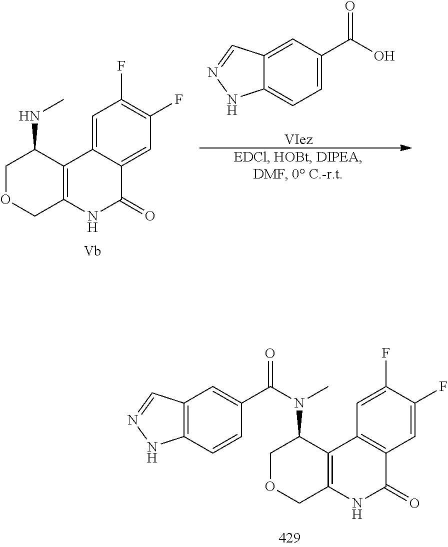

Enantiomerically pure (S)-N-(8,9-difluoro-6-oxo-1,4,5,6-tetrahydro-2H-pyrano[3,4-c]isoquinolin-1-yl)-N-methyl-1H-indazole-5-carboxamide was synthesized in an analogous manner as described above from enantiomerically pure (1S)-8,9-difluoro-1-(methylamino)-1,2,4,5-tetrahydropyrano[3,4-c]isoquinolin-6-one (Vb) and 1H-indazole-5-carboxylic acid (VIez). LCMS m/z found 411.3 [M+H]⁺; RT=1.42 min (Method E); ¹H NMR (400 MHz, DMSO-d₆) δ 13.25 (bs, 1H), 11.66 (bs, 1H), 8.15 (t, 1H), 7.86 (d, 1H), 7.61 (m, 1H), 7.39 (md 1H), 5.71 (s, 1H), 4.61 (d, 1H), 4.49 (d, 1H), 4.29 (d, 1H), 4.07 (d, 1H), 2.77 (s, 3H).

### N-(8,9-Difluoro-6-oxo-1,2,3,4,5,6-hexahydrobenzo[c][1,7]naphthyridin-1-yl)-8-(difluoromethyl)-N-methylindolizine-2-carboxamide (Compounds 430, 431)

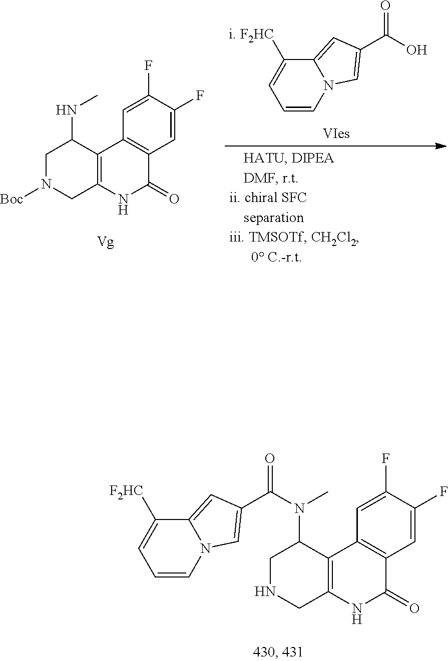 [SMILES: CNC1CN(C(=O)OC(C)(C)C)Cc2[nH]c(=O)c3cc(F)c(F)cc3c21 ; O=C(O)c1cc2c(C(F)F)cccn2c1 ; CN(C(=O)c1cc2c(C(F)F)cccn2c1)C1CNCc2[nH]c(=O)c3cc(F)c(F)cc3c21]

Racemic tert-butyl 1-(8-(difluoromethyl)-N-methylindolizine-2-carboxamido)-8,9-difluoro-6-oxo-1,4,5,6-tetrahydrobenzo[c][1,7]naphthyridine-3(2H)-carboxylate was synthesized in an analogous manner as described above from racemic tert-butyl 8,9-difluoro-1-(methylamino)-6-oxo-1,4,5,6-tetrahydrobenzo[c][1,7]naphthyridine-3(2H)-carboxylate (Vg) and 8-(difluoromethyl)indolizine-2-carboxylic acid (VIes). The enantiomers were subsequently separated by preparative SFC: Method isocratic, Mobile phase Methanol:CO₂—40:60. Column: Lux Cellulose (30×250 mm), 5 μm, flow rate: 110 g/min, and each was converted to the final product in an analogous manner as described above (for Compounds 160, 161).

Enantiomer I (Compound 430): LCMS m/z found 459.1 [M+H]⁺; RT=3.01 min (Method A); ¹H NMR (400 MHz, DMSO-d₆) δ 11.61 (bs, 1H), 8.40 (d, 1H), 8.13 (m, 2H), 7.32-7.05 (m, 3H), 6.78-6.95 (m, 2H), 5.65 (s, 1H), 3.80 (d, 1H), 3.66 (d, 1H), 3.50 (d, 1H), 3.48 (d, 1H), 2.76 (s, 4H); Chiral analytical SFC: RT=4.63 min, Column: Chiralpak IG-3 (4.6×150 mm) 3 μm, 40% (0.5% DEA in Methanol), Flow rate: 3.0 g/min.

Enantiomer II (Compound 431): LCMS m/z found 459.1 [M+H]⁺; RT=3.01 min (Method A); ¹H NMR (400 MHz, DMSO-d₆) δ 11.61 (bs, 1H), 8.40 (d, 1H), 8.13 (m, 2H), 7.32-7.05 (m, 3H), 6.78-6.95 (m, 2H), 5.65 (s, 1H), 3.80 (d, 1H), 3.66 (d, 1H), 3.50 (d, 1H), 3.48 (d, 1H), 2.76 (s, 4H); Chiral analytical SFC: RT=6.33 min, Column: Chiralpak IG-3 (4.6×150 mm) 3 μm, 40% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-5-(difluoromethyl)-N-methylindolizine-2-carboxamide (Compounds 432, 433)

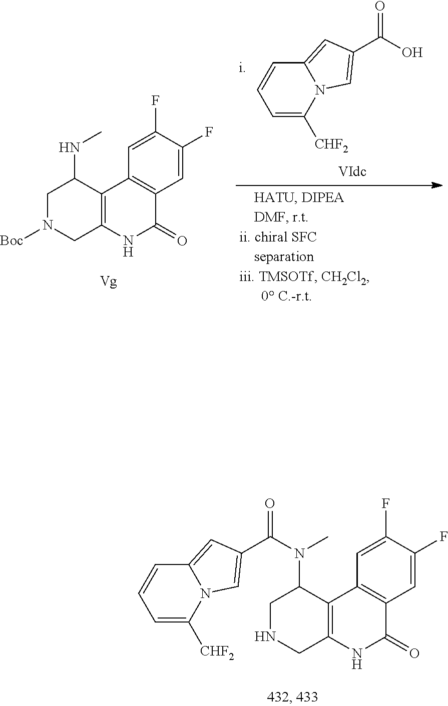 [SMILES: CNC1CN(C(=O)OC(C)(C)C)Cc2[nH]c(=O)c3cc(F)c(F)cc3c21 ; O=C(O)c1cc2cccc(C(F)F)n2c1 ; CN(C(=O)c1cc2cccc(C(F)F)n2c1)C1CNCc2[nH]c(=O)c3cc(F)c(F)cc3c21]

Racemic tert-butyl 1-(5-(difluoromethyl)-N-methylindolizine-2-carboxamido)-8,9-difluoro-6-oxo-1,4,5,6-tetrahydrobenzo[c][1,7]naphthyridine-3(2H)-carboxylatewas synthesized in an analogous manner as described above from racemic tert-butyl 8,9-difluoro-1-(methylamino)-6-oxo-1,4,5,6-tetrahydrobenzo[c][1,7]naphthyridine-3(2H)-carboxylate (Vg) and 5-(difluoromethyl)indolizine-2-carboxylic acid (VIdc). The enantiomers were subsequently separated by preparative SFC: Method isocratic, Mobile phase [(0.2% 7 M methanolic ammonia (Methanol:ACN)(1:1)]:CO₂—40:60. Column: Lux Cellulose-2 (30×250 mm), 5 μm, flow rate: 110 g/min, and each was converted to the final product in an analogous manner as described above (for Compounds 160, 161).

Enantiomer I (Compound 432): LCMS m/z found 459.1 [M+H]⁺; RT=3.09 min (Method A); ¹H NMR (400 MHz, DMSO-d₆) δ 11.61 (bs, 1H), 8.13 (bs, 1H), 7.90 (bs, 1H), 7.75 (d, 1H), 7.54-7.28 (m, 2H), 7.07 (d, 1H), 6.93 (m, 2H), 5.64 (s, 1H), 3.87 (d, 1H), 3.80 (d, 1H), 3.2-3.13 (m, 2H), 2.99 (s, 4H); Chiral analytical SFC: RT=3.60 min, Column: Chiralpak IG-3 (4.6×150 mm) 3 μm, 40% (0.5% DEA in Methanol), Flow rate: 3.0 g/min.

Enantiomer II (Compound 433): LCMS m/z found 459.1 [M+H]⁺; RT=3.09 min (Method A); ¹H NMR (400 MHz, DMSO-d₆) δ 11.61 (bs, 1H), 8.13 (bs, 1H), 7.90 (bs, 1H), 7.75 (d, 1H), 7.54-7.28 (m, 2H), 7.07 (d, 1H), 6.93 (m, 2H), 5.64 (s, 1H), 3.87 (d, 1H), 3.80 (d, 1H), 3.2-3.13 (m, 2H), 2.99 (s, 4H); Chiral analytical SFC: RT=6.08 min, Column: Chiralpak IG-3 (4.6×150 mm) 3 μm, 40% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-5-fluoro-N-methyl-6-(trifluoromethyl)nicotinamide (Compounds 434, 435)

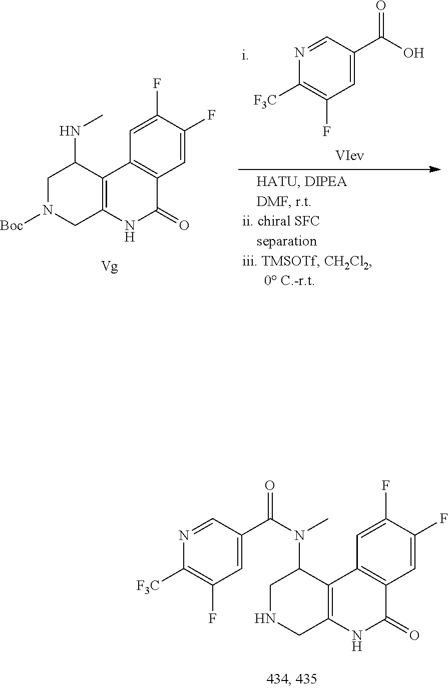 [SMILES: CNC1CN(C(=O)OC(C)(C)C)Cc2[nH]c(=O)c3cc(F)c(F)cc3c21 ; O=C(O)c1cnc(C(F)(F)F)c(F)c1 ; CN(C(=O)c1cnc(C(F)(F)F)c(F)c1)C1CNCc2[nH]c(=O)c3cc(F)c(F)cc3c21]

Racemic tert-butyl 8,9-difluoro-1-(5-fluoro-N-methyl-6-(trifluoromethyl)nicotinamido)-6-oxo-1,4,5,6-tetrahydrobenzo[c][1,7]naphthyridine-3(2H)-carboxylate was synthesized in an analogous manner as described above from racemic tert-butyl 8,9-difluoro-1-(methylamino)-6-oxo-1,4,5,6-tetrahydrobenzo[c][1,7]naphthyridine-3(2H)-carboxylate (Vg) and 5-fluoro-6-(trifluoromethyl)nicotinic acid (VIev). The enantiomers were subsequently separated by preparative SFC: Method isocratic, Mobile phase Methanol:CO₂—40:60. Column: Chiralpak IC (30×250 mm), 5 μm, flow rate: 110 g/min, and each was converted to the final product in an analogous manner as described above (for Compounds 160, 161).

Enantiomer I (Compound 434): LCMS m/z found 457.1 [M+H]⁺; RT=3.19 min (Method A); ¹H NMR (400 MHz, DMSO-d₆) δ 11.63 (bs, 1H), 8.64 (bs, 1H), 8.29 (d, 1H), 8.14 (t, 1H), 7.36 (m, 1H), 5.55 (s, 1H), 3.80 (d, 1H), 3.67 (d, 1H), 3.32 (d, 1H), 3.14 (d, 1H), 2.76 (s, 4H); Chiral analytical SFC: RT=0.90 min, Column: Chiralpak AD-3 (4.6×150 mm) 3 μm, 40% (0.5% DEA in Methanol), Flow rate: 3.0 g/min.

Enantiomer II (Compound 435): LCMS m/z found 457.1 [M+H]⁺; RT=3.19 min (Method A); ¹H NMR (400 MHz, DMSO-d₆) δ 11.63 (bs, 1H), 8.64 (bs, 1H), 8.29 (d, 1H), 8.14 (t, 1H), 7.36 (m, 1H), 5.55 (s, 1H), 3.80 (d, 1H), 3.67 (d, 1H), 3.32 (d, 1H), 3.14 (d, 1H), 2.76 (s, 4H); Chiral analytical SFC: RT=1.84 min, Column: Chiralpak AD-3 (4.6×150 mm) 3 μm, 40% (0.5% DEA in Methanol), Flow rate: 3.0 g/min.

### 4-Chloro-6-fluoro-N-(8-fluoro-6-oxo-1,4,5,6-tetrahydro-2H-pyrano[3,4-c]isoquinolin-1-yl)-N-methyl-1H-indole-2-carboxamide (Compounds 438, 439)

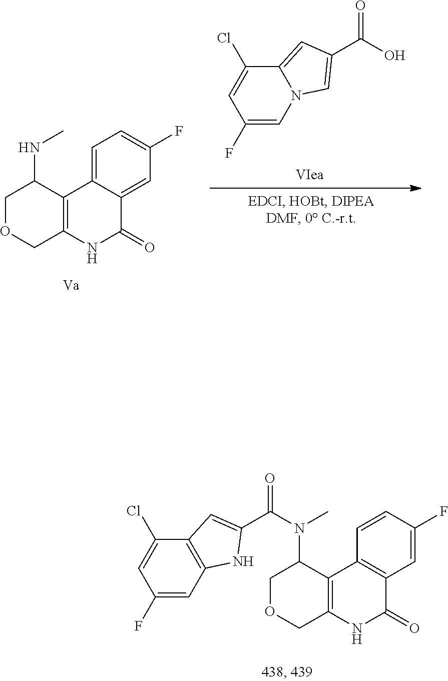 [SMILES: CNC1COCc2[nH]c(=O)c3cc(F)ccc3c21 ; O=C(O)c1cc2c(Cl)cc(F)cn2c1 ; CN(C(=O)c1cc2c(Cl)cc(F)cc2[nH]1)C1COCc2[nH]c(=O)c3cc(F)ccc3c21]

Racemic 4-chloro-6-fluoro-N-(8-fluoro-6-oxo-1,4,5,6-tetrahydro-2H-pyrano[3,4-c]isoquinolin-1-yl)-N-methyl-1H-indole-2-carboxamide was synthesized in an analogous manner as described above from 8-fluoro-1-(methylamino)-1,5-dihydro-2H-pyrano[3,4-c]isoquinolin-6(4H)-one (Va) and 4-chloro-6-fluoro-1H-indole-2-carboxylic acid (VIea). The enantiomers were subsequently separated by preparative SFC: Method isocratic, Mobile phase (Acetonitrile:Methanol)(1:1):CO₂—50:50. Column: Chiralpak IA-3 (30×250 mm), 5μ, flow rate: 110 g/min.

Enantiomer I (Compound 438): LCMS m/z found 444.0 [M+H]⁺; RT=4.59 min (Method A); ¹H NMR (400 MHz, DMSO-d₆) δ 11.90 (bs, 2H), 7.90 (d, 1H), 7.62-7.50 (m, 2H), 7.21-7.13 (m, 2H), 6.87 (bs, 1H), 5.74 (s, 1H), 4.64 (d, 1H), 4.49 (d, 1H), 4.19 (d, 1H), 4.06 (d, 1H), 3.15 (s, 3H); Chiral analytical SFC: RT=2.20 min, Column: Chiralpak IA-3 (4.6×150 mm) 3 μm, 40% (Acetonitrile:Methanol)(1:1), Flow rate: 3.0 g/min.

Enantiomer II (Compound 439): LCMS m/z found 444.0 [M+H]⁺; RT=4.59 min (Method A); ¹H NMR (400 MHz, DMSO-d₆) δ 11.90 (bs, 2H), 7.90 (d, 1H), 7.62-7.50 (m, 2H), 7.21-7.13 (m, 2H), 6.87 (bs, 1H), 5.74 (s, 1H), 4.64 (d, 1H), 4.49 (d, 1H), 4.19 (d, 1H), 4.06 (d, 1H), 3.15 (s, 3H); Chiral analytical SFC: RT=6.17 min, Column: Chiralpak IA-3 (4.6×150 mm) 3 μm, 40% (Acetonitrile:Methanol)(1:1), Flow rate: 3.0 g/min.

### 4-Chloro-N-(8,9-difluoro-6-oxo-1,2,3,4,5,6-hexahydrobenzo[c][1,7]naphthyridin-1-yl)-N-methyl-1H-indole-2-carboxamide (Compounds 440, 441)

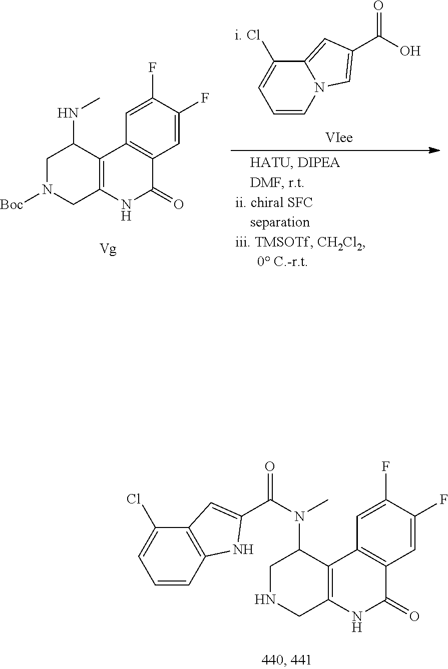 [SMILES: CNC1CN(C(=O)OC(C)(C)C)Cc2[nH]c(=O)c3cc(F)c(F)cc3c21 ; O=C(O)c1cc2c(Cl)cccn2c1 ; CN(C(=O)c1cc2c(Cl)cccc2[nH]1)C1CNCc2[nH]c(=O)c3cc(F)c(F)cc3c21]

Racemic tert-butyl 1-(4-chloro-N-methyl-1H-indole-2-carboxamido)-8,9-difluoro-6-oxo-1,4,5,6-tetrahydrobenzo[c][1,7]naphthyridine-3(2H)-carboxylate was synthesized in an analogous manner as described above from racemic tert-butyl 8,9-difluoro-1-(methylamino)-6-oxo-1,4,5,6-tetrahydrobenzo[c][1,7]naphthyridine-3(2H)-carboxylate (Vg) and 4-chloro-1H-indole-2-carboxylic acid (VIee). The enantiomers were subsequently separated by preparative SFC: Method isocratic, Mobile phase Methanol:CO₂—30:70. Column: Chiralpak IA (30×250 mm), 5 μm, flow rate: 110 g/min, and each was converted to the final product in an analogous manner as described above (for Compounds 160, 161).

Enantiomer I (Compound 440): LCMS m/z found 443.1 [M+H]⁺; RT=3.63 min (Method A); ¹H NMR (400 MHz, DMSO-d₆) δ 12.11 (bs, 1H), 11.74 (bs, 1H), 8.14 (t, 1H), 7.45 (d, 1H), 7.33-7.28 (m, 1H), 7.22 (t, 1H), 7.14 (d, 1H), 6.89 (s, 1H), 5.81 (s, 1H), 4.10-3.80 (m, 2H), 3.30-3.20 (m, 2H), 3.12 (s, 3H); Chiral analytical SFC: RT=2.79 min, Column: Chiralpak AD-3 (4.6×150 mm) 3 μm, 40% (0.5% DEA in Methanol), Flow rate: 3.0 g/min.

Enantiomer II (Compound 441): LCMS m/z found 443.1 [M+H]⁺; RT=3.63 min (Method A); ¹H NMR (400 MHz, DMSO-d₆) δ 12.11 (bs, 1H), 11.74 (bs, 1H), 8.14 (t, 1H), 7.45 (d, 1H), 7.33-7.28 (m, 1H), 7.22 (t, 1H), 7.14 (d, 1H), 6.89 (s, 1H), 5.81 (s, 1H), 4.10-3.80 (m, 2H), 3.30-3.20 (m, 2H), 3.12 (s, 3H); Chiral analytical SFC: RT=8.01 min, Column: Chiralpak AD-3 (4.6×150 mm) 3 μm, 40% (0.5% DEA in Methanol), Flow rate: 3.0 g/min.

### (S)-N-(8,9-Difluoro-6-oxo-1,4,5,6-tetrahydro-2H-pyrano[3,4-c]isoquinolin-1-yl)-N-methylindolizine-6-carboxamide (Compound 227)

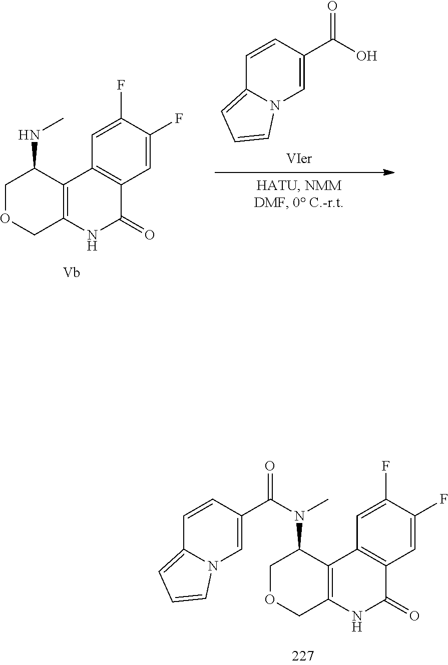

Enantiomerically pure (S)-N-(8,9-difluoro-6-oxo-1,4,5,6-tetrahydro-2H-pyrano[3,4-c]isoquinolin-1-yl)-N-methylindolizine-6-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and indolizine-6-carboxylic acid (VIer). LCMS m/z found 410.3 [M+H]⁺; RT=4.65 min (Method A); ¹H NMR (400 MHz, Methanol-d₄) δ 8.41 (s, 1H), 8.17 (dd, 1H), 7.57-7.52 (m, 2H), 7.42 (d, 1H), 6.82 (m, 1H), 6.66 (d, 1H), 6.47 (d, 1H), 5.78 (s, 1H), 4.67 (d, 1H), 4.56 (d, 1H), 4.36 (d, 1H), 4.11 (dd, 1H), 3.00 (s, 3H).

### (S)-N-(8,9-Difluoro-6-oxo-1,4,5,6-tetrahydro-2H-pyrano[3,4-c]isoquinolin-1-yl)-N-methylindolizine-7-carboxamide (Compound 228)

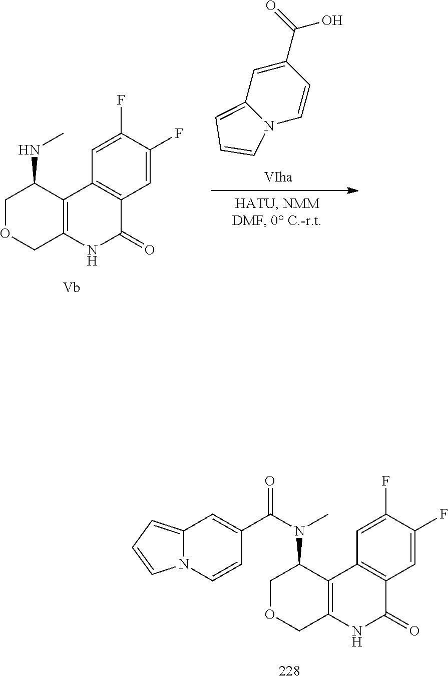

Enantiomerically pure (S)-N-(8,9-difluoro-6-oxo-1,4,5,6-tetrahydro-2H-pyrano[3,4-c]isoquinolin-1-yl)-N-methylindolizine-7-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and indolizine-7-carboxylic acid (VIha). LCMS m/z found 410.3 [M+H]⁺; RT=4.61 min (Method A); ¹H NMR (400 MHz, DMSO-d₆) δ 11.71 (s, 1H), 8.33-8.26 (m, 1H), 8.13 (m, 1H), 7.64 (d, 1H), 7.57 (s, 1H), 7.50 (s, 1H), 6.82 (m, 1H), 6.56 (m, 2H), 5.65 (s, 1H), 4.60 (d, 1H), 4.46 (d, 1H), 4.25 (d, 1H), 4.07-3.98 (m, 1H), 2.84 (s, 3H).

### (S)-N-(8,9-Difluoro-6-oxo-1,4,5,6-tetrahydro-2H-pyrano[3,4-c]isoquinolin-1-yl)-N-methyl-5,6,7,8-tetrahydroindolizine-2-carboxamide (Compound 242)

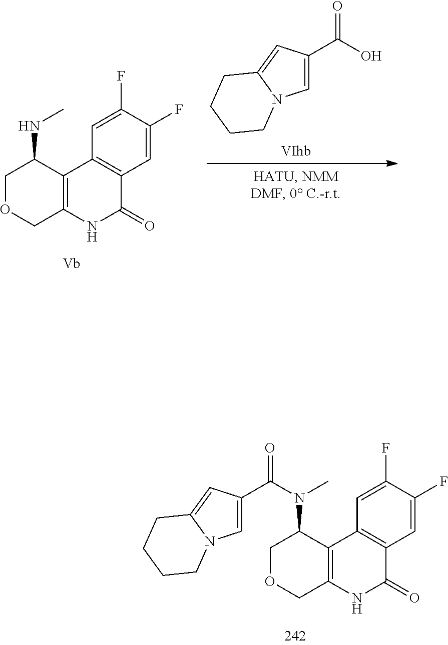

Enantiomerically pure (S)-N-(8,9-difluoro-6-oxo-1,4,5,6-tetrahydro-2H-pyrano[3,4-c]isoquinolin-1-yl)-N-methyl-5,6,7,8-tetrahydroindolizine-2-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5,6,7,8-tetrahydroindolizine-2-carboxylic acid (VIhb). LCMS m/z found 414.3 [M+H]⁺; RT=5.24 min (Method A); ¹H NMR (400 MHz, Chloroform-d) δ 11.50 (s, 1H), 8.19 (m, 1H), 7.49 (t, 1H), 7.02 (d, 1H), 6.14-6.09 (m, 1H), 5.86 (s, 1H), 4.77 (d, 1H), 4.61 (d, 1H), 4.34 (d, 1H), 4.03 (d, 1H), 3.95 (t, 2H), 3.10 (s, 3H), 2.75 (t, 2H), 2.00-1.90 (m, 2H), 1.82 (m, 2H).

### (S)-N-(8,9-Difluoro-6-oxo-1,4,5,6-tetrahydro-2H-pyrano[3,4-c]isoquinolin-1-yl)-N-methylindolizine-3-carboxamide (Compound 248)

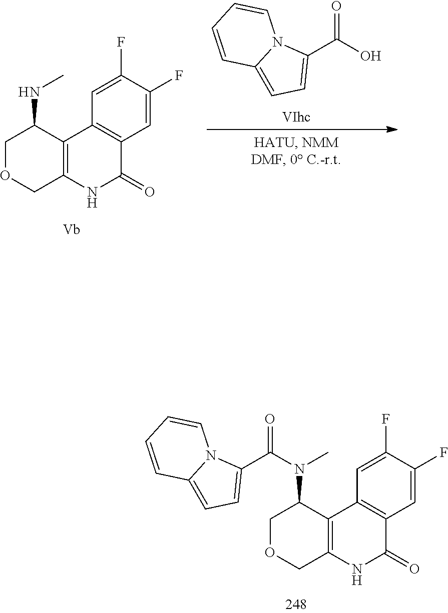

Enantiomerically pure (S)-N-(8,9-difluoro-6-oxo-1,4,5,6-tetrahydro-2H-pyrano[3,4-c]isoquinolin-1-yl)-N-methylindolizine-3-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and indolizine-3-carboxylic acid (VIhc). LCMS m/z found 410.3 [M+H]⁺; RT=5.89 min (Method A); ¹H NMR (400 MHz, Chloroform-d) δ 11.13 (s, 1H), 9.17 (m, 1H), 8.22 (m, 1H), 7.62 (s, 1H), 7.46 (m, 1H), 7.09 (d, 1H), 6.95 (m, 1H), 6.80-6.72 (m, 1H), 6.46 (m, 1H), 5.86 (s, 1H), 4.78 (d, 1H), 4.69-4.60 (m, 1H), 4.45 (d, 1H), 4.17-4.07 (m, 1H), 3.20 (s, 3H).

### (S)-N-(8,9-Difluoro-6-oxo-1,4,5,6-tetrahydro-2H-pyrano[3,4-c]isoquinolin-1-yl)-N-methylindolizine-1-carboxamide (Compound 261)

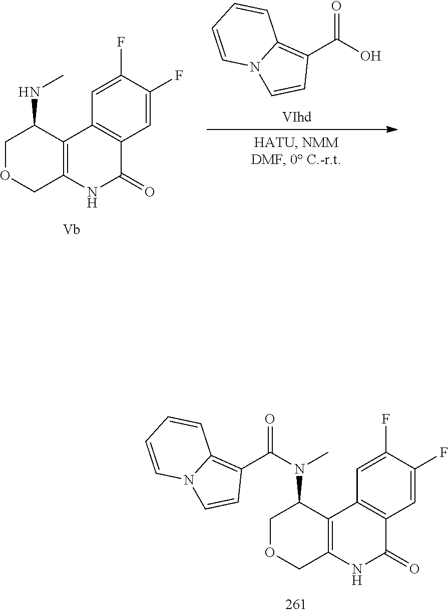

Enantiomerically pure (S)-N-(8,9-difluoro-6-oxo-1,4,5,6-tetrahydro-2H-pyrano[3,4-c]isoquinolin-1-yl)-N-methylindolizine-1-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and indolizine-1-carboxylic acid (VIhd). LCMS m/z found 410.3 [M+H]⁺; RT=5.67 min (Method A); ¹H NMR (400 MHz, Chloroform-d) δ 11.16 (s, 1H), 8.21 (m, 1H), 8.05-7.93 (m, 2H), 7.68 (s, 1H), 7.28-7.22 (m, 1H), 6.98 (m, 1H), 6.91 (d, 1H), 6.67 (m, 1H), 5.90 (s, 1H), 4.77 (d, 1H), 4.63 (d, 1H), 4.47 (d, 1H), 4.11 (m, 1H), 3.13 (s, 3H).

### (S)-N-(8,9-Difluoro-6-oxo-1,4,5,6-tetrahydro-2H-pyrano[3,4-c]isoquinolin-1-yl)-N,2-dimethylindolizine-6-carboxamide (Compound 277)

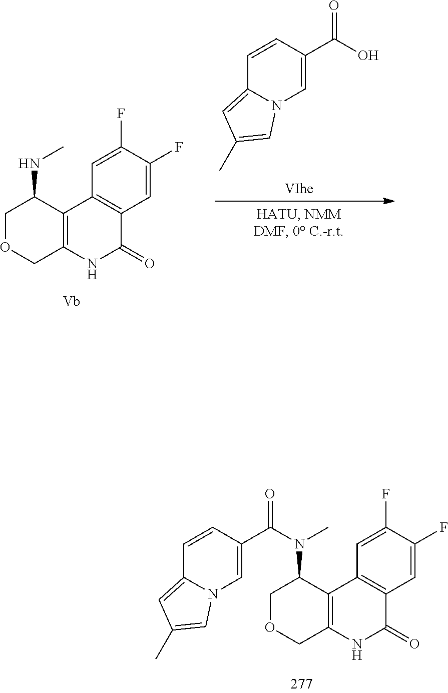 [SMILES: CN[C@@H]1COCc2[nH]c(=O)c3cc(F)c(F)cc3c21 ; Cc1cc2ccc(C(=O)O)cn2c1 ; Cc1cc2ccc(C(=O)N(C)[C@@H]3COCc4[nH]c(=O)c5cc(F)c(F)cc5c43)cn2c1]

Enantiomerically pure (S)-N-(8,9-difluoro-6-oxo-1,4,5,6-tetrahydro-2H-pyrano[3,4-c]isoquinolin-1-yl)-N,2-dimethylindolizine-6-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 2-methylindolizine-6-carboxylic acid (VIhe). LCMS m/z found 424 [M+H]⁺; RT=3.03 min (Method C); ¹H NMR (400 MHz, DMSO-d₆) δ 11.71 (s, 1H), 8.46 (s, 1H), 8.13 (m, 1H), 7.48 (s, 1H), 7.43 (s, 1H), 7.32 (d, 1H), 6.64 (d, 1H), 6.27 (s, 1H), 5.66-5.60 (m, 1H), 4.60 (d, 1H), 4.46 (d, 1H), 4.23 (d, 1H), 4.03 (m, 1H), 2.85 (s, 3H), 2.24 (s, 3H).

### (S)-N-(8,9-Difluoro-6-oxo-1,4,5,6-tetrahydro-2H-pyrano[3,4-c]isoquinolin-1-yl)-N-methyl-4H-thieno[3,2-b]pyrrole-5-carboxamide (Compound 278)

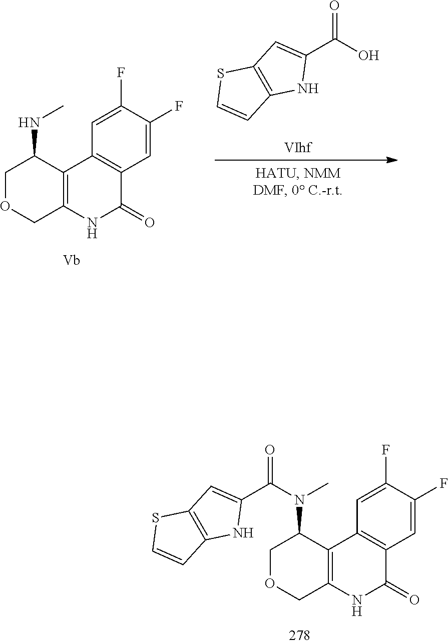

Enantiomerically pure (S)-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4H-thieno[3,2-b]pyrrole-5-carboxylic acid (VIhf). LCMS m/z found 416 [M+H]⁺; RT=2.87 min (Method C); ¹H NMR (400 MHz, DMSO-d₆) δ 11.86 (s, 1H), 11.73 (s, 1H), 8.12 (m, 1H), 7.44 (m, 2H), 7.03-6.95 (m, 2H), 5.74 (s, 1H), 4.64 (d, 1H), 4.52-4.42 (m, 1H), 4.12 (d, 1H), 4.03 (d, 1H), 3.13 (s, 3H).

### (S)-N-(8,9-Difluoro-6-oxo-1,4,5,6-tetrahydro-2H-pyrano[3,4-c]isoquinolin-1-yl)-N-methyl-6H-thieno[2,3-b]pyrrole-5-carboxamide (Compound 294)

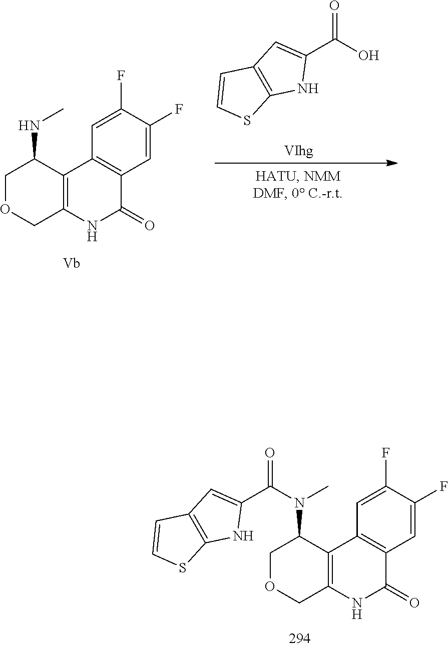

Enantiomerically pure (S)-N-(8,9-difluoro-6-oxo-1,4,5,6-tetrahydro-2H-pyrano[3,4-c]isoquinolin-1-yl)-N-methyl-6H-thieno[2,3-b]pyrrole-5-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 6H-thieno[2,3-b]pyrrole-5-carboxylic acid (VIhg). LCMS m/z found 416 [M+H]⁺; RT=2.89 min (Method C); ¹H NMR (400 MHz, DMSO-d₆) δ 12.05 (s, 1H), 11.73 (s, 1H), 8.12 (m, 1H), 7.45 (s, 1H), 7.07 (d, 1H), 6.99 (d, 1H), 6.87 (d, 1H), 5.74 (s, 1H), 4.63 (d, 1H), 4.51-4.42 (m, 1H), 4.12 (d, 1H), 4.02 (d, 1H), 3.13 (s, 3H).

### (S)-N-(8,9-Difluoro-6-oxo-1,4,5,6-tetrahydro-2H-pyrano[3,4-c]isoquinolin-1-yl)-N-methylimidazo[1,5-a]pyridine-6-carboxamide (Compound 299)

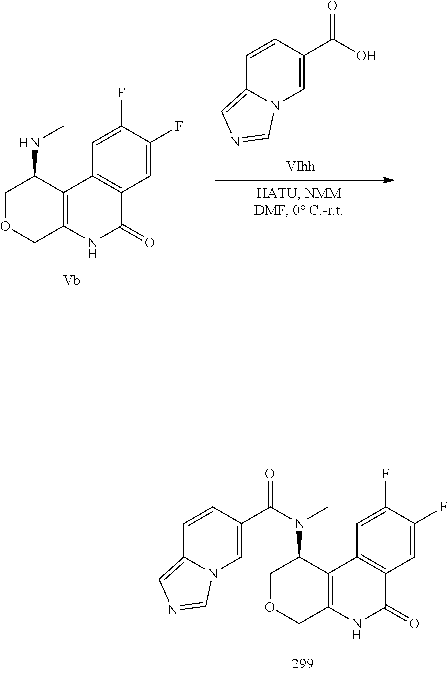

Enantiomerically pure (S)-N-(8,9-difluoro-6-oxo-1,4,5,6-tetrahydro-2H-pyrano[3,4-c]isoquinolin-1-yl)-N-methylimidazo[1,5-a]pyridine-6-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and imidazo[1,5-a]pyridine-6-carboxylic acid (VIhh). LCMS m/z found 411 [M+H]⁺; RT=2.02 min (Method C); ¹H NMR (400 MHz, DMSO-d₆) δ 11.73 (s, 1H), 8.64-8.59 (m, 1H), 8.43 (s, 1H), 8.14 (m, 1H), 7.61 (d, 1H), 7.48 (m, 1H), 7.41 (s, 1H), 6.78 (d, 1H), 5.65 (s, 1H), 4.60 (d, 1H), 4.47 (d, 1H), 4.26 (d, 1H), 4.03 (m, 1H), 2.85 (s, 3H).

### (S)-N-(8,9-Difluoro-6-oxo-1,4,5,6-tetrahydro-2H-pyrano[3,4-c]isoquinolin-1-yl)-N-methylimidazo[1,2-a]pyridine-6-carboxamide (Compound 306)

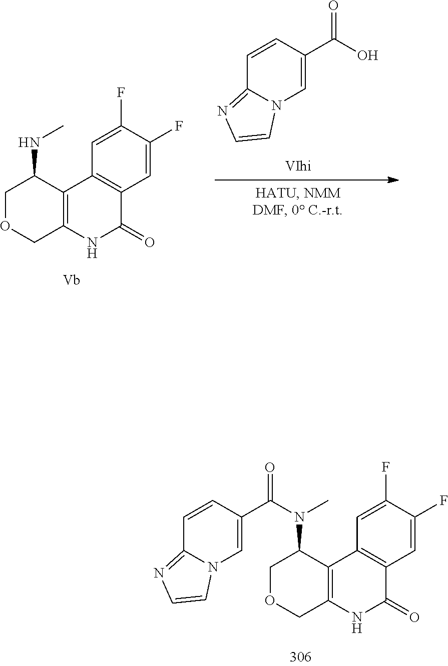

Enantiomerically pure (S)-N-(8,9-difluoro-6-oxo-1,4,5,6-tetrahydro-2H-pyrano[3,4-c]isoquinolin-1-yl)-N-methylimidazo[1,2-a]pyridine-6-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and imidazo[1,2-a]pyridine-6-carboxylic acid (VIhi). LCMS m/z found 411 [M+H]⁺; RT=1.80 min (Method C); ¹H NMR (400 MHz, DMSO-d₆) δ 11.73 (s, 1H), 8.86 (s, 1H), 8.19-8.09 (m, 1H), 8.02 (s, 1H), 7.68-7.59 (m, 2H), 7.51 (m, 1H), 7.28 (d, 1H), 5.67 (s, 1H), 4.61 (d, 1H), 4.47 (d, 1H), 4.28 (d, 1H), 4.08-4.00 (m, 1H), 2.85 (s, 3H).

### (S)-N-(8,9-Difluoro-6-oxo-1,4,5,6-tetrahydro-2H-pyrano[3,4-c]isoquinolin-1-yl)-N-methyl-2-(trifluoromethyl)indolizine-6-carboxamide (Compound 311)

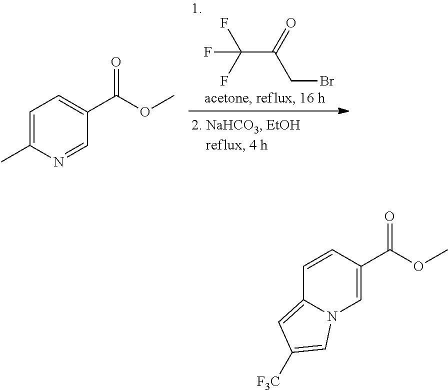

Step i. A mixture of methyl 6-methylpyridine-3-carboxylate (1.00 g, 6.62 mmol) and 3-bromo-1,1,1-trifluoro-propan-2-one (0.81 mL, 7.80 mmol) in 3 mL acetone was heated to reflux overnight. The volatiles were evaporated, and the material was triturated with 5% ethyl acetate in hexanes, dried over sodium sulfate and carried forward crude. Sodium bicarbonate (0.98 g, 11.7 mmol) was added to a solution of crude methyl 6-methyl-1-(3,3,3-trifluoro-2-oxo-propyl)pyridin-1-ium-3-carboxylate bromide (2.00 g, 5.85 mmol) in 10 mL ethanol. The mixture was heated to reflux. After refluxing for 4 h, the mixture was stirred at room temperature overnight. The mixture was filtered through a cotton plug and evaporated to dryness. The resulting dark oil was taken up in 50 mL DCM and washed with water (25 mL) and brine (25 mL). The organics were dried over sodium sulfate and evaporated under reduced pressure. The material was isolated by flash chromatography (silica gel, EtOAc/Hexane 5-30%) to provide methyl 2-(trifluoromethyl)indolizine-6-carboxylate (0.18 g, 13%). ¹H NMR (400 MHz, Chloroform-d) δ 8.72 (dd, 1H), 7.66 (d, 1H), 7.44-7.37 (m, 1H), 7.33-7.19 (m, 1H), 6.66 (s, 1H), 3.94 (s, 3H).

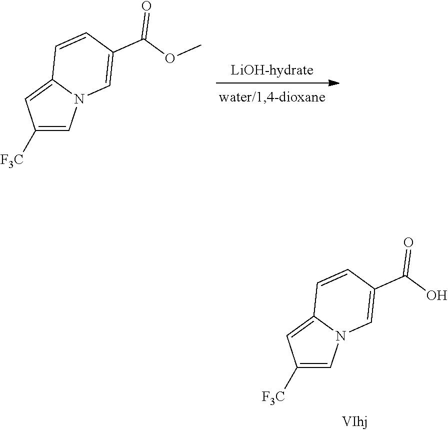 [SMILES: COC(=O)c1ccc2cc(C(F)(F)F)cn2c1 ; O=C(O)c1ccc2cc(C(F)(F)F)cn2c1]

Step ii: A solution of lithium hydroxide hydrate (35 mg, 0.82 mmol) in 2 mL water was added slowly to a solution of methyl 2-(trifluoromethyl)indolizine-6-carboxylate (100 mg, 0.41 mmol) in 3 mL 1,4-dioxane. After stirring overnight at room temperature, the volatiles were removed in vacuo. The residue was acidified to pH=1 with 2 N HCl. The resulting precipitate was collected by vacuum filtration, and dried in vacuo to provide 2-(trifluoromethyl)indolizine-6-carboxylic acid (VIhj, 75 mg, 80%). ¹H NMR (400 MHz, DMSO-d₆) δ 13.19 (s, 1H), 9.10 (m, 1H), 8.26 (t, 1H), 7.56 (d, 1H), 7.22 (m, 1H), 6.81 (s, 1H).

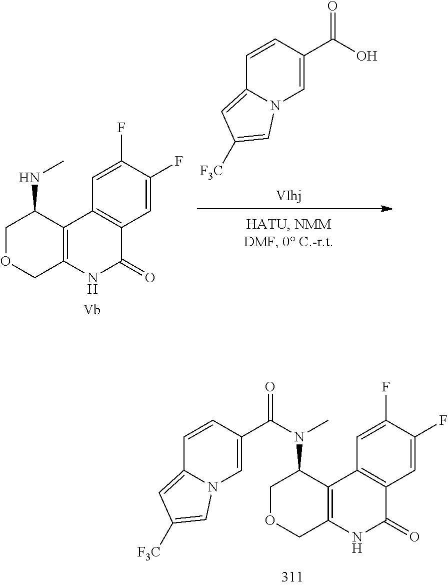

Step iii: Enantiomerically pure (S)-N-(8,9-difluoro-6-oxo-1,4,5,6-tetrahydro-2H-pyrano[3,4-c]isoquinolin-1-yl)-N-methyl-2-(trifluoromethyl)indolizine-6-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) 2-(trifluoromethyl)indolizine-6-carboxylic acid (VIhj). LCMS m/z found 478 [M+H]⁺; RT=3.27 min (Method C); ¹H NMR (400 MHz, DMSO-d₆) δ 11.72 (s, 1H), 8.61 (s, 1H), 8.14 (m, 2H), 7.58 (d, 1H), 7.48 (m, 1H), 6.89 (d, 1H), 6.78 (s, 1H), 5.68-5.62 (m, 1H), 4.61 (d, 1H), 4.47 (d, 1H), 4.26 (d, 1H), 4.03 (m, 1H), 2.85 (s, 3H).

### (S)-N-(8,9-Difluoro-6-oxo-1,4,5,6-tetrahydro-2H-pyrano[3,4-c]isoquinolin-1-yl)-N-methyl-4H-furo[3,2-b]pyrrole-5-carboxamide (Compound 314)

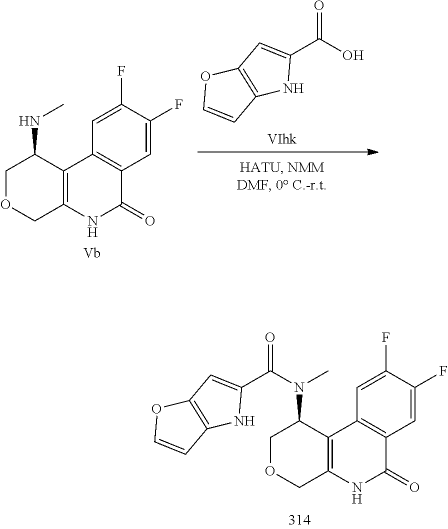

Enantiomerically pure (S)-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 4H-furo[3,2-b]pyrrole-5-carboxylic acid (VIhk). LCMS m/z found 400.3 [M+H]⁺; RT=3.45 min (Method A); ¹H NMR (400 MHz, DMSO-d₆) δ 11.72 (s, 1H), 11.45 (s, 1H), 8.11 (m, 1H), 7.73 (d, 1H), 7.45 (s, 1H), 6.68 (s, 1H), 6.59 (d, 1H), 5.72 (s, 1H), 4.63 (d, 1H), 4.46 (d, 1H), 4.11 (d, 1H), 4.02 (d, 1H), 3.09 (s, 3H).

### (S)-2-Chloro-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide (Compound 332)

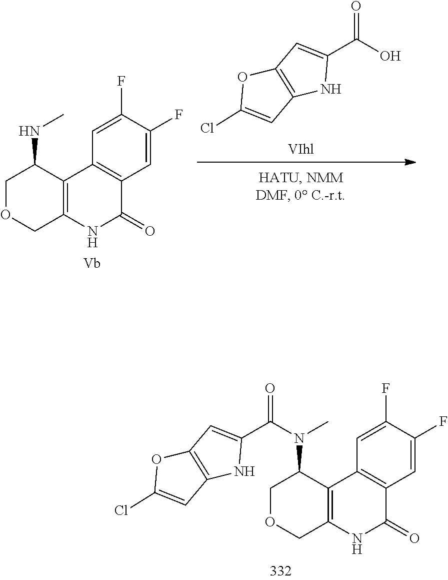

Enantiomerically pure (S)-2-chloro-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 2-chloro-4H-furo[3,2-b]pyrrole-5-carboxylic acid (VIhl). LCMS m/z found 434.2/436.2 [M+H]⁺; RT=4.18 min (Method A); ¹H NMR (400 MHz, DMSO-d₆) δ 11.71 (s, 1H), 11.63 (s, 1H), 8.10 (m, 1H), 7.42 (s, 1H), 6.70 (s, 2H), 5.69 (s, 1H), 4.61 (d, 1H), 4.44 (d, 1H), 4.09 (d, 1H), 3.99 (d, 1H), 3.06 (s, 3H).

### (S)-2-Chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide (Compound 349)

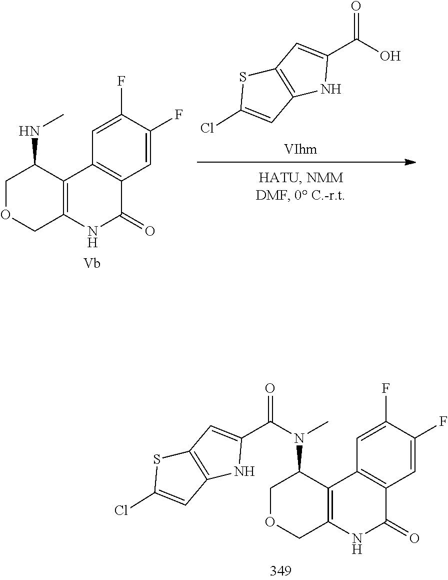

Enantiomerically pure (S)-2-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 2-chloro-4H-thieno[3,2-b]pyrrole-5-carboxylic acid (VIhm). LCMS m/z found 450.1/452.0 [M+H]⁺; RT=4.56 min (Method A); ¹H NMR (400 MHz, DMSO-d₆) δ 12.01 (s, 1H), 11.73 (s, 1H), 8.12 (m, 1H), 7.43 (d, 1H), 7.09 (s, 1H), 6.96-6.91 (m, 1H), 5.73 (s, 1H), 4.63 (d, 1H), 4.47 (d, 1H), 4.12 (d, 1H), 4.02 (d, 1H), 3.11 (s, 3H).

### (S)-3-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide (Compound 350)

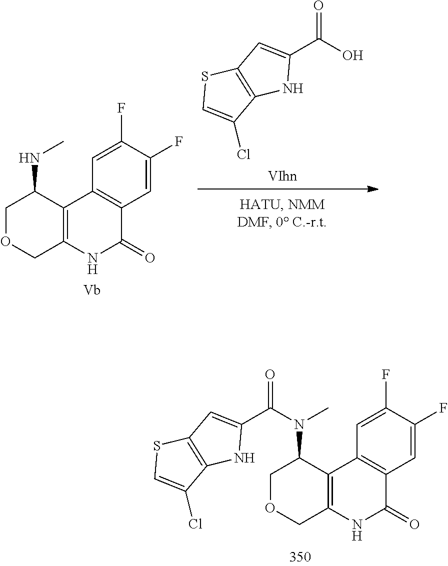

Enantiomerically pure (S)-3-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-chloro-4H-thieno[3,2-b]pyrrole-5-carboxylic acid (VIhn). LCMS m/z found 450.0 [M+H]⁺; RT=4.33 min (Method A); ¹H NMR (400 MHz, DMSO-d₆) δ 12.35 (s, 1H), 11.74 (s, 1H), 8.12 (m, 1H), 7.46 (m, 2H), 6.98 (s, 1H), 5.71 (s, 1H), 4.63 (d, 1H), 4.47 (d, 1H), 4.14 (d, 1H), 4.07-3.99 (m, 1H), 3.10 (s, 3H).

### (S)-3,4-Dichloro-N-(8,9-difluoro-6-oxo-1,4,5,6-tetrahydro-2H-pyrano[3,4-c]isoquinolin-1-yl)-N-methylbenzamide (Compound 361)

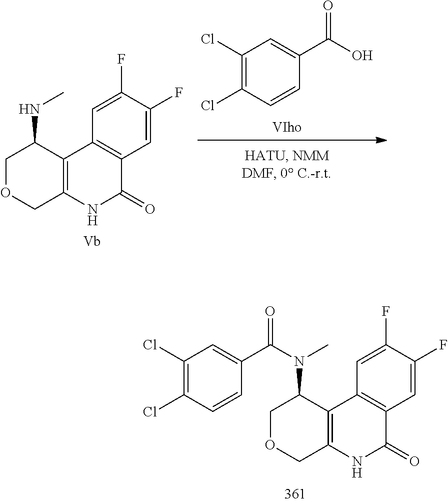

Enantiomerically pure ((S)-3,4-dichloro-N-(8,9-difluoro-6-oxo-1,4,5,6-tetrahydro-2H-pyrano[3,4-c]isoquinolin-1-yl)-N-methylbenzamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3,4-dichlorobenzoic acid (VIho). LCMS m/z found 439.1 [M+H]⁺; RT=4.56 min (Method A); ¹H NMR (400 MHz, DMSO-d₆) δ 11.71 (s, 1H), 8.13 (m, 1H), 7.78-7.69 (m, 2H), 7.47 (m, 1H), 7.38 (m, 1H), 5.65 (d, 1H), 4.59 (d, 1H), 4.46 (d, 1H), 4.31 (d, 1H), 4.00 (m, 1H), 2.70 (s, 3H).

### (S)-3-Chloro-N-(8,9-difluoro-6-oxo-1,4,5,6-tetrahydro-2H-pyrano[3,4-c]isoquinolin-1-yl)-N-methylindolizine-6-carboxamide (Compound 366)

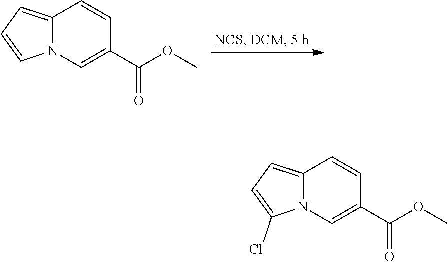

Step i. A suspension of N-chlorosuccinimide (34 mg, 0.25 mmol) in DCM (0.30 mL) was added to a 0° C. solution of methyl indolizine-6-carboxylate (40 mg, 0.23 mmol) in 0.2 mL DCM (0.2 mL) in a foil-wrapped flask. After 30 minutes, the reaction mixture was diluted with 15 mL EtOAc, and washed with saturated sodium thiosulfate (2×3 mL) and 3 mL brine. The organics were dried over sodium sulfate and evaporated to dryness. Purification by flash-chromatography (silicagel, DCM/hexanes 20-80%) provided pure methyl 3-chloroindolizine-6-carboxylate (36 mg, 76%). ¹H NMR (400 MHz, Chloroform-d) δ 8.75 (dt, 1H), 7.37 (dd, 1H), 7.29-7.18 (m, 1H), 6.81 (dd, 1H), 6.51 (dd, 1H), 3.94 (s, 3H).

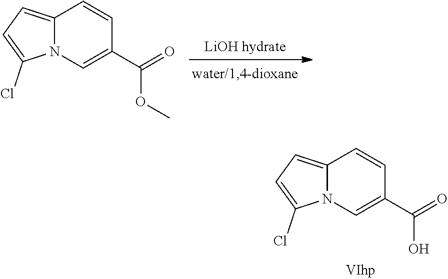

Step ii: A solution of lithium hydroxide hydrate (10 mg, 0.23 mmol) in 0.6 mL water was added to a solution of methyl 3-chloroindolizine-6-carboxylate (32 mg, 0.15 mmol) in 0.5 mL 1,4-dioxane. After stirring at room temperature for 150 minutes, the mixture was acidified to pH=1 with 2N HCl. The mixture was extracted with EtOAc (3×4 mL). The combined organics were dried over sodium sulfate, filtered, and rotovaced to provide 3-chloroindolizine-6-carboxylic acid (VIhp, 29 mg, 97.5%) as a yellow solid. ¹H NMR (400 MHz, Methanol-d₄) δ 8.75 (m, 1H), 7.46 (m, 1H), 7.22 (dd, 1H), 6.87 (dd, 1H), 6.58 (d, 1H).

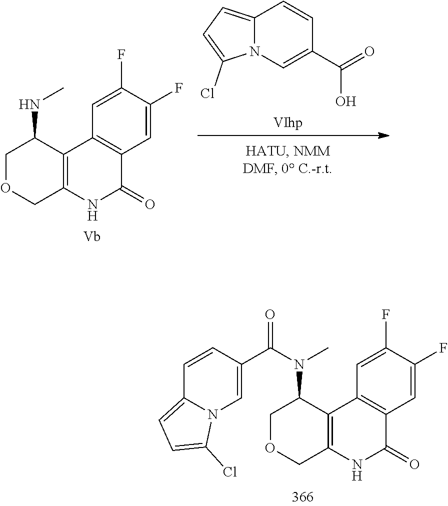

Step iii: Enantiomerically pure (S)-3-chloro-N-(8,9-difluoro-6-oxo-1,4,5,6-tetrahydro-2H-pyrano[3,4-c]isoquinolin-1-yl)-N-methylindolizine-6-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-chloroindolizine-6-carboxylic acid (VIhp). LCMS m/z found 444.1/446.1 [M+H]⁺; RT=4.46 min (Method A); ¹H NMR (400 MHz, DMSO-d₆) δ 11.72 (s, 1H), 8.26 (s, 1H), 8.18-8.08 (m, 1H), 7.61-7.49 (m, 2H), 6.96 (d, 1H), 6.78-6.71 (m, 1H), 6.62 (d, 1H), 5.66 (s, 1H), 4.60 (d, 1H), 4.47 (d, 1H), 4.35 (d, 1H), 4.06-3.97 (m, 1H), 2.82 (s, 3H).

### (S)-3-Chloro-N-(8,9-difluoro-6-oxo-1,4,5,6-tetrahydro-2H-pyrano[3,4-c]isoquinolin-1-yl)-N-methylindolizine-7-carboxamide (Compound 373)

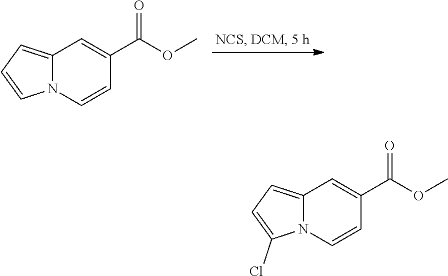

Step i: N-Chlorosuccinimide (105 mg, 0.78 mmol) was added in 3 portions to a 0° C. solution of methyl indolizine-7-carboxylate (125 mg, 0.71 mmol) in 0.2 mL DCM in a foil wrapped flask. After 5 h at 0° C., the reaction mixture was diluted with 15 mL EtOAc, and washed with saturated sodium thiosulfate, (2×3 mL), and 3 mL brine. The organics were dried over sodium sulfate and evaporated to dryness. Purification by flash-chromatography (silicagel, DCM/hexanes 0-50%) provided pure methyl 3-chloroindolizine-7-carboxylate (VIhq, 72 mg, 48%). NMR (400 MHz, Chloroform-d) δ 8.19 (m, 1H), 7.92 (m, 1H), 7.30-7.17 (m, 1H), 6.82 (m, 1H), 6.78-6.72 (m, 1H), 3.92 (s 3H).

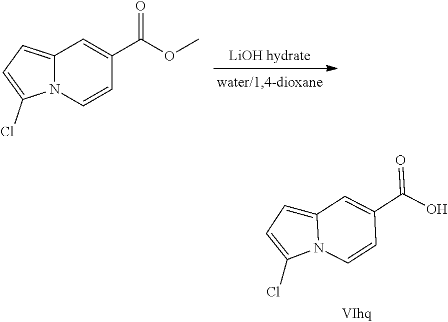

Step ii: A solution of lithium hydroxide hydrate (17 mg, 0.40 mmol) in 1 mL water was added to a solution of methyl 3-chloroindolizine-7-carboxylate (56 mg, 0.27 mmol) in 1 mL 1,4-dioxane. After 3 h, the mixture was acidified to pH=1 with 2N HCl. The mixture was extracted with EtOAc (3×4 mL). The combined organics were dried over sodium sulfate, filtered, and evaporated to provide 3-chloroindolizine-7-carboxylic acid (45 mg, 86%). ¹H NMR (400 MHz, DMSO-d₆) δ 12.93 (s, 1H), 8.23-8.10 (m, 2H), 7.16 (m, 1H), 7.03 (dd, 1H), 6.94-6.86 (m, 1H).

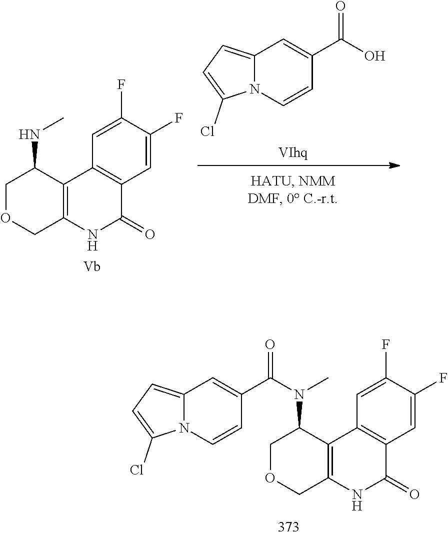

Step iii: Enantiomerically pure (S)-3-chloro-N-(8,9-difluoro-6-oxo-1,4,5,6-tetrahydro-2H-pyrano[3,4-c]isoquinolin-1-yl)-N-methylindolizine-7-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 3-chloroindolizine-7-carboxylic acid (VIhq). LCMS m/z found 444.2/446.1 [M+H]⁺; RT=4.48 min (Method A); ¹H NMR (400 MHz, DMSO-d₆) δ 11.71 (s, 1H), 8.18-8.11 (m, 2H), 7.69 (s, 1H), 7.50 (t, 1H), 6.95 (d, 1H), 6.85-6.77 (m, 1H), 6.71 (d, 1H), 5.65 (s, 1H), 4.60 (d, 1H), 4.47 (d, 1H), 4.27 (d, 1H), 4.07-3.98 (m, 1H), 2.84 (s, 3H).

### ((S)-2-Cyano-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide (Compound 468)

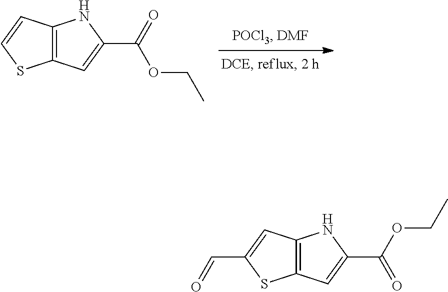

Step i. Phosphorus (V) oxychloride (0.48 mL, 5.13 mmol) was added dropwise to a solution of N,N-dimethylformamide (0.60 mL, 7.70 mmol) in 10 mL 1,2-dichloroethane. After 30 minutes, a solution of ethyl 4H-thieno[3,2-b]pyrrole-5-carboxylate (0.50 g, 2.57 mmol) in 14 mL 1,2-dichloroethane was added. The mixture was heated to reflux for 2 h. After cooling, the mixture was slowly poured into 20 mL water, then evaporated under reduced pressure to remove 1,2-dichloroethane. The mixture was extracted with EtOAc (3×25 mL). The combined organics were washed with saturated aqueous sodium bicarbonate (2×20 mL) and 20 mL brine, and dried over sodium sulfate. The product was isolated by flash-chromatography (silicagel, EtOAc/DCM 0-40%) to provide ethyl 6-formyl-4H-thieno[3,2-b]pyrrole-5-carboxylate (198 mg, 35%) as the first eluting regioisomer and ethyl 2-formyl-4H-thieno[3,2-b]pyrrole-5-carboxylate (162 mg, 28%) as the second eluting regioisomer. Ethyl 2-formyl-4H-thieno[3,2-b]pyrrole-5-carboxylate: ¹H NMR (400 MHz, Chloroform-d) δ 9.93 (s, 1H), 9.29 (s, 1H), 7.64 (d, 1H), 7.16 (m, 1H), 4.41 (q, 2H), 1.41 (t, 3H).

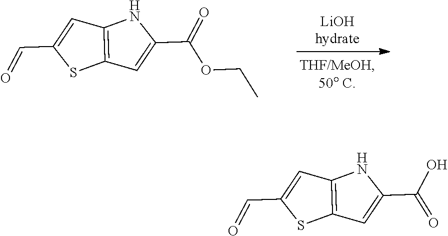

Step ii: A solution was prepared of ethyl 2-formyl-4H-thieno[3,2-b]pyrrole-5-carboxylate (160 mg, 0.73 mmol) in 3.4 mL THE and 2.3 mL MeOH. A solution of lithium hydroxide hydrate (122 mL, 2.90 mmol) in 1.2 mL water was added. The mixture was heated to 50° C. overnight. The volatiles were removed in vacuo. The aqueous residue was acidified to pH=1 with 2 N HCl. A tan precipitate formed. The precipitate was collected by vacuum filtration and dried in vacuo to provide 2-formyl-4H-thieno[3,2-b]pyrrole-5-carboxylic acid (70 mg). The filtrate was extracted with 30 mL EtOAc. The organic layer was separated, and the aqueous layer was extracted with 40 mL EtOAc. The combined organics were dried over sodium sulfate, filtered, rotovaced, and dried in vacuo. The material was combined with the above product to provide crude 2-formyl-4H-thieno[3,2-b]pyrrole-5-carboxylic acid (145 mg). ¹H NMR (400 MHz, DMSO-d₆) δ 13.13 (s, 1H), 12.47 (s, 1H), 9.94 (s, 1H), 7.95 (d, 1H), 7.12 (m, 1H).

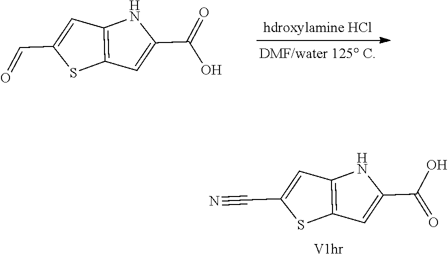 [SMILES: O=Cc1cc2[nH]c(C(=O)O)cc2s1 ; N#Cc1cc2[nH]c(C(=O)O)cc2s1]

Step iii: A mixture of crude 2-formyl-4H-thieno[3,2-b]pyrrole-5-carboxylic acid (75 mg, 0.38 mmol) and hydroxylamine hydrochloride (59 mg, 0.85 mmol) in 2M anhydrous DMF was heated to 125° C. overnight. The volatiles were removed in vacuo. The resulting residue was suspended in 15 mL water and 15 mL EtOAc, and the layers separated. The aqueous layer was extracted with 2×15 mL EtOAc. The combined organics were washed with brine (10 mL), dried over sodium sulfate, evaporated to dryness and dried under high vacuum to provide crude 2-cyano-4H-thieno[3,2-b]pyrrole-5-carboxylic acid (VIhr, 72 mg). ¹H NMR (400 MHz, DMSO-d₆) δ 13.13 (s, 1H), 12.52-12.46 (m, 1H), 7.88 (s, 1H), 7.16-7.10 (m, 1H).

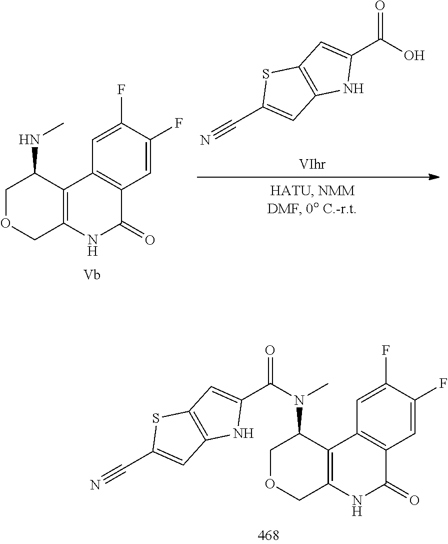

Step iv: Enantiomerically pure (S)-2-cyano-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 2-cyano-4H-thieno[3,2-b]pyrrole-5-carboxylic acid (VIhr). LCMS m/z found 441.2 [M+H]⁺; RT=3.83 min (Method A); ¹H NMR (400 MHz, DMSO-d₆) δ 12.41 (s, 1H), 11.74 (s, 1H), 8.17-8.09 (m, 1H), 7.88 (s, 1H), 7.47-7.39 (m, 1H), 7.05 (s, 1H), 5.71 (s, 1H), 4.63 (d, 1H), 4.47 (d, 1H), 4.16 (d, 1H), 4.07-3.98 (m, 1H), 3.11 (s, 3H).

### N-(8-Fluoro-6-oxo-1,4,5,6-tetrahydro-2H-pyrano[3,4-c]isoquinolin-1-yl)-N-methyl-2,3-dihydro-1H-indene-5-carboxamide (Compounds 442 and 443)

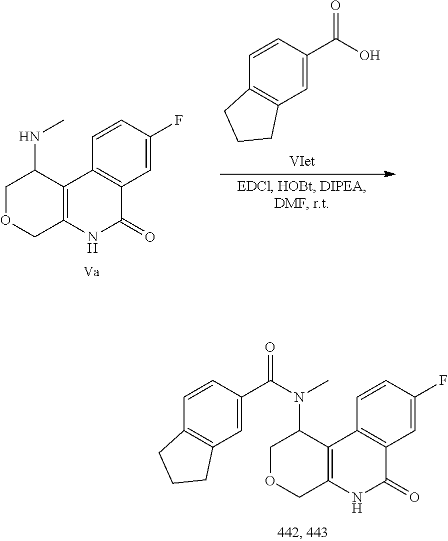

Racemic N-(8-fluoro-6-oxo-1,4,5,6-tetrahydro-2H-pyrano[3,4-c]isoquinolin-1-yl)-N-methyl-2,3-dihydro-1H-indene-5-carboxamide was synthesized in an analogous manner as described above from racemic 8-fluoro-1-(methylamino)-1,5-dihydro-2H-pyrano[3,4-c]isoquinolin-6(4H)-one (Va) and 2,3-dihydro-1H-indene-5-carboxylic acid (VIet). The enantiomers were subsequently separated by preparative SFC: Method isocratic, Mobile phase methanol:CO₂—40:60. Column: Chiralcel-IG-H (30×250 mm), 5 μm, flow rate: 100 g/min.

Enantiomer I (Compound 442): LCMS: m/z found 393.1 [M+H]⁺, RT=4.09 min, (Method A); ¹H NMR (400 MHz, DMSO-d₆): δ 11.51 (bs, 1H), 7.91 (d, 1H), 7.78 (t, 1H), 7.67 (m, 1H), 7.28 (m, 2H), 7.13 (d, 1H), 5.68 (s, 1H), 4.59 (d, 1H), 4.48 (d, 1H), 4.22 (d, 1H), 4.04 (d, 1H), 2.88 (t, 4H), 2.67 (s, 3H) 2.03 (m, 2H); Chiral analytical SFC: RT=2.02 min, Column: Chiralcel IG-3 (4.6×150 mm) 3 μm, 50% of Methanol, Flow rate: 3.0 g/min.

Enantiomer II (Compound 443): LCMS: m/z found 393.1 [M+H]⁺, RT=4.10 min, (Method A); ¹H NMR (400 MHz, DMSO-d₆): δ 11.51 (bs, 1H), 7.91 (d, 1H), 7.78 (t, 1H), 7.67 (m, 1H), 7.28 (m, 2H), 7.13 (d, 1H), 5.68 (s, 1H), 4.59 (d, 1H), 4.48 (d, 1H), 4.22 (d, 1H), 4.04 (d, 1H), 2.88 (t, 4H), 2.67 (s, 3H) 2.03 (m, 2H); Chiral analytical SFC: RT=5.40 min, Column: Chiralcel IG-3 (4.6×150 mm) 3 μm, 45% of Methanol, Flow rate: 3.0 g/min.

### N-(8-fluoro-6-oxo-1,4,5,6-tetrahydro-2H-pyrano[3,4-c]isoquinolin-1-yl)-N-methylbenzo[d]thiazole-5-carboxamide (Compounds 444 and 445)

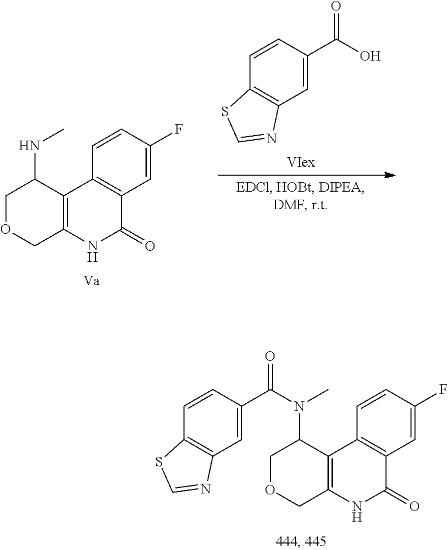 [SMILES: CNC1COCc2[nH]c(=O)c3cc(F)ccc3c21 ; O=C(O)c1ccc2scnc2c1 ; CN(C(=O)c1ccc2scnc2c1)C1COCc2[nH]c(=O)c3cc(F)ccc3c21]

Racemic N-(8-fluoro-6-oxo-1,4,5,6-tetrahydro-2H-pyrano[3,4-c]isoquinolin-1-yl)-N-methylbenzo[d]thiazole-5-carboxamide was synthesized in an analogous manner as described above from racemic 8-fluoro-1-(methylamino)-1,5-dihydro-2H-pyrano[3,4-c]isoquinolin-6(4H)-one (Va) and benzo[d]thiazole-5-carboxylic acid (VIex). The enantiomers were subsequently separated by preparative SFC: Method isocratic, Mobile phase methanol:CO₂—40:60. Column: Chiralcel-IG (30×250 mm), 5 μm, flow rate: 100 g/min.

Enantiomer I (Compound 444): LCMS: m/z found 410.1 [M+H]⁺, RT=2.94 min, (Method A); ¹H NMR (400 MHz, DMSO-d₆): δ 11.61 (bs, 1H), 9.48 (bs, 1H), 8.27 (d, 1H), 8.10 (bs, 1H), 7.93 (d, 1H), 7.83 (t, 1H), 7.71 (m, 1H), 7.51 (d, 1H), 5.75 (s, 1H), 4.61 (d, 1H), 4.50 (d, 1H), 4.33 (d, 1H), 4.08 (d, 1H), 2.71 (s, 3H); Chiral analytical SFC: RT=4.01 min, Column: Chiralcel IG-3 (4.6×150 mm) 3 μm, 40% of Methanol/Acetonitrile (1:1), Flow rate: 3.0 g/min.

Enantiomer II (Compound 445): LCMS: m/z found 410.1 [M+H]⁺, RT=2.94 min, (Method A); ¹H NMR (400 MHz, DMSO-d₆): δ 11.61 (bs, 1H), 9.48 (bs, 1H), 8.27 (d, 1H), 8.10 (bs, 1H), 7.93 (d, 1H), 7.83 (t, 1H), 7.71 (m, 1H), 7.51 (d, 1H), 5.75 (s, 1H), 4.61 (d, 1H), 4.50 (d, 1H), 4.33 (d, 1H), 4.08 (d, 1H), 2.71 (s, 3H); Chiral analytical SFC: RT=8.06 min, Column: Chiralcel IG-3 (4.6×150 mm) 3 μm, 40% of Methanol/Acetonitrile (1:1), Flow rate: 3.0 g/min.

### N-(8-fluoro-6-oxo-1,4,5,6-tetrahydro-2H-pyrano[3,4-c]isoquinolin-1-yl)-N-methylbenzo[d]thiazole-6-carboxamide (Compounds 446 and 447)

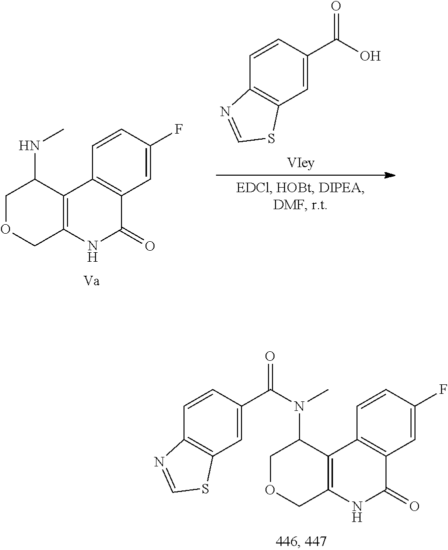 [SMILES: CNC1COCc2[nH]c(=O)c3cc(F)ccc3c21 ; O=C(O)c1ccc2ncsc2c1 ; CN(C(=O)c1ccc2ncsc2c1)C1COCc2[nH]c(=O)c3cc(F)ccc3c21]

Racemic N-(8-fluoro-6-oxo-1,4,5,6-tetrahydro-2H-pyrano[3,4-c]isoquinolin-1-yl)-N-methylbenzo[d]thiazole-6-carboxamide was synthesized in an analogous manner as described above from racemic 8-fluoro-1-(methylamino)-1,5-dihydro-2H-pyrano[3,4-c]isoquinolin-6(4H)-one (Va) and benzo[d]thiazole-6-carboxylic acid (VIey). The enantiomers were subsequently separated by preparative SFC: Method isocratic, Mobile phase methanol:CO₂—60:40. Column: Chiralcel-IG (30×250 mm), 5 μm, flow rate: 100 g/min.

Enantiomer I (Compound 446): LCMS: m/z found 410.1 [M+H]⁺, RT=2.82 min, (Method A); ¹H NMR (400 MHz, DMSO-d₆): δ 11.60 (bs, 1H), 9.48 (bs, 1H), 8.28 (bs, 1H), 8.15 (d, 1H), 7.93 (d, 1H), 7.80 (t, 1H), 7.69 (m, 1H), 7.57 (d, 1H), 5.72 (s, 1H), 4.61 (d, 1H), 4.50 (d, 1H), 4.29 (d, 1H), 4.08 (d, 1H), 2.72 (s, 3H); Chiral analytical SFC: RT=2.38 min, Column: Chiralcel IG-3 (4.6×150 mm) 3 μm, 40% of Methanol/Acetonitrile (1:1), Flow rate: 3.0 g/min.

Enantiomer II (Compound 447): LCMS: m/z found 410.1 [M+H]⁺, RT=2.82 min, (Method A); ¹H NMR (400 MHz, DMSO-d₆): δ 11.60 (bs, 1H), 9.48 (bs, 1H), 8.28 (bs, 1H), 8.15 (d, 1H), 7.93 (d, 1H), 7.80 (t, 1H), 7.69 (m, 1H), 7.57 (d, 1H), 5.72 (s, 1H), 4.61 (d, 1H), 4.50 (d, 1H), 4.29 (d, 1H), 4.08 (d, 1H), 2.71 (s, 3H); Chiral analytical SFC: RT=5.38 min, Column: Chiralcel IG-3 (4.6×150 mm) 3 μm, 40% of Methanol/Acetonitrile (1:1), Flow rate: 3.0 g/min.

### N-(8-fluoro-6-oxo-1,4,5,6-tetrahydro-2H-pyrano[3,4-c]isoquinolin-1-yl)-N-methyl-1H-indazole-5-carboxamide (Compounds 448 and 449)

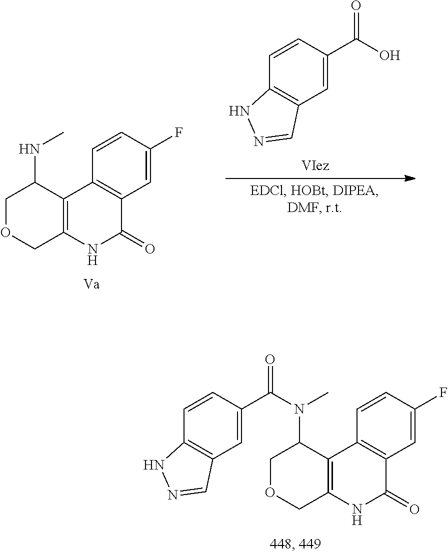 [SMILES: CNC1COCc2[nH]c(=O)c3cc(F)ccc3c21 ; O=C(O)c1ccc2[nH]ncc2c1 ; CN(C(=O)c1ccc2[nH]ncc2c1)C1COCc2[nH]c(=O)c3cc(F)ccc3c21]

Racemic N-(8-fluoro-6-oxo-1,4,5,6-tetrahydro-2H-pyrano[3,4-c]isoquinolin-1-yl)-N-methyl-1H-indazole-5-carboxamide was synthesized in an analogous manner as described above from racemic 8-fluoro-1-(methylamino)-1,5-dihydro-2H-pyrano[3,4-c]isoquinolin-6(4H)-one (Va) and 1H-indazole-5-carboxylic acid (VIez). The enantiomers were subsequently separated by preparative SFC: Method isocratic, Mobile phase methanol:CO₂—40:60. Column: Chiralcel-IG (30×250 mm), 5 μm, flow rate: 100 g/min.

Enantiomer I (Compound 448): LCMS: m/z found 393.1 [M+H]⁺, RT=2.35 min, (Method A); ¹H NMR (400 MHz, DMSO-d₆): δ 13.23 (bs, 1H), 11.60 (bs, 1H), 8.13 (bs, 1H), 7.93 (d, 1H), 7.84 (s, 1H), 7.79 (t, 1H), 7.69 (t, 1H), 7.60 (d, 1H), 7.39 (d, 1H), 5.72 (s, 1H), 4.60 (d, 1H), 4.49 (d, 1H), 4.27 (d, 1H), 4.07 (d, 1H), 2.74 (s, 3H); Chiral analytical SFC: RT=2.19 min, Column: Chiralcel IG-3 (4.6×150 mm) 3 μm, 40% of Methanol Flow rate: 3.0 g/min.

Enantiomer II (Compound 449): LCMS: m/z found 393.2 [M+H]⁺, RT=2.36 min, (Method A); ¹H NMR (400 MHz, DMSO-d₆): δ 13.23 (bs, 1H), 11.60 (bs, 1H), 8.13 (bs, 1H), 7.93 (d, 1H), 7.84 (s, 1H), 7.79 (t, 1H), 7.69 (t, 1H), 7.60 (d, 1H), 7.39 (d, 1H), 5.72 (s, 1H), 4.60 (d, 1H), 4.49 (d, 1H), 4.27 (d, 1H), 4.07 (d, 1H), 2.74 (s, 3H); Chiral analytical SFC: RT=3.72 min, Column: Chiralcel IG-3 (4.6×150 mm) 3 μm, 40% of Methanol Flow rate: 3.0 g/min.

### (S)-N-(8,9-Difluoro-6-oxo-1,4,5,6-tetrahydro-2H-pyrano[3,4-c]isoquinolin-1-yl)-N-methyl-1H-indazole-6-carboxamide (Compound 450)

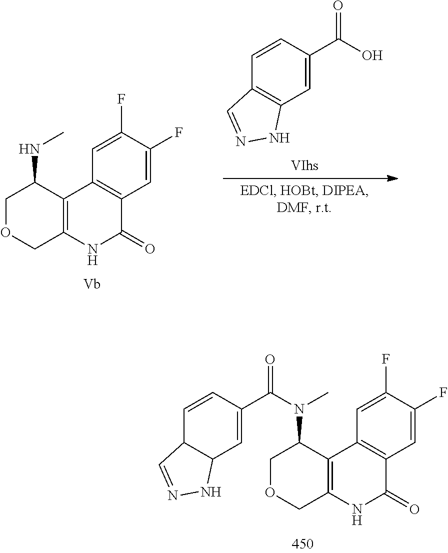 [SMILES: CN[C@@H]1COCc2[nH]c(=O)c3cc(F)c(F)cc3c21 ; O=C(O)c1ccc2cn[nH]c2c1 ; CN(C(=O)C1=CC2NN=CC2C=C1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-N-methylindolizine-6-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 1H-indazole-6-carboxylic acid (VIhs). LCMS m/z found 411.1 [M+H]⁺; RT=2.83 min (Method A); ¹H NMR (400 MHz, DMSO-d₆) δ 13.21 (bs, 1H), 11.65 (bs, 1H), 8.16 (t, 2H), 7.85 (d, 1H), 7.56 (m, 2H), 7.09 (d, 1H), 5.72 (s, 1H), 4.60 (d, 1H), 4.49 (d, 1H), 4.32 (d, 1H), 4.07 (d, 1H), 2.71 (s, 3H).

### (S)-N-(8,9-difluoro-6-oxo-1,4,5,6-tetrahydro-2H-pyrano[3,4-c]isoquinolin-1-yl)-N-methyl-1H-benzo[d]imidazole-6-carboxamide (Compound 451)

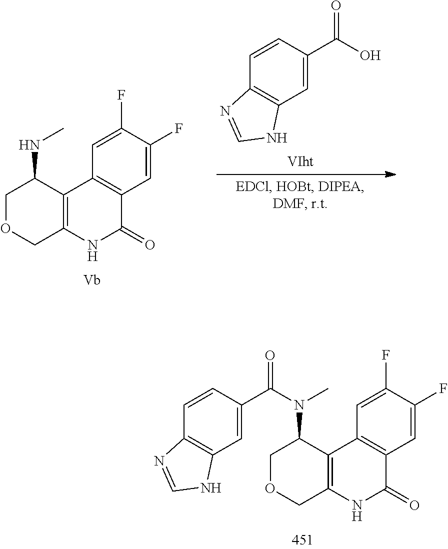 [SMILES: CN[C@@H]1COCc2[nH]c(=O)c3cc(F)c(F)cc3c21 ; O=C(O)c1ccc2nc[nH]c2c1 ; CN(C(=O)c1ccc2nc[nH]c2c1)[C@@H]1COCc2[nH]c(=O)c3cc(F)c(F)cc3c21]

Enantiomerically pure (S)-N-(8,9-difluoro-6-oxo-1,4,5,6-tetrahydro-2H-pyrano[3,4-c]isoquinolin-1-yl)-N-methyl-1H-benzo[d]imidazole-6-carboxamide was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 1H-benzo[d]imidazole-6-carboxylic acid (VIht). LCMS m/z found 411.1 [M+H]⁺; RT=1.82 min (Method A); ¹H NMR (400 MHz, DMSO-d₆) δ 12.16 (bs, 1H), 8.30 (s, 1H), 8.13 (t, 1H), 7.65-7.52 (m, 3H), 7.22 (d, 1H), 5.71 (s, 1H), 4.58 (d, 1H), 4.46 (d, 1H), 4.29 (d, 1H), 4.05 (d, 1H), 2.74 (s, 3H).

### 2-Chloro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide (Compounds 452)

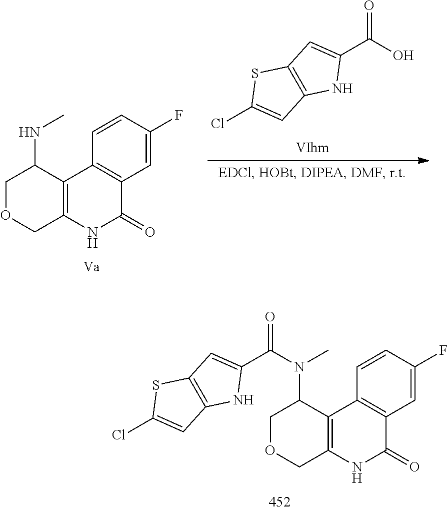

Racemic 2-chloro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide was synthesized in an analogous manner as described above from racemic 8-fluoro-1-(methylamino)-1,5-dihydro-2H-pyrano[3,4-c]isoquinolin-6(4H)-one and (Va) 2-chloro-4H-thieno[3,2-b]pyrrole-5-carboxylic acid (VIhm). LCMS: m/z found 432.0 [M+H]⁺, RT=4.33 min, (Method A); ¹H NMR (400 MHz, DMSO-d₆): δ 11.94 (bs, 1H), 11.62 (bs, 1H), 7.91-7.88 (m, 1H), 7.62 (t, 1H), 7.50 (bs, 1H), 7.09 (s, 1H), 6.89 (s, 1H), 5.71 (s, 1H), 4.62 (d, 1H), 4.47 (d, 1H), 4.12 (d, 1H), 4.02 (d, 1H), 3.09 (bs, 3H).

### 6-Fluoro-N-(8-fluoro-6-oxo-1,4,5,6-tetrahydro-2H-pyrano[3,4-c]isoquinolin-1-yl)-N,4-dimethyl-1H-indole-2-carboxamide (Compound 455)

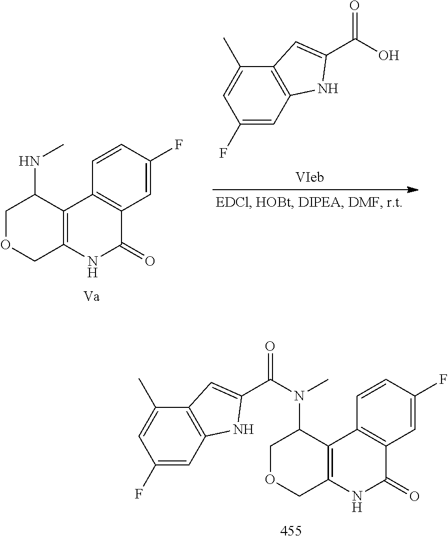

Racemic 6-fluoro-N-(8-fluoro-6-oxo-1,4,5,6-tetrahydro-2H-pyrano[3,4-c]isoquinolin-1-yl)-N,4-dimethyl-1H-indole-2-carboxamide was synthesized in an analogous manner as described above from racemic 8-fluoro-1-(methylamino)-1,5-dihydro-2H-pyrano[3,4-c]isoquinolin-6(4H)-one (Va) and 6-fluoro-4-methyl-1H-indole-2-carboxylic acid (VIeb). LCMS: m/z found 424.4 [M+H]⁺, RT=4.30 min, (Method A); ¹H NMR (400 MHz, DMSO-d₆): δ 11.72 (s, 1H), 11.63 (s, 1H), 7.92-7.89 (m, 1H), 7.65-7.53 (m, 2H), 6.98 (t, 2H), 6.73 (d, 1H), 5.76 (s, 1H), 4.64 (d, 1H), 4.48 (d, 1H), 4.16 (d, 1H), 4.05 (d, 1H), 3.17 (s, 3H), 2.46 (s, 3H).

### (S)-1-((4-bromo-3-fluorobenzyl)(methyl)amino)-8,9-difluoro-1,5-dihydro-2H-pyrano[3,4-c]isoquinolin-6(4H)-one (Compound 486)

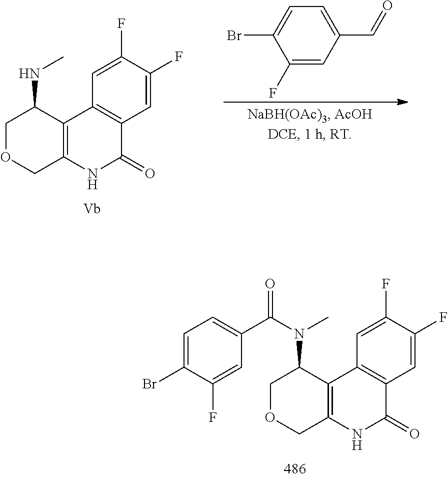 [SMILES: CN[C@@H]1COCc2[nH]c(=O)c3cc(F)c(F)cc3c21 ; O=Cc1ccc(Br)c(F)c1 ; CN(C(=O)c1ccc(Br)c(F)c1)[C@@H]1COCc2[nH]c(=O)c3cc(F)c(F)cc3c21]

To a stirred solution of enantiomerically pure (S)-8,9-difluoro-1-(methylamino)-1,5-dihydro-2H-pyrano[3,4-c]isoquinolin-6(4H)-one (Vb, 200 mg, 0.99 mmol) and 4-bromo-3-fluorobenzaldehyde (0.24 g, 1.19 mmol) in 2 mL of DCE at room temperature was added catalytic amount of acetic acid followed by sodium triacetoxyborohydride (0.53 g, 2.50 mmol). The resulting mixture was stirred at room temperature for 1 h. The reaction mixture was poured into ice cold water (10 mL), extracted with EtOAc (2×50 mL), dried over sodium sulfate, and concentrated under vacuum. The crude compound was purified by chiral SFC to afford (S)-1-((4-bromo-3-fluorobenzyl)(methyl)amino)-8,9-difluoro-1,5-dihydro-2H-pyrano[3,4-c]isoquinolin-6(4H)-one (20 mg, 17%). LCMS m/z found 453.1/455.1 [M+H]⁺; RT=3.31 min (Method A); ¹H NMR (400 MHz, DMSO-d₆) δ 11.12 (bs, 1H), 8.06 (t, 1H), 7.92 (t, 1H), 7.58 (t, 1H), 7.08 (d, 1H), 6.94 (d, 1H), 4.50 (t, 2H), 4.37 (d, 1H) 3.89 (s, 1H) 3.83 (d, 1H), 3.61 (d, 2H), 2.25 (s, 3H).

### (S)-1-(((5,6-difluoro-1H-indol-2-yl)methyl)(methyl)amino)-8,9-difluoro-1,5-dihydro-2H-pyrano[3,4-c]isoquinolin-6(4H)-one (Compound 494)

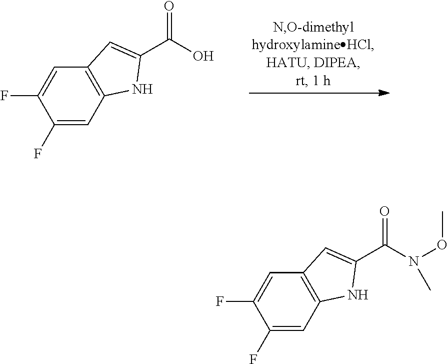 [SMILES: O=C(O)c1cc2cc(F)c(F)cc2[nH]1 ; CON(C)C(=O)c1cc2cc(F)c(F)cc2[nH]1]

Step i: To a stirred solution of 5,6-difluoro-1H-indole-2-carboxylic acid (0.30 g, 1.52 mmol) in 1 mL of THE at room temperature was added DIPEA (6.1 mL, 15.5 mmol) and HATU (0.86 g, 2.28 mmol). After stirring for 15 min at room temperature, N,O-dimethyl hydroxylamine. HCl (178 mg, 1.82 mmol) was added. After stirring at room temperature for a further 16 h, the reaction mixture was poured into ice cold saturated sodium bicarbonate solution (10 mL) and stirred for 30 minutes. A solid precipitate formed. The solid was collected by vacuum filtration, washed with water and dried under vacuum. The product was isolated by flash chromatography (silica gel, 30% ethyl acetate in petroleum ether) to afford 5,6-difluoro-N-methoxy-N-methyl-1H-indole-2-carboxamide (200 mg, 54%) as an off-white solid. LCMS: m/z found 241.2 [MH+]⁻.

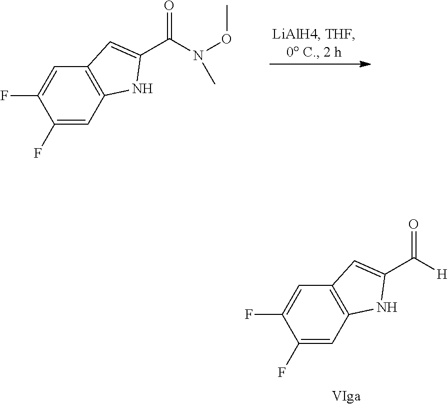 [SMILES: CON(C)C(=O)c1cc2cc(F)c(F)cc2[nH]1 ; O=Cc1cc2cc(F)c(F)cc2[nH]1]

Step ii: To a stirred solution of 180 mg (0.74 mmol, 1.0 eq.) of 5,6-difluoro-N-methoxy-N-methyl-1H-indole-2-carboxamide (180 mg, 0.74 mmol) in 2 mL of THF at 0° C. was added LiAlH₄ solution (1M in THF. 0.89 mL, 0.89 mmol). After 2 h at 0° C., the reaction mixture was poured in to ice cold saturated ammonium chloride solution (10 mL) and extracted with EtOAc (2×20 mL). The organics were dried over sodium sulfate and concentrated in vacuo to provide crude 5,6-difluoro-1H-indole-2-carbaldehyde (VIga, 130 mg). LCMS: m/z found 181.9[MH+]⁻.

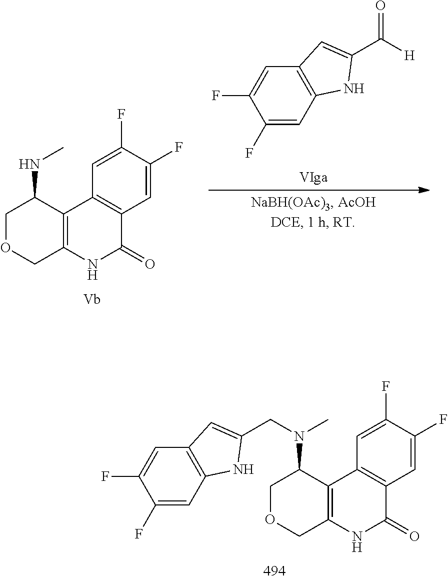 [SMILES: CN[C@@H]1COCc2[nH]c(=O)c3cc(F)c(F)cc3c21 ; O=Cc1cc2cc(F)c(F)cc2[nH]1 ; CN(Cc1cc2cc(F)c(F)cc2[nH]1)[C@@H]1COCc2[nH]c(=O)c3cc(F)c(F)cc3c21]

Step iii: Enantiomerically pure (S)-1-(((5,6-difluoro-1H-indol-2-yl)methyl)(methyl)amino)-8,9-difluoro-1,5-dihydro-2H-pyrano[3,4-c]isoquinolin-6(4H)-one was synthesized in an analogous manner as described above from enantiomerically pure (S)-8,9-difluoro-1-(methylamino)-1,5-dihydro-2H-pyrano[3,4-c]isoquinolin-6(4H)-one (Vb) and 5,6-difluoro-1H-indole-2-carbaldehyde (VIga). LCMS m/z found 432.1 [M+H]⁺; RT=3.23 min (Method A); ¹H NMR (400 MHz, DMSO-d₆) δ 11.44 (bs, 1H), 11.05 (bs, 1H), 8.0 (t, 1H), 7.75-7.70 (m, 1H), 7.40 (t, 1H), 7.28-7.24 (m, 1H), 6.22 (s, 1H), 4.52-4.47 (m, 2H), 4.34 (d, 1H), 3.92 (d, 2H), 3.79 (d, 1H), 3.59-3.55 (m, 1H), 2.32 (s, 3H).

### 8,9-Difluoro-2,3,4,5-tetrahydrophenanthridine-1,6-dione (IVn)

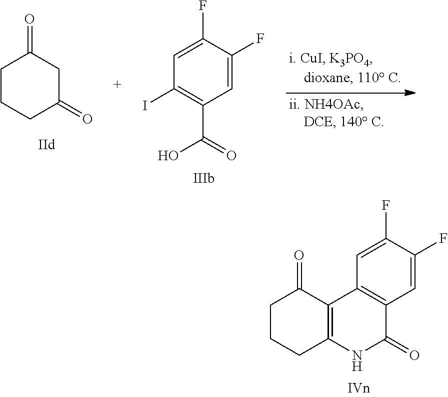 [SMILES: O=C1CCCC(=O)C1 ; O=C(O)c1cc(F)c(F)cc1I ; O=C1CCCc2[nH]c(=O)c3cc(F)c(F)cc3c21]

Step i: 8,9-Difluoro-3,4-dihydro-2H-benzo[c]chromene-1,6-dione was synthesized in an analogous manner as described above from 4,5-difluoro-2-iodo-benzoic acid (IIIb) and cyclohexane-1,3-dione (IId). ¹H NMR (400 MHz, CDCl₃) δ 8.99 (dd, 1H), 8.04 (dd, 1H), 2.94 (t, 2H), 2.73-2.58 (m, 2H), 2.24-2.12 (m, 2H).

Step ii: 8,9-Difluoro-2,3,4,5-tetrahydrophenanthridine-1,6-dione was synthesized in an analogous manner as described above from 8,9-difluoro-3,4-dihydro-2H-benzo[c]chromene-1,6-dione and ammonium acetate. LCMS: m/z found 250.1 [M+H]⁺; RT=0.90 min (Method B); ¹H NMR (400 MHz, DMSO-d₆) δ 12.15 (s, 1H), 9.14 (dd, 1H), 8.06 (dd, 1H), 2.88 (t, 2H), 2.54 (dd, 2H), 2.02 (h, 2H).

### 8,9-Difluoro-1-(methylamino)-2,3,4,5-tetrahydro-1H-phenanthridin-6-one (Vu)

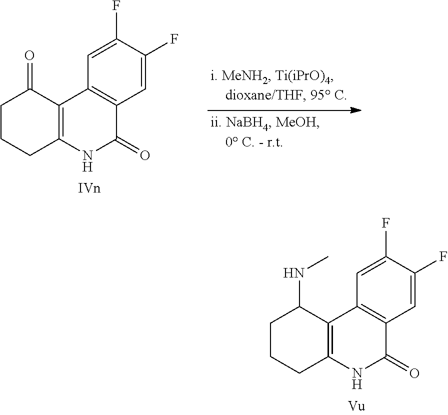 [SMILES: O=C1CCCc2[nH]c(=O)c3cc(F)c(F)cc3c21 ; CNC1CCCc2[nH]c(=O)c3cc(F)c(F)cc3c21]

Tetraisopropoxytitanium (0.48 mL, 1.58 mmol) was added to a mixture of 8,9-difluoro-2,3,4,5-tetrahydrophenanthridine-1,6-dione (0.1 g, 0.40 mmol) and a 2 M methylamine solution in THF (0.36 mL, 0.71 mmol) in 1,4-dioxane (5 mL). The mixture was stirred under nitrogen at room temperature for 2 h. An additional 0.1 mL of a 2 M solution of methylamine and 0.2 mL of tetraisopropoxytitanium were added to the reaction and stirring was continued at room temperature for 2 hours and then at 45° C. for a further 1 h. The reaction mixture was diluted with 2 mL of anhydrous methanol and cooled in an ice bath. Sodium borohydride (30 mg, 0.79 mmol) was added in one portion, the reaction mixture was stirred for 5 minutes and the ice bath was removed. After an additional 15 min the reaction was quenched by the addition of brine (1.5 mL), diluted with 20 mL of ethyl acetate, and stirred for additional 15 min. The mixture was filtered through CELITE® and the filter cake was washed with 25 mL of ethyl acetate. The product was isolated by flash-chromatography (silica gel, MeOH/DCM 0-10%) to provide 84 mg (80% yield) of 8,9-difluoro-1-(methylamino)-2,3,4,5-tetrahydro-1H-phenanthridin-6-one. LCMS: m/z found 265.2 [M+H]⁺, 234.1 [M−MeNH]⁺; RT=0.70 min, (Method B); ¹H NMR (400 MHz, CDCl₃) δ 8.02 (dd, 1H), 7.33 (dd, 1H), 3.65 (dd, 1H), 3.59-3.37 (bs, exchangeable protons), 2.59-2.45 (m, 5H), 2.21-2.11 (m, 1H), 1.96-1.79 (m, 1H), 1.78 (tt, 1H), 1.49 (tt, 1H).

### N-(8,9-Difluoro-6-oxo-1,2,3,4,5,6-hexahydrophenanthridin-1-yl)-N-methyl-1H-indole-2-carboxamide (Compounds 466 and 467)

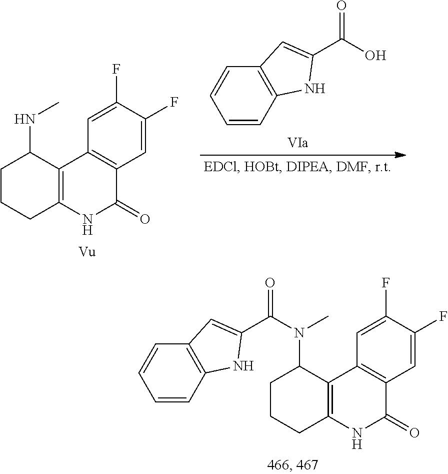

Racemic N-(8,9-difluoro-6-oxo-1,2,3,4,5,6-hexahydrophenanthridin-1-yl)-N-methyl-1H-indole-2-carboxamide was synthesized in an analogous manner as described above from racemic 8,9-difluoro-1-(methylamino)-1,3,4,5-tetrahydrophenanthridin-6(2H)-one (Vu) and 1H-indole-2-carboxylic acid (VIa). The enantiomers were subsequently separated by preparative SFC: Method isocratic, Mobile phase methanol:CO₂—40:60. Column: ChiralPak IC-3 (30×250 mm), 5 μm, flow rate: 110 g/min.

Enantiomer I (Compound 466): LCMS: m/z found 408.1 [M+H]⁺, RT=4.24 min, (Method A); ¹H NMR (400 MHz, DMSO-d₆): δ 11.70 (bs, 1H), 11.5 (bs, 1H), 8.11 (t, 1H), 7.60 (d, 1H), 7.47 (d, 1H), 7.27-7.19 (m, 2H), 7.05 (t, 1H), 6.91 (s, 1H) 5.92 (s, 1H), 2.9 (s, 3H), 2.74 (t, 1H), 2.6 (t, 1H), 2.01 (t, 1H), 1.9 (d, 2H), 1.79 (m, 1H); Chiral analytical SFC: RT=5.28 min, Column: Chiralpak IC-3 (4.6×150 mm) 3 μm, 30% of Methanol with 0.5% DEA Flow rate: 3.0 g/min.

Enantiomer II (Compound 467): LCMS: m/z found 408.1 [M+H]⁺, RT=4.24 min, (Method A); ¹H NMR (400 MHz, DMSO-d₆): δ 11.70 (bs, 1H), 11.5 (bs, 1H), 8.11 (t, 1H), 7.60 (d, 1H), 7.47 (d, 1H), 7.27-7.19 (m, 2H), 7.05 (t, 1H), 6.91 (s, 1H) 5.92 (s, 1H), 2.9 (s, 3H), 2.74 (t, 1H), 2.6 (t, 1H), 2.01 (t, 1H), 1.9 (d, 2H), 1.79 (m, 1H); Chiral analytical SFC: RT=8.20 min, Column: Chiralpak IC-3 (4.6×150 mm) 3 μm, 30% of Methanol with 0.5% DEA Flow rate: 3.0 g/min.

### 8-Fluoro-2,3,4,5-tetrahydrophenanthridine-1,6-dione (IVo)

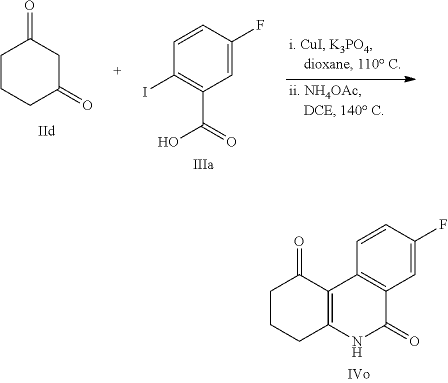 [SMILES: O=C1CCCC(=O)C1 ; O=C(O)c1cc(F)ccc1I ; O=C1CCCc2[nH]c(=O)c3cc(F)ccc3c21]

Step i: 5-Fluoro-2-iodo-benzoic acid (IIIa, 543.7 mg, 2.04 mmol), cyclohexane-1,3-dione (IId, 275.02 mg, 2.45 mmol), copper (I) iodide (38.93 mg, 0.20 mmol), and potassium phosphate (606.64 mg, 2.86 mmol) were combined in a tube under a nitrogen atmosphere. Anhydrous 1,4-dioxane (1.5 mL) was added and the reaction tube was purged with nitrogen and stirred at room temperature for 30 min, and then heated at 110° C. for 4 h. The reaction mixture was allowed to cool to room temperature, diluted with ethyl acetate (10 mL), filtered through CELITE®, and the pad was washed with ethyl acetate (3×25 mL). The combined organic extracts were dried over sodium sulfate, filtered and the solvent was evaporated under high vacuum to afford 434 mg (91% yield) of 8-fluoro-3,4-dihydro-2H-benzo[c]chromene-1,6-dione of satisfactory purity. ¹H NMR (400 MHz, CDCl₃) δ 9.11 (ddd, 1H), 7.91 (ddd, 1H), 7.49 (ddd, 1H), 2.93 (t, 2H), 2.69-2.58 (m, 2H), 2.23-2.11 (m, 2H).

Step ii: 8-fluoro-3,4-dihydro-2H-benzo[c]chromene-1,6-dione (434.00 mg, 1.87 mmol) from Step i and ammonium acetate (1.44 g, 18.69 mmol) were stirred in 1,2-dichloroethane (2 mL) at 140° C. in sealed tube for 10 h. The reaction mixture was cooled and diluted with dichloromethane and washed with saturated ammonium chloride. The aqueous phase was extracted three times with dichloromethane and the combined organic extracts were dried over sodium sulfate, then filtered. The solvent was evaporated, and the product was isolated by flash chromatography (silicagel, methanol/dichloromethane 0-5% gradient), to provide 225 mg (52% yield) of 8-fluoro-2,3,4,5-tetrahydrophenanthridine-1,6-dione (IVo). LCMS: m/z found 232.1 [M+H]⁺; RT=0.82 min, (Method B); ¹H NMR (400 MHz, CDCl₃) δ 9.21 (dd, 1H), 7.87 (ddd, 1H), 7.38 (dddd, 1H), 2.83 (t, 2H), 2.57 (dd, 2H), 2.09 (dt, 2H).

### 8-Fluoro-1-(methylamino)-2,3,4,5-tetrahydro-1H-phenanthridin-6-one (Vv)

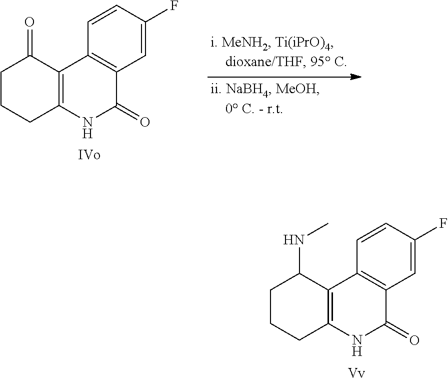 [SMILES: O=C1CCCc2[nH]c(=O)c3cc(F)ccc3c21 ; CNC1CCCc2[nH]c(=O)c3cc(F)ccc3c21]

8-Fluoro-1-(methylamino)-2,3,4,5-tetrahydro-1H-phenanthridin-6-one was synthesized in an analogous manner as described above from 8-fluoro-2,3,4,5-tetrahydrophenanthridine-1,6-dione (IVo) and methanamine. LCMS: m/z found 216.1 [M−MeNH)]⁺; RT=0.60 min, (Method B); ¹H NMR (400 MHz, CDCl₃) δ 10.49 (s, 1H), 8.12-7.97 (m, 1H), 7.69 (dd, 1H), 7.43 (ddd, 1H), 3.86-3.82 (m, 1H), 2.65 (dd, 1H), 2.58 (s, 3H), 2.24 (dd, 1H), 2.08-1.91 (m, 1H), 1.85 (dt, 1H), 1.56 (tt, 1H), 1.25 (m, 1H).

### N-(8-Fluoro-6-oxo-1,2,3,4,5,6-hexahydrophenanthridin-1-yl)-N-methyl-1H-indole-2-carboxamide (Compounds 475 and 476)

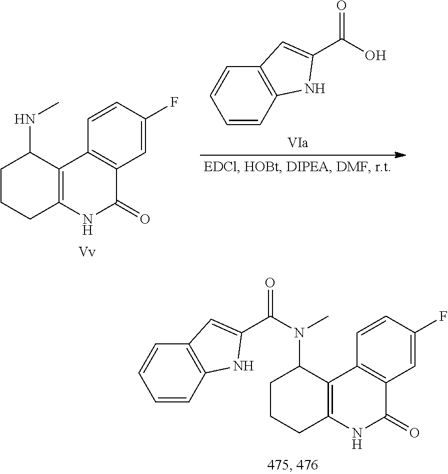

Racemic N-(8-fluoro-6-oxo-1,2,3,4,5,6-hexahydrophenanthridin-1-yl)-N-methyl-1H-indole-2-carboxamide was synthesized in an analogous manner as described above from racemic 8-fluoro-1-(methylamino)-1,3,4,5-tetrahydrophenanthridin-6(2H)-one (Vv) and 1H-indole-2-carboxylic acid (VIa). The enantiomers were subsequently separated by preparative SFC: Method isocratic, Mobile phase methanol:CO₂—40:60. Column: ChiralPak IC-3 (30×250 mm), 5 μm, flow rate: 110 g/min.

Enantiomer I (Compound 475): LCMS: m/z found 390.1 [M+H]⁺, RT=4.06 min, (Method A); ¹H NMR (400 MHz, DMSO-d₆): δ 11.70 (bs, 1H), 11.5 (bs, 1H), 7.87-7.82 (dd, 1H), 7.59-7.58 (t, 2H), 7.45 (m, 2H), 7.2 (t, 1H), 7.05 (t, 1H), 6.9 (s, 1H), 5.9 (s, 1H), 2.9 (s, 3H), 2.68 (t, 1H), 2.62 (m, 1H), 2.06 (m, 1H), 1.94-1.91 (m, 3H); Chiral analytical SFC: RT=4.81 min, Column: Chiralpak IC-3 (4.6×150 mm) 3 μm, 50% of Methanol/Acetonitrile (1:1) Flow rate: 3.0 g/min.

Enantiomer II (Compound 476): LCMS: m/z found 390.1 [M+H]⁺, RT=4.06 min, (Method A); ¹H NMR (400 MHz, DMSO-d₆): δ 11.70 (bs, 1H), 11.5 (bs, 1H), 7.87-7.82 (dd, 1H), 7.59-7.58 (t, 2H), 7.45 (m, 2H), 7.2 (t, 1H), 7.05 (t, 1H), 6.9 (s, 1H), 5.9 (s, 1H), 2.9 (s, 3H), 2.68 (t, 1H), 2.62 (m, 1H), 2.06 (m, 1H), 1.94-1.91 (m, 3H); Chiral analytical SFC: RT=6.38 min, Column: Chiralpak IC-3 (4.6×150 mm) 3 μm, 50% of Methanol/Acetonitrile (1:1) Flow rate: 3.0 g/min.

### (R)-N-(8,9-difluoro-6-oxo-1,2,3,4,5,6-hexahydrophenanthridin-1-yl)-5-fluoro-N-methyl-1H-indole-2-carboxamide (Compound 480)

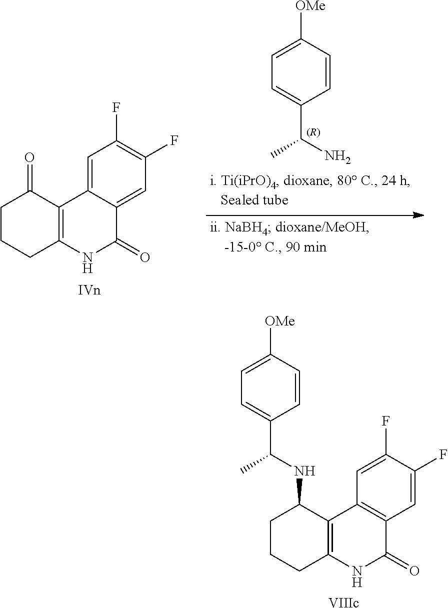

Step i: To a stirred solution of 8,9-difluoro-2H-pyrano[3,4-c]isoquinoline-1,6(4H,5H)-dione (IVn, 3.0 g, 11.9 mmol) in 30 mL of 1,4-dioxane at room temperature under inert atmosphere were added of (R)-1-(4-methoxyphenyl)ethan-1-amine (2.1 mL, 14.31 mmol) followed by 15 mL (5 vol) of titanium isopropoxide. The reaction mixture was heated to 90° C. for 24 h. The reaction was cooled to 0° C. and diluted with methanol (2 mL). To this mixture, NaBH₄ (85 mg, 2.20 mmol) was added portion-wise at 0° C. and stirred at room temperature for 4 h. After completion of reaction, the reaction mixture was diluted with water (50 mL). The heterogeneous mixture was filtered and washed with ethyl acetate (50 mL). The organic layer was separated, and the aqueous layer was extracted with ethyl acetate (3×100 mL). The combined organics were dried over anhydrous Na₂SO₄ and concentrated under reduced pressure. The resulting crude product was triturated with diethyl ether (10 mL). The resulting precipitate was collected and dried under vacuum to afford 8,9-difluoro-1-(((R)-1-(4-methoxyphenyl)ethyl)amino)-1,3,4,5-tetrahydrophenanthridin-6(2H)-one (VIIIc, 1.7 g, 36%). LCMS: m/z found 385.22 [M+H]⁺.

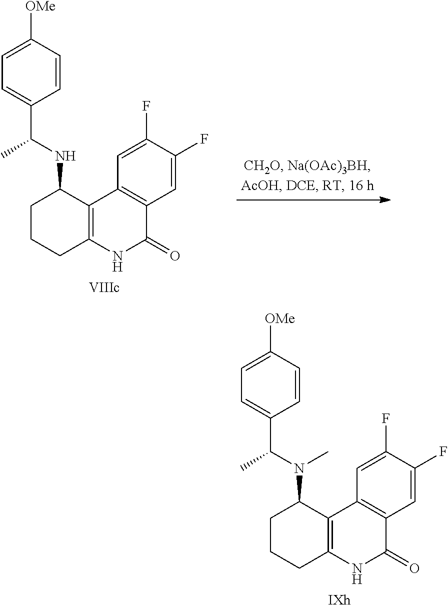 [SMILES: COc1ccc([C@@H](C)N[C@@H]2CCCc3[nH]c(=O)c4cc(F)c(F)cc4c32)cc1 ; COc1ccc([C@@H](C)N(C)[C@@H]2CCCc3[nH]c(=O)c4cc(F)c(F)cc4c32)cc1]

Step ii: To a stirred solution of 8,9-difluoro-1-(((R)-1-(4-methoxyphenyl)ethyl)amino)-1,3,4,5-tetrahydrophenanthridin-6(2H)-one (1.00 g, 2.5 mmol) in 10 mL of 1,2-DCE at room temperature under inert atmosphere was added formaldehyde solution (37% aqueous, 1.06 mL, 12.95 mmol) followed by three drops of acetic acid. Sodium triacetoxyborohydride (1.15 g, 3.8 mmol) was then added. After stirring at room temperature for 16 h, the reaction mixture was diluted with water (50 mL). The heterogeneous mixture was filtered and washed with ethyl acetate (50 mL). The organic layer was separated, and the aqueous layer was extracted with ethyl acetate (3×100 mL). The combined organic layers were dried over anhydrous Na₂SO₄ and concentrated under reduced pressure. The resulting crude product was triturated with diethyl ether (10 mL) and EtOH (5 mL). The precipitate was collected by filtration and dried under vacuum to afford 8,9-difluoro-1-(((R)-1-(4-methoxyphenyl)ethyl)(methyl)amino)-1,3,4,5-tetrahydrophenanthridin-6(2H)-one (IXh, 0.75 g, 72%).

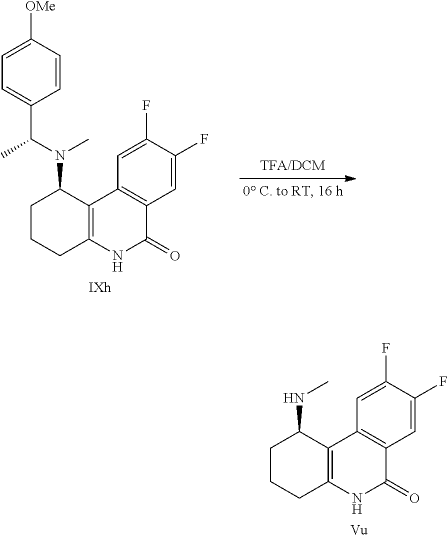 [SMILES: COc1ccc([C@@H](C)N(C)[C@@H]2CCCc3[nH]c(=O)c4cc(F)c(F)cc4c32)cc1 ; CN[C@@H]1CCCc2[nH]c(=O)c3cc(F)c(F)cc3c21]

Step iii: To a stirred solution of 8,9-difluoro-1-(((R)-1-(4-methoxyphenyl)ethyl)(methyl)amino)-1,3,4,5-tetrahydrophenanthridin-6(2H)-one (1.50 g, 0.9 mmol) in 15 mL of DCM at room temperature under inert atmosphere was added 7.5 mL of TFA. After stirring at room temperature for 24, the reaction mixture was concentrated under reduced pressure, and then basified with 10% saturated sodium carbonate solution (50 mL). The organic layer was separated, and the aqueous layer was extracted with ethyl acetate (3×100 mL). The combined organic layers were dried over anhydrous Na₂SO₄ and concentrated under reduced pressure. The resulting crude product was triturated with diethyl ether (20 mL). The resulting solid was filtered and dried under vacuum to afford (R)-8,9-difluoro-1-(methylamino)-1,3,4,5-tetrahydrophenanthridin-6(2H)-one (Vu, 600 mg, 60%). LCMS m/z 263.25 found [M−H]⁻. H NMR (400 MHz, DMSO-d₆): 11.26 (bs, 1H), 8.01 (m, 1H), 7.63 (m, 1H), 3.66 (s, 1H), 2.47 (t, 2H), 2.39 (s, 3H), 2.13 (d, 1H), 1.87 (m, 1H), 1.66 (d, 2H), 1.43 (t, 1H).

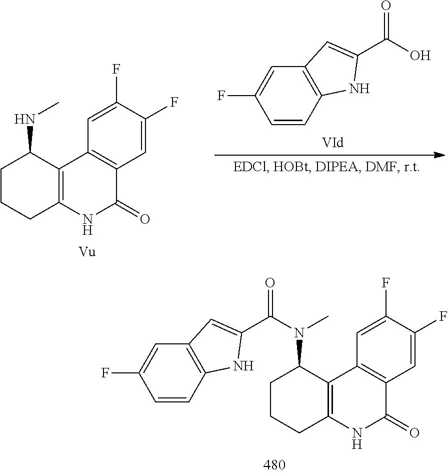 [SMILES: CN[C@@H]1CCCc2[nH]c(=O)c3cc(F)c(F)cc3c21 ; O=C(O)c1cc2cc(F)ccc2[nH]1 ; CN(C(=O)c1cc2cc(F)ccc2[nH]1)[C@@H]1CCCc2[nH]c(=O)c3cc(F)c(F)cc3c21]

Step iv: Enantiomerically pure (R)-N-(8,9-difluoro-6-oxo-1,2,3,4,5,6-hexahydrophenanthridin-1-yl)-5-fluoro-N-methyl-1H-indole-2-carboxamide was synthesized in an analogous manner as described above from enantiomerically pure (R)-8,9-difluoro-1-(methylamino)-1,3,4,5-tetrahydrophenanthridin-6(2H)-one (Vu) and 5-fluoro-1H-indole-2-carboxylic acid (VId). LCMS m/z found 426.2 [M+H]⁺; RT=4.38 min (Method A); ¹H NMR (400 MHz, DMSO-d₆) δ 11.9 (bs, 1H), 11.59 (bs, 1H), 8.09 (t, 1H), 7.50-7.44 (m, 1H), 7.35 (d, 1H), 7.27-7.22 (m, 1H), 7.08 (t, 1H), 6.89 (bs, 1H), 5.91 (s, 1H), 2.96 (s, 3H), 2.75-2.59 (m, 2H), 2.07-1.79 (m, 4H).

### (R)-N-(8,9-difluoro-6-oxo-1,2,3,4,5,6-hexahydrophenanthridin-1-yl)-5,6-difluoro-N-methyl-1H-indole-2-carboxamide (Compound 481)

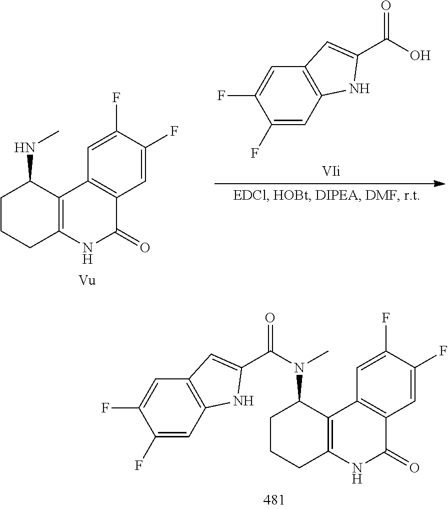 [SMILES: CN[C@@H]1CCCc2[nH]c(=O)c3cc(F)c(F)cc3c21 ; O=C(O)c1cc2cc(F)c(F)cc2[nH]1 ; CN(C(=O)c1cc2cc(F)c(F)cc2[nH]1)[C@@H]1CCCc2[nH]c(=O)c3cc(F)c(F)cc3c21]

Enantiomerically pure (R)-N-(8,9-difluoro-6-oxo-1,2,3,4,5,6-hexahydrophenanthridin-1-yl)-5,6-difluoro-N-methyl-1H-indole-2-carboxamide was synthesized in an analogous manner as described above from enantiomerically pure (R)-8,9-difluoro-1-(methylamino)-1,3,4,5-tetrahydrophenanthridin-6(2H)-one (Vu) and 5,6-difluoro-1H-indole-2-carboxylic acid (VIi). LCMS m/z found 444.2 [M+H]⁺; RT=4.53 min (Method A); ¹H NMR (400 MHz, DMSO-d₆) δ 11.89 (bs, 1H), 11.6 (bs, 1H), 8.09 (t, 1H), 7.61 (t, 1H), 7.38 (t, 1H), 7.24 (t, 1H), 6.93 (bs, 1H), 5.91 (s, 1H), 2.96 (s, 3H), 2.75-2.58 (m, 2H), 2.07-1.79 (m, 4H).

### (R)-N-(8,9-difluoro-6-oxo-1,2,3,4,5,6-hexahydrophenanthridin-1-yl)-6-(difluoromethyl)-5-fluoro-N-methyl-1H-indole-2-carboxamide (Compound 482)

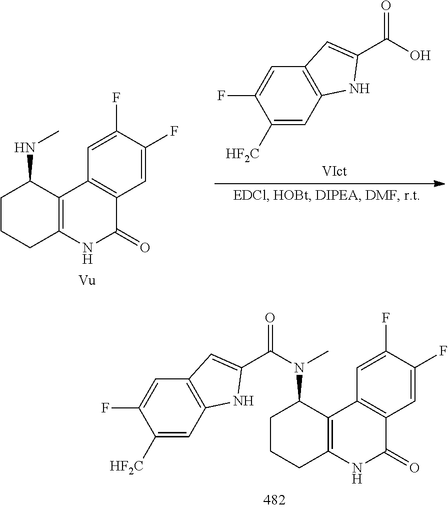

Enantiomerically pure (R)-N-(8,9-difluoro-6-oxo-1,2,3,4,5,6-hexahydrophenanthridin-1-yl)-6-(difluoromethyl)-5-fluoro-N-methyl-1H-indole-2-carboxamide was synthesized in an analogous manner as described above from enantiomerically pure (R)-8,9-difluoro-1-(methylamino)-1,3,4,5-tetrahydrophenanthridin-6(2H)-one (Vu) and 6-(difluoromethyl)-5-fluoro-1H-indole-2-carboxylic acid (VIct). LCMS m/z found 476.1 [M+H]⁺; RT=4.59 min (Method A); ¹H NMR (400 MHz, DMSO-d₆) δ 12.19 (bs, 1H), 11.6 (bs, 1H), 8.09 (t, 1H), 7.70 (d, 1H), 7.53 (d, 1H), 7.39-7.11 (m, 2H), 6.96 (bs, 1H), 5.91 (s, 1H), 2.96 (s, 3H), 2.76-2.54 (m, 2H), 2.07-1.79 (m, 4H).

### (R)-N-(8,9-difluoro-6-oxo-1,2,3,4,5,6-hexahydrophenanthridin-1-yl)-4-(difluoromethyl)-6-fluoro-N-methyl-1H-indole-2-carboxamide (Compound 483)

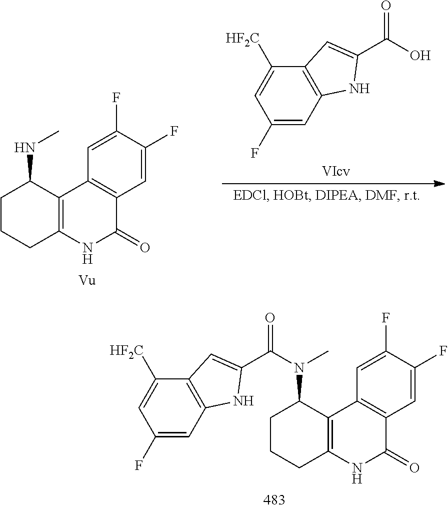

Enantiomerically pure (R)-N-(8,9-difluoro-6-oxo-1,2,3,4,5,6-hexahydrophenanthridin-1-yl)-4-(difluoromethyl)-6-fluoro-N-methyl-1H-indole-2-carboxamide was synthesized in an analogous manner as described above from enantiomerically pure (R)-8,9-difluoro-1-(methylamino)-1,3,4,5-tetrahydrophenanthridin-6(2H)-one (Vu) and 4-(difluoromethyl)-6-fluoro-1H-indole-2-carboxylic acid (VIcv). LCMS m/z found 476.1 [M+H]⁺; RT=4.58 min (Method A); ¹H NMR (400 MHz, DMSO-d₆) δ 12.16 (bs, 1H), 11.6 (bs, 1H), 8.09 (t, 1H), 7.77-7.17 (m, 4H), 7.04 (bs, 1H), 5.91 (s, 1H), 2.97 (s, 3H), 2.76-2.59 (m, 2H), 2.07-1.79 (m, 4H).

### (R)-N-(8,9-difluoro-6-oxo-1,2,3,4,5,6-hexahydrophenanthridin-1-yl)-N,4-dimethyl-1H-indole-2-carboxamide (Compound 484)

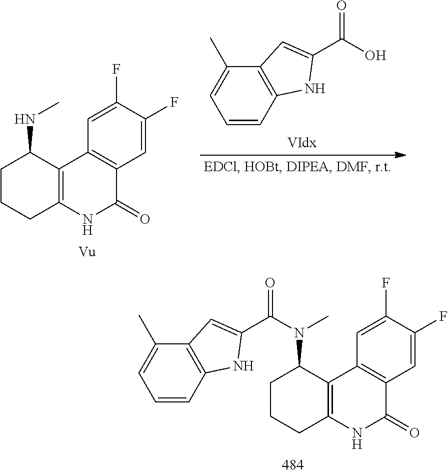

Enantiomerically pure (R)-N-(8,9-difluoro-6-oxo-1,2,3,4,5,6-hexahydrophenanthridin-1-yl)-N,4-dimethyl-1H-indole-2-carboxamide was synthesized in an analogous manner as described above from enantiomerically pure (R)-8,9-difluoro-1-(methylamino)-1,3,4,5-tetrahydrophenanthridin-6(2H)-one (Vu) and 4-methyl-1H-indole-2-carboxylic acid (VIdx). LCMS m/z found 422.2 [M+H]⁺; RT=4.55 min (Method A); ¹H NMR (400 MHz, DMSO-d₆) δ 11.67 (bs, 1H), 11.59 (bs, 1H), 8.09 (t, 1H), 7.29 (d, 2H), 7.1 (t, 1H), 6.92 (bs, 1H), 6.83 (d, 1H), 5.93 (s, 1H), 3.0 (s, 3H), 2.82-2.56 (m, 2H), 2.46 (s, 3H), 2.08-1.79 (m, 4H).

### (R)-N-(8,9-difluoro-6-oxo-1,2,3,4,5,6-hexahydrophenanthridin-1-yl)-6-fluoro-N,4-dimethyl-1H-indole-2-carboxamide (Compound 485)

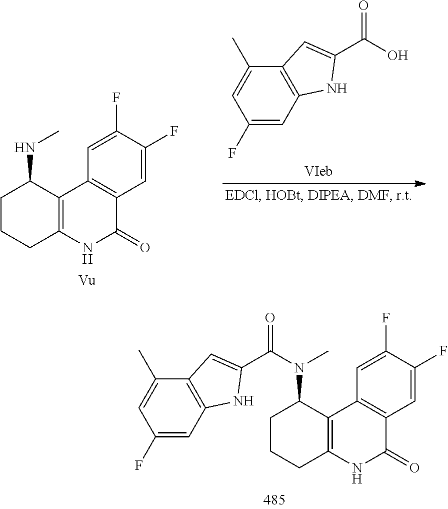 [SMILES: CN[C@@H]1CCCc2[nH]c(=O)c3cc(F)c(F)cc3c21 ; Cc1cc(F)cc2[nH]c(C(=O)O)cc12 ; Cc1cc(F)cc2[nH]c(C(=O)N(C)[C@@H]3CCCc4[nH]c(=O)c5cc(F)c(F)cc5c43)cc12]

Enantiomerically pure (R)-N-(8,9-difluoro-6-oxo-1,2,3,4,5,6-hexahydrophenanthridin-1-yl)-6-fluoro-N,4-dimethyl-1H-indole-2-carboxamide was synthesized in an analogous manner as described above from enantiomerically pure (R)-8,9-difluoro-1-(methylamino)-1,3,4,5-tetrahydrophenanthridin-6(2H)-one (Vu) and 6-fluoro-4-methyl-1H-indole-2-carboxylic acid (VIeb). LCMS m/z found 440.1 [M+H]⁺; RT=4.70 min (Method A); ¹H NMR (400 MHz, DMSO-d₆) δ 11.74 (bs, 1H), 11.6 (bs, 1H), 8.09 (t, 1H), 7.29-7.24 (m, 1H), 7.0-6.96 (m, 2H), 6.74 (d, 1H), 5.91 (s, 1H), 2.99 (s, 3H), 2.76-2.56 (m, 2H), 2.47 (s, 3H), 2.06 (bs, 1H), 1.90-1.79 (m, 3H).

### (R)-5-Fluoro-N-(8-fluoro-6-oxo-1,2,3,4,5,6-hexahydrophenanthridin-1-yl)-N-methyl-11H-indole-2-carboxamide (Compound 489)

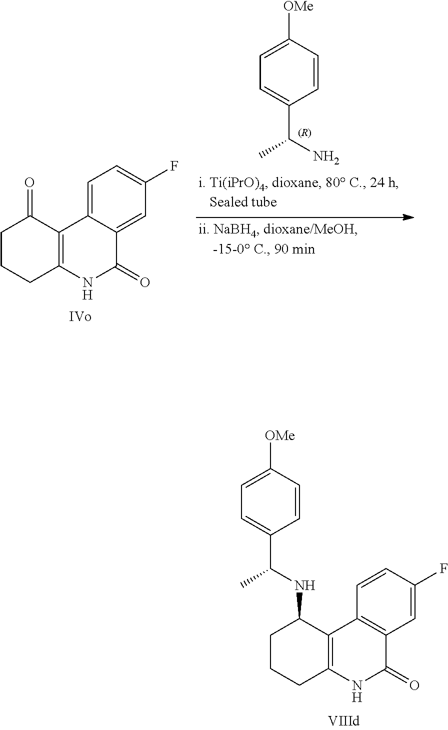

Step i: To a stirred solution of 8-fluoro-2H-pyrano[3,4-c]isoquinoline-1,6(4H,5H)-dione (IVo, 3.0 g, 11.9 mmol) in 30 mL of 1,4-dioxane at room temperature under inert atmosphere were added of (R)-1-(4-methoxyphenyl)ethan-1-amine (2.1 mL, 14.31 mmol) followed by 15 mL (5 vol) of titanium isopropoxide. The reaction mixture was heated to 90° C. for 24 h. The reaction was cooled to 0° C. and diluted with methanol (2 mL). To this mixture, NaBH₄ (85 mg, 2.20 mmol) was added portion-wise at 0° C. and stirred at room temperature for 4 h. After completion of reaction, the reaction mixture was diluted with water (50 mL). The heterogeneous mixture was filtered and washed with ethyl acetate (50 mL). The organic layer was separated, and the aqueous layer was extracted with ethyl acetate (3×100 mL). The combined organics were dried over anhydrous Na₂SO₄ and concentrated under reduced pressure. The resulting residue was triturated with diethyl ether (10 mL). The resulting precipitate was collected and dried under vacuum to afford 8-fluoro-1-(((R)-1-(4-methoxyphenyl)ethyl)amino)-1,3,4,5-tetrahydrophenanthridin-6(2H)-one (VIId, 1.7 g, 36%). LCMS: m/z found 367.15 [M+H]⁺.

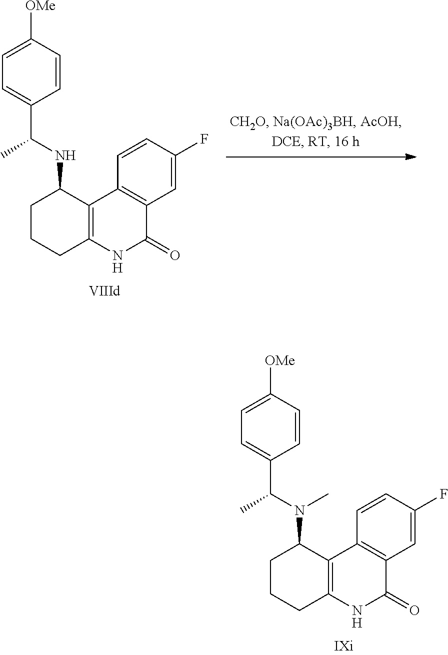 [SMILES: COc1ccc([C@@H](C)N[C@@H]2CCCc3[nH]c(=O)c4cc(F)ccc4c32)cc1 ; COc1ccc([C@@H](C)N(C)[C@@H]2CCCc3[nH]c(=O)c4cc(F)ccc4c32)cc1]

Step ii: To a stirred solution of 8-fluoro-1-(((R)-1-(4-methoxyphenyl)ethyl)amino)-1,3,4,5-tetrahydrophenanthridin-6(2H)-one (1.00 g, 2.5 mmol) in 10 mL of 1,2 DCE at room temperature under inert atmosphere were added 1.06 mL (12.95 mmol, 5.0 eq.) of 37% of formaldehyde solution (37% aqueous, 1.06 mL, 12.95 mmol) and three drops of AcOH. Sodium triacetoxyborohydride (1.15 g, 3.8 mmol) was added. After stirring at room temperature for 16 h, the reaction mixture was diluted with water (50 mL). The heterogeneous mixture was filtered and washed with ethyl acetate (50 mL). The organic layer was separated and the aqueous layer was extracted with ethyl acetate (3×100 mL). The combined organic layers were dried over anhydrous Na₂SO₄ and concentrated under reduced pressure. The resulting crude product was triturated with diethyl ether (10 mL) and EtOH (5 mL). The precipitate was collected by filtration and dried under vacuum to afford 8-fluoro-1-(((R)-1-(4-methoxyphenyl)ethyl)(methyl)amino)-1,3,4,5-tetrahydrophenanthridin-6(2H)-one (IXi, 0.75 g, 72%). LCMS: m/z found 379.1 [M−H]⁻.

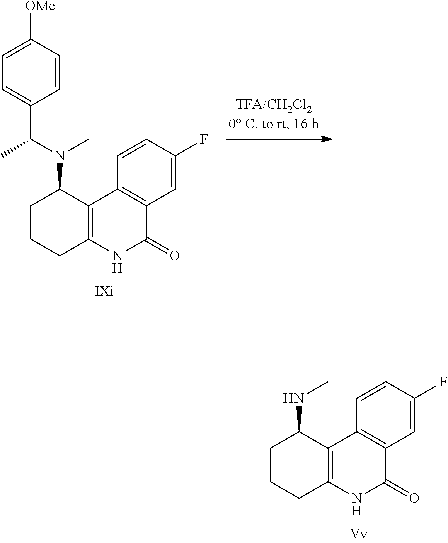 [SMILES: COc1ccc([C@@H](C)N(C)[C@@H]2CCCc3[nH]c(=O)c4cc(F)ccc4c32)cc1 ; CN[C@@H]1CCCc2[nH]c(=O)c3cc(F)ccc3c21]

Step iii: To a stirred solution 8-fluoro-1-(((R)-1-(4-methoxyphenyl)ethyl)(methyl)amino)-1,3,4,5-tetrahydrophenanthridin-6(2H)-one (1.50 g, 0.9 mmol) in 15 mL of DCM at room temperature under inert atmosphere was added 7.5 mL of TFA. After stirring at room temperature for 24, the reaction mixture was concentrated under reduced pressure, and then basified with 10% saturated sodium carbonate solution (50 mL). The organic layer was separated, and the aqueous layer was extracted with ethyl acetate (3×100 mL). The combined organic layers were dried over anhydrous Na₂SO₄ and concentrated under reduced pressure. The resulting crude product was triturated with diethyl ether (20 mL). The resulting solid was filtered and dried under vacuum to afford (R)-8-fluoro-1-(methylamino)-1,3,4,5-tetrahydrophenanthridin-6(2H)-one (Vv, 600 mg, 60%). LCMS: m/z 245.41 found [M−H]⁻.

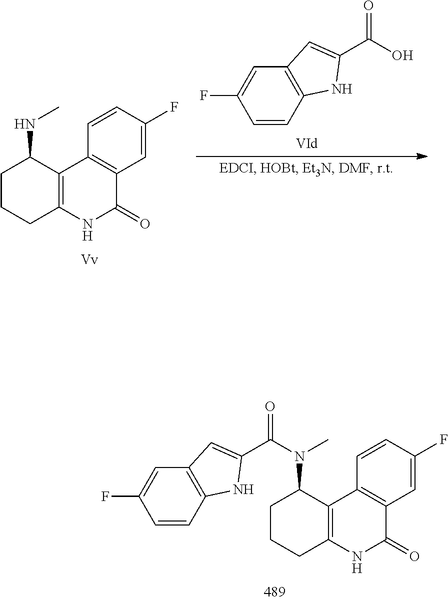

Step iv: Enantiomerically pure (R)-5-fluoro-N-(8-fluoro-6-oxo-1,2,3,4,5,6-hexahydrophenanthridin-1-yl)-N-methyl-1H-indole-2-carboxamide was synthesized in an analogous manner as described above from enantiomerically pure (R)-8-fluoro-1-(methylamino)-1,3,4,5-tetrahydrophenanthridin-6(2H)-one (Vv) and 5-fluoro-1H-indole-2-carboxylic acid (VId). LCMS m/z found 408.1 [M+H]⁺; RT=4.17 min (Method A); ¹H NMR (400 MHz, DMSO-d₆) δ 11.79 (bs, 1H), 11.51 (bs, 1H), 7.88 (d, 1H), 7.59 (t, 1H), 7.47-7.42 (m, 2H) 7.34 (d, 1H), 7.07 (t, 1H), 6.85 (s, 1H), 5.92 (s, 1H), 2.94 (s, 3H), 2.73 (m, 1H), 2.58 (m, 1H), 2.07-1.80 (m, 4H).

### (R)-6-(Difluoromethyl)-5-fluoro-N-(8-fluoro-6-oxo-1,2,3,4,5,6-hexahydrophenanthridin-1-yl)-N-methyl-1H-indole-2-carboxamide (Compound 479)

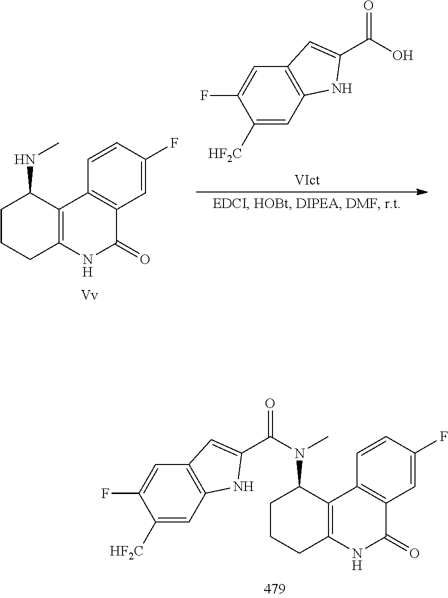

Enantiomerically pure (R)-6-(difluoromethyl)-5-fluoro-N-(8-fluoro-6-oxo-1,2,3,4,5,6-hexahydrophenanthridin-1-yl)-N-methyl-1H-indole-2-carboxamide was synthesized in an analogous manner as described above from enantiomerically pure (R)-8-fluoro-1-(methylamino)-1,3,4,5-tetrahydrophenanthridin-6(2H)-one (Vv) and 5-fluoro-6-(difluoromethyl)-1H-indole-2-carboxylic acid (VIct). LCMS m/z found 458.2 [M+H]⁺; RT=4.45 min (Method A); ¹H NMR (400 MHz, DMSO-d₆) δ 12.16 (bs, 1H), 11.5 (bs, 1H), 7.87-7.84 (t, 1H), 7.75-7.65 (d, 1H), 7.64-7.60 (t, 2H), 7.42 (m, 1H), 7.22 (t, 1H), 6.9 (s, 1H), 5.92 (s, 1H), 2.9 (s, 3H), 2.70 (m, 2H), 2.07-1.80 (m, 4H).

### (R)-5,6-Difluoro-N-(8-fluoro-6-oxo-1,2,3,4,5,6-hexahydrophenanthridin-1-yl)-N-methyl-1H-indole-2-carboxamide (Compound 490)

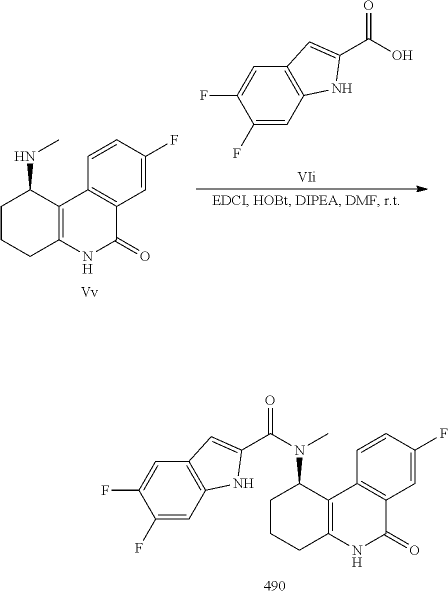 [SMILES: CN[C@@H]1CCCc2[nH]c(=O)c3cc(F)ccc3c21 ; O=C(O)c1cc2cc(F)c(F)cc2[nH]1 ; CN(C(=O)c1cc2cc(F)c(F)cc2[nH]1)[C@@H]1CCCc2[nH]c(=O)c3cc(F)ccc3c21]

Enantiomerically pure (R)-5,6-difluoro-N-(8-fluoro-6-oxo-1,2,3,4,5,6-hexahydrophenanthridin-1-yl)-N-methyl-1H-indole-2-carboxamide de was synthesized in an analogous manner as described above from enantiomerically pure (R)-8-fluoro-1-(methylamino)-1,3,4,5-tetrahydrophenanthridin-6(2H)-one (Vv) and 5,6-difluoro-1H-indole-2-carboxylic acid (VIi). LCMS m/z found 426.1 [M+H]⁺; RT=4.36 min (Method A); ¹H NMR (400 MHz, DMSO-d₆) δ 11.87 (bs, 1H), 11.51 (bs, 1H), 7.88 (dd, 1H), 7.62-7.57 (m, 2H), 7.42-7.35 (m, 2H) 6.90 (s, 1H) 5.91 (s, 1H) 2.94 (s, 3H) 2.68 (m, 1H), 2.56 (m, 1H), 2.05-1.80 (m, 4H).

### (R)-N-(8-fluoro-6-oxo-1,2,3,4,5,6-hexahydrophenanthridin-1-yl)-N,4-dimethyl-1H-indole-2-carboxamide (Compound 491)

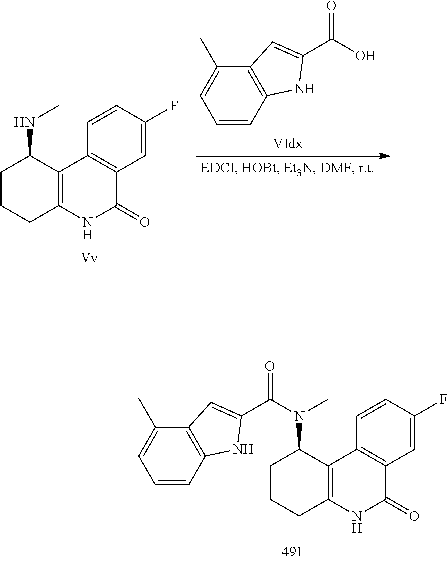

Enantiomerically (R)-N-(8-fluoro-6-oxo-1,2,3,4,5,6-hexahydrophenanthridin-1-yl)-N,4-dimethyl-1H-indole-2-carboxamide was synthesized in an analogous manner as described above from enantiomerically pure (R)-8-fluoro-1-(methylamino)-1,3,4,5-tetrahydrophenanthridin-6(2H)-one (Vv) and 4-methyl-1H-indole-2-carboxylic acid (VIdx). LCMS m/z found 404.1 [M+H]⁺; RT=4.39 min (Method A); ¹H NMR (400 MHz, DMSO-d₆) δ 11.64 (s, 1H), 11.51 (bs, 1H), 7.88 (m, 1H), 7.59 (m, 1H), 7.45 (m, 1H), 7.29 (d, 1H), 7.10 (t, 1H), 6.88-6.82 (m, 2H), 5.95 (s, 1H), 2.99 (s, 3H), 2.75 (m, 1H), 2.59 (m, 1H), 2.45 (s, 3H), 2.09-1.81 (m, 4H).

### (R)-6-Fluoro-N-(8-fluoro-6-oxo-1,2,3,4,5,6-hexahydrophenanthridin-1-yl)-N,4-dimethyl-1H-indole-2-carboxamide (Compound 492)

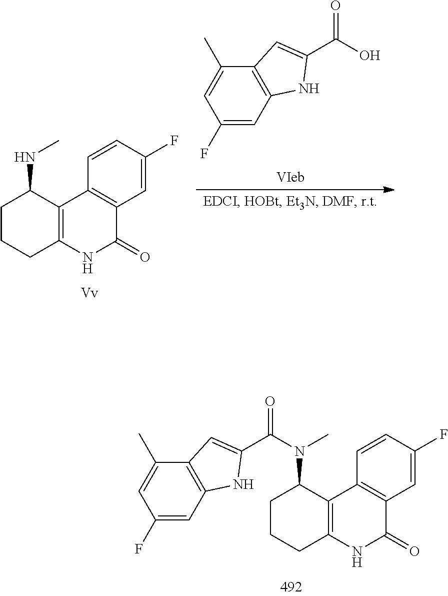

Enantiomerically pure ((R)-6-fluoro-N-(8-fluoro-6-oxo-1,2,3,4,5,6-hexahydrophenanthridin-1-yl)-N,4-dimethyl-1H-indole-2-carboxamide was synthesized in an analogous manner as described above from enantiomerically pure (R)-8-fluoro-1-(methylamino)-1,3,4,5-tetrahydrophenanthridin-6(2H)-one (Vv) and 6-fluoro-4-methyl-1H-indole-2-carboxylic acid (VIeb). LCMS m/z found 422.2 [M+H]⁺; RT=4.52 min (Method A); ¹H NMR (400 MHz, DMSO-d₆) δ 11.71 (bs, 1H), 11.52 (bs, 1H), 7.88 (m, 1H), 7.57 (m, 1H), 7.43 (m, 1H), 7.00 (d, 1H), 6.92 (s, 1H), 6.74 (d, 1H), 5.94 (s, 1H), 2.98 (s, 3H), 2.68 (m, 1H), 2.59 (m, 1H), 2.46 (s, 3H), 2.06-1.81 (m, 4H).

### (R)-4-(Difluoromethyl)-6-fluoro-N-(8-fluoro-6-oxo-1,2,3,4,5,6-hexahydrophenanthridin-1-yl)-N-methyl-1H-indole-2-carboxamide (Compound 493)

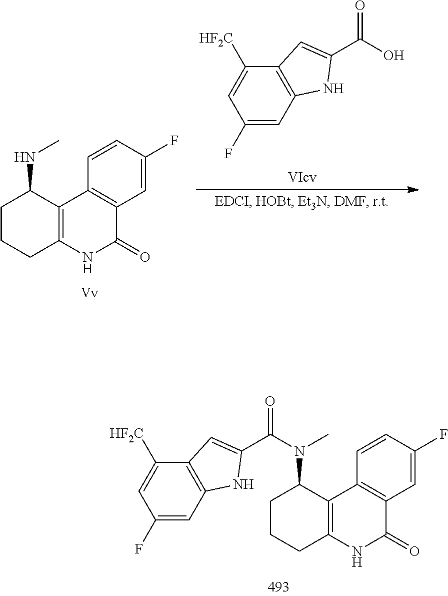

Enantiomerically pure (R)-4-(difluoromethyl)-6-fluoro-N-(8-fluoro-6-oxo-1,2,3,4,5,6-hexahydrophenanthridin-1-yl)-N-methyl-1H-indole-2-carboxamide was synthesized in an analogous manner as described above from enantiomerically pure (R)-8-fluoro-1-(methylamino)-1,3,4,5-tetrahydrophenanthridin-6(2H)-one (Vv) and 4-(difluoromethyl)-6-fluoro-1H-indole-2-carboxylic acid (VIcv). LCMS m/z found 458.1 [M+H]⁺; RT=4.41 min (Method A); ¹H NMR (400 MHz, DMSO-d₆) δ 12.13 (bs, 1H), 11.51 (bs, 1H), 7.89 (m, 1H), 7.59 (m, 1H), 7.44-7.36 (m, 2H), 7.31 (s, 1H), 7.19 (m, 1H), 7.01 (s, 1H), 5.93 (s, 1H), 2.95 (s, 3H), 2.70 (m, 1H), 2.57 (m, 1H), 2.07-1.81 (m, 4H).

### 2-Chloro-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide (Compounds 453, 454)

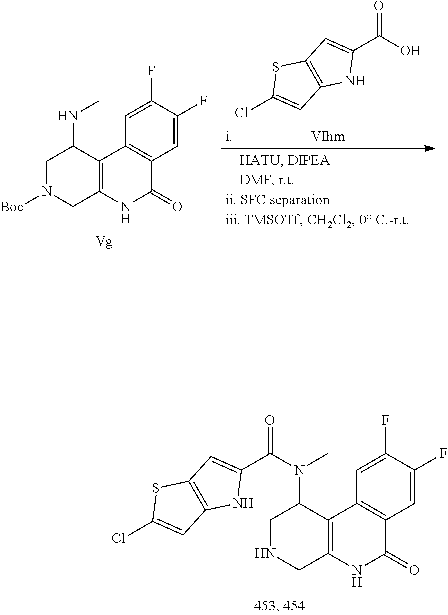 [SMILES: CNC1CN(C(=O)OC(C)(C)C)Cc2[nH]c(=O)c3cc(F)c(F)cc3c21 ; O=C(O)c1cc2sc(Cl)cc2[nH]1 ; CN(C(=O)c1cc2sc(Cl)cc2[nH]1)C1CNCc2[nH]c(=O)c3cc(F)c(F)cc3c21]

Racemic tert-butyl 1-(2-chloro-N-methyl-4H-thieno[3,2-b]pyrrole-5-carboxamido)-8,9-difluoro-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 2-chloro-4H-thieno[3,2-b]pyrrole-5-carboxylic acid (VIhm). The enantiomers were subsequently separated by preparative SFC: Method isocratic, Mobile phase methanol:CO₂—30:70. Column: Chiralpak OX-H (30×250 mm), 5 μm, flow rate: 110 g/min, and each was converted to the final product in an analogous manner as described above (for Compounds 160, 161).

Enantiomer I (Compound 453): LCMS m/z found 449.4 [M+H]⁺; RT=3.62 min (Method A); ¹H NMR (400 MHz, DMSO-d₆) δ 11.96 (bs, 1H), 11.64 (bs, 1H), 8.1 (t, 1H), 7.29-7.24 (m, 1H), 7.09 (bs, 1H), 6.89 (s, 1H), 5.67 (s, 1H), 3.82 (d, 1H), 3.66 (d, 1H), 3.15-3.08 (m, 6H); Chiral analytical SFC: RT=1.92 min, Column: Chiralpak AS-3 (4.6×150 mm) 3 μm, 40% (0.5% DEA in Methanol), Flow rate: 3.0 g/min.

Enantiomer II (Compound 454): LCMS m/z found 449.1 [M+H]⁺; RT=3.61 min (Method A); ¹H NMR (400 MHz, DMSO-d₆) δ 11.96 (bs, 1H), 11.64 (bs, 1H), 8.1 (t, 1H), 7.29-7.24 (m, 1H), 7.09 (bs, 1H), 6.89 (s, 1H), 5.67 (s, 1H), 3.82 (d, 1H), 3.66 (d, 1H), 3.15-3.08 (m, 6H); Chiral analytical SFC: RT=2.95 min, Column: Chiralpak AS-3 (4.6×150 mm) 3 μm, 30% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-N,4-dimethyl-1H-indole-2-carboxamide (Compounds 456, 457)

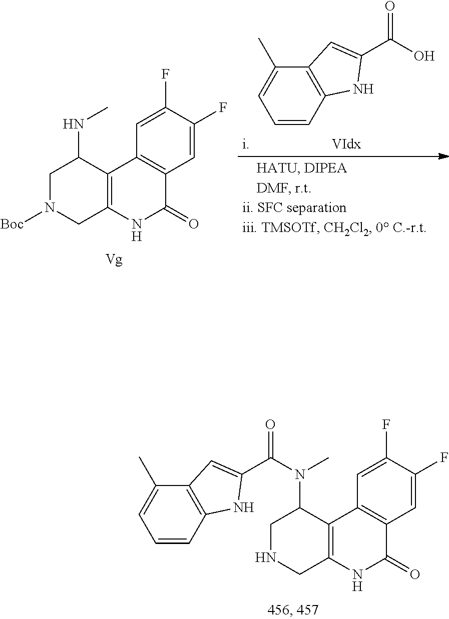

Racemic tert-butyl 1-(N,4-dimethyl-1H-indole-2-carboxamido)-8,9-difluoro-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 4-methyl-1H-indole-2-carboxylic acid (VIdx). The enantiomers were subsequently separated by preparative SFC: Method isocratic, Mobile phase methanol:CO₂—30:70. Column: Chiralpak OX-H (30×250 mm), 5 μm, flow rate: 110 g/min, and each was converted to the final product in an analogous manner as described above (for Compounds 160, 161).

Enantiomer I (Compound 456): LCMS m/z found 423.2 [M+H]⁺; RT=3.45 min (Method A); ¹H NMR (400 MHz, DMSO-d₆) δ 11.66 (m, 2H), 8.13 (t, 1H), 7.29 (d, 2H), 7.10 (t, 1H), 6.94 (s, 1H), 6.83 (d, 1H), 5.81 (bs, 1H), 3.98 (d, 1H), 3.79 (d, 1H), 3.3-3.05 (m, 5H), 2.47 (s, 3H); Chiral analytical SFC: RT=1.8 min, Column: Chiralpak AS-3 (4.6×150 mm) 3 m, 30% (0.5% DEA in Methanol), Flow rate: 3.0 g/min.

Enantiomer II (Compound 457): LCMS m/z found 423.2 [M+H]⁺; RT=3.44 min (Method A); ¹H NMR (400 MHz, DMSO-d₆) δ 11.66 (m, 2H), 8.13 (t, 1H), 7.29 (d, 2H), 7.10 (t, 1H), 6.94 (s, 1H), 6.83 (d, 1H), 5.81 (bs, 1H), 3.98 (d, 1H), 3.79 (d, 1H), 3.3-3.05 (m, 5H), 2.47 (s, 3H); Chiral analytical SFC: RT=2.57 min, Column: Chiralpak AS-3 (4.6×150 mm) 3 μm, 30% (0.5% DEA in Methanol), Flow rate: 3.0 g/min.

### N-(8,9-difluoro-6-oxo-1,2,3,4,5,6-hexahydrobenzo[c][1,7]naphthyridin-1-yl)-6-fluoro-N,4-dimethyl-1H-indole-2-carboxamide (Compounds 458, 459)

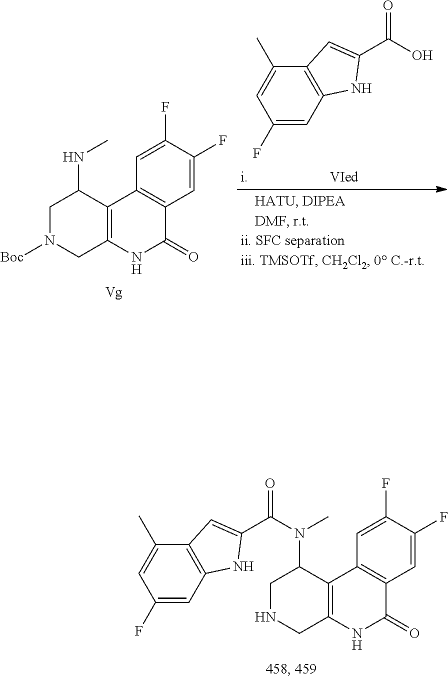

Racemic tert-butyl 8,9-difluoro-1-(6-fluoro-N,4-dimethyl-1H-indole-2-carboxamido)-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 6-fluoro-4-methyl-1H-indole-2-carboxylic acid (VIeb). The enantiomers were subsequently separated by preparative SFC: Method isocratic, Mobile phase methanol:CO₂—40:60. Column: Chiralpak IC (30×250 mm) 5 μm, flow rate: 100 g/min, and each was converted to the final product in an analogous manner as described above (for Compounds 160, 161).

Enantiomer I (Compound 458): LCMS m/z found 441.2 [M+H]⁺; RT=3.61 min (Method A); ¹H NMR (400 MHz, DMSO-d₆) δ 11.74 (bs, 1H), 11.64 (bs, 1H), 8.11 (t, 1H), 7.31-7.27 (m, 1H), 7.00-6.95 (m, 2H), 6.74 (d, 1H), 5.69 (s, 1H), 3.81 (d, 1H), 3.66 (d, 1H), 3.17 (bs, 5H), 2.79 (bs, 1H), 2.47 (s, 3H). Chiral analytical SFC: RT=1.53 min, Column: Chiralpak AS-3 (4.6×150 mm) 3 μm, 30% (0.5% DEA in Methanol), Flow rate: 3.0 g/min.

Enantiomer II (Compound 459): LCMS m/z found 441.2 [M+H]⁺; RT=3.60 min (Method A); ¹H NMR (400 MHz, DMSO-d₆) δ 11.74 (bs, 1H), 11.64 (bs, 1H), 8.11 (t, 1H), 7.31-7.27 (m, 1H), 7.00-6.95 (m, 2H), 6.74 (d, 1H), 5.69 (s, 1H), 3.81 (d, 1H), 3.66 (d, 1H), 3.17 (bs, 5H), 2.79 (bs, 1H), 2.47 (s, 3H). Chiral analytical SFC: RT=1.96 min, Column: Chiralpak AS-3 (4.6×150 mm) 3 μm, 30% (0.5% DEA in Methanol), Flow rate: 3.0 g/min.

### 4-Chloro-N-(8,9-difluoro-6-oxo-1,2,3,4,5,6-hexahydrobenzo[c][1,7]naphthyridin-1-yl)-6-fluoro-N-methyl-1H-indole-2-carboxamide (Compounds 460, 461)

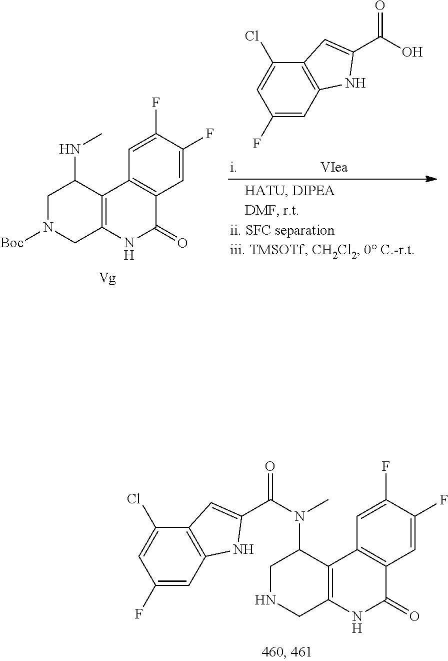 [SMILES: CNC1CN(C(=O)OC(C)(C)C)Cc2[nH]c(=O)c3cc(F)c(F)cc3c21 ; O=C(O)c1cc2c(Cl)cc(F)cc2[nH]1 ; CN(C(=O)c1cc2c(Cl)cc(F)cc2[nH]1)C1CNCc2[nH]c(=O)c3cc(F)c(F)cc3c21]

Racemic tert-butyl 1-(4-chloro-6-fluoro-N-methyl-1H-indole-2-carboxamido)-8,9-difluoro-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 4-chloro-6-fluoro-1H-indole-2-carboxylic acid (VIea). The enantiomers were subsequently separated by preparative SFC: Method isocratic, Mobile phase methanol:CO₂—30:70. Column: Chiralpak OX-H (30×250 mm) 5 μm, flow rate: 100 g/min, and each was converted to the final product in an analogous manner as described above (for Compounds 160, 161).

Enantiomer I (Compound 460): LCMS m/z found 441.2 [M+H]⁺; RT=3.61 min (Method A); ¹H NMR (400 MHz, DMSO-d₆) δ 12.14 (bs, 1H), 11.68 (bs, 1H), 8.10 (t, 1H), 7.30-7.14 (m, 3H), 6.88 (s, 1H), 5.67 (s, 1H), 3.81 (d, 1H), 3.65 (d, 1H), 3.16-2.76 (m, 6H). Chiral analytical SFC: RT=1.60 min, Column: Chiralpak AS-3 (4.6×150 mm) 3 μm, 30% (0.5% DEA in Methanol), Flow rate: 3.0 g/min.

Enantiomer II (Compound 461): LCMS m/z found 441.2 [M+H]⁺; RT=3.60 min (Method A); ¹H NMR (400 MHz, DMSO-d₆) δ 12.15 (bs, 1H), 11.69 (bs, 1H), 8.10 (t, 1H), 7.30-7.14 (m, 3H), 6.88 (s, 1H), 5.67 (s, 1H), 3.81 (d, 1H), 3.65 (d, 1H), 3.16-2.76 (m, 6H). Chiral analytical SFC: RT=2.43 min, Column: Chiralpak AS-3 (4.6×150 mm) 3 μm, 30% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-N-methylbenzo[d]thiazole-5-carboxamide (Compounds 462, 463)

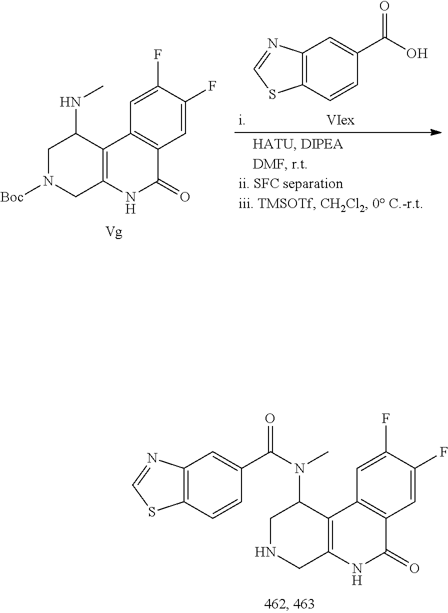 [SMILES: CNC1CN(C(=O)OC(C)(C)C)Cc2[nH]c(=O)c3cc(F)c(F)cc3c21 ; O=C(O)c1ccc2scnc2c1 ; CN(C(=O)c1ccc2scnc2c1)C1CNCc2[nH]c(=O)c3cc(F)c(F)cc3c21]

Racemic tert-butyl 8,9-difluoro-1-(N-methylbenzo[d]thiazole-5-carboxamido)-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and benzo[d]thiazole-5-carboxylic acid (VIex). The enantiomers were subsequently separated by preparative SFC: Method isocratic, Mobile phase methanol:CO₂—30:70. Column: LUX CELLOSE (30×250 mm) 5p m, flow rate: 110 g/min, and each was converted to the final product in an analogous manner as described above (for Compounds 160, 161).

Enantiomer I (Compound 462): LCMS m/z found 427.1 [M+H]⁺; RT=2.47 min (Method A); ¹H NMR (400 MHz, DMSO-d₆) δ 11.65 (bs, 1H), 9.48 (bs, 1H), 8.27 (d, 1H), 8.15-8.07 (m, 2H), 7.49-7.40 (m, 2H), 5.64 (s, 1H), 3.73 (d, 1H), 3.65 (d, 1H), 3.26-3.15 (m, 2H), 2.83-2.67 (m, 4H). Chiral analytical SFC: RT=7.46 min, Column: Chiralpak IG-3 (4.6×150 mm) 3 μm, 40% (0.5% DEA in Methanol), Flow rate: 3.0 g/min.

Enantiomer II (Compound 463): LCMS m/z found 427.2 [M+H]⁺; RT=2.49 min (Method A); ¹H NMR (400 MHz, DMSO-d₆) δ 11.62 (bs, 1H), 9.48 (bs, 1H), 8.27 (d, 1H), 8.15-8.07 (m, 2H), 7.49-7.40 (m, 2H), 5.64 (s, 1H), 3.73 (d, 1H), 3.65 (d, 1H), 3.26-3.15 (m, 2H), 2.83-2.67 (m, 4H). Chiral analytical SFC: RT=12.84 min, Column: Chiralpak IG-3 (4.6×150 mm) 3 μm, 40% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-N-methylbenzo[d]thiazole-6-carboxamide (Compounds 464, 465)

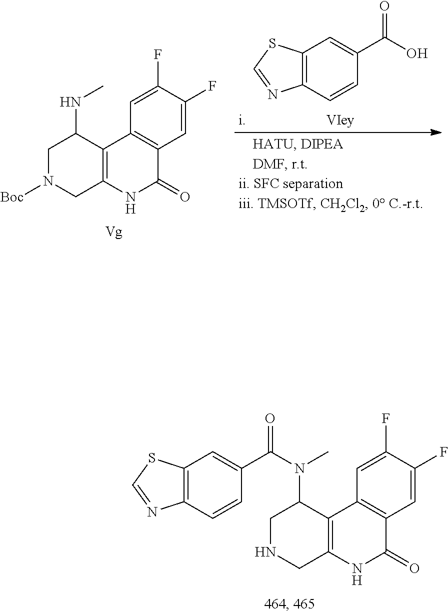 [SMILES: CNC1CN(C(=O)OC(C)(C)C)Cc2[nH]c(=O)c3cc(F)c(F)cc3c21 ; O=C(O)c1ccc2ncsc2c1 ; CN(C(=O)c1ccc2ncsc2c1)C1CNCc2[nH]c(=O)c3cc(F)c(F)cc3c21]

Racemic tert-butyl 8,9-difluoro-1-(N-methylbenzo[d]thiazole-6-carboxamido)-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and benzo[d]thiazole-6-carboxylic acid (VIey). The enantiomers were subsequently separated by preparative SFC: Method isocratic, Mobile phase methanol:CO₂—30:70. Column: ChiralPak IA (30×250 mm), 5 μm, flow rate: 110 g/min, and each was converted to the final product in an analogous manner as described above (for Compounds 160, 161).

Enantiomer I (Compound 464): LCMS m/z found 427.1 [M+H]⁺; RT=2.33 min (Method A); ¹H NMR (400 MHz, DMSO-d₆) δ 11.62 (bs, 1H), 9.48 (bs, 1H), 8.27 (d, 1H), 8.14 (t, 2H), 7.52 (d, 1H), 7.43-7.38 (m, 1H), 5.64 (s, 1H), 3.77 (d, 1H), 3.65 (d, 1H), 3.27-3.15 (m, 2H), 2.79-2.74 (m, 4H). Chiral analytical SFC: RT=1.74 min, Column: Chiralpak AD-3 (4.6×150 mm) 3 μm, 40% (0.5% DEA in Methanol), Flow rate: 3.0 g/min.

Enantiomer II (Compound 465): LCMS m/z found 427.2 [M+H]⁺; RT=2.33 min (Method A); ¹H NMR (400 MHz, DMSO-d₆) δ 11.64 (bs, 1H), 9.48 (bs, 1H), 8.27 (d, 1H), 8.15-8.1 (m, 2H), 7.52 (d, 1H), 7.43-7.38 (m, 1H), 5.63 (s, 1H), 3.77 (d, 1H), 3.65 (d, 1H), 3.27-3.15 (m, 2H), 2.79-2.74 (m, 4H). Chiral analytical SFC: RT=3.88 min, Column: Chiralpak AD-3 (4.6×150 mm) 3 μm, 40% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-N-methyl-2,3-dihydro-1H-indene-5-carboxamide (Compounds 471, 472)

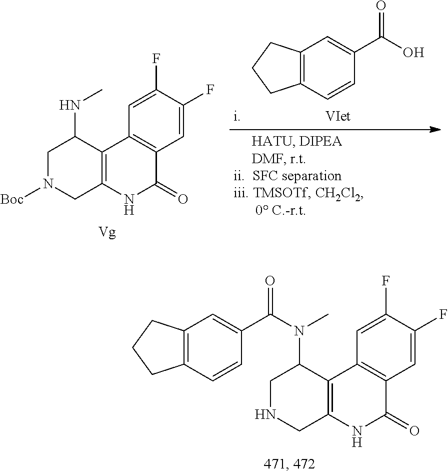 [SMILES: CNC1CN(C(=O)OC(C)(C)C)Cc2[nH]c(=O)c3cc(F)c(F)cc3c21 ; O=C(O)c1ccc2c(c1)CCC2 ; CN(C(=O)c1ccc2c(c1)CCC2)C1CNCc2[nH]c(=O)c3cc(F)c(F)cc3c21]

Racemic tert-butyl 8,9-difluoro-1-(N-methyl-2,3-dihydro-1H-indene-5-carboxamido)-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 2,3-dihydro-1H-indene-5-carboxylic acid (VIet). The enantiomers were subsequently separated by preparative SFC: Method isocratic, Mobile phase methanol:CO₂—40:60. Column: Chiralcel OX-3 (30×250 mm), 5 μm, flow rate: 110 g/min, and each was converted to the final product in an analogous manner as described above (for Compounds 160, 161).

Enantiomer I (Compound 471): LCMS m/z found 410.1 [M+H]⁺; RT=3.31 min (Method A); ¹H NMR (400 MHz, DMSO-d₆) δ 11.60 (bs, 1H), 8.13 (t, 1H), 7.38-7.33 (m, 1H), 7.28 (d, 1H), 7.21 (s, 1H), 7.09 (d, 1H), 5.59 (s, 1H), 3.75 (d, 1H), 3.63 (d, 1H), 3.22 (d, 1H), 3.15-3.11 (m, 1H), 2.87 (t, 4H), 2.69 (s, 3H), 2.03 (t, 2H). Chiral analytical SFC: RT=2.25 min, Column: Chiralpak AD-3 (4.6×150 mm) 3 μm, 30% (0.5% DEA in Methanol), Flow rate: 3.0 g/min.

Enantiomer II (Compound 472): LCMS m/z found 410.1 [M+H]⁺; RT=3.31 min (Method A); ¹H NMR (400 MHz, DMSO-d₆) δ 11.60 (bs, 1H), 8.11 (t, 1H), 7.38-7.33 (m, 1H), 7.28 (d, 1H), 7.21 (s, 1H), 7.09 (d, 1H), 5.59 (s, 1H), 3.75 (d, 1H), 3.63 (d, 1H), 3.22 (d, 1H), 3.15-3.11 (m, 1H), 2.87 (t, 4H), 2.69 (s, 3H), 2.03 (t, 2H) Chiral analytical SFC: RT=4.41 min, Column: Chiralpak AD-3 (4.6×150 mm) 3 μm, 30% (0.5% DEA in Methanol), Flow rate: 3.0 g/min.

### N-(8,9-difluoro-6-oxo-1,2,3,4,5,6-hexahydrobenzo[c][1,7]naphthyridin-1-yl)-N-methyl-1H-indazole-5-carboxamide (Compounds 473, 474)

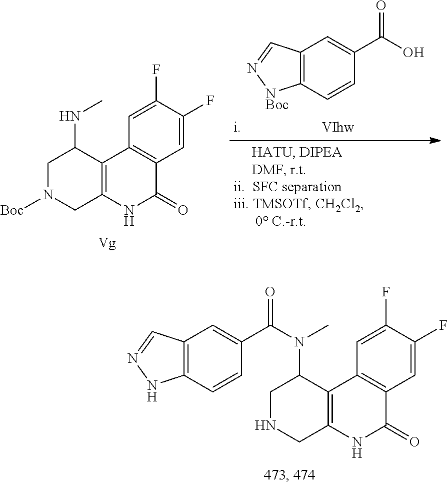 [SMILES: CNC1CN(C(=O)OC(C)(C)C)Cc2[nH]c(=O)c3cc(F)c(F)cc3c21 ; CC(C)(C)OC(=O)n1ncc2cc(C(=O)O)ccc21 ; CN(C(=O)c1ccc2[nH]ncc2c1)C1CNCc2[nH]c(=O)c3cc(F)c(F)cc3c21]

Racemic tert-butyl 1-(1-(tert-butoxycarbonyl)-N-methyl-1H-indazole-5-carboxamido)-8,9-difluoro-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 1-(tert-butoxycarbonyl)-1H-indazole-5-carboxylic acid (VIhw). The enantiomers were subsequently separated by preparative SFC: Method isocratic, Mobile phase methanol:CO₂—40:60. Column: Chiralcel OX-3 (30×250 mm) 5 μm, flow rate: 110 g/min, and each was converted to the final product in an analogous manner as described above (for Compounds 160, 161).

Enantiomer I (Compound 473): LCMS m/z found 410.1 [M+H]⁺; RT 1.90 min (Method A); ¹H NMR (400 MHz, DMSO-d₆) δ 13.28 (bs, 1H), 11.66 (bs, 1H), 8.19-8.15 (m, 2H), 7.88 (s, 1H), 7.65 (d, 1H), 7.49-7.40 (m, 2H), 5.68 (s, 1H), 3.81 (d, 1H), 3.69 (d, 1H), 3.31-3.2 (m, 2H), 2.81 (s, 4H). Chiral analytical SFC: RT=2.04 min, Column: Chiralpak IG-3 (4.6×150 mm) 3 μm, 40% (0.5% DEA in Methanol), Flow rate: 3.0 g/min.

Enantiomer II (Compound 474): LCMS m/z found 410.1 [M+H]⁺; RT=1.92 min (Method A); ¹H NMR (400 MHz, DMSO-d₆) δ 13.23 (bs, 1H), 11.61 (bs, 1H), 8.15-8.10 (m, 2H), 7.83 (s, 1H), 7.60 (d, 1H), 7.44-7.34 (m, 2H), 5.63 (s, 1H), 3.76 (d, 1H), 3.64 (d, 1H), 3.25-3.14 (m, 2H), 2.76 (s, 4H). Chiral analytical SFC: RT=3.15 min, Column: Chiralpak IG-3 (4.6×150 mm) 3 μm, 40% (0.5% DEA in Methanol), Flow rate: 3.0 g/min.

### N-(8-Fluoro-6-oxo-1,4,5,6-tetrahydro-2H-pyrano[3,4-c]isoquinolin-1-yl)-N-methyl-1H-indazole-6-carboxamide (Compounds 469, 470)

### 1-(tert-Butoxycarbonyl)-1H-indazole-6-carboxylic acid (VIhu)

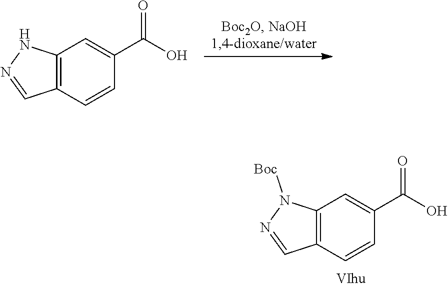 [SMILES: O=C(O)c1ccc2cn[nH]c2c1 ; CC(C)(C)OC(=O)n1ncc2ccc(C(=O)O)cc21]

To a stirred solution of 1H-indazole-6-carboxylic acid (0.20 g, 1.23 mmol) in 5 mL 1,4-dioxane/water (1:1) was added NaOH (58 mg, 2.46 mmol) at room temperature. After 10 minutes at room temperature, of di-tert-butyl dicarbonate (0.54 mL, 2.46 mmol) was added. After 5 h, the reaction mixture was quenched with water (100 mL). The mixture was extracted with EtOAc (2×200 mL). The combined organics were washed with brine (200 mL), dried over anhydrous Na₂SO₄ and concentrated under reduced pressure. The obtained crude product was triturated with n-pentane (10 mL) at room temperature, filtered, and dried under vacuum to afford 1-(tert-butoxycarbonyl)-1H-indazole-6-carboxylic acid (VIhu, 0.28 g, 86%) as an off white solid. LCMS m/z found 263.2 [M+H]⁺.

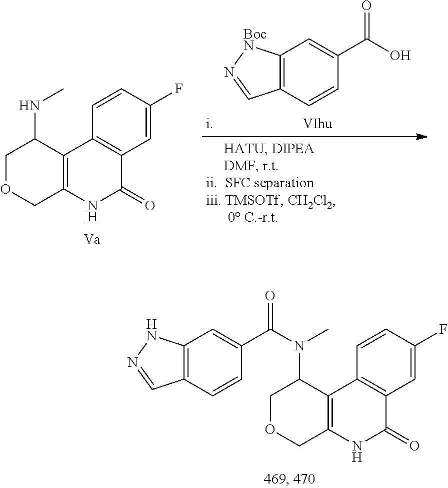 [SMILES: CNC1COCc2[nH]c(=O)c3cc(F)ccc3c21 ; CC(C)(C)OC(=O)n1ncc2ccc(C(=O)O)cc21 ; CN(C(=O)c1ccc2cn[nH]c2c1)C1COCc2[nH]c(=O)c3cc(F)ccc3c21]

Racemic tert-butyl 6-((8-fluoro-6-oxo-1,4,5,6-tetrahydro-2H-pyrano[3,4-c]isoquinolin-1-yl)(methyl)carbamoyl)-1H-indazole-1-carboxylate was synthesized in an analogous manner as described above from 8-fluoro-1-(methylamino)-1,5-dihydro-2H-pyrano[3,4-c]isoquinolin-6(4H)-one (Vg) and 1-(tert-butoxycarbonyl)-1H-indazole-6-carboxylic acid (VIhu). The enantiomers were subsequently separated by preparative SFC: Method isocratic, Mobile phase methanol:CO₂—40:60. Column: Chiralpak IC-3 (30×250 mm) 5 μm, flow rate: 110 g/min, followed by Boc deprotection of the separated enantiomers in an analogous manner as described above.

Enantiomer I (Compound 469): LCMS m/z found 393.0 [M+H]⁺; RT=2.58 min (Method A); ¹H NMR (400 MHz, DMSO-d₆) 13.22 (bs, 1H), 11.61 (bs, 1H), 8.2 (s, 1H), 7.93-7.91 (m, 1H), 7.84-7.76 (m, 2H), 7.69-7.66 (m, 1H), 7.60 (s, 1H), 7.09 (d, 1H), 5.73 (s, 1H), 4.59 (d, 1H), 4.47 (d, 1H), 4.29 (d, 1H), 4.08-4.04 (m, 1H), 2.69 (s, 3H). Chiral analytical SFC: RT=1.81 min, Column: Chiralpak AD-H (4.6×150 mm) 3 μm, 50% (0.5% DEA in Methanol), Flow rate: 3.0 g/min.

Enantiomer II (Compound 470): LCMS m/z found 393.1 [M+H]⁺; RT=2.61 min (Method A); ¹H NMR (400 MHz, DMSO-d₆) δ 13.22 (bs, 1H), 11.61 (bs, 1H), 8.2 (s, 1H), 7.93-7.91 (m, 1H), 7.84-7.77 (m, 2H), 7.69-7.66 (m, 1H), 7.53 (s, 1H), 7.09 (d, 1H), 5.73 (s, 1H), 4.58 (d, 1H), 4.47 (d, 1H), 4.29 (d, 1H), 4.08-4.04 (m, 1H), 2.69 (s, 3H). Chiral analytical SFC: RT=1.81 min, Column: Chiralpak AD-3 (4.6×150 mm) 3 μm, 50% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-N-methyl-1H-indazole-6-carboxamide (Compounds 495, 496)

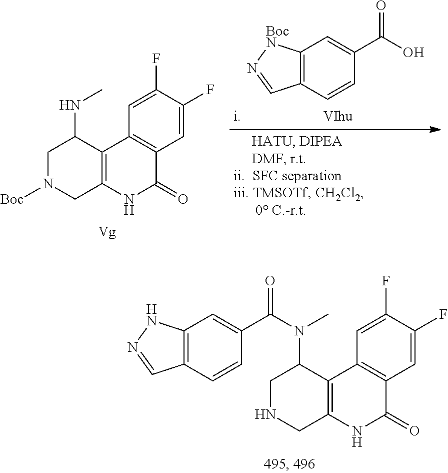 [SMILES: CNC1CN(C(=O)OC(C)(C)C)Cc2[nH]c(=O)c3cc(F)c(F)cc3c21 ; CC(C)(C)OC(=O)n1ncc2ccc(C(=O)O)cc21 ; CN(C(=O)c1ccc2cn[nH]c2c1)C1CNCc2[nH]c(=O)c3cc(F)c(F)cc3c21]

Racemic tert-butyl 1-(1-(tert-butoxycarbonyl)-N-methyl-1H-indazole-6-carboxamido)-8,9-difluoro-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 1-(tert-butoxycarbonyl)-1H-indazole-6-carboxylic acid (VIhu). The enantiomers were subsequently separated by preparative SFC: Method isocratic, Mobile phase methanol:CO₂—40:60. Column: Chiralpak IC-3 (30×250 mm), 5μ, flow rate: 110 g/min, and each was converted to the final product in an analogous manner as described above (for Compounds 160, 161).

Enantiomer I (Compound 495): LCMS m/z found 410.1 [M+H]⁺; RT 2.24 min (Method A); ¹H NMR (400 MHz, DMSO-d₆) δ 13.27 (bs, 1H), 11.67 (bs, 1H), 8.13 (t, 2H), 7.84 (d, 1H), 7.5 (s, 1H), 7.44-7.39 (m, 1H), 7.07 (d, 1H), 5.64 (s, 1H), 3.78-3.62 (m, 2H), 3.28-3.64 (d, 2H), 2.77 (bs, 1H), 2.71 (s, 3H). Chiral analytical SFC: RT=4.42 min, Column: Chiralpak AS-H (4.6×150 mm) 3 μm, 40% (0.5% DEA in Methanol), Flow rate: 3.0 g/min.

Enantiomer II (Compound 496): LCMS m/z found 410.1 [M+H]⁺; RT=2.23 min (Method A); ¹H NMR (400 MHz, DMSO-d₆) δ 13.20 (bs, 1H), 11.62 (bs, 1H), 8.13 (t, 2H), 7.84 (d, 1H), 7.5 (s, 1H), 7.44-7.39 (m, 1H), 7.07 (d, 1H), 5.64 (s, 1H), 3.78-3.62 (m, 2H), 3.28-3.61 (d, 2H), 2.78 (bs, 1H), 2.71 (s, 3H). Chiral analytical SFC: RT=1.85 min, Column: Chiralpak AS-H (4.6×150 mm) 3 μm, 40% (0.5% DEA in Methanol), Flow rate: 3.0 g/min.

### 2-Fluoro-4-((2-(trimethylsilyl)ethoxy)methyl)-4H-thieno[3,2-b]pyrrole-5-carboxylic acid

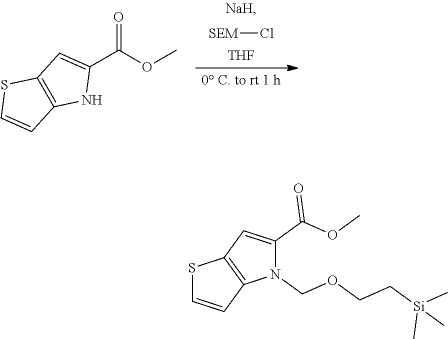 [SMILES: COC(=O)c1cc2sccc2[nH]1 ; C[Si](C)(C)CCOCCl ; COC(=O)c1cc2sccc2n1COCC[Si](C)(C)C ; "THF"]

Step i: To a stirred 0° C. solution of methyl 4H-thieno[3,2-b]pyrrole-5-carboxylate (10 g, 52.2 mmol) in 100 mL of THF was added NaH (60% in mineral oil, 6.6 g, 166 mmol). After addition, the reaction was stirred at room temperature for 10 min followed by the addition of 2-(trimethylsilyl)ethoxymethyl chloride (14.6 mL, 82.3 mmol). The reaction mixture was allowed to stir at room temperature for 1 h. The reaction mixture was quenched with water (100 mL) and extracted with EtOAc (2×200 mL). The combined organics were washed with brine (200 mL), dried over anhydrous Na₂SO₄ and concentrated under reduced pressure. The obtained crude product was triturated with n-pentane (50 mL) at room temperature, filtered, dried under vacuum to afford methyl 4-((2-(trimethylsilyl)ethoxy)methyl)-4H-thieno[3,2-b]pyrrole-5-carboxylate (10 g, 75%) as an off-white solid. LCMS: m/z found 312.28 [M+H]⁺.

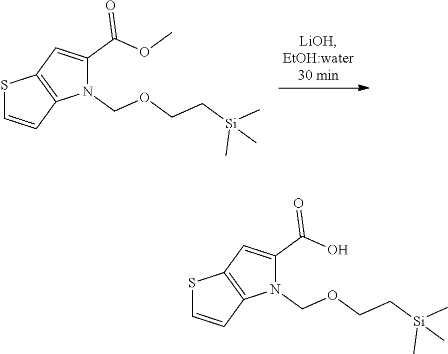 [SMILES: COC(=O)c1cc2sccc2n1COCC[Si](C)(C)C ; C[Si](C)(C)CCOCn1c(C(=O)O)cc2sccc21]

Step ii: To a stirred solution of methyl 4-((2-(trimethylsilyl)ethoxy)methyl)-4H-thieno[3,2-b]pyrrole-5-carboxylate (10 g, 32.1 mmol) in 100 mL of EtOH was added LiOH (3.8 g, 160.5 mmol). After addition, the reaction was stirred at room temperature for 30 min. The reaction mixture was neutralized with 1 M HCl (50 mL) and the aqueous layer was extracted with 10% methanol in dichloromethane (2×20 mL). The combined organic layers were washed with brine (20 mL), dried over anhydrous Na₂SO₄ and concentrated under reduced pressure. The obtained crude product was triturated with n-pentane (50 mL) at room temperature, filtered, dried under vacuum to afford (4-((2-(trimethylsilyl)ethoxy)methyl)-4H-thieno[3,2-b]pyrrole-5-carboxylic acid (8 g, 83%) as an off white solid. LCMS: m/z found 298.44 [M+H]⁺.

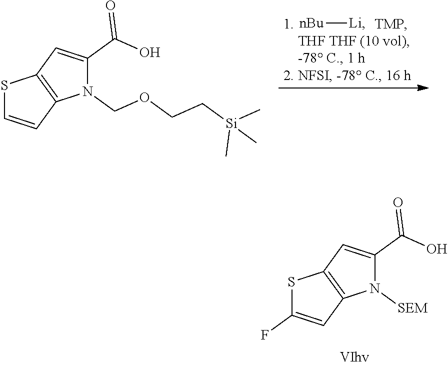

Step iii: To a stirred solution of 4-((2-(trimethylsilyl)ethoxy)methyl)-4H-thieno[3,2-b]pyrrole-5-carboxylic acid (6.0 g, 20.17 mmol) and 2,2,6,6-tetramethylpiperidine (7.6 mL, 40.34 mmol) in 60 mL of dry THE at −78° C. was added n-BuLi (2.5M solution in hexane, 16.13 mmol, 40.34 mmol) dropwise. The reaction mixture was stirred at −78° C. for 30 min. To the reaction mixture was added N-fluorobenzenesulfonimide (7.6 g, 24.2 mmol) at −78° C. The mixture was allowed to warm to RT and stirred for 16 h. The reaction mixture was diluted with water (100 mL) and extracted with ethyl acetate (2×100 mL). The combined organic layers were dried over anhydrous Na₂SO₄ and concentrated under reduced pressure. The crude product was purified by reverse phase semi-prep MPLC to afford 2-fluoro-4-((2-(trimethylsilyl)ethoxy)methyl)-4H-thieno[3,2-b]pyrrole-5-carboxylic acid (VIhv, 1.8 g, 28%) as an off-white solid. LCMS: m/z found 324.29 [M+H]+, ¹H NMR (400 MHz, DMSO-d₆): 12.55 (bs, 1H), 7.19 (s, 1H), 7.17 (d, 1H), 5.81 (s, 2H), 3.44 (t, 2H), 0.77 (t, 2H), 0.18 (s, 9H).

### 2-Fluoro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide (Compounds 497, 498)

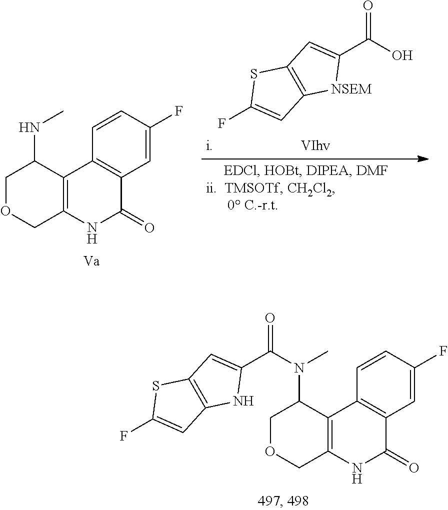

Step i: To a stirred solution of 2-fluoro-4-((2-(trimethylsilyl)ethoxy)methyl)-4H-thieno[3,2-b]pyrrole-5-carboxylic acid (VIhv, 305 mg, 0.96 mmol) in 1 mL of DMF at room temperature were added DIPEA (0.45 mL, 2.42 mmol), EDC. HCl (231 mg, 1.2 mmol), and HOBt (164 mg, 1.2 mmol). After stirring for 15 min at room temperature, racemic 8-fluoro-1-(methylamino)-1,5-dihydro-2H-pyrano[3,4-c]isoquinolin-6(4H)-one (Va, 0.20 g, 0.81 mmol) was added. After stirring at room temperature for 16 h, the reaction mixture was poured into ice cold water (10 mL), stirred for 30 min. The resulting precipitate was collected by filtration, washed with water, and dried under vacuum to provide 150 mg (0.27 mmol, 34%) 2-fluoro-N-(8-fluoro-6-oxo-1,4,5,6-tetrahydro-2H-pyrano[3,4-c]isoquinolin-1-yl)-N-methyl-4-((2-(trimethylsilyl)ethoxy)methyl)-4H-thieno[3,2-b]pyrrole-5-carboxamide (150 mg, 0.27 mmol).

LCMS: m/z found 546.16 [M+H]⁺.

Step ii: To the solution of 2-fluoro-N-(8-fluoro-6-oxo-1,4,5,6-tetrahydro-2H-pyrano[3,4-c]isoquinolin-1-yl)-N-methyl-4-((2-(trimethylsilyl)ethoxy)methyl)-4H-thieno[3,2-b]pyrrole-5-carboxamide (200 mg, 0.36 mmol) in DCM (1 mL) at −15° C. was added TMSOTf (0.16 mL, 0.73 mmol). The resulting reaction mixture was stirred at −15° C. for 30 min.

The reaction was quenched with 10% saturated Na₂CO₃ solution (10 mL) and extracted with EtOAc (3×50 mL). The combined organics were dried over sodium sulfate and concentrated. The residue was triturated with diethyl ether (10 mL) and filtered. The solid was dried under vacuum to afford 2-fluoro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide (13 mg, 67%) as an off-white solid. LCMS: m/z found 416.32 [M+H]⁺. The enantiomers were subsequently separated by preparative SFC: Method isocratic, Mobile phase methanol:CO₂—40:60. Column: Chiralpak IA-3 (30×250 mm) 5 μm, flow rate: 110 g/min.

Enantiomer I (Compound 497): LCMS m/z found 416.0 [M+H]⁺; RT 3.97 min (Method A); ¹H NMR (400 MHz, DMSO-d₆) δ 11.93 (bs, 1H), 11.62 (bs, 1H), 7.90-7.88 (dd, 1H), 7.64-7.59 (m, 1H), 7.50 (bs, 1H), 6.88 (s, 1H), 6.78 (bs, 1H), 5.71 (s, 1H), 4.62 (d, 1H), 4.46 (d, 1H), 4.12-4.01 (m, 2H), 3.09 (s, 3H). Chiral analytical SFC: RT=6.44 min, Column: Chiralpak AS-H (4.6×150 mm) 3 μm, 40% (0.5% DEA in Methanol), Flow rate: 3.0 g/min.

Enantiomer II (Compound 498): LCMS m/z found 416.0 [M+H]⁺; RT=3.97 min (Method A); ¹H NMR (400 MHz, DMSO-d₆) δ 11.94 (bs, 1H), 11.62 (bs, 1H), 7.91-7.88 (dd, 1H), 7.64-7.59 (m, 1H), 7.50 (bs, 1H), 6.88 (bs, 1H), 6.78 (bs, 1H), 5.71 (s, 1H), 4.62 (d, 1H), 4.47 (d, 1H), 4.09-4.04 (m, 2H), 3.09 (s, 3H). Chiral analytical SFC: RT=4.42 min, Column: Chiralpak AS-H (4.6×150 mm) 3 μm, 40% (0.5% DEA in Methanol), Flow rate: 3.0 g/min.

### N-(8,9-Difluoro-6-oxo-1,2,3,4,5,6-hexahydrobenzo[c][1,7]naphthyridin-1-yl)-2-fluoro-N-methyl-4H-thieno[3,2-b]pyrrole-5-carboxamide (Compounds 499, 500)

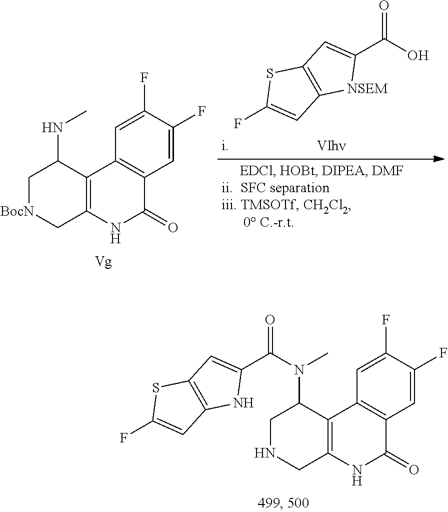

Racemic tert-butyl 8,9-difluoro-1-(2-fluoro-N-methyl-4-((2-(trimethylsilyl)ethoxy)methyl)-4H-thieno[3,2-b]pyrrole-5-carboxamido)-6-oxo-1,4,5,6-tetrahydrobenzo[c][1,7]naphthyridine-3(2H)-carboxylate was synthesized in an analogous manner as described above from tert-butyl 8,9-difluoro-1-(methylamino)-6-oxo-1,4,5,6-tetrahydrobenzo[c][1,7]naphthyridine-3(2H)-carboxylate (Vg) and 2-fluoro-4-((2-(trimethylsilyl)ethoxy)methyl)-4H-thieno[3,2-b]pyrrole-5-carboxylic acid (VIhv). The enantiomers were subsequently separated by preparative SFC: Method isocratic, Mobile phase methanol:CO₂—40:60. Column: Chiralpak IC-3 (30×250 mm) 5 μm, flow rate: 110 g/min, and each was converted to the final product in an analogous manner as described above (for Compounds 160, 161).

Enantiomer I (Compound 499): LCMS m/z found 433.0 [M+H]⁺; RT 3.27 min (Method A); ¹H NMR (400 MHz, DMSO-d₆) δ 11.86 (bs, 2H), 8.1 (t, 1H), 7.28 (bs, 1H), 6.87 (d, 1H), 6.77 (bs, 1H), 5.79 (s, 1H), 4.02 (d, 1H), 3.66 (d, 1H), 3.26-2.99 (m, 5H), 2.85 (d, 1H). Chiral analytical SFC: RT=3.26 min, Column: Chiralcel OX-3 (4.6×150 mm) 3 μm, 50% (0.2% 7 N ammonia in methanol in 1:1 Acetontrile/Methanol), Flow rate: 3.0 g/min.

Enantiomer II (Compound 500): LCMS m/z found 433.0 [M+H]⁺; RT=3.31 min (Method A); ¹H NMR (400 MHz, DMSO-d₆) δ 11.74 (bs, 2H), 8.10 (t, 1H), 7.28 (bs, 1H), 6.89-6.77 (m, 2H), 5.79 (s, 1H), 4.02 (d, 1H), 3.66 (d, 1H), 3.26-2.99 (m, 5H), 2.85 (d, 1H). Chiral analytical SFC: RT=2.36 min, Column: Chiralcel OX-3 (4.6×150 mm) 3 μm, 50% (0.2% 7 N ammonia in methanol in 1:1 Acetontrile/Methanol), Flow rate: 3.0 g/min.

### N-((1R)-8,9-difluoro-4-hydroxy-6-oxo-1,2,3,4,5,6-hexahydrophenanthridin-1-yl)-N-methyl-1H-indole-2-carboxamide (Compounds 504 and 505)

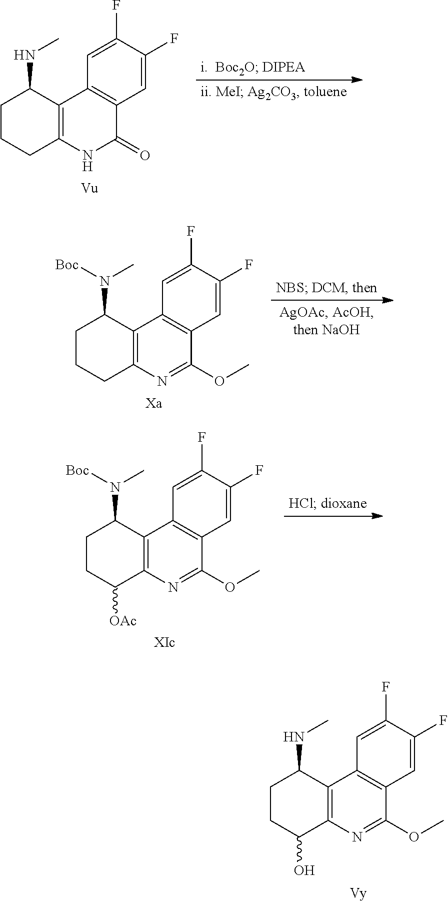 [SMILES: CN[C@@H]1CCCc2[nH]c(=O)c3cc(F)c(F)cc3c21 ; COc1nc2c(c3cc(F)c(F)cc13)[C@H](N(C)C(=O)OC(C)(C)C)CCC2 ; COc1nc2c(c3cc(F)c(F)cc13)[C@H](N(C)C(=O)OC(C)(C)C)CCC2OC(C)=O ; CN[C@@H]1CCC(O)c2nc(OC)c3cc(F)c(F)cc3c21]

Step i. To a stirred solution of (R)-8,9-difluoro-1-(methylamino)-1,3,4,5-tetrahydrophenanthridin-6(2H)-one (Vu, 0.50 g, 1.80 mmol) in 5 mL of DCM at room temperature under inert atmosphere was added DIPEA (0.99 mL, 5.60 mmol), followed by BOC anhydride (0.67 g, 2.20 mmol). The mixture was stirred for 16 h at room temperature. The reaction mixture was diluted with water (50 mL), and extracted with ethyl acetate (50 mL). The organic layer was separated and the aqueous layer was extracted with ethyl acetate (3×100 mL). The combined organics were dried over anhydrous Na₂SO₄ and concentrated under reduced pressure. The resulting crude product was triturated with diethyl ether (10 mL). The resulting precipitate was collected and dried under vacuum to provide crude tert-butyl (8,9-difluoro-6-oxo-1,2,3,4,5,6-hexahydrophenanthridin-1-yl)(methyl)carbamate (500 mg). LCMS: m/z found 365.3 [M+H]⁺.

To a stirred solution of 1.5 g (4.11 mmol, 1.0 eq.) of tert-butyl (8,9-difluoro-6-oxo-1,2,3,4,5,6-hexahydrophenanthridin-1-yl)(methyl)carbamate (1.50 g, 4.11 mmol) in 70 mL of toluene at room temperature were added Ag₂CO₃ (2.27 g, 8.2 mmol) and methyl iodide (0.89 mL, 14.04 mmol) at room temperature. The reaction mixture was stirred at 60° C. for 16 h. Upon cooling, the reaction mixture was filtered through a pad of CELITE®. The CELITE® bed was washed with EtOAc (100 mL). The combined filtrate was concentrated under reduced pressure and the crude compound was isolated by flash-chromatography (silicagel, EtOAc/hexanes, 10-20% gradient) to provide tert-butyl (8,9-difluoro-6-methoxy-1,2,3,4-tetrahydrophenanthridin-1-yl)(methyl)carbamate (Xa) (0.82 g, 52%). LCMS m/z found 379.2 [M+H]⁺; RT=1.76 min (Method E).

Step ii. To a stirred solution of tert-butyl (8,9-difluoro-6-methoxy-1,2,3,4-tetrahydrophenanthridin-1-yl)(methyl)carbamate (Xa, 0.20 g, 2.4 mmol) in 6 mL of DCM at room temperature was added N-bromosuccinimide (187 mg, 2.4 mmol) at room temperature. The reaction mixture was stirred at room temperature for 16 h. The reaction mixture was filtered through a pad of CELITE® and the CELITE® bed was washed with EtOAc (100 mL). The combined filtrate was concentrated under reduced pressure and the crude compound was isolated by flash-chromatography (silica gel, EtOAc/petroleum ether, 10-20% gradient) to provide tert-butyl (4-bromo-8,9-difluoro-6-methoxy-1,2,3,4-tetrahydrophenanthridin-1-yl)(methyl)carbamate (120 mg, 49%) as a mixture of diastereomers. LCMS m/z found 457 [M−H]⁺.

To a stirred solution of tert-butyl (4-bromo-8,9-difluoro-6-methoxy-1,2,3,4-tetrahydrophenanthridin-1-yl)(methyl)carbamate (1.2 g, 2.6 mmol) in 12 mL of acetic acid at room temperature was added silver acetate (0.43 g, 4.9 mmol) at room temperature. The reaction mixture was stirred at 80° C. for 2 h. Upon cooling, the reaction mixture was quenched with 10 N NaOH (10 mL) and extracted with DCM (100 mL). The organics were filtered and concentrated under reduced pressure. The crude compound was isolated by flash-chromatography (silicagel, EtOAc/petroleum ether, 10-20% gradient) to provide 260 mg of 1-((tert-butoxycarbonyl)(methyl)amino)-8,9-difluoro-6-methoxy-1,2,3,4-tetrahydrophenanthridin-4-yl acetate (XIc, 0.26 g, 22%). LCMS m/z found 437.3 [M+H]⁺; RT=1.76 min (Method E);

Step iii. To a stirred solution of 260 mg (0.59 mmol, 1.0 eq.) of 1-((tert-butoxycarbonyl)(methyl)amino)-8,9-difluoro-6-methoxy-1,2,3,4-tetrahydrophenanthridin-4-yl acetate (0.26 g, 0.59 mmol) in 10 mL of 1,4-dioxane at room temperature was added 4M HCl in dioxane (2.5 mL) at 0° C. Then the reaction mixture was stirred at room temperature for 16 h. The reaction mixture was concentrated under reduced pressure and the crude mixture was diluted with MeOH (10 mL). Potassium carbonate (83 mg, 0.59 mmol) was added. The mixture was stirred at room temperature for 1 h. The reaction mixture was diluted with water (50 mL), and extracted with ethyl acetate (50 mL). The organic layer was separated and the aqueous layer was extracted with ethyl acetate (3×100 mL). The combined organics were dried over anhydrous Na₂SO₄ and concentrated under reduced pressure. The crude product was triturated with diethyl ether (10 mL). The resulting precipitate was collected by filtration and dried under vacuum to afford 150 mg of 8,9-difluoro-4-hydroxy-1-(methylamino)-1,3,4,5-tetrahydrophenanthridin-6(2H)-one (Vy, 150 mg). LCMS m/z found 281.27 [M+H]⁺. RT=1.56 min (Method E);

### N-((1R)-8,9-difluoro-4-hydroxy-6-oxo-1,2,3,4,5,6-hexahydrophenanthridin-1-yl)-N-methyl-1H-indole-2-carboxamide (Compounds 504 and 505)

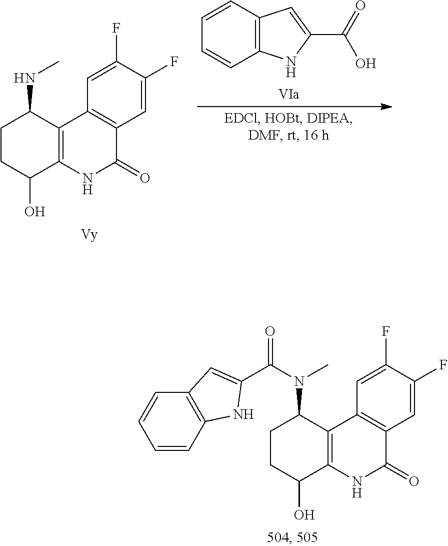

To a stirred solution 1H-indole-2-carboxylic acid (VIa, 41 mg, 0.25 mmol) in 1 mL of DMF at room temperature were added DIPEA (0.16 mL, 0.91 mmol), EDCI-HCl (63 mg, 0.32 mmol), and HOBt (45 mg, 0.33 mmol). After stirring at room temperature for 15 min, 9-difluoro-4-hydroxy-1-(methylamino)-1,3,4,5-tetrahydrophenanthridin-6(2H)-one (Vy, 60 mg, 0.22 mmol) was added. The mixture was stirred at room temperature for a further 16 h. The reaction mixture was poured into ice cold water (10 mL) and stirred for 30 minutes. The resulting precipitate was filtered, washed with water and dried under vacuum to provide N-((1R)-8,9-difluoro-4-hydroxy-6-oxo-1,2,3,4,5,6-hexahydrophenanthridin-1-yl)-N-methyl-1H-indole-2-carboxamide (45 mg, 49%) as a mixture of diastereomers.

The diastereomers were subsequently separated by preparative SFC: Method isocratic, Mobile phase methanol:CO₂—40:60. Column: Chiralpak IC-3 (30×250 mm) 5 μm, flow rate: 110 g/min.

Diastereomer I (Compound 504): LCMS m/z found 421.41 [M−H]⁻; RT=1.75 min (Method A); ¹H NMR (400 MHz, DMSO-d₆) 11.75 (bs, 2H), 8.11 (t, 1H), 7.61 (t, 1H), 7.46 (t, 1H), 7.33-7.28 (m, 1H), 7.21 (t, 1H), 7.04 (t, 1H), 6.90 (s, 1H) 5.92 (bs, 1H), 4.52 (bs, 1H), 2.98 (s, 3H), 2.07-1.88 (m, 4H). Chiral analytical SFC: RT=3.97 min, Column: Chiralpak IC-3 (4.6×150 mm) 3 μm, 40% Methanol Flow rate: 3.0 g/min.

Diastereomer II (Compound 505): LCMS m/z found 424.3 [M+H]⁺; RT=1.79 min (Method E); ¹H NMR (400 MHz, DMSO-d₆) δ 11.70 (bs, 2H), 8.12 (t, 1H), 7.61 (d, 1H), 7.47 (d, 1H), 7.34-7.29 (m, 1H), 7.21 (t, 1H), 7.04 (t, 1H), 6.90 (s, 1H) 5.93 (bs, 1H), 4.6 (t, 1H), 2.98 (s, 3H), 2.27-1.75 (m, 4H). Chiral analytical SFC: RT=5.48 min, Column: Chiralpak IC-3 (4.6×150 mm) 3 μm, 40% Methanol Flow rate: 3.0 g/min.

### N-((1R)-8,9-difluoro-4-hydroxy-6-oxo-1,2,3,4,5,6-hexahydrophenanthridin-1-yl)-5-fluoro-N-methyl-1H-indole-2-carboxamide (Compounds 501 and 502)

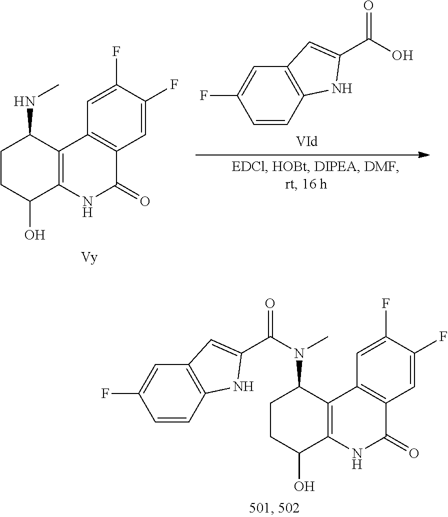

A diastereoisomeric mixture of N-((1R)-8,9-difluoro-4-hydroxy-6-oxo-1,2,3,4,5,6-hexahydrophenanthridin-1-yl)-5-fluoro-N-methyl-1H-indole-2-carboxamide was synthesized in an analogous manner as described above from a diastereoisomeric mixture of (1R)-8,9-difluoro-4-hydroxy-1-(methylamino)-1,3,4,5-tetrahydrophenanthridin-6(2H)-one (Vy) and 5-fluoro-1H-indole-2-carboxylic acid (VId). The diastereomers were subsequently separated by preparative SFC: Method isocratic, Mobile phase methanol:CO₂—40:60. Column: Chiralpak LuX Cellulose (30×250 mm) 5 μm, flow rate: 110 g/min.

Diastereomer I (Compound 501): LCMS m/z found 442.0 [M+H]⁺; RT=3.97 min (Method A); ¹H NMR (400 MHz, DMSO-d₆) 11.8 (bs, 2H), 8.11 (t, 1H), 7.48-7.44 (m, 1H), 7.36-7.23 (m, 2H), 7.07 (t, 1H), 6.88 (s, 1H), 5.90 (t, 1H), 4.60 (t, 1H), 2.93 (s, 3H), 2.07-2.33 (m, 2H), 1.85-1.75 (m, 2H). Chiral analytical SFC: RT=3.97 min, Column: Chiralpak Lux Cellulose (4.6×150 mm) 3 μm, 40% Methanol Flow rate: 3.0 g/min.

Diastereomer II (Compound 502): LCMS m/z found 442.1 [M+H]⁺; RT=3.95 min (Method A); ¹H NMR (400 MHz, DMSO-d₆) δ 11.8 (bs, 2H), 8.11 (t, 1H), 7.48-7.44 (m, 1H), 7.36-7.27 (m, 2H), 7.07 (t, 1H), 6.89 (s, 1H), 5.91 (t, 1H), 4.53 (t, 1H), 2.97 (s, 3H), 2.1-1.86 (m, 4H). Chiral analytical SFC: RT=5.48 min, Column: Chiralpak Lux Cellulose (4.6×150 mm) 3 μm, 40% Methanol Flow rate: 3.0 g/min.

### N-((1R)-8,9-Difluoro-4-hydroxy-6-oxo-1,2,3,4,5,6-hexahydrophenanthridin-1-yl)-5,6-difluoro-N-methyl-1H-indole-2-carboxamide (Compound 503)

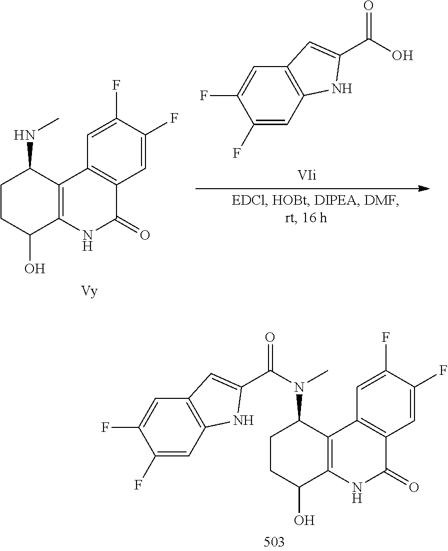

A diastereoisomeric (2:3) mixture of N-((1R)-8,9-Difluoro-4-hydroxy-6-oxo-1,2,3,4,5,6-hexahydrophenanthridin-1-yl)-5,6-difluoro-N-methyl-1H-indole-2-carboxamide was synthesized in an analogous manner as described above from a diastereoisomeric mixture of (1R)-8,9-difluoro-4-hydroxy-1-(methylamino)-1,3,4,5-tetrahydrophenanthridin-6(2H)-one (Vy) and 5,6-difluoro-1H-indole-2-carboxylic acid (VIi). LCMS: m/z found 460.0 [M+H]⁺, RT=4.15 min, (Method A); ¹H NMR (400 MHz, DMSO-d₆): 11.90 (bs, 1H), 11.30 (bs, 1H), 8.12 (t, 1H), 7.60 (t, 1H), 7.40-7.27 (m, 2H), 6.93 (bs, 1H), 5.90 (t, 1H), 5.67 (bs, 1H), 4.57 (d, 1H), 2.94 (d, 3H), 2.27-2.05 (m, 2H), 1.93-1.76 (in, 2H).

### Example 2: Biological Results

Representative compounds of the invention were tested for their abilities to inhibit formation of relaxed circular DNA (rcDNA) in a HepDE19 assay, as described elsewhere herein. Results are illustrated in Table 3.

TABLE 3

| | | DE- |
|---|---|---|
| | | 19bDNA |
| No. | Nomenclature | EC₅₀ (μM) |
| 1 | 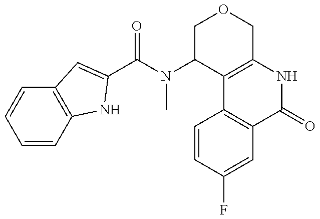 [SMILES: CN(C(=O)c1cc2ccccc2[nH]1)C1COCc2[nH]c(=O)c3cc(F)ccc3c21] | 8.6 |
| | N-(8-fluoro-6-oxo-1,4,5,6-tetrahydro-2H-pyrano[3,4-c]isoquinolin-1-yl)-N- | |
| | methyl-1H-indole-2-carboxamide | |
| | enantiomer I | |
| 2 | 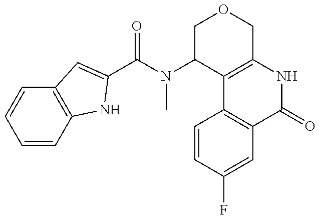 [SMILES: CN(C(=O)c1cc2ccccc2[nH]1)C1COCc2[nH]c(=O)c3cc(F)ccc3c21] | 0.02 |
| | N-(8-fluoro-6-oxo-1,4,5,6-tetrahydro-2H-pyrano[3,4-c]isoquinolin-1-yl)-N- | |
| | methyl-1H-indole-2-carboxamide | |
| | enantiomer II | |
| 4 | 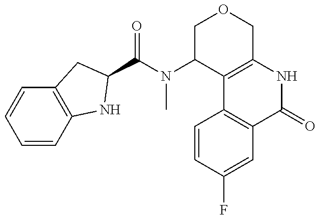 [SMILES: CN(C(=O)[C@@H]1Cc2ccccc2N1)C1COCc2[nH]c(=O)c3cc(F)ccc3c21] | 0.13 |
| | (2S)-N-(8-fluoro-6-oxo-1,4,5,6-tetrahydro-2H-pyrano[3,4-c]isoquinolin-1- | |
| | y1)-N-methylindoline-2-carboxamide | |
| | diastereoisomer II | |
| 5 | 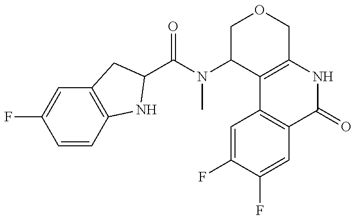 [SMILES: CN(C(=O)C1Cc2cc(F)ccc2N1)C1COCc2[nH]c(=O)c3cc(F)c(F)cc3c21] | 0.02 |
| | N-(8,9-difluoro-6-oxo-1,4,5,6-tetrahydro-2H-pyrano[3,4-c]isoquinolin-1-yl)- | |
| | 5-fluoro-N-methylindoline-2-carboxamide | |
| | diastereoisomer IA | |
| 6 | 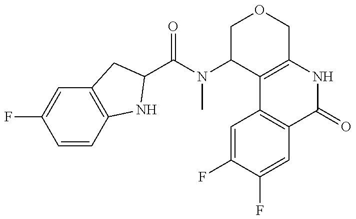 [SMILES: CN(C(=O)C1Cc2cc(F)ccc2N1)C1COCc2[nH]c(=O)c3cc(F)c(F)cc3c21] | 9.6 |
| | N-(8,9-difluoro-6-oxo-1,4,5,6-tetrahydro-2H-pyrano[3,4-c]isoquinolin-1-yl)- | |
| | 5-fluoro-N-methylindoline-2-carboxamide | |
| | diastereoisomer IIA | |
| 7 | 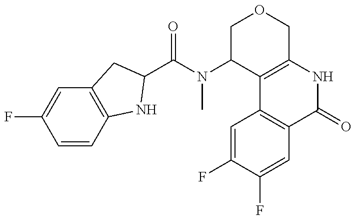 [SMILES: CN(C(=O)C1Cc2cc(F)ccc2N1)C1COCc2[nH]c(=O)c3cc(F)c(F)cc3c21] | 0.01 |
| | N-(8,9-difluoro-6-oxo-1,4,5,6-tetrahydro-2H-pyrano[3,4-c]isoquinolin-1-yl)- | |
| | 5-fluoro-N-methylindoline-2-carboxamide | |
| | diastereoisomer IB | |
| 8 | 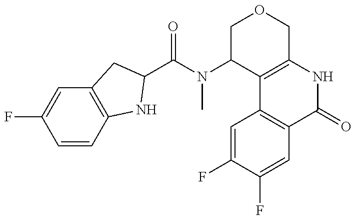 [SMILES: CN(C(=O)C1Cc2cc(F)ccc2N1)C1COCc2[nH]c(=O)c3cc(F)c(F)cc3c21] | 1.4 |
| | N-(8,9-difluoro-6-oxo-1,4,5,6-tetrahydro-2H-pyrano[3,4-c]isoquinolin-1-yl)- | |
| | 5-fluoro-N-methylindoline-2-carboxamide | |
| | diastereoisomer IIB | |
| 9 | 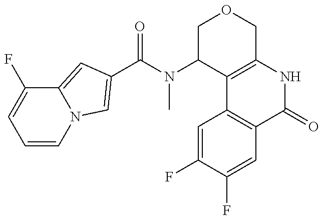 [SMILES: CN(C(=O)c1cc2c(F)cccn2c1)C1COCc2[nH]c(=O)c3cc(F)c(F)cc3c21] | 3.3 |
| | N-(8,9-difluoro-6-oxo-1,4,5,6-tetrahydro-2H-pyrano[3,4-c]isoquinolin-1-yl)- | |
| | 8-fluoro-N-methylindolizine-2-carboxamide | |
| | enantiomer I | |
| 10 | 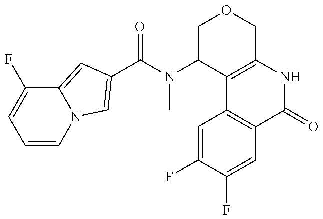 [SMILES: CN(C(=O)c1cc2c(F)cccn2c1)C1COCc2[nH]c(=O)c3cc(F)c(F)cc3c21] | 0.01 |
| | N-(8,9-difluoro-6-oxo-1,4,5,6-tetrahydro-2H-pyrano[3,4-c]isoquinolin-1-yl)- | |
| | 8-fluoro-N-methylindolizine-2-carboxamide | |
| | enantiomer II | |
| 11 | 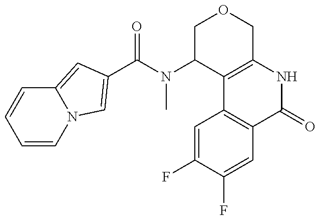 [SMILES: CN(C(=O)c1cc2ccccn2c1)C1COCc2[nH]c(=O)c3cc(F)c(F)cc3c21] | 5.3 |
| | N-(8,9-difluoro-6-oxo-1,4,5,6-tetrahydro-2H-pyrano[3,4-c]isoquinolin-1-yl)- | |
| | N-methylindolizine-2-carboxamide | |
| | enantiomer I | |
| 12 | 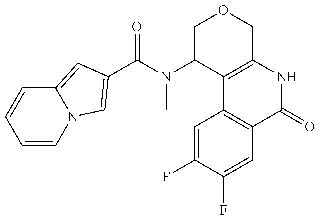 [SMILES: CN(C(=O)c1cc2ccccn2c1)C1COCc2[nH]c(=O)c3cc(F)c(F)cc3c21] | 0.01 |
| | N-(8,9-difluoro-6-oxo-1,4,5,6-tetrahydro-2H-pyrano[3,4-c]isoquinolin-1-yl)- | |
| | N-methylindolizine-2-carboxamide | |
| | enantiomer II | |
| 13 | 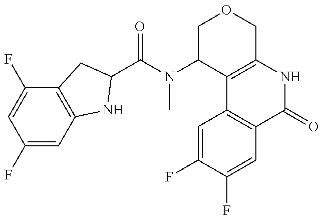 [SMILES: CN(C(=O)C1Cc2c(F)cc(F)cc2N1)C1COCc2[nH]c(=O)c3cc(F)c(F)cc3c21] | 12 |
| | N-(8,9-difluoro-6-oxo-1,4,5,6-tetrahydro-2H-pyrano[3,4-c]isoquinolin-1-yl)- | |
| | 4,6-difluoro-N-methylindoline-2-carboxamide | |
| | diastereomer Ia | |
| 14 | 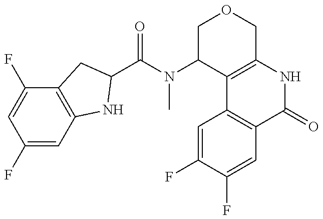 [SMILES: CN(C(=O)C1Cc2c(F)cc(F)cc2N1)C1COCc2[nH]c(=O)c3cc(F)c(F)cc3c21] | 0.04 |
| | N-(8,9-difluoro-6-oxo-1,4,5,6-tetrahydro-2H-pyrano[3,4-c]isoquinolin-1-yl)- | |
| | 4,6-difluoro-N-methylindoline-2-carboxamide | |
| | diastereomer IIa | |
| 15 | 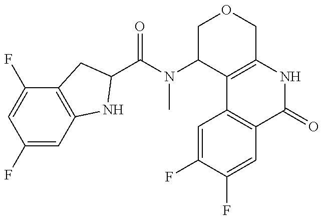 [SMILES: CN(C(=O)C1Cc2c(F)cc(F)cc2N1)C1COCc2[nH]c(=O)c3cc(F)c(F)cc3c21] | 0.96 |
| | N-(8,9-difluoro-6-oxo-1,4,5,6-tetrahydro-2H-pyrano[3,4-c]isoquinolin-1-yl)- | |
| | 4,6-difluoro-N-methylindoline-2-carboxamide | |
| | diastereomer Ib | |
| 16 | 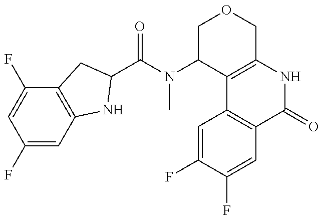 [SMILES: CN(C(=O)C1Cc2c(F)cc(F)cc2N1)C1COCc2[nH]c(=O)c3cc(F)c(F)cc3c21] | 0.02 |
| | N-(8,9-difluoro-6-oxo-1,4,5,6-tetrahydro-2H-pyrano[3,4-c]isoquinolin-1-yl)- | |
| | 4,6-difluoro-N-methylindoline-2-carboxamide | |
| | diastereomer IIb | |
| 17 | 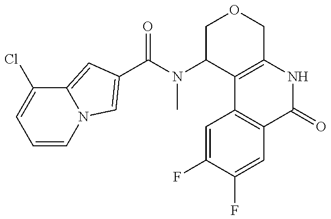 [SMILES: CN(C(=O)c1cc2c(Cl)cccn2c1)C1COCc2[nH]c(=O)c3cc(F)c(F)cc3c21] | 1.9 |
| | 8-chloro-N-(8,9-difluoro-6-oxo-1,4,5,6-tetrahydro-2H-pyrano[3,4- | |
| | c]isoquinolin-1-yl)-N-methylindolizine-2-carboxamide | |
| | enantiomer I | |
| 18 | 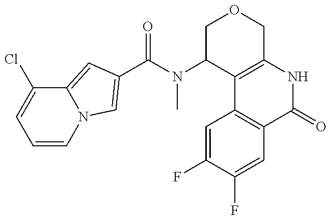 [SMILES: CN(C(=O)c1cc2c(Cl)cccn2c1)C1COCc2[nH]c(=O)c3cc(F)c(F)cc3c21] | 0.01 |
| | 8-chloro-N-(8,9-difluoro-6-oxo-1,4,5,6-tetrahydro-2H-pyrano[3,4- | |
| | c]isoquinolin-1-yl)-N-methylindolizine-2-carboxamide | |
| | enantiomer II | |
| 19 | 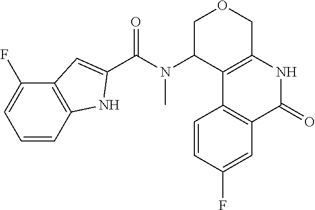 [SMILES: CN(C(=O)c1cc2c(F)cccc2[nH]1)C1COCc2[nH]c(=O)c3cc(F)ccc3c21] | 1.5 |
| | 4-fluoro-N-(8-fluoro-6-oxo-1,4,5,6-tetrahydro-2H-pyrano[3,4-c]isoquinolin- | |
| | 1-yl)-N-methyl-1H-indole-2-carboxamide | |
| | enantiomer I | |
| 20 | 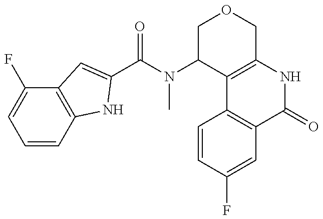 [SMILES: CN(C(=O)c1cc2c(F)cccc2[nH]1)C1COCc2[nH]c(=O)c3cc(F)ccc3c21] | 0.02 |
| | 4-fluoro-N-(8-fluoro-6-oxo-1,4,5,6-tetrahydro-2H-pyrano[3,4-c]isoquinolin- | |
| | 1-yl)-N-methyl-1H-indole-2-carboxamide | |
| | enantiomer II | |
| 21 | 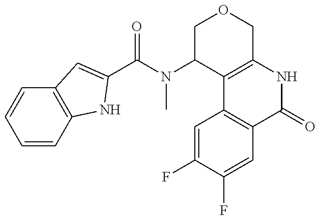 [SMILES: CN(C(=O)c1cc2ccccc2[nH]1)C1COCc2[nH]c(=O)c3cc(F)c(F)cc3c21] | 3.3 |
| | N-(8,9-difluoro-6-oxo-1,4,5,6-tetrahydro-2H-pyrano[3,4-c]isoquinolin-1-yl)- | |
| | N-methyl-1H-indole-2-carboxamide | |
| | enantiomer I | |
| 22 | 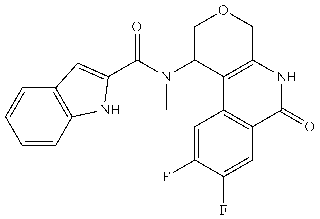 [SMILES: CN(C(=O)c1cc2ccccc2[nH]1)C1COCc2[nH]c(=O)c3cc(F)c(F)cc3c21] | 0.01 |
| | N-(8,9-difluoro-6-oxo-1,4,5,6-tetrahydro-2H-pyrano[3,4-c]isoquinolin-1-yl)- | |
| | N-methyl-1H-indole-2-carboxamide | |
| | enantiomer II | |
| 23 | 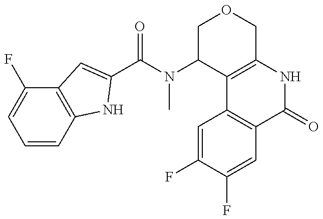 [SMILES: CN(C(=O)c1cc2c(F)cccc2[nH]1)C1COCc2[nH]c(=O)c3cc(F)c(F)cc3c21] | 4.5 |
| | N-(8,9-difluoro-6-oxo-1,4,5,6-tetrahydro-2H-pyrano[3,4-c]isoquinolin-1-yl)- | |
| | 4-fluoro-N-methyl-1H-indole-2-carboxamide | |
| | enantiomer I | |
| 24 | 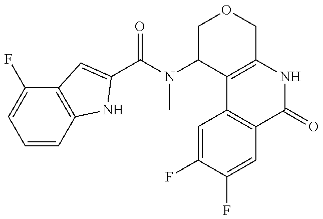 [SMILES: CN(C(=O)c1cc2c(F)cccc2[nH]1)C1COCc2[nH]c(=O)c3cc(F)c(F)cc3c21] | 0.004 |
| | N-(8,9-difluoro-6-oxo-1,4,5,6-tetrahydro-2H-pyrano[3,4-c]isoquinolin-1-yl)- | |
| | 4-fluoro-N-methyl-1H-indole-2-carboxamide | |
| | enantiomer II | |
| 25 | 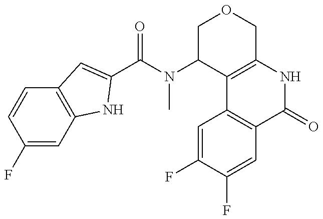 [SMILES: CN(C(=O)c1cc2ccc(F)cc2[nH]1)C1COCc2[nH]c(=O)c3cc(F)c(F)cc3c21] | 0.82 |
| | N-(8,9-difluoro-6-oxo-1,4,5,6-tetrahydro-2H-pyrano[3,4-c]isoquinolin-1-yl)- | |
| | 6-fluoro-N-methyl-1H-indole-2-carboxamide | |
| | enantiomer I | |
| 26 | 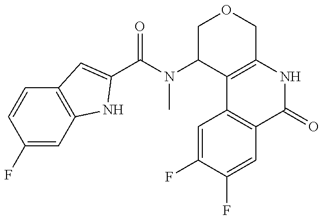 [SMILES: CN(C(=O)c1cc2ccc(F)cc2[nH]1)C1COCc2[nH]c(=O)c3cc(F)c(F)cc3c21] | 0.01 |
| | N-(8,9-difluoro-6-oxo-1,4,5,6-tetrahydro-2H-pyrano[3,4-c]isoquinolin-1-yl)- | |
| | 6-fluoro-N-methyl-1H-indole-2-carboxamide | |
| | enantiomer II | |
| 27 | 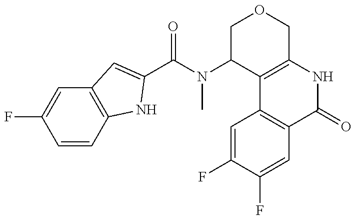 [SMILES: CN(C(=O)c1cc2cc(F)ccc2[nH]1)C1COCc2[nH]c(=O)c3cc(F)c(F)cc3c21] | 0.37 |
| | N-(8,9-difluoro-6-oxo-1,4,5,6-tetrahydro-2H-pyrano[3,4-c]isoquinolin-1-yl)- | |
| | 5-fluoro-N-methyl-1H-indole-2-carboxamide | |
| | enantiomer I | |
| 28 | 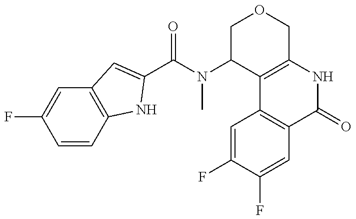 [SMILES: CN(C(=O)c1cc2cc(F)ccc2[nH]1)C1COCc2[nH]c(=O)c3cc(F)c(F)cc3c21] | 0.004 |
| | N-(8,9-difluoro-6-oxo-1,4,5,6-tetrahydro-2H-pyrano[3,4-c]isoquinolin-1-yl)- | |
| | 5-fluoro-N-methyl-1H-indole-2-carboxamide | |
| | enantiomer II | |
| 29 | 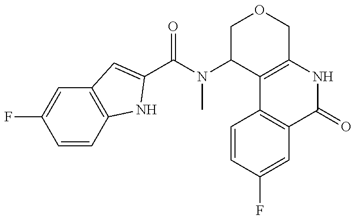 [SMILES: CN(C(=O)c1cc2cc(F)ccc2[nH]1)C1COCc2[nH]c(=O)c3cc(F)ccc3c21] | 1.5 |
| | 5-fluoro-N-(8-fluoro-6-oxo-1,4,5,6-tetrahydro-2H-pyrano[3,4-c]isoquinolin- | |
| | 1-yl)-N-methyl-1H-indole-2-carboxamide | |
| | enantiomer I | |
| 30 | 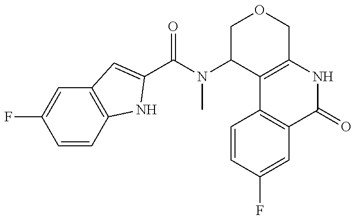 [SMILES: CN(C(=O)c1cc2cc(F)ccc2[nH]1)C1COCc2[nH]c(=O)c3cc(F)ccc3c21] | 0.01 |
| | 5-fluoro-N-(8-fluoro-6-oxo-1,4,5,6-tetrahydro-2H-pyrano[3,4-c]isoquinolin- | |
| | 1-y1)-N-methyl-1H-indole-2-carboxamide | |
| | enantiomer II | |
| 31 | 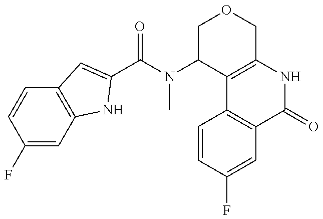 [SMILES: CN(C(=O)c1cc2ccc(F)cc2[nH]1)C1COCc2[nH]c(=O)c3cc(F)ccc3c21] | 1.2 |
| | 6-fluoro-N-(8-fluoro-6-oxo-1,4,5,6-tetrahydro-2H-pyrano[3,4-c]isoquinolin- | |
| | 1-yl)-N-methyl-1H-indole-2-carboxamide | |
| | enantiomer I | |
| 32 | 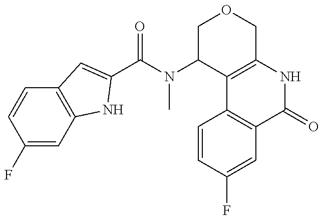 [SMILES: CN(C(=O)c1cc2ccc(F)cc2[nH]1)C1COCc2[nH]c(=O)c3cc(F)ccc3c21] | 0.02 |
| | 6-fluoro-N-(8-fluoro-6-oxo-1,4,5,6-tetrahydro-2H-pyrano[3,4-c]isoquinolin- | |
| | 1-yl)-N-methyl-1H-indole-2-carboxamide | |
| | enantiomer II | |
| 33 | 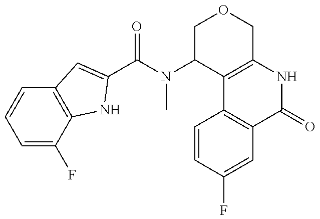 [SMILES: CN(C(=O)c1cc2cccc(F)c2[nH]1)C1COCc2[nH]c(=O)c3cc(F)ccc3c21] | 1.8 |
| | 7-fluoro-N-(8-fluoro-6-oxo-1,4,5,6-tetrahydro-2H-pyrano[3,4-c]isoquinolin- | |
| | 1-yl)-N-methyl-1H-indole-2-carboxamide | |
| | enantiomer I | |
| 34 | 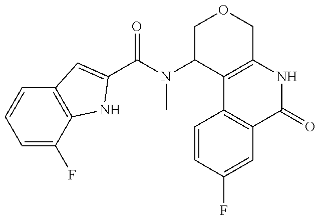 [SMILES: CN(C(=O)c1cc2cccc(F)c2[nH]1)C1COCc2[nH]c(=O)c3cc(F)ccc3c21] | 0.03 |
| | 7-fluoro-N-(8-fluoro-6-oxo-1,4,5,6-tetrahydro-2H-pyrano[3,4-c]isoquinolin- | |
| | 1-yl)-N-methyl-1H-indole-2-carboxamide | |
| | enantiomer II | |
| 35 | 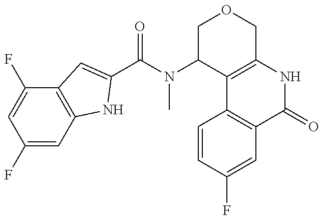 [SMILES: CN(C(=O)c1cc2c(F)cc(F)cc2[nH]1)C1COCc2[nH]c(=O)c3cc(F)ccc3c21] | 1.4 |
| | 4,6-difluoro-N-(8-fluoro-6-oxo-1,4,5,6-tetrahydro-2H-pyrano[3,4- | |
| | c]isoquinolin-1-yl)-N-methyl-1H-indole-2-carboxamide | |
| | enantiomer I | |
| 36 | 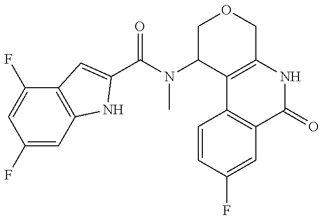 [SMILES: CN(C(=O)c1cc2c(F)cc(F)cc2[nH]1)C1COCc2[nH]c(=O)c3cc(F)ccc3c21] | 0.02 |
| | 4,6-difluoro-N-(8-fluoro-6-oxo-1,4,5,6-tetrahydro-2H-pyrano[3,4- | |
| | c]isoquinolin-1-y1)-N-methyl-1H-indole-2-carboxamide | |
| | enantiomer II | |
| 37 | 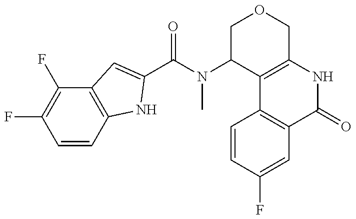 [SMILES: CN(C(=O)c1cc2c(F)c(F)ccc2[nH]1)C1COCc2[nH]c(=O)c3cc(F)ccc3c21] | 0.7 |
| | 4,5-difluoro-N-(8-fluoro-6-oxo-1,4,5,6-tetrahydro-2H-pyrano[3,4- | |
| | c]isoquinolin-1-yl)-N-methyl-1H-indole-2-carboxamide | |
| | enantiomer I | |
| 38 | 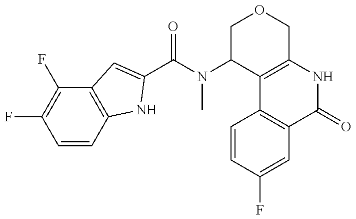 [SMILES: CN(C(=O)c1cc2c(F)c(F)ccc2[nH]1)C1COCc2[nH]c(=O)c3cc(F)ccc3c21] | 0.01 |
| | 4,5-difluoro-N-(8-fluoro-6-oxo-1,4,5,6-tetrahydro-2H-pyrano[3,4- | |
| | c]isoquinolin-1-yl)-N-methyl-1H-indole-2-carboxamide | |
| | enantiomer II | |
| 39 | 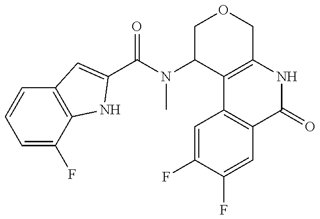 [SMILES: CN(C(=O)c1cc2cccc(F)c2[nH]1)C1COCc2[nH]c(=O)c3cc(F)c(F)cc3c21] | 0.77 |
| | N-(8,9-difluoro-6-oxo-1,4,5,6-tetrahydro-2H-pyrano[3,4-c]isoquinolin-1-yl)- | |
| | 7-fluoro-N-methyl-1H-indole-2-carboxamide | |
| | enantiomer I | |
| 40 | 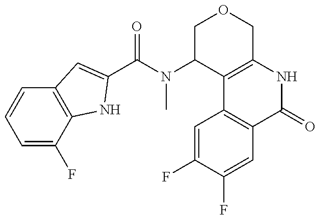 [SMILES: CN(C(=O)c1cc2cccc(F)c2[nH]1)C1COCc2[nH]c(=O)c3cc(F)c(F)cc3c21] | 0.01 |
| | N-(8,9-difluoro-6-oxo-1,4,5,6-tetrahydro-2H-pyrano[3,4-c]isoquinolin-1-yl)- | |
| | 7-fluoro-N-methyl-1H-indole-2-carboxamide | |
| | enantiomer II | |
| 41 | 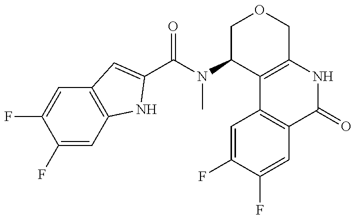 [SMILES: CN(C(=O)c1cc2cc(F)c(F)cc2[nH]1)[C@H]1COCc2[nH]c(=O)c3cc(F)c(F)cc3c21] | 2.2 |
| | (R)-N-(8,9-difluoro-6-oxo-1,4,5,6-tetrahydro-2H-pyrano[3,4-c]isoquinolin- | |
| | 1-yl)-5,6-difluoro-N-methyl-1H-indole-2-carboxamide | |
| | enantiomer I | |
| 42 | 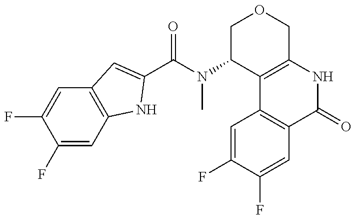 [SMILES: CN(C(=O)c1cc2cc(F)c(F)cc2[nH]1)[C@@H]1COCc2[nH]c(=O)c3cc(F)c(F)cc3c21] | 0.005 |
| | (S)-N-(8,9-Difluoro-6-oxo-1,4,5,6-tetrahydro-2H-pyrano[3,4-c]isoquinolin- | |
| | 1-yl)-5,6-difluoro-N-methyl-1H-indole-2-carboxamide | |
| | enantiomer II | |
| 43 | 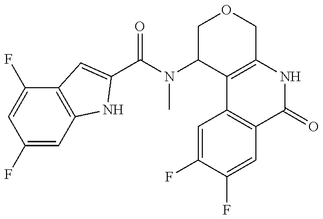 [SMILES: CN(C(=O)c1cc2c(F)cc(F)cc2[nH]1)C1COCc2[nH]c(=O)c3cc(F)c(F)cc3c21] | 1.8 |
| | N-(8,9-difluoro-6-oxo-1,4,5,6-tetrahydro-2H-pyrano[3,4-c]isoquinolin- | |
| | 1-yl)- 4,6-difluoro-N-methyl-1H-indole-2-carboxamide | |
| | enantiomer I | |
| 44 | 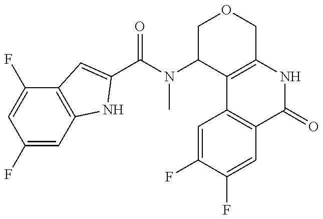 [SMILES: CN(C(=O)c1cc2c(F)cc(F)cc2[nH]1)C1COCc2[nH]c(=O)c3cc(F)c(F)cc3c21] | 0.01 |
| | N-(8,9-difluoro-6-oxo-1,4,5,6-tetrahydro-2H-pyrano[3,4-c]isoquinolin-1-yl)- | |
| | 4,6-difluoro-N-methyl-1H-indole-2-carboxamide | |
| | enantiomer II | |
| 45 | 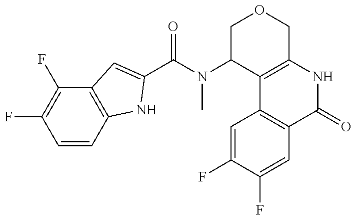 [SMILES: CN(C(=O)c1cc2c(F)c(F)ccc2[nH]1)C1COCc2[nH]c(=O)c3cc(F)c(F)cc3c21] | 0.31 |
| | N-(8,9-difluoro-6-oxo-1,4,5,6-tetrahydro-2H-pyrano[3,4-c]isoquinolin-1-yl)- | |
| | 4,5-difluoro-N-methyl-1H-indole-2-carboxamide | |
| | enantiomer I | |
| 46 | 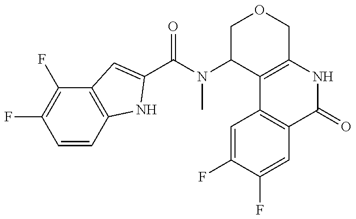 [SMILES: CN(C(=O)c1cc2c(F)c(F)ccc2[nH]1)C1COCc2[nH]c(=O)c3cc(F)c(F)cc3c21] | 0.005 |
| | N-(8,9-difluoro-6-oxo-1,4,5,6-tetrahydro-2H-pyrano[3,4-c]isoquinolin-1-yl)- | |
| | 4,5-difluoro-N-methyl-1H-indole-2-carboxamide | |
| | enantiomer II | |
| 47 | 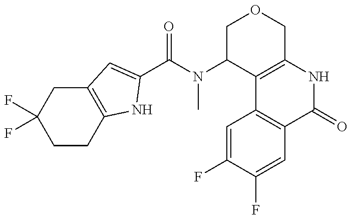 [SMILES: CN(C(=O)c1cc2c([nH]1)CCC(F)(F)C2)C1COCc2[nH]c(=O)c3cc(F)c(F)cc3c21] | 0.02 |
| | N-(8,9-difluoro-6-oxo-1,4,5,6-tetrahydro-2H-pyrano[3,4-c]isoquinolin-1-y1)- | |
| | 5,5-difluoro-N-methyl-4,5,6,7-tetrahydro-1H-indole-2-carboxamide | |
| | enantiomer I | |
| 48 | 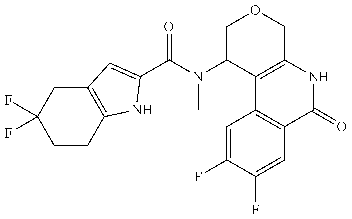 [SMILES: CN(C(=O)c1cc2c([nH]1)CCC(F)(F)C2)C1COCc2[nH]c(=O)c3cc(F)c(F)cc3c21] | 0.77 |
| | N-(8,9-difluoro-6-oxo-1,4,5,6-tetrahydro-2H-pyrano[3,4-c]isoquinolin-1-yl)- | |
| | 5,5-difluoro-N-methyl-4,5,6,7-tetrahydro-1H-indole-2-carboxamide | |
| | enantiomer II | |
| 49 | 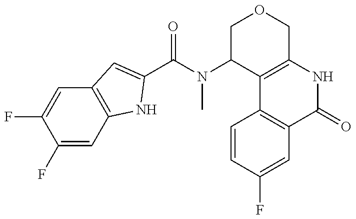 [SMILES: CN(C(=O)c1cc2cc(F)c(F)cc2[nH]1)C1COCc2[nH]c(=O)c3cc(F)ccc3c21] | 2.1 |
| | c]isoquinolin-1-yl)-N-methyl-1H-indole-2-carboxamide | |
| | enantiomer I | |
| 50 | 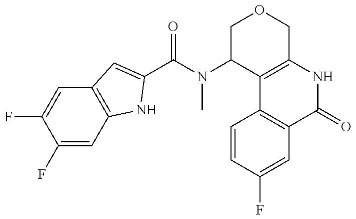 [SMILES: CN(C(=O)c1cc2cc(F)c(F)cc2[nH]1)C1COCc2[nH]c(=O)c3cc(F)ccc3c21] | 0.01 |
| | 5,6-difluoro-N-(8-fluoro-6-oxo-1,4,5,6-tetrahydro-2H-pyrano[3,4- | |
| | c]isoquinolin-1-y1)-N-methyl-1H-indole-2-carboxamide | |
| | enantiomer II | |
| 51 | 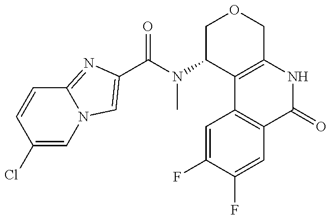 [SMILES: CN(C(=O)c1cn2cc(Cl)ccc2n1)[C@@H]1COCc2[nH]c(=O)c3cc(F)c(F)cc3c21] | 1.3 |
| | (S)-6-Chloro-N-(8,9-difluoro-6-oxo-1,4,5,6-tetrahydro-2H-pyrano[3,4- | |
| | c]isoquinolin-1-yl)-N-methylimidazo[1,2-a]pyridine-2-carboxamide | |
| 52 | 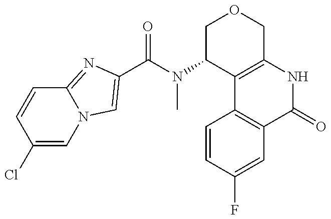 [SMILES: CN(C(=O)c1cn2cc(Cl)ccc2n1)[C@@H]1COCc2[nH]c(=O)c3cc(F)ccc3c21] | 4.1 |
| | (S)-6-Chloro-N-(8-fluoro-6-oxo-1,4,5,6-tetrahydro-2H-pyrano[3,4- | |
| | c]isoquinolin-1-yl)-N-methylimidazo[1,2-a]pyridine-2-carboxamide | |
| 53 | 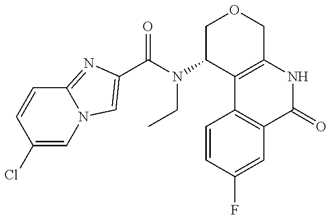 [SMILES: CCN(C(=O)c1cn2cc(Cl)ccc2n1)[C@@H]1COCc2[nH]c(=O)c3cc(F)ccc3c21] | 2.5 |
| | (S)-6-chloro-N-(8-fluoro-6-oxo-1,4,5,6-tetrahydro-2H-pyrano[3,4- | |
| | c]isoquinolin-1-y1)-N-ethylimidazo[1,2-a]pyridine-2-carboxamide | |
| 54 | 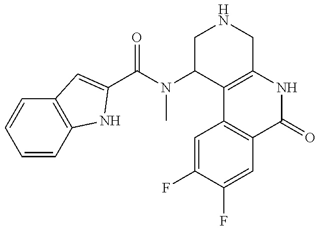 [SMILES: CN(C(=O)c1cc2ccccc2[nH]1)C1CNCc2[nH]c(=O)c3cc(F)c(F)cc3c21] | 0.85 |
| | N-(8,9-difluoro-6-oxo-1,2,3,4,5,6-hexahydrobenzo[c][1,7]naphthyridin-1- | |
| | y1)-N-methyl-1H-indole-2-carboxamide | |
| | enantiomer I | |
| 55 | 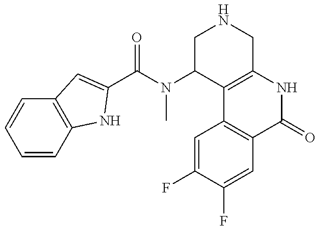 [SMILES: CN(C(=O)c1cc2ccccc2[nH]1)C1CNCc2[nH]c(=O)c3cc(F)c(F)cc3c21] | 0.01 |
| | N-(8,9-difluoro-6-oxo-1,2,3,4,5,6-hexahydrobenzo[c][1,7]naphthyridin-1- | |
| | yl)-N-methyl-1H-indole-2-carboxamide | |
| | enantiomer II | |
| 56 | 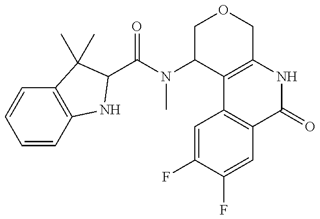 [SMILES: CN(C(=O)C1Nc2ccccc2C1(C)C)C1COCc2[nH]c(=O)c3cc(F)c(F)cc3c21] | 18 |
| | N-(8,9-difluoro-6-oxo-1,4,5,6-tetrahydro-2H-pyrano[3,4-c]isoquinolin-1-yl)- | |
| | N,3,3-trimethylindoline-2-carboxamide | |
| | diastereoisomer IIa | |
| 57 | 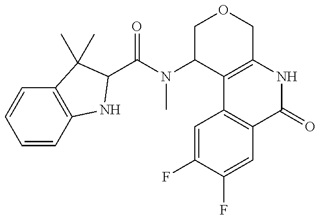 [SMILES: CN(C(=O)C1Nc2ccccc2C1(C)C)C1COCc2[nH]c(=O)c3cc(F)c(F)cc3c21] | 23 |
| | N-(8,9-difluoro-6-oxo-1,4,5,6-tetrahydro-2H-pyrano[3,4-c]isoquinolin-1-yl)- | |
| | N,3,3-trimethylindoline-2-carboxamide | |
| | diastereoisomer Ia | |
| 59 | 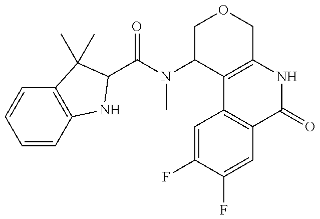 [SMILES: CN(C(=O)C1Nc2ccccc2C1(C)C)C1COCc2[nH]c(=O)c3cc(F)c(F)cc3c21] | 22 |
| | N-(8,9-difluoro-6-oxo-1,4,5,6-tetrahydro-2H-pyrano[3,4-c]isoquinolin-1-yl)- | |
| | N,3,3-trimethylindoline-2-carboxamide | |
| | diastereoisomer IIb | |
| 60 | 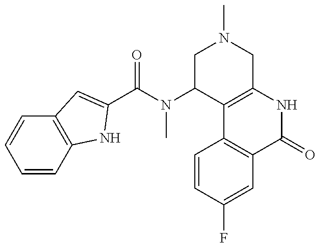 [SMILES: CN1Cc2[nH]c(=O)c3cc(F)ccc3c2C(N(C)C(=O)c2cc3ccccc3[nH]2)C1] | 5.9 |
| | N-(8-fluoro-3-methyl-6-oxo-1,2,3,4,5,6- | |
| | hexahydrobenzo[c][1,7]naphthyridin-1-yl)-N-methyl-1H-indole-2- | |
| | carboxamide | |
| | enantiomer I | |
| 61 | 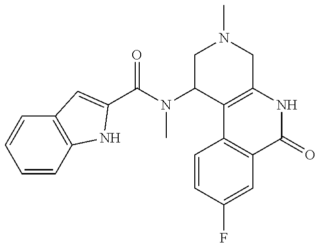 [SMILES: CN1Cc2[nH]c(=O)c3cc(F)ccc3c2C(N(C)C(=O)c2cc3ccccc3[nH]2)C1] | 2.3 |
| | N-(8-fluoro-3-methyl-6-oxo-1,2,3,4,5,6- | |
| | hexahydrobenzo[c][1,7]naphthyridin-1-yl)-N-methyl-1H-indole-2- | |
| | carboxamide | |
| | enantiomer II | |
| 62 | 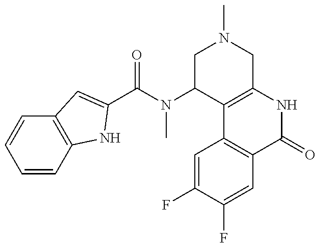 [SMILES: CN1Cc2[nH]c(=O)c3cc(F)c(F)cc3c2C(N(C)C(=O)c2cc3ccccc3[nH]2)C1] | 5.1 |
| | N-(8,9-difluoro-3-methyl-6-oxo-1,2,3,4,5,6- | |
| | hexahydrobenzo[c][1,7]naphthyridin-1-yl)-N-methyl-1H-indole-2- | |
| | carboxamide | |
| | enantiomer I | |
| 63 | 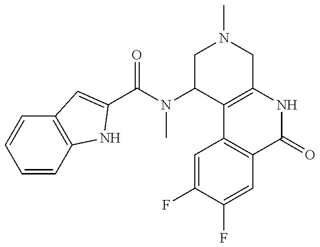 [SMILES: CN1Cc2[nH]c(=O)c3cc(F)c(F)cc3c2C(N(C)C(=O)c2cc3ccccc3[nH]2)C1] | 0.17 |
| | N-(8,9-difluoro-3-methyl-6-oxo-1,2,3,4,5,6- | |
| | hexahydrobenzo[c][1,7]naphthyridin-1-yl)-N-methyl-1H-indole-2- | |
| | carboxamide | |
| | enantiomer II | |
| 64 | 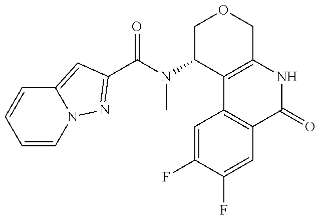 [SMILES: CN(C(=O)c1cc2ccccn2n1)[C@@H]1COCc2[nH]c(=O)c3cc(F)c(F)cc3c21] | 0.63 |
| | (S)-N-(8,9-Difluoro-6-oxo-1,4,5,6-tetrahydro-2H-pyrano[3,4-c]isoquinolin- | |
| | 1-yl)-N-methylpyrazolo[1,5-a]pyridine-2-carboxamide | |
| 65 | 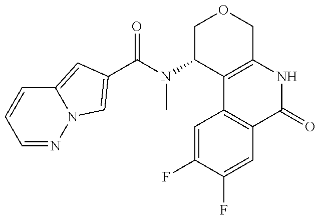 [SMILES: CN(C(=O)c1cc2cccnn2c1)[C@@H]1COCc2[nH]c(=O)c3cc(F)c(F)cc3c21] | 0.15 |
| | (S)-N-(8,9-Difluoro-6-oxo-1,4,5,6-tetrahydro-2H-pyrano[3,4-c]isoquinolin- | |
| | 1-yl)-N-methylpyrrolo[1,2-b]pyridazine-6-carboxamide | |
| 66 | 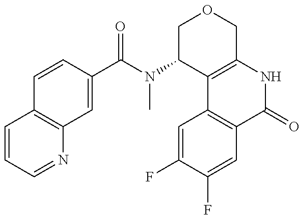 [SMILES: CN(C(=O)c1ccc2cccnc2c1)[C@@H]1COCc2[nH]c(=O)c3cc(F)c(F)cc3c21] | 2.3 |
| | (S)-N-(8,9-Difluoro-6-oxo-1,4,5,6-tetrahydro-2H-pyrano[3,4-c]isoquinolin- | |
| | 1-yl)-N-methylquinoline-7-carboxamide | |
| 67 | 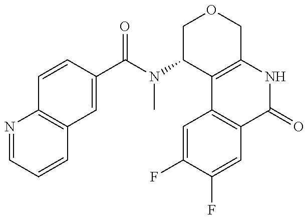 [SMILES: CN(C(=O)c1ccc2ncccc2c1)[C@@H]1COCc2[nH]c(=O)c3cc(F)c(F)cc3c21] | 1.6 |
| | (S)-N-(8,9-Difluoro-6-oxo-1,4,5,6-tetrahydro-2H-pyrano[3,4-c]isoquinolin- | |
| | 1-yl)-N-methylquinoline-6-carboxamide | |
| 68 | 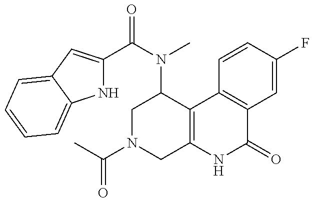 [SMILES: CC(=O)N1Cc2[nH]c(=O)c3cc(F)ccc3c2C(N(C)C(=O)c2cc3ccccc3[nH]2)C1] | 5.5 |
| | N-(3-Acetyl-8-fluoro-6-oxo-1,2,3,4,5,6- | |
| | hexahydrobenzo[c][1,7]naphthyridin-1-yl)-N-methyl-1H-indole-2- | |
| | carboxamide | |
| | enantiomer I | |
| 69 | 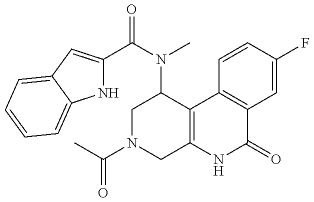 [SMILES: CC(=O)N1Cc2[nH]c(=O)c3cc(F)ccc3c2C(N(C)C(=O)c2cc3ccccc3[nH]2)C1] | 21 |
| | N-(3-Acetyl-8-fluoro-6-oxo-1,2,3,4,5,6- | |
| | hexahydrobenzo[c][1,7]naphthyridin-1-yl)-N-methyl-1H-indole-2- | |
| | carboxamide | |
| | enantiomer II | |
| 70 | 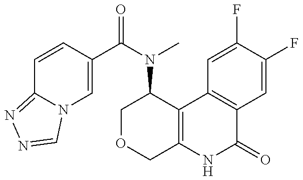 [SMILES: CN(C(=O)c1ccc2nncn2c1)[C@@H]1COCc2[nH]c(=O)c3cc(F)c(F)cc3c21] | 22 |
| | (S)-N-(8,9-Difluoro-6-oxo-1,4,5,6-tetrahydro-2H-pyrano[3,4- | |
| | c]isoquinolin-1-y1)-N-methyl-[1,2,4]triazolo[4,3-a]pyridine-6- | |
| | carboxamide | |
| 71 | 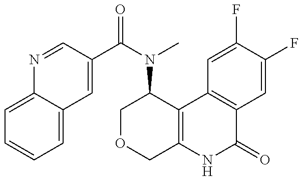 [SMILES: CN(C(=O)c1cnc2ccccc2c1)[C@@H]1COCc2[nH]c(=O)c3cc(F)c(F)cc3c21] | 0.33 |
| | (S)-N-(8,9-Difluoro-6-oxo-1,4,5,6-tetrahydro-2H-pyrano[3,4- | |
| | c]isoquinolin-1-y1)-N-methylquinoline-3-carboxamide | |
| 72 | 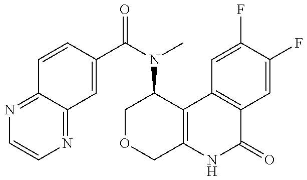 [SMILES: CN(C(=O)c1ccc2nccnc2c1)[C@@H]1COCc2[nH]c(=O)c3cc(F)c(F)cc3c21] | 3.8 |
| | (S)-N-(8,9-Difluoro-6-oxo-1,4,5,6-tetrahydro-2H-pyrano[3,4- | |
| | c]isoquinolin-1-y1)-N-methylquinoxaline-6-carboxamide | |
| 73 | 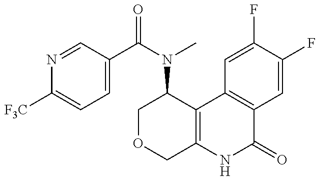 [SMILES: CN(C(=O)c1ccc(C(F)(F)F)nc1)[C@@H]1COCc2[nH]c(=O)c3cc(F)c(F)cc3c21] | 0.12 |
| | (S)-N-(8,9-Difluoro-6-oxo-1,4,5,6-tetrahydro-2H-pyrano[3,4- | |
| | c]isoquinolin-1-y1)-N-methyl-6-(trifluoromethyl)nicotinamide | |
| 74 | 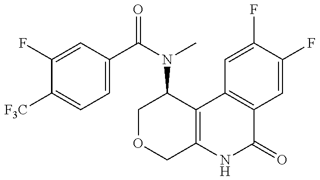 [SMILES: CN(C(=O)c1ccc(C(F)(F)F)c(F)c1)[C@@H]1COCc2[nH]c(=O)c3cc(F)c(F)cc3c21] | 0.01 |
| | (S)-N-(8,9-Difluoro-6-oxo-1,4,5,6-tetrahydro-2H-pyrano[3,4- | |
| | c]isoquinolin-1-yl)-3-fluoro-N-methyl-4-(trifluoromethyl)benzamide | |
| 75 | 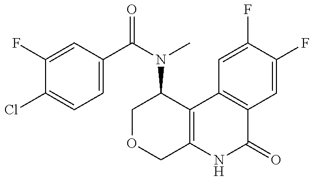 [SMILES: CN(C(=O)c1ccc(Cl)c(F)c1)[C@@H]1COCc2[nH]c(=O)c3cc(F)c(F)cc3c21] | 0.02 |
| | (S)-4-Chloro-N-(8,9-difluoro-6-oxo-1,4,5,6-tetrahydro-2H- | |
| | pyrano[3,4-c]isoquinolin-1-y1)-3-fluoro-N-methylbenzamide | |
| 76 | 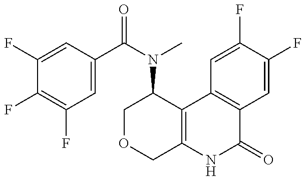 [SMILES: CN(C(=O)c1cc(F)c(F)c(F)c1)[C@@H]1COCc2[nH]c(=O)c3cc(F)c(F)cc3c21] | 0.09 |
| | (S)-N-(8,9-Difluoro-6-oxo-1,4,5,6-tetrahydro-2H-pyrano[3,4- | |
| | c]isoquinolin-1-yl)-3,4,5-trifluoro-N-methylbenzamide | |
| 77 | 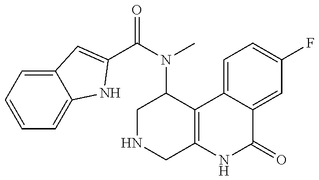 [SMILES: CN(C(=O)c1cc2ccccc2[nH]1)C1CNCc2[nH]c(=O)c3cc(F)ccc3c21] | 0.15 |
| | N-(8-Fluoro-6-oxo-1,2,3,4,5,6-hexahydrobenzo[c][1,7]naphthyridin- | |
| | 1-y1)-N-methyl-1H-indole-2-carboxamide | |
| | enantiomer I | |
| 78 | 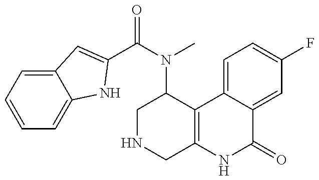 [SMILES: CN(C(=O)c1cc2ccccc2[nH]1)C1CNCc2[nH]c(=O)c3cc(F)ccc3c21] | 8.1 |
| | N-(8-Fluoro-6-oxo-1,2,3,4,5,6-hexahydrobenzo[c][1,7]naphthyridin- | |
| | 1-yl)-N-methyl-1H-indole-2-carboxamide | |
| | enantiomer II | |
| 79 | 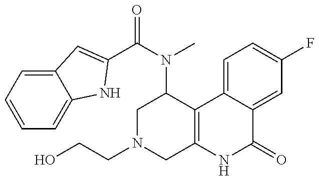 [SMILES: CN(C(=O)c1cc2ccccc2[nH]1)C1CN(CCO)Cc2[nH]c(=O)c3cc(F)ccc3c21] | 10 |
| | N-(8-Fluoro-3-(2-hydroxyethyl)-6-oxo-1,2,3,4,5,6- | |
| | hexahydrobenzo[c][1,7]naphthyridin-1-yl)-N-methyl-1H-indole-2- | |
| | carboxamide | |
| | enantiomer I | |
| 80 | 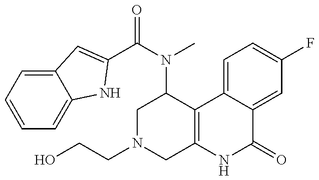 [SMILES: CN(C(=O)c1cc2ccccc2[nH]1)C1CN(CCO)Cc2[nH]c(=O)c3cc(F)ccc3c21] | 6.3 |
| | N-(8-Fluoro-3-(2-hydroxyethyl)-6-oxo-1,2,3,4,5,6- | |
| | hexahydrobenzo[c][1,7]naphthyridin-1-y1)-N-methyl-1H-indole-2- | |
| | carboxamide | |
| | enantiomer II | |
| 81 | 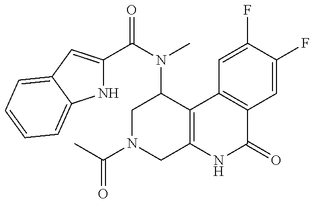 [SMILES: CC(=O)N1Cc2[nH]c(=O)c3cc(F)c(F)cc3c2C(N(C)C(=O)c2cc3ccccc3[nH]2)C1] | 14 |
| | N-(3-Acetyl-8,9-difluoro-6-oxo-1,2,3,4,5,6- | |
| | hexahydrobenzo[c][1,7]naphthyridin-1-yl)-N-methyl-1H-indole-2- | |
| | carboxamide | |
| | enantiomer I | |
| 82 | 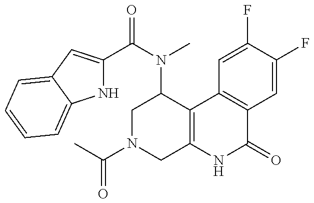 [SMILES: CC(=O)N1Cc2[nH]c(=O)c3cc(F)c(F)cc3c2C(N(C)C(=O)c2cc3ccccc3[nH]2)C1] | 3.5 |
| | N-(3-Acetyl-8,9-difluoro-6-oxo-1,2,3,4,5,6- | |
| | hexahydrobenzo[c][1,7]naphthyridin-1-yl)-N-methyl-1H-indole-2- | |
| | carboxamide | |
| | enantiomer II | |
| 83 | 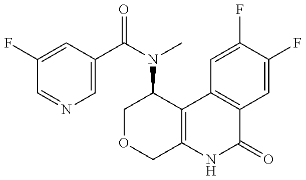 [SMILES: CN(C(=O)c1cncc(F)c1)[C@@H]1COCc2[nH]c(=O)c3cc(F)c(F)cc3c21] | 7.0 |
| | (S)-N-(8,9-Difluoro-6-oxo-1,4,5,6-tetrahydro-2H-pyrano[3,4- | |
| | c]isoquinolin-1-y1)-5-fluoro-N-methylnicotinamide | |
| 84 | 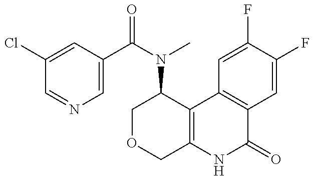 [SMILES: CN(C(=O)c1cncc(Cl)c1)[C@@H]1COCc2[nH]c(=O)c3cc(F)c(F)cc3c21] | 5.7 |
| | (S)-5-Chloro-N-(8,9-difluoro-6-oxo-1,4,5,6-tetrahydro-2H- | |
| | pyrano[3,4-c]isoquinolin-1-yl)-N-methylnicotinamide | |
| 85 | 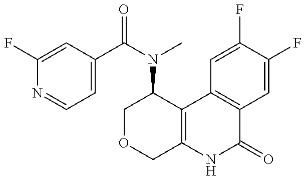 [SMILES: CN(C(=O)c1ccnc(F)c1)[C@@H]1COCc2[nH]c(=O)c3cc(F)c(F)cc3c21] | 0.63 |
| | (S)-N-(8,9-Difluoro-6-oxo-1,4,5,6-tetrahydro-2H-pyrano[3,4- | |
| | c]isoquinolin-1-y1)-2-fluoro-N-methylisonicotinamide | |
| 86 | 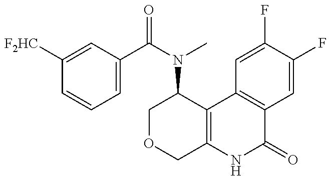 [SMILES: CN(C(=O)c1cccc(C(F)F)c1)[C@@H]1COCc2[nH]c(=O)c3cc(F)c(F)cc3c21] | 0.03 |
| | (S)-N-(8,9-Difluoro-6-oxo-1,4,5,6-tetrahydro-2H-pyrano[3,4- | |
| | c]isoquinolin-1-yl)-3-(difluoromethyl)-N-methylbenzamide | |
| 87 | 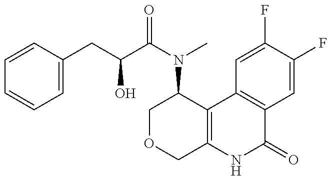 [SMILES: CN(C(=O)[C@@H](O)Cc1ccccc1)[C@@H]1COCc2[nH]c(=O)c3cc(F)c(F)cc3c21] | 3.2 |
| | (S)-N-((S)-8,9-Difluoro-6-oxo-1,4,5,6-tetrahydro-2H-pyrano[3,4- | |
| | c]isoquinolin-1-y1)-2-hydroxy-N-methyl-3-phenylpropanamide | |
| 88 | 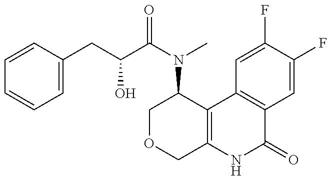 [SMILES: CN(C(=O)[C@H](O)Cc1ccccc1)[C@@H]1COCc2[nH]c(=O)c3cc(F)c(F)cc3c21] | 0.48 |
| | (R)-N-((S)-8,9-Difluoro-6-oxo-1,4,5,6-tetrahydro-2H-pyrano[3,4- | |
| | c]isoquinolin-1-yl)-2-hydroxy-N-methyl-3-phenylpropanamide | |
| 89 | 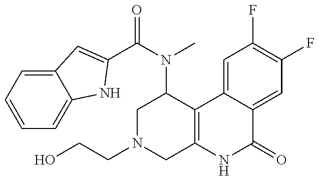 [SMILES: CN(C(=O)c1cc2ccccc2[nH]1)C1CN(CCO)Cc2[nH]c(=O)c3cc(F)c(F)cc3c21] | 7.9 |
| | N-(8,9-Difluoro-3-(2-hydroxyethyl)-6-oxo-1,2,3,4,5,6- | |
| | hexahydrobenzo[c][1,7]naphthyridin-1-y1)-N-methyl-1H-indole-2- | |
| | carboxamide | |
| | enantiomer I | |
| 90 | 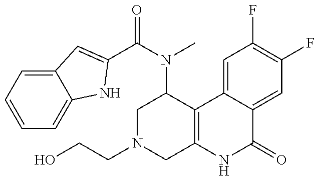 [SMILES: CN(C(=O)c1cc2ccccc2[nH]1)C1CN(CCO)Cc2[nH]c(=O)c3cc(F)c(F)cc3c21] | 0.5 |
| | N-(8,9-Difluoro-3-(2-hydroxyethyl)-6-oxo-1,2,3,4,5,6- | |
| | hexahydrobenzo[c][1,7]naphthyridin-1-yl)-N-methyl-1H-indole-2- | |
| | carboxamide | |
| | enantiomer II | |
| 91 | 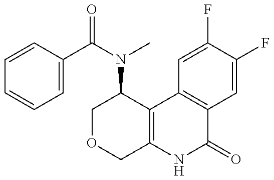 [SMILES: CN(C(=O)c1ccccc1)[C@@H]1COCc2[nH]c(=O)c3cc(F)c(F)cc3c21] | 1.01 |
| | (S)-N-(8,9-Difluoro-6-oxo-1,4,5,6-tetrahydro-2H-pyrano[3,4- | |
| | c]isoquinolin-1-y1)-N-methylbenzamide | |
| 92 | 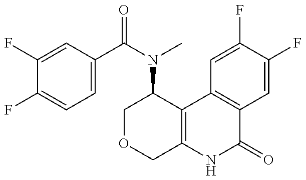 [SMILES: CN(C(=O)c1ccc(F)c(F)c1)[C@@H]1COCc2[nH]c(=O)c3cc(F)c(F)cc3c21] | 0.28 |
| | (S)-N-(8,9-Difluoro-6-oxo-1,4,5,6-tetrahydro-2H-pyrano[3,4- | |
| | c]isoquinolin-1-yl)-3,4-difluoro-N-methylbenzamide | |
| 93 | 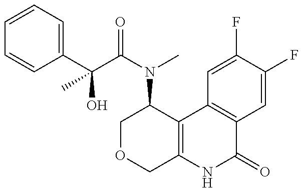 [SMILES: CN(C(=O)[C@@](C)(O)c1ccccc1)[C@@H]1COCc2[nH]c(=O)c3cc(F)c(F)cc3c21] | 12 |
| | (S)-N-((S)-8,9-Difluoro-6-oxo-1,4,5,6-tetrahydro-2H-pyrano[3,4- | |
| | c]isoquinolin-1-y1)-2-hydroxy-N-methyl-2-phenylpropanamide | |
| 94 | 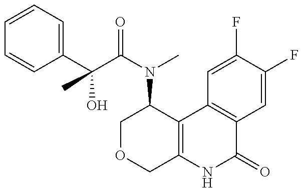 [SMILES: CN(C(=O)[C@](C)(O)c1ccccc1)[C@@H]1COCc2[nH]c(=O)c3cc(F)c(F)cc3c21] | 3.2 |
| | (R)-N-((S)-8,9-Difluoro-6-oxo-1,4,5,6-tetrahydro-2H-pyrano[3,4- | |
| | c]isoquinolin-1-y1)-2-hydroxy-N-methyl-2-phenylpropanamide | |
| 95 | 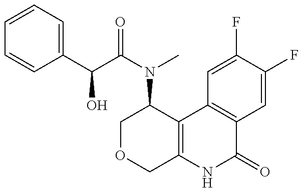 [SMILES: CN(C(=O)[C@@H](O)c1ccccc1)[C@@H]1COCc2[nH]c(=O)c3cc(F)c(F)cc3c21] | 24 |
| | (S)-N-((S)-8,9-Difluoro-6-oxo-1,4,5,6-tetrahydro-2H-pyrano[3,4- | |
| | c]isoquinolin-1-y1)-2-hydroxy-N-methyl-2-phenylacetamide | |
| 96 | 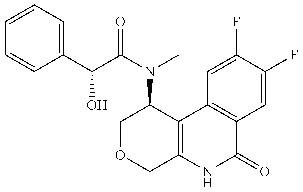 [SMILES: CN(C(=O)[C@H](O)c1ccccc1)[C@@H]1COCc2[nH]c(=O)c3cc(F)c(F)cc3c21] | 10 |
| | (R)-N-((S)-8,9-Difluoro-6-oxo-1,4,5,6-tetrahydro-2H-pyrano[3,4- | |
| | c]isoquinolin-1-yl)-2-hydroxy-N-methyl-2-phenylacetamide | |
| 97 | 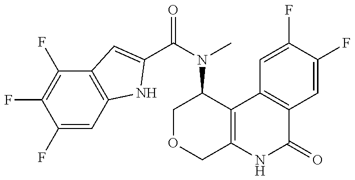 [SMILES: CN(C(=O)c1cc2c(F)c(F)c(F)cc2[nH]1)[C@@H]1COCc2[nH]c(=O)c3cc(F)c(F)cc3c21] | 0.01 |
| | (S)-N-(8,9-Difluoro-6-oxo-1,4,5,6-tetrahydro-2H-pyrano[3,4- | |
| | c]isoquinolin-1-y1)-4,5,6-trifluoro-N-methyl-1H-indole-2- | |
| | carboxamide | |
| 98 | 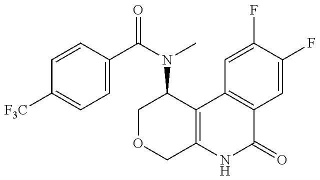 [SMILES: CN(C(=O)c1ccc(C(F)(F)F)cc1)[C@@H]1COCc2[nH]c(=O)c3cc(F)c(F)cc3c21] | 0.01 |
| | (S)-N-(8,9-Difluoro-6-oxo-1,4,5,6-tetrahydro-2H-pyrano[3,4- | |
| | c]isoquinolin-1-y1)-N-methyl-4-(trifluoromethyl)benzamide | |
| 99 | 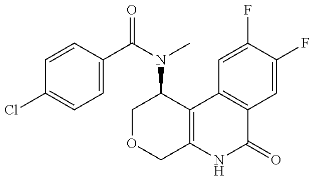 [SMILES: CN(C(=O)c1ccc(Cl)cc1)[C@@H]1COCc2[nH]c(=O)c3cc(F)c(F)cc3c21] | 0.02 |
| | (S)-4-Chloro-N-(8,9-difluoro-6-oxo-1,4,5,6-tetrahydro-2H- | |
| | pyrano[3,4-c]isoquinolin-1-yl)-N-methylbenzamide | |
| 100 | 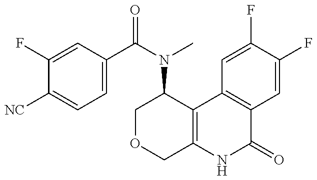 [SMILES: CN(C(=O)c1ccc(C#N)c(F)c1)[C@@H]1COCc2[nH]c(=O)c3cc(F)c(F)cc3c21] | 0.04 |
| | (S)-4-Cyano-N-(8,9-difluoro-6-oxo-1,4,5,6-tetrahydro-2H-pyrano[3,4- | |
| | c]isoquinolin-1-yl)-3-fluoro-N-methylbenzamide | |
| 101 | 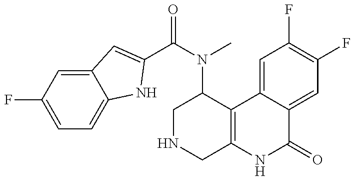 [SMILES: CN(C(=O)c1cc2cc(F)ccc2[nH]1)C1CNCc2[nH]c(=O)c3cc(F)c(F)cc3c21] | 0.01 |
| | N-(8,9-Difluoro-6-oxo-1,2,3,4,5,6- | |
| | hexahydrobenzo[c][1,7]naphthyridin-1-y1)-5-fluoro-N-methyl-1H- | |
| | indole-2-carboxamide | |
| | enantiomer I | |
| 102 | 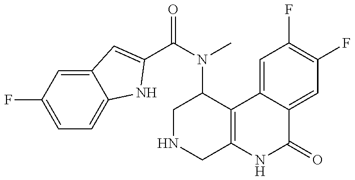 [SMILES: CN(C(=O)c1cc2cc(F)ccc2[nH]1)C1CNCc2[nH]c(=O)c3cc(F)c(F)cc3c21] | 1.8 |
| | N-(8,9-Difluoro-6-oxo-1,2,3,4,5,6- | |
| | hexahydrobenzo[c][1,7]naphthyridin-1-yl)-5-fluoro-N-methyl-1H- | |
| | indole-2-carboxamide | |
| | enantiomer II | |
| 103 | 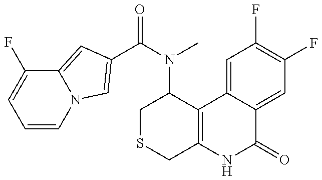 [SMILES: CN(C(=O)c1cc2c(F)cccn2c1)C1CSCc2[nH]c(=O)c3cc(F)c(F)cc3c21] | 2.5 |
| | N-(8,9-Difluoro-6-oxo-1,4,5,6-tetrahydro-2H-thiopyrano[3,4- | |
| | c]isoquinolin-1-y1)-8-fluoro-N-methylindolizine-2-carboxamide | |
| | enantiomer I | |
| 104 | 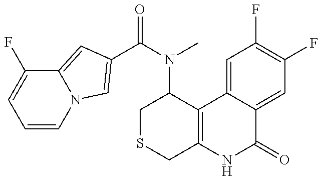 [SMILES: CN(C(=O)c1cc2c(F)cccn2c1)C1CSCc2[nH]c(=O)c3cc(F)c(F)cc3c21] | 0.01 |
| | N-(8,9-Difluoro-6-oxo-1,4,5,6-tetrahydro-2H-thiopyrano[3,4- | |
| | c]isoquinolin-1-yl)-8-fluoro-N-methylindolizine-2-carboxamide | |
| | enantiomer II | |
| 105 | 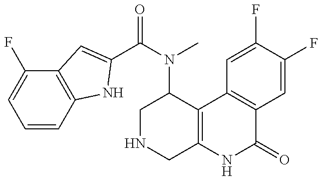 [SMILES: CN(C(=O)c1cc2c(F)cccc2[nH]1)C1CNCc2[nH]c(=O)c3cc(F)c(F)cc3c21] | 0.01 |
| | N-(8,9-Difluoro-6-oxo-1,2,3,4,5,6- | |
| | hexahydrobenzo[c][1,7]naphthyridin-1-y1)-4-fluoro-N-methyl-1H- | |
| | indole-2-carboxamide | |
| | enantiomer I | |
| 106 | 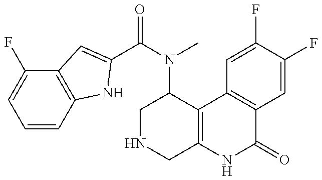 [SMILES: CN(C(=O)c1cc2c(F)cccc2[nH]1)C1CNCc2[nH]c(=O)c3cc(F)c(F)cc3c21] | 0.10 |
| | N-(8,9-Difluoro-6-oxo-1,2,3,4,5,6- | |
| | hexahydrobenzo[c][1,7]naphthyridin-1-yl)-4-fluoro-N-methyl-1H- | |
| | indole-2-carboxamide | |
| | enantiomer II | |
| 107 | 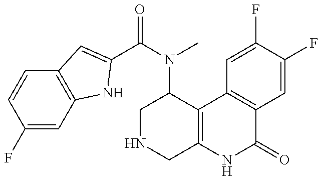 [SMILES: CN(C(=O)c1cc2ccc(F)cc2[nH]1)C1CNCc2[nH]c(=O)c3cc(F)c(F)cc3c21] | 0.02 |
| | N-(8,9-Difluoro-6-oxo-1,2,3,4,5,6- | |
| | hexahydrobenzo[c][1,7]naphthyridin-1-y1)-6-fluoro-N-methyl-1H- | |
| | indole-2-carboxamide | |
| | enantiomer I | |
| 108 | 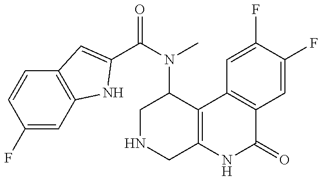 [SMILES: CN(C(=O)c1cc2ccc(F)cc2[nH]1)C1CNCc2[nH]c(=O)c3cc(F)c(F)cc3c21] | 1.6 |
| | N-(8,9-Difluoro-6-oxo-1,2,3,4,5,6- | |
| | hexahydrobenzo[c][1,7]naphthyridin-1-yl)-6-fluoro-N-methyl-1H- | |
| | indole-2-carboxamide | |
| | enantiomer II | |
| 109 | 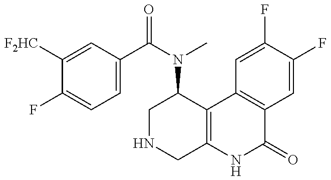 [SMILES: CN(C(=O)c1ccc(F)c(C(F)F)c1)[C@@H]1CNCc2[nH]c(=O)c3cc(F)c(F)cc3c21] | 0.02 |
| | (S)-N-(8,9-Difluoro-6-oxo-1,4,5,6-tetrahydro-2H-pyrano[3,4- | |
| | c]isoquinolin-1-yl)-3-(difluoromethyl)-4-fluoro-N-methylbenzamide | |
| 110 | 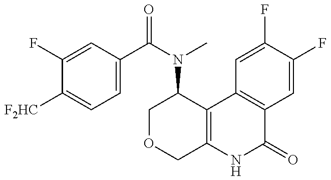 [SMILES: CN(C(=O)c1ccc(C(F)F)c(F)c1)[C@@H]1COCc2[nH]c(=O)c3cc(F)c(F)cc3c21] | 0.01 |
| | (S)-N-(8,9-Difluoro-6-oxo-1,4,5,6-tetrahydro-2H-pyrano[3,4- | |
| | c]isoquinolin-1-yl)-4-(difluoromethyl)-3-fluoro-N-methylbenzamide | |
| 111 | 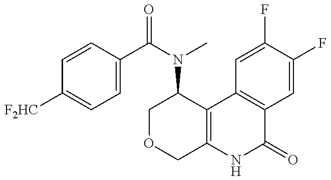 [SMILES: CN(C(=O)c1ccc(C(F)F)cc1)[C@@H]1COCc2[nH]c(=O)c3cc(F)c(F)cc3c21] | 0.02 |
| | (S)-N-(8,9-Difluoro-6-oxo-1,4,5,6-tetrahydro-2H-pyrano[3,4- | |
| | c]isoquinolin-1-y1)-4-(difluoromethyl)-N-methylbenzamide | |
| 112 | 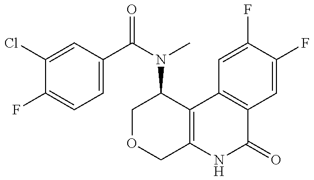 [SMILES: CN(C(=O)c1ccc(F)c(Cl)c1)[C@@H]1COCc2[nH]c(=O)c3cc(F)c(F)cc3c21] | 0.04 |
| | (S)-3-Chloro-N-(8,9-difluoro-6-oxo-1,4,5,6-tetrahydro-2H- | |
| | pyrano[3,4-c]isoquinolin-1-y1)-4-fluoro-N-methylbenzamide | |
| 113 | 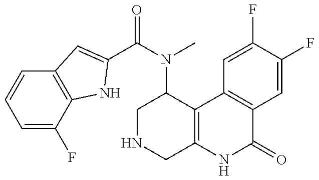 [SMILES: CN(C(=O)c1cc2cccc(F)c2[nH]1)C1CNCc2[nH]c(=O)c3cc(F)c(F)cc3c21] | 0.43 |
| | N-(8,9-Difluoro-6-oxo-1,2,3,4,5,6- | |
| | hexahydrobenzo[c][1,7]naphthyridin-1-yl)-7-fluoro-N-methyl-1H- | |
| | indole-2-carboxamide | |
| | enantiomer I | |
| 114 | 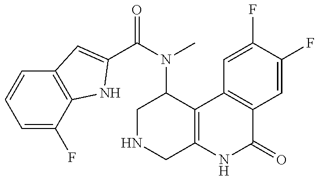 [SMILES: CN(C(=O)c1cc2cccc(F)c2[nH]1)C1CNCc2[nH]c(=O)c3cc(F)c(F)cc3c21] | 0.02 |
| | N-(8,9-Difluoro-6-oxo-1,2,3,4,5,6- | |
| | hexahydrobenzo[c][1,7]naphthyridin-1-y1)-7-fluoro-N-methyl-1H- | |
| | indole-2-carboxamide | |
| | enantiomer II | |
| 115 | 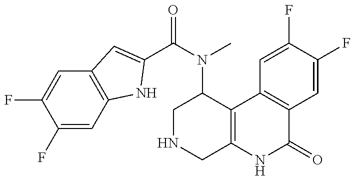 [SMILES: CN(C(=O)c1cc2cc(F)c(F)cc2[nH]1)C1CNCc2[nH]c(=O)c3cc(F)c(F)cc3c21] | 0.02 |
| | N-(8,9-Difluoro-6-oxo-1,2,3,4,5,6- | |
| | hexahydrobenzo[c][1,7]naphthyridin-1-yl)-5,6-difluoro-N-methyl-1H- | |
| | indole-2-carboxamide | |
| | enantiomer I | |
| 116 | 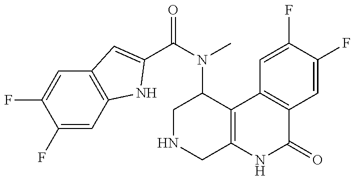 [SMILES: CN(C(=O)c1cc2cc(F)c(F)cc2[nH]1)C1CNCc2[nH]c(=O)c3cc(F)c(F)cc3c21] | 1.3 |
| | N-(8,9-Difluoro-6-oxo-1,2,3,4,5,6- | |
| | hexahydrobenzo[c][1,7]naphthyridin-1-yl)-5,6-difluoro-N-methyl-1H- | |
| | indole-2-carboxamide | |
| | enantiomer II | |
| 117 | 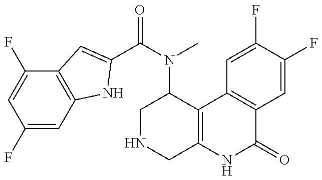 [SMILES: CN(C(=O)c1cc2c(F)cc(F)cc2[nH]1)C1CNCc2[nH]c(=O)c3cc(F)c(F)cc3c21] | 0.02 |
| | N-(8,9-Difluoro-6-oxo-1,2,3,4,5,6- | |
| | hexahydrobenzo[c][1,7]naphthyridin-1-yl)-4,6-difluoro-N-methyl-1H- | |
| | indole-2-carboxamide | |
| | enantiomer I | |
| 118 | 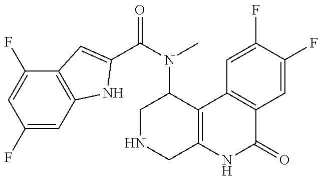 [SMILES: CN(C(=O)c1cc2c(F)cc(F)cc2[nH]1)C1CNCc2[nH]c(=O)c3cc(F)c(F)cc3c21] | 1.2 |
| | N-(8,9-Difluoro-6-oxo-1,2,3,4,5,6- | |
| | hexahydrobenzo[c][1,7]naphthyridin-1-yl)-4,6-difluoro-N-methyl-1H- | |
| | indole-2-carboxamide | |
| | enantiomer II | |
| 119 | 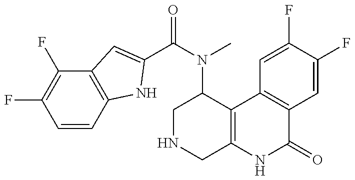 [SMILES: CN(C(=O)c1cc2c(F)c(F)ccc2[nH]1)C1CNCc2[nH]c(=O)c3cc(F)c(F)cc3c21] | 0.01 |
| | N-(8,9-Difluoro-6-oxo-1,2,3,4,5,6- | |
| | hexahydrobenzo[c][1,7]naphthyridin-1-yl)-4,5-difluoro-N-methyl-1H- | |
| | indole-2-carboxamide | |
| | enantiomer I | |
| 120 | 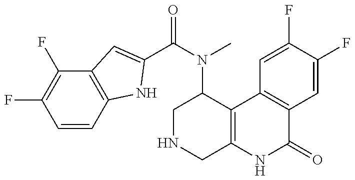 [SMILES: CN(C(=O)c1cc2c(F)c(F)ccc2[nH]1)C1CNCc2[nH]c(=O)c3cc(F)c(F)cc3c21] | 0.85 |
| | N-(8,9-Difluoro-6-oxo-1,2,3,4,5,6- | |
| | hexahydrobenzo[c][1,7]naphthyridin-1-yl)-4,5-difluoro-N-methyl-1H- | |
| | indole-2-carboxamide | |
| | enantiomer II | |
| 121 | 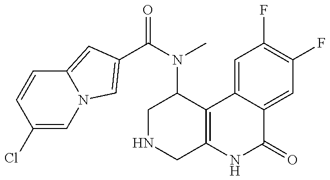 [SMILES: CN(C(=O)c1cc2ccc(Cl)cn2c1)C1CNCc2[nH]c(=O)c3cc(F)c(F)cc3c21] | 0.03 |
| | 6-Chloro-N-(8,9-difluoro-6-oxo-1,2,3,4,5,6- | |
| | hexahydrobenzo[c][1,7]naphthyridin-1-yl)-N-methylindolizine-2- | |
| | carboxamide | |
| | enantiomer I | |
| 122 | 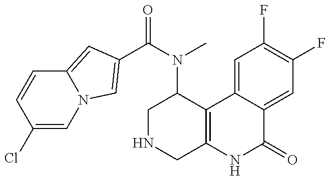 [SMILES: CN(C(=O)c1cc2ccc(Cl)cn2c1)C1CNCc2[nH]c(=O)c3cc(F)c(F)cc3c21] | 1.2 |
| | 6-Chloro-N-(8,9-difluoro-6-oxo-1,2,3,4,5,6- | |
| | hexahydrobenzo[c][1,7]naphthyridin-1-yl)-N-methylindolizine-2- | |
| | carboxamide | |
| | enantiomer II | |
| 123 | 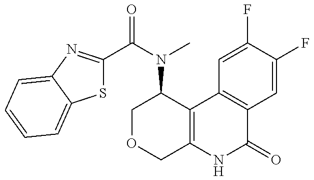 [SMILES: CN(C(=O)c1nc2ccccc2s1)[C@@H]1COCc2[nH]c(=O)c3cc(F)c(F)cc3c21] | 1.5 |
| | (S)-N-(8,9-Difluoro-6-oxo-1,4,5,6-tetrahydro-2H-pyrano[3,4- | |
| | c]isoquinolin-1-y1)-N-methylbenzo[d]thiazole-2-carboxamide | |
| 124 | 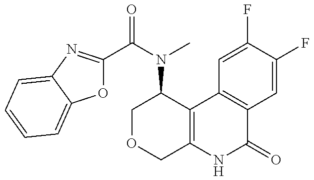 [SMILES: CN(C(=O)c1nc2ccccc2o1)[C@@H]1COCc2[nH]c(=O)c3cc(F)c(F)cc3c21] | 1.1 |
| | (S)-N-(8,9-Difluoro-6-oxo-1,4,5,6-tetrahydro-2H-pyrano[3,4- | |
| | c]isoquinolin-1-yl)-N-methylbenzo[d]oxazole-2-carboxamide | |
| 125 | 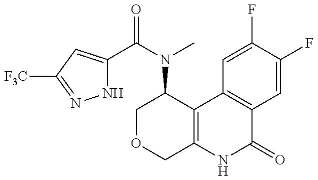 [SMILES: CN(C(=O)c1cc(C(F)(F)F)n[nH]1)[C@@H]1COCc2[nH]c(=O)c3cc(F)c(F)cc3c21] | 0.15 |
| | (S)-N-(8,9-Difluoro-6-oxo-1,4,5,6-tetrahydro-2H-pyrano[3,4- | |
| | c]isoquinolin-1-yl)-N-methyl-3-(trifluoromethyl)-1H-pyrazole-5- | |
| | carboxamide | |
| 126 | 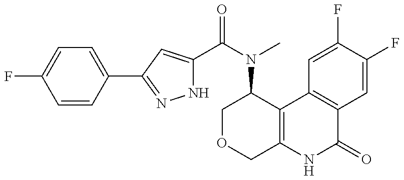 [SMILES: CN(C(=O)c1cc(-c2ccc(F)cc2)n[nH]1)[C@@H]1COCc2[nH]c(=O)c3cc(F)c(F)cc3c21] | 21 |
| | (S)-N-(8,9-Difluoro-6-oxo-1,4,5,6-tetrahydro-2H-pyrano[3,4- | |
| | c]isoquinolin-1-yl)-3-(4-fluorophenyl)-N-methyl-1H-pyrazole-5- | |
| | carboxamide | |
| 127 | 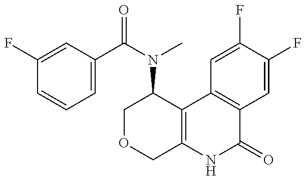 [SMILES: CN(C(=O)c1cccc(F)c1)[C@@H]1COCc2[nH]c(=O)c3cc(F)c(F)cc3c21] | 0.38 |
| | (S)-N-(8,9-Difluoro-6-oxo-1,4,5,6-tetrahydro-2H-pyrano[3,4- | |
| | c]isoquinolin-1-yl)-3-fluoro-N-methylbenzamide | |
| 128 | 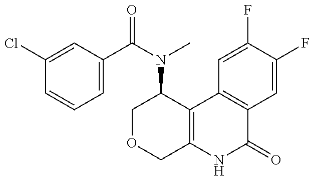 [SMILES: CN(C(=O)c1cccc(Cl)c1)[C@@H]1COCc2[nH]c(=O)c3cc(F)c(F)cc3c21] | 0.09 |
| | (S)-3-Chloro-N-(8,9-difluoro-6-oxo-1,4,5,6-tetrahydro-2H- | |
| | pyrano[3,4-c]isoquinolin-1-y1)-N-methylbenzamide | |
| 129 | 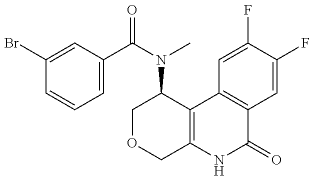 [SMILES: CN(C(=O)c1cccc(Br)c1)[C@@H]1COCc2[nH]c(=O)c3cc(F)c(F)cc3c21] | 0.09 |
| | (S)-3-Bromo-N-(8,9-difluoro-6-oxo-1,4,5,6-tetrahydro-2H- | |
| | pyrano[3,4-c]isoquinolin-1-yl)-N-methylbenzamide | |
| 130 | 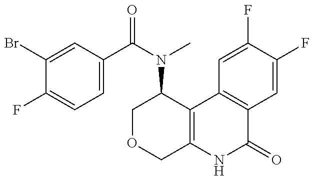 [SMILES: CN(C(=O)c1ccc(F)c(Br)c1)[C@@H]1COCc2[nH]c(=O)c3cc(F)c(F)cc3c21] | 0.01 |
| | (S)-3-Bromo-N-(8,9-difluoro-6-oxo-1,4,5,6-tetrahydro-2H- | |
| | pyrano[3,4-c]isoquinolin-1-yl)-4-fluoro-N-methylbenzamide | |
| 131 | 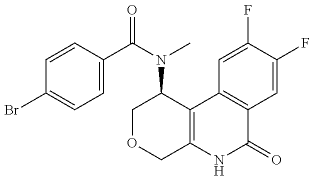 [SMILES: CN(C(=O)c1ccc(Br)cc1)[C@@H]1COCc2[nH]c(=O)c3cc(F)c(F)cc3c21] | 0.01 |
| | (S)-4-Bromo-N-(8,9-difluoro-6-oxo-1,4,5,6-tetrahydro-2H- | |
| | pyrano[3,4-c]isoquinolin-1-yl)-N-methylbenzamide | |
| 132 | 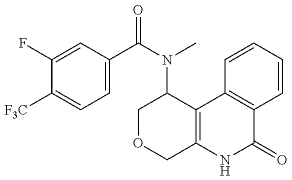 [SMILES: CN(C(=O)c1ccc(C(F)(F)F)c(F)c1)C1COCc2[nH]c(=O)c3ccccc3c21] | 0.06 |
| | 3-Fluoro-N-methyl-N-(6-oxo-1,4,5,6-tetrahydro-2H-pyrano[3,4- | |
| | c]isoquinolin-1-yl)-4-(trifluoromethyl)benzamide | |
| 133 | 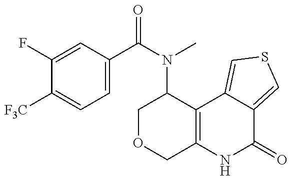 [SMILES: CN(C(=O)c1ccc(C(F)(F)F)c(F)c1)C1COCc2[nH]c(=O)c3cscc3c21] | 0.06 |
| | 3-Fluoro-N-methyl-N-(4-oxo-4,5,8,9-tetrahydro-6H-pyrano[3,4- | |
| | b]thieno[3,4-d]pyridin-9-yl)-4-(trifluoromethyl)benzamide | |
| 134 | 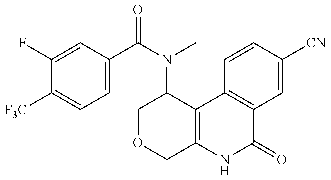 [SMILES: CN(C(=O)c1ccc(C(F)(F)F)c(F)c1)C1COCc2[nH]c(=O)c3cc(C#N)ccc3c21] | 2.8 |
| | N-(8-Cyano-6-oxo-1,4,5,6-tetrahydro-2H-pyrano[3,4-c]isoquinolin-1- | |
| | yl)-3-fluoro-N-methyl-4-(trifluoromethyl)benzamide | |
| 135 | 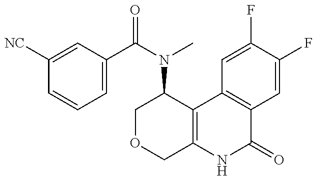 [SMILES: CN(C(=O)c1cccc(C#N)c1)[C@@H]1COCc2[nH]c(=O)c3cc(F)c(F)cc3c21] | 0.77 |
| | (S)-3-Cyano-N-(8,9-difluoro-6-oxo-1,4,5,6-tetrahydro-2H-pyrano[3,4- | |
| | c]isoquinolin-1-y1)-N-methylbenzamide | |
| 136 | 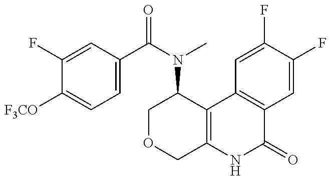 [SMILES: CN(C(=O)c1ccc(OC(F)(F)F)c(F)c1)[C@@H]1COCc2[nH]c(=O)c3cc(F)c(F)cc3c21] | 0.04 |
| | (S)-N-(8,9-Difluoro-6-oxo-1,4,5,6-tetrahydro-2H-pyrano[3,4- | |
| | c]isoquinolin-1-y1)-3-fluoro-N-methyl-4-(trifluoromethoxy)benzamide | |
| 137 | 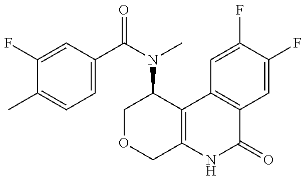 [SMILES: Cc1ccc(C(=O)N(C)[C@@H]2COCc3[nH]c(=O)c4cc(F)c(F)cc4c32)cc1F] | 0.06 |
| | (S)-N-(8,9-Difluoro-6-oxo-1,4,5,6-tetrahydro-2H-pyrano[3,4- | |
| | c]isoquinolin-1-y1)-3-fluoro-N,4-dimethylbenzamide | |
| 138 | 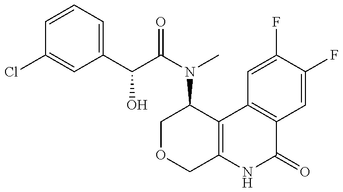 [SMILES: CN(C(=O)[C@H](O)c1cccc(Cl)c1)[C@@H]1COCc2[nH]c(=O)c3cc(F)c(F)cc3c21] | 1.2 |
| | (R)-2-(3-Chlorophenyl)-N-((S)-8,9-difluoro-6-oxo-1,4,5,6-tetrahydro- | |
| | 2H-pyrano[3,4-c]isoquinolin-1-y1)-2-hydroxy-N-methylacetamide | |
| 139 | 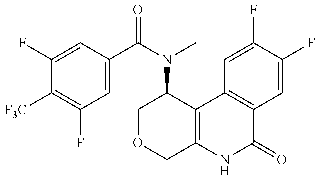 [SMILES: CN(C(=O)c1cc(F)c(C(F)(F)F)c(F)c1)[C@@H]1COCc2[nH]c(=O)c3cc(F)c(F)cc3c21] | 0.01 |
| | (S)-N-(8,9-Difluoro-6-oxo-1,4,5,6-tetrahydro-2H-pyrano[3,4- | |
| | c]isoquinolin-1-yl)-3,5-difluoro-N-methyl-4- | |
| | (trifluoromethyl)benzamide | |
| 140 | 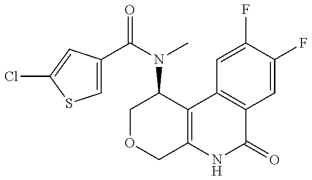 [SMILES: CN(C(=O)c1csc(Cl)c1)[C@@H]1COCc2[nH]c(=O)c3cc(F)c(F)cc3c21] | 0.10 |
| | (S)-5-Chloro-N-(8,9-difluoro-6-oxo-1,4,5,6-tetrahydro-2H- | |
| | pyrano[3,4-c]isoquinolin-1-yl)-N-methylthiophene-3-carboxamide | |
| 143 | 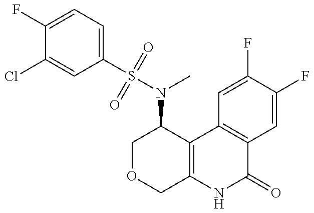 [SMILES: CN([C@@H]1COCc2[nH]c(=O)c3cc(F)c(F)cc3c21)S(=O)(=O)c1ccc(F)c(Cl)c1] | 18 |
| | C | |
| | (S)-3-Chloro-N-(8,9-difluoro-6-oxo-1,4,5,6-tetrahydro-2H- | |
| | pyrano[3,4-c]isoquinolin-1-yl)-4-fluoro-N-methylbenzenesulfonamide | |
| 144 | 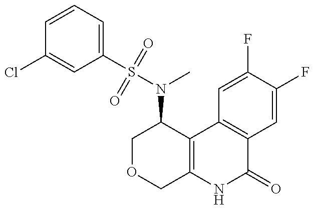 [SMILES: CN([C@@H]1COCc2[nH]c(=O)c3cc(F)c(F)cc3c21)S(=O)(=O)c1cccc(Cl)c1] | 12 |
| | (S)-3-Chloro-N-(8,9-difluoro-6-oxo-1,4,5,6-tetrahydro-2H- | |
| | pyrano[3,4-c]isoquinolin-1-yl)-N-methylbenzenesulfonamide | |
| 145 | 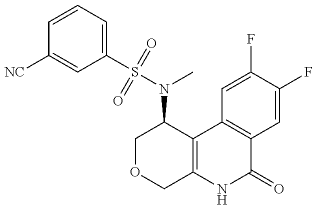 [SMILES: CN([C@@H]1COCc2[nH]c(=O)c3cc(F)c(F)cc3c21)S(=O)(=O)c1cccc(C#N)c1] | 5.5 |
| | (S)-3-Cyano-N-(8,9-difluoro-6-oxo-1,4,5,6-tetrahydro-2H-pyrano[3,4- | |
| | c]isoquinolin-1-yl)-N-methylbenzenesulfonamide | |
| 146 | 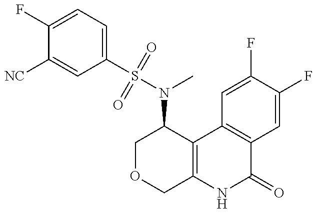 [SMILES: CN([C@@H]1COCc2[nH]c(=O)c3cc(F)c(F)cc3c21)S(=O)(=O)c1ccc(F)c(C#N)c1] | 23 |
| | (S)-3-Cyano-N-(8,9-difluoro-6-oxo-1,4,5,6-tetrahydro-2H-pyrano[3,4- | |
| | c]isoquinolin-1-yl)-4-fluoro-N-methylbenzenesulfonamide | |
| 147 | 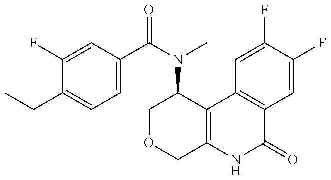 [SMILES: CCc1ccc(C(=O)N(C)[C@@H]2COCc3[nH]c(=O)c4cc(F)c(F)cc4c32)cc1F] | 3.3 |
| | (S)-N-(8,9-Difluoro-6-oxo-1,4,5,6-tetrahydro-2H-pyrano[3,4- | |
| | c]isoquinolin-1-y1)-4-ethyl-3-fluoro-N-methylbenzamide | |
| 148 | 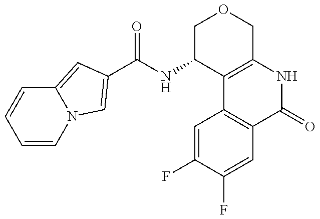 [SMILES: O=C(N[C@@H]1COCc2[nH]c(=O)c3cc(F)c(F)cc3c21)c1cc2ccccn2c1] | 0.76 |
| | (S)-N-(8,9-difluoro-6-oxo-1,4,5,6-tetrahydro-2H-pyrano[3,4-c]isoquinolin- | |
| | 1-y1)indolizine-2-carboxamide | |
| 149 | 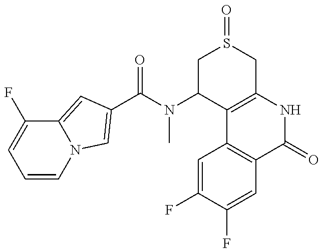 [SMILES: CN(C(=O)c1cc2c(F)cccn2c1)C1CS(=O)Cc2[nH]c(=O)c3cc(F)c(F)cc3c21] | 0.15 |
| | N-(8,9-difluoro-3-oxido-6-oxo-1,4,5,6-tetrahydro-2H-thiopyrano[3,4- | |
| | c]isoquinolin-1-yl)-8-fluoro-N-methylindolizine-2-carboxamide | |
| | racemic diastereomer I | |
| 150 | 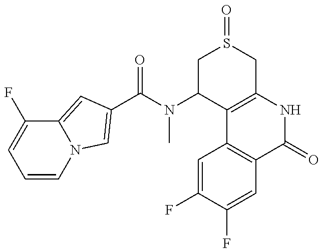 [SMILES: CN(C(=O)c1cc2c(F)cccn2c1)C1CS(=O)Cc2[nH]c(=O)c3cc(F)c(F)cc3c21] | 1.10 |
| | N-(8,9-difluoro-3-oxido-6-oxo-1,4,5,6-tetrahydro-2H-thiopyrano[3,4- | |
| | c]isoquinolin-1-yl)-8-fluoro-N-methylindolizine-2-carboxamide | |
| | racemic diastereomer II | |
| 151 | 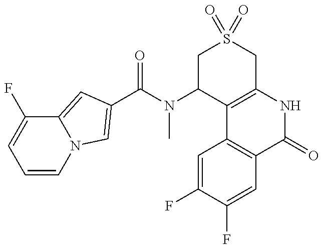 [SMILES: CN(C(=O)c1cc2c(F)cccn2c1)C1CS(=O)(=O)Cc2[nH]c(=O)c3cc(F)c(F)cc3c21] | 3.61 |
| | N-(8,9-difluoro-3,3-dioxido-6-oxo-1,4,5,6-tetrahydro-2H-thiopyrano[3,4- | |
| | c]isoquinolin-1-y1)-8-fluoro-N-methylindolizine-2-carboxamide | |
| | enantiomer I | |
| 152 | 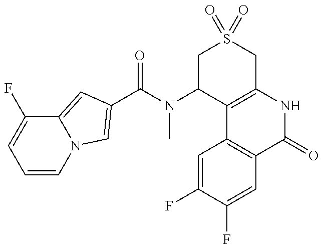 [SMILES: CN(C(=O)c1cc2c(F)cccn2c1)C1CS(=O)(=O)Cc2[nH]c(=O)c3cc(F)c(F)cc3c21] | 0.73 |
| | N-(8,9-difluoro-3,3-dioxido-6-oxo-1,4,5,6-tetrahydro-2H-thiopyrano[3,4- | |
| | c]isoquinolin-1-yl)-8-fluoro-N-methylindolizine-2-carboxamide | |
| | enantiomer II | |
| 153 | 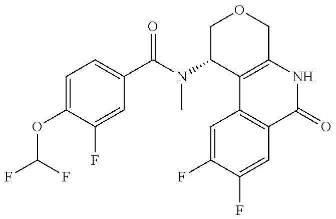 [SMILES: CN(C(=O)c1ccc(OC(F)F)c(F)c1)[C@@H]1COCc2[nH]c(=O)c3cc(F)c(F)cc3c21] | 0.15 |
| | (S)-N-(8,9-difluoro-6-oxo-1,4,5,6-tetrahydro-2H-pyrano[3,4-c]isoquinolin- | |
| | 1-y1)-4-(difluoromethoxy)-3-fluoro-N-methylbenzamide | |
| 154 | 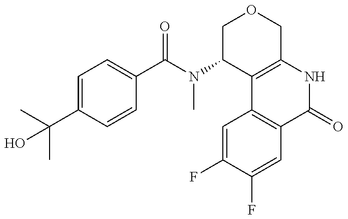 [SMILES: CN(C(=O)c1ccc(C(C)(C)O)cc1)[C@@H]1COCc2[nH]c(=O)c3cc(F)c(F)cc3c21] | 1.05 |
| | (S)-N-(8,9-difluoro-6-oxo-1,4,5,6-tetrahydro-2H-pyrano[3,4-c]isoquinolin- | |
| | 1-y1)-4-(2-hydroxypropan-2-yl)-N-methylbenzamide | |
| 155 | 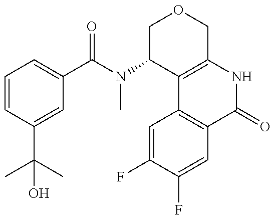 [SMILES: CN(C(=O)c1cccc(C(C)(C)O)c1)[C@@H]1COCc2[nH]c(=O)c3cc(F)c(F)cc3c21] | 4.49 |
| | (S)-N-(8,9-difluoro-6-oxo-1,4,5,6-tetrahydro-2H-pyrano[3,4-c]isoquinolin- | |
| | 1-yl)-3-(2-hydroxypropan-2-y1)-N-methylbenzamide | |
| 156 | 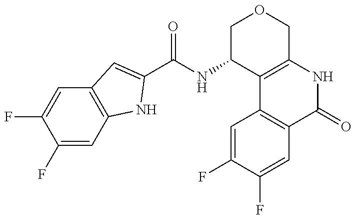 [SMILES: O=C(N[C@@H]1COCc2[nH]c(=O)c3cc(F)c(F)cc3c21)c1cc2cc(F)c(F)cc2[nH]1] | 0.49 |
| | (S)-N-(8,9-difluoro-6-oxo-1,4,5,6-tetrahydro-2H-pyrano[3,4-c]isoquinolin- | |
| | 1-y1)-5,6-difluoro-1H-indole-2-carboxamide | |
| 157 | 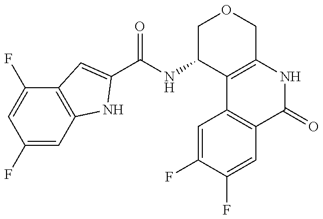 [SMILES: O=C(N[C@@H]1COCc2[nH]c(=O)c3cc(F)c(F)cc3c21)c1cc2c(F)cc(F)cc2[nH]1] | 0.25 |
| | (S)-N-(8,9-difluoro-6-oxo-1,4,5,6-tetrahydro-2H-pyrano[3,4-c]isoquinolin- | |
| | 1-yl)-4,6-difluoro-1H-indole-2-carboxamide | |
| 158 | 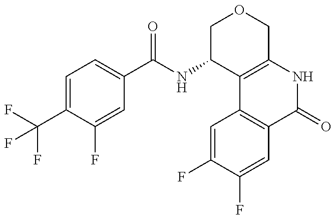 [SMILES: O=C(N[C@@H]1COCc2[nH]c(=O)c3cc(F)c(F)cc3c21)c1ccc(C(F)(F)F)c(F)c1] | 0.58 |
| | (S)-N-(8,9-difluoro-6-oxo-1,4,5,6-tetrahydro-2H-pyrano[3,4-c]isoquinolin- | |
| | 1-yl)-3-fluoro-4-(trifluoromethyl)benzamide | |
| 159 | 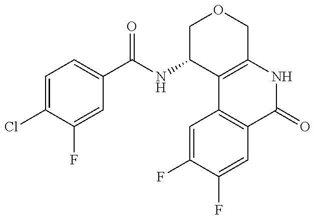 [SMILES: O=C(N[C@@H]1COCc2[nH]c(=O)c3cc(F)c(F)cc3c21)c1ccc(Cl)c(F)c1] | 0.66 |
| | (S)-4-chloro-N-(8,9-difluoro-6-oxo-1,4,5,6-tetrahydro-2H-pyrano[3,4- | |
| | c]isoquinolin-1-yl)-3-fluorobenzamide | |
| 160 | 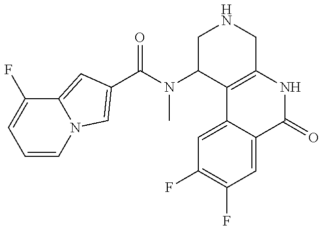 [SMILES: CN(C(=O)c1cc2c(F)cccn2c1)C1CNCc2[nH]c(=O)c3cc(F)c(F)cc3c21] | 16.20 |
| | N-(8,9-difluoro-6-oxo-1,2,3,4,5,6-hexahydrobenzo[c][1,7]naphthyridin-1- | |
| | y1)-8-fluoro-N-methylindolizine-2-carboxamide | |
| | enantiomer I | |
| 161 | 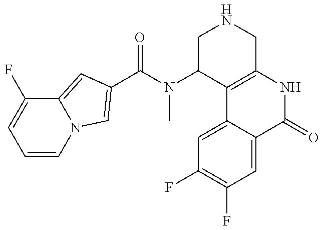 [SMILES: CN(C(=O)c1cc2c(F)cccn2c1)C1CNCc2[nH]c(=O)c3cc(F)c(F)cc3c21] | 0.02 |
| | N-(8,9-difluoro-6-oxo-1,2,3,4,5,6-hexahydrobenzo[c][1,7]naphthyridin-1- | |
| | yl)-8-fluoro-N-methylindolizine-2-carboxamide | |
| | enantiomer II | |
| 162 | 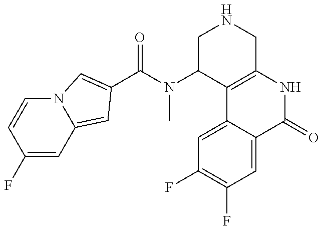 [SMILES: CN(C(=O)c1cc2cc(F)ccn2c1)C1CNCc2[nH]c(=O)c3cc(F)c(F)cc3c21] | 13.82 |
| | N-(8,9-difluoro-6-oxo-1,2,3,4,5,6-hexahydrobenzo[c][1,7]naphthyridin-1- | |
| | yl)-7-fluoro-N-methylindolizine-2-carboxamide | |
| | enantiomer I | |
| 163 | 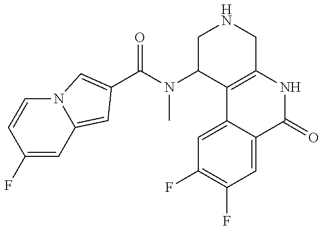 [SMILES: CN(C(=O)c1cc2cc(F)ccn2c1)C1CNCc2[nH]c(=O)c3cc(F)c(F)cc3c21] | 0.03 |
| | N-(8,9-difluoro-6-oxo-1,2,3,4,5,6-hexahydrobenzo[c][1,7]naphthyridin-1- | |
| | yl)-7-fluoro-N-methylindolizine-2-carboxamide | |
| | enantiomer II | |
| 164 | 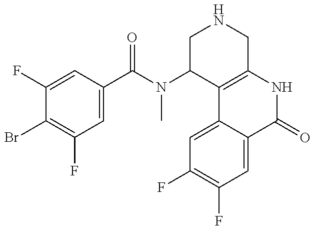 [SMILES: CN(C(=O)c1cc(F)c(Br)c(F)c1)C1CNCc2[nH]c(=O)c3cc(F)c(F)cc3c21] | 2.88 |
| | 4-bromo-N-(8,9-difluoro-6-oxo-1,2,3,4,5,6- | |
| | hexahydrobenzo[c][1,7]naphthyridin-1-yl)-3,5-difluoro-N-methylbenzamide | |
| | enantiomer I | |
| 165 | 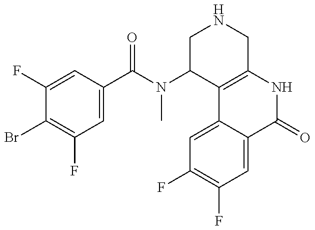 [SMILES: CN(C(=O)c1cc(F)c(Br)c(F)c1)C1CNCc2[nH]c(=O)c3cc(F)c(F)cc3c21] | 0.02 |
| | 4-bromo-N-(8,9-difluoro-6-oxo-1,2,3,4,5,6- | |
| | hexahydrobenzo[c][1,7]naphthyridin-1-yl)-3,5-difluoro-N-methylbenzamide | |
| | enantiomer II | |
| 166 | 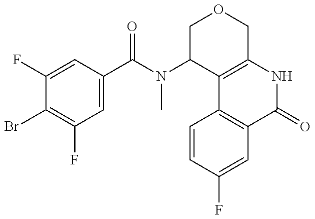 [SMILES: CN(C(=O)c1cc(F)c(Br)c(F)c1)C1COCc2[nH]c(=O)c3cc(F)ccc3c21] | 1.91 |
| | 4-bromo-3,5-difluoro-N-(8-fluoro-6-oxo-1,4,5,6-tetrahydro-2H-pyrano[3,4- | |
| | c]isoquinolin-1-y1)-N-methylbenzamide | |
| | enantiomer I | |
| 167 | 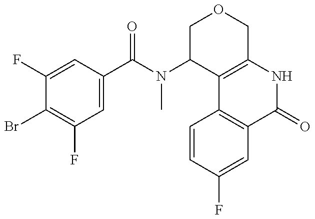 [SMILES: CN(C(=O)c1cc(F)c(Br)c(F)c1)C1COCc2[nH]c(=O)c3cc(F)ccc3c21] | 0.01 |
| | 4-bromo-3,5-difluoro-N-(8-fluoro-6-oxo-1,4,5,6-tetrahydro-2H-pyrano[3,4- | |
| | c]isoquinolin-1-y1)-N-methylbenzamide | |
| | enantiomer II | |
| 168 | 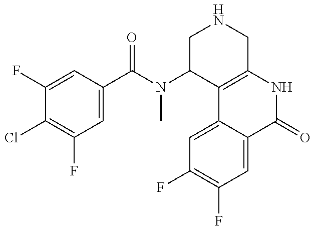 [SMILES: CN(C(=O)c1cc(F)c(Cl)c(F)c1)C1CNCc2[nH]c(=O)c3cc(F)c(F)cc3c21] | 11.3 |
| | 4-chloro-N-(8,9-difluoro-6-oxo-1,2,3,4,5,6- | |
| | hexahydrobenzo[c][1,7]naphthyridin-1-yl)-3,5-difluoro-N-methylbenzamide | |
| | enantiomer I | |
| 169 | 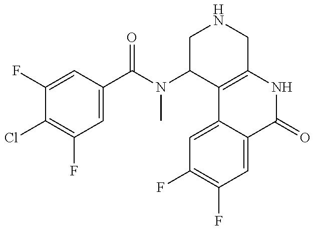 [SMILES: CN(C(=O)c1cc(F)c(Cl)c(F)c1)C1CNCc2[nH]c(=O)c3cc(F)c(F)cc3c21] | 0.04 |
| | 4-chloro-N-(8,9-difluoro-6-oxo-1,2,3,4,5,6- | |
| | hexahydrobenzo[c][1,7]naphthyridin-1-y1)-3,5-difluoro-N-methylbenzamide | |
| | enantiomer II | |
| 170 | 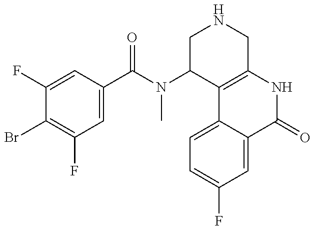 [SMILES: CN(C(=O)c1cc(F)c(Br)c(F)c1)C1CNCc2[nH]c(=O)c3cc(F)ccc3c21] | 22 |
| | 4-bromo-3,5-difluoro-N-(8-fluoro-6-oxo-1,2,3,4,5,6- | |
| | hexahydrobenzo[c][1,7]naphthyridin-1-yl)-N-methylbenzamide | |
| | enantiomer I | |
| 171 | 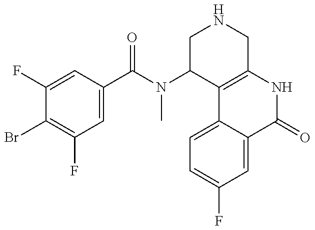 [SMILES: CN(C(=O)c1cc(F)c(Br)c(F)c1)C1CNCc2[nH]c(=O)c3cc(F)ccc3c21] | 0.51 |
| | 4-bromo-3,5-difluoro-N-(8-fluoro-6-oxo-1,2,3,4,5,6- | |
| | hexahydrobenzo[c][1,7]naphthyridin-1-yl)-N-methylbenzamide | |
| | enantiomer II | |
| 172 | 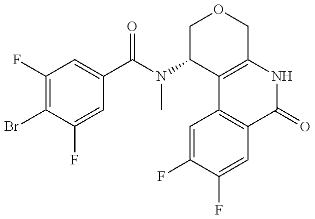 [SMILES: CN(C(=O)c1cc(F)c(Br)c(F)c1)[C@@H]1COCc2[nH]c(=O)c3cc(F)c(F)cc3c21] | 0.01 |
| | (S)-4-bromo-N-(8,9-difluoro-6-oxo-1,4,5,6-tetrahydro-2H-pyrano[3,4- | |
| | c]isoquinolin-1-yl)-3,5-difluoro-N-methylbenzamide | |
| 173 | 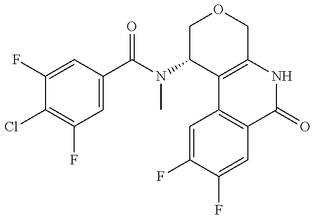 [SMILES: CN(C(=O)c1cc(F)c(Cl)c(F)c1)[C@@H]1COCc2[nH]c(=O)c3cc(F)c(F)cc3c21] | 0.01 |
| | (S)-4-chloro-N-(8,9-difluoro-6-oxo-1,4,5,6-tetrahydro-2H-pyrano[3,4- | |
| | c]isoquinolin-1-yl)-3,5-difluoro-N-methylbenzamide | |
| 174 | 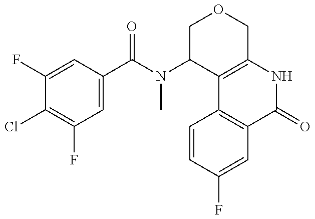 [SMILES: CN(C(=O)c1cc(F)c(Cl)c(F)c1)C1COCc2[nH]c(=O)c3cc(F)ccc3c21] | 5.1 |
| | 4-chloro-3,5-difluoro-N-(8-fluoro-6-oxo-1,4,5,6-tetrahydro-2H-pyrano[3,4- | |
| | c]isoquinolin-1-y1)-N-methylbenzamide | |
| | enantiomer I | |
| 175 | 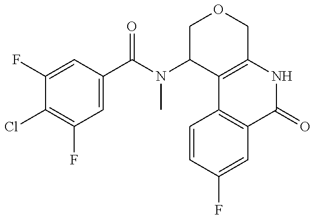 [SMILES: CN(C(=O)c1cc(F)c(Cl)c(F)c1)C1COCc2[nH]c(=O)c3cc(F)ccc3c21] | 0.03 |
| | 4-chloro-3,5-difluoro-N-(8-fluoro-6-oxo-1,4,5,6-tetrahydro-2H-pyrano[3,4- | |
| | c]isoquinolin-1-yl)-N-methylbenzamide | |
| | enantiomer II | |
| 176 | 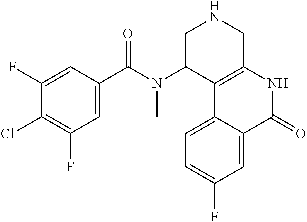 [SMILES: CN(C(=O)c1cc(F)c(Cl)c(F)c1)C1CNCc2[nH]c(=O)c3cc(F)ccc3c21] | 19.6 |
| | 4-chloro-3,5-difluoro-N-(8-fluoro-6-oxo-1,2,3,4,5,6- | |
| | hexahydrobenzo[c][1,7]naphthyridin-1-yl)-N-methylbenzamide | |
| | enantiomer I | |
| 177 | 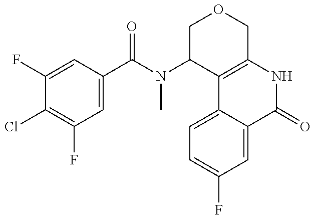 [SMILES: CN(C(=O)c1cc(F)c(Cl)c(F)c1)C1COCc2[nH]c(=O)c3cc(F)ccc3c21] | 0.15 |
| | 4-chloro-3,5-difluoro-N-(8-fluoro-6-oxo-1,2,3,4,5,6- | |
| | hexahydrobenzo[c][1,7]naphthyridin-1-yl)-N-methylbenzamide | |
| | enantiomer II | |
| 178 | 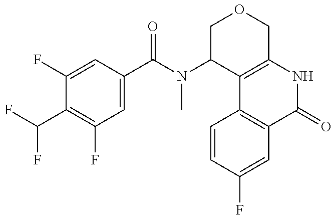 [SMILES: CN(C(=O)c1cc(F)c(C(F)F)c(F)c1)C1COCc2[nH]c(=O)c3cc(F)ccc3c21] | 7.4 |
| | 4-(difluoromethyl)-3,5-difluoro-N-(8-fluoro-6-oxo-1,4,5,6-tetrahydro-2H- | |
| | pyrano[3,4-c]isoquinolin-1-yl)-N-methylbenzamide | |
| | enantiomer I | |
| 179 | 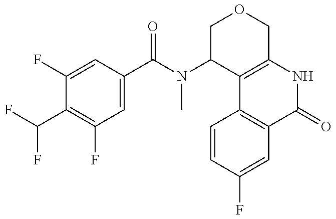 [SMILES: CN(C(=O)c1cc(F)c(C(F)F)c(F)c1)C1COCc2[nH]c(=O)c3cc(F)ccc3c21] | 0.05 |
| | 4-(difluoromethyl)-3,5-difluoro-N-(8-fluoro-6-oxo-1,4,5,6-tetrahydro-2H- | |
| | pyrano[3,4-c]isoquinolin-1-y1)-N-methylbenzamide | |
| | enantiomer II | |
| 180 | 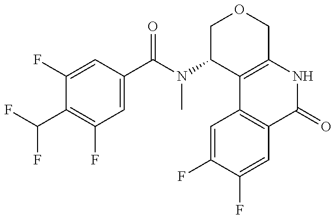 [SMILES: CN(C(=O)c1cc(F)c(C(F)F)c(F)c1)[C@@H]1COCc2[nH]c(=O)c3cc(F)c(F)cc3c21] | 0.16 |
| | (S)-N-(8,9-difluoro-6-oxo-1,4,5,6-tetrahydro-2H-pyrano[3,4-c]isoquinolin- | |
| | 1-yl)-4-(difluoromethyl)-3,5-difluoro-N-methylbenzamide | |
| 181 | 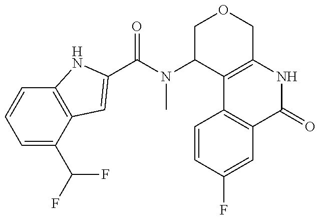 [SMILES: CN(C(=O)c1cc2c(C(F)F)cccc2[nH]1)C1COCc2[nH]c(=O)c3cc(F)ccc3c21] | 5.5 |
| | 4-(difluoromethyl)-N-(8-fluoro-6-oxo-1,4,5,6-tetrahydro-2H-pyrano[3,4- | |
| | c]isoquinolin-1-yl)-N-methyl-1H-indole-2-carboxamide | |
| | enantiomer I | |
| 182 | 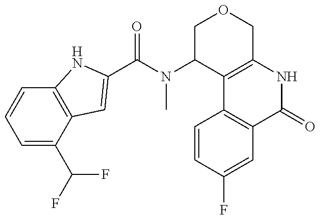 [SMILES: CN(C(=O)c1cc2c(C(F)F)cccc2[nH]1)C1COCc2[nH]c(=O)c3cc(F)ccc3c21] | 0.75 |
| | 4-(difluoromethyl)-N-(8-fluoro-6-oxo-1,4,5,6-tetrahydro-2H-pyrano[3,4- | |
| | c]isoquinolin-1-yl)-N-methyl-1H-indole-2-carboxamide | |
| | enantiomer II | |
| 183 | 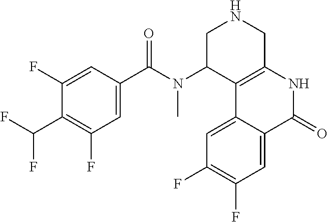 [SMILES: CN(C(=O)c1cc(F)c(C(F)F)c(F)c1)C1CNCc2[nH]c(=O)c3cc(F)c(F)cc3c21] | 25.00 |
| | N-(8,9-difluoro-6-oxo-1,2,3,4,5,6-hexahydrobenzo[c][1,7]naphthyridin-1- | |
| | yl)-4-(difluoromethyl)-3,5-difluoro-N-methylbenzamide | |
| | enantiomer I | |
| 184 | 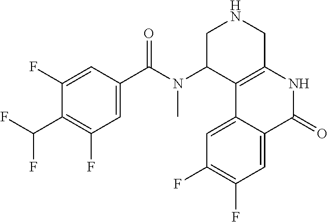 [SMILES: CN(C(=O)c1cc(F)c(C(F)F)c(F)c1)C1CNCc2[nH]c(=O)c3cc(F)c(F)cc3c21] | 0.07 |
| | N-(8,9-difluoro-6-oxo-1,2,3,4,5,6-hexahydrobenzo[c][1,7]naphthyridin-1- | |
| | yl)-4-(difluoromethyl)-3,5-difluoro-N-methylbenzamide | |
| | enantiomer II | |
| 185 | 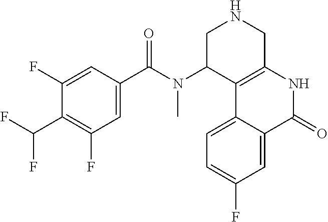 [SMILES: CN(C(=O)c1cc(F)c(C(F)F)c(F)c1)C1CNCc2[nH]c(=O)c3cc(F)ccc3c21] | 24 |
| | 4-(difluoromethyl)-3,5-difluoro-N-(8-fluoro-6-oxo-1,2,3,4,5,6- | |
| | hexahydrobenzo[c][1,7]naphthyridin-1-y1)-N-methylbenzamide | |
| | enantiomer I | |
| 186 | 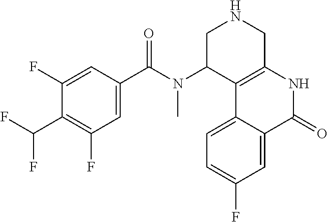 [SMILES: CN(C(=O)c1cc(F)c(C(F)F)c(F)c1)C1CNCc2[nH]c(=O)c3cc(F)ccc3c21] | 0.20 |
| | 4-(difluoromethyl)-3,5-difluoro-N-(8-fluoro-6-oxo-1,2,3,4,5,6- | |
| | hexahydrobenzo[c][1,7]naphthyridin-1-yl)-N-methylbenzamide | |
| | enantiomer II | |
| 187 | 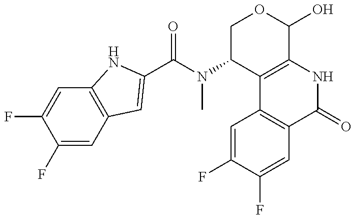 [SMILES: CN(C(=O)c1cc2cc(F)c(F)cc2[nH]1)[C@@H]1COC(O)c2[nH]c(=O)c3cc(F)c(F)cc3c21] | 0.01 |
| | N-((1S)-8,9-difluoro-4-hydroxy-6-oxo-1,4,5,6-tetrahydro-2H-pyrano[3,4- | |
| | c]isoquinolin-1-yl)-5,6-difluoro-N-methyl-1H-indole-2-carboxamide | |
| | ~6:1 ratio of epimers at anomeric carbon | |
| 188 | 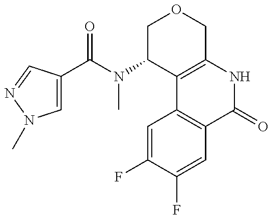 [SMILES: CN(C(=O)c1cnn(C)c1)[C@@H]1COCc2[nH]c(=O)c3cc(F)c(F)cc3c21] | 18.3 |
| | (S)-N-(8,9-difluoro-6-oxo-1,4,5,6-tetrahydro-2H-pyrano[3,4-c]isoquinolin- | |
| | 1-yl)-N,1-dimethyl-1H-pyrazole-4-carboxamide | |
| 189 | 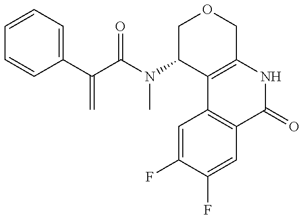 [SMILES: C=C(C(=O)N(C)[C@@H]1COCc2[nH]c(=O)c3cc(F)c(F)cc3c21)c1ccccc1] | 4.4 |
| | (S)-N-(8,9-difluoro-6-oxo-1,4,5,6-tetrahydro-2H-pyrano[3,4-c]isoquinolin- | |
| | 1-yl)-N-methyl-2-phenylacrylamide | |
| 190 | 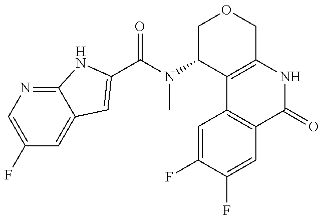 [SMILES: CN(C(=O)c1cc2cc(F)cnc2[nH]1)[C@@H]1COCc2[nH]c(=O)c3cc(F)c(F)cc3c21] | 0.33 |
| | (S)-N-(8,9-difluoro-6-oxo-1,4,5,6-tetrahydro-2H-pyrano[3,4-c]isoquinolin- | |
| | 1-yl)-5-fluoro-N-methyl-1H-pyrrolo[2,3-b]pyridine-2-carboxamide | |
| 191 | 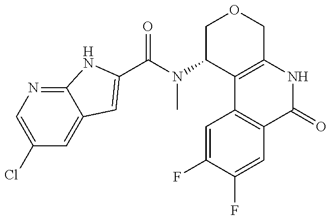 [SMILES: CN(C(=O)c1cc2cc(Cl)cnc2[nH]1)[C@@H]1COCc2[nH]c(=O)c3cc(F)c(F)cc3c21] | 0.12 |
| | (S)-5-chloro-N-(8,9-difluoro-6-oxo-1,4,5,6-tetrahydro-2H-pyrano[3,4- | |
| | c]isoquinolin-1-yl)-N-methyl-1H-pyrrolo[2,3-b]pyridine-2-carboxamide | |
| 192 | 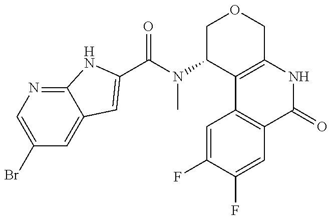 [SMILES: CN(C(=O)c1cc2cc(Br)cnc2[nH]1)[C@@H]1COCc2[nH]c(=O)c3cc(F)c(F)cc3c21] | 0.26 |
| | (S)-5-bromo-N-(8,9-difluoro-6-oxo-1,4,5,6-tetrahydro-2H-pyrano[3,4- | |
| | c]isoquinolin-1-yl)-N-methyl-1H-pyrrolo[2,3-b]pyridine-2-carboxamide | |
| 193 | 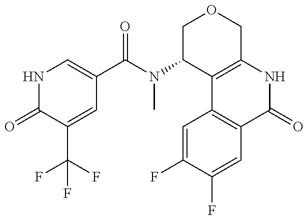 [SMILES: CN(C(=O)c1c[nH]c(=O)c(C(F)(F)F)c1)[C@@H]1COCc2[nH]c(=O)c3cc(F)c(F)cc3c21] | 3.2 |
| | (S)-N-(8,9-difluoro-6-oxo-1,4,5,6-tetrahydro-2H-pyrano[3,4-c]isoquinolin- | |
| | 1-yl)-N-methyl-6-oxo-5-(trifluoromethyl)-1,6-dihydropyridine-3- | |
| | carboxamide | |
| 194 | 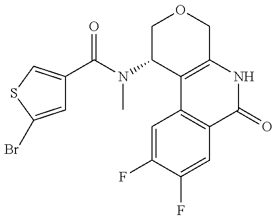 [SMILES: CN(C(=O)c1csc(Br)c1)[C@@H]1COCc2[nH]c(=O)c3cc(F)c(F)cc3c21] | 0.07 |
| | (S)-5-bromo-N-(8,9-difluoro-6-oxo-1,4,5,6-tetrahydro-2H-pyrano[3,4- | |
| | c]isoquinolin-1-yl)-N-methylthiophene-3-carboxamide | |
| 195 | 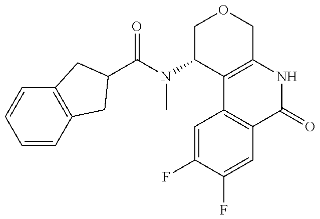 [SMILES: CN(C(=O)C1Cc2ccccc2C1)[C@@H]1COCc2[nH]c(=O)c3cc(F)c(F)cc3c21] | 0.15 |
| | (S)-N-(8,9-difluoro-6-oxo-1,4,5,6-tetrahydro-2H-pyrano[3,4-c]isoquinolin- | |
| | 1-y1)-N-methyl-2,3-dihydro-1H-indene-2-carboxamide | |
| 196 | 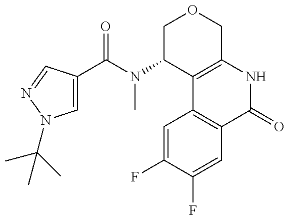 [SMILES: CN(C(=O)c1cnn(C(C)(C)C)c1)[C@@H]1COCc2[nH]c(=O)c3cc(F)c(F)cc3c21] | 3.1 |
| | (S)-1-(tert-butyl)-N-(8,9-difluoro-6-oxo-1,4,5,6-tetrahydro-2H-pyrano[3,4- | |
| | c]isoquinolin-1-yl)-N-methyl-1H-pyrazole-4-carboxamide | |
| 197 | 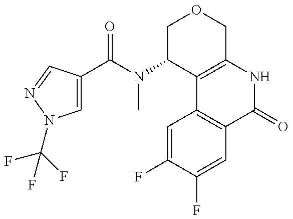 [SMILES: CN(C(=O)c1cnn(C(F)(F)F)c1)[C@@H]1COCc2[nH]c(=O)c3cc(F)c(F)cc3c21] | 0.44 |
| | (S)-N-(8,9-difluoro-6-oxo-1,4,5,6-tetrahydro-2H-pyrano[3,4-c]isoquinolin- | |
| | 1-yl)-N-methyl-1-(trifluoromethyl)-1H-pyrazole-4-carboxamide | |
| 198 | 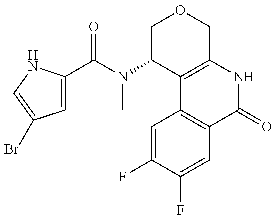 [SMILES: CN(C(=O)c1cc(Br)c[nH]1)[C@@H]1COCc2[nH]c(=O)c3cc(F)c(F)cc3c21] | 0.22 |
| | (S)-4-bromo-N-(8,9-difluoro-6-oxo-1,4,5,6-tetrahydro-2H-pyrano[3,4- | |
| | c]isoquinolin-1-yl)-N-methyl-1H-pyrrole-2-carboxamide | |
| 199 | 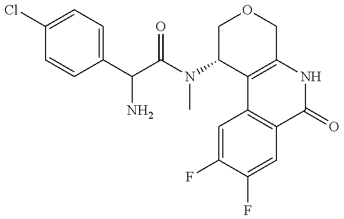 [SMILES: CN(C(=O)C(N)c1ccc(Cl)cc1)[C@@H]1COCc2[nH]c(=O)c3cc(F)c(F)cc3c21] | 14.7 |
| | 2-amino-2-(4-chlorophenyl)-N-((S)-8,9-difluoro-6-oxo-1,4,5,6-tetrahydro- | |
| | 2H-pyrano[3,4-c]isoquinolin-1-yl)-N-methylacetamide | |
| | diastereomer I | |
| 201 | 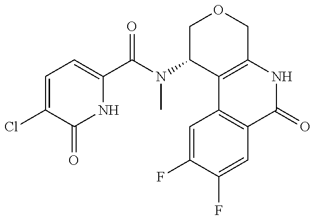 [SMILES: CN(C(=O)c1ccc(Cl)c(=O)[nH]1)[C@@H]1COCc2[nH]c(=O)c3cc(F)c(F)cc3c21] | 10 |
| | (S)-5-chloro-N-(8,9-difluoro-6-oxo-1,4,5,6-tetrahydro-2H-pyrano[3,4- | |
| | c]isoquinolin-1-yl)-N-methyl-6-oxo-1,6-dihydropyridine-2-carboxamide | |
| 202 | 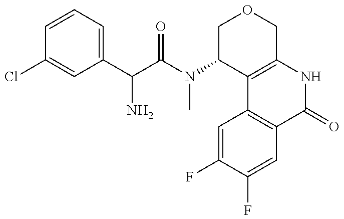 [SMILES: CN(C(=O)C(N)c1cccc(Cl)c1)[C@@H]1COCc2[nH]c(=O)c3cc(F)c(F)cc3c21] | 16 |
| | 2-amino-2-(3-chlorophenyl)-N-((S)-8,9-difluoro-6-oxo-1,4,5,6-tetrahydro- | |
| | 2H-pyrano[3,4-c]isoquinolin-1-yl)-N-methylacetamide | |
| | diastereomer I | |
| 204 | 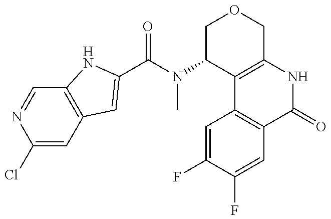 [SMILES: CN(C(=O)c1cc2cc(Cl)ncc2[nH]1)[C@@H]1COCc2[nH]c(=O)c3cc(F)c(F)cc3c21] | 0.04 |
| | (S)-5-chloro-N-(8,9-difluoro-6-oxo-1,4,5,6-tetrahydro-2H-pyrano[3,4- | |
| | c]isoquinolin-1-yl)-N-methyl-1H-pyrrolo[2,3-c]pyridine-2-carboxamide | |
| 205 | 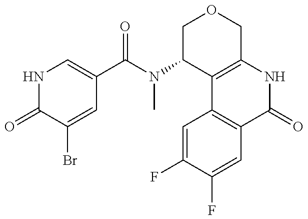 [SMILES: CN(C(=O)c1c[nH]c(=O)c(Br)c1)[C@@H]1COCc2[nH]c(=O)c3cc(F)c(F)cc3c21] | 15.8 |
| | (S)-5-bromo-N-(8,9-difluoro-6-oxo-1,4,5,6-tetrahydro-2H-pyrano[3,4- | |
| | c]isoquinolin-1-yl)-N-methyl-6-oxo-1,6-dihydropyridine-3-carboxamide | |
| 206 | 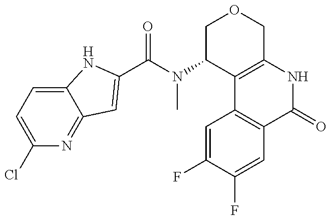 [SMILES: CN(C(=O)c1cc2nc(Cl)ccc2[nH]1)[C@@H]1COCc2[nH]c(=O)c3cc(F)c(F)cc3c21] | 0.63 |
| | (S)-5-chloro-N-(8,9-difluoro-6-oxo-1,4,5,6-tetrahydro-2H-pyrano[3,4- | |
| | c]isoquinolin-1-yl)-N-methyl-1H-pyrrolo[3,2-b]pyridine-2-carboxamide | |
| 207 | 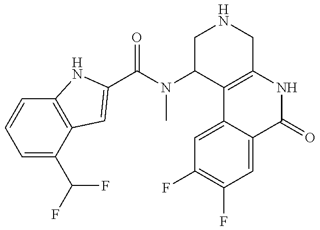 [SMILES: CN(C(=O)c1cc2c(C(F)F)cccc2[nH]1)C1CNCc2[nH]c(=O)c3cc(F)c(F)cc3c21] | 5.6 |
| | N-(8,9-difluoro-6-oxo-1,2,3,4,5,6-hexahydrobenzo[c][1,7]naphthyridin-1- | |
| | yl)-4-(difluoromethyl)-N-methyl-1H-indole-2-carboxamide | |
| | enantiomer I | |
| 208 | 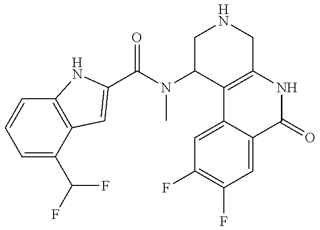 [SMILES: CN(C(=O)c1cc2c(C(F)F)cccc2[nH]1)C1CNCc2[nH]c(=O)c3cc(F)c(F)cc3c21] | 0.03 |
| | N-(8,9-difluoro-6-oxo-1,2,3,4,5,6-hexahydrobenzo[c][1,7]naphthyridin-1- | |
| | y1)-4-(difluoromethyl)-N-methyl-1H-indole-2-carboxamide | |
| | enantiomer II | |
| 209 | 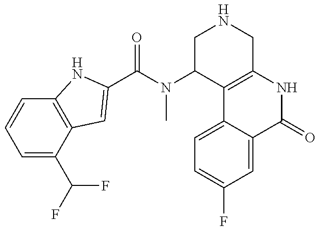 [SMILES: CN(C(=O)c1cc2c(C(F)F)cccc2[nH]1)C1CNCc2[nH]c(=O)c3cc(F)ccc3c21] | 4.4 |
| | 4-(difluoromethyl)-N-(8-fluoro-6-oxo-1,2,3,4,5,6- | |
| | hexahydrobenzo[c][1,7]naphthyridin-1-yl)-N-methyl-1H-indole-2- | |
| | carboxamide | |
| | enantiomer I | |
| 210 | 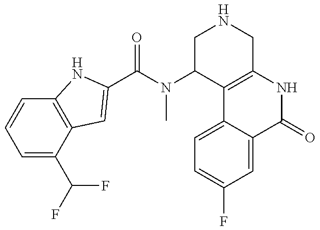 [SMILES: CN(C(=O)c1cc2c(C(F)F)cccc2[nH]1)C1CNCc2[nH]c(=O)c3cc(F)ccc3c21] | 0.04 |
| | 4-(difluoromethyl)-N-(8-fluoro-6-oxo-1,2,3,4,5,6- | |
| | hexahydrobenzo[c][1,7]naphthyridin-1-yl)-N-methyl-1H-indole-2- | |
| | carboxamide | |
| | enantiomer II | |
| 211 | 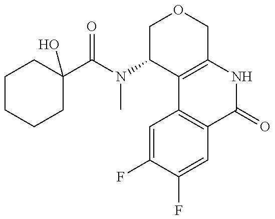 [SMILES: CN(C(=O)C1(O)CCCCC1)[C@@H]1COCc2[nH]c(=O)c3cc(F)c(F)cc3c21] | 7.66 |
| | (S)-N-(8,9-difluoro-6-oxo-1,4,5,6-tetrahydro-2H-pyrano[3,4-c]isoquinolin- | |
| | 1-y1)-1-hydroxy-N-methylcyclohexane-1-carboxamide | |
| 212 | 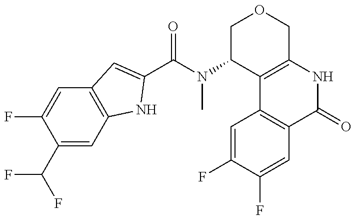 [SMILES: CN(C(=O)c1cc2cc(F)c(C(F)F)cc2[nH]1)[C@@H]1COCc2[nH]c(=O)c3cc(F)c(F)cc3c21] | 0.01 |
| | (S)-N-(8,9-difluoro-6-oxo-1,4,5,6-tetrahydro-2H-pyrano[3,4-c]isoquinolin- | |
| | 1-yl)-6-(difluoromethyl)-5-fluoro-N-methyl-1H-indole-2-carboxamide | |
| 213 | 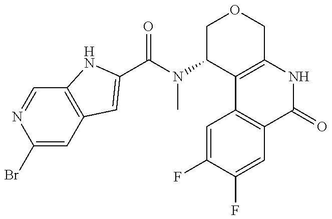 [SMILES: CN(C(=O)c1cc2cc(Br)ncc2[nH]1)[C@@H]1COCc2[nH]c(=O)c3cc(F)c(F)cc3c21] | 0.08 |
| | (S)-5-bromo-N-(8,9-difluoro-6-oxo-1,4,5,6-tetrahydro-2H-pyrano[3,4- | |
| | c]isoquinolin-1-yl)-N-methyl-1H-pyrrolo[2,3-c]pyridine-2-carboxamide | |
| 214 | 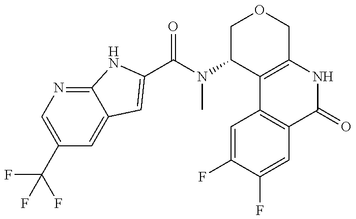 [SMILES: CN(C(=O)c1cc2cc(C(F)(F)F)cnc2[nH]1)[C@@H]1COCc2[nH]c(=O)c3cc(F)c(F)cc3c21] | 1.00 |
| | (S)-N-(8,9-difluoro-6-oxo-1,4,5,6-tetrahydro-2H-pyrano[3,4-c]isoquinolin- | |
| | 1-yl)-N-methyl-5-(trifluoromethyl)-1H-pyrrolo[2,3-b]pyridine-2- | |
| | carboxamide | |
| 215 | 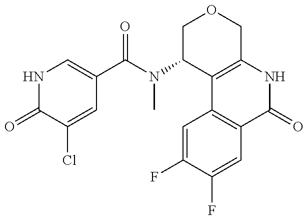 [SMILES: CN(C(=O)c1c[nH]c(=O)c(Cl)c1)[C@@H]1COCc2[nH]c(=O)c3cc(F)c(F)cc3c21] | 20.1 |
| | (S)-5-chloro-N-(8,9-difluoro-6-oxo-1,4,5,6-tetrahydro-2H-pyrano[3,4- | |
| | c]isoquinolin-1-yl)-N-methyl-6-oxo-1,6-dihydropyridine-3-carboxamide | |
| 216 | 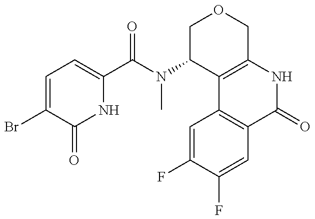 [SMILES: CN(C(=O)c1ccc(Br)c(=O)[nH]1)[C@@H]1COCc2[nH]c(=O)c3cc(F)c(F)cc3c21] NH | 7.3 |
| | (S)-5-bromo-N-(8,9-difluoro-6-oxo-1,4,5,6-tetrahydro-2H-pyrano[3,4- | |
| | c]isoquinolin-1-yl)-N-methyl-6-oxo-1,6-dihydropyridine-2-carboxamide | |
| 217 | 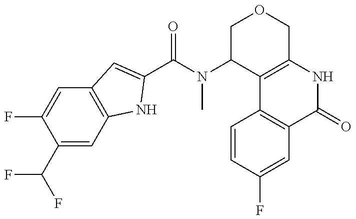 [SMILES: CN(C(=O)c1cc2cc(F)c(C(F)F)cc2[nH]1)C1COCc2[nH]c(=O)c3cc(F)ccc3c21] | 0.88 |
| | 6-(difluoromethyl)-5-fluoro-N-(8-fluoro-6-oxo-1,4,5,6-tetrahydro-2H- | |
| | pyrano[3,4-c]isoquinolin-1-yl)-N-methyl-1H-indole-2-carboxamide | |
| | enantiomer I | |
| 218 | 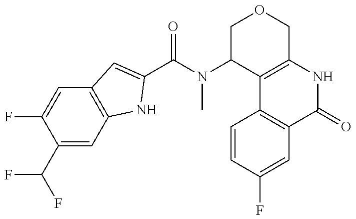 [SMILES: CN(C(=O)c1cc2cc(F)c(C(F)F)cc2[nH]1)C1COCc2[nH]c(=O)c3cc(F)ccc3c21] | 0.03 |
| | 6-(difluoromethyl)-5-fluoro-N-(8-fluoro-6-oxo-1,4,5,6-tetrahydro-2H- | |
| | pyrano[3,4-c]isoquinolin-1-yl)-N-methyl-1H-indole-2-carboxamide | |
| | enantiomer II | |
| 219 | 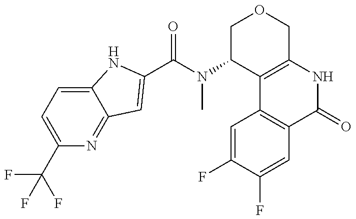 [SMILES: CN(C(=O)c1cc2nc(C(F)(F)F)ccc2[nH]1)[C@@H]1COCc2[nH]c(=O)c3cc(F)c(F)cc3c21] | 1.44 |
| | (S)-N-(8,9-difluoro-6-oxo-1,4,5,6-tetrahydro-2H-pyrano[3,4-c]isoquinolin- | |
| | 1-yl)-N-methyl-5-(trifluoromethyl)-1H-pyrrolo[3,2-b]pyridine-2- | |
| | carboxamide | |
| 220 | 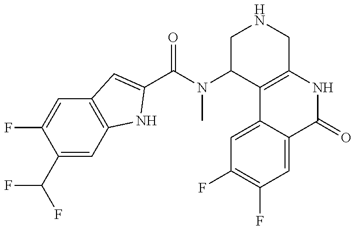 [SMILES: CN(C(=O)c1cc2cc(F)c(C(F)F)cc2[nH]1)C1CNCc2[nH]c(=O)c3cc(F)c(F)cc3c21] | 2.45 |
| | N-(8,9-difluoro-6-oxo-1,2,3,4,5,6-hexahydrobenzo[c][1,7]naphthyridin- | |
| | 1- yl)-6-(difluoromethyl)-5-fluoro-N-methyl-1H-indole-2-carboxamide | |
| | enantiomer I | |
| 221 | 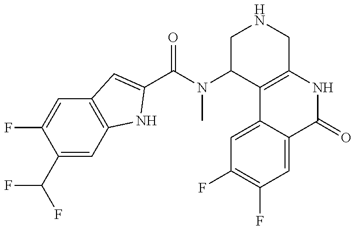 [SMILES: CN(C(=O)c1cc2cc(F)c(C(F)F)cc2[nH]1)C1CNCc2[nH]c(=O)c3cc(F)c(F)cc3c21] | 0.03 |
| | N-(8,9-difluoro-6-oxo-1,2,3,4,5,6-hexahydrobenzo[c][1,7]naphthyridin-1- | |
| | yl)-6-(difluoromethyl)-5-fluoro-N-methyl-1H-indole-2-carboxamide | |
| | enantiomer II | |
| 222 | 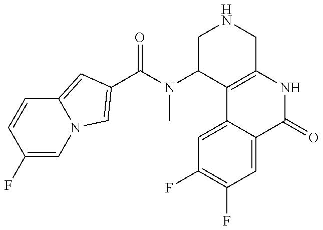 [SMILES: CN(C(=O)c1cc2ccc(F)cn2c1)C1CNCc2[nH]c(=O)c3cc(F)c(F)cc3c21] | 7.07 |
| | N-(8,9-difluoro-6-oxo-1,2,3,4,5,6-hexahydrobenzo[c][1,7]naphthyridin-1- | |
| | yl)-6-fluoro-N-methylindolizine-2-carboxamide | |
| | enantiomer I | |
| 223 | 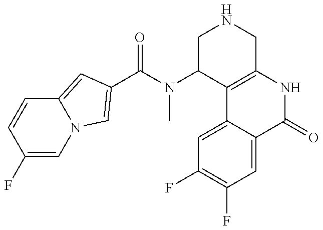 [SMILES: CN(C(=O)c1cc2ccc(F)cn2c1)C1CNCc2[nH]c(=O)c3cc(F)c(F)cc3c21] | 0.03 |
| | N-(8,9-difluoro-6-oxo-1,2,3,4,5,6-hexahydrobenzo[c][1,7]naphthyridin-1- | |
| | yl)-6-fluoro-N-methylindolizine-2-carboxamide | |
| | enantiomer II | |
| 224 | 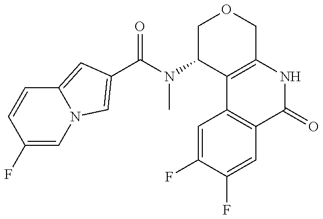 [SMILES: CN(C(=O)c1cc2ccc(F)cn2c1)[C@@H]1COCc2[nH]c(=O)c3cc(F)c(F)cc3c21] | 0.01 |
| | (S)-N-(8,9-difluoro-6-oxo-1,4,5,6-tetrahydro-2H-pyrano[3,4-c]isoquinolin- | |
| | 1-yl)-6-fluoro-N-methylindolizine-2-carboxamide | |
| 225 | 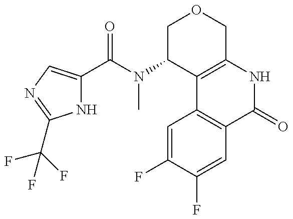 [SMILES: CN(C(=O)c1cnc(C(F)(F)F)[nH]1)[C@@H]1COCc2[nH]c(=O)c3cc(F)c(F)cc3c21] | 13.0 |
| | (S)-N-(8,9-difluoro-6-oxo-1,4,5,6-tetrahydro-2H-pyrano[3,4-c]isoquinolin- | |
| | 1-y1)-N-methyl-2-(trifluoromethyl)-1H-imidazole-5-carboxamide | |
| 226 | 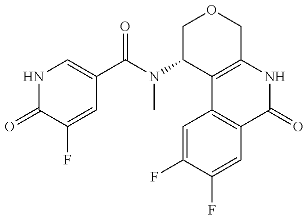 [SMILES: CN(C(=O)c1c[nH]c(=O)c(F)c1)[C@@H]1COCc2[nH]c(=O)c3cc(F)c(F)cc3c21] | 12.5 |
| | (S)-N-(8,9-difluoro-6-oxo-1,4,5,6-tetrahydro-2H-pyrano[3,4-c]isoquinolin- | |
| | 1-yl)-5-fluoro-N-methyl-6-oxo-1,6-dihydropyridine-3-carboxamide | |
| 227 | 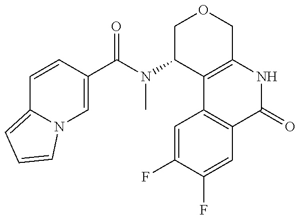 [SMILES: CN(C(=O)c1ccc2cccn2c1)[C@@H]1COCc2[nH]c(=O)c3cc(F)c(F)cc3c21] | 0.02 |
| | (S)-N-(8,9-difluoro-6-oxo-1,4,5,6-tetrahydro-2H-pyrano[3,4-c]isoquinolin- | |
| | 1-yl)-N-methylindolizine-6-carboxamide | |
| 228 | 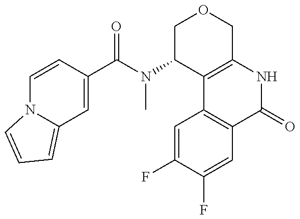 [SMILES: CN(C(=O)c1ccn2cccc2c1)[C@@H]1COCc2[nH]c(=O)c3cc(F)c(F)cc3c21] | 0.06 |
| | (S)-N-(8,9-difluoro-6-oxo-1,4,5,6-tetrahydro-2H-pyrano[3,4-c]isoquinolin- | |
| | 1-yl)-N-methylindolizine-7-carboxamide | |
| 229 | 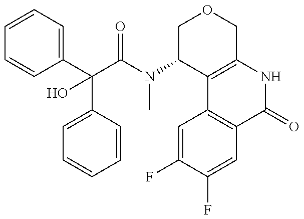 [SMILES: CN(C(=O)C(O)(c1ccccc1)c1ccccc1)[C@@H]1COCc2[nH]c(=O)c3cc(F)c(F)cc3c21] | 8.4 |
| | (S)-N-(8,9-difluoro-6-oxo-1,4,5,6-tetrahydro-2H-pyrano[3,4-c]isoquinolin- | |
| | 1-yl)-2-hydroxy-N-methyl-2,2-diphenylacetamide | |
| 230 | 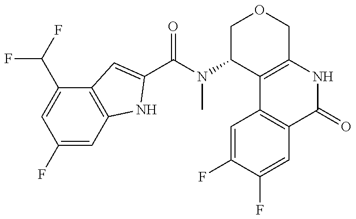 [SMILES: CN(C(=O)c1cc2c(C(F)F)cc(F)cc2[nH]1)[C@@H]1COCc2[nH]c(=O)c3cc(F)c(F)cc3c21] | 0.01 |
| | (S)-N-(8,9-difluoro-6-oxo-1,4,5,6-tetrahydro-2H-pyrano[3,4-c]isoquinolin- | |
| | 1-yl)-4-(difluoromethyl)-6-fluoro-N-methyl-1H-indole-2-carboxamide | |
| 231 | 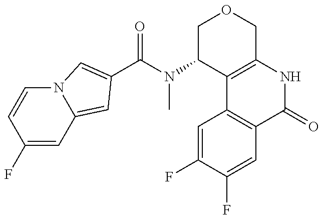 [SMILES: CN(C(=O)c1cc2cc(F)ccn2c1)[C@@H]1COCc2[nH]c(=O)c3cc(F)c(F)cc3c21] | 0.01 |
| | (S)-N-(8,9-difluoro-6-oxo-1,4,5,6-tetrahydro-2H-pyrano[3,4-c]isoquinolin- | |
| | 1-yl)-7-fluoro-N-methylindolizine-2-carboxamide | |
| 232 | 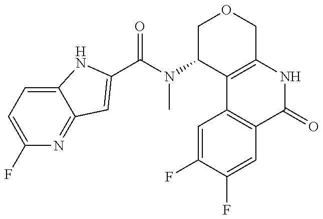 [SMILES: CN(C(=O)c1cc2nc(F)ccc2[nH]1)[C@@H]1COCc2[nH]c(=O)c3cc(F)c(F)cc3c21] | 0.07 |
| | (S)-N-(8,9-difluoro-6-oxo-1,4,5,6-tetrahydro-2H-pyrano[3,4-c]isoquinolin- | |
| | 1-yl)-5-fluoro-N-methyl-1H-pyrrolo[3,2-b]pyridine-2-carboxamide | |
| 233 | 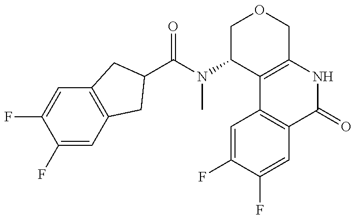 [SMILES: CN(C(=O)C1Cc2cc(F)c(F)cc2C1)[C@@H]1COCc2[nH]c(=O)c3cc(F)c(F)cc3c21] | 0.04 |
| | (S)-N-(8,9-difluoro-6-oxo-1,4,5,6-tetrahydro-2H-pyrano[3,4-c]isoquinolin- | |
| | 1-yl)-5,6-difluoro-N-methyl-2,3-dihydro-1H-indene-2-carboxamide | |
| 234 | 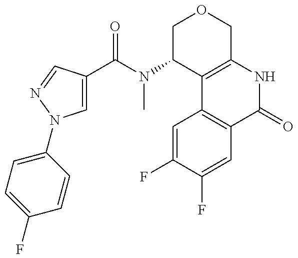 [SMILES: CN(C(=O)c1cnn(-c2ccc(F)cc2)c1)[C@@H]1COCc2[nH]c(=O)c3cc(F)c(F)cc3c21] | 10.5 |
| | (S)-N-(8,9-difluoro-6-oxo-1,4,5,6-tetrahydro-2H-pyrano[3,4-c]isoquinolin- | |
| | 1-yl)-1-(4-fluorophenyl)-N-methyl-1H-pyrazole-4-carboxamide | |
| 236 | 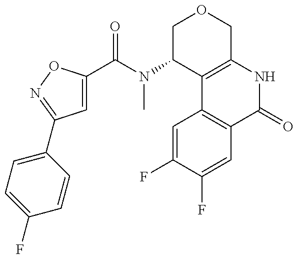 [SMILES: CN(C(=O)c1cc(-c2ccc(F)cc2)no1)[C@@H]1COCc2[nH]c(=O)c3cc(F)c(F)cc3c21] | 15.1 |
| | (S)-N-(8,9-difluoro-6-oxo-1,4,5,6-tetrahydro-2H-pyrano[3,4-c]isoquinolin- | |
| | 1-yl)-3-(4-fluorophenyl)-N-methylisoxazole-5-carboxamide | |
| 237 | 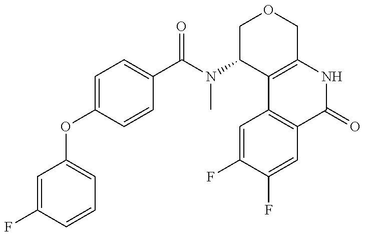 [SMILES: CN(C(=O)c1ccc(Oc2cccc(F)c2)cc1)[C@@H]1COCc2[nH]c(=O)c3cc(F)c(F)cc3c21] | 6.9 |
| | (S)-N-(8,9-difluoro-6-oxo-1,4,5,6-tetrahydro-2H-pyrano[3,4-c]isoquinolin- | |
| | 1-yl)-4-(3-fluorophenoxy)-N-methylbenzamide | |
| 238 | 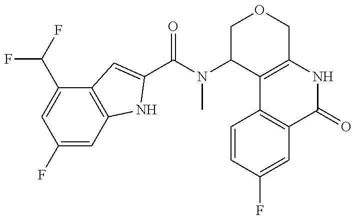 [SMILES: CN(C(=O)c1cc2c(C(F)F)cc(F)cc2[nH]1)C1COCc2[nH]c(=O)c3cc(F)ccc3c21] | 1.4 |
| | 4-(difluoromethyl)-6-fluoro-N-(8-fluoro-6-oxo-1,4,5,6-tetrahydro-2H- | |
| | pyrano[3,4-c]isoquinolin-1-yl)-N-methyl-1H-indole-2-carboxamide | |
| | enantiomer I | |
| 239 | 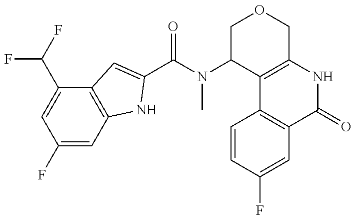 [SMILES: CN(C(=O)c1cc2c(C(F)F)cc(F)cc2[nH]1)C1COCc2[nH]c(=O)c3cc(F)ccc3c21] | 0.01 |
| | 4-(difluoromethyl)-6-fluoro-N-(8-fluoro-6-oxo-1,4,5,6-tetrahydro-2H- | |
| | pyrano[3,4-c]isoquinolin-1-yl)-N-methyl-1H-indole-2-carboxamide | |
| | enantiomer II | |
| 240 | 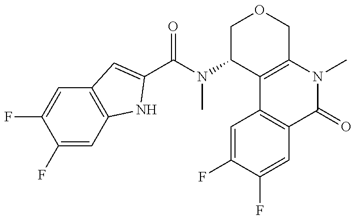 [SMILES: CN(C(=O)c1cc2cc(F)c(F)cc2[nH]1)[C@@H]1COCc2c1c1cc(F)c(F)cc1c(=O)n2C] | 0.01 |
| | (S)-N-(8,9-difluoro-5-methyl-6-oxo-1,4,5,6-tetrahydro-2H-pyrano[3,4- | |
| | c]isoquinolin-1-yl)-5,6-difluoro-N-methyl-1H-indole-2-carboxamide | |
| 241 | 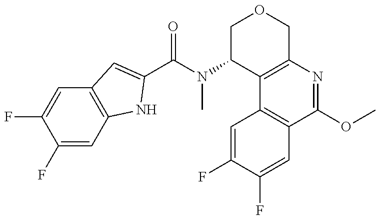 [SMILES: COc1nc2c(c3cc(F)c(F)cc13)[C@H](N(C)C(=O)c1cc3cc(F)c(F)cc3[nH]1)COC2] | 0.03 |
| | (S)-N-(8,9-difluoro-6-methoxy-1,4-dihydro-2H-pyrano[3,4-c]isoquinolin-1- | |
| | yl)-5,6-difluoro-N-methyl-1H-indole-2-carboxamide | |
| 242 | 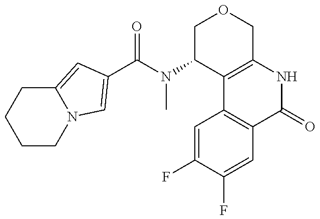 [SMILES: CN(C(=O)c1cc2n(c1)CCCC2)[C@@H]1COCc2[nH]c(=O)c3cc(F)c(F)cc3c21] | 0.11 |
| | (S)-N-(8,9-difluoro-6-oxo-1,4,5,6-tetrahydro-2H-pyrano[3,4-c]isoquinolin- | |
| | 1-yl)-N-methyl-5,6,7,8-tetrahydroindolizine-2-carboxamide | |
| 244 | 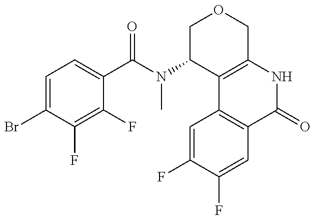 [SMILES: CN(C(=O)c1ccc(Br)c(F)c1F)[C@@H]1COCc2[nH]c(=O)c3cc(F)c(F)cc3c21] | 0.08 |
| | (S)-4-bromo-N-(8,9-difluoro-6-oxo-1,4,5,6-tetrahydro-2H-pyrano[3,4- | |
| | c]isoquinolin-1-yl)-2,3-difluoro-N-methylbenzamide | |
| 245 | 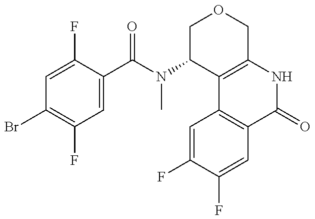 [SMILES: CN(C(=O)c1cc(F)c(Br)cc1F)[C@@H]1COCc2[nH]c(=O)c3cc(F)c(F)cc3c21] | 0.17 |
| | (S)-4-bromo-N-(8,9-difluoro-6-oxo-1,4,5,6-tetrahydro-2H-pyrano[3,4- | |
| | c]isoquinolin-1-yl)-2,5-difluoro-N-methylbenzamide | |
| 246 | 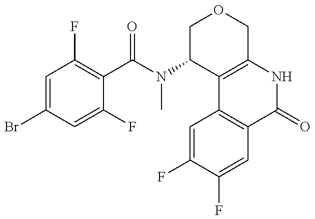 [SMILES: CN(C(=O)c1c(F)cc(Br)cc1F)[C@@H]1COCc2[nH]c(=O)c3cc(F)c(F)cc3c21] | 1.96 |
| | (S)-4-bromo-N-(8,9-difluoro-6-oxo-1,4,5,6-tetrahydro-2H-pyrano[3,4- | |
| | c]isoquinolin-1-yl)-2,6-difluoro-N-methylbenzamide | |
| 247 | 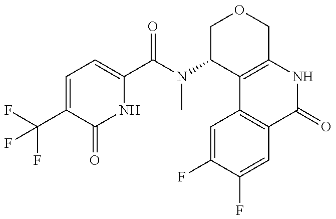 [SMILES: CN(C(=O)c1ccc(C(F)(F)F)c(=O)[nH]1)[C@@H]1COCc2[nH]c(=O)c3cc(F)c(F)cc3c21] | 3.87 |
| | (S)-N-(8,9-difluoro-6-oxo-1,4,5,6-tetrahydro-2H-pyrano[3,4-c]isoquinolin- | |
| | 1-yl)-N-methyl-6-oxo-5-(trifluoromethyl)-1,6-dihydropyridine-2- | |
| | carboxamide | |
| 248 | 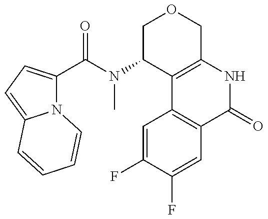 [SMILES: CN(C(=O)c1ccc2ccccn12)[C@@H]1COCc2[nH]c(=O)c3cc(F)c(F)cc3c21] | 5.38 |
| | (S)-N-(8,9-difluoro-6-oxo-1,4,5,6-tetrahydro-2H-pyrano[3,4-c]isoquinolin- | |
| | 1-yl)-N-methylindolizine-3-carboxamide | |
| 249 | 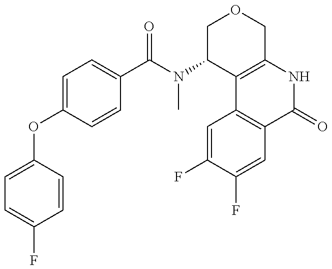 [SMILES: CN(C(=O)c1ccc(Oc2ccc(F)cc2)cc1)[C@@H]1COCc2[nH]c(=O)c3cc(F)c(F)cc3c21] | 4.6 |
| | (S)-N-(8,9-difluoro-6-oxo-1,4,5,6-tetrahydro-2H-pyrano[3,4-c]isoquinolin- | |
| | 1-yl)-4-(4-fluorophenoxy)-N-methylbenzamide | |
| 250 | 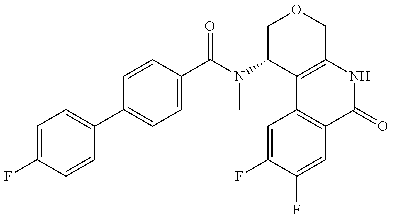 [SMILES: CN(C(=O)c1ccc(-c2ccc(F)cc2)cc1)[C@@H]1COCc2[nH]c(=O)c3cc(F)c(F)cc3c21] | 8.1 |
| | (S)-N-(8,9-difluoro-6-oxo-1,4,5,6-tetrahydro-2H-pyrano[3,4-c]isoquinolin- | |
| | 1-yl)-4'-fluoro-N-methyl-[1,l'-biphenyl]-4-carboxamide | |
| 251 | 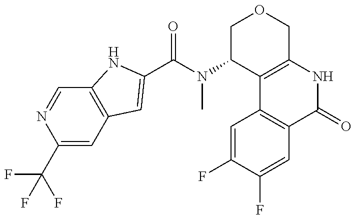 [SMILES: CN(C(=O)c1cc2cc(C(F)(F)F)ncc2[nH]1)[C@@H]1COCc2[nH]c(=O)c3cc(F)c(F)cc3c21] | 0.23 |
| | (S)-N-(8,9-difluoro-6-oxo-1,4,5,6-tetrahydro-2H-pyrano[3,4-c]isoquinolin- | |
| | 1-yl)-N-methyl-5-(trifluoromethyl)-1H-pyrrolo[2,3-c]pyridine-2- | |
| | carboxamide | |
| 252 | 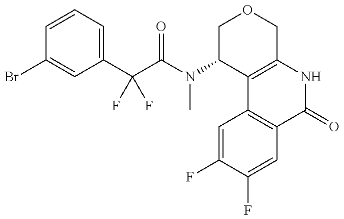 [SMILES: CN(C(=O)C(F)(F)c1cccc(Br)c1)[C@@H]1COCc2[nH]c(=O)c3cc(F)c(F)cc3c21] | 7.7 |
| | (S)-2-(3-bromophenyl)-N-(8,9-difluoro-6-oxo-1,4,5,6-tetrahydro-2H- | |
| | pyrano[3,4-c]isoquinolin-1-yl)-2,2-difluoro-N-methylacetamide | |
| 253 | 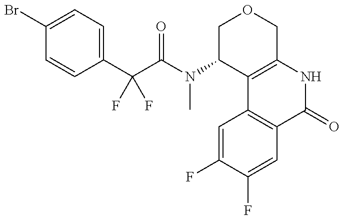 [SMILES: CN(C(=O)C(F)(F)c1ccc(Br)cc1)[C@@H]1COCc2[nH]c(=O)c3cc(F)c(F)cc3c21] | 8.0 |
| | (S)-2-(4-bromophenyl)-N-(8,9-difluoro-6-oxo-1,4,5,6-tetrahydro-2H- | |
| | pyrano[3,4-c]isoquinolin-1-y1)-2,2-difluoro-N-methylacetamide | |
| 254 | 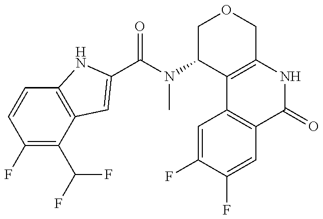 [SMILES: CN(C(=O)c1cc2c(C(F)F)c(F)ccc2[nH]1)[C@@H]1COCc2[nH]c(=O)c3cc(F)c(F)cc3c21] | 0.01 |
| | (S)-N-(8,9-difluoro-6-oxo-1,4,5,6-tetrahydro-2H-pyrano[3,4-c]isoquinolin- | |
| | 1-y1)-4-(difluoromethyl)-5-fluoro-N-methyl-1H-indole-2-carboxamide | |
| 255 | 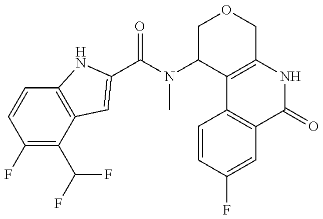 [SMILES: CN(C(=O)c1cc2c(C(F)F)c(F)ccc2[nH]1)C1COCc2[nH]c(=O)c3cc(F)ccc3c21] | 0.68 |
| | 4-(difluoromethyl)-5-fluoro-N-(8-fluoro-6-oxo-1,4,5,6-tetrahydro-2H- | |
| | pyrano[3,4-c]isoquinolin-1-y1)-N-methyl-1H-indole-2-carboxamide | |
| | enantiomer I | |
| 256 | 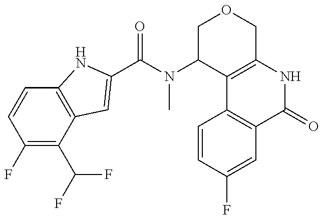 [SMILES: CN(C(=O)c1cc2c(C(F)F)c(F)ccc2[nH]1)C1COCc2[nH]c(=O)c3cc(F)ccc3c21] | 0.01 |
| | 4-(difluoromethyl)-5-fluoro-N-(8-fluoro-6-oxo-1,4,5,6-tetrahydro-2H- | |
| | pyrano[3,4-c]isoquinolin-1-yl)-N-methyl-1H-indole-2-carboxamide | |
| | enantiomer II | |
| 257 | 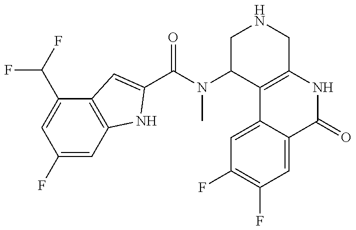 [SMILES: CN(C(=O)c1cc2c(C(F)F)cc(F)cc2[nH]1)C1CNCc2[nH]c(=O)c3cc(F)c(F)cc3c21] | 2.2 |
| | N-(8,9-difluoro-6-oxo-1,2,3,4,5,6-hexahydrobenzo[c][1,7]naphthyridin-1- | |
| | y1)-4-(difluoromethyl)-6-fluoro-N-methyl-1H-indole-2-carboxamide | |
| | enantiomer I | |
| 258 | 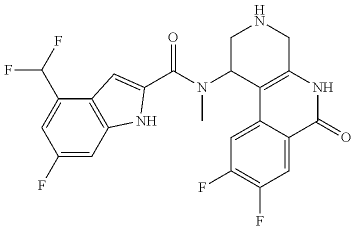 [SMILES: CN(C(=O)c1cc2c(C(F)F)cc(F)cc2[nH]1)C1CNCc2[nH]c(=O)c3cc(F)c(F)cc3c21] | 0.02 |
| | N-(8,9-difluoro-6-oxo-1,2,3,4,5,6-hexahydrobenzo[c][1,7]naphthyridin-1- | |
| | yl)-4-(difluoromethyl)-6-fluoro-N-methyl-1H-indole-2-carboxamide | |
| | enantiomer II | |
| 259 | 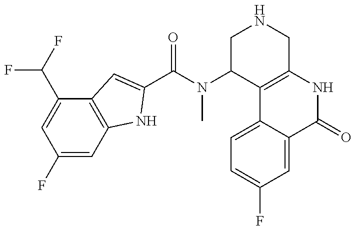 [SMILES: CN(C(=O)c1cc2c(C(F)F)cc(F)cc2[nH]1)C1CNCc2[nH]c(=O)c3cc(F)ccc3c21] | 5.1 |
| | 4-(difluoromethyl)-6-fluoro-N-(8-fluoro-6-oxo-1,2,3,4,5,6- | |
| | hexahydrobenzo[c][1,7]naphthyridin-1-yl)-N-methyl-1H-indole-2- | |
| | carboxamide | |
| | enantiomer I | |
| 260 | 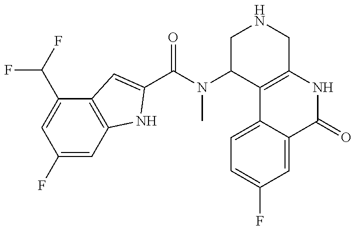 [SMILES: CN(C(=O)c1cc2c(C(F)F)cc(F)cc2[nH]1)C1CNCc2[nH]c(=O)c3cc(F)ccc3c21] | 0.06 |
| | 4-(difluoromethyl)-6-fluoro-N-(8-fluoro-6-oxo-1,2,3,4,5,6- | |
| | hexahydrobenzo[c][1,7]naphthyridin-1-yl)-N-methyl-1H-indole-2- | |
| | carboxamide | |
| | enantiomer II | |
| 261 | 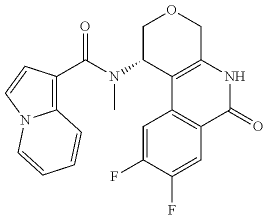 [SMILES: CN(C(=O)c1ccn2ccccc12)[C@@H]1COCc2[nH]c(=O)c3cc(F)c(F)cc3c21] | 6.40 |
| | (S)-N-(8,9-difluoro-6-oxo-1,4,5,6-tetrahydro-2H-pyrano[3,4-c]isoquinolin- | |
| | 1-yl)-N-methylindolizine-1-carboxamide | |
| 262 | 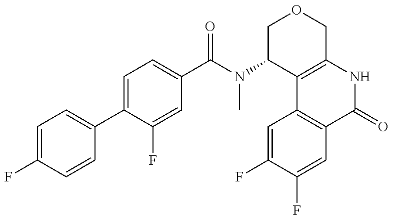 [SMILES: CN(C(=O)c1ccc(-c2ccc(F)cc2)c(F)c1)[C@@H]1COCc2[nH]c(=O)c3cc(F)c(F)cc3c21] | 17.2 |
| | (S)-N-(8,9-difluoro-6-oxo-1,4,5,6-tetrahydro-2H-pyrano[3,4-c]isoquinolin- | |
| | 1-yl)-2,4'-difluoro-N-methyl-[1,l'-biphenyl]-4-carboxamide | |
| 263 | 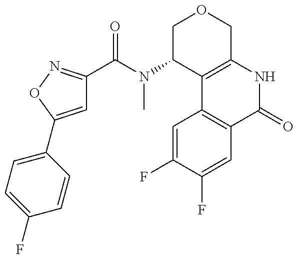 [SMILES: CN(C(=O)c1cc(-c2ccc(F)cc2)on1)[C@@H]1COCc2[nH]c(=O)c3cc(F)c(F)cc3c21] | 14.7 |
| | (S)-N-(8,9-difluoro-6-oxo-1,4,5,6-tetrahydro-2H-pyrano[3,4-c]isoquinolin- | |
| | 1-yl)-5-(4-fluorophenyl)-N-methylisoxazole-3-carboxamide | |
| 264 | 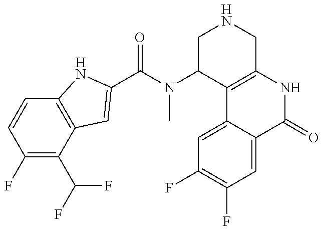 [SMILES: CN(C(=O)c1cc2c(C(F)F)c(F)ccc2[nH]1)C1CNCc2[nH]c(=O)c3cc(F)c(F)cc3c21] | 8.23 |
| | N-(8,9-difluoro-6-oxo-1,2,3,4,5,6-hexahydrobenzo[c][1,7]naphthyridin-1- | |
| | yl)-4-(difluoromethyl)-5-fluoro-N-methyl-1H-indole-2-carboxamide | |
| | enantiomer I | |
| 265 | 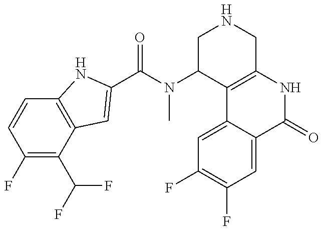 [SMILES: CN(C(=O)c1cc2c(C(F)F)c(F)ccc2[nH]1)C1CNCc2[nH]c(=O)c3cc(F)c(F)cc3c21] | 0.02 |
| | N-(8,9-difluoro-6-oxo-1,2,3,4,5,6-hexahydrobenzo[c][1,7]naphthyridin-1- | |
| | yl)-4-(difluoromethyl)-5-fluoro-N-methyl-1H-indole-2-carboxamide | |
| | enantiomer II | |
| 266 | 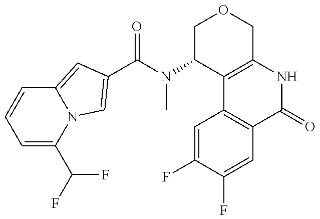 [SMILES: CN(C(=O)c1cc2cccc(C(F)F)n2c1)[C@@H]1COCc2[nH]c(=O)c3cc(F)c(F)cc3c21] | 0.02 |
| | (S)-N-(8,9-difluoro-6-oxo-1,4,5,6-tetrahydro-2H-pyrano[3,4-c]isoquinolin- | |
| | 1-yl)-5-(difluoromethyl)-N-methylindolizine-2-carboxamide | |
| 268 | 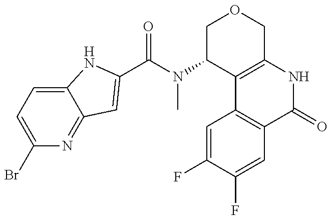 [SMILES: CN(C(=O)c1cc2nc(Br)ccc2[nH]1)[C@@H]1COCc2[nH]c(=O)c3cc(F)c(F)cc3c21] | 1.8 |
| | (S)-5-bromo-N-(8,9-difluoro-6-oxo-1,4,5,6-tetrahydro-2H-pyrano[3,4- | |
| | c]isoquinolin-1-yl)-N-methyl-1H-pyrrolo[3,2-b]pyridine-2-carboxamide | |
| 269 | 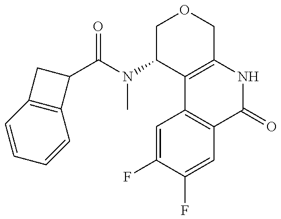 [SMILES: CN(C(=O)C1Cc2ccccc21)[C@@H]1COCc2[nH]c(=O)c3cc(F)c(F)cc3c21] | 18.6 |
| | N-((S)-8,9-difluoro-6-oxo-1,4,5,6-tetrahydro-2H-pyrano[3,4-c]isoquinolin- | |
| | 1-y1)-N-methylbicyclo[4.2.0]octa-1(6),2,4-triene-7-carboxamide | |
| 271 | 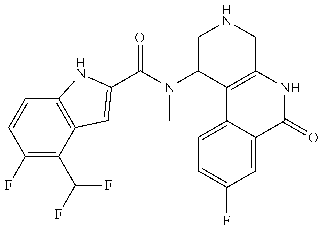 [SMILES: CN(C(=O)c1cc2c(C(F)F)c(F)ccc2[nH]1)C1CNCc2[nH]c(=O)c3cc(F)ccc3c21] | 6.7 |
| | 4-(difluoromethyl)-5-fluoro-N-(8-fluoro-6-oxo-1,2,3,4,5,6- | |
| | hexahydrobenzo[c][1,7]naphthyridin-1-y1)-N-methyl-1H-indole-2- | |
| | carboxamide | |
| | enantiomer I | |
| 272 | 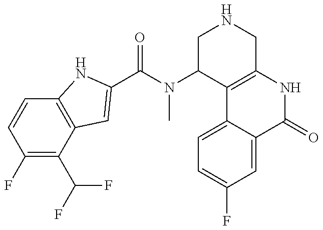 [SMILES: CN(C(=O)c1cc2c(C(F)F)c(F)ccc2[nH]1)C1CNCc2[nH]c(=O)c3cc(F)ccc3c21] | 0.07 |
| | 4-(difluoromethyl)-5-fluoro-N-(8-fluoro-6-oxo-1,2,3,4,5,6- | |
| | hexahydrobenzo[c][1,7]naphthyridin-1-yl)-N-methyl-1H-indole-2- | |
| | carboxamide | |
| | enantiomer II | |
| 275 | 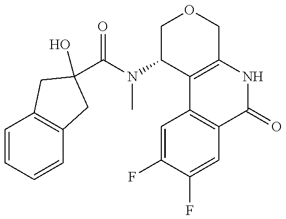 [SMILES: CN(C(=O)C1(O)Cc2ccccc2C1)[C@@H]1COCc2[nH]c(=O)c3cc(F)c(F)cc3c21] | 3.84 |
| | (S)-N-(8,9-difluoro-6-oxo-1,4,5,6-tetrahydro-2H-pyrano[3,4-c]isoquinolin- | |
| | 1-yl)-2-hydroxy-N-methyl-2,3-dihydro-1H-indene-2-carboxamide | |
| 276 | 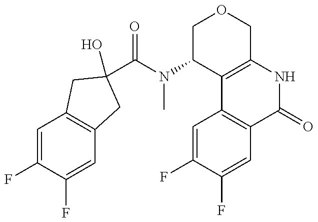 [SMILES: CN(C(=O)C1(O)Cc2cc(F)c(F)cc2C1)[C@@H]1COCc2[nH]c(=O)c3cc(F)c(F)cc3c21] | 1.95 |
| | (S)-N-(8,9-difluoro-6-oxo-1,4,5,6-tetrahydro-2H-pyrano[3,4-c]isoquinolin- | |
| | 1-yl)-5,6-difluoro-2-hydroxy-N-methyl-2,3-dihydro-1H-indene-2- | |
| | carboxamide | |
| 277 | 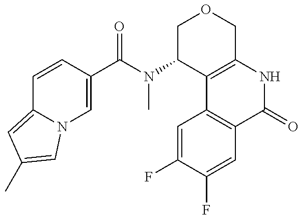 [SMILES: Cc1cc2ccc(C(=O)N(C)[C@@H]3COCc4[nH]c(=O)c5cc(F)c(F)cc5c43)cn2c1] | 0.42 |
| | (S)-N-(8,9-difluoro-6-oxo-1,4,5,6-tetrahydro-2H-pyrano[3,4-c]isoquinolin- | |
| | 1-yl)-N,2-dimethylindolizine-6-carboxamide | |
| 278 | 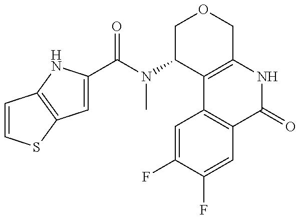 [SMILES: CN(C(=O)c1cc2sccc2[nH]1)[C@@H]1COCc2[nH]c(=O)c3cc(F)c(F)cc3c21] | 0.02 |
| | (S)-N-(8,9-difluoro-6-oxo-1,4,5,6-tetrahydro-2H-pyrano[3,4-c]isoquinolin- | |
| | 1-yl)-N-methyl-4H-thieno[3,2-b]pyrrole-5-carboxamide | |
| 279 | 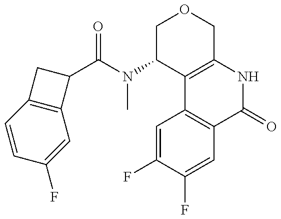 [SMILES: CN(C(=O)C1Cc2ccc(F)cc21)[C@@H]1COCc2[nH]c(=O)c3cc(F)c(F)cc3c21] | 9.5 |
| | N-((S)-8,9-difluoro-6-oxo-1,4,5,6-tetrahydro-2H-pyrano[3,4-c]isoquinolin- | |
| | 1-yl)-4-fluoro-N-methylbicyclo[4.2.0]octa-1(6),2,4-triene-7-carboxamide | |
| 280 | 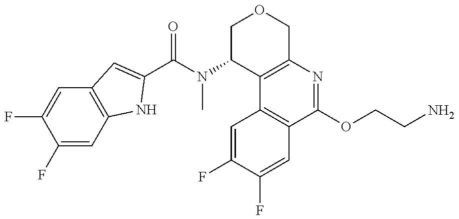 [SMILES: CN(C(=O)c1cc2cc(F)c(F)cc2[nH]1)[C@@H]1COCc2nc(OCCN)c3cc(F)c(F)cc3c21] | 0.02 |
| | (S)-N-(6-(2-aminoethoxy)-8,9-difluoro-1,4-dihydro-2H-pyrano[3,4- | |
| | c]isoquinolin-1-yl)-5,6-difluoro-N-methyl-1H-indole-2-carboxamide | |
| 282 | 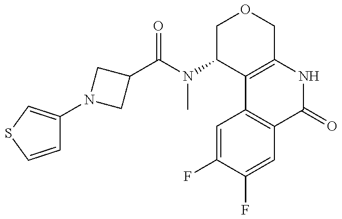 [SMILES: CN(C(=O)C1CN(c2ccsc2)C1)[C@@H]1COCc2[nH]c(=O)c3cc(F)c(F)cc3c21] | 0.29 |
| | (S)-N-(8,9-difluoro-6-oxo-1,4,5,6-tetrahydro-2H-pyrano[3,4-c]isoquinolin- | |
| | 1-yl)-N-methyl-1-(thiophen-3-yl)azetidine-3-carboxamide | |
| 283 | 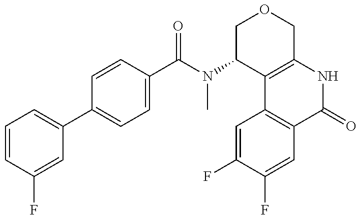 [SMILES: CN(C(=O)c1ccc(-c2cccc(F)c2)cc1)[C@@H]1COCc2[nH]c(=O)c3cc(F)c(F)cc3c21] | 4.7 |
| | (S)-N-(8,9-difluoro-6-oxo-1,4,5,6-tetrahydro-2H-pyrano[3,4-c]isoquinolin- | |
| | 1-yl)-3′-fluoro-N-methyl-[1,l′-biphenyl]-4-carboxamide | |
| 284 | 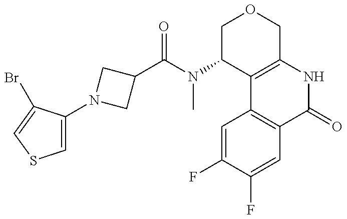 [SMILES: CN(C(=O)C1CN(c2cscc2Br)C1)[C@@H]1COCc2[nH]c(=O)c3cc(F)c(F)cc3c21] | 0.05 |
| | (S)-1-(4-bromothiophen-3-yl)-N-(8,9-difluoro-6-oxo-1,4,5,6-tetrahydro- | |
| | 2H- pyrano[3,4-c]isoquinolin-1-yl)-N-methylazetidine-3-carboxamide | |
| 285 | 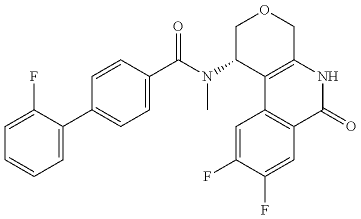 [SMILES: CN(C(=O)c1ccc(-c2ccccc2F)cc1)[C@@H]1COCc2[nH]c(=O)c3cc(F)c(F)cc3c21] | 19.7 |
| | (S)-N-(8,9-difluoro-6-oxo-1,4,5,6-tetrahydro-2H-pyrano[3,4-c]isoquinolin- | |
| | 1-y1)-2′-fluoro-N-methyl-[1,l′-biphenyl]-4-carboxamide | |
| 286 | 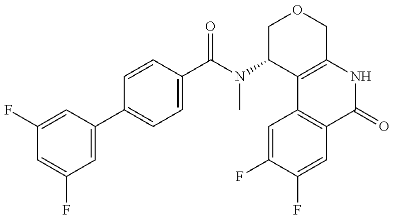 [SMILES: CN(C(=O)c1ccc(-c2cc(F)cc(F)c2)cc1)[C@@H]1COCc2[nH]c(=O)c3cc(F)c(F)cc3c21] | 12.9 |
| | (S)-N-(8,9-difluoro-6-oxo-1,4,5,6-tetrahydro-2H-pyrano[3,4-c]isoquinolin- | |
| | 1-yl)-3′,5′-difluoro-N-methyl-[1,l′-biphenyl]-4-carboxamide | |
| 287 | 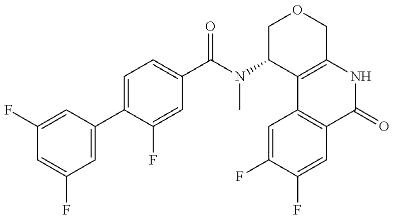 [SMILES: CN(C(=O)c1ccc(-c2cc(F)cc(F)c2)c(F)c1)[C@@H]1COCc2[nH]c(=O)c3cc(F)c(F)cc3c21] | 12.7 |
| | (S)-N-(8,9-difluoro-6-oxo-1,4,5,6-tetrahydro-2H-pyrano[3,4-c]isoquinolin- | |
| | 1-yl)-2,3',5'-trifluoro-N-methyl-[1,l'-biphenyl]-4-carboxamide | |
| 288 | 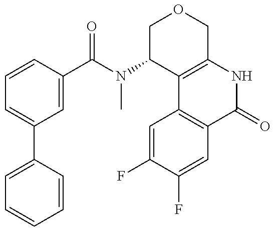 [SMILES: CN(C(=O)c1cccc(-c2ccccc2)c1)[C@@H]1COCc2[nH]c(=O)c3cc(F)c(F)cc3c21] | 11.3 |
| | (S)-N-(8,9-difluoro-6-oxo-1,4,5,6-tetrahydro-2H-pyrano[3,4-c]isoquinolin- | |
| | 1-y1)-N-methyl-[1,l′-biphenyl]-3-carboxamide | |
| 289 | 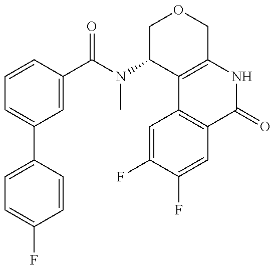 [SMILES: CN(C(=O)c1cccc(-c2ccc(F)cc2)c1)[C@@H]1COCc2[nH]c(=O)c3cc(F)c(F)cc3c21] | 6.4 |
| | (S)-N-(8,9-difluoro-6-oxo-1,4,5,6-tetrahydro-2H-pyrano[3,4-c]isoquinolin- | |
| | 1-y1)-4′-fluoro-N-methyl-[1,l′-biphenyl]-3-carboxamide | |
| 290 | 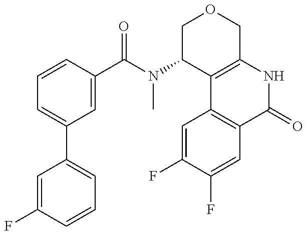 [SMILES: CN(C(=O)c1cccc(-c2cccc(F)c2)c1)[C@@H]1COCc2[nH]c(=O)c3cc(F)c(F)cc3c21] | 2.3 |
| | (S)-N-(8,9-difluoro-6-oxo-1,4,5,6-tetrahydro-2H-pyrano[3,4-c]isoquinolin- | |
| | 1-yl)-3'-fluoro-N-methyl-[1,l'-biphenyl]-3-carboxamide | |
| 291 | 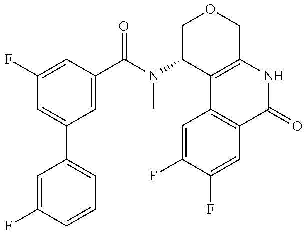 [SMILES: CN(C(=O)c1cc(F)cc(-c2cccc(F)c2)c1)[C@@H]1COCc2[nH]c(=O)c3cc(F)c(F)cc3c21] | 10.1 |
| | (S)-N-(8,9-difluoro-6-oxo-1,4,5,6-tetrahydro-2H-pyrano[3,4-c]isoquinolin- | |
| | 1-yl)-3′,5-difluoro-N-methyl-[1,l'-biphenyl]-3-carboxamide | |
| 292 | 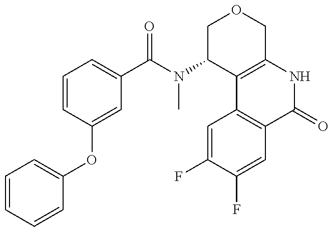 [SMILES: CN(C(=O)c1cccc(Oc2ccccc2)c1)[C@@H]1COCc2[nH]c(=O)c3cc(F)c(F)cc3c21] | 11.2 |
| | (S)-N-(8,9-difluoro-6-oxo-1,4,5,6-tetrahydro-2H-pyrano[3,4-c]isoquinolin- | |
| | 1-yl)-N-methyl-3-phenoxybenzamide | |
| 293 | 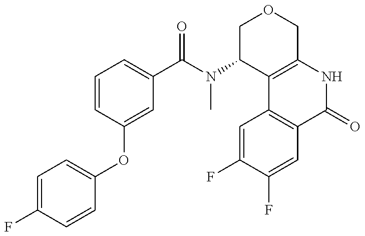 [SMILES: CN(C(=O)c1cccc(Oc2ccc(F)cc2)c1)[C@@H]1COCc2[nH]c(=O)c3cc(F)c(F)cc3c21] | 7.6 |
| | (S)-N-(8,9-difluoro-6-oxo-1,4,5,6-tetrahydro-2H-pyrano[3,4-c]isoquinolin- | |
| | 1-y1)-3-(4-fluorophenoxy)-N-methylbenzamide | |
| 294 | 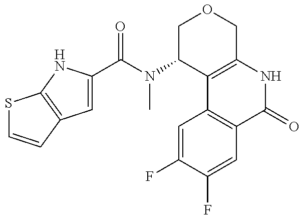 [SMILES: CN(C(=O)c1cc2ccsc2[nH]1)[C@@H]1COCc2[nH]c(=O)c3cc(F)c(F)cc3c21] | 0.05 |
| | (S)-N-(8,9-difluoro-6-oxo-1,4,5,6-tetrahydro-2H-pyrano[3,4-c]isoquinolin- | |
| | 1-y1)-N-methyl-6H-thieno[2,3-b]pyrrole-5-carboxamide | |
| 295 | 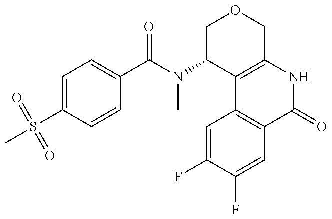 [SMILES: CN(C(=O)c1ccc(S(C)(=O)=O)cc1)[C@@H]1COCc2[nH]c(=O)c3cc(F)c(F)cc3c21] | 0.35 |
| | (S)-N-(8,9-difluoro-6-oxo-1,4,5,6-tetrahydro-2H-pyrano[3,4-c]isoquinolin- | |
| | 1-yl)-N-methyl-4-(methylsulfonyl)benzamide | |
| 296 | 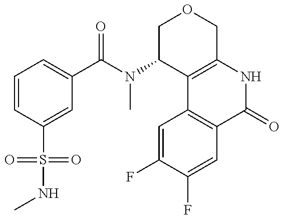 [SMILES: CNS(=O)(=O)c1cccc(C(=O)N(C)[C@@H]2COCc3[nH]c(=O)c4cc(F)c(F)cc4c32)c1] | 0.89 |
| | (S)-N-(8,9-difluoro-6-oxo-1,4,5,6-tetrahydro-2H-pyrano[3,4-c]isoquinolin- | |
| | 1-yl)-N-methyl-3-(N-methylsulfamoyl)benzamide | |
| 297 | 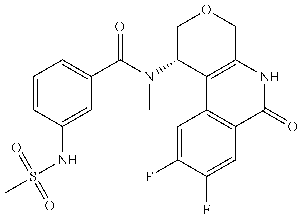 [SMILES: CN(C(=O)c1cccc(NS(C)(=O)=O)c1)[C@@H]1COCc2[nH]c(=O)c3cc(F)c(F)cc3c21] | 21 |
| | (S)-N-(8,9-difluoro-6-oxo-1,4,5,6-tetrahydro-2H-pyrano[3,4-c]isoquinolin- | |
| | 1-y1)-N-methyl-3-(methylsulfonamido)benzamide | |
| 298 | 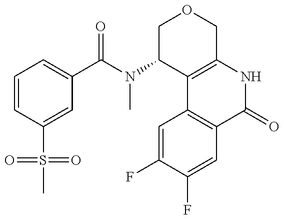 [SMILES: CN(C(=O)c1cccc(S(C)(=O)=O)c1)[C@@H]1COCc2[nH]c(=O)c3cc(F)c(F)cc3c21] | 9.00 |
| | (S)-N-(8,9-difluoro-6-oxo-1,4,5,6-tetrahydro-2H-pyrano[3,4-c]isoquinolin- | |
| | 1-yl)-N-methyl-3-(methylsulfonyl)benzamide | |
| 299 | 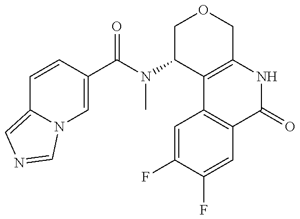 [SMILES: CN(C(=O)c1ccc2cncn2c1)[C@@H]1COCc2[nH]c(=O)c3cc(F)c(F)cc3c21] | 0.36 |
| | (S)-N-(8,9-difluoro-6-oxo-1,4,5,6-tetrahydro-2H-pyrano[3,4-c]isoquinolin- | |
| | 1-y1)-N-methylimidazo[1,5-a]pyridine-6-carboxamide | |
| 300 | 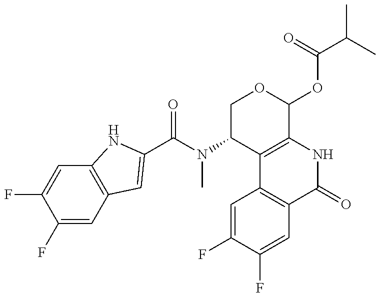 [SMILES: CC(C)C(=O)OC1OC[C@@H](N(C)C(=O)c2cc3cc(F)c(F)cc3[nH]2)c2c1[nH]c(=O)c1cc(F)c(F)cc21] | 0.01 |
| | (1S)-1-(5,6-difluoro-N-methyl-1H-indole-2-carboxamido)-8,9-difluoro-6- | |
| | oxo-1,4,5,6-tetrahydro-2H-pyrano[3,4-c]isoquinolin-4-yl isobutyrate | |
| | ~6:1 ratio of epimers at anomeric carbon | |
| 301 | 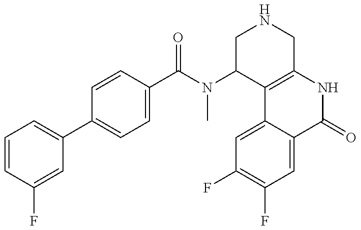 [SMILES: CN(C(=O)c1ccc(-c2cccc(F)c2)cc1)C1CNCc2[nH]c(=O)c3cc(F)c(F)cc3c21] | 24.5 |
| | N-(8,9-difluoro-6-oxo-1,2,3,4,5,6-hexahydrobenzo[c][1,7]naphthyridin-1- | |
| | yl)-3′-fluoro-N-methyl-[1,l′-biphenyl]-4-carboxamide | |
| | enantiomer I | |
| 302 | 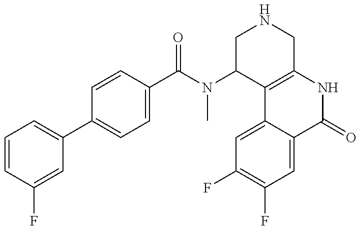 [SMILES: CN(C(=O)c1ccc(-c2cccc(F)c2)cc1)C1CNCc2[nH]c(=O)c3cc(F)c(F)cc3c21] | 1.06 |
| | N-(8,9-difluoro-6-oxo-1,2,3,4,5,6-hexahydrobenzo[c][1,7]naphthyridin-1- | |
| | y1)-3'-fluoro-N-methyl-[1,l'-biphenyl]-4-carboxamide | |
| | enantiomer II | |
| 303 | 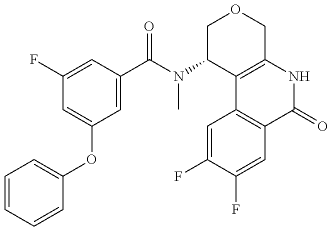 [SMILES: CN(C(=O)c1cc(F)cc(Oc2ccccc2)c1)[C@@H]1COCc2[nH]c(=O)c3cc(F)c(F)cc3c21] | 6.7 |
| | (S)-N-(8,9-difluoro-6-oxo-1,4,5,6-tetrahydro-2H-pyrano[3,4-c]isoquinolin- | |
| | 1-yl)-3-fluoro-N-methyl-5-phenoxybenzamide | |
| 304 | 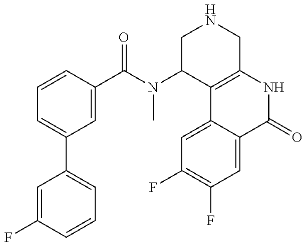 [SMILES: CN(C(=O)c1cccc(-c2cccc(F)c2)c1)C1CNCc2[nH]c(=O)c3cc(F)c(F)cc3c21] | 20 |
| | N-(8,9-difluoro-6-oxo-1,2,3,4,5,6-hexahydrobenzo[c][1,7]naphthyridin-1- | |
| | yl)-3'-fluoro-N-methyl-[1,l'-biphenyl]-3-carboxamide | |
| | enantiomer I | |
| 306 | 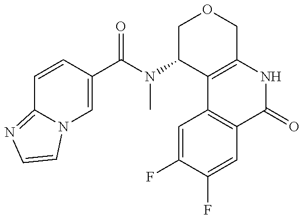 [SMILES: CN(C(=O)c1ccc2nccn2c1)[C@@H]1COCc2[nH]c(=O)c3cc(F)c(F)cc3c21] | 0.42 |
| | (S)-N-(8,9-difluoro-6-oxo-1,4,5,6-tetrahydro-2H-pyrano[3,4-c]isoquinolin- | |
| | 1-y1)-N-methylimidazo[1,2-a]pyridine-6-carboxamide | |
| 307 | 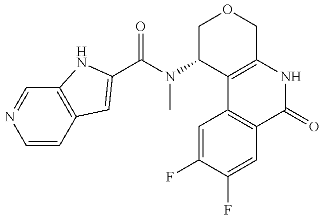 [SMILES: CN(C(=O)c1cc2ccncc2[nH]1)[C@@H]1COCc2[nH]c(=O)c3cc(F)c(F)cc3c21] | 0.20 |
| | (S)-N-(8,9-difluoro-6-oxo-1,4,5,6-tetrahydro-2H-pyrano[3,4-c]isoquinolin- | |
| | 1-y1)-N-methyl-1H-pyrrolo[2,3-c]pyridine-2-carboxamide | |
| 308 | 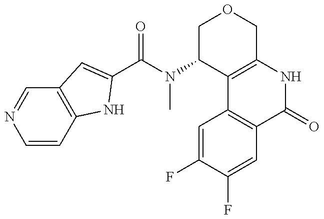 [SMILES: CN(C(=O)c1cc2cnccc2[nH]1)[C@@H]1COCc2[nH]c(=O)c3cc(F)c(F)cc3c21] | 0.61 |
| | (S)-N-(8,9-difluoro-6-oxo-1,4,5,6-tetrahydro-2H-pyrano[3,4-c]isoquinolin- | |
| | 1-yl)-N-methyl-1H-pyrrolo[3,2-c]pyridine-2-carboxamide | |
| 309 | 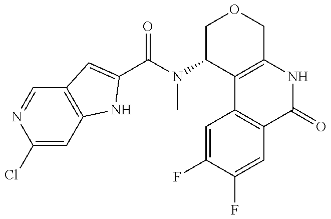 [SMILES: CN(C(=O)c1cc2cnc(Cl)cc2[nH]1)[C@@H]1COCc2[nH]c(=O)c3cc(F)c(F)cc3c21] | 0.05 |
| | (S)-6-chloro-N-(8,9-difluoro-6-oxo-1,4,5,6-tetrahydro-2H-pyrano[3,4- | |
| | c]isoquinolin-1-yl)-N-methyl-1H-pyrrolo[3,2-c]pyridine-2-carboxamide | |
| 310 | 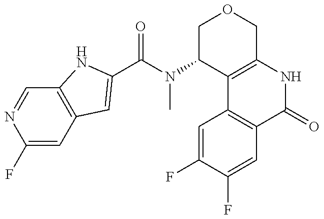 [SMILES: CN(C(=O)c1cc2cc(F)ncc2[nH]1)[C@@H]1COCc2[nH]c(=O)c3cc(F)c(F)cc3c21] | 0.03 |
| | (S)-N-(8,9-difluoro-6-oxo-1,4,5,6-tetrahydro-2H-pyrano[3,4-c]isoquinolin- | |
| | 1-yl)-5-fluoro-N-methyl-1H-pyrrolo[2,3-c]pyridine-2-carboxamide | |
| 311 | 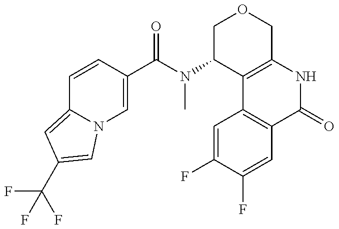 [SMILES: CN(C(=O)c1ccc2cc(C(F)(F)F)cn2c1)[C@@H]1COCc2[nH]c(=O)c3cc(F)c(F)cc3c21] | 0.14 |
| | (S)-N-(8,9-difluoro-6-oxo-1,4,5,6-tetrahydro-2H-pyrano[3,4-c]isoquinolin- | |
| | 1-yl)-N-methyl-2-(trifluoromethyl)indolizine-6-carboxamide | |
| 312 | 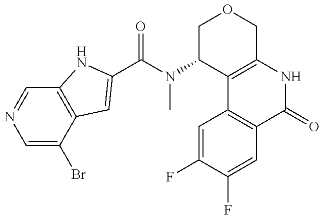 [SMILES: CN(C(=O)c1cc2c(Br)cncc2[nH]1)[C@@H]1COCc2[nH]c(=O)c3cc(F)c(F)cc3c21] | 0.03 |
| | (S)-4-bromo-N-(8,9-difluoro-6-oxo-1,4,5,6-tetrahydro-2H-pyrano[3,4- | |
| | c]isoquinolin-1-yl)-N-methyl-1H-pyrrolo[2,3-c]pyridine-2-carboxamide | |
| 313 | 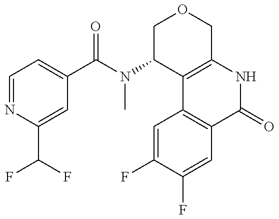 [SMILES: CN(C(=O)c1ccnc(C(F)F)c1)[C@@H]1COCc2[nH]c(=O)c3cc(F)c(F)cc3c21] | 0.17 |
| | (S)-N-(8,9-difluoro-6-oxo-1,4,5,6-tetrahydro-2H-pyrano[3,4-c]isoquinolin- | |
| | 1-yl)-2-(difluoromethyl)-N-methylisonicotinamide | |
| 314 | 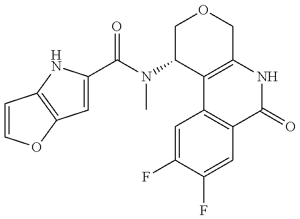 [SMILES: CN(C(=O)c1cc2occc2[nH]1)[C@@H]1COCc2[nH]c(=O)c3cc(F)c(F)cc3c21] | 0.15 |
| | (S)-N-(8,9-difluoro-6-oxo-1,4,5,6-tetrahydro-2H-pyrano[3,4-c]isoquinolin- | |
| | 1-yl)-N-methyl-4H-furo[3,2-b]pyrrole-5-carboxamide | |
| 315 | 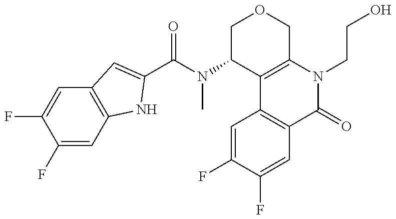 [SMILES: CN(C(=O)c1cc2cc(F)c(F)cc2[nH]1)[C@@H]1COCc2c1c1cc(F)c(F)cc1c(=O)n2CCO] | 0.03 |
| | (S)-N-(8,9-difluoro-5-(2-hydroxyethyl)-6-oxo-1,4,5,6-tetrahydro-2H- | |
| | pyrano[3,4-c]isoquinolin-1-y1)-5,6-difluoro-N-methyl-1H-indole-2- | |
| | carboxamide | |
| 316 | 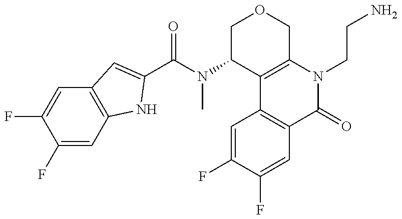 [SMILES: CN(C(=O)c1cc2cc(F)c(F)cc2[nH]1)[C@@H]1COCc2c1c1cc(F)c(F)cc1c(=O)n2CCN] | 0.39 |
| | (S)-N-(5-(2-aminoethyl)-8,9-difluoro-6-oxo-1,4,5,6-tetrahydro-2H- | |
| | pyrano[3,4-c]isoquinolin-1-y1)-5,6-difluoro-N-methyl-1H-indole-2- | |
| | carboxamide | |
| 317 | 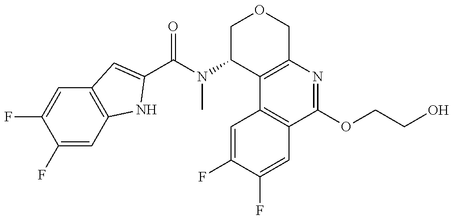 [SMILES: CN(C(=O)c1cc2cc(F)c(F)cc2[nH]1)[C@@H]1COCc2nc(OCCO)c3cc(F)c(F)cc3c21] | 0.01 |
| | (S)-N-(8,9-difluoro-6-(2-hydroxyethoxy)-1,4-dihydro-2H-pyrano[3,4- | |
| | c]isoquinolin-1-yl)-5,6-difluoro-N-methyl-1H-indole-2-carboxamide | |
| 319 | 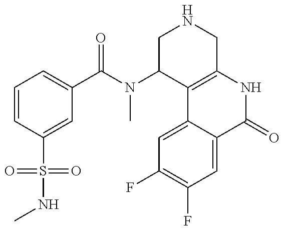 [SMILES: CNS(=O)(=O)c1cccc(C(=O)N(C)C2CNCc3[nH]c(=O)c4cc(F)c(F)cc4c32)c1] | 6.8 |
| | N-(8,9-difluoro-6-oxo-1,2,3,4,5,6-hexahydrobenzo[c][1,7]naphthyridin-1- | |
| | yl)-N-methyl-3-(N-methylsulfamoyl)benzamide | |
| | enantiomer II | |
| 320 | 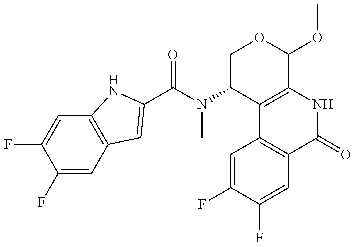 [SMILES: COC1OC[C@@H](N(C)C(=O)c2cc3cc(F)c(F)cc3[nH]2)c2c1[nH]c(=O)c1cc(F)c(F)cc21] | 0.03 |
| | N-((1S)-8,9-difluoro-4-methoxy-6-oxo-1,4,5,6-tetrahydro-2H-pyrano[3,4- | |
| | c]isoquinolin-1-y1)-5,6-difluoro-N-methyl-1H-indole-2-carboxamide | |
| | ~6:1 ratio of epimers at anomeric carbon | |
| 321 | 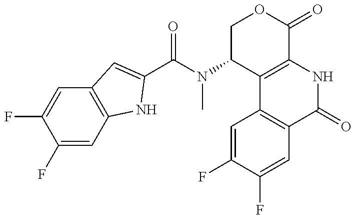 [SMILES: CN(C(=O)c1cc2cc(F)c(F)cc2[nH]1)[C@@H]1COC(=O)c2[nH]c(=O)c3cc(F)c(F)cc3c21] | 0.01 |
| | (S)-N-(8,9-difluoro-4,6-dioxo-1,4,5,6-tetrahydro-2H-pyrano[3,4- | |
| | c]isoquinolin-1-yl)-5,6-difluoro-N-methyl-1H-indole-2-carboxamide | |
| 322 | 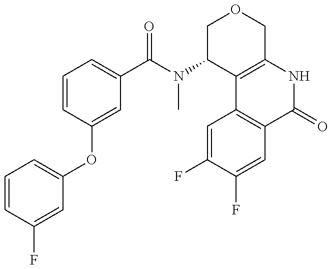 [SMILES: CN(C(=O)c1cccc(Oc2cccc(F)c2)c1)[C@@H]1COCc2[nH]c(=O)c3cc(F)c(F)cc3c21] | 8.1 |
| | (S)-N-(8,9-difluoro-6-oxo-1,4,5,6-tetrahydro-2H-pyrano[3,4-c]isoquinolin- | |
| | 1-y1)-3-(3-fluorophenoxy)-N-methylbenzamide | |
| 324 | 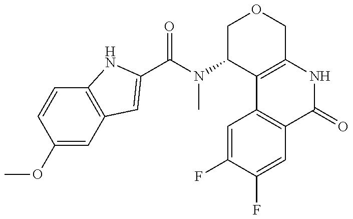 [SMILES: COc1ccc2[nH]c(C(=O)N(C)[C@@H]3COCc4[nH]c(=O)c5cc(F)c(F)cc5c43)cc2c1] | 0.07 |
| | (S)-N-(8,9-difluoro-6-oxo-1,4,5,6-tetrahydro-2H-pyrano[3,4-c]isoquinolin- | |
| | 1-y1)-5-methoxy-N-methyl-1H-indole-2-carboxamide | |
| 325 | 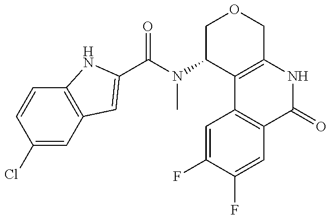 [SMILES: CN(C(=O)c1cc2cc(Cl)ccc2[nH]1)[C@@H]1COCc2[nH]c(=O)c3cc(F)c(F)cc3c21] | 0.02 |
| | (S)-5-chloro-N-(8,9-difluoro-6-oxo-1,4,5,6-tetrahydro-2H-pyrano[3,4- | |
| | c]isoquinolin-1-yl)-N-methyl-1H-indole-2-carboxamide | |
| 326 | 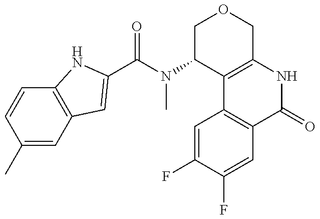 [SMILES: Cc1ccc2[nH]c(C(=O)N(C)[C@@H]3COCc4[nH]c(=O)c5cc(F)c(F)cc5c43)cc2c1] | 2.5 |
| | (S)-N-(8,9-difluoro-6-oxo-1,4,5,6-tetrahydro-2H-pyrano[3,4-c]isoquinolin- | |
| | 1-yl)-N,5-dimethyl-1H-indole-2-carboxamide | |
| 327 | 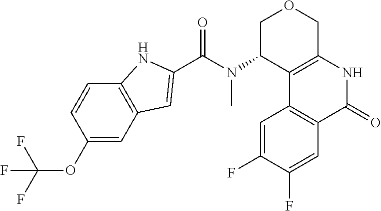 [SMILES: CN(C(=O)c1cc2cc(OC(F)(F)F)ccc2[nH]1)[C@@H]1COCc2[nH]c(=O)c3cc(F)c(F)cc3c21] | 1.2 |
| | (S)-N-(8,9-difluoro-6-oxo-1,4,5,6-tetrahydro-2H-pyrano[3,4-c]isoquinolin- | |
| | 1-yl)-N-methyl-5-(trifluoromethoxy)-1H-indole-2-carboxamide | |
| 328 | 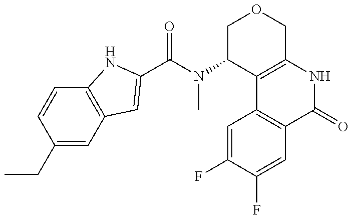 [SMILES: CCc1ccc2[nH]c(C(=O)N(C)[C@@H]3COCc4[nH]c(=O)c5cc(F)c(F)cc5c43)cc2c1] | 3.1 |
| | (S)-N-(8,9-difluoro-6-oxo-1,4,5,6-tetrahydro-2H-pyrano[3,4-c]isoquinolin- | |
| | 1-y1)-5-ethyl-N-methyl-1H-indole-2-carboxamide | |
| 329 | 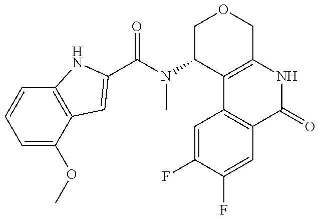 [SMILES: COc1cccc2[nH]c(C(=O)N(C)[C@@H]3COCc4[nH]c(=O)c5cc(F)c(F)cc5c43)cc12] | 0.26 |
| | (S)-N-(8,9-difluoro-6-oxo-1,4,5,6-tetrahydro-2H-pyrano[3,4-c]isoquinolin- | |
| | 1-yl)-4-methoxy-N-methyl-1H-indole-2-carboxamide | |
| 330 | 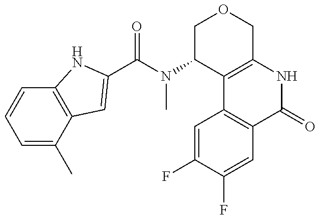 [SMILES: Cc1cccc2[nH]c(C(=O)N(C)[C@@H]3COCc4[nH]c(=O)c5cc(F)c(F)cc5c43)cc12] | 0.01 |
| | (S)-N-(8,9-difluoro-6-oxo-1,4,5,6-tetrahydro-2H-pyrano[3,4-c]isoquinolin- | |
| | 1-yl)-N,4-dimethyl-1H-indole-2-carboxamide | |
| 331 | 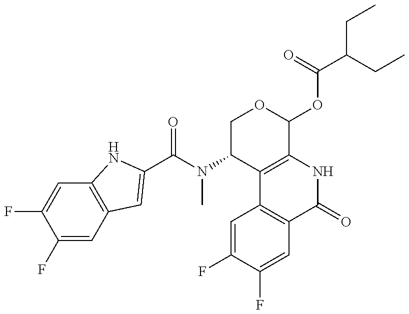 [SMILES: CCC(CC)C(=O)OC1OC[C@@H](N(C)C(=O)c2cc3cc(F)c(F)cc3[nH]2)c2c1[nH]c(=O)c1cc(F)c(F)cc21] | 0.01 |
| | (1S)-1-(5,6-difluoro-N-methyl-1H-indole-2-carboxamido)-8,9-difluoro-6- | |
| | oxo-1,4,5,6-tetrahydro-2H-pyrano[3,4-c]isoquinolin-4-yl 2-ethylbutanoate | |
| | ~6:1 ratio of epimers at anomeric carbon | |
| 332 | 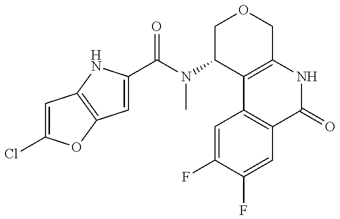 [SMILES: CN(C(=O)c1cc2oc(Cl)cc2[nH]1)[C@@H]1COCc2[nH]c(=O)c3cc(F)c(F)cc3c21] | 0.04 |
| | (S)-2-chloro-N-(8,9-difluoro-6-oxo-1,4,5,6-tetrahydro-2H-pyrano[3,4- | |
| | c]isoquinolin-1-y1)-N-methyl-4H-furo[3,2-b]pyrrole-5-carboxamide | |
| 333 | 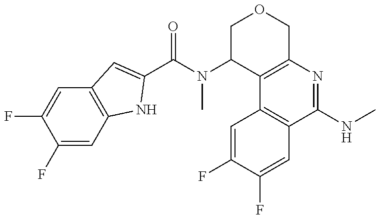 [SMILES: CNc1nc2c(c3cc(F)c(F)cc13)C(N(C)C(=O)c1cc3cc(F)c(F)cc3[nH]1)COC2] | 0.53 |
| | N-(8,9-difluoro-6-(methylamino)-1,4-dihydro-2H-pyrano[3,4-c]isoquinolin- | |
| | 1-yl)-5,6-difluoro-N-methyl-1H-indole-2-carboxamide | |
| | enantiomer I | |
| 334 | 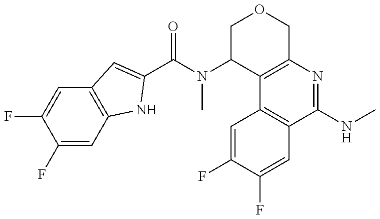 [SMILES: CNc1nc2c(c3cc(F)c(F)cc13)C(N(C)C(=O)c1cc3cc(F)c(F)cc3[nH]1)COC2] | 0.01 |
| | N-(8,9-difluoro-6-(methylamino)-1,4-dihydro-2H-pyrano[3,4-c]isoquinolin- | |
| | 1-yl)-5,6-difluoro-N-methyl-1H-indole-2-carboxamide | |
| | enantiomer II | |
| 335 | 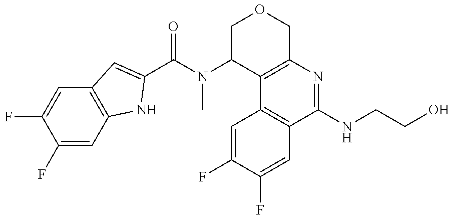 [SMILES: CN(C(=O)c1cc2cc(F)c(F)cc2[nH]1)C1COCc2nc(NCCO)c3cc(F)c(F)cc3c21] | 0.44 |
| | N-(8,9-difluoro-6-((2-hydroxyethyl)amino)-1,4-dihydro-2H-pyrano[3,4- | |
| | c]isoquinolin-1-yl)-5,6-difluoro-N-methyl-1H-indole-2-carboxamide | |
| | enantiomer I | |
| 336 | 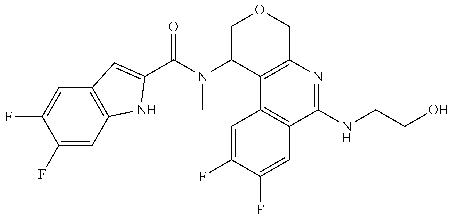 [SMILES: CN(C(=O)c1cc2cc(F)c(F)cc2[nH]1)C1COCc2nc(NCCO)c3cc(F)c(F)cc3c21] | 0.01 |
| | N-(8,9-difluoro-6-((2-hydroxyethyl)amino)-1,4-dihydro-2H-pyrano[3,4- | |
| | c]isoquinolin-1-yl)-5,6-difluoro-N-methyl-1H-indole-2-carboxamide | |
| | enantiomer II | |
| 339 | 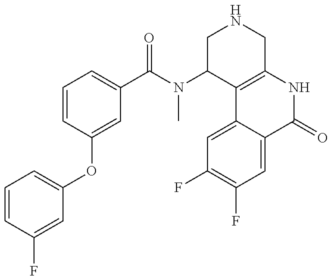 [SMILES: CN(C(=O)c1cccc(Oc2cccc(F)c2)c1)C1CNCc2[nH]c(=O)c3cc(F)c(F)cc3c21] | 25 |
| | N-(8,9-difluoro-6-oxo-1,2,3,4,5,6-hexahydrobenzo[c][1,7]naphthyridin-1- | |
| | yl)-3-(3-fluorophenoxy)-N-methylbenzamide | |
| | enantiomer I | |
| 340 | 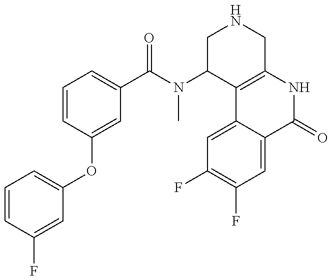 [SMILES: CN(C(=O)c1cccc(Oc2cccc(F)c2)c1)C1CNCc2[nH]c(=O)c3cc(F)c(F)cc3c21] | 25 |
| | N-(8,9-difluoro-6-oxo-1,2,3,4,5,6-hexahydrobenzo[c][1,7]naphthyridin-1- | |
| | y1)-3-(3-fluorophenoxy)-N-methylbenzamide | |
| | enantiomer II | |
| 341 | 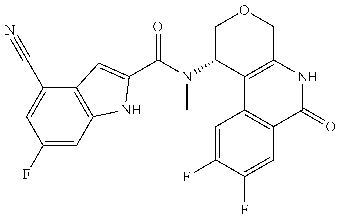 [SMILES: CN(C(=O)c1cc2c(C#N)cc(F)cc2[nH]1)[C@@H]1COCc2[nH]c(=O)c3cc(F)c(F)cc3c21] | 0.03 |
| | (S)-4-cyano-N-(8,9-difluoro-6-oxo-1,4,5,6-tetrahydro-2H-pyrano[3,4- | |
| | c]isoquinolin-1-yl)-6-fluoro-N-methyl-1H-indole-2-carboxamide | |
| 342 | 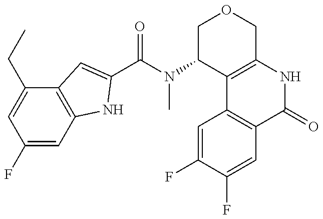 [SMILES: CCc1cc(F)cc2[nH]c(C(=O)N(C)[C@@H]3COCc4[nH]c(=O)c5cc(F)c(F)cc5c43)cc12] | 0.01 |
| | (S)-N-(8,9-difluoro-6-oxo-1,4,5,6-tetrahydro-2H-pyrano[3,4-c]isoquinolin- | |
| | 1-yl)-4-ethyl-6-fluoro-N-methyl-1H-indole-2-carboxamide | |
| 343 | 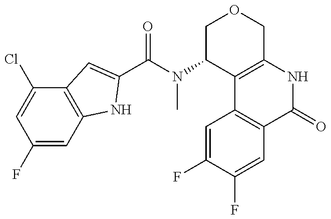 [SMILES: CN(C(=O)c1cc2c(Cl)cc(F)cc2[nH]1)[C@@H]1COCc2[nH]c(=O)c3cc(F)c(F)cc3c21] | 0.01 |
| | (S)-4-chloro-N-(8,9-difluoro-6-oxo-1,4,5,6-tetrahydro-2H-pyrano[3,4- | |
| | c]isoquinolin-1-yl)-6-fluoro-N-methyl-1H-indole-2-carboxamide | |
| 344 | 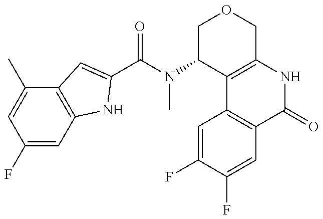 [SMILES: Cc1cc(F)cc2[nH]c(C(=O)N(C)[C@@H]3COCc4[nH]c(=O)c5cc(F)c(F)cc5c43)cc12] | 0.01 |
| | (S)-N-(8,9-difluoro-6-oxo-1,4,5,6-tetrahydro-2H-pyrano[3,4-c]isoquinolin- | |
| | 1-yl)-6-fluoro-N,4-dimethyl-1H-indole-2-carboxamide | |
| 345 | 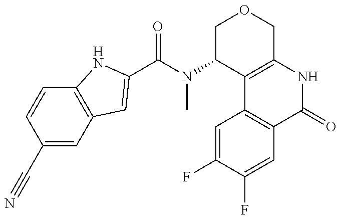 [SMILES: CN(C(=O)c1cc2cc(C#N)ccc2[nH]1)[C@@H]1COCc2[nH]c(=O)c3cc(F)c(F)cc3c21] | 0.03 |
| | (S)-5-cyano-N-(8,9-difluoro-6-oxo-1,4,5,6-tetrahydro-2H-pyrano[3,4- | |
| | c]isoquinolin-1-yl)-N-methyl-1H-indole-2-carboxamide | |
| 346 | 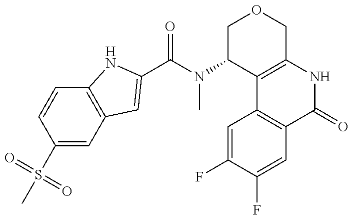 [SMILES: CN(C(=O)c1cc2cc(S(C)(=O)=O)ccc2[nH]1)[C@@H]1COCc2[nH]c(=O)c3cc(F)c(F)cc3c21] | 6.7 |
| | (S)-N-(8,9-difluoro-6-oxo-1,4,5,6-tetrahydro-2H-pyrano[3,4-c]isoquinolin- | |
| | 1-y1)-N-methyl-5-(methylsulfonyl)-1H-indole-2-carboxamide | |
| 347 | 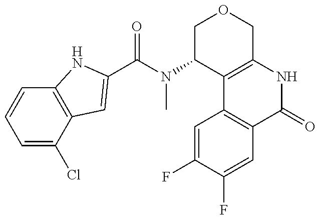 [SMILES: CN(C(=O)c1cc2c(Cl)cccc2[nH]1)[C@@H]1COCc2[nH]c(=O)c3cc(F)c(F)cc3c21] | 0.01 |
| | (S)-4-chloro-N-(8,9-difluoro-6-oxo-1,4,5,6-tetrahydro-2H-pyrano[3,4- | |
| | c]isoquinolin-1-yl)-N-methyl-1H-indole-2-carboxamide | |
| 348 | 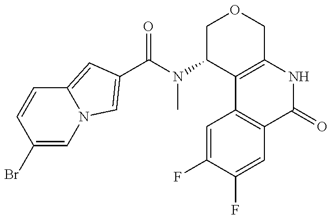 [SMILES: CN(C(=O)c1cc2ccc(Br)cn2c1)[C@@H]1COCc2[nH]c(=O)c3cc(F)c(F)cc3c21] | 0.04 |
| | (S)-6-bromo-N-(8,9-difluoro-6-oxo-1,4,5,6-tetrahydro-2H-pyrano[3,4- | |
| | c]isoquinolin-1-yl)-N-methylindolizine-2-carboxamide | |
| 349 | 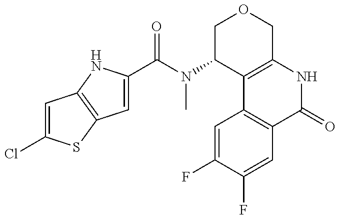 [SMILES: CN(C(=O)c1cc2sc(Cl)cc2[nH]1)[C@@H]1COCc2[nH]c(=O)c3cc(F)c(F)cc3c21] | 0.01 |
| | (S)-2-chloro-N-(8,9-difluoro-6-oxo-1,4,5,6-tetrahydro-2H-pyrano[3,4- | |
| | c]isoquinolin-1-yl)-N-methyl-4H-thieno[3,2-b]pyrrole-5-carboxamide | |
| 350 | 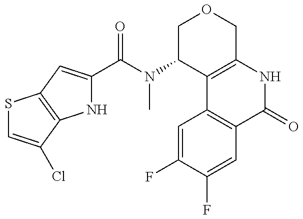 [SMILES: CN(C(=O)c1cc2scc(Cl)c2[nH]1)[C@@H]1COCc2[nH]c(=O)c3cc(F)c(F)cc3c21] | 0.07 |
| | (S)-3-chloro-N-(8,9-difluoro-6-oxo-1,4,5,6-tetrahydro-2H-pyrano[3,4- | |
| | c]isoquinolin-1-yl)-N-methyl-4H-thieno[3,2-b]pyrrole-5-carboxamide | |
| 351 | 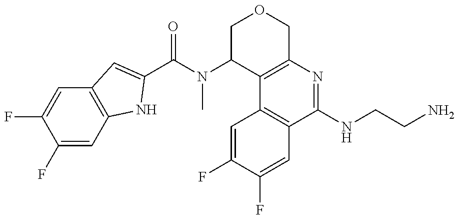 [SMILES: CN(C(=O)c1cc2cc(F)c(F)cc2[nH]1)C1COCc2nc(NCCN)c3cc(F)c(F)cc3c21] | 0.03 |
| | N-(6-((2-aminoethyl)amino)-8,9-difluoro-1,4-dihydro-2H-pyrano[3,4- | |
| | c]isoquinolin-1-y1)-5,6-difluoro-N-methyl-1H-indole-2-carboxamide | |
| | enantiomer I | |
| 352 | 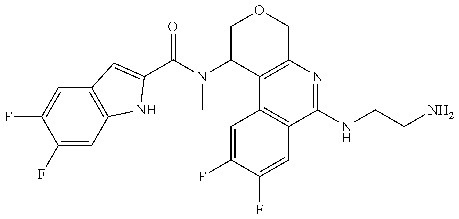 [SMILES: CN(C(=O)c1cc2cc(F)c(F)cc2[nH]1)C1COCc2nc(NCCN)c3cc(F)c(F)cc3c21] | 0.59 |
| | N-(6-((2-aminoethyl)amino)-8,9-difluoro-1,4-dihydro-2H-pyrano[3,4- | |
| | c]isoquinolin-1-yl)-5,6-difluoro-N-methyl-1H-indole-2-carboxamide | |
| | enantiomer II | |
| 353 | 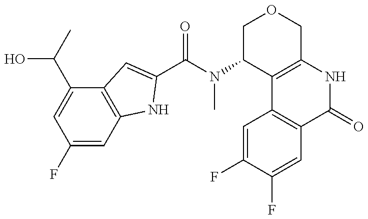 [SMILES: CC(O)c1cc(F)cc2[nH]c(C(=O)N(C)[C@@H]3COCc4[nH]c(=O)c5cc(F)c(F)cc5c43)cc12] | 0.06 |
| | N-((S)-8,9-difluoro-6-oxo-1,4,5,6-tetrahydro-2H-pyrano[3,4-c]isoquinolin- | |
| | 1-yl)-6-fluoro-4-(1-hydroxyethyl)-N-methyl-1H-indole-2-carboxamide | |
| | diastereomer I | |
| 354 | 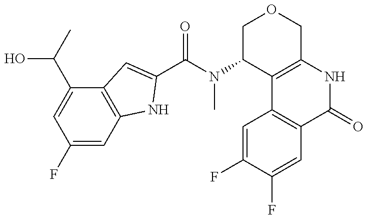 [SMILES: CC(O)c1cc(F)cc2[nH]c(C(=O)N(C)[C@@H]3COCc4[nH]c(=O)c5cc(F)c(F)cc5c43)cc12] | 0.02 |
| | N-((S)-8,9-difluoro-6-oxo-1,4,5,6-tetrahydro-2H-pyrano[3,4-c]isoquinolin- | |
| | 1-y1)-6-fluoro-4-(1-hydroxyethyl)-N-methyl-1H-indole-2-carboxamide | |
| | diastereomer II | |
| 355 | 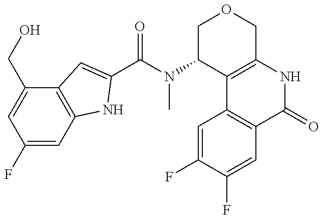 [SMILES: CN(C(=O)c1cc2c(CO)cc(F)cc2[nH]1)[C@@H]1COCc2[nH]c(=O)c3cc(F)c(F)cc3c21] | 0.02 |
| | (S)-N-(8,9-difluoro-6-oxo-1,4,5,6-tetrahydro-2H-pyrano[3,4-c]isoquinolin- | |
| | 1-yl)-6-fluoro-4-(hydroxymethyl)-N-methyl-1H-indole-2-carboxamide | |
| 356 | 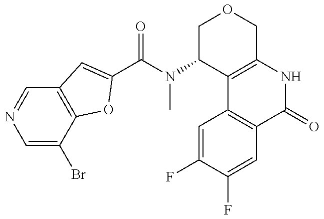 [SMILES: CN(C(=O)c1cc2cncc(Br)c2o1)[C@@H]1COCc2[nH]c(=O)c3cc(F)c(F)cc3c21] | 1.5 |
| | (S)-7-bromo-N-(8,9-difluoro-6-oxo-1,4,5,6-tetrahydro-2H-pyrano[3,4- c] | |
| | isoquinolin-1-yl)-N-methylfuro[3,2-c]pyridine-2-carboxamide | |
| 357 | 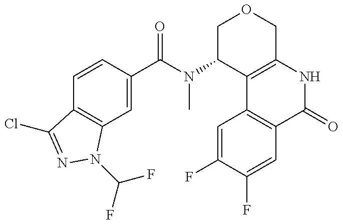 [SMILES: CN(C(=O)c1ccc2c(Cl)nn(C(F)F)c2c1)[C@@H]1COCc2[nH]c(=O)c3cc(F)c(F)cc3c21] | 0.03 |
| | (S)-3-chloro-N-(8,9-difluoro-6-oxo-1,4,5,6-tetrahydro-2H-pyrano[3,4- | |
| | c]isoquinolin-1-yl)-1-(difluoromethyl)-N-methyl-1H-indazole-6- | |
| | carboxamide | |
| 358 | 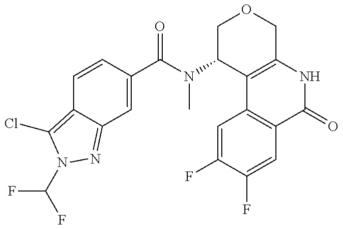 [SMILES: CN(C(=O)c1ccc2c(Cl)n(C(F)F)nc2c1)[C@@H]1COCc2[nH]c(=O)c3cc(F)c(F)cc3c21] | 0.68 |
| | (S)-3-chloro-N-(8,9-difluoro-6-oxo-1,4,5,6-tetrahydro-2H-pyrano[3,4- | |
| | c]isoquinolin-1-yl)-2-(difluoromethyl)-N-methyl-2H-indazole-6- | |
| | carboxamide | |
| 359 | 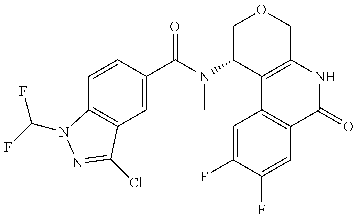 [SMILES: CN(C(=O)c1ccc2c(c1)c(Cl)nn2C(F)F)[C@@H]1COCc2[nH]c(=O)c3cc(F)c(F)cc3c21] | 0.01 |
| | (S)-3-chloro-N-(8,9-difluoro-6-oxo-1,4,5,6-tetrahydro-2H-pyrano[3,4- | |
| | c]isoquinolin-1-yl)-1-(difluoromethyl)-N-methyl-1H-indazole-5- | |
| | carboxamide | |
| 360 | 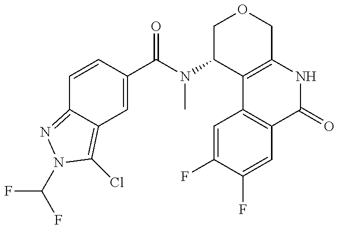 [SMILES: CN(C(=O)c1ccc2nn(C(F)F)c(Cl)c2c1)[C@@H]1COCc2[nH]c(=O)c3cc(F)c(F)cc3c21] | 0.12 |
| | (S)-3-chloro-N-(8,9-difluoro-6-oxo-1,4,5,6-tetrahydro-2H-pyrano[3,4- | |
| | c]isoquinolin-1-yl)-2-(difluoromethyl)-N-methyl-2H-indazole-5- | |
| | carboxamide | |
| 361 | 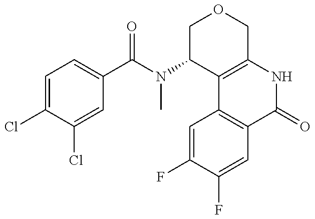 [SMILES: CN(C(=O)c1ccc(Cl)c(Cl)c1)[C@@H]1COCc2[nH]c(=O)c3cc(F)c(F)cc3c21] | 0.01 |
| | (S)-3,4-dichloro-N-(8,9-difluoro-6-oxo-1,4,5,6-tetrahydro-2H-pyrano[3,4- | |
| | c]isoquinolin-1-y1)-N-methylbenzamide | |
| 362 | 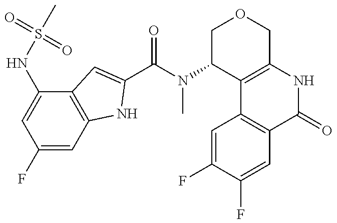 [SMILES: CN(C(=O)c1cc2c(NS(C)(=O)=O)cc(F)cc2[nH]1)[C@@H]1COCc2[nH]c(=O)c3cc(F)c(F)cc3c21] | 14 |
| | (S)-N-(8,9-difluoro-6-oxo-1,4,5,6-tetrahydro-2H-pyrano[3,4-c]isoquinolin- | |
| | 1-yl)-6-fluoro-N-methyl-4-(methylsulfonamido)-1H-indole-2-carboxamide | |
| 363 | 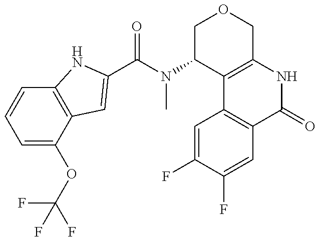 [SMILES: CN(C(=O)c1cc2c(OC(F)(F)F)cccc2[nH]1)[C@@H]1COCc2[nH]c(=O)c3cc(F)c(F)cc3c21] | 0.02 |
| | (S)-N-(8,9-difluoro-6-oxo-1,4,5,6-tetrahydro-2H-pyrano[3,4-c]isoquinolin- | |
| | 1-yl)-N-methyl-4-(trifluoromethoxy)-1H-indole-2-carboxamide | |
| 364 | 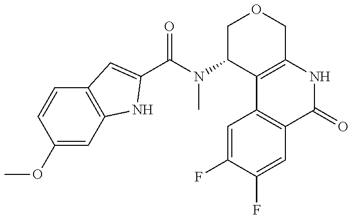 [SMILES: COc1ccc2cc(C(=O)N(C)[C@@H]3COCc4[nH]c(=O)c5cc(F)c(F)cc5c43)[nH]c2c1] | 0.03 |
| | (S)-N-(8,9-difluoro-6-oxo-1,4,5,6-tetrahydro-2H-pyrano[3,4-c]isoquinolin- | |
| | 1-yl)-6-methoxy-N-methyl-1H-indole-2-carboxamide | |
| 365 | 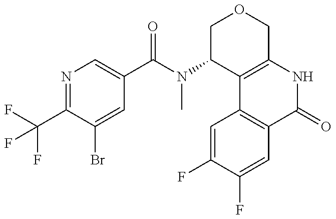 [SMILES: CN(C(=O)c1cnc(C(F)(F)F)c(Br)c1)[C@@H]1COCc2[nH]c(=O)c3cc(F)c(F)cc3c21] | 0.06 |
| | (S)-5-bromo-N-(8,9-difluoro-6-oxo-1,4,5,6-tetrahydro-2H-pyrano[3,4- | |
| | c]isoquinolin-1-yl)-N-methyl-6-(trifluoromethyl)nicotinamide | |
| 366 | 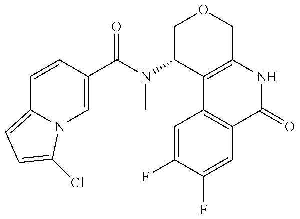 [SMILES: CN(C(=O)c1ccc2ccc(Cl)n2c1)[C@@H]1COCc2[nH]c(=O)c3cc(F)c(F)cc3c21] | 0.10 |
| | (S)-3-chloro-N-(8,9-difluoro-6-oxo-1,4,5,6-tetrahydro-2H-pyrano[3,4- c] | |
| | isoquinolin-1-yl)-N-methylindolizine-6-carboxamide | |
| 367 | 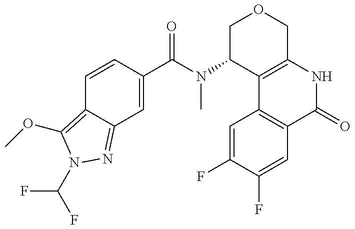 [SMILES: COc1c2ccc(C(=O)N(C)[C@@H]3COCc4[nH]c(=O)c5cc(F)c(F)cc5c43)cc2nn1C(F)F] | 4.6 |
| | (S)-N-(8,9-difluoro-6-oxo-1,4,5,6-tetrahydro-2H-pyrano[3,4-c]isoquinolin- | |
| | 1-yl)-2-(difluoromethyl)-3-methoxy-N-methyl-2H-indazole-6-carboxamide | |
| 368 | 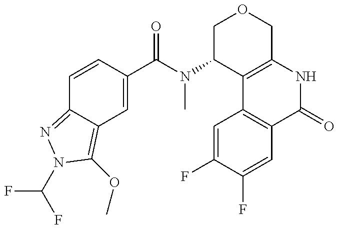 [SMILES: COc1c2cc(C(=O)N(C)[C@@H]3COCc4[nH]c(=O)c5cc(F)c(F)cc5c43)ccc2nn1C(F)F] | 3.4 |
| | (S)-N-(8,9-difluoro-6-oxo-1,4,5,6-tetrahydro-2H-pyrano[3,4-c]isoquinolin- | |
| | 1-yl)-2-(difluoromethyl)-3-methoxy-N-methyl-2H-indazole-5-carboxamide | |
| 369 | 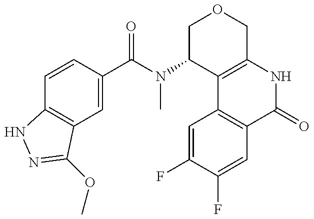 [SMILES: COc1n[nH]c2ccc(C(=O)N(C)[C@@H]3COCc4[nH]c(=O)c5cc(F)c(F)cc5c43)cc12] | 6.1 |
| | (S)-N-(8,9-difluoro-6-oxo-1,4,5,6-tetrahydro-2H-pyrano[3,4-c]isoquinolin- | |
| | 1-yl)-3-methoxy-N-methyl-1H-indazole-5-carboxamide | |
| 370 | 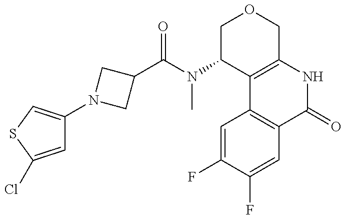 [SMILES: CN(C(=O)C1CN(c2csc(Cl)c2)C1)[C@@H]1COCc2[nH]c(=O)c3cc(F)c(F)cc3c21] | 0.49 |
| | (S)-1-(5-chlorothiophen-3-y1)-N-(8,9-difluoro-6-oxo-1,4,5,6-tetrahydro-2H- | |
| | pyrano[3,4-c]isoquinolin-1-yl)-N-methylazetidine-3-carboxamide | |
| 371 | 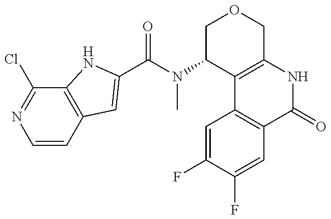 [SMILES: CN(C(=O)c1cc2ccnc(Cl)c2[nH]1)[C@@H]1COCc2[nH]c(=O)c3cc(F)c(F)cc3c21] | 0.14 |
| | (S)-7-chloro-N-(8,9-difluoro-6-oxo-1,4,5,6-tetrahydro-2H-pyrano[3,4- | |
| | c]isoquinolin-1-yl)-N-methyl-1H-pyrrolo[2,3-c]pyridine-2-carboxamide | |
| 372 | 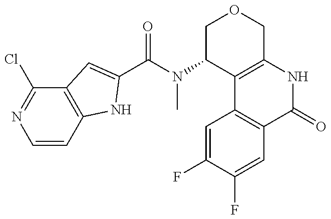 [SMILES: CN(C(=O)c1cc2c(Cl)nccc2[nH]1)[C@@H]1COCc2[nH]c(=O)c3cc(F)c(F)cc3c21] | 0.02 |
| | (S)-4-chloro-N-(8,9-difluoro-6-oxo-1,4,5,6-tetrahydro-2H-pyrano[3,4- | |
| | c]isoquinolin-1-yl)-N-methyl-1H-pyrrolo[3,2-c]pyridine-2-carboxamide | |
| 373 | 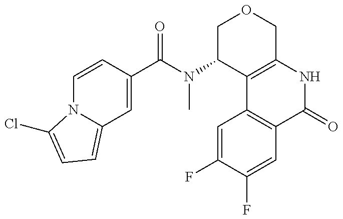 [SMILES: CN(C(=O)c1ccn2c(Cl)ccc2c1)[C@@H]1COCc2[nH]c(=O)c3cc(F)c(F)cc3c21] | 0.01 |
| | (S)-3-chloro-N-(8,9-difluoro-6-oxo-1,4,5,6-tetrahydro-2H-pyrano[3,4- | |
| | c]isoquinolin-1-yl)-N-methylindolizine-7-carboxamide | |
| 374 | 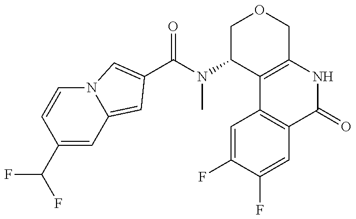 [SMILES: CN(C(=O)c1cc2cc(C(F)F)ccn2c1)[C@@H]1COCc2[nH]c(=O)c3cc(F)c(F)cc3c21] | 24 |
| | (S)-N-(8,9-difluoro-6-oxo-1,4,5,6-tetrahydro-2H-pyrano[3,4-c]isoquinolin- | |
| | 1-y1)-7-(difluoromethyl)-N-methylindolizine-2-carboxamide | |
| 375 | 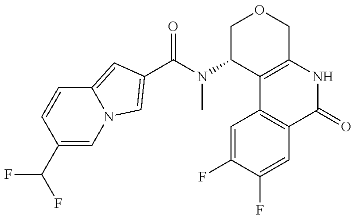 [SMILES: CN(C(=O)c1cc2ccc(C(F)F)cn2c1)[C@@H]1COCc2[nH]c(=O)c3cc(F)c(F)cc3c21] | 0.33 |
| | (S)-N-(8,9-difluoro-6-oxo-1,4,5,6-tetrahydro-2H-pyrano[3,4-c]isoquinolin- | |
| | 1-yl)-6-(difluoromethyl)-N-methylindolizine-2-carboxamide | |
| 376 | 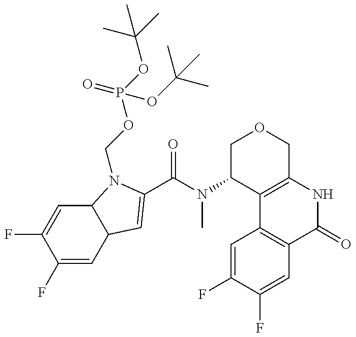 [SMILES: CN(C(=O)C1=CC2C=C(F)C(F)=CC2N1COP(=O)(OC(C)(C)C)OC(C)(C)C)[C@@H]1COCc2[nH]c(=O)c3cc(F)c(F)cc3c21] | 0.01 |
| | (S)-di-tert-butyl ((2-((8,9-difluoro-6-oxo-1,4,5,6-tetrahydro-2H-pyrano[3,4- | |
| | c]isoquinolin-1-yl)(methyl)carbamoyl)-5,6-difluoro-1H-indol-1-yl)methyl) | |
| | phosphate | |
| 377 | 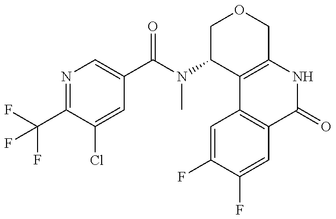 [SMILES: CN(C(=O)c1cnc(C(F)(F)F)c(Cl)c1)[C@@H]1COCc2[nH]c(=O)c3cc(F)c(F)cc3c21] | 0.04 |
| | (S)-5-chloro-N-(8,9-difluoro-6-oxo-1,4,5,6-tetrahydro-2H-pyrano[3,4- | |
| | c]isoquinolin-1-yl)-N-methyl-6-(trifluoromethyl)nicotinamide | |
| 378 | 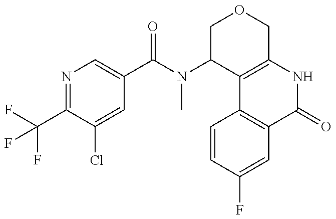 [SMILES: CN(C(=O)c1cnc(C(F)(F)F)c(Cl)c1)C1COCc2[nH]c(=O)c3cc(F)ccc3c21] | 18.3 |
| | 5-chloro-N-(8-fluoro-6-oxo-1,4,5,6-tetrahydro-2H-pyrano[3,4-c]isoquinolin- | |
| | 1-yl)-N-methyl-6-(trifluoromethyl)nicotinamide | |
| | enantiomer I | |
| 379 | 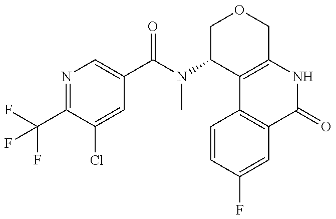 [SMILES: CN(C(=O)c1cnc(C(F)(F)F)c(Cl)c1)[C@@H]1COCc2[nH]c(=O)c3cc(F)ccc3c21] | 0.17 |
| | 5-chloro-N-(8-fluoro-6-oxo-1,4,5,6-tetrahydro-2H-pyrano[3,4-c]isoquinolin- | |
| | 1-yl)-N-methyl-6-(trifluoromethyl)nicotinamide | |
| | enantiomer II | |
| 380 | 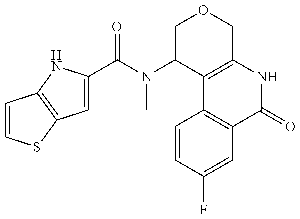 [SMILES: CN(C(=O)c1cc2sccc2[nH]1)C1COCc2[nH]c(=O)c3cc(F)ccc3c21] | 0.88 |
| | N-(8-fluoro-6-oxo-1,4,5,6-tetrahydro-2H-pyrano[3,4-c]isoquinolin-1-yl)-N- | |
| | methyl-4H-thieno[3,2-b]pyrrole-5-carboxamide | |
| | enantiomer I | |
| 381 | 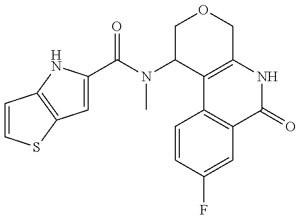 [SMILES: CN(C(=O)c1cc2sccc2[nH]1)C1COCc2[nH]c(=O)c3cc(F)ccc3c21] | 0.04 |
| | N-(8-fluoro-6-oxo-1,4,5,6-tetrahydro-2H-pyrano[3,4-c]isoquinolin-1-yl)-N- | |
| | methyl-4H-thieno[3,2-b]pyrrole-5-carboxamide | |
| | enantiomer II | |
| 382 | 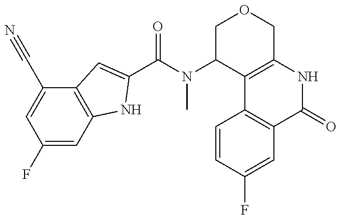 [SMILES: CN(C(=O)c1cc2c(C#N)cc(F)cc2[nH]1)C1COCc2[nH]c(=O)c3cc(F)ccc3c21] | 0.98 |
| | 4-cyano-6-fluoro-N-(8-fluoro-6-oxo-1,4,5,6-tetrahydro-2H-pyrano[3,4- | |
| | c]isoquinolin-1-yl)-N-methyl-1H-indole-2-carboxamide | |
| | enantiomer I | |
| 383 | 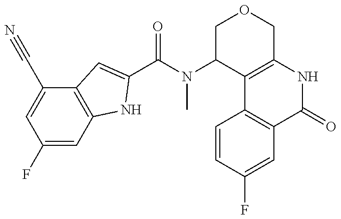 [SMILES: CN(C(=O)c1cc2c(C#N)cc(F)cc2[nH]1)C1COCc2[nH]c(=O)c3cc(F)ccc3c21] | 0.04 |
| | 4-cyano-6-fluoro-N-(8-fluoro-6-oxo-1,4,5,6-tetrahydro-2H-pyrano[3,4- | |
| | c]isoquinolin-1-yl)-N-methyl-1H-indole-2-carboxamide | |
| | enantiomer II | |
| 384 | 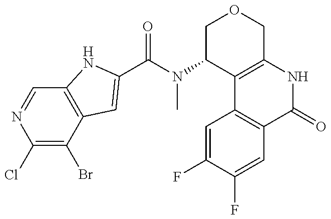 [SMILES: CN(C(=O)c1cc2c(Br)c(Cl)ncc2[nH]1)[C@@H]1COCc2[nH]c(=O)c3cc(F)c(F)cc3c21] | 0.02 |
| | (S)-4-bromo-5-chloro-N-(8,9-difluoro-6-oxo-1,4,5,6-tetrahydro-2H- | |
| | pyrano[3,4-c]isoquinolin-1-yl)-N-methyl-1H-pyrrolo[2,3-c]pyridine-2- | |
| | carboxamide | |
| 386 | 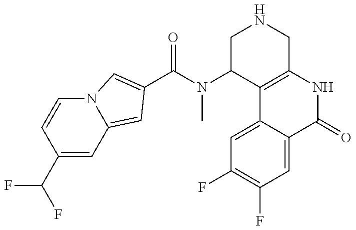 [SMILES: CN(C(=O)c1cc2cc(C(F)F)ccn2c1)C1CNCc2[nH]c(=O)c3cc(F)c(F)cc3c21] | 1.9 |
| | N-(8,9-difluoro-6-oxo-1,2,3,4,5,6-hexahydrobenzo[c][1,7]naphthyridin-1- | |
| | yl)-7-(difluoromethyl)-N-methylindolizine-2-carboxamide | |
| | enantiomer II | |
| 387 | 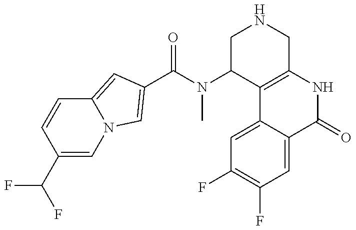 [SMILES: CN(C(=O)c1cc2ccc(C(F)F)cn2c1)C1CNCc2[nH]c(=O)c3cc(F)c(F)cc3c21] | 11 |
| | N-(8,9-difluoro-6-oxo-1,2,3,4,5,6-hexahydrobenzo[c][1,7]naphthyridin-1- | |
| | yl)-6-(difluoromethyl)-N-methylindolizine-2-carboxamide | |
| | enantiomer I | |
| 388 | 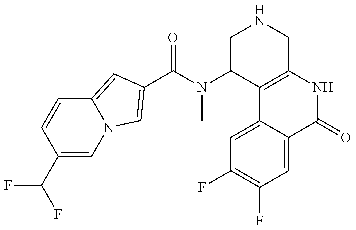 [SMILES: CN(C(=O)c1cc2ccc(C(F)F)cn2c1)C1CNCc2[nH]c(=O)c3cc(F)c(F)cc3c21] | 0.11 |
| | N-(8,9-difluoro-6-oxo-1,2,3,4,5,6-hexahydrobenzo[c][1,7]naphthyridin-1- | |
| | yl)-6-(difluoromethyl)-N-methylindolizine-2-carboxamide | |
| | enantiomer II | |
| 389 | 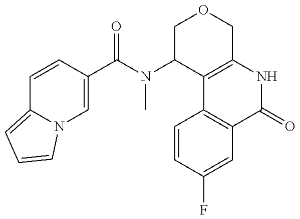 [SMILES: CN(C(=O)c1ccc2cccn2c1)C1COCc2[nH]c(=O)c3cc(F)ccc3c21] | 17 |
| | N-(8-fluoro-6-oxo-1,4,5,6-tetrahydro-2H-pyrano[3,4-c]isoquinolin-1-yl)-N- | |
| | methylindolizine-6-carboxamide | |
| | enantiomer I | |
| 390 | 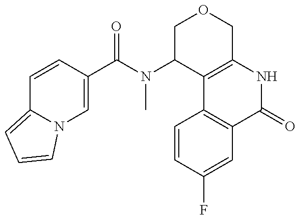 [SMILES: CN(C(=O)c1ccc2cccn2c1)C1COCc2[nH]c(=O)c3cc(F)ccc3c21] | 0.07 |
| | N-(8-fluoro-6-oxo-1,4,5,6-tetrahydro-2H-pyrano[3,4-c]isoquinolin-1-yl)-N- | |
| | methylindolizine-6-carboxamide | |
| | enantiomer II | |
| 391 | 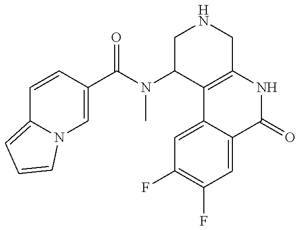 [SMILES: CN(C(=O)c1ccc2cccn2c1)C1CNCc2[nH]c(=O)c3cc(F)c(F)cc3c21] | 22 |
| | N-(8,9-difluoro-6-oxo-1,2,3,4,5,6-hexahydrobenzo[c][1,7]naphthyridin-1- | |
| | yl)-N-methylindolizine-6-carboxamide | |
| | enantiomer I | |
| 392 | 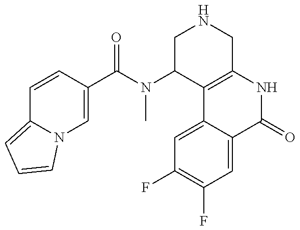 [SMILES: CN(C(=O)c1ccc2cccn2c1)C1CNCc2[nH]c(=O)c3cc(F)c(F)cc3c21] | 0.09 |
| | N-(8,9-difluoro-6-oxo-1,2,3,4,5,6-hexahydrobenzo[c][1,7]naphthyridin-1- | |
| | yl)-N-methylindolizine-6-carboxamide | |
| | enantiomer II | |
| 393 | 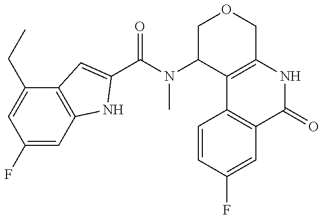 [SMILES: CCc1cc(F)cc2[nH]c(C(=O)N(C)C3COCc4[nH]c(=O)c5cc(F)ccc5c43)cc12] | 1.20 |
| | 4-ethyl-6-fluoro-N-(8-fluoro-6-oxo-1,4,5,6-tetrahydro-2H-pyrano[3,4- | |
| | c]isoquinolin-1-yl)-N-methyl-1H-indole-2-carboxamide | |
| | enantiomer I | |
| 394 | 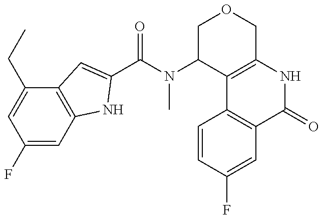 [SMILES: CCc1cc(F)cc2[nH]c(C(=O)N(C)C3COCc4[nH]c(=O)c5cc(F)ccc5c43)cc12] | 0.46 |
| | 4-ethyl-6-fluoro-N-(8-fluoro-6-oxo-1,4,5,6-tetrahydro-2H-pyrano[3,4- | |
| | c]isoquinolin-1-y1)-N-methyl-1H-indole-2-carboxamide | |
| | enantiomer II | |
| 397 | 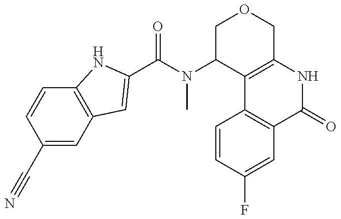 [SMILES: CN(C(=O)c1cc2cc(C#N)ccc2[nH]1)C1COCc2[nH]c(=O)c3cc(F)ccc3c21] | 17 |
| | 5-cyano-N-(8-fluoro-6-oxo-1,4,5,6-tetrahydro-2H-pyrano[3,4-c]isoquinolin- | |
| | 1-yl)-N-methyl-1H-indole-2-carboxamide | |
| | enantiomer I | |
| 398 | 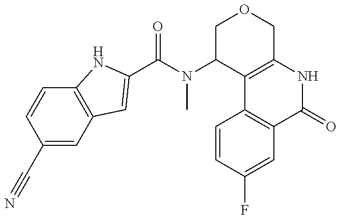 [SMILES: CN(C(=O)c1cc2cc(C#N)ccc2[nH]1)C1COCc2[nH]c(=O)c3cc(F)ccc3c21] | 0.27 |
| | 5-cyano-N-(8-fluoro-6-oxo-1,4,5,6-tetrahydro-2H-pyrano[3,4-c]isoquinolin- | |
| | 1-yl)-N-methyl-1H-indole-2-carboxamide | |
| | enantiomer II | |
| 399 | 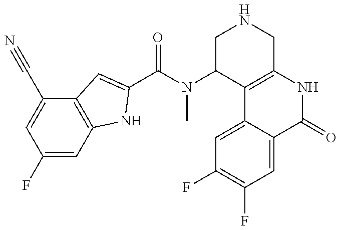 [SMILES: CN(C(=O)c1cc2c(C#N)cc(F)cc2[nH]1)C1CNCc2[nH]c(=O)c3cc(F)c(F)cc3c21] | 3.8 |
| | 4-cyano-N-(8,9-difluoro-6-oxo-1,2,3,4,5,6- | |
| | hexahydrobenzo[c][1,7]naphthyridin-1-yl)-6-fluoro-N-methyl-1H-indole-2- | |
| | carboxamide | |
| | enantiomer I | |
| 400 | 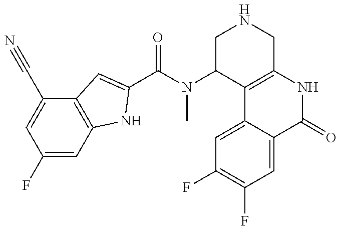 [SMILES: CN(C(=O)c1cc2c(C#N)cc(F)cc2[nH]1)C1CNCc2[nH]c(=O)c3cc(F)c(F)cc3c21] | 0.09 |
| | 4-cyano-N-(8,9-difluoro-6-oxo-1,2,3,4,5,6- | |
| | hexahydrobenzo[c][1,7]naphthyridin-1-yl)-6-fluoro-N-methyl-1H-indole-2- | |
| | carboxamide | |
| | enantiomer II | |
| 401 | 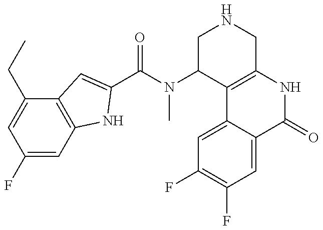 [SMILES: CCc1cc(F)cc2[nH]c(C(=O)N(C)C3CNCc4[nH]c(=O)c5cc(F)c(F)cc5c43)cc12] | 2.5 |
| | N-(8,9-difluoro-6-oxo-1,2,3,4,5,6-hexahydrobenzo[c][1,7]naphthyridin-1- | |
| | yl)-4-ethyl-6-fluoro-N-methyl-1H-indole-2-carboxamide | |
| | enantiomer I | |
| 402 | 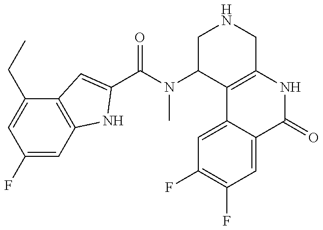 [SMILES: CCc1cc(F)cc2[nH]c(C(=O)N(C)C3CNCc4[nH]c(=O)c5cc(F)c(F)cc5c43)cc12] | 0.02 |
| | N-(8,9-difluoro-6-oxo-1,2,3,4,5,6-hexahydrobenzo[c][1,7]naphthyridin-1- | |
| | yl)-4-ethyl-6-fluoro-N-methyl-1H-indole-2-carboxamide | |
| | enantiomer II | |
| 403 | 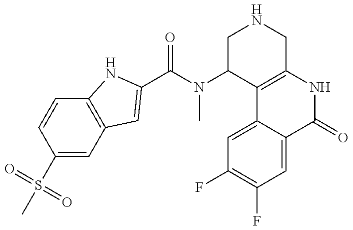 [SMILES: CN(C(=O)c1cc2cc(S(C)(=O)=O)ccc2[nH]1)C1CNCc2[nH]c(=O)c3cc(F)c(F)cc3c21] | 25 |
| | N-(8,9-difluoro-6-oxo-1,2,3,4,5,6-hexahydrobenzo[c][1,7]naphthyridin-1- | |
| | yl)-N-methyl-5-(methylsulfonyl)-1H-indole-2-carboxamide | |
| | enantiomer I | |
| 406 | 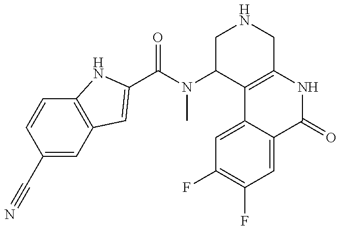 [SMILES: CN(C(=O)c1cc2cc(C#N)ccc2[nH]1)C1CNCc2[nH]c(=O)c3cc(F)c(F)cc3c21] | 0.19 |
| | 5-cyano-N-(8,9-difluoro-6-oxo-1,2,3,4,5,6- | |
| | hexahydrobenzo[c][1,7]naphthyridin-1-yl)-N-methyl-1H-indole-2- | |
| | carboxamide | |
| | enantiomer II | |
| 407 | 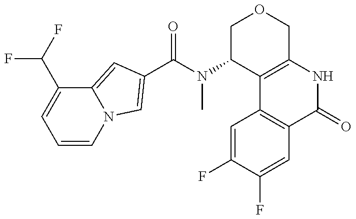 [SMILES: CN(C(=O)c1cc2c(C(F)F)cccn2c1)[C@@H]1COCc2[nH]c(=O)c3cc(F)c(F)cc3c21] | 0.01 |
| | (S)-N-(8,9-difluoro-6-oxo-1,4,5,6-tetrahydro-2H-pyrano[3,4-c]isoquinolin- | |
| | 1-y1)-8-(difluoromethyl)-N-methylindolizine-2-carboxamide | |
| 408 | 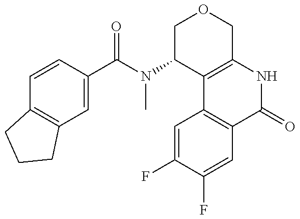 [SMILES: CN(C(=O)c1ccc2c(c1)CCC2)[C@@H]1COCc2[nH]c(=O)c3cc(F)c(F)cc3c21] | 0.04 |
| | (S)-N-(8,9-difluoro-6-oxo-1,4,5,6-tetrahydro-2H-pyrano[3,4-c]isoquinolin- | |
| | 1-yl)-N-methyl-2,3-dihydro-1H-indene-5-carboxamide | |
| 409 | 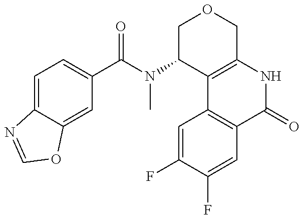 [SMILES: CN(C(=O)c1ccc2ncoc2c1)[C@@H]1COCc2[nH]c(=O)c3cc(F)c(F)cc3c21] | 2.3 |
| | F | |
| | (S)-N-(8,9-difluoro-6-oxo-1,4,5,6-tetrahydro-2H-pyrano[3,4-c]isoquinolin- | |
| | 1-y1)-N-methylbenzo[d]oxazole-6-carboxamide | |
| 410 | 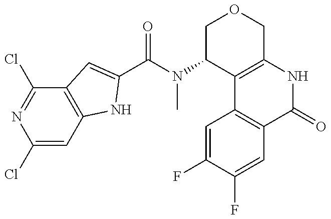 [SMILES: CN(C(=O)c1cc2c(Cl)nc(Cl)cc2[nH]1)[C@@H]1COCc2[nH]c(=O)c3cc(F)c(F)cc3c21] | 0.04 |
| | (S)-4,6-dichloro-N-(8,9-difluoro-6-oxo-1,4,5,6-tetrahydro-2H-pyrano[3,4- | |
| | c]isoquinolin-1-yl)-N-methyl-1H-pyrrolo[3,2-c]pyridine-2-carboxamide | |
| 411 | 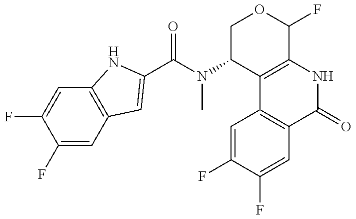 [SMILES: CN(C(=O)c1cc2cc(F)c(F)cc2[nH]1)[C@@H]1COC(F)c2[nH]c(=O)c3cc(F)c(F)cc3c21] | 0.01 |
| | 5,6-difluoro-N-methyl-N-((1S)-4,8,9-trifluoro-6-oxo-1,4,5,6-tetrahydro-2H- | |
| | pyrano[3,4-c]isoquinolin-1-y1)-1H-indole-2-carboxamide | |
| | ~6:1 ratio of epimers at fluorine substituted carbon | |
| 412 | 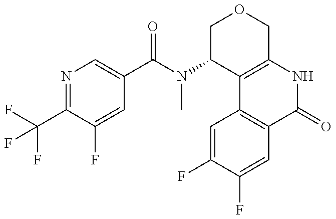 [SMILES: CN(C(=O)c1cnc(C(F)(F)F)c(F)c1)[C@@H]1COCc2[nH]c(=O)c3cc(F)c(F)cc3c21] | 0.03 |
| | (S)-N-(8,9-difluoro-6-oxo-1,4,5,6-tetrahydro-2H-pyrano[3,4-c]isoquinolin- | |
| | 1-y1)-5-fluoro-N-methyl-6-(trifluoromethyl)nicotinamide | |
| 413 | 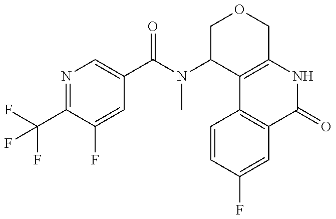 [SMILES: CN(C(=O)c1cnc(C(F)(F)F)c(F)c1)C1COCc2[nH]c(=O)c3cc(F)ccc3c21] | 25 |
| | 5-fluoro-N-(8-fluoro-6-oxo-1,4,5,6-tetrahydro-2H-pyrano[3,4-c]isoquinolin- | |
| | 1-y1)-N-methyl-6-(trifluoromethyl)nicotinamide | |
| | enantiomer I | |
| 414 | 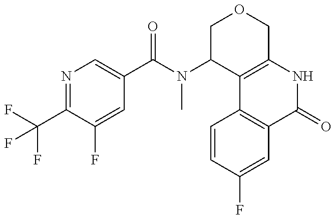 [SMILES: CN(C(=O)c1cnc(C(F)(F)F)c(F)c1)C1COCc2[nH]c(=O)c3cc(F)ccc3c21] | 0.17 |
| | 5-fluoro-N-(8-fluoro-6-oxo-1,4,5,6-tetrahydro-2H-pyrano[3,4-c]isoquinolin- | |
| | 1-yl)-N-methyl-6-(trifluoromethyl)nicotinamide | |
| | enantiomer II | |
| 415 | 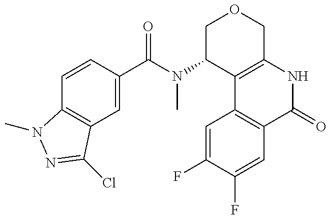 [SMILES: CN(C(=O)c1ccc2c(c1)c(Cl)nn2C)[C@@H]1COCc2[nH]c(=O)c3cc(F)c(F)cc3c21] | 0.06 |
| | (S)-3-chloro-N-(8,9-difluoro-6-oxo-1,4,5,6-tetrahydro-2H-pyrano[3,4- | |
| | c]isoquinolin-1-yl)-N,1-dimethyl-1H-indazole-5-carboxamide | |
| 416 | 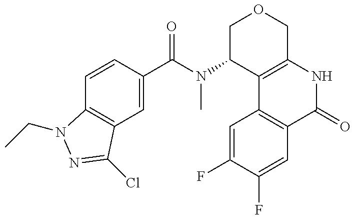 [SMILES: CCn1nc(Cl)c2cc(C(=O)N(C)[C@@H]3COCc4[nH]c(=O)c5cc(F)c(F)cc5c43)ccc21] | 0.08 |
| | (S)-3-chloro-N-(8,9-difluoro-6-oxo-1,4,5,6-tetrahydro-2H-pyrano[3,4- | |
| | c]isoquinolin-1-yl)-1-ethyl-N-methyl-1H-indazole-5-carboxamide | |
| 417 | 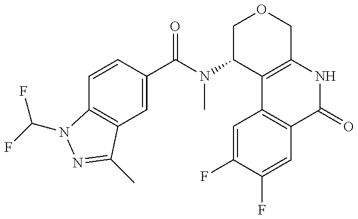 [SMILES: Cc1nn(C(F)F)c2ccc(C(=O)N(C)[C@@H]3COCc4[nH]c(=O)c5cc(F)c(F)cc5c43)cc12] | 0.02 |
| | (S)-N-(8,9-difluoro-6-oxo-1,4,5,6-tetrahydro-2H-pyrano[3,4-c]isoquinolin- | |
| | 1-yl)-1-(difluoromethyl)-N,3-dimethyl-1H-indazole-5-carboxamide | |
| 419 | 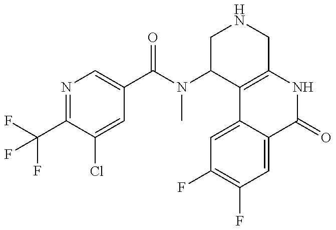 [SMILES: CN(C(=O)c1cnc(C(F)(F)F)c(Cl)c1)C1CNCc2[nH]c(=O)c3cc(F)c(F)cc3c21] | 0.14 |
| | 5-chloro-N-(8,9-difluoro-6-oxo-1,2,3,4,5,6- | |
| | hexahydrobenzo[c][1,7]naphthyridin-1-yl)-N-methyl-6- | |
| | (trifluoromethyl)nicotinamide | |
| | enantiomer II | |
| 420 | 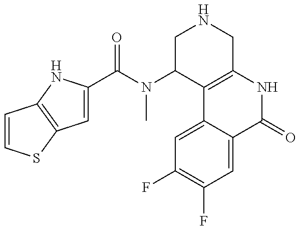 [SMILES: CN(C(=O)c1cc2sccc2[nH]1)C1CNCc2[nH]c(=O)c3cc(F)c(F)cc3c21] | 0.66 |
| | N-(8,9-difluoro-6-oxo-1,2,3,4,5,6-hexahydrobenzo[c][1,7]naphthyridin-1- | |
| | yl)-N-methyl-4H-thieno[3,2-b]pyrrole-5-carboxamide | |
| | enantiomer I | |
| 421 | 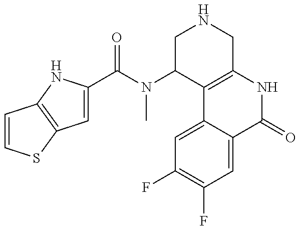 [SMILES: CN(C(=O)c1cc2sccc2[nH]1)C1CNCc2[nH]c(=O)c3cc(F)c(F)cc3c21] | 0.04 |
| | N-(8,9-difluoro-6-oxo-1,2,3,4,5,6-hexahydrobenzo[c][1,7]naphthyridin-1- | |
| | y1)-N-methyl-4H-thieno[3,2-b]pyrrole-5-carboxamide | |
| | enantiomer II | |
| 422 | 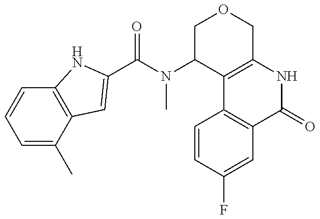 [SMILES: Cc1cccc2[nH]c(C(=O)N(C)C3COCc4[nH]c(=O)c5cc(F)ccc5c43)cc12] | 1.2 |
| | N-(8-fluoro-6-oxo-1,4,5,6-tetrahydro-2H-pyrano[3,4-c]isoquinolin-1-yl)- | |
| | N,4-dimethyl-1H-indole-2-carboxamide | |
| | enantiomer I | |
| 423 | 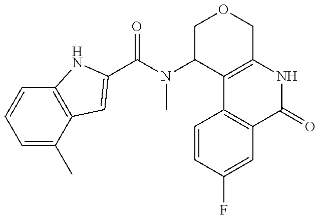 [SMILES: Cc1cccc2[nH]c(C(=O)N(C)C3COCc4[nH]c(=O)c5cc(F)ccc5c43)cc12] | 0.01 |
| | N-(8-fluoro-6-oxo-1,4,5,6-tetrahydro-2H-pyrano[3,4-c]isoquinolin-1-yl)- | |
| | N,4-dimethyl-1H-indole-2-carboxamide | |
| | enantiomer II | |
| 424 | 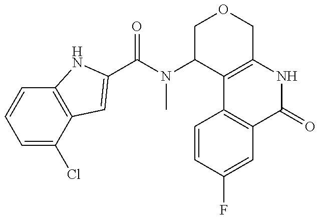 [SMILES: CN(C(=O)c1cc2c(Cl)cccc2[nH]1)C1COCc2[nH]c(=O)c3cc(F)ccc3c21] | 0.51 |
| | 4-chloro-N-(8-fluoro-6-oxo-1,4,5,6-tetrahydro-2H-pyrano[3,4-c]isoquinolin- | |
| | 1-yl)-N-methyl-1H-indole-2-carboxamide | |
| | enantiomer I | |
| 425 | 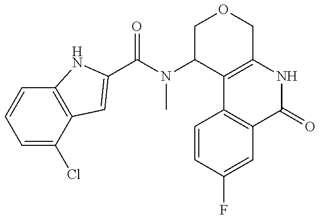 [SMILES: CN(C(=O)c1cc2c(Cl)cccc2[nH]1)C1COCc2[nH]c(=O)c3cc(F)ccc3c21] | 0.02 |
| | 4-chloro-N-(8-fluoro-6-oxo-1,4,5,6-tetrahydro-2H-pyrano[3,4-c]isoquinolin- | |
| | 1-yl)-N-methyl-1H-indole-2-carboxamide | |
| | enantiomer II | |
| 426 | 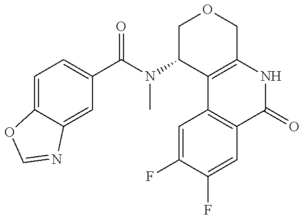 [SMILES: CN(C(=O)c1ccc2ocnc2c1)[C@@H]1COCc2[nH]c(=O)c3cc(F)c(F)cc3c21] | 0.09 |
| | (S)-N-(8,9-difluoro-6-oxo-1,4,5,6-tetrahydro-2H-pyrano[3,4-c]isoquinolin- | |
| | 1-y1)-N-methylbenzo[d]oxazole-5-carboxamide | |
| 427 | 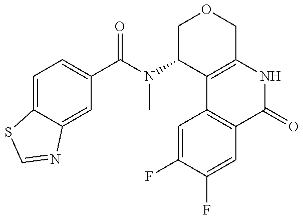 [SMILES: CN(C(=O)c1ccc2scnc2c1)[C@@H]1COCc2[nH]c(=O)c3cc(F)c(F)cc3c21] | 0.17 |
| | (S)-N-(8,9-difluoro-6-oxo-1,4,5,6-tetrahydro-2H-pyrano[3,4-c]isoquinolin- | |
| | 1-yl)-N-methylbenzo[d]thiazole-5-carboxamide | |
| 428 | 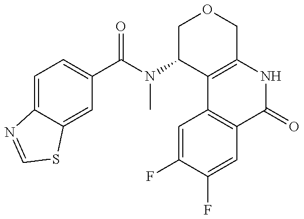 [SMILES: CN(C(=O)c1ccc2ncsc2c1)[C@@H]1COCc2[nH]c(=O)c3cc(F)c(F)cc3c21] | 0.34 |
| | (S)-N-(8,9-difluoro-6-oxo-1,4,5,6-tetrahydro-2H-pyrano[3,4-c]isoquinolin- | |
| | 1-y1)-N-methylbenzo[d]thiazole-6-carboxamide | |
| 429 | 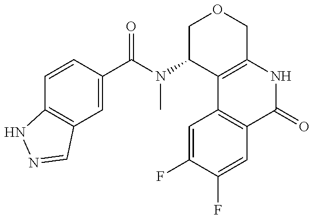 [SMILES: CN(C(=O)c1ccc2[nH]ncc2c1)[C@@H]1COCc2[nH]c(=O)c3cc(F)c(F)cc3c21] | 0.98 |
| | (S)-N-(8,9-difluoro-6-oxo-1,4,5,6-tetrahydro-2H-pyrano[3,4-c]isoquinolin- | |
| | 1-yl)-N-methyl-1H-indazole-5-carboxamide | |
| 430 | 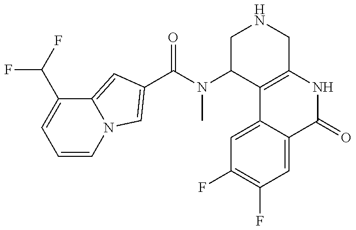 [SMILES: CN(C(=O)c1cc2c(C(F)F)cccn2c1)C1CNCc2[nH]c(=O)c3cc(F)c(F)cc3c21] | 6.3 |
| | N-(8,9-difluoro-6-oxo-1,2,3,4,5,6-hexahydrobenzo[c][1,7]naphthyridin-1- | |
| | yl)-8-(difluoromethyl)-N-methylindolizine-2-carboxamide | |
| | enantiomer I | |
| 431 | 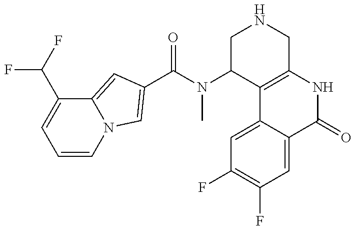 [SMILES: CN(C(=O)c1cc2c(C(F)F)cccn2c1)C1CNCc2[nH]c(=O)c3cc(F)c(F)cc3c21] | 0.01 |
| | N-(8,9-difluoro-6-oxo-1,2,3,4,5,6-hexahydrobenzo[c][1,7]naphthyridin-1- | |
| | yl)-8-(difluoromethyl)-N-methylindolizine-2-carboxamide | |
| | enantiomer II | |
| 432 | 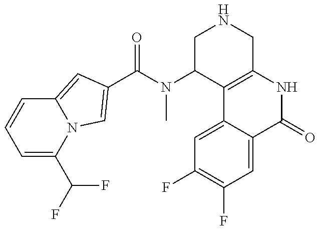 [SMILES: CN(C(=O)c1cc2cccc(C(F)F)n2c1)C1CNCc2[nH]c(=O)c3cc(F)c(F)cc3c21] | 23 |
| | N-(8,9-difluoro-6-oxo-1,2,3,4,5,6-hexahydrobenzo[c][1,7]naphthyridin-1- | |
| | y1)-5-(difluoromethyl)-N-methylindolizine-2-carboxamide | |
| | enantiomer I | |
| 433 | 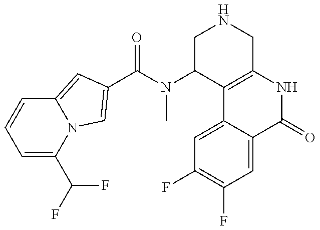 [SMILES: CN(C(=O)c1cc2cccc(C(F)F)n2c1)C1CNCc2[nH]c(=O)c3cc(F)c(F)cc3c21] | 0.05 |
| | N-(8,9-difluoro-6-oxo-1,2,3,4,5,6-hexahydrobenzo[c][1,7]naphthyridin-1- | |
| | yl)-5-(difluoromethyl)-N-methylindolizine-2-carboxamide | |
| | enantiomer II | |
| 435 | 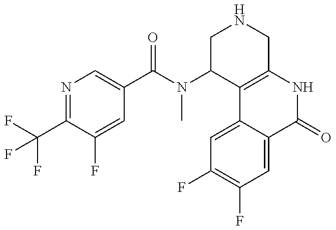 [SMILES: CN(C(=O)c1cnc(C(F)(F)F)c(F)c1)C1CNCc2[nH]c(=O)c3cc(F)c(F)cc3c21] | 0.26 |
| | N-(8,9-difluoro-6-oxo-1,2,3,4,5,6-hexahydrobenzo[c][1,7]naphthyridin-1- | |
| | y1)-5-fluoro-N-methyl-6-(trifluoromethyl)nicotinamide | |
| | enantiomer II | |
| 436 | 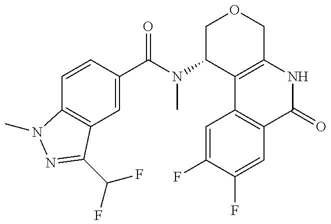 [SMILES: CN(C(=O)c1ccc2c(c1)c(C(F)F)nn2C)[C@@H]1COCc2[nH]c(=O)c3cc(F)c(F)cc3c21] | 0.10 |
| | (S)-N-(8,9-difluoro-6-oxo-1,4,5,6-tetrahydro-2H-pyrano[3,4-c]isoquinolin- | |
| | 1-yl)-3-(difluoromethyl)-N,1-dimethyl-1H-indazole-5-carboxamide | |
| 437 | 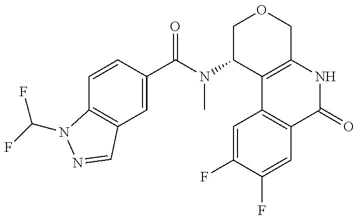 [SMILES: CN(C(=O)c1ccc2c(cnn2C(F)F)c1)[C@@H]1COCc2[nH]c(=O)c3cc(F)c(F)cc3c21] | 0.005 |
| | (S)-N-(8,9-difluoro-6-oxo-1,4,5,6-tetrahydro-2H-pyrano[3,4-c]isoquinolin- | |
| | 1-yl)-1-(difluoromethyl)-N-methyl-1H-indazole-5-carboxamide | |
| 438 | 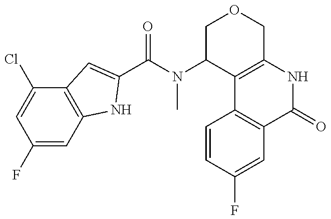 [SMILES: CN(C(=O)c1cc2c(Cl)cc(F)cc2[nH]1)C1COCc2[nH]c(=O)c3cc(F)ccc3c21] | 1.7 |
| | 4-chloro-6-fluoro-N-(8-fluoro-6-oxo-1,4,5,6-tetrahydro-2H-pyrano[3,4- | |
| | c]isoquinolin-1-yl)-N-methyl-1H-indole-2-carboxamide | |
| | enantiomer I | |
| 439 | 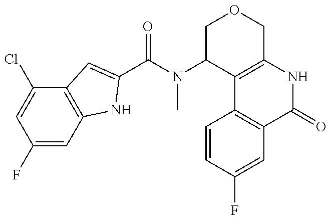 [SMILES: CN(C(=O)c1cc2c(Cl)cc(F)cc2[nH]1)C1COCc2[nH]c(=O)c3cc(F)ccc3c21] | 0.02 |
| | 4-chloro-6-fluoro-N-(8-fluoro-6-oxo-1,4,5,6-tetrahydro-2H-pyrano[3,4- | |
| | c]isoquinolin-1-yl)-N-methyl-1H-indole-2-carboxamide | |
| | enantiomer II | |
| 440 | 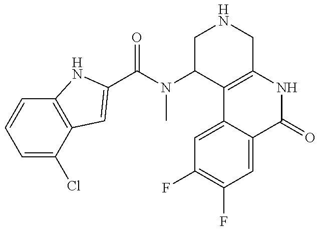 [SMILES: CN(C(=O)c1cc2c(Cl)cccc2[nH]1)C1CNCc2[nH]c(=O)c3cc(F)c(F)cc3c21] | 2.2 |
| | 4-chloro-N-(8,9-difluoro-6-oxo-1,2,3,4,5,6- | |
| | hexahydrobenzo[c][1,7]naphthyridin-1-yl)-N-methyl-1H-indole-2- | |
| | carboxamide | |
| | enantiomer I | |
| 441 | 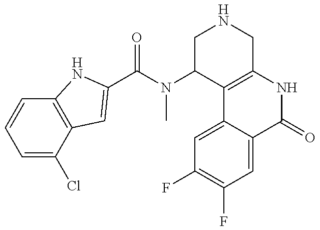 [SMILES: CN(C(=O)c1cc2c(Cl)cccc2[nH]1)C1CNCc2[nH]c(=O)c3cc(F)c(F)cc3c21] | 0.02 |
| | 4-chloro-N-(8,9-difluoro-6-oxo-1,2,3,4,5,6- | |
| | hexahydrobenzo[c][1,7]naphthyridin-1-yl)-N-methyl-1H-indole-2- | |
| | carboxamide | |
| | enantiomer II | |
| 442 | 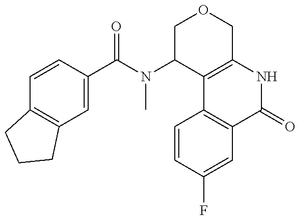 [SMILES: CN(C(=O)c1ccc2c(c1)CCC2)C1COCc2[nH]c(=O)c3cc(F)ccc3c21] | 19 |
| | N-(8-fluoro-6-oxo-1,4,5,6-tetrahydro-2H-pyrano[3,4-c]isoquinolin-1-yl)-N- | |
| | methyl-2,3-dihydro-1H-indene-5-carboxamide | |
| | enantiomer I | |
| 443 | 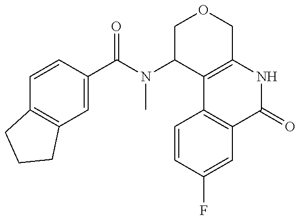 [SMILES: CN(C(=O)c1ccc2c(c1)CCC2)C1COCc2[nH]c(=O)c3cc(F)ccc3c21] | 0.64 |
| | N-(8-fluoro-6-oxo-1,4,5,6-tetrahydro-2H-pyrano[3,4-c]isoquinolin-1-yl)-N- | |
| | methyl-2,3-dihydro-1H-indene-5-carboxamide | |
| | enantiomer II | |
| 444 | 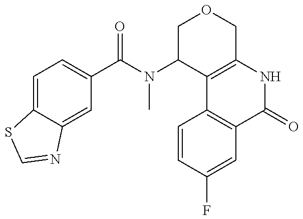 [SMILES: CN(C(=O)c1ccc2scnc2c1)C1COCc2[nH]c(=O)c3cc(F)ccc3c21] | 12.2 |
| | N-(8-fluoro-6-oxo-1,4,5,6-tetrahydro-2H-pyrano[3,4-c]isoquinolin-1-yl)-N- | |
| | methylbenzo[d]thiazole-5-carboxamide | |
| | enantiomer I | |
| 445 | 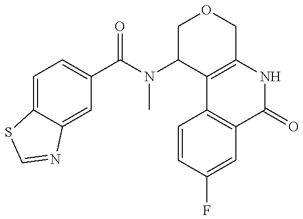 [SMILES: CN(C(=O)c1ccc2scnc2c1)C1COCc2[nH]c(=O)c3cc(F)ccc3c21] | 0.70 |
| | N-(8-fluoro-6-oxo-1,4,5,6-tetrahydro-2H-pyrano[3,4-c]isoquinolin-1-yl)-N- | |
| | methylbenzo[d]thiazole-5-carboxamide | |
| | enantiomer II | |
| 446 | 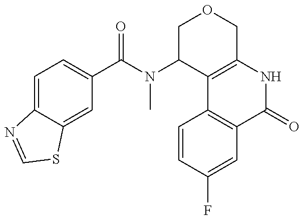 [SMILES: CN(C(=O)c1ccc2ncsc2c1)C1COCc2[nH]c(=O)c3cc(F)ccc3c21] | 16 |
| | N-(8-fluoro-6-oxo-1,4,5,6-tetrahydro-2H-pyrano[3,4-c]isoquinolin-1-yl)-N- | |
| | methylbenzo[d]thiazole-6-carboxamide | |
| | enantiomer I | |
| 447 | 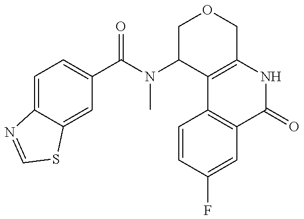 [SMILES: CN(C(=O)c1ccc2ncsc2c1)C1COCc2[nH]c(=O)c3cc(F)ccc3c21] | 0.94 |
| | N-(8-fluoro-6-oxo-1,4,5,6-tetrahydro-2H-pyrano[3,4-c]isoquinolin-1-yl)-N- | |
| | methylbenzo[d]thiazole-6-carboxamide | |
| | enantiomer II | |
| 449 | 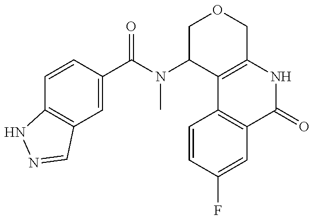 [SMILES: CN(C(=O)c1ccc2[nH]ncc2c1)C1COCc2[nH]c(=O)c3cc(F)ccc3c21] | 5.6 |
| | N-(8-fluoro-6-oxo-1,4,5,6-tetrahydro-2H-pyrano[3,4-c]isoquinolin-1-yl)-N- | |
| | methyl-1H-indazole-5-carboxamide | |
| | enantiomer II | |
| 450 | 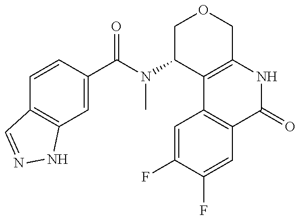 [SMILES: CN(C(=O)c1ccc2cn[nH]c2c1)[C@@H]1COCc2[nH]c(=O)c3cc(F)c(F)cc3c21] | 0.11 |
| | (S)-N-(8,9-difluoro-6-oxo-1,4,5,6-tetrahydro-2H-pyrano[3,4-c]isoquinolin- | |
| | 1-yl)-N-methyl-1H-indazole-6-carboxamide | |
| 451 | 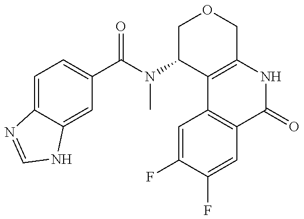 [SMILES: CN(C(=O)c1ccc2nc[nH]c2c1)[C@@H]1COCc2[nH]c(=O)c3cc(F)c(F)cc3c21] | 12.3 |
| | (S)-N-(8,9-difluoro-6-oxo-1,4,5,6-tetrahydro-2H-pyrano[3,4-c]isoquinolin- | |
| | 1-y1)-N-methyl-1H-benzo[d]imidazole-6-carboxamide | |
| 452 | 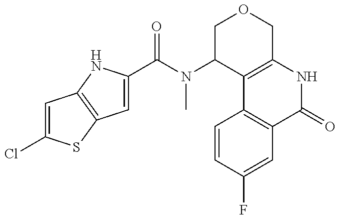 [SMILES: CN(C(=O)c1cc2sc(Cl)cc2[nH]1)C1COCc2[nH]c(=O)c3cc(F)ccc3c21] | 0.08 |
| | 2-chloro-N-(8-fluoro-6-oxo-1,4,5,6-tetrahydro-2H-pyrano[3,4-c]isoquinolin- | |
| | 1-yl)-N-methyl-4H-thieno[3,2-b]pyrrole-5-carboxamide | |
| 453 | 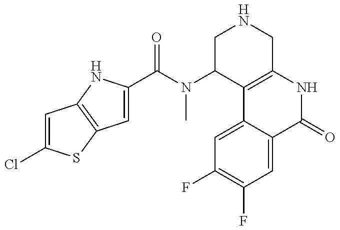 [SMILES: CN(C(=O)c1cc2sc(Cl)cc2[nH]1)C1CNCc2[nH]c(=O)c3cc(F)c(F)cc3c21] | 2.2 |
| | 2-chloro-N-(8,9-difluoro-6-oxo-1,2,3,4,5,6- | |
| | hexahydrobenzo[c][1,7]naphthyridin-1-yl)-N-methyl-4H-thieno[3,2- | |
| | b]pyrrole-5-carboxamide | |
| | enantiomer I | |
| 454 | 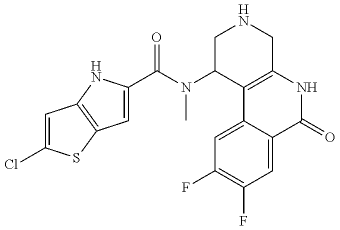 [SMILES: CN(C(=O)c1cc2sc(Cl)cc2[nH]1)C1CNCc2[nH]c(=O)c3cc(F)c(F)cc3c21] | 0.02 |
| | 2-chloro-N-(8,9-difluoro-6-oxo-1,2,3,4,5,6- | |
| | hexahydrobenzo[c][1,7]naphthyridin-1-yl)-N-methyl-4H-thieno[3,2- | |
| | b]pyrrole-5-carboxamide | |
| | enantiomer II | |
| 455 | 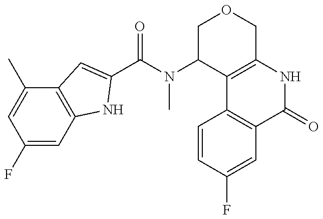 [SMILES: Cc1cc(F)cc2[nH]c(C(=O)N(C)C3COCc4[nH]c(=O)c5cc(F)ccc5c43)cc12] | 0.02 |
| | 6-fluoro-N-(8-fluoro-6-oxo-1,4,5,6-tetrahydro-2H-pyrano[3,4-c]isoquinolin- | |
| | 1-yl)-N,4-dimethyl-1H-indole-2-carboxamide | |
| 456 | 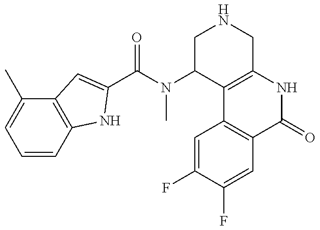 [SMILES: Cc1cccc2[nH]c(C(=O)N(C)C3CNCc4[nH]c(=O)c5cc(F)c(F)cc5c43)cc12] | 2.2 |
| | N-(8,9-difluoro-6-oxo-1,2,3,4,5,6-hexahydrobenzo[c][1,7]naphthyridin-1- | |
| | yl)-N,4-dimethyl-1H-indole-2-carboxamide | |
| | enantiomer I | |
| 457 | 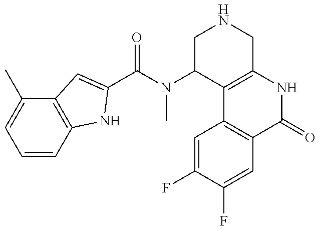 [SMILES: Cc1cccc2[nH]c(C(=O)N(C)C3CNCc4[nH]c(=O)c5cc(F)c(F)cc5c43)cc12] | 0.01 |
| | N-(8,9-difluoro-6-oxo-1,2,3,4,5,6-hexahydrobenzo[c][1,7]naphthyridin-1- | |
| | yl)-N,4-dimethyl-1H-indole-2-carboxamide | |
| | enantiomer II | |
| 458 | 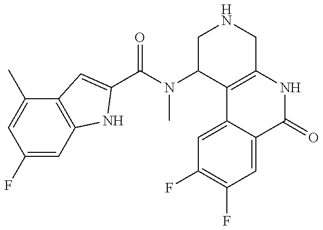 [SMILES: Cc1cc(F)cc2[nH]c(C(=O)N(C)C3CNCc4[nH]c(=O)c5cc(F)c(F)cc5c43)cc12] | 6.6 |
| | N-(8,9-difluoro-6-oxo-1,2,3,4,5,6-hexahydrobenzo[c][1,7]naphthyridin-1- | |
| | yl)-6-fluoro-N,4-dimethyl-1H-indole-2-carboxamide | |
| | enantiomer I | |
| 459 | 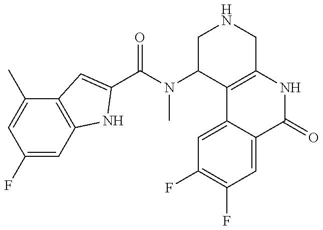 [SMILES: Cc1cc(F)cc2[nH]c(C(=O)N(C)C3CNCc4[nH]c(=O)c5cc(F)c(F)cc5c43)cc12] | 0.01 |
| | N-(8,9-difluoro-6-oxo-1,2,3,4,5,6-hexahydrobenzo[c][1,7]naphthyridin-1- | |
| | yl)-6-fluoro-N,4-dimethyl-1H-indole-2-carboxamide | |
| | enantiomer II | |
| 460 | 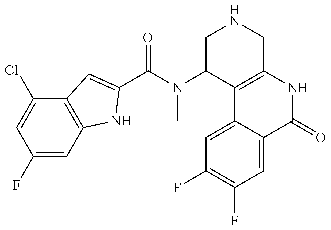 [SMILES: CN(C(=O)c1cc2c(Cl)cc(F)cc2[nH]1)C1CNCc2[nH]c(=O)c3cc(F)c(F)cc3c21] | 11.2 |
| | 4-chloro-N-(8,9-difluoro-6-oxo-1,2,3,4,5,6- | |
| | hexahydrobenzo[c][1,7]naphthyridin-1-yl)-6-fluoro-N-methyl-1H-indole-2- | |
| | carboxamide | |
| | enantiomer I | |
| 461 | 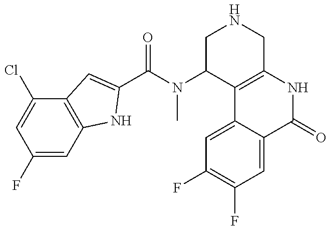 [SMILES: CN(C(=O)c1cc2c(Cl)cc(F)cc2[nH]1)C1CNCc2[nH]c(=O)c3cc(F)c(F)cc3c21] | 0.03 |
| | 4-chloro-N-(8,9-difluoro-6-oxo-1,2,3,4,5,6- | |
| | hexahydrobenzo[c][1,7]naphthyridin-1-yl)-6-fluoro-N-methyl-1H-indole-2- | |
| | carboxamide | |
| | enantiomer II | |
| 463 | 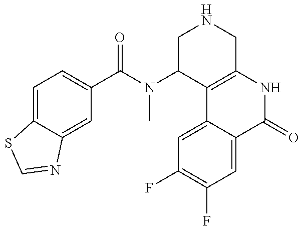 [SMILES: CN(C(=O)c1ccc2scnc2c1)C1CNCc2[nH]c(=O)c3cc(F)c(F)cc3c21] | 2.0 |
| | N-(8,9-difluoro-6-oxo-1,2,3,4,5,6-hexahydrobenzo[c][1,7]naphthyridin-1- | |
| | yl)-N-methylbenzo[d]thiazole-5-carboxamide | |
| | enantiomer II | |
| 464 | 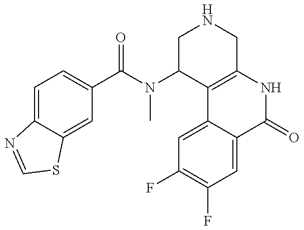 [SMILES: CN(C(=O)c1ccc2ncsc2c1)C1CNCc2[nH]c(=O)c3cc(F)c(F)cc3c21] | 25 |
| | N-(8,9-difluoro-6-oxo-1,2,3,4,5,6-hexahydrobenzo[c][1,7]naphthyridin-1- | |
| | yl)-N-methylbenzo[d]thiazole-6-carboxamide | |
| | enantiomer I | |
| 465 | 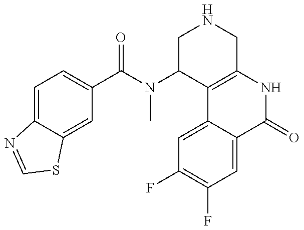 [SMILES: CN(C(=O)c1ccc2ncsc2c1)C1CNCc2[nH]c(=O)c3cc(F)c(F)cc3c21] | 3.2 |
| | N-(8,9-difluoro-6-oxo-1,2,3,4,5,6-hexahydrobenzo[c][1,7]naphthyridin-1- | |
| | yl)-N-methylbenzo[d]thiazole-6-carboxamide | |
| | enantiomer II | |
| 466 | 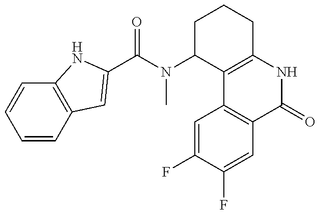 [SMILES: CN(C(=O)c1cc2ccccc2[nH]1)C1CCCc2[nH]c(=O)c3cc(F)c(F)cc3c21] | 2.1 |
| | N-(8,9-difluoro-6-oxo-1,2,3,4,5,6-hexahydrophenanthridin-1-yl)-N-methyl- | |
| | 1H-indole-2-carboxamide | |
| | enantiomer I | |
| 467 | 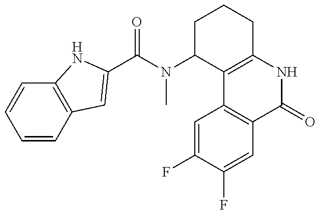 [SMILES: CN(C(=O)c1cc2ccccc2[nH]1)C1CCCc2[nH]c(=O)c3cc(F)c(F)cc3c21] | 0.01 |
| | N-(8,9-difluoro-6-oxo-1,2,3,4,5,6-hexahydrophenanthridin-1-yl)-N-methyl- | |
| | 1H-indole-2-carboxamide | |
| | enantiomer II | |
| 468 | 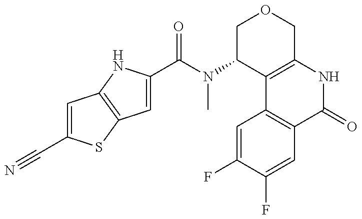 [SMILES: CN(C(=O)c1cc2sc(C#N)cc2[nH]1)[C@@H]1COCc2[nH]c(=O)c3cc(F)c(F)cc3c21] | 0.005 |
| | (S)-2-cyano-N-(8,9-difluoro-6-oxo-1,4,5,6-tetrahydro-2H-pyrano[3,4- | |
| | c]isoquinolin-1-yl)-N-methyl-4H-thieno[3,2-b]pyrrole-5-carboxamide | |
| 469 | 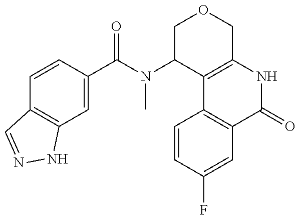 [SMILES: CN(C(=O)c1ccc2cn[nH]c2c1)C1COCc2[nH]c(=O)c3cc(F)ccc3c21] | 2.2 |
| | N-(8-fluoro-6-oxo-1,4,5,6-tetrahydro-2H-pyrano[3,4-c]isoquinolin-1-yl)-N- | |
| | methyl-1H-indazole-6-carboxamide | |
| | enantiomer I | |
| 470 | 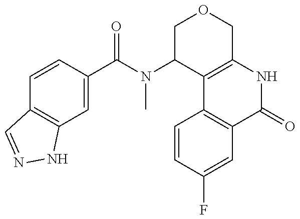 [SMILES: CN(C(=O)c1ccc2cn[nH]c2c1)C1COCc2[nH]c(=O)c3cc(F)ccc3c21] | 1.1 |
| | N-(8-fluoro-6-oxo-1,4,5,6-tetrahydro-2H-pyrano[3,4-c]isoquinolin-1-yl)-N- | |
| | methyl-1H-indazole-6-carboxamide | |
| | enantiomer II | |
| 471 | 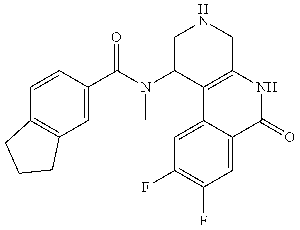 [SMILES: CN(C(=O)c1ccc2c(c1)CCC2)C1CNCc2[nH]c(=O)c3cc(F)c(F)cc3c21] | 8.7 |
| | N-(8,9-difluoro-6-oxo-1,2,3,4,5,6-hexahydrobenzo[c][1,7]naphthyridin-1- | |
| | yl)-N-methyl-2,3-dihydro-1H-indene-5-carboxamide | |
| | enantiomer I | |
| 472 | 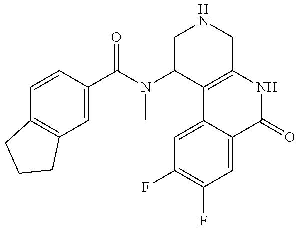 [SMILES: CN(C(=O)c1ccc2c(c1)CCC2)C1CNCc2[nH]c(=O)c3cc(F)c(F)cc3c21] | 0.04 |
| | N-(8,9-difluoro-6-oxo-1,2,3,4,5,6-hexahydrobenzo[c][1,7]naphthyridin-1- | |
| | yl)-N-methyl-2,3-dihydro-1H-indene-5-carboxamide | |
| | enantiomer II | |
| 474 | 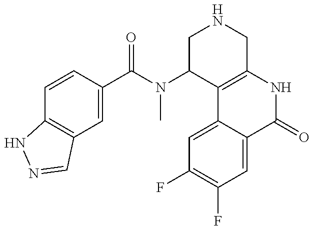 [SMILES: CN(C(=O)c1ccc2[nH]ncc2c1)C1CNCc2[nH]c(=O)c3cc(F)c(F)cc3c21] | 3.8 |
| | N-(8,9-difluoro-6-oxo-1,2,3,4,5,6-hexahydrobenzo[c][1,7]naphthyridin-1- | |
| | yl)-N-methyl-1H-indazole-5-carboxamide | |
| | enantiomer II | |
| 475 | 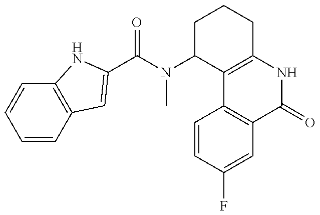 [SMILES: CN(C(=O)c1cc2ccccc2[nH]1)C1CCCc2[nH]c(=O)c3cc(F)ccc3c21] | 1.8 |
| | N-(8-fluoro-6-oxo-1,2,3,4,5,6-hexahydrophenanthridin-1-yl)-N-methyl-1H- | |
| | indole-2-carboxamide | |
| | enantiomer I | |
| 476 | 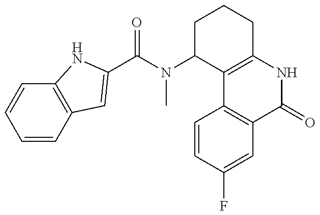 [SMILES: CN(C(=O)c1cc2ccccc2[nH]1)C1CCCc2[nH]c(=O)c3cc(F)ccc3c21] | 0.02 |
| | N-(8-fluoro-6-oxo-1,2,3,4,5,6-hexahydrophenanthridin-1-yl)-N-methyl-1H- | |
| | indole-2-carboxamide | |
| | enantiomer II | |
| 477 | 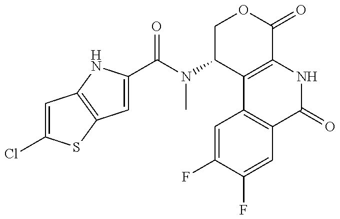 [SMILES: CN(C(=O)c1cc2sc(Cl)cc2[nH]1)[C@@H]1COC(=O)c2[nH]c(=O)c3cc(F)c(F)cc3c21] | 0.02 |
| | (S)-2-chloro-N-(8,9-difluoro-4,6-dioxo-1,4,5,6-tetrahydro-2H-pyrano[3,4- | |
| | c]isoquinolin-1-yl)-N-methyl-4H-thieno[3,2-b]pyrrole-5-carboxamide | |
| 478 | 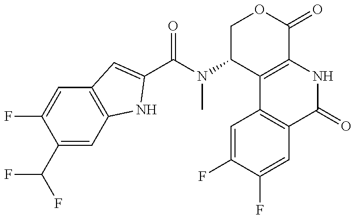 [SMILES: CN(C(=O)c1cc2cc(F)c(C(F)F)cc2[nH]1)[C@@H]1COC(=O)c2[nH]c(=O)c3cc(F)c(F)cc3c21] | 0.01 |
| | (S)-N-(8,9-difluoro-4,6-dioxo-1,4,5,6-tetrahydro-2H-pyrano[3,4- | |
| | c]isoquinolin-1-yl)-6-(difluoromethyl)-5-fluoro-N-methyl-1H-indole-2- | |
| | carboxamide | |
| 479 | 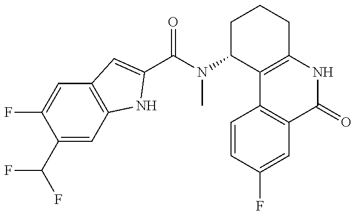 [SMILES: CN(C(=O)c1cc2cc(F)c(C(F)F)cc2[nH]1)[C@@H]1CCCc2[nH]c(=O)c3cc(F)ccc3c21] | 0.04 |
| | (R)-6-(difluoromethyl)-5-fluoro-N-(8-fluoro-6-oxo-1,2,3,4,5,6- | |
| | hexahydrophenanthridin-1-yl)-N-methyl-1H-indole-2-carboxamide | |
| 480 | 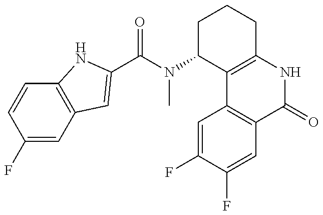 [SMILES: CN(C(=O)c1cc2cc(F)ccc2[nH]1)[C@@H]1CCCc2[nH]c(=O)c3cc(F)c(F)cc3c21] | 0.01 |
| | (R)-N-(8,9-difluoro-6-oxo-1,2,3,4,5,6-hexahydrophenanthridin-1-yl)-5- | |
| | fluoro-N-methyl-1H-indole-2-carboxamide | |
| 481 | 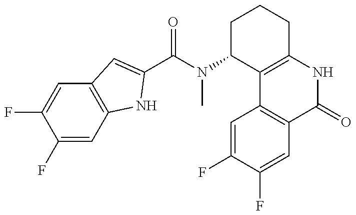 [SMILES: CN(C(=O)c1cc2cc(F)c(F)cc2[nH]1)[C@@H]1CCCc2[nH]c(=O)c3cc(F)c(F)cc3c21] | 0.01 |
| | (R)-N-(8,9-difluoro-6-oxo-1,2,3,4,5,6-hexahydrophenanthridin-1-yl)-5,6- | |
| | difluoro-N-methyl-1H-indole-2-carboxamide | |
| 482 | 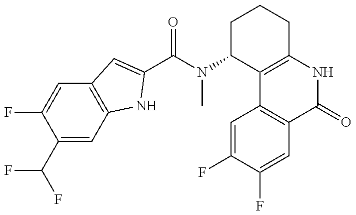 [SMILES: CN(C(=O)c1cc2cc(F)c(C(F)F)cc2[nH]1)[C@@H]1CCCc2[nH]c(=O)c3cc(F)c(F)cc3c21] | 0.01 |
| | (R)-N-(8,9-difluoro-6-oxo-1,2,3,4,5,6-hexahydrophenanthridin-1-yl)-6- | |
| | (difluoromethyl)-5-fluoro-N-methyl-1H-indole-2-carboxamide | |
| 483 | 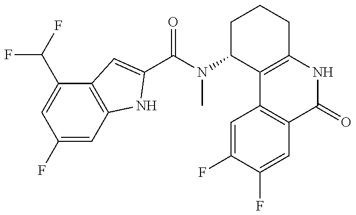 [SMILES: CN(C(=O)c1cc2c(C(F)F)cc(F)cc2[nH]1)[C@@H]1CCCc2[nH]c(=O)c3cc(F)c(F)cc3c21] | 0.01 |
| | (R)-N-(8,9-difluoro-6-oxo-1,2,3,4,5,6-hexahydrophenanthridin-1-yl)-4- | |
| | (difluoromethyl)-6-fluoro-N-methyl-1H-indole-2-carboxamide | |
| 484 | 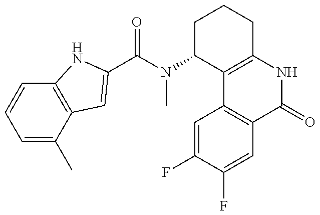 [SMILES: Cc1cccc2[nH]c(C(=O)N(C)[C@@H]3CCCc4[nH]c(=O)c5cc(F)c(F)cc5c43)cc12] | 0.01 |
| | (R)-N-(8,9-difluoro-6-oxo-1,2,3,4,5,6-hexahydrophenanthridin-1-yl)-N,4- | |
| | dimethyl-1H-indole-2-carboxamide | |
| 485 | 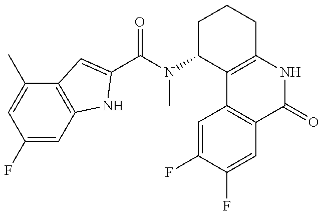 [SMILES: Cc1cc(F)cc2[nH]c(C(=O)N(C)[C@@H]3CCCc4[nH]c(=O)c5cc(F)c(F)cc5c43)cc12] | 0.01 |
| | (R)-N-(8,9-difluoro-6-oxo-1,2,3,4,5,6-hexahydrophenanthridin-1-yl)-6- | |
| | fluoro-N,4-dimethyl-1H-indole-2-carboxamide | |
| 486 | 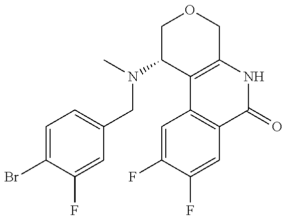 [SMILES: CN(Cc1ccc(Br)c(F)c1)[C@@H]1COCc2[nH]c(=O)c3cc(F)c(F)cc3c21] | 8.28 |
| | (S)-1-((4-bromo-3-fluorobenzyl)(methyl)amino)-8,9-difluoro-1,5-dihydro- | |
| | 2H-pyrano[3,4-c]isoquinolin-6(4H)-one | |
| 487 | 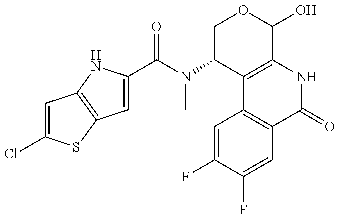 [SMILES: CN(C(=O)c1cc2sc(Cl)cc2[nH]1)[C@@H]1COC(O)c2[nH]c(=O)c3cc(F)c(F)cc3c21] | 0.02 |
| | 2-chloro-N-((1S)-8,9-difluoro-4-hydroxy-6-oxo-1,4,5,6-tetrahydro-2H- | |
| | pyrano[3,4-c]isoquinolin-1-yl)-N-methyl-4H-thieno[3,2-b]pyrrole-5- | |
| | carboxamide | |
| | ~5:1 ratio of epimers at anomeric carbon | |
| 488 | 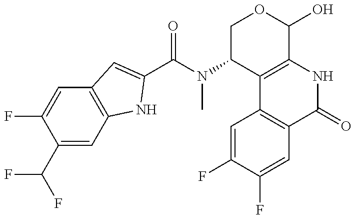 [SMILES: CN(C(=O)c1cc2cc(F)c(C(F)F)cc2[nH]1)[C@@H]1COC(O)c2[nH]c(=O)c3cc(F)c(F)cc3c21] | 0.01 |
| | N-((1S)-8,9-difluoro-4-hydroxy-6-oxo-1,4,5,6-tetrahydro-2H-pyrano[3,4- | |
| | c]isoquinolin-1-yl)-6-(difluoromethyl)-5-fluoro-N-methyl-1H-indole-2- | |
| | carboxamide | |
| | ~6:1 ratio of epimers at anomeric carbon | |
| 489 | 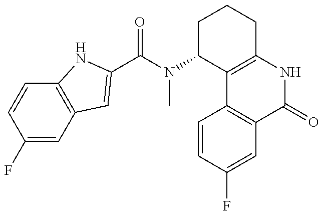 [SMILES: CN(C(=O)c1cc2cc(F)ccc2[nH]1)[C@@H]1CCCc2[nH]c(=O)c3cc(F)ccc3c21] | 0.01 |
| | (R)-5-fluoro-N-(8-fluoro-6-oxo-1,2,3,4,5,6-hexahydrophenanthridin-1-yl)- | |
| | N-methyl-1H-indole-2-carboxamide | |
| 490 | 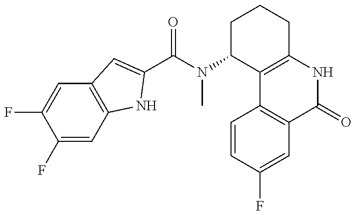 [SMILES: CN(C(=O)c1cc2cc(F)c(F)cc2[nH]1)[C@@H]1CCCc2[nH]c(=O)c3cc(F)ccc3c21] | 0.02 |
| | (R)-5,6-difluoro-N-(8-fluoro-6-oxo-1,2,3,4,5,6-hexahydrophenanthridin-1- | |
| | yl)-N-methyl-1H-indole-2-carboxamide | |
| 491 | 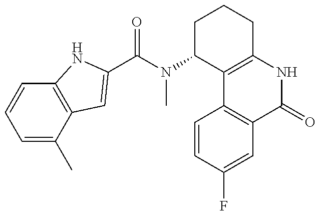 [SMILES: Cc1cccc2[nH]c(C(=O)N(C)[C@@H]3CCCc4[nH]c(=O)c5cc(F)ccc5c43)cc12] | 0.02 |
| | (R)-N-(8-fluoro-6-oxo-1,2,3,4,5,6-hexahydrophenanthridin-1-y1)-N,4- | |
| | dimethyl-1H-indole-2-carboxamide | |
| 492 | 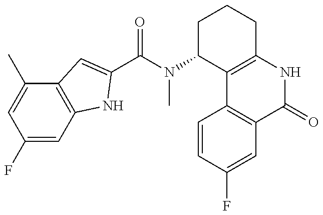 [SMILES: Cc1cc(F)cc2[nH]c(C(=O)N(C)[C@@H]3CCCc4[nH]c(=O)c5cc(F)ccc5c43)cc12] | 0.03 |
| | (R)-6-fluoro-N-(8-fluoro-6-oxo-1,2,3,4,5,6-hexahydrophenanthridin-1-y1)- | |
| | N,4-dimethyl-1H-indole-2-carboxamide | |
| 493 | 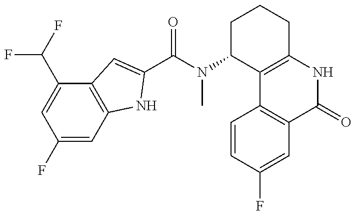 [SMILES: CN(C(=O)c1cc2c(C(F)F)cc(F)cc2[nH]1)[C@@H]1CCCc2[nH]c(=O)c3cc(F)ccc3c21] | 0.01 |
| | (R)-4-(difluoromethyl)-6-fluoro-N-(8-fluoro-6-oxo-1,2,3,4,5,6- | |
| | hexahydrophenanthridin-1-y1)-N-methyl-1H-indole-2-carboxamide | |
| 494 | 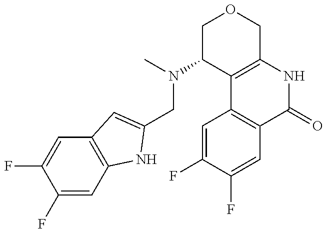 [SMILES: CN(Cc1cc2cc(F)c(F)cc2[nH]1)[C@@H]1COCc2[nH]c(=O)c3cc(F)c(F)cc3c21] | 1.5 |
| | (S)-1-(((5,6-difluoro-1H-indol-2-yl)methyl)(methyl)amino)-8,9-difluoro-1,5- | |
| | dihydro-2H-pyrano[3,4-c]isoquinolin-6(4H)-one | |
| 495 | 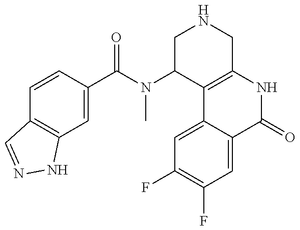 [SMILES: CN(C(=O)c1ccc2cn[nH]c2c1)C1CNCc2[nH]c(=O)c3cc(F)c(F)cc3c21] | >25 |
| | N-(8,9-difluoro-6-oxo-1,2,3,4,5,6-hexahydrobenzo[c][1,7]naphthyridin-1- | |
| | yl)-N-methyl-1H-indazole-6-carboxamide | |
| | enantiomer I | |
| 496 | 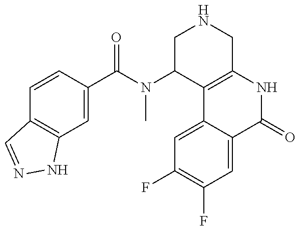 [SMILES: CN(C(=O)c1ccc2cn[nH]c2c1)C1CNCc2[nH]c(=O)c3cc(F)c(F)cc3c21] | 4.2 |
| | N-(8,9-difluoro-6-oxo-1,2,3,4,5,6-hexahydrobenzo[c][1,7]naphthyridin-1- | |
| | yl)-N-methyl-1H-indazole-6-carboxamide | |
| | enantiomer II | |
| 497 | 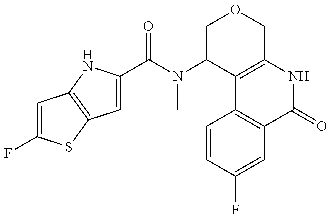 [SMILES: CN(C(=O)c1cc2sc(F)cc2[nH]1)C1COCc2[nH]c(=O)c3cc(F)ccc3c21] | 1.2 |
| | 2-fluoro-N-(8-fluoro-6-oxo-1,4,5,6-tetrahydro-2H-pyrano[3,4-c]isoquinolin- | |
| | 1-yl)-N-methyl-4H-thieno[3,2-b]pyrrole-5-carboxamide | |
| | enantiomer I | |
| 498 | 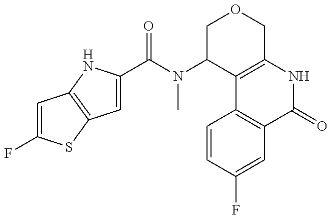 [SMILES: CN(C(=O)c1cc2sc(F)cc2[nH]1)C1COCc2[nH]c(=O)c3cc(F)ccc3c21] | 0.08 |
| | 2-fluoro-N-(8-fluoro-6-oxo-1,4,5,6-tetrahydro-2H-pyrano[3,4-c]isoquinolin- | |
| | 1-yl)-N-methyl-4H-thieno[3,2-b]pyrrole-5-carboxamide | |
| | enantiomer II | |
| 499 | 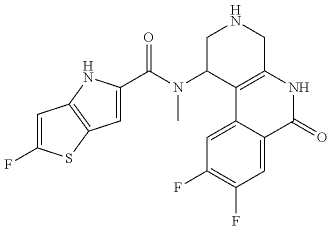 [SMILES: CN(C(=O)c1cc2sc(F)cc2[nH]1)C1CNCc2[nH]c(=O)c3cc(F)c(F)cc3c21] | 20 |
| | N-(8,9-difluoro-6-oxo-1,2,3,4,5,6-hexahydrobenzo[c][1,7]naphthyridin-1- | |
| | y1)-2-fluoro-N-methyl-4H-thieno[3,2-b]pyrrole-5-carboxamide | |
| | enantiomer I | |
| 500 | 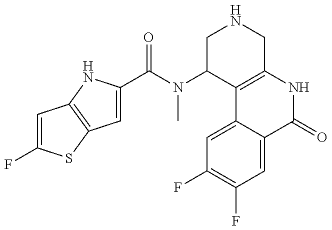 [SMILES: CN(C(=O)c1cc2sc(F)cc2[nH]1)C1CNCc2[nH]c(=O)c3cc(F)c(F)cc3c21] | 0.03 |
| | N-(8,9-difluoro-6-oxo-1,2,3,4,5,6-hexahydrobenzo[c][1,7]naphthyridin-1- | |
| | yl)-2-fluoro-N-methyl-4H-thieno[3,2-b]pyrrole-5-carboxamide | |
| | enantiomer II | |
| 501 | 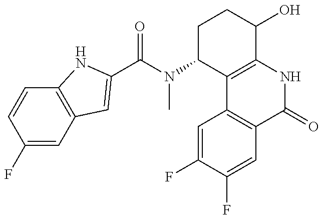 [SMILES: CN(C(=O)c1cc2cc(F)ccc2[nH]1)[C@@H]1CCC(O)c2[nH]c(=O)c3cc(F)c(F)cc3c21] | 0.01 |
| | N-((1R)-8,9-difluoro-4-hydroxy-6-oxo-1,2,3,4,5,6-hexahydrophenanthridin- | |
| | 1-y1)-5-fluoro-N-methyl-1H-indole-2-carboxamide | |
| | diastereomer I | |
| 502 | 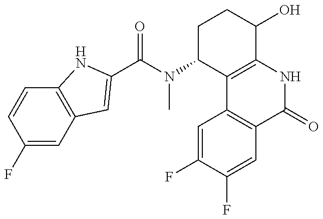 [SMILES: CN(C(=O)c1cc2cc(F)ccc2[nH]1)[C@@H]1CCC(O)c2[nH]c(=O)c3cc(F)c(F)cc3c21] | 0.03 |
| | N-((1R)-8,9-difluoro-4-hydroxy-6-oxo-1,2,3,4,5,6-hexahydrophenanthridin- | |
| | 1-yl)-5-fluoro-N-methyl-1H-indole-2-carboxamide | |
| | diastereomer II | |
| 503 | 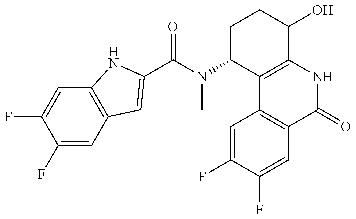 [SMILES: CN(C(=O)c1cc2cc(F)c(F)cc2[nH]1)[C@@H]1CCC(O)c2[nH]c(=O)c3cc(F)c(F)cc3c21] | 0.02 |
| | N-((1R)-8,9-difluoro-4-hydroxy-6-oxo-1,2,3,4,5,6-hexahydrophenanthridin- | |
| | 1-yl)-5,6-difluoro-N-methyl-1H-indole-2-carboxamide | |
| | ~6:1 ratio of epimers at anomeric carbon | |
| 504 | 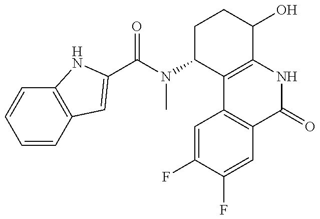 [SMILES: CN(C(=O)c1cc2ccccc2[nH]1)[C@@H]1CCC(O)c2[nH]c(=O)c3cc(F)c(F)cc3c21] | 0.58 |
| | N-((1R)-8,9-difluoro-4-hydroxy-6-oxo-1,2,3,4,5,6-hexahydrophenanthridin- | |
| | 1-yl)-N-methyl-1H-indole-2-carboxamide | |
| | diastereomer I | |
| 505 | 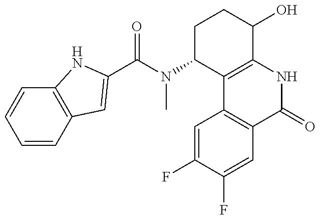 [SMILES: CN(C(=O)c1cc2ccccc2[nH]1)[C@@H]1CCC(O)c2[nH]c(=O)c3cc(F)c(F)cc3c21] | 0.05 |
| | N-((1R)-8,9-difluoro-4-hydroxy-6-oxo-1,2,3,4,5,6-hexahydrophenanthridin- | |
| | 1-yl)-N-methyl-1H-indole-2-carboxamide | |
| | diastereomer II | |
| 506 | 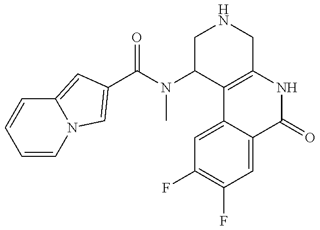 [SMILES: CN(C(=O)c1cc2ccccn2c1)C1CNCc2[nH]c(=O)c3cc(F)c(F)cc3c21] | 22 |
| | N-(8,9-difluoro-6-oxo-1,2,3,4,5,6- | |
| | hexahydrobenzo[c][1,7]naphthyridin-1-yl)-N-methylindolizine-2- | |
| | carboxamide | |
| | enantiomer I | |
| 507 | 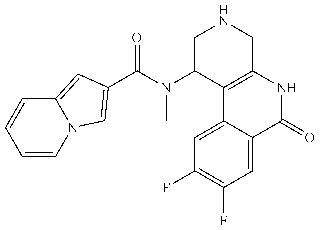 [SMILES: CN(C(=O)c1cc2ccccn2c1)C1CNCc2[nH]c(=O)c3cc(F)c(F)cc3c21] | 0.06 |
| | N-(8,9-difluoro-6-oxo-1,2,3,4,5,6- | |
| | hexahydrobenzo[c][1,7]naphthyridin-1-yl)-N-methylindolizine-2- | |
| | carboxamide | |
| | enantiomer II | |
| 508 | 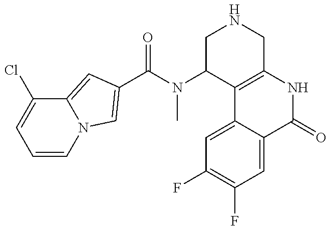 [SMILES: CN(C(=O)c1cc2c(Cl)cccn2c1)C1CNCc2[nH]c(=O)c3cc(F)c(F)cc3c21] | 11.6 |
| | 8-chloro-N-(8,9-difluoro-6-oxo-1,2,3,4,5,6- | |
| | hexahydrobenzo[c][1,7]naphthyridin-1-yl)-N-methylindolizine-2- | |
| | carboxamide | |
| | enantiomer I | |
| 509 | 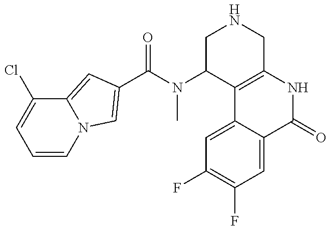 [SMILES: CN(C(=O)c1cc2c(Cl)cccn2c1)C1CNCc2[nH]c(=O)c3cc(F)c(F)cc3c21] | 0.01 |
| | 8-chloro-N-(8,9-difluoro-6-oxo-1,2,3,4,5,6- | |
| | hexahydrobenzo[c][1,7]naphthyridin-1-yl)-N-methylindolizine-2- | |
| | carboxamide enantiomer II | |
| 512 | 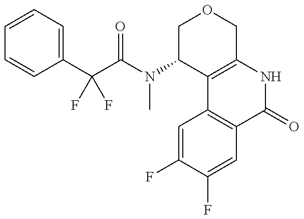 [SMILES: CN(C(=O)C(F)(F)c1ccccc1)[C@@H]1COCc2[nH]c(=O)c3cc(F)c(F)cc3c21] | 25 |
| | (S)-N-(8,9-Difluoro-6-oxo-1,4,5,6-tetrahydro-2H-pyrano[3,4- | |
| | c]isoquinolin-1-yl)-2,2-difluoro-N-methyl-2-phenylacetamide | |
| 514 | 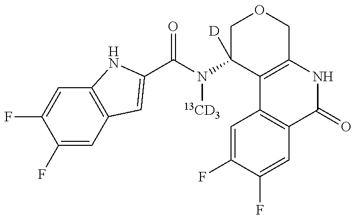 | 0.008 |
| | (S)-N-(8,9-Difluoro-6-oxo-1,4,5,6-tetrahydro-2H-pyrano[3,4- | |
| | c]isoquinolin-1-y1-1-d)-5,6-difluoro-N-(methyl-13C-d3)-1H-indole-2- | |
| | carboxamide | |
| 515 | 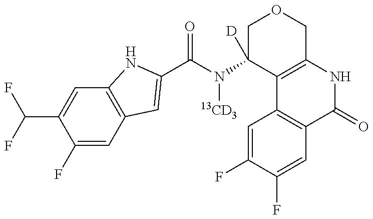 | 0.01 |
| | (S)-N-(8,9-Difluoro-6-oxo-1,4,5,6-tetrahydro-2H-pyrano[3,4- | |
| | c]isoquinolin-1-yl-1-d)-6-(difluoromethyl)-5-fluoro-N-(methyl-13C- | |
| | d3)-1H-indole-2-carboxamide | |
| 516 | 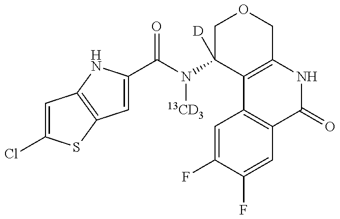 | 0.01 |
| | (S)-2-Chloro-N-(8,9-difluoro-6-oxo-1,4,5,6-tetrahydro-2H- | |
| | pyrano[3,4-c]isoquinolin-1-y1-1-d)-N-(methyl-13C-d3)-4H- | |
| | thieno[3,2-b]pyrrole-5-carboxamide | |

### Enumerated Embodiments

The following exemplary embodiments are provided, the numbering of which is not to be construed as designating levels of importance:

Embodiment 1 provides compound of formula (I), or a salt, solvate, prodrug, stereoisomer, tautomer, or isotopically labelled derivative thereof, or any mixtures thereof:

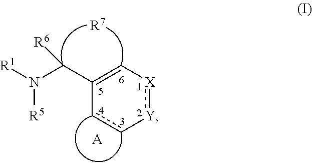

wherein:
   X, Y, and the bond between X and Y are such that:
      X is NR⁸, Y is C(═O), and the bond between X and Y is a single bond, or
      X is N, Y is CR¹¹, and the bond between X and Y is a double bond;
   ring A is selected from the group consisting of:

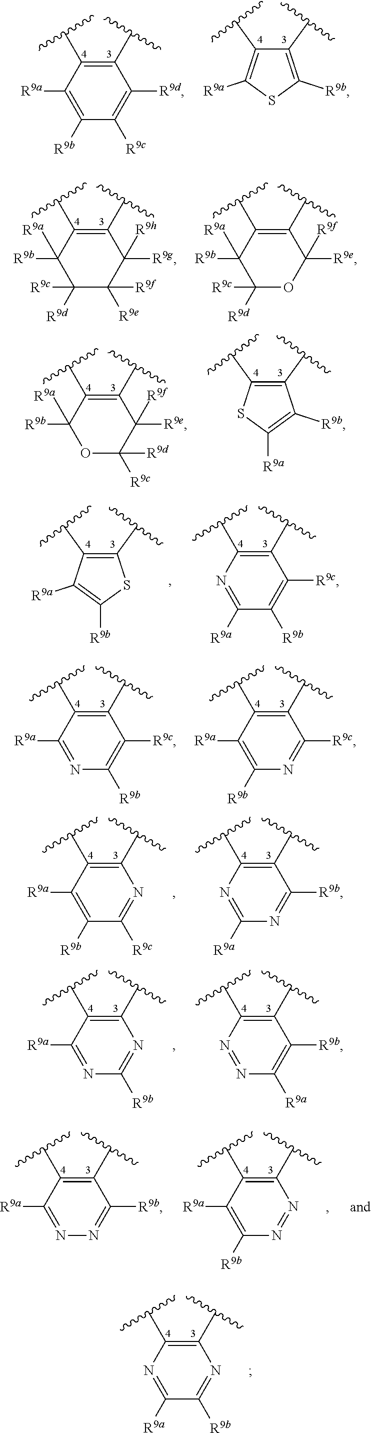

R¹ is selected from the group consisting of R²C(═O)—, R²S(═O)₂—, and aminoacyl;
R² is selected from the group consisting of:

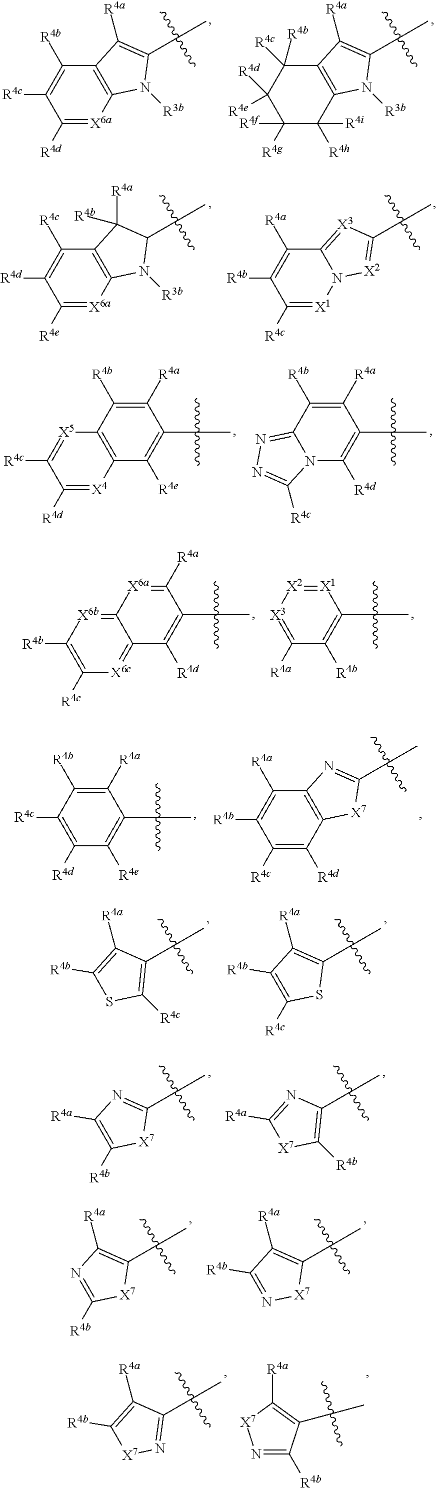

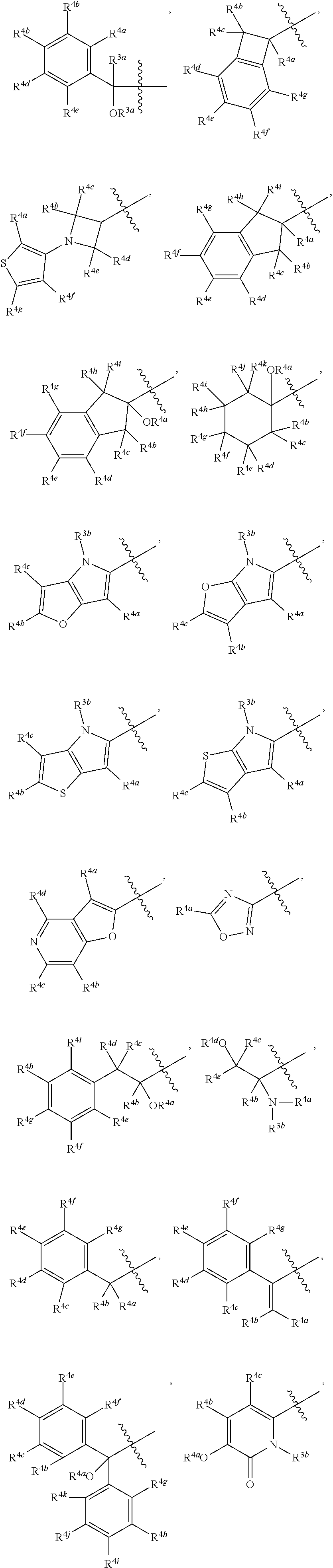

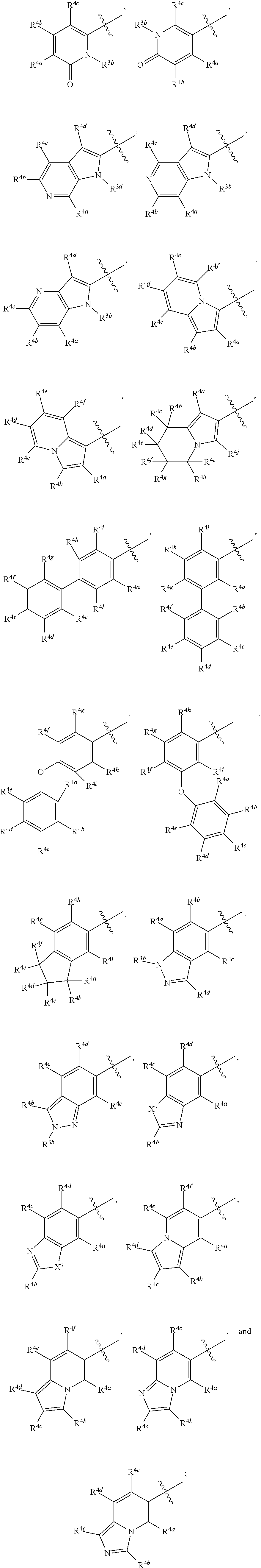

one of the following applies:
   (i) X¹ is N, X² is C(R^{4f}), and X³ is C(R^{4g});
   (ii) X² is N, X¹ is C(R^{4f}), and X³ is C(R^{4g});
   (iii) X³ is N, X⁴ is C(R^{4f}), and X² is C(R⁴R);
   (v) X¹ is C(R^{4f}), X² is C(R^{4g}), and X³ is C(R^{4h});
one of the following applies:
   (i) X⁴ is N and X⁵ is C(R^{4e}); or
   (ii) X⁵ is N and X⁴ is C(R^{4e});
each occurrence of X^{6a} is independently N or C(R^{4f});
each occurrence of X^{6b} is independently N or C(R^{4g});
each occurrence of X^{6c} is independently N or C(R^{4h});
each occurrence of X⁷ is independently S, O, or NR^{3a};
each occurrence of R³ is independently selected from the group consisting of H, optionally substituted C₁-C₆ alkyl, and optionally substituted C₃-C₈ cycloalkyl;
each occurrence of R^{3b} is independently selected from the group consisting of H, optionally substituted C₁-C₆ alkyl, and optionally substituted C₃-C₈ cycloalkyl;
each occurrence of R^{4a}, R^{4b}, R^{4c}, R^{4d}, R^{4e}, R^{4f}, R^{4g}, R^{4h}, R⁴ⁱ, R^{4j} and R^{4k} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, optionally substituted phenyl, optionally substituted heterocyclyl, optionally substituted heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R⁵ is independently selected from the group consisting of H, C₁-C₆ alkyl, and C₃-C₈ cycloalkyl, wherein the alkyl or cycloalkyl is optionally substituted with at least one selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, optionally substituted phenyl, optionally substituted heteroaryl, optionally substituted heterocyclyl, —C(═O)OR¹⁰, —OC(═O)R¹⁰, —SR¹⁰, —S(═O)R¹⁰, —S(═O)₂R¹⁰, —S(═O)₂NR¹⁰R¹⁰, —N(R¹⁰)S(═O)₂R¹⁰, —N(R¹⁰)C(═O)R¹⁰, —C(═O)NR¹⁰R¹⁰, and —NR¹⁰R¹⁰;
each occurrence of R⁶ is independently selected from the group consisting of H and optionally substituted C₁-C₆ alkyl;
R⁷ is —(CH₂)ₚ-Q-(CH₂)_{q}—, wherein p and q are independently 0, 1, or 2, and Q is a bond (absent), —O—, —S—, —S(O)—, —S(O)₂—, —NR¹², —CH(OH)—, —C(═O)—, —C(═O)O—, or —OC(═O)—,
   wherein 2≤(p+q)≤4 if Q is a bond,
   wherein 1≤(p+q)≤3 if Q is —O—, S—, —S(O)—, —S(O)₂—, —NR¹², —CH(OH)—, or —C(═O)—,
   wherein 0≤(p+q)≤2 if Q is —C(═O)O— or —OC(═O)—, and
   wherein each CH₂ in R⁷ is optionally substituted with at least one substituent selected from the group consisting of methyl, OR¹³, or halogen;
each occurrence of R⁸ is independently selected from the group consisting of H, optionally substituted C₁-C₆ alkyl, and optionally substituted C₃-C₈ cycloalkyl;
each occurrence of R^{9a}, R^{9b}, R^{9c}, R^{9d}, R^{9e}, R^{9f}, R^{9g}, and R^{9h} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, optionally substituted phenyl, optionally substituted heterocyclyl, optionally substituted heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R¹⁰ is independently selected from the group consisting of H, optionally substituted C₁-C₆ alkyl, optionally substituted C₃-C₈ cycloalkyl, optionally substituted phenyl, and optionally substituted hetereoaryl;
each occurrence of R¹¹ is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, optionally substituted phenyl, optionally substituted heterocyclyl, optionally substituted heteroaryl, —S(optionally substituted C₁-C₆ alkyl), —SO(optionally substituted C₁-C₆ alkyl), —SO₂(optionally substituted C₁-C₆ alkyl), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R¹² is independently selected from the group consisting of H, optionally substituted C₁-C₆ alkyl, optionally substituted C₁-C₆ hydroxyalkyl, optionally substituted C₃-C₈ cycloalkyl, optionally substituted phenyl, optionally substituted heteroaryl, and optionally substituted C₁-C₆ acyl;
each occurrence of R¹³ is independently selected from the group consisting of H, optionally substituted C₁-C₆ alkyl, and —C(═O)C₁-C₆ alkyl.

Embodiment 2 provides the compound of Embodiment 1, wherein each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —C(═O)OR^{b}, —OC(═O)R^{b}, —SR^{b}, —S(═O)R^{b}, —S(═O)₂R^{b}, N(R^{b})S(═O)₂R^{b}, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

Embodiment 3 provides the compound of any of Embodiments 1-2, wherein each occurrence of alkyl, alkenyl, alkynyl, or cycloalkyl is independently optionally substituted with at least one substituent selected from the group consisting of C₁-C₆ alkyl, C₃-C₈ cycloalkyl, halo, cyano (—CN), —OR^{a}, optionally substituted phenyl, optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

Embodiment 4 provides the compound of any of Embodiments 1-3, wherein R² is selected from the group consisting of

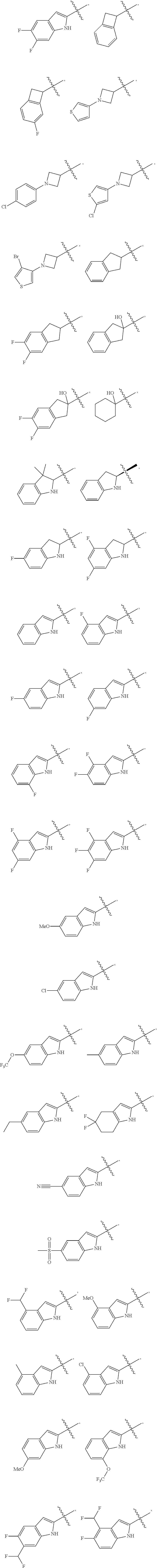

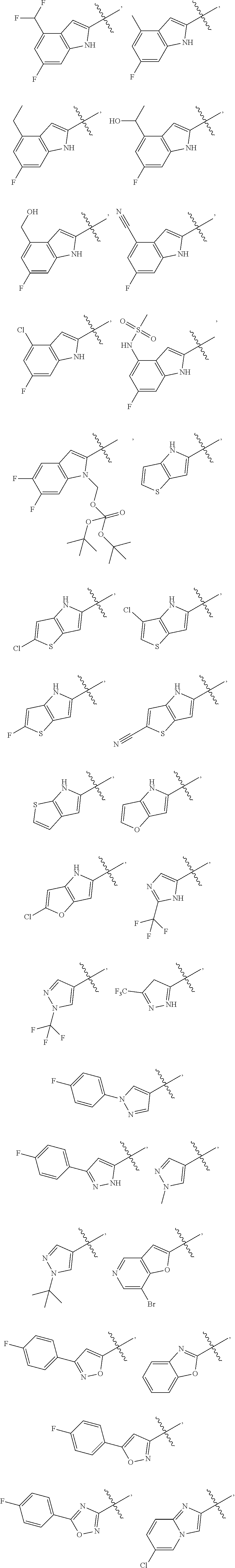

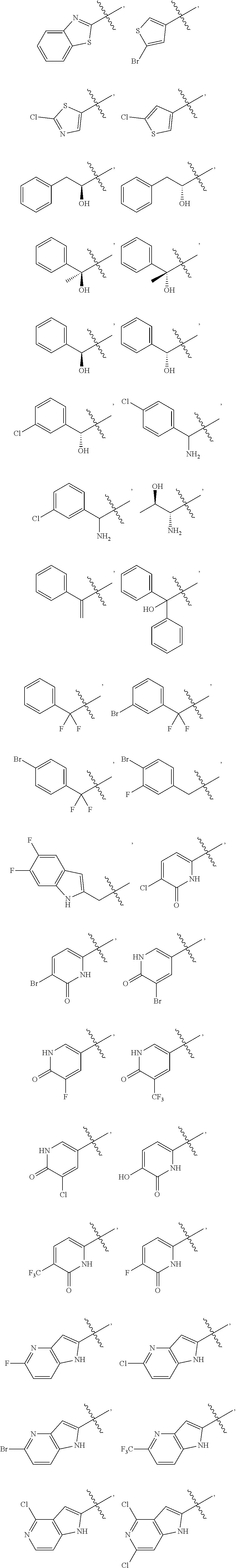

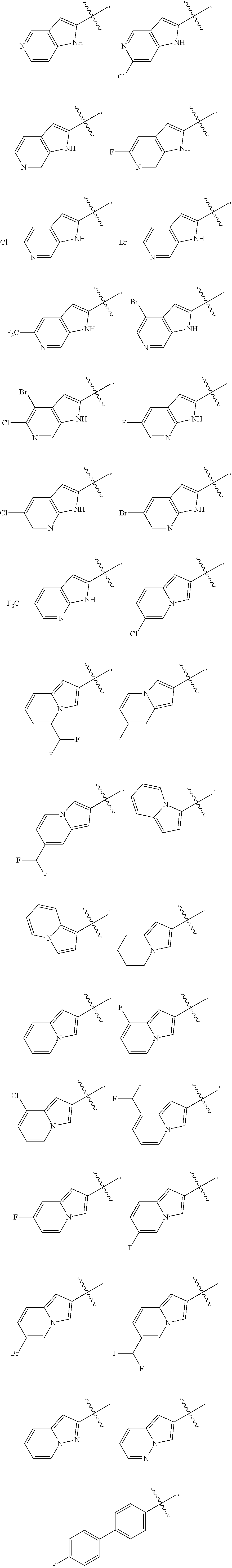

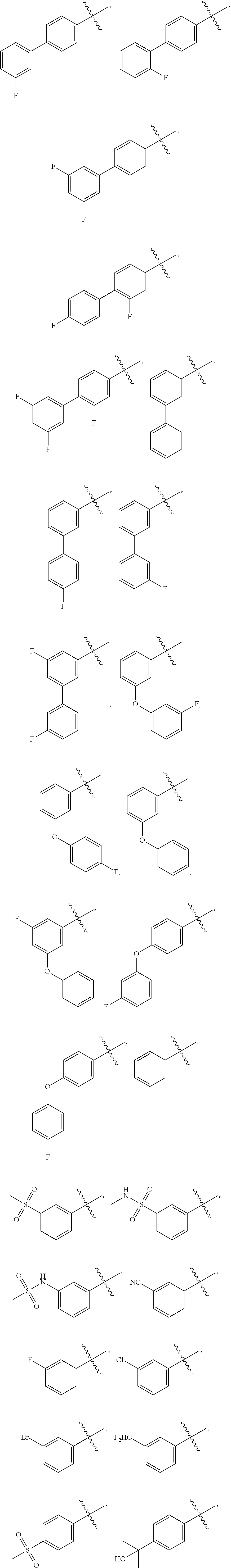

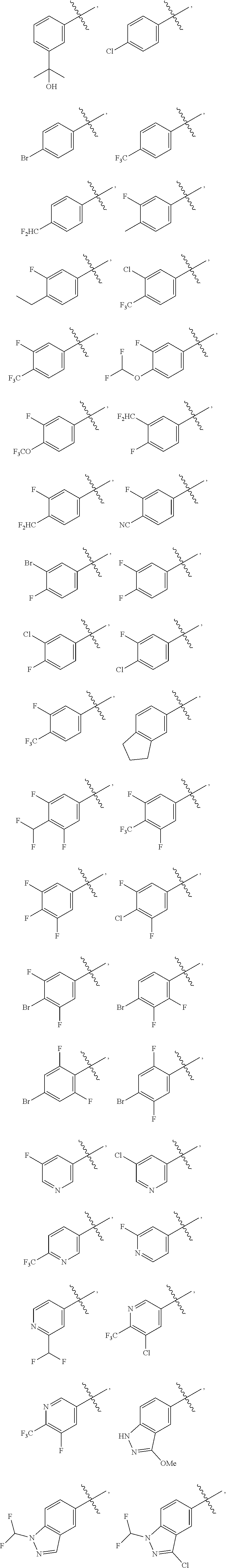

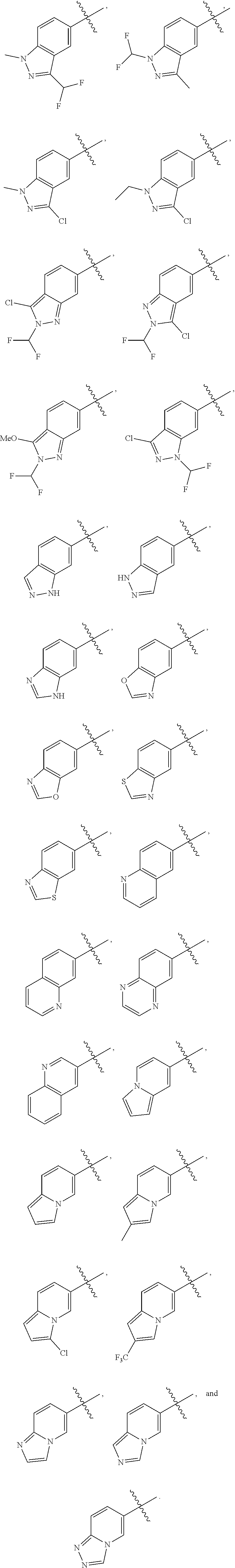

Embodiment 5 provides the compound of any of Embodiments 1-4, wherein each of R^{3a} and R^{3b} is independently H or methyl.

Embodiment 6 provides the compound of any of Embodiments 1-5, wherein R⁵ is selected from the group consisting of H, methyl, ethyl, isopropyl, n-propyl, cyclopropyl, n-butyl, isobutyl, sec-butyl, tert-butyl, cyclobutyl, isopropylmethyl, —(CH₂)₂₋₆OH, —(CH₂)₂₋₆O(C₁-C₆ alkyl), ¹³CD₃, optionally substituted benzyl, and optionally substituted phenyl.

Embodiment 7 provides the compound of any of Embodiments 1-6, wherein R⁶ is selected from the group consisting of H, D, and CH₃.

Embodiment 8 provides the compound of any of Embodiments 1-7, which is selected from the group consisting of:

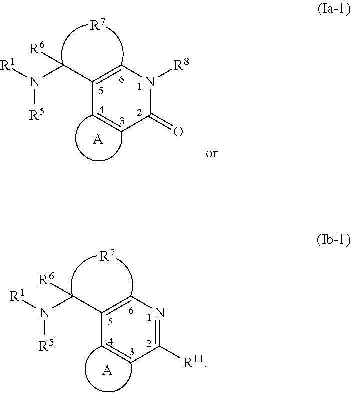

Embodiment 9 provides the compound of any of Embodiments 1-8, which is selected from the group consisting of:

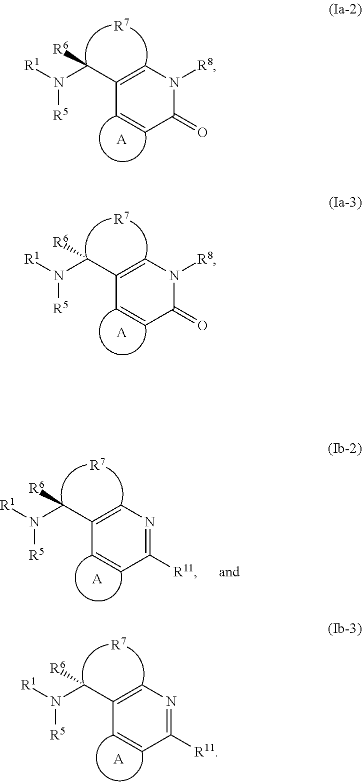

Embodiment 10 provides the compound of any of Embodiments 1-9, which is selected from the group consisting of:

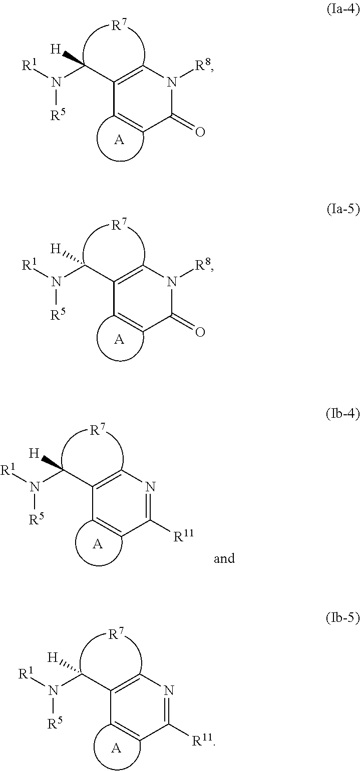

Embodiment 11 provides the compound of any of Embodiments 1-10, which is selected from the group consisting of:

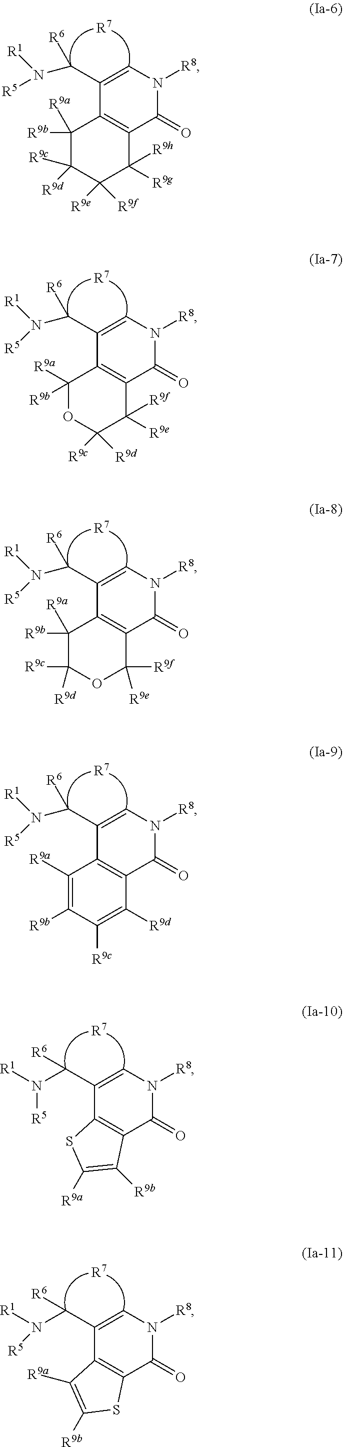

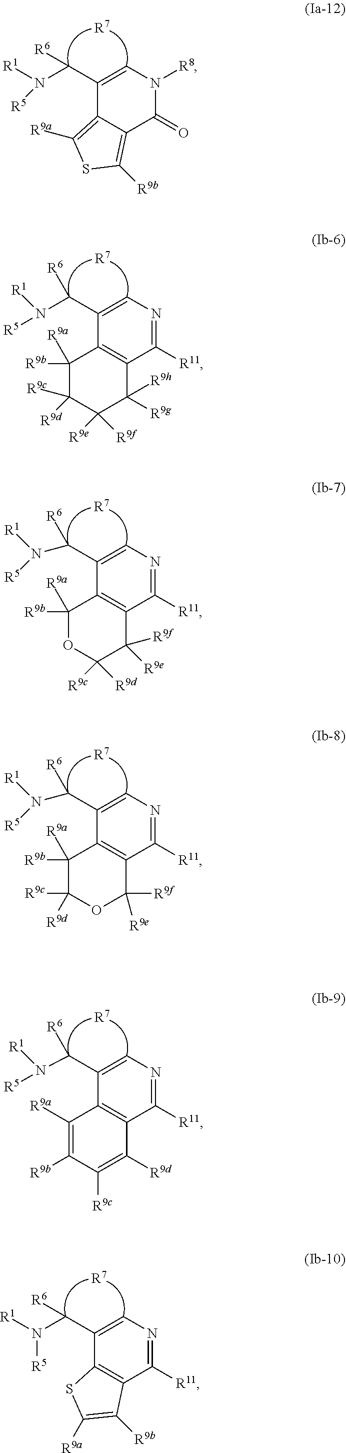

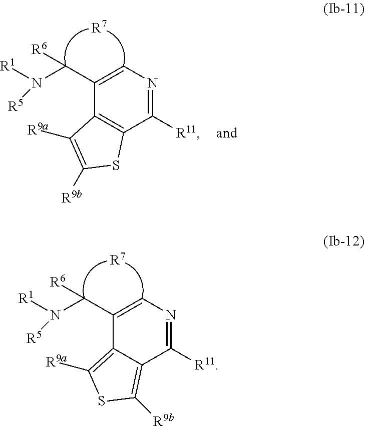

Embodiment 12 provides the compound of any of Embodiments 1-11, which is at least one selected from the group consisting of:
N-(8-fluoro-6-oxo-1,4,5,6-tetrahydro-2H-pyrano[3,4-c]isoquinolin-1-yl)-N-methyl-1H-indole-2-carboxamide;
N-(8-fluoro-6-oxo-1,4,5,6-tetrahydro-2H-pyrano[3,4-c]isoquinolin-1-yl)-N-methylindoline-2-carboxamide;
N-(8,9-difluoro-6-oxo-1,4,5,6-tetrahydro-2H-pyrano[3,4-c]isoquinolin-1-yl)-5-fluoro-N-methylindoline-2-carboxamide;
N-(8,9-difluoro-6-oxo-1,4,5,6-tetrahydro-2H-pyrano[3,4-c]isoquinolin-1-yl)-8-fluoro-N-methylindolizine-2-carboxamide;
N-(8,9-difluoro-6-oxo-1,4,5,6-tetrahydro-2H-pyrano[3,4-c]isoquinolin-1-yl)-N-methylindolizine-2-carboxamide;
N-(8,9-difluoro-6-oxo-1,4,5,6-tetrahydro-2H-pyrano[3,4-c]isoquinolin-1-yl)-4,6-difluoro-N-methylindoline-2-carboxamide;
8-chloro-N-(8,9-difluoro-6-oxo-1,4,5,6-tetrahydro-2H-pyrano[3,4-c]isoquinolin-1-yl)-N-methylindolizine-2-carboxamide;
4-fluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-4-fluoro-N-methyl-1H-indole-2-carboxamide;
N-(8,9-difluoro-6-oxo-1,4,5,6-tetrahydro-2H-pyrano[3,4-c]isoquinolin-1-yl)-6-fluoro-N-methyl-1H-indole-2-carboxamide;
N-(8,9-difluoro-6-oxo-1,4,5,6-tetrahydro-2H-pyrano[3,4-c]isoquinolin-1-yl)-5-fluoro-N-methyl-1H-indole-2-carboxamide;
5-fluoro-N-(8-fluoro-6-oxo-1,4,5,6-tetrahydro-2H-pyrano[3,4-c]isoquinolin-1-yl)-N-methyl-1H-indole-2-carboxamide;
6-fluoro-N-(8-fluoro-6-oxo-1,4,5,6-tetrahydro-2H-pyrano[3,4-c]isoquinolin-1-yl)-N-methyl-1H-indole-2-carboxamide;
7-fluoro-N-(8-fluoro-6-oxo-1,4,5,6-tetrahydro-2H-pyrano[3,4-c]isoquinolin-1-yl)-N-methyl-1H-indole-2-carboxamide;
4,6-difluoro-N-(8-fluoro-6-oxo-1,4,5,6-tetrahydro-2H-pyrano[3,4-c]isoquinolin-1-yl)-N-methyl-1H-indole-2-carboxamide;
4,5-difluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-7-fluoro-N-methyl-1H-indole-2-carboxamide;
N-(8,9-difluoro-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-4,6-difluoro-N-methyl-1H-indole-2-carboxamide;
N-(8,9-difluoro-6-oxo-1,4,5,6-tetrahydro-2H-pyrano[3,4-c]isoquinolin-1-yl)-4,5-difluoro-N-methyl-1H-indole-2-carboxamide;
N-(8,9-difluoro-6-oxo-1,4,5,6-tetrahydro-2H-pyrano[3,4-c]isoquinolin-1-yl)-5,5-difluoro-N-methyl-4,5,6,7-tetrahydro-1H-indole-2-carboxamide;
5,6-difluoro-N-(8-fluoro-6-oxo-1,4,5,6-tetrahydro-2H-pyrano[3,4-c]isoquinolin-1-yl)-N-methyl-1H-indole-2-carboxamide;
6-chloro-N-(8,9-difluoro-6-oxo-1,4,5,6-tetrahydro-2H-pyrano[3,4-c]isoquinolin-1-yl)-N-methylimidazo[1,2-a]pyridine-2-carboxamide;
6-chloro-N-(8-fluoro-6-oxo-1,4,5,6-tetrahydro-2H-pyrano[3,4-c]isoquinolin-1-yl)-N-methylimidazo[1,2-a]pyridine-2-carboxamide;
6-chloro-N-(8-fluoro-6-oxo-1,4,5,6-tetrahydro-2H-pyrano[3,4-c]isoquinolin-1-yl)-N-ethylimidazo[1,2-a]pyridine-2-carboxamide;
N-(8,9-difluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N,3,3-trimethylindoline-2-carboxamide;
N-(8-fluoro-3-methyl-6-oxo-1,2,3,4,5,6-hexahydrobenzo[c][1,7]naphthyridin-1-yl)-N-methyl-1H-indole-2-carboxamide;
N-(8,9-difluoro-3-methyl-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methylpyrazolo[1,5-a]pyridine-2-carboxamide;
N-(8,9-difluoro-6-oxo-1,4,5,6-tetrahydro-2H-pyrano[3,4-c]isoquinolin-1-yl)-N-methylpyrrolo[1,2-b]pyridazine-6-carboxamide;
N-(8,9-difluoro-6-oxo-1,4,5,6-tetrahydro-2H-pyrano[3,4-c]isoquinolin-1-yl)-N-methylquinoline-7-carboxamide;
N-(8,9-difluoro-6-oxo-1,4,5,6-tetrahydro-2H-pyrano[3,4-c]isoquinolin-1-yl)-N-methylquinoline-6-carboxamide;
N-(3-Acetyl-8-fluoro-6-oxo-1,2,3,4,5,6-hexahydrobenzo[c][1,7]naphthyridin-1-yl)-N-methyl-1H-indole-2-carboxamide;
N-(8,9-Difluoro-6-oxo-1,4,5,6-tetrahydro-2H-pyrano[3,4-c]isoquinolin-1-yl)-N-methyl-[1,2,4]triazolo[4,3-a]pyridine-6-carboxamide;
N-(8,9-Difluoro-6-oxo-1,4,5,6-tetrahydro-2H-pyrano[3,4-c]isoquinolin-1-yl)-N-methylquinoline-3-carboxamide;
N-(8,9-Difluoro-6-oxo-1,4,5,6-tetrahydro-2H-pyrano[3,4-c]isoquinolin-1-yl)-N-methylquinoxaline-6-carboxamide;
N-(8,9-Difluoro-6-oxo-1,4,5,6-tetrahydro-2H-pyrano[3,4-c]isoquinolin-1-yl)-N-methyl-6-(trifluoromethyl)nicotinamide;
N-(8,9-Difluoro-6-oxo-1,4,5,6-tetrahydro-2H-pyrano[3,4-c]isoquinolin-1-yl)-3-fluoro-N-methyl-4-(trifluoromethyl)benzamide;
4-Chloro-N-(8,9-difluoro-6-oxo-1,4,5,6-tetrahydro-2H-pyrano[3,4-c]isoquinolin-1-yl)-3-fluoro-N-methylbenzamide;
N-(8,9-Difluoro-6-oxo-1,4,5,6-tetrahydro-2H-pyrano[3,4-c]isoquinolin-1-yl)-3,4,5-trifluoro-N-methylbenzamide;
N-(8-Fluoro-6-oxo-1,2,3,4,5,6-hexahydrobenzo[c][1,7]naphthyridin-1-yl)-N-methyl-1H-indole-2-carboxamide;
N-(8-Fluoro-3-(2-hydroxyethyl)-6-oxo-1,2,3,4,5,6-hexahydrobenzo[c][1,7]naphthyridin-1-yl)-N-methyl-1H-indole-2-carboxamide;
N-(3-Acetyl-8,9-difluoro-6-oxo-1,2,3,4,5,6-hexahydrobenzo[c][1,7]naphthyridin-1-yl)-N-methyl-1H-indole-2-carboxamide;
N-(8,9-Difluoro-6-oxo-1,4,5,6-tetrahydro-2H-pyrano[3,4-c]isoquinolin-1-yl)-5-fluoro-N-methylnicotinamide;
5-Chloro-N-(8,9-difluoro-6-oxo-1,4,5,6-tetrahydro-2H-pyrano[3,4-c]isoquinolin-1-yl)-N-methylnicotinamide;
N-(8,9-Difluoro-6-oxo-1,4,5,6-tetrahydro-2H-pyrano[3,4-c]isoquinolin-1-yl)-2-fluoro-N-methylisonicotinamide;
N-(8,9-Difluoro-6-oxo-1,4,5,6-tetrahydro-2H-pyrano[3,4-c]isoquinolin-1-yl)-3-(difluoromethyl)-N-methylbenzamide;
N-(8,9-Difluoro-6-oxo-1,4,5,6-tetrahydro-2H-pyrano[3,4-c]isoquinolin-1-yl)-2-hydroxy-N-methyl-3-phenylpropanamide;
N-(8,9-Difluoro-3-(2-hydroxyethyl)-6-oxo-1,2,3,4,5,6-hexahydrobenzo[c][1,7]naphthyridin-1-yl)-N-methyl-1H-indole-2-carboxamide;
N-(8,9-Difluoro-6-oxo-1,4,5,6-tetrahydro-2H-pyrano[3,4-c]isoquinolin-1-yl)-N-methylbenzamide;
N-(8,9-Difluoro-6-oxo-1,4,5,6-tetrahydro-2H-pyrano[3,4-c]isoquinolin-1-yl)-3,4-difluoro-N-methylbenzamide;
N-(8,9-Difluoro-6-oxo-1,4,5,6-tetrahydro-2H-pyrano[3,4-c]isoquinolin-1-yl)-2-hydroxy-N-methyl-2-phenylpropanamide;
N-(8,9-Difluoro-6-oxo-1,4,5,6-tetrahydro-2H-pyrano[3,4-c]isoquinolin-1-yl)-2-hydroxy-N-methyl-2-phenylacetamide;
N-(8,9-Difluoro-6-oxo-1,4,5,6-tetrahydro-2H-pyrano[3,4-c]isoquinolin-1-yl)-4,5,6-trifluoro-N-methyl-1H-indole-2-carboxamide;
N-(8,9-Difluoro-6-oxo-1,4,5,6-tetrahydro-2H-pyrano[3,4-c]isoquinolin-1-yl)-N-methyl-4-(trifluoromethyl)benzamide;
4-Chloro-N-(8,9-difluoro-6-oxo-1,4,5,6-tetrahydro-2H-pyrano[3,4-c]isoquinolin-1-yl)-N-methylbenzamide;
4-Cyano-N-(8,9-difluoro-6-oxo-1,4,5,6-tetrahydro-2H-pyrano[3,4-c]isoquinolin-1-yl)-3-fluoro-N-methylbenzamide;
N-(8,9-Difluoro-6-oxo-1,2,3,4,5,6-hexahydrobenzo[c][1,7]naphthyridin-1-yl)-5-fluoro-N-methyl-1H-indole-2-carboxamide;
N-(8,9-Difluoro-6-oxo-1,4,5,6-tetrahydro-2H-thiopyrano[3,4-c]isoquinolin-1-yl)-8-fluoro-N-methylindolizine-2-carboxamide;
N-(8,9-Difluoro-6-oxo-1,2,3,4,5,6-hexahydrobenzo[c][1,7]naphthyridin-1-yl)-4-fluoro-N-methyl-1H-indole-2-carboxamide;
N-(8,9-Difluoro-6-oxo-1,2,3,4,5,6-hexahydrobenzo[c][1,7]naphthyridin-1-yl)-6-fluoro-N-methyl-1H-indole-2-carboxamide;
N-(8,9-Difluoro-6-oxo-1,4,5,6-tetrahydro-2H-pyrano[3,4-c]isoquinolin-1-yl)-3-(difluoromethyl)-4-fluoro-N-methylbenzamide;
N-(8,9-Difluoro-6-oxo-1,4,5,6-tetrahydro-2H-pyrano[3,4-c]isoquinolin-1-yl)-4-(difluoromethyl)-3-fluoro-N-methylbenzamide;
N-(8,9-Difluoro-6-oxo-1,4,5,6-tetrahydro-2H-pyrano[3,4-c]isoquinolin-1-yl)-4-(difluoromethyl)-N-methylbenzamide;
3-Chloro-N-(8,9-difluoro-6-oxo-1,4,5,6-tetrahydro-2H-pyrano[3,4-c]isoquinolin-1-yl)-4-fluoro-N-methylbenzamide;
N-(8,9-Difluoro-6-oxo-1,2,3,4,5,6-hexahydrobenzo[c][1,7]naphthyridin-1-yl)-7-fluoro-N-methyl-1H-indole-2-carboxamide;
N-(8,9-Difluoro-6-oxo-1,2,3,4,5,6-hexahydrobenzo[c][1,7]naphthyridin-1-yl)-5,6-difluoro-N-methyl-1H-indole-2-carboxamide;
N-(8,9-Difluoro-6-oxo-1,2,3,4,5,6-hexahydrobenzo[c][1,7]naphthyridin-1-yl)-4,6-difluoro-N-methyl-1H-indole-2-carboxamide;
N-(8,9-Difluoro-6-oxo-1,2,3,4,5,6-hexahydrobenzo[c][1,7]naphthyridin-1-yl)-4,5-difluoro-N-methyl-1H-indole-2-carboxamide;
6-Chloro-N-(8,9-difluoro-6-oxo-1,2,3,4,5,6-hexahydrobenzo[c][1,7]naphthyridin-1-yl)-N-methylindolizine-2-carboxamide;
N-(8,9-Difluoro-6-oxo-1,4,5,6-tetrahydro-2H-pyrano[3,4-c]isoquinolin-1-yl)-N-methylbenzo[d]thiazole-2-carboxamide;
N-(8,9-Difluoro-6-oxo-1,4,5,6-tetrahydro-2H-pyrano[3,4-c]isoquinolin-1-yl)-N-methylbenzo[d]oxazole-2-carboxamide;
N-(8,9-Difluoro-6-oxo-1,4,5,6-tetrahydro-2H-pyrano[3,4-c]isoquinolin-1-yl)-N-methyl-3-(trifluoromethyl)-1H-pyrazole-5-carboxamide;
N-(8,9-Difluoro-6-oxo-1,4,5,6-tetrahydro-2H-pyrano[3,4-c]isoquinolin-1-yl)-3-(4-fluorophenyl)-N-methyl-1H-pyrazole-5-carboxamide;
N-(8,9-Difluoro-6-oxo-1,4,5,6-tetrahydro-2H-pyrano[3,4-c]isoquinolin-1-yl)-3-fluoro-N-methylbenzamide;
3-Chloro-N-(8,9-difluoro-6-oxo-1,4,5,6-tetrahydro-2H-pyrano[3,4-c]isoquinolin-1-yl)-N-methylbenzamide;
3-Bromo-N-(8,9-difluoro-6-oxo-1,4,5,6-tetrahydro-2H-pyrano[3,4-c]isoquinolin-1-yl)-N-methylbenzamide;
3-Bromo-N-(8,9-difluoro-6-oxo-1,4,5,6-tetrahydro-2H-pyrano[3,4-c]isoquinolin-1-yl)-4-fluoro-N-methylbenzamide;
4-Bromo-N-(8,9-difluoro-6-oxo-1,4,5,6-tetrahydro-2H-pyrano[3,4-c]isoquinolin-1-yl)-N-methylbenzamide;
3-Fluoro-N-methyl-N-(6-oxo-1,4,5,6-tetrahydro-2H-pyrano[3,4-c]isoquinolin-1-yl)-4-(trifluoromethyl)benzamide;
3-Fluoro-N-methyl-N-(4-oxo-4,5,8,9-tetrahydro-6H-pyrano[3,4-b]thieno[3,4-d]pyridin-9-yl)-4-(trifluoromethyl)benzamide;
N-(8-Cyano-6-oxo-1,4,5,6-tetrahydro-2H-pyrano[3,4-c]isoquinolin-1-yl)-3-fluoro-N-methyl-4-(trifluoromethyl)benzamide;
3-Cyano-N-(8,9-difluoro-6-oxo-1,4,5,6-tetrahydro-2H-pyrano[3,4-c]isoquinolin-1-yl)-N-methylbenzamide;
N-(8,9-Difluoro-6-oxo-1,4,5,6-tetrahydro-2H-pyrano[3,4-c]isoquinolin-1-yl)-3-fluoro-N-methyl-4-(trifluoromethoxy)benzamide;
N-(8,9-Difluoro-6-oxo-1,4,5,6-tetrahydro-2H-pyrano[3,4-c]isoquinolin-1-yl)-3-fluoro-N,4-dimethylbenzamide;
2-(3-Chlorophenyl)-N-(8,9-difluoro-6-oxo-1,4,5,6-tetrahydro-2H-pyrano[3,4-c]isoquinolin-1-yl)-2-hydroxy-N-methylacetamide;
N-(8,9-Difluoro-6-oxo-1,4,5,6-tetrahydro-2H-pyrano[3,4-c]isoquinolin-1-yl)-3,5-difluoro-N-methyl-4-(trifluoromethyl)benzamide;
5-Chloro-N-(8,9-difluoro-6-oxo-1,4,5,6-tetrahydro-2H-pyrano[3,4-c]isoquinolin-1-yl)-N-methylthiophene-3-carboxamide;
3-Chloro-N-(8,9-difluoro-6-oxo-1,4,5,6-tetrahydro-2H-pyrano[3,4-c]isoquinolin-1-yl)-4-fluoro-N-methylbenzenesulfonamide;
3-Chloro-N-(8,9-difluoro-6-oxo-1,4,5,6-tetrahydro-2H-pyrano[3,4-c]isoquinolin-1-yl)-N-methylbenzenesulfonamide;
3-Cyano-N-(8,9-difluoro-6-oxo-1,4,5,6-tetrahydro-2H-pyrano[3,4-c]isoquinolin-1-yl)-N-methylbenzenesulfonamide;
3-Cyano-N-(8,9-difluoro-6-oxo-1,4,5,6-tetrahydro-2H-pyrano[3,4-c]isoquinolin-1-yl)-4-fluoro-N-methylbenzenesulfonamide;
N-(8,9-Difluoro-6-oxo-1,4,5,6-tetrahydro-2H-pyrano[3,4-c]isoquinolin-1-yl)-4-ethyl-3-fluoro-N-methylbenzamide;
N-(8,9-difluoro-6-oxo-1,4,5,6-tetrahydro-2H-pyrano[3,4-c]isoquinolin-1-yl)indolizine-2-carboxamide;
N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
N-(8,9-difluoro-3,3-dioxido-6-oxo-1,4,5,6-tetrahydro-2H-thiopyrano[3,4-c]isoquinolin-1-yl)-8-fluoro-N-methylindolizine-2-carboxamide;
N-(8,9-difluoro-6-oxo-1,4,5,6-tetrahydro-2H-pyrano[3,4-c]isoquinolin-1-yl)-4-(difluoromethoxy)-3-fluoro-N-methylbenzamide;
N-(8,9-difluoro-6-oxo-1,4,5,6-tetrahydro-2H-pyrano[3,4-c]isoquinolin-1-yl)-4-(2-hydroxypropan-2-yl)-N-methylbenzamide;
N-(8,9-difluoro-6-oxo-1,4,5,6-tetrahydro-2H-pyrano[3,4-c]isoquinolin-1-yl)-3-(2-hydroxypropan-2-yl)-N-methylbenzamide;
N-(8,9-difluoro-6-oxo-1,4,5,6-tetrahydro-2H-pyrano[3,4-c]isoquinolin-1-yl)-5,6-difluoro-1H-indole-2-carboxamide;
N-(8,9-difluoro-6-oxo-1,4,5,6-tetrahydro-2H-pyrano[3,4-c]isoquinolin-1-yl)-4,6-difluoro-1H-indole-2-carboxamide;
N-(8,9-difluoro-6-oxo-1,4,5,6-tetrahydro-2H-pyrano[3,4-c]isoquinolin-1-yl)-3-fluoro-4-(trifluoromethyl)benzamide;
4-chloro-N-(8,9-difluoro-6-oxo-1,4,5,6-tetrahydro-2H-pyrano[3,4-c]isoquinolin-1-yl)-3-fluorobenzamide;
N-(8,9-difluoro-6-oxo-1,2,3,4,5,6-hexahydrobenzo[c][1,7]naphthyridin-1-yl)-8-fluoro-N-methylindolizine-2-carboxamide;
N-(8,9-difluoro-6-oxo-1,2,3,4,5,6-hexahydrobenzo[c][1,7]naphthyridin-1-yl)-7-fluoro-N-methylindolizine-2-carboxamide;
4-bromo-N-(8,9-difluoro-6-oxo-1,2,3,4,5,6-hexahydrobenzo[c][1,7]naphthyridin-1-yl)-3,5-difluoro-N-methylbenzamide;
4-bromo-3,5-difluoro-N-(8-fluoro-6-oxo-1,4,5,6-tetrahydro-2H-pyrano[3,4-c]isoquinolin-1-yl)-N-methylbenzamide;
4-chloro-N-(8,9-difluoro-6-oxo-1,2,3,4,5,6-hexahydrobenzo[c][1,7]naphthyridin-1-yl)-3,5-difluoro-N-methylbenzamide;
4-bromo-3,5-difluoro-N-(8-fluoro-6-oxo-1,2,3,4,5,6-hexahydrobenzo[c][1,7]naphthyridin-1-yl)-N-methylbenzamide;
4-bromo-N-(8,9-difluoro-6-oxo-1,4,5,6-tetrahydro-2H-pyrano[3,4-c]isoquinolin-1-yl)-3,5-difluoro-N-methylbenzamide;
4-chloro-N-(8,9-difluoro-6-oxo-1,4,5,6-tetrahydro-2H-pyrano[3,4-c]isoquinolin-1-yl)-3,5-difluoro-N-methylbenzamide;
4-chloro-3,5-difluoro-N-(8-fluoro-6-oxo-1,4,5,6-tetrahydro-2H-pyrano[3,4-c]isoquinolin-1-yl)-N-methylbenzamide;
4-chloro-3,5-difluoro-N-(8-fluoro-6-oxo-1,2,3,4,5,6-hexahydrobenzo[c][1,7]naphthyridin-1-yl)-N-methylbenzamide;
4-(difluoromethyl)-3,5-difluoro-N-(8-fluoro-6-oxo-1,4,5,6-tetrahydro-2H-pyrano[3,4-c]isoquinolin-1-yl)-N-methylbenzamide;
N-(8,9-difluoro-6-oxo-1,4,5,6-tetrahydro-2H-pyrano[3,4-c]isoquinolin-1-yl)-4-(difluoromethyl)-3,5-difluoro-N-methylbenzamide;
4-(difluoromethyl)-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,2,3,4,5,6-hexahydrobenzo[c][1,7]naphthyridin-1-yl)-4-(difluoromethyl)-3,5-difluoro-N-methylbenzamide;
4-(difluoromethyl)-3,5-difluoro-N-(8-fluoro-6-oxo-1,2,3,4,5,6-hexahydrobenzo[c][1,7]naphthyridin-1-yl)-N-methylbenzamide;
N-(8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-N,1-dimethyl-1H-pyrazole-4-carboxamide;
N-(8,9-difluoro-6-oxo-1,4,5,6-tetrahydro-2H-pyrano[3,4-c]isoquinolin-1-yl)-N-methyl-2-phenylacrylamide;
N-(8,9-difluoro-6-oxo-1,4,5,6-tetrahydro-2H-pyrano[3,4-c]isoquinolin-1-yl)-5-fluoro-N-methyl-1H-pyrrolo[2,3-b]pyridine-2-carboxamide;
5-chloro-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
5-bromo-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-3-carboxamide;
5-bromo-N-(8,9-difluoro-6-oxo-1,4,5,6-tetrahydro-2H-pyrano[3,4-c]isoquinolin-1-yl)-N-methylthiophene-3-carboxamide;
N-(8,9-difluoro-6-oxo-1,4,5,6-tetrahydro-2H-pyrano[3,4-c]isoquinolin-1-yl)-N-methyl-2,3-dihydro-1H-indene-2-carboxamide;
1-(tert-butyl)-N-(8,9-difluoro-6-oxo-1,4,5,6-tetrahydro-2H-pyrano[3,4-c]isoquinolin-1-yl)-N-methyl-1H-pyrazole-4-carboxamide;
N-(8,9-difluoro-6-oxo-1,4,5,6-tetrahydro-2H-pyrano[3,4-c]isoquinolin-1-yl)-N-methyl-1-(trifluoromethyl)-1H-pyrazole-4-carboxamide;
4-bromo-N-(8,9-difluoro-6-oxo-1,4,5,6-tetrahydro-2H-pyrano[3,4-c]isoquinolin-1-yl)-N-methyl-1H-pyrrole-2-carboxamide;
2-amino-2-(4-chlorophenyl)-N-(8,9-difluoro-6-oxo-1,4,5,6-tetrahydro-2H-pyrano[3,4-c]isoquinolin-1-yl)-N-methylacetamide;
5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
2-amino-2-(3-chlorophenyl)-N-(8,9-difluoro-6-oxo-1,4,5,6-tetrahydro-2H-pyrano[3,4-c]isoquinolin-1-yl)-N-methylacetamide;
5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
5-chloro-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
N-(8,9-difluoro-6-oxo-1,2,3,4,5,6-hexahydrobenzo[c][1,7]naphthyridin-1-yl)-4-(difluoromethyl)-N-methyl-1H-indole-2-carboxamide;
4-(difluoromethyl)-N-(8-fluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-1-hydroxy-N-methylcyclohexane-1-carboxamide;
N-(8,9-difluoro-6-oxo-1,4,5,6-tetrahydro-2H-pyrano[3,4-c]isoquinolin-1-yl)-6-(difluoromethyl)-5-fluoro-N-methyl-1H-indole-2-carboxamide;
5-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-b]pyridine-2-carboxamide;
5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
6-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methyl-5-(trifluoromethyl)-1H-pyrrolo[3,2-b]pyridine-2-carboxamide;
N-(8,9-difluoro-6-oxo-1,2,3,4,5,6-hexahydrobenzo[c][1,7]naphthyridin-1-yl)-6-(difluoromethyl)-5-fluoro-N-methyl-1H-indole-2-carboxamide;
N-(8,9-difluoro-6-oxo-1,2,3,4,5,6-hexahydrobenzo[c][1,7]naphthyridin-1-yl)-6-fluoro-N-methylindolizine-2-carboxamide;
N-(8,9-difluoro-6-oxo-1,4,5,6-tetrahydro-2H-pyrano[3,4-c]isoquinolin-1-yl)-6-fluoro-N-methylindolizine-2-carboxamide;
N-(8,9-difluoro-6-oxo-1,4,5,6-tetrahydro-2H-pyrano[3,4-c]isoquinolin-1-yl)-N-methyl-2-(trifluoromethyl)-1H-imidazole-5-carboxamide;
N-(8,9-difluoro-6-oxo-1,4,5,6-tetrahydro-2H-pyrano[3,4-c]isoquinolin-1-yl)-5-fluoro-N-methyl-6-oxo-1,6-dihydropyridine-3-carboxamide;
N-(8,9-difluoro-6-oxo-1,4,5,6-tetrahydro-2H-pyrano[3,4-c]isoquinolin-1-yl)-N-methylindolizine-6-carboxamide;
N-(8,9-difluoro-6-oxo-1,4,5,6-tetrahydro-2H-pyrano[3,4-c]isoquinolin-1-yl)-N-methylindolizine-7-carboxamide;
N-(8,9-difluoro-6-oxo-1,4,5,6-tetrahydro-2H-pyrano[3,4-c]isoquinolin-1-yl)-2-hydroxy-N-methyl-2,2-diphenylacetamide;
N-(8,9-difluoro-6-oxo-1,4,5,6-tetrahydro-2H-pyrano[3,4-c]isoquinolin-1-yl)-4-(difluoromethyl)-6-fluoro-N-methyl-1H-indole-2-carboxamide;
N-(8,9-difluoro-6-oxo-1,4,5,6-tetrahydro-2H-pyrano[3,4-c]isoquinolin-1-yl)-7-fluoro-N-methylindolizine-2-carboxamide;
N-(8,9-difluoro-6-oxo-1,4,5,6-tetrahydro-2H-pyrano[3,4-c]isoquinolin-1-yl)-5-fluoro-N-methyl-1H-pyrrolo[3,2-b]pyridine-2-carboxamide;
N-(8,9-difluoro-6-oxo-1,4,5,6-tetrahydro-2H-pyrano[3,4-c]isoquinolin-1-yl)-5,6-difluoro-N-methyl-2,3-dihydro-1H-indene-2-carboxamide;
N-(8,9-difluoro-6-oxo-1,4,5,6-tetrahydro-2H-pyrano[3,4-c]isoquinolin-1-yl)-1-(4-fluorophenyl)-N-methyl-1H-pyrazole-4-carboxamide;
N-(8,9-difluoro-6-oxo-1,4,5,6-tetrahydro-2H-pyrano[3,4-c]isoquinolin-1-yl)-3-(4-fluorophenyl)-N-methylisoxazole-5-carboxamide;
N-(8,9-difluoro-6-oxo-1,4,5,6-tetrahydro-2H-pyrano[3,4-c]isoquinolin-1-yl)-4-(3-fluorophenoxy)-N-methylbenzamide;
4-(difluoromethyl)-6-fluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-5-methyl-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-6-methoxy-1,4-dihydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-N-methyl-5,6,7,8-tetrahydroindolizine-2-carboxamide;
4-bromo-N-(8,9-difluoro-6-oxo-1,4,5,6-tetrahydro-2H-pyrano[3,4-c]isoquinolin-1-yl)-2,3-difluoro-N-methylbenzamide;
4-bromo-N-(8,9-difluoro-6-oxo-1,4,5,6-tetrahydro-2H-pyrano[3,4-c]isoquinolin-1-yl)-2,5-difluoro-N-methylbenzamide;
4-bromo-N-(8,9-difluoro-6-oxo-1,4,5,6-tetrahydro-2H-pyrano[3,4-c]isoquinolin-1-yl)-2,6-difluoro-N-methylbenzamide;
N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-2-carboxamide;
N-(8,9-difluoro-6-oxo-1,4,5,6-tetrahydro-2H-pyrano[3,4-c]isoquinolin-1-yl)-N-methylindolizine-3-carboxamide;
N-(8,9-difluoro-6-oxo-1,4,5,6-tetrahydro-2H-pyrano[3,4-c]isoquinolin-1-yl)-4-(4-fluorophenoxy)-N-methylbenzamide;
N-(8,9-difluoro-6-oxo-1,4,5,6-tetrahydro-2H-pyrano[3,4-c]isoquinolin-1-yl)-4′-fluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-c]pyridine-2-carboxamide;
2-(3-bromophenyl)-N-(8,9-difluoro-6-oxo-1,4,5,6-tetrahydro-2H-pyrano[3,4-c]isoquinolin-1-yl)-2,2-difluoro-N-methylacetamide;
2-(4-bromophenyl)-N-(8,9-difluoro-6-oxo-1,4,5,6-tetrahydro-2H-pyrano[3,4-c]isoquinolin-1-yl)-2,2-difluoro-N-methylacetamide;
N-(8,9-difluoro-6-oxo-1,4,5,6-tetrahydro-2H-pyrano[3,4-c]isoquinolin-1-yl)-4-(difluoromethyl)-5-fluoro-N-methyl-1H-indole-2-carboxamide;
4-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,2,3,4,5,6-hexahydrobenzo[c][1,7]naphthyridin-1-yl)-4-(difluoromethyl)-6-fluoro-N-methyl-1H-indole-2-carboxamide;
4-(difluoromethyl)-6-fluoro-N-(8-fluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methylindolizine-1-carboxamide;
N-(8,9-difluoro-6-oxo-1,4,5,6-tetrahydro-2H-pyrano[3,4-c]isoquinolin-1-yl)-2,4′-difluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-5-(4-fluorophenyl)-N-methylisoxazole-3-carboxamide;
N-(8,9-difluoro-6-oxo-1,2,3,4,5,6-hexahydrobenzo[c][1,7]naphthyridin-1-yl)-4-(difluoromethyl)-5-fluoro-N-methyl-1H-indole-2-carboxamide;
N-(8,9-difluoro-6-oxo-1,4,5,6-tetrahydro-2H-pyrano[3,4-c]isoquinolin-1-yl)-5-(difluoromethyl)-N-methylindolizine-2-carboxamide;
5-bromo-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
N-(8,9-difluoro-6-oxo-1,4,5,6-tetrahydro-2H-pyrano[3,4-c]isoquinolin-1-yl)-N-methylbicyclo[4.2.0]octa-1(6),2,4-triene-7-carboxamide;
4-(difluoromethyl)-5-fluoro-N-(8-fluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-2-hydroxy-N-methyl-2,3-dihydro-1H-indene-2-carboxamide;
N-(8,9-difluoro-6-oxo-1,4,5,6-tetrahydro-2H-pyrano[3,4-c]isoquinolin-1-yl)-5,6-difluoro-2-hydroxy-N-methyl-2,3-dihydro-1H-indene-2-carboxamide;
N-(8,9-difluoro-6-oxo-1,4,5,6-tetrahydro-2H-pyrano[3,4-c]isoquinolin-1-yl)-N,2-dimethylindolizine-6-carboxamide;
N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
N-(8,9-difluoro-6-oxo-1,4,5,6-tetrahydro-2H-pyrano[3,4-c]isoquinolin-1-yl)-4-fluoro-N-methylbicyclo[4.2.0]octa-1(6),2,4-triene-7-carboxamide;
N-(6-(2-aminoethoxy)-8,9-difluoro-1,4-dihydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-N-methyl-1-(thiophen-3-yl)azetidine-3-carboxamide;
N-(8,9-difluoro-6-oxo-1,4,5,6-tetrahydro-2H-pyrano[3,4-c]isoquinolin-1-yl)-3′-fluoro-N-methyl-[1,1′-biphenyl]-4-carboxamide;
1-(4-bromothiophen-3-yl)-N-(8,9-difluoro-6-oxo-1,4,5,6-tetrahydro-2H-pyrano[3,4-c]isoquinolin-1-yl)-N-methylazetidine-3-carboxamide;
N-(8,9-difluoro-6-oxo-1,4,5,6-tetrahydro-2H-pyrano[3,4-c]isoquinolin-1-yl)-2′-fluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-3′,5′-difluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-2,3′,5′-trifluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-N-methyl-[1,1′-biphenyl]-3-carboxamide;
N-(8,9-difluoro-6-oxo-1,4,5,6-tetrahydro-2H-pyrano[3,4-c]isoquinolin-1-yl)-4′-fluoro-N-methyl-[1,1′-biphenyl]-3-carboxamide;
N-(8,9-difluoro-6-oxo-1,4,5,6-tetrahydro-2H-pyrano[3,4-c]isoquinolin-1-yl)-3′-fluoro-N-methyl-[1,1′-biphenyl]-3-carboxamide;
N-(8,9-difluoro-6-oxo-1,4,5,6-tetrahydro-2H-pyrano[3,4-c]isoquinolin-1-yl)-3′,5-difluoro-N-methyl-[1,1′-biphenyl]-3-carboxamide;
N-(8,9-difluoro-6-oxo-1,4,5,6-tetrahydro-2H-pyrano[3,4-c]isoquinolin-1-yl)-N-methyl-3-phenoxybenzamide;
N-(8,9-difluoro-6-oxo-1,4,5,6-tetrahydro-2H-pyrano[3,4-c]isoquinolin-1-yl)-3-(4-fluorophenoxy)-N-methylbenzamide;
N-(8,9-difluoro-6-oxo-1,4,5,6-tetrahydro-2H-pyrano[3,4-c]isoquinolin-1-yl)-N-methyl-6H-thieno[2,3-b]pyrrole-5-carboxamide;
N-(8,9-difluoro-6-oxo-1,4,5,6-tetrahydro-2H-pyrano[3,4-c]isoquinolin-1-yl)-N-methyl-4-(methylsulfonyl)benzamide;
N-(8,9-difluoro-6-oxo-1,4,5,6-tetrahydro-2H-pyrano[3,4-c]isoquinolin-1-yl)-N-methyl-3-(N-methylsulfamoyl)benzamide;
N-(8,9-difluoro-6-oxo-1,4,5,6-tetrahydro-2H-pyrano[3,4-c]isoquinolin-1-yl)-N-methyl-3-(methylsulfonamido)benzamide;
N-(8,9-difluoro-6-oxo-1,4,5,6-tetrahydro-2H-pyrano[3,4-c]isoquinolin-1-yl)-N-methyl-3-(methylsulfonyl)benzamide;
N-(8,9-difluoro-6-oxo-1,4,5,6-tetrahydro-2H-pyrano[3,4-c]isoquinolin-1-yl)-N-methylimidazo[1,5-a]pyridine-6-carboxamide;
1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
N-(8,9-difluoro-6-oxo-1,2,3,4,5,6-hexahydrobenzo[c][1,7]naphthyridin-1-yl)-3′-fluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-3-fluoro-N-methyl-5-phenoxybenzamide;
N-(8,9-difluoro-6-oxo-1,2,3,4,5,6-hexahydrobenzo[c][1,7]naphthyridin-1-yl)-3′-fluoro-N-methyl-[1,1′-biphenyl]-3-carboxamide;
N-(8,9-difluoro-6-oxo-1,4,5,6-tetrahydro-2H-pyrano[3,4-c]isoquinolin-1-yl)-N-methylimidazo[1,2-a]pyridine-6-carboxamide;
N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
6-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
N-(8,9-difluoro-6-oxo-1,4,5,6-tetrahydro-2H-pyrano[3,4-c]isoquinolin-1-yl)-5-fluoro-N-methyl-1H-pyrrolo[2,3-c]pyridine-2-carboxamide;
N-(8,9-difluoro-6-oxo-1,4,5,6-tetrahydro-2H-pyrano[3,4-c]isoquinolin-1-yl)-N-methyl-2-(trifluoromethyl)indolizine-6-carboxamide;
4-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
N-(8,9-difluoro-6-oxo-1,4,5,6-tetrahydro-2H-pyrano[3,4-c]isoquinolin-1-yl)-2-(difluoromethyl)-N-methylisonicotinamide;
N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
N-(8,9-difluoro-5-(2-hydroxyethyl)-6-oxo-1,4,5,6-tetrahydro-2H-pyrano[3,4-c]isoquinolin-1-yl)-5,6-difluoro-N-methyl-1H-indole-2-carboxamide;
N-(5-(2-aminoethyl)-8,9-difluoro-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-6-(2-hydroxyethoxy)-1,4-dihydro-2H-pyrano[3,4-c]isoquinolin-1-yl)-5,6-difluoro-N-methyl-1H-indole-2-carboxamide;
N-(8,9-difluoro-6-oxo-1,2,3,4,5,6-hexahydrobenzo[c][1,7]naphthyridin-1-yl)-N-methyl-3-(N-methylsulfamoyl)benzamide;
N-(8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-4,6-dioxo-1,4,5,6-tetrahydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-3-(3-fluorophenoxy)-N-methylbenzamide;
N-(8,9-difluoro-6-oxo-1,4,5,6-tetrahydro-2H-pyrano[3,4-c]isoquinolin-1-yl)-5-methoxy-N-methyl-1H-indole-2-carboxamide;
5-chloro-N-(8,9-difluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N,5-dimethyl-1H-indole-2-carboxamide;
N-(8,9-difluoro-6-oxo-1,4,5,6-tetrahydro-2H-pyrano[3,4-c]isoquinolin-1-yl)-N-methyl-5-(trifluoromethoxy)-1H-indole-2-carboxamide;
N-(8,9-difluoro-6-oxo-1,4,5,6-tetrahydro-2H-pyrano[3,4-c]isoquinolin-1-yl)-5-ethyl-N-methyl-1H-indole-2-carboxamide;
N-(8,9-difluoro-6-oxo-1,4,5,6-tetrahydro-2H-pyrano[3,4-c]isoquinolin-1-yl)-4-methoxy-N-methyl-1H-indole-2-carboxamide;
N-(8,9-difluoro-6-oxo-1,4,5,6-tetrahydro-2H-pyrano[3,4-c]isoquinolin-1-yl)-N,4-dimethyl-1H-indole-2-carboxamide;
1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
2-chloro-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
N-(8,9-difluoro-6-(methylamino)-1,4-dihydro-2H-pyrano[3,4-c]isoquinolin-1-yl)-5,6-difluoro-N-methyl-1H-indole-2-carboxamide;
N-(8,9-difluoro-6-((2-hydroxyethyl)amino)-1,4-dihydro-2H-pyrano[3,4-c]isoquinolin-1-yl)-5,6-difluoro-N-methyl-1H-indole-2-carboxamide;
N-(8,9-difluoro-6-oxo-1,2,3,4,5,6-hexahydrobenzo[c][1,7]naphthyridin-1-yl)-3-(3-fluorophenoxy)-N-methylbenzamide;
4-cyano-N-(8,9-difluoro-6-oxo-1,4,5,6-tetrahydro-2H-pyrano[3,4-c]isoquinolin-1-yl)-6-fluoro-N-methyl-1H-indole-2-carboxamide;
N-(8,9-difluoro-6-oxo-1,4,5,6-tetrahydro-2H-pyrano[3,4-c]isoquinolin-1-yl)-4-ethyl-6-fluoro-N-methyl-1H-indole-2-carboxamide;
4-chloro-N-(8,9-difluoro-6-oxo-1,4,5,6-tetrahydro-2H-pyrano[3,4-c]isoquinolin-1-yl)-6-fluoro-N-methyl-1H-indole-2-carboxamide;
N-(8,9-difluoro-6-oxo-1,4,5,6-tetrahydro-2H-pyrano[3,4-c]isoquinolin-1-yl)-6-fluoro-N,4-dimethyl-1H-indole-2-carboxamide;
5-cyano-N-(8,9-difluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methyl-5-(methylsulfonyl)-1H-indole-2-carboxamide;
4-chloro-N-(8,9-difluoro-6-oxo-1,4,5,6-tetrahydro-2H-pyrano[3,4-c]isoquinolin-1-yl)-N-methyl-1H-indole-2-carboxamide;
6-bromo-N-(8,9-difluoro-6-oxo-1,4,5,6-tetrahydro-2H-pyrano[3,4-c]isoquinolin-1-yl)-N-methylindolizine-2-carboxamide;
2-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
3-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
N-(6-((2-aminoethyl)amino)-8,9-difluoro-1,4-dihydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-6-fluoro-4-(1-hydroxyethyl)-N-methyl-1H-indole-2-carboxamide;
N-(8,9-difluoro-6-oxo-1,4,5,6-tetrahydro-2H-pyrano[3,4-c]isoquinolin-1-yl)-6-fluoro-4-(hydroxymethyl)-N-methyl-1H-indole-2-carboxamide;
7-bromo-N-(8,9-difluoro-6-oxo-1,4,5,6-tetrahydro-2H-pyrano[3,4-c]isoquinolin-1-yl)-N-methylfuro[3,2-c]pyridine-2-carboxamide;
3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-6-carboxamide;
3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-6-carboxamide;
3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-5-carboxamide; 3,4-dichloro-N-(8,9-difluoro-6-oxo-1,4,5,6-tetrahydro-2H-pyrano[3,4-c]isoquinolin-1-yl)-N-methylbenzamide;
N-(8,9-difluoro-6-oxo-1,4,5,6-tetrahydro-2H-pyrano[3,4-c]isoquinolin-1-yl)-6-fluoro-N-methyl-4-(methylsulfonamido)-1H-indole-2-carboxamide;
N-(8,9-difluoro-6-oxo-1,4,5,6-tetrahydro-2H-pyrano[3,4-c]isoquinolin-1-yl)-N-methyl-4-(trifluoromethoxy)-1H-indole-2-carboxamide;
N-(8,9-difluoro-6-oxo-1,4,5,6-tetrahydro-2H-pyrano[3,4-c]isoquinolin-1-yl)-6-methoxy-N-methyl-1H-indole-2-carboxamide;
5-bromo-N-(8,9-difluoro-6-oxo-1,4,5,6-tetrahydro-2H-pyrano[3,4-c]isoquinolin-1-yl)-N-methyl-6-(trifluoromethyl)nicotinamide;
3-chloro-N-(8,9-difluoro-6-oxo-1,4,5,6-tetrahydro-2H-pyrano[3,4-c]isoquinolin-1-yl)-N-methylindolizine-6-carboxamide;
N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-6-carboxamide;
N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-5-carboxamide;
N-(8,9-difluoro-6-oxo-1,4,5,6-tetrahydro-2H-pyrano[3,4-c]isoquinolin-1-yl)-3-methoxy-N-methyl-1H-indazole-5-carboxamide;
1-(5-chlorothiophen-3-yl)-N-(8,9-difluoro-6-oxo-1,4,5,6-tetrahydro-2H-pyrano[3,4-c]isoquinolin-1-yl)-N-methylazetidine-3-carboxamide;
7-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
4-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
3-chloro-N-(8,9-difluoro-6-oxo-1,4,5,6-tetrahydro-2H-pyrano[3,4-c]isoquinolin-1-yl)-N-methylindolizine-7-carboxamide;
N-(8,9-difluoro-6-oxo-1,4,5,6-tetrahydro-2H-pyrano[3,4-c]isoquinolin-1-yl)-7-(difluoromethyl)-N-methylindolizine-2-carboxamide;
N-(8,9-difluoro-6-oxo-1,4,5,6-tetrahydro-2H-pyrano[3,4-c]isoquinolin-1-yl)-6-(difluoromethyl)-N-methylindolizine-2-carboxamide;
di-tert-butyl ((2-((8,9-difluoro-6-oxo-1,4,5,6-tetrahydro-2H-pyrano[3,4-c]isoquinolin-1-yl)(methyl)carbamoyl)-5,6-difluoro-1H-indol-1-yl)methyl)phosphate;
5-chloro-N-(8,9-difluoro-6-oxo-1,4,5,6-tetrahydro-2H-pyrano[3,4-c]isoquinolin-1-yl)-N-methyl-6-(trifluoromethyl)nicotinamide;
5-chloro-N-(8-fluoro-6-oxo-1,4,5,6-tetrahydro-2H-pyrano[3,4-c]isoquinolin-1-yl)-N-methyl-6-(trifluoromethyl)nicotinamide;
N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
4-cyano-6-fluoro-N-(8-fluoro-6-oxo-1,4,5,6-tetrahydro-2H-pyrano[3,4-c]isoquinolin-1-yl)-N-methyl-1H-indole-2-carboxamide;
4-bromo-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
N-(8,9-difluoro-6-oxo-1,2,3,4,5,6-hexahydrobenzo[c][1,7]naphthyridin-1-yl)-7-(difluoromethyl)-N-methylindolizine-2-carboxamide;
N-(8,9-difluoro-6-oxo-1,2,3,4,5,6-hexahydrobenzo[c][1,7]naphthyridin-1-yl)-6-(difluoromethyl)-N-methylindolizine-2-carboxamide;
N-(8-fluoro-6-oxo-1,4,5,6-tetrahydro-2H-pyrano[3,4-c]isoquinolin-1-yl)-N-methylindolizine-6-carboxamide;
N-(8,9-difluoro-6-oxo-1,2,3,4,5,6-hexahydrobenzo[c][1,7]naphthyridin-1-yl)-N-methylindolizine-6-carboxamide;
4-ethyl-6-fluoro-N-(8-fluoro-6-oxo-1,4,5,6-tetrahydro-2H-pyrano[3,4-c]isoquinolin-1-yl)-N-methyl-1H-indole-2-carboxamide;
5-cyano-N-(8-fluoro-6-oxo-1,4,5,6-tetrahydro-2H-pyrano[3,4-c]isoquinolin-1-yl)-N-methyl-1H-indole-2-carboxamide;
4-cyano-N-(8,9-difluoro-6-oxo-1,2,3,4,5,6-hexahydrobenzo[c][1,7]naphthyridin-1-yl)-6-fluoro-N-methyl-1H-indole-2-carboxamide;
N-(8,9-difluoro-6-oxo-1,2,3,4,5,6-hexahydrobenzo[c][1,7]naphthyridin-1-yl)-4-ethyl-6-fluoro-N-methyl-1H-indole-2-carboxamide;
N-(8,9-difluoro-6-oxo-1,2,3,4,5,6-hexahydrobenzo[c][1,7]naphthyridin-1-yl)-N-methyl-5-(methylsulfonyl)-1H-indole-2-carboxamide;
5-cyano-N-(8,9-difluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-8-(difluoromethyl)-N-methylindolizine-2-carboxamide;
N-(8,9-difluoro-6-oxo-1,4,5,6-tetrahydro-2H-pyrano[3,4-c]isoquinolin-1-yl)-N-methyl-2,3-dihydro-1H-indene-5-carboxamide;
N-(8,9-difluoro-6-oxo-1,4,5,6-tetrahydro-2H-pyrano[3,4-c]isoquinolin-1-yl)-N-methylbenzo[d]oxazole-6-carboxamide;
4,6-dichloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
5,6-difluoro-N-methyl-N-(4,8,9-trifluoro-6-oxo-1,4,5,6-tetrahydro-2H-pyrano[3,4-c]isoquinolin-1-yl)-1H-indole-2-carboxamide;
N-(8,9-difluoro-6-oxo-1,4,5,6-tetrahydro-2H-pyrano[3,4-c]isoquinolin-1-yl)-5-fluoro-N-methyl-6-(trifluoromethyl)nicotinamide;
5-fluoro-N-(8-fluoro-6-oxo-1,4,5,6-tetrahydro-2H-pyrano[3,4-c]isoquinolin-1-yl)-N-methyl-6-(trifluoromethyl)nicotinamide;
3-chloro-N-(8,9-difluoro-6-oxo-1,4,5,6-tetrahydro-2H-pyrano[3,4-c]isoquinolin-1-yl)-N,1-dimethyl-1H-indazole-5-carboxamide;
3-chloro-N-(8,9-difluoro-6-oxo-1,4,5,6-tetrahydro-2H-pyrano[3,4-c]isoquinolin-1-yl)-1-ethyl-N-methyl-1H-indazole-5-carboxamide;
N-(8,9-difluoro-6-oxo-1,4,5,6-tetrahydro-2H-pyrano[3,4-c]isoquinolin-1-yl)-1-(difluoromethyl)-N,3-dimethyl-1H-indazole-5-carboxamide;
5-chloro-N-(8,9-difluoro-6-oxo-1,2,3,4,5,6-hexahydrobenzo[c][1,7]naphthyridin-1-yl)-N-methyl-6-(trifluoromethyl)nicotinamide;
N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
N-(8-fluoro-6-oxo-1,4,5,6-tetrahydro-2H-pyrano[3,4-c]isoquinolin-1-yl)-N,4-dimethyl-1H-indole-2-carboxamide;
4-chloro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methylbenzo[d]oxazole-5-carboxamide;
N-(8,9-difluoro-6-oxo-1,4,5,6-tetrahydro-2H-pyrano[3,4-c]isoquinolin-1-yl)-N-methylbenzo[d]thiazole-5-carboxamide;
N-(8,9-difluoro-6-oxo-1,4,5,6-tetrahydro-2H-pyrano[3,4-c]isoquinolin-1-yl)-N-methylbenzo[d]thiazole-6-carboxamide;
N-(8,9-difluoro-6-oxo-1,4,5,6-tetrahydro-2H-pyrano[3,4-c]isoquinolin-1-yl)-N-methyl-1H-indazole-5-carboxamide;
N-(8,9-difluoro-6-oxo-1,2,3,4,5,6-hexahydrobenzo[c][1,7]naphthyridin-1-yl)-8-(difluoromethyl)-N-methylindolizine-2-carboxamide;
N-(8,9-difluoro-6-oxo-1,2,3,4,5,6-hexahydrobenzo[c][1,7]naphthyridin-1-yl)-5-(difluoromethyl)-N-methylindolizine-2-carboxamide;
N-(8,9-difluoro-6-oxo-1,2,3,4,5,6-hexahydrobenzo[c][1,7]naphthyridin-1-yl)-5-fluoro-N-methyl-6-(trifluoromethyl)nicotinamide;
N-(8,9-difluoro-6-oxo-1,4,5,6-tetrahydro-2H-pyrano[3,4-c]isoquinolin-1-yl)-3-(difluoromethyl)-N,1-dimethyl-1H-indazole-5-carboxamide;
N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
4-chloro-6-fluoro-N-(8-fluoro-6-oxo-1,4,5,6-tetrahydro-2H-pyrano[3,4-c]isoquinolin-1-yl)-N-methyl-1H-indole-2-carboxamide;
4-chloro-N-(8,9-difluoro-6-oxo-1,2,3,4,5,6-hexahydrobenzo[c][1,7]naphthyridin-1-yl)-N-methyl-1H-indole-2-carboxamide;
N-(8-fluoro-6-oxo-1,4,5,6-tetrahydro-2H-pyrano[3,4-c]isoquinolin-1-yl)-N-methyl-2,3-dihydro-1H-indene-5-carboxamide;
N-(8-fluoro-6-oxo-1,4,5,6-tetrahydro-2H-pyrano[3,4-c]isoquinolin-1-yl)-N-methylbenzo[d]thiazole-5-carboxamide;
N-(8-fluoro-6-oxo-1,4,5,6-tetrahydro-2H-pyrano[3,4-c]isoquinolin-1-yl)-N-methylbenzo[d]thiazole-6-carboxamide;
N-(8-fluoro-6-oxo-1,4,5,6-tetrahydro-2H-pyrano[3,4-c]isoquinolin-1-yl)-N-methyl-1H-indazole-5-carboxamide;
N-(8,9-difluoro-6-oxo-1,4,5,6-tetrahydro-2H-pyrano[3,4-c]isoquinolin-1-yl)-N-methyl-1H-indazole-6-carboxamide;
N-(8,9-difluoro-6-oxo-1,4,5,6-tetrahydro-2H-pyrano[3,4-c]isoquinolin-1-yl)-N-methyl-1H-benzo[d]imidazole-6-carboxamide;
2-chloro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
2-chloro-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
6-fluoro-N-(8-fluoro-6-oxo-1,4,5,6-tetrahydro-2H-pyrano[3,4-c]isoquinolin-1-yl)-N,4-dimethyl-1H-indole-2-carboxamide;
N-(8,9-difluoro-6-oxo-1,2,3,4,5,6-hexahydrobenzo[c][1,7]naphthyridin-1-yl)-N,4-dimethyl-1H-indole-2-carboxamide;
N-(8,9-difluoro-6-oxo-1,2,3,4,5,6-hexahydrobenzo[c][1,7]naphthyridin-1-yl)-6-fluoro-N,4-dimethyl-1H-indole-2-carboxamide;
4-chloro-N-(8,9-difluoro-6-oxo-1,2,3,4,5,6-hexahydrobenzo[c][1,7]naphthyridin-1-yl)-6-fluoro-N-methyl-1H-indole-2-carboxamide;
N-(8,9-difluoro-6-oxo-1,2,3,4,5,6-hexahydrobenzo[c][1,7]naphthyridin-1-yl)-N-methylbenzo[d]thiazole-5-carboxamide;
N-(8,9-difluoro-6-oxo-1,2,3,4,5,6-hexahydrobenzo[c][1,7]naphthyridin-1-yl)-N-methylbenzo[d]thiazole-6-carboxamide;
N-(8,9-difluoro-6-oxo-1,2,3,4,5,6-hexahydrophenanthridin-1-yl)-N-methyl-1H-indole-2-carboxamide;
2-cyano-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
N-(8-fluoro-6-oxo-1,4,5,6-tetrahydro-2H-pyrano[3,4-c]isoquinolin-1-yl)-N-methyl-1H-indazole-6-carboxamide;
N-(8,9-difluoro-6-oxo-1,2,3,4,5,6-hexahydrobenzo[c][1,7]naphthyridin-1-yl)-N-methyl-2,3-dihydro-1H-indene-5-carboxamide;
N-(8,9-difluoro-6-oxo-1,2,3,4,5,6-hexahydrobenzo[c][1,7]naphthyridin-1-yl)-N-methyl-1H-indazole-5-carboxamide;
N-(8-fluoro-6-oxo-1,2,3,4,5,6-hexahydrophenanthridin-1-yl)-N-methyl-1H-indole-2-carboxamide;
2-chloro-N-(8,9-difluoro-4,6-dioxo-1,4,5,6-tetrahydro-2H-pyrano[3,4-c]isoquinolin-1-yl)-N-methyl-4H-thieno[3,2-b]pyrrole-5-carboxamide;
N-(8,9-difluoro-4,6-dioxo-1,4,5,6-tetrahydro-2H-pyrano[3,4-c]isoquinolin-1-yl)-6-(difluoromethyl)-5-fluoro-N-methyl-1H-indole-2-carboxamide;
6-(difluoromethyl)-5-fluoro-N-(8-fluoro-6-oxo-1,2,3,4,5,6-hexahydrophenanthridin-1-yl)-N-methyl-1H-indole-2-carboxamide;
N-(8,9-difluoro-6-oxo-1,2,3,4,5,6-hexahydrophenanthridin-1-yl)-5-fluoro-N-methyl-1H-indole-2-carboxamide;
N-(8,9-difluoro-6-oxo-1,2,3,4,5,6-hexahydrophenanthridin-1-yl)-5,6-difluoro-N-methyl-1H-indole-2-carboxamide;
N-(8,9-difluoro-6-oxo-1,2,3,4,5,6-hexahydrophenanthridin-1-yl)-6-(difluoromethyl)-5-fluoro-N-methyl-1H-indole-2-carboxamide;
N-(8,9-difluoro-6-oxo-1,2,3,4,5,6-hexahydrophenanthridin-1-yl)-4-(difluoromethyl)-6-fluoro-N-methyl-1H-indole-2-carboxamide;
N-(8,9-difluoro-6-oxo-1,2,3,4,5,6-hexahydrophenanthridin-1-yl)-N,4-dimethyl-1H-indole-2-carboxamide;
N-(8,9-difluoro-6-oxo-1,2,3,4,5,6-hexahydrophenanthridin-1-yl)-6-fluoro-N,4-dimethyl-1H-indole-2-carboxamide;
1-((4-bromo-3-fluorobenzyl)(methyl)amino)-8,9-difluoro-1,5-dihydro-2H-pyrano[3,4-c]isoquinolin-6(4H)-one;
2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
5-fluoro-N-(8-fluoro-6-oxo-1,2,3,4,5,6-hexahydrophenanthridin-1-yl)-N-methyl-1H-indole-2-carboxamide;
5,6-difluoro-N-(8-fluoro-6-oxo-1,2,3,4,5,6-hexahydrophenanthridin-1-yl)-N-methyl-1H-indole-2-carboxamide;
N-(8-fluoro-6-oxo-1,2,3,4,5,6-hexahydrophenanthridin-1-yl)-N,4-dimethyl-1H-indole-2-carboxamide;
6-fluoro-N-(8-fluoro-6-oxo-1,2,3,4,5,6-hexahydrophenanthridin-1-yl)-N,4-dimethyl-1H-indole-2-carboxamide;
4-(difluoromethyl)-6-fluoro-N-(8-fluoro-6-oxo-1,2,3,4,5,6-hexahydrophenanthridin-1-yl)-N-methyl-1H-indole-2-carboxamide;
1-(((5,6-difluoro-1H-indol-2-yl)methyl)(methyl)amino)-8,9-difluoro-1,5-dihydro-2H-pyrano[3,4-c]isoquinolin-6(4H)-one;
N-(8,9-difluoro-6-oxo-1,2,3,4,5,6-hexahydrobenzo[c][1,7]naphthyridin-1-yl)-N-methyl-1H-indazole-6-carboxamide;
2-fluoro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
N-(8,9-difluoro-6-oxo-1,2,3,4,5,6-hexahydrobenzo[c][1,7]naphthyridin-1-yl)-2-fluoro-N-methyl-4H-thieno[3,2-b]pyrrole-5-carboxamide;
N-(8,9-difluoro-4-hydroxy-6-oxo-1,2,3,4,5,6-hexahydrophenanthridin-1-yl)-5-fluoro-N-methyl-1H-indole-2-carboxamide;
N-(8,9-difluoro-4-hydroxy-6-oxo-1,2,3,4,5,6-hexahydrophenanthridin-1-yl)-5,6-difluoro-N-methyl-1H-indole-2-carboxamide;
N-(8,9-difluoro-4-hydroxy-6-oxo-1,2,3,4,5,6-hexahydrophenanthridin-1-yl)-N-methyl-1H-indole-2-carboxamide;
N-(8,9-difluoro-6-oxo-1,2,3,4,5,6-hexahydrobenzo[c][1,7]naphthyridin-1-yl)-N-methylindolizine-2-carboxamide;
8-chloro-N-(8,9-difluoro-6-oxo-1,2,3,4,5,6-hexahydrobenzo[c][1,7]naphthyridin-1-yl)-N-methylindolizine-2-carboxamide;
N-(8,9-Difluoro-6-oxo-1,4,5,6-tetrahydro-2H-pyrano[3,4-c]isoquinolin-1-yl)-2,2-difluoro-N-methyl-2-phenylacetamide;
N-(8,9-Difluoro-6-oxo-1,4,5,6-tetrahydro-2H-pyrano[3,4-c]isoquinolin-1-yl-1-d)-5,6-difluoro-N-(methyl-13C-d₃)-1H-indole-2-carboxamide;
N-(8,9-Difluoro-6-oxo-1,4,5,6-tetrahydro-2H-pyrano[3,4-c]isoquinolin-1-yl-1-d)-6-(difluoromethyl)-5-fluoro-N-(methyl-13C-d₃)-1H-indole-2-carboxamide; and
2-Chloro-N-(8,9-difluoro-6-oxo-1,4,5,6-tetrahydro-2H-pyrano[3,4-c]isoquinolin-1-yl-1-d)-N-(methyl-13C-d₃)-4H-thieno[3,2-b]pyrrole-5-carboxamide;
   or a salt, solvate, prodrug, isotopically labelled derivative, stereoisomer, or tautomer thereof, or any mixtures thereof.

Embodiment 13 provides the compound of any of Embodiments 1-12, which is at least one selected from the group consisting of:
(R)-N-(8-fluoro-6-oxo-1,4,5,6-tetrahydro-2H-pyrano[3,4-c]isoquinolin-1-yl)-N-methyl-1H-indole-2-carboxamide;
(S)-N-(8-fluoro-6-oxo-1,4,5,6-tetrahydro-2H-pyrano[3,4-c]isoquinolin-1-yl)-N-methyl-1H-indole-2-carboxamide;
(2S)-N-(8-fluoro-6-oxo-1,4,5,6-tetrahydro-2H-pyrano[3,4-c]isoquinolin-1-yl)-N-methylindoline-2-carboxamide;
(2S)-N-(8-fluoro-6-oxo-1,4,5,6-tetrahydro-2H-pyrano[3,4-c]isoquinolin-(1R)-yl)-N-methylindoline-2-carboxamide;
(2S)-N-(8-fluoro-6-oxo-1,4,5,6-tetrahydro-2H-pyrano[3,4-c]isoquinolin-(1S)-yl)-N-methylindoline-2-carboxamide;
(2R)-N-(8-fluoro-6-oxo-1,4,5,6-tetrahydro-2H-pyrano[3,4-c]isoquinolin-1-yl)-N-methylindoline-2-carboxamide;
(2R)-N-(8-fluoro-6-oxo-1,4,5,6-tetrahydro-2H-pyrano[3,4-c]isoquinolin-(1R)-yl)-N-methylindoline-2-carboxamide;
(2R)-N-(8-fluoro-6-oxo-1,4,5,6-tetrahydro-2H-pyrano[3,4-c]isoquinolin-(1S)-yl)-N-methylindoline-2-carboxamide;
(N-(8,9-difluoro-6-oxo-1,4,5,6-tetrahydro-2H-pyrano[3,4-c]isoquinolin-(1R)-yl)-5-fluoro-N-methylindoline-(2R)-carboxamide;
N-(8,9-difluoro-6-oxo-1,4,5,6-tetrahydro-2H-pyrano[3,4-c]isoquinolin-(1S)-yl)-5-fluoro-N-methylindoline-(2R)-carboxamide;
N-(8,9-difluoro-6-oxo-1,4,5,6-tetrahydro-2H-pyrano[3,4-c]isoquinolin-(1R)-yl)-5-fluoro-N-methylindoline-(2S)-carboxamide;
N-(8,9-difluoro-6-oxo-1,4,5,6-tetrahydro-2H-pyrano[3,4-c]isoquinolin-(1S)-yl)-5-fluoro-N-methylindoline-(2S)-carboxamide;
(R)-N-(8,9-difluoro-6-oxo-1,4,5,6-tetrahydro-2H-pyrano[3,4-c]isoquinolin-1-yl)-8-fluoro-N-methylindolizine-2-carboxamide;
(S)-N-(8,9-difluoro-6-oxo-1,4,5,6-tetrahydro-2H-pyrano[3,4-c]isoquinolin-1-yl)-8-fluoro-N-methylindolizine-2-carboxamide;
(R)-N-(8,9-difluoro-6-oxo-1,4,5,6-tetrahydro-2H-pyrano[3,4-c]isoquinolin-1-yl)-N-methylindolizine-2-carboxamide;
(S)-N-(8,9-difluoro-6-oxo-1,4,5,6-tetrahydro-2H-pyrano[3,4-c]isoquinolin-1-yl)-N-methylindolizine-2-carboxamide;
N-(8,9-difluoro-6-oxo-1,4,5,6-tetrahydro-2H-pyrano[3,4-c]isoquinolin-(1R)-yl)-4,6-difluoro-N-methylindoline-(2R)-carboxamide;
N-(8,9-difluoro-6-oxo-1,4,5,6-tetrahydro-2H-pyrano[3,4-c]isoquinolin-(1R)-yl)-4,6-difluoro-N-methylindoline-(2S)-carboxamide;
N-(8,9-difluoro-6-oxo-1,4,5,6-tetrahydro-2H-pyrano[3,4-c]isoquinolin-(1S)-yl)-4,6-difluoro-N-methylindoline-(2R)-carboxamide;
N-(8,9-difluoro-6-oxo-1,4,5,6-tetrahydro-2H-pyrano[3,4-c]isoquinolin-(1S)-yl)-4,6-difluoro-N-methylindoline-(2S)-carboxamide;
(S)-N-((S)-8,9-difluoro-6-oxo-1,4,5,6-tetrahydro-2H-pyrano[3,4-c]isoquinolin-1-yl)-4,6-difluoro-N-methylindoline-2-carboxamide;
(S)-N-((R)-8,9-difluoro-6-oxo-1,4,5,6-tetrahydro-2H-pyrano[3,4-c]isoquinolin-1-yl)-4,6-difluoro-N-methylindoline-2-carboxamide;
(R)-N-((S)-8,9-difluoro-6-oxo-1,4,5,6-tetrahydro-2H-pyrano[3,4-c]isoquinolin-1-yl)-4,6-difluoro-N-methylindoline-2-carboxamide;
(S)-N-((R)-8,9-difluoro-6-oxo-1,4,5,6-tetrahydro-2H-pyrano[3,4-c]isoquinolin-1-yl)-4,6-difluoro-N-methylindoline-2-carboxamide;
(R)-8-chloro-N-(8,9-difluoro-6-oxo-1,4,5,6-tetrahydro-2H-pyrano[3,4-c]isoquinolin-1-yl)-N-methylindolizine-2-carboxamide;
(S)-8-chloro-N-(8,9-difluoro-6-oxo-1,4,5,6-tetrahydro-2H-pyrano[3,4-c]isoquinolin-1-yl)-N-methylindolizine-2-carboxamide;
(R)-4-fluoro-N-(8-fluoro-6-oxo-1,4,5,6-tetrahydro-2H-pyrano[3,4-c]isoquinolin-1-yl)-N-methyl-1H-indole-2-carboxamide;
(S)-4-fluoro-N-(8-fluoro-6-oxo-1,4,5,6-tetrahydro-2H-pyrano[3,4-c]isoquinolin-1-yl)-N-methyl-1H-indole-2-carboxamide;
(R)-N-(8,9-difluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-1H-indole-2-carboxamide;
(R)-N-(8,9-difluoro-6-oxo-1,4,5,6-tetrahydro-2H-pyrano[3,4-c]isoquinolin-1-yl)-4-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-4-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-5-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-5-fluoro-N-methyl-1H-indole-2-carboxamide;
(R)-5-fluoro-N-(8-fluoro-6-oxo-1,4,5,6-tetrahydro-2H-pyrano[3,4-c]isoquinolin-1-yl)-N-methyl-1H-indole-2-carboxamide;
(S)-5-fluoro-N-(8-fluoro-6-oxo-1,4,5,6-tetrahydro-2H-pyrano[3,4-c]isoquinolin-1-yl)-N-methyl-1H-indole-2-carboxamide;
(R)-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-7-fluoro-N-(8-fluoro-6-oxo-1,4,5,6-tetrahydro-2H-pyrano[3,4-c]isoquinolin-1-yl)-N-methyl-1H-indole-2-carboxamide;
(S)-7-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4,6-difluoro-N-(8-fluoro-6-oxo-1,4,5,6-tetrahydro-2H-pyrano[3,4-c]isoquinolin-1-yl)-N-methyl-1H-indole-2-carboxamide;
(S)-4,6-difluoro-N-(8-fluoro-6-oxo-1,4,5,6-tetrahydro-2H-pyrano[3,4-c]isoquinolin-1-yl)-N-methyl-1H-indole-2-carboxamide;
(R)-4,5-difluoro-N-(8-fluoro-6-oxo-1,4,5,6-tetrahydro-2H-pyrano[3,4-c]isoquinolin-1-yl)-N-methyl-1H-indole-2-carboxamide;
(S)-4,5-difluoro-N-(8-fluoro-6-oxo-1,4,5,6-tetrahydro-2H-pyrano[3,4-c]isoquinolin-1-yl)-N-methyl-1H-indole-2-carboxamide;
(R)-N-(8,9-difluoro-6-oxo-1,4,5,6-tetrahydro-2H-pyrano[3,4-c]isoquinolin-1-yl)-7-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-7-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-5,6-difluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-4,6-difluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4,5-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-4,5-difluoro-N-methyl-1H-indole-2-carboxamide;
(R)-N-(8,9-difluoro-6-oxo-1,4,5,6-tetrahydro-2H-pyrano[3,4-c]isoquinolin-1-yl)-5,5-difluoro-N-methyl-4,5,6,7-tetrahydro-1H-indole-2-carboxamide;
(S)-N-(8,9-difluoro-6-oxo-1,4,5,6-tetrahydro-2H-pyrano[3,4-c]isoquinolin-1-yl)-5,5-difluoro-N-methyl-4,5,6,7-tetrahydro-1H-indole-2-carboxamide;
(R)-5,6-difluoro-N-(8-fluoro-6-oxo-1,4,5,6-tetrahydro-2H-pyrano[3,4-c]isoquinolin-1-yl)-N-methyl-1H-indole-2-carboxamide;
(S)-5,6-difluoro-N-(8-fluoro-6-oxo-1,4,5,6-tetrahydro-2H-pyrano[3,4-c]isoquinolin-1-yl)-N-methyl-1H-indole-2-carboxamide;
(R)-6-chloro-N-(8,9-difluoro-6-oxo-1,4,5,6-tetrahydro-2H-pyrano[3,4-c]isoquinolin-1-yl)-N-methylimidazo[1,2-a]pyridine-2-carboxamide;
(S)-6-chloro-N-(8,9-difluoro-6-oxo-1,4,5,6-tetrahydro-2H-pyrano[3,4-c]isoquinolin-1-yl)-N-methylimidazo[1,2-a]pyridine-2-carboxamide;
(R)-6-chloro-N-(8-fluoro-6-oxo-1,4,5,6-tetrahydro-2H-pyrano[3,4-c]isoquinolin-1-yl)-N-methylimidazo[1,2-a]pyridine-2-carboxamide;
(S)-6-chloro-N-(8-fluoro-6-oxo-1,4,5,6-tetrahydro-2H-pyrano[3,4-c]isoquinolin-1-yl)-N-methylimidazo[1,2-a]pyridine-2-carboxamide;
(R)-6-chloro-N-(8-fluoro-6-oxo-1,4,5,6-tetrahydro-2H-pyrano[3,4-c]isoquinolin-1-yl)-N-ethylimidazo[1,2-a]pyridine-2-carboxamide;
(S)-6-chloro-N-(8-fluoro-6-oxo-1,4,5,6-tetrahydro-2H-pyrano[3,4-c]isoquinolin-1-yl)-N-ethylimidazo[1,2-a]pyridine-2-carboxamide;
(R)-N-(8,9-difluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-(1R)-yl)-N,3,3-trimethylindoline-(2R)-carboxamide;
N-(8,9-difluoro-6-oxo-1,4,5,6-tetrahydro-2H-pyrano[3,4-c]isoquinolin-(1R)-yl)-N,3,3-trimethylindoline-(2S)-carboxamide;
N-(8,9-difluoro-6-oxo-1,4,5,6-tetrahydro-2H-pyrano[3,4-c]isoquinolin-(1S)-yl)-N,3,3-trimethylindoline-(2R)-carboxamide;
N-(8,9-difluoro-6-oxo-1,4,5,6-tetrahydro-2H-pyrano[3,4-c]isoquinolin-(1S)-yl)-N,3,3-trimethylindoline-(2S)-carboxamide;
(R)-N-(8-fluoro-3-methyl-6-oxo-1,2,3,4,5,6-hexahydrobenzo[c][1,7]naphthyridin-1-yl)-N-methyl-1H-indole-2-carboxamide;
(S)-N-(8-fluoro-3-methyl-6-oxo-1,2,3,4,5,6-hexahydrobenzo[c][1,7]naphthyridin-1-yl)-N-methyl-1H-indole-2-carboxamide;
(R)-N-(8,9-difluoro-3-methyl-6-oxo-1,2,3,4,5,6-hexahydrobenzo[c][1,7]naphthyridin-1-yl)-N-methyl-1H-indole-2-carboxamide;
(S)-N-(8,9-difluoro-3-methyl-6-oxo-1,2,3,4,5,6-hexahydrobenzo[c][1,7]naphthyridin-1-yl)-N-methyl-1H-indole-2-carboxamide;
(R)-N-(8,9-difluoro-6-oxo-1,4,5,6-tetrahydro-2H-pyrano[3,4-c]isoquinolin-1-yl)-N-methylpyrazolo[1,5-a]pyridine-2-carboxamide;
(S)-N-(8,9-difluoro-6-oxo-1,4,5,6-tetrahydro-2H-pyrano[3,4-c]isoquinolin-1-yl)-N-methylpyrazolo[1,5-a]pyridine-2-carboxamide;
(R)-N-(8,9-difluoro-6-oxo-1,4,5,6-tetrahydro-2H-pyrano[3,4-c]isoquinolin-1-yl)-N-methylpyrrolo[1,2-b]pyridazine-6-carboxamide;
(S)-N-(8,9-difluoro-6-oxo-1,4,5,6-tetrahydro-2H-pyrano[3,4-c]isoquinolin-1-yl)-N-methylpyrrolo[1,2-b]pyridazine-6-carboxamide;
(R)-N-(8,9-difluoro-6-oxo-1,4,5,6-tetrahydro-2H-pyrano[3,4-c]isoquinolin-1-yl)-N-methylquinoline-7-carboxamide;
(S)-N-(8,9-difluoro-6-oxo-1,4,5,6-tetrahydro-2H-pyrano[3,4-c]isoquinolin-1-yl)-N-methylquinoline-7-carboxamide;
(R)-N-(8,9-difluoro-6-oxo-1,4,5,6-tetrahydro-2H-pyrano[3,4-c]isoquinolin-1-yl)-N-methylquinoline-6-carboxamide;
(S)-N-(8,9-difluoro-6-oxo-1,4,5,6-tetrahydro-2H-pyrano[3,4-c]isoquinolin-1-yl)-N-methylquinoline-6-carboxamide;
(R)-N-(3-Acetyl-8-fluoro-6-oxo-1,2,3,4,5,6-hexahydrobenzo[c][1,7]naphthyridin-1-yl)-N-methyl-1H-indole-2-carboxamide;
(S)-N-(3-Acetyl-8-fluoro-6-oxo-1,2,3,4,5,6-hexahydrobenzo[c][1,7]naphthyridin-1-yl)-N-methyl-1H-indole-2-carboxamide;
(R)-N-(8,9-Difluoro-6-oxo-1,4,5,6-tetrahydro-2H-pyrano[3,4-c]isoquinolin-1-yl)-N-methyl-[1,2,4]triazolo[4,3-a]pyridine-6-carboxamide;
(S)-N-(8,9-Difluoro-6-oxo-1,4,5,6-tetrahydro-2H-pyrano[3,4-c]isoquinolin-1-yl)-N-methyl-[1,2,4]triazolo[4,3-a]pyridine-6-carboxamide;
(R)-N-(8,9-Difluoro-6-oxo-1,4,5,6-tetrahydro-2H-pyrano[3,4-c]isoquinolin-1-yl)-N-methylquinoline-3-carboxamide;
(S)-N-(8,9-Difluoro-6-oxo-1,4,5,6-tetrahydro-2H-pyrano[3,4-c]isoquinolin-1-yl)-N-methylquinoline-3-carboxamide;
(R)-N-(8,9-Difluoro-6-oxo-1,4,5,6-tetrahydro-2H-pyrano[3,4-c]isoquinolin-1-yl)-N-methylquinoxaline-6-carboxamide;
(S)-N-(8,9-Difluoro-6-oxo-1,4,5,6-tetrahydro-2H-pyrano[3,4-c]isoquinolin-1-yl)-N-methylquinoxaline-6-carboxamide;
(R)-N-(8,9-Difluoro-6-oxo-1,4,5,6-tetrahydro-2H-pyrano[3,4-c]isoquinolin-1-yl)-N-methyl-6-(trifluoromethyl)nicotinamide;
(S)-N-(8,9-Difluoro-6-oxo-1,4,5,6-tetrahydro-2H-pyrano[3,4-c]isoquinolin-1-yl)-N-methyl-6-(trifluoromethyl)nicotinamide;
(R)-N-(8,9-Difluoro-6-oxo-1,4,5,6-tetrahydro-2H-pyrano[3,4-c]isoquinolin-1-yl)-3-fluoro-N-methyl-4-(trifluoromethyl)benzamide;
(S)-N-(8,9-Difluoro-6-oxo-1,4,5,6-tetrahydro-2H-pyrano[3,4-c]isoquinolin-1-yl)-3-fluoro-N-methyl-4-(trifluoromethyl)benzamide;
(R)-4-Chloro-N-(8,9-difluoro-6-oxo-1,4,5,6-tetrahydro-2H-pyrano[3,4-c]isoquinolin-1-yl)-3-fluoro-N-methylbenzamide;
(S)-4-Chloro-N-(8,9-difluoro-6-oxo-1,4,5,6-tetrahydro-2H-pyrano[3,4-c]isoquinolin-1-yl)-3-fluoro-N-methylbenzamide;
(R)-N-(8,9-Difluoro-6-oxo-1,4,5,6-tetrahydro-2H-pyrano[3,4-c]isoquinolin-1-yl)-3,4,5-trifluoro-N-methylbenzamide;
(S)-N-(8,9-Difluoro-6-oxo-1,4,5,6-tetrahydro-2H-pyrano[3,4-c]isoquinolin-1-yl)-3,4,5-trifluoro-N-methylbenzamide;
(R)-N-(8-Fluoro-6-oxo-1,2,3,4,5,6-hexahydrobenzo[c][1,7]naphthyridin-1-yl)-N-methyl-1H-indole-2-carboxamide;
(S)-N-(8-Fluoro-6-oxo-1,2,3,4,5,6-hexahydrobenzo[c][1,7]naphthyridin-1-yl)-N-methyl-1H-indole-2-carboxamide;
(R)-N-(8-Fluoro-3-(2-hydroxyethyl)-6-oxo-1,2,3,4,5,6-hexahydrobenzo[c][1,7]naphthyridin-1-yl)-N-methyl-1H-indole-2-carboxamide;
(S)-N-(8-Fluoro-3-(2-hydroxyethyl)-6-oxo-1,2,3,4,5,6-hexahydrobenzo[c][1,7]naphthyridin-1-yl)-N-methyl-1H-indole-2-carboxamide;
(R)-N-(3-Acetyl-8,9-difluoro-6-oxo-1,2,3,4,5,6-hexahydrobenzo[c][1,7]naphthyridin-1-yl)-N-methyl-1H-indole-2-carboxamide;
(S)-N-(3-Acetyl-8,9-difluoro-6-oxo-1,2,3,4,5,6-hexahydrobenzo[c][1,7]naphthyridin-1-yl)-N-methyl-1H-indole-2-carboxamide;
(R)-N-(8,9-Difluoro-6-oxo-1,4,5,6-tetrahydro-2H-pyrano[3,4-c]isoquinolin-1-yl)-5-fluoro-N-methylnicotinamide;
(S)-N-(8,9-Difluoro-6-oxo-1,4,5,6-tetrahydro-2H-pyrano[3,4-c]isoquinolin-1-yl)-5-fluoro-N-methylnicotinamide;
(R)-5-Chloro-N-(8,9-difluoro-6-oxo-1,4,5,6-tetrahydro-2H-pyrano[3,4-c]isoquinolin-1-yl)-N-methylnicotinamide;
(S)-5-Chloro-N-(8,9-difluoro-6-oxo-1,4,5,6-tetrahydro-2H-pyrano[3,4-c]isoquinolin-1-yl)-N-methylnicotinamide;
(R)-N-(8,9-Difluoro-6-oxo-1,4,5,6-tetrahydro-2H-pyrano[3,4-c]isoquinolin-1-yl)-2-fluoro-N-methylisonicotinamide;
(S)-N-(8,9-Difluoro-6-oxo-1,4,5,6-tetrahydro-2H-pyrano[3,4-c]isoquinolin-1-yl)-2-fluoro-N-methylisonicotinamide;
(R)-N-(8,9-Difluoro-6-oxo-1,4,5,6-tetrahydro-2H-pyrano[3,4-c]isoquinolin-1-yl)-3-(difluoromethyl)-N-methylbenzamide;
(S)-N-(8,9-Difluoro-6-oxo-1,4,5,6-tetrahydro-2H-pyrano[3,4-c]isoquinolin-1-yl)-3-(difluoromethyl)-N-methylbenzamide;
N-((S)-8,9-Difluoro-6-oxo-1,4,5,6-tetrahydro-2H-pyrano[3,4-c]isoquinolin-1-yl)-(2R)-hydroxy-N-methyl-3-phenylpropanamide;
N-((S)-8,9-Difluoro-6-oxo-1,4,5,6-tetrahydro-2H-pyrano[3,4-c]isoquinolin-1-yl)-(2S)-hydroxy-N-methyl-3-phenylpropanamide;
(R)-N-(8,9-Difluoro-3-(2-hydroxyethyl)-6-oxo-1,2,3,4,5,6-hexahydrobenzo[c][1,7]naphthyridin-1-yl)-N-methyl-1H-indole-2-carboxamide;
(S)-N-(8,9-Difluoro-3-(2-hydroxyethyl)-6-oxo-1,2,3,4,5,6-hexahydrobenzo[c][1,7]naphthyridin-1-yl)-N-methyl-1H-indole-2-carboxamide;
(R)-N-(8,9-Difluoro-6-oxo-1,4,5,6-tetrahydro-2H-pyrano[3,4-c]isoquinolin-1-yl)-N-methylbenzamide;
(S)-N-(8,9-Difluoro-6-oxo-1,4,5,6-tetrahydro-2H-pyrano[3,4-c]isoquinolin-1-yl)-N-methylbenzamide;
(R)-N-(8,9-Difluoro-6-oxo-1,4,5,6-tetrahydro-2H-pyrano[3,4-c]isoquinolin-1-yl)-3,4-difluoro-N-methylbenzamide;
(S)-N-(8,9-Difluoro-6-oxo-1,4,5,6-tetrahydro-2H-pyrano[3,4-c]isoquinolin-1-yl)-3,4-difluoro-N-methylbenzamide;
N-((S)-8,9-Difluoro-6-oxo-1,4,5,6-tetrahydro-2H-pyrano[3,4-c]isoquinolin-1-yl)-(2R)-hydroxy-N-methyl-2-phenylpropanamide;
N-((S)-8,9-Difluoro-6-oxo-1,4,5,6-tetrahydro-2H-pyrano[3,4-c]isoquinolin-1-yl)-(2S)-hydroxy-N-methyl-2-phenylpropanamide;
N-((R)-8,9-Difluoro-6-oxo-1,4,5,6-tetrahydro-2H-pyrano[3,4-c]isoquinolin-1-yl)-(2R)-hydroxy-N-methyl-2-phenylpropanamide;
N-((R)-8,9-Difluoro-6-oxo-1,4,5,6-tetrahydro-2H-pyrano[3,4-c]isoquinolin-1-yl)-(2S)-hydroxy-N-methyl-2-phenylpropanamide; N-((S)-8,9-Difluoro-6-oxo-1,4,5,6-tetrahydro-2H-pyrano[3,4-c]isoquinolin-1-yl)-(2R)-hydroxy-N-methyl-2-phenylacetamide;
N-((S)-8,9-Difluoro-6-oxo-1,4,5,6-tetrahydro-2H-pyrano[3,4-c]isoquinolin-1-yl)-(2S)-hydroxy-N-methyl-2-phenylacetamide;
N-((R)-8,9-Difluoro-6-oxo-1,4,5,6-tetrahydro-2H-pyrano[3,4-c]isoquinolin-1-yl)-(2R)-hydroxy-N-methyl-2-phenylacetamide;
N-((R)-8,9-Difluoro-6-oxo-1,4,5,6-tetrahydro-2H-pyrano[3,4-c]isoquinolin-1-yl)-(2S)-hydroxy-N-methyl-2-phenylacetamide;
(R)-N-(8,9-Difluoro-6-oxo-1,4,5,6-tetrahydro-2H-pyrano[3,4-c]isoquinolin-1-yl)-4,5,6-trifluoro-N-methyl-1H-indole-2-carboxamide;
(S)-N-(8,9-Difluoro-6-oxo-1,4,5,6-tetrahydro-2H-pyrano[3,4-c]isoquinolin-1-yl)-4,5,6-trifluoro-N-methyl-1H-indole-2-carboxamide;
(R)-N-(8,9-Difluoro-6-oxo-1,4,5,6-tetrahydro-2H-pyrano[3,4-c]isoquinolin-1-yl)-N-methyl-4-(trifluoromethyl)benzamide;
(S)-N-(8,9-Difluoro-6-oxo-1,4,5,6-tetrahydro-2H-pyrano[3,4-c]isoquinolin-1-yl)-N-methyl-4-(trifluoromethyl)benzamide;
(R)-4-Chloro-N-(8,9-difluoro-6-oxo-1,4,5,6-tetrahydro-2H-pyrano[3,4-c]isoquinolin-1-yl)-N-methylbenzamide;
(S)-4-Chloro-N-(8,9-difluoro-6-oxo-1,4,5,6-tetrahydro-2H-pyrano[3,4-c]isoquinolin-1-yl)-N-methylbenzamide;
(R)-4-Cyano-N-(8,9-difluoro-6-oxo-1,4,5,6-tetrahydro-2H-pyrano[3,4-c]isoquinolin-1-yl)-3-fluoro-N-methylbenzamide;
(S)-4-Cyano-N-(8,9-difluoro-6-oxo-1,4,5,6-tetrahydro-2H-pyrano[3,4-c]isoquinolin-1-yl)-3-fluoro-N-methylbenzamide;
(R)-N-(8,9-Difluoro-6-oxo-1,2,3,4,5,6-hexahydrobenzo[c][1,7]naphthyridin-1-yl)-5-fluoro-N-methyl-1H-indole-2-carboxamide;
(S)-N-(8,9-Difluoro-6-oxo-1,2,3,4,5,6-hexahydrobenzo[c][1,7]naphthyridin-1-yl)-5-fluoro-N-methyl-1H-indole-2-carboxamide;
(R)-N-(8,9-Difluoro-6-oxo-1,4,5,6-tetrahydro-2H-thiopyrano[3,4-c]isoquinolin-1-yl)-8-fluoro-N-methylindolizine-2-carboxamide;
(S)-N-(8,9-Difluoro-6-oxo-1,4,5,6-tetrahydro-2H-thiopyrano[3,4-c]isoquinolin-1-yl)-8-fluoro-N-methylindolizine-2-carboxamide;
(R)-N-(8,9-Difluoro-6-oxo-1,2,3,4,5,6-hexahydrobenzo[c][1,7]naphthyridin-1-yl)-4-fluoro-N-methyl-1H-indole-2-carboxamide;
(S)-N-(8,9-Difluoro-6-oxo-1,2,3,4,5,6-hexahydrobenzo[c][1,7]naphthyridin-1-yl)-4-fluoro-N-methyl-1H-indole-2-carboxamide;
(R)-N-(8,9-Difluoro-6-oxo-1,2,3,4,5,6-hexahydrobenzo[c][1,7]naphthyridin-1-yl)-6-fluoro-N-methyl-1H-indole-2-carboxamide;
(S)-N-(8,9-Difluoro-6-oxo-1,2,3,4,5,6-hexahydrobenzo[c][1,7]naphthyridin-1-yl)-6-fluoro-N-methyl-1H-indole-2-carboxamide;
(R)-N-(8,9-Difluoro-6-oxo-1,4,5,6-tetrahydro-2H-pyrano[3,4-c]isoquinolin-1-yl)-3-(difluoromethyl)-4-fluoro-N-methylbenzamide;
(S)-N-(8,9-Difluoro-6-oxo-1,4,5,6-tetrahydro-2H-pyrano[3,4-c]isoquinolin-1-yl)-3-(difluoromethyl)-4-fluoro-N-methylbenzamide;
(R)-N-(8,9-Difluoro-6-oxo-1,4,5,6-tetrahydro-2H-pyrano[3,4-c]isoquinolin-1-yl)-4-(difluoromethyl)-3-fluoro-N-methylbenzamide;
(S)-N-(8,9-Difluoro-6-oxo-1,4,5,6-tetrahydro-2H-pyrano[3,4-c]isoquinolin-1-yl)-4-(difluoromethyl)-3-fluoro-N-methylbenzamide;
(R)-N-(8,9-Difluoro-6-oxo-1,4,5,6-tetrahydro-2H-pyrano[3,4-c]isoquinolin-1-yl)-4-(difluoromethyl)-N-methylbenzamide;
(S)-N-(8,9-Difluoro-6-oxo-1,4,5,6-tetrahydro-2H-pyrano[3,4-c]isoquinolin-1-yl)-4-(difluoromethyl)-N-methylbenzamide;
(R)-3-Chloro-N-(8,9-difluoro-6-oxo-1,4,5,6-tetrahydro-2H-pyrano[3,4-c]isoquinolin-1-yl)-4-fluoro-N-methylbenzamide;
(S)-3-Chloro-N-(8,9-difluoro-6-oxo-1,4,5,6-tetrahydro-2H-pyrano[3,4-c]isoquinolin-1-yl)-4-fluoro-N-methylbenzamide;
(R)-N-(8,9-Difluoro-6-oxo-1,2,3,4,5,6-hexahydrobenzo[c][1,7]naphthyridin-1-yl)-7-fluoro-N-methyl-1H-indole-2-carboxamide;
(S)-N-(8,9-Difluoro-6-oxo-1,2,3,4,5,6-hexahydrobenzo[c][1,7]naphthyridin-1-yl)-7-fluoro-N-methyl-1H-indole-2-carboxamide;
(R)-N-(8,9-Difluoro-6-oxo-1,2,3,4,5,6-hexahydrobenzo[c][1,7]naphthyridin-1-yl)-5,6-difluoro-N-methyl-1H-indole-2-carboxamide;
(S)-N-(8,9-Difluoro-6-oxo-1,2,3,4,5,6-hexahydrobenzo[c][1,7]naphthyridin-1-yl)-5,6-difluoro-N-methyl-1H-indole-2-carboxamide;
(R)-N-(8,9-Difluoro-6-oxo-1,2,3,4,5,6-hexahydrobenzo[c][1,7]naphthyridin-1-yl)-4,6-difluoro-N-methyl-1H-indole-2-carboxamide;
(S)-N-(8,9-Difluoro-6-oxo-1,2,3,4,5,6-hexahydrobenzo[c][1,7]naphthyridin-1-yl)-4,6-difluoro-N-methyl-1H-indole-2-carboxamide;
(R)-N-(8,9-Difluoro-6-oxo-1,2,3,4,5,6-hexahydrobenzo[c][1,7]naphthyridin-1-yl)-4,5-difluoro-N-methyl-1H-indole-2-carboxamide;
(S)-N-(8,9-Difluoro-6-oxo-1,2,3,4,5,6-hexahydrobenzo[c][1,7]naphthyridin-1-yl)-4,5-difluoro-N-methyl-1H-indole-2-carboxamide;
(R)-6-Chloro-N-(8,9-difluoro-6-oxo-1,2,3,4,5,6-hexahydrobenzo[c][1,7]naphthyridin-1-yl)-N-methylindolizine-2-carboxamide;
(S)-6-Chloro-N-(8,9-difluoro-6-oxo-1,2,3,4,5,6-hexahydrobenzo[c][1,7]naphthyridin-1-yl)-N-methylindolizine-2-carboxamide;
(R)-N-(8,9-Difluoro-6-oxo-1,4,5,6-tetrahydro-2H-pyrano[3,4-c]isoquinolin-1-yl)-N-methylbenzo[d]thiazole-2-carboxamide;
(S)-N-(8,9-Difluoro-6-oxo-1,4,5,6-tetrahydro-2H-pyrano[3,4-c]isoquinolin-1-yl)-N-methylbenzo[d]thiazole-2-carboxamide;
(R)-N-(8,9-Difluoro-6-oxo-1,4,5,6-tetrahydro-2H-pyrano[3,4-c]isoquinolin-1-yl)-N-methylbenzo[d]oxazole-2-carboxamide;
(S)-N-(8,9-Difluoro-6-oxo-1,4,5,6-tetrahydro-2H-pyrano[3,4-c]isoquinolin-1-yl)-N-methylbenzo[d]oxazole-2-carboxamide;
(R)-N-(8,9-Difluoro-6-oxo-1,4,5,6-tetrahydro-2H-pyrano[3,4-c]isoquinolin-1-yl)-N-methyl-3-(trifluoromethyl)-1H-pyrazole-5-carboxamide;
(S)-N-(8,9-Difluoro-6-oxo-1,4,5,6-tetrahydro-2H-pyrano[3,4-c]isoquinolin-1-yl)-N-methyl-3-(trifluoromethyl)-1H-pyrazole-5-carboxamide;
(R)-N-(8,9-Difluoro-6-oxo-1,4,5,6-tetrahydro-2H-pyrano[3,4-c]isoquinolin-1-yl)-3-(4-fluorophenyl)-N-methyl-1H-pyrazole-5-carboxamide;
(S)-N-(8,9-Difluoro-6-oxo-1,4,5,6-tetrahydro-2H-pyrano[3,4-c]isoquinolin-1-yl)-3-(4-fluorophenyl)-N-methyl-1H-pyrazole-5-carboxamide;
(R)-N-(8,9-Difluoro-6-oxo-1,4,5,6-tetrahydro-2H-pyrano[3,4-c]isoquinolin-1-yl)-3-fluoro-N-methylbenzamide;
(S)-N-(8,9-Difluoro-6-oxo-1,4,5,6-tetrahydro-2H-pyrano[3,4-c]isoquinolin-1-yl)-3-fluoro-N-methylbenzamide;
(R)-3-Chloro-N-(8,9-difluoro-6-oxo-1,4,5,6-tetrahydro-2H-pyrano[3,4-c]isoquinolin-1-yl)-N-methylbenzamide;
(S)-3-Chloro-N-(8,9-difluoro-6-oxo-1,4,5,6-tetrahydro-2H-pyrano[3,4-c]isoquinolin-1-yl)-N-methylbenzamide;
(R)-3-Bromo-N-(8,9-difluoro-6-oxo-1,4,5,6-tetrahydro-2H-pyrano[3,4-c]isoquinolin-1-yl)-N-methylbenzamide;
(S)-3-Bromo-N-(8,9-difluoro-6-oxo-1,4,5,6-tetrahydro-2H-pyrano[3,4-c]isoquinolin-1-yl)-N-methylbenzamide;
(R)-3-Bromo-N-(8,9-difluoro-6-oxo-1,4,5,6-tetrahydro-2H-pyrano[3,4-c]isoquinolin-1-yl)-4-fluoro-N-methylbenzamide;
(S)-3-Bromo-N-(8,9-difluoro-6-oxo-1,4,5,6-tetrahydro-2H-pyrano[3,4-c]isoquinolin-1-yl)-4-fluoro-N-methylbenzamide;
(R)-4-Bromo-N-(8,9-difluoro-6-oxo-1,4,5,6-tetrahydro-2H-pyrano[3,4-c]isoquinolin-1-yl)-N-methylbenzamide;
(S)-4-Bromo-N-(8,9-difluoro-6-oxo-1,4,5,6-tetrahydro-2H-pyrano[3,4-c]isoquinolin-1-yl)-N-methylbenzamide;
(R)-3-Fluoro-N-methyl-N-(6-oxo-1,4,5,6-tetrahydro-2H-pyrano[3,4-c]isoquinolin-1-yl)-4-(trifluoromethyl)benzamide;
(S)-3-Fluoro-N-methyl-N-(6-oxo-1,4,5,6-tetrahydro-2H-pyrano[3,4-c]isoquinolin-1-yl)-4-(trifluoromethyl)benzamide;
(R)-3-Fluoro-N-methyl-N-(4-oxo-4,5,8,9-tetrahydro-6H-pyrano[3,4-b]thieno[3,4-d]pyridin-9-yl)-4-(trifluoromethyl)benzamide;
(S)-3-Fluoro-N-methyl-N-(4-oxo-4,5,8,9-tetrahydro-6H-pyrano[3,4-b]thieno[3,4-d]pyridin-9-yl)-4-(trifluoromethyl)benzamide;
(R)-N-(8-Cyano-6-oxo-1,4,5,6-tetrahydro-2H-pyrano[3,4-c]isoquinolin-1-yl)-3-fluoro-N-methyl-4-(trifluoromethyl)benzamide;
(S)-N-(8-Cyano-6-oxo-1,4,5,6-tetrahydro-2H-pyrano[3,4-c]isoquinolin-1-yl)-3-fluoro-N-methyl-4-(trifluoromethyl)benzamide;
(R)-3-Cyano-N-(8,9-difluoro-6-oxo-1,4,5,6-tetrahydro-2H-pyrano[3,4-c]isoquinolin-1-yl)-N-methylbenzamide;
(S)-3-Cyano-N-(8,9-difluoro-6-oxo-1,4,5,6-tetrahydro-2H-pyrano[3,4-c]isoquinolin-1-yl)-N-methylbenzamide;
(R)-N-(8,9-Difluoro-6-oxo-1,4,5,6-tetrahydro-2H-pyrano[3,4-c]isoquinolin-1-yl)-3-fluoro-N-methyl-4-(trifluoromethoxy)benzamide;
(S)-N-(8,9-Difluoro-6-oxo-1,4,5,6-tetrahydro-2H-pyrano[3,4-c]isoquinolin-1-yl)-3-fluoro-N-methyl-4-(trifluoromethoxy)benzamide;
(R)-N-(8,9-Difluoro-6-oxo-1,4,5,6-tetrahydro-2H-pyrano[3,4-c]isoquinolin-1-yl)-3-fluoro-N,4-dimethylbenzamide;
(S)-N-(8,9-Difluoro-6-oxo-1,4,5,6-tetrahydro-2H-pyrano[3,4-c]isoquinolin-1-yl)-3-fluoro-N,4-dimethylbenzamide;
2-(3-Chlorophenyl)-N-((S)-8,9-difluoro-6-oxo-1,4,5,6-tetrahydro-2H-pyrano[3,4-c]isoquinolin-1-yl)-(2R)-hydroxy-N-methylacetamide;
2-(3-Chlorophenyl)-N-((S)-8,9-difluoro-6-oxo-1,4,5,6-tetrahydro-2H-pyrano[3,4-c]isoquinolin-1-yl)-(2S)-hydroxy-N-methylacetamide;
(R)-N-(8,9-Difluoro-6-oxo-1,4,5,6-tetrahydro-2H-pyrano[3,4-c]isoquinolin-1-yl)-3,5-difluoro-N-methyl-4-(trifluoromethyl)benzamide;
(S)-N-(8,9-Difluoro-6-oxo-1,4,5,6-tetrahydro-2H-pyrano[3,4-c]isoquinolin-1-yl)-3,5-difluoro-N-methyl-4-(trifluoromethyl)benzamide;
(R)-5-Chloro-N-(8,9-difluoro-6-oxo-1,4,5,6-tetrahydro-2H-pyrano[3,4-c]isoquinolin-1-yl)-N-methylthiophene-3-carboxamide;
(S)-5-Chloro-N-(8,9-difluoro-6-oxo-1,4,5,6-tetrahydro-2H-pyrano[3,4-c]isoquinolin-1-yl)-N-methylthiophene-3-carboxamide;
(2R)-Amino-N-((R)-8,9-difluoro-6-oxo-1,4,5,6-tetrahydro-2H-pyrano[3,4-c]isoquinolin-1-yl)-(3R)-hydroxy-N-methylbutanamide;
(2R)-Amino-N-((R)-8,9-difluoro-6-oxo-1,4,5,6-tetrahydro-2H-pyrano[3,4-c]isoquinolin-1-yl)-(3S)-hydroxy-N-methylbutanamide;
(2S)-Amino-N-((R)-8,9-difluoro-6-oxo-1,4,5,6-tetrahydro-2H-pyrano[3,4-c]isoquinolin-1-yl)-(3R)-hydroxy-N-methylbutanamide;
(2S)-Amino-N-((R)-8,9-difluoro-6-oxo-1,4,5,6-tetrahydro-2H-pyrano[3,4-c]isoquinolin-1-yl)-(3S)-hydroxy-N-methylbutanamide;
(2R)-Amino-N-((S)-8,9-difluoro-6-oxo-1,4,5,6-tetrahydro-2H-pyrano[3,4-c]isoquinolin-1-yl)-(3R)-hydroxy-N-methylbutanamide;
(2R)-Amino-N-((S)-8,9-difluoro-6-oxo-1,4,5,6-tetrahydro-2H-pyrano[3,4-c]isoquinolin-1-yl)-(3S)-hydroxy-N-methylbutanamide;
(2S)-Amino-N-((S)-8,9-difluoro-6-oxo-1,4,5,6-tetrahydro-2H-pyrano[3,4-c]isoquinolin-1-yl)-(3R)-hydroxy-N-methylbutanamide;
(2S)-Amino-N-((S)-8,9-difluoro-6-oxo-1,4,5,6-tetrahydro-2H-pyrano[3,4-c]isoquinolin-1-yl)-(3S)-hydroxy-N-methylbutanamide;
(R)-3-Chloro-N-(8,9-difluoro-6-oxo-1,4,5,6-tetrahydro-2H-pyrano[3,4-c]isoquinolin-1-yl)-4-fluoro-N-methylbenzenesulfonamide;
(S)-3-Chloro-N-(8,9-difluoro-6-oxo-1,4,5,6-tetrahydro-2H-pyrano[3,4-c]isoquinolin-1-yl)-4-fluoro-N-methylbenzenesulfonamide;
(R)-3-Chloro-N-(8,9-difluoro-6-oxo-1,4,5,6-tetrahydro-2H-pyrano[3,4-c]isoquinolin-1-yl)-N-methylbenzenesulfonamide;
(S)-3-Chloro-N-(8,9-difluoro-6-oxo-1,4,5,6-tetrahydro-2H-pyrano[3,4-c]isoquinolin-1-yl)-N-methylbenzenesulfonamide;
(R)-3-Cyano-N-(8,9-difluoro-6-oxo-1,4,5,6-tetrahydro-2H-pyrano[3,4-c]isoquinolin-1-yl)-N-methylbenzenesulfonamide;
(S)-3-Cyano-N-(8,9-difluoro-6-oxo-1,4,5,6-tetrahydro-2H-pyrano[3,4-c]isoquinolin-1-yl)-N-methylbenzenesulfonamide;
(R)-3-Cyano-N-(8,9-difluoro-6-oxo-1,4,5,6-tetrahydro-2H-pyrano[3,4-c]isoquinolin-1-yl)-4-fluoro-N-methylbenzenesulfonamide;
(S)-3-Cyano-N-(8,9-difluoro-6-oxo-1,4,5,6-tetrahydro-2H-pyrano[3,4-c]isoquinolin-1-yl)-4-fluoro-N-methylbenzenesulfonamide;
(R)-N-(8,9-Difluoro-6-oxo-1,4,5,6-tetrahydro-2H-pyrano[3,4-c]isoquinolin-1-yl)-4-ethyl-3-fluoro-N-methylbenzamide;
(S)-N-(8,9-Difluoro-6-oxo-1,4,5,6-tetrahydro-2H-pyrano[3,4-c]isoquinolin-1-yl)-4-ethyl-3-fluoro-N-methylbenzamide;
(S)-N-(8,9-difluoro-6-oxo-1,4,5,6-tetrahydro-2H-pyrano[3,4-c]isoquinolin-1-yl)indolizine-2-carboxamide;
(R)-N-(8,9-difluoro-6-oxo-1,4,5,6-tetrahydro-2H-pyrano[3,4-c]isoquinolin-1-yl)indolizine-2-carboxamide;
(R,R)-N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
(R,S)-N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
(S,R)-N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
(S,S)-N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
(S)-N-(8,9-difluoro-3,3-dioxido-6-oxo-1,4,5,6-tetrahydro-2H-thiopyrano[3,4-c]isoquinolin-1-yl)-8-fluoro-N-methylindolizine-2-carboxamide;
(R)-N-(8,9-difluoro-3,3-dioxido-6-oxo-1,4,5,6-tetrahydro-2H-thiopyrano[3,4-c]isoquinolin-1-yl)-8-fluoro-N-methylindolizine-2-carboxamide;
(S)-N-(8,9-difluoro-6-oxo-1,4,5,6-tetrahydro-2H-pyrano[3,4-c]isoquinolin-1-yl)-4-(difluoromethoxy)-3-fluoro-N-methylbenzamide;
(R)-N-(8,9-difluoro-6-oxo-1,4,5,6-tetrahydro-2H-pyrano[3,4-c]isoquinolin-1-yl)-4-(difluoromethoxy)-3-fluoro-N-methylbenzamide;
(S)-N-(8,9-difluoro-6-oxo-1,4,5,6-tetrahydro-2H-pyrano[3,4-c]isoquinolin-1-yl)-4-(2-hydroxypropan-2-yl)-N-methylbenzamide;
(R)-N-(8,9-difluoro-6-oxo-1,4,5,6-tetrahydro-2H-pyrano[3,4-c]isoquinolin-1-yl)-4-(2-hydroxypropan-2-yl)-N-methylbenzamide;
(S)-N-(8,9-difluoro-6-oxo-1,4,5,6-tetrahydro-2H-pyrano[3,4-c]isoquinolin-1-yl)-3-(2-hydroxypropan-2-yl)-N-methylbenzamide;
(R)-N-(8,9-difluoro-6-oxo-1,4,5,6-tetrahydro-2H-pyrano[3,4-c]isoquinolin-1-yl)-3-(2-hydroxypropan-2-yl)-N-methylbenzamide;
(S)-N-(8,9-difluoro-6-oxo-1,4,5,6-tetrahydro-2H-pyrano[3,4-c]isoquinolin-1-yl)-5,6-difluoro-1H-indole-2-carboxamide;
(R)-N-(8,9-difluoro-6-oxo-1,4,5,6-tetrahydro-2H-pyrano[3,4-c]isoquinolin-1-yl)-5,6-difluoro-1H-indole-2-carboxamide;
(S)-N-(8,9-difluoro-6-oxo-1,4,5,6-tetrahydro-2H-pyrano[3,4-c]isoquinolin-1-yl)-4,6-difluoro-1H-indole-2-carboxamide;
(R)-N-(8,9-difluoro-6-oxo-1,4,5,6-tetrahydro-2H-pyrano[3,4-c]isoquinolin-1-yl)-4,6-difluoro-1H-indole-2-carboxamide;
(S)-N-(8,9-difluoro-6-oxo-1,4,5,6-tetrahydro-2H-pyrano[3,4-c]isoquinolin-1-yl)-3-fluoro-4-(trifluoromethyl)benzamide;
(R)-N-(8,9-difluoro-6-oxo-1,4,5,6-tetrahydro-2H-pyrano[3,4-c]isoquinolin-1-yl)-3-fluoro-4-(trifluoromethyl)benzamide;
(S)-4-chloro-N-(8,9-difluoro-6-oxo-1,4,5,6-tetrahydro-2H-pyrano[3,4-c]isoquinolin-1-yl)-3-fluorobenzamide;
(R)-4-chloro-N-(8,9-difluoro-6-oxo-1,4,5,6-tetrahydro-2H-pyrano[3,4-c]isoquinolin-1-yl)-3-fluorobenzamide;
(S)-N-(8,9-difluoro-6-oxo-1,2,3,4,5,6-hexahydrobenzo[c][1,7]naphthyridin-1-yl)-8-fluoro-N-methylindolizine-2-carboxamide;
(R)-N-(8,9-difluoro-6-oxo-1,2,3,4,5,6-hexahydrobenzo[c][1,7]naphthyridin-1-yl)-8-fluoro-N-methylindolizine-2-carboxamide;
(S)-N-(8,9-difluoro-6-oxo-1,2,3,4,5,6-hexahydrobenzo[c][1,7]naphthyridin-1-yl)-7-fluoro-N-methylindolizine-2-carboxamide;
(R)-N-(8,9-difluoro-6-oxo-1,2,3,4,5,6-hexahydrobenzo[c][1,7]naphthyridin-1-yl)-7-fluoro-N-methylindolizine-2-carboxamide;
(S)-4-bromo-N-(8,9-difluoro-6-oxo-1,2,3,4,5,6-hexahydrobenzo[c][1,7]naphthyridin-1-yl)-3,5-difluoro-N-methylbenzamide;
(R)-4-bromo-N-(8,9-difluoro-6-oxo-1,2,3,4,5,6-hexahydrobenzo[c][1,7]naphthyridin-1-yl)-3,5-difluoro-N-methylbenzamide;
(S)-4-bromo-3,5-difluoro-N-(8-fluoro-6-oxo-1,4,5,6-tetrahydro-2H-pyrano[3,4-c]isoquinolin-1-yl)-N-methylbenzamide;
(R)-4-bromo-3,5-difluoro-N-(8-fluoro-6-oxo-1,4,5,6-tetrahydro-2H-pyrano[3,4-c]isoquinolin-1-yl)-N-methylbenzamide;
(S)-4-chloro-N-(8,9-difluoro-6-oxo-1,2,3,4,5,6-hexahydrobenzo[c][1,7]naphthyridin-1-yl)-3,5-difluoro-N-methylbenzamide;
(R)-4-chloro-N-(8,9-difluoro-6-oxo-1,2,3,4,5,6-hexahydrobenzo[c][1,7]naphthyridin-1-yl)-3,5-difluoro-N-methylbenzamide;
(S)-4-bromo-3,5-difluoro-N-(8-fluoro-6-oxo-1,2,3,4,5,6-hexahydrobenzo[c][1,7]naphthyridin-1-yl)-N-methylbenzamide;
(R)-4-bromo-3,5-difluoro-N-(8-fluoro-6-oxo-1,2,3,4,5,6-hexahydrobenzo[c][1,7]naphthyridin-1-yl)-N-methylbenzamide;
(S)-4-bromo-N-(8,9-difluoro-6-oxo-1,4,5,6-tetrahydro-2H-pyrano[3,4-c]isoquinolin-1-yl)-3,5-difluoro-N-methylbenzamide;
(R)-4-bromo-N-(8,9-difluoro-6-oxo-1,4,5,6-tetrahydro-2H-pyrano[3,4-c]isoquinolin-1-yl)-3,5-difluoro-N-methylbenzamide;
(S)-4-chloro-N-(8,9-difluoro-6-oxo-1,4,5,6-tetrahydro-2H-pyrano[3,4-c]isoquinolin-1-yl)-3,5-difluoro-N-methylbenzamide;
(R)-4-chloro-N-(8,9-difluoro-6-oxo-1,4,5,6-tetrahydro-2H-pyrano[3,4-c]isoquinolin-1-yl)-3,5-difluoro-N-methylbenzamide;
(S)-4-chloro-3,5-difluoro-N-(8-fluoro-6-oxo-1,4,5,6-tetrahydro-2H-pyrano[3,4-c]isoquinolin-1-yl)-N-methylbenzamide;
(R)-4-chloro-3,5-difluoro-N-(8-fluoro-6-oxo-1,4,5,6-tetrahydro-2H-pyrano[3,4-c]isoquinolin-1-yl)-N-methylbenzamide;
(S)-4-chloro-3,5-difluoro-N-(8-fluoro-6-oxo-1,2,3,4,5,6-hexahydrobenzo[c][1,7]naphthyridin-1-yl)-N-methylbenzamide;
(R)-4-chloro-3,5-difluoro-N-(8-fluoro-6-oxo-1,2,3,4,5,6-hexahydrobenzo[c][1,7]naphthyridin-1-yl)-N-methylbenzamide;
(S)-4-(difluoromethyl)-3,5-difluoro-N-(8-fluoro-6-oxo-1,4,5,6-tetrahydro-2H-pyrano[3,4-c]isoquinolin-1-yl)-N-methylbenzamide;
(R)-4-(difluoromethyl)-3,5-difluoro-N-(8-fluoro-6-oxo-1,4,5,6-tetrahydro-2H-pyrano[3,4-c]isoquinolin-1-yl)-N-methylbenzamide;
(S)-N-(8,9-difluoro-6-oxo-1,4,5,6-tetrahydro-2H-pyrano[3,4-c]isoquinolin-1-yl)-4-(difluoromethyl)-3,5-difluoro-N-methylbenzamide;
(R)-N-(8,9-difluoro-6-oxo-1,4,5,6-tetrahydro-2H-pyrano[3,4-c]isoquinolin-1-yl)-4-(difluoromethyl)-3,5-difluoro-N-methylbenzamide;
(S)-4-(difluoromethyl)-N-(8-fluoro-6-oxo-1,4,5,6-tetrahydro-2H-pyrano[3,4-c]isoquinolin-1-yl)-N-methyl-1H-indole-2-carboxamide;
(R)-4-(difluoromethyl)-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,2,3,4,5,6-hexahydrobenzo[c][1,7]naphthyridin-1-yl)-4-(difluoromethyl)-3,5-difluoro-N-methylbenzamide;
(R)-N-(8,9-difluoro-6-oxo-1,2,3,4,5,6-hexahydrobenzo[c][1,7]naphthyridin-1-yl)-4-(difluoromethyl)-3,5-difluoro-N-methylbenzamide;
(S)-4-(difluoromethyl)-3,5-difluoro-N-(8-fluoro-6-oxo-1,2,3,4,5,6-hexahydrobenzo[c][1,7]naphthyridin-1-yl)-N-methylbenzamide;
(R)-4-(difluoromethyl)-3,5-difluoro-N-(8-fluoro-6-oxo-1,2,3,4,5,6-hexahydrobenzo[c][1,7]naphthyridin-1-yl)-N-methylbenzamide;
N-((1S)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1S,4R)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1S,4S)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1R)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1R,4R)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1R,4S)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-N,1-dimethyl-1H-pyrazole-4-carboxamide;
(R)-N-(8,9-difluoro-6-oxo-1,4,5,6-tetrahydro-2H-pyrano[3,4-c]isoquinolin-1-yl)-N,1-dimethyl-1H-pyrazole-4-carboxamide;
(S)-N-(8,9-difluoro-6-oxo-1,4,5,6-tetrahydro-2H-pyrano[3,4-c]isoquinolin-1-yl)-N-methyl-2-phenylacrylamide;
(R)-N-(8,9-difluoro-6-oxo-1,4,5,6-tetrahydro-2H-pyrano[3,4-c]isoquinolin-1-yl)-N-methyl-2-phenylacrylamide;
(S)-N-(8,9-difluoro-6-oxo-1,4,5,6-tetrahydro-2H-pyrano[3,4-c]isoquinolin-1-yl)-5-fluoro-N-methyl-1H-pyrrolo[2,3-b]pyridine-2-carboxamide;
(R)-N-(8,9-difluoro-6-oxo-1,4,5,6-tetrahydro-2H-pyrano[3,4-c]isoquinolin-1-yl)-5-fluoro-N-methyl-1H-pyrrolo[2,3-b]pyridine-2-carboxamide;
(S)-5-chloro-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
(R)-5-chloro-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
(S)-5-bromo-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
(R)-5-bromo-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
(S)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-3-carboxamide;
(R)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-3-carboxamide;
(S)-5-bromo-N-(8,9-difluoro-6-oxo-1,4,5,6-tetrahydro-2H-pyrano[3,4-c]isoquinolin-1-yl)-N-methylthiophene-3-carboxamide;
(R)-5-bromo-N-(8,9-difluoro-6-oxo-1,4,5,6-tetrahydro-2H-pyrano[3,4-c]isoquinolin-1-yl)-N-methylthiophene-3-carboxamide;
(S)-N-(8,9-difluoro-6-oxo-1,4,5,6-tetrahydro-2H-pyrano[3,4-c]isoquinolin-1-yl)-N-methyl-2,3-dihydro-1H-indene-2-carboxamide;
(R)-N-(8,9-difluoro-6-oxo-1,4,5,6-tetrahydro-2H-pyrano[3,4-c]isoquinolin-1-yl)-N-methyl-2,3-dihydro-1H-indene-2-carboxamide;
(S)-1-(tert-butyl)-N-(8,9-difluoro-6-oxo-1,4,5,6-tetrahydro-2H-pyrano[3,4-c]isoquinolin-1-yl)-N-methyl-1H-pyrazole-4-carboxamide;
(R)-1-(tert-butyl)-N-(8,9-difluoro-6-oxo-1,4,5,6-tetrahydro-2H-pyrano[3,4-c]isoquinolin-1-yl)-N-methyl-1H-pyrazole-4-carboxamide;
(S)-N-(8,9-difluoro-6-oxo-1,4,5,6-tetrahydro-2H-pyrano[3,4-c]isoquinolin-1-yl)-N-methyl-1-(trifluoromethyl)-1H-pyrazole-4-carboxamide;
(R)-N-(8,9-difluoro-6-oxo-1,4,5,6-tetrahydro-2H-pyrano[3,4-c]isoquinolin-1-yl)-N-methyl-1-(trifluoromethyl)-1H-pyrazole-4-carboxamide;
(S)-4-bromo-N-(8,9-difluoro-6-oxo-1,4,5,6-tetrahydro-2H-pyrano[3,4-c]isoquinolin-1-yl)-N-methyl-1H-pyrrole-2-carboxamide;
(R)-4-bromo-N-(8,9-difluoro-6-oxo-1,4,5,6-tetrahydro-2H-pyrano[3,4-c]isoquinolin-1-yl)-N-methyl-1H-pyrrole-2-carboxamide;
(S)-2-amino-2-(4-chlorophenyl)-N-((S)-8,9-difluoro-6-oxo-1,4,5,6-tetrahydro-2H-pyrano[3,4-c]isoquinolin-1-yl)-N-methylacetamide;
(R)-2-amino-2-(4-chlorophenyl)-N-((S)-8,9-difluoro-6-oxo-1,4,5,6-tetrahydro-2H-pyrano[3,4-c]isoquinolin-1-yl)-N-methylacetamide;
(S)-2-amino-2-(4-chlorophenyl)-N-((R)-8,9-difluoro-6-oxo-1,4,5,6-tetrahydro-2H-pyrano[3,4-c]isoquinolin-1-yl)-N-methylacetamide;
(R)-2-amino-2-(4-chlorophenyl)-N-((R)-8,9-difluoro-6-oxo-1,4,5,6-tetrahydro-2H-pyrano[3,4-c]isoquinolin-1-yl)-N-methylacetamide;
2-amino-2-(4-chlorophenyl)-N-((S)-8,9-difluoro-6-oxo-1,4,5,6-tetrahydro-2H-pyrano[3,4-c]isoquinolin-1-yl)-N-methylacetamide;
2-amino-2-(4-chlorophenyl)-N-((S)-8,9-difluoro-6-oxo-1,4,5,6-tetrahydro-2H-pyrano[3,4-c]isoquinolin-1-yl)-N-methylacetamide;
(S)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
(R)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
(S)-2-amino-2-(3-chlorophenyl)-N-((S)-8,9-difluoro-6-oxo-1,4,5,6-tetrahydro-2H-pyrano[3,4-c]isoquinolin-1-yl)-N-methylacetamide;
(R)-2-amino-2-(3-chlorophenyl)-N-((S)-8,9-difluoro-6-oxo-1,4,5,6-tetrahydro-2H-pyrano[3,4-c]isoquinolin-1-yl)-N-methylacetamide;
(S)-2-amino-2-(3-chlorophenyl)-N-((R)-8,9-difluoro-6-oxo-1,4,5,6-tetrahydro-2H-pyrano[3,4-c]isoquinolin-1-yl)-N-methylacetamide;
(R)-2-amino-2-(3-chlorophenyl)-N-((R)-8,9-difluoro-6-oxo-1,4,5,6-tetrahydro-2H-pyrano[3,4-c]isoquinolin-1-yl)-N-methylacetamide;
2-amino-2-(3-chlorophenyl)-N-((S)-8,9-difluoro-6-oxo-1,4,5,6-tetrahydro-2H-pyrano[3,4-c]isoquinolin-1-yl)-N-methylacetamide;
2-amino-2-(3-chlorophenyl)-N-((R)-8,9-difluoro-6-oxo-1,4,5,6-tetrahydro-2H-pyrano[3,4-c]isoquinolin-1-yl)-N-methylacetamide;
(S)-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
(R)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
(S)-5-chloro-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
(R)-5-chloro-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
(S)-N-(8,9-difluoro-6-oxo-1,2,3,4,5,6-hexahydrobenzo[c][1,7]naphthyridin-1-yl)-4-(difluoromethyl)-N-methyl-1H-indole-2-carboxamide;
(R)-N-(8,9-difluoro-6-oxo-1,2,3,4,5,6-hexahydrobenzo[c][1,7]naphthyridin-1-yl)-4-(difluoromethyl)-N-methyl-1H-indole-2-carboxamide;
(S)-4-(difluoromethyl)-N-(8-fluoro-6-oxo-1,2,3,4,5,6-hexahydrobenzo[c][1,7]naphthyridin-1-yl)-N-methyl-1H-indole-2-carboxamide;
(R)-4-(difluoromethyl)-N-(8-fluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-1-hydroxy-N-methylcyclohexane-1-carboxamide;
(R)-N-(8,9-difluoro-6-oxo-1,4,5,6-tetrahydro-2H-pyrano[3,4-c]isoquinolin-1-yl)-1-hydroxy-N-methylcyclohexane-1-carboxamide;
(S)-N-(8,9-difluoro-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(S)-5-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-5-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-b]pyridine-2-carboxamide;
(R)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-b]pyridine-2-carboxamide;
(S)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
(R)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
(S)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
(R)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
(S)-6-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
(R)-6-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[3,2-b]pyridine-2-carboxamide;
(R)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[3,2-b]pyridine-2-carboxamide;
(S)-N-(8,9-difluoro-6-oxo-1,2,3,4,5,6-hexahydrobenzo[c][1,7]naphthyridin-1-yl)-6-(difluoromethyl)-5-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-6-(difluoromethyl)-5-fluoro-N-methyl-1H-indole-2-carboxamide;
(S)-N-(8,9-difluoro-6-oxo-1,2,3,4,5,6-hexahydrobenzo[c][1,7]naphthyridin-1-yl)-6-fluoro-N-methylindolizine-2-carboxamide;
(R)-N-(8,9-difluoro-6-oxo-1,2,3,4,5,6-hexahydrobenzo[c][1,7]naphthyridin-1-yl)-6-fluoro-N-methylindolizine-2-carboxamide;
(S)-N-(8,9-difluoro-6-oxo-1,4,5,6-tetrahydro-2H-pyrano[3,4-c]isoquinolin-1-yl)-6-fluoro-N-methylindolizine-2-carboxamide;
(R)-N-(8,9-difluoro-6-oxo-1,4,5,6-tetrahydro-2H-pyrano[3,4-c]isoquinolin-1-yl)-6-fluoro-N-methylindolizine-2-carboxamide;
(S)-N-(8,9-difluoro-6-oxo-1,4,5,6-tetrahydro-2H-pyrano[3,4-c]isoquinolin-1-yl)-N-methyl-2-(trifluoromethyl)-1H-imidazole-5-carboxamide;
(R)-N-(8,9-difluoro-6-oxo-1,4,5,6-tetrahydro-2H-pyrano[3,4-c]isoquinolin-1-yl)-N-methyl-2-(trifluoromethyl)-1H-imidazole-5-carboxamide;
(S)-N-(8,9-difluoro-6-oxo-1,4,5,6-tetrahydro-2H-pyrano[3,4-c]isoquinolin-1-yl)-5-fluoro-N-methyl-6-oxo-1,6-dihydropyridine-3-carboxamide;
(R)-N-(8,9-difluoro-6-oxo-1,4,5,6-tetrahydro-2H-pyrano[3,4-c]isoquinolin-1-yl)-5-fluoro-N-methyl-6-oxo-1,6-dihydropyridine-3-carboxamide;
(S)-N-(8,9-difluoro-6-oxo-1,4,5,6-tetrahydro-2H-pyrano[3,4-c]isoquinolin-1-yl)-N-methylindolizine-6-carboxamide;
(R)-N-(8,9-difluoro-6-oxo-1,4,5,6-tetrahydro-2H-pyrano[3,4-c]isoquinolin-1-yl)-N-methylindolizine-6-carboxamide;
(S)-N-(8,9-difluoro-6-oxo-1,4,5,6-tetrahydro-2H-pyrano[3,4-c]isoquinolin-1-yl)-N-methylindolizine-7-carboxamide;
(R)-N-(8,9-difluoro-6-oxo-1,4,5,6-tetrahydro-2H-pyrano[3,4-c]isoquinolin-1-yl)-N-methylindolizine-7-carboxamide;
(S)-N-(8,9-difluoro-6-oxo-1,4,5,6-tetrahydro-2H-pyrano[3,4-c]isoquinolin-1-yl)-2-hydroxy-N-methyl-2,2-diphenylacetamide;
(R)-N-(8,9-difluoro-6-oxo-1,4,5,6-tetrahydro-2H-pyrano[3,4-c]isoquinolin-1-yl)-2-hydroxy-N-methyl-2,2-diphenylacetamide;
(S)-N-(8,9-difluoro-6-oxo-1,4,5,6-tetrahydro-2H-pyrano[3,4-c]isoquinolin-1-yl)-4-(difluoromethyl)-6-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4-(difluoromethyl)-6-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-7-fluoro-N-methylindolizine-2-carboxamide;
(R)-N-(8,9-difluoro-6-oxo-1,4,5,6-tetrahydro-2H-pyrano[3,4-c]isoquinolin-1-yl)-7-fluoro-N-methylindolizine-2-carboxamide;
(S)-N-(8,9-difluoro-6-oxo-1,4,5,6-tetrahydro-2H-pyrano[3,4-c]isoquinolin-1-yl)-5-fluoro-N-methyl-1H-pyrrolo[3,2-b]pyridine-2-carboxamide;
(R)-N-(8,9-difluoro-6-oxo-1,4,5,6-tetrahydro-2H-pyrano[3,4-c]isoquinolin-1-yl)-5-fluoro-N-methyl-1H-pyrrolo[3,2-b]pyridine-2-carboxamide;
(S)-N-(8,9-difluoro-6-oxo-1,4,5,6-tetrahydro-2H-pyrano[3,4-c]isoquinolin-1-yl)-5,6-difluoro-N-methyl-2,3-dihydro-1H-indene-2-carboxamide;
(R)-N-(8,9-difluoro-6-oxo-1,4,5,6-tetrahydro-2H-pyrano[3,4-c]isoquinolin-1-yl)-5,6-difluoro-N-methyl-2,3-dihydro-1H-indene-2-carboxamide;
(S)-N-(8,9-difluoro-6-oxo-1,4,5,6-tetrahydro-2H-pyrano[3,4-c]isoquinolin-1-yl)-1-(4-fluorophenyl)-N-methyl-1H-pyrazole-4-carboxamide;
(R)-N-(8,9-difluoro-6-oxo-1,4,5,6-tetrahydro-2H-pyrano[3,4-c]isoquinolin-1-yl)-1-(4-fluorophenyl)-N-methyl-1H-pyrazole-4-carboxamide;
(S)-N-(8,9-difluoro-6-oxo-1,4,5,6-tetrahydro-2H-pyrano[3,4-c]isoquinolin-1-yl)-3-(4-fluorophenyl)-N-methylisoxazole-5-carboxamide;
(R)-N-(8,9-difluoro-6-oxo-1,4,5,6-tetrahydro-2H-pyrano[3,4-c]isoquinolin-1-yl)-3-(4-fluorophenyl)-N-methylisoxazole-5-carboxamide;
(S)-N-(8,9-difluoro-6-oxo-1,4,5,6-tetrahydro-2H-pyrano[3,4-c]isoquinolin-1-yl)-4-(3-fluorophenoxy)-N-methylbenzamide;
(R)-N-(8,9-difluoro-6-oxo-1,4,5,6-tetrahydro-2H-pyrano[3,4-c]isoquinolin-1-yl)-4-(3-fluorophenoxy)-N-methylbenzamide;
(S)-4-(difluoromethyl)-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-(difluoromethyl)-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-5-methyl-6-oxo-1,4,5,6-tetrahydro-2H-pyrano[3,4-c]isoquinolin-1-yl)-5,6-difluoro-N-methyl-1H-indole-2-carboxamide;
(R)-N-(8,9-difluoro-5-methyl-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-methoxy-1,4-dihydro-2H-pyrano[3,4-c]isoquinolin-1-yl)-5,6-difluoro-N-methyl-1H-indole-2-carboxamide;
(R)-N-(8,9-difluoro-6-methoxy-1,4-dihydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-5,6,7,8-tetrahydroindolizine-2-carboxamide;
(R)-N-(8,9-difluoro-6-oxo-1,4,5,6-tetrahydro-2H-pyrano[3,4-c]isoquinolin-1-yl)-N-methyl-5,6,7,8-tetrahydroindolizine-2-carboxamide;
(S)-4-bromo-N-(8,9-difluoro-6-oxo-1,4,5,6-tetrahydro-2H-pyrano[3,4-c]isoquinolin-1-yl)-2,3-difluoro-N-methylbenzamide;
(R)-4-bromo-N-(8,9-difluoro-6-oxo-1,4,5,6-tetrahydro-2H-pyrano[3,4-c]isoquinolin-1-yl)-2,3-difluoro-N-methylbenzamide;
(S)-4-bromo-N-(8,9-difluoro-6-oxo-1,4,5,6-tetrahydro-2H-pyrano[3,4-c]isoquinolin-1-yl)-2,5-difluoro-N-methylbenzamide;
(R)-4-bromo-N-(8,9-difluoro-6-oxo-1,4,5,6-tetrahydro-2H-pyrano[3,4-c]isoquinolin-1-yl)-2,5-difluoro-N-methylbenzamide;
(S)-4-bromo-N-(8,9-difluoro-6-oxo-1,4,5,6-tetrahydro-2H-pyrano[3,4-c]isoquinolin-1-yl)-2,6-difluoro-N-methylbenzamide;
(R)-4-bromo-N-(8,9-difluoro-6-oxo-1,4,5,6-tetrahydro-2H-pyrano[3,4-c]isoquinolin-1-yl)-2,6-difluoro-N-methylbenzamide;
(S)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-2-carboxamide;
(R)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-2-carboxamide;
(S)-N-(8,9-difluoro-6-oxo-1,4,5,6-tetrahydro-2H-pyrano[3,4-c]isoquinolin-1-yl)-N-methylindolizine-3-carboxamide;
(R)-N-(8,9-difluoro-6-oxo-1,4,5,6-tetrahydro-2H-pyrano[3,4-c]isoquinolin-1-yl)-N-methylindolizine-3-carboxamide;
(S)-N-(8,9-difluoro-6-oxo-1,4,5,6-tetrahydro-2H-pyrano[3,4-c]isoquinolin-1-yl)-4-(4-fluorophenoxy)-N-methylbenzamide;
(R)-N-(8,9-difluoro-6-oxo-1,4,5,6-tetrahydro-2H-pyrano[3,4-c]isoquinolin-1-yl)-4-(4-fluorophenoxy)-N-methylbenzamide;
(S)-N-(8,9-difluoro-6-oxo-1,4,5,6-tetrahydro-2H-pyrano[3,4-c]isoquinolin-1-yl)-4′-fluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-4′-fluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-c]pyridine-2-carboxamide;
(R)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-c]pyridine-2-carboxamide;
(S)-2-(3-bromophenyl)-N-(8,9-difluoro-6-oxo-1,4,5,6-tetrahydro-2H-pyrano[3,4-c]isoquinolin-1-yl)-2,2-difluoro-N-methylacetamide;
(R)-2-(3-bromophenyl)-N-(8,9-difluoro-6-oxo-1,4,5,6-tetrahydro-2H-pyrano[3,4-c]isoquinolin-1-yl)-2,2-difluoro-N-methylacetamide;
(S)-2-(4-bromophenyl)-N-(8,9-difluoro-6-oxo-1,4,5,6-tetrahydro-2H-pyrano[3,4-c]isoquinolin-1-yl)-2,2-difluoro-N-methylacetamide;
(R)-2-(4-bromophenyl)-N-(8,9-difluoro-6-oxo-1,4,5,6-tetrahydro-2H-pyrano[3,4-c]isoquinolin-1-yl)-2,2-difluoro-N-methylacetamide;
(S)-N-(8,9-difluoro-6-oxo-1,4,5,6-tetrahydro-2H-pyrano[3,4-c]isoquinolin-1-yl)-4-(difluoromethyl)-5-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4-(difluoromethyl)-5-fluoro-N-methyl-1H-indole-2-carboxamide;
(S)-4-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,2,3,4,5,6-hexahydrobenzo[c][1,7]naphthyridin-1-yl)-4-(difluoromethyl)-6-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-4-(difluoromethyl)-6-fluoro-N-methyl-1H-indole-2-carboxamide;
(S)-4-(difluoromethyl)-6-fluoro-N-(8-fluoro-6-oxo-1,2,3,4,5,6-hexahydrobenzo[c][1,7]naphthyridin-1-yl)-N-methyl-1H-indole-2-carboxamide;
(R)-4-(difluoromethyl)-6-fluoro-N-(8-fluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methylindolizine-1-carboxamide;
(R)-N-(8,9-difluoro-6-oxo-1,4,5,6-tetrahydro-2H-pyrano[3,4-c]isoquinolin-1-yl)-N-methylindolizine-1-carboxamide;
(S)-N-(8,9-difluoro-6-oxo-1,4,5,6-tetrahydro-2H-pyrano[3,4-c]isoquinolin-1-yl)-2,4′-difluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-2,4′-difluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-5-(4-fluorophenyl)-N-methylisoxazole-3-carboxamide;
(R)-N-(8,9-difluoro-6-oxo-1,4,5,6-tetrahydro-2H-pyrano[3,4-c]isoquinolin-1-yl)-5-(4-fluorophenyl)-N-methylisoxazole-3-carboxamide;
(S)-N-(8,9-difluoro-6-oxo-1,2,3,4,5,6-hexahydrobenzo[c][1,7]naphthyridin-1-yl)-4-(difluoromethyl)-5-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-4-(difluoromethyl)-5-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-5-(difluoromethyl)-N-methylindolizine-2-carboxamide;
(R)-N-(8,9-difluoro-6-oxo-1,4,5,6-tetrahydro-2H-pyrano[3,4-c]isoquinolin-1-yl)-5-(difluoromethyl)-N-methylindolizine-2-carboxamide;
(S)-5-bromo-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
(R)-5-bromo-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
N-((S)-8,9-difluoro-6-oxo-1,4,5,6-tetrahydro-2H-pyrano[3,4-c]isoquinolin-1-yl)-N-methylbicyclo[4.2.0]octa-1(6),2,4-triene-7-carboxamide;
N-((R)-8,9-difluoro-6-oxo-1,4,5,6-tetrahydro-2H-pyrano[3,4-c]isoquinolin-1-yl)-N-methylbicyclo[4.2.0]octa-1(6),2,4-triene-7-carboxamide;
(S)-4-(difluoromethyl)-5-fluoro-N-(8-fluoro-6-oxo-1,2,3,4,5,6-hexahydrobenzo[c][1,7]naphthyridin-1-yl)-N-methyl-1H-indole-2-carboxamide;
(R)-4-(difluoromethyl)-5-fluoro-N-(8-fluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-2-hydroxy-N-methyl-2,3-dihydro-1H-indene-2-carboxamide;
(R)-N-(8,9-difluoro-6-oxo-1,4,5,6-tetrahydro-2H-pyrano[3,4-c]isoquinolin-1-yl)-2-hydroxy-N-methyl-2,3-dihydro-1H-indene-2-carboxamide;
(S)-N-(8,9-difluoro-6-oxo-1,4,5,6-tetrahydro-2H-pyrano[3,4-c]isoquinolin-1-yl)-5,6-difluoro-2-hydroxy-N-methyl-2,3-dihydro-1H-indene-2-carboxamide;
(R)-N-(8,9-difluoro-6-oxo-1,4,5,6-tetrahydro-2H-pyrano[3,4-c]isoquinolin-1-yl)-5,6-difluoro-2-hydroxy-N-methyl-2,3-dihydro-1H-indene-2-carboxamide;
(S)-N-(8,9-difluoro-6-oxo-1,4,5,6-tetrahydro-2H-pyrano[3,4-c]isoquinolin-1-yl)-N,2-dimethylindolizine-6-carboxamide;
(R)-N-(8,9-difluoro-6-oxo-1,4,5,6-tetrahydro-2H-pyrano[3,4-c]isoquinolin-1-yl)-N,2-dimethylindolizine-6-carboxamide;
(S)-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
N-((S)-8,9-difluoro-6-oxo-1,4,5,6-tetrahydro-2H-pyrano[3,4-c]isoquinolin-1-yl)-4-fluoro-N-methylbicyclo[4.2.0]octa-1(6),2,4-triene-7-carboxamide;
N-((R)-8,9-difluoro-6-oxo-1,4,5,6-tetrahydro-2H-pyrano[3,4-c]isoquinolin-1-yl)-4-fluoro-N-methylbicyclo[4.2.0]octa-1(6),2,4-triene-7-carboxamide;
(S)-N-(6-(2-aminoethoxy)-8,9-difluoro-1,4-dihydro-2H-pyrano[3,4-c]isoquinolin-1-yl)-5,6-difluoro-N-methyl-1H-indole-2-carboxamide;
(R)-N-(6-(2-aminoethoxy)-8,9-difluoro-1,4-dihydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-1-(thiophen-3-yl)azetidine-3-carboxamide;
(R)-N-(8,9-difluoro-6-oxo-1,4,5,6-tetrahydro-2H-pyrano[3,4-c]isoquinolin-1-yl)-N-methyl-1-(thiophen-3-yl)azetidine-3-carboxamide;
(S)-N-(8,9-difluoro-6-oxo-1,4,5,6-tetrahydro-2H-pyrano[3,4-c]isoquinolin-1-yl)-3′-fluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-3′-fluoro-N-methyl-[1,1′-biphenyl]-4-carboxamide;
(S)-1-(4-bromothiophen-3-yl)-N-(8,9-difluoro-6-oxo-1,4,5,6-tetrahydro-2H-pyrano[3,4-c]isoquinolin-1-yl)-N-methylazetidine-3-carboxamide;
(R)-1-(4-bromothiophen-3-yl)-N-(8,9-difluoro-6-oxo-1,4,5,6-tetrahydro-2H-pyrano[3,4-c]isoquinolin-1-yl)-N-methylazetidine-3-carboxamide;
(S)-N-(8,9-difluoro-6-oxo-1,4,5,6-tetrahydro-2H-pyrano[3,4-c]isoquinolin-1-yl)-2′-fluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-2′-fluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-3′,5′-difluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-3′,5′-difluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-2,3′,5′-trifluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-2,3′,5′-trifluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-N-methyl-[1,1′-biphenyl]-3-carboxamide;
(R)-N-(8,9-difluoro-6-oxo-1,4,5,6-tetrahydro-2H-pyrano[3,4-c]isoquinolin-1-yl)-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-4′-fluoro-N-methyl-[1,1′-biphenyl]-3-carboxamide;
(R)-N-(8,9-difluoro-6-oxo-1,4,5,6-tetrahydro-2H-pyrano[3,4-c]isoquinolin-1-yl)-4′-fluoro-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-3′-fluoro-N-methyl-[1,1′-biphenyl]-3-carboxamide;
(R)-N-(8,9-difluoro-6-oxo-1,4,5,6-tetrahydro-2H-pyrano[3,4-c]isoquinolin-1-yl)-3′-fluoro-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-3′,5-difluoro-N-methyl-[1,1′-biphenyl]-3-carboxamide;
(R)-N-(8,9-difluoro-6-oxo-1,4,5,6-tetrahydro-2H-pyrano[3,4-c]isoquinolin-1-yl)-3′,5-difluoro-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-N-methyl-3-phenoxybenzamide;
(R)-N-(8,9-difluoro-6-oxo-1,4,5,6-tetrahydro-2H-pyrano[3,4-c]isoquinolin-1-yl)-N-methyl-3-phenoxybenzamide;
(S)-N-(8,9-difluoro-6-oxo-1,4,5,6-tetrahydro-2H-pyrano[3,4-c]isoquinolin-1-yl)-3-(4-fluorophenoxy)-N-methylbenzamide;
(R)-N-(8,9-difluoro-6-oxo-1,4,5,6-tetrahydro-2H-pyrano[3,4-c]isoquinolin-1-yl)-3-(4-fluorophenoxy)-N-methylbenzamide;
(S)-N-(8,9-difluoro-6-oxo-1,4,5,6-tetrahydro-2H-pyrano[3,4-c]isoquinolin-1-yl)-N-methyl-6H-thieno[2,3-b]pyrrole-5-carboxamide;
(R)-N-(8,9-difluoro-6-oxo-1,4,5,6-tetrahydro-2H-pyrano[3,4-c]isoquinolin-1-yl)-N-methyl-6H-thieno[2,3-b]pyrrole-5-carboxamide;
(S)-N-(8,9-difluoro-6-oxo-1,4,5,6-tetrahydro-2H-pyrano[3,4-c]isoquinolin-1-yl)-N-methyl-4-(methylsulfonyl)benzamide;
(R)-N-(8,9-difluoro-6-oxo-1,4,5,6-tetrahydro-2H-pyrano[3,4-c]isoquinolin-1-yl)-N-methyl-4-(methylsulfonyl)benzamide;
(S)-N-(8,9-difluoro-6-oxo-1,4,5,6-tetrahydro-2H-pyrano[3,4-c]isoquinolin-1-yl)-N-methyl-3-(N-methylsulfamoyl)benzamide;
(R)-N-(8,9-difluoro-6-oxo-1,4,5,6-tetrahydro-2H-pyrano[3,4-c]isoquinolin-1-yl)-N-methyl-3-(N-methylsulfamoyl)benzamide;
(S)-N-(8,9-difluoro-6-oxo-1,4,5,6-tetrahydro-2H-pyrano[3,4-c]isoquinolin-1-yl)-N-methyl-3-(methylsulfonamido)benzamide;
(R)-N-(8,9-difluoro-6-oxo-1,4,5,6-tetrahydro-2H-pyrano[3,4-c]isoquinolin-1-yl)-N-methyl-3-(methylsulfonamido)benzamide;
(S)-N-(8,9-difluoro-6-oxo-1,4,5,6-tetrahydro-2H-pyrano[3,4-c]isoquinolin-1-yl)-N-methyl-3-(methylsulfonyl)benzamide;
(R)-N-(8,9-difluoro-6-oxo-1,4,5,6-tetrahydro-2H-pyrano[3,4-c]isoquinolin-1-yl)-N-methyl-3-(methylsulfonyl)benzamide;
(S)-N-(8,9-difluoro-6-oxo-1,4,5,6-tetrahydro-2H-pyrano[3,4-c]isoquinolin-1-yl)-N-methylimidazo[1,5-a]pyridine-6-carboxamide;
(R)-N-(8,9-difluoro-6-oxo-1,4,5,6-tetrahydro-2H-pyrano[3,4-c]isoquinolin-1-yl)-N-methylimidazo[1,5-a]pyridine-6-carboxamide;
(1S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1S,4S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1S,4R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1R,4R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1R,4S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(S)-N-(8,9-difluoro-6-oxo-1,2,3,4,5,6-hexahydrobenzo[c][1,7]naphthyridin-1-yl)-3′-fluoro-N-methyl-[1,1′-biphenyl]-4-carboxamide;
(R)-N-(8,9-difluoro-6-oxo-1,2,3,4,5,6-hexahydrobenzo[c][1,7]naphthyridin-1-yl)-3′-fluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-3-fluoro-N-methyl-5-phenoxybenzamide;
(R)-N-(8,9-difluoro-6-oxo-1,4,5,6-tetrahydro-2H-pyrano[3,4-c]isoquinolin-1-yl)-3-fluoro-N-methyl-5-phenoxybenzamide;
(S)-N-(8,9-difluoro-6-oxo-1,2,3,4,5,6-hexahydrobenzo[c][1,7]naphthyridin-1-yl)-3′-fluoro-N-methyl-[1,1′-biphenyl]-3-carboxamide;
(R)-N-(8,9-difluoro-6-oxo-1,2,3,4,5,6-hexahydrobenzo[c][1,7]naphthyridin-1-yl)-3′-fluoro-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-N-methylimidazo[1,2-a]pyridine-6-carboxamide;
(R)-N-(8,9-difluoro-6-oxo-1,4,5,6-tetrahydro-2H-pyrano[3,4-c]isoquinolin-1-yl)-N-methylimidazo[1,2-a]pyridine-6-carboxamide;
(S)-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(R)-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(S)-6-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(R)-6-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(S)-N-(8,9-difluoro-6-oxo-1,4,5,6-tetrahydro-2H-pyrano[3,4-c]isoquinolin-1-yl)-5-fluoro-N-methyl-1H-pyrrolo[2,3-c]pyridine-2-carboxamide;
(R)-N-(8,9-difluoro-6-oxo-1,4,5,6-tetrahydro-2H-pyrano[3,4-c]isoquinolin-1-yl)-5-fluoro-N-methyl-1H-pyrrolo[2,3-c]pyridine-2-carboxamide;
(S)-N-(8,9-difluoro-6-oxo-1,4,5,6-tetrahydro-2H-pyrano[3,4-c]isoquinolin-1-yl)-N-methyl-2-(trifluoromethyl)indolizine-6-carboxamide;
(R)-N-(8,9-difluoro-6-oxo-1,4,5,6-tetrahydro-2H-pyrano[3,4-c]isoquinolin-1-yl)-N-methyl-2-(trifluoromethyl)indolizine-6-carboxamide;
(S)-4-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-4-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-N-(8,9-difluoro-6-oxo-1,4,5,6-tetrahydro-2H-pyrano[3,4-c]isoquinolin-1-yl)-2-(difluoromethyl)-N-methylisonicotinamide;
(R)-N-(8,9-difluoro-6-oxo-1,4,5,6-tetrahydro-2H-pyrano[3,4-c]isoquinolin-1-yl)-2-(difluoromethyl)-N-methylisonicotinamide;
(S)-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
(R)-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
(S)-N-(8,9-difluoro-5-(2-hydroxyethyl)-6-oxo-1,4,5,6-tetrahydro-2H-pyrano[3,4-c]isoquinolin-1-yl)-5,6-difluoro-N-methyl-1H-indole-2-carboxamide;
(R)-N-(8,9-difluoro-5-(2-hydroxyethyl)-6-oxo-1,4,5,6-tetrahydro-2H-pyrano[3,4-c]isoquinolin-1-yl)-5,6-difluoro-N-methyl-1H-indole-2-carboxamide;
(S)-N-(5-(2-aminoethyl)-8,9-difluoro-6-oxo-1,4,5,6-tetrahydro-2H-pyrano[3,4-c]isoquinolin-1-yl)-5,6-difluoro-N-methyl-1H-indole-2-carboxamide;
(R)-N-(5-(2-aminoethyl)-8,9-difluoro-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-(2-hydroxyethoxy)-1,4-dihydro-2H-pyrano[3,4-c]isoquinolin-1-yl)-5,6-difluoro-N-methyl-1H-indole-2-carboxamide;
(R)-N-(8,9-difluoro-6-(2-hydroxyethoxy)-1,4-dihydro-2H-pyrano[3,4-c]isoquinolin-1-yl)-5,6-difluoro-N-methyl-1H-indole-2-carboxamide;
(S)-N-(8,9-difluoro-6-oxo-1,2,3,4,5,6-hexahydrobenzo[c][1,7]naphthyridin-1-yl)-N-methyl-3-(N-methylsulfamoyl)benzamide;
(R)-N-(8,9-difluoro-6-oxo-1,2,3,4,5,6-hexahydrobenzo[c][1,7]naphthyridin-1-yl)-N-methyl-3-(N-methylsulfamoyl)benzamide;
N-((1S)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1S,4R)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1S,4S)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1R)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1R,4R)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1R,4S)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-4,6-dioxo-1,4,5,6-tetrahydro-2H-pyrano[3,4-c]isoquinolin-1-yl)-5,6-difluoro-N-methyl-1H-indole-2-carboxamide;
(R)-N-(8,9-difluoro-4,6-dioxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-3-(3-fluorophenoxy)-N-methylbenzamide;
(R)-N-(8,9-difluoro-6-oxo-1,4,5,6-tetrahydro-2H-pyrano[3,4-c]isoquinolin-1-yl)-3-(3-fluorophenoxy)-N-methylbenzamide;
(S)-N-(8,9-difluoro-6-oxo-1,4,5,6-tetrahydro-2H-pyrano[3,4-c]isoquinolin-1-yl)-5-methoxy-N-methyl-1H-indole-2-carboxamide;
(R)-N-(8,9-difluoro-6-oxo-1,4,5,6-tetrahydro-2H-pyrano[3,4-c]isoquinolin-1-yl)-5-methoxy-N-methyl-1H-indole-2-carboxamide;
(S)-5-chloro-N-(8,9-difluoro-6-oxo-1,4,5,6-tetrahydro-2H-pyrano[3,4-c]isoquinolin-1-yl)-N-methyl-1H-indole-2-carboxamide;
(R)-5-chloro-N-(8,9-difluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N,5-dimethyl-1H-indole-2-carboxamide;
(R)-N-(8,9-difluoro-6-oxo-1,4,5,6-tetrahydro-2H-pyrano[3,4-c]isoquinolin-1-yl)-N,5-dimethyl-1H-indole-2-carboxamide;
(S)-N-(8,9-difluoro-6-oxo-1,4,5,6-tetrahydro-2H-pyrano[3,4-c]isoquinolin-1-yl)-N-methyl-5-(trifluoromethoxy)-1H-indole-2-carboxamide;
(R)-N-(8,9-difluoro-6-oxo-1,4,5,6-tetrahydro-2H-pyrano[3,4-c]isoquinolin-1-yl)-N-methyl-5-(trifluoromethoxy)-1H-indole-2-carboxamide;
(S)-N-(8,9-difluoro-6-oxo-1,4,5,6-tetrahydro-2H-pyrano[3,4-c]isoquinolin-1-yl)-5-ethyl-N-methyl-1H-indole-2-carboxamide;
(R)-N-(8,9-difluoro-6-oxo-1,4,5,6-tetrahydro-2H-pyrano[3,4-c]isoquinolin-1-yl)-5-ethyl-N-methyl-1H-indole-2-carboxamide;
(S)-N-(8,9-difluoro-6-oxo-1,4,5,6-tetrahydro-2H-pyrano[3,4-c]isoquinolin-1-yl)-4-methoxy-N-methyl-1H-indole-2-carboxamide;
(R)-N-(8,9-difluoro-6-oxo-1,4,5,6-tetrahydro-2H-pyrano[3,4-c]isoquinolin-1-yl)-4-methoxy-N-methyl-1H-indole-2-carboxamide;
(S)-N-(8,9-difluoro-6-oxo-1,4,5,6-tetrahydro-2H-pyrano[3,4-c]isoquinolin-1-yl)-N,4-dimethyl-1H-indole-2-carboxamide;
(R)-N-(8,9-difluoro-6-oxo-1,4,5,6-tetrahydro-2H-pyrano[3,4-c]isoquinolin-1-yl)-N,4-dimethyl-1H-indole-2-carboxamide;
(1S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1S,4S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1S,4R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1R,4S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1R,4R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(S)-2-chloro-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
(R)-2-chloro-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
(S)-N-(8,9-difluoro-6-(methylamino)-1,4-dihydro-2H-pyrano[3,4-c]isoquinolin-1-yl)-5,6-difluoro-N-methyl-1H-indole-2-carboxamide;
(R)-N-(8,9-difluoro-6-(methylamino)-1,4-dihydro-2H-pyrano[3,4-c]isoquinolin-1-yl)-5,6-difluoro-N-methyl-1H-indole-2-carboxamide;
(S)-N-(8,9-difluoro-6-((2-hydroxyethyl)amino)-1,4-dihydro-2H-pyrano[3,4-c]isoquinolin-1-yl)-5,6-difluoro-N-methyl-1H-indole-2-carboxamide;
(R)-N-(8,9-difluoro-6-((2-hydroxyethyl)amino)-1,4-dihydro-2H-pyrano[3,4-c]isoquinolin-1-yl)-5,6-difluoro-N-methyl-1H-indole-2-carboxamide;
(S)-N-(8,9-difluoro-6-oxo-1,2,3,4,5,6-hexahydrobenzo[c][1,7]naphthyridin-1-yl)-3-(3-fluorophenoxy)-N-methylbenzamide;
(R)-N-(8,9-difluoro-6-oxo-1,2,3,4,5,6-hexahydrobenzo[c][1,7]naphthyridin-1-yl)-3-(3-fluorophenoxy)-N-methylbenzamide;
(S)-4-cyano-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(R)-4-cyano-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-4-ethyl-6-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4-ethyl-6-fluoro-N-methyl-1H-indole-2-carboxamide;
(S)-4-chloro-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(R)-4-chloro-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-6-fluoro-N,4-dimethyl-1H-indole-2-carboxamide;
(R)-N-(8,9-difluoro-6-oxo-1,4,5,6-tetrahydro-2H-pyrano[3,4-c]isoquinolin-1-yl)-6-fluoro-N,4-dimethyl-1H-indole-2-carboxamide;
(S)-5-cyano-N-(8,9-difluoro-6-oxo-1,4,5,6-tetrahydro-2H-pyrano[3,4-c]isoquinolin-1-yl)-N-methyl-1H-indole-2-carboxamide;
(R)-5-cyano-N-(8,9-difluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-5-(methylsulfonyl)-1H-indole-2-carboxamide;
(R)-N-(8,9-difluoro-6-oxo-1,4,5,6-tetrahydro-2H-pyrano[3,4-c]isoquinolin-1-yl)-N-methyl-5-(methylsulfonyl)-1H-indole-2-carboxamide;
(S)-4-chloro-N-(8,9-difluoro-6-oxo-1,4,5,6-tetrahydro-2H-pyrano[3,4-c]isoquinolin-1-yl)-N-methyl-1H-indole-2-carboxamide;
(R)-4-chloro-N-(8,9-difluoro-6-oxo-1,4,5,6-tetrahydro-2H-pyrano[3,4-c]isoquinolin-1-yl)-N-methyl-1H-indole-2-carboxamide;
(S)-6-bromo-N-(8,9-difluoro-6-oxo-1,4,5,6-tetrahydro-2H-pyrano[3,4-c]isoquinolin-1-yl)-N-methylindolizine-2-carboxamide;
(R)-6-bromo-N-(8,9-difluoro-6-oxo-1,4,5,6-tetrahydro-2H-pyrano[3,4-c]isoquinolin-1-yl)-N-methylindolizine-2-carboxamide;
(S)-2-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-2-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-3-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-3-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-N-(6-((2-aminoethyl)amino)-8,9-difluoro-1,4-dihydro-2H-pyrano[3,4-c]isoquinolin-1-yl)-5,6-difluoro-N-methyl-1H-indole-2-carboxamide;
(R)-N-(6-((2-aminoethyl)amino)-8,9-difluoro-1,4-dihydro-2H-pyrano[3,4-c]isoquinolin-1-yl)-5,6-difluoro-N-methyl-1H-indole-2-carboxamide;
N-((S)-8,9-difluoro-6-oxo-1,4,5,6-tetrahydro-2H-pyrano[3,4-c]isoquinolin-1-yl)-6-fluoro-4-(1-hydroxyethyl)-N-methyl-1H-indole-2-carboxamide;
N-((R)-8,9-difluoro-6-oxo-1,4,5,6-tetrahydro-2H-pyrano[3,4-c]isoquinolin-1-yl)-6-fluoro-4-(1-hydroxyethyl)-N-methyl-1H-indole-2-carboxamide;
N-((S)-8,9-difluoro-6-oxo-1,4,5,6-tetrahydro-2H-pyrano[3,4-c]isoquinolin-1-yl)-6-fluoro-4-((S)-1-hydroxyethyl)-N-methyl-1H-indole-2-carboxamide;
N-((R)-8,9-difluoro-6-oxo-1,4,5,6-tetrahydro-2H-pyrano[3,4-c]isoquinolin-1-yl)-6-fluoro-4-((S)-1-hydroxyethyl)-N-methyl-1H-indole-2-carboxamide;
N-((S)-8,9-difluoro-6-oxo-1,4,5,6-tetrahydro-2H-pyrano[3,4-c]isoquinolin-1-yl)-6-fluoro-4-((R)-1-hydroxyethyl)-N-methyl-1H-indole-2-carboxamide;
N-((S)-8,9-difluoro-6-oxo-1,4,5,6-tetrahydro-2H-pyrano[3,4-c]isoquinolin-1-yl)-6-fluoro-4-((S)-1-hydroxyethyl)-N-methyl-1H-indole-2-carboxamide;
(S)-N-(8,9-difluoro-6-oxo-1,4,5,6-tetrahydro-2H-pyrano[3,4-c]isoquinolin-1-yl)-6-fluoro-4-(hydroxymethyl)-N-methyl-1H-indole-2-carboxamide;
(R)-N-(8,9-difluoro-6-oxo-1,4,5,6-tetrahydro-2H-pyrano[3,4-c]isoquinolin-1-yl)-6-fluoro-4-(hydroxymethyl)-N-methyl-1H-indole-2-carboxamide;
(S)-7-bromo-N-(8,9-difluoro-6-oxo-1,4,5,6-tetrahydro-2H-pyrano[3,4-c]isoquinolin-1-yl)-N-methylfuro[3,2-c]pyridine-2-carboxamide;
(R)-7-bromo-N-(8,9-difluoro-6-oxo-1,4,5,6-tetrahydro-2H-pyrano[3,4-c]isoquinolin-1-yl)-N-methylfuro[3,2-c]pyridine-2-carboxamide;
(S)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-6-carboxamide;
(R)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-6-carboxamide;
(S)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-6-carboxamide;
(R)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-6-carboxamide;
(S)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
(R)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
(S)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-5-carboxamide;
(R)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-5-carboxamide;
(S)-3,4-dichloro-N-(8,9-difluoro-6-oxo-1,4,5,6-tetrahydro-2H-pyrano[3,4-c]isoquinolin-1-yl)-N-methylbenzamide;
(R)-3,4-dichloro-N-(8,9-difluoro-6-oxo-1,4,5,6-tetrahydro-2H-pyrano[3,4-c]isoquinolin-1-yl)-N-methylbenzamide;
(S)-N-(8,9-difluoro-6-oxo-1,4,5,6-tetrahydro-2H-pyrano[3,4-c]isoquinolin-1-yl)-6-fluoro-N-methyl-4-(methylsulfonamido)-1H-indole-2-carboxamide;
(R)-N-(8,9-difluoro-6-oxo-1,4,5,6-tetrahydro-2H-pyrano[3,4-c]isoquinolin-1-yl)-6-fluoro-N-methyl-4-(methylsulfonamido)-1H-indole-2-carboxamide;
(S)-N-(8,9-difluoro-6-oxo-1,4,5,6-tetrahydro-2H-pyrano[3,4-c]isoquinolin-1-yl)-N-methyl-4-(trifluoromethoxy)-1H-indole-2-carboxamide;
(R)-N-(8,9-difluoro-6-oxo-1,4,5,6-tetrahydro-2H-pyrano[3,4-c]isoquinolin-1-yl)-N-methyl-4-(trifluoromethoxy)-1H-indole-2-carboxamide;
(S)-N-(8,9-difluoro-6-oxo-1,4,5,6-tetrahydro-2H-pyrano[3,4-c]isoquinolin-1-yl)-6-methoxy-N-methyl-1H-indole-2-carboxamide;
(R)-N-(8,9-difluoro-6-oxo-1,4,5,6-tetrahydro-2H-pyrano[3,4-c]isoquinolin-1-yl)-6-methoxy-N-methyl-1H-indole-2-carboxamide;
(S)-5-bromo-N-(8,9-difluoro-6-oxo-1,4,5,6-tetrahydro-2H-pyrano[3,4-c]isoquinolin-1-yl)-N-methyl-6-(trifluoromethyl)nicotinamide;
(R)-5-bromo-N-(8,9-difluoro-6-oxo-1,4,5,6-tetrahydro-2H-pyrano[3,4-c]isoquinolin-1-yl)-N-methyl-6-(trifluoromethyl)nicotinamide;
(S)-3-chloro-N-(8,9-difluoro-6-oxo-1,4,5,6-tetrahydro-2H-pyrano[3,4-c]isoquinolin-1-yl)-N-methylindolizine-6-carboxamide;
(R)-3-chloro-N-(8,9-difluoro-6-oxo-1,4,5,6-tetrahydro-2H-pyrano[3,4-c]isoquinolin-1-yl)-N-methylindolizine-6-carboxamide;
(S)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-6-carboxamide;
(R)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-6-carboxamide;
(S)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-5-carboxamide;
(R)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-5-carboxamide;
(S)-N-(8,9-difluoro-6-oxo-1,4,5,6-tetrahydro-2H-pyrano[3,4-c]isoquinolin-1-yl)-3-methoxy-N-methyl-1H-indazole-5-carboxamide;
(R)-N-(8,9-difluoro-6-oxo-1,4,5,6-tetrahydro-2H-pyrano[3,4-c]isoquinolin-1-yl)-3-methoxy-N-methyl-1H-indazole-5-carboxamide;
(S)-1-(5-chlorothiophen-3-yl)-N-(8,9-difluoro-6-oxo-1,4,5,6-tetrahydro-2H-pyrano[3,4-c]isoquinolin-1-yl)-N-methylazetidine-3-carboxamide;
(R)-1-(5-chlorothiophen-3-yl)-N-(8,9-difluoro-6-oxo-1,4,5,6-tetrahydro-2H-pyrano[3,4-c]isoquinolin-1-yl)-N-methylazetidine-3-carboxamide;
(S)-7-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-7-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-4-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(R)-4-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(S)-3-chloro-N-(8,9-difluoro-6-oxo-1,4,5,6-tetrahydro-2H-pyrano[3,4-c]isoquinolin-1-yl)-N-methylindolizine-7-carboxamide;
(R)-3-chloro-N-(8,9-difluoro-6-oxo-1,4,5,6-tetrahydro-2H-pyrano[3,4-c]isoquinolin-1-yl)-N-methylindolizine-7-carboxamide;
(S)-N-(8,9-difluoro-6-oxo-1,4,5,6-tetrahydro-2H-pyrano[3,4-c]isoquinolin-1-yl)-7-(difluoromethyl)-N-methylindolizine-2-carboxamide;
(R)-N-(8,9-difluoro-6-oxo-1,4,5,6-tetrahydro-2H-pyrano[3,4-c]isoquinolin-1-yl)-7-(difluoromethyl)-N-methylindolizine-2-carboxamide;
(S)-N-(8,9-difluoro-6-oxo-1,4,5,6-tetrahydro-2H-pyrano[3,4-c]isoquinolin-1-yl)-6-(difluoromethyl)-N-methylindolizine-2-carboxamide;
(R)-N-(8,9-difluoro-6-oxo-1,4,5,6-tetrahydro-2H-pyrano[3,4-c]isoquinolin-1-yl)-6-(difluoromethyl)-N-methylindolizine-2-carboxamide;
(S)-di-tert-butyl ((2-((8,9-difluoro-6-oxo-1,4,5,6-tetrahydro-2H-pyrano[3,4-c]isoquinolin-1-yl)(methyl)carbamoyl)-5,6-difluoro-1H-indol-1-yl)methyl)phosphate;
(R)-di-tert-butyl ((2-((8,9-difluoro-6-oxo-1,4,5,6-tetrahydro-2H-pyrano[3,4-c]isoquinolin-1-yl)(methyl)carbamoyl)-5,6-difluoro-1H-indol-1-yl)methyl)phosphate;
(S)-5-chloro-N-(8,9-difluoro-6-oxo-1,4,5,6-tetrahydro-2H-pyrano[3,4-c]isoquinolin-1-yl)-N-methyl-6-(trifluoromethyl)nicotinamide;
(R)-5-chloro-N-(8,9-difluoro-6-oxo-1,4,5,6-tetrahydro-2H-pyrano[3,4-c]isoquinolin-1-yl)-N-methyl-6-(trifluoromethyl)nicotinamide;
(S)-5-chloro-N-(8-fluoro-6-oxo-1,4,5,6-tetrahydro-2H-pyrano[3,4-c]isoquinolin-1-yl)-N-methyl-6-(trifluoromethyl)nicotinamide;
(R)-5-chloro-N-(8-fluoro-6-oxo-1,4,5,6-tetrahydro-2H-pyrano[3,4-c]isoquinolin-1-yl)-N-methyl-6-(trifluoromethyl)nicotinamide;
(S)-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-4-cyano-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-cyano-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-4-bromo-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-4-bromo-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-N-(8,9-difluoro-6-oxo-1,2,3,4,5,6-hexahydrobenzo[c][1,7]naphthyridin-1-yl)-7-(difluoromethyl)-N-methylindolizine-2-carboxamide;
(R)-N-(8,9-difluoro-6-oxo-1,2,3,4,5,6-hexahydrobenzo[c][1,7]naphthyridin-1-yl)-7-(difluoromethyl)-N-methylindolizine-2-carboxamide;
(S)-N-(8,9-difluoro-6-oxo-1,2,3,4,5,6-hexahydrobenzo[c][1,7]naphthyridin-1-yl)-6-(difluoromethyl)-N-methylindolizine-2-carboxamide;
(R)-N-(8,9-difluoro-6-oxo-1,2,3,4,5,6-hexahydrobenzo[c][1,7]naphthyridin-1-yl)-6-(difluoromethyl)-N-methylindolizine-2-carboxamide;
(S)-N-(8-fluoro-6-oxo-1,4,5,6-tetrahydro-2H-pyrano[3,4-c]isoquinolin-1-yl)-N-methylindolizine-6-carboxamide;
(R)-N-(8-fluoro-6-oxo-1,4,5,6-tetrahydro-2H-pyrano[3,4-c]isoquinolin-1-yl)-N-methylindolizine-6-carboxamide;
(S)-N-(8,9-difluoro-6-oxo-1,2,3,4,5,6-hexahydrobenzo[c][1,7]naphthyridin-1-yl)-N-methylindolizine-6-carboxamide;
(R)-N-(8,9-difluoro-6-oxo-1,2,3,4,5,6-hexahydrobenzo[c][1,7]naphthyridin-1-yl)-N-methylindolizine-6-carboxamide;
(S)-4-ethyl-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-ethyl-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-5-cyano-N-(8-fluoro-6-oxo-1,4,5,6-tetrahydro-2H-pyrano[3,4-c]isoquinolin-1-yl)-N-methyl-1H-indole-2-carboxamide;
(R)-5-cyano-N-(8-fluoro-6-oxo-1,4,5,6-tetrahydro-2H-pyrano[3,4-c]isoquinolin-1-yl)-N-methyl-1H-indole-2-carboxamide;
(S)-4-cyano-N-(8,9-difluoro-6-oxo-1,2,3,4,5,6-hexahydrobenzo[c][1,7]naphthyridin-1-yl)-6-fluoro-N-methyl-1H-indole-2-carboxamide;
(R)-4-cyano-N-(8,9-difluoro-6-oxo-1,2,3,4,5,6-hexahydrobenzo[c][1,7]naphthyridin-1-yl)-6-fluoro-N-methyl-1H-indole-2-carboxamide;
(S)-N-(8,9-difluoro-6-oxo-1,2,3,4,5,6-hexahydrobenzo[c][1,7]naphthyridin-1-yl)-4-ethyl-6-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-4-ethyl-6-fluoro-N-methyl-1H-indole-2-carboxamide;
(S)-N-(8,9-difluoro-6-oxo-1,2,3,4,5,6-hexahydrobenzo[c][1,7]naphthyridin-1-yl)-N-methyl-5-(methylsulfonyl)-1H-indole-2-carboxamide;
(R)-N-(8,9-difluoro-6-oxo-1,2,3,4,5,6-hexahydrobenzo[c][1,7]naphthyridin-1-yl)-N-methyl-5-(methylsulfonyl)-1H-indole-2-carboxamide;
(S)-5-cyano-N-(8,9-difluoro-6-oxo-1,2,3,4,5,6-hexahydrobenzo[c][1,7]naphthyridin-1-yl)-N-methyl-1H-indole-2-carboxamide;
(R)-5-cyano-N-(8,9-difluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-8-(difluoromethyl)-N-methylindolizine-2-carboxamide;
(R)-N-(8,9-difluoro-6-oxo-1,4,5,6-tetrahydro-2H-pyrano[3,4-c]isoquinolin-1-yl)-8-(difluoromethyl)-N-methylindolizine-2-carboxamide;
(S)-N-(8,9-difluoro-6-oxo-1,4,5,6-tetrahydro-2H-pyrano[3,4-c]isoquinolin-1-yl)-N-methyl-2,3-dihydro-1H-indene-5-carboxamide;
(R)-N-(8,9-difluoro-6-oxo-1,4,5,6-tetrahydro-2H-pyrano[3,4-c]isoquinolin-1-yl)-N-methyl-2,3-dihydro-1H-indene-5-carboxamide;
(S)-N-(8,9-difluoro-6-oxo-1,4,5,6-tetrahydro-2H-pyrano[3,4-c]isoquinolin-1-yl)-N-methylbenzo[d]oxazole-6-carboxamide;
(R)-N-(8,9-difluoro-6-oxo-1,4,5,6-tetrahydro-2H-pyrano[3,4-c]isoquinolin-1-yl)-N-methylbenzo[d]oxazole-6-carboxamide;
(S)-4,6-dichloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(R)-4,6-dichloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
5,6-difluoro-N-methyl-N-((1S)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1S,4S)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1S,4R)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1R)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1R,4S)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1R,4R)-4,8,9-trifluoro-6-oxo-1,4,5,6-tetrahydro-2H-pyrano[3,4-c]isoquinolin-1-yl)-1H-indole-2-carboxamide;
(S)-N-(8,9-difluoro-6-oxo-1,4,5,6-tetrahydro-2H-pyrano[3,4-c]isoquinolin-1-yl)-5-fluoro-N-methyl-6-(trifluoromethyl)nicotinamide;
(R)-N-(8,9-difluoro-6-oxo-1,4,5,6-tetrahydro-2H-pyrano[3,4-c]isoquinolin-1-yl)-5-fluoro-N-methyl-6-(trifluoromethyl)nicotinamide;
(S)-5-fluoro-N-(8-fluoro-6-oxo-1,4,5,6-tetrahydro-2H-pyrano[3,4-c]isoquinolin-1-yl)-N-methyl-6-(trifluoromethyl)nicotinamide;
(R)-5-fluoro-N-(8-fluoro-6-oxo-1,4,5,6-tetrahydro-2H-pyrano[3,4-c]isoquinolin-1-yl)-N-methyl-6-(trifluoromethyl)nicotinamide;
(S)-3-chloro-N-(8,9-difluoro-6-oxo-1,4,5,6-tetrahydro-2H-pyrano[3,4-c]isoquinolin-1-yl)-N,1-dimethyl-1H-indazole-5-carboxamide;
(R)-3-chloro-N-(8,9-difluoro-6-oxo-1,4,5,6-tetrahydro-2H-pyrano[3,4-c]isoquinolin-1-yl)-N,1-dimethyl-1H-indazole-5-carboxamide;
(S)-3-chloro-N-(8,9-difluoro-6-oxo-1,4,5,6-tetrahydro-2H-pyrano[3,4-c]isoquinolin-1-yl)-1-ethyl-N-methyl-1H-indazole-5-carboxamide;
(R)-3-chloro-N-(8,9-difluoro-6-oxo-1,4,5,6-tetrahydro-2H-pyrano[3,4-c]isoquinolin-1-yl)-1-ethyl-N-methyl-1H-indazole-5-carboxamide;
(S)-N-(8,9-difluoro-6-oxo-1,4,5,6-tetrahydro-2H-pyrano[3,4-c]isoquinolin-1-yl)-1-(difluoromethyl)-N,3-dimethyl-1H-indazole-5-carboxamide;
(R)-N-(8,9-difluoro-6-oxo-1,4,5,6-tetrahydro-2H-pyrano[3,4-c]isoquinolin-1-yl)-1-(difluoromethyl)-N,3-dimethyl-1H-indazole-5-carboxamide;
(S)-5-chloro-N-(8,9-difluoro-6-oxo-1,2,3,4,5,6-hexahydrobenzo[c][1,7]naphthyridin-1-yl)-N-methyl-6-(trifluoromethyl)nicotinamide;
(R)-5-chloro-N-(8,9-difluoro-6-oxo-1,2,3,4,5,6-hexahydrobenzo[c][1,7]naphthyridin-1-yl)-N-methyl-6-(trifluoromethyl)nicotinamide;
(S)-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
(R)-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
(S)-N-(8-fluoro-6-oxo-1,4,5,6-tetrahydro-2H-pyrano[3,4-c]isoquinolin-1-yl)-N,4-dimethyl-1H-indole-2-carboxamide;
(R)-N-(8-fluoro-6-oxo-1,4,5,6-tetrahydro-2H-pyrano[3,4-c]isoquinolin-1-yl)-N,4-dimethyl-1H-indole-2-carboxamide;
(S)-4-chloro-N-(8-fluoro-6-oxo-1,4,5,6-tetrahydro-2H-pyrano[3,4-c]isoquinolin-1-yl)-N-methyl-1H-indole-2-carboxamide;
(R)-4-chloro-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methylbenzo[d]oxazole-5-carboxamide;
(R)-N-(8,9-difluoro-6-oxo-1,4,5,6-tetrahydro-2H-pyrano[3,4-c]isoquinolin-1-yl)-N-methylbenzo[d]oxazole-5-carboxamide;
(S)-N-(8,9-difluoro-6-oxo-1,4,5,6-tetrahydro-2H-pyrano[3,4-c]isoquinolin-1-yl)-N-methylbenzo[d]thiazole-5-carboxamide;
(R)-N-(8,9-difluoro-6-oxo-1,4,5,6-tetrahydro-2H-pyrano[3,4-c]isoquinolin-1-yl)-N-methylbenzo[d]thiazole-5-carboxamide;
(S)-N-(8,9-difluoro-6-oxo-1,4,5,6-tetrahydro-2H-pyrano[3,4-c]isoquinolin-1-yl)-N-methylbenzo[d]thiazole-6-carboxamide;
(R)-N-(8,9-difluoro-6-oxo-1,4,5,6-tetrahydro-2H-pyrano[3,4-c]isoquinolin-1-yl)-N-methylbenzo[d]thiazole-6-carboxamide;
(S)-N-(8,9-difluoro-6-oxo-1,4,5,6-tetrahydro-2H-pyrano[3,4-c]isoquinolin-1-yl)-N-methyl-1H-indazole-5-carboxamide;
(R)-N-(8,9-difluoro-6-oxo-1,4,5,6-tetrahydro-2H-pyrano[3,4-c]isoquinolin-1-yl)-N-methyl-1H-indazole-5-carboxamide;
(S)-N-(8,9-difluoro-6-oxo-1,2,3,4,5,6-hexahydrobenzo[c][1,7]naphthyridin-1-yl)-8-(difluoromethyl)-N-methylindolizine-2-carboxamide;
(R)-N-(8,9-difluoro-6-oxo-1,2,3,4,5,6-hexahydrobenzo[c][1,7]naphthyridin-1-yl)-8-(difluoromethyl)-N-methylindolizine-2-carboxamide;
(S)-N-(8,9-difluoro-6-oxo-1,2,3,4,5,6-hexahydrobenzo[c][1,7]naphthyridin-1-yl)-5-(difluoromethyl)-N-methylindolizine-2-carboxamide;
(R)-N-(8,9-difluoro-6-oxo-1,2,3,4,5,6-hexahydrobenzo[c][1,7]naphthyridin-1-yl)-5-(difluoromethyl)-N-methylindolizine-2-carboxamide;
(S)-N-(8,9-difluoro-6-oxo-1,2,3,4,5,6-hexahydrobenzo[c][1,7]naphthyridin-1-yl)-5-fluoro-N-methyl-6-(trifluoromethyl)nicotinamide;
(R)-N-(8,9-difluoro-6-oxo-1,2,3,4,5,6-hexahydrobenzo[c][1,7]naphthyridin-1-yl)-5-fluoro-N-methyl-6-(trifluoromethyl)nicotinamide;
(S)-N-(8,9-difluoro-6-oxo-1,4,5,6-tetrahydro-2H-pyrano[3,4-c]isoquinolin-1-yl)-3-(difluoromethyl)-N,1-dimethyl-1H-indazole-5-carboxamide;
(R)-N-(8,9-difluoro-6-oxo-1,4,5,6-tetrahydro-2H-pyrano[3,4-c]isoquinolin-1-yl)-3-(difluoromethyl)-N,1-dimethyl-1H-indazole-5-carboxamide;
(S)-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
(R)-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
(S)-4-chloro-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-chloro-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-4-chloro-N-(8,9-difluoro-6-oxo-1,2,3,4,5,6-hexahydrobenzo[c][1,7]naphthyridin-1-yl)-N-methyl-1H-indole-2-carboxamide;
(R)-4-chloro-N-(8,9-difluoro-6-oxo-1,2,3,4,5,6-hexahydrobenzo[c][1,7]naphthyridin-1-yl)-N-methyl-1H-indole-2-carboxamide;
(S)-N-(8-fluoro-6-oxo-1,4,5,6-tetrahydro-2H-pyrano[3,4-c]isoquinolin-1-yl)-N-methyl-2,3-dihydro-1H-indene-5-carboxamide;
(R)-N-(8-fluoro-6-oxo-1,4,5,6-tetrahydro-2H-pyrano[3,4-c]isoquinolin-1-yl)-N-methyl-2,3-dihydro-1H-indene-5-carboxamide;
(S)-N-(8-fluoro-6-oxo-1,4,5,6-tetrahydro-2H-pyrano[3,4-c]isoquinolin-1-yl)-N-methylbenzo[d]thiazole-5-carboxamide;
(R)-N-(8-fluoro-6-oxo-1,4,5,6-tetrahydro-2H-pyrano[3,4-c]isoquinolin-1-yl)-N-methylbenzo[d]thiazole-5-carboxamide;
(S)-N-(8-fluoro-6-oxo-1,4,5,6-tetrahydro-2H-pyrano[3,4-c]isoquinolin-1-yl)-N-methylbenzo[d]thiazole-6-carboxamide;
(R)-N-(8-fluoro-6-oxo-1,4,5,6-tetrahydro-2H-pyrano[3,4-c]isoquinolin-1-yl)-N-methylbenzo[d]thiazole-6-carboxamide;
(S)-N-(8-fluoro-6-oxo-1,4,5,6-tetrahydro-2H-pyrano[3,4-c]isoquinolin-1-yl)-N-methyl-1H-indazole-5-carboxamide;
(R)-N-(8-fluoro-6-oxo-1,4,5,6-tetrahydro-2H-pyrano[3,4-c]isoquinolin-1-yl)-N-methyl-1H-indazole-5-carboxamide;
(S)-N-(8,9-difluoro-6-oxo-1,4,5,6-tetrahydro-2H-pyrano[3,4-c]isoquinolin-1-yl)-N-methyl-1H-indazole-6-carboxamide;
(R)-N-(8,9-difluoro-6-oxo-1,4,5,6-tetrahydro-2H-pyrano[3,4-c]isoquinolin-1-yl)-N-methyl-1H-indazole-6-carboxamide;
(S)-N-(8,9-difluoro-6-oxo-1,4,5,6-tetrahydro-2H-pyrano[3,4-c]isoquinolin-1-yl)-N-methyl-1H-benzo[d]imidazole-6-carboxamide;
(R)-N-(8,9-difluoro-6-oxo-1,4,5,6-tetrahydro-2H-pyrano[3,4-c]isoquinolin-1-yl)-N-methyl-1H-benzo[d]imidazole-6-carboxamide;
(R)-2-chloro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-2-chloro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-2-chloro-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
(S)-2-chloro-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
(R)-6-fluoro-N-(8-fluoro-6-oxo-1,4,5,6-tetrahydro-2H-pyrano[3,4-c]isoquinolin-1-yl)-N,4-dimethyl-1H-indole-2-carboxamide;
(S)-6-fluoro-N-(8-fluoro-6-oxo-1,4,5,6-tetrahydro-2H-pyrano[3,4-c]isoquinolin-1-yl)-N,4-dimethyl-1H-indole-2-carboxamide;
(R)-N-(8,9-difluoro-6-oxo-1,2,3,4,5,6-hexahydrobenzo[c][1,7]naphthyridin-1-yl)-N,4-dimethyl-1H-indole-2-carboxamide;
(S)-N-(8,9-difluoro-6-oxo-1,2,3,4,5,6-hexahydrobenzo[c][1,7]naphthyridin-1-yl)-N,4-dimethyl-1H-indole-2-carboxamide;
(R)-N-(8,9-difluoro-6-oxo-1,2,3,4,5,6-hexahydrobenzo[c][1,7]naphthyridin-1-yl)-6-fluoro-N,4-dimethyl-1H-indole-2-carboxamide;
(S)-N-(8,9-difluoro-6-oxo-1,2,3,4,5,6-hexahydrobenzo[c][1,7]naphthyridin-1-yl)-6-fluoro-N,4-dimethyl-1H-indole-2-carboxamide;
(R)-4-chloro-N-(8,9-difluoro-6-oxo-1,2,3,4,5,6-hexahydrobenzo[c][1,7]naphthyridin-1-yl)-6-fluoro-N-methyl-1H-indole-2-carboxamide;
(S)-4-chloro-N-(8,9-difluoro-6-oxo-1,2,3,4,5,6-hexahydrobenzo[c][1,7]naphthyridin-1-yl)-6-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-N-methylbenzo[d]thiazole-5-carboxamide;
(S)-N-(8,9-difluoro-6-oxo-1,2,3,4,5,6-hexahydrobenzo[c][1,7]naphthyridin-1-yl)-N-methylbenzo[d]thiazole-5-carboxamide;
(R)-N-(8,9-difluoro-6-oxo-1,2,3,4,5,6-hexahydrobenzo[c][1,7]naphthyridin-1-yl)-N-methylbenzo[d]thiazole-6-carboxamide;
(S)-N-(8,9-difluoro-6-oxo-1,2,3,4,5,6-hexahydrobenzo[c][1,7]naphthyridin-1-yl)-N-methylbenzo[d]thiazole-6-carboxamide;
(R)-N-(8,9-difluoro-6-oxo-1,2,3,4,5,6-hexahydrophenanthridin-1-yl)-N-methyl-1H-indole-2-carboxamide;
(S)-N-(8,9-difluoro-6-oxo-1,2,3,4,5,6-hexahydrophenanthridin-1-yl)-N-methyl-1H-indole-2-carboxamide;
(S)-2-cyano-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-2-cyano-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-N-(8-fluoro-6-oxo-1,4,5,6-tetrahydro-2H-pyrano[3,4-c]isoquinolin-1-yl)-N-methyl-1H-indazole-6-carboxamide;
(S)-N-(8-fluoro-6-oxo-1,4,5,6-tetrahydro-2H-pyrano[3,4-c]isoquinolin-1-yl)-N-methyl-1H-indazole-6-carboxamide;
(R)-N-(8,9-difluoro-6-oxo-1,2,3,4,5,6-hexahydrobenzo[c][1,7]naphthyridin-1-yl)-N-methyl-2,3-dihydro-1H-indene-5-carboxamide;
(S)-N-(8,9-difluoro-6-oxo-1,2,3,4,5,6-hexahydrobenzo[c][1,7]naphthyridin-1-yl)-N-methyl-2,3-dihydro-1H-indene-5-carboxamide;
(R)-N-(8,9-difluoro-6-oxo-1,2,3,4,5,6-hexahydrobenzo[c][1,7]naphthyridin-1-yl)-N-methyl-1H-indazole-5-carboxamide;
(S)-N-(8,9-difluoro-6-oxo-1,2,3,4,5,6-hexahydrobenzo[c][1,7]naphthyridin-1-yl)-N-methyl-1H-indazole-5-carboxamide;
(R)-N-(8-fluoro-6-oxo-1,2,3,4,5,6-hexahydrophenanthridin-1-yl)-N-methyl-1H-indole-2-carboxamide;
(S)-N-(8-fluoro-6-oxo-1,2,3,4,5,6-hexahydrophenanthridin-1-yl)-N-methyl-1H-indole-2-carboxamide;
(S)-2-chloro-N-(8,9-difluoro-4,6-dioxo-1,4,5,6-tetrahydro-2H-pyrano[3,4-c]isoquinolin-1-yl)-N-methyl-4H-thieno[3,2-b]pyrrole-5-carboxamide;
(R)-2-chloro-N-(8,9-difluoro-4,6-dioxo-1,4,5,6-tetrahydro-2H-pyrano[3,4-c]isoquinolin-1-yl)-N-methyl-4H-thieno[3,2-b]pyrrole-5-carboxamide;
(S)-N-(8,9-difluoro-4,6-dioxo-1,4,5,6-tetrahydro-2H-pyrano[3,4-c]isoquinolin-1-yl)-6-(difluoromethyl)-5-fluoro-N-methyl-1H-indole-2-carboxamide;
(R)-N-(8,9-difluoro-4,6-dioxo-1,4,5,6-tetrahydro-2H-pyrano[3,4-c]isoquinolin-1-yl)-6-(difluoromethyl)-5-fluoro-N-methyl-1H-indole-2-carboxamide;
(R)-6-(difluoromethyl)-5-fluoro-N-(8-fluoro-6-oxo-1,2,3,4,5,6-hexahydrophenanthridin-1-yl)-N-methyl-1H-indole-2-carboxamide;
(S)-6-(difluoromethyl)-5-fluoro-N-(8-fluoro-6-oxo-1,2,3,4,5,6-hexahydrophenanthridin-1-yl)-N-methyl-1H-indole-2-carboxamide;
(R)-N-(8,9-difluoro-6-oxo-1,2,3,4,5,6-hexahydrophenanthridin-1-yl)-5-fluoro-N-methyl-1H-indole-2-carboxamide;
(S)-N-(8,9-difluoro-6-oxo-1,2,3,4,5,6-hexahydrophenanthridin-1-yl)-5-fluoro-N-methyl-1H-indole-2-carboxamide;
(R)-N-(8,9-difluoro-6-oxo-1,2,3,4,5,6-hexahydrophenanthridin-1-yl)-5,6-difluoro-N-methyl-1H-indole-2-carboxamide;
(S)-N-(8,9-difluoro-6-oxo-1,2,3,4,5,6-hexahydrophenanthridin-1-yl)-5,6-difluoro-N-methyl-1H-indole-2-carboxamide;
(R)-N-(8,9-difluoro-6-oxo-1,2,3,4,5,6-hexahydrophenanthridin-1-yl)-6-(difluoromethyl)-5-fluoro-N-methyl-1H-indole-2-carboxamide;
(S)-N-(8,9-difluoro-6-oxo-1,2,3,4,5,6-hexahydrophenanthridin-1-yl)-6-(difluoromethyl)-5-fluoro-N-methyl-1H-indole-2-carboxamide;
(R)-N-(8,9-difluoro-6-oxo-1,2,3,4,5,6-hexahydrophenanthridin-1-yl)-4-(difluoromethyl)-6-fluoro-N-methyl-1H-indole-2-carboxamide;
(S)-N-(8,9-difluoro-6-oxo-1,2,3,4,5,6-hexahydrophenanthridin-1-yl)-4-(difluoromethyl)-6-fluoro-N-methyl-1H-indole-2-carboxamide;
(R)-N-(8,9-difluoro-6-oxo-1,2,3,4,5,6-hexahydrophenanthridin-1-yl)-N,4-dimethyl-1H-indole-2-carboxamide;
(S)-N-(8,9-difluoro-6-oxo-1,2,3,4,5,6-hexahydrophenanthridin-1-yl)-N,4-dimethyl-1H-indole-2-carboxamide;
(R)-N-(8,9-difluoro-6-oxo-1,2,3,4,5,6-hexahydrophenanthridin-1-yl)-6-fluoro-N,4-dimethyl-1H-indole-2-carboxamide;
(S)-N-(8,9-difluoro-6-oxo-1,2,3,4,5,6-hexahydrophenanthridin-1-yl)-6-fluoro-N,4-dimethyl-1H-indole-2-carboxamide;
(S)-1-((4-bromo-3-fluorobenzyl)(methyl)amino)-8,9-difluoro-1,5-dihydro-2H-pyrano[3,4-c]isoquinolin-6(4H)-one;
(R)-1-((4-bromo-3-fluorobenzyl)(methyl)amino)-8,9-difluoro-1,5-dihydro-2H-pyrano[3,4-c]isoquinolin-6(4H)-one;
2-chloro-N-((1S)-8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
(S,R)-2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
(S,S)-2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
2-chloro-N-((1R)-8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
(R,R)-2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
(R,S)-2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
N-((1R)-8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(R,R)-N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(R,S)-N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
N-((1S)-8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(S,S)-N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(S,R)-N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(R)-5-fluoro-N-(8-fluoro-6-oxo-1,2,3,4,5,6-hexahydrophenanthridin-1-yl)-N-methyl-1H-indole-2-carboxamide;
(S)-5-fluoro-N-(8-fluoro-6-oxo-1,2,3,4,5,6-hexahydrophenanthridin-1-yl)-N-methyl-1H-indole-2-carboxamide;
(R)-5,6-difluoro-N-(8-fluoro-6-oxo-1,2,3,4,5,6-hexahydrophenanthridin-1-yl)-N-methyl-1H-indole-2-carboxamide;
(S)-5,6-difluoro-N-(8-fluoro-6-oxo-1,2,3,4,5,6-hexahydrophenanthridin-1-yl)-N-methyl-1H-indole-2-carboxamide;
(R)-N-(8-fluoro-6-oxo-1,2,3,4,5,6-hexahydrophenanthridin-1-yl)-N,4-dimethyl-1H-indole-2-carboxamide;
(S)-N-(8-fluoro-6-oxo-1,2,3,4,5,6-hexahydrophenanthridin-1-yl)-N,4-dimethyl-1H-indole-2-carboxamide;
(R)-6-fluoro-N-(8-fluoro-6-oxo-1,2,3,4,5,6-hexahydrophenanthridin-1-yl)-N,4-dimethyl-1H-indole-2-carboxamide;
(S)-6-fluoro-N-(8-fluoro-6-oxo-1,2,3,4,5,6-hexahydrophenanthridin-1-yl)-N,4-dimethyl-1H-indole-2-carboxamide;
(R)-4-(difluoromethyl)-6-fluoro-N-(8-fluoro-6-oxo-1,2,3,4,5,6-hexahydrophenanthridin-1-yl)-N-methyl-1H-indole-2-carboxamide;
(S)-4-(difluoromethyl)-6-fluoro-N-(8-fluoro-6-oxo-1,2,3,4,5,6-hexahydrophenanthridin-1-yl)-N-methyl-1H-indole-2-carboxamide;
(S)-1-(((5,6-difluoro-1H-indol-2-yl)methyl)(methyl)amino)-8,9-difluoro-1,5-dihydro-2H-pyrano[3,4-c]isoquinolin-6(4H)-one;
(R)-1-(((5,6-difluoro-1H-indol-2-yl)methyl)(methyl)amino)-8,9-difluoro-1,5-dihydro-2H-pyrano[3,4-c]isoquinolin-6(4H)-one;
(S)-N-(8,9-difluoro-6-oxo-1,2,3,4,5,6-hexahydrobenzo[c][1,7]naphthyridin-1-yl)-N-methyl-1H-indazole-6-carboxamide;
(R)-N-(8,9-difluoro-6-oxo-1,2,3,4,5,6-hexahydrobenzo[c][1,7]naphthyridin-1-yl)-N-methyl-1H-indazole-6-carboxamide;
(S)-2-fluoro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-2-fluoro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-N-(8,9-difluoro-6-oxo-1,2,3,4,5,6-hexahydrobenzo[c][1,7]naphthyridin-1-yl)-2-fluoro-N-methyl-4H-thieno[3,2-b]pyrrole-5-carboxamide;
(R)-N-(8,9-difluoro-6-oxo-1,2,3,4,5,6-hexahydrobenzo[c][1,7]naphthyridin-1-yl)-2-fluoro-N-methyl-4H-thieno[3,2-b]pyrrole-5-carboxamide;
N-((1R)-8,9-difluoro-4-hydroxy-6-oxo-1,2,3,4,5,6-hexahydrophenanthridin-1-yl)-5-fluoro-N-methyl-1H-indole-2-carboxamide;
N-((1S)-8,9-difluoro-4-hydroxy-6-oxo-1,2,3,4,5,6-hexahydrophenanthridin-1-yl)-5-fluoro-N-methyl-1H-indole-2-carboxamide;
(R,R)-N-(8,9-difluoro-4-hydroxy-6-oxo-1,2,3,4,5,6-hexahydrophenanthridin-1-yl)-5-fluoro-N-methyl-1H-indole-2-carboxamide;
(S,R)-N-(8,9-difluoro-4-hydroxy-6-oxo-1,2,3,4,5,6-hexahydrophenanthridin-1-yl)-5-fluoro-N-methyl-1H-indole-2-carboxamide;
(S,S)-N-(8,9-difluoro-4-hydroxy-6-oxo-1,2,3,4,5,6-hexahydrophenanthridin-1-yl)-5-fluoro-N-methyl-1H-indole-2-carboxamide;
(R,S)-N-(8,9-difluoro-4-hydroxy-6-oxo-1,2,3,4,5,6-hexahydrophenanthridin-1-yl)-5-fluoro-N-methyl-1H-indole-2-carboxamide;
N-((1R)-8,9-difluoro-4-hydroxy-6-oxo-1,2,3,4,5,6-hexahydrophenanthridin-1-yl)-5,6-difluoro-N-methyl-1H-indole-2-carboxamide;
N-((1S)-8,9-difluoro-4-hydroxy-6-oxo-1,2,3,4,5,6-hexahydrophenanthridin-1-yl)-5,6-difluoro-N-methyl-1H-indole-2-carboxamide;
(S,R)-N-(8,9-difluoro-4-hydroxy-6-oxo-1,2,3,4,5,6-hexahydrophenanthridin-1-yl)-5,6-difluoro-N-methyl-1H-indole-2-carboxamide;
(R,R)-N-(8,9-difluoro-4-hydroxy-6-oxo-1,2,3,4,5,6-hexahydrophenanthridin-1-yl)-5,6-difluoro-N-methyl-1H-indole-2-carboxamide;
(R,S)-N-(8,9-difluoro-4-hydroxy-6-oxo-1,2,3,4,5,6-hexahydrophenanthridin-1-yl)-5,6-difluoro-N-methyl-1H-indole-2-carboxamide;
(S,S)-N-(8,9-difluoro-4-hydroxy-6-oxo-1,2,3,4,5,6-hexahydrophenanthridin-1-yl)-5,6-difluoro-N-methyl-1H-indole-2-carboxamide;
N-((1R)-8,9-difluoro-4-hydroxy-6-oxo-1,2,3,4,5,6-hexahydrophenanthridin-1-yl)-N-methyl-1H-indole-2-carboxamide;
N-((1S)-8,9-difluoro-4-hydroxy-6-oxo-1,2,3,4,5,6-hexahydrophenanthridin-1-yl)-N-methyl-1H-indole-2-carboxamide;
(S,R)-N-(8,9-difluoro-4-hydroxy-6-oxo-1,2,3,4,5,6-hexahydrophenanthridin-1-yl)-N-methyl-1H-indole-2-carboxamide;
(R,R)-N-(8,9-difluoro-4-hydroxy-6-oxo-1,2,3,4,5,6-hexahydrophenanthridin-1-yl)-N-methyl-1H-indole-2-carboxamide;
(R,S)-N-(8,9-difluoro-4-hydroxy-6-oxo-1,2,3,4,5,6-hexahydrophenanthridin-1-yl)-N-methyl-1H-indole-2-carboxamide;
(S,S)-N-(8,9-difluoro-4-hydroxy-6-oxo-1,2,3,4,5,6-hexahydrophenanthridin-1-yl)-N-methyl-1H-indole-2-carboxamide;
(R)-N-(8,9-difluoro-6-oxo-1,2,3,4,5,6-hexahydrobenzo[c][1,7]naphthyridin-1-yl)-N-methylindolizine-2-carboxamide;
(S)-N-(8,9-difluoro-6-oxo-1,2,3,4,5,6-hexahydrobenzo[c][1,7]naphthyridin-1-yl)-N-methylindolizine-2-carboxamide;
(R)-8-chloro-N-(8,9-difluoro-6-oxo-1,2,3,4,5,6-hexahydrobenzo[c][1,7]naphthyridin-1-yl)-N-methylindolizine-2-carboxamide;
(S)-8-chloro-N-(8,9-difluoro-6-oxo-1,2,3,4,5,6-hexahydrobenzo[c][1,7]naphthyridin-1-yl)-N-methylindolizine-2-carboxamide;
(S)-N-(8,9-Difluoro-6-oxo-1,4,5,6-tetrahydro-2H-pyrano[3,4-c]isoquinolin-1-yl)-2,2-difluoro-N-methyl-2-phenylacetamide;
(R)-N-(8,9-Difluoro-6-oxo-1,4,5,6-tetrahydro-2H-pyrano[3,4-c]isoquinolin-1-yl)-2,2-difluoro-N-methyl-2-phenylacetamide;
(S)-N-(8,9-Difluoro-6-oxo-1,4,5,6-tetrahydro-2H-pyrano[3,4-c]isoquinolin-1-yl-1-d)-5,6-difluoro-N-(methyl-13C-d3)-1H-indole-2-carboxamide;
(R)-N-(8,9-Difluoro-6-oxo-1,4,5,6-tetrahydro-2H-pyrano[3,4-c]isoquinolin-1-yl-1-d)-5,6-difluoro-N-(methyl-13C-d3)-1H-indole-2-carboxamide;
(S)-N-(8,9-Difluoro-6-oxo-1,4,5,6-tetrahydro-2H-pyrano[3,4-c]isoquinolin-1-yl-1-d)-6-(difluoromethyl)-5-fluoro-N-(methyl-13C-d3)-1H-indole-2-carboxamide;
(R)-N-(8,9-Difluoro-6-oxo-1,4,5,6-tetrahydro-2H-pyrano[3,4-c]isoquinolin-1-yl-1-d)-6-(difluoromethyl)-5-fluoro-N-(methyl-13C-d3)-1H-indole-2-carboxamide;
(S)-2-Chloro-N-(8,9-difluoro-6-oxo-1,4,5,6-tetrahydro-2H-pyrano[3,4-c]isoquinolin-1-yl-1-d)-N-(methyl-13C-d3)-4H-thieno[3,2-b]pyrrole-5-carboxamide; and
(R)-2-Chloro-N-(8,9-difluoro-6-oxo-1,4,5,6-tetrahydro-2H-pyrano[3,4-c]isoquinolin-1-yl-1-d)-N-(methyl-13C-d3)-4H-thieno[3,2-b]pyrrole-5-carboxamide;
   or a salt, solvate, prodrug, isotopically labelled derivative, stereoisomer, or tautomer thereof, or any mixtures thereof.

Embodiment 14 provides a pharmaceutical composition comprising at least one compound of any of Embodiments 1-13 and a pharmaceutically acceptable carrier.

Embodiment 15 provides the pharmaceutical composition of Embodiment 14, further comprising at least one additional agent useful for treating, ameliorating, and/or preventing hepatitis B virus infection.

Embodiment 16 provides the pharmaceutical composition of Embodiment 15, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

Embodiment 17 provides a method of treating, ameliorating, and/or preventing hepatitis B virus (HBV) infection in a subject, the method comprising administering to the subject in need thereof a therapeutically effective amount of at least one compound of any of Embodiments 1-13 and/or at least one pharmaceutical composition of any of Embodiments 14-16.

Embodiment 18 provides the method of Embodiment 17, wherein the subject is further infected with hepatitis D virus (HDV).

Embodiment 19 provides the method of any of Embodiments 17-18, wherein the at least one compound and/or composition is administered to the subject in a pharmaceutically acceptable composition.

Embodiment 20 provides the method of any of Embodiments 17-19, wherein the subject is further administered at least one additional agent useful for treating, ameliorating, and/or preventing the hepatitis B virus infection.

Embodiment 21 provides the method of Embodiment 20, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

Embodiment 22 provides the method of Embodiment 21, wherein the immunostimulator is a checkpoint inhibitor.

Embodiment 23 provides the method of Embodiment 22, wherein the checkpoint inhibitor is a PD-L1 inhibitor.

Embodiment 24 provides the method of any of Embodiments 20-23, wherein the subject is co-administered the at least one compound and/or composition and the at least one additional agent.

Embodiment 25 provides the method of any of Embodiments 20-24, wherein the at least one compound and/or composition and the at least one additional agent are coformulated.

Embodiment 26 provides a method of inhibiting expression and/or function of a viral capsid protein directly or indirectly in a hepatitis B virus-infected subject, the method comprising administering to the subject in need thereof a therapeutically effective amount of at least one compound of any of Embodiments 1-13 and/or at least one pharmaceutical composition of any of Embodiments 14-16.

Embodiment 27 provides the method of Embodiment 26, wherein the subject is further infected with hepatitis D virus (HDV).

Embodiment 28 provides the method of any of Embodiments 26-27, wherein the at least one compound and/or composition is administered to the subject in a pharmaceutically acceptable composition.

Embodiment 29 provides the method of any of Embodiments 26-28, wherein the subject is further administered at least one additional agent useful for treating, ameliorating, and/or preventing the hepatitis B viral infection.

Embodiment 30 provides the method of Embodiment 29, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

Embodiment 31 provides the method of Embodiment 30, wherein the immunostimulator is a checkpoint inhibitor.

Embodiment 32 provides the method of Embodiment 31, wherein the checkpoint inhibitor is a PD-L1 inhibitor.

Embodiment 33 provides the method of any of Embodiments 29-32, wherein the subject is co-administered the at least one compound and/or composition and the at least one additional agent.

Embodiment 34 provides the method of any of Embodiments 29-33, wherein the at least one compound and/or composition and the at least one additional agent are coformulated.

Embodiment 35 provides the method of any of Embodiments 17-34, wherein the subject is a mammal.

Embodiment 36 provides the method of Embodiment 35, wherein the mammal is a human.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound of formula (I), or a salt, solvate, prodrug, stereoisomer, tautomer, or isotopically labelled derivative thereof, or any mixtures thereof: 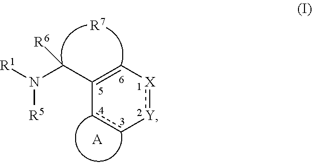
wherein:
X, Y, and the bond between X and Y are such that:
X is NR⁸, Y is C(═O), and the bond between X and Y is a single bond, or
X is N, Y is CR¹¹, and the bond between X and Y is a double bond;
ring A is selected from the group consisting of: 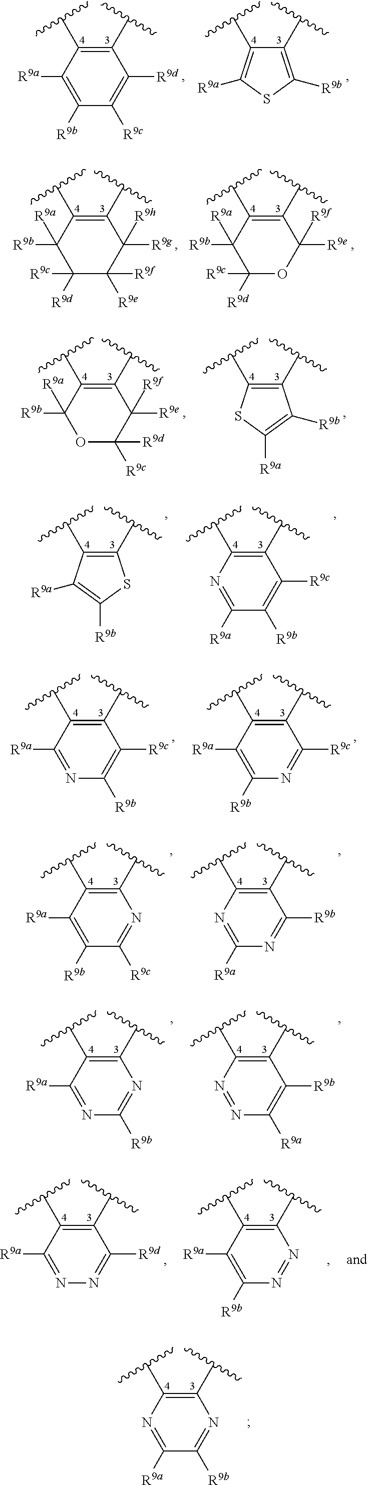
R¹ is selected from the group consisting of R²C(═O)—, R²S(═O)₂—, and aminoacyl;
R² is selected from the group consisting of: 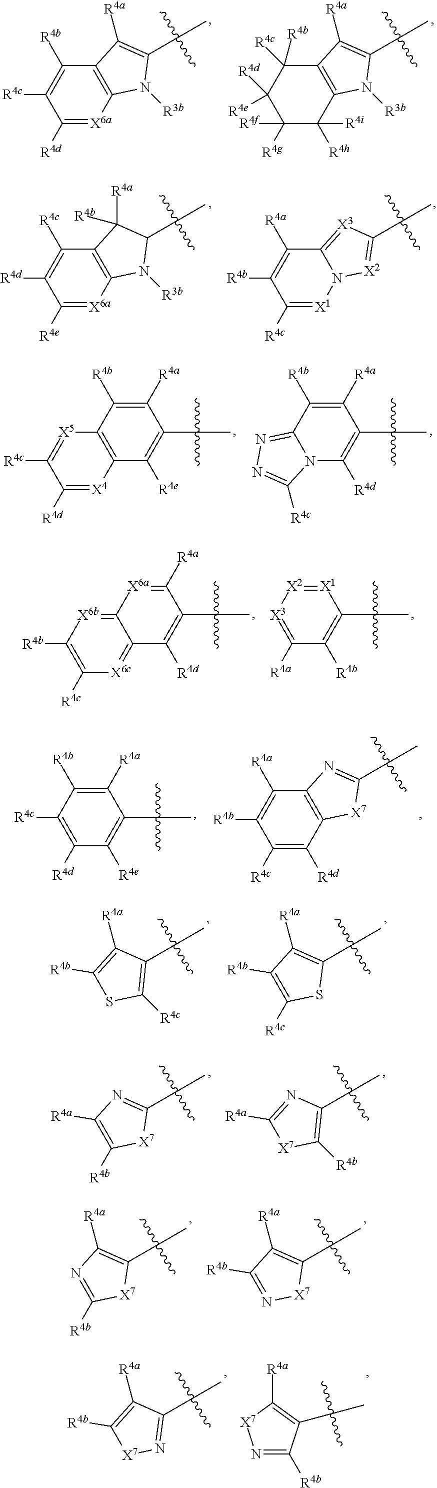 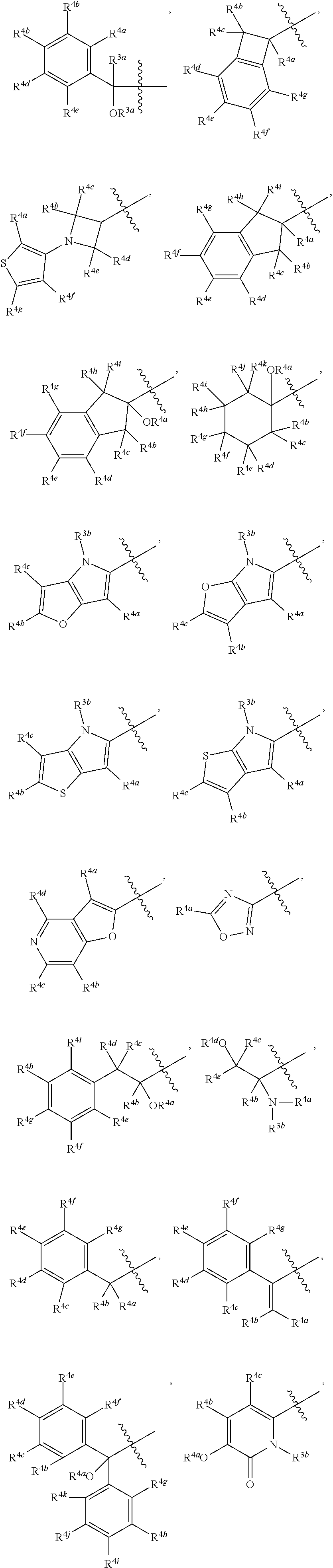 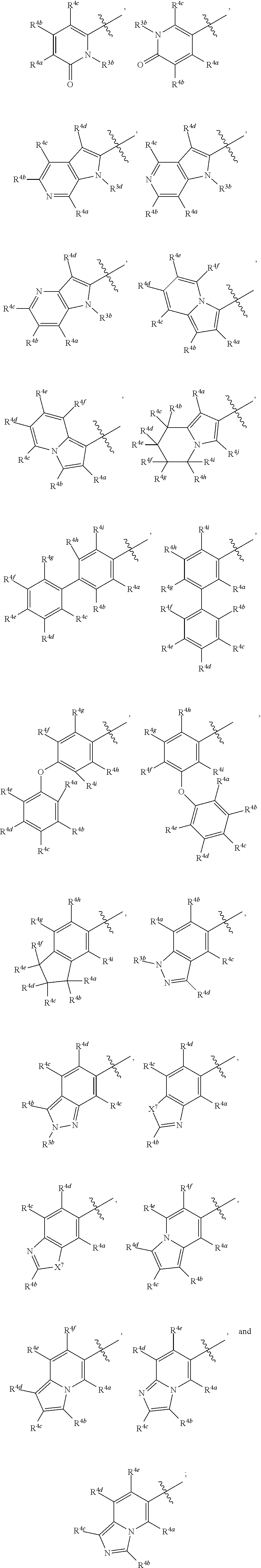
one of the following applies:
(i) X¹ is N, X² is C(R^{4f}), and X³ is C(R^{4g});
(ii) X² is N, X¹ is C(R^{4f}), and X³ is C(R^{4g});
(iii) X³ is N, X¹ is C(R^{4f}), and X² is C(R^{4g});
(v) X¹ is C(R^{4f}), X² is C(R^{4g}), and X³ is C(R^{4h});
one of the following applies:
(i) X⁴ is N and X⁵ is C(R^{4e}); or
(ii) X⁵ is N and X⁴ is C(R^{4e});
each occurrence of X^{6a} is independently N or C(R^{4f});
each occurrence of X^{6b} is independently N or C(R^{4g});
each occurrence of X^{6c} is independently N or C(R^{4h});
each occurrence of X⁷ is independently S, O, or NR^{3a};
each occurrence of R^{3a} is independently selected from the group consisting of H, optionally substituted C₁-C₆ alkyl, and optionally substituted C₃-C₈ cycloalkyl;
each occurrence of R^{3b} is independently selected from the group consisting of H, optionally substituted C₁-C₆ alkyl, and optionally substituted C₃-C₈ cycloalkyl;
each occurrence of R^{4a}, R^{4b}, R^{4c}, R^{4d}, R^{4e}, R^{4f}, R^{4g}, R^{4h}, R⁴ⁱ, R^{4j} and R^{4k} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, optionally substituted phenyl, optionally substituted heterocyclyl, optionally substituted heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R⁵ is independently selected from the group consisting of H, C₁-C₆ alkyl, and C₃-C₈ cycloalkyl, wherein the alkyl or cycloalkyl is optionally substituted with at least one selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, optionally substituted phenyl, optionally substituted heteroaryl, optionally substituted heterocyclyl, —C(═O)OR¹⁰, —OC(═O)R¹⁰, —SR¹⁰, —S(═O)R¹⁰, —S(═O)₂R¹⁰, —S(═O)₂NR¹⁰R¹⁰, —N(R¹⁰)S(═O)₂R¹⁰, —N(R¹⁰)C(═O)R¹⁰, —C(═O)NR¹⁰R¹⁰, and —NR¹⁰R¹⁰;
each occurrence of R⁶ is independently selected from the group consisting of H and optionally substituted C₁-C₆ alkyl;
R⁷ is —(CH₂)ₚ-Q-(CH₂)_{q}—, wherein p and q are independently 0, 1, or 2, and Q is a bond (absent), —O—, —S—, —S(O)—, —S(O)₂—, —NR¹², —CH(OH)—, —C(═O)—, —C(═O)O—, or —OC(═O)—,
wherein 2≤(p+q)≤4 if Q is a bond,
wherein 1≤(p+q)≤3 if Q is —O—, S—, —S(O)—, —S(O)₂—, —NR¹², —CH(OH)—, or —C(═O)—,
wherein 0≤(p+q)≤2 if Q is —C(═O)O— or —OC(═O)—, and
wherein each CH₂ in R⁷ is optionally substituted with at least one substituent selected from the group consisting of methyl, OR¹³, or halogen;
each occurrence of R⁸ is independently selected from the group consisting of H, optionally substituted C₁-C₆ alkyl, and optionally substituted C₃-C₈ cycloalkyl;
each occurrence of R^{9a}, R^{9b}, R^{9c}, R^{9d}, R^{9e}, R^{9f}, R^{9g}, and R^{9h} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, optionally substituted phenyl, optionally substituted heterocyclyl, optionally substituted heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R¹⁰ is independently selected from the group consisting of H, optionally substituted C₁-C₆ alkyl, optionally substituted C₃-C₈ cycloalkyl, optionally substituted phenyl, and optionally substituted hetereoaryl;
each occurrence of R¹¹ is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, optionally substituted phenyl, optionally substituted heterocyclyl, optionally substituted heteroaryl, —S(optionally substituted C₁-C₆ alkyl), —SO(optionally substituted C₁-C₆ alkyl), —SO₂(optionally substituted C₁-C₆ alkyl), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R¹² is independently selected from the group consisting of H, optionally substituted C₁-C₆ alkyl, optionally substituted C₁-C₆ hydroxyalkyl, optionally substituted C₃-C₈ cycloalkyl, optionally substituted phenyl, optionally substituted heteroaryl, and optionally substituted C₁-C₆ acyl;
each occurrence of R¹³ is independently selected from the group consisting of H, optionally substituted C₁-C₆ alkyl, and —C(═O)C₁-C₆ alkyl.

2. The compound of claim 1, wherein at least one of the following applies:
each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —C(═O)OR^{b}, —OC(═O)R^{b}, —SR^{b}, —S(═O)R^{b}, —S(═O)₂R^{b}, N(R^{b})S(═O)₂R^{b}, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—;
each occurrence of alkyl, alkenyl, alkynyl, or cycloalkyl is independently optionally substituted with at least one substituent selected from the group consisting of C₁-C₆ alkyl, C₃-C₈ cycloalkyl, halo, cyano (—CN), —OR^{a}, optionally substituted phenyl, optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

3. (canceled)

4. The compound of claim 1, wherein R² is selected from the group consisting of

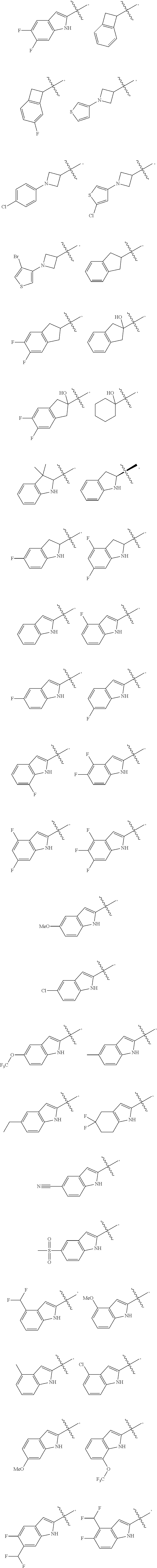

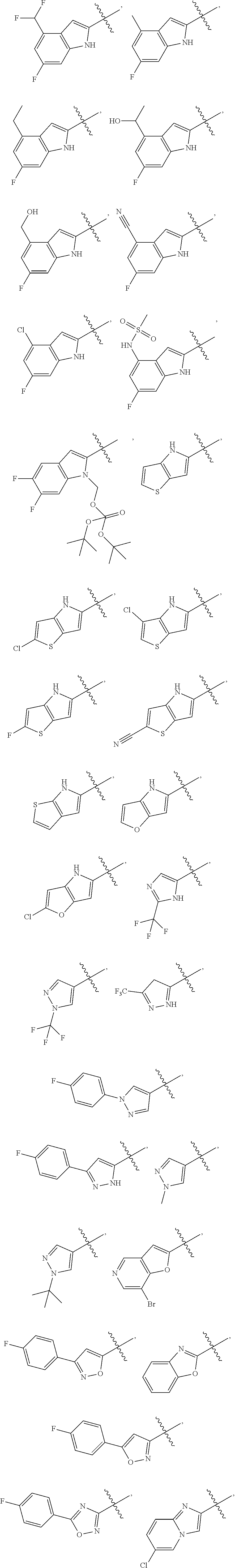

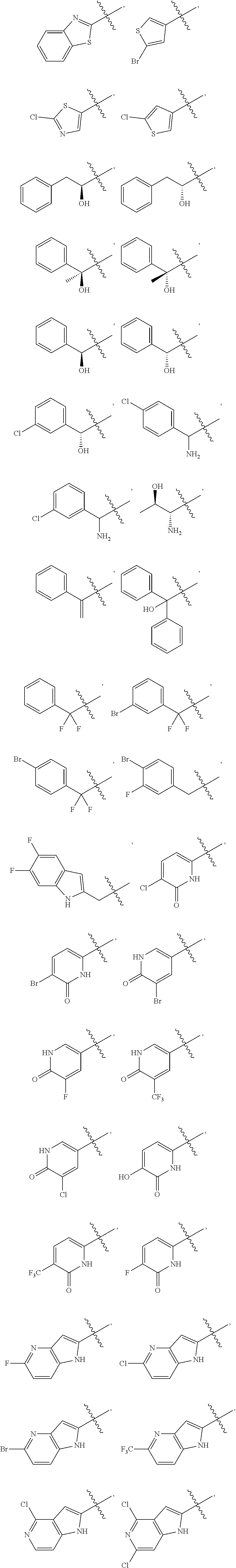

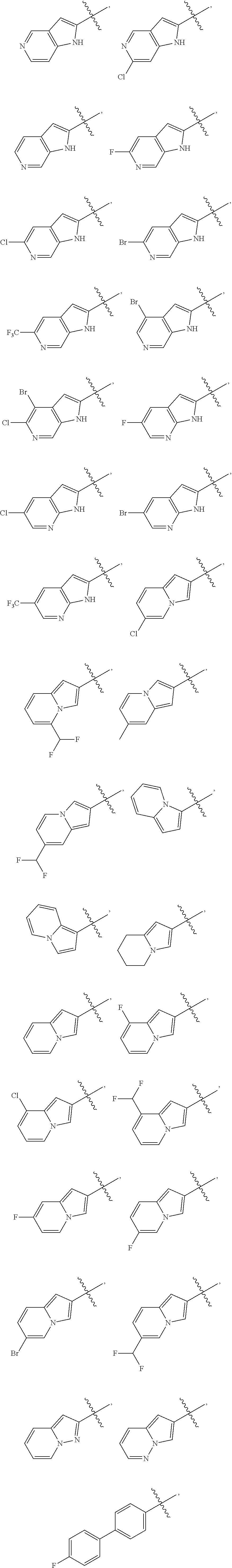

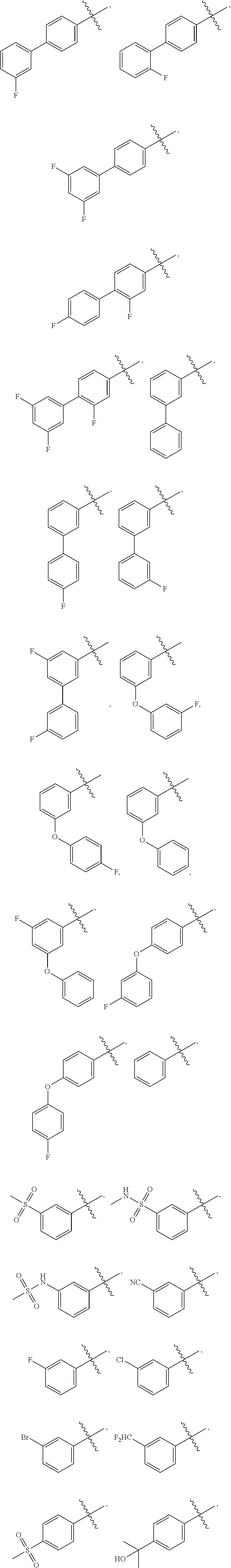

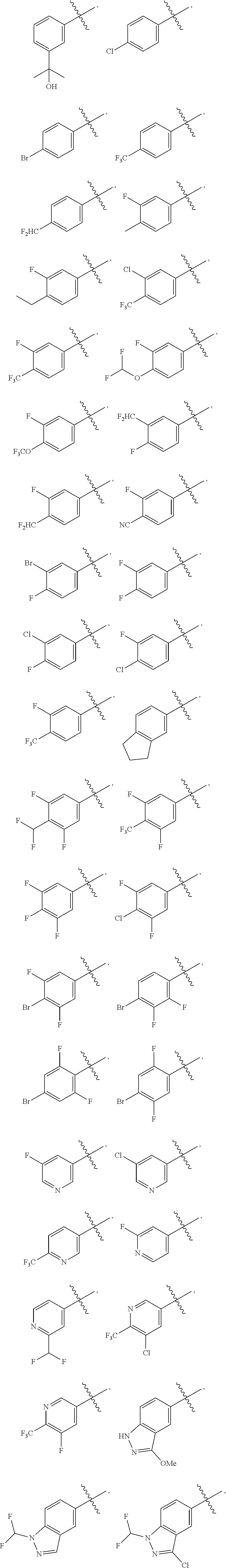

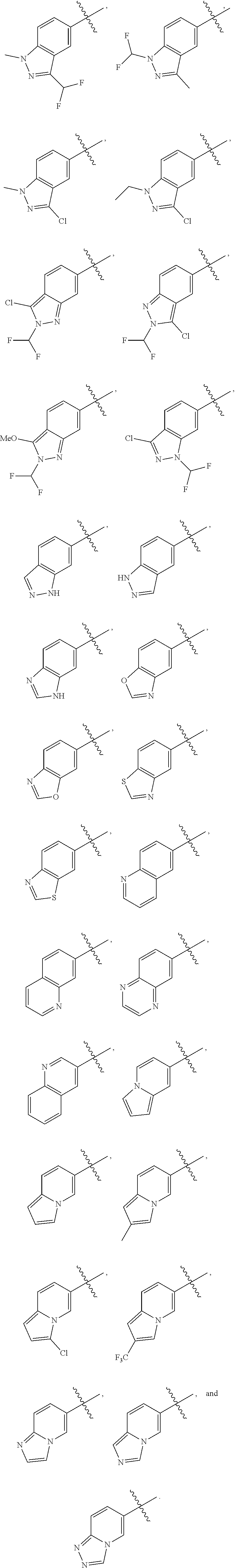

5. The compound of claim 1, wherein each of R^{3a} and R^{3b} is independently H or methyl.

6. The compound of claim 1, wherein R⁵ is selected from the group consisting of H, methyl, ethyl, isopropyl, n-propyl, cyclopropyl, n-butyl, isobutyl, sec-butyl, tert-butyl, cyclobutyl, isopropylmethyl, —(CH₂)₂₋₆OH, —(CH₂)₂₋₆O(C₁-C₆ alkyl), ¹³CD₃, optionally substituted benzyl, and optionally substituted phenyl.

7. The compound of claim 1, wherein R⁶ is selected from the group consisting of H, D, and CH₃.

8. The compound of claim 1, which is selected from the group consisting of:

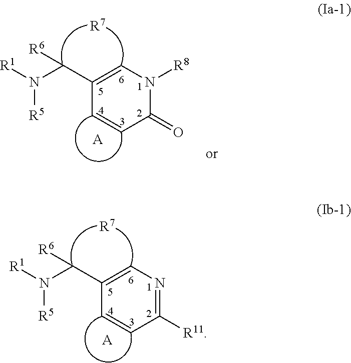

9. The compound of claim 1, which is selected from the group consisting of:

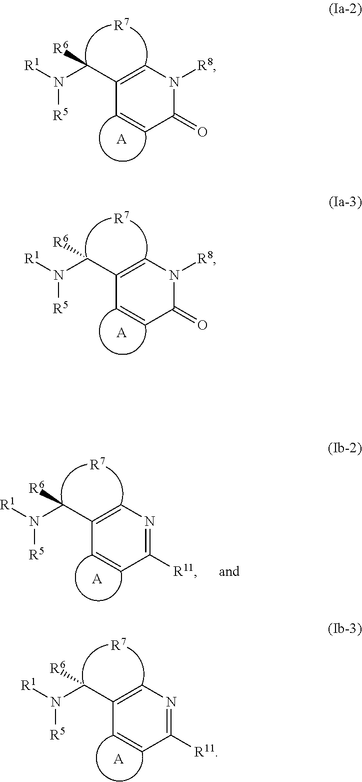

10. The compound of claim 1, which is selected from the group consisting of:

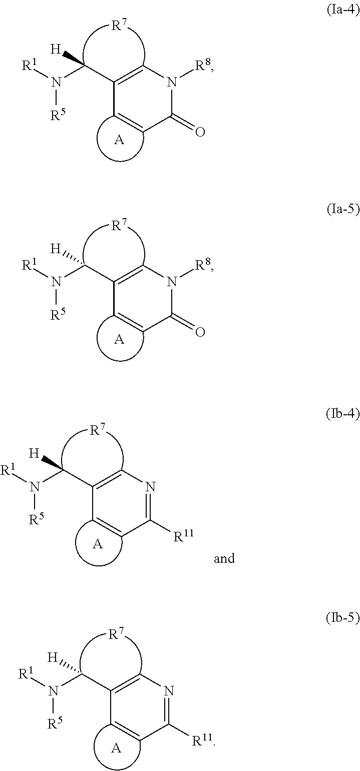

11. The compound of claim 1, which is selected from the group consisting of:

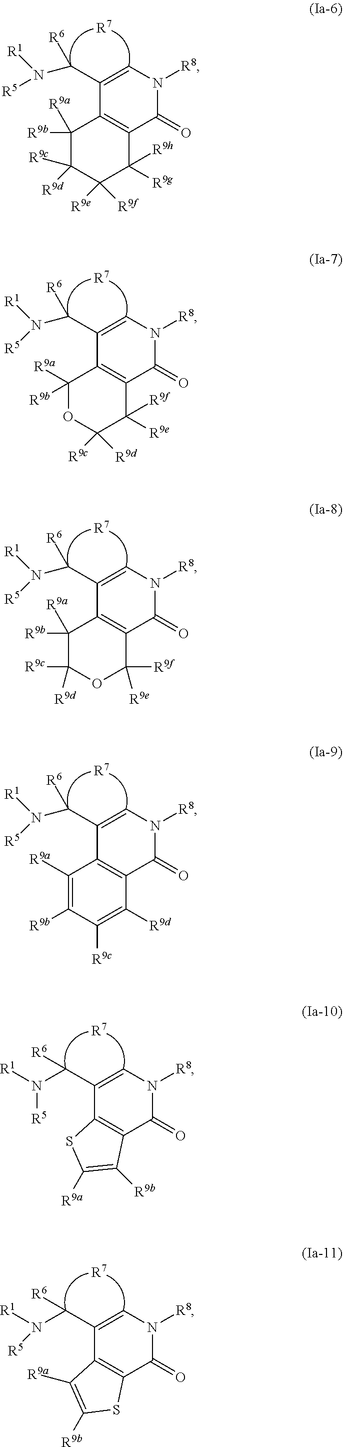

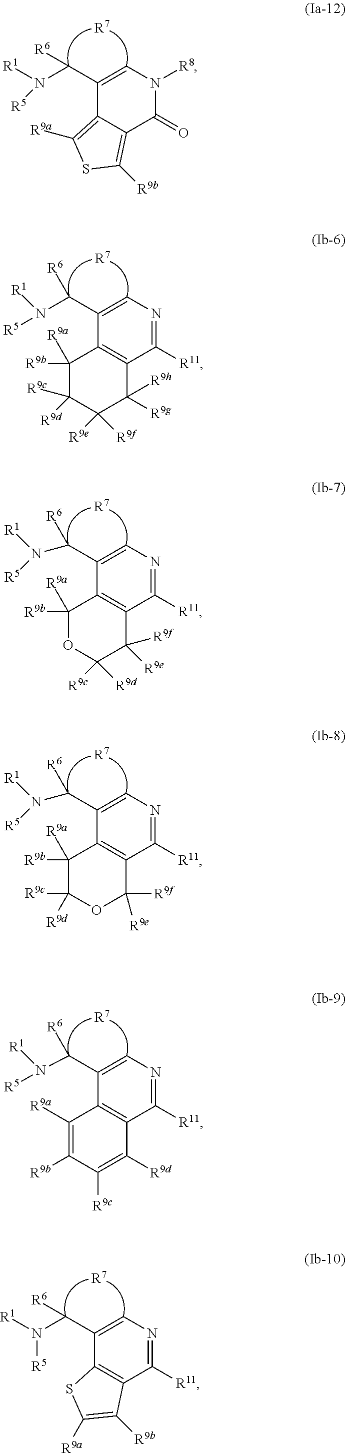

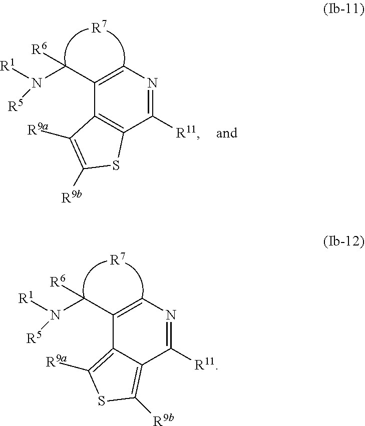

12. The compound of claim 1, which is at least one selected from the group consisting of:
N-(8-fluoro-6-oxo-1,4,5,6-tetrahydro-2H-pyrano[3,4-c]isoquinolin-1-yl)-N-methyl-1H-indole-2-carboxamide;
N-(8-fluoro-6-oxo-1,4,5,6-tetrahydro-2H-pyrano[3,4-c]isoquinolin-1-yl)-N-methylindoline-2-carboxamide;
N-(8,9-difluoro-6-oxo-1,4,5,6-tetrahydro-2H-pyrano[3,4-c]isoquinolin-1-yl)-5-fluoro-N-methylindoline-2-carboxamide;
N-(8,9-difluoro-6-oxo-1,4,5,6-tetrahydro-2H-pyrano[3,4-c]isoquinolin-1-yl)-8-fluoro-N-methylindolizine-2-carboxamide;
N-(8,9-difluoro-6-oxo-1,4,5,6-tetrahydro-2H-pyrano[3,4-c]isoquinolin-1-yl)-N-methylindolizine-2-carboxamide;
N-(8,9-difluoro-6-oxo-1,4,5,6-tetrahydro-2H-pyrano[3,4-c]isoquinolin-1-yl)-4,6-difluoro-N-methylindoline-2-carboxamide;
8-chloro-N-(8,9-difluoro-6-oxo-1,4,5,6-tetrahydro-2H-pyrano[3,4-c]isoquinolin-1-yl)-N-methylindolizine-2-carboxamide;
4-fluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-4-fluoro-N-methyl-1H-indole-2-carboxamide;
N-(8,9-difluoro-6-oxo-1,4,5,6-tetrahydro-2H-pyrano[3,4-c]isoquinolin-1-yl)-6-fluoro-N-methyl-1H-indole-2-carboxamide;
N-(8,9-difluoro-6-oxo-1,4,5,6-tetrahydro-2H-pyrano[3,4-c]isoquinolin-1-yl)-5-fluoro-N-methyl-1H-indole-2-carboxamide;
5-fluoro-N-(8-fluoro-6-oxo-1,4,5,6-tetrahydro-2H-pyrano[3,4-c]isoquinolin-1-yl)-N-methyl-1H-indole-2-carboxamide;
6-fluoro-N-(8-fluoro-6-oxo-1,4,5,6-tetrahydro-2H-pyrano[3,4-c]isoquinolin-1-yl)-N-methyl-1H-indole-2-carboxamide;
7-fluoro-N-(8-fluoro-6-oxo-1,4,5,6-tetrahydro-2H-pyrano[3,4-c]isoquinolin-1-yl)-N-methyl-1H-indole-2-carboxamide;
4,6-difluoro-N-(8-fluoro-6-oxo-1,4,5,6-tetrahydro-2H-pyrano[3,4-c]isoquinolin-1-yl)-N-methyl-1H-indole-2-carboxamide;
4,5-difluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-7-fluoro-N-methyl-1H-indole-2-carboxamide;
N-(8,9-difluoro-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-4,6-difluoro-N-methyl-1H-indole-2-carboxamide;
N-(8,9-difluoro-6-oxo-1,4,5,6-tetrahydro-2H-pyrano[3,4-c]isoquinolin-1-yl)-4,5-difluoro-N-methyl-1H-indole-2-carboxamide;
N-(8,9-difluoro-6-oxo-1,4,5,6-tetrahydro-2H-pyrano[3,4-c]isoquinolin-1-yl)-5,5-difluoro-N-methyl-4,5,6,7-tetrahydro-1H-indole-2-carboxamide;
5,6-difluoro-N-(8-fluoro-6-oxo-1,4,5,6-tetrahydro-2H-pyrano[3,4-c]isoquinolin-1-yl)-N-methyl-1H-indole-2-carboxamide;
6-chloro-N-(8,9-difluoro-6-oxo-1,4,5,6-tetrahydro-2H-pyrano[3,4-c]isoquinolin-1-yl)-N-methylimidazo[1,2-a]pyridine-2-carboxamide;
6-chloro-N-(8-fluoro-6-oxo-1,4,5,6-tetrahydro-2H-pyrano[3,4-c]isoquinolin-1-yl)-N-methylimidazo[1,2-a]pyridine-2-carboxamide;
6-chloro-N-(8-fluoro-6-oxo-1,4,5,6-tetrahydro-2H-pyrano[3,4-c]isoquinolin-1-yl)-N-ethylimidazo[1,2-a]pyridine-2-carboxamide;
N-(8,9-difluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N,3,3-trimethylindoline-2-carboxamide;
N-(8-fluoro-3-methyl-6-oxo-1,2,3,4,5,6-hexahydrobenzo[c][1,7]naphthyridin-1-yl)-N-methyl-1H-indole-2-carboxamide;
N-(8,9-difluoro-3-methyl-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methylpyrazolo[1,5-a]pyridine-2-carboxamide;
N-(8,9-difluoro-6-oxo-1,4,5,6-tetrahydro-2H-pyrano[3,4-c]isoquinolin-1-yl)-N-methylpyrrolo[1,2-b]pyridazine-6-carboxamide;
N-(8,9-difluoro-6-oxo-1,4,5,6-tetrahydro-2H-pyrano[3,4-c]isoquinolin-1-yl)-N-methylquinoline-7-carboxamide;
N-(8,9-difluoro-6-oxo-1,4,5,6-tetrahydro-2H-pyrano[3,4-c]isoquinolin-1-yl)-N-methylquinoline-6-carboxamide;
N-(3-Acetyl-8-fluoro-6-oxo-1,2,3,4,5,6-hexahydrobenzo[c][1,7]naphthyridin-1-yl)-N-methyl-1H-indole-2-carboxamide;
N-(8,9-Difluoro-6-oxo-1,4,5,6-tetrahydro-2H-pyrano[3,4-c]isoquinolin-1-yl)-N-methyl-[1,2,4]triazolo[4,3-a]pyridine-6-carboxamide;
N-(8,9-Difluoro-6-oxo-1,4,5,6-tetrahydro-2H-pyrano[3,4-c]isoquinolin-1-yl)-N-methylquinoline-3-carboxamide;
N-(8,9-Difluoro-6-oxo-1,4,5,6-tetrahydro-2H-pyrano[3,4-c]isoquinolin-1-yl)-N-methylquinoxaline-6-carboxamide;
N-(8,9-Difluoro-6-oxo-1,4,5,6-tetrahydro-2H-pyrano[3,4-c]isoquinolin-1-yl)-N-methyl-6-(trifluoromethyl)nicotinamide;
N-(8,9-Difluoro-6-oxo-1,4,5,6-tetrahydro-2H-pyrano[3,4-c]isoquinolin-1-yl)-3-fluoro-N-methyl-4-(trifluoromethyl)benzamide;
4-Chloro-N-(8,9-difluoro-6-oxo-1,4,5,6-tetrahydro-2H-pyrano[3,4-c]isoquinolin-1-yl)-3-fluoro-N-methylbenzamide;
N-(8,9-Difluoro-6-oxo-1,4,5,6-tetrahydro-2H-pyrano[3,4-c]isoquinolin-1-yl)-3,4,5-trifluoro-N-methylbenzamide;
N-(8-Fluoro-6-oxo-1,2,3,4,5,6-hexahydrobenzo[c][1,7]naphthyridin-1-yl)-N-methyl-1H-indole-2-carboxamide;
N-(8-Fluoro-3-(2-hydroxyethyl)-6-oxo-1,2,3,4,5,6-hexahydrobenzo[c][1,7]naphthyridin-1-yl)-N-methyl-1H-indole-2-carboxamide;
N-(3-Acetyl-8,9-difluoro-6-oxo-1,2,3,4,5,6-hexahydrobenzo[c][1,7]naphthyridin-1-yl)-N-methyl-1H-indole-2-carboxamide;
N-(8,9-Difluoro-6-oxo-1,4,5,6-tetrahydro-2H-pyrano[3,4-c]isoquinolin-1-yl)-5-fluoro-N-methylnicotinamide;
5-Chloro-N-(8,9-difluoro-6-oxo-1,4,5,6-tetrahydro-2H-pyrano[3,4-c]isoquinolin-1-yl)-N-methylnicotinamide;
N-(8,9-Difluoro-6-oxo-1,4,5,6-tetrahydro-2H-pyrano[3,4-c]isoquinolin-1-yl)-2-fluoro-N-methylisonicotinamide;
N-(8,9-Difluoro-6-oxo-1,4,5,6-tetrahydro-2H-pyrano[3,4-c]isoquinolin-1-yl)-3-(difluoromethyl)-N-methylbenzamide;
N-(8,9-Difluoro-6-oxo-1,4,5,6-tetrahydro-2H-pyrano[3,4-c]isoquinolin-1-yl)-2-hydroxy-N-methyl-3-phenylpropanamide;
N-(8,9-Difluoro-3-(2-hydroxyethyl)-6-oxo-1,2,3,4,5,6-hexahydrobenzo[c][1,7]naphthyridin-1-yl)-N-methyl-1H-indole-2-carboxamide;
N-(8,9-Difluoro-6-oxo-1,4,5,6-tetrahydro-2H-pyrano[3,4-c]isoquinolin-1-yl)-N-methylbenzamide;
N-(8,9-Difluoro-6-oxo-1,4,5,6-tetrahydro-2H-pyrano[3,4-c]isoquinolin-1-yl)-3,4-difluoro-N-methylbenzamide;
N-(8,9-Difluoro-6-oxo-1,4,5,6-tetrahydro-2H-pyrano[3,4-c]isoquinolin-1-yl)-2-hydroxy-N-methyl-2-phenylpropanamide;
N-(8,9-Difluoro-6-oxo-1,4,5,6-tetrahydro-2H-pyrano[3,4-c]isoquinolin-1-yl)-2-hydroxy-N-methyl-2-phenylacetamide;
N-(8,9-Difluoro-6-oxo-1,4,5,6-tetrahydro-2H-pyrano[3,4-c]isoquinolin-1-yl)-4,5,6-trifluoro-N-methyl-1H-indole-2-carboxamide;
N-(8,9-Difluoro-6-oxo-1,4,5,6-tetrahydro-2H-pyrano[3,4-c]isoquinolin-1-yl)-N-methyl-4-(trifluoromethyl)benzamide;
4-Chloro-N-(8,9-difluoro-6-oxo-1,4,5,6-tetrahydro-2H-pyrano[3,4-c]isoquinolin-1-yl)-N-methylbenzamide;
4-Cyano-N-(8,9-difluoro-6-oxo-1,4,5,6-tetrahydro-2H-pyrano[3,4-c]isoquinolin-1-yl)-3-fluoro-N-methylbenzamide;
N-(8,9-Difluoro-6-oxo-1,2,3,4,5,6-hexahydrobenzo[c][1,7]naphthyridin-1-yl)-5-fluoro-N-methyl-1H-indole-2-carboxamide;
N-(8,9-Difluoro-6-oxo-1,4,5,6-tetrahydro-2H-thiopyrano[3,4-c]isoquinolin-1-yl)-8-fluoro-N-methylindolizine-2-carboxamide;
N-(8,9-Difluoro-6-oxo-1,2,3,4,5,6-hexahydrobenzo[c][1,7]naphthyridin-1-yl)-4-fluoro-N-methyl-1H-indole-2-carboxamide;
N-(8,9-Difluoro-6-oxo-1,2,3,4,5,6-hexahydrobenzo[c][1,7]naphthyridin-1-yl)-6-fluoro-N-methyl-1H-indole-2-carboxamide;
N-(8,9-Difluoro-6-oxo-1,4,5,6-tetrahydro-2H-pyrano[3,4-c]isoquinolin-1-yl)-3-(difluoromethyl)-4-fluoro-N-methylbenzamide;
N-(8,9-Difluoro-6-oxo-1,4,5,6-tetrahydro-2H-pyrano[3,4-c]isoquinolin-1-yl)-4-(difluoromethyl)-3-fluoro-N-methylbenzamide;
N-(8,9-Difluoro-6-oxo-1,4,5,6-tetrahydro-2H-pyrano[3,4-c]isoquinolin-1-yl)-4-(difluoromethyl)-N-methylbenzamide;
3-Chloro-N-(8,9-difluoro-6-oxo-1,4,5,6-tetrahydro-2H-pyrano[3,4-c]isoquinolin-1-yl)-4-fluoro-N-methylbenzamide;
N-(8,9-Difluoro-6-oxo-1,2,3,4,5,6-hexahydrobenzo[c][1,7]naphthyridin-1-yl)-7-fluoro-N-methyl-1H-indole-2-carboxamide;
N-(8,9-Difluoro-6-oxo-1,2,3,4,5,6-hexahydrobenzo[c][1,7]naphthyridin-1-yl)-5,6-difluoro-N-methyl-1H-indole-2-carboxamide;
N-(8,9-Difluoro-6-oxo-1,2,3,4,5,6-hexahydrobenzo[c][1,7]naphthyridin-1-yl)-4,6-difluoro-N-methyl-1H-indole-2-carboxamide;
N-(8,9-Difluoro-6-oxo-1,2,3,4,5,6-hexahydrobenzo[c][1,7]naphthyridin-1-yl)-4,5-difluoro-N-methyl-1H-indole-2-carboxamide;
6-Chloro-N-(8,9-difluoro-6-oxo-1,2,3,4,5,6-hexahydrobenzo[c][1,7]naphthyridin-1-yl)-N-methylindolizine-2-carboxamide;
N-(8,9-Difluoro-6-oxo-1,4,5,6-tetrahydro-2H-pyrano[3,4-c]isoquinolin-1-yl)-N-methylbenzo[d]thiazole-2-carboxamide;
N-(8,9-Difluoro-6-oxo-1,4,5,6-tetrahydro-2H-pyrano[3,4-c]isoquinolin-1-yl)-N-methylbenzo[d]oxazole-2-carboxamide;
N-(8,9-Difluoro-6-oxo-1,4,5,6-tetrahydro-2H-pyrano[3,4-c]isoquinolin-1-yl)-N-methyl-3-(trifluoromethyl)-1H-pyrazole-5-carboxamide;
N-(8,9-Difluoro-6-oxo-1,4,5,6-tetrahydro-2H-pyrano[3,4-c]isoquinolin-1-yl)-3-(4-fluorophenyl)-N-methyl-1H-pyrazole-5-carboxamide;
N-(8,9-Difluoro-6-oxo-1,4,5,6-tetrahydro-2H-pyrano[3,4-c]isoquinolin-1-yl)-3-fluoro-N-methylbenzamide;
3-Chloro-N-(8,9-difluoro-6-oxo-1,4,5,6-tetrahydro-2H-pyrano[3,4-c]isoquinolin-1-yl)-N-methylbenzamide;
3-Bromo-N-(8,9-difluoro-6-oxo-1,4,5,6-tetrahydro-2H-pyrano[3,4-c]isoquinolin-1-yl)-N-methylbenzamide;
3-Bromo-N-(8,9-difluoro-6-oxo-1,4,5,6-tetrahydro-2H-pyrano[3,4-c]isoquinolin-1-yl)-4-fluoro-N-methylbenzamide;
4-Bromo-N-(8,9-difluoro-6-oxo-1,4,5,6-tetrahydro-2H-pyrano[3,4-c]isoquinolin-1-yl)-N-methylbenzamide;
3-Fluoro-N-methyl-N-(6-oxo-1,4,5,6-tetrahydro-2H-pyrano[3,4-c]isoquinolin-1-yl)-4-(trifluoromethyl)benzamide;
3-Fluoro-N-methyl-N-(4-oxo-4,5,8,9-tetrahydro-6H-pyrano[3,4-b]thieno[3,4-d]pyridin-9-yl)-4-(trifluoromethyl)benzamide;
N-(8-Cyano-6-oxo-1,4,5,6-tetrahydro-2H-pyrano[3,4-c]isoquinolin-1-yl)-3-fluoro-N-methyl-4-(trifluoromethyl)benzamide;
3-Cyano-N-(8,9-difluoro-6-oxo-1,4,5,6-tetrahydro-2H-pyrano[3,4-c]isoquinolin-1-yl)-N-methylbenzamide;
N-(8,9-Difluoro-6-oxo-1,4,5,6-tetrahydro-2H-pyrano[3,4-c]isoquinolin-1-yl)-3-fluoro-N-methyl-4-(trifluoromethoxy)benzamide;
N-(8,9-Difluoro-6-oxo-1,4,5,6-tetrahydro-2H-pyrano[3,4-c]isoquinolin-1-yl)-3-fluoro-N,4-dimethylbenzamide;
2-(3-Chlorophenyl)-N-(8,9-difluoro-6-oxo-1,4,5,6-tetrahydro-2H-pyrano[3,4-c]isoquinolin-1-yl)-2-hydroxy-N-methylacetamide;
N-(8,9-Difluoro-6-oxo-1,4,5,6-tetrahydro-2H-pyrano[3,4-c]isoquinolin-1-yl)-3,5-difluoro-N-methyl-4-(trifluoromethyl)benzamide;
5-Chloro-N-(8,9-difluoro-6-oxo-1,4,5,6-tetrahydro-2H-pyrano[3,4-c]isoquinolin-1-yl)-N-methylthiophene-3-carboxamide;
3-Chloro-N-(8,9-difluoro-6-oxo-1,4,5,6-tetrahydro-2H-pyrano[3,4-c]isoquinolin-1-yl)-4-fluoro-N-methylbenzenesulfonamide;
3-Chloro-N-(8,9-difluoro-6-oxo-1,4,5,6-tetrahydro-2H-pyrano[3,4-c]isoquinolin-1-yl)-N-methylbenzenesulfonamide;
3-Cyano-N-(8,9-difluoro-6-oxo-1,4,5,6-tetrahydro-2H-pyrano[3,4-c]isoquinolin-1-yl)-N-methylbenzenesulfonamide;
3-Cyano-N-(8,9-difluoro-6-oxo-1,4,5,6-tetrahydro-2H-pyrano[3,4-c]isoquinolin-1-yl)-4-fluoro-N-methylbenzenesulfonamide;
N-(8,9-Difluoro-6-oxo-1,4,5,6-tetrahydro-2H-pyrano[3,4-c]isoquinolin-1-yl)-4-ethyl-3-fluoro-N-methylbenzamide;
N-(8,9-difluoro-6-oxo-1,4,5,6-tetrahydro-2H-pyrano[3,4-c]isoquinolin-1-yl)indolizine-2-carboxamide;
N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
N-(8,9-difluoro-3,3-dioxido-6-oxo-1,4,5,6-tetrahydro-2H-thiopyrano[3,4-c]isoquinolin-1-yl)-8-fluoro-N-methylindolizine-2-carboxamide;
N-(8,9-difluoro-6-oxo-1,4,5,6-tetrahydro-2H-pyrano[3,4-c]isoquinolin-1-yl)-4-(difluoromethoxy)-3-fluoro-N-methylbenzamide;
N-(8,9-difluoro-6-oxo-1,4,5,6-tetrahydro-2H-pyrano[3,4-c]isoquinolin-1-yl)-4-(2-hydroxypropan-2-yl)-N-methylbenzamide;
N-(8,9-difluoro-6-oxo-1,4,5,6-tetrahydro-2H-pyrano[3,4-c]isoquinolin-1-yl)-3-(2-hydroxypropan-2-yl)-N-methylbenzamide;
N-(8,9-difluoro-6-oxo-1,4,5,6-tetrahydro-2H-pyrano[3,4-c]isoquinolin-1-yl)-5,6-difluoro-1H-indole-2-carboxamide;
N-(8,9-difluoro-6-oxo-1,4,5,6-tetrahydro-2H-pyrano[3,4-c]isoquinolin-1-yl)-4,6-difluoro-1H-indole-2-carboxamide;
N-(8,9-difluoro-6-oxo-1,4,5,6-tetrahydro-2H-pyrano[3,4-c]isoquinolin-1-yl)-3-fluoro-4-(trifluoromethyl)benzamide;
4-chloro-N-(8,9-difluoro-6-oxo-1,4,5,6-tetrahydro-2H-pyrano[3,4-c]isoquinolin-1-yl)-3-fluorobenzamide;
N-(8,9-difluoro-6-oxo-1,2,3,4,5,6-hexahydrobenzo[c][1,7]naphthyridin-1-yl)-8-fluoro-N-methylindolizine-2-carboxamide;
N-(8,9-difluoro-6-oxo-1,2,3,4,5,6-hexahydrobenzo[c][1,7]naphthyridin-1-yl)-7-fluoro-N-methylindolizine-2-carboxamide;
4-bromo-N-(8,9-difluoro-6-oxo-1,2,3,4,5,6-hexahydrobenzo[c][1,7]naphthyridin-1-yl)-3,5-difluoro-N-methylbenzamide;
4-bromo-3,5-difluoro-N-(8-fluoro-6-oxo-1,4,5,6-tetrahydro-2H-pyrano[3,4-c]isoquinolin-1-yl)-N-methylbenzamide;
4-chloro-N-(8,9-difluoro-6-oxo-1,2,3,4,5,6-hexahydrobenzo[c][1,7]naphthyridin-1-yl)-3,5-difluoro-N-methylbenzamide;
4-bromo-3,5-difluoro-N-(8-fluoro-6-oxo-1,2,3,4,5,6-hexahydrobenzo[c][1,7]naphthyridin-1-yl)-N-methylbenzamide;
4-bromo-N-(8,9-difluoro-6-oxo-1,4,5,6-tetrahydro-2H-pyrano[3,4-c]isoquinolin-1-yl)-3,5-difluoro-N-methylbenzamide;
4-chloro-N-(8,9-difluoro-6-oxo-1,4,5,6-tetrahydro-2H-pyrano[3,4-c]isoquinolin-1-yl)-3,5-difluoro-N-methylbenzamide;
4-chloro-3,5-difluoro-N-(8-fluoro-6-oxo-1,4,5,6-tetrahydro-2H-pyrano[3,4-c]isoquinolin-1-yl)-N-methylbenzamide;
4-chloro-3,5-difluoro-N-(8-fluoro-6-oxo-1,2,3,4,5,6-hexahydrobenzo[c][1,7]naphthyridin-1-yl)-N-methylbenzamide;
4-(difluoromethyl)-3,5-difluoro-N-(8-fluoro-6-oxo-1,4,5,6-tetrahydro-2H-pyrano[3,4-c]isoquinolin-1-yl)-N-methylbenzamide;
N-(8,9-difluoro-6-oxo-1,4,5,6-tetrahydro-2H-pyrano[3,4-c]isoquinolin-1-yl)-4-(difluoromethyl)-3,5-difluoro-N-methylbenzamide;
4-(difluoromethyl)-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,2,3,4,5,6-hexahydrobenzo[c][1,7]naphthyridin-1-yl)-4-(difluoromethyl)-3,5-difluoro-N-methylbenzamide;
4-(difluoromethyl)-3,5-difluoro-N-(8-fluoro-6-oxo-1,2,3,4,5,6-hexahydrobenzo[c][1,7]naphthyridin-1-yl)-N-methylbenzamide;
N-(8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-N,1-dimethyl-1H-pyrazole-4-carboxamide;
N-(8,9-difluoro-6-oxo-1,4,5,6-tetrahydro-2H-pyrano[3,4-c]isoquinolin-1-yl)-N-methyl-2-phenylacrylamide;
N-(8,9-difluoro-6-oxo-1,4,5,6-tetrahydro-2H-pyrano[3,4-c]isoquinolin-1-yl)-5-fluoro-N-methyl-1H-pyrrolo[2,3-b]pyridine-2-carboxamide;
5-chloro-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
5-bromo-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-3-carboxamide;
5-bromo-N-(8,9-difluoro-6-oxo-1,4,5,6-tetrahydro-2H-pyrano[3,4-c]isoquinolin-1-yl)-N-methylthiophene-3-carboxamide;
N-(8,9-difluoro-6-oxo-1,4,5,6-tetrahydro-2H-pyrano[3,4-c]isoquinolin-1-yl)-N-methyl-2,3-dihydro-1H-indene-2-carboxamide;
1-(tert-butyl)-N-(8,9-difluoro-6-oxo-1,4,5,6-tetrahydro-2H-pyrano[3,4-c]isoquinolin-1-yl)-N-methyl-1H-pyrazole-4-carboxamide;
N-(8,9-difluoro-6-oxo-1,4,5,6-tetrahydro-2H-pyrano[3,4-c]isoquinolin-1-yl)-N-methyl-1-(trifluoromethyl)-1H-pyrazole-4-carboxamide;
4-bromo-N-(8,9-difluoro-6-oxo-1,4,5,6-tetrahydro-2H-pyrano[3,4-c]isoquinolin-1-yl)-N-methyl-1H-pyrrole-2-carboxamide;
2-amino-2-(4-chlorophenyl)-N-(8,9-difluoro-6-oxo-1,4,5,6-tetrahydro-2H-pyrano[3,4-c]isoquinolin-1-yl)-N-methylacetamide;
5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
2-amino-2-(3-chlorophenyl)-N-(8,9-difluoro-6-oxo-1,4,5,6-tetrahydro-2H-pyrano[3,4-c]isoquinolin-1-yl)-N-methylacetamide;
5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
5-chloro-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
N-(8,9-difluoro-6-oxo-1,2,3,4,5,6-hexahydrobenzo[c][1,7]naphthyridin-1-yl)-4-(difluoromethyl)-N-methyl-1H-indole-2-carboxamide;
4-(difluoromethyl)-N-(8-fluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-1-hydroxy-N-methylcyclohexane-1-carboxamide;
N-(8,9-difluoro-6-oxo-1,4,5,6-tetrahydro-2H-pyrano[3,4-c]isoquinolin-1-yl)-6-(difluoromethyl)-5-fluoro-N-methyl-1H-indole-2-carboxamide;
5-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-b]pyridine-2-carboxamide;
5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
6-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methyl-5-(trifluoromethyl)-1H-pyrrolo[3,2-b]pyridine-2-carboxamide;
N-(8,9-difluoro-6-oxo-1,2,3,4,5,6-hexahydrobenzo[c][1,7]naphthyridin-1-yl)-6-(difluoromethyl)-5-fluoro-N-methyl-1H-indole-2-carboxamide;
N-(8,9-difluoro-6-oxo-1,2,3,4,5,6-hexahydrobenzo[c][1,7]naphthyridin-1-yl)-6-fluoro-N-methylindolizine-2-carboxamide;
N-(8,9-difluoro-6-oxo-1,4,5,6-tetrahydro-2H-pyrano[3,4-c]isoquinolin-1-yl)-6-fluoro-N-methylindolizine-2-carboxamide;
N-(8,9-difluoro-6-oxo-1,4,5,6-tetrahydro-2H-pyrano[3,4-c]isoquinolin-1-yl)-N-methyl-2-(trifluoromethyl)-1H-imidazole-5-carboxamide;
N-(8,9-difluoro-6-oxo-1,4,5,6-tetrahydro-2H-pyrano[3,4-c]isoquinolin-1-yl)-5-fluoro-N-methyl-6-oxo-1,6-dihydropyridine-3-carboxamide;
N-(8,9-difluoro-6-oxo-1,4,5,6-tetrahydro-2H-pyrano[3,4-c]isoquinolin-1-yl)-N-methylindolizine-6-carboxamide;
N-(8,9-difluoro-6-oxo-1,4,5,6-tetrahydro-2H-pyrano[3,4-c]isoquinolin-1-yl)-N-methylindolizine-7-carboxamide;
N-(8,9-difluoro-6-oxo-1,4,5,6-tetrahydro-2H-pyrano[3,4-c]isoquinolin-1-yl)-2-hydroxy-N-methyl-2,2-diphenylacetamide;
N-(8,9-difluoro-6-oxo-1,4,5,6-tetrahydro-2H-pyrano[3,4-c]isoquinolin-1-yl)-4-(difluoromethyl)-6-fluoro-N-methyl-1H-indole-2-carboxamide;
N-(8,9-difluoro-6-oxo-1,4,5,6-tetrahydro-2H-pyrano[3,4-c]isoquinolin-1-yl)-7-fluoro-N-methylindolizine-2-carboxamide;
N-(8,9-difluoro-6-oxo-1,4,5,6-tetrahydro-2H-pyrano[3,4-c]isoquinolin-1-yl)-5-fluoro-N-methyl-1H-pyrrolo[3,2-b]pyridine-2-carboxamide;
N-(8,9-difluoro-6-oxo-1,4,5,6-tetrahydro-2H-pyrano[3,4-c]isoquinolin-1-yl)-5,6-difluoro-N-methyl-2,3-dihydro-1H-indene-2-carboxamide;
N-(8,9-difluoro-6-oxo-1,4,5,6-tetrahydro-2H-pyrano[3,4-c]isoquinolin-1-yl)-1-(4-fluorophenyl)-N-methyl-1H-pyrazole-4-carboxamide;
N-(8,9-difluoro-6-oxo-1,4,5,6-tetrahydro-2H-pyrano[3,4-c]isoquinolin-1-yl)-3-(4-fluorophenyl)-N-methylisoxazole-5-carboxamide;
N-(8,9-difluoro-6-oxo-1,4,5,6-tetrahydro-2H-pyrano[3,4-c]isoquinolin-1-yl)-4-(3-fluorophenoxy)-N-methylbenzamide;
4-(difluoromethyl)-6-fluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-5-methyl-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-6-methoxy-1,4-dihydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-N-methyl-5,6,7,8-tetrahydroindolizine-2-carboxamide;
4-bromo-N-(8,9-difluoro-6-oxo-1,4,5,6-tetrahydro-2H-pyrano[3,4-c]isoquinolin-1-yl)-2,3-difluoro-N-methylbenzamide;
4-bromo-N-(8,9-difluoro-6-oxo-1,4,5,6-tetrahydro-2H-pyrano[3,4-c]isoquinolin-1-yl)-2,5-difluoro-N-methylbenzamide;
4-bromo-N-(8,9-difluoro-6-oxo-1,4,5,6-tetrahydro-2H-pyrano[3,4-c]isoquinolin-1-yl)-2,6-difluoro-N-methylbenzamide;
N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-2-carboxamide;
N-(8,9-difluoro-6-oxo-1,4,5,6-tetrahydro-2H-pyrano[3,4-c]isoquinolin-1-yl)-N-methylindolizine-3-carboxamide;
N-(8,9-difluoro-6-oxo-1,4,5,6-tetrahydro-2H-pyrano[3,4-c]isoquinolin-1-yl)-4-(4-fluorophenoxy)-N-methylbenzamide;
N-(8,9-difluoro-6-oxo-1,4,5,6-tetrahydro-2H-pyrano[3,4-c]isoquinolin-1-yl)-4′-fluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-c]pyridine-2-carboxamide;
2-(3-bromophenyl)-N-(8,9-difluoro-6-oxo-1,4,5,6-tetrahydro-2H-pyrano[3,4-c]isoquinolin-1-yl)-2,2-difluoro-N-methylacetamide;
2-(4-bromophenyl)-N-(8,9-difluoro-6-oxo-1,4,5,6-tetrahydro-2H-pyrano[3,4-c]isoquinolin-1-yl)-2,2-difluoro-N-methylacetamide;
N-(8,9-difluoro-6-oxo-1,4,5,6-tetrahydro-2H-pyrano[3,4-c]isoquinolin-1-yl)-4-(difluoromethyl)-5-fluoro-N-methyl-1H-indole-2-carboxamide;
4-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,2,3,4,5,6-hexahydrobenzo[c][1,7]naphthyridin-1-yl)-4-(difluoromethyl)-6-fluoro-N-methyl-1H-indole-2-carboxamide;
4-(difluoromethyl)-6-fluoro-N-(8-fluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methylindolizine-1-carboxamide;
N-(8,9-difluoro-6-oxo-1,4,5,6-tetrahydro-2H-pyrano[3,4-c]isoquinolin-1-yl)-2,4′-difluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-5-(4-fluorophenyl)-N-methylisoxazole-3-carboxamide;
N-(8,9-difluoro-6-oxo-1,2,3,4,5,6-hexahydrobenzo[c][1,7]naphthyridin-1-yl)-4-(difluoromethyl)-5-fluoro-N-methyl-1H-indole-2-carboxamide;
N-(8,9-difluoro-6-oxo-1,4,5,6-tetrahydro-2H-pyrano[3,4-c]isoquinolin-1-yl)-5-(difluoromethyl)-N-methylindolizine-2-carboxamide;
5-bromo-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
N-(8,9-difluoro-6-oxo-1,4,5,6-tetrahydro-2H-pyrano[3,4-c]isoquinolin-1-yl)-N-methylbicyclo[4.2.0]octa-1(6),2,4-triene-7-carboxamide;
4-(difluoromethyl)-5-fluoro-N-(8-fluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-2-hydroxy-N-methyl-2,3-dihydro-1H-indene-2-carboxamide;
N-(8,9-difluoro-6-oxo-1,4,5,6-tetrahydro-2H-pyrano[3,4-c]isoquinolin-1-yl)-5,6-difluoro-2-hydroxy-N-methyl-2,3-dihydro-1H-indene-2-carboxamide;
N-(8,9-difluoro-6-oxo-1,4,5,6-tetrahydro-2H-pyrano[3,4-c]isoquinolin-1-yl)-N,2-dimethylindolizine-6-carboxamide;
N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
N-(8,9-difluoro-6-oxo-1,4,5,6-tetrahydro-2H-pyrano[3,4-c]isoquinolin-1-yl)-4-fluoro-N-methylbicyclo[4.2.0]octa-1(6),2,4-triene-7-carboxamide;
N-(6-(2-aminoethoxy)-8,9-difluoro-1,4-dihydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-N-methyl-1-(thiophen-3-yl)azetidine-3-carboxamide;
N-(8,9-difluoro-6-oxo-1,4,5,6-tetrahydro-2H-pyrano[3,4-c]isoquinolin-1-yl)-3′-fluoro-N-methyl-[1,1′-biphenyl]-4-carboxamide;
1-(4-bromothiophen-3-yl)-N-(8,9-difluoro-6-oxo-1,4,5,6-tetrahydro-2H-pyrano[3,4-c]isoquinolin-1-yl)-N-methylazetidine-3-carboxamide;
N-(8,9-difluoro-6-oxo-1,4,5,6-tetrahydro-2H-pyrano[3,4-c]isoquinolin-1-yl)-2′-fluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-3′,5′-difluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-2,3′,5′-trifluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-N-methyl-[1,1′-biphenyl]-3-carboxamide;
N-(8,9-difluoro-6-oxo-1,4,5,6-tetrahydro-2H-pyrano[3,4-c]isoquinolin-1-yl)-4′-fluoro-N-methyl-[1,1′-biphenyl]-3-carboxamide;
N-(8,9-difluoro-6-oxo-1,4,5,6-tetrahydro-2H-pyrano[3,4-c]isoquinolin-1-yl)-3′-fluoro-N-methyl-[1,1′-biphenyl]-3-carboxamide;
N-(8,9-difluoro-6-oxo-1,4,5,6-tetrahydro-2H-pyrano[3,4-c]isoquinolin-1-yl)-3′,5-difluoro-N-methyl-[1,1′-biphenyl]-3-carboxamide;
N-(8,9-difluoro-6-oxo-1,4,5,6-tetrahydro-2H-pyrano[3,4-c]isoquinolin-1-yl)-N-methyl-3-phenoxybenzamide;
N-(8,9-difluoro-6-oxo-1,4,5,6-tetrahydro-2H-pyrano[3,4-c]isoquinolin-1-yl)-3-(4-fluorophenoxy)-N-methylbenzamide;
N-(8,9-difluoro-6-oxo-1,4,5,6-tetrahydro-2H-pyrano[3,4-c]isoquinolin-1-yl)-N-methyl-6H-thieno[2,3-b]pyrrole-5-carboxamide;
N-(8,9-difluoro-6-oxo-1,4,5,6-tetrahydro-2H-pyrano[3,4-c]isoquinolin-1-yl)-N-methyl-4-(methylsulfonyl)benzamide;
N-(8,9-difluoro-6-oxo-1,4,5,6-tetrahydro-2H-pyrano[3,4-c]isoquinolin-1-yl)-N-methyl-3-(N-methylsulfamoyl)benzamide;
N-(8,9-difluoro-6-oxo-1,4,5,6-tetrahydro-2H-pyrano[3,4-c]isoquinolin-1-yl)-N-methyl-3-(methylsulfonamido)benzamide;
N-(8,9-difluoro-6-oxo-1,4,5,6-tetrahydro-2H-pyrano[3,4-c]isoquinolin-1-yl)-N-methyl-3-(methylsulfonyl)benzamide;
N-(8,9-difluoro-6-oxo-1,4,5,6-tetrahydro-2H-pyrano[3,4-c]isoquinolin-1-yl)-N-methylimidazo[1,5-a]pyridine-6-carboxamide;
1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
N-(8,9-difluoro-6-oxo-1,2,3,4,5,6-hexahydrobenzo[c][1,7]naphthyridin-1-yl)-3′-fluoro-N-methyl-[1,1′-biphenyl]-4-carboxamide;
N-(8,9-difluoro-6-oxo-1,4,5,6-tetrahydro-2H-pyrano[3,4-c]isoquinolin-1-yl)-3-fluoro-N-methyl-5-phenoxybenzamide;
N-(8,9-difluoro-6-oxo-1,2,3,4,5,6-hexahydrobenzo[c][1,7]naphthyridin-1-yl)-3′-fluoro-N-methyl-[1,1′-biphenyl]-3-carboxamide;
N-(8,9-difluoro-6-oxo-1,4,5,6-tetrahydro-2H-pyrano[3,4-c]isoquinolin-1-yl)-N-methylimidazo[1,2-a]pyridine-6-carboxamide;
N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
6-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
N-(8,9-difluoro-6-oxo-1,4,5,6-tetrahydro-2H-pyrano[3,4-c]isoquinolin-1-yl)-5-fluoro-N-methyl-1H-pyrrolo[2,3-c]pyridine-2-carboxamide;
N-(8,9-difluoro-6-oxo-1,4,5,6-tetrahydro-2H-pyrano[3,4-c]isoquinolin-1-yl)-N-methyl-2-(trifluoromethyl)indolizine-6-carboxamide;
4-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
N-(8,9-difluoro-6-oxo-1,4,5,6-tetrahydro-2H-pyrano[3,4-c]isoquinolin-1-yl)-2-(difluoromethyl)-N-methylisonicotinamide;
N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
N-(8,9-difluoro-5-(2-hydroxyethyl)-6-oxo-1,4,5,6-tetrahydro-2H-pyrano[3,4-c]isoquinolin-1-yl)-5,6-difluoro-N-methyl-1H-indole-2-carboxamide;
N-(5-(2-aminoethyl)-8,9-difluoro-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-6-(2-hydroxyethoxy)-1,4-dihydro-2H-pyrano[3,4-c]isoquinolin-1-yl)-5,6-difluoro-N-methyl-1H-indole-2-carboxamide;
N-(8,9-difluoro-6-oxo-1,2,3,4,5,6-hexahydrobenzo[c][1,7]naphthyridin-1-yl)-N-methyl-3-(N-methylsulfamoyl)benzamide;
N-(8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-(8,9-difluoro-4,6-dioxo-1,4,5,6-tetrahydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-3-(3-fluorophenoxy)-N-methylbenzamide;
N-(8,9-difluoro-6-oxo-1,4,5,6-tetrahydro-2H-pyrano[3,4-c]isoquinolin-1-yl)-5-methoxy-N-methyl-1H-indole-2-carboxamide;
5-chloro-N-(8,9-difluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N,5-dimethyl-1H-indole-2-carboxamide;
N-(8,9-difluoro-6-oxo-1,4,5,6-tetrahydro-2H-pyrano[3,4-c]isoquinolin-1-yl)-N-methyl-5-(trifluoromethoxy)-1H-indole-2-carboxamide;
N-(8,9-difluoro-6-oxo-1,4,5,6-tetrahydro-2H-pyrano[3,4-c]isoquinolin-1-yl)-5-ethyl-N-methyl-1H-indole-2-carboxamide;
N-(8,9-difluoro-6-oxo-1,4,5,6-tetrahydro-2H-pyrano[3,4-c]isoquinolin-1-yl)-4-methoxy-N-methyl-1H-indole-2-carboxamide;
N-(8,9-difluoro-6-oxo-1,4,5,6-tetrahydro-2H-pyrano[3,4-c]isoquinolin-1-yl)-N,4-dimethyl-1H-indole-2-carboxamide;
1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
2-chloro-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
N-(8,9-difluoro-6-(methylamino)-1,4-dihydro-2H-pyrano[3,4-c]isoquinolin-1-yl)-5,6-difluoro-N-methyl-1H-indole-2-carboxamide;
N-(8,9-difluoro-6-((2-hydroxyethyl)amino)-1,4-dihydro-2H-pyrano[3,4-c]isoquinolin-1-yl)-5,6-difluoro-N-methyl-1H-indole-2-carboxamide;
N-(8,9-difluoro-6-oxo-1,2,3,4,5,6-hexahydrobenzo[c][1,7]naphthyridin-1-yl)-3-(3-fluorophenoxy)-N-methylbenzamide;
4-cyano-N-(8,9-difluoro-6-oxo-1,4,5,6-tetrahydro-2H-pyrano[3,4-c]isoquinolin-1-yl)-6-fluoro-N-methyl-1H-indole-2-carboxamide;
N-(8,9-difluoro-6-oxo-1,4,5,6-tetrahydro-2H-pyrano[3,4-c]isoquinolin-1-yl)-4-ethyl-6-fluoro-N-methyl-1H-indole-2-carboxamide;
4-chloro-N-(8,9-difluoro-6-oxo-1,4,5,6-tetrahydro-2H-pyrano[3,4-c]isoquinolin-1-yl)-6-fluoro-N-methyl-1H-indole-2-carboxamide;
N-(8,9-difluoro-6-oxo-1,4,5,6-tetrahydro-2H-pyrano[3,4-c]isoquinolin-1-yl)-6-fluoro-N,4-dimethyl-1H-indole-2-carboxamide;
5-cyano-N-(8,9-difluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methyl-5-(methylsulfonyl)-1H-indole-2-carboxamide;
4-chloro-N-(8,9-difluoro-6-oxo-1,4,5,6-tetrahydro-2H-pyrano[3,4-c]isoquinolin-1-yl)-N-methyl-1H-indole-2-carboxamide;
6-bromo-N-(8,9-difluoro-6-oxo-1,4,5,6-tetrahydro-2H-pyrano[3,4-c]isoquinolin-1-yl)-N-methylindolizine-2-carboxamide;
2-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
3-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
N-(6-((2-aminoethyl)amino)-8,9-difluoro-1,4-dihydro-2H-pyrano[3,4-c]isoquinolin-1-yl)-5,6-difluoro-N-methyl-1H-indole-2-carboxamide;
N-(8,9-difluoro-6-oxo-1,4,5,6-tetrahydro-2H-pyrano[3,4-c]isoquinolin-1-yl)-6-fluoro-4-(1-hydroxyethyl)-N-methyl-1H-indole-2-carboxamide;
N-(8,9-difluoro-6-oxo-1,4,5,6-tetrahydro-2H-pyrano[3,4-c]isoquinolin-1-yl)-6-fluoro-4-(hydroxymethyl)-N-methyl-1H-indole-2-carboxamide;
7-bromo-N-(8,9-difluoro-6-oxo-1,4,5,6-tetrahydro-2H-pyrano[3,4-c]isoquinolin-1-yl)-N-methylfuro[3,2-c]pyridine-2-carboxamide;
3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-6-carboxamide;
3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-6-carboxamide;
3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-5-carboxamide;
3,4-dichloro-N-(8,9-difluoro-6-oxo-1,4,5,6-tetrahydro-2H-pyrano[3,4-c]isoquinolin-1-yl)-N-methylbenzamide;
N-(8,9-difluoro-6-oxo-1,4,5,6-tetrahydro-2H-pyrano[3,4-c]isoquinolin-1-yl)-6-fluoro-N-methyl-4-(methylsulfonamido)-1H-indole-2-carboxamide;
N-(8,9-difluoro-6-oxo-1,4,5,6-tetrahydro-2H-pyrano[3,4-c]isoquinolin-1-yl)-N-methyl-4-(trifluoromethoxy)-1H-indole-2-carboxamide;
N-(8,9-difluoro-6-oxo-1,4,5,6-tetrahydro-2H-pyrano[3,4-c]isoquinolin-1-yl)-6-methoxy-N-methyl-1H-indole-2-carboxamide;
5-bromo-N-(8,9-difluoro-6-oxo-1,4,5,6-tetrahydro-2H-pyrano[3,4-c]isoquinolin-1-yl)-N-methyl-6-(trifluoromethyl)nicotinamide;
3-chloro-N-(8,9-difluoro-6-oxo-1,4,5,6-tetrahydro-2H-pyrano[3,4-c]isoquinolin-1-yl)-N-methylindolizine-6-carboxamide;
N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-6-carboxamide;
N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-5-carboxamide;
N-(8,9-difluoro-6-oxo-1,4,5,6-tetrahydro-2H-pyrano[3,4-c]isoquinolin-1-yl)-3-methoxy-N-methyl-1H-indazole-5-carboxamide;
1-(5-chlorothiophen-3-yl)-N-(8,9-difluoro-6-oxo-1,4,5,6-tetrahydro-2H-pyrano[3,4-c]isoquinolin-1-yl)-N-methylazetidine-3-carboxamide;
7-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
4-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
3-chloro-N-(8,9-difluoro-6-oxo-1,4,5,6-tetrahydro-2H-pyrano[3,4-c]isoquinolin-1-yl)-N-methylindolizine-7-carboxamide;
N-(8,9-difluoro-6-oxo-1,4,5,6-tetrahydro-2H-pyrano[3,4-c]isoquinolin-1-yl)-7-(difluoromethyl)-N-methylindolizine-2-carboxamide;
N-(8,9-difluoro-6-oxo-1,4,5,6-tetrahydro-2H-pyrano[3,4-c]isoquinolin-1-yl)-6-(difluoromethyl)-N-methylindolizine-2-carboxamide;
di-tert-butyl ((2-((8,9-difluoro-6-oxo-1,4,5,6-tetrahydro-2H-pyrano[3,4-c]isoquinolin-1-yl)(methyl)carbamoyl)-5,6-difluoro-1H-indol-1-yl)methyl)phosphate;
5-chloro-N-(8,9-difluoro-6-oxo-1,4,5,6-tetrahydro-2H-pyrano[3,4-c]isoquinolin-1-yl)-N-methyl-6-(trifluoromethyl)nicotinamide;
5-chloro-N-(8-fluoro-6-oxo-1,4,5,6-tetrahydro-2H-pyrano[3,4-c]isoquinolin-1-yl)-N-methyl-6-(trifluoromethyl)nicotinamide;
N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
4-cyano-6-fluoro-N-(8-fluoro-6-oxo-1,4,5,6-tetrahydro-2H-pyrano[3,4-c]isoquinolin-1-yl)-N-methyl-1H-indole-2-carboxamide;
4-bromo-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
N-(8,9-difluoro-6-oxo-1,2,3,4,5,6-hexahydrobenzo[c][1,7]naphthyridin-1-yl)-7-(difluoromethyl)-N-methylindolizine-2-carboxamide;
N-(8,9-difluoro-6-oxo-1,2,3,4,5,6-hexahydrobenzo[c][1,7]naphthyridin-1-yl)-6-(difluoromethyl)-N-methylindolizine-2-carboxamide;
N-(8-fluoro-6-oxo-1,4,5,6-tetrahydro-2H-pyrano[3,4-c]isoquinolin-1-yl)-N-methylindolizine-6-carboxamide;
N-(8,9-difluoro-6-oxo-1,2,3,4,5,6-hexahydrobenzo[c][1,7]naphthyridin-1-yl)-N-methylindolizine-6-carboxamide;
4-ethyl-6-fluoro-N-(8-fluoro-6-oxo-1,4,5,6-tetrahydro-2H-pyrano[3,4-c]isoquinolin-1-yl)-N-methyl-1H-indole-2-carboxamide;
5-cyano-N-(8-fluoro-6-oxo-1,4,5,6-tetrahydro-2H-pyrano[3,4-c]isoquinolin-1-yl)-N-methyl-1H-indole-2-carboxamide;
4-cyano-N-(8,9-difluoro-6-oxo-1,2,3,4,5,6-hexahydrobenzo[c][1,7]naphthyridin-1-yl)-6-fluoro-N-methyl-1H-indole-2-carboxamide;
N-(8,9-difluoro-6-oxo-1,2,3,4,5,6-hexahydrobenzo[c][1,7]naphthyridin-1-yl)-4-ethyl-6-fluoro-N-methyl-1H-indole-2-carboxamide;
N-(8,9-difluoro-6-oxo-1,2,3,4,5,6-hexahydrobenzo[c][1,7]naphthyridin-1-yl)-N-methyl-5-(methylsulfonyl)-1H-indole-2-carboxamide;
5-cyano-N-(8,9-difluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-1-yl)-8-(difluoromethyl)-N-methylindolizine-2-carboxamide;
N-(8,9-difluoro-6-oxo-1,4,5,6-tetrahydro-2H-pyrano[3,4-c]isoquinolin-1-yl)-N-methyl-2,3-dihydro-1H-indene-5-carboxamide;
N-(8,9-difluoro-6-oxo-1,4,5,6-tetrahydro-2H-pyrano[3,4-c]isoquinolin-1-yl)-N-methylbenzo[d]oxazole-6-carboxamide;
4,6-dichloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
5,6-difluoro-N-methyl-N-(4,8,9-trifluoro-6-oxo-1,4,5,6-tetrahydro-2H-pyrano[3,4-c]isoquinolin-1-yl)-1H-indole-2-carboxamide;
N-(8,9-difluoro-6-oxo-1,4,5,6-tetrahydro-2H-pyrano[3,4-c]isoquinolin-1-yl)-5-fluoro-N-methyl-6-(trifluoromethyl)nicotinamide;
5-fluoro-N-(8-fluoro-6-oxo-1,4,5,6-tetrahydro-2H-pyrano[3,4-c]isoquinolin-1-yl)-N-methyl-6-(trifluoromethyl)nicotinamide;
3-chloro-N-(8,9-difluoro-6-oxo-1,4,5,6-tetrahydro-2H-pyrano[3,4-c]isoquinolin-1-yl)-N,1-dimethyl-1H-indazole-5-carboxamide;
3-chloro-N-(8,9-difluoro-6-oxo-1,4,5,6-tetrahydro-2H-pyrano[3,4-c]isoquinolin-1-yl)-1-ethyl-N-methyl-1H-indazole-5-carboxamide;
N-(8,9-difluoro-6-oxo-1,4,5,6-tetrahydro-2H-pyrano[3,4-c]isoquinolin-1-yl)-1-(difluoromethyl)-N,3-dimethyl-1H-indazole-5-carboxamide;
5-chloro-N-(8,9-difluoro-6-oxo-1,2,3,4,5,6-hexahydrobenzo[c][1,7]naphthyridin-1-yl)-N-methyl-6-(trifluoromethyl)nicotinamide;
N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
N-(8-fluoro-6-oxo-1,4,5,6-tetrahydro-2H-pyrano[3,4-c]isoquinolin-1-yl)-N,4-dimethyl-1H-indole-2-carboxamide;
4-chloro-N-(8-fluoro-6-oxo-1,4,5,6-tetrahydro-2H-pyrano[3,4-c]isoquinolin-1-yl)-N-methyl-1H-indole-2-carboxamide;
N-(8,9-difluoro-6-oxo-1,4,5,6-tetrahydro-2H-pyrano[3,4-c]isoquinolin-1-yl)-N-methylbenzo[d]oxazole-5-carboxamide;
N-(8,9-difluoro-6-oxo-1,4,5,6-tetrahydro-2H-pyrano[3,4-c]isoquinolin-1-yl)-N-methylbenzo[d]thiazole-5-carboxamide;
N-(8,9-difluoro-6-oxo-1,4,5,6-tetrahydro-2H-pyrano[3,4-c]isoquinolin-1-yl)-N-methylbenzo[d]thiazole-6-carboxamide;
N-(8,9-difluoro-6-oxo-1,4,5,6-tetrahydro-2H-pyrano[3,4-c]isoquinolin-1-yl)-N-methyl-1H-indazole-5-carboxamide;
N-(8,9-difluoro-6-oxo-1,2,3,4,5,6-hexahydrobenzo[c][1,7]naphthyridin-1-yl)-8-(difluoromethyl)-N-methylindolizine-2-carboxamide;
N-(8,9-difluoro-6-oxo-1,2,3,4,5,6-hexahydrobenzo[c][1,7]naphthyridin-1-yl)-5-(difluoromethyl)-N-methylindolizine-2-carboxamide;
N-(8,9-difluoro-6-oxo-1,2,3,4,5,6-hexahydrobenzo[c][1,7]naphthyridin-1-yl)-5-fluoro-N-methyl-6-(trifluoromethyl)nicotinamide;
N-(8,9-difluoro-6-oxo-1,4,5,6-tetrahydro-2H-pyrano[3,4-c]isoquinolin-1-yl)-3-(difluoromethyl)-N,1-dimethyl-1H-indazole-5-carboxamide;
N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
4-chloro-6-fluoro-N-(8-fluoro-6-oxo-1,4,5,6-tetrahydro-2H-pyrano[3,4-c]isoquinolin-1-yl)-N-methyl-1H-indole-2-carboxamide;
4-chloro-N-(8,9-difluoro-6-oxo-1,2,3,4,5,6-hexahydrobenzo[c][1,7]naphthyridin-1-yl)-N-methyl-1H-indole-2-carboxamide;
N-(8-fluoro-6-oxo-1,4,5,6-tetrahydro-2H-pyrano[3,4-c]isoquinolin-1-yl)-N-methyl-2,3-dihydro-1H-indene-5-carboxamide;
N-(8-fluoro-6-oxo-1,4,5,6-tetrahydro-2H-pyrano[3,4-c]isoquinolin-1-yl)-N-methylbenzo[d]thiazole-5-carboxamide;
N-(8-fluoro-6-oxo-1,4,5,6-tetrahydro-2H-pyrano[3,4-c]isoquinolin-1-yl)-N-methylbenzo[d]thiazole-6-carboxamide;
N-(8-fluoro-6-oxo-1,4,5,6-tetrahydro-2H-pyrano[3,4-c]isoquinolin-1-yl)-N-methyl-1H-indazole-5-carboxamide;
N-(8,9-difluoro-6-oxo-1,4,5,6-tetrahydro-2H-pyrano[3,4-c]isoquinolin-1-yl)-N-methyl-1H-indazole-6-carboxamide;
N-(8,9-difluoro-6-oxo-1,4,5,6-tetrahydro-2H-pyrano[3,4-c]isoquinolin-1-yl)-N-methyl-1H-benzo[d]imidazole-6-carboxamide;
2-chloro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
2-chloro-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
6-fluoro-N-(8-fluoro-6-oxo-1,4,5,6-tetrahydro-2H-pyrano[3,4-c]isoquinolin-1-yl)-N,4-dimethyl-1H-indole-2-carboxamide;
N-(8,9-difluoro-6-oxo-1,2,3,4,5,6-hexahydrobenzo[c][1,7]naphthyridin-1-yl)-N,4-dimethyl-1H-indole-2-carboxamide;
N-(8,9-difluoro-6-oxo-1,2,3,4,5,6-hexahydrobenzo[c][1,7]naphthyridin-1-yl)-6-fluoro-N,4-dimethyl-1H-indole-2-carboxamide;
4-chloro-N-(8,9-difluoro-6-oxo-1,2,3,4,5,6-hexahydrobenzo[c][1,7]naphthyridin-1-yl)-6-fluoro-N-methyl-1H-indole-2-carboxamide;
N-(8,9-difluoro-6-oxo-1,2,3,4,5,6-hexahydrobenzo[c][1,7]naphthyridin-1-yl)-N-methylbenzo[d]thiazole-5-carboxamide;
N-(8,9-difluoro-6-oxo-1,2,3,4,5,6-hexahydrobenzo[c][1,7]naphthyridin-1-yl)-N-methylbenzo[d]thiazole-6-carboxamide;
N-(8,9-difluoro-6-oxo-1,2,3,4,5,6-hexahydrophenanthridin-1-yl)-N-methyl-1H-indole-2-carboxamide;
2-cyano-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
N-(8-fluoro-6-oxo-1,4,5,6-tetrahydro-2H-pyrano[3,4-c]isoquinolin-1-yl)-N-methyl-1H-indazole-6-carboxamide;
N-(8,9-difluoro-6-oxo-1,2,3,4,5,6-hexahydrobenzo[c][1,7]naphthyridin-1-yl)-N-methyl-2,3-dihydro-1H-indene-5-carboxamide;
N-(8,9-difluoro-6-oxo-1,2,3,4,5,6-hexahydrobenzo[c][1,7]naphthyridin-1-yl)-N-methyl-1H-indazole-5-carboxamide;
N-(8-fluoro-6-oxo-1,2,3,4,5,6-hexahydrophenanthridin-1-yl)-N-methyl-1H-indole-2-carboxamide;
2-chloro-N-(8,9-difluoro-4,6-dioxo-1,4,5,6-tetrahydro-2H-pyrano[3,4-c]isoquinolin-1-yl)-N-methyl-4H-thieno[3,2-b]pyrrole-5-carboxamide;
N-(8,9-difluoro-4,6-dioxo-1,4,5,6-tetrahydro-2H-pyrano[3,4-c]isoquinolin-1-yl)-6-(difluoromethyl)-5-fluoro-N-methyl-1H-indole-2-carboxamide;
6-(difluoromethyl)-5-fluoro-N-(8-fluoro-6-oxo-1,2,3,4,5,6-hexahydrophenanthridin-1-yl)-N-methyl-1H-indole-2-carboxamide;
N-(8,9-difluoro-6-oxo-1,2,3,4,5,6-hexahydrophenanthridin-1-yl)-5-fluoro-N-methyl-1H-indole-2-carboxamide;
N-(8,9-difluoro-6-oxo-1,2,3,4,5,6-hexahydrophenanthridin-1-yl)-5,6-difluoro-N-methyl-1H-indole-2-carboxamide;
N-(8,9-difluoro-6-oxo-1,2,3,4,5,6-hexahydrophenanthridin-1-yl)-6-(difluoromethyl)-5-fluoro-N-methyl-1H-indole-2-carboxamide;
N-(8,9-difluoro-6-oxo-1,2,3,4,5,6-hexahydrophenanthridin-1-yl)-4-(difluoromethyl)-6-fluoro-N-methyl-1H-indole-2-carboxamide;
N-(8,9-difluoro-6-oxo-1,2,3,4,5,6-hexahydrophenanthridin-1-yl)-N,4-dimethyl-1H-indole-2-carboxamide;
N-(8,9-difluoro-6-oxo-1,2,3,4,5,6-hexahydrophenanthridin-1-yl)-6-fluoro-N,4-dimethyl-1H-indole-2-carboxamide;
1-((4-bromo-3-fluorobenzyl)(methyl)amino)-8,9-difluoro-1,5-dihydro-2H-pyrano[3,4-c]isoquinolin-6(4H)-one;
2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
5-fluoro-N-(8-fluoro-6-oxo-1,2,3,4,5,6-hexahydrophenanthridin-1-yl)-N-methyl-1H-indole-2-carboxamide;
5,6-difluoro-N-(8-fluoro-6-oxo-1,2,3,4,5,6-hexahydrophenanthridin-1-yl)-N-methyl-1H-indole-2-carboxamide;
N-(8-fluoro-6-oxo-1,2,3,4,5,6-hexahydrophenanthridin-1-yl)-N,4-dimethyl-1H-indole-2-carboxamide;
6-fluoro-N-(8-fluoro-6-oxo-1,2,3,4,5,6-hexahydrophenanthridin-1-yl)-N,4-dimethyl-1H-indole-2-carboxamide;
4-(difluoromethyl)-6-fluoro-N-(8-fluoro-6-oxo-1,2,3,4,5,6-hexahydrophenanthridin-1-yl)-N-methyl-1H-indole-2-carboxamide;
1-(((5,6-difluoro-1H-indol-2-yl)methyl)(methyl)amino)-8,9-difluoro-1,5-dihydro-2H-pyrano[3,4-c]isoquinolin-6(4H)-one;
N-(8,9-difluoro-6-oxo-1,2,3,4,5,6-hexahydrobenzo[c][1,7]naphthyridin-1-yl)-N-methyl-1H-indazole-6-carboxamide;
2-fluoro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
N-(8,9-difluoro-6-oxo-1,2,3,4,5,6-hexahydrobenzo[c][1,7]naphthyridin-1-yl)-2-fluoro-N-methyl-4H-thieno[3,2-b]pyrrole-5-carboxamide;
N-(8,9-difluoro-4-hydroxy-6-oxo-1,2,3,4,5,6-hexahydrophenanthridin-1-yl)-5-fluoro-N-methyl-1H-indole-2-carboxamide;
N-(8,9-difluoro-4-hydroxy-6-oxo-1,2,3,4,5,6-hexahydrophenanthridin-1-yl)-5,6-difluoro-N-methyl-1H-indole-2-carboxamide;
N-(8,9-difluoro-4-hydroxy-6-oxo-1,2,3,4,5,6-hexahydrophenanthridin-1-yl)-N-methyl-1H-indole-2-carboxamide;
N-(8,9-difluoro-6-oxo-1,2,3,4,5,6-hexahydrobenzo[c][1,7]naphthyridin-1-yl)-N-methylindolizine-2-carboxamide;
8-chloro-N-(8,9-difluoro-6-oxo-1,2,3,4,5,6-hexahydrobenzo[c][1,7]naphthyridin-1-yl)-N-methylindolizine-2-carboxamide;
N-(8,9-Difluoro-6-oxo-1,4,5,6-tetrahydro-2H-pyrano[3,4-c]isoquinolin-1-yl)-2,2-difluoro-N-methyl-2-phenylacetamide;
N-(8,9-Difluoro-6-oxo-1,4,5,6-tetrahydro-2H-pyrano[3,4-c]isoquinolin-1-yl-1-d)-5,6-difluoro-N-(methyl-13C-d3)-1H-indole-2-carboxamide;
N-(8,9-Difluoro-6-oxo-1,4,5,6-tetrahydro-2H-pyrano[3,4-c]isoquinolin-1-yl-1-d)-6-(difluoromethyl)-5-fluoro-N-(methyl-13C-d3)-1H-indole-2-carboxamide; and
2-Chloro-N-(8,9-difluoro-6-oxo-1,4,5,6-tetrahydro-2H-pyrano[3,4-c]isoquinolin-1-yl-1-d)-N-(methyl-13C-d3)-4H-thieno[3,2-b]pyrrole-5-carboxamide;
or a salt, solvate, prodrug, isotopically labelled derivative, stereoisomer, or tautomer thereof, or any mixtures thereof.

13. The compound of claim 1, which is at least one selected from the group consisting of:
(R)-N-(8-fluoro-6-oxo-1,4,5,6-tetrahydro-2H-pyrano[3,4-c]isoquinolin-1-yl)-N-methyl-1H-indole-2-carboxamide;
(S)-N-(8-fluoro-6-oxo-1,4,5,6-tetrahydro-2H-pyrano[3,4-c]isoquinolin-1-yl)-N-methyl-1H-indole-2-carboxamide;
(2S)-N-(8-fluoro-6-oxo-1,4,5,6-tetrahydro-2H-pyrano[3,4-c]isoquinolin-1-yl)-N-methylindoline-2-carboxamide;
(2S)-N-(8-fluoro-6-oxo-1,4,5,6-tetrahydro-2H-pyrano[3,4-c]isoquinolin-(1R)-yl)-N-methylindoline-2-carboxamide;
(2S)-N-(8-fluoro-6-oxo-1,4,5,6-tetrahydro-2H-pyrano[3,4-c]isoquinolin-(1 S)-yl)-N-methylindoline-2-carboxamide;
(2R)-N-(8-fluoro-6-oxo-1,4,5,6-tetrahydro-2H-pyrano[3,4-c]isoquinolin-1-yl)-N-methylindoline-2-carboxamide;
(2R)-N-(8-fluoro-6-oxo-1,4,5,6-tetrahydro-2H-pyrano[3,4-c]isoquinolin-(1R)-yl)-N-methylindoline-2-carboxamide;
(2R)-N-(8-fluoro-6-oxo-1,4,5,6-tetrahydro-2H-pyrano[3,4-c]isoquinolin-(1 S)-yl)-N-methylindoline-2-carboxamide;
(N-(8,9-difluoro-6-oxo-1,4,5,6-tetrahydro-2H-pyrano[3,4-c]isoquinolin-(1R)-yl)-5-fluoro-N-methylindoline-(2R)-carboxamide;
N-(8,9-difluoro-6-oxo-1,4,5,6-tetrahydro-2H-pyrano[3,4-c]isoquinolin-(1S)-yl)-5-fluoro-N-methylindoline-(2R)-carboxamide;
N-(8,9-difluoro-6-oxo-1,4,5,6-tetrahydro-2H-pyrano[3,4-c]isoquinolin-(1R)-yl)-5-fluoro-N-methylindoline-(2S)-carboxamide;
N-(8,9-difluoro-6-oxo-1,4,5,6-tetrahydro-2H-pyrano[3,4-c]isoquinolin-(1S)-yl)-5-fluoro-N-methylindoline-(2S)-carboxamide;
(R)-N-(8,9-difluoro-6-oxo-1,4,5,6-tetrahydro-2H-pyrano[3,4-c]isoquinolin-1-yl)-8-fluoro-N-methylindolizine-2-carboxamide;
(S)-N-(8,9-difluoro-6-oxo-1,4,5,6-tetrahydro-2H-pyrano[3,4-c]isoquinolin-1-yl)-8-fluoro-N-methylindolizine-2-carboxamide;
(R)-N-(8,9-difluoro-6-oxo-1,4,5,6-tetrahydro-2H-pyrano[3,4-c]isoquinolin-1-yl)-N-methylindolizine-2-carboxamide;
(S)-N-(8,9-difluoro-6-oxo-1,4,5,6-tetrahydro-2H-pyrano[3,4-c]isoquinolin-1-yl)-N-methylindolizine-2-carboxamide;
N-(8,9-difluoro-6-oxo-1,4,5,6-tetrahydro-2H-pyrano[3,4-c]isoquinolin-(1R)-yl)-4,6-difluoro-N-methylindoline-(2R)-carboxamide;
N-(8,9-difluoro-6-oxo-1,4,5,6-tetrahydro-2H-pyrano[3,4-c]isoquinolin-(1R)-yl)-4,6-difluoro-N-methylindoline-(2S)-carboxamide;
N-(8,9-difluoro-6-oxo-1,4,5,6-tetrahydro-2H-pyrano[3,4-c]isoquinolin-(1S)-yl)-4,6-difluoro-N-methylindoline-(2R)-carboxamide;
N-(8,9-difluoro-6-oxo-1,4,5,6-tetrahydro-2H-pyrano[3,4-c]isoquinolin-(1S)-yl)-4,6-difluoro-N-methylindoline-(2S)-carboxamide;
(S)-N-((S)-8,9-difluoro-6-oxo-1,4,5,6-tetrahydro-2H-pyrano[3,4-c]isoquinolin-1-yl)-4,6-difluoro-N-methylindoline-2-carboxamide;
(S)-N-((R)-8,9-difluoro-6-oxo-1,4,5,6-tetrahydro-2H-pyrano[3,4-c]isoquinolin-1-yl)-4,6-difluoro-N-methylindoline-2-carboxamide;
(R)-N-((S)-8,9-difluoro-6-oxo-1,4,5,6-tetrahydro-2H-pyrano[3,4-c]isoquinolin-1-yl)-4,6-difluoro-N-methylindoline-2-carboxamide;
(S)-N-((R)-8,9-difluoro-6-oxo-1,4,5,6-tetrahydro-2H-pyrano[3,4-c]isoquinolin-1-yl)-4,6-difluoro-N-methylindoline-2-carboxamide;
(R)-8-chloro-N-(8,9-difluoro-6-oxo-1,4,5,6-tetrahydro-2H-pyrano[3,4-c]isoquinolin-1-yl)-N-methylindolizine-2-carboxamide;
(S)-8-chloro-N-(8,9-difluoro-6-oxo-1,4,5,6-tetrahydro-2H-pyrano[3,4-c]isoquinolin-1-yl)-N-methylindolizine-2-carboxamide;
(R)-4-fluoro-N-(8-fluoro-6-oxo-1,4,5,6-tetrahydro-2H-pyrano[3,4-c]isoquinolin-1-yl)-N-methyl-1H-indole-2-carboxamide;
(S)-4-fluoro-N-(8-fluoro-6-oxo-1,4,5,6-tetrahydro-2H-pyrano[3,4-c]isoquinolin-1-yl)-N-methyl-1H-indole-2-carboxamide;
(R)-N-(8,9-difluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-1H-indole-2-carboxamide;
(R)-N-(8,9-difluoro-6-oxo-1,4,5,6-tetrahydro-2H-pyrano[3,4-c]isoquinolin-1-yl)-4-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-4-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-5-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-5-fluoro-N-methyl-1H-indole-2-carboxamide;
(R)-5-fluoro-N-(8-fluoro-6-oxo-1,4,5,6-tetrahydro-2H-pyrano[3,4-c]isoquinolin-1-yl)-N-methyl-1H-indole-2-carboxamide;
(S)-5-fluoro-N-(8-fluoro-6-oxo-1,4,5,6-tetrahydro-2H-pyrano[3,4-c]isoquinolin-1-yl)-N-methyl-1H-indole-2-carboxamide;
(R)-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-7-fluoro-N-(8-fluoro-6-oxo-1,4,5,6-tetrahydro-2H-pyrano[3,4-c]isoquinolin-1-yl)-N-methyl-1H-indole-2-carboxamide;
(S)-7-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4,6-difluoro-N-(8-fluoro-6-oxo-1,4,5,6-tetrahydro-2H-pyrano[3,4-c]isoquinolin-1-yl)-N-methyl-1H-indole-2-carboxamide;
(S)-4,6-difluoro-N-(8-fluoro-6-oxo-1,4,5,6-tetrahydro-2H-pyrano[3,4-c]isoquinolin-1-yl)-N-methyl-1H-indole-2-carboxamide;
(R)-4,5-difluoro-N-(8-fluoro-6-oxo-1,4,5,6-tetrahydro-2H-pyrano[3,4-c]isoquinolin-1-yl)-N-methyl-1H-indole-2-carboxamide;
(S)-4,5-difluoro-N-(8-fluoro-6-oxo-1,4,5,6-tetrahydro-2H-pyrano[3,4-c]isoquinolin-1-yl)-N-methyl-1H-indole-2-carboxamide;
(R)-N-(8,9-difluoro-6-oxo-1,4,5,6-tetrahydro-2H-pyrano[3,4-c]isoquinolin-1-yl)-7-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-7-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-5,6-difluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-4,6-difluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4,5-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-4,5-difluoro-N-methyl-1H-indole-2-carboxamide;
(R)-N-(8,9-difluoro-6-oxo-1,4,5,6-tetrahydro-2H-pyrano[3,4-c]isoquinolin-1-yl)-5,5-difluoro-N-methyl-4,5,6,7-tetrahydro-1H-indole-2-carboxamide;
(S)-N-(8,9-difluoro-6-oxo-1,4,5,6-tetrahydro-2H-pyrano[3,4-c]isoquinolin-1-yl)-5,5-difluoro-N-methyl-4,5,6,7-tetrahydro-1H-indole-2-carboxamide;
(R)-5,6-difluoro-N-(8-fluoro-6-oxo-1,4,5,6-tetrahydro-2H-pyrano[3,4-c]isoquinolin-1-yl)-N-methyl-1H-indole-2-carboxamide;
(S)-5,6-difluoro-N-(8-fluoro-6-oxo-1,4,5,6-tetrahydro-2H-pyrano[3,4-c]isoquinolin-1-yl)-N-methyl-1H-indole-2-carboxamide;
(R)-6-chloro-N-(8,9-difluoro-6-oxo-1,4,5,6-tetrahydro-2H-pyrano[3,4-c]isoquinolin-1-yl)-N-methylimidazo[1,2-a]pyridine-2-carboxamide;
(S)-6-chloro-N-(8,9-difluoro-6-oxo-1,4,5,6-tetrahydro-2H-pyrano[3,4-c]isoquinolin-1-yl)-N-methylimidazo[1,2-a]pyridine-2-carboxamide;
(R)-6-chloro-N-(8-fluoro-6-oxo-1,4,5,6-tetrahydro-2H-pyrano[3,4-c]isoquinolin-1-yl)-N-methylimidazo[1,2-a]pyridine-2-carboxamide;
(S)-6-chloro-N-(8-fluoro-6-oxo-1,4,5,6-tetrahydro-2H-pyrano[3,4-c]isoquinolin-1-yl)-N-methylimidazo[1,2-a]pyridine-2-carboxamide;
(R)-6-chloro-N-(8-fluoro-6-oxo-1,4,5,6-tetrahydro-2H-pyrano[3,4-c]isoquinolin-1-yl)-N-ethylimidazo[1,2-a]pyridine-2-carboxamide;
(S)-6-chloro-N-(8-fluoro-6-oxo-1,4,5,6-tetrahydro-2H-pyrano[3,4-c]isoquinolin-1-yl)-N-ethylimidazo[1,2-a]pyridine-2-carboxamide;
(R)-N-(8,9-difluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,2,3,4,5,6-hexahydrobenzo[c][1,7]naphthyridin-1-yl)-N-methyl-1H-indole-2-carboxamide;
N-(8,9-difluoro-6-oxo-1,4,5,6-tetrahydro-2H-pyrano[3,4-c]isoquinolin-(1R)-yl)-N,3,3-trimethylindoline-(2R)-carboxamide;
N-(8,9-difluoro-6-oxo-1,4,5,6-tetrahydro-2H-pyrano[3,4-c]isoquinolin-(1R)-yl)-N,3,3-trimethylindoline-(2S)-carboxamide;
N-(8,9-difluoro-6-oxo-1,4,5,6-tetrahydro-2H-pyrano[3,4-c]isoquinolin-(1S)-yl)-N,3,3-trimethylindoline-(2R)-carboxamide;
N-(8,9-difluoro-6-oxo-1,4,5,6-tetrahydro-2H-pyrano[3,4-c]isoquinolin-(1S)-yl)-N,3,3-trimethylindoline-(2S)-carboxamide;
(R)-N-(8-fluoro-3-methyl-6-oxo-1,2,3,4,5,6-hexahydrobenzo[c][1,7]naphthyridin-1-yl)-N-methyl-1H-indole-2-carboxamide;
(S)-N-(8-fluoro-3-methyl-6-oxo-1,2,3,4,5,6-hexahydrobenzo[c][1,7]naphthyridin-1-yl)-N-methyl-1H-indole-2-carboxamide;
(R)-N-(8,9-difluoro-3-methyl-6-oxo-1,2,3,4,5,6-hexahydrobenzo[c][1,7]naphthyridin-1-yl)-N-methyl-1H-indole-2-carboxamide;
(S)-N-(8,9-difluoro-3-methyl-6-oxo-1,2,3,4,5,6-hexahydrobenzo[c][1,7]naphthyridin-1-yl)-N-methyl-1H-indole-2-carboxamide;
(R)-N-(8,9-difluoro-6-oxo-1,4,5,6-tetrahydro-2H-pyrano[3,4-c]isoquinolin-1-yl)-N-methylpyrazolo[1,5-a]pyridine-2-carboxamide;
(S)-N-(8,9-difluoro-6-oxo-1,4,5,6-tetrahydro-2H-pyrano[3,4-c]isoquinolin-1-yl)-N-methylpyrazolo[1,5-a]pyridine-2-carboxamide;
(R)-N-(8,9-difluoro-6-oxo-1,4,5,6-tetrahydro-2H-pyrano[3,4-c]isoquinolin-1-yl)-N-methylpyrrolo[1,2-b]pyridazine-6-carboxamide;
(S)-N-(8,9-difluoro-6-oxo-1,4,5,6-tetrahydro-2H-pyrano[3,4-c]isoquinolin-1-yl)-N-methylpyrrolo[1,2-b]pyridazine-6-carboxamide;
(R)-N-(8,9-difluoro-6-oxo-1,4,5,6-tetrahydro-2H-pyrano[3,4-c]isoquinolin-1-yl)-N-methylquinoline-7-carboxamide;
(S)-N-(8,9-difluoro-6-oxo-1,4,5,6-tetrahydro-2H-pyrano[3,4-c]isoquinolin-1-yl)-N-methylquinoline-7-carboxamide;
(R)-N-(8,9-difluoro-6-oxo-1,4,5,6-tetrahydro-2H-pyrano[3,4-c]isoquinolin-1-yl)-N-methylquinoline-6-carboxamide;
(S)-N-(8,9-difluoro-6-oxo-1,4,5,6-tetrahydro-2H-pyrano[3,4-c]isoquinolin-1-yl)-N-methylquinoline-6-carboxamide;
(R)-N-(3-Acetyl-8-fluoro-6-oxo-1,2,3,4,5,6-hexahydrobenzo[c][1,7]naphthyridin-1-yl)-N-methyl-1H-indole-2-carboxamide;
(S)-N-(3-Acetyl-8-fluoro-6-oxo-1,2,3,4,5,6-hexahydrobenzo[c][1,7]naphthyridin-1-yl)-N-methyl-1H-indole-2-carboxamide;
(R)-N-(8,9-Difluoro-6-oxo-1,4,5,6-tetrahydro-2H-pyrano[3,4-c]isoquinolin-1-yl)-N-methyl-[1,2,4]triazolo[4,3-a]pyridine-6-carboxamide;
(S)-N-(8,9-Difluoro-6-oxo-1,4,5,6-tetrahydro-2H-pyrano[3,4-c]isoquinolin-1-yl)-N-methyl-[1,2,4]triazolo[4,3-a]pyridine-6-carboxamide;
(R)-N-(8,9-Difluoro-6-oxo-1,4,5,6-tetrahydro-2H-pyrano[3,4-c]isoquinolin-1-yl)-N-methylquinoline-3-carboxamide;
(S)-N-(8,9-Difluoro-6-oxo-1,4,5,6-tetrahydro-2H-pyrano[3,4-c]isoquinolin-1-yl)-N-methylquinoline-3-carboxamide;
(R)-N-(8,9-Difluoro-6-oxo-1,4,5,6-tetrahydro-2H-pyrano[3,4-c]isoquinolin-1-yl)-N-methylquinoxaline-6-carboxamide;
(S)-N-(8,9-Difluoro-6-oxo-1,4,5,6-tetrahydro-2H-pyrano[3,4-c]isoquinolin-1-yl)-N-methylquinoxaline-6-carboxamide;
(R)-N-(8,9-Difluoro-6-oxo-1,4,5,6-tetrahydro-2H-pyrano[3,4-c]isoquinolin-1-yl)-N-methyl-6-(trifluoromethyl)nicotinamide;
(S)-N-(8,9-Difluoro-6-oxo-1,4,5,6-tetrahydro-2H-pyrano[3,4-c]isoquinolin-1-yl)-N-methyl-6-(trifluoromethyl)nicotinamide;
(R)-N-(8,9-Difluoro-6-oxo-1,4,5,6-tetrahydro-2H-pyrano[3,4-c]isoquinolin-1-yl)-3-fluoro-N-methyl-4-(trifluoromethyl)benzamide;
(S)-N-(8,9-Difluoro-6-oxo-1,4,5,6-tetrahydro-2H-pyrano[3,4-c]isoquinolin-1-yl)-3-fluoro-N-methyl-4-(trifluoromethyl)benzamide;
(R)-4-Chloro-N-(8,9-difluoro-6-oxo-1,4,5,6-tetrahydro-2H-pyrano[3,4-c]isoquinolin-1-yl)-3-fluoro-N-methylbenzamide;
(S)-4-Chloro-N-(8,9-difluoro-6-oxo-1,4,5,6-tetrahydro-2H-pyrano[3,4-c]isoquinolin-1-yl)-3-fluoro-N-methylbenzamide;
(R)-N-(8,9-Difluoro-6-oxo-1,4,5,6-tetrahydro-2H-pyrano[3,4-c]isoquinolin-1-yl)-3,4,5-trifluoro-N-methylbenzamide;
(S)-N-(8,9-Difluoro-6-oxo-1,4,5,6-tetrahydro-2H-pyrano[3,4-c]isoquinolin-1-yl)-3,4,5-trifluoro-N-methylbenzamide;
(R)-N-(8-Fluoro-6-oxo-1,2,3,4,5,6-hexahydrobenzo[c][1,7]naphthyridin-1-yl)-N-methyl-1H-indole-2-carboxamide;
(S)-N-(8-Fluoro-6-oxo-1,2,3,4,5,6-hexahydrobenzo[c][1,7]naphthyridin-1-yl)-N-methyl-1H-indole-2-carboxamide;
(R)-N-(8-Fluoro-3-(2-hydroxyethyl)-6-oxo-1,2,3,4,5,6-hexahydrobenzo[c][1,7]naphthyridin-1-yl)-N-methyl-1H-indole-2-carboxamide;
(S)-N-(8-Fluoro-3-(2-hydroxyethyl)-6-oxo-1,2,3,4,5,6-hexahydrobenzo[c][1,7]naphthyridin-1-yl)-N-methyl-1H-indole-2-carboxamide;
(R)-N-(3-Acetyl-8,9-difluoro-6-oxo-1,2,3,4,5,6-hexahydrobenzo[c][1,7]naphthyridin-1-yl)-N-methyl-1H-indole-2-carboxamide;
(S)-N-(3-Acetyl-8,9-difluoro-6-oxo-1,2,3,4,5,6-hexahydrobenzo[c][1,7]naphthyridin-1-yl)-N-methyl-1H-indole-2-carboxamide;
(R)-N-(8,9-Difluoro-6-oxo-1,4,5,6-tetrahydro-2H-pyrano[3,4-c]isoquinolin-1-yl)-5-fluoro-N-methylnicotinamide;
(S)-N-(8,9-Difluoro-6-oxo-1,4,5,6-tetrahydro-2H-pyrano[3,4-c]isoquinolin-1-yl)-5-fluoro-N-methylnicotinamide;
(R)-5-Chloro-N-(8,9-difluoro-6-oxo-1,4,5,6-tetrahydro-2H-pyrano[3,4-c]isoquinolin-1-yl)-N-methylnicotinamide;
(S)-5-Chloro-N-(8,9-difluoro-6-oxo-1,4,5,6-tetrahydro-2H-pyrano[3,4-c]isoquinolin-1-yl)-N-methylnicotinamide;
(R)-N-(8,9-Difluoro-6-oxo-1,4,5,6-tetrahydro-2H-pyrano[3,4-c]isoquinolin-1-yl)-2-fluoro-N-methylisonicotinamide;
(S)-N-(8,9-Difluoro-6-oxo-1,4,5,6-tetrahydro-2H-pyrano[3,4-c]isoquinolin-1-yl)-2-fluoro-N-methylisonicotinamide;
(R)-N-(8,9-Difluoro-6-oxo-1,4,5,6-tetrahydro-2H-pyrano[3,4-c]isoquinolin-1-yl)-3-(difluoromethyl)-N-methylbenzamide;
(S)-N-(8,9-Difluoro-6-oxo-1,4,5,6-tetrahydro-2H-pyrano[3,4-c]isoquinolin-1-yl)-3-(difluoromethyl)-N-methylbenzamide;
N-((S)-8,9-Difluoro-6-oxo-1,4,5,6-tetrahydro-2H-pyrano[3,4-c]isoquinolin-1-yl)-(2R)-hydroxy-N-methyl-3-phenylpropanamide;
N-((S)-8,9-Difluoro-6-oxo-1,4,5,6-tetrahydro-2H-pyrano[3,4-c]isoquinolin-1-yl)-(2S)-hydroxy-N-methyl-3-phenylpropanamide;
(R)-N-(8,9-Difluoro-3-(2-hydroxyethyl)-6-oxo-1,2,3,4,5,6-hexahydrobenzo[c][1,7]naphthyridin-1-yl)-N-methyl-1H-indole-2-carboxamide;
(S)-N-(8,9-Difluoro-3-(2-hydroxyethyl)-6-oxo-1,2,3,4,5,6-hexahydrobenzo[c][1,7]naphthyridin-1-yl)-N-methyl-1H-indole-2-carboxamide;
(R)-N-(8,9-Difluoro-6-oxo-1,4,5,6-tetrahydro-2H-pyrano[3,4-c]isoquinolin-1-yl)-N-methylbenzamide;
(S)-N-(8,9-Difluoro-6-oxo-1,4,5,6-tetrahydro-2H-pyrano[3,4-c]isoquinolin-1-yl)-N-methylbenzamide;
(R)-N-(8,9-Difluoro-6-oxo-1,4,5,6-tetrahydro-2H-pyrano[3,4-c]isoquinolin-1-yl)-3,4-difluoro-N-methylbenzamide;
(S)-N-(8,9-Difluoro-6-oxo-1,4,5,6-tetrahydro-2H-pyrano[3,4-c]isoquinolin-1-yl)-3,4-difluoro-N-methylbenzamide;
N-((S)-8,9-Difluoro-6-oxo-1,4,5,6-tetrahydro-2H-pyrano[3,4-c]isoquinolin-1-yl)-(2R)-hydroxy-N-methyl-2-phenylpropanamide;
N-((S)-8,9-Difluoro-6-oxo-1,4,5,6-tetrahydro-2H-pyrano[3,4-c]isoquinolin-1-yl)-(2S)-hydroxy-N-methyl-2-phenylpropanamide;
N-((R)-8,9-Difluoro-6-oxo-1,4,5,6-tetrahydro-2H-pyrano[3,4-c]isoquinolin-1-yl)-(2R)-hydroxy-N-methyl-2-phenylpropanamide;
N-((R)-8,9-Difluoro-6-oxo-1,4,5,6-tetrahydro-2H-pyrano[3,4-c]isoquinolin-1-yl)-(2S)-hydroxy-N-methyl-2-phenylpropanamide; N-((S)-8,9-Difluoro-6-oxo-1,4,5,6-tetrahydro-2H-pyrano[3,4-c]isoquinolin-1-yl)-(2R)-hydroxy-N-methyl-2-phenylacetamide;
N-((S)-8,9-Difluoro-6-oxo-1,4,5,6-tetrahydro-2H-pyrano[3,4-c]isoquinolin-1-yl)-(2S)-hydroxy-N-methyl-2-phenylacetamide;
N-((R)-8,9-Difluoro-6-oxo-1,4,5,6-tetrahydro-2H-pyrano[3,4-c]isoquinolin-1-yl)-(2R)-hydroxy-N-methyl-2-phenylacetamide;
N-((R)-8,9-Difluoro-6-oxo-1,4,5,6-tetrahydro-2H-pyrano[3,4-c]isoquinolin-1-yl)-(2S)-hydroxy-N-methyl-2-phenylacetamide;
(R)-N-(8,9-Difluoro-6-oxo-1,4,5,6-tetrahydro-2H-pyrano[3,4-c]isoquinolin-1-yl)-4,5,6-trifluoro-N-methyl-1H-indole-2-carboxamide;
(S)-N-(8,9-Difluoro-6-oxo-1,4,5,6-tetrahydro-2H-pyrano[3,4-c]isoquinolin-1-yl)-4,5,6-trifluoro-N-methyl-1H-indole-2-carboxamide;
(R)-N-(8,9-Difluoro-6-oxo-1,4,5,6-tetrahydro-2H-pyrano[3,4-c]isoquinolin-1-yl)-N-methyl-4-(trifluoromethyl)benzamide;
(S)-N-(8,9-Difluoro-6-oxo-1,4,5,6-tetrahydro-2H-pyrano[3,4-c]isoquinolin-1-yl)-N-methyl-4-(trifluoromethyl)benzamide;
(R)-4-Chloro-N-(8,9-difluoro-6-oxo-1,4,5,6-tetrahydro-2H-pyrano[3,4-c]isoquinolin-1-yl)-N-methylbenzamide;
(S)-4-Chloro-N-(8,9-difluoro-6-oxo-1,4,5,6-tetrahydro-2H-pyrano[3,4-c]isoquinolin-1-yl)-N-methylbenzamide;
(R)-4-Cyano-N-(8,9-difluoro-6-oxo-1,4,5,6-tetrahydro-2H-pyrano[3,4-c]isoquinolin-1-yl)-3-fluoro-N-methylbenzamide;
(S)-4-Cyano-N-(8,9-difluoro-6-oxo-1,4,5,6-tetrahydro-2H-pyrano[3,4-c]isoquinolin-1-yl)-3-fluoro-N-methylbenzamide;
(R)-N-(8,9-Difluoro-6-oxo-1,2,3,4,5,6-hexahydrobenzo[c][1,7]naphthyridin-1-yl)-5-fluoro-N-methyl-1H-indole-2-carboxamide;
(S)-N-(8,9-Difluoro-6-oxo-1,2,3,4,5,6-hexahydrobenzo[c][1,7]naphthyridin-1-yl)-5-fluoro-N-methyl-1H-indole-2-carboxamide;
(R)-N-(8,9-Difluoro-6-oxo-1,4,5,6-tetrahydro-2H-thiopyrano[3,4-c]isoquinolin-1-yl)-8-fluoro-N-methylindolizine-2-carboxamide;
(S)-N-(8,9-Difluoro-6-oxo-1,4,5,6-tetrahydro-2H-thiopyrano[3,4-c]isoquinolin-1-yl)-8-fluoro-N-methylindolizine-2-carboxamide;
(R)-N-(8,9-Difluoro-6-oxo-1,2,3,4,5,6-hexahydrobenzo[c][1,7]naphthyridin-1-yl)-4-fluoro-N-methyl-1H-indole-2-carboxamide;
(S)-N-(8,9-Difluoro-6-oxo-1,2,3,4,5,6-hexahydrobenzo[c][1,7]naphthyridin-1-yl)-4-fluoro-N-methyl-1H-indole-2-carboxamide;
(R)-N-(8,9-Difluoro-6-oxo-1,2,3,4,5,6-hexahydrobenzo[c][1,7]naphthyridin-1-yl)-6-fluoro-N-methyl-1H-indole-2-carboxamide;
(S)-N-(8,9-Difluoro-6-oxo-1,2,3,4,5,6-hexahydrobenzo[c][1,7]naphthyridin-1-yl)-6-fluoro-N-methyl-1H-indole-2-carboxamide;
(R)-N-(8,9-Difluoro-6-oxo-1,4,5,6-tetrahydro-2H-pyrano[3,4-c]isoquinolin-1-yl)-3-(difluoromethyl)-4-fluoro-N-methylbenzamide;
(S)-N-(8,9-Difluoro-6-oxo-1,4,5,6-tetrahydro-2H-pyrano[3,4-c]isoquinolin-1-yl)-3-(difluoromethyl)-4-fluoro-N-methylbenzamide;
(R)-N-(8,9-Difluoro-6-oxo-1,4,5,6-tetrahydro-2H-pyrano[3,4-c]isoquinolin-1-yl)-4-(difluoromethyl)-3-fluoro-N-methylbenzamide;
(S)-N-(8,9-Difluoro-6-oxo-1,4,5,6-tetrahydro-2H-pyrano[3,4-c]isoquinolin-1-yl)-4-(difluoromethyl)-3-fluoro-N-methylbenzamide;
(R)-N-(8,9-Difluoro-6-oxo-1,4,5,6-tetrahydro-2H-pyrano[3,4-c]isoquinolin-1-yl)-4-(difluoromethyl)-N-methylbenzamide;
(S)-N-(8,9-Difluoro-6-oxo-1,4,5,6-tetrahydro-2H-pyrano[3,4-c]isoquinolin-1-yl)-4-(difluoromethyl)-N-methylbenzamide;
(R)-3-Chloro-N-(8,9-difluoro-6-oxo-1,4,5,6-tetrahydro-2H-pyrano[3,4-c]isoquinolin-1-yl)-4-fluoro-N-methylbenzamide;
(S)-3-Chloro-N-(8,9-difluoro-6-oxo-1,4,5,6-tetrahydro-2H-pyrano[3,4-c]isoquinolin-1-yl)-4-fluoro-N-methylbenzamide;
(R)-N-(8,9-Difluoro-6-oxo-1,2,3,4,5,6-hexahydrobenzo[c][1,7]naphthyridin-1-yl)-7-fluoro-N-methyl-1H-indole-2-carboxamide;
(S)-N-(8,9-Difluoro-6-oxo-1,2,3,4,5,6-hexahydrobenzo[c][1,7]naphthyridin-1-yl)-7-fluoro-N-methyl-1H-indole-2-carboxamide;
(R)-N-(8,9-Difluoro-6-oxo-1,2,3,4,5,6-hexahydrobenzo[c][1,7]naphthyridin-1-yl)-5,6-difluoro-N-methyl-1H-indole-2-carboxamide;
(S)-N-(8,9-Difluoro-6-oxo-1,2,3,4,5,6-hexahydrobenzo[c][1,7]naphthyridin-1-yl)-5,6-difluoro-N-methyl-1H-indole-2-carboxamide;
(R)-N-(8,9-Difluoro-6-oxo-1,2,3,4,5,6-hexahydrobenzo[c][1,7]naphthyridin-1-yl)-4,6-difluoro-N-methyl-1H-indole-2-carboxamide;
(S)-N-(8,9-Difluoro-6-oxo-1,2,3,4,5,6-hexahydrobenzo[c][1,7]naphthyridin-1-yl)-4,6-difluoro-N-methyl-1H-indole-2-carboxamide;
(R)-N-(8,9-Difluoro-6-oxo-1,2,3,4,5,6-hexahydrobenzo[c][1,7]naphthyridin-1-yl)-4,5-difluoro-N-methyl-1H-indole-2-carboxamide;
(S)-N-(8,9-Difluoro-6-oxo-1,2,3,4,5,6-hexahydrobenzo[c][1,7]naphthyridin-1-yl)-4,5-difluoro-N-methyl-1H-indole-2-carboxamide;
(R)-6-Chloro-N-(8,9-difluoro-6-oxo-1,2,3,4,5,6-hexahydrobenzo[c][1,7]naphthyridin-1-yl)-N-methylindolizine-2-carboxamide;
(S)-6-Chloro-N-(8,9-difluoro-6-oxo-1,2,3,4,5,6-hexahydrobenzo[c][1,7]naphthyridin-1-yl)-N-methylindolizine-2-carboxamide;
(R)-N-(8,9-Difluoro-6-oxo-1,4,5,6-tetrahydro-2H-pyrano[3,4-c]isoquinolin-1-yl)-N-methylbenzo[d]thiazole-2-carboxamide;
(S)-N-(8,9-Difluoro-6-oxo-1,4,5,6-tetrahydro-2H-pyrano[3,4-c]isoquinolin-1-yl)-N-methylbenzo[d]thiazole-2-carboxamide;
(R)-N-(8,9-Difluoro-6-oxo-1,4,5,6-tetrahydro-2H-pyrano[3,4-c]isoquinolin-1-yl)-N-methylbenzo[d]oxazole-2-carboxamide;
(S)-N-(8,9-Difluoro-6-oxo-1,4,5,6-tetrahydro-2H-pyrano[3,4-c]isoquinolin-1-yl)-N-methylbenzo[d]oxazole-2-carboxamide;
(R)-N-(8,9-Difluoro-6-oxo-1,4,5,6-tetrahydro-2H-pyrano[3,4-c]isoquinolin-1-yl)-N-methyl-3-(trifluoromethyl)-1H-pyrazole-5-carboxamide;
(S)-N-(8,9-Difluoro-6-oxo-1,4,5,6-tetrahydro-2H-pyrano[3,4-c]isoquinolin-1-yl)-N-methyl-3-(trifluoromethyl)-1H-pyrazole-5-carboxamide;
(R)-N-(8,9-Difluoro-6-oxo-1,4,5,6-tetrahydro-2H-pyrano[3,4-c]isoquinolin-1-yl)-3-(4-fluorophenyl)-N-methyl-1H-pyrazole-5-carboxamide;
(S)-N-(8,9-Difluoro-6-oxo-1,4,5,6-tetrahydro-2H-pyrano[3,4-c]isoquinolin-1-yl)-3-(4-fluorophenyl)-N-methyl-1H-pyrazole-5-carboxamide;
(R)-N-(8,9-Difluoro-6-oxo-1,4,5,6-tetrahydro-2H-pyrano[3,4-c]isoquinolin-1-yl)-3-fluoro-N-methylbenzamide;
(S)-N-(8,9-Difluoro-6-oxo-1,4,5,6-tetrahydro-2H-pyrano[3,4-c]isoquinolin-1-yl)-3-fluoro-N-methylbenzamide;
(R)-3-Chloro-N-(8,9-difluoro-6-oxo-1,4,5,6-tetrahydro-2H-pyrano[3,4-c]isoquinolin-1-yl)-N-methylbenzamide;
(S)-3-Chloro-N-(8,9-difluoro-6-oxo-1,4,5,6-tetrahydro-2H-pyrano[3,4-c]isoquinolin-1-yl)-N-methylbenzamide;
(R)-3-Bromo-N-(8,9-difluoro-6-oxo-1,4,5,6-tetrahydro-2H-pyrano[3,4-c]isoquinolin-1-yl)-N-methylbenzamide;
(S)-3-Bromo-N-(8,9-difluoro-6-oxo-1,4,5,6-tetrahydro-2H-pyrano[3,4-c]isoquinolin-1-yl)-N-methylbenzamide;
(R)-3-Bromo-N-(8,9-difluoro-6-oxo-1,4,5,6-tetrahydro-2H-pyrano[3,4-c]isoquinolin-1-yl)-4-fluoro-N-methylbenzamide;
(S)-3-Bromo-N-(8,9-difluoro-6-oxo-1,4,5,6-tetrahydro-2H-pyrano[3,4-c]isoquinolin-1-yl)-4-fluoro-N-methylbenzamide;
(R)-4-Bromo-N-(8,9-difluoro-6-oxo-1,4,5,6-tetrahydro-2H-pyrano[3,4-c]isoquinolin-1-yl)-N-methylbenzamide;
(S)-4-Bromo-N-(8,9-difluoro-6-oxo-1,4,5,6-tetrahydro-2H-pyrano[3,4-c]isoquinolin-1-yl)-N-methylbenzamide;
(R)-3-Fluoro-N-methyl-N-(6-oxo-1,4,5,6-tetrahydro-2H-pyrano[3,4-c]isoquinolin-1-yl)-4-(trifluoromethyl)benzamide;
(S)-3-Fluoro-N-methyl-N-(6-oxo-1,4,5,6-tetrahydro-2H-pyrano[3,4-c]isoquinolin-1-yl)-4-(trifluoromethyl)benzamide;
(R)-3-Fluoro-N-methyl-N-(4-oxo-4,5,8,9-tetrahydro-6H-pyrano[3,4-b]thieno[3,4-d]pyridin-9-yl)-4-(trifluoromethyl)benzamide;
(S)-3-Fluoro-N-methyl-N-(4-oxo-4,5,8,9-tetrahydro-6H-pyrano[3,4-b]thieno[3,4-d]pyridin-9-yl)-4-(trifluoromethyl)benzamide;
(R)-N-(8-Cyano-6-oxo-1,4,5,6-tetrahydro-2H-pyrano[3,4-c]isoquinolin-1-yl)-3-fluoro-N-methyl-4-(trifluoromethyl)benzamide;
(S)-N-(8-Cyano-6-oxo-1,4,5,6-tetrahydro-2H-pyrano[3,4-c]isoquinolin-1-yl)-3-fluoro-N-methyl-4-(trifluoromethyl)benzamide;
(R)-3-Cyano-N-(8,9-difluoro-6-oxo-1,4,5,6-tetrahydro-2H-pyrano[3,4-c]isoquinolin-1-yl)-N-methylbenzamide;
(S)-3-Cyano-N-(8,9-difluoro-6-oxo-1,4,5,6-tetrahydro-2H-pyrano[3,4-c]isoquinolin-1-yl)-N-methylbenzamide;
(R)-N-(8,9-Difluoro-6-oxo-1,4,5,6-tetrahydro-2H-pyrano[3,4-c]isoquinolin-1-yl)-3-fluoro-N-methyl-4-(trifluoromethoxy)benzamide;
(S)-N-(8,9-Difluoro-6-oxo-1,4,5,6-tetrahydro-2H-pyrano[3,4-c]isoquinolin-1-yl)-3-fluoro-N-methyl-4-(trifluoromethoxy)benzamide;
(R)-N-(8,9-Difluoro-6-oxo-1,4,5,6-tetrahydro-2H-pyrano[3,4-c]isoquinolin-1-yl)-3-fluoro-N,4-dimethylbenzamide;
(S)-N-(8,9-Difluoro-6-oxo-1,4,5,6-tetrahydro-2H-pyrano[3,4-c]isoquinolin-1-yl)-3-fluoro-N,4-dimethylbenzamide;
2-(3-Chlorophenyl)-N-((S)-8,9-difluoro-6-oxo-1,4,5,6-tetrahydro-2H-pyrano[3,4-c]isoquinolin-1-yl)-(2R)-hydroxy-N-methylacetamide;
2-(3-Chlorophenyl)-N-((S)-8,9-difluoro-6-oxo-1,4,5,6-tetrahydro-2H-pyrano[3,4-c]isoquinolin-1-yl)-(2S)-hydroxy-N-methylacetamide;
(R)-N-(8,9-Difluoro-6-oxo-1,4,5,6-tetrahydro-2H-pyrano[3,4-c]isoquinolin-1-yl)-3,5-difluoro-N-methyl-4-(trifluoromethyl)benzamide;
(S)-N-(8,9-Difluoro-6-oxo-1,4,5,6-tetrahydro-2H-pyrano[3,4-c]isoquinolin-1-yl)-3,5-difluoro-N-methyl-4-(trifluoromethyl)benzamide;
(R)-5-Chloro-N-(8,9-difluoro-6-oxo-1,4,5,6-tetrahydro-2H-pyrano[3,4-c]isoquinolin-1-yl)-N-methylthiophene-3-carboxamide;
(S)-5-Chloro-N-(8,9-difluoro-6-oxo-1,4,5,6-tetrahydro-2H-pyrano[3,4-c]isoquinolin-1-yl)-N-methylthiophene-3-carboxamide;
(2R)-Amino-N-((R)-8,9-difluoro-6-oxo-1,4,5,6-tetrahydro-2H-pyrano[3,4-c]isoquinolin-1-yl)-(3R)-hydroxy-N-methylbutanamide;
(2R)-Amino-N-((R)-8,9-difluoro-6-oxo-1,4,5,6-tetrahydro-2H-pyrano[3,4-c]isoquinolin-1-yl)-(3S)-hydroxy-N-methylbutanamide;
(2S)-Amino-N-((R)-8,9-difluoro-6-oxo-1,4,5,6-tetrahydro-2H-pyrano[3,4-c]isoquinolin-1-yl)-(3R)-hydroxy-N-methylbutanamide;
(2S)-Amino-N-((R)-8,9-difluoro-6-oxo-1,4,5,6-tetrahydro-2H-pyrano[3,4-c]isoquinolin-1-yl)-(3S)-hydroxy-N-methylbutanamide;
(2R)-Amino-N-((S)-8,9-difluoro-6-oxo-1,4,5,6-tetrahydro-2H-pyrano[3,4-c]isoquinolin-1-yl)-(3R)-hydroxy-N-methylbutanamide;
(2R)-Amino-N-((S)-8,9-difluoro-6-oxo-1,4,5,6-tetrahydro-2H-pyrano[3,4-c]isoquinolin-1-yl)-(3S)-hydroxy-N-methylbutanamide;
(2S)-Amino-N-((S)-8,9-difluoro-6-oxo-1,4,5,6-tetrahydro-2H-pyrano[3,4-c]isoquinolin-1-yl)-(3R)-hydroxy-N-methylbutanamide;
(2S)-Amino-N-((S)-8,9-difluoro-6-oxo-1,4,5,6-tetrahydro-2H-pyrano[3,4-c]isoquinolin-1-yl)-(3S)-hydroxy-N-methylbutanamide;
(R)-3-Chloro-N-(8,9-difluoro-6-oxo-1,4,5,6-tetrahydro-2H-pyrano[3,4-c]isoquinolin-1-yl)-4-fluoro-N-methylbenzenesulfonamide;
(S)-3-Chloro-N-(8,9-difluoro-6-oxo-1,4,5,6-tetrahydro-2H-pyrano[3,4-c]isoquinolin-1-yl)-4-fluoro-N-methylbenzenesulfonamide;
(R)-3-Chloro-N-(8,9-difluoro-6-oxo-1,4,5,6-tetrahydro-2H-pyrano[3,4-c]isoquinolin-1-yl)-N-methylbenzenesulfonamide;
(S)-3-Chloro-N-(8,9-difluoro-6-oxo-1,4,5,6-tetrahydro-2H-pyrano[3,4-c]isoquinolin-1-yl)-N-methylbenzenesulfonamide;
(R)-3-Cyano-N-(8,9-difluoro-6-oxo-1,4,5,6-tetrahydro-2H-pyrano[3,4-c]isoquinolin-1-yl)-N-methylbenzenesulfonamide;
(S)-3-Cyano-N-(8,9-difluoro-6-oxo-1,4,5,6-tetrahydro-2H-pyrano[3,4-c]isoquinolin-1-yl)-N-methylbenzenesulfonamide;
(R)-3-Cyano-N-(8,9-difluoro-6-oxo-1,4,5,6-tetrahydro-2H-pyrano[3,4-c]isoquinolin-1-yl)-4-fluoro-N-methylbenzenesulfonamide;
(S)-3-Cyano-N-(8,9-difluoro-6-oxo-1,4,5,6-tetrahydro-2H-pyrano[3,4-c]isoquinolin-1-yl)-4-fluoro-N-methylbenzenesulfonamide;
(R)-N-(8,9-Difluoro-6-oxo-1,4,5,6-tetrahydro-2H-pyrano[3,4-c]isoquinolin-1-yl)-4-ethyl-3-fluoro-N-methylbenzamide;
(S)-N-(8,9-Difluoro-6-oxo-1,4,5,6-tetrahydro-2H-pyrano[3,4-c]isoquinolin-1-yl)-4-ethyl-3-fluoro-N-methylbenzamide;
(S)-N-(8,9-difluoro-6-oxo-1,4,5,6-tetrahydro-2H-pyrano[3,4-c]isoquinolin-1-yl)indolizine-2-carboxamide;
(R)-N-(8,9-difluoro-6-oxo-1,4,5,6-tetrahydro-2H-pyrano[3,4-c]isoquinolin-1-yl)indolizine-2-carboxamide;
(R,R)-N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
(R,S)-N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
(S,R)-N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
(S,S)-N-(8,9-difluoro-3-oxido-6-oxo-1,4,5,6-tetrahydro-2H-thiopyrano[3,4-c]isoquinolin-1-yl)-8-fluoro-N-methylindolizine-2-carboxamide;
(S)-N-(8,9-difluoro-3,3-dioxido-6-oxo-1,4,5,6-tetrahydro-2H-thiopyrano[3,4-c]isoquinolin-1-yl)-8-fluoro-N-methylindolizine-2-carboxamide;
(R)-N-(8,9-difluoro-3,3-dioxido-6-oxo-1,4,5,6-tetrahydro-2H-thiopyrano[3,4-c]isoquinolin-1-yl)-8-fluoro-N-methylindolizine-2-carboxamide;
(S)-N-(8,9-difluoro-6-oxo-1,4,5,6-tetrahydro-2H-pyrano[3,4-c]isoquinolin-1-yl)-4-(difluoromethoxy)-3-fluoro-N-methylbenzamide;
(R)-N-(8,9-difluoro-6-oxo-1,4,5,6-tetrahydro-2H-pyrano[3,4-c]isoquinolin-1-yl)-4-(difluoromethoxy)-3-fluoro-N-methylbenzamide;
(S)-N-(8,9-difluoro-6-oxo-1,4,5,6-tetrahydro-2H-pyrano[3,4-c]isoquinolin-1-yl)-4-(2-hydroxypropan-2-yl)-N-methylbenzamide;
(R)-N-(8,9-difluoro-6-oxo-1,4,5,6-tetrahydro-2H-pyrano[3,4-c]isoquinolin-1-yl)-4-(2-hydroxypropan-2-yl)-N-methylbenzamide;
(S)-N-(8,9-difluoro-6-oxo-1,4,5,6-tetrahydro-2H-pyrano[3,4-c]isoquinolin-1-yl)-3-(2-hydroxypropan-2-yl)-N-methylbenzamide;
(R)-N-(8,9-difluoro-6-oxo-1,4,5,6-tetrahydro-2H-pyrano[3,4-c]isoquinolin-1-yl)-3-(2-hydroxypropan-2-yl)-N-methylbenzamide;
(S)-N-(8,9-difluoro-6-oxo-1,4,5,6-tetrahydro-2H-pyrano[3,4-c]isoquinolin-1-yl)-5,6-difluoro-1H-indole-2-carboxamide;
(R)-N-(8,9-difluoro-6-oxo-1,4,5,6-tetrahydro-2H-pyrano[3,4-c]isoquinolin-1-yl)-5,6-difluoro-1H-indole-2-carboxamide;
(S)-N-(8,9-difluoro-6-oxo-1,4,5,6-tetrahydro-2H-pyrano[3,4-c]isoquinolin-1-yl)-4,6-difluoro-1H-indole-2-carboxamide;
(R)-N-(8,9-difluoro-6-oxo-1,4,5,6-tetrahydro-2H-pyrano[3,4-c]isoquinolin-1-yl)-4,6-difluoro-1H-indole-2-carboxamide;
(S)-N-(8,9-difluoro-6-oxo-1,4,5,6-tetrahydro-2H-pyrano[3,4-c]isoquinolin-1-yl)-3-fluoro-4-(trifluoromethyl)benzamide;
(R)-N-(8,9-difluoro-6-oxo-1,4,5,6-tetrahydro-2H-pyrano[3,4-c]isoquinolin-1-yl)-3-fluoro-4-(trifluoromethyl)benzamide;
(S)-4-chloro-N-(8,9-difluoro-6-oxo-1,4,5,6-tetrahydro-2H-pyrano[3,4-c]isoquinolin-1-yl)-3-fluorobenzamide;
(R)-4-chloro-N-(8,9-difluoro-6-oxo-1,4,5,6-tetrahydro-2H-pyrano[3,4-c]isoquinolin-1-yl)-3-fluorobenzamide;
(S)-N-(8,9-difluoro-6-oxo-1,2,3,4,5,6-hexahydrobenzo[c][1,7]naphthyridin-1-yl)-8-fluoro-N-methylindolizine-2-carboxamide;
(R)-N-(8,9-difluoro-6-oxo-1,2,3,4,5,6-hexahydrobenzo[c][1,7]naphthyridin-1-yl)-8-fluoro-N-methylindolizine-2-carboxamide;
(S)-N-(8,9-difluoro-6-oxo-1,2,3,4,5,6-hexahydrobenzo[c][1,7]naphthyridin-1-yl)-7-fluoro-N-methylindolizine-2-carboxamide;
(R)-N-(8,9-difluoro-6-oxo-1,2,3,4,5,6-hexahydrobenzo[c][1,7]naphthyridin-1-yl)-7-fluoro-N-methylindolizine-2-carboxamide;
(S)-4-bromo-N-(8,9-difluoro-6-oxo-1,2,3,4,5,6-hexahydrobenzo[c][1,7]naphthyridin-1-yl)-3,5-difluoro-N-methylbenzamide;
(R)-4-bromo-N-(8,9-difluoro-6-oxo-1,2,3,4,5,6-hexahydrobenzo[c][1,7]naphthyridin-1-yl)-3,5-difluoro-N-methylbenzamide;
(S)-4-bromo-3,5-difluoro-N-(8-fluoro-6-oxo-1,4,5,6-tetrahydro-2H-pyrano[3,4-c]isoquinolin-1-yl)-N-methylbenzamide;
(R)-4-bromo-3,5-difluoro-N-(8-fluoro-6-oxo-1,4,5,6-tetrahydro-2H-pyrano[3,4-c]isoquinolin-1-yl)-N-methylbenzamide;
(S)-4-chloro-N-(8,9-difluoro-6-oxo-1,2,3,4,5,6-hexahydrobenzo[c][1,7]naphthyridin-1-yl)-3,5-difluoro-N-methylbenzamide;
(R)-4-chloro-N-(8,9-difluoro-6-oxo-1,2,3,4,5,6-hexahydrobenzo[c][1,7]naphthyridin-1-yl)-3,5-difluoro-N-methylbenzamide;
(S)-4-bromo-3,5-difluoro-N-(8-fluoro-6-oxo-1,2,3,4,5,6-hexahydrobenzo[c][1,7]naphthyridin-1-yl)-N-methylbenzamide;
(R)-4-bromo-3,5-difluoro-N-(8-fluoro-6-oxo-1,2,3,4,5,6-hexahydrobenzo[c][1,7]naphthyridin-1-yl)-N-methylbenzamide;
(S)-4-bromo-N-(8,9-difluoro-6-oxo-1,4,5,6-tetrahydro-2H-pyrano[3,4-c]isoquinolin-1-yl)-3,5-difluoro-N-methylbenzamide;
(R)-4-bromo-N-(8,9-difluoro-6-oxo-1,4,5,6-tetrahydro-2H-pyrano[3,4-c]isoquinolin-1-yl)-3,5-difluoro-N-methylbenzamide;
(S)-4-chloro-N-(8,9-difluoro-6-oxo-1,4,5,6-tetrahydro-2H-pyrano[3,4-c]isoquinolin-1-yl)-3,5-difluoro-N-methylbenzamide;
(R)-4-chloro-N-(8,9-difluoro-6-oxo-1,4,5,6-tetrahydro-2H-pyrano[3,4-c]isoquinolin-1-yl)-3,5-difluoro-N-methylbenzamide;
(S)-4-chloro-3,5-difluoro-N-(8-fluoro-6-oxo-1,4,5,6-tetrahydro-2H-pyrano[3,4-c]isoquinolin-1-yl)-N-methylbenzamide;
(R)-4-chloro-3,5-difluoro-N-(8-fluoro-6-oxo-1,4,5,6-tetrahydro-2H-pyrano[3,4-c]isoquinolin-1-yl)-N-methylbenzamide;
(S)-4-chloro-3,5-difluoro-N-(8-fluoro-6-oxo-1,2,3,4,5,6-hexahydrobenzo[c][1,7]naphthyridin-1-yl)-N-methylbenzamide;
(R)-4-chloro-3,5-difluoro-N-(8-fluoro-6-oxo-1,2,3,4,5,6-hexahydrobenzo[c][1,7]naphthyridin-1-yl)-N-methylbenzamide;
(S)-4-(difluoromethyl)-3,5-difluoro-N-(8-fluoro-6-oxo-1,4,5,6-tetrahydro-2H-pyrano[3,4-c]isoquinolin-1-yl)-N-methylbenzamide;
(R)-4-(difluoromethyl)-3,5-difluoro-N-(8-fluoro-6-oxo-1,4,5,6-tetrahydro-2H-pyrano[3,4-c]isoquinolin-1-yl)-N-methylbenzamide;
(S)-N-(8,9-difluoro-6-oxo-1,4,5,6-tetrahydro-2H-pyrano[3,4-c]isoquinolin-1-yl)-4-(difluoromethyl)-3,5-difluoro-N-methylbenzamide;
(R)-N-(8,9-difluoro-6-oxo-1,4,5,6-tetrahydro-2H-pyrano[3,4-c]isoquinolin-1-yl)-4-(difluoromethyl)-3,5-difluoro-N-methylbenzamide;
(S)-4-(difluoromethyl)-N-(8-fluoro-6-oxo-1,4,5,6-tetrahydro-2H-pyrano[3,4-c]isoquinolin-1-yl)-N-methyl-1H-indole-2-carboxamide;
(R)-4-(difluoromethyl)-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,2,3,4,5,6-hexahydrobenzo[c][1,7]naphthyridin-1-yl)-4-(difluoromethyl)-3,5-difluoro-N-methylbenzamide;
(R)-N-(8,9-difluoro-6-oxo-1,2,3,4,5,6-hexahydrobenzo[c][1,7]naphthyridin-1-yl)-4-(difluoromethyl)-3,5-difluoro-N-methylbenzamide;
(S)-4-(difluoromethyl)-3,5-difluoro-N-(8-fluoro-6-oxo-1,2,3,4,5,6-hexahydrobenzo[c][1,7]naphthyridin-1-yl)-N-methylbenzamide;
(R)-4-(difluoromethyl)-3,5-difluoro-N-(8-fluoro-6-oxo-1,2,3,4,5,6-hexahydrobenzo[c][1,7]naphthyridin-1-yl)-N-methylbenzamide;
N-((1S)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1S,4R)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1S,4S)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1R)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1R,4R)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
N-((1R,4S)-8,9-difluoro-4-hydroxy-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-N,1-dimethyl-1H-pyrazole-4-carboxamide;
(R)-N-(8,9-difluoro-6-oxo-1,4,5,6-tetrahydro-2H-pyrano[3,4-c]isoquinolin-1-yl)-N,1-dimethyl-1H-pyrazole-4-carboxamide;
(S)-N-(8,9-difluoro-6-oxo-1,4,5,6-tetrahydro-2H-pyrano[3,4-c]isoquinolin-1-yl)-N-methyl-2-phenylacrylamide;
(R)-N-(8,9-difluoro-6-oxo-1,4,5,6-tetrahydro-2H-pyrano[3,4-c]isoquinolin-1-yl)-N-methyl-2-phenylacrylamide;
(S)-N-(8,9-difluoro-6-oxo-1,4,5,6-tetrahydro-2H-pyrano[3,4-c]isoquinolin-1-yl)-5-fluoro-N-methyl-1H-pyrrolo[2,3-b]pyridine-2-carboxamide;
(R)-N-(8,9-difluoro-6-oxo-1,4,5,6-tetrahydro-2H-pyrano[3,4-c]isoquinolin-1-yl)-5-fluoro-N-methyl-1H-pyrrolo[2,3-b]pyridine-2-carboxamide;
(S)-5-chloro-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
(R)-5-chloro-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
(S)-5-bromo-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
(R)-5-bromo-N-(8,9-difluoro-6-oxo-1,4,5,6-tetrahydro-2H-pyrano[3,4-c]isoquinolin-1-yl)-N-methyl-1H-pyrrolo[2,3-b]pyridine-2-carboxamide;
(S)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-3-carboxamide;
(R)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-3-carboxamide;
(S)-5-bromo-N-(8,9-difluoro-6-oxo-1,4,5,6-tetrahydro-2H-pyrano[3,4-c]isoquinolin-1-yl)-N-methylthiophene-3-carboxamide;
(R)-5-bromo-N-(8,9-difluoro-6-oxo-1,4,5,6-tetrahydro-2H-pyrano[3,4-c]isoquinolin-1-yl)-N-methylthiophene-3-carboxamide;
(S)-N-(8,9-difluoro-6-oxo-1,4,5,6-tetrahydro-2H-pyrano[3,4-c]isoquinolin-1-yl)-N-methyl-2,3-dihydro-1H-indene-2-carboxamide;
(R)-N-(8,9-difluoro-6-oxo-1,4,5,6-tetrahydro-2H-pyrano[3,4-c]isoquinolin-1-yl)-N-methyl-2,3-dihydro-1H-indene-2-carboxamide;
(S)-1-(tert-butyl)-N-(8,9-difluoro-6-oxo-1,4,5,6-tetrahydro-2H-pyrano[3,4-c]isoquinolin-1-yl)-N-methyl-1H-pyrazole-4-carboxamide;
(R)-1-(tert-butyl)-N-(8,9-difluoro-6-oxo-1,4,5,6-tetrahydro-2H-pyrano[3,4-c]isoquinolin-1-yl)-N-methyl-1H-pyrazole-4-carboxamide;
(S)-N-(8,9-difluoro-6-oxo-1,4,5,6-tetrahydro-2H-pyrano[3,4-c]isoquinolin-1-yl)-N-methyl-1-(trifluoromethyl)-1H-pyrazole-4-carboxamide;
(R)-N-(8,9-difluoro-6-oxo-1,4,5,6-tetrahydro-2H-pyrano[3,4-c]isoquinolin-1-yl)-N-methyl-1-(trifluoromethyl)-1H-pyrazole-4-carboxamide;
(S)-4-bromo-N-(8,9-difluoro-6-oxo-1,4,5,6-tetrahydro-2H-pyrano[3,4-c]isoquinolin-1-yl)-N-methyl-1H-pyrrole-2-carboxamide;
(R)-4-bromo-N-(8,9-difluoro-6-oxo-1,4,5,6-tetrahydro-2H-pyrano[3,4-c]isoquinolin-1-yl)-N-methyl-1H-pyrrole-2-carboxamide;
(S)-2-amino-2-(4-chlorophenyl)-N-((S)-8,9-difluoro-6-oxo-1,4,5,6-tetrahydro-2H-pyrano[3,4-c]isoquinolin-1-yl)-N-methylacetamide;
(R)-2-amino-2-(4-chlorophenyl)-N-((S)-8,9-difluoro-6-oxo-1,4,5,6-tetrahydro-2H-pyrano[3,4-c]isoquinolin-1-yl)-N-methylacetamide;
(S)-2-amino-2-(4-chlorophenyl)-N-((R)-8,9-difluoro-6-oxo-1,4,5,6-tetrahydro-2H-pyrano[3,4-c]isoquinolin-1-yl)-N-methylacetamide;
(R)-2-amino-2-(4-chlorophenyl)-N-((R)-8,9-difluoro-6-oxo-1,4,5,6-tetrahydro-2H-pyrano[3,4-c]isoquinolin-1-yl)-N-methylacetamide;
2-amino-2-(4-chlorophenyl)-N-((S)-8,9-difluoro-6-oxo-1,4,5,6-tetrahydro-2H-pyrano[3,4-c]isoquinolin-1-yl)-N-methylacetamide;
2-amino-2-(4-chlorophenyl)-N-((S)-8,9-difluoro-6-oxo-1,4,5,6-tetrahydro-2H-pyrano[3,4-c]isoquinolin-1-yl)-N-methylacetamide;
(S)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
(R)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
(S)-2-amino-2-(3-chlorophenyl)-N-((S)-8,9-difluoro-6-oxo-1,4,5,6-tetrahydro-2H-pyrano[3,4-c]isoquinolin-1-yl)-N-methylacetamide;
(R)-2-amino-2-(3-chlorophenyl)-N-((S)-8,9-difluoro-6-oxo-1,4,5,6-tetrahydro-2H-pyrano[3,4-c]isoquinolin-1-yl)-N-methylacetamide;
(S)-2-amino-2-(3-chlorophenyl)-N-((R)-8,9-difluoro-6-oxo-1,4,5,6-tetrahydro-2H-pyrano[3,4-c]isoquinolin-1-yl)-N-methylacetamide;
(R)-2-amino-2-(3-chlorophenyl)-N-((R)-8,9-difluoro-6-oxo-1,4,5,6-tetrahydro-2H-pyrano[3,4-c]isoquinolin-1-yl)-N-methylacetamide;
2-amino-2-(3-chlorophenyl)-N-((S)-8,9-difluoro-6-oxo-1,4,5,6-tetrahydro-2H-pyrano[3,4-c]isoquinolin-1-yl)-N-methylacetamide;
2-amino-2-(3-chlorophenyl)-N-((R)-8,9-difluoro-6-oxo-1,4,5,6-tetrahydro-2H-pyrano[3,4-c]isoquinolin-1-yl)-N-methylacetamide;
(S)-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
(R)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
(S)-5-chloro-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
(R)-5-chloro-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
(S)-N-(8,9-difluoro-6-oxo-1,2,3,4,5,6-hexahydrobenzo[c][1,7]naphthyridin-1-yl)-4-(difluoromethyl)-N-methyl-1H-indole-2-carboxamide;
(R)-N-(8,9-difluoro-6-oxo-1,2,3,4,5,6-hexahydrobenzo[c][1,7]naphthyridin-1-yl)-4-(difluoromethyl)-N-methyl-1H-indole-2-carboxamide;
(S)-4-(difluoromethyl)-N-(8-fluoro-6-oxo-1,2,3,4,5,6-hexahydrobenzo[c][1,7]naphthyridin-1-yl)-N-methyl-1H-indole-2-carboxamide;
(R)-4-(difluoromethyl)-N-(8-fluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-1-hydroxy-N-methylcyclohexane-1-carboxamide;
(R)-N-(8,9-difluoro-6-oxo-1,4,5,6-tetrahydro-2H-pyrano[3,4-c]isoquinolin-1-yl)-1-hydroxy-N-methylcyclohexane-1-carboxamide;
(S)-N-(8,9-difluoro-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(S)-5-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-5-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-b]pyridine-2-carboxamide;
(R)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-b]pyridine-2-carboxamide;
(S)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
(R)-5-chloro-N-(8,9-difluoro-6-oxo-1,4,5,6-tetrahydro-2H-pyrano[3,4-c]isoquinolin-1-yl)-N-methyl-6-oxo-1,6-dihydropyridine-3-carboxamide;
(S)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
(R)-5-bromo-N-(8,9-difluoro-6-oxo-1,4,5,6-tetrahydro-2H-pyrano[3,4-c]isoquinolin-1-yl)-N-methyl-6-oxo-1,6-dihydropyridine-2-carboxamide;
(S)-6-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
(R)-6-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[3,2-b]pyridine-2-carboxamide;
(R)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[3,2-b]pyridine-2-carboxamide;
(S)-N-(8,9-difluoro-6-oxo-1,2,3,4,5,6-hexahydrobenzo[c][1,7]naphthyridin-1-yl)-6-(difluoromethyl)-5-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-6-(difluoromethyl)-5-fluoro-N-methyl-1H-indole-2-carboxamide;
(S)-N-(8,9-difluoro-6-oxo-1,2,3,4,5,6-hexahydrobenzo[c][1,7]naphthyridin-1-yl)-6-fluoro-N-methylindolizine-2-carboxamide;
(R)-N-(8,9-difluoro-6-oxo-1,2,3,4,5,6-hexahydrobenzo[c][1,7]naphthyridin-1-yl)-6-fluoro-N-methylindolizine-2-carboxamide;
(S)-N-(8,9-difluoro-6-oxo-1,4,5,6-tetrahydro-2H-pyrano[3,4-c]isoquinolin-1-yl)-6-fluoro-N-methylindolizine-2-carboxamide;
(R)-N-(8,9-difluoro-6-oxo-1,4,5,6-tetrahydro-2H-pyrano[3,4-c]isoquinolin-1-yl)-6-fluoro-N-methylindolizine-2-carboxamide;
(S)-N-(8,9-difluoro-6-oxo-1,4,5,6-tetrahydro-2H-pyrano[3,4-c]isoquinolin-1-yl)-N-methyl-2-(trifluoromethyl)-1H-imidazole-5-carboxamide;
(R)-N-(8,9-difluoro-6-oxo-1,4,5,6-tetrahydro-2H-pyrano[3,4-c]isoquinolin-1-yl)-N-methyl-2-(trifluoromethyl)-1H-imidazole-5-carboxamide;
(S)-N-(8,9-difluoro-6-oxo-1,4,5,6-tetrahydro-2H-pyrano[3,4-c]isoquinolin-1-yl)-5-fluoro-N-methyl-6-oxo-1,6-dihydropyridine-3-carboxamide;
(R)-N-(8,9-difluoro-6-oxo-1,4,5,6-tetrahydro-2H-pyrano[3,4-c]isoquinolin-1-yl)-5-fluoro-N-methyl-6-oxo-1,6-dihydropyridine-3-carboxamide;
(S)-N-(8,9-difluoro-6-oxo-1,4,5,6-tetrahydro-2H-pyrano[3,4-c]isoquinolin-1-yl)-N-methylindolizine-6-carboxamide;
(R)-N-(8,9-difluoro-6-oxo-1,4,5,6-tetrahydro-2H-pyrano[3,4-c]isoquinolin-1-yl)-N-methylindolizine-6-carboxamide;
(S)-N-(8,9-difluoro-6-oxo-1,4,5,6-tetrahydro-2H-pyrano[3,4-c]isoquinolin-1-yl)-N-methylindolizine-7-carboxamide;
(R)-N-(8,9-difluoro-6-oxo-1,4,5,6-tetrahydro-2H-pyrano[3,4-c]isoquinolin-1-yl)-N-methylindolizine-7-carboxamide;
(S)-N-(8,9-difluoro-6-oxo-1,4,5,6-tetrahydro-2H-pyrano[3,4-c]isoquinolin-1-yl)-2-hydroxy-N-methyl-2,2-diphenylacetamide;
(R)-N-(8,9-difluoro-6-oxo-1,4,5,6-tetrahydro-2H-pyrano[3,4-c]isoquinolin-1-yl)-2-hydroxy-N-methyl-2,2-diphenylacetamide;
(S)-N-(8,9-difluoro-6-oxo-1,4,5,6-tetrahydro-2H-pyrano[3,4-c]isoquinolin-1-yl)-4-(difluoromethyl)-6-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4-(difluoromethyl)-6-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-7-fluoro-N-methylindolizine-2-carboxamide;
(R)-N-(8,9-difluoro-6-oxo-1,4,5,6-tetrahydro-2H-pyrano[3,4-c]isoquinolin-1-yl)-7-fluoro-N-methylindolizine-2-carboxamide;
(S)-N-(8,9-difluoro-6-oxo-1,4,5,6-tetrahydro-2H-pyrano[3,4-c]isoquinolin-1-yl)-5-fluoro-N-methyl-1H-pyrrolo[3,2-b]pyridine-2-carboxamide;
(R)-N-(8,9-difluoro-6-oxo-1,4,5,6-tetrahydro-2H-pyrano[3,4-c]isoquinolin-1-yl)-5-fluoro-N-methyl-1H-pyrrolo[3,2-b]pyridine-2-carboxamide;
(S)-N-(8,9-difluoro-6-oxo-1,4,5,6-tetrahydro-2H-pyrano[3,4-c]isoquinolin-1-yl)-5,6-difluoro-N-methyl-2,3-dihydro-1H-indene-2-carboxamide;
(R)-N-(8,9-difluoro-6-oxo-1,4,5,6-tetrahydro-2H-pyrano[3,4-c]isoquinolin-1-yl)-5,6-difluoro-N-methyl-2,3-dihydro-1H-indene-2-carboxamide;
(S)-N-(8,9-difluoro-6-oxo-1,4,5,6-tetrahydro-2H-pyrano[3,4-c]isoquinolin-1-yl)-1-(4-fluorophenyl)-N-methyl-1H-pyrazole-4-carboxamide;
(R)-N-(8,9-difluoro-6-oxo-1,4,5,6-tetrahydro-2H-pyrano[3,4-c]isoquinolin-1-yl)-1-(4-fluorophenyl)-N-methyl-1H-pyrazole-4-carboxamide;
(S)-N-(8,9-difluoro-6-oxo-1,4,5,6-tetrahydro-2H-pyrano[3,4-c]isoquinolin-1-yl)-3-(4-fluorophenyl)-N-methylisoxazole-5-carboxamide;
(R)-N-(8,9-difluoro-6-oxo-1,4,5,6-tetrahydro-2H-pyrano[3,4-c]isoquinolin-1-yl)-3-(4-fluorophenyl)-N-methylisoxazole-5-carboxamide;
(S)-N-(8,9-difluoro-6-oxo-1,4,5,6-tetrahydro-2H-pyrano[3,4-c]isoquinolin-1-yl)-4-(3-fluorophenoxy)-N-methylbenzamide;
(R)-N-(8,9-difluoro-6-oxo-1,4,5,6-tetrahydro-2H-pyrano[3,4-c]isoquinolin-1-yl)-4-(3-fluorophenoxy)-N-methylbenzamide;
(S)-4-(difluoromethyl)-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-(difluoromethyl)-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-5-methyl-6-oxo-1,4,5,6-tetrahydro-2H-pyrano[3,4-c]isoquinolin-1-yl)-5,6-difluoro-N-methyl-1H-indole-2-carboxamide;
(R)-N-(8,9-difluoro-5-methyl-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-methoxy-1,4-dihydro-2H-pyrano[3,4-c]isoquinolin-1-yl)-5,6-difluoro-N-methyl-1H-indole-2-carboxamide;
(R)-N-(8,9-difluoro-6-methoxy-1,4-dihydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-5,6,7,8-tetrahydroindolizine-2-carboxamide;
(R)-N-(8,9-difluoro-6-oxo-1,4,5,6-tetrahydro-2H-pyrano[3,4-c]isoquinolin-1-yl)-N-methyl-5,6,7,8-tetrahydroindolizine-2-carboxamide;
(S)-4-bromo-N-(8,9-difluoro-6-oxo-1,4,5,6-tetrahydro-2H-pyrano[3,4-c]isoquinolin-1-yl)-2,3-difluoro-N-methylbenzamide;
(R)-4-bromo-N-(8,9-difluoro-6-oxo-1,4,5,6-tetrahydro-2H-pyrano[3,4-c]isoquinolin-1-yl)-2,3-difluoro-N-methylbenzamide;
(S)-4-bromo-N-(8,9-difluoro-6-oxo-1,4,5,6-tetrahydro-2H-pyrano[3,4-c]isoquinolin-1-yl)-2,5-difluoro-N-methylbenzamide;
(R)-4-bromo-N-(8,9-difluoro-6-oxo-1,4,5,6-tetrahydro-2H-pyrano[3,4-c]isoquinolin-1-yl)-2,5-difluoro-N-methylbenzamide;
(S)-4-bromo-N-(8,9-difluoro-6-oxo-1,4,5,6-tetrahydro-2H-pyrano[3,4-c]isoquinolin-1-yl)-2,6-difluoro-N-methylbenzamide;
(R)-4-bromo-N-(8,9-difluoro-6-oxo-1,4,5,6-tetrahydro-2H-pyrano[3,4-c]isoquinolin-1-yl)-2,6-difluoro-N-methylbenzamide;
(S)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-2-carboxamide;
(R)-N-(8,9-difluoro-6-oxo-1,4,5,6-tetrahydro-2H-pyrano[3,4-c]isoquinolin-1-yl)-N-methyl-6-oxo-5-(trifluoromethyl)-1,6-dihydropyridine-2-carboxamide;
(S)-N-(8,9-difluoro-6-oxo-1,4,5,6-tetrahydro-2H-pyrano[3,4-c]isoquinolin-1-yl)-N-methylindolizine-3-carboxamide;
(R)-N-(8,9-difluoro-6-oxo-1,4,5,6-tetrahydro-2H-pyrano[3,4-c]isoquinolin-1-yl)-N-methylindolizine-3-carboxamide;
(S)-N-(8,9-difluoro-6-oxo-1,4,5,6-tetrahydro-2H-pyrano[3,4-c]isoquinolin-1-yl)-4-(4-fluorophenoxy)-N-methylbenzamide;
(R)-N-(8,9-difluoro-6-oxo-1,4,5,6-tetrahydro-2H-pyrano[3,4-c]isoquinolin-1-yl)-4-(4-fluorophenoxy)-N-methylbenzamide;
(S)-N-(8,9-difluoro-6-oxo-1,4,5,6-tetrahydro-2H-pyrano[3,4-c]isoquinolin-1-yl)-4′-fluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-4′-fluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-c]pyridine-2-carboxamide;
(R)-N-(8,9-difluoro-6-oxo-1,4,5,6-tetrahydro-2H-pyrano[3,4-c]isoquinolin-1-yl)-N-methyl-5-(trifluoromethyl)-1H-pyrrolo[2,3-c]pyridine-2-carboxamide;
(S)-2-(3-bromophenyl)-N-(8,9-difluoro-6-oxo-1,4,5,6-tetrahydro-2H-pyrano[3,4-c]isoquinolin-1-yl)-2,2-difluoro-N-methylacetamide;
(R)-2-(3-bromophenyl)-N-(8,9-difluoro-6-oxo-1,4,5,6-tetrahydro-2H-pyrano[3,4-c]isoquinolin-1-yl)-2,2-difluoro-N-methylacetamide;
(S)-2-(4-bromophenyl)-N-(8,9-difluoro-6-oxo-1,4,5,6-tetrahydro-2H-pyrano[3,4-c]isoquinolin-1-yl)-2,2-difluoro-N-methylacetamide;
(R)-2-(4-bromophenyl)-N-(8,9-difluoro-6-oxo-1,4,5,6-tetrahydro-2H-pyrano[3,4-c]isoquinolin-1-yl)-2,2-difluoro-N-methylacetamide;
(S)-N-(8,9-difluoro-6-oxo-1,4,5,6-tetrahydro-2H-pyrano[3,4-c]isoquinolin-1-yl)-4-(difluoromethyl)-5-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4-(difluoromethyl)-5-fluoro-N-methyl-1H-indole-2-carboxamide;
(S)-4-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-(difluoromethyl)-5-fluoro-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,2,3,4,5,6-hexahydrobenzo[c][1,7]naphthyridin-1-yl)-4-(difluoromethyl)-6-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-4-(difluoromethyl)-6-fluoro-N-methyl-1H-indole-2-carboxamide;
(S)-4-(difluoromethyl)-6-fluoro-N-(8-fluoro-6-oxo-1,2,3,4,5,6-hexahydrobenzo[c][1,7]naphthyridin-1-yl)-N-methyl-1H-indole-2-carboxamide;
(R)-4-(difluoromethyl)-6-fluoro-N-(8-fluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methylindolizine-1-carboxamide;
(R)-N-(8,9-difluoro-6-oxo-1,4,5,6-tetrahydro-2H-pyrano[3,4-c]isoquinolin-1-yl)-N-methylindolizine-1-carboxamide;
(S)-N-(8,9-difluoro-6-oxo-1,4,5,6-tetrahydro-2H-pyrano[3,4-c]isoquinolin-1-yl)-2,4′-difluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-2,4′-difluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-5-(4-fluorophenyl)-N-methylisoxazole-3-carboxamide;
(R)-N-(8,9-difluoro-6-oxo-1,4,5,6-tetrahydro-2H-pyrano[3,4-c]isoquinolin-1-yl)-5-(4-fluorophenyl)-N-methylisoxazole-3-carboxamide;
(S)-N-(8,9-difluoro-6-oxo-1,2,3,4,5,6-hexahydrobenzo[c][1,7]naphthyridin-1-yl)-4-(difluoromethyl)-5-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-4-(difluoromethyl)-5-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-5-(difluoromethyl)-N-methylindolizine-2-carboxamide;
(R)-N-(8,9-difluoro-6-oxo-1,4,5,6-tetrahydro-2H-pyrano[3,4-c]isoquinolin-1-yl)-5-(difluoromethyl)-N-methylindolizine-2-carboxamide;
(S)-5-bromo-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
(R)-5-bromo-N-(8,9-difluoro-6-oxo-1,4,5,6-tetrahydro-2H-pyrano[3,4-c]isoquinolin-1-yl)-N-methyl-1H-pyrrolo[3,2-b]pyridine-2-carboxamide;
N-((S)-8,9-difluoro-6-oxo-1,4,5,6-tetrahydro-2H-pyrano[3,4-c]isoquinolin-1-yl)-N-methylbicyclo[4.2.0]octa-1(6),2,4-triene-7-carboxamide;
N-((R)-8,9-difluoro-6-oxo-1,4,5,6-tetrahydro-2H-pyrano[3,4-c]isoquinolin-1-yl)-N-methylbicyclo[4.2.0]octa-1(6),2,4-triene-7-carboxamide;
(S)-4-(difluoromethyl)-5-fluoro-N-(8-fluoro-6-oxo-1,2,3,4,5,6-hexahydrobenzo[c][1,7]naphthyridin-1-yl)-N-methyl-1H-indole-2-carboxamide;
(R)-4-(difluoromethyl)-5-fluoro-N-(8-fluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-2-hydroxy-N-methyl-2,3-dihydro-1H-indene-2-carboxamide;
(R)-N-(8,9-difluoro-6-oxo-1,4,5,6-tetrahydro-2H-pyrano[3,4-c]isoquinolin-1-yl)-2-hydroxy-N-methyl-2,3-dihydro-1H-indene-2-carboxamide;
(S)-N-(8,9-difluoro-6-oxo-1,4,5,6-tetrahydro-2H-pyrano[3,4-c]isoquinolin-1-yl)-5,6-difluoro-2-hydroxy-N-methyl-2,3-dihydro-1H-indene-2-carboxamide;
(R)-N-(8,9-difluoro-6-oxo-1,4,5,6-tetrahydro-2H-pyrano[3,4-c]isoquinolin-1-yl)-5,6-difluoro-2-hydroxy-N-methyl-2,3-dihydro-1H-indene-2-carboxamide;
(S)-N-(8,9-difluoro-6-oxo-1,4,5,6-tetrahydro-2H-pyrano[3,4-c]isoquinolin-1-yl)-N,2-dimethylindolizine-6-carboxamide;
(R)-N-(8,9-difluoro-6-oxo-1,4,5,6-tetrahydro-2H-pyrano[3,4-c]isoquinolin-1-yl)-N,2-dimethylindolizine-6-carboxamide;
(S)-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
N-((S)-8,9-difluoro-6-oxo-1,4,5,6-tetrahydro-2H-pyrano[3,4-c]isoquinolin-1-yl)-4-fluoro-N-methylbicyclo[4.2.0]octa-1(6),2,4-triene-7-carboxamide;
N-((R)-8,9-difluoro-6-oxo-1,4,5,6-tetrahydro-2H-pyrano[3,4-c]isoquinolin-1-yl)-4-fluoro-N-methylbicyclo[4.2.0]octa-1(6),2,4-triene-7-carboxamide;
(S)-N-(6-(2-aminoethoxy)-8,9-difluoro-1,4-dihydro-2H-pyrano[3,4-c]isoquinolin-1-yl)-5,6-difluoro-N-methyl-1H-indole-2-carboxamide;
(R)-N-(6-(2-aminoethoxy)-8,9-difluoro-1,4-dihydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-1-(thiophen-3-yl)azetidine-3-carboxamide;
(R)-N-(8,9-difluoro-6-oxo-1,4,5,6-tetrahydro-2H-pyrano[3,4-c]isoquinolin-1-yl)-N-methyl-1-(thiophen-3-yl)azetidine-3-carboxamide;
(S)-N-(8,9-difluoro-6-oxo-1,4,5,6-tetrahydro-2H-pyrano[3,4-c]isoquinolin-1-yl)-3′-fluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-3′-fluoro-N-methyl-[1,1′-biphenyl]-4-carboxamide;
(S)-1-(4-bromothiophen-3-yl)-N-(8,9-difluoro-6-oxo-1,4,5,6-tetrahydro-2H-pyrano[3,4-c]isoquinolin-1-yl)-N-methylazetidine-3-carboxamide;
(R)-1-(4-bromothiophen-3-yl)-N-(8,9-difluoro-6-oxo-1,4,5,6-tetrahydro-2H-pyrano[3,4-c]isoquinolin-1-yl)-N-methylazetidine-3-carboxamide;
(S)-N-(8,9-difluoro-6-oxo-1,4,5,6-tetrahydro-2H-pyrano[3,4-c]isoquinolin-1-yl)-2′-fluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-2′-fluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-3′,5′-difluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-3′,5′-difluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-2,3′,5′-trifluoro-N-methyl-[1,1′-biphenyl]-4-carboxamide;
(R)-N-(8,9-difluoro-6-oxo-1,4,5,6-tetrahydro-2H-pyrano[3,4-c]isoquinolin-1-yl)-2,3′,5′-trifluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-N-methyl-[1,1′-biphenyl]-3-carboxamide;
(R)-N-(8,9-difluoro-6-oxo-1,4,5,6-tetrahydro-2H-pyrano[3,4-c]isoquinolin-1-yl)-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-4′-fluoro-N-methyl-[1,1′-biphenyl]-3-carboxamide;
(R)-N-(8,9-difluoro-6-oxo-1,4,5,6-tetrahydro-2H-pyrano[3,4-c]isoquinolin-1-yl)-4′-fluoro-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-3′-fluoro-N-methyl-[1,1′-biphenyl]-3-carboxamide;
(R)-N-(8,9-difluoro-6-oxo-1,4,5,6-tetrahydro-2H-pyrano[3,4-c]isoquinolin-1-yl)-3′-fluoro-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-3′,5-difluoro-N-methyl-[1,1′-biphenyl]-3-carboxamide;
(R)-N-(8,9-difluoro-6-oxo-1,4,5,6-tetrahydro-2H-pyrano[3,4-c]isoquinolin-1-yl)-3′,5-difluoro-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-N-methyl-3-phenoxybenzamide;
(R)-N-(8,9-difluoro-6-oxo-1,4,5,6-tetrahydro-2H-pyrano[3,4-c]isoquinolin-1-yl)-N-methyl-3-phenoxybenzamide;
(S)-N-(8,9-difluoro-6-oxo-1,4,5,6-tetrahydro-2H-pyrano[3,4-c]isoquinolin-1-yl)-3-(4-fluorophenoxy)-N-methylbenzamide;
(R)-N-(8,9-difluoro-6-oxo-1,4,5,6-tetrahydro-2H-pyrano[3,4-c]isoquinolin-1-yl)-3-(4-fluorophenoxy)-N-methylbenzamide;
(S)-N-(8,9-difluoro-6-oxo-1,4,5,6-tetrahydro-2H-pyrano[3,4-c]isoquinolin-1-yl)-N-methyl-6H-thieno[2,3-b]pyrrole-5-carboxamide;
(R)-N-(8,9-difluoro-6-oxo-1,4,5,6-tetrahydro-2H-pyrano[3,4-c]isoquinolin-1-yl)-N-methyl-6H-thieno[2,3-b]pyrrole-5-carboxamide;
(S)-N-(8,9-difluoro-6-oxo-1,4,5,6-tetrahydro-2H-pyrano[3,4-c]isoquinolin-1-yl)-N-methyl-4-(methylsulfonyl)benzamide;
(R)-N-(8,9-difluoro-6-oxo-1,4,5,6-tetrahydro-2H-pyrano[3,4-c]isoquinolin-1-yl)-N-methyl-4-(methylsulfonyl)benzamide;
(S)-N-(8,9-difluoro-6-oxo-1,4,5,6-tetrahydro-2H-pyrano[3,4-c]isoquinolin-1-yl)-N-methyl-3-(N-methylsulfamoyl)benzamide;
(R)-N-(8,9-difluoro-6-oxo-1,4,5,6-tetrahydro-2H-pyrano[3,4-c]isoquinolin-1-yl)-N-methyl-3-(N-methylsulfamoyl)benzamide;
(S)-N-(8,9-difluoro-6-oxo-1,4,5,6-tetrahydro-2H-pyrano[3,4-c]isoquinolin-1-yl)-N-methyl-3-(methylsulfonamido)benzamide;
(R)-N-(8,9-difluoro-6-oxo-1,4,5,6-tetrahydro-2H-pyrano[3,4-c]isoquinolin-1-yl)-N-methyl-3-(methylsulfonamido)benzamide;
(S)-N-(8,9-difluoro-6-oxo-1,4,5,6-tetrahydro-2H-pyrano[3,4-c]isoquinolin-1-yl)-N-methyl-3-(methylsulfonyl)benzamide;
(R)-N-(8,9-difluoro-6-oxo-1,4,5,6-tetrahydro-2H-pyrano[3,4-c]isoquinolin-1-yl)-N-methyl-3-(methylsulfonyl)benzamide;
(S)-N-(8,9-difluoro-6-oxo-1,4,5,6-tetrahydro-2H-pyrano[3,4-c]isoquinolin-1-yl)-N-methylimidazo[1,5-a]pyridine-6-carboxamide;
(R)-N-(8,9-difluoro-6-oxo-1,4,5,6-tetrahydro-2H-pyrano[3,4-c]isoquinolin-1-yl)-N-methylimidazo[1,5-a]pyridine-6-carboxamide;
(1S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1S,4S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1S,4R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1R,4R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(1R,4S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl isobutyrate;
(S)-N-(8,9-difluoro-6-oxo-1,2,3,4,5,6-hexahydrobenzo[c][1,7]naphthyridin-1-yl)-3′-fluoro-N-methyl-[1,1′-biphenyl]-4-carboxamide;
(R)-N-(8,9-difluoro-6-oxo-1,2,3,4,5,6-hexahydrobenzo[c][1,7]naphthyridin-1-yl)-3′-fluoro-N-methyl-[1,1′-biphenyl]-4-carboxamide;
(S)-N-(8,9-difluoro-6-oxo-1,4,5,6-tetrahydro-2H-pyrano[3,4-c]isoquinolin-1-yl)-3-fluoro-N-methyl-5-phenoxybenzamide;
(R)-N-(8,9-difluoro-6-oxo-1,4,5,6-tetrahydro-2H-pyrano[3,4-c]isoquinolin-1-yl)-3-fluoro-N-methyl-5-phenoxybenzamide;
(S)-N-(8,9-difluoro-6-oxo-1,2,3,4,5,6-hexahydrobenzo[c][1,7]naphthyridin-1-yl)-3′-fluoro-N-methyl-[1,1′-biphenyl]-3-carboxamide;
(R)-N-(8,9-difluoro-6-oxo-1,2,3,4,5,6-hexahydrobenzo[c][1,7]naphthyridin-1-yl)-3′-fluoro-N-methyl-[1,1′-biphenyl]-3-carboxamide;
(S)-N-(8,9-difluoro-6-oxo-1,4,5,6-tetrahydro-2H-pyrano[3,4-c]isoquinolin-1-yl)-N-methylimidazo[1,2-a]pyridine-6-carboxamide;
(R)-N-(8,9-difluoro-6-oxo-1,4,5,6-tetrahydro-2H-pyrano[3,4-c]isoquinolin-1-yl)-N-methylimidazo[1,2-a]pyridine-6-carboxamide;
(S)-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(R)-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(S)-6-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(R)-6-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(S)-N-(8,9-difluoro-6-oxo-1,4,5,6-tetrahydro-2H-pyrano[3,4-c]isoquinolin-1-yl)-5-fluoro-N-methyl-1H-pyrrolo[2,3-c]pyridine-2-carboxamide;
(R)-N-(8,9-difluoro-6-oxo-1,4,5,6-tetrahydro-2H-pyrano[3,4-c]isoquinolin-1-yl)-5-fluoro-N-methyl-1H-pyrrolo[2,3-c]pyridine-2-carboxamide;
(S)-N-(8,9-difluoro-6-oxo-1,4,5,6-tetrahydro-2H-pyrano[3,4-c]isoquinolin-1-yl)-N-methyl-2-(trifluoromethyl)indolizine-6-carboxamide;
(R)-N-(8,9-difluoro-6-oxo-1,4,5,6-tetrahydro-2H-pyrano[3,4-c]isoquinolin-1-yl)-N-methyl-2-(trifluoromethyl)indolizine-6-carboxamide;
(S)-4-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-4-bromo-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-N-(8,9-difluoro-6-oxo-1,4,5,6-tetrahydro-2H-pyrano[3,4-c]isoquinolin-1-yl)-2-(difluoromethyl)-N-methylisonicotinamide;
(R)-N-(8,9-difluoro-6-oxo-1,4,5,6-tetrahydro-2H-pyrano[3,4-c]isoquinolin-1-yl)-2-(difluoromethyl)-N-methylisonicotinamide;
(S)-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
(R)-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
(S)-N-(8,9-difluoro-5-(2-hydroxyethyl)-6-oxo-1,4,5,6-tetrahydro-2H-pyrano[3,4-c]isoquinolin-1-yl)-5,6-difluoro-N-methyl-1H-indole-2-carboxamide;
(R)-N-(8,9-difluoro-5-(2-hydroxyethyl)-6-oxo-1,4,5,6-tetrahydro-2H-pyrano[3,4-c]isoquinolin-1-yl)-5,6-difluoro-N-methyl-1H-indole-2-carboxamide;
(S)-N-(5-(2-aminoethyl)-8,9-difluoro-6-oxo-1,4,5,6-tetrahydro-2H-pyrano[3,4-c]isoquinolin-1-yl)-5,6-difluoro-N-methyl-1H-indole-2-carboxamide;
(R)-N-(5-(2-aminoethyl)-8,9-difluoro-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-(2-hydroxyethoxy)-1,4-dihydro-2H-pyrano[3,4-c]isoquinolin-1-yl)-5,6-difluoro-N-methyl-1H-indole-2-carboxamide;
(R)-N-(8,9-difluoro-6-(2-hydroxyethoxy)-1,4-dihydro-2H-pyrano[3,4-c]isoquinolin-1-yl)-5,6-difluoro-N-methyl-1H-indole-2-carboxamide;
(S)-N-(8,9-difluoro-6-oxo-1,2,3,4,5,6-hexahydrobenzo[c][1,7]naphthyridin-1-yl)-N-methyl-3-(N-methylsulfamoyl)benzamide;
(R)-N-(8,9-difluoro-6-oxo-1,2,3,4,5,6-hexahydrobenzo[c][1,7]naphthyridin-1-yl)-N-methyl-3-(N-methylsulfamoyl)benzamide;
N-((1S)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1S,4R)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1S,4S)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1R)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1R,4R)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
N-((1R,4S)-8,9-difluoro-4-methoxy-6-oxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-4,6-dioxo-1,4,5,6-tetrahydro-2H-pyrano[3,4-c]isoquinolin-1-yl)-5,6-difluoro-N-methyl-1H-indole-2-carboxamide;
(R)-N-(8,9-difluoro-4,6-dioxo-1,4,5,6-tetrahydro-2H-pyrano[3,4-c]isoquinolin-1-yl)-5,6-difluoro-N-methyl-1H-indole-2-carboxamide;
(S)-N-(8,9-difluoro-6-oxo-1,4,5,6-tetrahydro-2H-pyrano[3,4-c]isoquinolin-1-yl)-3-(3-fluorophenoxy)-N-methylbenzamide;
(R)-N-(8,9-difluoro-6-oxo-1,4,5,6-tetrahydro-2H-pyrano[3,4-c]isoquinolin-1-yl)-3-(3-fluorophenoxy)-N-methylbenzamide;
(S)-N-(8,9-difluoro-6-oxo-1,4,5,6-tetrahydro-2H-pyrano[3,4-c]isoquinolin-1-yl)-5-methoxy-N-methyl-1H-indole-2-carboxamide;
(R)-N-(8,9-difluoro-6-oxo-1,4,5,6-tetrahydro-2H-pyrano[3,4-c]isoquinolin-1-yl)-5-methoxy-N-methyl-1H-indole-2-carboxamide;
(S)-5-chloro-N-(8,9-difluoro-6-oxo-1,4,5,6-tetrahydro-2H-pyrano[3,4-c]isoquinolin-1-yl)-N-methyl-1H-indole-2-carboxamide;
(R)-5-chloro-N-(8,9-difluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N,5-dimethyl-1H-indole-2-carboxamide;
(R)-N-(8,9-difluoro-6-oxo-1,4,5,6-tetrahydro-2H-pyrano[3,4-c]isoquinolin-1-yl)-N,5-dimethyl-1H-indole-2-carboxamide;
(S)-N-(8,9-difluoro-6-oxo-1,4,5,6-tetrahydro-2H-pyrano[3,4-c]isoquinolin-1-yl)-N-methyl-5-(trifluoromethoxy)-1H-indole-2-carboxamide;
(R)-N-(8,9-difluoro-6-oxo-1,4,5,6-tetrahydro-2H-pyrano[3,4-c]isoquinolin-1-yl)-N-methyl-5-(trifluoromethoxy)-1H-indole-2-carboxamide;
(S)-N-(8,9-difluoro-6-oxo-1,4,5,6-tetrahydro-2H-pyrano[3,4-c]isoquinolin-1-yl)-5-ethyl-N-methyl-1H-indole-2-carboxamide;
(R)-N-(8,9-difluoro-6-oxo-1,4,5,6-tetrahydro-2H-pyrano[3,4-c]isoquinolin-1-yl)-5-ethyl-N-methyl-1H-indole-2-carboxamide;
(S)-N-(8,9-difluoro-6-oxo-1,4,5,6-tetrahydro-2H-pyrano[3,4-c]isoquinolin-1-yl)-4-methoxy-N-methyl-1H-indole-2-carboxamide;
(R)-N-(8,9-difluoro-6-oxo-1,4,5,6-tetrahydro-2H-pyrano[3,4-c]isoquinolin-1-yl)-4-methoxy-N-methyl-1H-indole-2-carboxamide;
(S)-N-(8,9-difluoro-6-oxo-1,4,5,6-tetrahydro-2H-pyrano[3,4-c]isoquinolin-1-yl)-N,4-dimethyl-1H-indole-2-carboxamide;
(R)-N-(8,9-difluoro-6-oxo-1,4,5,6-tetrahydro-2H-pyrano[3,4-c]isoquinolin-1-yl)-N,4-dimethyl-1H-indole-2-carboxamide;
(1S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1S,4S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1S,4R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1R,4S)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(1R,4R)-1-(5,6-difluoro-N-methyl-1H-indole-2-carboxamido)-8,9-difluoro-6-oxo-1,4,5,6-tetrahydro-2H-pyrano[3,4-c]isoquinolin-4-yl 2-ethylbutanoate;
(S)-2-chloro-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
(R)-2-chloro-N-(8,9-difluoro-6-oxo-1,4,5,6-tetrahydro-2H-pyrano[3,4-c]isoquinolin-1-yl)-N-methyl-4H-furo[3,2-b]pyrrole-5-carboxamide;
(S)-N-(8,9-difluoro-6-(methylamino)-1,4-dihydro-2H-pyrano[3,4-c]isoquinolin-1-yl)-5,6-difluoro-N-methyl-1H-indole-2-carboxamide;
(R)-N-(8,9-difluoro-6-(methylamino)-1,4-dihydro-2H-pyrano[3,4-c]isoquinolin-1-yl)-5,6-difluoro-N-methyl-1H-indole-2-carboxamide;
(S)-N-(8,9-difluoro-6-((2-hydroxyethyl)amino)-1,4-dihydro-2H-pyrano[3,4-c]isoquinolin-1-yl)-5,6-difluoro-N-methyl-1H-indole-2-carboxamide;
(R)-N-(8,9-difluoro-6-((2-hydroxyethyl)amino)-1,4-dihydro-2H-pyrano[3,4-c]isoquinolin-1-yl)-5,6-difluoro-N-methyl-1H-indole-2-carboxamide;
(S)-N-(8,9-difluoro-6-oxo-1,2,3,4,5,6-hexahydrobenzo[c][1,7]naphthyridin-1-yl)-3-(3-fluorophenoxy)-N-methylbenzamide;
(R)-N-(8,9-difluoro-6-oxo-1,2,3,4,5,6-hexahydrobenzo[c][1,7]naphthyridin-1-yl)-3-(3-fluorophenoxy)-N-methylbenzamide;
(S)-4-cyano-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(R)-4-cyano-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-4-ethyl-6-fluoro-N-methyl-1H-indole-2-carboxamide;
(R)-N-(8,9-difluoro-6-oxo-1,4,5,6-tetrahydro-2H-pyrano[3,4-c]isoquinolin-1-yl)-4-ethyl-6-fluoro-N-methyl-1H-indole-2-carboxamide;
(S)-4-chloro-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(R)-4-chloro-N-(8,9-difluoro-6-oxo-1,4,5,6-tetrahydro-2H-pyrano[3,4-c]isoquinolin-1-yl)-6-fluoro-N-methyl-1H-indole-2-carboxamide;
(S)-N-(8,9-difluoro-6-oxo-1,4,5,6-tetrahydro-2H-pyrano[3,4-c]isoquinolin-1-yl)-6-fluoro-N,4-dimethyl-1H-indole-2-carboxamide;
(R)-N-(8,9-difluoro-6-oxo-1,4,5,6-tetrahydro-2H-pyrano[3,4-c]isoquinolin-1-yl)-6-fluoro-N,4-dimethyl-1H-indole-2-carboxamide;
(S)-5-cyano-N-(8,9-difluoro-6-oxo-1,4,5,6-tetrahydro-2H-pyrano[3,4-c]isoquinolin-1-yl)-N-methyl-1H-indole-2-carboxamide;
(R)-5-cyano-N-(8,9-difluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methyl-5-(methylsulfonyl)-1H-indole-2-carboxamide;
(R)-N-(8,9-difluoro-6-oxo-1,4,5,6-tetrahydro-2H-pyrano[3,4-c]isoquinolin-1-yl)-N-methyl-5-(methylsulfonyl)-1H-indole-2-carboxamide;
(S)-4-chloro-N-(8,9-difluoro-6-oxo-1,4,5,6-tetrahydro-2H-pyrano[3,4-c]isoquinolin-1-yl)-N-methyl-1H-indole-2-carboxamide;
(R)-4-chloro-N-(8,9-difluoro-6-oxo-1,4,5,6-tetrahydro-2H-pyrano[3,4-c]isoquinolin-1-yl)-N-methyl-1H-indole-2-carboxamide;
(S)-6-bromo-N-(8,9-difluoro-6-oxo-1,4,5,6-tetrahydro-2H-pyrano[3,4-c]isoquinolin-1-yl)-N-methylindolizine-2-carboxamide;
(R)-6-bromo-N-(8,9-difluoro-6-oxo-1,4,5,6-tetrahydro-2H-pyrano[3,4-c]isoquinolin-1-yl)-N-methylindolizine-2-carboxamide;
(S)-2-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-2-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-3-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-3-chloro-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-N-(6-((2-aminoethyl)amino)-8,9-difluoro-1,4-dihydro-2H-pyrano[3,4-c]isoquinolin-1-yl)-5,6-difluoro-N-methyl-1H-indole-2-carboxamide;
(R)-N-(6-((2-aminoethyl)amino)-8,9-difluoro-1,4-dihydro-2H-pyrano[3,4-c]isoquinolin-1-yl)-5,6-difluoro-N-methyl-1H-indole-2-carboxamide;
N-((S)-8,9-difluoro-6-oxo-1,4,5,6-tetrahydro-2H-pyrano[3,4-c]isoquinolin-1-yl)-6-fluoro-4-(1-hydroxyethyl)-N-methyl-1H-indole-2-carboxamide;
N-((R)-8,9-difluoro-6-oxo-1,4,5,6-tetrahydro-2H-pyrano[3,4-c]isoquinolin-1-yl)-6-fluoro-4-(1-hydroxyethyl)-N-methyl-1H-indole-2-carboxamide;
N-((S)-8,9-difluoro-6-oxo-1,4,5,6-tetrahydro-2H-pyrano[3,4-c]isoquinolin-1-yl)-6-fluoro-4-((S)-1-hydroxyethyl)-N-methyl-1H-indole-2-carboxamide;
N-((R)-8,9-difluoro-6-oxo-1,4,5,6-tetrahydro-2H-pyrano[3,4-c]isoquinolin-1-yl)-6-fluoro-4-((S)-1-hydroxyethyl)-N-methyl-1H-indole-2-carboxamide;
N-((S)-8,9-difluoro-6-oxo-1,4,5,6-tetrahydro-2H-pyrano[3,4-c]isoquinolin-1-yl)-6-fluoro-4-((R)-1-hydroxyethyl)-N-methyl-1H-indole-2-carboxamide;
N-((S)-8,9-difluoro-6-oxo-1,4,5,6-tetrahydro-2H-pyrano[3,4-c]isoquinolin-1-yl)-6-fluoro-4-((S)-1-hydroxyethyl)-N-methyl-1H-indole-2-carboxamide;
(S)-N-(8,9-difluoro-6-oxo-1,4,5,6-tetrahydro-2H-pyrano[3,4-c]isoquinolin-1-yl)-6-fluoro-4-(hydroxymethyl)-N-methyl-1H-indole-2-carboxamide;
(R)-N-(8,9-difluoro-6-oxo-1,4,5,6-tetrahydro-2H-pyrano[3,4-c]isoquinolin-1-yl)-6-fluoro-4-(hydroxymethyl)-N-methyl-1H-indole-2-carboxamide;
(S)-7-bromo-N-(8,9-difluoro-6-oxo-1,4,5,6-tetrahydro-2H-pyrano[3,4-c]isoquinolin-1-yl)-N-methylfuro[3,2-c]pyridine-2-carboxamide;
(R)-7-bromo-N-(8,9-difluoro-6-oxo-1,4,5,6-tetrahydro-2H-pyrano[3,4-c]isoquinolin-1-yl)-N-methylfuro[3,2-c]pyridine-2-carboxamide;
(S)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-6-carboxamide;
(R)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-6-carboxamide;
(S)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-6-carboxamide;
(R)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-6-carboxamide;
(S)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
(R)-3-chloro-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
(S)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-5-carboxamide;
(R)-3-chloro-N-(8,9-difluoro-6-oxo-1,4,5,6-tetrahydro-2H-pyrano[3,4-c]isoquinolin-1-yl)-2-(difluoromethyl)-N-methyl-2H-indazole-5-carboxamide;
(S)-3,4-dichloro-N-(8,9-difluoro-6-oxo-1,4,5,6-tetrahydro-2H-pyrano[3,4-c]isoquinolin-1-yl)-N-methylbenzamide;
(R)-3,4-dichloro-N-(8,9-difluoro-6-oxo-1,4,5,6-tetrahydro-2H-pyrano[3,4-c]isoquinolin-1-yl)-N-methylbenzamide;
(S)-N-(8,9-difluoro-6-oxo-1,4,5,6-tetrahydro-2H-pyrano[3,4-c]isoquinolin-1-yl)-6-fluoro-N-methyl-4-(methylsulfonamido)-1H-indole-2-carboxamide;
(R)-N-(8,9-difluoro-6-oxo-1,4,5,6-tetrahydro-2H-pyrano[3,4-c]isoquinolin-1-yl)-6-fluoro-N-methyl-4-(methylsulfonamido)-1H-indole-2-carboxamide;
(S)-N-(8,9-difluoro-6-oxo-1,4,5,6-tetrahydro-2H-pyrano[3,4-c]isoquinolin-1-yl)-N-methyl-4-(trifluoromethoxy)-1H-indole-2-carboxamide;
(R)-N-(8,9-difluoro-6-oxo-1,4,5,6-tetrahydro-2H-pyrano[3,4-c]isoquinolin-1-yl)-N-methyl-4-(trifluoromethoxy)-1H-indole-2-carboxamide;
(S)-N-(8,9-difluoro-6-oxo-1,4,5,6-tetrahydro-2H-pyrano[3,4-c]isoquinolin-1-yl)-6-methoxy-N-methyl-1H-indole-2-carboxamide;
(R)-N-(8,9-difluoro-6-oxo-1,4,5,6-tetrahydro-2H-pyrano[3,4-c]isoquinolin-1-yl)-6-methoxy-N-methyl-1H-indole-2-carboxamide;
(S)-5-bromo-N-(8,9-difluoro-6-oxo-1,4,5,6-tetrahydro-2H-pyrano[3,4-c]isoquinolin-1-yl)-N-methyl-6-(trifluoromethyl)nicotinamide;
(R)-5-bromo-N-(8,9-difluoro-6-oxo-1,4,5,6-tetrahydro-2H-pyrano[3,4-c]isoquinolin-1-yl)-N-methyl-6-(trifluoromethyl)nicotinamide;
(S)-3-chloro-N-(8,9-difluoro-6-oxo-1,4,5,6-tetrahydro-2H-pyrano[3,4-c]isoquinolin-1-yl)-N-methylindolizine-6-carboxamide;
(R)-3-chloro-N-(8,9-difluoro-6-oxo-1,4,5,6-tetrahydro-2H-pyrano[3,4-c]isoquinolin-1-yl)-N-methylindolizine-6-carboxamide;
(S)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-6-carboxamide;
(R)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-6-carboxamide;
(S)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-5-carboxamide;
(R)-N-(8,9-difluoro-6-oxo-1,4,5,6-tetrahydro-2H-pyrano[3,4-c]isoquinolin-1-yl)-2-(difluoromethyl)-3-methoxy-N-methyl-2H-indazole-5-carboxamide;
(S)-N-(8,9-difluoro-6-oxo-1,4,5,6-tetrahydro-2H-pyrano[3,4-c]isoquinolin-1-yl)-3-methoxy-N-methyl-1H-indazole-5-carboxamide;
(R)-N-(8,9-difluoro-6-oxo-1,4,5,6-tetrahydro-2H-pyrano[3,4-c]isoquinolin-1-yl)-3-methoxy-N-methyl-1H-indazole-5-carboxamide;
(S)-1-(5-chlorothiophen-3-yl)-N-(8,9-difluoro-6-oxo-1,4,5,6-tetrahydro-2H-pyrano[3,4-c]isoquinolin-1-yl)-N-methylazetidine-3-carboxamide;
(R)-1-(5-chlorothiophen-3-yl)-N-(8,9-difluoro-6-oxo-1,4,5,6-tetrahydro-2H-pyrano[3,4-c]isoquinolin-1-yl)-N-methylazetidine-3-carboxamide;
(S)-7-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-7-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-4-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(R)-4-chloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(S)-3-chloro-N-(8,9-difluoro-6-oxo-1,4,5,6-tetrahydro-2H-pyrano[3,4-c]isoquinolin-1-yl)-N-methylindolizine-7-carboxamide;
(R)-3-chloro-N-(8,9-difluoro-6-oxo-1,4,5,6-tetrahydro-2H-pyrano[3,4-c]isoquinolin-1-yl)-N-methylindolizine-7-carboxamide;
(S)-N-(8,9-difluoro-6-oxo-1,4,5,6-tetrahydro-2H-pyrano[3,4-c]isoquinolin-1-yl)-7-(difluoromethyl)-N-methylindolizine-2-carboxamide;
(R)-N-(8,9-difluoro-6-oxo-1,4,5,6-tetrahydro-2H-pyrano[3,4-c]isoquinolin-1-yl)-7-(difluoromethyl)-N-methylindolizine-2-carboxamide;
(S)-N-(8,9-difluoro-6-oxo-1,4,5,6-tetrahydro-2H-pyrano[3,4-c]isoquinolin-1-yl)-6-(difluoromethyl)-N-methylindolizine-2-carboxamide;
(R)-N-(8,9-difluoro-6-oxo-1,4,5,6-tetrahydro-2H-pyrano[3,4-c]isoquinolin-1-yl)-6-(difluoromethyl)-N-methylindolizine-2-carboxamide;
(S)-di-tert-butyl ((2-((8,9-difluoro-6-oxo-1,4,5,6-tetrahydro-2H-pyrano[3,4-c]isoquinolin-1-yl)(methyl)carbamoyl)-5,6-difluoro-1H-indol-1-yl)methyl)phosphate;
(R)-di-tert-butyl ((2-((8,9-difluoro-6-oxo-1,4,5,6-tetrahydro-2H-pyrano[3,4-c]isoquinolin-1-yl)(methyl)carbamoyl)-5,6-difluoro-1H-indol-1-yl)methyl)phosphate;
(S)-5-chloro-N-(8,9-difluoro-6-oxo-1,4,5,6-tetrahydro-2H-pyrano[3,4-c]isoquinolin-1-yl)-N-methyl-6-(trifluoromethyl)nicotinamide;
(R)-5-chloro-N-(8,9-difluoro-6-oxo-1,4,5,6-tetrahydro-2H-pyrano[3,4-c]isoquinolin-1-yl)-N-methyl-6-(trifluoromethyl)nicotinamide;
(S)-5-chloro-N-(8-fluoro-6-oxo-1,4,5,6-tetrahydro-2H-pyrano[3,4-c]isoquinolin-1-yl)-N-methyl-6-(trifluoromethyl)nicotinamide;
(R)-5-chloro-N-(8-fluoro-6-oxo-1,4,5,6-tetrahydro-2H-pyrano[3,4-c]isoquinolin-1-yl)-N-methyl-6-(trifluoromethyl)nicotinamide;
(S)-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-4-cyano-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-cyano-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-4-bromo-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(R)-4-bromo-5-chloro-N-(8,9-difluoro-6-oxo-1,4,5,6-tetrahydro-2H-pyrano[3,4-c]isoquinolin-1-yl)-N-methyl-1H-pyrrolo[2,3-c]pyridine-2-carboxamide;
(S)-N-(8,9-difluoro-6-oxo-1,2,3,4,5,6-hexahydrobenzo[c][1,7]naphthyridin-1-yl)-7-(difluoromethyl)-N-methylindolizine-2-carboxamide;
(R)-N-(8,9-difluoro-6-oxo-1,2,3,4,5,6-hexahydrobenzo[c][1,7]naphthyridin-1-yl)-7-(difluoromethyl)-N-methylindolizine-2-carboxamide;
(S)-N-(8,9-difluoro-6-oxo-1,2,3,4,5,6-hexahydrobenzo[c][1,7]naphthyridin-1-yl)-6-(difluoromethyl)-N-methylindolizine-2-carboxamide;
(R)-N-(8,9-difluoro-6-oxo-1,2,3,4,5,6-hexahydrobenzo[c][1,7]naphthyridin-1-yl)-6-(difluoromethyl)-N-methylindolizine-2-carboxamide;
(S)-N-(8-fluoro-6-oxo-1,4,5,6-tetrahydro-2H-pyrano[3,4-c]isoquinolin-1-yl)-N-methylindolizine-6-carboxamide;
(R)-N-(8-fluoro-6-oxo-1,4,5,6-tetrahydro-2H-pyrano[3,4-c]isoquinolin-1-yl)-N-methylindolizine-6-carboxamide;
(S)-N-(8,9-difluoro-6-oxo-1,2,3,4,5,6-hexahydrobenzo[c][1,7]naphthyridin-1-yl)-N-methylindolizine-6-carboxamide;
(R)-N-(8,9-difluoro-6-oxo-1,2,3,4,5,6-hexahydrobenzo[c][1,7]naphthyridin-1-yl)-N-methylindolizine-6-carboxamide;
(S)-4-ethyl-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-ethyl-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-5-cyano-N-(8-fluoro-6-oxo-1,4,5,6-tetrahydro-2H-pyrano[3,4-c]isoquinolin-1-yl)-N-methyl-1H-indole-2-carboxamide;
(R)-5-cyano-N-(8-fluoro-6-oxo-1,4,5,6-tetrahydro-2H-pyrano[3,4-c]isoquinolin-1-yl)-N-methyl-1H-indole-2-carboxamide;
(S)-4-cyano-N-(8,9-difluoro-6-oxo-1,2,3,4,5,6-hexahydrobenzo[c][1,7]naphthyridin-1-yl)-6-fluoro-N-methyl-1H-indole-2-carboxamide;
(R)-4-cyano-N-(8,9-difluoro-6-oxo-1,2,3,4,5,6-hexahydrobenzo[c][1,7]naphthyridin-1-yl)-6-fluoro-N-methyl-1H-indole-2-carboxamide;
(S)-N-(8,9-difluoro-6-oxo-1,2,3,4,5,6-hexahydrobenzo[c][1,7]naphthyridin-1-yl)-4-ethyl-6-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-4-ethyl-6-fluoro-N-methyl-1H-indole-2-carboxamide;
(S)-N-(8,9-difluoro-6-oxo-1,2,3,4,5,6-hexahydrobenzo[c][1,7]naphthyridin-1-yl)-N-methyl-5-(methylsulfonyl)-1H-indole-2-carboxamide;
(R)-N-(8,9-difluoro-6-oxo-1,2,3,4,5,6-hexahydrobenzo[c][1,7]naphthyridin-1-yl)-N-methyl-5-(methylsulfonyl)-1H-indole-2-carboxamide;
(S)-5-cyano-N-(8,9-difluoro-6-oxo-1,2,3,4,5,6-hexahydrobenzo[c][1,7]naphthyridin-1-yl)-N-methyl-1H-indole-2-carboxamide;
(R)-5-cyano-N-(8,9-difluoro-6-oxo-1,2,3,4,5,6-hexahydrobenzo[c][1,7]naphthyridin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-8-(difluoromethyl)-N-methylindolizine-2-carboxamide;
(R)-N-(8,9-difluoro-6-oxo-1,4,5,6-tetrahydro-2H-pyrano[3,4-c]isoquinolin-1-yl)-8-(difluoromethyl)-N-methylindolizine-2-carboxamide;
(S)-N-(8,9-difluoro-6-oxo-1,4,5,6-tetrahydro-2H-pyrano[3,4-c]isoquinolin-1-yl)-N-methyl-2,3-dihydro-1H-indene-5-carboxamide;
(R)-N-(8,9-difluoro-6-oxo-1,4,5,6-tetrahydro-2H-pyrano[3,4-c]isoquinolin-1-yl)-N-methyl-2,3-dihydro-1H-indene-5-carboxamide;
(S)-N-(8,9-difluoro-6-oxo-1,4,5,6-tetrahydro-2H-pyrano[3,4-c]isoquinolin-1-yl)-N-methylbenzo[d]oxazole-6-carboxamide;
(R)-N-(8,9-difluoro-6-oxo-1,4,5,6-tetrahydro-2H-pyrano[3,4-c]isoquinolin-1-yl)-N-methylbenzo[d]oxazole-6-carboxamide;
(S)-4,6-dichloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
(R)-4,6-dichloro-N-(8,9-difluoro-6-oxo-1,4,5,6-tetrahydro-2H-pyrano[3,4-c]isoquinolin-1-yl)-N-methyl-1H-pyrrolo[3,2-c]pyridine-2-carboxamide;
5,6-difluoro-N-methyl-N-((1S)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1S,4S)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1S,4R)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1R)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1R,4S)-4,8,9-trifluoro-6-oxo-1,4,5,6-tetrahydro-2H-pyrano[3,4-c]isoquinolin-1-yl)-1H-indole-2-carboxamide;
5,6-difluoro-N-methyl-N-((1R,4R)-4,8,9-trifluoro-6-oxo-1,4,5,6-tetrahydro-2H-pyrano[3,4-c]isoquinolin-1-yl)-1H-indole-2-carboxamide;
(S)-N-(8,9-difluoro-6-oxo-1,4,5,6-tetrahydro-2H-pyrano[3,4-c]isoquinolin-1-yl)-5-fluoro-N-methyl-6-(trifluoromethyl)nicotinamide;
(R)-N-(8,9-difluoro-6-oxo-1,4,5,6-tetrahydro-2H-pyrano[3,4-c]isoquinolin-1-yl)-5-fluoro-N-methyl-6-(trifluoromethyl)nicotinamide;
(S)-5-fluoro-N-(8-fluoro-6-oxo-1,4,5,6-tetrahydro-2H-pyrano[3,4-c]isoquinolin-1-yl)-N-methyl-6-(trifluoromethyl)nicotinamide;
(R)-5-fluoro-N-(8-fluoro-6-oxo-1,4,5,6-tetrahydro-2H-pyrano[3,4-c]isoquinolin-1-yl)-N-methyl-6-(trifluoromethyl)nicotinamide;
(S)-3-chloro-N-(8,9-difluoro-6-oxo-1,4,5,6-tetrahydro-2H-pyrano[3,4-c]isoquinolin-1-yl)-N,1-dimethyl-1H-indazole-5-carboxamide;
(R)-3-chloro-N-(8,9-difluoro-6-oxo-1,4,5,6-tetrahydro-2H-pyrano[3,4-c]isoquinolin-1-yl)-N,1-dimethyl-1H-indazole-5-carboxamide;
(S)-3-chloro-N-(8,9-difluoro-6-oxo-1,4,5,6-tetrahydro-2H-pyrano[3,4-c]isoquinolin-1-yl)-1-ethyl-N-methyl-1H-indazole-5-carboxamide;
(R)-3-chloro-N-(8,9-difluoro-6-oxo-1,4,5,6-tetrahydro-2H-pyrano[3,4-c]isoquinolin-1-yl)-1-ethyl-N-methyl-1H-indazole-5-carboxamide;
(S)-N-(8,9-difluoro-6-oxo-1,4,5,6-tetrahydro-2H-pyrano[3,4-c]isoquinolin-1-yl)-1-(difluoromethyl)-N,3-dimethyl-1H-indazole-5-carboxamide;
(R)-N-(8,9-difluoro-6-oxo-1,4,5,6-tetrahydro-2H-pyrano[3,4-c]isoquinolin-1-yl)-1-(difluoromethyl)-N,3-dimethyl-1H-indazole-5-carboxamide;
(S)-5-chloro-N-(8,9-difluoro-6-oxo-1,2,3,4,5,6-hexahydrobenzo[c][1,7]naphthyridin-1-yl)-N-methyl-6-(trifluoromethyl)nicotinamide;
(R)-5-chloro-N-(8,9-difluoro-6-oxo-1,2,3,4,5,6-hexahydrobenzo[c][1,7]naphthyridin-1-yl)-N-methyl-6-(trifluoromethyl)nicotinamide;
(S)-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
(R)-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
(S)-N-(8-fluoro-6-oxo-1,4,5,6-tetrahydro-2H-pyrano[3,4-c]isoquinolin-1-yl)-N,4-dimethyl-1H-indole-2-carboxamide;
(R)-N-(8-fluoro-6-oxo-1,4,5,6-tetrahydro-2H-pyrano[3,4-c]isoquinolin-1-yl)-N,4-dimethyl-1H-indole-2-carboxamide;
(S)-4-chloro-N-(8-fluoro-6-oxo-1,4,5,6-tetrahydro-2H-pyrano[3,4-c]isoquinolin-1-yl)-N-methyl-1H-indole-2-carboxamide;
(R)-4-chloro-N-(8-fluoro-6-oxo-1,4,5,6-tetrahydro-2H-pyrano[3,4-c]isoquinolin-1-yl)-N-methyl-1H-indole-2-carboxamide;
(S)-N-(8,9-difluoro-6-oxo-1,4,5,6-tetrahydro-2H-pyrano[3,4-c]isoquinolin-1-yl)-N-methylbenzo[d]oxazole-5-carboxamide;
(R)-N-(8,9-difluoro-6-oxo-1,4,5,6-tetrahydro-2H-pyrano[3,4-c]isoquinolin-1-yl)-N-methylbenzo[d]oxazole-5-carboxamide;
(S)-N-(8,9-difluoro-6-oxo-1,4,5,6-tetrahydro-2H-pyrano[3,4-c]isoquinolin-1-yl)-N-methylbenzo[d]thiazole-5-carboxamide;
(R)-N-(8,9-difluoro-6-oxo-1,4,5,6-tetrahydro-2H-pyrano[3,4-c]isoquinolin-1-yl)-N-methylbenzo[d]thiazole-5-carboxamide;
(S)-N-(8,9-difluoro-6-oxo-1,4,5,6-tetrahydro-2H-pyrano[3,4-c]isoquinolin-1-yl)-N-methylbenzo[d]thiazole-6-carboxamide;
(R)-N-(8,9-difluoro-6-oxo-1,4,5,6-tetrahydro-2H-pyrano[3,4-c]isoquinolin-1-yl)-N-methylbenzo[d]thiazole-6-carboxamide;
(S)-N-(8,9-difluoro-6-oxo-1,4,5,6-tetrahydro-2H-pyrano[3,4-c]isoquinolin-1-yl)-N-methyl-1H-indazole-5-carboxamide;
(R)-N-(8,9-difluoro-6-oxo-1,4,5,6-tetrahydro-2H-pyrano[3,4-c]isoquinolin-1-yl)-N-methyl-1H-indazole-5-carboxamide;
(S)-N-(8,9-difluoro-6-oxo-1,2,3,4,5,6-hexahydrobenzo[c][1,7]naphthyridin-1-yl)-8-(difluoromethyl)-N-methylindolizine-2-carboxamide;
(R)-N-(8,9-difluoro-6-oxo-1,2,3,4,5,6-hexahydrobenzo[c][1,7]naphthyridin-1-yl)-8-(difluoromethyl)-N-methylindolizine-2-carboxamide;
(S)-N-(8,9-difluoro-6-oxo-1,2,3,4,5,6-hexahydrobenzo[c][1,7]naphthyridin-1-yl)-5-(difluoromethyl)-N-methylindolizine-2-carboxamide;
(R)-N-(8,9-difluoro-6-oxo-1,2,3,4,5,6-hexahydrobenzo[c][1,7]naphthyridin-1-yl)-5-(difluoromethyl)-N-methylindolizine-2-carboxamide;
(S)-N-(8,9-difluoro-6-oxo-1,2,3,4,5,6-hexahydrobenzo[c][1,7]naphthyridin-1-yl)-5-fluoro-N-methyl-6-(trifluoromethyl)nicotinamide;
(R)-N-(8,9-difluoro-6-oxo-1,2,3,4,5,6-hexahydrobenzo[c][1,7]naphthyridin-1-yl)-5-fluoro-N-methyl-6-(trifluoromethyl)nicotinamide;
(S)-N-(8,9-difluoro-6-oxo-1,4,5,6-tetrahydro-2H-pyrano[3,4-c]isoquinolin-1-yl)-3-(difluoromethyl)-N,1-dimethyl-1H-indazole-5-carboxamide;
(R)-N-(8,9-difluoro-6-oxo-1,4,5,6-tetrahydro-2H-pyrano[3,4-c]isoquinolin-1-yl)-3-(difluoromethyl)-N,1-dimethyl-1H-indazole-5-carboxamide;
(S)-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
(R)-N-(8,9-difluoro-6-oxo-1,4,5,6-tetrahydro-2H-pyrano[3,4-c]isoquinolin-1-yl)-1-(difluoromethyl)-N-methyl-1H-indazole-5-carboxamide;
(S)-4-chloro-6-fluoro-N-(8-fluoro-6-oxo-1,4,5,6-tetrahydro-2H-pyrano[3,4-c]isoquinolin-1-yl)-N-methyl-1H-indole-2-carboxamide;
(R)-4-chloro-6-fluoro-N-(8-fluoro-6-oxo-1,4,5,6-tetrahydro-2H-pyrano[3,4-c]isoquinolin-1-yl)-N-methyl-1H-indole-2-carboxamide;
(S)-4-chloro-N-(8,9-difluoro-6-oxo-1,2,3,4,5,6-hexahydrobenzo[c][1,7]naphthyridin-1-yl)-N-methyl-1H-indole-2-carboxamide;
(R)-4-chloro-N-(8,9-difluoro-6-oxo-1,2,3,4,5,6-hexahydrobenzo[c][1,7]naphthyridin-1-yl)-N-methyl-1H-indole-2-carboxamide;
(S)-N-(8-fluoro-6-oxo-1,4,5,6-tetrahydro-2H-pyrano[3,4-c]isoquinolin-1-yl)-N-methyl-2,3-dihydro-1H-indene-5-carboxamide;
(R)-N-(8-fluoro-6-oxo-1,4,5,6-tetrahydro-2H-pyrano[3,4-c]isoquinolin-1-yl)-N-methyl-2,3-dihydro-1H-indene-5-carboxamide;
(S)-N-(8-fluoro-6-oxo-1,4,5,6-tetrahydro-2H-pyrano[3,4-c]isoquinolin-1-yl)-N-methylbenzo[d]thiazole-5-carboxamide;
(R)-N-(8-fluoro-6-oxo-1,4,5,6-tetrahydro-2H-pyrano[3,4-c]isoquinolin-1-yl)-N-methylbenzo[d]thiazole-5-carboxamide;
(S)-N-(8-fluoro-6-oxo-1,4,5,6-tetrahydro-2H-pyrano[3,4-c]isoquinolin-1-yl)-N-methylbenzo[d]thiazole-6-carboxamide;
(R)-N-(8-fluoro-6-oxo-1,4,5,6-tetrahydro-2H-pyrano[3,4-c]isoquinolin-1-yl)-N-methylbenzo[d]thiazole-6-carboxamide;
(S)-N-(8-fluoro-6-oxo-1,4,5,6-tetrahydro-2H-pyrano[3,4-c]isoquinolin-1-yl)-N-methyl-1H-indazole-5-carboxamide;
(R)-N-(8-fluoro-6-oxo-1,4,5,6-tetrahydro-2H-pyrano[3,4-c]isoquinolin-1-yl)-N-methyl-1H-indazole-5-carboxamide;
(S)-N-(8,9-difluoro-6-oxo-1,4,5,6-tetrahydro-2H-pyrano[3,4-c]isoquinolin-1-yl)-N-methyl-1H-indazole-6-carboxamide;
(R)-N-(8,9-difluoro-6-oxo-1,4,5,6-tetrahydro-2H-pyrano[3,4-c]isoquinolin-1-yl)-N-methyl-1H-indazole-6-carboxamide;
(S)-N-(8,9-difluoro-6-oxo-1,4,5,6-tetrahydro-2H-pyrano[3,4-c]isoquinolin-1-yl)-N-methyl-1H-benzo[d]imidazole-6-carboxamide;
(R)-N-(8,9-difluoro-6-oxo-1,4,5,6-tetrahydro-2H-pyrano[3,4-c]isoquinolin-1-yl)-N-methyl-1H-benzo[d]imidazole-6-carboxamide;
(R)-2-chloro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-2-chloro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-2-chloro-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
(S)-2-chloro-N-(8,9-difluoro-6-oxo-1,2,3,4,5,6-hexahydrobenzo[c][1,7]naphthyridin-1-yl)-N-methyl-4H-thieno[3,2-b]pyrrole-5-carboxamide;
(R)-6-fluoro-N-(8-fluoro-6-oxo-1,4,5,6-tetrahydro-2H-pyrano[3,4-c]isoquinolin-1-yl)-N,4-dimethyl-1H-indole-2-carboxamide;
(S)-6-fluoro-N-(8-fluoro-6-oxo-1,4,5,6-tetrahydro-2H-pyrano[3,4-c]isoquinolin-1-yl)-N,4-dimethyl-1H-indole-2-carboxamide;
(R)-N-(8,9-difluoro-6-oxo-1,2,3,4,5,6-hexahydrobenzo[c][1,7]naphthyridin-1-yl)-N,4-dimethyl-1H-indole-2-carboxamide;
(S)-N-(8,9-difluoro-6-oxo-1,2,3,4,5,6-hexahydrobenzo[c][1,7]naphthyridin-1-yl)-N,4-dimethyl-1H-indole-2-carboxamide;
(R)-N-(8,9-difluoro-6-oxo-1,2,3,4,5,6-hexahydrobenzo[c][1,7]naphthyridin-1-yl)-6-fluoro-N,4-dimethyl-1H-indole-2-carboxamide;
(S)-N-(8,9-difluoro-6-oxo-1,2,3,4,5,6-hexahydrobenzo[c][1,7]naphthyridin-1-yl)-6-fluoro-N,4-dimethyl-1H-indole-2-carboxamide;
(R)-4-chloro-N-(8,9-difluoro-6-oxo-1,2,3,4,5,6-hexahydrobenzo[c][1,7]naphthyridin-1-yl)-6-fluoro-N-methyl-1H-indole-2-carboxamide;
(S)-4-chloro-N-(8,9-difluoro-6-oxo-1,2,3,4,5,6-hexahydrobenzo[c][1,7]naphthyridin-1-yl)-6-fluoro-N-methyl-1H-indole-2-carboxamide;
(R)-N-(8,9-difluoro-6-oxo-1,2,3,4,5,6-hexahydrobenzo[c][1,7]naphthyridin-1-yl)-N-methylbenzo[d]thiazole-5-carboxamide;
(S)-N-(8,9-difluoro-6-oxo-1,2,3,4,5,6-hexahydrobenzo[c][1,7]naphthyridin-1-yl)-N-methylbenzo[d]thiazole-5-carboxamide;
(R)-N-(8,9-difluoro-6-oxo-1,2,3,4,5,6-hexahydrobenzo[c][1,7]naphthyridin-1-yl)-N-methylbenzo[d]thiazole-6-carboxamide;
(S)-N-(8,9-difluoro-6-oxo-1,2,3,4,5,6-hexahydrobenzo[c][1,7]naphthyridin-1-yl)-N-methylbenzo[d]thiazole-6-carboxamide;
(R)-N-(8,9-difluoro-6-oxo-1,2,3,4,5,6-hexahydrophenanthridin-1-yl)-N-methyl-1H-indole-2-carboxamide;
(S)-N-(8,9-difluoro-6-oxo-1,2,3,4,5,6-hexahydrophenanthridin-1-yl)-N-methyl-1H-indole-2-carboxamide;
(S)-2-cyano-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-2-cyano-N-(8,9-difluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-N-(8-fluoro-6-oxo-1,4,5,6-tetrahydro-2H-pyrano[3,4-c]isoquinolin-1-yl)-N-methyl-1H-indazole-6-carboxamide;
(S)-N-(8-fluoro-6-oxo-1,4,5,6-tetrahydro-2H-pyrano[3,4-c]isoquinolin-1-yl)-N-methyl-1H-indazole-6-carboxamide;
(R)-N-(8,9-difluoro-6-oxo-1,2,3,4,5,6-hexahydrobenzo[c][1,7]naphthyridin-1-yl)-N-methyl-2,3-dihydro-1H-indene-5-carboxamide;
(S)-N-(8,9-difluoro-6-oxo-1,2,3,4,5,6-hexahydrobenzo[c][1,7]naphthyridin-1-yl)-N-methyl-2,3-dihydro-1H-indene-5-carboxamide;
(R)-N-(8,9-difluoro-6-oxo-1,2,3,4,5,6-hexahydrobenzo[c][1,7]naphthyridin-1-yl)-N-methyl-1H-indazole-5-carboxamide;
(S)-N-(8,9-difluoro-6-oxo-1,2,3,4,5,6-hexahydrobenzo[c][1,7]naphthyridin-1-yl)-N-methyl-1H-indazole-5-carboxamide;
(R)-N-(8-fluoro-6-oxo-1,2,3,4,5,6-hexahydrophenanthridin-1-yl)-N-methyl-1H-indole-2-carboxamide;
(S)-N-(8-fluoro-6-oxo-1,2,3,4,5,6-hexahydrophenanthridin-1-yl)-N-methyl-1H-indole-2-carboxamide;
(S)-2-chloro-N-(8,9-difluoro-4,6-dioxo-1,4,5,6-tetrahydro-2H-pyrano[3,4-c]isoquinolin-1-yl)-N-methyl-4H-thieno[3,2-b]pyrrole-5-carboxamide;
(R)-2-chloro-N-(8,9-difluoro-4,6-dioxo-1,4,5,6-tetrahydro-2H-pyrano[3,4-c]isoquinolin-1-yl)-N-methyl-4H-thieno[3,2-b]pyrrole-5-carboxamide;
(S)-N-(8,9-difluoro-4,6-dioxo-1,4,5,6-tetrahydro-2H-pyrano[3,4-c]isoquinolin-1-yl)-6-(difluoromethyl)-5-fluoro-N-methyl-1H-indole-2-carboxamide;
(R)-N-(8,9-difluoro-4,6-dioxo-1,4,5,6-tetrahydro-2H-pyrano[3,4-c]isoquinolin-1-yl)-6-(difluoromethyl)-5-fluoro-N-methyl-1H-indole-2-carboxamide;
(R)-6-(difluoromethyl)-5-fluoro-N-(8-fluoro-6-oxo-1,2,3,4,5,6-hexahydrophenanthridin-1-yl)-N-methyl-1H-indole-2-carboxamide;
(S)-6-(difluoromethyl)-5-fluoro-N-(8-fluoro-6-oxo-1,2,3,4,5,6-hexahydrophenanthridin-1-yl)-N-methyl-1H-indole-2-carboxamide;
(R)-N-(8,9-difluoro-6-oxo-1,2,3,4,5,6-hexahydrophenanthridin-1-yl)-5-fluoro-N-methyl-1H-indole-2-carboxamide;
(S)-N-(8,9-difluoro-6-oxo-1,2,3,4,5,6-hexahydrophenanthridin-1-yl)-5-fluoro-N-methyl-1H-indole-2-carboxamide;
(R)-N-(8,9-difluoro-6-oxo-1,2,3,4,5,6-hexahydrophenanthridin-1-yl)-5,6-difluoro-N-methyl-1H-indole-2-carboxamide;
(S)-N-(8,9-difluoro-6-oxo-1,2,3,4,5,6-hexahydrophenanthridin-1-yl)-5,6-difluoro-N-methyl-1H-indole-2-carboxamide;
(R)-N-(8,9-difluoro-6-oxo-1,2,3,4,5,6-hexahydrophenanthridin-1-yl)-6-(difluoromethyl)-5-fluoro-N-methyl-1H-indole-2-carboxamide;
(S)-N-(8,9-difluoro-6-oxo-1,2,3,4,5,6-hexahydrophenanthridin-1-yl)-6-(difluoromethyl)-5-fluoro-N-methyl-1H-indole-2-carboxamide;
(R)-N-(8,9-difluoro-6-oxo-1,2,3,4,5,6-hexahydrophenanthridin-1-yl)-4-(difluoromethyl)-6-fluoro-N-methyl-1H-indole-2-carboxamide;
(S)-N-(8,9-difluoro-6-oxo-1,2,3,4,5,6-hexahydrophenanthridin-1-yl)-4-(difluoromethyl)-6-fluoro-N-methyl-1H-indole-2-carboxamide;
(R)-N-(8,9-difluoro-6-oxo-1,2,3,4,5,6-hexahydrophenanthridin-1-yl)-N,4-dimethyl-1H-indole-2-carboxamide;
(S)-N-(8,9-difluoro-6-oxo-1,2,3,4,5,6-hexahydrophenanthridin-1-yl)-N,4-dimethyl-1H-indole-2-carboxamide;
(R)-N-(8,9-difluoro-6-oxo-1,2,3,4,5,6-hexahydrophenanthridin-1-yl)-6-fluoro-N,4-dimethyl-1H-indole-2-carboxamide;
(S)-N-(8,9-difluoro-6-oxo-1,2,3,4,5,6-hexahydrophenanthridin-1-yl)-6-fluoro-N,4-dimethyl-1H-indole-2-carboxamide;
(S)-1-((4-bromo-3-fluorobenzyl)(methyl)amino)-8,9-difluoro-1,5-dihydro-2H-pyrano[3,4-c]isoquinolin-6(4H)-one;
(R)-1-((4-bromo-3-fluorobenzyl)(methyl)amino)-8,9-difluoro-1,5-dihydro-2H-pyrano[3,4-c]isoquinolin-6(4H)-one;
2-chloro-N-((1S)-8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
(S,R)-2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
(S,S)-2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
2-chloro-N-((1R)-8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
(R,R)-2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
(R,S)-2-chloro-N-(8,9-difluoro-4-hydroxy-6-oxo-1,4,5,6-tetrahydro-2H-pyrano[3,4-c]isoquinolin-1-yl)-N-methyl-4H-thieno[3,2-b]pyrrole-5-carboxamide;
N-((1R)-8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(R,R)-N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(R,S)-N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
N-((1S)-8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(S,S)-N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(S,R)-N-(8,9-difluoro-4-hydroxy-6-oxo-1,4,5,6-tetrahydro-2H-pyrano[3,4-c]isoquinolin-1-yl)-6-(difluoromethyl)-5-fluoro-N-methyl-1H-indole-2-carboxamide;
(R)-5-fluoro-N-(8-fluoro-6-oxo-1,2,3,4,5,6-hexahydrophenanthridin-1-yl)-N-methyl-1H-indole-2-carboxamide;
(S)-5-fluoro-N-(8-fluoro-6-oxo-1,2,3,4,5,6-hexahydrophenanthridin-1-yl)-N-methyl-1H-indole-2-carboxamide;
(R)-5,6-difluoro-N-(8-fluoro-6-oxo-1,2,3,4,5,6-hexahydrophenanthridin-1-yl)-N-methyl-1H-indole-2-carboxamide;
(S)-5,6-difluoro-N-(8-fluoro-6-oxo-1,2,3,4,5,6-hexahydrophenanthridin-1-yl)-N-methyl-1H-indole-2-carboxamide;
(R)-N-(8-fluoro-6-oxo-1,2,3,4,5,6-hexahydrophenanthridin-1-yl)-N,4-dimethyl-1H-indole-2-carboxamide;
(S)-N-(8-fluoro-6-oxo-1,2,3,4,5,6-hexahydrophenanthridin-1-yl)-N,4-dimethyl-1H-indole-2-carboxamide;
(R)-6-fluoro-N-(8-fluoro-6-oxo-1,2,3,4,5,6-hexahydrophenanthridin-1-yl)-N,4-dimethyl-1H-indole-2-carboxamide;
(S)-6-fluoro-N-(8-fluoro-6-oxo-1,2,3,4,5,6-hexahydrophenanthridin-1-yl)-N,4-dimethyl-1H-indole-2-carboxamide;
(R)-4-(difluoromethyl)-6-fluoro-N-(8-fluoro-6-oxo-1,2,3,4,5,6-hexahydrophenanthridin-1-yl)-N-methyl-1H-indole-2-carboxamide;
(S)-4-(difluoromethyl)-6-fluoro-N-(8-fluoro-6-oxo-1,2,3,4,5,6-hexahydrophenanthridin-1-yl)-N-methyl-1H-indole-2-carboxamide;
(S)-1-(((5,6-difluoro-1H-indol-2-yl)methyl)(methyl)amino)-8,9-difluoro-1,5-dihydro-2H-pyrano[3,4-c]isoquinolin-6(4H)-one;
(R)-1-(((5,6-difluoro-1H-indol-2-yl)methyl)(methyl)amino)-8,9-difluoro-1,5-dihydro-2H-pyrano[3,4-c]isoquinolin-6(4H)-one;
(S)-N-(8,9-difluoro-6-oxo-1,2,3,4,5,6-hexahydrobenzo[c][1,7]naphthyridin-1-yl)-N-methyl-1H-indazole-6-carboxamide;
(R)-N-(8,9-difluoro-6-oxo-1,2,3,4,5,6-hexahydrobenzo[c][1,7]naphthyridin-1-yl)-N-methyl-1H-indazole-6-carboxamide;
(S)-2-fluoro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(R)-2-fluoro-N-(8-fluoro-6-oxo-1,4,5,6-tetrahydro-2H-pyrano[3,4-c]isoquinolin-1-yl)-N-methyl-4H-thieno[3,2-b]pyrrole-5-carboxamide;
(S)-N-(8,9-difluoro-6-oxo-1,2,3,4,5,6-hexahydrobenzo[c][1,7]naphthyridin-1-yl)-2-fluoro-N-methyl-4H-thieno[3,2-b]pyrrole-5-carboxamide;
(R)-N-(8,9-difluoro-6-oxo-1,2,3,4,5,6-hexahydrobenzo[c][1,7]naphthyridin-1-yl)-2-fluoro-N-methyl-4H-thieno[3,2-b]pyrrole-5-carboxamide;
N-((1R)-8,9-difluoro-4-hydroxy-6-oxo-1,2,3,4,5,6-hexahydrophenanthridin-1-yl)-5-fluoro-N-methyl-1H-indole-2-carboxamide;
N-((1S)-8,9-difluoro-4-hydroxy-6-oxo-1,2,3,4,5,6-hexahydrophenanthridin-1-yl)-5-fluoro-N-methyl-1H-indole-2-carboxamide;
(R,R)-N-(8,9-difluoro-4-hydroxy-6-oxo-1,2,3,4,5,6-hexahydrophenanthridin-1-yl)-5-fluoro-N-methyl-1H-indole-2-carboxamide;
(S,R)-N-(8,9-difluoro-4-hydroxy-6-oxo-1,2,3,4,5,6-hexahydrophenanthridin-1-yl)-5-fluoro-N-methyl-1H-indole-2-carboxamide;
(S,S)-N-(8,9-difluoro-4-hydroxy-6-oxo-1,2,3,4,5,6-hexahydrophenanthridin-1-yl)-5-fluoro-N-methyl-1H-indole-2-carboxamide;
(R,S)-N-(8,9-difluoro-4-hydroxy-6-oxo-1,2,3,4,5,6-hexahydrophenanthridin-1-yl)-5-fluoro-N-methyl-1H-indole-2-carboxamide;
N-((1R)-8,9-difluoro-4-hydroxy-6-oxo-1,2,3,4,5,6-hexahydrophenanthridin-1-yl)-5,6-difluoro-N-methyl-1H-indole-2-carboxamide;
N-((1S)-8,9-difluoro-4-hydroxy-6-oxo-1,2,3,4,5,6-hexahydrophenanthridin-1-yl)-5,6-difluoro-N-methyl-1H-indole-2-carboxamide;
(S,R)-N-(8,9-difluoro-4-hydroxy-6-oxo-1,2,3,4,5,6-hexahydrophenanthridin-1-yl)-5,6-difluoro-N-methyl-1H-indole-2-carboxamide;
(R,R)-N-(8,9-difluoro-4-hydroxy-6-oxo-1,2,3,4,5,6-hexahydrophenanthridin-1-yl)-5,6-difluoro-N-methyl-1H-indole-2-carboxamide;
(R,S)-N-(8,9-difluoro-4-hydroxy-6-oxo-1,2,3,4,5,6-hexahydrophenanthridin-1-yl)-5,6-difluoro-N-methyl-1H-indole-2-carboxamide;
(S,S)-N-(8,9-difluoro-4-hydroxy-6-oxo-1,2,3,4,5,6-hexahydrophenanthridin-1-yl)-5,6-difluoro-N-methyl-1H-indole-2-carboxamide;
N-((1R)-8,9-difluoro-4-hydroxy-6-oxo-1,2,3,4,5,6-hexahydrophenanthridin-1-yl)-N-methyl-1H-indole-2-carboxamide;
N-((1S)-8,9-difluoro-4-hydroxy-6-oxo-1,2,3,4,5,6-hexahydrophenanthridin-1-yl)-N-methyl-1H-indole-2-carboxamide;
(S,R)-N-(8,9-difluoro-4-hydroxy-6-oxo-1,2,3,4,5,6-hexahydrophenanthridin-1-yl)-N-methyl-1H-indole-2-carboxamide;
(R,R)-N-(8,9-difluoro-4-hydroxy-6-oxo-1,2,3,4,5,6-hexahydrophenanthridin-1-yl)-N-methyl-1H-indole-2-carboxamide;
(R,S)-N-(8,9-difluoro-4-hydroxy-6-oxo-1,2,3,4,5,6-hexahydrophenanthridin-1-yl)-N-methyl-1H-indole-2-carboxamide;
(S,S)-N-(8,9-difluoro-4-hydroxy-6-oxo-1,2,3,4,5,6-hexahydrophenanthridin-1-yl)-N-methyl-1H-indole-2-carboxamide;
(R)-N-(8,9-difluoro-6-oxo-1,2,3,4,5,6-hexahydrobenzo[c][1,7]naphthyridin-1-yl)-N-methylindolizine-2-carboxamide;
(S)-N-(8,9-difluoro-6-oxo-1,2,3,4,5,6-hexahydrobenzo[c][1,7]naphthyridin-1-yl)-N-methylindolizine-2-carboxamide;
(R)-8-chloro-N-(8,9-difluoro-6-oxo-1,2,3,4,5,6-hexahydrobenzo[c][1,7]naphthyridin-1-yl)-N-methylindolizine-2-carboxamide;
(S)-8-chloro-N-(8,9-difluoro-6-oxo-1,2,3,4,5,6-hexahydrobenzo[c][1,7]naphthyridin-1-yl)-N-methylindolizine-2-carboxamide;
(S)-N-(8,9-Difluoro-6-oxo-1,4,5,6-tetrahydro-2H-pyrano[3,4-c]isoquinolin-1-yl)-2,2-difluoro-N-methyl-2-phenylacetamide;
(R)-N-(8,9-Difluoro-6-oxo-1,4,5,6-tetrahydro-2H-pyrano[3,4-c]isoquinolin-1-yl)-2,2-difluoro-N-methyl-2-phenylacetamide;
(S)-N-(8,9-Difluoro-6-oxo-1,4,5,6-tetrahydro-2H-pyrano[3,4-c]isoquinolin-1-yl-1-d)-5,6-difluoro-N-(methyl-13C-d3)-1H-indole-2-carboxamide;
(R)-N-(8,9-Difluoro-6-oxo-1,4,5,6-tetrahydro-2H-pyrano[3,4-c]isoquinolin-1-yl-1-d)-5,6-difluoro-N-(methyl-13C-d3)-1H-indole-2-carboxamide;
(S)-N-(8,9-Difluoro-6-oxo-1,4,5,6-tetrahydro-2H-pyrano[3,4-c]isoquinolin-1-yl-1-d)-6-(difluoromethyl)-5-fluoro-N-(methyl-13C-d3)-1H-indole-2-carboxamide;
(R)-N-(8,9-Difluoro-6-oxo-1,4,5,6-tetrahydro-2H-pyrano[3,4-c]isoquinolin-1-yl-1-d)-6-(difluoromethyl)-5-fluoro-N-(methyl-13C-d3)-1H-indole-2-carboxamide;
(S)-2-Chloro-N-(8,9-difluoro-6-oxo-1,4,5,6-tetrahydro-2H-pyrano[3,4-c]isoquinolin-1-yl-1-d)-N-(methyl-13C-d3)-4H-thieno[3,2-b]pyrrole-5-carboxamide; and
(R)-2-Chloro-N-(8,9-difluoro-6-oxo-1,4,5,6-tetrahydro-2H-pyrano[3,4-c]isoquinolin-1-yl-1-d)-N-(methyl-13C-d₃)-4H-thieno[3,2-b]pyrrole-5-carboxamide;
or a salt, solvate, prodrug, isotopically labelled derivative, stereoisomer, or tautomer thereof, or any mixtures thereof.

14. A pharmaceutical composition comprising at least one compound of claim 1 and a pharmaceutically acceptable carrier.

15. The pharmaceutical composition of claim 14, further comprising at least one additional agent useful for treating, ameliorating, or preventing hepatitis B virus infection, optionally wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

16. (canceled)

17. A method of treating, ameliorating or preventing hepatitis B virus (HBV) infection in a subject, the method comprising administering to the subject in need thereof a therapeutically effective amount of at least one compound of claim 1, optionally wherein the at least one compound is administered to the subject in a pharmaceutically acceptable composition.

18. The method of claim 17, wherein the subject is further infected with hepatitis D virus (HDV).

19. (canceled)

20. The method of claim 17, wherein the subject is further administered at least one additional agent useful for treating, ameliorating, or preventing the hepatitis B virus infection.

21. The method of claim 20, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

22. The method of claim 21, wherein the immunostimulator is a checkpoint inhibitor, optionally wherein the checkpoint inhibitor is a PD-L1 inhibitor.

23. (canceled)

24. The method of claim 20, wherein the subject is co-administered the at least one compound and the at least one additional agent, optionally wherein the at least one compound and the at least one additional agent are coformulated.

25. (canceled)

26. A method of inhibiting expression or function of a viral capsid protein directly or indirectly in a hepatitis B virus-infected subject, the method comprising administering to the subject in need thereof a therapeutically effective amount of at least one compound of claim 1, optionally wherein the at least one compound is administered to the subject in a pharmaceutically acceptable composition.

27. The method of claim 26, wherein the subject is further infected with hepatitis D virus (HDV).

28. (canceled)

29. The method of claim 26, wherein the subject is further administered at least one additional agent useful for treating, ameliorating, preventing the hepatitis B viral infection.

30. The method of claim 29, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; GalNAc-siRNA conjugate targeted against an HBV gene transcript; and therapeutic vaccine.

31. The method of claim 30, wherein the immunostimulator is a checkpoint inhibitor, optionally wherein the checkpoint inhibitor is a PD-L1 inhibitor.

32. (canceled)

33. The method of claim 29, wherein the subject is co-administered the at least one compound and the at least one additional agent, optionally wherein the at least one compound and the at least one additional agent are coformulated.

34. (canceled)

35. The method of claim 17, wherein the subject is a mammal, optionally wherein the mammal is a human.

36. The method of claim 26, wherein the subject is a mammal, optionally wherein the mammal is a human.
